(19) United States
(12) Patent Application Publication
(10) Pub. No.: US 2023/0059136 A1
(43) Pub. Date: Feb. 23, 2023

(54) THE COMPOUNDS AND COMPOSITIONS FOR TREATING CONDITIONS ASSOCIATED WITH NLRP ACTIVITY

(71) Applicant: Novartis AG, Basel (CH)

(72) Inventors: Luigi FRANCHI, Ann Arbor, MI (US); Shomir GHOSH, Brookline, MA (US); Gary GLICK, Ann Arbor, MI (US); Jason KATZ, Newton, MA (US); Anthony William OPIPARI, Jr., Dexter, MI (US); William ROUSH, Boston, MA (US); Hans Martin SEIDEL, Concord, MA (US); Dong-Ming SHEN, Edison, NJ (US); Shankar VENKATRAMAN, Lansdale, PA (US); David Guenther WINKLER, Arlington, MA (US)

(21) Appl. No.: 17/293,787

(22) PCT Filed: Nov. 14, 2019
(86) PCT No.: PCT/US2019/061531

§ 371 Date: May 13, 2021

Related U.S. Application Data
(60) Provisional application No. 62/768,805, filed on Nov. 16, 2018.
(60) Provisional application No. 62/769,165, filed on Nov. 19, 2018.
(60) Provisional application No. 62/769,151, filed on Nov. 19, 2018.
(60) Provisional application No. 62/795,981, filed on Jan. 23, 2019.
(60) Provisional application No. 62/796,356, filed on Jan. 24, 2019.
(60) Provisional application No. 62/796,361, filed on Jan. 24, 2019.
(60) Provisional application No. 62/898,241, filed on Sep. 10, 2019.

(51) Int. Cl. C07D 277/80; C07D 333/34; C07C 311/57; C07D 231/12; C07D 317/60; C07D 307/36; C07D 417/12

(52) U.S. Cl. CPC C07D 277/80; C07D 333/34; C07C 311/57; C07D 417/12; C07D 317/60; C07D 307/36; C07D 231/12

(57) ABSTRACT

In one aspect, compounds of Formula AA, or a pharmaceutically acceptable salt thereof, are featured: Formula AA or a pharmaceutically acceptable salt thereof, wherein the variables shown in Formula A can be as defined anywhere herein.

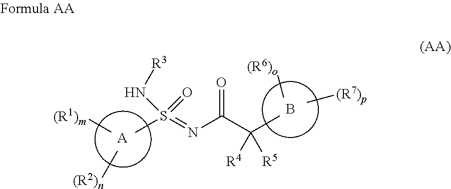

### TECHNICAL FIELD

This disclosure features chemical entities (e.g., a compound that modulates (e.g., antagonizes) NLRP3, or a pharmaceutically acceptable salt, and/or hydrate, and/or cocrystal, and/or drug combination of the compound) that are useful, e.g., for treating a condition, disease or disorder in which a decrease or increase in NLRP3 activity (e.g., an increase, e.g., a condition, disease or disorder associated with NLRP3 signaling) contributes to the pathology and/or symptoms and/or progression of the condition, disease or disorder in a subject (e.g., a human). This disclosure also features compositions as well as other methods of using and making the same.

The present disclosure also relates to, in part, methods and compositions for treating anti-TNFα resistance in a subject with an NLRP3 antagonist. The present disclosure also relates, in part, to methods, combinations and compositions for treating TNFα related diseases and anti-TNFα resistance in a subject that include administration of an NLRP3 antagonist, an NLRP3 antagonist and an anti-TNFα agent, or a composition encompassing an NLRP3 antagonist and an anti-TNFα agent.

### BACKGROUND

The NLRP3 inflammasome is a component of the inflammatory process and its aberrant activation is pathogenic in inherited disorders such as the cryopyrin associated periodic syndromes (CAPS). The inherited CAPS Muckle-Wells syndrome (MWS), familial cold autoinflammatory syndrome (FCAS) and neonatal onset multi-system inflammatory disease (NOMID) are examples of indications that have been reported to be associated with gain of function mutations in NLRP3.

NLRP3 can form a complex and has been implicated in the pathogenesis of a number of complex diseases, including but not limited to metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as Osteoarthritis, osteoporosis and osteopetrosis disorders eye disease, such as glaucoma and macular degeneration, diseased caused by viral infection such as HIV and AIDS, autoimmune disease such as Rheumatoid Arthritis, Systemic Lupus Erythematosus, Autoimmune Thyroiditis, Addison's disease, pernicious anemia, cancer and aging.

In light of the above, it would be desirable to provide compounds that modulate (e.g., antagonize) NLRP3.

Several patients having inflammatory or autoimmune diseases are treated with anti-TNFα agents. A subpopulation of such patients develop resistance to treatment with the anti-TNFα agents. It is desirable to develop methods for reducing a patient's resistance to anti-TNFα agents. In light of the this, it would also be desirable to provide alternative therapies for treating inflammatory or autoimmune diseases (for example NLRP3 inflammasome inhibitors) to avoid or minimise the use of anti-TNFα agents.

Intestinal bowel disease (IBD), encompassing Ulcerative Colitis (UC) and Crohn's disease (CD), are chronic diseases characterized by barrier dysfunction and uncontrolled inflammation and mucosal immune reactions in the gut. A number of inflammatory pathways have been implicated in the progression of IBD, and anti-inflammatory therapy such as tumor necrosis factor-alpha (TNF-α) blockade has shown efficacy in the clinic (Rutgeerts P et al N Engl J Med 2005; 353:2462-76). Anti-TNFα therapies, however, do not show complete efficacy, however, other cytokines such as IL-1β, IL-6, IL-12, IL-18, IL-21, and IL-23 have been shown to drive inflammatory disease pathology in IBD (Neurath M F Nat Rev Immunol 2014; 14; 329-42). IL-1β and IL-18 are produced by the NLRP3 inflammasome in response to pathogenic danger signals, and have been shown to play a role in IBD. Anti-IL-1β therapy is efficacious in patients with IBD driven by genetic mutations in CARD8 or IL-10R (Mao L et al, J Clin Invest 2018; 238:1793-1806, Shouval D S et al, Gastroenterology 2016; 151:1100-1104), IL-18 genetic polymorphisms have been linked to UC (Kanai T et al, Curr Drug Targets 2013; 14:1392-9), and NLRP3 inflammasome inhibitors have been shown to be efficacious in murine models of IBD (Perera A P et al, Sci Rep 2018; 8:8618). Resident gut immune cells isolated from the lamina propria of IBD patients can produce IL-1β, either spontaneously or when stimulated by LPS, and this IL-1β production can be blocked by the ex vivo addition of a NLRP3 antagonist. Based on strong clinical and preclinical evidence showing that inflammasome-driven IL-1β and IL-18 play a role in IBD pathology, it is clear that NLRP3 inflammasome inhibitors could be an efficacious treatment option for UC, Crohn's disease, or subsets of IBD patients. These subsets of patients could be defined by their peripheral or gut levels of inflammasome related cytokines including IL-1β, IL-6, and IL-18, by genetic factors that pre-dispose IBD patients to having NLRP3 inflammasome activation such as mutations in genes including ATG16L1, CARD8, IL-10R, or PTPN2 (Saitoh T et al, Nature 2008; 456:264, Spalinger M R, Cell Rep 2018; 22:1835), or by other clinical rationale such as non-response to TNF therapy.

Though anti-TNF therapy is an effective treatment option for Crohn's disease, 40% of patients fail to respond. One-third of non-responsive CD patients fail to respond to anti-TNF therapy at the onset of treatment, while another third lose response to treatment over time (secondary non-response). Secondary non-response can be due to the generation of anti-drug antibodies, or a change in the immune compartment that desensitizes the patient to anti-TNF (Ben-Horin S et al, Autoimmun Rev 2014; 13:24-30, Steenholdt C et al Gut 2014; 63:919-27). Anti-TNF reduces inflammation in IBD by causing pathogenic T cell apoptosis in the intestine, therefore eliminating the T cell mediated inflammatory response (Van den Brande et al Gut 2007:56:509-17). There is increased NLRP3 expression and increased production of IL-1β in the gut of TNF-non-responsive CD patients (Leal R F et al Gut 2015; 64:233-42) compared to TNF-responsive patients, suggesting NLRP3 inflammasome pathway activation. Furthermore, there is increased expression of TNF-receptor 2 (TNF-R2), which allows for TNF-mediated proliferation of T cells (Schmitt H et al Gut 2018; 0:1-15). IL-1β signaling in the gut promotes T cell differentiation toward Th1/17 cells which can escape anti-TNF-α mediated apoptosis. It is therefore likely that NLRP3 inflammasome activation can cause non-responsiveness in CD patients to anti-TNF-α therapy by sensitizing pathogenic T cells in the gut to anti-TNF-α mediated apoptosis. Experimental data from immune cells isolated from the gut of TNF-resistant Crohn's patients show that these cells spontaneously release IL-1β, which can be inhibited by the addition of an NLRP3 antagonist. NLRP3 inflammasome antagonists—in part by blocking IL-1β secretion—would be expected to inhibit the mechanism leading to anti-TNF non-responsiveness, re-sensitizing the patient to anti-TNF therapy. In IBD patients who are naive to anti-TNF therapy, treatment with an NLRP3 antagonist would be expected to prevent primary- and secondary-non responsiveness by blocking the mechanism leading to non-response.

NLRP3 antagonists that are efficacious locally in the gut can be efficacious drugs to treat IBD; in particular in the treatment of TNF-resistant CD alone or in combination with anti-TNF therapy. Systemic inhibition of both IL-1β and TNF-α has been shown to increase the risk of opportunistic infections (Genovese M C et al, Arthritis Rheum 2004; 50:1412), therefore, only blocking the NLRP3 inflammasome at the site of inflammation would reduce the infection risk inherent in neutralizing both IL-1β and TNF-α. NLRP3 antagonists that are potent in NLRP3-inflammasome driven cytokine secretion assays in cells, but have low permeability in vitro in a permeability assay such as an MDCK assay, have poor systemic bioavailability in a rat or mouse pharmacokinetic experiment, but high levels of compound in the colon and/or small intestine could be a useful therapeutic option for gut restricted purposes.

In light of the above, the present invention also provides alternative therapies for the treatment of inflammatory or autoimmune diseases, including IBD, that solves the above problems associated with anti-TNFα agents.

### SUMMARY

This disclosure features chemical entities (e.g., a compound that modulates (e.g., antagonizes) NLRP3, or a pharmaceutically acceptable salt, and/or hydrate, and/or cocrystal, and/or drug combination of the compound) that are useful, e.g., for treating a condition, disease or disorder in which a decrease or increase in NLRP3 activity (e.g., an increase, e.g., a condition, disease or disorder associated with NLRP3 signaling) is implicated.

In some embodiments, provided herein is a compound of Formula AA

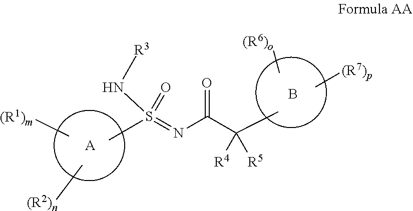

or a pharmaceutically acceptable salt thereof, wherein the variables in Formula AA can be as defined anywhere herein.

This disclosure also features compositions as well as other methods of using and making the same.

The present invention is also relates to the Applicant's discovery that inhibition of NLRP3 inflammasomes can increase a subject's sensitivity to an anti-TNFα agent or can overcome resistance to an anti-TNFα agent in a subject, or indeed provide an alternative therapy to anti-TNFα agents.

Provided herein are methods of treating a subject that include: (a) identifying a subject having a cell that has an elevated level of NLRP3 inflammasome activity and/or expression as compared to a reference level; and (b) administering to the identified subject a therapeutically effective amount of an compound of Formula I or a pharmaceutically acceptable salt, solvate, or co-crystal thereof.

Provided herein are methods for the treatment of inflammatory or autoimmune disease including IBD, such as UC and CD in a subject in need thereof, comprising administering to said subject a therapeutically effective amount a compound for Formula I or a pharmaceutically acceptable salt, solvate, or co-crystal thereof, wherein the NLRP3 antagonist is a gut-targeted NLRP3 antagonist.

Provided herein are methods of treating a subject in need thereof, that include: (a) identifying a subject having resistance to an anti-TNFα agent; and (b) administering a treatment comprising a therapeutically effective amount of a compound for Formula I, or a pharmaceutically acceptable salt, solvate, or co-crystal thereof to the identified subject.

Provided herein are methods of treating a subject in need thereof, that include: administering a treatment comprising a therapeutically effective amount of a compound for Formula I or a pharmaceutically acceptable salt, solvate, or co-crystal thereof to a subject identified as having resistance to an anti-TNFα agent.

Provided herein are methods of selecting a treatment for a subject in need thereof, that include: (a) identifying a subject having resistance to an anti-TNFα agent; and (b) selecting for the identified subject a treatment comprising a therapeutically effective amount of a compound for Formula I or a pharmaceutically acceptable salt, solvate, or co-crystal thereof.

Provided herein are methods of selecting a treatment for a subject in need thereof, that include selecting a treatment comprising a therapeutically effective amount of a compound for Formula I or a pharmaceutically acceptable salt, solvate, or co-crystal thereof for a subject identified as having resistance to an anti-TNFα agent.

In some embodiments of any of the methods described herein, the treatment further includes a therapeutically effective amount of an anti-TNFα agent, in addition to the NLRP3 antagonist.

An “antagonist” of NLRP3 includes compounds that inhibit the ability of NLRP3 to induce the production of IL-1β and/or IL-18 by directly binding to NLRP3, or by inactivating, destabilizing, altering distribution, of NLRP3 or otherwise.

In one aspect, pharmaceutical compositions are featured that include a chemical entity described herein (e.g., a compound described generically or specifically herein or a pharmaceutically acceptable salt thereof or compositions containing the same) and one or more pharmaceutically acceptable excipients.

In one aspect, methods for modulating NLRP3 activity are featured that include contacting NLRP3 with a chemical entity described herein (e.g., a compound described generically or specifically herein or a pharmaceutically acceptable salt thereof or compositions containing the same). Methods include in vitro methods, e.g., contacting a sample that includes one or more cells comprising NLRP3, as well as in vivo methods.

In a further aspect, methods of treatment of a disease in which NLRP3 signaling contributes to the pathology and/or symptoms and/or progression of the disease are featured that include administering to a subject in need of such treatment an effective amount of a chemical entity described herein (e.g., a compound described generically or specifically herein or a pharmaceutically acceptable salt thereof or compositions containing the same).

In a further aspect, methods of treatment are featured that include administering to a subject a chemical entity described herein (e.g., a compound described generically or specifically herein or a pharmaceutically acceptable salt thereof or compositions containing the same), wherein the chemical entity is administered in an amount effective to treat a disease in which NLRP3 signaling contributes to the pathology and/or symptoms and/or progression of the disease, thereby treating the disease.

Embodiments can include one or more of the following features.

The chemical entity can be administered in combination with one or more additional therapies with one or more agents suitable for the treatment of the condition, disease or disorder.

Examples of the indications that may be treated by the compounds disclosed herein include but are not limited to metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as osteoarthritis, osteoporosis and osteopetrosis disorders, eye disease, such as glaucoma and macular degeneration, diseases caused by viral infection such as HIV and AIDS, autoimmune disease such as rheumatoid arthritis, systemic Lupus erythematosus, autoimmune thyroiditis; Addison's disease, pernicious anemia, cancer and aging.

The methods can further include identifying the subject.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

As used herein, the term “NLRP3” is meant to include, without limitation, nucleic acids, polynucleotides, oligonucleotides, sense and antisense polynucleotide strands, complementary sequences, peptides, polypeptides, proteins, homologous and/or orthologous NLRP molecules, isoforms, precursors, mutants, variants, derivatives, splice variants, alleles, different species, and active fragments thereof.

The term “acceptable” with respect to a formulation, composition or ingredient, as used herein, means having no persistent detrimental effect on the general health of the subject being treated.

“API” refers to an active pharmaceutical ingredient.

The terms “effective amount” or “therapeutically effective amount,” as used herein, refer to a sufficient amount of a chemical entity (e.g., a compound exhibiting activity as a modulator of NLRP3, or a pharmaceutically acceptable salt and/or hydrate and/or cocrystal thereof) being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “pharmaceutically acceptable salt” may refer to pharmaceutically acceptable addition salts prepared from pharmaceutically acceptable non-toxic acids including inorganic and organic acids. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. The term “pharmaceutically acceptable salt” may also refer to pharmaceutically acceptable addition salts prepared by reacting a compound having an acidic group with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described herein form with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat”, “treating”, and “treatment”, in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof.

The term “prevent”, “preventing” or “prevention” in connection to a disease or disorder refers to the prophylactic treatment of a subject who is at risk of developing a condition (e.g., specific disease or disorder or clinical symptom thereof) resulting in a decrease in the probability that the subject will develop the condition.

The terms “hydrogen” and “H” are used interchangeably herein.

The term “halo” refers to fluoro (F), chloro (Cl), bromo (Br), or iodo (I).

The term “alkyl” refers to a hydrocarbon chain that may be a straight chain or branched chain, saturated or unsaturated, containing the indicated number of carbon atoms. For example, C₁₋₁₀ indicates that the group may have from 1 to 10 (inclusive) carbon atoms in it. Non-limiting examples include methyl, ethyl, iso-propyl, tert-butyl, n-hexyl.

The term “haloalkyl” refers to an alkyl, in which one or more hydrogen atoms is/are replaced with an independently selected halo.

The term “alkoxy” refers to an —O-alkyl radical (e.g., —OCH₃).

The term “carbocyclic ring” as used herein includes an aromatic or nonaromatic cyclic hydrocarbon group having 3 to 10 carbons, such as 3 to 8 carbons, such as 3 to 7 carbons, which may be optionally substituted. Examples of carbocyclic rings include five-membered, six-membered, and seven-membered carbocyclic rings.

The term “heterocyclic ring” refers to an aromatic or nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, or 3 atoms of each ring may be substituted by a substituent. Each ring of a bicyclic or tricyclic heterocyclic ring is selected from saturated, unsaturated, and aromatic (carbocyclic aromatic and heteroaromatic) rings. Examples of heterocyclic rings include five-membered, six-membered, and seven-membered heterocyclic rings.

The term “cycloalkyl” as used herein includes a nonaromatic cyclic, bicyclic, fused, or spiro hydrocarbon radical having 3 to 10 carbons, such as 3 to 8 carbons, such as 3 to 7 carbons, wherein the cycloalkyl group which may be optionally substituted. Examples of cycloalkyls include five-membered, six-membered, and seven-membered rings. Examples include cyclopropyl, cyclobutyl, cyclopentyl, cyclopentenyl, cyclohexyl, cyclohexenyl, cycloheptyl, and cyclooctyl.

The term “heterocycloalkyl” refers to a 5-8 membered monocyclic, 8-12 membered bicyclic, or 11-14 membered tricyclic fused or spiro ring system radical wherein at least one of the rings in the ring system (1) is nonaromatic and (2) includes 1-3 heteroatoms. When the ring system is bicyclic, 1-6 heteroatom ring members are present; and when the ring system is tricyclic, 1-9 heteroatom ring members are present. The ring heteroatoms are selected from O, N, and S (e.g., the ring system includes carbon atoms and 1-3, 1-6, or 1-9 heteroatoms selected from N, O, and S if monocyclic, bicyclic, or tricyclic, respectively), wherein 0, 1, 2, or 3 atoms of each ring may be substituted by a substituent. Examples of heterocycloalkyls include five-membered, six-membered, seven-membered, eight-membered, and ten-membered rings. Examples include piperazinyl, pyrrolidinyl, dioxanyl, morpholinyl, tetrahydrofuranyl, (3aR,6aS)-tetrahydro-1H-thieno[3,4-d]imidazol-2(3H)-one, isoquinoline-1,3(2H,4H)-dione, and the like.

The term “aryl” is intended to mean an aromatic ring radical containing 6 to 10 ring carbons. Examples include phenyl and naphthyl.

The term “heteroaryl” is intended to mean an aromatic ring system containing 5 to 14 aromatic ring atoms that may be a single ring, two fused rings or three fused rings wherein at least one aromatic ring atom is a heteroatom selected from, but not limited to, the group consisting of O, S and N. Examples include furanyl, thienyl, pyrrolyl, imidazolyl, oxazolyl, thiazolyl, isoxazolyl, pyrazolyl, isothiazolyl, oxadiazolyl, triazolyl, thiadiazolyl, pyridinyl, pyrazinyl, pyrimidinyl, pyridazinyl, triazinyl and the like. Examples also include carbazolyl, quinolizinyl, quinolinyl, isoquinolinyl, cinnolinyl, phthalazinyl, quinazolinyl, quinoxalinyl, triazinyl, indolyl, isoindolyl, indazolyl, indolizinyl, purinyl, naphthyridinyl, pteridinyl, carbazolyl, acridinyl. phenazinyl, phenothiazinyl, phenoxazinyl, benzoxazolyl, benzothiazolyl, 1H-benzimidazolyl, imidazopyridinyl, benzothienyl, benzofuranyl, isobenzofuran and the like.

The term “hydroxy” refers to an OH group.

The term “amino” refers to an NH₂ group.

The term “oxo” refers to O. By way of example, substitution of a CH₂ a group with oxo gives a C═O group.

As used herein, the terms “the ring A” or “A” are used interchangeably to denote

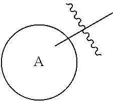

in formula AA, wherein the bond that is shown as being broken by the wavy line [symbol] connects A to the S(O)(NHR³)═N moiety of Formula AA.

As used herein, the terms “the ring B” or “B” are used interchangeably to denote

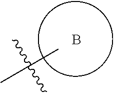

in formula AA wherein the bond that is shown as being broken by the wavy line [symbol] connects B to the C(R⁴R⁵) group of Formula AA.

As used herein, the term “the substituted ring A” is used to denote

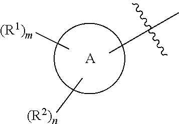

in formula AA, wherein the bond that is shown as being broken by the wavy line [symbol] connects A to the S(O)(NHR³)═N moiety of Formula AA.

As used herein, the term “the substituted ring B” is used to denote

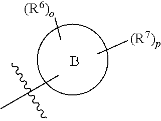

in formula AA, wherein the bond that is shown as being broken by the wavy line [symbol] connects B to the C(R⁴R⁵) group of Formula AA.

As used herein, the recitation “S(O₂)”, alone or as part of a larger recitation, refers to the group

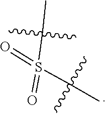 [SMILES: *S(*)(=O)=O]

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

The scope of the compounds disclosed herein includes tautomeric form of the compounds. Thus, by way of example, a compound that is represented as containing the moiety

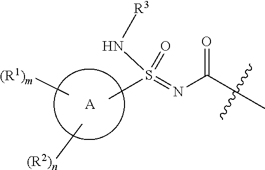

is also intended to include the tautomeric form containing the moiety

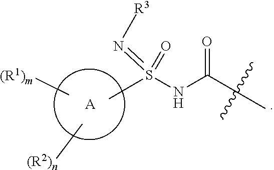

In addition, by way of example, a compound that is represented as containing the moiety

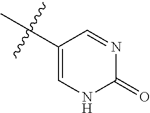 [SMILES: *c1cnc(=O)[nH]c1]

is also intended to include the tautomeric form containing the moiety

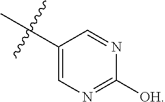 [SMILES: *c1cnc(O)nc1]

Non-limiting exemplified compounds of the formulae described herein include a stereogenic sulfur atom and optionally one or more stereogenic carbon atoms. This disclosure provides examples of stereoisomer mixtures (e.g., racemic mixture of enantiomers; mixture of diastereomers). This disclosure also describes and exemplifies methods for separating individual components of said stereoisomer mixtures (e.g., resolving the enantiomers of a racemic mixture). In cases of compounds containing only a stereogenic sulfur atom, resolved enantiomers are graphically depicted using one of the two following formats: formulas A/B (hashed and solid wedge three-dimensional representation); and formula C (“flat structures with *-labelled stereogenic sulfur).

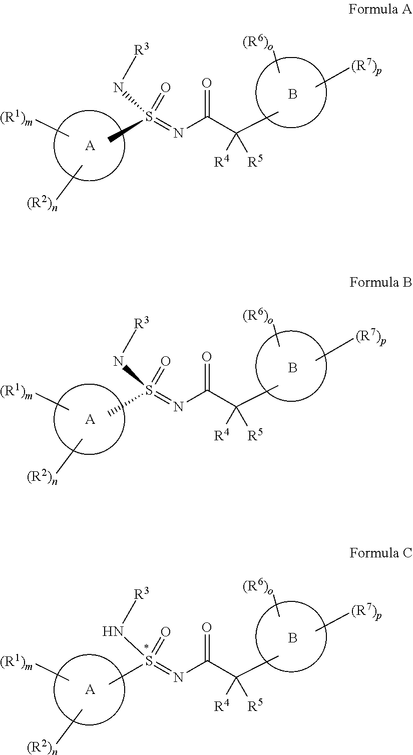

In reaction schemes showing resolution of a racemic mixture, Formulas A/B and C are intended only to convey that the constituent enantiomers were resolved in enantiopure pure form (about 98% ee or greater). The schemes that show resolution products using the formula A/B format are not intended to disclose or imply any correlation between absolute configuration and order of elution. Some of the compounds shown in the tables below are graphically represented using the formula A/B format. However, with the exception of compounds 132a and 132b, the depicted stereochemistry shown for each of the tabulated compounds drawn in the formula A/B format is a tentative assignment and based, by analogy, on the absolute stereochemistry assigned to compounds 132b (see, e.g., FIG. 1).

The details of one or more embodiments of the invention are set forth in the accompanying drawings and the description below. Other features and advantages of the invention will be apparent from the description and drawings, and from the claims.

### DESCRIPTION OF DRAWINGS

FIG. 1: Expression levels of RNA encoding NLRP3 in Crohn's Disease patients who are responsive and non-responsive to infliximab.

FIG. 2: Expression levels of RNA encoding IL-1β in Crohn's Disease patients who are responsive and non-responsive to infliximab.

FIG. 3: Expression levels of RNA encoding NLRP3 in Ulcerative Colitis (UC) patients who are responsive and non-responsive to infliximab.

FIG. 4: Expression levels of RNA encoding IL-1β in Ulcerative Colitis (UC) patients who are responsive and non-responsive to infliximab.

FIG. 5: depicts ball-and-stick representations of two crystallographically independent molecules of compound 132b in the asymmetrical unit.

FIG. 6: Layout of the microplate to measure activity of compounds in the THP-1 stimulation assay.

### DETAILED DESCRIPTION

In one aspect, provided herein is a compound of Formula AA:

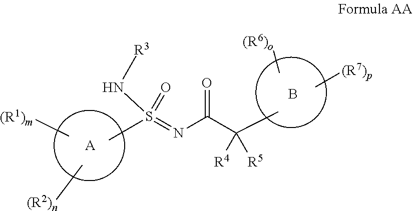

wherein

m=0, 1, or 2;

n=0, 1, or 2;

o=1 or 2;

p=0, 1, 2, or 3;

wherein

A is a 5-10-membered monocyclic or bicyclic heteroaryl or a C₆-C₁₀ monocyclic or bicyclic aryl;

B is a 5-10-membered monocyclic or bicyclic heteroaryl or a C₆-C₁₀ monocyclic or bicyclic aryl;

wherein

at least one R⁶ is ortho to the bond connecting the B ring to the C(R⁴R⁵) group of Formula AA;

R¹ and R² are each independently selected from C₁-C₆ alkyl, C₁-C₆ haloalkyl, C₁-C₆ alkoxy, C₁-C₆ haloalkoxy, halo, CN, NO₂, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NR⁸R⁹, C(O)R¹³, CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,

wherein the C₁-C₆ alkyl, C₁-C₆ alkoxy, C₁-C₆ haloalkyl, C₆-C₁₀ aryl, 5- to 10-membered heteroaryl, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, R¹⁵, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);

wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, —O(C₀-C₃ alkylene)C₆-C₁₀ aryl, halo, NR⁸R⁹, or oxo;

wherein the 3- to 7-membered heterocycloalkyl, C₆-C₁₀ aryl, and 5- to 10-membered heteroaryl are optionally substituted with one or more substituents independently selected from halo, C₁-C₆ alkyl, and OC₁-C₆ alkyl;

or one pair of R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring containing 1-3 heteroatoms independently selected from O, N, and S, wherein the carbocyclic ring or heterocyclic ring is optionally independently substituted with one or more substituents independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹,

wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, C₂-C₆ alkenyl, and C₂-C₆ alkynyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, C₃-C₁₀ cycloalkyl, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), C₆-C₁₀ aryloxy, C₃-C₁₀ cycloalkoxy, and S(O₂)C₁-C₆ alkyl; and

wherein the C₁-C₆ alkyl or C₁-C₆ alkoxy that R⁶ or R⁷ is substituted with is optionally substituted with one or more hydroxyl, C₆-C₁₀ aryl, or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;

wherein the 3- to 7-membered heterocycloalkyl, C₆-C₁₀ aryl, and 5- to 10-membered heteroaryl are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

each of R⁴ and R⁵ is independently selected from hydrogen and C₁-C₆ alkyl;

R¹⁰ is C₁-C₆ alkyl;

each of R⁸ and R⁹ at each occurrence is independently selected from hydrogen, C₁-C₆ alkyl, C₂-C₆ alkenyl, C₃-C₇ cycloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl, 3- to 7-membered heterocycloalkyl, or NR¹¹R¹²;

or R⁸ and R⁹ taken together with the nitrogen they are attached to form a 3- to 10-membered monocyclic or bicyclic ring optionally containing one or more heteroatoms in addition to the nitrogen they are attached to, wherein the ring is optionally substituted with one or more substituents independently selected from halo, C₁-C₆ alkyl, C₁-C₆ haloalkyl, C₁-C₆ alkoxy, oxo, N(C₁-C₆ alkyl)₂, NH₂, NH(C₁-C₆ alkyl), and hydroxy;

R¹³ is C₁-C₆ alkyl or —(Z¹-Z²)ₐ₁—Z³;

each of R¹¹ and R¹² at each occurrence is independently selected from hydrogen, C₁-C₆ alkyl, and —(Z¹-Z²)ₐ₁—Z³;

a1 is an integer selected from 0-10 (e.g., 0-5);

each Z¹ is independently C₁-C₆ alkylene optionally substituted with one or more substituents independently selected from oxo, halo, and hydroxy;

each Z² is independently a bond, NH, N(C₁-C₆ alkyl), —O—, —S—, or 5-10 membered heteroarylene;

Z³ is independently C₆-C₁₀ aryl, C₂-C₆ alkyenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆ alkyl)₂, NH₂, NH(C₁-C₆ alkyl), and hydroxy;

R³ is selected from hydrogen, cyano, hydroxy, C₁-C₆ alkoxy, C₁-C₆ alkyl, and

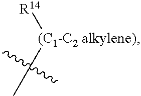

wherein the C₁-C₂ alkylene group is optionally substituted by oxo;

R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally substituted with from 1-3 independently selected R⁶;

R¹⁵ is —(Z⁴-Z⁵)ₐ₂—Z⁶;

a2 is an integer selected from 1-10 (e.g., 1-5 (e.g., 2-5));

each Z⁴ is independently selected from —O—, —S—, —NH—, and —N(C₁-C₃ alkyl)-;

provided that the Z⁴ group directly attached to R¹ or R² is —O— or —S—;

each Z⁵ is independently C₁-C₆ alkylene optionally substituted with one or more substituents independently selected from oxo, halo, and hydroxy; and

Z⁶ is OH, OC₁-C₆ alkyl, NH₂, NH(C₁-C₆ alkyl), N(C₁-C₆ alkyl)₂, NHC(O)(C₁-C₆ alkyl), NHC(O)(C₁-C₆ alkoxy), or an optionally substituted group selected from the group consisting of:

C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆ alkyl)₂, NH₂, NH(C₁-C₆ alkyl), and hydroxy;

or a pharmaceutically acceptable salt thereof.

In another aspect, provided herein is a compound of Formula AA

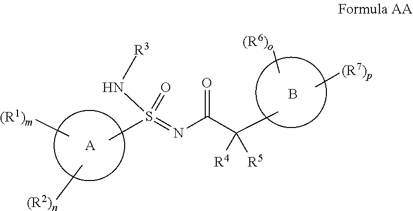

wherein

m=0, 1, or 2;

n=0, 1, or 2;

o=1 or 2;

p=0, 1, 2, or 3;

wherein

A is a 5-10-membered monocyclic or bicyclic heteroaryl or a C₆-C₁₀ monocyclic or bicyclic aryl;

B is a 5-10-membered monocyclic or bicyclic heteroaryl or a C₆-C₁₀ monocyclic or bicyclic aryl;

wherein

at least one R⁶ is ortho to the bond connecting the B ring to the C(R⁴R⁵) group of Formula AA;

R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO—C₆-C₁₀ aryl, CO-5- to 10-membered heteroaryl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,

wherein the C₁-C₆ alkyl, C₁-C₆ alkoxy, C₁-C₆ haloalkyl, C₆-C₁₀ aryl, 5- to 10-membered heteroaryl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, R¹⁵, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;

wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, —O(C₀-C₃ alkylene)C₆-C₁₀ aryl, halo, NR⁸R⁹, or oxo;

wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or one pair of R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring containing 1-3 heteroatoms independently selected from O, N, and S, wherein the carbocyclic ring or heterocyclic ring is optionally independently substituted with one or more substituents independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, C₂-C₆ alkenyl, and C₂-C₆ alkynyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, C₃-C₁₀ cycloalkyl, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, C₃-C₁₀ cycloalkoxy, and S(O₂)C₁-C₆ alkyl; and

wherein the C₁-C₆ alkyl or C₁-C₆ alkoxy that R⁶ or R⁷ is substituted with is optionally substituted with one or more hydroxyl, C₆-C₁₀ aryl, or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;

wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

each of R⁴ and R⁵ is independently selected from hydrogen and C₁-C₆ alkyl;

R¹⁰ is C₁-C₆ alkyl;

each of R⁸ and R⁹ at each occurrence is independently selected from hydrogen, C₁-C₆ alkyl, C₂-C₆ alkenyl, C₃-C₇ cycloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;

R¹³ is C₁-C₆ alkyl, C₆-C₁₀ aryl, or 5- to 10-membered heteroaryl;

each of R¹¹ and R¹² at each occurrence is independently selected from hydrogen and C₁-C₆ alkyl;

R³ is selected from hydrogen, cyano, hydroxy, C₁-C₆ alkoxy, C₁-C₆ alkyl, and

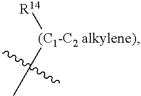

wherein the C₁-C₂ alkylene group is optionally substituted by oxo;

R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally independently substituted with from 1-3 R⁶,

R¹⁵ is —(Z⁴-Z⁵)ₐ₂—Z⁶;

a2 is an integer selected from 1-10 (e.g., 1-5 (e.g., 2-5));

each Z⁴ is independently selected from —O—, —S—, —NH—, and —N(C₁-C₃ alkyl)-;

provided that the Z⁴ group directly attached to R¹ or R² is —O— or —S—;

each Z⁵ is independently C₁-C₆ alkylene optionally substituted with one or more substituents independently selected from oxo, halo, and hydroxy; and

Z⁶ is OH, OC₁-C₆ alkyl, NH₂, NH(C₁-C₆ alkyl), N(C₁-C₆ alkyl)₂, NHC(O)(C₁-C₆ alkyl), NHC(O)(C₁-C₆ alkoxy), or an optionally substituted group selected from the group consisting of:

C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆ alkyl)₂, NH₂, NH(C₁-C₆ alkyl), and hydroxy;

or a pharmaceutically acceptable salt thereof.

In another aspect, provided herein is a compound of Formula AA

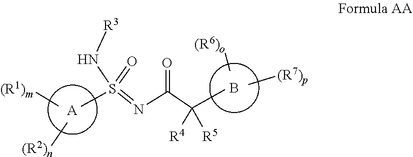

wherein
m=0, 1, or 2
n=0, 1, or 2
o=1 or 2
p=0, 1, 2, or 3
wherein
A is a 5-10-membered monocyclic or bicyclic heteroaryl or a C₆-C₁₀ monocyclic or bicyclic aryl;
B is a 5-10-membered monocyclic or bicyclic heteroaryl or a C₆-C₁₀ monocyclic or bicyclic aryl;
wherein
at least one R⁶ is ortho to the bond connecting the B ring to the C(R⁴R⁵) group of Formula AA;
R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO—C₆-C₁₀ aryl, CO-5- to 10-membered heteroaryl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₁₀ cycloalkyl and 3- to 10-membered heterocycloalkyl, and a C₂-C₆ alkenyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹′ wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
   R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
   wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen and optionally substituted with one or more halo, OH, oxo, or C₁-C₆ alkyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 4- to 8-membered heterocyclic ring containing 1 or 2 heteroatoms independently selected from O, N, NR²⁰, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
   each of R⁴ and R⁵ is independently selected from hydrogen and C₁-C₆ alkyl;
   R¹⁰ is C₁-C₆ alkyl;
   each of R⁸ and R⁹ at each occurrence is independently selected from hydrogen, C₁-C₆ alkyl, NH—(C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;
   R¹³ is C₁-C₆ alkyl, C₆-C₁₀ aryl, or 5- to 10-membered heteroaryl;
   each of R¹¹ and R¹² at each occurrence is independently selected from hydrogen and C₁-C₆ alkyl;
   R³ is selected from hydrogen, cyano, hydroxy, C₁-C₆ alkoxy, C₁-C₆ alkyl, and

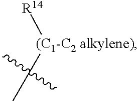

wherein the C₁-C₂ alkylene group is optionally substituted by oxo;
R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally independently substituted with 1, 2, or 3 R⁶
or a pharmaceutically acceptable salt thereof.

In another aspect, provided herein is a compound of Formula AA:

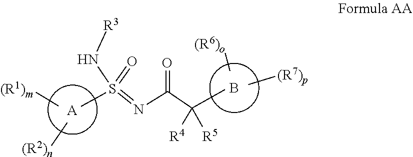

wherein

m=1 or 2;

n=1 or 2;

o=1 or 2;

p=0, 1, 2, or 3;

wherein

A is a 5-10-membered monocyclic or bicyclic heteroaryl or a C₆-C₁₀ monocyclic or bicyclic aryl;

B is a 5-10-membered monocyclic or bicyclic heteroaryl or a C₆-C₁₀ monocyclic or bicyclic aryl;

wherein

at least one R⁶ is ortho to the bond connecting the B ring to the C(R⁴R⁵) group of Formula AA;

one pair of R¹ and R² are on adjacent atoms, and taken together with the atoms connecting them, independently form one monocyclic or bicyclic C₄-C₁₂ carbocyclic ring or one monocyclic or bicyclic 5-to-12-membered heterocyclic ring containing 1-3 heteroatoms independently selected from O, N, and S, wherein the carbocyclic ring or heterocyclic ring is optionally independently substituted with one or more substituents independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹,

wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;

each of R¹ and R² that is not taken together with the atoms connecting them to form one ring is independently selected from:

C₁-C₆ alkyl, C₁-C₆ haloalkyl, C₁-C₆ alkoxy, C₁-C₆ haloalkoxy, halo, CN, NO₂, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NR⁸R⁹, C(O)R¹³, CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,

wherein the C₁-C₆ alkyl, C₁-C₆ alkoxy, C₁-C₆ haloalkyl, C₆-C₁₀ aryl, 5- to 10-membered heteroaryl, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, R¹⁵, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);

wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, —O(C₀-C₃ alkylene)C₆-C₁₀ aryl, halo, NR⁸R⁹, or oxo;

wherein the 3- to 7-membered heterocycloalkyl, C₆-C₁₀ aryl, and 5- to 10-membered heteroaryl are optionally substituted with one or more substituents independently selected from halo, C₁-C₆ alkyl, and OC₁-C₆ alkyl;

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, C₂-C₆ alkenyl, and C₂-C₆ alkynyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, C₃-C₁₀ cycloalkyl, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), C₆-C₁₀ aryloxy, C₃-C₁₀ cycloalkoxy, and S(O₂)C₁-C₆ alkyl; and

wherein the C₁-C₆ alkyl or C₁-C₆ alkoxy that R⁶ or R⁷ is substituted with is optionally substituted with one or more hydroxyl, C₆-C₁₀ aryl, or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;

wherein the 3- to 7-membered heterocycloalkyl, C₆-C₁₀ aryl, and 5- to 10-membered heteroaryl are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

each of R⁴ and R⁵ is independently selected from hydrogen and C₁-C₆ alkyl;

R¹⁰ is C₁-C₆ alkyl;

each of R⁸ and R⁹ at each occurrence is independently selected from hydrogen, C₁-C₆ alkyl, C₂-C₆ alkenyl, C₃-C₇ cycloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl, 3- to 7-membered heterocycloalkyl, or NR¹¹R¹²;

or R⁸ and R⁹ taken together with the nitrogen they are attached to form a 3- to 10-membered monocyclic or bicyclic ring optionally containing one or more heteroatoms in addition to the nitrogen they are attached to, wherein the ring is optionally substituted with one or more substituents independently selected from halo, C₁-C₆ alkyl, C₁-C₆ haloalkyl, C₁-C₆ alkoxy, oxo, N(C₁-C₆ alkyl)₂, NH₂, NH(C₁-C₆ alkyl), and hydroxy;

R¹³ is C₁-C₆ alkyl or —(Z¹-Z²)ₐ₁—Z³;

each of R¹¹ and R¹² at each occurrence is independently selected from hydrogen, C₁-C₆ alkyl, and —(Z¹-Z²)ₐ₁—Z³;

a1 is 0-10 (e.g., 0-4);

each Z¹ is independently C₁-C₆ alkylene optionally substituted with one or more substituents independently selected from oxo, halo, and hydroxy;

each Z² is independently a bond, NH, N(C₁-C₆ alkyl), —O—, —S—, or 5-10 membered heteroarylene;

Z³ is independently C₆-C₁₀ aryl, C₂-C₆ alkyenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆ alkyl)₂, NH₂, NH(C₁-C₆ alkyl), and hydroxy;

R³ is selected from hydrogen, cyano, hydroxy, C₁-C₆ alkoxy, C₁-C₆ alkyl, and

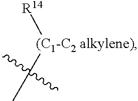

wherein the C₁-C₂ alkylene group is optionally substituted by oxo;

R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally substituted with from 1-3 independently selected R⁶,

R¹⁵ is —(Z⁴-Z⁵)ₐ₂—Z⁶;

a2 is an integer selected from 1-10 (e.g., 1-5 (e.g., 2-5));

each Z⁴ is independently selected from —O—, —S—, —NH—, and —N(C₁-C₃ alkyl)-;

provided that the Z⁴ group directly attached to R¹ or R² is —O— or —S—;

each Z⁵ is independently C₁-C₆ alkylene optionally substituted with one or more substituents independently selected from oxo, halo, and hydroxy; and

Z⁶ is OH, OC₁-C₆ alkyl, NH₂, NH(C₁-C₆ alkyl), N(C₁-C₆ alkyl)₂, NHC(O)(C₁-C₆ alkyl), NHC(O)(C₁-C₆ alkoxy), or an optionally substituted group selected from the group consisting of:

C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆ alkyl)₂, NH₂, NH(C₁-C₆ alkyl), and hydroxy;

or a pharmaceutically acceptable salt thereof.

In another aspect, provided herein is a compound of Formula AA

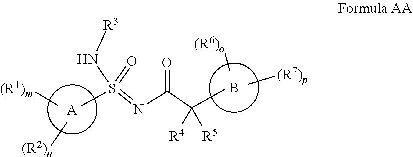

wherein

m=0, 1, or 2;

n=0, 1, or 2;

o=1 or 2;

p=0, 1, 2, or 3;

wherein

A is a 5-10-membered monocyclic or bicyclic heteroaryl or a C₆-C₁₀ monocyclic or bicyclic aryl;

B is a 5-10-membered monocyclic or bicyclic heteroaryl or a C₆-C₁₀ monocyclic or bicyclic aryl;

wherein

at least one R⁶ is ortho to the bond connecting the B ring to the C(R⁴R⁵) group of Formula AA;

one pair of R¹ and R² are on adjacent atoms, taken together with the atoms connecting them, independently form one monocyclic or bicyclic C₄-C₁₂ carbocyclic ring or one monocyclic or bicyclic 5-to-12-membered heterocyclic ring containing 1-3 heteroatoms independently selected from O, N, and S, wherein the carbocyclic ring or heterocyclic ring is optionally independently substituted with one or more substituents independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹,

wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;

each of R¹ and R² that is not taken together with the atoms connecting them to form one ring is independently selected from:

C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO—C₆-C₁₀ aryl, CO-5- to 10-membered heteroaryl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,

wherein the C₁-C₆ alkyl, C₁-C₆ alkoxy, C₁-C₆ haloalkyl, C₆-C₁₀ aryl, 5- to 10-membered heteroaryl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, R¹⁵, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;

wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, —O(C₀-C₃ alkylene)C₆-C₁₀ aryl, halo, NR⁸R⁹, or oxo;

wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, C₂-C₆ alkenyl, and C₂-C₆ alkynyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, C₃-C₁₀ cycloalkyl, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, C₃-C₁₀ cycloalkoxy, and S(O₂)C₁-C₆ alkyl; and

wherein the C₁-C₆ alkyl or C₁-C₆ alkoxy that R⁶ or R⁷ is substituted with is optionally substituted with one or more hydroxyl, C₆-C₁₀ aryl, or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;

wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

each of R⁴ and R⁵ is independently selected from hydrogen and C₁-C₆ alkyl;

R¹⁰ is C₁-C₆ alkyl;

each of R⁸ and R⁹ at each occurrence is independently selected from hydrogen, C₁-C₆ alkyl, C₂-C₆ alkenyl, C₃-C₇ cycloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;

R¹³ is C₁-C₆ alkyl, C₆-C₁₀ aryl, or 5- to 10-membered heteroaryl;

each of R¹¹ and R¹² at each occurrence is independently selected from hydrogen and C₁-C₆ alkyl;

R³ is selected from hydrogen, cyano, hydroxy, C₁-C₆ alkoxy, C₁-C₆ alkyl, and

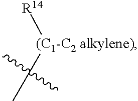

wherein the C₁-C₂ alkylene group is optionally substituted by oxo;

R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally independently substituted with from 1-3 R⁶,

R¹⁵ is —(Z⁴-Z⁵)ₐ₂—Z⁶;

a2 is an integer selected from 1-10 (e.g., 1-5 (e.g., 2-5));

each Z⁴ is independently selected from —O—, —S—, —NH—, and —N(C₁-C₃ alkyl)-;

provided that the Z⁴ group directly attached to R¹ or R² is —O— or —S—;

each Z⁵ is independently C₁-C₆ alkylene optionally substituted with one or more substituents independently selected from oxo, halo, and hydroxy; and

Z⁶ is OH, OC₁-C₆ alkyl, NH₂, NH(C₁-C₆ alkyl), N(C₁-C₆ alkyl)₂, NHC(O)(C₁-C₆ alkyl), NHC(O)(C₁-C₆ alkoxy), or an optionally substituted group selected from the group consisting of:

C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆ alkyl)₂, NH₂, NH(C₁-C₆ alkyl), and hydroxy;

or a pharmaceutically acceptable salt thereof.

In certain embodiments of any of the foregoing, the compound is other than:

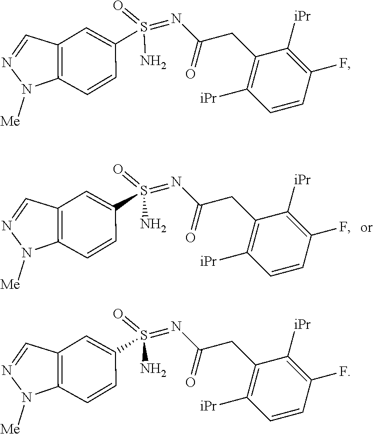 [SMILES: CC(C)c1ccc(F)c(C(C)C)c1CC(=O)N=S(N)(=O)c1ccc2c(cnn2C)c1 ; CC(C)c1ccc(F)c(C(C)C)c1CC(=O)N=[S@](N)(=O)c1ccc2c(cnn2C)c1 ; CC(C)c1ccc(F)c(C(C)C)c1CC(=O)N=[S@@](N)(=O)c1ccc2c(cnn2C)c1]

In some embodiments, provided herein is a compound of Formula AA:

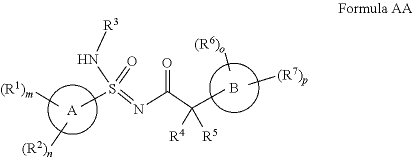

wherein

m=0, 1, or 2;

n=0, 1, or 2;

o=1 or 2;

p=0, 1, 2, or 3;

wherein

A is a 5-10-membered monocyclic or bicyclic heteroaryl or a C₆-C₁₀ monocyclic or bicyclic aryl;

B is a 5-10-membered monocyclic or bicyclic heteroaryl or a C₆-C₁₀ monocyclic or bicyclic aryl;

wherein

at least one R⁶ is ortho to the bond connecting the B ring to the C(R⁴R⁵) group of Formula AA;

R¹ and R² are each independently selected from C₁-C₆ alkyl, C₁-C₆ haloalkyl, C₁-C₆ alkoxy, C₁-C₆ haloalkoxy, halo, CN, NO₂, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NR⁸R⁹, NH—(C═NR¹³)NR¹¹R¹², C(O)R¹³, CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,

wherein the C₁-C₆ alkyl, C₁-C₆ alkoxy, C₁-C₆ haloalkyl, C₆-C₁₀ aryl, 5- to 10-membered heteroaryl, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, R¹⁵, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);

wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, —O(C₀-C₃ alkylene)C₆-C₁₀ aryl, halo, NR⁸R⁹, or oxo;

wherein the 3- to 7-membered heterocycloalkyl, C₆-C₁₀ aryl, and 5- to 10-membered heteroaryl are optionally substituted with one or more substituents independently selected from halo, C₁-C₆ alkyl, and OC₁-C₆ alkyl;

or one pair of R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring containing 1-3 heteroatoms independently selected from O, N, and S, wherein the carbocyclic ring or heterocyclic ring is optionally independently substituted with one or more substituents independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹,

wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;

provided that:

(1) one or more of R¹ or R², when present, is selected from NR^{8′}R^{9′}, C(O)NR^{8′}R^{9′}, NH—C(═NR^{13′})NR^{11′}R^{12′}, S(O)₂NR^{11′}R^{12′}, C(O)R^{13′}, C₁-C₆ alkyl, C₁-C₆ haloalkyl, C₃-C₇ cycloalkyl, 3- to 7-membered heterocycloalkyl, C₁-C₆ alkoxy, C₆-C₁₀ aryl, and 5- to 10-membered heteroaryl,

wherein each of the C₃-C₇ cycloalkyl and 3- to 7-membered heterocycloalkyl is substituted with R^{15′}, NR^{8′}R^{9′} or C(O)NR^{8′}R^{9′};

each of the C₁-C₆ alkyl and C₁-C₆ haloalkyl is substituted with R¹⁵, NR^{8′}R^{9′}, or C(O)NR^{8′}R^{9′};

each of the C₆-C₁₀ aryl and 5- to 10-membered heteroaryl is substituted with one or more substituents each independently selected from hydroxy, halo, CN, oxo, C₁-C₆ alkyl, C₁-C₆ alkoxy, R¹⁵, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl); or

(2) one pair of R¹ and R² on adjacent atoms, taken together with the atoms connecting them, independently form one ring that is selected from:

(a) C₄-C₈ carbocyclic ring or 5-to-8-membered heterocyclic ring containing 1 or 2 heteroatoms independently selected from O, N, and S, wherein the carbocyclic ring or heterocyclic ring is independently substituted with one or more substituents each independently selected from from C₂-C₆ alkenyl, C₂-C₆ alkynyl, OC₃-C₁₀ cycloalkyl, CN, OS(O₂)C₆-C₁₀ aryl, S(O₂)C₆-C₁₀ aryl, 5- to 10-membered heteroaryl, C₃-C₁₀ cycloalkyl, and 3- to 10-membered heterocycloalkyl, wherein the S(O₂)C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;

(b) C₄-C₈ carbocyclic ring or 5-to-8-membered heterocyclic ring containing 3 heteroatoms independently selected from O, N, and S, wherein the carbocyclic ring or heterocyclic ring is optionally independently substituted with one or more substituents independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹; and

(c) monocyclic or bicyclic C₉-C₁₂ carbocyclic ring or monocyclic or bicyclic 9- to 12-membered heterocyclic ring containing 1-3 heteroatoms independently selected from O, N, and S, wherein the carbocyclic ring or heterocyclic ring is optionally independently substituted with one or more substituents independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, C₂-C₆ alkenyl, and C₂-C₆ alkynyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, C₃-C₁₀ cycloalkyl, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), C₆-C₁₀ aryloxy, C₃-C₁₀ cycloalkoxy, and S(O₂)C₁-C₆ alkyl; and

wherein the C₁-C₆ alkyl or C₁-C₆ alkoxy that R⁶ or R⁷ is substituted with is optionally substituted with one or more hydroxyl, C₆-C₁₀ aryl, or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;

wherein the 3- to 7-membered heterocycloalkyl, C₆-C₁₀ aryl, and 5- to 10-membered heteroaryl are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

each of R⁴ and R⁵ is independently selected from hydrogen and C₁-C₆ alkyl;

R¹⁰ is C₁-C₆ alkyl;

each of R⁸ and R⁹ at each occurrence is independently selected from hydrogen, C₁-C₆ alkyl, C₂-C₆ alkenyl, C₃-C₇ cycloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl, 3- to 7-membered heterocycloalkyl, or NR¹¹R¹²

or R⁸ and R⁹ taken together with the nitrogen they are attached to form a 3- to 10-membered monocyclic or bicyclic ring optionally containing one or more heteroatoms in addition to the nitrogen they are attached to, wherein the ring is optionally substituted with one or more substituents independently selected from halo, C₁-C₆ alkyl, C₁-C₆ haloalkyl, C₁-C₆ alkoxy, oxo, N(C₁-C₆ alkyl)₂, NH₂, NH(C₁-C₆ alkyl), and hydroxy;

each of R^{8′} and R^{9′} at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl, 3- to 7-membered heterocycloalkyl, or NR¹¹R¹²;

or R^{8′} and R^{9′} taken together with the nitrogen they are attached to form a 3- to 10-membered monocyclic or bicyclic ring optionally containing one or more heteroatoms in addition to the nitrogen they are attached to, wherein the ring is optionally substituted with one or more substituents independently selected from halo, C₁-C₆ alkyl, C₁-C₆ haloalkyl, C₁-C₆ alkoxy, oxo, N(C₁-C₆ alkyl)₂, NH₂, NH(C₁-C₆ alkyl), and hydroxy;

provided that:

(1) one or more occurrences of R^{8′} or R^{9′} is C₁-C₆ alkyl, C₂-C₆ alkenyl, C₃-C₇ cycloalkyl, (C═NR^{13′})NR^{11′}R^{12′}, S(O₂)NR^{11′}R^{12′}, C(O)R^{13′}, CO₂R^{13′} and CONR^{11′}R^{12′}; wherein the C₁-C₆ alkyl is substituted with NR¹¹R¹²;

(2) one or more pairs of R^{8′} and R^{9′} attached to the same nitrogen taken together with the nitrogen they are attached to form:

(a) a 8- to 10-membered monocyclic or bicyclic ring optionally containing one or more heteroatoms in addition to the nitrogen they are attached to, wherein the ring is optionally substituted with one or more substituents independently selected from halo, C₁-C₆ alkyl, C₁-C₆ haloalkyl, C₁-C₆ alkoxy, oxo, N(C₁-C₆ alkyl)₂, NH₂, NH(C₁-C₆ alkyl), and hydroxy; or

(b) a 3- to 7-membered monocyclic or bicyclic ring optionally containing one or more heteroatoms in addition to the nitrogen they are attached to, wherein the ring is substituted with one or more substituents independently selected from halo, C₁-C₆ alkyl, C₁-C₆ haloalkyl, C₁-C₆ alkoxy, oxo, N(C₁-C₆ alkyl)₂, NH₂, NH(C₁-C₆ alkyl), and hydroxy;

R¹³ is C₁-C₆ alkyl or —(Z¹-Z²)ₐ₁—Z³

R^{13′} is —(Z¹-Z²)ₐ₁—Z^{3′};

each of R¹¹ and R¹² at each occurrence is independently selected from hydrogen, C₁-C₆ alkyl, and —(Z¹-Z²)ₐ₁—Z³;

each of R^{11′} and R^{12′} at each occurrence is independently selected from hydrogen, C₁-C₆ alkyl, and —(Z¹-Z²)ₐ₁—Z³,

provided that one or more occurrences of R^{11′} and R^{12′} is —(Z¹-Z²)ₐ₁—Z³;

a1 is 0, 1, 2, 3, or 4;

each Z¹ is independently C₁-C₆ alkylene optionally substituted with one or more substituents independently selected from oxo, halo, and hydroxy;

each Z² is independently a bond, NH, N(C₁-C₆ alkyl), —O—, —S—, or 5-10 membered heteroarylene;

Z³ is independently C₆-C₁₀ aryl, C₂-C₆ alkyenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆ alkyl)₂, NH₂, NH(C₁-C₆ alkyl), and hydroxy;

when a1 is 0, Z^{3′} is independently C₆₋₁₀ aryl, C₃-C₁₀ cycloalkyl, 5- to 10-membered heteroaryl, 3- to 10-membered heterocycloalkyl, C₂-C₆ alkenyl, or C₂-C₆ alkynyl, each of which is substituted with one or more substituents independently selected from halo, C₁-C₆ alkyl, C₁₋₆ haloalkyl, C₁-C₆ alkoxy, oxo, N(C₁-C₆ alkyl)₂, NH₂, NH(C₁-C₆ alkyl), and hydroxy;

when a1 is 1-10, Z^{3′} is an independently selected Z³;

R³ is selected from hydrogen, cyano, hydroxy, C₁-C₆ alkoxy, C₁-C₆ alkyl, and

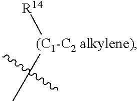

wherein the C₁-C₂ alkylene group is optionally substituted by oxo;

R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally substituted with from 1-3 independently selected R⁶,

R¹⁵ is —(Z⁴-Z⁵)ₐ₂—Z⁶;

R^{15′} is —(Z⁴-Z⁵)_{a2′}—Z⁶;

a2 is an integer selected from 1-10 (e.g., 1-5);

a2′ is an integer selected from 2-10 (e.g., 2-5);

each Z⁴ is independently selected from —O—, —S—, —NH—, and —N(C₁-C₃ alkyl)-;

provided that the Z⁴ group directly attached to R¹ or R² is —O— or —S—;

each Z⁵ is independently C₁-C₆ alkylene optionally substituted with one or more substituents independently selected from oxo, halo, and hydroxy; and

Z⁶ is OH, OC₁-C₆ alkyl, NH₂, NH(C₁-C₆ alkyl), N(C₁-C₆ alkyl)₂, NHC(O)(C₁-C₆ alkyl), NHC(O)(C₁-C₆ alkoxy), or an optionally substituted group selected from the group consisting of:

C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆ alkyl)₂, NH₂, NH(C₁-C₆ alkyl), and hydroxy

or a pharmaceutically acceptable salt thereof.

In some embodiments, provided herein is a compound of Formula AA

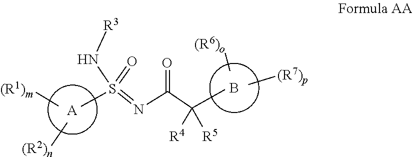

wherein
m=0, 1, or 2
n=0, 1, or 2
o=1 or 2
p=0, 1, 2, or 3
wherein
A is a 5-10-membered monocyclic or bicyclic heteroaryl or a C₆-C₁₀ monocyclic or bicyclic aryl;
B is a 5-10-membered monocyclic or bicyclic heteroaryl or a C₆-C₁₀ monocyclic or bicyclic aryl;
wherein
at least one R⁶ is ortho to the bond connecting the B ring to the C(R⁴R⁵) group of Formula AA;
R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO—C₆-C₁₀ aryl, CO-5- to 10-membered heteroaryl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₁₀ cycloalkyl and 3- to 10-membered heterocycloalkyl, and a C₂-C₆ alkenyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo; wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹, wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
   R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
   wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen and optionally substituted with one or more halo, OH, oxo, or C₁-C₆ alkyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹′ wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
   each of R⁴ and R⁵ is independently selected from hydrogen and C₁-C₆ alkyl;
   R¹⁰ is C₁-C₆ alkyl;
   each of R⁸ and R⁹ at each occurrence is independently selected from hydrogen, C₁-C₆ alkyl, NH—(C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to; R¹³ is C₁-C₆ alkyl, C₆-C₁₀ aryl, or 5- to 10-membered heteroaryl;
   each of R¹¹ and R¹² at each occurrence is independently selected from hydrogen and C₁-C₆ alkyl;
   R³ is selected from hydrogen, cyano, hydroxy, C₁-C₆ alkoxy, C₁-C₆ alkyl, and

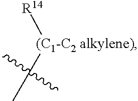

wherein the C₁-C₂ alkylene group is optionally substituted by oxo;
R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally independently substituted with 1, 2, or 3 R⁶
or a pharmaceutically acceptable salt thereof.

In some embodiments, provided herein is a compound of Formula AA

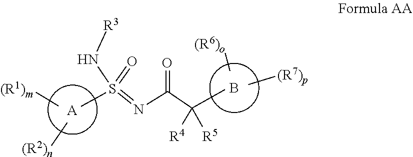

wherein
m=0, 1, or 2
n=0, 1, or 2
o=1 or 2
p=0, 1, 2, or 3
wherein
A is a 5-10-membered monocyclic or bicyclic heteroaryl or a C₆-C₁₀ monocyclic or bicyclic aryl;
B is a 5-10-membered monocyclic or bicyclic heteroaryl or a C₆-C₁₀ monocyclic or bicyclic aryl;
wherein
at least one R⁶ is ortho to the bond connecting the B ring to the C(R⁴R⁵) group of Formula AA; R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO—C₆-C₁₀ aryl, CO-5- to 10-membered heteroaryl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₁₀ cycloalkyl and 3- to 10-membered heterocycloalkyl, and a C₂-C₆ alkenyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo; wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹, wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
   R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
   wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen and optionally substituted with one or more halo, OH, oxo, or C₁-C₆ alkyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R9′ wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
   each of R⁴ and R⁵ is independently selected from hydrogen and C₁-C₆ alkyl;
   R¹⁰ is C₁-C₆ alkyl;
   each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to; R¹³ is C₁-C₆ alkyl, C₆-C₁₀ aryl, or 5- to 10-membered heteroaryl;
   each of R¹¹ and R¹² at each occurrence is independently selected from hydrogen and C₁-C₆ alkyl;
   R³ is selected from hydrogen, cyano, hydroxy, C₁-C₆ alkoxy, C₁-C₆ alkyl, and

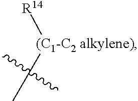

wherein the C₁-C₂ alkylene group is optionally substituted by oxo;
R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally independently substituted with 1, 2, or 3 R⁶
or a pharmaceutically acceptable salt thereof.

In some embodiments, provided herein is a compound of Formula AA

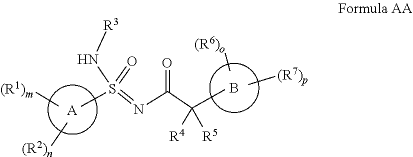

wherein
m=0, 1 or 2
n=0, 1 or 2
o=1 or 2
p=0, 1, 2 or 3
wherein
A is a 5-10-membered monocyclic or bicyclic heteroaryl or a C₆-C₁₀ monocyclic or bicyclic aryl;
B is a 5-10-membered monocyclic or bicyclic heteroaryl or a C₆-C₁₀ monocyclic or bicyclic aryl;
wherein
at least one R⁶ is ortho to the bond connecting the B ring to the C(R⁴R⁵) group of Formula AA;
R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO-5- to 10-membered heteroaryl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo; wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹′ wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
   R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and a C₂-C₆ alkenyl,
   wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
   each of R⁴ and R⁵ is independently selected from hydrogen and C₁-C₆ alkyl;
   R¹⁰ is C₁-C₆ alkyl;
   each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;
   R¹³ is C₁-C₆ alkyl, C₆-C₁₀ aryl, or 5- to 10-membered heteroaryl;
   each of R¹¹ and R¹² at each occurrence is independently selected from hydrogen and C₁-C₆ alkyl; and
   R³ is selected from hydrogen, cyano, hydroxy, C₁-C₆ alkoxy, C₁-C₆ alkyl, CO₂C₁-C₆ alkyl, and

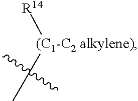

wherein the C₁-C₂ alkylene group is optionally substituted by oxo; R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally independently substituted with 1 or 2 R⁶
or a pharmaceutically acceptable salt thereof.

In some embodiments, provided herein is a compound of Formula AA

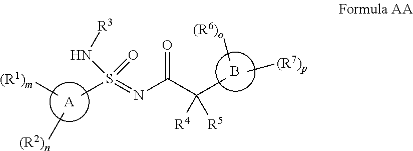

wherein
m=0, 1, or 2;
n=0, 1, or 2;
o=1 or 2;
p=0, 1, 2, or 3;
wherein
A is a 5-10-membered heteroaryl or a C₆-C₁₀ aryl;
B is a 5-10-membered heteroaryl or a C₆-C₁₀ aryl;
wherein
at least one R⁶ is ortho to the bond connecting the B ring to the C(R⁴R⁵) group of Formula AA;
R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO—C₆-C₁₀ aryl, CO-5- to 10-membered heteroaryl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₁₀ cycloalkyl, 3- to 10-membered heterocycloalkyl, and C₂-C₆ alkenyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo; wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) of the R¹ or R² C₁-C₆ alkyl, the R¹ or R² C₁-C₆ haloalkyl, the R¹ or R² C₃-C₇ cycloalkyl, or the R¹ or R² 3- to 7-membered heterocycloalkyl are optionally substituted with one or more substituents independently selected from halo, C₁-C₆ alkyl, and OC₁-C₆ alkyl;
   or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹, wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
   R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl, 3- to 7-membered heterocycloalkyl, and C₂-C₆ alkenyl,
   wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxy, halo, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹′ wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
   each of R⁴ and R⁵ is independently selected from hydrogen and C₁-C₆ alkyl;
   R¹⁰ is C₁-C₆ alkyl;
   each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;
   R¹³ is C₁-C₆ alkyl, C₆-C₁₀ aryl, or 5- to 10-membered heteroaryl;
   each of R¹¹ and R¹² at each occurrence is independently selected from hydrogen and C₁-C₆ alkyl;
   R³ is selected from hydrogen, cyano, hydroxy, C₁-C₆ alkoxy, C₁-C₆ alkyl, and

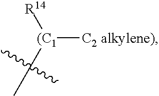

wherein the C₁-C₂ alkylene group is optionally substituted by oxo;
and
R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally independently substituted with 1, 2, or 3 R⁶;
or a pharmaceutically acceptable salt thereof.

In some embodiments, provided herein is a compound of Formula AA

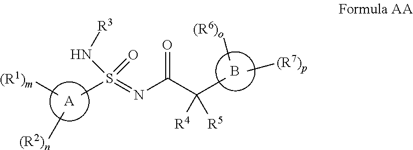

wherein
m=0, 1, or 2;
n=0, 1, or 2;
o=1 or 2;
p=0, 1, 2, or 3;
wherein
A is a 5-10-membered monocyclic or bicyclic heteroaryl or a C₆-C₁₀ monocyclic or bicyclic aryl;
B is a 5-10-membered monocyclic or bicyclic heteroaryl or a C₆-C₁₀ monocyclic or bicyclic aryl;
wherein
at least one R⁶ is ortho to the bond connecting the B ring to the C(R⁴R⁵) group of Formula AA;
R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO-5- to 10-membered heteroaryl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) of the R¹ or R² C₁-C₆ alkyl, the R¹ or R² C₁-C₆ haloalkyl, the R¹ or R² C₃-C₇ cycloalkyl, or the R¹ or R² 3- to 7-membered heterocycloalkyl are optionally substituted with one or more substituents independently selected from halo, C₁-C₆ alkyl, and OC₁-C₆ alkyl;
   or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹, wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
   R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and C₂-C₆ alkenyl,
   wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
   each of R⁴ and R⁵ is independently selected from hydrogen and C₁-C₆ alkyl;
   R¹⁰ is C₁-C₆ alkyl;
   each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or
   R⁸ and R⁹ taken together with the nitrogen they are attached to form a 3- to 7-membered ring optionally containing one or more heteroatoms in addition to the nitrogen they are attached to;
   R¹³ is C₁-C₆ alkyl, C₆-C₁₀ aryl, or 5- to 10-membered heteroaryl;
   each of R¹¹ and R¹² at each occurrence is independently selected from hydrogen and C₁-C₆ alkyl; and
   R³ is selected from hydrogen, cyano, hydroxy, C₁-C₆ alkoxy, C₁-C₆ alkyl, CO₂C₁-C₆ alkyl, and

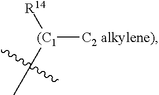

wherein the C₁-C₂ alkylene group is optionally substituted by oxo;
R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally independently substituted with 1 or 2 R⁶;
or a pharmaceutically acceptable salt thereof.

Provided herein is a compound of Formula AA

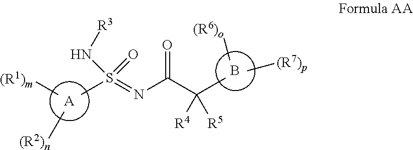

wherein
m=0, 1, or 2;
n=0, 1, or 2;
o=1 or 2;
p=0, 1, 2, or 3;
wherein
A is a 5-10-membered monocyclic or bicyclic heteroaryl or a C₆-C₁₀ monocyclic or bicyclic aryl;
B is a 5-10-membered monocyclic or bicyclic heteroaryl or a C₆-C₁₀ monocyclic or bicyclic aryl;
wherein
at least one R⁶ is ortho to the bond connecting the B ring to the C(R⁴R⁵) group of Formula AA;
R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO-5- to 10-membered heteroaryl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo; wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) of the R¹ or R² C₁-C₆ alkyl, the R¹ or R² C₁-C₆ haloalkyl, the R¹ or R² C₃-C₇ cycloalkyl, or the R¹ or R² 3- to 7-membered heterocycloalkyl are optionally substituted with one or more substituents independently selected from halo, C₁-C₆ alkyl, and OC₁-C₆ alkyl;
   or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹, wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
   R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and C₂-C₆ alkenyl,
   wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
   each of R⁴ and R⁵ is independently selected from hydrogen and C₁-C₆ alkyl;
   R¹⁰ is C₁-C₆ alkyl;
   each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;
   R¹³ is C₁-C₆ alkyl, C₆-C₁₀ aryl, or 5- to 10-membered heteroaryl;
   each of R¹¹ and R¹² at each occurrence is independently selected from hydrogen and C₁-C₆ alkyl; and
   R³ is selected from hydrogen, cyano, hydroxy, C₁-C₆ alkoxy, C₁-C₆ alkyl, CO₂C₁-C₆ alkyl, and

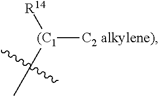

wherein the C₁-C₂ alkylene group is optionally substituted by oxo;
R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally independently substituted with 1 or 2 R⁶;
or a pharmaceutically acceptable salt thereof.

In some embodiments, provided herein is a compound of Formula AA

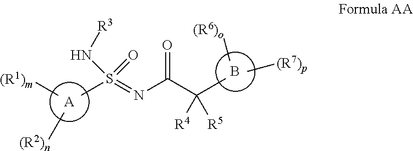

wherein
m=0, 1, or 2;
n=0, 1, or 2;
o=1 or 2;
p=0, 1, 2, or 3;
wherein
A is a 5-10-membered monocyclic or bicyclic heteroaryl or a C₆-C₁₀ monocyclic or bicyclic aryl;
B is a 5-10-membered monocyclic or bicyclic heteroaryl or a C₆-C₁₀ monocyclic or bicyclic aryl;
wherein
at least one R⁶ is ortho to the bond connecting the B ring to the C(R⁴R⁵) group of Formula AA;
R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO—C₆-C₁₀ aryl, CO-5- to 10-membered heteroaryl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₁₀ cycloalkyl and 3- to 10-membered heterocycloalkyl, and a C₂-C₆ alkenyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹′ wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen; wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹, wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
each of R⁴ and R⁵ is independently selected from hydrogen and C₁-C₆ alkyl;
R¹⁰ is C₁-C₆ alkyl;
each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or
R⁸ and R⁹ taken together with the nitrogen they are attached to form a 3- to 7-membered ring optionally containing one or more heteroatoms in addition to the nitrogen they are attached to;
R¹³ is C₁-C₆ alkyl, C₆-C₁₀ aryl, or 5- to 10-membered heteroaryl;
each of R¹¹ and R¹² at each occurrence is independently selected from hydrogen and C₁-C₆ alkyl;
R³ is selected from hydrogen, cyano, hydroxy, C₁-C₆ alkoxy, C₁-C₆ alkyl, and

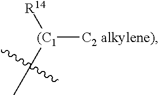

wherein the C₁-C₂ alkylene group is optionally substituted by oxo;
R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally independently substituted with 1, 2, or 3 R⁶
or a pharmaceutically acceptable salt thereof.

In any of the embodiments described herein, the compound of Formula AA is not one of the following:

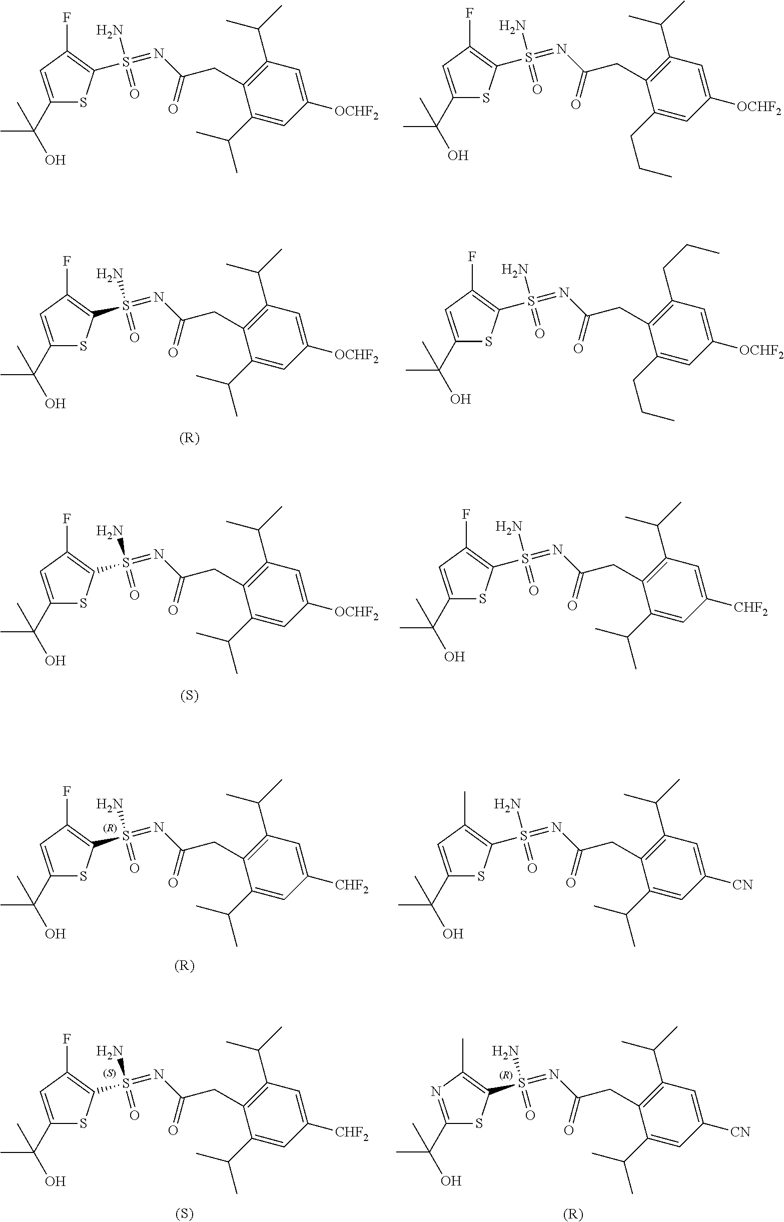

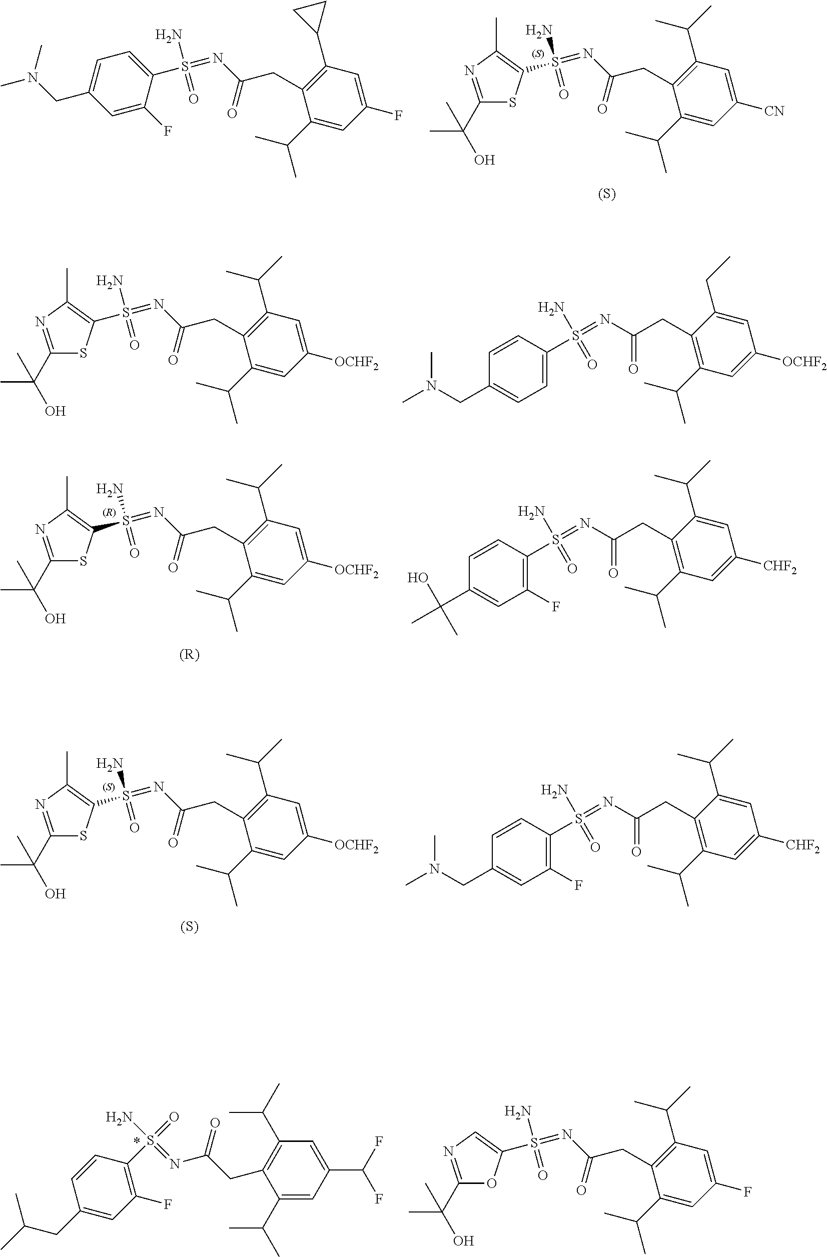

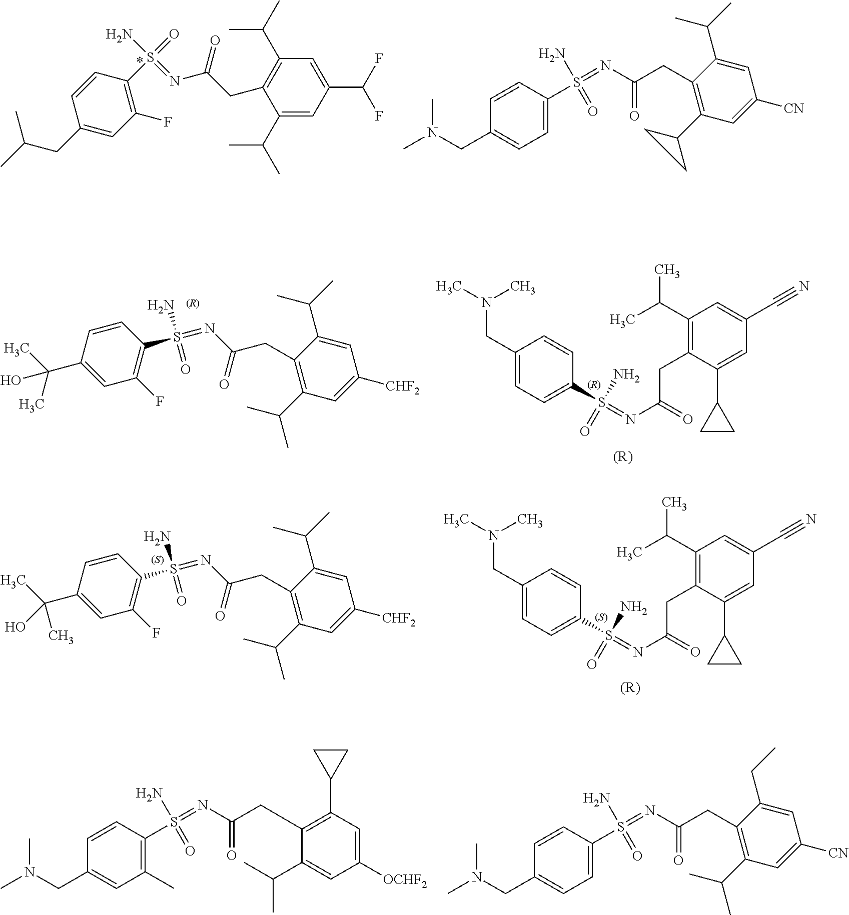

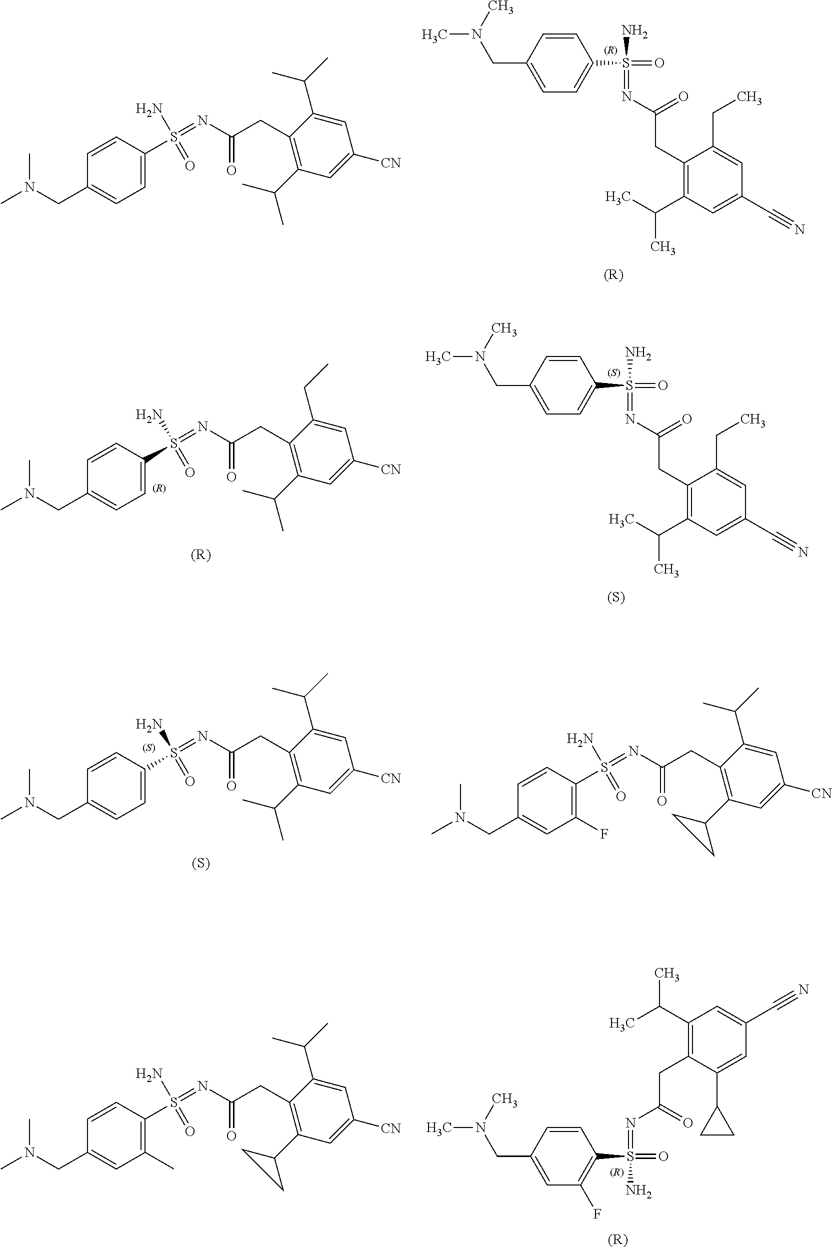

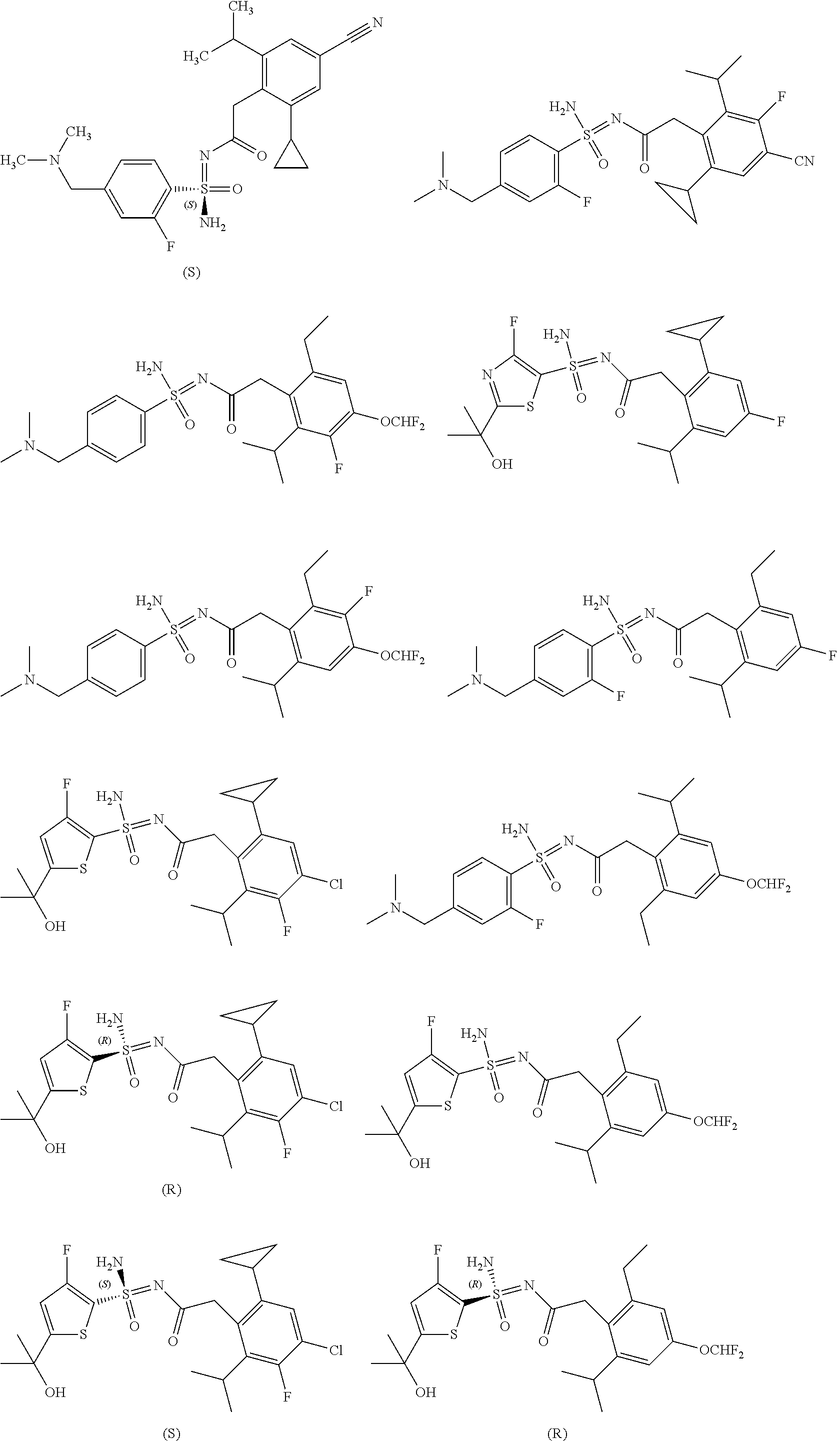

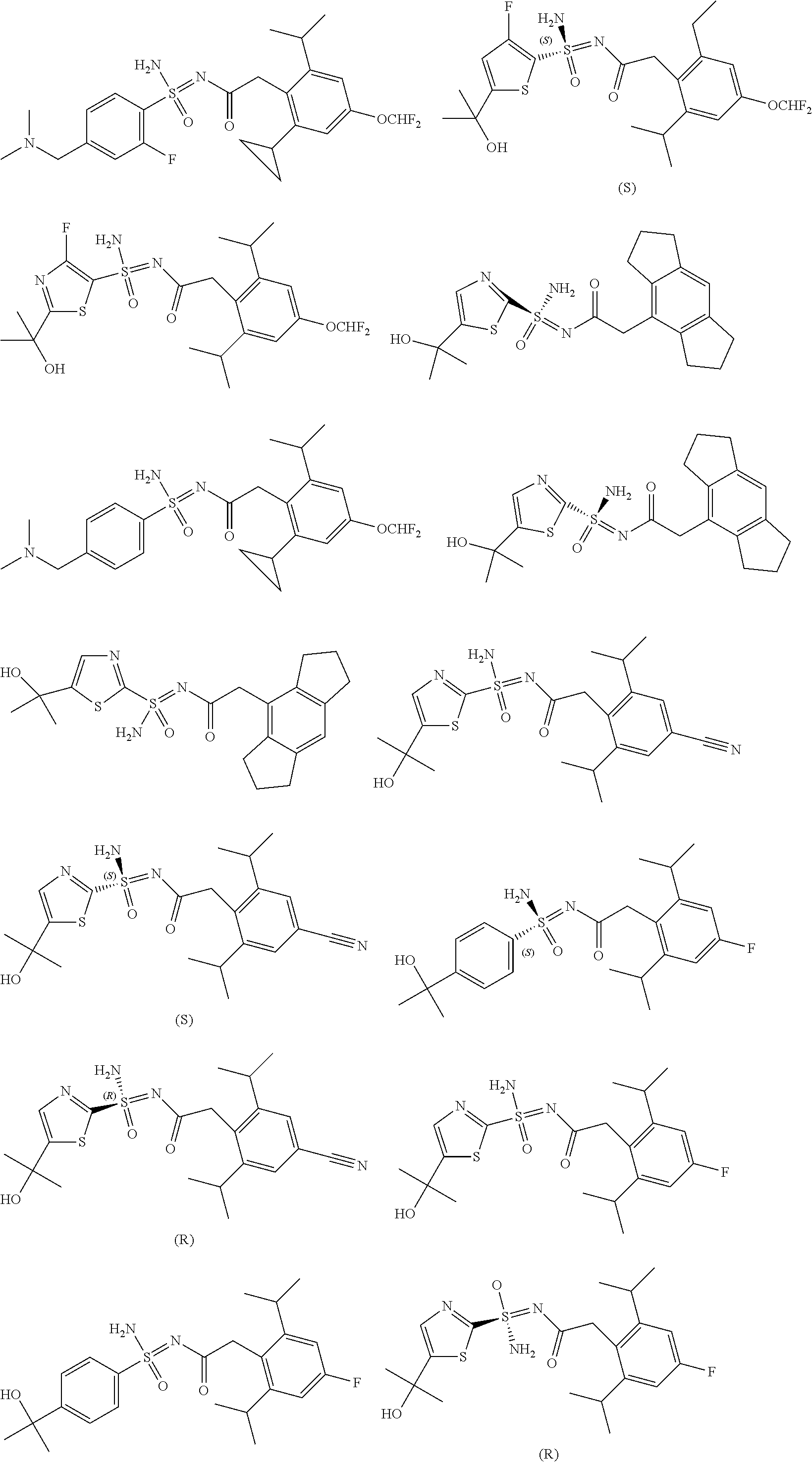

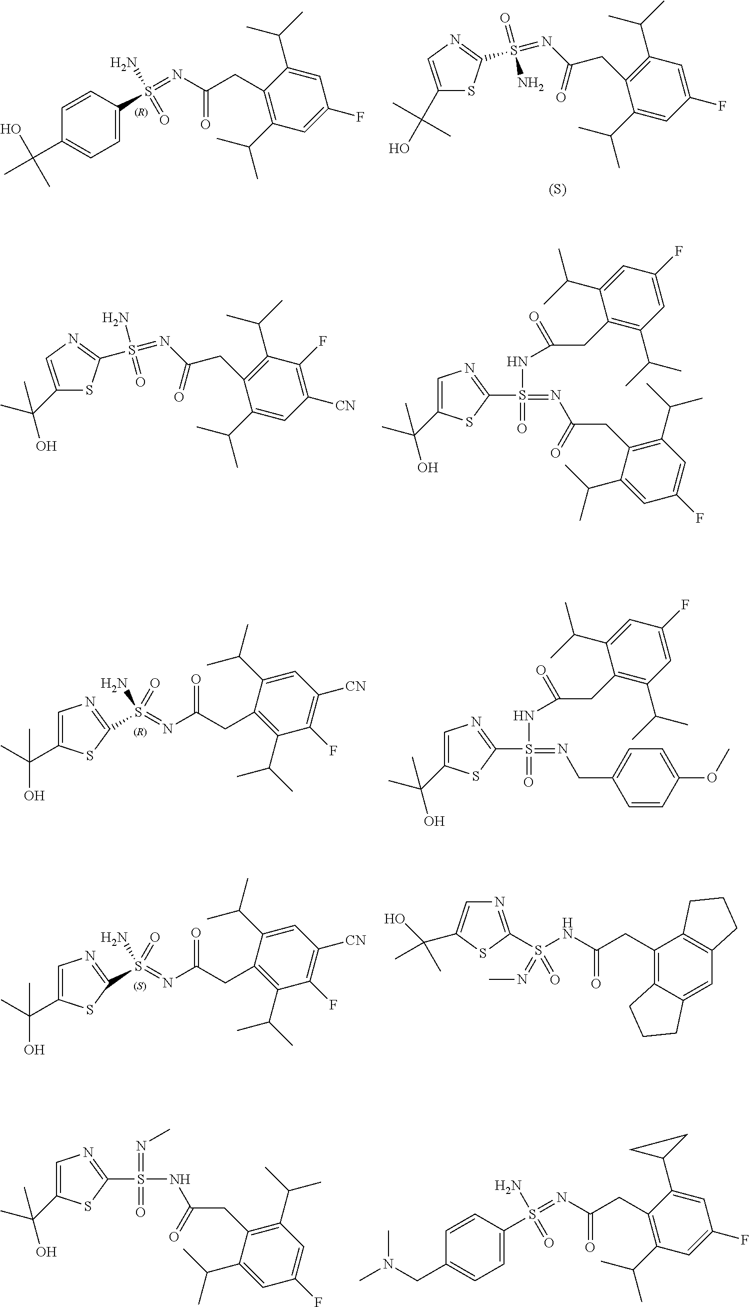

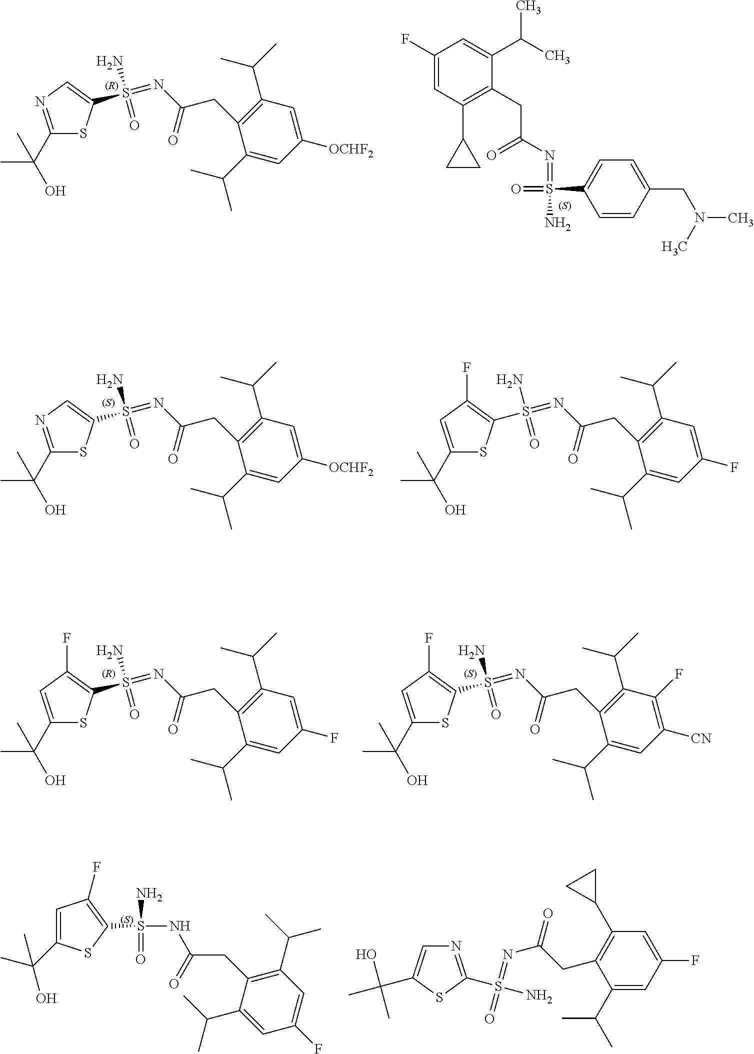

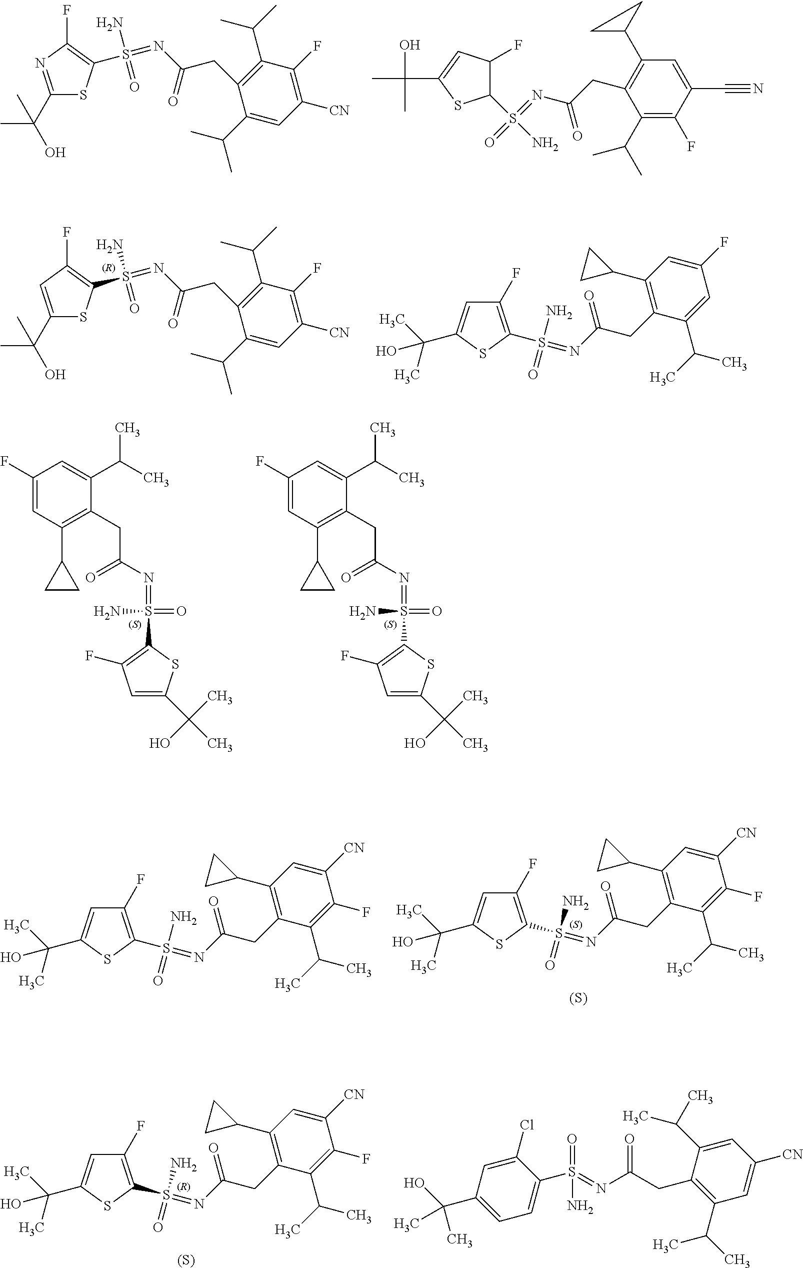

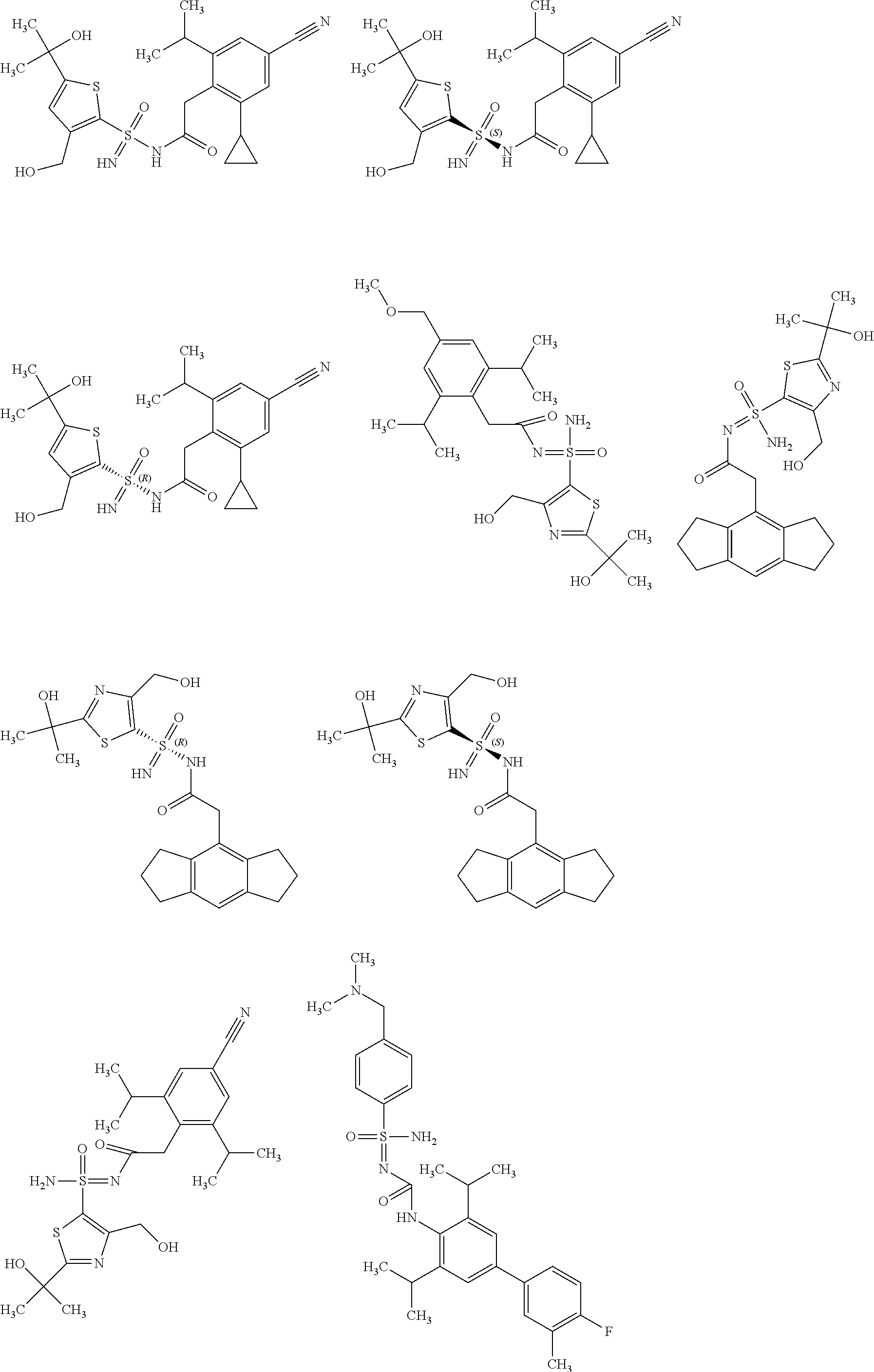

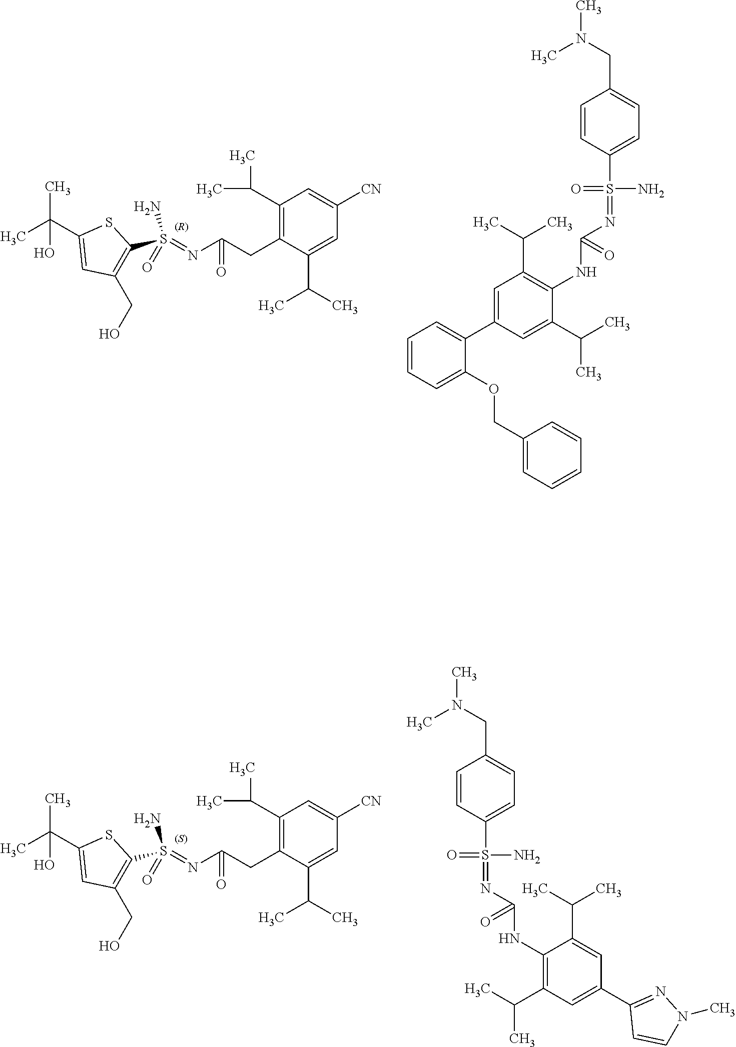

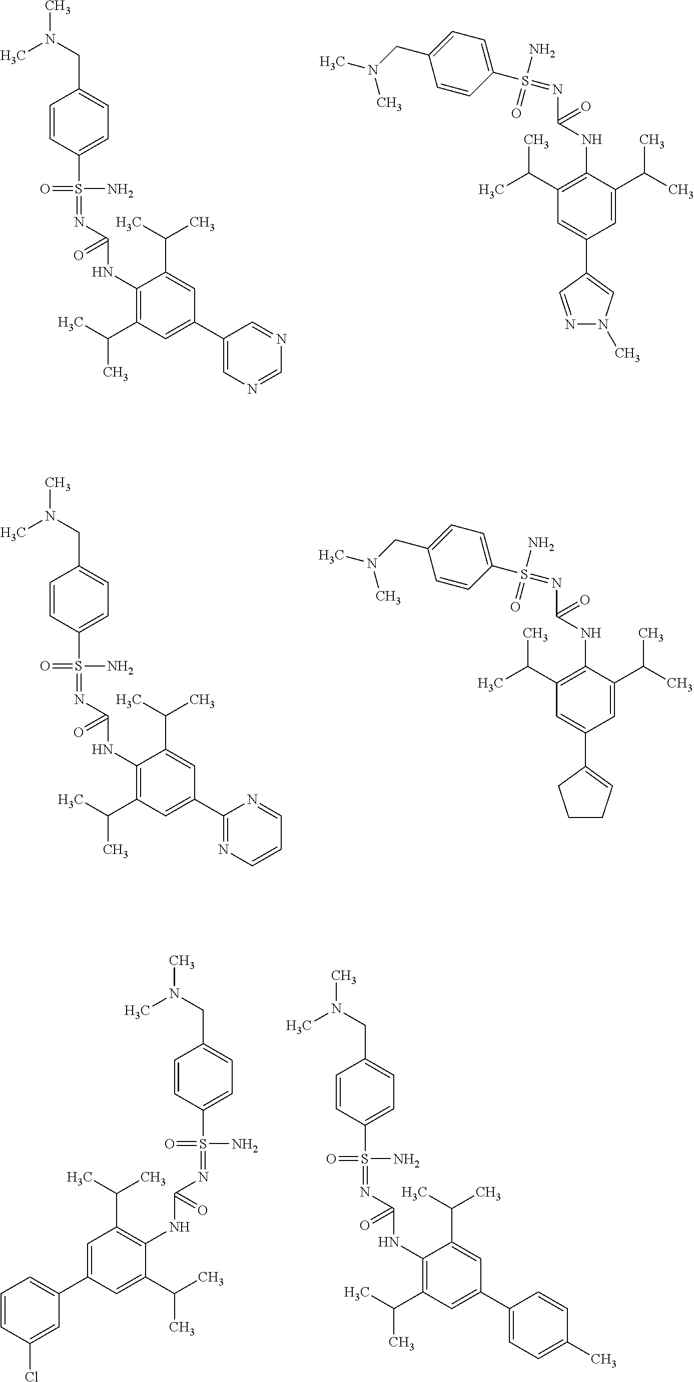

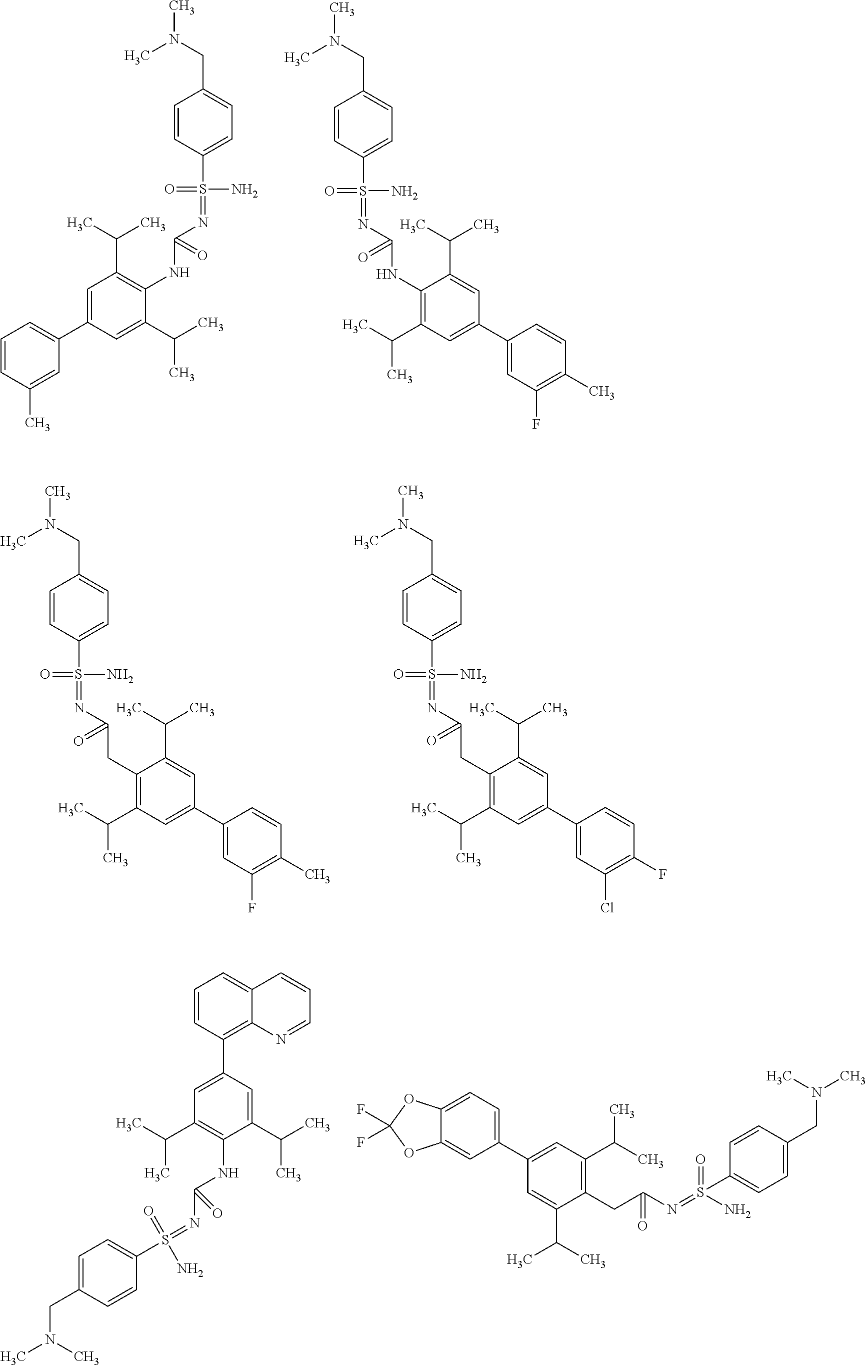

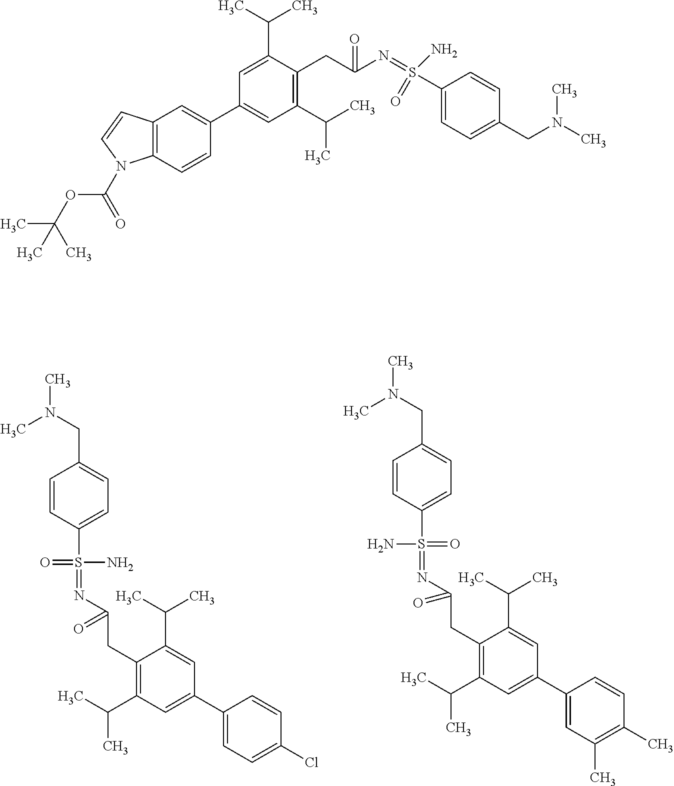

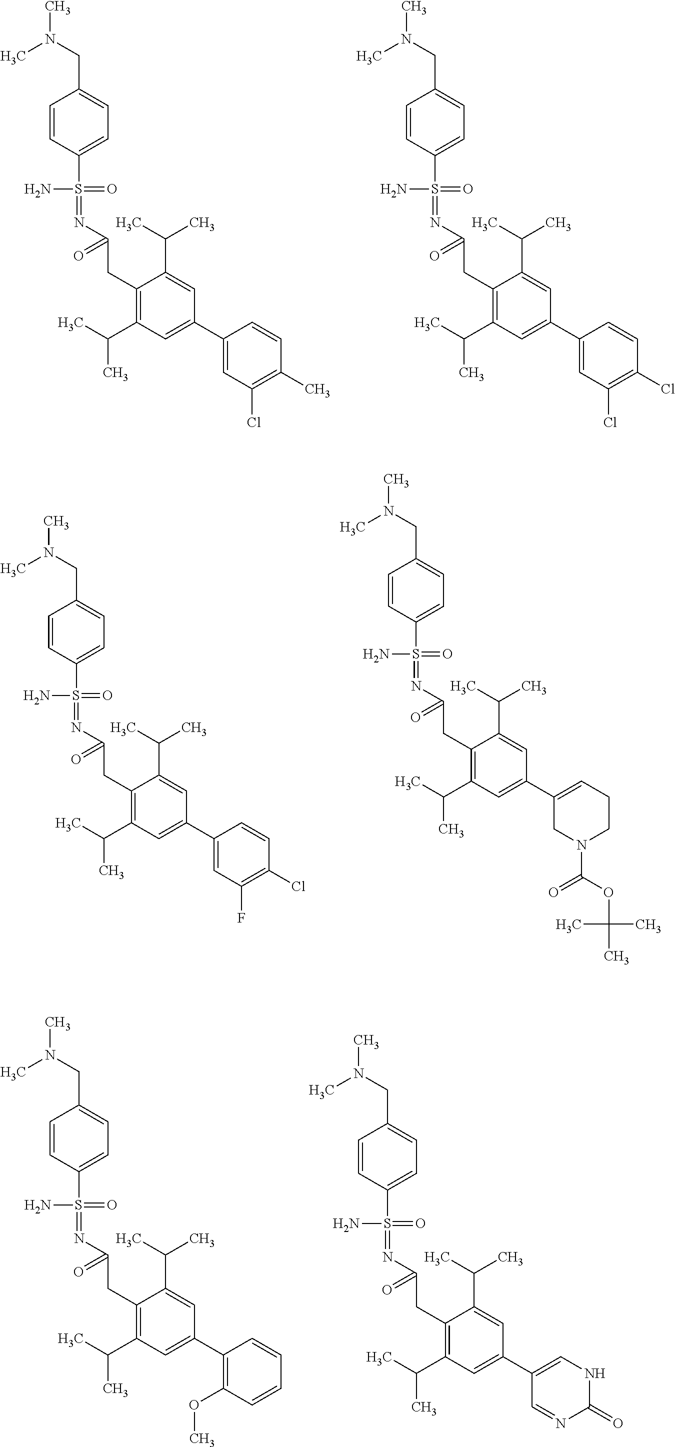

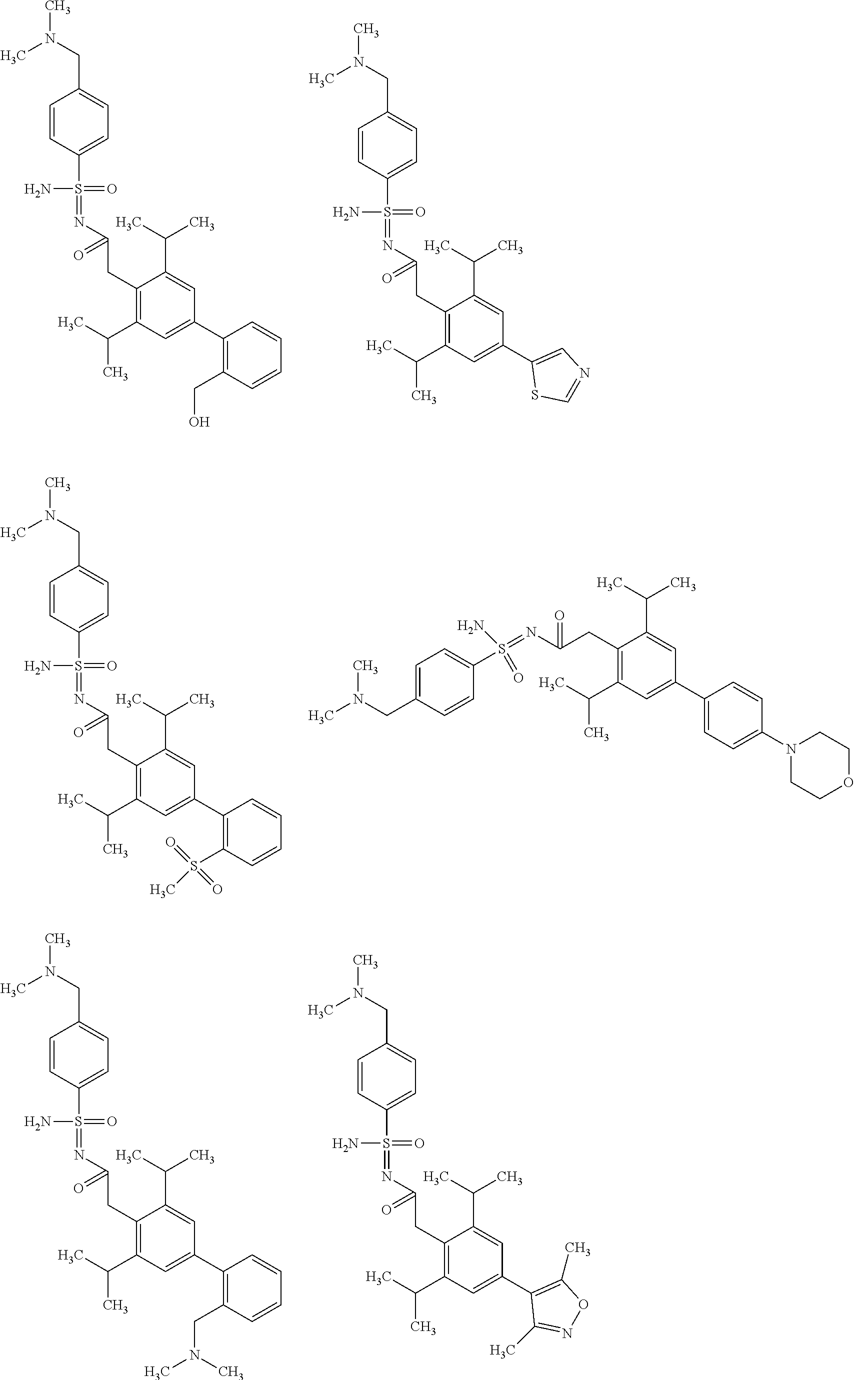 [SMILES: CC(C)c1cc(-c2ccccc2CO)cc(C(C)C)c1CC(=O)N=S(N)(=O)c1ccc(CN(C)C)cc1 ; CC(C)c1cc(-c2cncs2)cc(C(C)C)c1CC(=O)N=S(N)(=O)c1ccc(CN(C)C)cc1 ; CC(C)c1cc(-c2ccccc2S(C)(=O)=O)cc(C(C)C)c1CC(=O)N=S(N)(=O)c1ccc(CN(C)C)cc1 ; CC(C)c1cc(-c2ccc(N3CCOCC3)cc2)cc(C(C)C)c1CC(=O)N=S(N)(=O)c1ccc(CN(C)C)cc1 ; CC(C)c1cc(-c2ccccc2CN(C)C)cc(C(C)C)c1CC(=O)N=S(N)(=O)c1ccc(CN(C)C)cc1 ; Cc1noc(C)c1-c1cc(C(C)C)c(CC(=O)N=S(N)(=O)c2ccc(CN(C)C)cc2)c(C(C)C)c1]

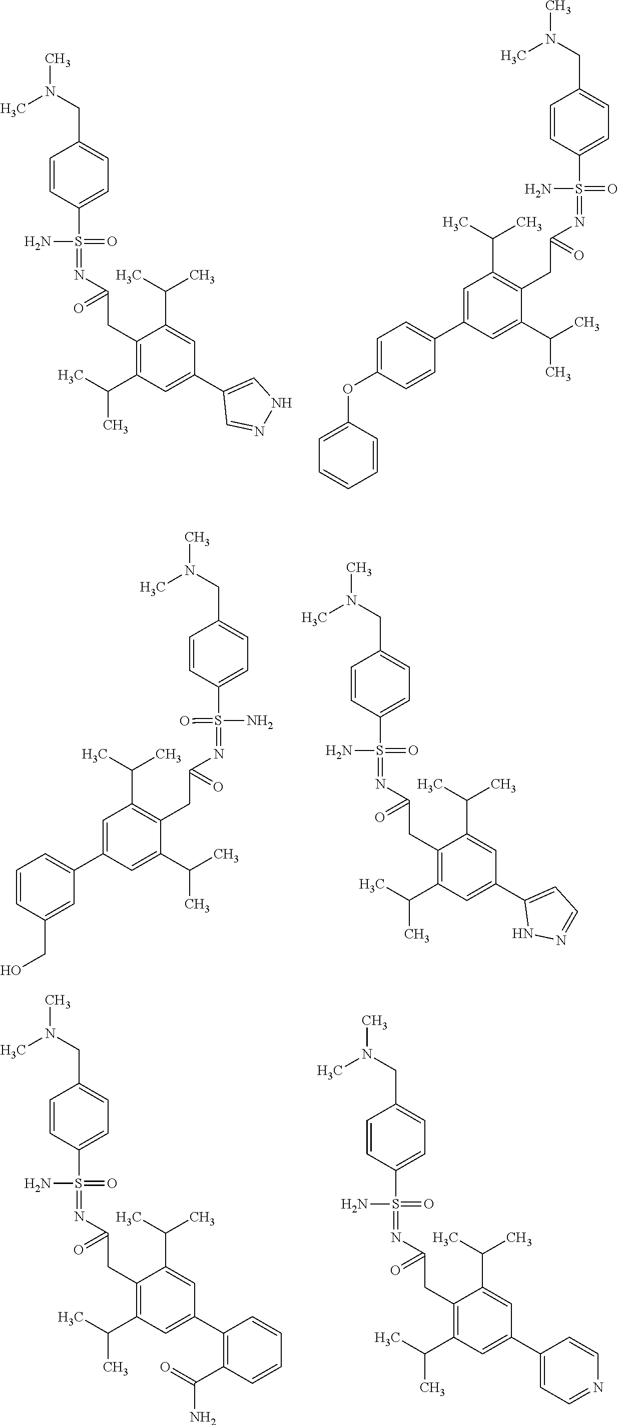

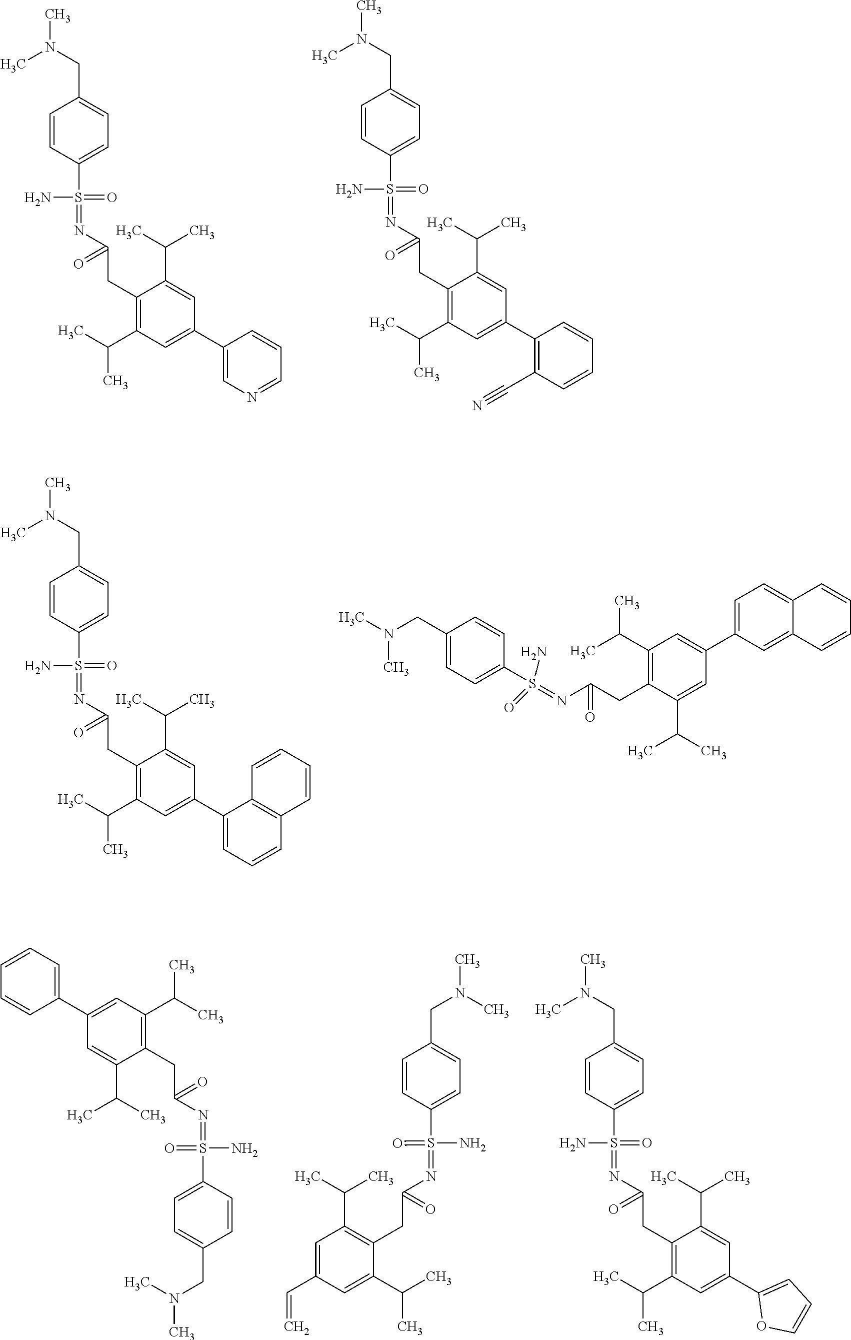

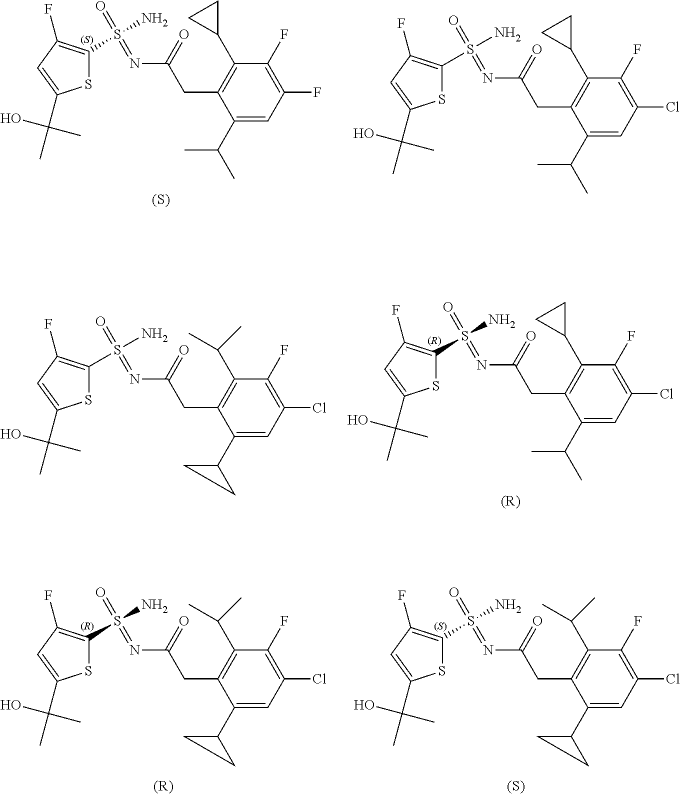

or a pharmaceutically acceptable salt thereof.

In any of the embodiments described herein, the compound of Formula AA is not one of the following:

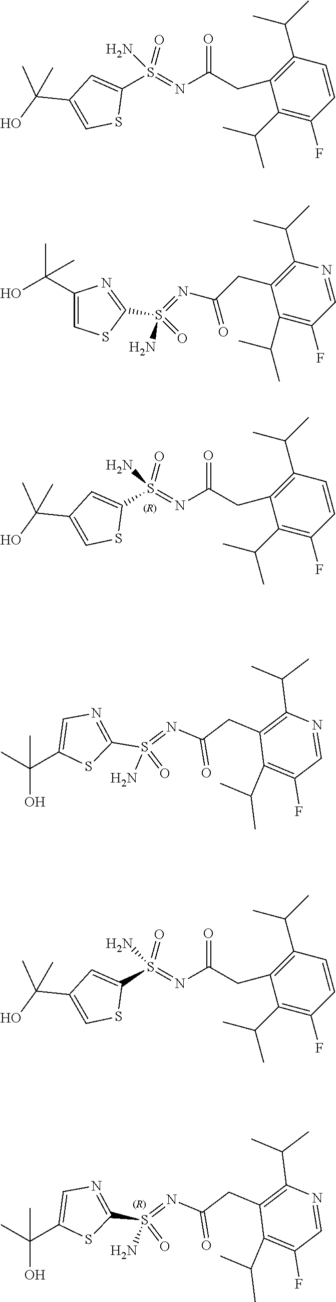

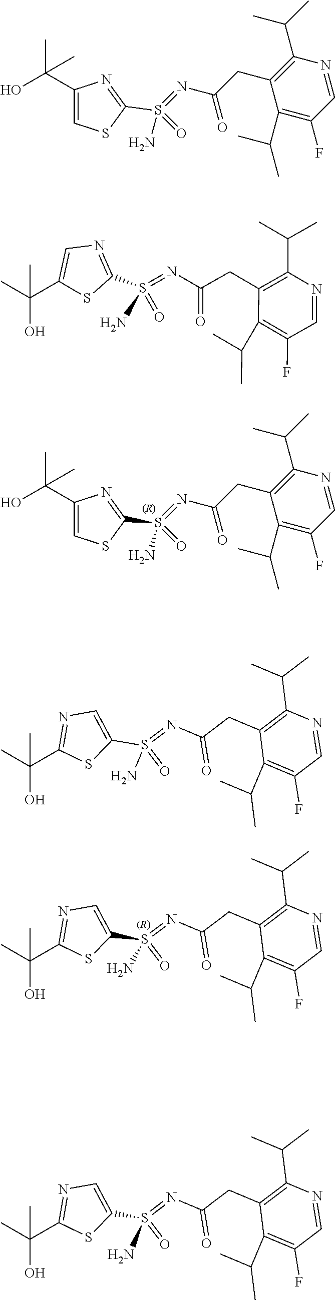

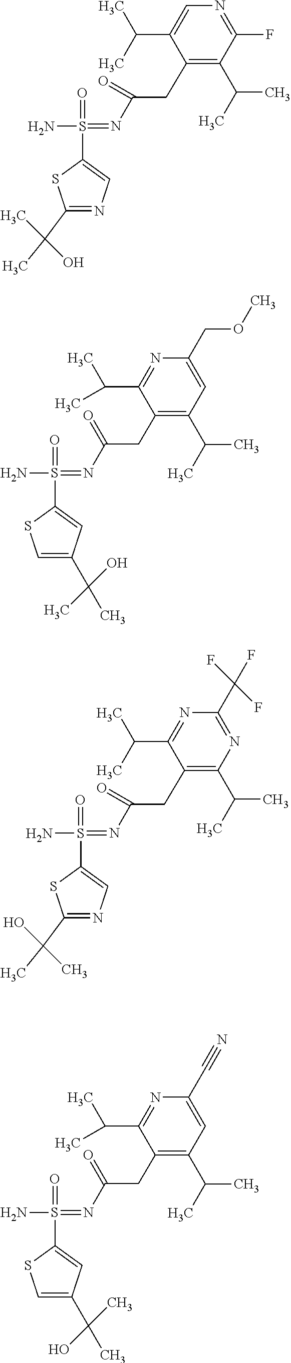 [SMILES: CC(C)c1cnc(F)c(C(C)C)c1CC(=O)N=S(N)(=O)c1cnc(C(C)(C)O)s1 ; COCc1cc(C(C)C)c(CC(=O)N=S(N)(=O)c2cc(C(C)(C)O)cs2)c(C(C)C)n1 ; CC(C)c1nc(C(F)(F)F)nc(C(C)C)c1CC(=O)N=S(N)(=O)c1cnc(C(C)(C)O)s1 ; CC(C)c1cc(C#N)nc(C(C)C)c1CC(=O)N=S(N)(=O)c1cc(C(C)(C)O)cs1]

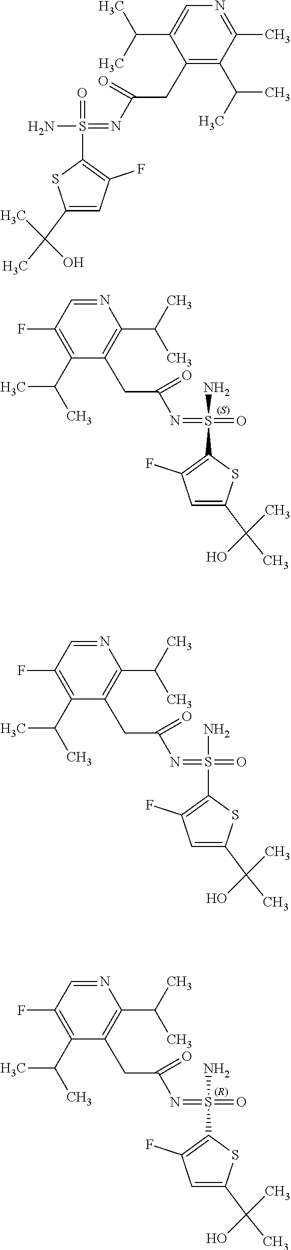

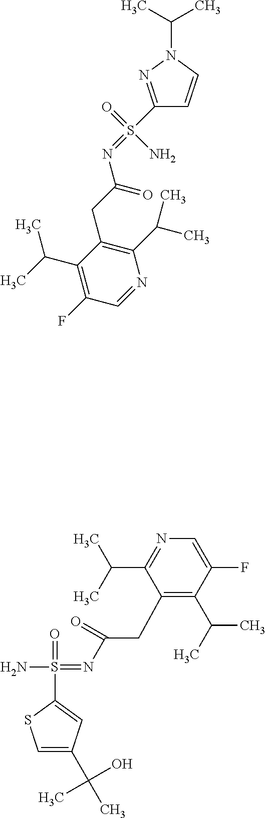 [SMILES: CC(C)c1ncc(F)c(C(C)C)c1CC(=O)N=S(N)(=O)c1ccn(C(C)C)n1 ; CC(C)c1ncc(F)c(C(C)C)c1CC(=O)N=S(N)(=O)c1cc(C(C)(C)O)cs1]

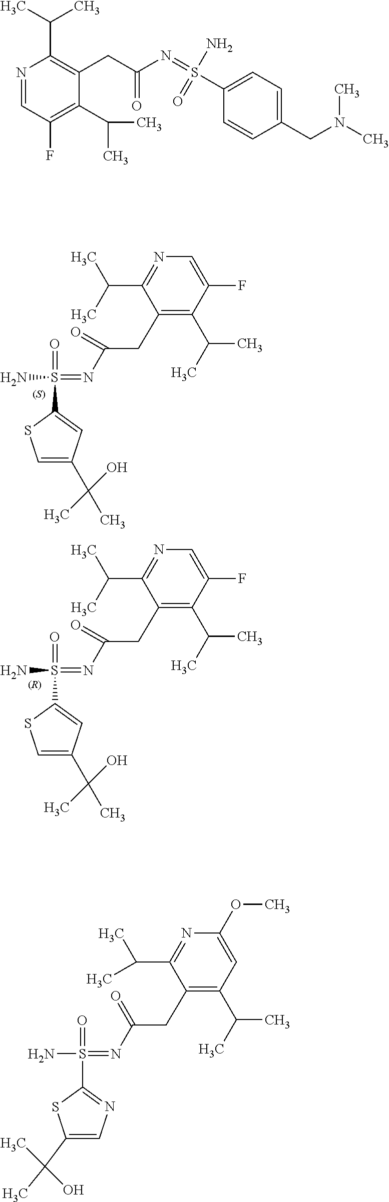

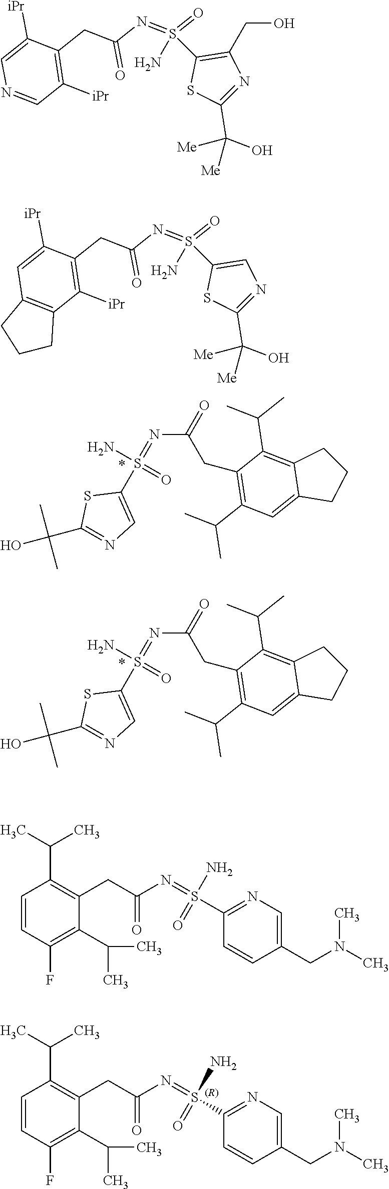

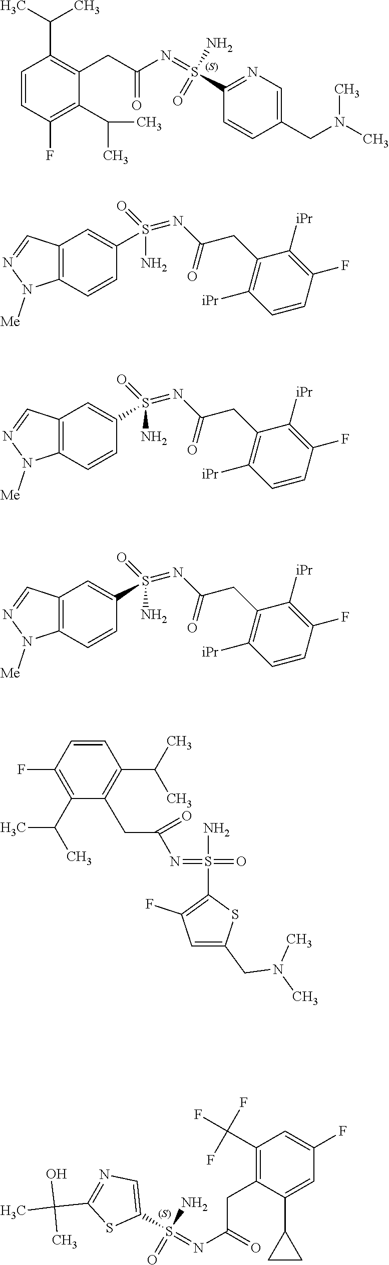

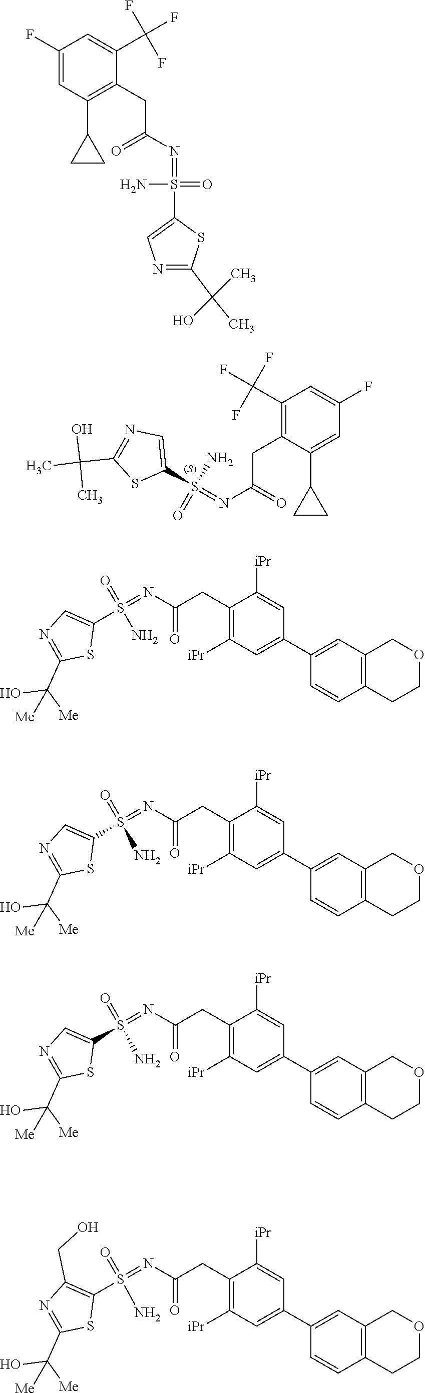

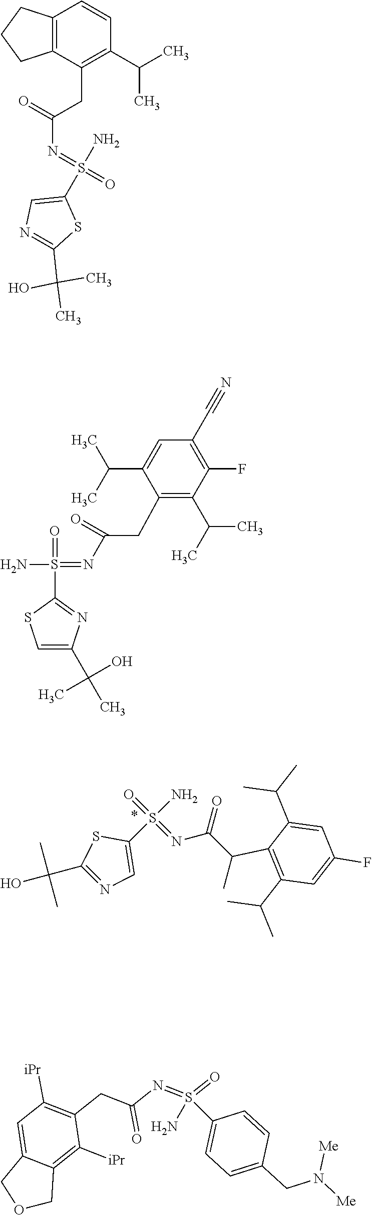 [SMILES: CC(C)c1ccc2c(c1CC(=O)N=S(N)(=O)c1cnc(C(C)(C)O)s1)CCC2 ; CC(C)c1cc(C#N)c(F)c(C(C)C)c1CC(=O)N=S(N)(=O)c1nc(C(C)(C)O)cs1 ; CC(C)c1cc(F)cc(C(C)C)c1C(C)C(=O)N=S(N)(=O)c1cnc(C(C)(C)O)s1 ; CC(C)c1cc2c(c(C(C)C)c1CC(=O)N=S(N)(=O)c1ccc(CN(C)C)cc1)COC2]

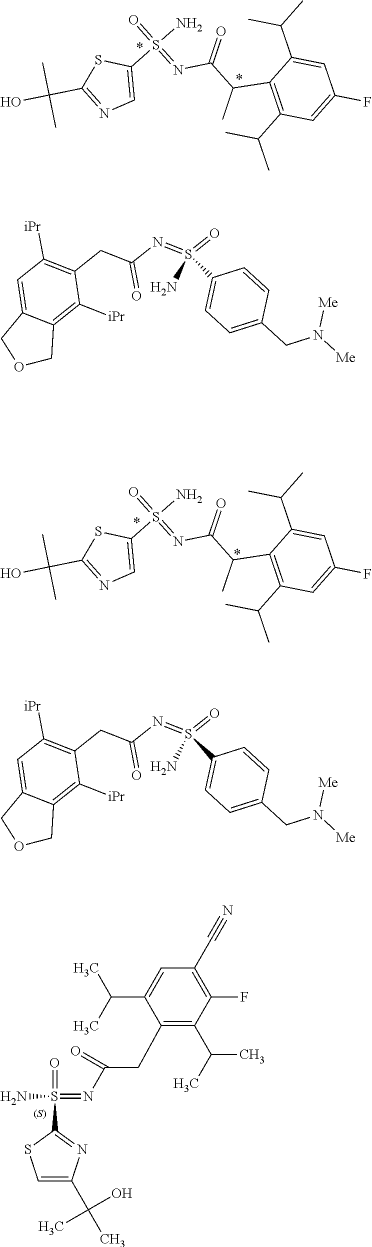

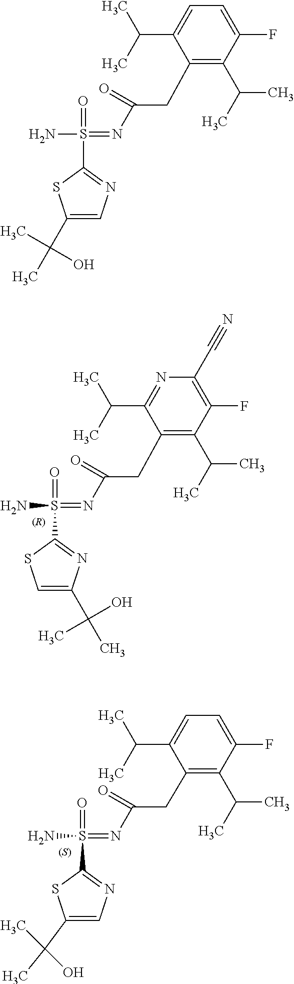

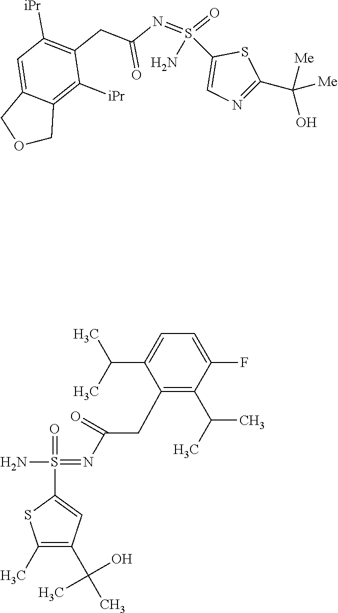 [SMILES: CC(C)c1cc2c(c(C(C)C)c1CC(=O)N=S(N)(=O)c1cnc(C(C)(C)O)s1)COC2 ; Cc1sc(S(N)(=O)=NC(=O)Cc2c(C(C)C)ccc(F)c2C(C)C)cc1C(C)(C)O]

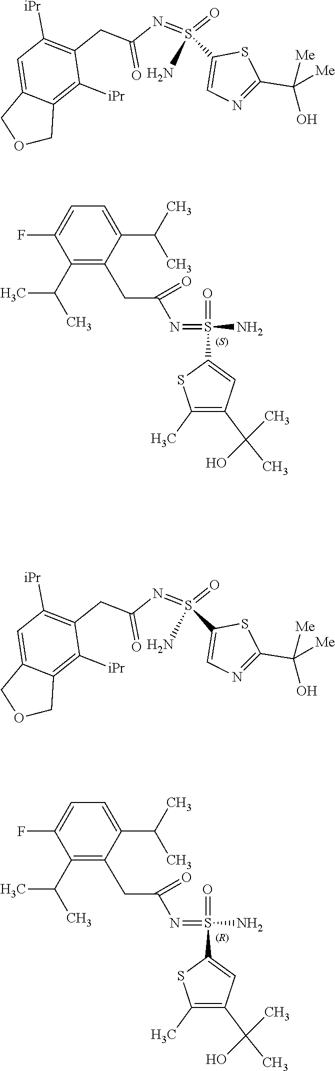

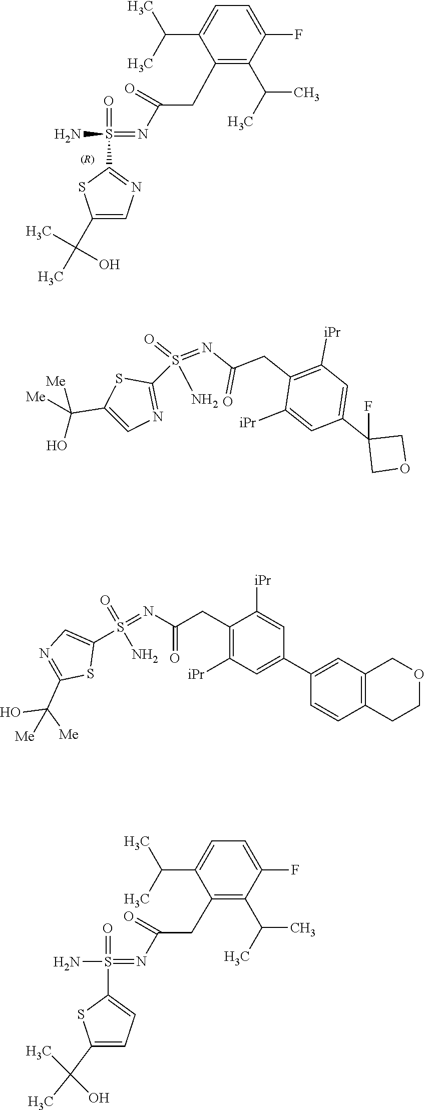

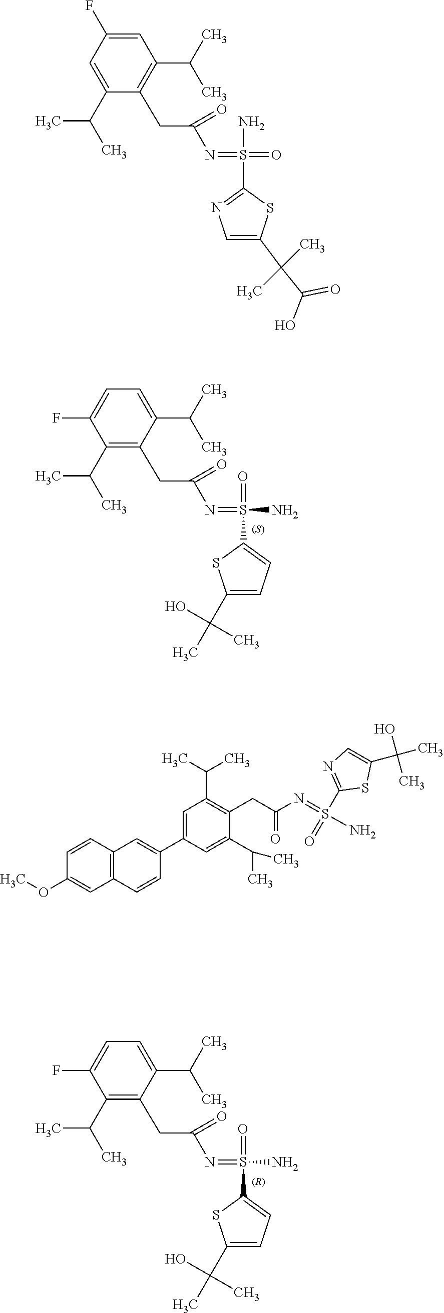

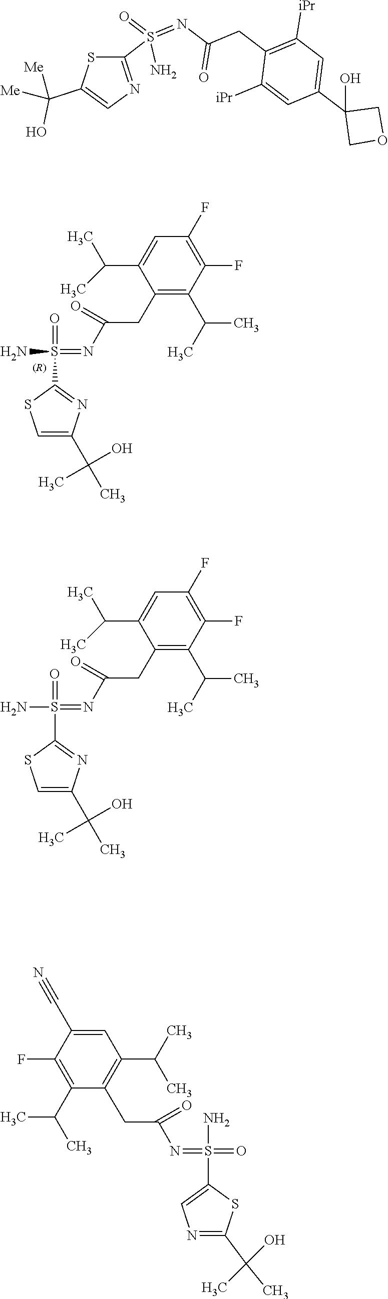

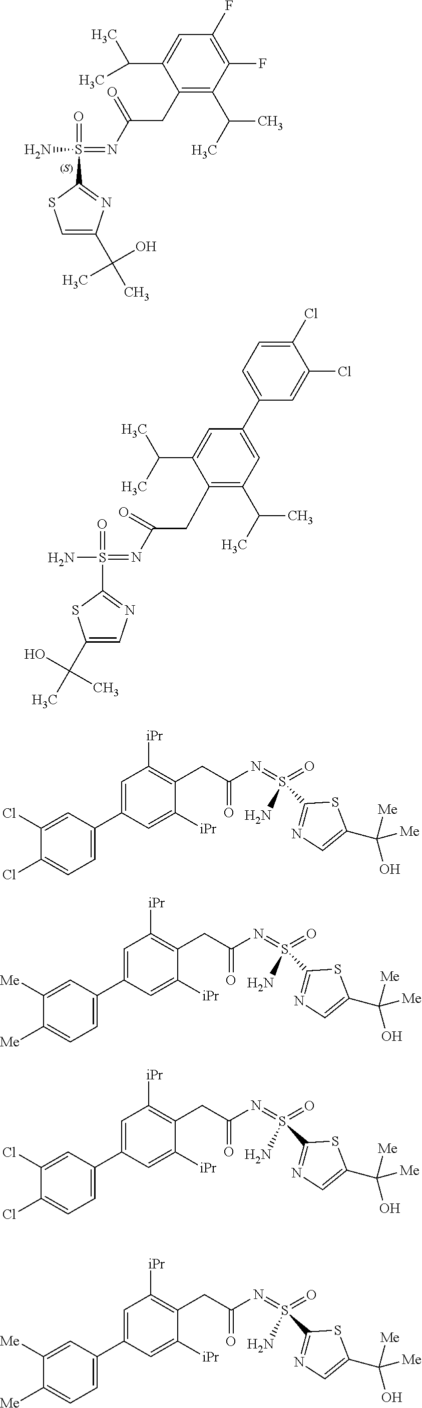

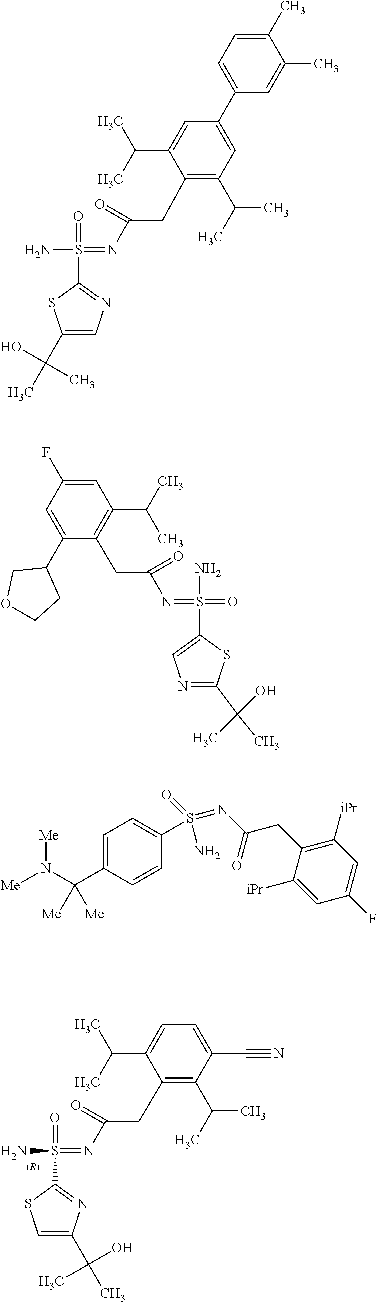

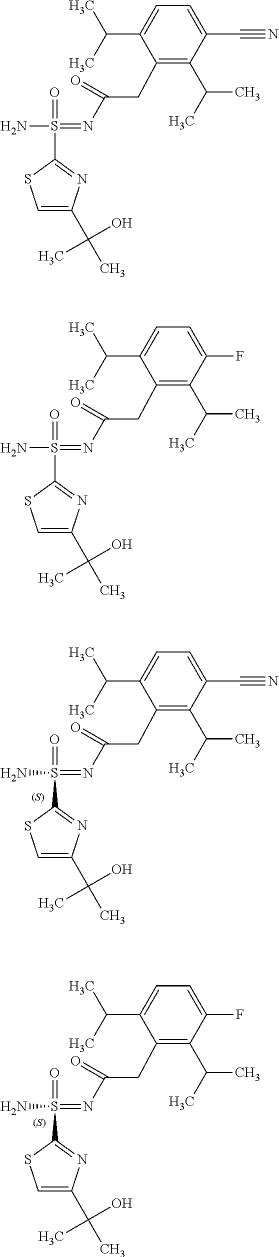

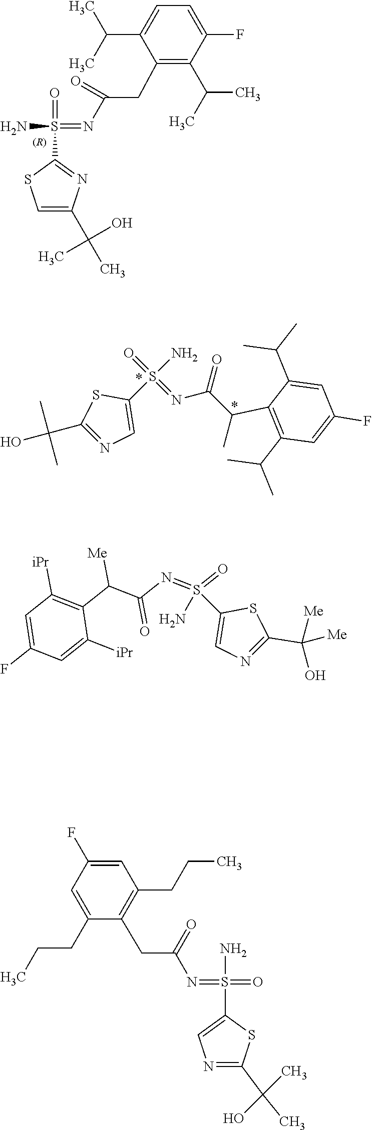

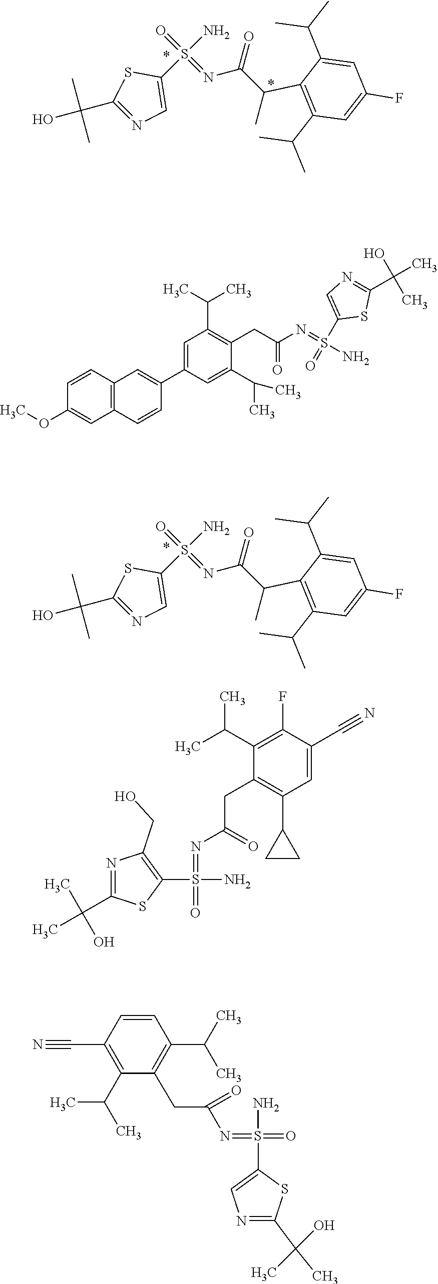

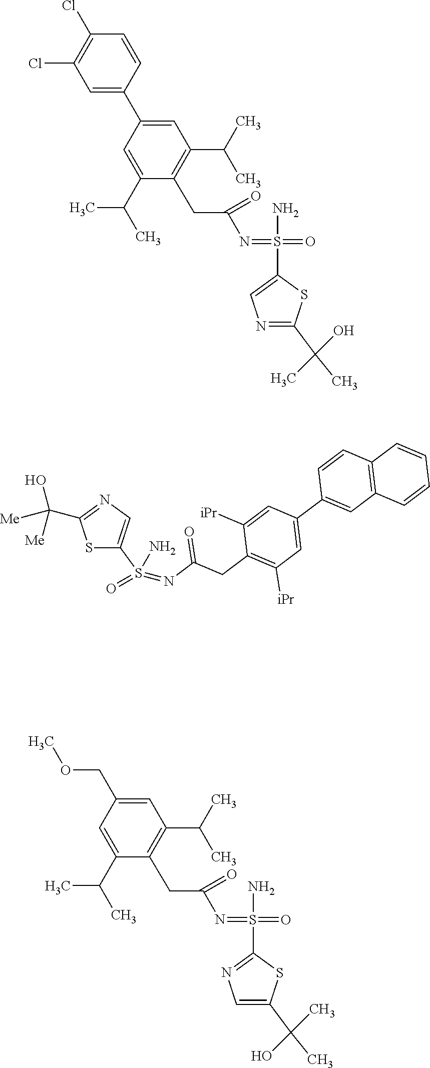 [SMILES: CC(C)c1cc(-c2ccc(Cl)c(Cl)c2)cc(C(C)C)c1CC(=O)N=S(N)(=O)c1cnc(C(C)(C)O)s1 ; CC(C)c1cc(-c2ccc3ccccc3c2)cc(C(C)C)c1CC(=O)N=S(N)(=O)c1cnc(C(C)(C)O)s1 ; COCc1cc(C(C)C)c(CC(=O)N=S(N)(=O)c2ncc(C(C)(C)O)s2)c(C(C)C)c1]

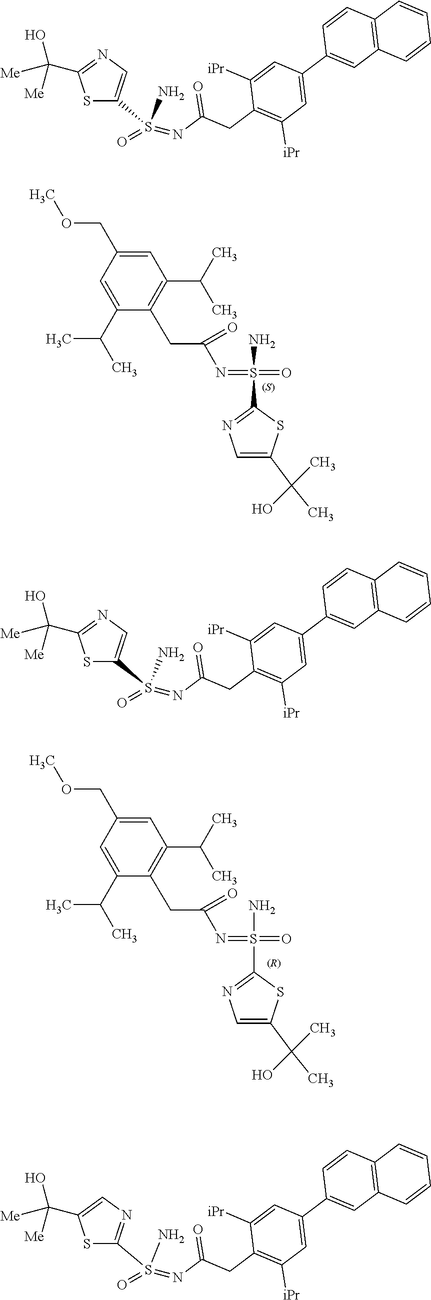

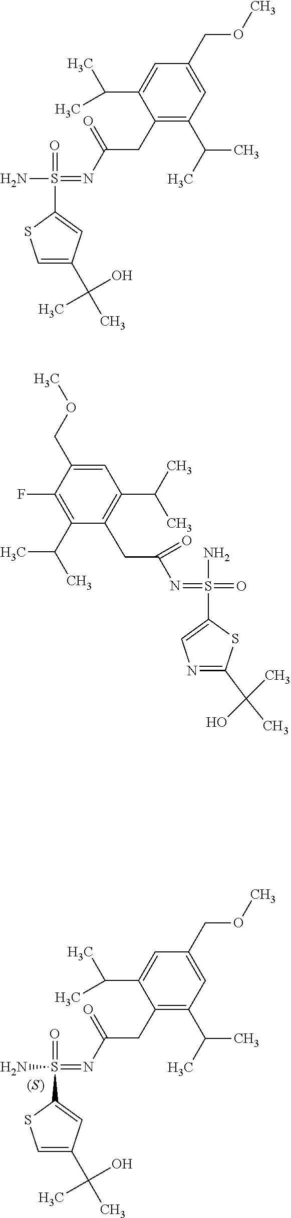

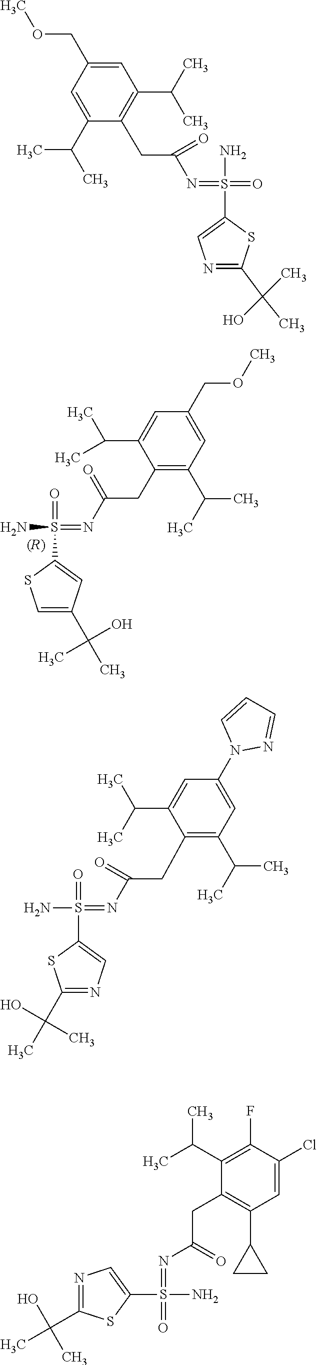

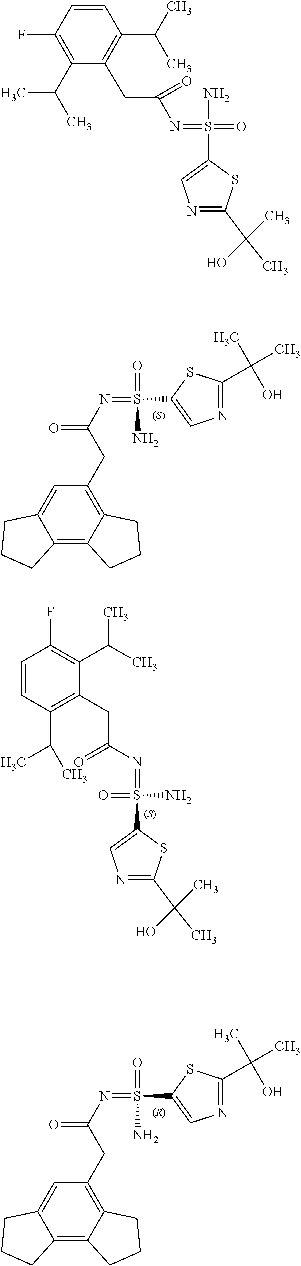

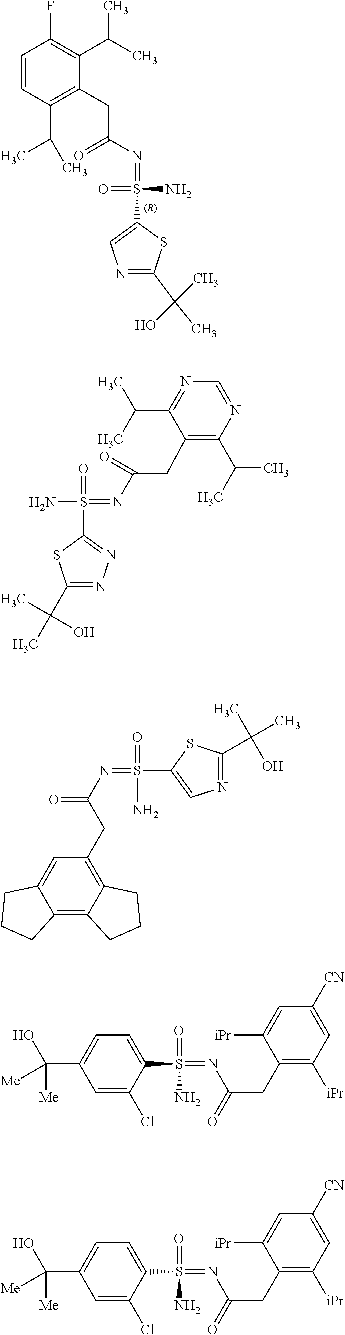

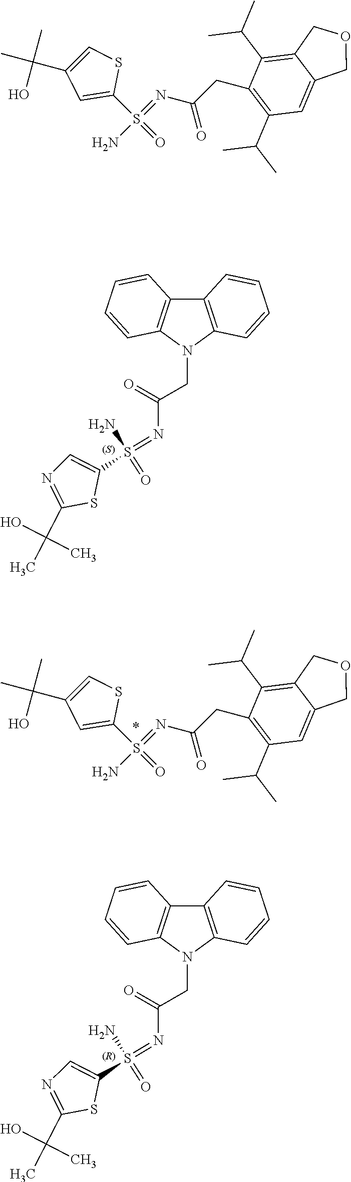

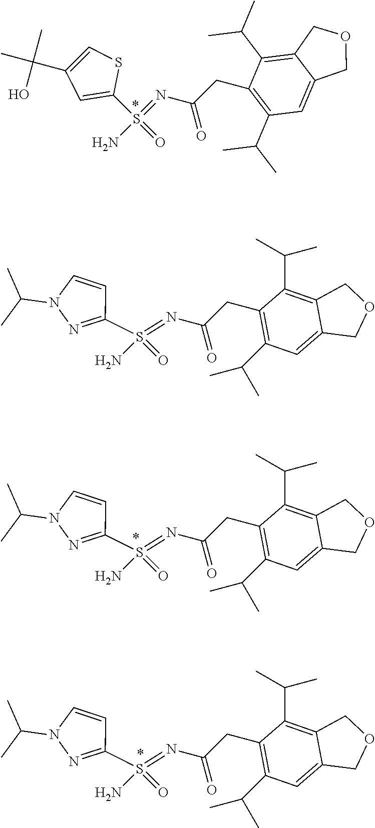 [SMILES: CC(C)c1cc2c(c(C(C)C)c1CC(=O)N=S(N)(=O)c1cc(C(C)(C)O)cs1)COC2 ; CC(C)c1cc2c(c(C(C)C)c1CC(=O)N=S(N)(=O)c1ccn(C(C)C)n1)COC2 ; CC(C)c1cc2c(c(C(C)C)c1CC(=O)N=S(N)(=O)c1ccn(C(C)C)n1)COC2 ; CC(C)c1cc2c(c(C(C)C)c1CC(=O)N=S(N)(=O)c1ccn(C(C)C)n1)COC2]

or a pharmaceutically acceptable salt thereof.

In any of the embodiments described herein, the compound of Formula AA is not one of the following:

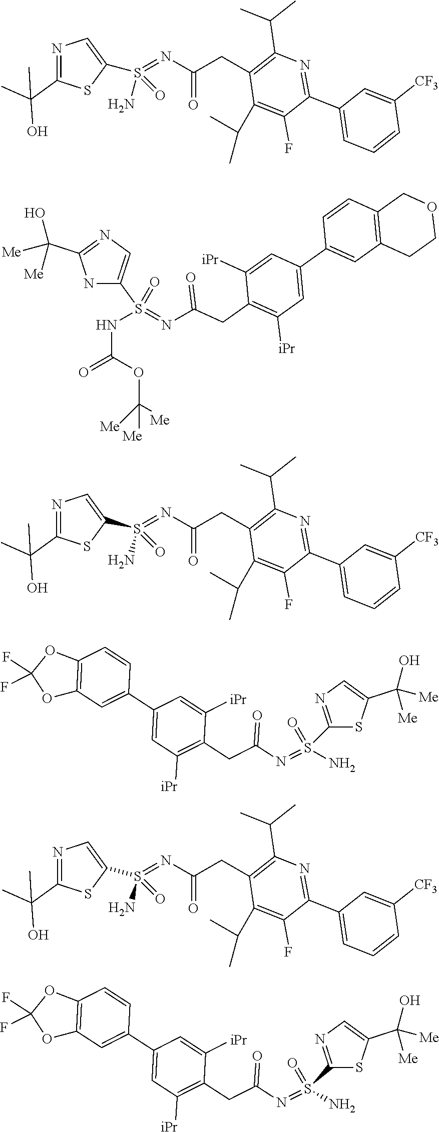

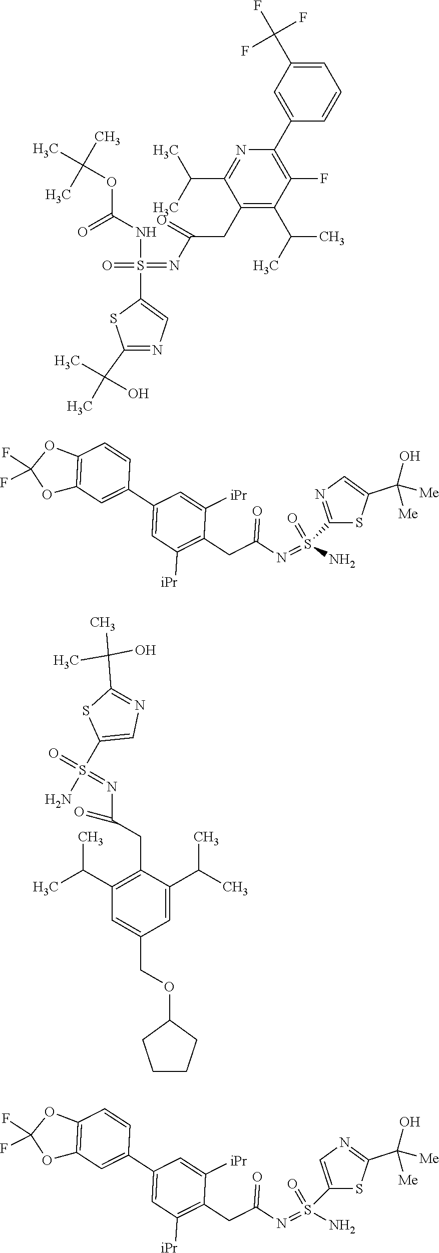

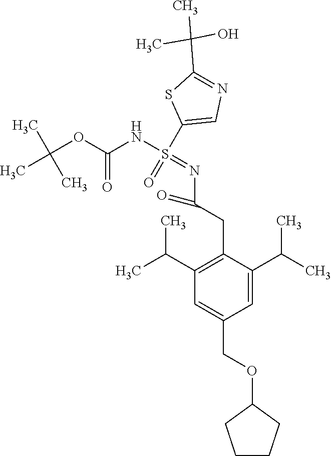 [SMILES: CC(C)c1cc(COC2CCCC2)cc(C(C)C)c1CC(=O)N=S(=O)(NC(=O)OC(C)(C)C)c1cnc(C(C)(C)O)s1]

or a pharmaceutically acceptable salt thereof.

In some embodiments the variables shown in the formulae herein are as follows:

### The Variables m and n

In some embodiments m=0, 1, or 2.

In some embodiments m=0 or 1.

In some embodiments m=1 or 2.

In some embodiments m=0 or 2.

In some embodiments m=0.

In some embodiments m=1.

In some embodiments m=2.

In some embodiments n=0, 1, or 2.

In some embodiments n=0 or 1.

In some embodiments n=1 or 2.

In some embodiments n=0 or 2.

In some embodiments n=0.

In some embodiments n=1.

In some embodiments n=2.

In some embodiments, m=0 and n=0.

In some embodiments, m=1 and n=0.

In some embodiments, m=1 and n=1.

### The Ring A and Substitutions on the Ring A

In some embodiments, A is a 5-10-membered (e.g., 5-6-membered) monocyclic or bicyclic heteroaryl or a C₆-C₁₀ monocyclic or bicyclic aryl, such as phenyl.

In some embodiments, A is a 5-10-membered (e.g., 5-6-membered) monocyclic or bicyclic heteroaryl.

In some embodiments, A is a 5-membered heteroaryl containing a sulfur and optionally one or more nitrogens.

In some embodiments, A is a 6-membered heteroaryl.

In some embodiments, A is a C₆-C₁₀ (e.g., C₆) monocyclic or bicyclic aryl.

In some embodiments, A is phenyl optionally substituted with 1 or 2 R¹ and optionally substituted with 1 or 2 R².

In some embodiments, A is furanyl optionally substituted with 1 or 2 R¹ and optionally substituted with 1 or 2 R².

In some embodiments, A is thiophenyl optionally substituted with 1 or 2 R¹ and optionally substituted with 1 or 2 R².

In some embodiments, A is oxazolyl optionally substituted with 1 or 2 R¹ and optionally substituted with 1 or 2 R².

In some embodiments, A is thiazolyl optionally substituted with 1 or 2 R¹ and optionally substituted with 1 or 2 R².

In some embodiments, A is pyrazolyl optionally substituted with 1 or 2 R¹ and optionally substituted with 1 or 2 R².

In some embodiments, A is imidazolyl optionally substituted with 1 or 2 R¹ and optionally substituted with 1 or 2 R².

In some embodiments, A is pyrrolyl optionally substituted with 1 or 2 R¹ and optionally substituted with 1 or 2 R².

In some embodiments, A is oxazolyl optionally substituted with 1 or 2 R¹ and optionally substituted with 1 or 2 R².

In some embodiments, A is furanyl optionally substituted with 1 or 2 R¹ and optionally substituted with 1 or 2 R².

In some embodiments, A is isoxazolyl optionally substituted with 1 or 2 R¹ and optionally substituted with 1 or 2 R².

In some embodiments, A is isothiazolyl optionally substituted with 1 or 2 R¹ and optionally substituted with 1 or 2 R².

In some embodiments, A is triazolyl (e.g., 1,2,3-triazolyl or 1,2,4-triazolyl) optionally substituted with 1 R¹ and optionally substituted with 1 R².

In some embodiments, A is pyridyl optionally substituted with 1 or 2 R¹ and optionally substituted with 1 or 2 R².

In some embodiments, A is pyridimidinyl optionally substituted with 1 or 2 R¹ and optionally substituted with 1 or 2 R².

In some embodiments, A is pyrazinyl optionally substituted with 1 or 2 R¹ and optionally substituted with 1 or 2 R².

In some embodiments, A is pyridazinyl optionally substituted with 1 or 2 R¹ and optionally substituted with 1 or 2 R².

In some embodiments, A is triazinyl optionally substituted with 1 or 2 R¹ and optionally substituted with 1 or 2 R².

In some embodiments, A is indazolyl optionally substituted with 1 or 2 R¹ and optionally substituted with 1 or 2 R².

In some embodiments, A is phenyl substituted with 1 R¹ and optionally substituted with 1 R².

In some embodiments, A is furanyl substituted with 1 R¹ and optionally substituted with 1 R².

In some embodiments, A is thiophenyl substituted with 1 R¹ and optionally substituted with 1 R².

In some embodiments, A is oxazolyl substituted with 1 R¹ and optionally substituted with 1 R².

In some embodiments, A is thiazolyl substituted with 1 R¹ and optionally substituted with 1 R².

In some embodiments, A is pyrazolyl substituted with 1 R¹ and optionally substituted with 1 R².

In some embodiments, A is imidazolyl substituted with 1 R¹ and optionally substituted with 1 R².

In some embodiments, A is pyrrolyl substituted with 1 R¹ and optionally substituted with 1 R².

In some embodiments, A is oxazolyl substituted with 1 R¹ and optionally substituted with 1 R².

In some embodiments, A is furanyl substituted with 1 R¹ and optionally substituted with 1 R².

In some embodiments, A is isoxazolyl substituted with 1 R¹ and optionally substituted with 1 R².

In some embodiments, A is isothiazolyl substituted with 1 R¹ and optionally substituted with 1 R².

In some embodiments, A is triazolyl (e.g., 1,2,3-triazolyl or 1,2,4-triazolyl) substituted with 1 R¹ and optionally substituted with 1 R².

In some embodiments, A is pyridyl substituted with 1 R¹ and optionally substituted with 1 R².

In some embodiments, A is pyridimidinyl substituted with 1 R¹ and optionally substituted with 1 R².

In some embodiments, A is pyrazinyl substituted with 1 R¹ and optionally substituted with 1 R².

In some embodiments, A is pyridazinyl substituted with 1 R¹ and optionally substituted with 1 R².

In some embodiments, A is triazinyl substituted with 1 R¹ and optionally substituted with 1 R².

In some embodiments, A is phenyl substituted with 1 R¹ and optionally substituted with 1 R².

In some embodiments, A is furanyl substituted with 1 R¹ and substituted with 1 R².

In some embodiments, A is thiophenyl substituted with 1 R¹ and substituted with 1 R².

In some embodiments, A is oxazolyl substituted with 1 R¹ and substituted with 1 R².

In some embodiments, A is thiazolyl substituted with 1 R¹ and substituted with 1 R².

In some embodiments, A is pyrazolyl substituted with 1 R¹ and substituted with 1 R².

In some embodiments, A is imidazolyl substituted with 1 R¹ and substituted with 1 R².

In some embodiments, A is pyrrolyl substituted with 1 R¹ and substituted with 1 R².

In some embodiments, A is oxazolyl substituted with 1 R¹ and substituted with 1 R².

In some embodiments, A is furanyl substituted with 1 R¹ and substituted with 1 R².

In some embodiments, A is isoxazolyl substituted with 1 R¹ and substituted with 1 R².

In some embodiments, A is isothiazolyl substituted with 1 R¹ and substituted with 1 R².

In some embodiments, A is triazolyl (e.g., 1,2,3-triazolyl or 1,2,4-triazolyl) substituted with 1 R¹ and substituted with 1 R².

In some embodiments, A is pyridyl substituted with 1 R¹ and substituted with 1 R².

In some embodiments, A is pyridimidinyl substituted with 1 R¹ and substituted with 1 R².

In some embodiments, A is pyrazinyl substituted with 1 R¹ and substituted with 1 R².

In some embodiments, A is pyridazinyl substituted with 1 R¹ and substituted with 1 R².

In some embodiments, A is triazinyl substituted with 1 R¹ and substituted with 1 R².

In some embodiments, A is phenyl, m is 0, 1, or 2; and n is 0, 1, or 2.

In some embodiments, A is furanyl, m is 0, 1, or 2, and n is 0, 1, or 2.

In some embodiments, A is thiophenyl, m is 0, 1, or 2, and n is 0, 1, or 2.

In some embodiments, A is oxazolyl, m is 0, 1, or 2, and n is 0, 1, or 2.

In some embodiments, A is thiazolyl, m is 0, 1, or 2, and n is 0, 1, or 2.

In some embodiments, A is pyrazolyl, m is 0, 1, or 2, and n is 0, 1, or 2.

In some embodiments, A is pyridyl m is 0, 1, or 2, and n is 0, 1, or 2.

In some embodiments, A is phenyl, m is 0 or 1, and n is 0 or 1.

In some embodiments, A is furanyl, m is 0 or 1, and n is 0 or 1.

In some embodiments, A is thiophenyl, m is 1 and n is 0 or 1.

In some embodiments, A is oxazolyl, m is 1 and n is 0 or 1.

In some embodiments, A is thiazolyl, m is 1 and n is 0 or 1.

In some embodiments, A is pyrazolyl, m is 1 and n is 0 or 1.

In some embodiments, A is pyridyl, m is 1 and n is 0 or 1.

In some embodiments, A is phenyl, m is 1 and n is 1.

In some embodiments, A is furanyl, m is 1 and n is 1.

In some embodiments, A is thiophenyl, m is 1 and n is 1.

In some embodiments, A is oxazolyl, m is 1 and n is 1.

In some embodiments, A is thiazolyl, m is 1 and n is 1.

In some embodiments, A is pyrazolyl, m is 1 and n is 1.

In some embodiments, A is pyridyl, m is 1 and n is 1.

In some embodiments, A is phenyl, m is 0 or 1, and n is 0, 1, or 2.

In some embodiments, A is furanyl, m is 0 or 1, and n is 0, 1, or 2.

In some embodiments, A is thiophenyl, m is 0 or 1, and n is 0, 1, or 2.

In some embodiments, A is oxazolyl, m is 0 or 1, and n is 0, 1, or 2.

In some embodiments, A is thiazolyl, m is 0 or 1, and n is 0, 1, or 2.

In some embodiments, A is pyrazolyl, m is 0 or 1, and n is 0, 1, or 2.

In some embodiments, A is pyridyl, m is 0 or 1, and n is 0, 1, or 2.

In some embodiments, A is phenyl, m is 0, and n is 0 or 1.

In some embodiments, A is furanyl, m is 0, and n is 0 or 1.

In some embodiments, A is thiophenyl, m is 0, and n is 0 or 1.

In some embodiments, A is oxazolyl, m is 0, and n is 0 or 1.

In some embodiments, A is thiazolyl, m is 0, and n is 0 or 1.

In some embodiments, A is pyrazolyl, m is 0, and n is 0 or 1.

In some embodiments, A is pyridyl, m is 0, and n is 0 or 1.

In some embodiments, A is thiazolyl, m is 1, and n is 1.

In some embodiments, A is pyrazolyl, m is 1 or 2, and n is 1 or 2.

In some embodiments, A is imidazolyl, m is 1 or 2, and n is 1 or 2.

In some embodiments, A is pyrrolyl, m is 1 or 2, and n is 1 or 2.

In some embodiments, A is oxazolyl, m is 1, and n is 1.

In some embodiments, A is furanyl, m is 1 or 2, and n is 1 or 2.

In some embodiments, A is isoxazolyl, m is 1, and n is 1.

In some embodiments, A is isothiazolyl, m is 1, and n is 1.

In some embodiments, A is triazolyl (e.g., 1,2,3-triazolyl or 1,2,4-triazolyl), m is 1, and n is 1.

In some embodiments, A is pyridinyl, m is 1 or 2, and n is 1 or 2.

In some embodiments, A is pyridimidinyl, m is 1 or 2, and n is 1 or 2.

In some embodiments, A is pyrazinyl, m is 1 or 2, and n is 1 or 2.

In some embodiments, A is pyridazinyl, m is 1 or 2, and n is 1 or 2.

In some embodiments, A is triazinyl, m is 1, and n is 1.

In some embodiments, A is one of the rings disclosed hereinbelow optionally substituted as disclosed hereinbelow, wherein in each case the bond that is shown as being broken by the wavy line [symbol] connects A to the S(O)(NHR³)═N moiety of Formula AA.

In some embodiments, the optionally substituted ring A is

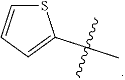 [SMILES: *c1cccs1]

In some embodiments, the optionally substituted ring A is

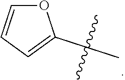 [SMILES: *c1ccco1]

In some embodiments, the optionally substituted ring A is

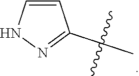 [SMILES: *c1cc[nH]n1]

In some embodiments, the optionally substituted ring A is

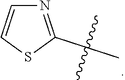 [SMILES: *c1nccs1]

In some embodiments, the optionally substituted ring A is

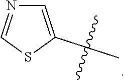 [SMILES: *c1cncs1]

In some embodiments, the optionally substituted ring A is

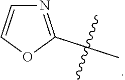 [SMILES: *c1ncco1]

In some embodiments, the optionally substituted ring A is

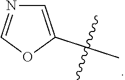 [SMILES: *c1cnco1]

In some embodiments, the optionally substituted ring A is

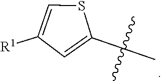

In some embodiments, the optionally substituted ring A is

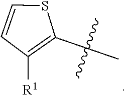

In some embodiments, the optionally substituted ring A is

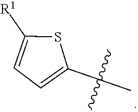

In some embodiments, the optionally substituted ring A is

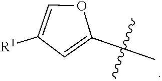

In some embodiments, the optionally substituted ring A is

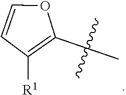

In some embodiments, the optionally substituted ring A is

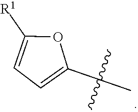

In some embodiments, the optionally substituted ring A is

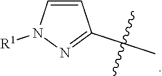

In some embodiments, the optionally substituted ring A is

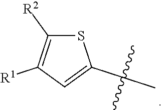

In some embodiments, the optionally substituted ring A is

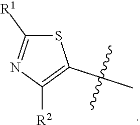

In some embodiments, the optionally substituted ring A is

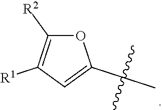

In some embodiments, the optionally substituted ring A is

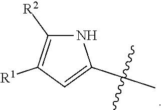

In some embodiments, the optionally substituted ring A is

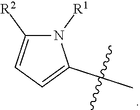

In some embodiments, the optionally substituted ring A is

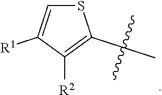

In some embodiments, the optionally substituted ring A is

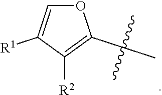

In some embodiments, the optionally substituted ring A is

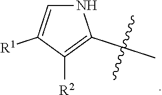

In some embodiments, the optionally substituted ring A is

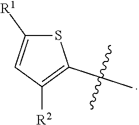

In some embodiments, the optionally substituted ring A is

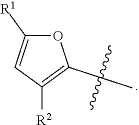

In some embodiments, the substituted ring A is

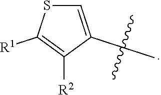

In some embodiments, the substituted ring A is

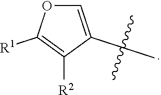

In some embodiments, the substituted ring A is

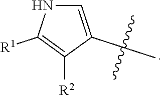

In some embodiments, the substituted ring A is

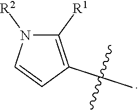

In some embodiments, the substituted ring A is

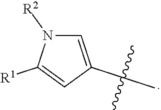

In some embodiments, the optionally substituted ring A is

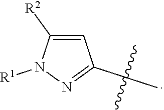

In some embodiments, the optionally substituted ring A is

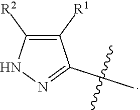

In some embodiments, the optionally substituted ring A is

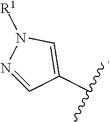

In some embodiments, the optionally substituted ring A is

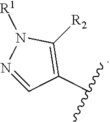

In some embodiments, the optionally substituted ring A is

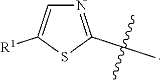

In some embodiments, the optionally substituted ring A is

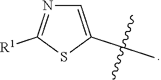

In some embodiments, the optionally substituted ring A is

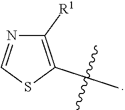

In some embodiments, the optionally substituted ring A is

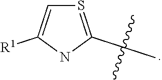

In some embodiments, the optionally substituted ring A is

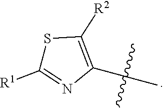

In some embodiments, the optionally substituted ring A is

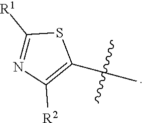

In some embodiments, the optionally substituted ring A is

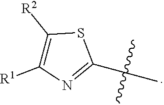

In some embodiments, the optionally substituted ring A is

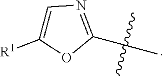

In some embodiments, the optionally substituted ring A is

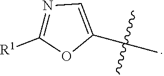

In some embodiments, the optionally substituted ring A is

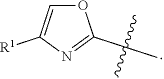

In some embodiments, the optionally substituted ring A is

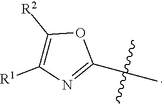

In some embodiments, the optionally substituted ring A is

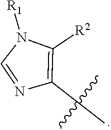

In some embodiments, the optionally substituted ring A is

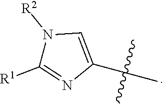

In some embodiments, the optionally substituted ring A is

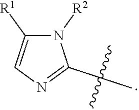

In some embodiments, the optionally substituted ring A is

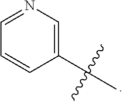 [SMILES: *c1cccnc1]

In some embodiments, the optionally substituted ring A is

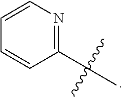 [SMILES: *c1ccccn1]

In some embodiments, the optionally substituted ring A is

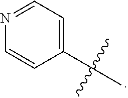 [SMILES: *c1ccncc1]

In some embodiments, the optionally substituted ring A is

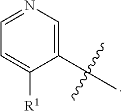

In some embodiments, the optionally substituted ring A is

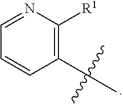

In some embodiments, the optionally substituted ring A is

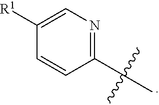

In some embodiments, the optionally substituted ring A is

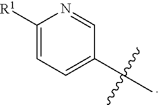

In some embodiments, the optionally substituted ring A is

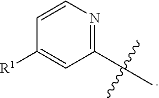

In some embodiments, the optionally substituted ring A is

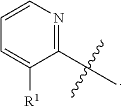

In some embodiments, the optionally substituted ring A is

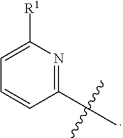

In some embodiments, the optionally substituted ring A is

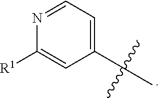

In some embodiments, the optionally substituted ring A is

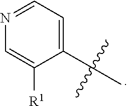

In some embodiments, the optionally substituted ring A is

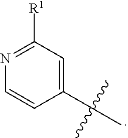

In some embodiments, the optionally substituted ring A is

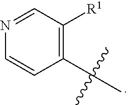

In some embodiments, the optionally substituted ring A is

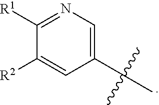

In some embodiments, the optionally substituted ring A is

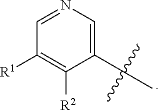

In some embodiments, the optionally substituted ring A is

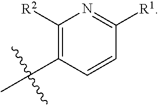

In some embodiments, the optionally substituted ring A is

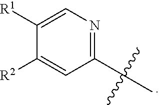

In some embodiments, the optionally substituted ring A is

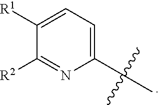

In some embodiments, the optionally substituted ring A is

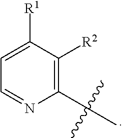

In some embodiments, the optionally substituted ring A is

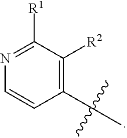

In some embodiments, the optionally substituted ring A is

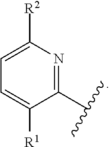

In some embodiments, the optionally substituted ring A is

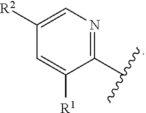

In some embodiments, the optionally substituted ring A is

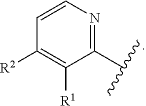

In some embodiments, the optionally substituted ring A is

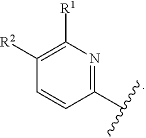

In some embodiments, the optionally substituted ring A is

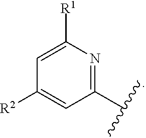

In some embodiments, the optionally substituted ring A is

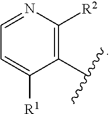

In some embodiments, the optionally substituted ring A is

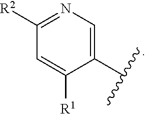

In some embodiments, the optionally substituted ring A is

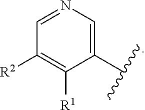

In some embodiments, the optionally substituted ring A is

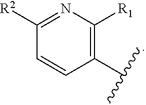

In some embodiments, the optionally substituted ring A is

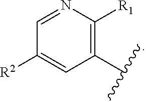

In some embodiments, the optionally substituted ring A is

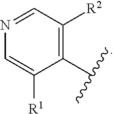

In some embodiments, the optionally substituted ring A is

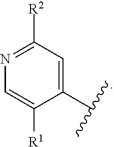

In some embodiments, the optionally substituted ring A is

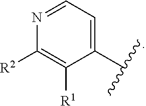

In some embodiments, the optionally substituted ring A is

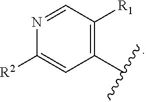

In some embodiments, the optionally substituted ring A is

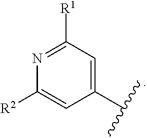

In some embodiments, the optionally substituted ring A is

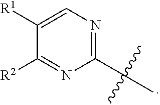

In some embodiments, the optionally substituted ring A is

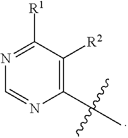

In some embodiments, the optionally substituted ring A is

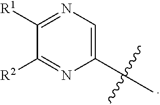

In some embodiments, the optionally substituted ring A is

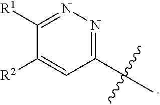

In some embodiments, the optionally substituted ring A is

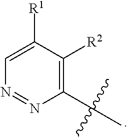

In some embodiments, the optionally substituted ring A is

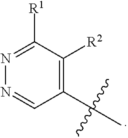

In some embodiments, the optionally substituted ring A is

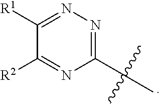

In some embodiments, the optionally substituted ring A is

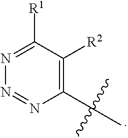

In some embodiments, the optionally substituted ring A is

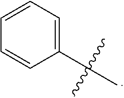 [SMILES: *c1ccccc1]

In some embodiments, the optionally substituted ring A is

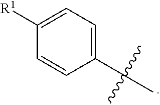

In some embodiments, the optionally substituted ring A is

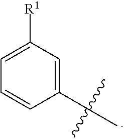

In some embodiments, the optionally substituted ring A is

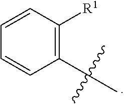

In some embodiments, the optionally substituted ring A is

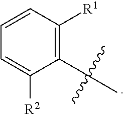

In some embodiments, the optionally substituted ring A is

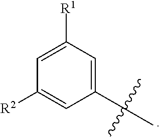

In some embodiments, the optionally substituted ring A is

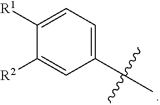

In some embodiments, the optionally substituted ring A is

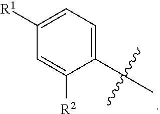

In some embodiments, the optionally substituted ring A is

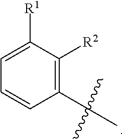

In some embodiments, the optionally substituted ring A is

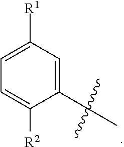

In some embodiments, the optionally substituted ring A is

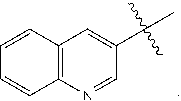 [SMILES: *c1cnc2ccccc2c1]

In some embodiments, the optionally substituted ring A is

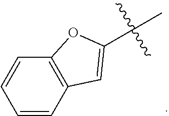 [SMILES: *c1cc2ccccc2o1]

In some embodiments, the optionally substituted ring A is

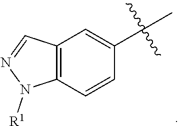

In some embodiments, the optionally substituted ring A is

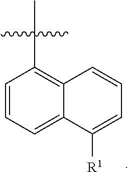

In some embodiments, the optionally substituted ring A is

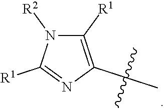

In some embodiments, the optionally substituted ring A is

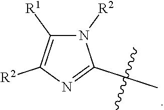

In some embodiments, the optionally substituted ring A is

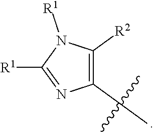

In some embodiments, the optionally substituted ring A is

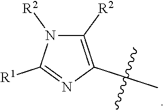

In some embodiments, the optionally substituted ring A is

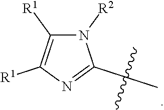

In some embodiments, the optionally substituted ring A is

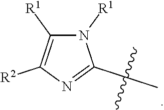

In some embodiments, the optionally substituted ring A is

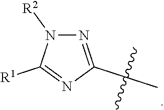

In some embodiments, the optionally substituted ring A is

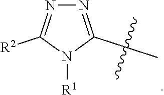

In some embodiments, the optionally substituted ring A is

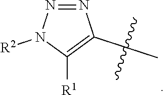

In some embodiments, the optionally substituted ring A is

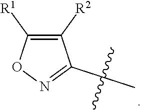

In some embodiments, the optionally substituted ring A is

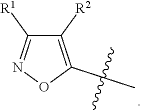

In some embodiments, the optionally substituted ring A is

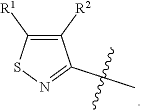

In some embodiments, the optionally substituted ring A is

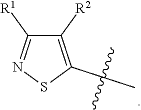

In some embodiments, the substituted ring A is

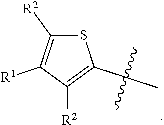

In some embodiments, the substituted ring A is

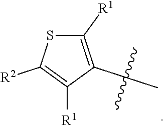

In some embodiments, the substituted ring A is

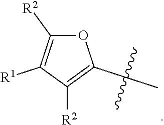

In some embodiments, the substituted ring A is

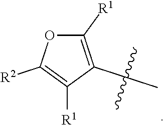

In some embodiments, the substituted ring A is

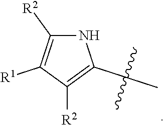

In some embodiments, the substituted ring A is

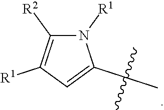

In some embodiments, the substituted ring A is

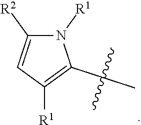

In some embodiments, the substituted ring A is

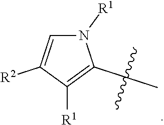

In some embodiments, the substituted ring A is

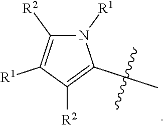

In some embodiments, the substituted ring A is

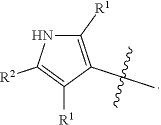

In some embodiments, the substituted ring A is

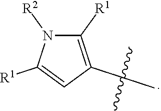

In some embodiments, the substituted ring A is

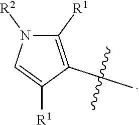

In some embodiments, the substituted ring A is

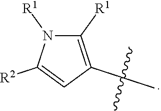

In some embodiments, the substituted ring A is

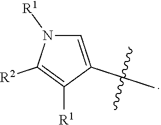

In some embodiments, the substituted ring A is

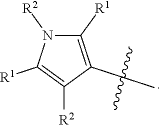

In some embodiments, the optionally substituted ring A is

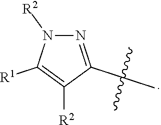

In some embodiments, the optionally substituted ring A is

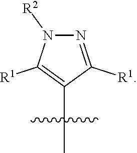

In some embodiments, the optionally substituted ring A is

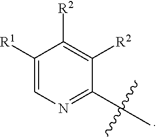

In some embodiments, the optionally substituted ring A is

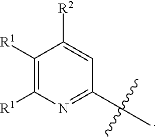

In some embodiments, the optionally substituted ring A is

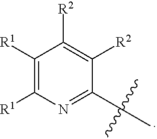

In some embodiments, the optionally substituted ring A is

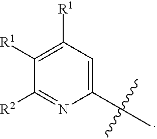

In some embodiments, the optionally substituted ring A is

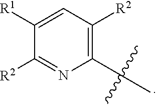

In some embodiments, the optionally substituted ring A is

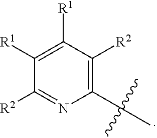

In some embodiments, the optionally substituted ring A is

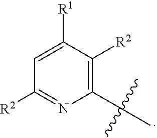

In some embodiments, the optionally substituted ring A is

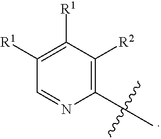

In some embodiments, the optionally substituted ring A is

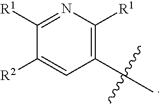

In some embodiments, the optionally substituted ring A is

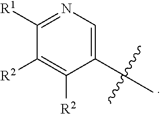

In some embodiments, the optionally substituted ring A is

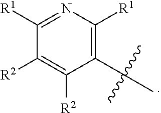

In some embodiments, the optionally substituted ring A is

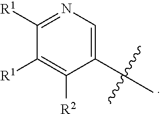

In some embodiments, the optionally substituted ring A is

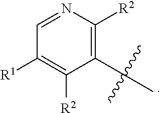

In some embodiments, the optionally substituted ring A is

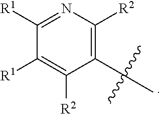

In some embodiments, the optionally substituted ring A is

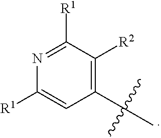

In some embodiments, the optionally substituted ring A is

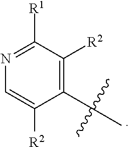

In some embodiments, the optionally substituted ring A is

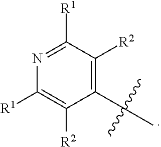

In some embodiments, the optionally substituted ring A is

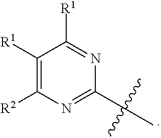

In some embodiments, the optionally substituted ring A is

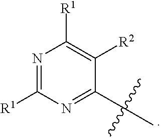

In some embodiments, the optionally substituted ring A is

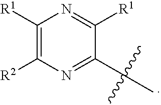

In some embodiments, the optionally substituted ring A is

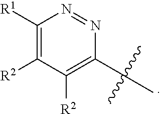

In some embodiments, the optionally substituted ring A is

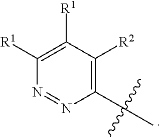

In some embodiments, the optionally substituted ring A is

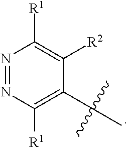

In some embodiments, the optionally substituted ring A is

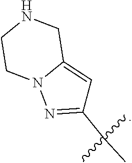 [SMILES: *c1cc2n(n1)CCNC2]

In some embodiments, the optionally substituted ring A is

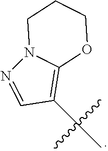 [SMILES: *c1cnn2c1OCCC2]

In some embodiments, the optionally substituted ring A is

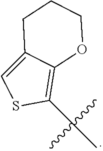 [SMILES: *c1scc2c1OCCC2]

In some embodiments, the optionally substituted ring A is

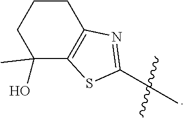 [SMILES: *c1nc2c(s1)C(C)(O)CCC2]

In some embodiments, the optionally substituted ring A is selected from the group consisting of:

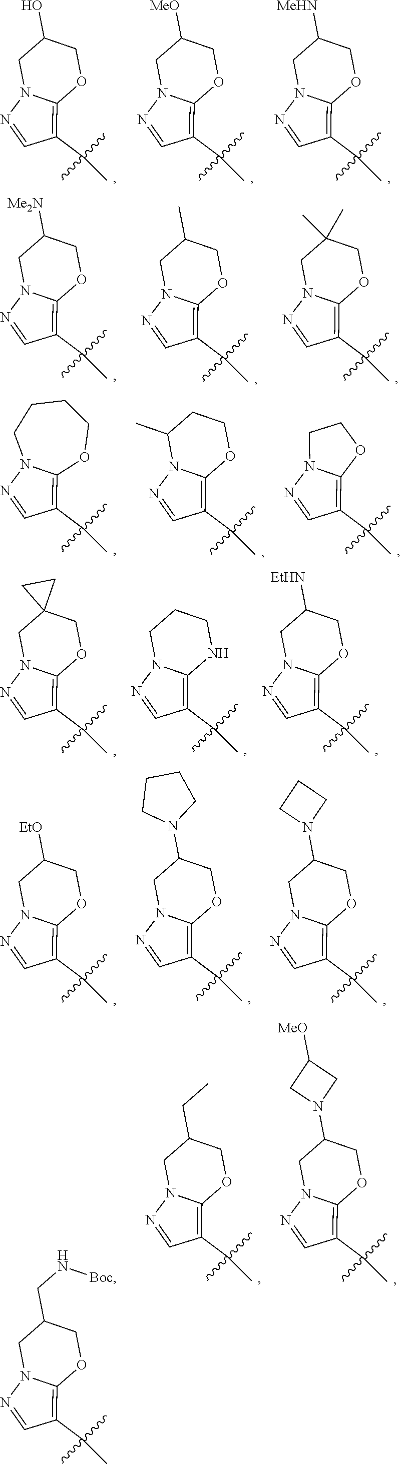

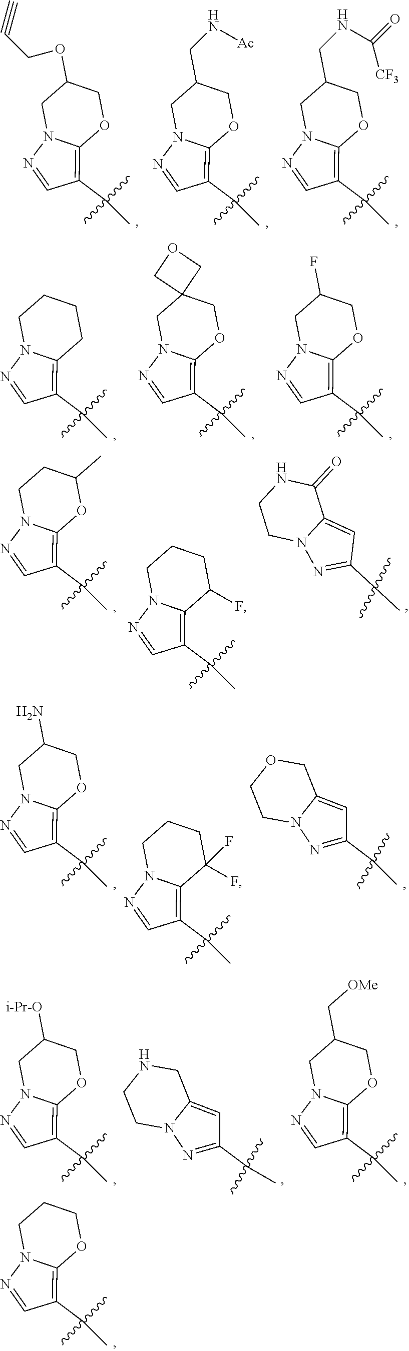

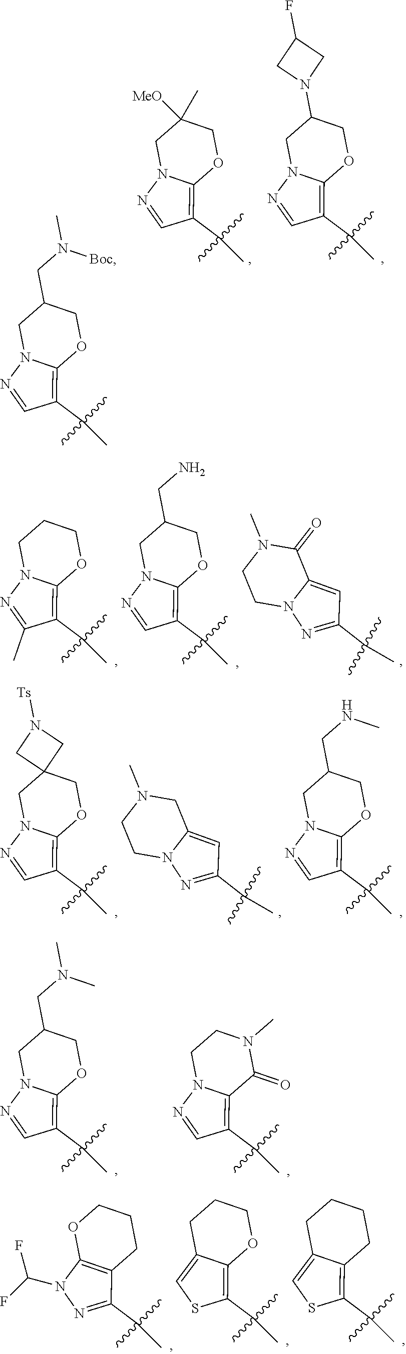

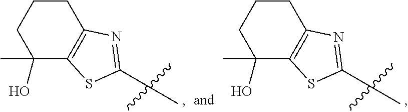 [SMILES: *c1nc2c(s1)C(C)(O)CCC2 ; *c1nc2c(s1)C(C)(O)CCC2]

In some embodiments, the optionally substituted ring A is selected from the group consisting of:

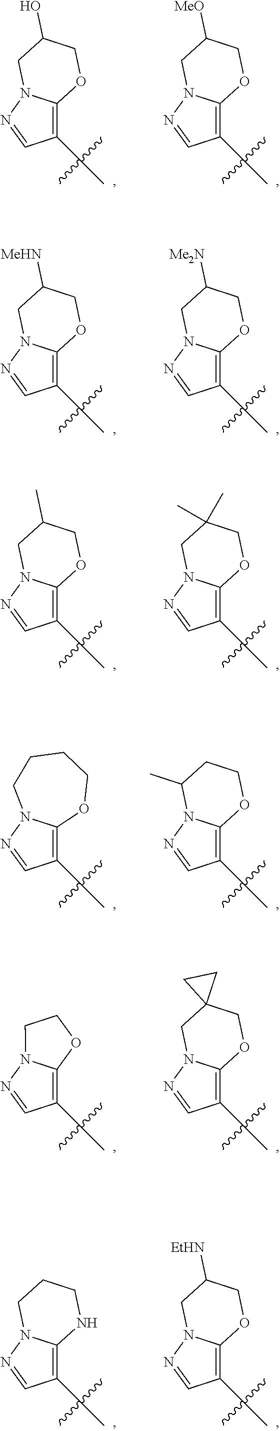

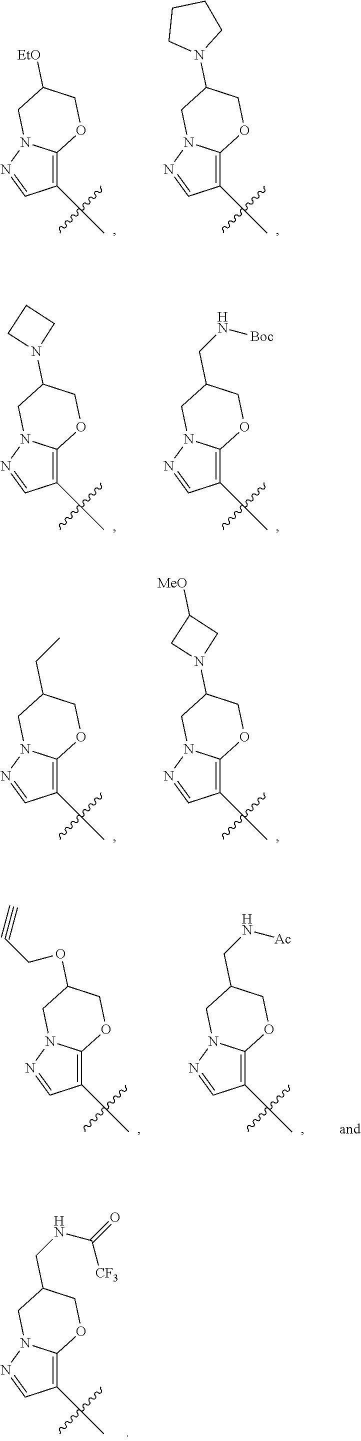

In some embodiments, the optionally substituted ring A is selected from the group consisting of:

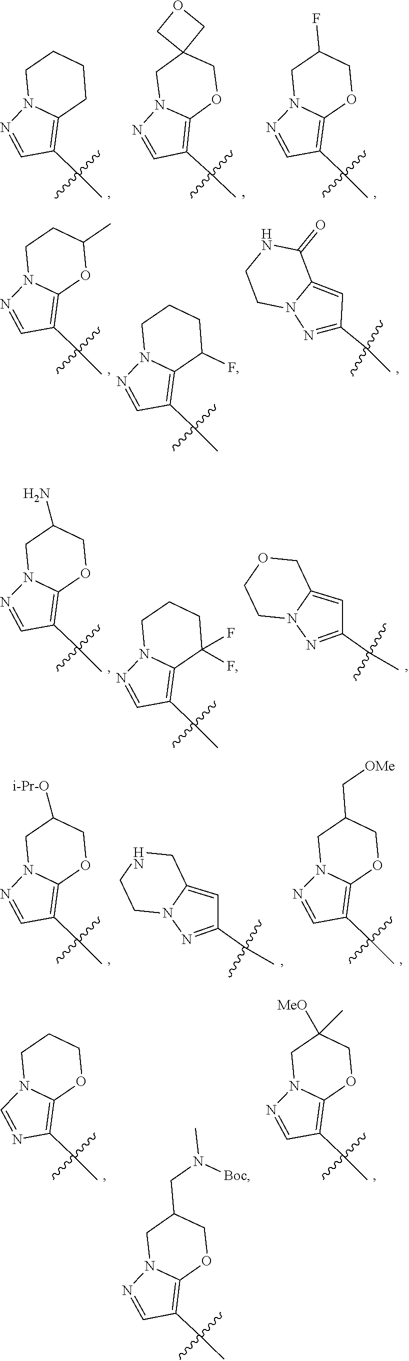

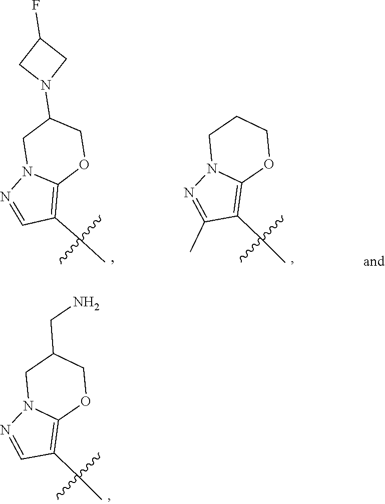 [SMILES: *c1cnn2c1OCC(N1CC(F)C1)C2 ; *c1c(C)nn2c1OCCC2 ; *c1cnn2c1OCC(CN)C2]

In some embodiments, the optionally substituted ring A is selected from the group consisting of:

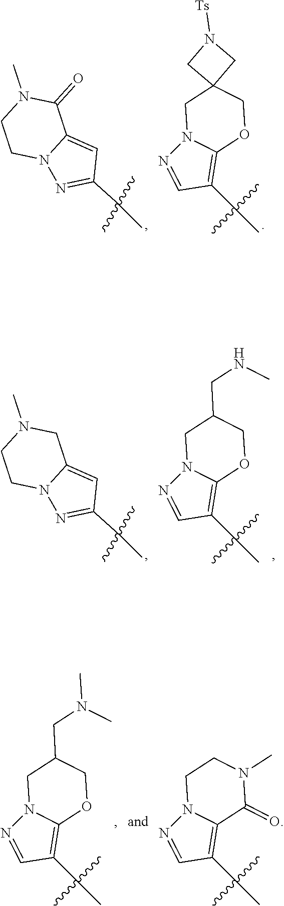 [SMILES: *c1cc2n(n1)CCN(C)C2=O ; *c1cnn2c1OCC1(CN(S(=O)(=O)c3ccc(C)cc3)C1)C2 ; *c1cc2n(n1)CCN(C)C2 ; *c1cnn2c1OCC(CNC)C2 ; *c1cnn2c1OCC(CN(C)C)C2 ; *c1cnn2c1C(=O)N(C)CC2]

In some embodiments, the optionally substituted ring A is selected from the group consisting of:

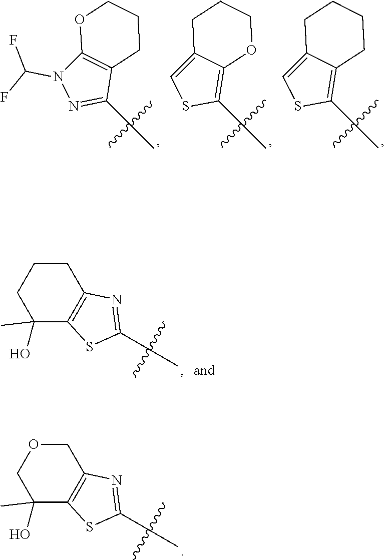

In some embodiments, the substituted A ring is selected from the group consisting of (A-1) to (A-51):

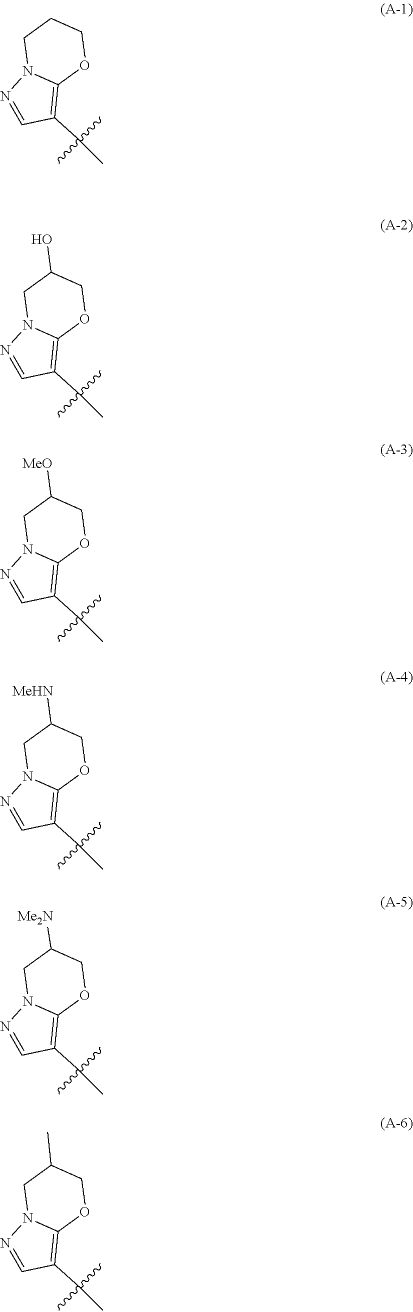 [SMILES: *c1cnn2c1OCCC2 ; *c1cnn2c1OCC(O)C2 ; *c1cnn2c1OCC(OC)C2 ; *c1cnn2c1OCC(NC)C2 ; *c1cnn2c1OCC(N(C)C)C2 ; *c1cnn2c1OCC(C)C2]

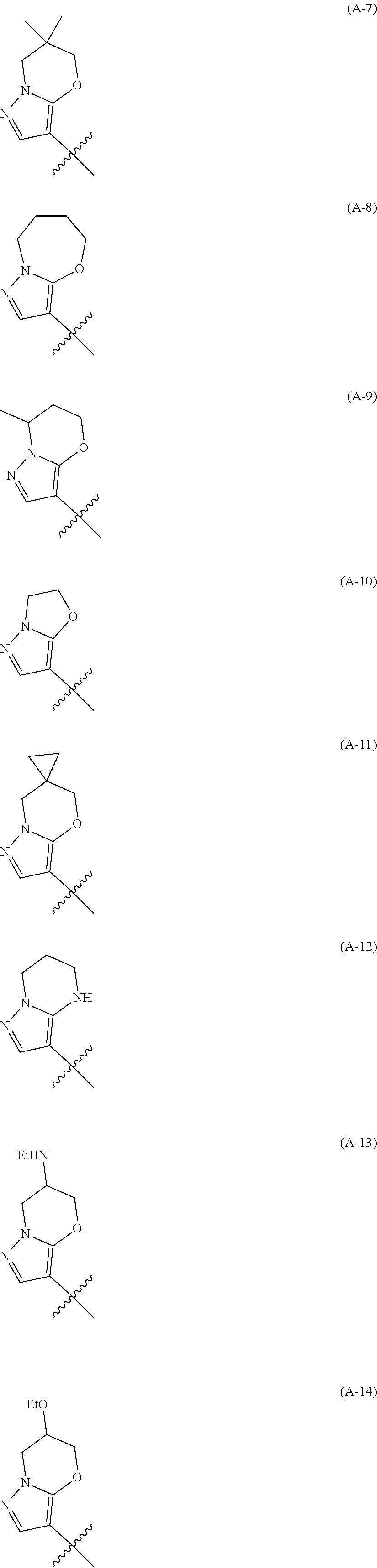

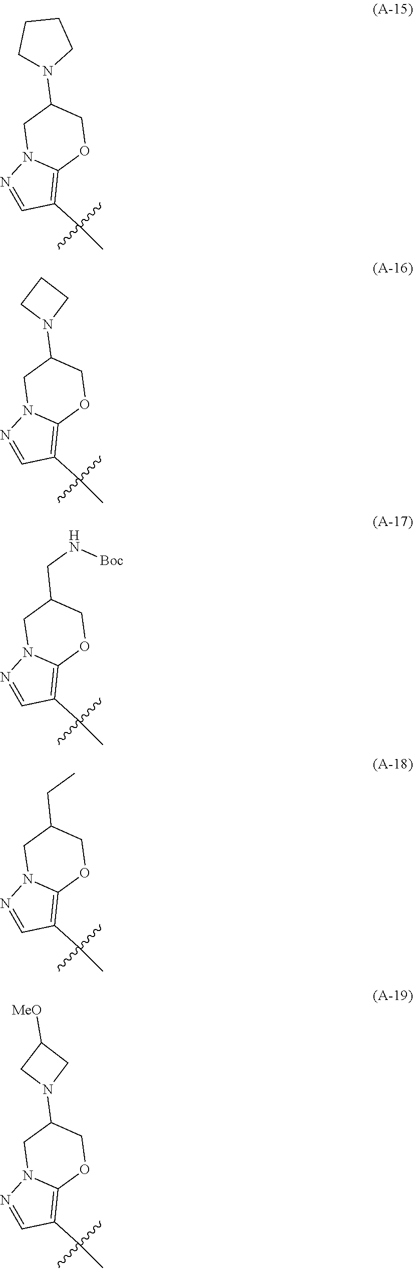 [SMILES: *c1cnn2c1OCC(N1CCCC1)C2 ; *c1cnn2c1OCC(N1CCC1)C2 ; *c1cnn2c1OCC(CNC(=O)OC(C)(C)C)C2 ; *c1cnn2c1OCC(CC)C2 ; *c1cnn2c1OCC(N1CC(OC)C1)C2]

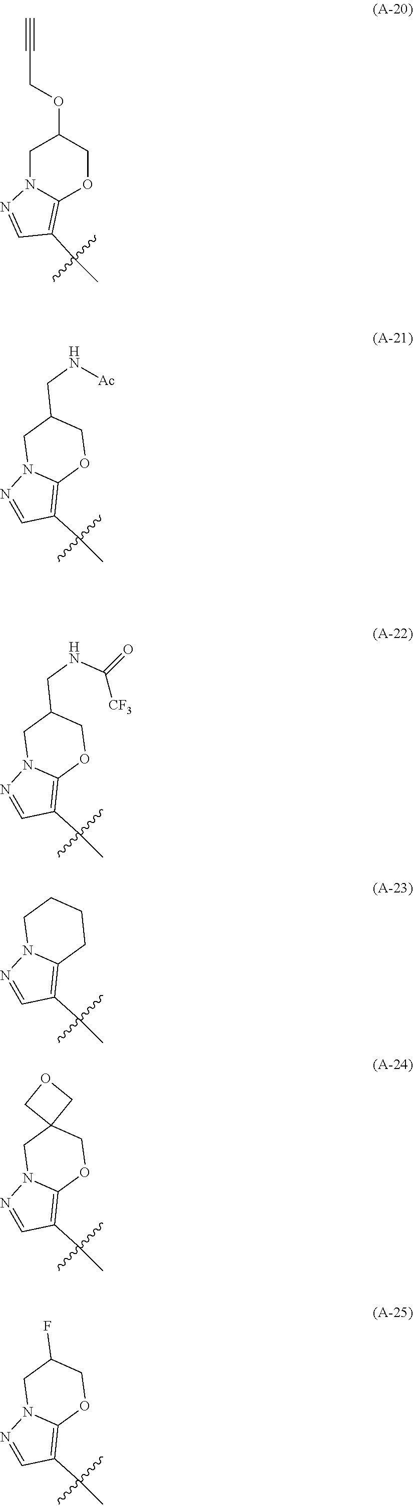 [SMILES: *c1cnn2c1OCC(OCC#C)C2 ; *c1cnn2c1OCC(CNC(C)=O)C2 ; *c1cnn2c1OCC(CNC(=O)C(F)(F)F)C2 ; *c1cnn2c1CCCC2 ; *c1cnn2c1OCC1(COC1)C2 ; *c1cnn2c1OCC(F)C2]

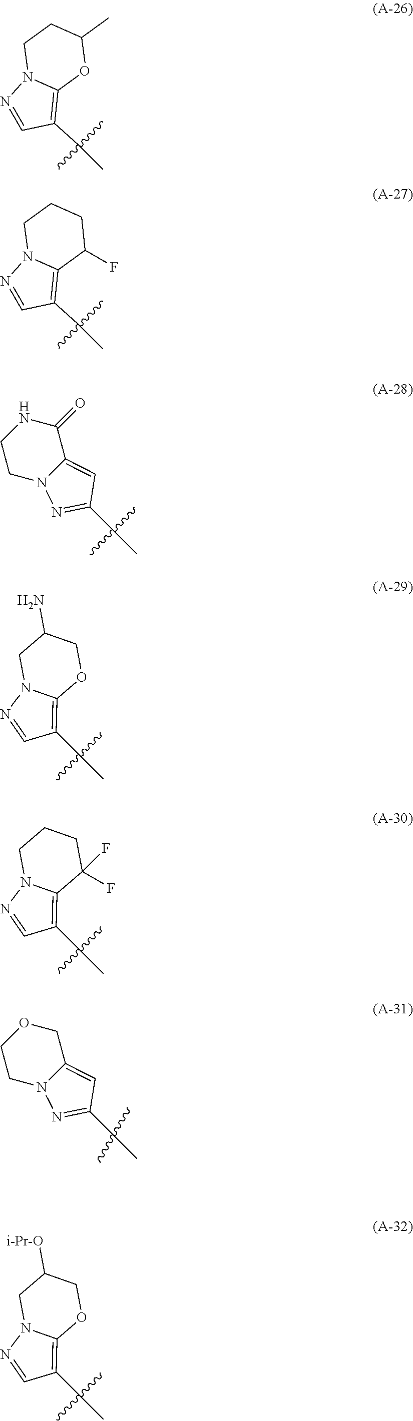

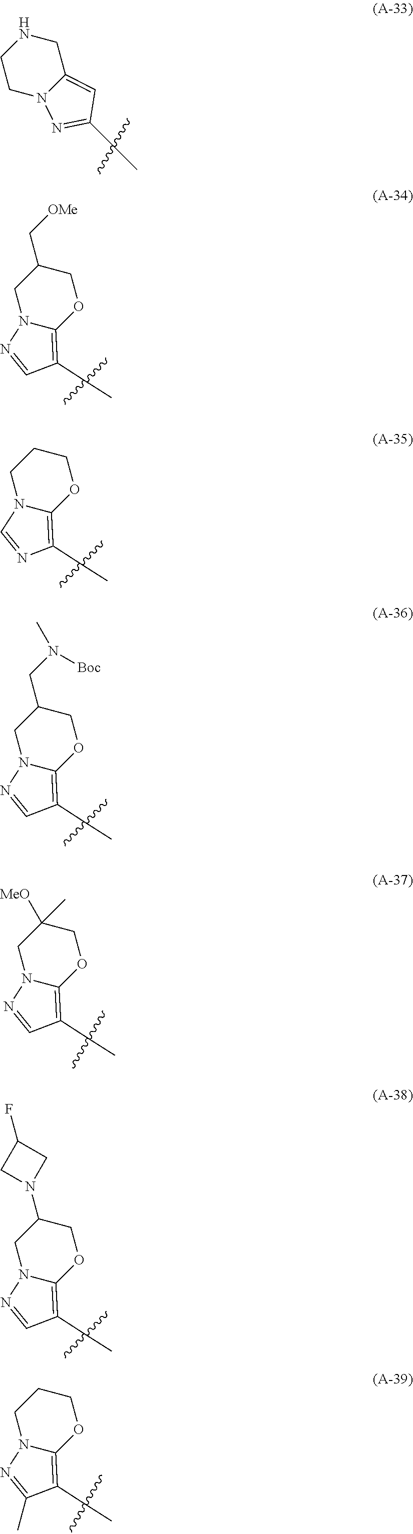

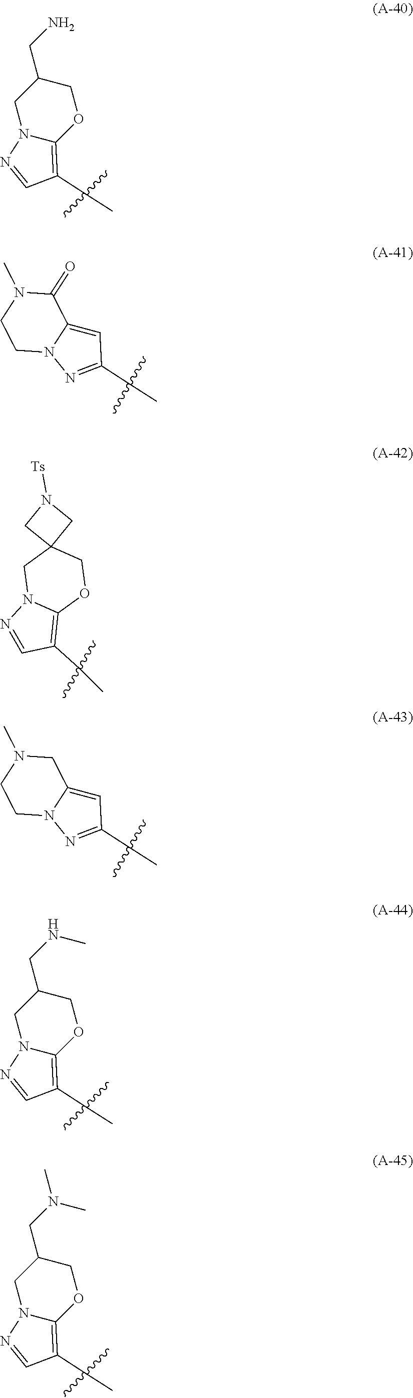 [SMILES: *c1cnn2c1OCC(CN)C2 ; *c1cc2n(n1)CCN(C)C2=O ; *c1cnn2c1OCC1(CN(S(=O)(=O)c3ccc(C)cc3)C1)C2 ; *c1cc2n(n1)CCN(C)C2 ; *c1cnn2c1OCC(CNC)C2 ; *c1cnn2c1OCC(CN(C)C)C2]

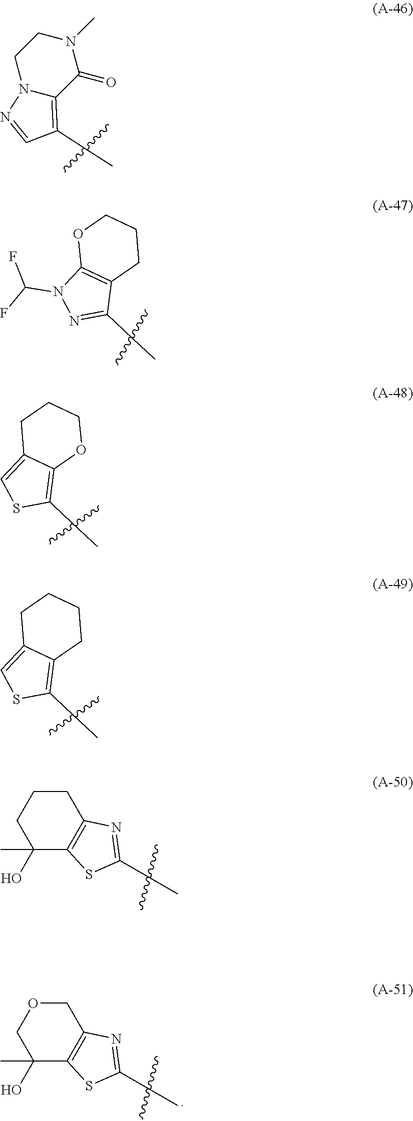 [SMILES: *c1cnn2c1C(=O)N(C)CC2 ; *c1nn(C(F)F)c2c1CCCO2 ; *c1scc2c1OCCC2 ; *c1scc2c1CCCC2 ; *c1nc2c(s1)C(C)(O)CCC2 ; *c1nc2c(s1)C(C)(O)COC2]

The Groups R¹ and R²

In some embodiments,

R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO-5- to 10-membered heteroaryl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, SC₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo; wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,

R¹ and R² are each independently selected from C₁-C₆ haloalkyl, C₁-C₆ alkoxy, C₁-C₆ haloalkoxy, halo, CN, NO₂, COC₁-C₆ alkyl, CO—C₆-C₁₀ aryl, CO-5- to 10-membered heteroaryl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₃-C₇ cycloalkyl, C₁-C₆ haloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo; wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,

R¹ and R² are each independently selected from C₁-C₆ alkyl, halo, CN, NO₂, COC₁-C₆ alkyl, CO—C₆-C₁₀ aryl, CO-5- to 10-membered heteroaryl, CO₂C₁-C₆ alkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo; wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,

R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO-5- to 10-membered heteroaryl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is unsubstituted; wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are unsubstituted;
   or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,

R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO-5- to 10-membered heteroaryl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments,

R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO-5- to 10-membered heteroaryl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl are each unsubstituted;
or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,

R¹ and R² are each independently selected from C₁-C₆ alkyl, halo, CN, COC₁-C₆ alkyl, CO₂C₁-C₆ alkyl, C₆-C₁₀ aryl, S(O)C₁-C₆ alkyl, 5- to 10-membered heteroaryl, and 3- to 7-membered heterocycloalkyl,
wherein the C₁-C₆ alkyl and 3- to 7-membered heterocycloalkyl is optionally substituted with one or more substituents each independently selected from hydroxy and oxo.

In some embodiments, each of R¹ and R², when present, is independently selected from the group consisting of C₁-C₆ alkyl optionally substituted with one or more hydroxy, halo, oxo, C₁-C₆ alkoxy, or NR⁸R⁹; C₃-C₇ cycloalkyl optionally substituted with one or more hydroxy, halo, oxo, C₁-C₆ alkoxy, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; 3- to 7-membered heterocycloalkyl optionally substituted with one or more hydroxy, halo, oxo, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; C₁-C₆ haloalkyl; C₁-C₆ alkoxy; C₁-C₆ haloalkoxy; halo; CN; CO—C₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl optionally substituted with one or more independently halo; 5- to 10-membered heteroaryl; NH₂; NHC₁-C₆ alkyl; N(C₁-C₆ alkyl)₂; CONR⁸R⁹; SF₅; S(O₂)NR¹¹R¹²; S(O)C₁-C₆ alkyl; and S(O₂)C₁-C₆ alkyl.

In some embodiments, each of R¹ and R² is independently selected from the group consisting of C₁-C₆ alkyl optionally substituted with one or more hydroxy, halo, oxo, C₁-C₆ alkoxy, or NR⁸R⁹; C₃-C₇ cycloalkyl optionally substituted with one or more hydroxy, halo, oxo, C₁-C₆ alkoxy, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; 3- to 7-membered heterocycloalkyl optionally substituted with one or more hydroxy, halo, oxo, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; C₁-C₆ haloalkyl; C₁-C₆ alkoxy; C₁-C₆ haloalkoxy; halo; CN; CO—C₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; S(O₂)NR¹¹R¹²; S(O)C₁-C₆ alkyl; and S(O₂)C₁-C₆ alkyl.

In some embodiments, R¹ is selected from the group consisting of 1-hydroxy-2-methylpropan-2-yl; methyl; isopropyl; 2-hydroxy-2-propyl; 1,2-dihydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; —COCH₃; COCH₂CH₃; 2-methoxy-2-propyl; difluoromethyl; (dimethylamino)methyl; (methylamino)methyl; 1-(dimethylamino)ethyl; fluoro; chloro; phenyl; fluorophenyl; pyridyl; pyrazolyl; S(O₂)CH₃; and S(O₂)NR¹¹R¹².

In some embodiments, R¹ is selected from the group consisting of 1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; S(O₂)CH₃; and S(O₂)NR¹¹R¹².

In some embodiments, R² is selected from the group consisting of fluoro; chloro; cyano; methyl; methoxy; ethoxy; isopropyl; 1-hydroxy-2-methylpropan-2-yl; 2-hydroxy-2-propyl; 1,2-dihydroxy-2-propyl; hydroxymethyl; 1-hydroxyethyl; 2-hydroxyethyl; 1-hydroxy-2-propyl; 1-hydroxy-1-cyclopropyl; COCH₃; COPh; 2-methoxy-2-propyl; difluoromethyl; (dimethylamino)methyl; (methylamino)methyl; S(O₂)CH₃; and S(O₂)NR¹¹R¹².

In some embodiments, R² is selected from the group consisting of fluoro, chloro, cyano, methyl; methoxy; ethoxy; isopropyl; 1-hydroxy-2-methylpropan-2-yl; 2-hydroxy-2-propyl; hydroxymethyl; 1-hydroxyethyl; 2-hydroxyethyl; 1-hydroxy-2-propyl; 1-hydroxy-1-cyclopropyl; COCH₃; COPh; 2-methoxy-2-propyl; (dimethylamino)methyl; S(O₂)CH₃; and S(O₂)NR¹¹R¹².

In some embodiments, one or more R¹ when present is independently a C₁-C₆ alkyl substituted with one or more hydroxy.

In certain of these embodiments, one or more R¹ is independently selected from 1-hydroxy-2-methylpropan-2-yl; 2-hydroxy-2-propyl; hydroxymethyl; 1-hydroxyethyl; 2-hydroxyethyl; 1-hydroxy-2-propyl; 1,2-dihydroxy-2-propyl; and 1,2,3-trihydroxy-2-propyl.

In some embodiments, one or more R¹ when present is independently a C₁-C₆ alkyl substituted with one or more hydroxy and further substituted with one or more (e.g., one) NR⁸R⁹.

In certain of these embodiments, one or more R¹ is independently selected from 1-amino-2-hydroxy-prop-2-yl; 1-acetamido-2-hydroxy-prop-2-yl; and 1-(tert-butoxycarbonyl)amino-2-hydroxy-prop-2-yl.

In some embodiments, one or more R¹ when present is independently a C₁-C₆ alkyl substituted with one or more hydroxy and further substituted with one or more (e.g., one) R¹⁵.

In certain of these embodiments (e.g., a2=1 or 2), one or more R¹ is independently selected from 1-(2-hydroxyethoxy)-2-hydroxy-2-propyl; 1-(2-benzyloxyethoxy)-2-hydroxy-2-propyl; and 1-(2-methoxyethoxy)-2-hydroxy-2-propyl.

In certain of these embodiments (e.g., a2=1), one or more R¹ is independently selected from 1-(2-hydroxyethoxy)-2-hydroxy-2-propyl and 1-(2-methoxyethoxy)-2-hydroxy-2-propyl.

In certain embodiments (e.g., a2=1), one or more R¹ is independently selected from:

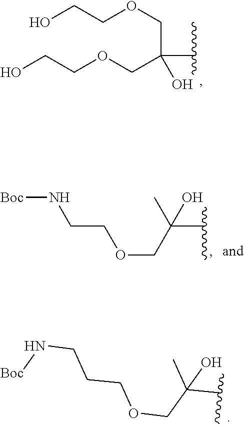 [SMILES: *C(O)(COCCO)COCCO ; *C(C)(O)COCCNC(=O)OC(C)(C)C ; *C(C)(O)COCCCNC(=O)OC(C)(C)C]

In certain embodiments (e.g., a2>1), one or more R¹ is

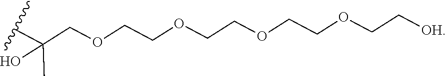 [SMILES: *C(C)(O)COCCOCCOCCOCCO]

In some embodiments, one or more R¹ is independently C₁-C₆ alkyl substituted with one or more (e.g., one) NR⁸R⁹ and further optionally substituted with one or more halo.

In certain of these embodiments, one or more R¹ is independently selected from: (methylamino)methyl; (2,2-difluoroeth-1-yl)(methyl)aminomethyl; (2,2,2-trifluoroeth-1-yl)(methyl)aminomethyl; (dimethylamino)methyl; 1-(dimethylamino)ethyl; 2-((methyl)aminomethyl)-prop-2-yl; 2-((methyl)amino)-prop-2-yl; (methyl)(cyclopropylmethyl)aminomethyl; (methyl)(2-(dimethylamino)eth-1-yl)aminomethyl; (cyclobutyl)(methyl)aminomethyl; 1-(cyclobutyl)amino-eth-1-yl; isopropylaminomethyl; (cyclobutyl)aminomethyl; cycloheptylaminomethyl; tetrahydropyranylaminomethyl; sec-butylaminomethyl; ethylaminomethyl; allylaminomethyl; (2,2-difluoroeth-1-yl)aminomethyl; (2-methoxy-eth-1-yl)aminomethyl; (2-methoxy-eth-1-yl)(methyl)aminomethyl; 2-fluoro-1-dimethylamino-eth-1-yl; 1-dimethylamino-2,2-difluoroeth-1-yl; 1-dimethylamino-2,2,2-trifluoroeth-1-yl; 1-dimethylamino-2,2,2-trimethyleth-1-yl; and dimethylamino(cyclopropyl)methyl (e.g., one or more R¹ is dimethylaminomethyl or methylaminomethyl).

In some embodiments, one or more R¹ is C₁-C₆ alkyl that is optionally substituted with one or more halo. In certain of these embodiments, one or more R¹ is C₂-C₆ alkyl that is optionally substituted with one or more halo. As non-limiting examples, R¹ is ethyl or difluoromethyl.

In certain of any of the foregoing embodiments of R¹, one or more R² is independently selected from C₁-C₆ alkyl, C₁-C₆ alkyl optionally substituted with one or more hydroxy, C₁-C₆ alkyl optionally substituted with one or more C₁-C₆ alkoxy, and halo.

In some embodiments, one pair of R¹ and R² is on adjacent atoms, and taken together with the atoms connecting them, independently form one ring selected from:

(a) monocyclic or bicyclic C₄-C₁₂ carbocyclic ring optionally substituted with one or more substituents independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;

(b) monocyclic or bicyclic 5-to-12-membered non-aromatic heterocyclic ring containing 1-3 heteroatoms independently selected from O, N, and S, wherein the heterocyclic ring is optionally substituted with one or more substituents independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;

(c) monocyclic or bicyclic 6-to-12-membered aromatic heterocyclic ring containing 1-3 heteroatoms independently selected from O, N, and S, wherein the heterocyclic ring is optionally substituted with one or more substituents independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;

(d) monocyclic 5-membered aromatic heterocyclic ring containing 2 heteroatoms independently selected from O, N, and S, wherein the heterocyclic ring is substituted with one substituent selected from hydroxy, halo, oxo, C₂-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;

(e) monocyclic 5-membered aromatic heterocyclic ring containing 2 heteroatoms independently selected from O, N, and S, wherein the heterocyclic ring is optionally substituted with two or more substituents independently selected from hydroxy, halo, oxo, C₂-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹; and

(f) monocyclic 5-membered aromatic heterocyclic ring containing 1 or 3 heteroatoms independently selected from O, N, and S, wherein the heterocyclic ring is optionally substituted with two or more substituents independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, one pair of R¹ and R² is on adjacent atoms, and taken together with the atoms connecting them, independently form one monocyclic or bicyclic C₄-C₁₂ non-aromatic carbocyclic ring or one monocyclic or bicyclic 5-to-12-membered non-aromatic heterocyclic ring containing 1-3 heteroatoms independently selected from O, N, and S, wherein the carbocyclic ring or heterocyclic ring is optionally independently substituted with one or more substituents independently selected from hydroxy, halo, oxo, C₁-C₆ alkyl, C₂-C₆ alkenyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹′ wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, oxo, NR⁸R⁹, ═NR¹⁰, COOC₁-C₆ alkyl, C₆-C₁₀ aryl, and CONR⁸R⁹.

In some embodiments, when a pair of R¹ and R² on adjacent atoms, taken together with the atoms connecting them, independently form one C₄-C₈ carbocyclic ring or one 5-to-8-membered heterocyclic ring containing 1 or 2 heteroatoms independently selected from O, N, and S, then the carbocyclic ring or heterocyclic ring is independently substituted with one or more substituents each independently selected from from C₂-C₆ alkenyl, C₂-C₆ alkynyl, OC₃-C₁₀ cycloalkyl, CN, OS(O₂)C₆-C₁₀ aryl, S(O₂)C₆-C₁₀ aryl, 5- to 10-membered heteroaryl, C₃-C₁₀ cycloalkyl, and 3- to 10-membered heterocycloalkyl, wherein the S(O₂)C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, one pair of R¹ and R² is on adjacent atoms, and taken together with the atoms connecting them, independently form one monocyclic or bicyclic C₄-C₁₂ carbocyclic ring (e.g., C₅ or C₆ carbocyclic ring) or one monocyclic or bicyclic 5-to-12-membered heterocyclic ring containing 1-3 (e.g., 1-2, e.g., 2) heteroatoms independently selected from O, N, and S (e.g., tetrahydropyridine, dihydrofuran, or dihydropyran), wherein the carbocyclic ring or heterocyclic ring is optionally independently substituted with one or more substituents each independently selected from hydroxy, halo, oxo, C₁-C₆ alkyl (e.g., methyl), C₂-C₆ alkenyl, C₂-C₆ alkynyl, C₁-C₆ alkoxy (e.g., methoxy, ethoxy, isopropoxyl), OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl (e.g., azetidinyl or oxetanyl), and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo (e.g., fluoro), C₁-C₆ alkyl, C₂-C₆ alkenyl, C₂-C₆ alkynyl, C₃-C₁₀ cycloalkyl, C₁-C₆ alkoxy, oxo, NR⁸R⁹ (e.g., amino, methylamino, or dimethylamino), ═NR¹⁰, COOC₁-C₆ alkyl, C₆-C₁₀ aryl, and CONR⁸R⁹.

In some embodiments, one pair of R¹ and R² is on adjacent atoms, and taken together with the atoms connecting them, independently form one monocyclic or bicyclic C₅-C₆ carbocyclic ring wherein the carbocyclic ring is optionally substituted with one or more substituents each independently selected from hydroxy, halo, oxo, methyl, isopropoxyl, azetidinyl, oxetanyl, wherein the methyl, isopropoxyl, azetidinyl, and oxetanyl are optionally substituted with one or more substituents each independently selected from hydroxy, fluoro, amino, methylamino, and dimethylamino; or

one pair of R¹ and R² on adjacent atoms taken together forms a moiety selected from:

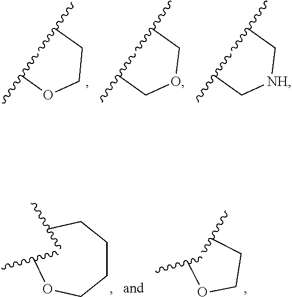

each of which is optionally independently substituted with one or more substituents each independently selected from hydroxy, halo, oxo, methyl, isopropoxyl, azetidinyl, oxetanyl, wherein the methyl, isopropoxyl, azetidinyl, and oxetanyl are optionally substituted with one or more substituents each independently selected from hydroxy, fluoro, amino, methylamino, and dimethylamino.

In some embodiments, one pair of R¹ and R² is on adjacent atoms, and taken together with the atoms connecting them, independently form at least one bicyclic spirocyclic C₄-C₁₂ carbocyclic ring, wherein the carbocyclic ring is optionally substituted with one or more substituents each independently selected from hydroxy, halo, oxo, methyl, isopropoxyl, azetidinyl, oxetanyl, wherein the methyl, isopropoxyl, azetidinyl, and oxetanyl are optionally substituted with one or more substituents each independently selected from hydroxy, fluoro, amino, methylamino, and dimethylamino.

In some embodiments, one pair of R¹ and R² is on adjacent atoms, and taken together with the atoms connecting them, independently form at least one bicyclic spirocyclic 5-to-12-membered heterocyclic ring containing 1-3 heteroatoms independently selected from O, N, and S, wherein the carbocyclic or heterocyclic ring is optionally substituted with one or more substituents each independently selected from hydroxy, halo, oxo, methyl, isopropoxyl, azetidinyl, oxetanyl, wherein the methyl, isopropoxyl, azetidinyl, and oxetanyl are optionally substituted with one or more substituents each independently selected from hydroxy, fluoro, amino, methylamino, and dimethylamino.

In some embodiments of the compound of Formula AA, when ring A is phenyl, then R¹ and R² are each independently selected from C₃ alkyl, C₅-C₆ alkyl, C₁-C₂ alkyl, tert-butyl, n-butyl, sec-butyl, iso-butyl, C₁-C₆ haloalkyl, C₁-C₆ alkoxy, C₁-C₆ haloalkoxy, F, I, CN, NO₂, COC₂-C₆ alkyl, CO—C₆-C₁₀ aryl, CO(5- to 10-membered heteroaryl), CO₂C₁-C₆ alkyl, CO₂C₃-C₈ cycloalkyl, OCOC₂-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,

wherein the C₃ alkyl, C₅-C₆ alkyl, C₁-C₆ haloalkyl, C₃-C₇ cycloalkyl and 3- to 7-membered heterocycloalkyl are optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;

wherein the C₁-C₂ alkyl, tert-butyl is substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;

wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;

wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or one pair of R¹ and R² on adjacent atoms, taken together with the atoms connecting them, independently form one monocyclic or bicyclic C₄ or C₆-C₁₂ carbocyclic ring or one monocyclic or bicyclic 5-to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, m=1; n=0; and

R¹ is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO-5- to 10-membered heteroaryl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, m=1; n=0; and,

R¹ and R² are each independently selected from C₁-C₆ alkyl, halo, CN, COC₁-C₆ alkyl, CO₂C₁-C₆ alkyl, C₆-C₁₀ aryl, 5- to 10-membered heteroaryl, S(O)C₁-C₆ alkyl, and 3- to 7-membered heterocycloalkyl,
wherein the C₁-C₆ alkyl and 3- to 7-membered heterocycloalkyl is optionally substituted with one or more substituents each independently selected from hydroxy and oxo.

In some embodiments, m=1; n=0; and,

R¹ is selected from C₁-C₆ alkyl, halo, CN, COC₁-C₆ alkyl, CO₂C₁-C₆ alkyl, C₆-C₁₀ aryl, 5- to 10-membered heteroaryl, S(O)C₁-C₆ alkyl, and 3- to 7-membered heterocycloalkyl, wherein the C₁-C₆ alkyl and 3- to 7-membered heterocycloalkyl is optionally substituted with one or more substituents each independently selected from hydroxy and oxo.

In some embodiments, m=1; n=1; and

R¹ and R² are each independently selected from C₁-C₆ alkyl, C₁-C₆ haloalkyl, C₁-C₆ alkoxy, C₁-C₆ haloalkoxy, halo, CN, NO₂, CO—C₆-C₁₀ aryl, CO-5- to 10-membered heteroaryl, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, m=1; n=1; and,

R¹ and R² are each independently selected from C₁-C₆ alkyl, halo, CN, COC₁-C₆ alkyl, CO₂C₁-C₆ alkyl, C₆-C₁₀ aryl, 5- to 10-membered heteroaryl, S(O)C₁-C₆ alkyl, and 3- to 7-membered heterocycloalkyl,
wherein the C₁-C₆ alkyl and 3- to 7-membered heterocycloalkyl is optionally substituted with one or more substituents each independently selected from hydroxy and oxo.

In some embodiments, m=1; n=1; and

R¹ and R² are on adjacent atoms, and taken together with the atoms connecting them, form a C₄-C₈ carbocyclic ring or a 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, m=1; n=1; and

R¹ and R² are on adjacent atoms, and taken together with the atoms connecting them, form a C₅-C₈ carbocyclic ring or a 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, m=1; n=1; and

R¹ and R² are on adjacent atoms, and taken together with the atoms connecting them, form a C₆ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, m=1; n=1; and

R¹ and R² are on adjacent atoms, and taken together with the atoms connecting them, form a C₅ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, m=1; n=1; and

R¹ and R² are on adjacent atoms, and taken together with the atoms connecting them, form a C₅-C₈ carbocyclic ring or a 5-to-8-membered heterocyclic ring containing 1 or 2 heteroatoms independently selected from O, N, and S, wherein the carbocyclic ring or heterocyclic ring is unsubstituted.

In some embodiments, m=1; n=1; and

R¹ and R² are on adjacent atoms, and taken together with the atoms connecting them, form a C₅-C₈ carbocyclic ring or a 5-to-8-membered heterocyclic ring containing 1 or 2 heteroatoms independently selected from O, N, and S, wherein the carbocyclic ring or heterocyclic ring is unsubstituted.

### Particular Embodiments Wherein m=1 and n=0

In some embodiments, R¹ is C₁-C₆ alkyl optionally substituted with one or more hydroxy.

In certain of these embodiments, R¹ is independently selected from 1-hydroxy-2-methylpropan-2-yl; 2-hydroxy-2-propyl; hydroxymethyl; 1-hydroxyethyl; 2-hydroxyethyl; 1-hydroxy-2-propyl; 1,2-dihydroxy-2-propyl; and 1,2,3-trihydroxy-2-propyl.

In some embodiments, R¹ is 1-hydroxy-2-methylpropan-2-yl.

In some embodiments, R¹ is 2-hydroxy-2-propyl.

In some embodiments, R¹ is hydroxymethyl.

In some embodiments, R¹ is 1-hydroxyethyl.

In some embodiments, R¹ is 1-hydroxy-2-propyl.

In some embodiments, R¹ is 2-hydroxyethyl.

In some embodiments, R¹ is 1,2-dihydroxy-2-propyl.

In some embodiments, R¹ is 1,2,3-trihydroxy-2-propyl.

In some embodiments, R¹ is C₁-C₆ alkyl.

In some embodiments, R¹ is methyl.

In some embodiments, R¹ is isopropyl.

In some embodiments, R¹ is isobutyl.

In some embodiments, R¹ is C₁-C₆ alkyl substituted with hydroxy at the carbon directly connected to ring A.

In some embodiments, R¹ is 2-hydroxy-2-propyl.

In some embodiments, R¹ is hydroxymethyl.

In some embodiments, R¹ is 1-hydroxyethyl.

In some embodiments, R¹ is 2-hydroxyethyl.

In some embodiments, R¹ is 1-hydroxy-2-methyl-prop-2-yl.

In some embodiments, R¹ is 1-hydroxy-2-propyl.

In some embodiments, R¹ is 1,2-dihydroxy-2-propyl.

In some embodiments, R¹ is C₁-C₆ alkyl substituted with two or more hydroxy groups.

In some embodiments, R¹ is 1,2-dihydroxy-2-propyl.

In some embodiments, R¹ is 1,2-dihydroxy-3-propyl.

In some embodiments, R¹ is 1,3-dihydroxy-2-methyl-prop-2-yl.

In some embodiments, R¹ is 1,2,3-trihydroxy-prop-2-yl.

In some embodiments, R¹ is a C₁-C₆ alkyl substituted with one or more hydroxy and further substituted with one or more (e.g., one) NR⁸R⁹.

In certain of these embodiments, R¹ is independently selected from 1-amino-2-hydroxy-prop-2-yl; 1-acetamido-2-hydroxy-prop-2-yl; and 1-(tert-butoxycarbonyl)amino-2-hydroxy-prop-2-yl.

In some embodiments, R¹ is 1-amino-2-hydroxy-prop-2-yl.

In some embodiments, R¹ is 1-acetamido-2-hydroxy-prop-2-yl.

In some embodiments, R¹ is 1-(tert-butoxycarbonyl)amino-2-hydroxy-prop-2-yl.

In some embodiments, R¹ is independently a C₁-C₆ alkyl substituted with one or more hydroxy and further substituted with one or more (e.g., one) R¹⁵.

In certain of these embodiments, a2 is 1 in R¹⁵.

In certain of the foregoing embodiments, one or more R¹ is independently selected from 1-(2-hydroxyethoxy)-2-hydroxy-2-propyl; 1-(2-benzyloxyethoxy)-2-hydroxy-2-propyl; and 1-(2-methoxyethoxy)-2-hydroxy-2-propyl.

In certain embodiments, R¹ is

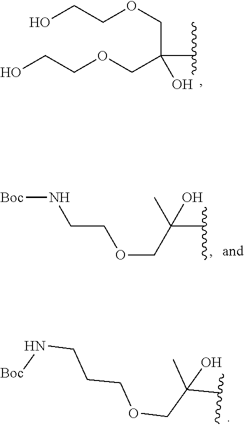 [SMILES: *C(O)(COCCO)COCCO ; *C(C)(O)COCCNC(=O)OC(C)(C)C ; *C(C)(O)COCCCNC(=O)OC(C)(C)C]

In certain embodiments when R¹ is independently a C₁-C₆ alkyl substituted with one or more hydroxy and further substituted with one or more (e.g., one) R¹⁵, a2 is >1.

In certain of these embodiments, R¹ is:

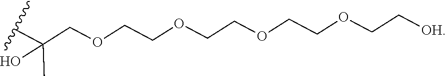 [SMILES: *C(C)(O)COCCOCCOCCOCCO]

In some embodiments, R¹ is C₁-C₆ alkyl optionally substituted with one or more R¹⁵.

In some embodiments, R¹ is C₃-C₇ cycloalkyl optionally substituted with one or more hydroxy.

In some embodiments, R¹ is C₃-C₇ cycloalkyl.

In some embodiments, R¹ is C₃-C₇ cycloalkyl substituted with hydroxy at the carbon directly connected to ring A.

In some embodiments, R¹ is 1-hydroxy-1-cyclopropyl.

In some embodiments, R¹ is 1-hydroxy-1-cyclobutyl.

In some embodiments, R¹ is 1-hydroxy-1-cyclopentyl.

In some embodiments, R¹ is 1-hydroxy-1-cyclohexyl.

In some embodiments, R¹ is 3- to 7-membered heterocycloalkyl optionally substituted with one or more hydroxy. In certain of these embodiments, R¹ is further optionally substituted with one or more C₁-C₆ alkyl, wherein each of said C₁-C₆ alkyl is further optionally substituted as defined anywhere herein. As a non-limiting example, R¹ is

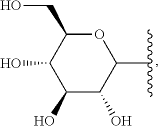 [SMILES: *C1O[C@H](CO)[C@@H](O)[C@H](O)[C@H]1O]

In some embodiments, R¹ is 3- to 7-membered heterocycloalkyl optionally substituted with one or more substituents independently selected from hydroxy and R¹⁵. In certain of these embodiments, R¹ is further optionally substituted with one or more C₁-C₆ alkyl, wherein each of said C₁-C₆ alkyl is further optionally substituted as defined anywhere herein. As a non-limiting example, R¹ is

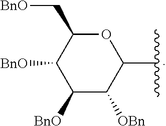 [SMILES: *C1O[C@H](COCc2ccccc2)[C@@H](OCc2ccccc2)[C@H](OCc2ccccc2)[C@H]1OCc1ccccc1]

In some embodiments, R¹ is 3- to 7-membered heterocycloalkyl.

In some embodiments, R¹ is morpholinyl (e.g., 4-morpholinyl).

In some embodiments, R¹ is azetidinyl.

In some embodiments, R¹ is 1,3-dioxolan-2-yl.

In some embodiments, R¹ is 3- to 7-membered heterocycloalkyl substituted with hydroxy at the carbon directly connected to ring A.

In some embodiments, R¹ is C₁-C₆ alkyl optionally substituted with one or more oxo.

In some embodiments, R¹ is COCH₃.

In some embodiments, R¹ is COCH₂CH₃.

In some embodiments, R¹ is C₃-C₇ cycloalkyl optionally substituted with one or more oxo.

In some embodiments, R¹ is 3- to 7-membered heterocycloalkyl optionally substituted with one or more oxo.

In some embodiments, R¹ is C₁-C₆ alkyl optionally substituted with one or more C₁-C₆ alkoxy.

In some embodiments, R¹ is 2-methoxy-2-propyl.

In some embodiments, R¹ is C₃-C₇ cycloalkyl optionally substituted with one or more C₁-C₆ alkoxy.

In some embodiments, R¹ is 3- to 7-membered heterocycloalkyl optionally substituted with one or more C₁-C₆ alkoxy.

In some embodiments, R¹ is 3- to 7-membered heterocycloalkyl optionally substituted with one or more oxo and further optionally substituted with one or more C1-C6 alkyl.

In some embodiments, R¹ is 5-methyl-oxazolidin-2-one-5-yl.

In some embodiments, R¹ is C₁-C₆ alkyl optionally substituted with one or more NR⁸R⁹.

In some embodiments, R¹ is (dimethylamino)methyl.

In some embodiments, R¹ is (methylamino)methyl.

In some embodiments, R¹ is 2-(dimethylamino)prop-2-yl.

In some embodiments, R¹ is aminomethyl.

In some embodiments, R¹ is N-methylacetamidomethyl.

In some embodiments, R¹ is 1-(dimethylamino)eth-1-yl.

In some embodiments, R¹ is 2-(dimethylamino)prop-2-yl.

In some embodiments, R¹ is (2-methoxy-eth-1-yl)(methyl)aminomethyl.

In some embodiments, R¹ is (methyl)(acetyl)aminomethyl.

In some embodiments, R¹ is (methyl)(cyclopropylmethyl)aminomethyl.

In some embodiments, R¹ is (methyl)(2,2-difluoroeth-1-yl)aminomethyl.

In some embodiments, R¹ is (2,2,2-trifluoroeth-1-yl)(methyl)aminomethyl.

In some embodiments, R¹ is 2-((methyl)aminomethyl)-prop-2-yl.

In some embodiments, R¹ is 2-((methyl)amino)-prop-2-yl.

In some embodiments, R¹ is (methyl)(cyclopropylmethyl)aminomethyl.

In some embodiments, R¹ is (methyl)(2-(dimethylamino)eth-1-yl)aminomethyl.

In some embodiments, R¹ is (cyclobutyl)(methyl)aminomethyl.

In some embodiments, R¹ is (2-methoxy-eth-1-yl)(methyl)aminomethyl.

In some embodiments, R¹ is 2-fluoro-1-dimethylamino-eth-1-yl.

In some embodiments, R¹ is 1-dimethylamino-2,2-difluoroeth-1-yl.

In some embodiments, R¹ is 1-dimethylamino-2,2,2-trifluoroeth-1-yl.

In some embodiments, R¹ is 1-dimethylamino-2,2,2-trimethyleth-1-yl.

In some embodiments, R¹ is (cyclobutyl)(methyl)aminomethyl.

In some embodiments, R¹ is isopropylaminomethyl.

In some embodiments, R¹ is (cyclobutyl)aminomethyl.

In some embodiments, R¹ is cycloheptylaminomethyl.

In some embodiments, R¹ is tetrahydropyranylaminomethyl.

In some embodiments, R¹ is sec-butylaminomethyl.

In some embodiments, R¹ is ethylaminomethyl.

In some embodiments, R¹ is allylaminomethyl.

In some embodiments, R¹ is 2,2-difluoroeth-1-yl)aminomethyl.

In some embodiments, R¹ is (2-methoxy-eth-1-yl)aminomethyl.

In some embodiments, R¹ is C₁-C₆ alkyl substituted with NR⁸R⁹, wherein said C₁-C₆ alkyl is further optionally substituted as described elsewhere herein.

In some embodiments, R¹ is dimethylamino(cyclopropyl)methyl.

In some embodiments, R¹ is C₃-C₇ cycloalkyl optionally substituted with one or more NR⁸R⁹.

In some embodiments, R¹ is 3- to 7-membered heterocycloalkyl optionally substituted with one or more NR⁸R⁹.

In some embodiments, R¹ is C₁-C₆ alkyl optionally substituted with one or more hydroxy and one or more oxo.

In some embodiments, R¹ is C₁-C₆ alkyl optionally substituted with one or more halo.

In some embodiments, R¹ is difluoromethyl.

In some embodiments, R¹ is C(Me)₂C(O)OH.

In some embodiments, R¹ is C₁-C₆ haloalkyl optionally substituted with one or more hydroxy.

In some embodiments, R¹ is C₁-C₆ alkoxy.

In some embodiments, R¹ is C₁-C₆ haloalkoxy.

In some embodiments, R¹ is halo.

In some embodiments, R¹ is fluoro.

In some embodiments, R¹ is chloro.

In some embodiments, R¹ is CN.

In some embodiments, R¹ is NO₂.

In some embodiments, R¹ is COC₁-C₆ alkyl.

In some embodiments, R¹ is CO—C₆-C₁₀ aryl.

In some embodiments, R¹ is CO-5- to 10-membered heteroaryl.

In some embodiments, R¹ is CO₂C₁-C₆ alkyl.

In some embodiments, R¹ is CO₂C₃-C₈ cycloalkyl.

In some embodiments, R¹ is OCOC₁-C₆ alkyl.

In some embodiments, R¹ is OCOC₆-C₁₀ aryl.

In some embodiments, R¹ is OCO(5- to 10-membered heteroaryl).

In some embodiments, R¹ is OCO(3- to 7-membered heterocycloalkyl).

In some embodiments, R¹ is C₆-C₁₀ aryl.

In some embodiments, R¹ is phenyl.

In some embodiments, R¹ is 5- to 10-membered heteroaryl.

In some embodiments, R¹ is pyridyl (e.g., 4-pyridyl).

In some embodiments, R¹ is pyrazolyl (e.g., 1-pyrazolyl).

In some embodiments, R¹ is NH₂.

In some embodiments, R¹ is NHC₁-C₆ alkyl.

In some embodiments, R¹ is N(C₁-C₆ alkyl)₂.

In some embodiments, R¹ is CONR⁸R⁹.

In some embodiments, R¹ is SF₅.

In some embodiments, R¹ is SC₁-C₆ alkyl,

In some embodiments, R¹ is S(O₂)C₁-C₆ alkyl.

In some embodiments, R¹ is S(O₂)CH₃.

In some embodiments, R¹ is S(O₂)NR¹¹R¹²

In some embodiments, R¹ is S(O₂)N(CH₃)₂.

In some embodiments, R¹ is S(O)C₁-C₆ alkyl.

In some embodiments, R¹ is S(O)CH₃.

In some embodiments, R¹ is attached to a carbon of an aryl ring A.

In some embodiments, R¹ is attached to a carbon of a heteroaryl ring A.

In some embodiments, R¹ is attached to a nitrogen of a heteroaryl ring A.

### Particular Embodiments Wherein m=1 and n=1

In some embodiments, R¹ is C₁-C₆ alkyl optionally substituted with one or more hydroxy, and R² is C₁-C₆ alkyl optionally substituted with one or more hydroxy.

In some embodiments, R¹ is 1-hydroxy-2-methylpropan-2-yl, and R² is methyl.

In some embodiments, R¹ is 2-hydroxy-2-propyl and R² is methyl.

In some embodiments, R¹ is 2-hydroxy-2-propyl and R² is isopropyl.

In some embodiments, R¹ is 2-hydroxy-2-propyl and R² is 2-hydroxy-2-propyl.

In some embodiments, R¹ is 2-hydroxy-2-propyl and R² is 1-hydroxyethyl.

In some embodiments, R¹ is hydroxymethyl and R² is methyl.

In some embodiments, R¹ is hydroxymethyl and R² is ethyl.

In some embodiments, R¹ is 1-hydroxyethyl and R² is methyl.

In some embodiments, R¹ is 2-hydroxyethyl and R² is methyl.

In some embodiments, R¹ is 1-hydroxy-2-propyl and R² is methyl.

In some embodiments, R¹ is C₁-C₆ alkyl optionally substituted with one or more hydroxy, and R² is C₆-C₁₀ aryl.

In some embodiments, R¹ is 2-hydroxy-2-propyl and R² is phenyl.

In some embodiments, R¹ is C₁-C₆ alkyl optionally substituted with one or more hydroxy, and R² is 5- to 10-membered heteroaryl.

In some embodiments, R¹ is 2-hydroxy-2-propyl and R² is pyridyl.

In some embodiments, R¹ is 2-hydroxy-2-propyl and R² is pyrazolyl.

In some embodiments, R¹ is C₁-C₆ alkyl optionally substituted with one or more hydroxy, and R² is SF₅.

In some embodiments, R¹ is C₁-C₆ alkyl optionally substituted with one or more hydroxy, and R² is SC₁-C₆ alkyl.

In some embodiments, R¹ is C₁-C₆ alkyl optionally substituted with one or more hydroxy, and R² is S(O₂)C₁-C₆ alkyl.

In some embodiments, R¹ is C₁-C₆ alkyl optionally substituted with one or more hydroxy, and R² is S(O₂)CH₃.

In some embodiments, R¹ is C₁-C₆ alkyl optionally substituted with one or more hydroxy, and R² is halo.

In some embodiments, R¹ is 2-hydroxy-2-propyl and R² is chloro.

In some embodiments, R¹ is 2-hydroxy-2-propyl and R² is fluoro.

In some embodiments, R¹ is 1,2-dihydroxy-2-propyl and R² is fluoro.

In some embodiments, R¹ is 1,2-dihydroxy-2-propyl and R² is chloro.

In some embodiments, R¹ is C₁-C₆ alkyl optionally substituted with one or more hydroxy, and R² is C₁-C₆ alkyl optionally substituted with one or more C₁-C₆ alkoxy.

In some embodiments, R¹ is 2-hydroxy-2-propyl and R² is methoxymethyl.

In some embodiments, R¹ is C₁-C₆ alkyl optionally substituted with one or more hydroxy, and R² is C₆-C₁₀ aryl wherein the aryl is optionally substituted as defined elsewhere herein.

In some embodiments, R¹ is 2-hydroxy-2-propyl and R² is fluorophenyl.

In some embodiments, R¹ is C₁-C₆ alkyl optionally substituted with one or more hydroxy, and R² is C₆-C₁₀ aryl wherein the aryl is unsubstituted.

In some embodiments, R¹ is 2-hydroxy-2-propyl and R² is phenyl.

In some embodiments, R¹ is C₃-C₇ cycloalkyl optionally substituted with one or more hydroxy, and R² is C₁-C₆ alkyl.

In some embodiments, R¹ is 1-hydroxy-1-cyclopropyl, and R² is methyl.

In some embodiments, R¹ is 1-hydroxy-1-cyclobutyl, and R² is methyl.

In some embodiments, R¹ is 1-hydroxy-1-cyclopentyl, and R² is methyl.

In some embodiments, R¹ is 1-hydroxy-1-cyclohexyl, and R² is methyl.

In some embodiments, R¹ is 3- to 7-membered heterocycloalkyl optionally substituted with one or more hydroxy, and R² is C₁-C₆ alkyl.

In some embodiments, R¹ is morpholinyl, and R² is methyl.

In some embodiments, R¹ is 1,3-dioxolan-2-yl, and R² is methyl.

In some embodiments, R¹ is 3- to 7-membered heterocycloalkyl optionally substituted with one or more hydroxy, and R² is halo.

In some embodiments, R¹ is 1,3-dioxolan-2-yl, and R² is fluoro.

In some embodiments, R¹ is 1,3-dioxolan-2-yl, and R² is chloro.

In some embodiments, R¹ is C₁-C₆ alkyl optionally substituted with one or more oxo, and R² is methyl.

In some embodiments, R¹ is COCH₃, and R² is methyl.

In some embodiments, R¹ is C₁-C₆ alkyl optionally substituted with one or more C₁-C₆ alkoxy, and R² is C₁-C₆ alkyl.

In some embodiments, R¹ is 2-methoxy-2-propyl, and R² is methyl.

In some embodiments, R¹ is C₁-C₆ alkyl optionally substituted with one or more NR⁸R⁹, and R² is C₁-C₆ alkyl.

In some embodiments, R¹ is (dimethylamino)methyl, and R² is methyl.

In some embodiments, R¹ is C₁-C₆ alkyl optionally substituted with one or more NR⁸R⁹, and R² is halo.

In some embodiments, R¹ is C₁-C₆ alkyl substituted with one or more hydroxy; and R² is C₁-C₆ alkyl substituted with one or more hydroxy. In certain of the foregoing embodiments, R¹ or R² is further optionally substituted as defined elsewhere herein (e.g., R¹ or R² is further optionally substituted with one R⁵)

In some embodiments, R¹ is C₁-C₆ alkyl substituted with one or more hydroxy; and R² is hydroxymethyl.

In some embodiments, R¹ is 1,3-dihydroxy-2-methyl-2-propyl; and R² is hydroxymethyl.

In some embodiments, R¹ is 2-hydroxymethyl-2-propyl; and R² is hydroxymethyl.

In some embodiments, R¹ is 2-hydroxyeth-1-yl; and R² is hydroxymethyl.

In some embodiments, R¹ is 1,2-dihydroxy-3-propyl; and R² is hydroxymethyl.

In some embodiments, R¹ is 1,2,3-trihydroxy-2-propyl; and R² is hydroxymethyl.

In some embodiments, R¹ is 2-hydroxy-2-propyl; and R² is hydroxymethyl.

In some embodiments, R¹ is 1,2-dihydroxy-2-propyl; and R² is hydroxymethyl.

In some embodiments, R¹ is:

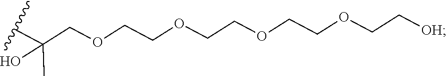 [SMILES: *C(C)(O)COCCOCCOCCOCCO]

and R² is hydroxymethyl.

In some embodiments, R² is C₁-C₆ alkyl substituted with one or more hydroxy; and R¹ is hydroxymethyl.

In some embodiments, R² is 1,3-dihydroxy-2-methyl-2-propyl; and R¹ is hydroxymethyl.

In some embodiments, R² is 2-hydroxymethyl-2-propyl; and R¹ is hydroxymethyl.

In some embodiments, R² is 2-hydroxyeth-1-yl; and R¹ is hydroxymethyl.

In some embodiments, R² is 1,2-dihydroxy-3-propyl; and R¹ is hydroxymethyl.

In some embodiments, R² is 1,2,3-trihydroxy-2-propyl; and R¹ is hydroxymethyl.

In some embodiments, R² is 2-hydroxy-2-propyl; and R¹ is hydroxymethyl.

In some embodiments, R² is 1,2-dihydroxy-2-propyl; and R¹ is hydroxymethyl.

In some embodiments, R² is:

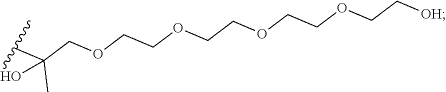 [SMILES: *C(C)(O)COCCOCCOCCOCCO]

and R¹ is hydroxymethyl.

In some embodiments, R² is 1-hydroxy-2-methylpropan-2-yl, and R¹ is methyl.

In some embodiments, R² is 2-hydroxy-2-propyl and R¹ is methyl.

In some embodiments, R² is 2-hydroxy-2-propyl and R¹ is isopropyl.

In some embodiments, R² is 2-hydroxy-2-propyl and R¹ is 1-hydroxyethyl.

In some embodiments, R² is hydroxymethyl and R¹ is methyl.

In some embodiments, R² is 1-hydroxyethyl and R¹ is methyl.

In some embodiments, R² is 2-hydroxyethyl and R¹ is methyl.

In some embodiments, R² is 1-hydroxy-2-propyl and R¹ is methyl.

In some embodiments, R² is C₁-C₆ alkyl optionally substituted with one or more hydroxy, and R¹ is C₆-C₁₀ aryl.

In some embodiments, R² is 2-hydroxy-2-propyl and R¹ is phenyl.

In some embodiments, R² is C₁-C₆ alkyl optionally substituted with one or more hydroxy, and R¹ is 5- to 10-membered heteroaryl.

In some embodiments, R² is 2-hydroxy-2-propyl and R¹ is pyridyl.

In some embodiments, R² is 2-hydroxy-2-propyl and R¹ is pyrazolyl.

In some embodiments, R² is C₁-C₆ alkyl optionally substituted with one or more hydroxy, and R¹ is SF₅.

In some embodiments, R² is C₁-C₆ alkyl optionally substituted with one or more hydroxy, and R¹ is SC₁-C₆ alkyl.

In some embodiments, R² is C₁-C₆ alkyl optionally substituted with one or more hydroxy, and R¹ is S(O₂)C₁-C₆ alkyl.

In some embodiments, R² is C₁-C₆ alkyl optionally substituted with one or more hydroxy, and R¹ is S(O₂)CH₃.

In some embodiments, R² is C₁-C₆ alkyl optionally substituted with one or more hydroxy, and R¹ is halo.

In some embodiments, R² is 2-hydroxy-2-propyl and R¹ is chloro.

In some embodiments, R² is 2-hydroxy-2-propyl and R¹ is fluoro.

In some embodiments, R² is 1,2-dihydroxy-2-propyl and R¹ is fluoro.

In some embodiments, R² is 1,2-dihydroxy-2-propyl and R¹ is chloro.

In some embodiments, R¹ is C₁-C₆ alkyl optionally substituted with one or more hydroxy, and R² is C₁-C₆ alkyl optionally substituted with one or more C₁-C₆ alkoxy.

In some embodiments, R¹ is 2-hydroxy-2-propyl and R² is methoxymethyl.

In some embodiments, R² is C₁-C₆ alkyl optionally substituted with one or more hydroxy, and R¹ is C₆-C₁₀ aryl wherein the aryl is optionally substituted as defined elsewhere herein.

In some embodiments, R² is 2-hydroxy-2-propyl and R¹ is fluorophenyl.

In some embodiments, R² is C₁-C₆ alkyl optionally substituted with one or more hydroxy, and R¹ is C₆-C₁₀ aryl wherein the aryl is unsubstituted.

In some embodiments, R² is 2-hydroxy-2-propyl and R¹ is phenyl.

In some embodiments, R² is C₃-C₇ cycloalkyl optionally substituted with one or more hydroxy, and R¹ is C₁-C₆ alkyl.

In some embodiments, R² is 1-hydroxy-1-cyclopropyl, and R¹ is methyl.

In some embodiments, R² is 1-hydroxy-1-cyclobutyl, and R¹ is methyl.

In some embodiments, R² is 1-hydroxy-1-cyclopentyl, and R¹ is methyl.

In some embodiments, R² is 1-hydroxy-1-cyclohexyl, and R¹ is methyl.

In some embodiments, R² is 3- to 7-membered heterocycloalkyl optionally substituted with one or more hydroxy, and R¹ is C₁-C₆ alkyl.

In some embodiments, R² is morpholinyl, and R¹ is methyl.

In some embodiments, R² is 1,3-dioxolan-2-yl, and R¹ is methyl.

In some embodiments, R² is 3- to 7-membered heterocycloalkyl optionally substituted with one or more hydroxy, and R¹ is halo.

In some embodiments, R² is 1,3-dioxolan-2-yl, and R¹ is fluoro.

In some embodiments, R² is 1,3-dioxolan-2-yl, and R¹ is chloro.

In some embodiments, R² is C₁-C₆ alkyl optionally substituted with one or more oxo, and R¹ is methyl.

In some embodiments, R² is COCH₃, and R¹ is methyl.

In some embodiments, R² is C₁-C₆ alkyl optionally substituted with one or more C₁-C₆ alkoxy, and R¹ is C₁-C₆ alkyl.

In some embodiments, R² is 2-methoxy-2-propyl, and R¹ is methyl.

In some embodiments, R² is C₁-C₆ alkyl optionally substituted with one or more NR⁸R⁹, and R¹ is C₁-C₆ alkyl.

In some embodiments, R² is (dimethylamino)methyl, and R¹ is methyl.

In some embodiments, R² is C₁-C₆ alkyl optionally substituted with one or more NR⁸R⁹, and R¹ is halo.

In some embodiments, R² is (dimethylamino)methyl, and R¹ is fluoro.

In some embodiments, R¹ and R² are each attached to a carbon of an aryl ring A.

In some embodiments, R¹ and R² are each attached to a carbon of a heteroaryl ring A.

In some embodiments, R¹ is attached to a carbon and R² is attached to a nitrogen of a heteroaryl ring A.

In some embodiments, R² is attached to a carbon and R¹ is attached to a nitrogen of a heteroaryl ring A.

In some embodiments, R¹ and R² are on adjacent atoms, and taken together with the atoms connecting them, form a C₅ carbocyclic ring optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R¹ and R² are on adjacent atoms, and taken together with the atoms connecting them, form a C₅ aliphatic carbocyclic ring.

In some embodiments, R¹ and R² are on adjacent atoms, and taken together with the atoms connecting them, form a C₅ saturated carbocyclic ring.

In some embodiments, R¹ and R² are on adjacent atoms, and taken together with the atoms connecting them, form a C₆ aromatic carbocyclic ring.

In some embodiments, R¹ and R² are on adjacent atoms, and taken together with the atoms connecting them, form a C₆ carbocyclic ring optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R¹ and R² are on adjacent atoms, and taken together with the atoms connecting them, form a C₆ aliphatic carbocyclic ring.

In some embodiments, R¹ and R² are on adjacent atoms, and taken together with the atoms connecting them, form a C₆ saturated carbocyclic ring.

In some embodiments, R¹ and R² are on adjacent atoms, and taken together with the atoms connecting them, form a C₆ aromatic carbocyclic ring.

In some embodiments, R¹ and R² are on adjacent atoms, and taken together with the atoms connecting them, form a 5-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R¹ and R² are on adjacent atoms, and taken together with the atoms connecting them, form a 5-membered aliphatic heterocyclic ring containing 1 or 2 heteroatoms independently selected from O, N, and S.

In some embodiments, R¹ and R² are on adjacent atoms, and taken together with the atoms connecting them, form a 5-membered heteroaromatic ring containing 1 or 2 heteroatoms independently selected from O, N, and S.

In some embodiments, R¹ and R² are on adjacent atoms, and taken together with the atoms connecting them, form a 6-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R¹ and R² are on adjacent atoms, and taken together with the atoms connecting them, form a 6-membered aliphatic heterocyclic ring containing 1 or 2 heteroatoms independently selected from O, N, and S.

In some embodiments, R¹ and R² are on adjacent atoms, and taken together with the atoms connecting them, form a 6-membered heteroaromatic ring containing 1 or 2 heteroatoms independently selected from O, N, and S.

In some embodiments, R¹ and R² are different.

In some embodiments, R¹ and R² are different, and R² comprises a carbonyl group.

In some embodiments, R¹ and R² are different, and R² comprises 1 or 2 (e.g., 1) nitrogen atoms.

In some embodiments, R¹ and R² are different, and R² comprises 1 or 2 (e.g., 1) oxygen atoms.

In some embodiments, R¹ and R² are different, and R² comprises a sulfur atom.

In some embodiments, R² and R¹ are different, and R² comprises a carbonyl group.

In some embodiments, R² and R¹ are different, and R² comprises 1 or 2 (e.g., 1) nitrogen atoms.

In some embodiments, R² and R¹ are different, and R² comprises 1 or 2 (e.g., 1) oxygen atoms.

In some embodiments, R² and R¹ are different, and R² comprises a sulfur atom.

In some embodiments, R¹ and R² are the same.

In some embodiments, R¹ is para or meta to R².

In some embodiments, R¹ is para or ortho to R².

In some embodiments, R¹ is ortho or meta to R².

In some embodiments, R¹ is para to R².

In some embodiments, R¹ is meta to R².

In some embodiments, R¹ is ortho to R².

### The Variables o and p

In some embodiments o=1 or 2.

In some embodiments o=1.

In some embodiments o=2.

In some embodiments p=0, 1, 2, or 3.

In some embodiments p=0.

In some embodiments p=1.

In some embodiments p=2.

In some embodiments, o=1 and p=0.

In some embodiments, o=2 and p=0.

In some embodiments, o=1 and p=1.

In some embodiments, o=1 and p=2.

In some embodiments, o=2 and p=1.

In some embodiments, o=2 and p=2.

In some embodiments, o=2 and p=3.

### The Ring B and Substitutions on the Ring B

In some embodiments, B is a 5-10-membered monocyclic or bicyclic heteroaryl or a C₆-C₁₀ monocyclic or bicyclic aryl, such as phenyl.

In some embodiments, B is a 5-6-membered monocyclic heteroaryl or a C₆ monocyclic aryl.

In some embodiments, B is a 5-10-membered monocyclic or bicyclic heteroaryl.

In some embodiments, B is a C₆-C₁₀ monocyclic or bicyclic aryl.

In some embodiments, B is a 5-membered monocyclic or bicyclic heteroaryl.

In some embodiments, B is a 7-10 membered monocyclic or bicyclic heteroaryl.

In some embodiments, B is a 6-membered bicyclic heteroaryl.

In some embodiments, B is a 6-membered monocyclic heteroaryl containing 2 or more N atoms.

In some embodiments, B is phenyl, o is 1 or 2, and p is 0, 1, 2, or 3.

In some embodiments, B is pyridyl, o is 1 or 2, and p is 0, 1, 2, or 3.

In some embodiments, B is 3-pyridyl, o is 1 or 2, and p is 0, 1, 2, or 3.

In some embodiments, B is phenyl, o is 1, or 2, and p is 0, 1, 2, or 3.

In some embodiments, B is pyridyl (e.g., 3-pyridyl), o is 1 or 2, and p is 0, 1, 2, or 3.

In some embodiments, B is phenyl, o is 1, and p is 1, 2, or 3.

In some embodiments, B is phenyl, o is 2, and p is 1, 2, or 3.

In some embodiments, B is pyridyl (e.g., 3-pyridyl), o is 1, and p is 0, 1, 2, or 3.

In some embodiments, B is pyridyl (e.g., 3-pyridyl), o is 2, and p is 0, 1, 2, or 3.

In some embodiments, B is phenyl, o is 1 or 2, and p is 0, 1, 2, or 3.

In some embodiments, B is pyridyl (e.g., 3-pyridyl), o is 1 or 2, and p is 0, 1, 2, or 3.

In some embodiments, B is phenyl, o is 1, and p is 0, 1, 2, or 3.

In some embodiments, B is phenyl, o is 2, and p is 0, 1, 2, or 3.

In some embodiments, B is pyridyl (e.g., 3-pyridyl), o is 1, and p is 0, 1, 2, or 3.

In some embodiments, B is pyridyl (e.g., 3-pyridyl), o is 2, and p is 0, 1, or 2.

In some embodiments, B is pyrimidinyl (e.g., pyrimidin-5-yl), o is 1, and p is 0, 1, or 2.

In some embodiments, B is pyrimidinyl (e.g., pyrimidin-5-yl), o is 2, and p is 0 or 1.

In some embodiments, B is one of the rings disclosed hereinbelow, substituted as disclosed hereinbelow, wherein in each case the bond that is shown as being broken by the wavy line [symbol] connects B to the NH(CO) group of Formula AA.

In some embodiments, the substituted ring B

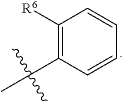

In some embodiments, the substituted ring B is

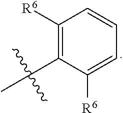

In some embodiments, the substituted ring B is

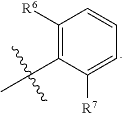

In some embodiments, the substituted ring B is

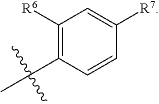

In some embodiments, the substituted ring B is

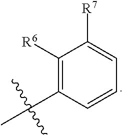

In some embodiments, the substituted ring B is

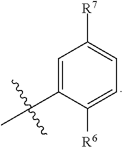

In some embodiments, the substituted ring B is

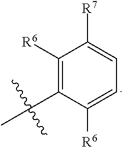

In some embodiments, the substituted ring B is

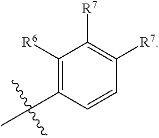

In some embodiments, the substituted ring B is

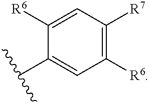

In some embodiments, the substituted ring B is

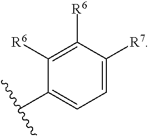

In some embodiments, the substituted ring B is

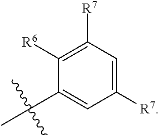

In some embodiments, the substituted ring B is

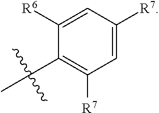

In some embodiments, the substituted ring B is

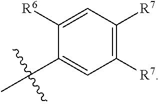

In some embodiments, the substituted ring B is

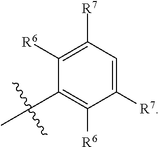

In some embodiments, the substituted ring B is

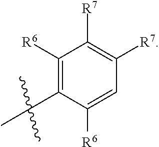

In some embodiments, the substituted ring B is

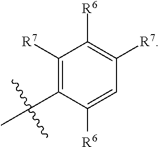

In some embodiments, the substituted ring B is

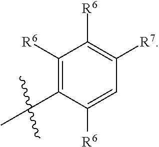

In some embodiments, the substituted ring B is

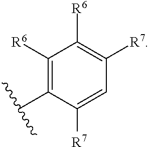

In some embodiments, the substituted ring B is

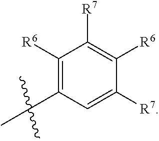

In some embodiments, the substituted ring B is

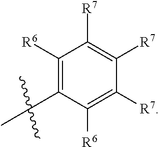

In some embodiments, the substituted ring B is

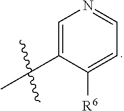

In some embodiments, the substituted ring B is

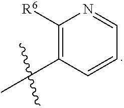

In some embodiments, the substituted ring B is

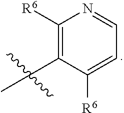

In some embodiments, the substituted ring B is

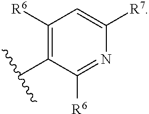

In some embodiments, the substituted ring B is

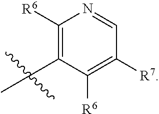

In some embodiments, the substituted ring B is

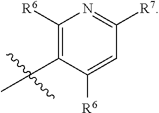

In some embodiments, the substituted ring B is

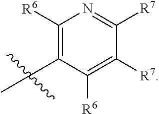

In some embodiments, the substituted ring B is

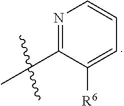

In some embodiments, the substituted ring B is

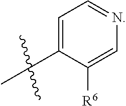

In some embodiments, the substituted ring B is

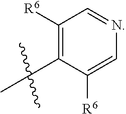

In some embodiments, the substituted ring B is

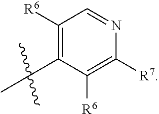

In some embodiments, the substituted ring B is

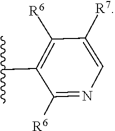

In some embodiments, the substituted ring B is

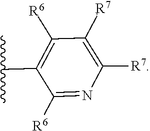

In some embodiments, the substituted ring B is

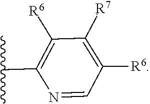

In some embodiments, the substituted ring B is

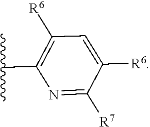

In some embodiments, the substituted ring B is

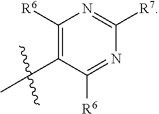

In some embodiments, the substituted B ring is selected from the group consisting of (B-1) to (B-15).

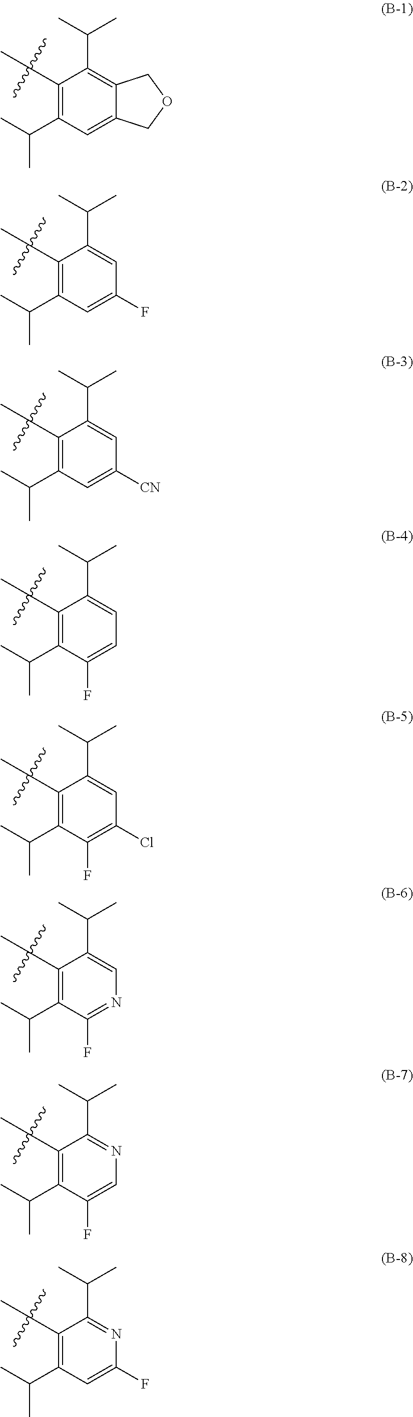

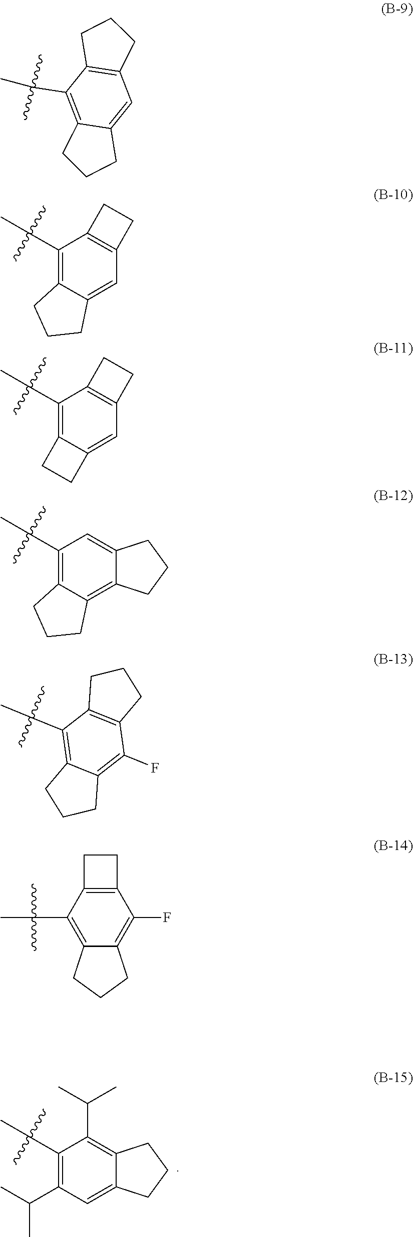

The Groups R⁶ and R⁷

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹′ wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and a C₂-C₆ alkenyl,
wherein R⁶ and R⁷ are each optionally substituted with one or more substituents independently selected from
hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl,
C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₆ aliphatic carbocyclic ring or at least one 5- to 6-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₅-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein the C₃-C₇ cycloalkyl, C₁-C₆ haloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₅-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, halo, CN, NO₂, COC₁-C₆ alkyl, CO₂C₁-C₆ alkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein the C₁-C₆ alkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₅-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, halo, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹, wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₅-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, halo, CN, NO₂, COC₁-C₆ alkyl, CO₂C₁-C₆ alkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein the C₁-C₆ alkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₅-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein the C₃-C₇ cycloalkyl, C₁-C₆ haloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₅-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, halo, CN, NO₂, COC₁-C₆ alkyl, CO₂C₁-C₆ alkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein the C₁-C₆ alkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₅-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are unsubstituted;
   or at least one pair of R⁶ and R⁷ on adjacent atoms, taken together with the atoms connecting them, independently form at least one C₅-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein the C₁-C₆ alkyl, C₃-C₇ cycloalkyl and 3- to 7-membered heterocycloalkyl are each unsubstituted;
or at least one pair of R⁶ and R⁷ on adjacent atoms, taken together with the atoms connecting them, independently form at least one C₅-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
and R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo,
or at least one pair of R⁶ and R⁷ on adjacent atoms, taken together with the atoms connecting them, independently form at least one C₅-C₈ carbocyclic ring, wherein the carbocyclic ring is optionally independently substituted with one or more hydroxy or oxo.

In some embodiments,

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo,
or at least one pair of R⁶ and R⁷ on adjacent atoms, taken together with the atoms connecting them, independently form at least one C₄-C₆ aliphatic carbocyclic ring, wherein the carbocyclic ring is optionally independently substituted with one or more hydroxy or oxo.

In some embodiments,

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo,
or at least one pair of R⁶ and R⁷ on adjacent atoms, taken together with the atoms connecting them, independently form at least one 5- to 8-membered heterocyclic ring containing 1 or 2 heteroatoms independently selected from O, N, and S, wherein the heterocyclic ring is optionally independently substituted with one or more hydroxy or oxo.

In some embodiments,

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo,
or at least one pair of R⁶ and R⁷ on adjacent atoms, taken together with the atoms connecting them, independently form at least one C₄-C₈ carbocyclic ring, wherein the carbocyclic ring is optionally independently substituted with one or more hydroxy or oxo.

In some embodiments,

at least one pair of R⁶ and R⁷ on adjacent atoms, taken together with the atoms connecting them, independently form at least one C₄-C₆ aliphatic carbocyclic ring, wherein the carbocyclic ring is optionally independently substituted with one or more hydroxy or oxo.

In some embodiments,

at least one pair of R⁶ and R⁷ on adjacent atoms, taken together with the atoms connecting them, independently form at least one C₄ aliphatic carbocyclic ring, wherein the carbocyclic ring is optionally independently substituted with one or more hydroxy or oxo.

In some embodiments,

at least one pair of R⁶ and R⁷ on adjacent atoms, taken together with the atoms connecting them, independently form at least one C₅ aliphatic carbocyclic ring, wherein the carbocyclic ring is optionally independently substituted with one or more hydroxy or oxo.

In some embodiments,

at least one pair of R⁶ and R⁷ on adjacent atoms, taken together with the atoms connecting them, independently form at least one C₆ aliphatic carbocyclic ring, wherein the carbocyclic ring is optionally independently substituted with one or more hydroxy or oxo.

In some embodiments,

at least one pair of R⁶ and R⁷ on adjacent atoms, taken together with the atoms connecting them, independently form at least one 5- to 6-membered heterocyclic ring containing 1 heteroatom independently selected from O, N, and S, wherein the heterocyclic ring is optionally independently substituted with one or more hydroxy or oxo.

In some embodiments,

at least one pair of R⁶ and R⁷ on adjacent atoms, taken together with the atoms connecting them, independently form at least one 5-membered heterocyclic ring containing 1 heteroatom independently selected from O, N, and S, wherein the heterocyclic ring is optionally independently substituted with one or more hydroxy or oxo.

In some embodiments,

at least one pair of R⁶ and R⁷ on adjacent atoms, taken together with the atoms connecting them, independently form at least one 6-membered heterocyclic ring containing 1 heteroatom independently selected from O, N, and S, wherein the heterocyclic ring is optionally independently substituted with one or more hydroxy or oxo.

In some embodiments,

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo,
or at least one pair of R⁶ and R⁷ on adjacent atoms, taken together with the atoms connecting them, independently form at least one C₄-C₆ aliphatic carbocyclic ring, wherein the carbocyclic ring is optionally independently substituted with one or more C₁-C₆ alkyl.

In some embodiments,

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo,
or at least one pair of R⁶ and R⁷ on adjacent atoms, taken together with the atoms connecting them, independently form at least one 5- to 8-membered heterocyclic ring containing 1 or 2 heteroatoms independently selected from O, N, and S, wherein the heterocyclic ring is optionally independently substituted with one or more C₁-C₆ alkyl.

In some embodiments,

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from C₁-C₆ alkyl.
or at least one pair of R⁶ and R⁷ on adjacent atoms, taken together with the atoms connecting them, independently form at least one C₄-C₈ carbocyclic ring, wherein the carbocyclic ring is optionally independently substituted with one or more C₁-C₆ alkyl.

In some embodiments,

at least one pair of R⁶ and R⁷ on adjacent atoms, taken together with the atoms connecting them, independently form at least one C₄-C₆ aliphatic carbocyclic ring, wherein the carbocyclic ring is optionally independently substituted with one or more C₁-C₆ alkyl.

In some embodiments,

at least one pair of R⁶ and R⁷ on adjacent atoms, taken together with the atoms connecting them, independently form at least one C₄ aliphatic carbocyclic ring, wherein the carbocyclic ring is optionally independently substituted with one or more C₁-C₆ alkyl.

In some embodiments,

at least one pair of R⁶ and R⁷ on adjacent atoms, taken together with the atoms connecting them, independently form at least one C₅ aliphatic carbocyclic ring, wherein the carbocyclic ring is optionally independently substituted with one or more C₁-C₆ alkyl.

In some embodiments,

at least one pair of R⁶ and R⁷ on adjacent atoms, taken together with the atoms connecting them, independently form at least one C₆ aliphatic carbocyclic ring, wherein the carbocyclic ring is optionally independently substituted with one or more C₁-C₆ alkyl.

In some embodiments,

at least one pair of R⁶ and R⁷ on adjacent atoms, taken together with the atoms connecting them, independently form at least one 5- to 6-membered heterocyclic ring containing 1 heteroatom independently selected from O, N, and S, wherein the heterocyclic ring is optionally independently substituted with one or more C₁-C₆ alkyl.

In some embodiments,

at least one pair of R⁶ and R⁷ on adjacent atoms, taken together with the atoms connecting them, independently form at least one 5-membered heterocyclic ring containing 1 heteroatom independently selected from O, N, and S, wherein the heterocyclic ring is optionally independently substituted with one or more C₁-C₆ alkyl.

In some embodiments,

at least one pair of R⁶ and R⁷ on adjacent atoms, taken together with the atoms connecting them, independently form at least one 6-membered heterocyclic ring containing 1 heteroatom independently selected from O, N, and S, wherein the heterocyclic ring is optionally independently substituted with one or more C₁-C₆ alkyl.

In some embodiments, o=1; p=0; and

R⁶ is selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, o=1; p=1; and

R⁶ is selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl, wherein the C₁-C₆ alkyl and 3- to 7-membered heterocycloalkyl is optionally substituted with one or more substituents each independently selected from hydroxy or oxo,
or at least one pair of R⁶ and R⁷ on adjacent atoms, taken together with the atoms connecting them, independently form at least one C₅-C₈ carbocyclic ring, wherein the carbocyclic ring is optionally independently substituted with one or more hydroxy or oxo.

In some embodiments, o=1 or 2; p=1, 2, or 3; and

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, o=2; p=1; and

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
and R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or at least one pair of R⁶ and R⁷ on adjacent atoms, taken together with the atoms connecting them, independently form at least one C₄-C₇ (e.g., C₄-C₆) carbocyclic ring (e.g., aliphatic carbocyclic ring) or at least one 5-to-7-membered (e.g., 5-to-6-membered) heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, o=1 or 2; p=1, 2, or 3; and

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo,
or at least one pair of R⁶ and R⁷ on adjacent atoms, taken together with the atoms connecting them, independently form at least one C₅-C₈ carbocyclic ring, wherein the carbocyclic ring is optionally independently substituted with one or more hydroxy or oxo.

In some embodiments, o=1 or 2; p=1, 2, or 3; and

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo.

In some embodiments, o=1 or 2; p=1, 2, or 3; and

one R⁶ and one R⁷ are on adjacent atoms, and taken together with the atoms connecting them, form a C₅-C₈ carbocyclic ring or a 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=1 or 2; p=1, 2, or 3; and

one R⁶ and one R⁷ are on adjacent atoms, and taken together with the atoms connecting them, form a C₆ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=1 or 2; p=1, 2, or 3; and

one R⁶ and one R⁷ are on adjacent atoms, and taken together with the atoms connecting them, form a C₅-C₈ carbocyclic ring or a 5-to-8-membered heterocyclic ring containing 1 or 2 heteroatoms independently selected from O, N, and S, wherein the carbocyclic ring or heterocyclic ring is unsubstituted.

In some embodiments, o=2; p=2 or 3; and

two pairs, each of one R⁶ and one R⁷, are on adjacent atoms, and each pair of one R⁶ and one R⁷ taken together with the atoms connecting them independently form a C₅-C₈ carbocyclic ring or a 5-to-8-membered heterocyclic ring containing 1 or 2 heteroatoms independently selected from O, N, and S, wherein each carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them independently form a C₆ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them independently form a C₅-C₈ carbocyclic ring or a 5-to-8-membered heterocyclic ring containing 1 or 2 heteroatoms independently selected from O, N, and S, wherein the carbocyclic ring or heterocyclic ring is unsubstituted.

### Particular Embodiments Wherein o=1; p=0

In some embodiments, R⁶ is C₁-C₆ alkyl.

In some embodiments, R⁶ is isopropyl.

In some embodiments, R⁶ is ethyl.

In some embodiments, R⁶ is methyl.

In some embodiments, R⁶ is C₁-C₆ alkyl substituted with one or more halo.

In some embodiments, R⁶ is C₁-C₆ alkyl (e.g., methyl) substituted with one or more (e.g., one) C₁-C₆ alkoxy (e.g., methoxy). In some embodiments, R⁶ is methoxymethyl.

In some embodiments, R⁶ is trifluoromethyl.

In some embodiments, R⁶ is trifluoromethoxy.

In some embodiments, R⁶ is C₃-C₇ cycloalkyl.

In some embodiments, R⁶ is cyclopropyl.

In some embodiments, R⁶ is halo.

In some embodiments, R⁶ is chloro.

In some embodiments, R⁶ is fluoro.

In some embodiments, R⁶ is cyano.

In some embodiments, R⁶ is attached to a carbon of an aryl ring B.

In some embodiments, R⁶ is attached to a carbon of a heteroaryl ring B.

In some embodiments, R⁶ is attached to a nitrogen of a heteroaryl ring B.

### Particular Embodiments Wherein o=1 or 2: p=1, 2, or 3

In some embodiments, at least one R⁶ is C₁-C₆ alkyl, and at least one R⁷ is C₁-C₆ alkyl optionally substituted with one or more halo.

In some embodiments, at least one R⁶ is C₁-C₆ alkyl, and at least one R⁷ is halo.

In some embodiments, at least one R⁶ is C₁-C₆ alkyl and at least one R⁷ is C₁-C₆ alkyl.

In some embodiments, at least one R⁶ is isopropyl and at least one R⁷ is methyl.

In some embodiments, at least one R⁶ is isopropyl and at least one R⁷ is isopropyl.

In some embodiments, o=1; p=1; R⁶ is isopropyl; and R⁷ is isopropyl.

In some embodiments, at least one R⁶ is C₁-C₆ alkyl, and at least one R⁷ is C₁-C₆ alkyl substituted with one or more halo.

In some embodiments, at least one R⁶ is isopropyl and at least one R⁷ is trifluoromethyl.

In some embodiments, at least one R⁶ is C₁-C₆ alkyl, and at least one R⁷ is C₃-C₇ cycloalkyl.

In some embodiments, at least one R⁶ is isopropyl and at least one R⁷ is cyclopropyl.

In some embodiments, o=1; p=1; R⁶ is isopropyl; and R⁷ is cyclopropyl.

In some embodiments, o=1; p=2; R⁶ is isopropyl; one R⁷ is cyclopropyl; and the other R⁷ is fluoro.

In some embodiments, o=2; p=2; one R⁶ is isopropyl; one R⁷ is cyclopropyl; the other R⁶ is cyano; and the other R⁷ is fluoro.

In some embodiments, at least one R⁶ is C₁-C₆ alkyl, and at least one R⁷ is halo.

In some embodiments, at least one R⁶ is isopropyl and at least one R⁷ is halo.

In some embodiments, at least one R⁶ is isopropyl and at least one R⁷ is chloro.

In some embodiments, at least one R⁶ is isopropyl and at least one R⁷ is fluoro.

In some embodiments, o=1; p=1; R⁶ is isopropyl; and R⁷ is chloro.

In some embodiments, o=2; p=1; at least one R⁶ is isopropyl; and R⁷ is chloro.

In some embodiments, o=1; p=1; R⁶ is isopropyl; and R⁷ is fluoro.

In some embodiments, o=2; p=1; at least one R⁶ is isopropyl, and R⁷ is fluoro.

In some embodiments, o=2; p=1; each R⁶ is isopropyl, and R⁷ is fluoro.

In some embodiments, o=1; p=1; R⁶ is isopropyl; and R⁷ is fluoro.

In some embodiments, o=2; p=1; at least one R⁶ is isopropyl; and R⁷ is fluoro.

In some embodiments, o=2; p=2; at least one R⁶ is isopropyl, one R⁷ is fluoro, and one R⁷ is phenyl substituted with trifluoromethyl (e.g., substituted at the meta position with trifluoromethyl).

In some embodiments, o=2; p=2; at least one R⁶ is isopropyl; and R⁷ is fluoro.

In some embodiments, o=2; p=2; at least one R⁶ is isopropyl; one R⁷ is fluoro; and the other R⁷ is cyano.

In some embodiments, o=2; p=3; at least one R⁶ is isopropyl; two R⁷ are fluoro; and one R⁷ is chloro.

In some embodiments, o=2; p=1; at least one R⁶ is ethyl; and R⁷ is fluoro.

In some embodiments, o=2; p=1; at least one R⁶ is isopropyl; the other R⁶ is trifluoromethyl; and R⁷ is chloro.

In some embodiments, at least one R⁶ is C₁-C₆ alkyl, and at least one R⁷ is cyano.

In some embodiments, at least one R⁶ is isopropyl and at least one R⁷ is cyano.

In some embodiments, o=1; p=1; R⁶ is isopropyl; and R⁷ is cyano.

In some embodiments, o=2; p=1; at least one R⁶ is isopropyl; and R⁷ is cyano.

In some embodiments, at least one R⁶ is C₃-C₇ cycloalkyl, and at least one R⁷ is C₃-C₇ cycloalkyl.

In some embodiments, at least one R⁶ is cyclopropyl, and at least one R⁷ is cyclopropyl.

In some embodiments, at least one R⁶ is C₃-C₇ cycloalkyl, and at least one R⁷ is halo.

In some embodiments, at least one R⁶ is cyclopropyl and at least one R⁷ is halo.

In some embodiments, at least one R⁶ is cyclopropyl and at least one R⁷ is chloro.

In some embodiments, at least one R⁶ is cyclopropyl and at least one R⁷ is fluoro.

In some embodiments, o=1; p=1; R⁶ is cyclopropyl; and R⁷ is chloro.

In some embodiments, o=1; p=1; R⁶ is cyclopropyl; and R⁷ is fluoro.

In some embodiments, at least one R⁶ is C₁-C₆ alkyl, and at least one R⁷ is C₁-C₆ alkoxy optionally substituted with one or more halo.

In some embodiments, at least one R⁶ is isopropyl, and at least one R⁷ is C₁-C₆ alkoxy.

In some embodiments, at least one R⁶ is isopropyl, and at least one R⁷ is methoxy.

In some embodiments, o=1; p=1; R⁶ is isopropyl, and R⁷ is methoxy.

In some embodiments, o=2; p=1; at least one R⁶ is isopropyl, and R⁷ is methoxy.

In some embodiments, at least one R⁶ is C₁-C₆ alkyl, and at least one R⁷ is C₁-C₆ alkoxy substituted with one or more halo.

In some embodiments, at least one R⁶ is isopropyl, and at least one R⁷ is trifluoromethoxy.

In some embodiments, at least one R⁶ is isopropyl, and at least one R⁷ is difluoromethoxy.

In some embodiments, at least one R⁶ is C₁-C₆ alkyl, and at least one R⁷ is C₁-C₆ alkyl (e.g., methyl) substituted with one or more (e.g., one) C₁-C₆ alkoxy (e.g., methoxy).

In some embodiments, at least on R⁶ is isopropyl, and at least one R⁷ is methoxymethyl.

In some embodiments, o=2; p=1, each R⁶ is isopropyl, and R⁷ is methoxymethyl.

In some embodiments, at least one R⁶ is halo, and at least one R⁷ is C₁-C₆ haloalkyl optionally substituted with hydroxy.

In some embodiments, o=1; p=1; R⁶ is chloro, and R⁷ is trifluoromethyl.

In some embodiments, at least one R⁶ is halo, and at least one R⁷ is C₁-C₆ haloalkoxy.

In some embodiments, at least one R⁶ is chloro, and at least one R⁷ is trifluoromethoxy.

In some embodiments, o=1; p=1; R⁶ is chloro, and R⁷ is trifluoromethoxy.

In some embodiments, at least one R⁶ is C₁-C₆ alkoxy; and at least one R⁷ is halo.

In some embodiments, o=1; p=2; R⁶ is C₁-C₆ alkoxy; and at least one R⁷ is chloro.

In some embodiments, at least one R⁶ is C₃-C₇ cycloalkyl; and at least one R⁷ is C₁-C₆ haloalkyl optionally substituted with hydroxy.

In some embodiments, at least one R⁶ is cyclopropyl; and at least one R⁷ is trifluoromethyl.

In some embodiments, o=1; p=2; R⁶ is cyclopropyl; one R⁷ is trifluoromethyl; and the other R⁷ is fluoro.

In some embodiments, at least one R⁶ is C₁-C₆ alkyl, and at least one R⁷ is C₆-C₁₀ aryl, wherein the C₆-C₁₀ aryl is optionally substituted or optionally fused as described elsewhere herein.

In some embodiments, at least one R⁶ is isopropyl, and at least one R⁷ is C₆-C₁₀ aryl, wherein the C₆-C₁₀ aryl is optionally substituted as described elsewhere herein.

In some embodiments, at least one R⁶ is isopropyl, and at least one R⁷ is dichlorophenyl (e.g., 3,4-dichlorophenyl).

In some embodiments, at least one R⁶ is isopropyl, and at least one R⁷ is dimethylphenyl (e.g., 3,4-dimethylphenyl).

In some embodiments, at least one R⁶ is isopropyl, and at least one R⁷ is naphthyl (e.g., napthyl substituted with one methoxy).

In some embodiments, at least one R⁶ is isopropyl, and at least one R⁷ is C₆-C₁₀ aryl, wherein the C₆-C₁₀ aryl is optionally fused to a five-to-seven-membered carbocyclic ring or heterocyclic ring containing one or two heteroatoms independently selected from oxygen, sulfur and nitrogen (e.g., R⁷ is

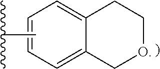

In some embodiments, o=2; p=1, each R⁶ is isopropyl; and R⁷ is C₆-C₁₀ aryl, wherein the C₆-C₁₀ aryl is optionally substituted as described elsewhere herein (e.g., R⁷ is dimethylphenyl; or R⁷ is dichlorophenyl; or R⁷ is naphthyl).

In some embodiments, o=2; p=1, each R⁶ is isopropyl; and R⁷ is C₆-C₁₀ aryl, wherein the C₆-C₁₀ aryl is optionally fused to a five-to-seven-membered carbocyclic ring or heterocyclic ring containing one or two heteroatoms independently selected from oxygen, sulfur and nitrogen (e.g., R⁷ is

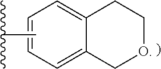

In some embodiments, at least one R⁶ is C₁-C₆ alkyl, and at least one R⁷ is 3- to 7-membered heterocycloalkyl, wherein the 3- to 7-membered heterocycloalkyl is optionally substituted or optionally fused as described elsewhere herein.

In some embodiments, at least one R⁶ is isopropyl, and at least one R⁷ is 3- to 7-membered heterocycloalkyl, wherein the 3- to 7-membered heterocycloalkyl is optionally substituted as described elsewhere herein.

In some embodiments, at least one R⁶ is isopropyl, and at least one R⁷ is tetrahydrofuranyl.

In some embodiments, at least one R⁶ is isopropyl, and at least one R⁷ is

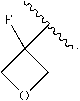 [SMILES: *C1(F)COC1]

In some embodiments, at least one R⁶ is C₁-C₆ alkyl, and at least one R⁷ is 5- to 10-membered heteroaryl, wherein the 5- to 10-membered heteroaryl is optionally substituted or optionally fused as described elsewhere herein.

In some embodiments, at least one R⁶ isopropyl, and at least one R⁷ is 5- to 10-membered heteroaryl, wherein the 5- to 10-membered heteroaryl is optionally substituted or optionally fused as described elsewhere herein.

In some embodiments, at least one R⁶ isopropyl, and at least one R⁷ is pyrazolyl.

In some embodiments, at least one R⁶ is C₁-C₆ alkyl, and at least one R⁷ is C₁-C₆ alkyl substituted with C₃-C₇ cycloalkyl.

In some embodiments, at least one R⁶ is isopropyl, and at least one R⁷ is ethyl substituted with cyclohexyl.

In some embodiments, at least one R⁶ is C₁-C₆ alkyl, and at least one R⁷ is C₁-C₆ alkyl substituted with C₃-C₇ cycloalkyl and oxo.

In some embodiments, at least one R⁶ is isopropyl, and at least one R⁷ is ethyl substituted with cyclohexyl and oxo.

In some embodiments, at least one R⁶ is C₁-C₆ alkyl, and at least one R⁷ is C₁-C₆ alkyl substituted with 4-6 membered heterocycloalkyl (e.g., tetrahydropyranyl).

In some embodiments, at least one R⁶ is C₁-C₆ alkyl, and at least one R⁷ is C₁-C₆ alkyl substituted with C₃-C₇ cycloalkoxy.

In some embodiments, at least one R⁶ is isopropyl, and at least one R⁷ is ethyl substituted with cyclopentoxy.

In some embodiments, at least one R⁶ is C₁-C₆ alkyl, and at least one R⁷ is C₂-C₆ (e.g., C₂) alkynyl substituted with C₃-C₇ cycloalkyl.

In some embodiments, at least one R⁶ is isopropyl, and at least one R⁷ is C₂ alkynyl substituted with cyclohexyl or cyclopentyl.

In some embodiments, at least one R⁶ is C₁-C₆ alkyl, and at least one R⁷ is C₂-C₆ (e.g., C₂) alkynyl substituted with 4-6 membered heterocycloalkyl.

In some embodiments, at least one R⁶ is isopropyl, and at least one R⁷ is C₂ alkynyl substituted with tetrahydropyranyl.

In some embodiments, at least one R⁷ is C₁-C₆ alkyl, and at least one R⁶ is C₁-C₆ alkyl optionally substituted with one or more halo.

In some embodiments, at least one R⁷ is isopropyl and at least one R⁶ is methyl.

In some embodiments, at least one R⁷ is C₁-C₆ alkyl, and at least one R⁶ is C₁-C₆ alkyl substituted with one or more halo.

In some embodiments, at least one R⁷ is isopropyl and at least one R⁶ is trifluoromethyl.

In some embodiments, at least one R⁷ is C₁-C₆ alkyl, and at least one R⁶ is C₃-C₇ cycloalkyl.

In some embodiments, at least one R⁷ is isopropyl and at least one R⁶ is cyclopropyl.

In some embodiments, o=1; p=1; R⁷ is isopropyl; and R⁶ is cyclopropyl.

In some embodiments, o=2; p=1; R⁷ is isopropyl; one R⁶ is cyclopropyl; and the other R⁶ is fluoro.

In some embodiments, o=2; p=2; one R⁷ is isopropyl; one R⁶ is cyclopropyl; the other R⁷ is cyano; and the other R⁶ is fluoro.

In some embodiments, at least one R⁷ is C₁-C₆ alkyl, and at least one R⁶ is halo.

In some embodiments, at least one R⁷ is isopropyl and at least one R⁶ is halo.

In some embodiments, at least one R⁷ is isopropyl and at least one R⁶ is chloro.

In some embodiments, at least one R⁷ is isopropyl and at least one R⁶ is fluoro.

In some embodiments, o=1; p=1; R⁷ is isopropyl; and R⁶ is chloro.

In some embodiments, o=2; p=1; at least one R⁷ is isopropyl; and at least one R⁶ is chloro.

In some embodiments, o=1; p=1; R⁷ is isopropyl; and R⁶ is fluoro.

In some embodiments, o=2; p=1; R⁷ is isopropyl; and at least one R⁶ is fluoro.

In some embodiments, o=1; p=2; each R⁷ is isopropyl, and R⁶ is fluoro.

In some embodiments, o=2; p=2; at least one R⁷ is isopropyl; and at least one R⁶ is fluoro.

In some embodiments, o=2; p=2; at least one R⁷ is isopropyl; one R⁶ is fluoro; and the other R⁶ is cyano.

In some embodiments, o=2; p=1; R⁷ is ethyl; and at least one R⁶ is fluoro.

In some embodiments, o=1; p=2; one R⁷ is isopropyl; the other R⁷ is trifluoromethyl; and R⁶ is chloro.

In some embodiments, at least one R⁷ is C₁-C₆ alkyl, and at least one R⁶ is cyano.

In some embodiments, at least one R⁷ is isopropyl and at least one R⁶ is cyano.

In some embodiments, o=1; p=1; R⁷ is isopropyl; and R⁶ is cyano.

In some embodiments, o=2; p=1; R⁷ is isopropyl; and at least one R⁶ is cyano.

In some embodiments, at least one R⁷ is C₃-C₇ cycloalkyl, and at least one R⁶ is C₃-C₇ cycloalkyl.

In some embodiments, at least one R⁷ is cyclopropyl, and at least one R⁶ is cyclopropyl.

In some embodiments, at least one R⁷ is C₃-C₇ cycloalkyl, and at least one R⁶ is halo.

In some embodiments, at least one R⁷ is cyclopropyl and at least one R⁶ is halo.

In some embodiments, at least one R⁷ is cyclopropyl and at least one R⁶ is chloro.

In some embodiments, at least one R⁷ is cyclopropyl and at least one R⁶ is fluoro.

In some embodiments, o=1; p=1; R⁷ is cyclopropyl; and R⁶ is chloro.

In some embodiments, o=1; p=1; R⁷ is cyclopropyl; and R⁶ is fluoro.

In some embodiments, at least one R⁷ is C₁-C₆ alkyl, and at least one R⁶ is C₁-C₆ alkoxy optionally substituted with one or more halo.

In some embodiments, at least one R⁷ is isopropyl, and at least one R⁶ is C₁-C₆ alkoxy.

In some embodiments, at least one R⁷ is isopropyl, and at least one R⁶ is methoxy.

In some embodiments, o=1; p=1; R⁷ is isopropyl, and R⁶ is methoxy.

In some embodiments, o=2; p=1; R⁷ is isopropyl, and at least one R⁶ is methoxy.

In some embodiments, at least one R⁷ is C₁-C₆ alkyl, and at least one R⁶ is C₁-C₆ alkoxy substituted with one or more halo.

In some embodiments, at least one R⁷ is isopropyl, and at least one R⁶ is trifluoromethoxy.

In some embodiments, at least one R⁷ is C₁-C₆ alkyl, and at least one R⁶ is C₁-C₆ alkyl (e.g., methyl) substituted with one or more (e.g., one) C₁-C₆ alkoxy (e.g., methoxy).

In some embodiments, at least on R⁷ is isopropyl, and at least one R⁶ is methoxymethyl.

In some embodiments, o=2; p=1, each R⁷ is isopropyl, and R⁶ is methoxymethyl.

In some embodiments, at least one R⁷ is halo, and at least one R⁶ is C₁-C₆ haloalkyl optionally substituted with hydroxy.

In some embodiments, o=1; p=1; R⁷ is chloro, and R⁶ is trifluoromethyl.

In some embodiments, at least one R⁷ is halo, and at least one R⁶ is C₁-C₆ haloalkoxy.

In some embodiments, at least one R⁷ is chloro, and at least one R⁶ is trifluoromethoxy.

In some embodiments, o=1; p=1; R⁷ is chloro, and R⁶ is trifluoromethoxy.

In some embodiments, at least one R⁷ is C₁-C₆ alkoxy; and at least one R⁶ is halo.

In some embodiments, o=1; p=2; at least one R⁷ is C₁-C₆ alkoxy; and R⁶ is chloro.

In some embodiments, at least one R⁷ is C₃-C₇ cycloalkyl; and at least one R⁶ is C₁-C₆ haloalkyl optionally substituted with hydroxy.

In some embodiments, at least one R⁷ is cyclopropyl; and at least one R⁶ is trifluoromethyl.

In some embodiments, o=2; p=1; R⁷ is cyclopropyl; one R⁶ is trifluoromethyl; and the other R⁶ is fluoro.

In some embodiments, at least one R⁶ is C₁-C₆ alkyl, and at least one R⁷ is C₆-C₁₀ aryl, wherein the C₆-C₁₀ aryl is optionally substituted or optionally fused as described elsewhere herein.

In some embodiments, at least one R⁶ is isopropyl, and at least one R⁷ is C₆-C₁₀ aryl, wherein the C₆-C₁₀ aryl is optionally substituted as described elsewhere herein.

In some embodiments, at least one R⁶ is isopropyl, and at least one R⁷ is dichlorophenyl (e.g., 3,4-dichlorophenyl).

In some embodiments, at least one R⁶ is isopropyl, and at least one R⁷ is dimethylphenyl (e.g., 3,4-dimethylphenyl).

In some embodiments, at least one R⁶ is isopropyl, and at least one R⁷ is naphthyl (e.g., napthyl substituted with one methoxy).

In some embodiments, at least one R⁷ is isopropyl, and at least one R⁶ is C₆-C₁₀ aryl, wherein the C₆-C₁₀ aryl is optionally fused to a five-to-seven-membered carbocyclic ring or heterocyclic ring containing one or two heteroatoms independently selected from oxygen, sulfur and nitrogen (e.g., R⁷ is

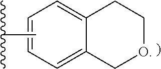

In some embodiments, o=2; p=1; each R⁷ is isopropyl; and R⁶ is C₆-C₁₀ aryl, wherein the C₆-C₁₀ aryl is optionally substituted as described elsewhere herein (e.g., R⁶ is dimethylphenyl; or R⁶ is dichlorophenyl; or R⁶ is naphthyl).

In some embodiments, o=2; p=1, each R⁷ is isopropyl; and R⁶ is C₆-C₁₀ aryl, wherein the C₆-C₁₀ aryl is optionally fused to a five-to-seven-membered carbocyclic ring or heterocyclic ring containing one or two heteroatoms independently selected from oxygen, sulfur and nitrogen (e.g., R⁷ is

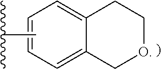

In some embodiments, at least one R⁷ is C₁-C₆ alkyl, and at least one R⁶ is 3- to 7-membered heterocycloalkyl, wherein the 3- to 7-membered heterocycloalkyl is optionally substituted or optionally fused as described elsewhere herein.

In some embodiments, at least one R⁷ is isopropyl, and at least one R⁶ is 3- to 7-membered heterocycloalkyl, wherein the 3- to 7-membered heterocycloalkyl is optionally substituted as described elsewhere herein.

In some embodiments, at least one R⁷ is isopropyl, and at least one R⁶ is tetrahydrofuranyl.

In some embodiments, at least one R⁷ is isopropyl, and at least one R⁶ is

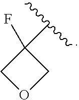 [SMILES: *C1(F)COC1]

In some embodiments, at least one R⁷ is C₁-C₆ alkyl, and at least one R⁶ is 5- to 10-membered heteroaryl, wherein the 5- to 10-membered heteroaryl is optionally substituted or optionally fused as described elsewhere herein.

In some embodiments, at least one R⁷ isopropyl, and at least one R⁶ is 5- to 10-membered heteroaryl, wherein the 5- to 10-membered heteroaryl is optionally substituted or optionally fused as described elsewhere herein.

In some embodiments, at least one R⁷ isopropyl, and at least one R⁶ is pyrazolyl.

In some embodiments, at least one R⁷ is C₁-C₆ alkyl, and at least one R⁶ is C₁-C₆ alkyl substituted with C₃-C₇ cycloalkyl.

In some embodiments, at least one R⁷ is isopropyl, and at least one R⁶ is ethyl substituted with cyclohexyl.

In some embodiments, at least one R⁷ is C₁-C₆ alkyl, and at least one R⁶ is C₁-C₆ alkyl substituted with C₃-C₇ cycloalkyl and oxo.

In some embodiments, at least one R⁷ is isopropyl, and at least one R⁶ is ethyl substituted with cyclohexyl and oxo.

In some embodiments, at least one R⁷ is C₁-C₆ alkyl, and at least one R⁶ is C₁-C₆ alkyl substituted with 4-6 membered heterocycloalkyl (e.g., tetrahydropyranyl).

In some embodiments, at least one R⁷ is C₁-C₆ alkyl, and at least one R⁶ is C₁-C₆ alkyl substituted with C₃-C₇ cycloalkoxy.

In some embodiments, at least one R⁷ is isopropyl, and at least one R⁶ is ethyl substituted with cyclopentoxy.

In some embodiments, at least one R⁷ is C₁-C₆ alkyl, and at least one R⁶ is C₂-C₆ (e.g., C₂) alkynyl substituted with C₃-C₇ cycloalkyl.

In some embodiments, at least one R⁷ is isopropyl, and at least one R⁶ is C₂ alkynyl substituted with cyclohexyl or cyclopentyl.

In some embodiments, at least one R⁷ is C₁-C₆ alkyl, and at least one R⁶ is C₂-C₆ (e.g., C₂) alkynyl substituted with 4-6 membered heterocycloalkyl.

In some embodiments, at least one R⁷ is isopropyl, and at least one R⁶ is C₂ alkynyl substituted with tetrahydropyranyl.

In some embodiments, R⁶ and R⁷ are each attached to a carbon of an aryl ring B.

In some embodiments, R⁶ and R⁷ are each attached to a carbon of a heteroaryl ring B.

In some embodiments, R⁶ is attached to a carbon and R⁷ is attached to a nitrogen of a heteroaryl ring B.

In some embodiments, R⁷ is attached to a carbon and R⁶ is attached to a nitrogen of a heteroaryl ring B.

In some embodiments, one R⁶ and one R⁷ are on adjacent atoms, and taken together with the atoms connecting them, form a C₅ carbocyclic ring optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R⁶ and R⁷ are on adjacent atoms, and taken together with the atoms connecting them, form a C₅ aliphatic carbocyclic ring.

In some embodiments, R⁶ and R⁷ are on adjacent atoms, and taken together with the atoms connecting them, form a C₆ carbocyclic ring optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R⁶ and R⁷ are on adjacent atoms, and taken together with the atoms connecting them, form a C₆ aliphatic carbocyclic ring.

In some embodiments, R⁶ and R⁷ are on adjacent atoms, and taken together with the atoms connecting them, form a C₆ aromatic carbocyclic ring.

In some embodiments, R⁶ and R⁷ are on adjacent atoms, and taken together with the atoms connecting them, form a 5-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R⁶ and R⁷ are on adjacent atoms, and taken together with the atoms connecting them, form a 5-membered aliphatic heterocyclic ring containing 1 or 2 heteroatoms independently selected from O, N, and S.

In some embodiments, R⁶ and R⁷ are on adjacent atoms, and taken together with the atoms connecting them, form a 5-membered heteroaromatic ring containing 1 or 2 heteroatoms independently selected from O, N, and S.

In some embodiments, R⁶ and R⁷ are on adjacent atoms, and taken together with the atoms connecting them, form a 6-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R⁶ and R⁷ are on adjacent atoms, and taken together with the atoms connecting them, form a 6-membered aliphatic heterocyclic ring containing 1 or 2 heteroatoms independently selected from O, N, and S.

In some embodiments, R⁶ and R⁷ are on adjacent atoms, and taken together with the atoms connecting them, form a 6-membered heteroaromatic ring containing 1 or 2 heteroatoms independently selected from O, N, and S.

In some embodiments, one R⁶ and one R⁷ are on adjacent atoms, and taken together with the atoms connecting them, form a C₅-C₈ carbocyclic ring or a 5-to-8-membered heterocyclic ring containing 1 or 2 heteroatoms independently selected from O, N, and S, wherein the ring is fused to the B ring at the ortho- and meta-positions relative to the bond connecting the B ring to the C(R⁴R⁵) group.

In some embodiments, one R⁶ and one R⁷ are on adjacent atoms, and taken together with the atoms connecting them, form a C₅-C₈ carbocyclic ring or a 5-to-8-membered heterocyclic ring containing 1 or 2 heteroatoms independently selected from O, N, and S, wherein the ring is fused to the B ring at the meta- and para-positions relative to the bond connecting the B ring to the C(R⁴R⁵) group.

In some embodiments, o=2; p=2 or 3; and

two pairs, each of one R⁶ and one R⁷, are on adjacent atoms, and each pair of one R⁶ and one R⁷ taken together with the atoms connecting them form a C₅ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them form a C₅ aliphatic carbocyclic ring.

In some embodiments, o=2; p=2 or 3; and

two pairs, each of one R⁶ and one R⁷, are on adjacent atoms, and each pair of one R⁶ and one R⁷ taken together with the atoms connecting them form a C₆ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them form a C₆ aliphatic carbocyclic ring.

In some embodiments, o=2; p=2 or 3; and

two pairs, each of one R⁶ and one R⁷, are on adjacent atoms, and each pair of one R⁶ and one R⁷ taken together with the atoms connecting them form a C₆ aromatic carbocyclic ring.

In some embodiments, o=2; p=2 or 3; and

two pairs, each of one R⁶ and one R⁷, are on adjacent atoms, and each pair of one R⁶ and one R⁷ taken together with the atoms connecting them form a 5-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them form a 5-membered aliphatic heterocyclic ring containing 1 or 2 heteroatoms independently selected from O, N, and S.

In some embodiments, o=2; p=2 or 3; and

two pairs, each of one R⁶ and one R⁷, are on adjacent atoms, and each pair of one R⁶ and one R⁷ taken together with the atoms connecting them form a 5-membered heteroaromatic ring containing 1 or 2 heteroatoms independently selected from O, N, and S.

In some embodiments, o=2; p=2 or 3; and

two pairs, each of one R⁶ and one R⁷, are on adjacent atoms, and each pair of one R⁶ and one R⁷ taken together with the atoms connecting them form a 6-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them form a 6-membered aliphatic heterocyclic ring containing 1 or 2 heteroatoms independently selected from O, N, and S.

In some embodiments, o=2; p=2 or 3; and

two pairs, each of one R⁶ and one R⁷, are on adjacent atoms, and each pair of one R⁶ and one R⁷ taken together with the atoms connecting them form a 6-membered heteroaromatic ring containing 1 or 2 heteroatoms independently selected from O, N, and S.

In some embodiments, o=2; p=2 or 3; and

two pairs, each of one R⁶ and one R⁷, are on adjacent atoms, and each pair of one R⁶ and one R⁷ taken together with the atoms connecting them independently form a C₅-C₈ carbocyclic ring or a 5-to-8-membered heterocyclic ring containing 1 or 2 heteroatoms independently selected from O, N, and S,
wherein one of the two rings is fused to the B ring at the 2- and 3-positions relative to the bond connecting the B ring to the C(R⁴R⁵) group, and the other of the two rings is fused to the B ring at the 5- and 6-positions relative to the bond connecting the B ring to the C(R⁴R⁵) group.

In some embodiments, o=2; p=2; and

two pairs, each of one R⁶ and one R⁷, are on adjacent atoms, and each pair of one R⁶ and one R⁷ taken together with the atoms connecting them form a C₅ aliphatic carbocyclic ring.

In some embodiments, o=2; p=3; and

two pairs, each of one R⁶ and one R⁷, are on adjacent atoms, and each pair of one R⁶ and one R⁷ taken together with the atoms connecting them form a C₅ aliphatic carbocyclic ring; and one R⁷ is halo (e.g., Cl or F).

In some embodiments, o=2; p=3; and

two pairs, each of one R⁶ and one R⁷, are on adjacent atoms, and each pair of one R⁶ and one R⁷ taken together with the atoms connecting them form a C₅ aliphatic carbocyclic ring; and one R⁷ is CN.

In some embodiments, one R⁷ is pyrazolyl and is para to the bond connecting the B ring to the C(R⁴R⁵) group of Formula AA.

In some embodiments, one R⁷ is 3-pyrazolyl and is para to the bond connecting the B ring to the C(R⁴R⁵) group of Formula AA.

In some embodiments, one R⁷ is 4-pyrazolyl and is para to the bond connecting the B ring to the C(R⁴R⁵) group of Formula AA.

In some embodiments, one R⁷ is 5-pyrazolyl and is para to the bond connecting the B ring to the C(R⁴R⁵) group of Formula AA.

In some embodiments, one R⁷ is thiazolyl and is para to the bond connecting the B ring to the C(R⁴R⁵) group of Formula AA.

In some embodiments, one R⁷ is 4-thiazolyl and is para to the bond connecting the B ring to the C(R⁴R⁵) group of Formula AA.

In some embodiments, one R⁷ is 5-thiazolyl and is para to the bond connecting the B ring to the C(R⁴R⁵) group of Formula AA.

In some embodiments, one R⁷ is furyl and is para to the bond connecting the B ring to the C(R⁴R⁵) group of Formula AA.

In some embodiments, one R⁷ is 2-furyl and is para to the bond connecting the B ring to the C(R⁴R⁵) group of Formula AA.

In some embodiments, one R⁷ is thiophenyl and is para to the bond connecting the B ring to the C(R⁴R⁵) group of Formula AA.

In some embodiments, one R⁷ is 2-thiophenyl and is para to the bond connecting the B ring to the C(R⁴R⁵) group of Formula AA.

In some embodiments, one R⁷ is phenyl and is para to the bond connecting the B ring to the C(R⁴R⁵) group of Formula AA.

In some embodiments, one R⁷ is naphthyl (e.g., unsubstituted naphthyl or methoxynaphthyl) and is para to the bond connecting the B ring to the C(R⁴R⁵) group of Formula AA.

In some embodiments, one R⁷ is isochromanyl and is para to the bond connecting the B ring to the C(R⁴R⁵) group of Formula AA.

In some embodiments, one R⁷ is cycloalkenyl (e.g., cyclopentenyl, e.g., 1-cyclopentenyl) and is para to the bond connecting the B ring to the C(R⁴R⁵) group of Formula AA.

In some embodiments, one R⁷ is phenyl optionally substituted with one or more C₁-C₆ alkyl (e.g., methyl or propyl, e.g., 2-propyl) optionally substituted with one or more hydroxyl, NR⁸R⁹ (e.g., dimethylamino), or C₆-C₁₀ aryl (e.g., phenyl, naphthyl, or methylenedioxyphenyl and is para to the bond connecting the B ring to the C(R⁴R⁵) group of Formula AA.

In some embodiments, one R⁷ is phenyl optionally substituted with one or more C₁-C₆ alkoxy (e.g., methoxy) optionally substituted with one or more hydroxyl, NR⁸R⁹ (e.g., dimethylamino), or C₆-C₁₀ aryl (e.g., phenyl, naphthyl, or methylenedioxyphenyl and is para to the bond connecting the B ring to the C(R⁴R⁵) group of Formula AA.

In some embodiments, one R⁷ is phenyl optionally substituted with one or more C₆-C₁₀ aryloxy (e.g., phenoxy) and is para to the bond connecting the B ring to the C(R⁴R⁵) group of Formula AA.

In some embodiments, one R⁷ is phenyl optionally substituted with one or more CN and is para to the bond connecting the B ring to the C(R⁴R⁵) group of Formula AA.

In some embodiments, one R⁷ is phenyl optionally substituted with one or more halo (e.g., F, Cl) and is para to the bond connecting the B ring to the C(R⁴R⁵) group of Formula AA.

In some embodiments, one R⁷ is phenyl optionally substituted with one or more COOC₁-C₆ alkyl (e.g., CO₂t-Bu) and is para to the bond connecting the B ring to the C(R⁴R⁵) group of Formula AA.

In some embodiments, one R⁷ is phenyl optionally substituted with one or more S(O₂)C₁-C₆ alkyl (e.g., S(O₂)methyl) and is para to the bond connecting the B ring to the C(R⁴R⁵) group of Formula AA.

In some embodiments, one R⁷ is phenyl optionally substituted with one or more 3- to 7-membered heterocycloalkyl (e.g., morpholinyl) and is para to the bond connecting the B ring to the C(R⁴R⁵) group of Formula AA.

In some embodiments, one R⁷ is phenyl optionally substituted with one or more CONR⁸R⁹ (e.g., unsubstituted amido) and is para to the bond connecting the B ring to the C(R⁴R⁵) group of Formula AA.

In some embodiments, one R⁷ is phenyl optionally substituted with one or more C₁-C₆ alkyl (e.g., methyl or propyl, e.g., 2-propyl) and with one or more halo (e.g., F, Cl) and is para to the bond connecting the B ring to the C(R⁴R⁵) group of Formula AA.

In some embodiments, one R⁷ is phenyl optionally substituted with one or more C₁-C₆ alkyl (e.g., methyl or propyl, e.g., 2-propyl) optionally substituted with one or more (e.g., one) halo (e.g., fluoro) and is para to the bond connecting the B ring to the C(R⁴R⁵) group of Formula AA.

In some embodiments, R⁶ and R⁷ are each attached to a carbon of an aryl ring B.

In some embodiments, R⁶ and R⁷ are each attached to a carbon of a heteroaryl ring B.

In some embodiments, R⁶ is attached to a carbon and R⁷ is attached to a nitrogen of a heteroaryl ring B.

In some embodiments, R⁷ is attached to a carbon and R⁶ is attached to a nitrogen of a heteroaryl ring B.

In some embodiments, the substituted ring B is

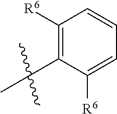

and each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In some embodiments, the substituted ring B is

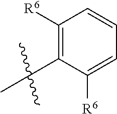

and each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, wherein the C₁-C₆ alkyl, C₁-C₆ haloalkyl, and C₃-C₇ cycloalkyl is optionally substituted with one or more substituents each independently selected from hydroxy, halo, CN, or oxo.

In some embodiments, the substituted ring B is

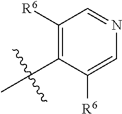

and each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In some embodiments, the substituted ring B is

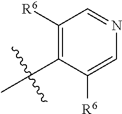

and each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, wherein the C₁-C₆ alkyl, C₁-C₆ haloalkyl, and C₃-C₇ cycloalkyl is optionally substituted with one or more substituents each independently selected from hydroxy, halo, CN, or oxo.

In some embodiments, the substituted ring B is

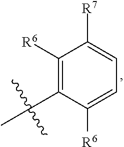

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the substituted ring B is

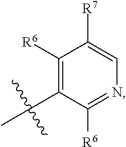

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the substituted ring B is

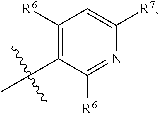

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.

In some embodiments, the substituted ring B is

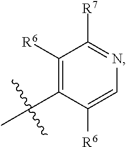

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the substituted ring B is

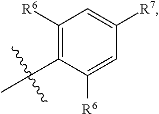

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.

In some embodiments, the substituted ring B is

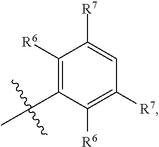

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the substituted ring B is

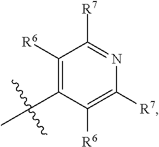

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the substituted ring B is

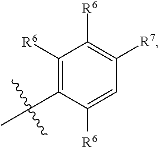

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the substituted ring B is

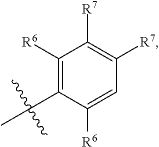

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the substituted ring B is

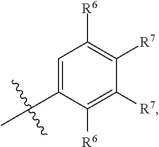

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, the substituted ring B is

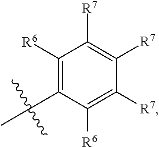

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, the substituted ring B is

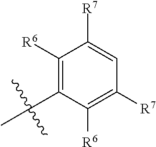

In certain of the foregoing embodiments, each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of these embodiments, each pair of R⁶ and R⁷ on adjacent atoms, taken together with the atoms connecting them, independently forms a C₄-C₇ (e.g., C₄-C₈ (e.g., C₅)) carbocyclic ring.

In certain of the foregoing embodiments, one pair of R⁶ and R⁷ on adjacent atoms, taken together with the atoms connecting them, independently forms a C₅ carbocyclic ring.

In certain of these embodiments, the second pair of R⁶ and R⁷ on adjacent atoms, taken together with the atoms connecting them, independently forms a C₄-C₈ (e.g., C₄ or C₅) carbocyclic ring.

In certain embodiments (when each pair of R⁶ and R⁷ on adjacent atoms, taken together with the atoms connecting them, independently forms a C₄-C₇ carbocyclic ring, each pair of R⁶ and R⁷ on adjacent atoms, taken together with the atoms connecting them, independently forms a C₄ carbocyclic ring.

In some embodiments, wherein the substituted ring B is

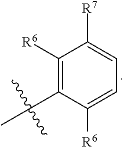

In certain of the foregoing embodiments, each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;

or R⁶ and R⁷ on adjacent atoms, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the substituted ring B is

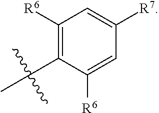

In certain of the foregoing embodiments, each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl, C₆-C₁₀ aryl, and 5- to 10-membered heteroaryl, and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy.

In certain of the foregoing embodiments, one R⁶ is C₁-C₆ alkyl (e.g., isopropyl).

In certain of these embodiments, the other R⁶ is C₁-C₆ alkyl. For example, each R⁶ is isopropyl (i.e., the substituted ring B is

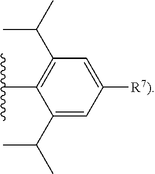

In certain other embodiments, one R⁶ is C₁-C₆ alkyl; and the other R⁶ is C₆-C₁₀ aryl or 5- to 10-membered heteroaryl, each of which is optionally substituted with one or more substituents each independently selected from: hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of these embodiments, one R⁶ is C₁-C₆ alkyl; and the other R⁶ is C₆-C₁₀ aryl or 5- to 10-membered heteroaryl optionally substituted with a substituent selected from halo, CN, C₁-C₆ alkyl, and C₁-C₆ alkoxy. For example, R⁶ is 5-6 (e.g., 6) membered heteroaryl (e.g., pyridinyl (e.g., pyridin-4-yl), pyrimidinyl, pyridazinyl, oxazolyl, or thiazolyl) optionally substituted with a substituent selected from halo, CN, C₁-C₆ alkyl, and C₁-C₆ alkoxy.

As a non-limiting example of the foregoing embodiments, substituted ring B is selected from:

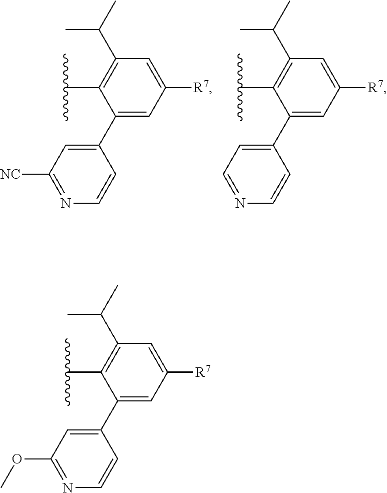

(e.g., R⁷ is halo (e.g., fluoro)).

In some embodiments, B is

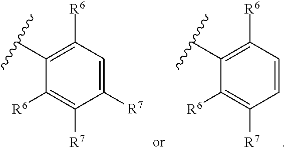

In certain of the foregoing embodiments, each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl, C₆-C₁₀ aryl, and 5- to 10-membered heteroaryl, and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy.

In certain of these embodiments, one R⁶ is C₁-C₆ alkyl; and the other R⁶ is C₆-C₁₀ aryl or 5- to 10-membered heteroaryl, each of which is optionally substituted with one or more substituents each independently selected from: hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of these embodiments, one R⁶ is C₁-C₆ alkyl; and the other R⁶ is C₆-C₁₀ aryl or 5- to 10-membered heteroaryl optionally substituted with a substituent selected from halo, CN, C₁-C₆ alkyl, and C₁-C₆ alkoxy. For example, R⁶ is 5-6 (e.g., 6) membered heteroaryl (e.g., pyridinyl (e.g., pyridin-4-yl), pyrimidinyl, pyridazinyl, oxazolyl, or thiazolyl) optionally substituted with a substituent selected from hydroxyl, halo, CN, C₁-C₆ alkyl, and C₁-C₆ alkoxy.

In certain of the foregoing embodiments, each R⁷ is independently C₁-C₆ alkyl, C₁-C₆ haloalkyl, halo, or cyano,

As a non-limiting example of the foregoing embodiments, substituted ring B is:

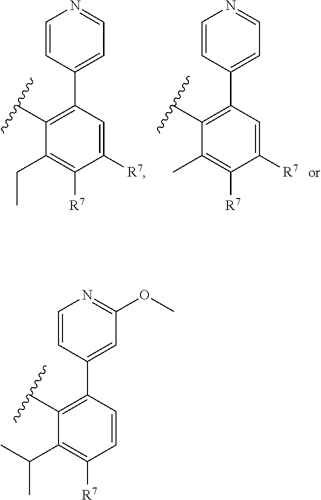

(e.g., each R⁷ is independently C₁-C₆ alkyl, C₁-C₆ haloalkyl, halo, or cyano).

In some embodiments, the substituted ring B is

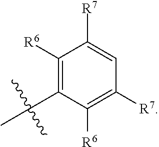

In certain of the foregoing embodiments, each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;

or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of these embodiments, each pair of R⁶ and R⁷ on adjacent atoms, taken together with the atoms connecting them, independently forms a C₄-C₇ (e.g., C₄-C₈ (e.g., C₅)) carbocyclic ring.

In certain embodiments the substituted ring B is

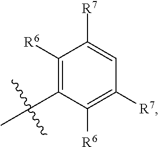

one pair of R⁶ and R⁷ on adjacent atoms, taken together with the atoms connecting them, independently forms a C₅ carbocyclic ring.

In certain of the foregoing embodiments, the second pair of R⁶ and R⁷ on adjacent atoms, taken together with the atoms connecting them, independently forms a C₄-C₅ (e.g., C₄ or C₅) carbocyclic ring.

In certain embodiments the substituted ring B is

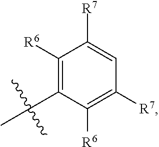

each pair of R⁶ and R⁷ on adjacent atoms, taken together with the atoms connecting them, independently forms a C₄ carbocyclic ring.

In some embodiments, the substituted ring B is

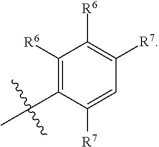

In certain of the foregoing embodiments, each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;

or R⁶ and R⁷ on adjacent atoms, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of the foregoing embodiments, R⁶ and R⁷ on adjacent atoms, taken together with the atoms connecting them, independently form C₄-C₇ (e.g., C₅) carbocyclic ring.

In certain of these embodiments, R⁶ and R⁷ on adjacent atoms, together with the atoms connecting them, independently form a 5-to-7-membered heterocyclic ring containing an O atom.

In certain of the foregoing embodiments, each of the remaining R⁶ and R⁷ is independently selected from C₁-C₆ alkyl (e.g., isopropyl or ethyl (e.g., isopropyl)).

In some embodiments, the substituted ring B is

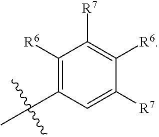

In certain of the foregoing embodiments, each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;

or R⁶ and R⁷ on adjacent atoms, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of these embodiments, each pair of R⁶ and R⁷ on adjacent atoms, taken together with the atoms connecting them, independently forms a C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

As a non-limiting example of the foregoing embodiments, each pair of R⁶ and R⁷ on adjacent atoms, taken together with the atoms connecting them, independently forms a C₄-C₇ (e.g., C₄-C₈ (e.g., C₅)) carbocyclic ring.

In some embodiments, the substituted ring B is

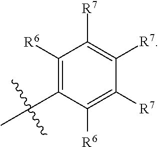

In certain of the foregoing embodiments, each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
   wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;

or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of these embodiments, each pair of R⁶ and R⁷ on adjacent atoms, taken together with the atoms connecting them, independently forms a C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

As a non-limiting example of the foregoing embodiments, each pair of R⁶ and R⁷ on adjacent atoms, taken together with the atoms connecting them, independently forms a C₄-C₇ (e.g., C₄-C₈ (e.g., C₅)) carbocyclic ring.

In certain of the foregoing embodiments, the remaining R⁷ is independently cyano or halo (e.g., halo (e.g., F)).

In some embodiments, the substituted ring B is

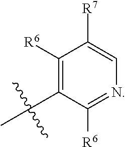

In some embodiments, the substituted ring B is

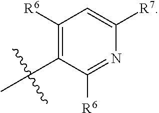

In some embodiments, the substituted ring B is

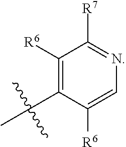

In certain of the foregoing embodiments (when the substituted ring B is

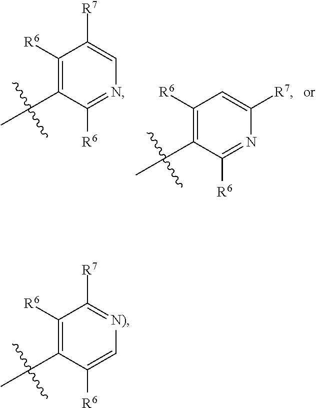

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein the C₆-C₁₀ aryl is optionally substituted with one to two C₁-C₆ alkyl optionally substituted with one to three halo; and wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;

or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain embodiments, each R⁶ and R⁷ is independently C₁-C₆ alkyl, C₁-C₆ haloalkyl, halo, —CN, C₃-C₇ cycloalkyl.

In some embodiments, the substituted ring B is

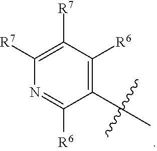

In certain of these embodiments, each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein the C₆-C₁₀ aryl is optionally substituted with one to two C₁-C₆ alkyl optionally substituted with one to three halo; and wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;

or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In In some embodiments, the substituted ring B is selected from:

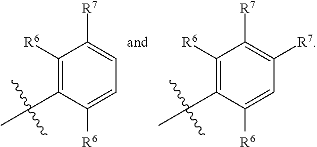

In certain of the foregoing embodiments, each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein each of the C₁-C₆ alkyl, C₁-C₆ haloalkyl, C₃-C₇ cycloalkyl, 4- to 6-membered heterocycloalkyl, C₆-C₁₀ aryl, and 5- to 10-membered heteroaryl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;

or R⁶ and R⁷ on adjacent atoms, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of these embodiments, the R⁶ and R⁷ on adjacent atoms, taken together with the atoms connecting them, independently form C₄-C₇ (e.g., C₄ or C₅) carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. For example, the R⁶ and R⁷ on adjacent atoms, taken together with the atoms connecting them, independently form C₄-C₇ (e.g., C₅) carbocyclic ring. For example, the substituted ring B is

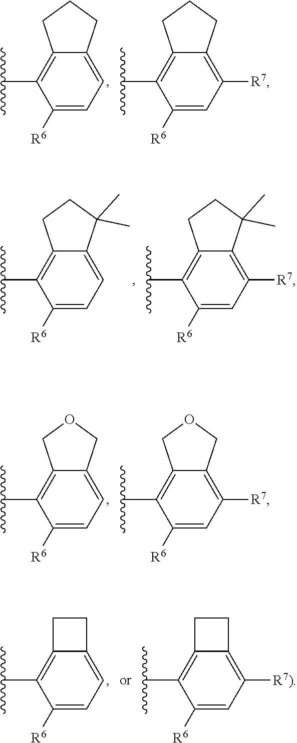

In certain embodiments (when the substituted ring B is selected from:

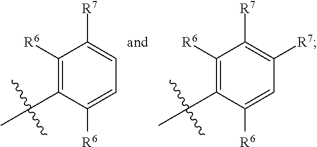

and the R⁶ and R⁷ on adjacent atoms, taken together with the atoms connecting them, independently form C₄-C₇ (e.g., C₄ or C₅) carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹):

the remaining R⁶ is C₆-C₁₀ aryl or 5- to 10-membered heteroaryl, each of which is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of these embodiments, the remaining R⁶ is C₆-C₁₀ aryl or 5- to 10-membered heteroaryl optionally substituted with a substituent selected from halo, CN, C₁-C₆ alkyl, and C₁-C₆ alkoxy. For example, R⁶ is 5-6 membered heteroaryl (e.g., pyridinyl (e.g., pyridin-4-yl), pyrimidinyl, pyridazinyl, oxazolyl, or thiazolyl) optionally substituted with a substituent selected from halo, CN, C₁-C₆ alkyl, and C₁-C₆ alkoxy.

As a non-limiting example of the foregoing embodiments, substituted ring B is selected from:

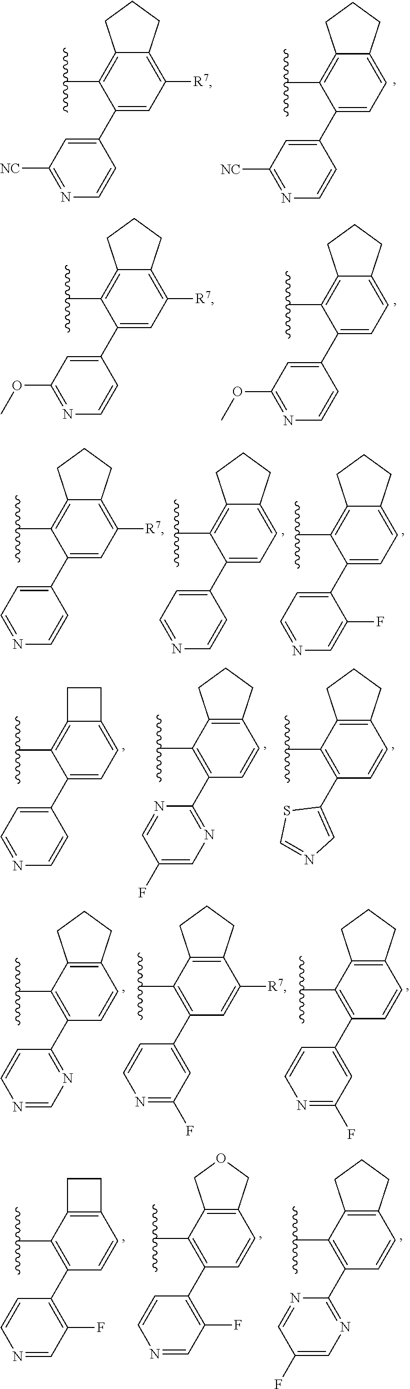

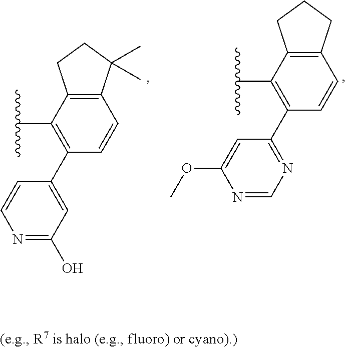 [SMILES: *c1c(-c2ccnc(O)c2)ccc2c1CCC2(C)C ; *c1c(-c2cc(OC)ncn2)ccc2c1CCC2]

In some embodiments, substituted ring B is selected from:

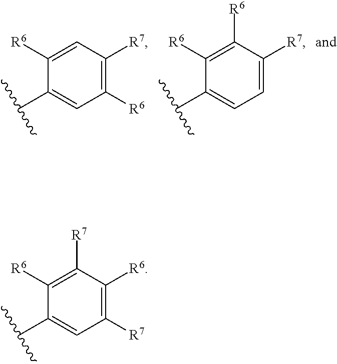

In certain of the foregoing embodiments, each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein each of the C₁-C₆ alkyl, C₁-C₆ haloalkyl, C₃-C₇ cycloalkyl, 4- to 6-membered heterocycloalkyl, C₆-C₁₀ aryl, and 5- to 10-membered heteroaryl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;

or one pair R⁶ and R⁷ on adjacent atoms, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of these embodiments, one pair R⁶ and R⁷ on adjacent atoms, taken together with the atoms connecting them, independently form C₄-C₇ (e.g., C₄ or C₅) carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. For example, the R⁶ and R⁷ on adjacent atoms, taken together with the atoms connecting them, independently form C₄-C₇ (e.g., C₅) carbocyclic ring. For example, the substituted ring B is:

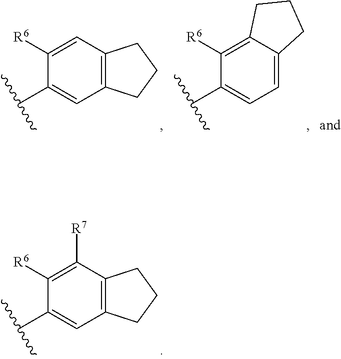

In certain embodiments (when the substituted ring B is selected from:

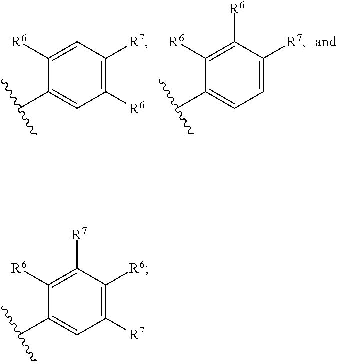

and one pair of R⁶ and R⁷ on adjacent atoms, taken together with the atoms connecting them, independently form C₄-C₇ (e.g., C₄ or C₅) carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹):

the remaining R⁶ is C₆-C₁₀ aryl or 5- to 10-membered heteroaryl, each of which is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of these embodiments, the remaining R⁶ is C₆-C₁₀ aryl or 5- to 10-membered heteroaryl optionally substituted with a substituent selected from halo, CN, C₁-C₆ alkyl, and C₁-C₆ alkoxy. For example, R⁶ is 5-6 membered heteroaryl (e.g., pyridinyl (e.g., pyridin-4-yl), pyrimidinyl, pyridazinyl, oxazolyl, or thiazolyl) optionally substituted with a substituent selected from halo, CN, C₁-C₆ alkyl, and C₁-C₆ alkoxy.

As a non-limiting example of the foregoing embodiments, substituted ring B is selected from:

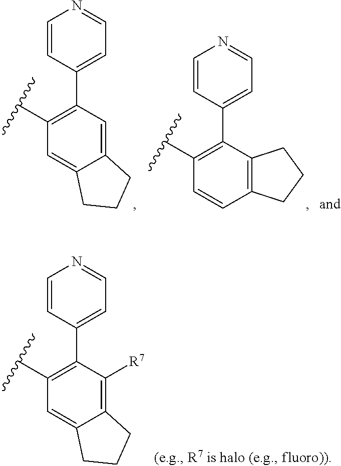

In some embodiments, the substituted ring B is

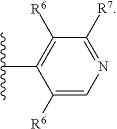

In certain of the foregoing embodiments, each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein each of the C₁-C₆ alkyl, C₁-C₆ haloalkyl, C₃-C₇ cycloalkyl, 4- to 6-membered heterocycloalkyl, C₆-C₁₀ aryl, and 5- to 10-membered heteroaryl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;

or R⁶ and R⁷ on adjacent atoms, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of these embodiments, the R⁶ and R⁷ on adjacent atoms, taken together with the atoms connecting them, independently form C₄-C₇ (e.g., C₄ or C₅) carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. For example, the R⁶ and R⁷ on adjacent atoms, taken together with the atoms connecting them, independently form C₄-C₇ (e.g., C₅) carbocyclic ring. For example, the substituted ring B is

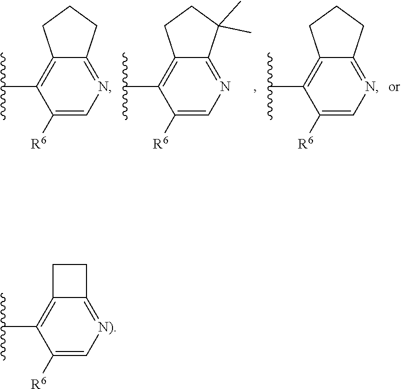

In certain embodiments (when the substituted ring B is selected from:

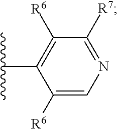

and the R⁶ and R⁷ on adjacent atoms, taken together with the atoms connecting them, independently form C₄-C₇ (e.g., C₄ or C₅) carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹):

the remaining R⁶ is C₆-C₁₀ aryl or 5- to 10-membered heteroaryl, each of which is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of these embodiments, the remaining R⁶ is C₆-C₁₀ aryl or 5- to 10-membered heteroaryl optionally substituted with a substituent selected from halo, CN, C₁-C₆ alkyl, and C₁-C₆ alkoxy. For example, R⁶ is 5-6 membered heteroaryl (e.g., pyridinyl (e.g., pyridin-4-yl), pyrimidinyl, pyridazinyl, oxazolyl, or thiazolyl) optionally substituted with a substituent selected from halo, CN, C₁-C₆ alkyl, and C₁-C₆ alkoxy.

As a non-limiting example of the foregoing embodiments, substituted ring B is selected from:

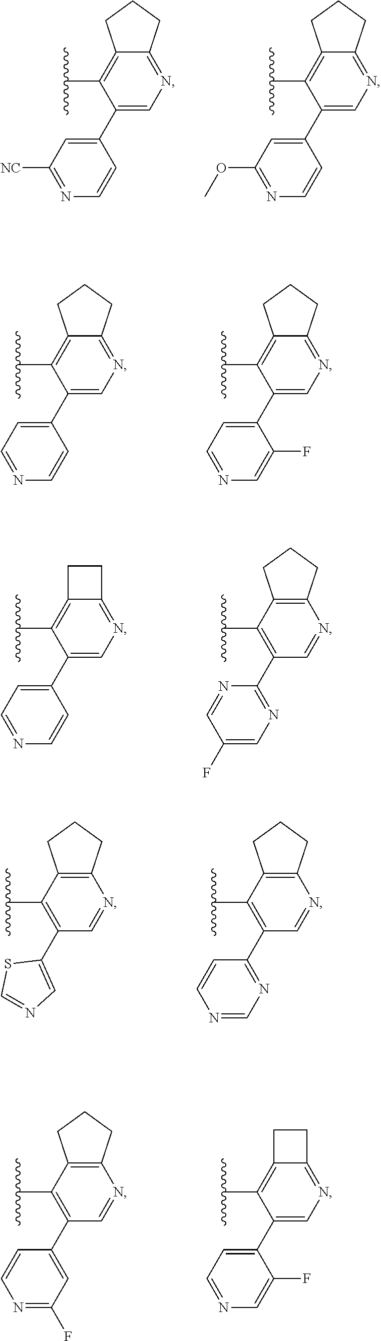

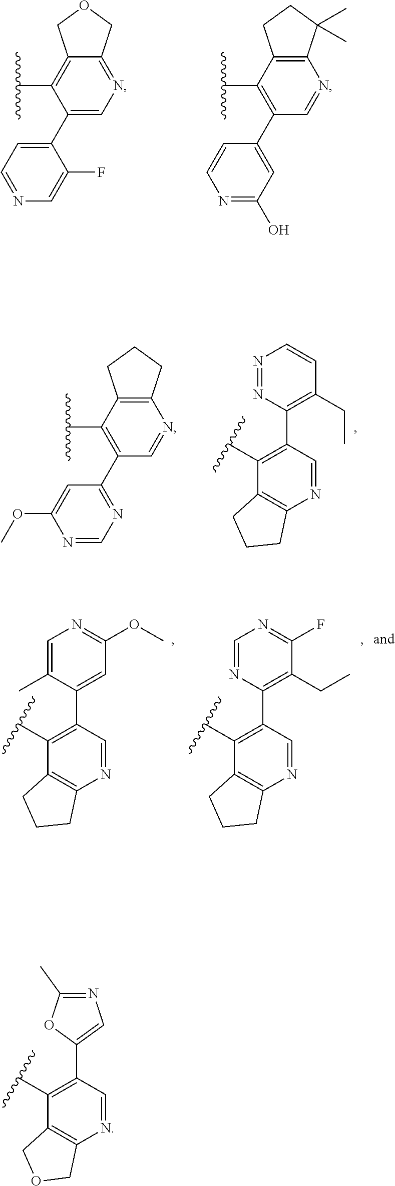

In certain embodiments (when the substituted ring B is

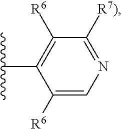

one R⁶ is C₆-C₁₀ aryl or 5- to 10-membered heteroaryl, each of which is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of these embodiments, one R⁶ is C₆-C₁₀ aryl or 5- to 10-membered heteroaryl optionally substituted with a substituent selected from halo, CN, C₁-C₆ alkyl, and C₁-C₆ alkoxy. For example, R⁶ is 5-6 membered heteroaryl (e.g., pyridinyl (e.g., pyridin-4-yl), pyrimidinyl, pyridazinyl, oxazolyl, or thiazolyl) optionally substituted with a substituent selected from halo, CN, C₁-C₆ alkyl, and C₁-C₆ alkoxy.

In certain embodiments (when the substituted ring B is

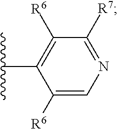

and one R⁶ is C₆-C₁₀ aryl or 5- to 10-membered heteroaryl, each of which is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl):

the remaining R⁶ and R⁷ are independently selected from the group consisting of cyano, halo, C₁-C₆ alkyl, C₁-C₆ haloalkyl, C₁-C₆ alkoxy, and C₃-C₇ cycloalkyl.

As non-limiting examples of the foregoing embodiments, B is:

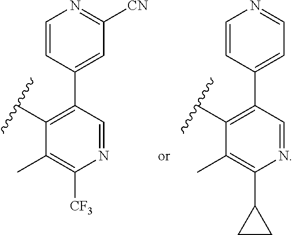 [SMILES: *c1c(-c2ccnc(C#N)c2)cnc(C(F)(F)F)c1C ; *c1c(-c2ccncc2)cnc(C2CC2)c1C]

In some embodiments, the substituted ring B is

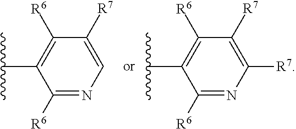

In certain of the foregoing embodiments, each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein each of the C₁-C₆ alkyl, C₁-C₆ haloalkyl, C₃-C₇ cycloalkyl, 4- to 6-membered heterocycloalkyl, C₆-C₁₀ aryl, and 5- to 10-membered heteroaryl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;

or R⁶ and R⁷ on adjacent atoms, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of these embodiments, the R⁶ and R⁷ on adjacent atoms, taken together with the atoms connecting them, independently form C₄-C₇ (e.g., C₄ or C₅) carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. For example, the R⁶ and R⁷ on adjacent atoms, taken together with the atoms connecting them, independently form C₄-C₇ (e.g., C₅) carbocyclic ring. For example, the substituted ring B is

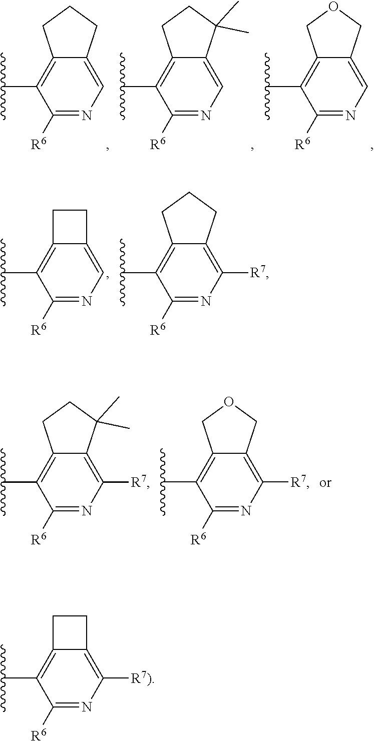

In certain embodiments (when the substituted ring B is

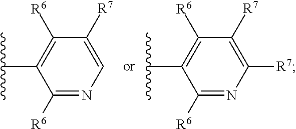

and the R⁶ and R⁷ on adjacent atoms, taken together with the atoms connecting them, independently form C₄-C₇ (e.g., C₄ or C₅) carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹):

the remaining R⁶ is C₆-C₁₀ aryl or 5- to 10-membered heteroaryl, each of which is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of these embodiments, the remaining R⁶ is C₆-C₁₀ aryl or 5- to 10-membered heteroaryl optionally substituted with a substituent selected from halo, CN, C₁-C₆ alkyl, and C₁-C₆ alkoxy. For example, R⁶ is 5-6 membered heteroaryl (e.g., pyridinyl (e.g., pyridin-4-yl), pyrimidinyl, pyridazinyl, oxazolyl, or thiazolyl) optionally substituted with a substituent selected from halo, CN, C₁-C₆ alkyl, and C₁-C₆ alkoxy.

As a non-limiting example of the foregoing embodiments, substituted ring B is selected from:

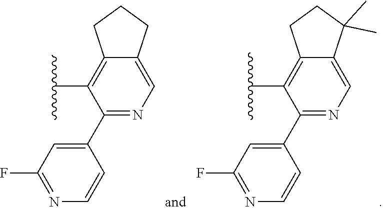 [SMILES: *c1c(-c2ccnc(F)c2)ncc2c1CCC2 ; *c1c(-c2ccnc(F)c2)ncc2c1CCC2(C)C]

In certain embodiments (when the substituted ring B is

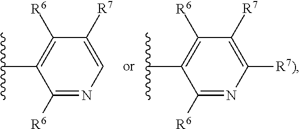

one R⁶ is C₆-C₁₀ aryl or 5- to 10-membered heteroaryl, each of which is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of these embodiments, one R⁶ is C₆-C₁₀ aryl or 5- to 10-membered heteroaryl optionally substituted with a substituent selected from halo, CN, C₁-C₆ alkyl, and C₁-C₆ alkoxy. For example, R⁶ is 5-6 membered heteroaryl (e.g., pyridinyl (e.g., pyridin-4-yl), pyrimidinyl, pyridazinyl, oxazolyl, or thiazolyl) optionally substituted with a substituent selected from halo, CN, C₁-C₆ alkyl, and C₁-C₆ alkoxy.

In certain embodiments (when the substituted ring B is

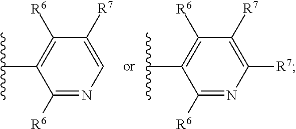

and one R⁶ is C₆-C₁₀ aryl or 5- to 10-membered heteroaryl, each of which is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl):

the remaining R⁶ and each R⁷ are independently selected from the group consisting of cyano, halo, C₁-C₆ alkyl, C₁-C₆ haloalkyl, C₁-C₆ alkoxy, and C₃-C₇ cycloalkyl.

As non-limiting examples of the foregoing embodiments, B is:

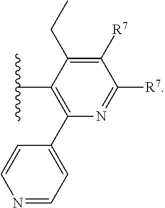

In some embodiments, the substituted ring B is

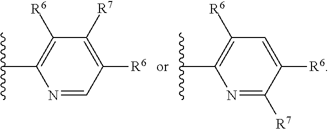

In certain of the foregoing embodiments, each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein each of the C₁-C₆ alkyl, C₁-C₆ haloalkyl, C₃-C₇ cycloalkyl, 4- to 6-membered heterocycloalkyl, C₆-C₁₀ aryl, and 5- to 10-membered heteroaryl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;

or R⁶ and R⁷ on adjacent atoms, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of these embodiments, the R⁶ and R⁷ on adjacent atoms, taken together with the atoms connecting them, independently form C₄-C₇ (e.g., C₄ or C₅) carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. For example, the R⁶ and R⁷ on adjacent atoms, taken together with the atoms connecting them, independently form C₄-C₇ (e.g., C₅) carbocyclic ring. For example, the substituted ring B is

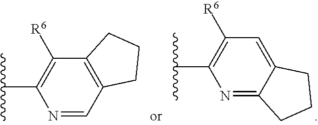

In certain embodiments (when the substituted ring B is

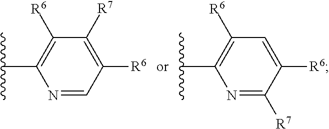

and one pair of R⁶ and R⁷ on adjacent atoms, taken together with the atoms connecting them, independently form C₄-C₇ (e.g., C₄ or C₅) carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹):

the remaining R⁶ is C₆-C₁₀ aryl or 5- to 10-membered heteroaryl, each of which is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of these embodiments, the remaining R⁶ is C₆-C₁₀ aryl or 5- to 10-membered heteroaryl optionally substituted with a substituent selected from halo, CN, C₁-C₆ alkyl, and C₁-C₆ alkoxy. For example, R⁶ is 5-6 membered heteroaryl (e.g., pyridinyl (e.g., pyridin-4-yl), pyrimidinyl, pyridazinyl, oxazolyl, or thiazolyl) optionally substituted with a substituent selected from halo, CN, C₁-C₆ alkyl, and C₁-C₆ alkoxy.

As a non-limiting example of the foregoing embodiments, substituted ring B is selected from:

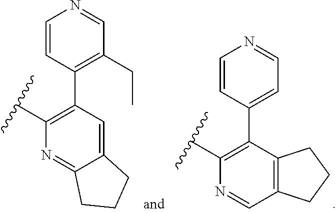 [SMILES: *c1nc2c(cc1-c1ccncc1CC)CCC2 ; *c1ncc2c(c1-c1ccncc1)CCC2]

The Groups R⁴ and R⁵

In some embodiments, each of R⁴ and R⁵ is independently selected from hydrogen and C₁-C₆ alkyl.

In some embodiments, R⁴ is hydrogen.

In some embodiments, R⁵ is hydrogen.

In some embodiments, each of R⁴ and R⁵ is hydrogen.

In some embodiments, R⁴ is C₁-C₆ alkyl.

In some embodiments, R⁵ is C₁-C₆ alkyl.

In some embodiments, each of R⁴ and R⁵ is C₁-C₆ alkyl,

In some embodiments, R⁴ is hydrogen and R⁵ is C₁-C₆ alkyl.

In some embodiments, R⁴ is hydrogen; R⁵ is C₁-C₆ alkyl; and the carbon bonded to R⁴ and R⁵ has (S) stereochemistry.

In some embodiments, R⁴ is hydrogen; and R⁵ is C₁-C₆ alkyl; and the carbon bonded to R⁴ and R⁵ has (R) stereochemistry.

### The Group R³

In some embodiments, R³ is selected from hydrogen, C₁-C₆ alkyl, and

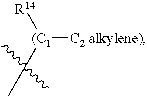

wherein the C₁-C₂ alkylene group is optionally substituted by oxo.

In some embodiments, R³ is hydrogen.

In some embodiments, R³ is cyano.

In some embodiments, R³ is hydroxy.

In some embodiments, R³ is C₁-C₆ alkoxy.

In some embodiments, R³ is C₁-C₆ alkyl.

In some embodiments, R³ is methyl.

In some embodiments, R³ is

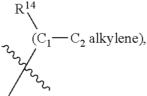

wherein the C₁-C₂ alkylene group is optionally substituted by oxo.

In some embodiments, R³ is —CH₂R¹⁴.

In some embodiments, R³ is —C(O)R¹⁴. In certain of these embodiments, R³ is CHO. In certain embodiments, R³ is C(O)C₁-C₆ alkyl.

In some embodiments, R³ is —CH₂CH₂R¹⁴.

In some embodiments, R³ is —CHR¹⁴CH₃.

In some embodiments, R³ is —CH₂C(O)R¹⁴.

In some embodiments, R³ is —C(O)CH₂R¹⁴.

### The Group R¹⁴

In some embodiments, R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally independently substituted with 1 or 2 R⁶.

In some embodiments, R¹⁴ is hydrogen or C₁-C₆ alkyl.

In some embodiments, R¹⁴ is hydrogen, 5-10-membered monocyclic or bicyclic heteroaryl or C₆-C₁₀ monocyclic or bicyclic aryl, wherein each C₁-C₆ alkyl, aryl or heteroaryl is optionally independently substituted with 1 or 2 R⁶.

In some embodiments, R¹⁴ is hydrogen.

In some embodiments, R¹⁴ is C₁-C₆ alkyl.

In some embodiments, R¹⁴ is methyl.

In some embodiments, R¹⁴ is 5-10-membered monocyclic or bicyclic heteroaryl optionally independently substituted with 1 or 2 R⁶.

In some embodiments, R¹⁴ is C₆-C₁₀ monocyclic or bicyclic aryl optionally independently substituted with 1 or 2 R⁶.

### The Moiety S(═O)(NHR³)═N—

In some embodiments, the sulfur in the moiety S(═O)(NHR³)═N— has (S) stereochemistry.

In some embodiments, the sulfur in the moiety S(═O)(NHR³)═N— has (R) stereochemistry.

### The Group R¹⁰

In some embodiments, R¹⁰ is C₁-C₆ alkyl.

In some embodiments, R¹⁰ is methyl.

In some embodiments, R¹⁰ is ethyl.

The Groups R⁸ and R⁹

In some embodiments, each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to.

In some embodiments, each of R⁸ and R⁹ at each occurrence is hydrogen,

In some embodiments, each R⁸ at each occurrence is hydrogen and each R⁹ at each occurrence is C₁-C₆ alkyl.

In some embodiments, each R⁸ at each occurrence is hydrogen and each R⁹ at each occurrence is methyl.

In some embodiments, each R⁸ at each occurrence is hydrogen and each R⁹ at each occurrence is ethyl.

In some embodiments, each of R⁸ and R⁹ at each occurrence is methyl.

In some embodiments, each of R⁸ and R⁹ at each occurrence is ethyl.

In some embodiments, R⁸ and R⁹ taken together with the nitrogen they are attached to form a 3-membered ring.

In some embodiments, R⁸ and R⁹ taken together with the nitrogen they are attached to form a 4-membered ring.

In some embodiments, R⁸ and R⁹ taken together with the nitrogen they are attached to form a 5-membered ring.

In some embodiments, R⁸ and R⁹ taken together with the nitrogen they are attached to form a 6-membered ring optionally containing one or more oxygen atoms in addition to the nitrogen they are attached to.

In some embodiments, R⁸ and R⁹ taken together with the nitrogen they are attached to form a 6-membered ring optionally containing one or more nitrogen atoms in addition to the nitrogen they are attached to.

In some embodiments, R⁸ and R⁹ taken together with the nitrogen they are attached to form a 7-membered ring.

In some embodiments, one of R⁸ and R⁹ is C(O)R¹³; R¹³ is —(Z¹-Z²)ₐ₁—Z³; and a1 is 0.

In certain of these embodiments, the other one of R⁸ and R⁹ is hydrogen.

As a non-limiting example of the foregoing embodiments, NR⁸R⁹ is selected from the group consisting of: NHCOC₆-C₁₀ aryl, NHCO(5- to 10-membered heteroaryl), NHCO(3- to 7-membered heterocycloalkyl), and NHCOC₂-C₆ alkynyl.

In some embodiments, one of R⁸ and R⁹ is C(O)R¹³; R¹³ is C₁-C₆ alkyl.

In certain embodiments, NR⁸R⁹ is selected from the group consisting of: NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, and NHCOOC₁-C₆ alkyl.

### The Group R³

In some embodiments, R¹³ is C₁-C₆ alkyl.

In some embodiments, R¹³ is methyl.

In some embodiments, R¹³ is ethyl.

In some embodiments, R¹³ is —(Z¹-Z²)ₐ₁—Z³.

In certain of these embodiments, a1 is 0. In certain embodiments, Z³ is C₆-C₁₀ aryl or 5- to 10-membered heteroaryl.

In some embodiments, R¹³ is C₆-C₁₀ aryl.

In some embodiments, R¹³ is phenyl.

In some embodiments, R¹³ is 5- to 10-membered heteroaryl.

The Groups R¹¹ and R¹²

In some embodiments, each of R¹¹ and R¹² at each occurrence is independently selected from hydrogen and C₁-C₆ alkyl.

In some embodiments, each of R¹¹ and R¹² at each occurrence is hydrogen,

In some embodiments, each R¹¹ at each occurrence is hydrogen and each R¹² at each occurrence is C₁-C₆ alkyl.

In some embodiments, each R¹¹ at each occurrence is hydrogen and each R¹² at each occurrence is methyl.

In some embodiments, each R¹¹ at each occurrence is hydrogen and each R¹² at each occurrence is ethyl.

In some embodiments, each of R¹¹ and R¹² at each occurrence is methyl.

In some embodiments, each of R¹¹ and R¹² at each occurrence is ethyl.

### The Group R¹⁵

In some embodiments, R¹⁵ is —(Z⁴-Z⁵)ₐ₂—Z⁶.

In certain embodiments, a2 is 1-5.

In certain embodiments, the Z⁴ group directly attached to R¹ or R² is —O—.

In certain embodiments, each Z⁴ is independently —O— or —NH—, provided that the Z⁴ group directly attached to R¹ or R² is —O—.

In certain embodiments, each Z⁴ is —O—.

In certain embodiments, each Z⁵ is independently C₂-C₆ alkylene optionally substituted with one or more substituents independently selected from oxo, halo, and hydroxyl. In certain these embodiments, each Z⁵ is independently C₂-C₄ (e.g., C₂-C₃ (e.g., C₂ or C₃)) alkylene.

In certain embodiments, Z⁶ is OH.

In certain embodiments, Z⁶ is NHC(O)(C₁-C₆ alkoxy).

In certain embodiments, Z⁶ is C₆-C₁₀ aryl.

In certain embodiments, Z⁶ is C₁-C₆ alkoxy.

In certain embodiments of R¹⁵, a2=1; and Z⁴ is O. In certain of these embodiments, Z⁵ is C₂-C₄ (e.g., C₂-C₃ (e.g., C₂ or C₃)) alkylene. In certain of the foregoing embodiments, Z⁶ is selected from OH, NHC(O)(C1-C6 alkoxy), and C₁-C₆ alkoxy.

As non-limiting examples, R¹⁵ is selected from:

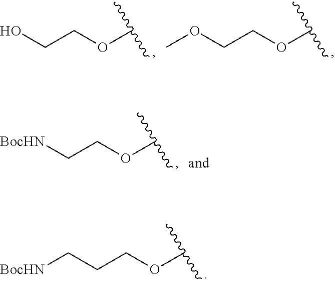

In certain embodiments of R¹⁵, a2=1; and each Z⁴ is O. In certain of these embodiments, Z⁵ is C₂-C₄ (e.g., C₂-C₃ (e.g., C₂ or C₃)) alkylene. In certain of the foregoing embodiments, Z⁶ is selected from OH, NHC(O)(C₁-C₆ alkoxy), and C₁-C₆ alkoxy. In certain other of the foregoing embodiments, Z⁶ is C₆-C₁₀ aryl (e.g., R¹⁵ is

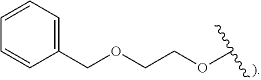 [SMILES: *OCCOCc1ccccc1]

In certain embodiments of R¹⁵, a2≥2 (e.g., a2 is 3 or 4); each Z⁴ is O; and each Z⁵ is ethylene. In certain of these embodiments Z⁶ is OH. In certain other embodiments, Z⁶ is NHC(O)(C₁-C₆ alkoxy) (e.g., Boc). As a non-limiting example, R¹⁵ is:

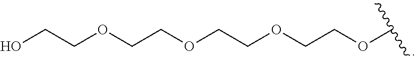 [SMILES: *OCCOCCOCCOCCO]

### Non-Limiting Combinations

In some embodiments of the compound of formula AA,

the substituted ring A is

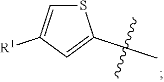

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

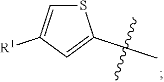

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

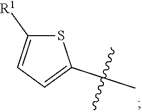

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

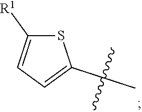

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

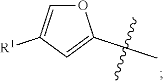

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

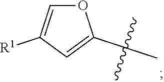

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

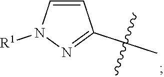

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; and S(O₂)C₁-C₆ alkyl.

In some embodiments of the compound of formula AA,

the substituted ring A is

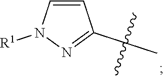

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; phenyl; pyridyl; pyrazolyl; and S(O₂)CH₃.

In some embodiments of the compound of formula AA,

the substituted ring A is

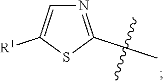

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

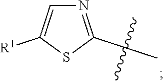

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

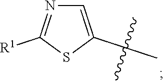

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

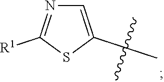

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

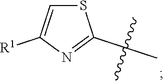

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

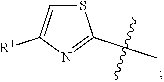

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

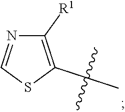

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

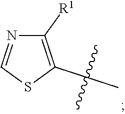

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

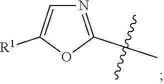

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

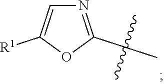

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

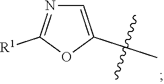

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

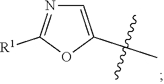

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

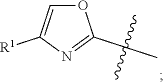

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

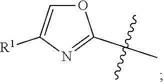

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

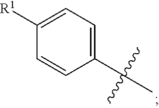

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

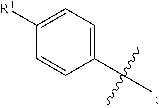

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

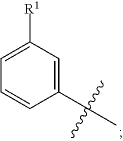

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

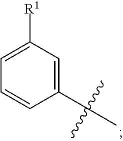

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

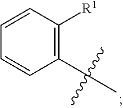

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

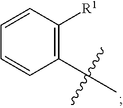

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

### A is

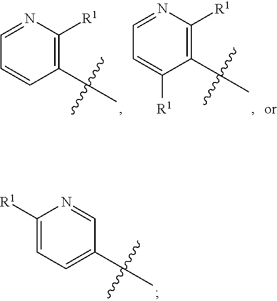

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

### A is

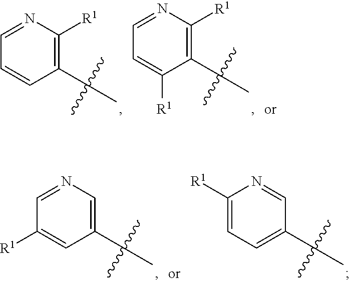

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

### A is

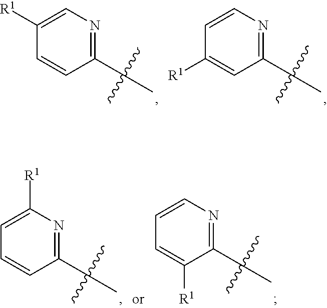

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

### A is

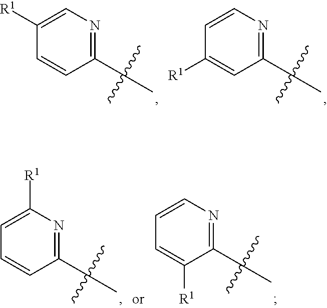

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

### A is

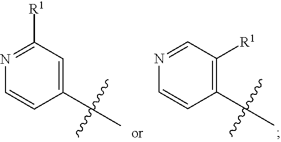

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

### A is

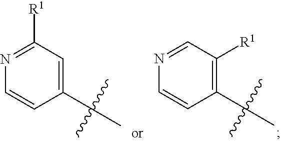

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

### A is

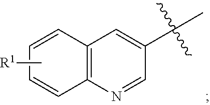

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

### A is

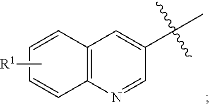

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

### A is

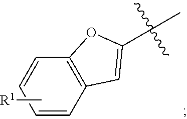

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

### A is

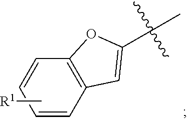

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

### A is

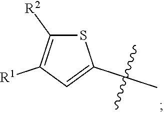

and R¹ and R² are one of the following combinations:
(i) R¹ is C₁-C₆ alkyl optionally substituted with one or more hydroxy, and R² is C₁-C₆ alkyl optionally substituted with one or more hydroxy;
(ii) R¹ is C₁-C₆ alkyl optionally substituted with one or more hydroxy, and R² is C₆-C₁₀ aryl;
(iii) R¹ is C₁-C₆ alkyl optionally substituted with one or more hydroxy, and R² is 5- to 10-membered heteroaryl;
(iv) R¹ is C₁-C₆ alkyl optionally substituted with one or more hydroxy, and R² is SF₅;
(v) R¹ is C₁-C₆ alkyl optionally substituted with one or more hydroxy, and R² is S(O₂)C₁-C₆ alkyl;
(vi) R¹ is C₁-C₆ alkyl optionally substituted with one or more hydroxy, and R² is halo;
(vii) R¹ is C₃-C₇ cycloalkyl optionally substituted with one or more hydroxy, and R² is C₁-C₆ alkyl;
(viii) R¹ is 3- to 7-membered heterocycloalkyl optionally substituted with one or more hydroxy, and R² is C₁-C₆ alkyl;
(ix) R¹ is 3- to 7-membered heterocycloalkyl optionally substituted with one or more hydroxy, and R² is halo;
(x) R¹ is C₁-C₆ alkyl optionally substituted with one or more oxo, and R² is methyl;
(xi) R¹ is C₁-C₆ alkyl optionally substituted with one or more C₁-C₆ alkoxy, and R² is C₁-C₆ alkyl;
(xii) R¹ is C₁-C₆ alkyl optionally substituted with one or more NR⁸R⁹, and R² is C₁-C₆ alkyl;
(xiii) R¹ is C₁-C₆ alkyl optionally substituted with one or more NR⁸R⁹, and R² is halo;
(xiv) R² is C₁-C₆ alkyl optionally substituted with one or more hydroxy, and R¹ is C₆-C₁₀ aryl;
(xv) R² is C₁-C₆ alkyl optionally substituted with one or more hydroxy, and R¹ is 5- to 10-membered heteroaryl;
(xvi) R² is C₁-C₆ alkyl optionally substituted with one or more hydroxy, and R¹ is SF₅.
(xvii) R² is C₁-C₆ alkyl optionally substituted with one or more hydroxy, and R¹ is S(O₂)C₁-C₆ alkyl;
(xviii) R² is C₁-C₆ alkyl optionally substituted with one or more hydroxy, and R¹ is halo;
(xix) R² is C₃-C₇ cycloalkyl optionally substituted with one or more hydroxy, and R¹ is C₁-C₆ alkyl;
(xx) R² is 3- to 7-membered heterocycloalkyl optionally substituted with one or more hydroxy, and R¹ is C₁-C₆ alkyl;
(xxi) R² is 3- to 7-membered heterocycloalkyl optionally substituted with one or more hydroxy, and R¹ is halo;
(xxii) R² is C₁-C₆ alkyl optionally substituted with one or more oxo, and R¹ is methyl;
(xxiii) R² is C₁-C₆ alkyl optionally substituted with one or more C₁-C₆ alkoxy, and R¹ is C₁-C₆ alkyl;
(xxiv) R² is C₁-C₆ alkyl optionally substituted with one or more NR⁸R⁹, and R¹ is C₁-C₆ alkyl;
or
(xxv) R² is C₁-C₆ alkyl optionally substituted with one or more NR⁸R⁹, and R¹ is halo.

In some embodiments of the compound of formula AA,

the substituted ring A is

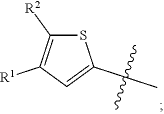

and R¹ and R² are one of the following combinations:
(i) R¹ is 1-hydroxy-2-methylpropan-2-yl, and R² is methyl;
(ii) R¹ is 2-hydroxy-2-propyl and R² is methyl;
(iii) R¹ is 2-hydroxy-2-propyl and R² is isopropyl;
(iv) R¹ is 2-hydroxy-2-propyl and R² is 2-hydroxy-2-propyl;
(v) R¹ is 2-hydroxy-2-propyl and R² is 1-hydroxyethyl;
(vi) R¹ is hydroxymethyl and R² is methyl;
(vii) R¹ is 1-hydroxyethyl and R² is methyl;
(viii) R¹ is 2-hydroxyethyl and R² is methyl;
(ix) R¹ is 1-hydroxy-2-propyl and R² is methyl;
(x) R¹ is 2-hydroxy-2-propyl and R² is phenyl;
(xi) R¹ is 2-hydroxy-2-propyl and R² is pyridyl;
(xii) R¹ is 2-hydroxy-2-propyl and R² is pyrazolyl;
(xiii) R¹ is 2-hydroxy-2-propyl, and R² is S(O₂)CH₃;
(xiv) R¹ is 2-hydroxy-2-propyl and R² is chloro;
(xv) R¹ is 2-hydroxy-2-propyl and R² is fluoro;
(xvi) R¹ is 1-hydroxy-1-cyclopropyl, and R² is methyl;
(xvii) R¹ is 1-hydroxy-1-cyclobutyl, and R² is methyl;
(xviii) R¹ is 1-hydroxy-1-cyclopentyl, and R² is methyl;
(xix) R¹ is 1-hydroxy-1-cyclohexyl, and R² is methyl;
(xx) R¹ is morpholinyl, and R² is methyl;
(xxi) R¹ is 1,3-dioxolan-2-yl, and R² is methyl;
(xxii) R¹ is 1,3-dioxolan-2-yl, and R² is fluoro;
(xxiii) R¹ is 1,3-dioxolan-2-yl, and R² is chloro;
(xxiv) R¹ is COCH₃, and R² is methyl;
(xxv) R¹ is 2-methoxy-2-propyl, and R² is methyl;
(xxvi) R¹ is (dimethylamino)methyl, and R² is methyl;
(xxvii) R² is 1-hydroxy-2-methylpropan-2-yl, and R¹ is methyl;
(xxviii) R² is 2-hydroxy-2-propyl and R¹ is methyl;
(xxix) R² is 2-hydroxy-2-propyl and R¹ is isopropyl;
(xxx) R² is 2-hydroxy-2-propyl and R¹ is 1-hydroxyethyl;
(xxxi) R² is hydroxymethyl and R¹ is methyl;
(xxxii) R² is 1-hydroxyethyl and R¹ is methyl;
(xxxiii) R² is 2-hydroxyethyl and R¹ is methyl;
(xxxiv) R² is 1-hydroxy-2-propyl and R¹ is methyl;
(xxxv) R² is 2-hydroxy-2-propyl and R¹ is phenyl;
(xxxvi) R² is C₁-C₆ alkyl optionally substituted with one or more hydroxy, and R¹ is 5- to 10-membered heteroaryl;
(xxxvii) R² is 2-hydroxy-2-propyl and R¹ is pyridyl;
(xxxviii) R² is 2-hydroxy-2-propyl and R¹ is pyrazolyl;
(xxxix) R² is C₁-C₆ alkyl optionally substituted with one or more hydroxy, and R¹ is S(O₂)CH₃;
(xl) R² is 2-hydroxy-2-propyl and R¹ is chloro;
(xli) R² is 2-hydroxy-2-propyl and R¹ is fluoro;
(xlii) R² is C₃-C₇ cycloalkyl optionally substituted with one or more hydroxy, and R¹ is C₁-C₆ alkyl;
(xliii) R² is 1-hydroxy-1-cyclopropyl, and R¹ is methyl;
(xliv) R² is 1-hydroxy-1-cyclobutyl, and R¹ is methyl;
(xlv) R² is 1-hydroxy-1-cyclopentyl, and R¹ is methyl;
(xlvi) R² is 1-hydroxy-1-cyclohexyl, and R¹ is methyl;
(xlvii) R² is morpholinyl, and R¹ is methyl;
(xlviii) R² is 1,3-dioxolan-2-yl, and R¹ is methyl;
(xlix) R² is 1,3-dioxolan-2-yl, and R¹ is fluoro;
(l) R² is 1,3-dioxolan-2-yl, and R¹ is chloro;
(li) R² is C₁-C₆ alkyl optionally substituted with one or more oxo, and R¹ is methyl;
(lii) R² is COCH₃, and R¹ is methyl;
(liii) R² is 2-methoxy-2-propyl, and R¹ is methyl;
or
(liv) R² is (dimethylamino)methyl, and R¹ is methyl.

In some embodiments, of the compound of formula AA,

the substituted ring A is

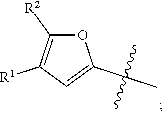

and R¹ and R² are one of the following combinations:
(i) R¹ is C₁-C₆ alkyl optionally substituted with one or more hydroxy, and R² is C₁-C₆ alkyl optionally substituted with one or more hydroxy;
(ii) R¹ is C₁-C₆ alkyl optionally substituted with one or more hydroxy, and R² is C₆-C₁₀ aryl;
(iii) R¹ is C₁-C₆ alkyl optionally substituted with one or more hydroxy, and R² is 5- to 10-membered heteroaryl;
(iv) R¹ is C₁-C₆ alkyl optionally substituted with one or more hydroxy, and R² is SF₅;
(v) R¹ is C₁-C₆ alkyl optionally substituted with one or more hydroxy, and R² is S(O₂)C₁-C₆ alkyl;
(vi) R¹ is C₁-C₆ alkyl optionally substituted with one or more hydroxy, and R² is halo;
(vii) R¹ is C₃-C₇ cycloalkyl optionally substituted with one or more hydroxy, and R² is C₁-C₆ alkyl;
(viii) R¹ is 3- to 7-membered heterocycloalkyl optionally substituted with one or more hydroxy, and R² is C₁-C₆ alkyl;
(ix) R¹ is 3- to 7-membered heterocycloalkyl optionally substituted with one or more hydroxy, and R² is halo;
(x) R¹ is C₁-C₆ alkyl optionally substituted with one or more oxo, and R² is methyl;
(xi) R¹ is C₁-C₆ alkyl optionally substituted with one or more C₁-C₆ alkoxy, and R² is C₁-C₆ alkyl;
(xii) R¹ is C₁-C₆ alkyl optionally substituted with one or more NR⁸R⁹, and R² is C₁-C₆ alkyl;
(xiii) R¹ is C₁-C₆ alkyl optionally substituted with one or more NR⁸R⁹, and R² is halo;
(xiv) R² is C₁-C₆ alkyl optionally substituted with one or more hydroxy, and R¹ is C₆-C₁₀ aryl;
(xv) R² is C₁-C₆ alkyl optionally substituted with one or more hydroxy, and R¹ is 5- to 10-membered heteroaryl;
(xvi) R² is C₁-C₆ alkyl optionally substituted with one or more hydroxy, and R¹ is SF₅.
(xvii) R² is C₁-C₆ alkyl optionally substituted with one or more hydroxy, and R¹ is S(O₂)C₁-C₆ alkyl;
(xviii) R² is C₁-C₆ alkyl optionally substituted with one or more hydroxy, and R¹ is halo;
(xix) R² is C₃-C₇ cycloalkyl optionally substituted with one or more hydroxy, and R¹ is C₁-C₆ alkyl;
(xx) R² is 3- to 7-membered heterocycloalkyl optionally substituted with one or more hydroxy, and R¹ is C₁-C₆ alkyl;
(xxi) R² is 3- to 7-membered heterocycloalkyl optionally substituted with one or more hydroxy, and R¹ is halo;
(xxii) R² is C₁-C₆ alkyl optionally substituted with one or more oxo, and R¹ is methyl;
(xxiii) R² is C₁-C₆ alkyl optionally substituted with one or more C₁-C₆ alkoxy, and R¹ is C₁-C₆ alkyl;
(xxiv) R² is C₁-C₆ alkyl optionally substituted with one or more NR⁸R⁹, and R¹ is C₁-C₆ alkyl;
or
(xxv) R² is C₁-C₆ alkyl optionally substituted with one or more NR⁸R⁹, and R¹ is halo.

In some embodiments, of the compound of formula AA,

the substituted ring A is

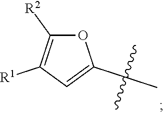

and R¹ and R² are one of the following combinations:
(i) R¹ is 1-hydroxy-2-methylpropan-2-yl, and R² is methyl;
(ii) R¹ is 2-hydroxy-2-propyl and R² is methyl;
(iii) R¹ is 2-hydroxy-2-propyl and R² is isopropyl;
(iv) R¹ is 2-hydroxy-2-propyl and R² is 2-hydroxy-2-propyl;
(v) R¹ is 2-hydroxy-2-propyl and R² is 1-hydroxyethyl;
(vi) R¹ is hydroxymethyl and R² is methyl;
(vii) R¹ is 1-hydroxyethyl and R² is methyl;
(viii) R¹ is 2-hydroxyethyl and R² is methyl;
(ix) R¹ is 1-hydroxy-2-propyl and R² is methyl;
(x) R¹ is 2-hydroxy-2-propyl and R² is phenyl;
(xi) R¹ is 2-hydroxy-2-propyl and R² is pyridyl;
(xii) R¹ is 2-hydroxy-2-propyl and R² is pyrazolyl;
(xiii) R¹ is 2-hydroxy-2-propyl, and R² is S(O₂)CH₃;
(xiv) R¹ is 2-hydroxy-2-propyl and R² is chloro;
(xv) R¹ is 2-hydroxy-2-propyl and R² is fluoro;
(xvi) R¹ is 1-hydroxy-1-cyclopropyl, and R² is methyl;
(xvii) R¹ is 1-hydroxy-1-cyclobutyl, and R² is methyl;
(xviii) R¹ is 1-hydroxy-1-cyclopentyl, and R² is methyl;
(xix) R¹ is 1-hydroxy-1-cyclohexyl, and R² is methyl;
(xx) R¹ is morpholinyl, and R² is methyl;
(xxi) R¹ is 1,3-dioxolan-2-yl, and R² is methyl;
(xxii) R⁸ is 1,3-dioxolan-2-yl, and R² is fluoro;
(xxiii) R¹ is 1,3-dioxolan-2-yl, and R² is chloro;
(xxiv) R¹ is COCH₃, and R² is methyl;
(xxv) R¹ is 2-methoxy-2-propyl, and R² is methyl;
(xxvi) R¹ is (dimethylamino)methyl, and R² is methyl;
(xxvii) R² is 1-hydroxy-2-methylpropan-2-yl, and R¹ is methyl;
(xxviii) R² is 2-hydroxy-2-propyl and R¹ is methyl;
(xxix) R² is 2-hydroxy-2-propyl and R¹ is isopropyl;
(xxx) R² is 2-hydroxy-2-propyl and R¹ is 1-hydroxyethyl;
(xxxi) R² is hydroxymethyl and R¹ is methyl;
(xxxii) R² is 1-hydroxyethyl and R¹ is methyl;
(xxxiii) R² is 2-hydroxyethyl and R¹ is methyl;
(xxxiv) R² is 1-hydroxy-2-propyl and R¹ is methyl;
(xxxv) R² is 2-hydroxy-2-propyl and R¹ is phenyl;
(xxxvi) R² is C₁-C₆ alkyl optionally substituted with one or more hydroxy, and R¹ is 5- to 10-membered heteroaryl;
(xxxvii) R² is 2-hydroxy-2-propyl and R¹ is pyridyl;
(xxxviii) R² is 2-hydroxy-2-propyl and R¹ is pyrazolyl;
(xxxix) R² is C₁-C₆ alkyl optionally substituted with one or more hydroxy, and R¹ is S(O₂)CH₃;
(xl) R² is 2-hydroxy-2-propyl and R¹ is chloro;
(xli) R² is 2-hydroxy-2-propyl and R¹ is fluoro;
(xlii) R² is C₃-C₇ cycloalkyl optionally substituted with one or more hydroxy, and R¹ is C₁-C₆ alkyl;
(xliii) R² is 1-hydroxy-1-cyclopropyl, and R¹ is methyl;
(xliv) R² is 1-hydroxy-1-cyclobutyl, and R¹ is methyl;
(xlv) R² is 1-hydroxy-1-cyclopentyl, and R¹ is methyl;
(xlvi) R² is 1-hydroxy-1-cyclohexyl, and R¹ is methyl;
(xlvii) R² is morpholinyl, and R¹ is methyl;
(xlviii) R² is 1,3-dioxolan-2-yl, and R¹ is methyl;
(xlix) R² is 1,3-dioxolan-2-yl, and R¹ is fluoro;
(l) R² is 1,3-dioxolan-2-yl, and R¹ is chloro;
(li) R² is C₁-C₆ alkyl optionally substituted with one or more oxo, and R¹ is methyl;
(lii) R² is COCH₃, and R¹ is methyl;
(liii) R² is 2-methoxy-2-propyl, and R¹ is methyl;
or
(liv) R² is (dimethylamino)methyl, and R¹ is methyl.

In some embodiments, of the compound of formula AA,

the substituted ring A is

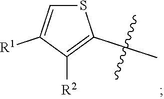

and R¹ and R² are one of the following combinations:
(i) R¹ is C₁-C₆ alkyl optionally substituted with one or more hydroxy, and R² is C₁-C₆ alkyl optionally substituted with one or more hydroxy;
(ii) R¹ is C₁-C₆ alkyl optionally substituted with one or more hydroxy, and R² is C₆-C₁₀ aryl;
(iii) R¹ is C₁-C₆ alkyl optionally substituted with one or more hydroxy, and R² is 5- to 10-membered heteroaryl;
(iv) R¹ is C₁-C₆ alkyl optionally substituted with one or more hydroxy, and R² is SF₅;
(v) R¹ is C₁-C₆ alkyl optionally substituted with one or more hydroxy, and R² is S(O₂)C₁-C₆ alkyl;
(vi) R¹ is C₁-C₆ alkyl optionally substituted with one or more hydroxy, and R² is halo;
(vii) R¹ is C₃-C₇ cycloalkyl optionally substituted with one or more hydroxy, and R² is C₁-C₆ alkyl;
(viii) R¹ is 3- to 7-membered heterocycloalkyl optionally substituted with one or more hydroxy, and R² is C₁-C₆ alkyl;
(ix) R¹ is 3- to 7-membered heterocycloalkyl optionally substituted with one or more hydroxy, and R² is halo;
(x) R¹ is C₁-C₆ alkyl optionally substituted with one or more oxo, and R² is methyl;
(xi) R¹ is C₁-C₆ alkyl optionally substituted with one or more C₁-C₆ alkoxy, and R² is C₁-C₆ alkyl;
(xii) R¹ is C₁-C₆ alkyl optionally substituted with one or more NR⁸R⁹, and R² is C₁-C₆ alkyl;
(xiii) R¹ is C₁-C₆ alkyl optionally substituted with one or more NR⁸R⁹, and R² is halo; (xiv) R² is C₁-C₆ alkyl optionally substituted with one or more hydroxy, and R¹ is C₆-C₁₀ aryl;
(xv) R² is C₁-C₆ alkyl optionally substituted with one or more hydroxy, and R¹ is 5- to 10-membered heteroaryl;
(xvi) R² is C₁-C₆ alkyl optionally substituted with one or more hydroxy, and R¹ is SF₅.
(xvii) R² is C₁-C₆ alkyl optionally substituted with one or more hydroxy, and R¹ is S(O₂)C₁-C₆ alkyl;
(xviii) R² is C₁-C₆ alkyl optionally substituted with one or more hydroxy, and R¹ is halo;
(xix) R² is C₃-C₇ cycloalkyl optionally substituted with one or more hydroxy, and R¹ is C₁-C₆ alkyl;
(xx) R² is 3- to 7-membered heterocycloalkyl optionally substituted with one or more hydroxy, and R¹ is C₁-C₆ alkyl;
(xxi) R² is 3- to 7-membered heterocycloalkyl optionally substituted with one or more hydroxy, and R¹ is halo;
(xxii) R² is C₁-C₆ alkyl optionally substituted with one or more oxo, and R¹ is methyl;
(xxiii) R² is C₁-C₆ alkyl optionally substituted with one or more C₁-C₆ alkoxy, and R¹ is C₁-C₆ alkyl;
(xxiv) R² is C₁-C₆ alkyl optionally substituted with one or more NR⁸R⁹, and R¹ is C₁-C₆ alkyl;
or
(xxv) R² is C₁-C₆ alkyl optionally substituted with one or more NR⁸R⁹, and R¹ is halo.

In some embodiments, of the compound of formula AA,

the substituted ring A is

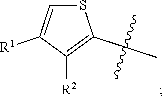

and R¹ and R² are one of the following combinations:
(i) R¹ is 1-hydroxy-2-methylpropan-2-yl, and R² is methyl;
(ii) R¹ is 2-hydroxy-2-propyl and R² is methyl;
(iii) R¹ is 2-hydroxy-2-propyl and R² is isopropyl;
(iv) R¹ is 2-hydroxy-2-propyl and R² is 2-hydroxy-2-propyl;
(v) R¹ is 2-hydroxy-2-propyl and R² is 1-hydroxyethyl;
(vi) R¹ is hydroxymethyl and R² is methyl;
(vii) R¹ is 1-hydroxyethyl and R² is methyl;
(viii) R¹ is 2-hydroxyethyl and R² is methyl;
(ix) R¹ is 1-hydroxy-2-propyl and R² is methyl;
(x) R¹ is 2-hydroxy-2-propyl and R² is phenyl;
(xi) R¹ is 2-hydroxy-2-propyl and R² is pyridyl;
(xii) R¹ is 2-hydroxy-2-propyl and R² is pyrazolyl;
(xiii) R¹ is 2-hydroxy-2-propyl, and R² is S(O₂)CH₃;
(xiv) R¹ is 2-hydroxy-2-propyl and R² is chloro;
(xv) R¹ is 2-hydroxy-2-propyl and R² is fluoro;
(xvi) R¹ is 1-hydroxy-1-cyclopropyl, and R² is methyl;
(xvii) R¹ is 1-hydroxy-1-cyclobutyl, and R² is methyl;
(xviii) R¹ is 1-hydroxy-1-cyclopentyl, and R² is methyl;
(xix) R¹ is 1-hydroxy-1-cyclohexyl, and R² is methyl;
(xx) R¹ is morpholinyl, and R² is methyl;
(xxi) R¹ is 1,3-dioxolan-2-yl, and R² is methyl;
(xxii) R¹ is 1,3-dioxolan-2-yl, and R² is fluoro;
(xxiii) R¹ is 1,3-dioxolan-2-yl, and R² is chloro;
(xxiv) R¹ is COCH₃, and R² is methyl;
(xxv) R¹ is 2-methoxy-2-propyl, and R² is methyl;
(xxvi) R¹ is (dimethylamino)methyl, and R² is methyl;
(xxvii) R² is 1-hydroxy-2-methylpropan-2-yl, and R¹ is methyl;
(xxviii) R² is 2-hydroxy-2-propyl and R¹ is methyl;
(xxix) R² is 2-hydroxy-2-propyl and R¹ is isopropyl;
(xxx) R² is 2-hydroxy-2-propyl and R¹ is 1-hydroxyethyl;
(xxxi) R² is hydroxymethyl and R¹ is methyl;
(xxxii) R² is 1-hydroxyethyl and R¹ is methyl;
(xxxiii) R² is 2-hydroxyethyl and R¹ is methyl;
(xxxiv) R² is 1-hydroxy-2-propyl and R¹ is methyl;
(xxxv) R² is 2-hydroxy-2-propyl and R¹ is phenyl;
(xxxvi) R² is C₁-C₆ alkyl optionally substituted with one or more hydroxy, and R¹ is 5- to 10-membered heteroaryl;
(xxxvii) R² is 2-hydroxy-2-propyl and R¹ is pyridyl;
(xxxviii) R² is 2-hydroxy-2-propyl and R¹ is pyrazolyl;
(xxxix) R² is C₁-C₆ alkyl optionally substituted with one or more hydroxy, and R¹ is S(O₂)CH₃;
(xl) R² is 2-hydroxy-2-propyl and R¹ is chloro;
(xli) R² is 2-hydroxy-2-propyl and R¹ is fluoro;
(xlii) R² is C₃-C₇ cycloalkyl optionally substituted with one or more hydroxy, and R¹ is C₁-C₆ alkyl;
(xliii) R² is 1-hydroxy-1-cyclopropyl, and R¹ is methyl;
(xliv) R² is 1-hydroxy-1-cyclobutyl, and R¹ is methyl;
(xlv) R² is 1-hydroxy-1-cyclopentyl, and R¹ is methyl;
(xlvi) R² is 1-hydroxy-1-cyclohexyl, and R¹ is methyl;
(xlvii) R² is morpholinyl, and R¹ is methyl;
(xlviii) R² is 1,3-dioxolan-2-yl, and R¹ is methyl;
(xlix) R² is 1,3-dioxolan-2-yl, and R¹ is fluoro;
(l) R² is 1,3-dioxolan-2-yl, and R¹ is chloro;
(li) R² is C₁-C₆ alkyl optionally substituted with one or more oxo, and R¹ is methyl;
(lii) R² is COCH₃, and R¹ is methyl;
(liii) R² is 2-methoxy-2-propyl, and R¹ is methyl;
or
(liv) R² is (dimethylamino)methyl, and R¹ is methyl.

In some embodiments, of the compound of formula AA,

the substituted ring A is

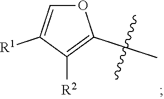

and R¹ and R² are one of the following combinations:
(i) R¹ is C₁-C₆ alkyl optionally substituted with one or more hydroxy, and R² is C₁-C₆ alkyl optionally substituted with one or more hydroxy;
(ii) R¹ is C₁-C₆ alkyl optionally substituted with one or more hydroxy, and R² is C₆-C₁₀ aryl;
(iii) R¹ is C₁-C₆ alkyl optionally substituted with one or more hydroxy, and R² is 5- to 10-membered heteroaryl;
(iv) R¹ is C₁-C₆ alkyl optionally substituted with one or more hydroxy, and R² is SF₅;
(v) R¹ is C₁-C₆ alkyl optionally substituted with one or more hydroxy, and R² is S(O₂)C₁-C₆ alkyl;
(vi) R¹ is C₁-C₆ alkyl optionally substituted with one or more hydroxy, and R² is halo;
(vii) R¹ is C₃-C₇ cycloalkyl optionally substituted with one or more hydroxy, and R² is C₁-C₆ alkyl;
(viii) R¹ is 3- to 7-membered heterocycloalkyl optionally substituted with one or more hydroxy, and R² is C₁-C₆ alkyl;
(ix) R¹ is 3- to 7-membered heterocycloalkyl optionally substituted with one or more hydroxy, and R² is halo;
(x) R¹ is C₁-C₆ alkyl optionally substituted with one or more oxo, and R² is methyl;
(xi) R¹ is C₁-C₆ alkyl optionally substituted with one or more C₁-C₆ alkoxy, and R² is C₁-C₆ alkyl;
(xii) R¹ is C₁-C₆ alkyl optionally substituted with one or more NR⁸R⁹, and R² is C₁-C₆ alkyl;
(xiii) R¹ is C₁-C₆ alkyl optionally substituted with one or more NR⁸R⁹, and R² is halo;
(xiv) R² is C₁-C₆ alkyl optionally substituted with one or more hydroxy, and R¹ is C₆-C₁₀ aryl;
(xv) R² is C₁-C₆ alkyl optionally substituted with one or more hydroxy, and R¹ is 5- to 10-membered heteroaryl;
(xvi) R² is C₁-C₆ alkyl optionally substituted with one or more hydroxy, and R¹ is SF₅.
(xvii) R² is C₁-C₆ alkyl optionally substituted with one or more hydroxy, and R¹ is S(O₂)C₁-C₆ alkyl;
(xviii) R² is C₁-C₆ alkyl optionally substituted with one or more hydroxy, and R¹ is halo;
(xix) R² is C₃-C₇ cycloalkyl optionally substituted with one or more hydroxy, and R¹ is C₁-C₆ alkyl;
(xx) R² is 3- to 7-membered heterocycloalkyl optionally substituted with one or more hydroxy, and R¹ is C₁-C₆ alkyl;
(xxi) R² is 3- to 7-membered heterocycloalkyl optionally substituted with one or more hydroxy, and R¹ is halo;
(xxii) R² is C₁-C₆ alkyl optionally substituted with one or more oxo, and R¹ is methyl;
(xxiii) R² is C₁-C₆ alkyl optionally substituted with one or more C₁-C₆ alkoxy, and R¹ is C₁-C₆ alkyl;
(xxiv) R² is C₁-C₆ alkyl optionally substituted with one or more NR⁸R⁹, and R¹ is C₁-C₆ alkyl;
or
(xxv) R² is C₁-C₆ alkyl optionally substituted with one or more NR⁸R⁹, and R¹ is halo.

In some embodiments, of the compound of formula AA,

the substituted ring A is

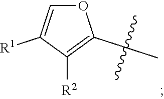

and R¹ and R² are one of the following combinations:
(i) R¹ is 1-hydroxy-2-methylpropan-2-yl, and R² is methyl;
(ii) R¹ is 2-hydroxy-2-propyl and R² is methyl;
(iii) R¹ is 2-hydroxy-2-propyl and R² is isopropyl;
(iv) R¹ is 2-hydroxy-2-propyl and R² is 2-hydroxy-2-propyl;
(v) R¹ is 2-hydroxy-2-propyl and R² is 1-hydroxyethyl;
(vi) R¹ is hydroxymethyl and R² is methyl;
(vii) R¹ is 1-hydroxyethyl and R² is methyl;
(viii) R¹ is 2-hydroxyethyl and R² is methyl;
(ix) R¹ is 1-hydroxy-2-propyl and R² is methyl;
(x) R¹ is 2-hydroxy-2-propyl and R² is phenyl;
(xi) R¹ is 2-hydroxy-2-propyl and R² is pyridyl;
(xii) R¹ is 2-hydroxy-2-propyl and R² is pyrazolyl;
(xiii) R¹ is 2-hydroxy-2-propyl, and R² is S(O₂)CH₃;
(xiv) R¹ is 2-hydroxy-2-propyl and R² is chloro;
(xv) R¹ is 2-hydroxy-2-propyl and R² is fluoro;
(xvi) R¹ is 1-hydroxy-1-cyclopropyl, and R² is methyl;
(xvii) R¹ is 1-hydroxy-1-cyclobutyl, and R² is methyl;
(xviii) R¹ is 1-hydroxy-1-cyclopentyl, and R² is methyl;
(xix) R¹ is 1-hydroxy-1-cyclohexyl, and R² is methyl;
(xx) R¹ is morpholinyl, and R² is methyl;
(xxi) R¹ is 1,3-dioxolan-2-yl, and R² is methyl;
(xxii) R¹ is 1,3-dioxolan-2-yl, and R² is fluoro;
(xxiii) R¹ is 1,3-dioxolan-2-yl, and R² is chloro;
(xxiv) R¹ is COCH₃, and R² is methyl;
(xxv) R¹ is 2-methoxy-2-propyl, and R² is methyl;
(xxvi) R¹ is (dimethylamino)methyl, and R² is methyl;
(xxvii) R² is 1-hydroxy-2-methylpropan-2-yl, and R¹ is methyl;
(xxviii) R² is 2-hydroxy-2-propyl and R¹ is methyl;
(xxix) R² is 2-hydroxy-2-propyl and R¹ is isopropyl;
(xxx) R² is 2-hydroxy-2-propyl and R¹ is 1-hydroxyethyl;
(xxxi) R² is hydroxymethyl and R¹ is methyl;
(xxxii) R² is 1-hydroxyethyl and R¹ is methyl;
(xxxiii) R² is 2-hydroxyethyl and R¹ is methyl;
(xxxiv) R² is 1-hydroxy-2-propyl and R¹ is methyl;
(xxxv) R² is 2-hydroxy-2-propyl and R¹ is phenyl;
(xxxvi) R² is C₁-C₆ alkyl optionally substituted with one or more hydroxy, and R¹ is 5- to 10-membered heteroaryl;
(xxxvii) R² is 2-hydroxy-2-propyl and R¹ is pyridyl;
(xxxviii) R² is 2-hydroxy-2-propyl and R¹ is pyrazolyl;
(xxxix) R² is C₁-C₆ alkyl optionally substituted with one or more hydroxy, and R¹ is S(O₂)CH₃;
(xl) R² is 2-hydroxy-2-propyl and R¹ is chloro;
(xli) R² is 2-hydroxy-2-propyl and R¹ is fluoro;
(xlii) R² is C₃-C₇ cycloalkyl optionally substituted with one or more hydroxy, and R¹ is C₁-C₆ alkyl;
(xliii) R² is 1-hydroxy-1-cyclopropyl, and R¹ is methyl;
(xliv) R² is 1-hydroxy-1-cyclobutyl, and R¹ is methyl;
(xlv) R² is 1-hydroxy-1-cyclopentyl, and R¹ is methyl;
(xlvi) R² is 1-hydroxy-1-cyclohexyl, and R¹ is methyl;
(xlvii) R² is morpholinyl, and R¹ is methyl;
(xlviii) R² is 1,3-dioxolan-2-yl, and R¹ is methyl;
(xlix) R² is 1,3-dioxolan-2-yl, and R¹ is fluoro;
(l) R² is 1,3-dioxolan-2-yl, and R¹ is chloro;
(li) R² is C₁-C₆ alkyl optionally substituted with one or more oxo, and R¹ is methyl;
(lii) R² is COCH₃, and R¹ is methyl;
(liii) R² is 2-methoxy-2-propyl, and R¹ is methyl;
or
(liv) R² is (dimethylamino)methyl, and R¹ is methyl.

In some embodiments, of the compound of formula AA,

the substituted ring A is

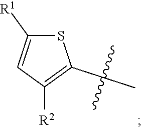

and R¹ and R² are one of the following combinations:
(i) R¹ is C₁-C₆ alkyl optionally substituted with one or more hydroxy, and R² is C₁-C₆ alkyl optionally substituted with one or more hydroxy;
(ii) R¹ is C₁-C₆ alkyl optionally substituted with one or more hydroxy, and R² is C₆-C₁₀ aryl;
(iii) R¹ is C₁-C₆ alkyl optionally substituted with one or more hydroxy, and R² is 5- to 10-membered heteroaryl;
(iv) R¹ is C₁-C₆ alkyl optionally substituted with one or more hydroxy, and R² is SF₅;
(v) R¹ is C₁-C₆ alkyl optionally substituted with one or more hydroxy, and R² is S(O₂)C₁-C₆ alkyl;
(vi) R¹ is C₁-C₆ alkyl optionally substituted with one or more hydroxy, and R² is halo;
(vii) R¹ is C₃-C₇ cycloalkyl optionally substituted with one or more hydroxy, and R² is C₁-C₆ alkyl;
(viii) R¹ is 3- to 7-membered heterocycloalkyl optionally substituted with one or more hydroxy, and R² is C₁-C₆ alkyl;
(ix) R¹ is 3- to 7-membered heterocycloalkyl optionally substituted with one or more hydroxy, and R² is halo;
(x) R¹ is C₁-C₆ alkyl optionally substituted with one or more oxo, and R² is methyl;
(xi) R¹ is C₁-C₆ alkyl optionally substituted with one or more C₁-C₆ alkoxy, and R² is C₁-C₆ alkyl;
(xii) R¹ is C₁-C₆ alkyl optionally substituted with one or more NR⁸R⁹, and R² is C₁-C₆ alkyl;
(xiii) R¹ is C₁-C₆ alkyl optionally substituted with one or more NR⁸R⁹, and R² is halo;
(xiv) R² is C₁-C₆ alkyl optionally substituted with one or more hydroxy, and R¹ is C₆-C₁₀ aryl;
(xv) R² is C₁-C₆ alkyl optionally substituted with one or more hydroxy, and R¹ is 5- to 10-membered heteroaryl;
(xvi) R² is C₁-C₆ alkyl optionally substituted with one or more hydroxy, and R¹ is SF₅.
(xvii) R² is C₁-C₆ alkyl optionally substituted with one or more hydroxy, and R¹ is S(O₂)C₁-C₆ alkyl;
(xviii) R² is C₁-C₆ alkyl optionally substituted with one or more hydroxy, and R¹ is halo;
(xix) R² is C₃-C₇ cycloalkyl optionally substituted with one or more hydroxy, and R¹ is C₁-C₆ alkyl;
(xx) R² is 3- to 7-membered heterocycloalkyl optionally substituted with one or more hydroxy, and R¹ is C₁-C₆ alkyl;
(xxi) R² is 3- to 7-membered heterocycloalkyl optionally substituted with one or more hydroxy, and R¹ is halo;
(xxii) R² is C₁-C₆ alkyl optionally substituted with one or more oxo, and R¹ is methyl;
(xxiii) R² is C₁-C₆ alkyl optionally substituted with one or more C₁-C₆ alkoxy, and R¹ is C₁-C₆ alkyl;
(xxiv) R² is C₁-C₆ alkyl optionally substituted with one or more NR⁸R⁹, and R¹ is C₁-C₆ alkyl;
or
(xxv) R² is C₁-C₆ alkyl optionally substituted with one or more NR⁸R⁹, and R¹ is halo.

In some embodiments, of the compound of formula AA,

the substituted ring A is

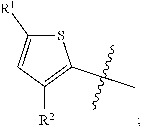

and R¹ and R² are one of the following combinations:
(i) R¹ is 1-hydroxy-2-methylpropan-2-yl, and R² is methyl;
(ii) R¹ is 2-hydroxy-2-propyl and R² is methyl;
(iii) R¹ is 2-hydroxy-2-propyl and R² is isopropyl;
(iv) R¹ is 2-hydroxy-2-propyl and R² is 2-hydroxy-2-propyl;
(v) R¹ is 2-hydroxy-2-propyl and R² is 1-hydroxyethyl;
(vi) R¹ is hydroxymethyl and R² is methyl;
(vii) R¹ is 1-hydroxyethyl and R² is methyl;
(viii) R¹ is 2-hydroxyethyl and R² is methyl;
(ix) R¹ is 1-hydroxy-2-propyl and R² is methyl;
(x) R¹ is 2-hydroxy-2-propyl and R² is phenyl;
(xi) R¹ is 2-hydroxy-2-propyl and R² is pyridyl;
(xii) R¹ is 2-hydroxy-2-propyl and R² is pyrazolyl;
(xiii) R¹ is 2-hydroxy-2-propyl, and R² is S(O₂)CH₃;
(xiv) R¹ is 2-hydroxy-2-propyl and R² is chloro;
(xv) R¹ is 2-hydroxy-2-propyl and R² is fluoro;
(xvi) R¹ is 1-hydroxy-1-cyclopropyl, and R² is methyl;
(xvii) R¹ is 1-hydroxy-1-cyclobutyl, and R² is methyl;
(xviii) R¹ is 1-hydroxy-1-cyclopentyl, and R² is methyl;
(xix) R¹ is 1-hydroxy-1-cyclohexyl, and R² is methyl;
(xx) R¹ is morpholinyl, and R² is methyl;
(xxi) R¹ is 1,3-dioxolan-2-yl, and R² is methyl;
(xxii) R¹ is 1,3-dioxolan-2-yl, and R² is fluoro;
(xxiii) R¹ is 1,3-dioxolan-2-yl, and R² is chloro;
(xxiv) R¹ is COCH₃, and R² is methyl;
(xxv) R¹ is 2-methoxy-2-propyl, and R² is methyl;
(xxvi) R¹ is (dimethylamino)methyl, and R² is methyl;
(xxvii) R² is 1-hydroxy-2-methylpropan-2-yl, and R¹ is methyl;
(xxviii) R² is 2-hydroxy-2-propyl and R¹ is methyl;
(xxix) R² is 2-hydroxy-2-propyl and R¹ is isopropyl;
(xxx) R² is 2-hydroxy-2-propyl and R¹ is 1-hydroxyethyl;
(xxxi) R² is hydroxymethyl and R¹ is methyl;
(xxxii) R² is 1-hydroxyethyl and R¹ is methyl;
(xxxiii) R² is 2-hydroxyethyl and R¹ is methyl;
(xxxiv) R² is 1-hydroxy-2-propyl and R¹ is methyl;
(xxxv) R² is 2-hydroxy-2-propyl and R¹ is phenyl;
(xxxvi) R² is C₁-C₆ alkyl optionally substituted with one or more hydroxy, and R¹ is 5- to 10-membered heteroaryl;
(xxxvii) R² is 2-hydroxy-2-propyl and R¹ is pyridyl;
(xxxviii) R² is 2-hydroxy-2-propyl and R¹ is pyrazolyl;
(xxxix) R² is C₁-C₆ alkyl optionally substituted with one or more hydroxy, and R¹ is S(O₂)CH₃;
(xl) R² is 2-hydroxy-2-propyl and R¹ is chloro;
(xli) R² is 2-hydroxy-2-propyl and R¹ is fluoro;
(xlii) R² is C₃-C₇ cycloalkyl optionally substituted with one or more hydroxy, and R¹ is C₁-C₆ alkyl;
(xliii) R² is 1-hydroxy-1-cyclopropyl, and R¹ is methyl;
(xliv) R² is 1-hydroxy-1-cyclobutyl, and R¹ is methyl;
(xlv) R² is 1-hydroxy-1-cyclopentyl, and R¹ is methyl;
(xlvi) R² is 1-hydroxy-1-cyclohexyl, and R¹ is methyl;
(xlvii) R² is morpholinyl, and R¹ is methyl;
(xlviii) R² is 1,3-dioxolan-2-yl, and R¹ is methyl;
(xlix) R² is 1,3-dioxolan-2-yl, and R¹ is fluoro;
(l) R² is 1,3-dioxolan-2-yl, and R¹ is chloro;
(li) R² is C₁-C₆ alkyl optionally substituted with one or more oxo, and R¹ is methyl;
(lii) R² is COCH₃, and R¹ is methyl;
(liii) R² is 2-methoxy-2-propyl, and R¹ is methyl;
or
(liv) R² is (dimethylamino)methyl, and R¹ is methyl.

In some embodiments, of the compound of formula AA,

the substituted ring A is

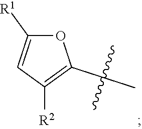

and R¹ and R² are one of the following combinations:
(i) R¹ is C₁-C₆ alkyl optionally substituted with one or more hydroxy, and R² is C₁-C₆ alkyl optionally substituted with one or more hydroxy;
(ii) R¹ is C₁-C₆ alkyl optionally substituted with one or more hydroxy, and R² is C₆-C₁₀ aryl;
(iii) R¹ is C₁-C₆ alkyl optionally substituted with one or more hydroxy, and R² is 5- to 10-membered heteroaryl;
(iv) R¹ is C₁-C₆ alkyl optionally substituted with one or more hydroxy, and R² is SF₅;
(v) R¹ is C₁-C₆ alkyl optionally substituted with one or more hydroxy, and R² is S(O₂)C₁-C₆ alkyl;
(vi) R¹ is C₁-C₆ alkyl optionally substituted with one or more hydroxy, and R² is halo;
(vii) R¹ is C₃-C₇ cycloalkyl optionally substituted with one or more hydroxy, and R² is C₁-C₆ alkyl;
(viii) R¹ is 3- to 7-membered heterocycloalkyl optionally substituted with one or more hydroxy, and R² is C₁-C₆ alkyl;
(ix) R¹ is 3- to 7-membered heterocycloalkyl optionally substituted with one or more hydroxy, and R² is halo;
(x) R¹ is C₁-C₆ alkyl optionally substituted with one or more oxo, and R² is methyl;
(xi) R¹ is C₁-C₆ alkyl optionally substituted with one or more C₁-C₆ alkoxy, and R² is C₁-C₆ alkyl;
(xii) R¹ is C₁-C₆ alkyl optionally substituted with one or more NR⁸R⁹, and R² is C₁-C₆ alkyl;
(xiii) R¹ is C₁-C₆ alkyl optionally substituted with one or more NR⁸R⁹, and R² is halo;
(xiv) R² is C₁-C₆ alkyl optionally substituted with one or more hydroxy, and R¹ is C₆-C₁₀ aryl;
(xv) R² is C₁-C₆ alkyl optionally substituted with one or more hydroxy, and R¹ is 5- to 10-membered heteroaryl;
(xvi) R² is C₁-C₆ alkyl optionally substituted with one or more hydroxy, and R¹ is SF₅.
(xvii) R² is C₁-C₆ alkyl optionally substituted with one or more hydroxy, and R¹ is S(O₂)C₁-C₆ alkyl;
(xviii) R² is C₁-C₆ alkyl optionally substituted with one or more hydroxy, and R¹ is halo;
(xix) R² is C₃-C₇ cycloalkyl optionally substituted with one or more hydroxy, and R¹ is C₁-C₆ alkyl;
(xx) R² is 3- to 7-membered heterocycloalkyl optionally substituted with one or more hydroxy, and R¹ is C₁-C₆ alkyl;
(xxi) R² is 3- to 7-membered heterocycloalkyl optionally substituted with one or more hydroxy, and R¹ is halo;
(xxii) R² is C₁-C₆ alkyl optionally substituted with one or more oxo, and R¹ is methyl;
(xxiii) R² is C₁-C₆ alkyl optionally substituted with one or more C₁-C₆ alkoxy, and R¹ is C₁-C₆ alkyl;
(xxiv) R² is C₁-C₆ alkyl optionally substituted with one or more NR⁸R⁹, and R¹ is C₁-C₆ alkyl;
(xxv) or
(xxvi) R² is C₁-C₆ alkyl optionally substituted with one or more NR⁸R⁹, and R¹ is halo.

In some embodiments, of the compound of formula AA,

the substituted ring A is

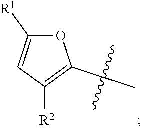

and R¹ and R² are one of the following combinations:
(i) R¹ is 1-hydroxy-2-methylpropan-2-yl, and R² is methyl;
(ii) R¹ is 2-hydroxy-2-propyl and R² is methyl;
(iii) R¹ is 2-hydroxy-2-propyl and R² is isopropyl;
(iv) R¹ is 2-hydroxy-2-propyl and R² is 2-hydroxy-2-propyl;
(v) R¹ is 2-hydroxy-2-propyl and R² is 1-hydroxyethyl;
(vi) R¹ is hydroxymethyl and R² is methyl;
(vii) R¹ is 1-hydroxyethyl and R² is methyl;
(viii) R¹ is 2-hydroxyethyl and R² is methyl;
(ix) R¹ is 1-hydroxy-2-propyl and R² is methyl;
(x) R¹ is 2-hydroxy-2-propyl and R² is phenyl;
(xi) R¹ is 2-hydroxy-2-propyl and R² is pyridyl;
(xii) R¹ is 2-hydroxy-2-propyl and R² is pyrazolyl;
(xiii) R¹ is 2-hydroxy-2-propyl, and R² is S(O₂)CH₃;
(xiv) R¹ is 2-hydroxy-2-propyl and R² is chloro;
(xv) R¹ is 2-hydroxy-2-propyl and R² is fluoro;
(xvi) R¹ is 1-hydroxy-1-cyclopropyl, and R² is methyl;
(xvii) R¹ is 1-hydroxy-1-cyclobutyl, and R² is methyl;
(xviii) R¹ is 1-hydroxy-1-cyclopentyl, and R² is methyl;
(xix) R¹ is 1-hydroxy-1-cyclohexyl, and R² is methyl;
(xx) R¹ is morpholinyl, and R² is methyl;
(xxi) R¹ is 1,3-dioxolan-2-yl, and R² is methyl;
(xxii) R¹ is 1,3-dioxolan-2-yl, and R² is fluoro;
(xxiii) R¹ is 1,3-dioxolan-2-yl, and R² is chloro;
(xxiv) R¹ is COCH₃, and R² is methyl;
(xxv) R¹ is 2-methoxy-2-propyl, and R² is methyl;
(xxvi) R¹ is (dimethylamino)methyl, and R² is methyl;
(xxvii) R² is 1-hydroxy-2-methylpropan-2-yl, and R¹ is methyl;
(xxviii) R² is 2-hydroxy-2-propyl and R¹ is methyl;
(xxix) R² is 2-hydroxy-2-propyl and R¹ is isopropyl;
(xxx) R² is 2-hydroxy-2-propyl and R¹ is 1-hydroxyethyl;
(xxxi) R² is hydroxymethyl and R¹ is methyl;
(xxxii) R² is 1-hydroxyethyl and R¹ is methyl;
(xxxiii) R² is 2-hydroxyethyl and R¹ is methyl;
(xxxiv) R² is 1-hydroxy-2-propyl and R¹ is methyl;
(xxxv) R² is 2-hydroxy-2-propyl and R¹ is phenyl;
(xxxvi) R² is C₁-C₆ alkyl optionally substituted with one or more hydroxy, and R¹ is 5- to 10-membered heteroaryl;
(xxxvii) R² is 2-hydroxy-2-propyl and R¹ is pyridyl;
(xxxviii) R² is 2-hydroxy-2-propyl and R¹ is pyrazolyl;
(xxxix) R² is C₁-C₆ alkyl optionally substituted with one or more hydroxy, and R¹ is S(O₂)CH₃;
(xl) R² is 2-hydroxy-2-propyl and R¹ is chloro;
(xli) R² is 2-hydroxy-2-propyl and R¹ is fluoro;
(xlii) R² is C₃-C₇ cycloalkyl optionally substituted with one or more hydroxy, and R¹ is C₁-C₆ alkyl;
(xliii) R² is 1-hydroxy-1-cyclopropyl, and R¹ is methyl;
(xliv) R² is 1-hydroxy-1-cyclobutyl, and R¹ is methyl;
(xlv) R² is 1-hydroxy-1-cyclopentyl, and R¹ is methyl;
(xlvi) R² is 1-hydroxy-1-cyclohexyl, and R¹ is methyl;
(xlvii) R² is morpholinyl, and R¹ is methyl;
(xlviii) R² is 1,3-dioxolan-2-yl, and R¹ is methyl;
(xlix) R² is 1,3-dioxolan-2-yl, and R¹ is fluoro;
(l) R² is 1,3-dioxolan-2-yl, and R¹ is chloro;
(li) R² is C₁-C₆ alkyl optionally substituted with one or more oxo, and R¹ is methyl;
(lii) R² is COCH₃, and R¹ is methyl;
(liii) R² is 2-methoxy-2-propyl, and R¹ is methyl;
or
(liv) R² is (dimethylamino)methyl, and R¹ is methyl.

In some embodiments, of the compound of formula AA,

the substituted ring A is

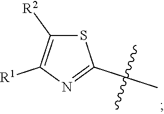

and R¹ and R² are one of the following combinations:
(i) R¹ is C₁-C₆ alkyl optionally substituted with one or more hydroxy, and R² is C₁-C₆ alkyl optionally substituted with one or more hydroxy;
(ii) R¹ is C₁-C₆ alkyl optionally substituted with one or more hydroxy, and R² is C₆-C₁₀ aryl;
(iii) R¹ is C₁-C₆ alkyl optionally substituted with one or more hydroxy, and R² is 5- to 10-membered heteroaryl;
(iv) R¹ is C₁-C₆ alkyl optionally substituted with one or more hydroxy, and R² is SF₅;
(v) R¹ is C₁-C₆ alkyl optionally substituted with one or more hydroxy, and R² is S(O₂)C₁-C₆ alkyl;
(vi) R¹ is C₁-C₆ alkyl optionally substituted with one or more hydroxy, and R² is halo;
(vii) R¹ is C₃-C₇ cycloalkyl optionally substituted with one or more hydroxy, and R² is C₁-C₆ alkyl;
(viii) R¹ is 3- to 7-membered heterocycloalkyl optionally substituted with one or more hydroxy, and R² is C₁-C₆ alkyl;
(ix) R¹ is 3- to 7-membered heterocycloalkyl optionally substituted with one or more hydroxy, and R² is halo;
(x) R¹ is C₁-C₆ alkyl optionally substituted with one or more oxo, and R² is methyl;
(xi) R¹ is C₁-C₆ alkyl optionally substituted with one or more C₁-C₆ alkoxy, and R² is C₁-C₆ alkyl;
(xii) R¹ is C₁-C₆ alkyl optionally substituted with one or more NR⁸R⁹, and R² is C₁-C₆ alkyl;
(xiii) R¹ is C₁-C₆ alkyl optionally substituted with one or more NR⁸R⁹, and R² is halo;
(xiv) R² is C₁-C₆ alkyl optionally substituted with one or more hydroxy, and R¹ is C₆-C₁₀ aryl;
(xv) R² is C₁-C₆ alkyl optionally substituted with one or more hydroxy, and R¹ is 5- to 10-membered heteroaryl;
(xvi) R² is C₁-C₆ alkyl optionally substituted with one or more hydroxy, and R¹ is SF₅;
(xvii) R² is C₁-C₆ alkyl optionally substituted with one or more hydroxy, and R¹ is S(O₂)C₁-C₆ alkyl;
(xviii) R² is C₁-C₆ alkyl optionally substituted with one or more hydroxy, and R¹ is halo;
(xix) R² is C₃-C₇ cycloalkyl optionally substituted with one or more hydroxy, and R¹ is C₁-C₆ alkyl;
(xx) R² is 3- to 7-membered heterocycloalkyl optionally substituted with one or more hydroxy, and R¹ is C₁-C₆ alkyl;
(xxi) R² is 3- to 7-membered heterocycloalkyl optionally substituted with one or more hydroxy, and R¹ is halo;
(xxii) R² is C₁-C₆ alkyl optionally substituted with one or more oxo, and R¹ is methyl;
(xxiii) R² is C₁-C₆ alkyl optionally substituted with one or more C₁-C₆ alkoxy, and R¹ is C₁-C₆ alkyl;
(xxiv) R² is C₁-C₆ alkyl optionally substituted with one or more NR⁸R⁹, and R¹ is C₁-C₆ alkyl;
or
(xxv) R² is C₁-C₆ alkyl optionally substituted with one or more NR⁸R⁹, and R¹ is halo.

In some embodiments, of the compound of formula AA,

the substituted ring A is

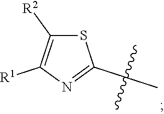

and R¹ and R² are one of the following combinations:
(i) R¹ is 1-hydroxy-2-methylpropan-2-yl, and R² is methyl;
(ii) R¹ is 2-hydroxy-2-propyl and R² is methyl;
(iii) R¹ is 2-hydroxy-2-propyl and R² is isopropyl;
(iv) R¹ is 2-hydroxy-2-propyl and R² is 2-hydroxy-2-propyl;
(v) R¹ is 2-hydroxy-2-propyl and R² is 1-hydroxyethyl;
(vi) R¹ is hydroxymethyl and R² is methyl;
(vii) R¹ is 1-hydroxyethyl and R² is methyl;
(viii) R¹ is 2-hydroxyethyl and R² is methyl;
(ix) R¹ is 1-hydroxy-2-propyl and R² is methyl;
(x) R¹ is 2-hydroxy-2-propyl and R² is phenyl;
(xi) R¹ is 2-hydroxy-2-propyl and R² is pyridyl;
(xii) R¹ is 2-hydroxy-2-propyl and R² is pyrazolyl;
(xiii) R¹ is 2-hydroxy-2-propyl, and R² is S(O₂)CH₃;
(xiv) R¹ is 2-hydroxy-2-propyl and R² is chloro;
(xv) R¹ is 2-hydroxy-2-propyl and R² is fluoro;
(xvi) R¹ is 1-hydroxy-1-cyclopropyl, and R² is methyl;
(xvii) R¹ is 1-hydroxy-1-cyclobutyl, and R² is methyl;
(xviii) R¹ is 1-hydroxy-1-cyclopentyl, and R² is methyl;
(xix) R¹ is 1-hydroxy-1-cyclohexyl, and R² is methyl;
(xx) R¹ is morpholinyl, and R² is methyl;
(xxi) R¹ is 1,3-dioxolan-2-yl, and R² is methyl;
(xxii) R¹ is 1,3-dioxolan-2-yl, and R² is fluoro;
(xxiii) R¹ is 1,3-dioxolan-2-yl, and R² is chloro;
(xxiv) R¹ is COCH₃, and R² is methyl;
(xxv) R¹ is 2-methoxy-2-propyl, and R² is methyl;
(xxvi) R¹ is (dimethylamino)methyl, and R² is methyl;
(xxvii) R² is 1-hydroxy-2-methylpropan-2-yl, and R¹ is methyl;
(xxviii) R² is 2-hydroxy-2-propyl and R¹ is methyl;
(xxix) R² is 2-hydroxy-2-propyl and R¹ is isopropyl;
(xxx) R² is 2-hydroxy-2-propyl and R¹ is 1-hydroxyethyl;
(xxxi) R² is hydroxymethyl and R¹ is methyl;
(xxxii) R² is 1-hydroxyethyl and R¹ is methyl;
(xxxiii) R² is 2-hydroxyethyl and R¹ is methyl;
(xxxiv) R² is 1-hydroxy-2-propyl and R¹ is methyl;
(xxxv) R² is 2-hydroxy-2-propyl and R¹ is phenyl;
(xxxvi) R² is C₁-C₆ alkyl optionally substituted with one or more hydroxy, and R¹ is 5- to 10-membered heteroaryl;
(xxxvii) R² is 2-hydroxy-2-propyl and R¹ is pyridyl;
(xxxviii) R² is 2-hydroxy-2-propyl and R¹ is pyrazolyl;
(xxxix) R² is C₁-C₆ alkyl optionally substituted with one or more hydroxy, and R¹ is S(O₂)CH₃;
(xl) R² is 2-hydroxy-2-propyl and R¹ is chloro;
(xli) R² is 2-hydroxy-2-propyl and R¹ is fluoro;
(xlii) R² is C₃-C₇ cycloalkyl optionally substituted with one or more hydroxy, and R¹ is C₁-C₆ alkyl;
(xliii) R² is 1-hydroxy-1-cyclopropyl, and R¹ is methyl;
(xliv) R² is 1-hydroxy-1-cyclobutyl, and R¹ is methyl;
(xlv) R² is 1-hydroxy-1-cyclopentyl, and R¹ is methyl;
(xlvi) R² is 1-hydroxy-1-cyclohexyl, and R¹ is methyl;
(xlvii) R² is morpholinyl, and R¹ is methyl;
(xlviii) R² is 1,3-dioxolan-2-yl, and R¹ is methyl;
(xlix) R² is 1,3-dioxolan-2-yl, and R¹ is fluoro;
(l) R² is 1,3-dioxolan-2-yl, and R¹ is chloro;
(li) R² is C₁-C₆ alkyl optionally substituted with one or more oxo, and R¹ is methyl;
(lii) R² is COCH₃, and R¹ is methyl;
(liii) R² is 2-methoxy-2-propyl, and R¹ is methyl;
or
(liv) R² is (dimethylamino)methyl, and R¹ is methyl.

In some embodiments, of the compound of formula AA,

the substituted ring A is

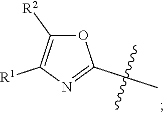

and R¹ and R² are one of the following combinations:
(i) R¹ is C₁-C₆ alkyl optionally substituted with one or more hydroxy, and R² is C₁-C₆ alkyl optionally substituted with one or more hydroxy;
(ii) R¹ is C₁-C₆ alkyl optionally substituted with one or more hydroxy, and R² is C₆-C₁₀ aryl;
(iii) R¹ is C₁-C₆ alkyl optionally substituted with one or more hydroxy, and R² is 5- to 10-membered heteroaryl;
(iv) R¹ is C₁-C₆ alkyl optionally substituted with one or more hydroxy, and R² is SF₅;
(v) R¹ is C₁-C₆ alkyl optionally substituted with one or more hydroxy, and R² is S(O₂)C₁-C₆ alkyl;
(vi) R¹ is C₁-C₆ alkyl optionally substituted with one or more hydroxy, and R² is halo;
(vii) R¹ is C₃-C₇ cycloalkyl optionally substituted with one or more hydroxy, and R² is C₁-C₆ alkyl;
(viii) R¹ is 3- to 7-membered heterocycloalkyl optionally substituted with one or more hydroxy, and R² is C₁-C₆ alkyl;
(ix) R¹ is 3- to 7-membered heterocycloalkyl optionally substituted with one or more hydroxy, and R² is halo;
(x) R¹ is C₁-C₆ alkyl optionally substituted with one or more oxo, and R² is methyl;
(xi) R¹ is C₁-C₆ alkyl optionally substituted with one or more C₁-C₆ alkoxy, and R² is C₁-C₆ alkyl;
(xii) R¹ is C₁-C₆ alkyl optionally substituted with one or more NR⁸R⁹, and R² is C₁-C₆ alkyl;
(xiii) R¹ is C₁-C₆ alkyl optionally substituted with one or more NR⁸R⁹, and R² is halo;
(xiv) R² is C₁-C₆ alkyl optionally substituted with one or more hydroxy, and R¹ is C₆-C₁₀ aryl;
(xv) R² is C₁-C₆ alkyl optionally substituted with one or more hydroxy, and R¹ is 5- to 10-membered heteroaryl;
(xvi) R² is C₁-C₆ alkyl optionally substituted with one or more hydroxy, and R¹ is SF₅.
(xvii) R² is C₁-C₆ alkyl optionally substituted with one or more hydroxy, and R¹ is S(O₂)C₁-C₆ alkyl;
(xviii) R² is C₁-C₆ alkyl optionally substituted with one or more hydroxy, and R¹ is halo;
(xix) R² is C₃-C₇ cycloalkyl optionally substituted with one or more hydroxy, and R¹ is C₁-C₆ alkyl;
(xx) R² is 3- to 7-membered heterocycloalkyl optionally substituted with one or more hydroxy, and R¹ is C₁-C₆ alkyl;
(xxi) R² is 3- to 7-membered heterocycloalkyl optionally substituted with one or more hydroxy, and R¹ is halo;
(xxii) R² is C₁-C₆ alkyl optionally substituted with one or more oxo, and R¹ is methyl;
(xxiii) R² is C₁-C₆ alkyl optionally substituted with one or more C₁-C₆ alkoxy, and R¹ is C₁-C₆ alkyl;
(xxiv) R² is C₁-C₆ alkyl optionally substituted with one or more NR⁸R⁹, and R¹ is C₁-C₆ alkyl;
or
(xxv) R² is C₁-C₆ alkyl optionally substituted with one or more NR⁸R⁹, and R¹ is halo.

In some embodiments, of the compound of formula AA,

the substituted ring A is

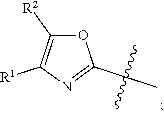

and R¹ and R² are one of the following combinations:
(i) R¹ is 1-hydroxy-2-methylpropan-2-yl, and R² is methyl;
(ii) R¹ is 2-hydroxy-2-propyl and R² is methyl;
(iii) R¹ is 2-hydroxy-2-propyl and R² is isopropyl;
(iv) R¹ is 2-hydroxy-2-propyl and R² is 2-hydroxy-2-propyl;
(v) R¹ is 2-hydroxy-2-propyl and R² is 1-hydroxyethyl;
(vi) R¹ is hydroxymethyl and R² is methyl;
(vii) R¹ is 1-hydroxyethyl and R² is methyl;
(viii) R¹ is 2-hydroxyethyl and R² is methyl;
(ix) R¹ is 1-hydroxy-2-propyl and R² is methyl;
(x) R¹ is 2-hydroxy-2-propyl and R² is phenyl;
(xi) R¹ is 2-hydroxy-2-propyl and R² is pyridyl;
(xii) R¹ is 2-hydroxy-2-propyl and R² is pyrazolyl;
(xiii) R¹ is 2-hydroxy-2-propyl, and R² is S(O₂)CH₃;
(xiv) R¹ is 2-hydroxy-2-propyl and R² is chloro;
(xv) R¹ is 2-hydroxy-2-propyl and R² is fluoro;
(xvi) R¹ is 1-hydroxy-1-cyclopropyl, and R² is methyl;
(xvii) R¹ is 1-hydroxy-1-cyclobutyl, and R² is methyl;
(xviii) R¹ is 1-hydroxy-1-cyclopentyl, and R² is methyl;
(xix) R¹ is 1-hydroxy-1-cyclohexyl, and R² is methyl;
(xx) R¹ is morpholinyl, and R² is methyl;
(xxi) R¹ is 1,3-dioxolan-2-yl, and R² is methyl;
(xxii) R¹ is 1,3-dioxolan-2-yl, and R² is fluoro;
(xxiii) R¹ is 1,3-dioxolan-2-yl, and R² is chloro;
(xxiv) R¹ is COCH₃, and R² is methyl;
(xxv) R¹ is 2-methoxy-2-propyl, and R² is methyl;
(xxvi) R¹ is (dimethylamino)methyl, and R² is methyl;
(xxvii) R² is 1-hydroxy-2-methylpropan-2-yl, and R¹ is methyl;
(xxviii) R² is 2-hydroxy-2-propyl and R¹ is methyl;
(xxix) R² is 2-hydroxy-2-propyl and R¹ is isopropyl;
(xxx) R² is 2-hydroxy-2-propyl and R¹ is 1-hydroxyethyl;
(xxxi) R² is hydroxymethyl and R¹ is methyl;
(xxxii) R² is 1-hydroxyethyl and R¹ is methyl;
(xxxiii) R² is 2-hydroxyethyl and R¹ is methyl;
(xxxiv) R² is 1-hydroxy-2-propyl and R¹ is methyl;
(xxxv) R² is 2-hydroxy-2-propyl and R¹ is phenyl;
(xxxvi) R² is C₁-C₆ alkyl optionally substituted with one or more hydroxy, and R¹ is 5- to 10-membered heteroaryl;
(xxxvii) R² is 2-hydroxy-2-propyl and R¹ is pyridyl;
(xxxviii) R² is 2-hydroxy-2-propyl and R¹ is pyrazolyl;
(xxxix) R² is C₁-C₆ alkyl optionally substituted with one or more hydroxy, and R¹ is S(O₂)CH₃;
(xl) R² is 2-hydroxy-2-propyl and R¹ is chloro;
(xli) R² is 2-hydroxy-2-propyl and R¹ is fluoro;
(xlii) R² is C₃-C₇ cycloalkyl optionally substituted with one or more hydroxy, and R¹ is C₁-C₆ alkyl;
(xliii) R² is 1-hydroxy-1-cyclopropyl, and R¹ is methyl;
(xliv) R² is 1-hydroxy-1-cyclobutyl, and R¹ is methyl;
(xlv) R² is 1-hydroxy-1-cyclopentyl, and R¹ is methyl;
(xlvi) R² is 1-hydroxy-1-cyclohexyl, and R¹ is methyl;
(xlvii) R² is morpholinyl, and R¹ is methyl;
(xlviii) R² is 1,3-dioxolan-2-yl, and R¹ is methyl;
(xlix) R² is 1,3-dioxolan-2-yl, and R¹ is fluoro;
(l) R² is 1,3-dioxolan-2-yl, and R¹ is chloro;
(li) R² is C₁-C₆ alkyl optionally substituted with one or more oxo, and R¹ is methyl;
(lii) R² is COCH₃, and R¹ is methyl;
(liii) R² is 2-methoxy-2-propyl, and R⁸ is methyl;
or
(liv) R² is (dimethylamino)methyl, and R⁸ is methyl.

In some embodiments, of the compound of formula AA,

the substituted ring A is

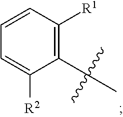

and R¹ and R² are one of the following combinations:
(i) R¹ is C₁-C₆ alkyl optionally substituted with one or more hydroxy, and R² is C₁-C₆ alkyl optionally substituted with one or more hydroxy;
(ii) R¹ is C₁-C₆ alkyl optionally substituted with one or more hydroxy, and R² is C₆-C₁₀ aryl;
(iii) R¹ is C₁-C₆ alkyl optionally substituted with one or more hydroxy, and R² is 5- to 10-membered heteroaryl;
(iv) R¹ is C₁-C₆ alkyl optionally substituted with one or more hydroxy, and R² is SF₅;
(v) R¹ is C₁-C₆ alkyl optionally substituted with one or more hydroxy, and R² is S(O₂)C₁-C₆ alkyl;
(vi) R¹ is C₁-C₆ alkyl optionally substituted with one or more hydroxy, and R² is halo;
(vii) R¹ is C₃-C₇ cycloalkyl optionally substituted with one or more hydroxy, and R² is C₁-C₆ alkyl;
(viii) R¹ is 3- to 7-membered heterocycloalkyl optionally substituted with one or more hydroxy, and R² is C₁-C₆ alkyl;
(ix) R¹ is 3- to 7-membered heterocycloalkyl optionally substituted with one or more hydroxy, and R² is halo;
(x) R¹ is C₁-C₆ alkyl optionally substituted with one or more oxo, and R² is methyl;
(xi) R¹ is C₁-C₆ alkyl optionally substituted with one or more C₁-C₆ alkoxy, and R² is C₁-C₆ alkyl;
(xii) R¹ is C₁-C₆ alkyl optionally substituted with one or more NR⁸R⁹, and R² is C₁-C₆ alkyl;
(xiii) R¹ is C₁-C₆ alkyl optionally substituted with one or more NR⁸R⁹, and R² is halo;
(xiv) R² is C₁-C₆ alkyl optionally substituted with one or more hydroxy, and R¹ is C₆-C₁₀ aryl;
(xv) R² is C₁-C₆ alkyl optionally substituted with one or more hydroxy, and R¹ is 5- to 10-membered heteroaryl;
(xvi) R² is C₁-C₆ alkyl optionally substituted with one or more hydroxy, and R¹ is SF₅.
(xvii) R² is C₁-C₆ alkyl optionally substituted with one or more hydroxy, and R¹ is S(O₂)C₁-C₆ alkyl;
(xviii) R² is C₁-C₆ alkyl optionally substituted with one or more hydroxy, and R¹ is halo;
(xix) R² is C₃-C₇ cycloalkyl optionally substituted with one or more hydroxy, and R¹ is C₁-C₆ alkyl;
(xx) R² is 3- to 7-membered heterocycloalkyl optionally substituted with one or more hydroxy, and R¹ is C₁-C₆ alkyl;
(xxi) R² is 3- to 7-membered heterocycloalkyl optionally substituted with one or more hydroxy, and R¹ is halo;
(xxii) R² is C₁-C₆ alkyl optionally substituted with one or more oxo, and R¹ is methyl;
(xxiii) R² is C₁-C₆ alkyl optionally substituted with one or more C₁-C₆ alkoxy, and R¹ is C₁-C₆ alkyl;
(xxiv) R² is C₁-C₆ alkyl optionally substituted with one or more NR⁸R⁹, and R¹ is C₁-C₆ alkyl;
or
(xxv) R² is C₁-C₆ alkyl optionally substituted with one or more NR⁸R⁹, and R¹ is halo.

In some embodiments, of the compound of formula AA,

the substituted ring A is

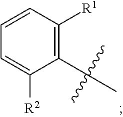

and R¹ and R² are one of the following combinations:
(i) R¹ is 1-hydroxy-2-methylpropan-2-yl, and R² is methyl;
(ii) R¹ is 2-hydroxy-2-propyl and R² is methyl;
(iii) R¹ is 2-hydroxy-2-propyl and R² is isopropyl;
(iv) R¹ is 2-hydroxy-2-propyl and R² is 2-hydroxy-2-propyl;
(v) R¹ is 2-hydroxy-2-propyl and R² is 1-hydroxyethyl;
(vi) R¹ is hydroxymethyl and R² is methyl;
(vii) R¹ is 1-hydroxyethyl and R² is methyl;
(viii) R¹ is 2-hydroxyethyl and R² is methyl;
(ix) R¹ is 1-hydroxy-2-propyl and R² is methyl;
(x) R¹ is 2-hydroxy-2-propyl and R² is phenyl;
(xi) R¹ is 2-hydroxy-2-propyl and R² is pyridyl;
(xii) R¹ is 2-hydroxy-2-propyl and R² is pyrazolyl;
(xiii) R¹ is 2-hydroxy-2-propyl, and R² is S(O₂)CH₃;
(xiv) R¹ is 2-hydroxy-2-propyl and R² is chloro;
(xv) R¹ is 2-hydroxy-2-propyl and R² is fluoro;
(xvi) R¹ is 1-hydroxy-1-cyclopropyl, and R² is methyl;
(xvii) R¹ is 1-hydroxy-1-cyclobutyl, and R² is methyl;
(xviii) R¹ is 1-hydroxy-1-cyclopentyl, and R² is methyl;
(xix) R¹ is 1-hydroxy-1-cyclohexyl, and R² is methyl;
(xx) R¹ is morpholinyl, and R² is methyl;
(xxi) R¹ is 1,3-dioxolan-2-yl, and R² is methyl;
(xxii) R¹ is 1,3-dioxolan-2-yl, and R² is fluoro;
(xxiii) R¹ is 1,3-dioxolan-2-yl, and R² is chloro;
(xxiv) R¹ is COCH₃, and R² is methyl;
(xxv) R¹ is 2-methoxy-2-propyl, and R² is methyl;
(xxvi) R¹ is (dimethylamino)methyl, and R² is methyl;
(xxvii) R² is 1-hydroxy-2-methylpropan-2-yl, and R¹ is methyl;
(xxviii) R² is 2-hydroxy-2-propyl and R¹ is methyl;
(xxix) R² is 2-hydroxy-2-propyl and R¹ is isopropyl;
(xxx) R² is 2-hydroxy-2-propyl and R¹ is 1-hydroxyethyl;
(xxxi) R² is hydroxymethyl and R¹ is methyl;
(xxxii) R² is 1-hydroxyethyl and R¹ is methyl;
(xxxiii) R² is 2-hydroxyethyl and R¹ is methyl;
(xxxiv) R² is 1-hydroxy-2-propyl and R¹ is methyl;
(xxxv) R² is 2-hydroxy-2-propyl and R¹ is phenyl;
(xxxvi) R² is C₁-C₆ alkyl optionally substituted with one or more hydroxy, and R¹ is 5- to 10-membered heteroaryl;
(xxxvii) R² is 2-hydroxy-2-propyl and R¹ is pyridyl;
(xxxviii) R² is 2-hydroxy-2-propyl and R¹ is pyrazolyl;
(xxxix) R² is C₁-C₆ alkyl optionally substituted with one or more hydroxy, and R¹ is S(O₂)CH₃;
(xl) R² is 2-hydroxy-2-propyl and R¹ is chloro;
(xli) R² is 2-hydroxy-2-propyl and R¹ is fluoro;
(xlii) R² is C₃-C₇ cycloalkyl optionally substituted with one or more hydroxy, and R¹ is C₁-C₆ alkyl;
(xliii) R² is 1-hydroxy-1-cyclopropyl, and R¹ is methyl;
(xliv) R² is 1-hydroxy-1-cyclobutyl, and R¹ is methyl;
(xlv) R² is 1-hydroxy-1-cyclopentyl, and R¹ is methyl;
(xlvi) R² is 1-hydroxy-1-cyclohexyl, and R¹ is methyl;
(xlvii) R² is morpholinyl, and R¹ is methyl;
(xlviii) R² is 1,3-dioxolan-2-yl, and R¹ is methyl;
(xlix) R² is 1,3-dioxolan-2-yl, and R¹ is fluoro;
(l) R² is 1,3-dioxolan-2-yl, and R¹ is chloro;
(li) R² is C₁-C₆ alkyl optionally substituted with one or more oxo, and R¹ is methyl;
(lii) R² is COCH₃, and R¹ is methyl;
(liii) R² is 2-methoxy-2-propyl, and R¹ is methyl;
or
(liv) R² is (dimethylamino)methyl, and R¹ is methyl.

In some embodiments, of the compound of formula AA,

the substituted ring A is

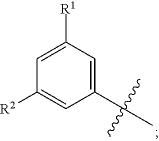

and R¹ and R² are one of the following combinations:
(i) R¹ is C₁-C₆ alkyl optionally substituted with one or more hydroxy, and R² is C₁-C₆ alkyl optionally substituted with one or more hydroxy;
(ii) R¹ is C₁-C₆ alkyl optionally substituted with one or more hydroxy, and R² is C₆-C₁₀ aryl;
(iii) R¹ is C₁-C₆ alkyl optionally substituted with one or more hydroxy, and R² is 5- to 10-membered heteroaryl;
(iv) R¹ is C₁-C₆ alkyl optionally substituted with one or more hydroxy, and R² is SF₅;
(v) R¹ is C₁-C₆ alkyl optionally substituted with one or more hydroxy, and R² is S(O₂)C₁-C₆ alkyl;
(vi) R¹ is C₁-C₆ alkyl optionally substituted with one or more hydroxy, and R² is halo;
(vii) R¹ is C₃-C₇ cycloalkyl optionally substituted with one or more hydroxy, and R² is C₁-C₆ alkyl;
(viii) R¹ is 3- to 7-membered heterocycloalkyl optionally substituted with one or more hydroxy, and R² is C₁-C₆ alkyl;
(ix) R¹ is 3- to 7-membered heterocycloalkyl optionally substituted with one or more hydroxy, and R² is halo;
(x) R¹ is C₁-C₆ alkyl optionally substituted with one or more oxo, and R² is methyl;
(xi) R¹ is C₁-C₆ alkyl optionally substituted with one or more C₁-C₆ alkoxy, and R² is C₁-C₆ alkyl;
(xii) R¹ is C₁-C₆ alkyl optionally substituted with one or more NR⁸R⁹, and R² is C₁-C₆ alkyl;
(xiii) R¹ is C₁-C₆ alkyl optionally substituted with one or more NR⁸R⁹, and R² is halo;
(xiv) R² is C₁-C₆ alkyl optionally substituted with one or more hydroxy, and R¹ is C₆-C₁₀ aryl;
(xv) R² is C₁-C₆ alkyl optionally substituted with one or more hydroxy, and R¹ is 5- to 10-membered heteroaryl;
(xvi) R² is C₁-C₆ alkyl optionally substituted with one or more hydroxy, and R¹ is SF₅.
(xvii) R² is C₁-C₆ alkyl optionally substituted with one or more hydroxy, and R¹ is S(O₂)C₁-C₆ alkyl;
(xviii) R² is C₁-C₆ alkyl optionally substituted with one or more hydroxy, and R¹ is halo;
(xix) R² is C₃-C₇ cycloalkyl optionally substituted with one or more hydroxy, and R¹ is C₁-C₆ alkyl;
(xx) R² is 3- to 7-membered heterocycloalkyl optionally substituted with one or more hydroxy, and R¹ is C₁-C₆ alkyl;
(xxi) R² is 3- to 7-membered heterocycloalkyl optionally substituted with one or more hydroxy, and R¹ is halo;
(xxii) R² is C₁-C₆ alkyl optionally substituted with one or more oxo, and R¹ is methyl;
(xxiii) R² is C₁-C₆ alkyl optionally substituted with one or more C₁-C₆ alkoxy, and R¹ is C₁-C₆ alkyl;
(xxiv) R² is C₁-C₆ alkyl optionally substituted with one or more NR⁸R⁹, and R¹ is C₁-C₆ alkyl;
or
(xxv) R² is C₁-C₆ alkyl optionally substituted with one or more NR⁸R⁹, and R¹ is halo.

In some embodiments, of the compound of formula AA,

the substituted ring A is

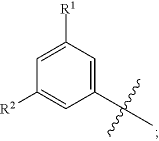

and R¹ and R² are one of the following combinations:
(i) R¹ is 1-hydroxy-2-methylpropan-2-yl, and R² is methyl;
(ii) R¹ is 2-hydroxy-2-propyl and R² is methyl;
(iii) R¹ is 2-hydroxy-2-propyl and R² is isopropyl;
(iv) R¹ is 2-hydroxy-2-propyl and R² is 2-hydroxy-2-propyl;
(v) R¹ is 2-hydroxy-2-propyl and R² is 1-hydroxyethyl;
(vi) R¹ is hydroxymethyl and R² is methyl;
(vii) R¹ is 1-hydroxyethyl and R² is methyl;
(viii) R¹ is 2-hydroxyethyl and R² is methyl;
(ix) R¹ is 1-hydroxy-2-propyl and R² is methyl;
(x) R¹ is 2-hydroxy-2-propyl and R² is phenyl;
(xi) R¹ is 2-hydroxy-2-propyl and R² is pyridyl;
(xii) R¹ is 2-hydroxy-2-propyl and R² is pyrazolyl;
(xiii) R¹ is 2-hydroxy-2-propyl, and R² is S(O₂)CH₃;
(xiv) R¹ is 2-hydroxy-2-propyl and R² is chloro;
(xv) R¹ is 2-hydroxy-2-propyl and R² is fluoro;
(xvi) R¹ is 1-hydroxy-1-cyclopropyl, and R² is methyl;
(xvii) R¹ is 1-hydroxy-1-cyclobutyl, and R² is methyl;
(xviii) R¹ is 1-hydroxy-1-cyclopentyl, and R² is methyl;
(xix) R¹ is 1-hydroxy-1-cyclohexyl, and R² is methyl;
(xx) R¹ is morpholinyl, and R² is methyl;
(xxi) R¹ is 1,3-dioxolan-2-yl, and R² is methyl;
(xxii) R¹ is 1,3-dioxolan-2-yl, and R² is fluoro;
(xxiii) R¹ is 1,3-dioxolan-2-yl, and R² is chloro;
(xxiv) R¹ is COCH₃, and R² is methyl;
(xxv) R¹ is 2-methoxy-2-propyl, and R² is methyl;
(xxvi) R¹ is (dimethylamino)methyl, and R² is methyl;
(xxvii) R² is 1-hydroxy-2-methylpropan-2-yl, and R¹ is methyl;
(xxviii) R² is 2-hydroxy-2-propyl and R¹ is methyl;
(xxix) R² is 2-hydroxy-2-propyl and R¹ is isopropyl;
(xxx) R² is 2-hydroxy-2-propyl and R¹ is 1-hydroxyethyl;
(xxxi) R² is hydroxymethyl and R¹ is methyl;
(xxxii) R² is 1-hydroxyethyl and R¹ is methyl;
(xxxiii) R² is 2-hydroxyethyl and R¹ is methyl;
(xxxiv) R² is 1-hydroxy-2-propyl and R¹ is methyl;
(xxxv) R² is 2-hydroxy-2-propyl and R¹ is phenyl;
(xxxvi) R² is C₁-C₆ alkyl optionally substituted with one or more hydroxy, and R¹ is 5- to 10-membered heteroaryl;
(xxxvii) R² is 2-hydroxy-2-propyl and R¹ is pyridyl;
(xxxviii) R² is 2-hydroxy-2-propyl and R¹ is pyrazolyl;
(xxxix) R² is C₁-C₆ alkyl optionally substituted with one or more hydroxy, and R¹ is S(O₂)CH₃;
(xl) R² is 2-hydroxy-2-propyl and R¹ is chloro;
(xli) R² is 2-hydroxy-2-propyl and R¹ is fluoro;
(xlii) R² is C₃-C₇ cycloalkyl optionally substituted with one or more hydroxy, and R¹ is C₁-C₆ alkyl;
(xliii) R² is 1-hydroxy-1-cyclopropyl, and R¹ is methyl;
(xliv) R² is 1-hydroxy-1-cyclobutyl, and R¹ is methyl;
(xlv) R² is 1-hydroxy-1-cyclopentyl, and R¹ is methyl;
(xlvi) R² is 1-hydroxy-1-cyclohexyl, and R¹ is methyl;
(xlvii) R² is morpholinyl, and R¹ is methyl;
(xlviii) R² is 1,3-dioxolan-2-yl, and R¹ is methyl;
(xlix) R² is 1,3-dioxolan-2-yl, and R¹ is fluoro;
(l) R² is 1,3-dioxolan-2-yl, and R¹ is chloro;
(li) R² is C₁-C₆ alkyl optionally substituted with one or more oxo, and R¹ is methyl;
(lii) R² is COCH₃, and R¹ is methyl;
(liii) R² is 2-methoxy-2-propyl, and R¹ is methyl;
or
(liv) R² is (dimethylamino)methyl, and R¹ is methyl.

In some embodiments, of the compound of formula AA,

the substituted ring A is

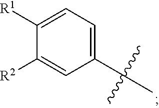

and R¹ and R² are one of the following combinations:
(i) R¹ is C₁-C₆ alkyl optionally substituted with one or more hydroxy, and R² is C₁-C₆ alkyl optionally substituted with one or more hydroxy;
(ii) R¹ is C₁-C₆ alkyl optionally substituted with one or more hydroxy, and R² is C₆-C₁₀ aryl;
(iii) R¹ is C₁-C₆ alkyl optionally substituted with one or more hydroxy, and R² is 5- to 10-membered heteroaryl;
(iv) R¹ is C₁-C₆ alkyl optionally substituted with one or more hydroxy, and R² is SF₅;
(v) R¹ is C₁-C₆ alkyl optionally substituted with one or more hydroxy, and R² is S(O₂)C₁-C₆ alkyl;
(vi) R¹ is C₁-C₆ alkyl optionally substituted with one or more hydroxy, and R² is halo;
(vii) R¹ is C₃-C₇ cycloalkyl optionally substituted with one or more hydroxy, and R² is C₁-C₆ alkyl;
(viii) R¹ is 3- to 7-membered heterocycloalkyl optionally substituted with one or more hydroxy, and R² is C₁-C₆ alkyl;
(ix) R¹ is 3- to 7-membered heterocycloalkyl optionally substituted with one or more hydroxy, and R² is halo;
(x) R¹ is C₁-C₆ alkyl optionally substituted with one or more oxo, and R² is methyl;
(xi) R¹ is C₁-C₆ alkyl optionally substituted with one or more C₁-C₆ alkoxy, and R² is C₁-C₆ alkyl;
(xii) R¹ is C₁-C₆ alkyl optionally substituted with one or more NR⁸R⁹, and R² is C₁-C₆ alkyl;
(xiii) R¹ is C₁-C₆ alkyl optionally substituted with one or more NR⁸R⁹, and R² is halo;
(xiv) R² is C₁-C₆ alkyl optionally substituted with one or more hydroxy, and R¹ is C₆-C₁₀ aryl;
(xv) R² is C₁-C₆ alkyl optionally substituted with one or more hydroxy, and R¹ is 5- to 10-membered heteroaryl;
(xvi) R² is C₁-C₆ alkyl optionally substituted with one or more hydroxy, and R¹ is SF₅.
(xvii) R² is C₁-C₆ alkyl optionally substituted with one or more hydroxy, and R¹ is S(O₂)C₁-C₆ alkyl;
(xviii) R² is C₁-C₆ alkyl optionally substituted with one or more hydroxy, and R¹ is halo;
(xix) R² is C₃-C₇ cycloalkyl optionally substituted with one or more hydroxy, and R¹ is C₁-C₆ alkyl;
(xx) R² is 3- to 7-membered heterocycloalkyl optionally substituted with one or more hydroxy, and R¹ is C₁-C₆ alkyl;
(xxi) R² is 3- to 7-membered heterocycloalkyl optionally substituted with one or more hydroxy, and R¹ is halo;
(xxii) R² is C₁-C₆ alkyl optionally substituted with one or more oxo, and R¹ is methyl;
(xxiii) R² is C₁-C₆ alkyl optionally substituted with one or more C₁-C₆ alkoxy, and R¹ is C₁-C₆ alkyl;
(xxiv) R² is C₁-C₆ alkyl optionally substituted with one or more NR⁸R⁹, and R¹ is C₁-C₆ alkyl;
or
(xxv) R² is C₁-C₆ alkyl optionally substituted with one or more NR⁸R⁹, and R¹ is halo.

In some embodiments, of the compound of formula AA,

the substituted ring A is

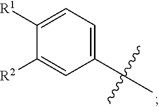

and R¹ and R² are one of the following combinations:
(i) R¹ is 1-hydroxy-2-methylpropan-2-yl, and R² is methyl;
(ii) R¹ is 2-hydroxy-2-propyl and R² is methyl;
(iii) R¹ is 2-hydroxy-2-propyl and R² is isopropyl;
(iv) R¹ is 2-hydroxy-2-propyl and R² is 2-hydroxy-2-propyl;
(v) R¹ is 2-hydroxy-2-propyl and R² is 1-hydroxyethyl;
(vi) R¹ is hydroxymethyl and R² is methyl;
(vii) R¹ is 1-hydroxyethyl and R² is methyl;
(viii) R¹ is 2-hydroxyethyl and R² is methyl;
(ix) R¹ is 1-hydroxy-2-propyl and R² is methyl;
(x) R¹ is 2-hydroxy-2-propyl and R² is phenyl;
(xi) R¹ is 2-hydroxy-2-propyl and R² is pyridyl;
(xii) R¹ is 2-hydroxy-2-propyl and R² is pyrazolyl;
(xiii) R¹ is 2-hydroxy-2-propyl, and R² is S(O₂)CH₃;
(xiv) R¹ is 2-hydroxy-2-propyl and R² is chloro;
(xv) R¹ is 2-hydroxy-2-propyl and R² is fluoro;
(xvi) R¹ is 1-hydroxy-1-cyclopropyl, and R² is methyl;
(xvii) R¹ is 1-hydroxy-1-cyclobutyl, and R² is methyl;
(xviii) R¹ is 1-hydroxy-1-cyclopentyl, and R² is methyl;
(xix) R¹ is 1-hydroxy-1-cyclohexyl, and R² is methyl;
(xx) R¹ is morpholinyl, and R² is methyl;
(xxi) R¹ is 1,3-dioxolan-2-yl, and R² is methyl;
(xxii) R⁸ is 1,3-dioxolan-2-yl, and R² is fluoro;
(xxiii) R¹ is 1,3-dioxolan-2-yl, and R² is chloro;
(xxiv) R¹ is COCH₃, and R² is methyl;
(xxv) R¹ is 2-methoxy-2-propyl, and R² is methyl;
(xxvi) R¹ is (dimethylamino)methyl, and R² is methyl;
(xxvii) R² is 1-hydroxy-2-methylpropan-2-yl, and R¹ is methyl;
(xxviii) R² is 2-hydroxy-2-propyl and R¹ is methyl;
(xxix) R² is 2-hydroxy-2-propyl and R¹ is isopropyl;
(xxx) R² is 2-hydroxy-2-propyl and R¹ is 1-hydroxyethyl;
(xxxi) R² is hydroxymethyl and R¹ is methyl;
(xxxii) R² is 1-hydroxyethyl and R¹ is methyl;
(xxxiii) R² is 2-hydroxyethyl and R¹ is methyl;
(xxxiv) R² is 1-hydroxy-2-propyl and R¹ is methyl;
(xxxv) R² is 2-hydroxy-2-propyl and R¹ is phenyl;
(xxxvi) R² is C₁-C₆ alkyl optionally substituted with one or more hydroxy, and R¹ is 5- to 10-membered heteroaryl;
(xxxvii) R² is 2-hydroxy-2-propyl and R¹ is pyridyl;
(xxxviii) R² is 2-hydroxy-2-propyl and R¹ is pyrazolyl;
(xxxix) R² is C₁-C₆ alkyl optionally substituted with one or more hydroxy, and R¹ is S(O₂)CH₃;
(xl) R² is 2-hydroxy-2-propyl and R¹ is chloro;
(xli) R² is 2-hydroxy-2-propyl and R¹ is fluoro;
(xlii) R² is C₃-C₇ cycloalkyl optionally substituted with one or more hydroxy, and R¹ is C₁-C₆ alkyl;
(xliii) R² is 1-hydroxy-1-cyclopropyl, and R¹ is methyl;
(xliv) R² is 1-hydroxy-1-cyclobutyl, and R¹ is methyl;
(xlv) R² is 1-hydroxy-1-cyclopentyl, and R¹ is methyl;
(xlvi) R² is 1-hydroxy-1-cyclohexyl, and R¹ is methyl;
(xlvii) R² is morpholinyl, and R¹ is methyl;
(xlviii) R² is 1,3-dioxolan-2-yl, and R¹ is methyl;
(xlix) R² is 1,3-dioxolan-2-yl, and R¹ is fluoro;
(l) R² is 1,3-dioxolan-2-yl, and R¹ is chloro;
(li) R² is C₁-C₆ alkyl optionally substituted with one or more oxo, and R¹ is methyl;
(lii) R² is COCH₃, and R¹ is methyl;
(liii) R² is 2-methoxy-2-propyl, and R¹ is methyl;
or
(liv) R² is (dimethylamino)methyl, and R¹ is methyl.

In some embodiments, of the compound of formula AA,

the substituted ring A is

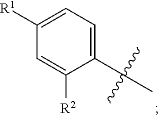

and R¹ and R² are one of the following combinations:
(i) R¹ is C₁-C₆ alkyl optionally substituted with one or more hydroxy, and R² is C₁-C₆ alkyl optionally substituted with one or more hydroxy;
(ii) R¹ is C₁-C₆ alkyl optionally substituted with one or more hydroxy, and R² is C₆-C₁₀ aryl;
(iii) R¹ is C₁-C₆ alkyl optionally substituted with one or more hydroxy, and R² is 5- to 10-membered heteroaryl;
(iv) R¹ is C₁-C₆ alkyl optionally substituted with one or more hydroxy, and R² is SF₅;
(v) R¹ is C₁-C₆ alkyl optionally substituted with one or more hydroxy, and R² is S(O₂)C₁-C₆ alkyl;
(vi) R¹ is C₁-C₆ alkyl optionally substituted with one or more hydroxy, and R² is halo;
(vii) R¹ is C₃-C₇ cycloalkyl optionally substituted with one or more hydroxy, and R² is C₁-C₆ alkyl;
(viii) R¹ is 3- to 7-membered heterocycloalkyl optionally substituted with one or more hydroxy, and R² is C₁-C₆ alkyl;
(ix) R¹ is 3- to 7-membered heterocycloalkyl optionally substituted with one or more hydroxy, and R² is halo;
(x) R¹ is C₁-C₆ alkyl optionally substituted with one or more oxo, and R² is methyl;
(xi) R¹ is C₁-C₆ alkyl optionally substituted with one or more C₁-C₆ alkoxy, and R² is C₁-C₆ alkyl;
(xii) R¹ is C₁-C₆ alkyl optionally substituted with one or more NR⁸R⁹, and R² is C₁-C₆ alkyl;
(xiii) R¹ is C₁-C₆ alkyl optionally substituted with one or more NR⁸R⁹, and R² is halo;
(xiv) R² is C₁-C₆ alkyl optionally substituted with one or more hydroxy, and R¹ is C₆-C₁₀ aryl;
(xv) R² is C₁-C₆ alkyl optionally substituted with one or more hydroxy, and R¹ is 5- to 10-membered heteroaryl;
(xvi) R² is C₁-C₆ alkyl optionally substituted with one or more hydroxy, and R¹ is SF₅.
(xvii) R² is C₁-C₆ alkyl optionally substituted with one or more hydroxy, and R¹ is S(O₂)C₁-C₆ alkyl;
(xviii) R² is C₁-C₆ alkyl optionally substituted with one or more hydroxy, and R¹ is halo;
(xix) R² is C₃-C₇ cycloalkyl optionally substituted with one or more hydroxy, and R¹ is C₁-C₆ alkyl;
(xx) R² is 3- to 7-membered heterocycloalkyl optionally substituted with one or more hydroxy, and R¹ is C₁-C₆ alkyl;
(xxi) R² is 3- to 7-membered heterocycloalkyl optionally substituted with one or more hydroxy, and R¹ is halo;
(xxii) R² is C₁-C₆ alkyl optionally substituted with one or more oxo, and R¹ is methyl;
(xxiii) R² is C₁-C₆ alkyl optionally substituted with one or more C₁-C₆ alkoxy, and R¹ is C₁-C₆ alkyl;
(xxiv) R² is C₁-C₆ alkyl optionally substituted with one or more NR⁸R⁹, and R¹ is C₁-C₆ alkyl;
or
(xxv) R² is C₁-C₆ alkyl optionally substituted with one or more NR⁸R⁹, and R¹ is halo.

In some embodiments, of the compound of formula AA,

the substituted ring A is

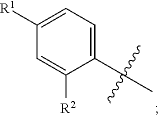

and R¹ and R² are one of the following combinations:
(i) R¹ is 1-hydroxy-2-methylpropan-2-yl, and R² is methyl;
(ii) R¹ is 2-hydroxy-2-propyl and R² is methyl;
(iii) R¹ is 2-hydroxy-2-propyl and R² is isopropyl;
(iv) R¹ is 2-hydroxy-2-propyl and R² is 2-hydroxy-2-propyl;
(v) R¹ is 2-hydroxy-2-propyl and R² is 1-hydroxyethyl;
(vi) R¹ is hydroxymethyl and R² is methyl;
(vii) R¹ is 1-hydroxyethyl and R² is methyl;
(viii) R¹ is 2-hydroxyethyl and R² is methyl;
(ix) R¹ is 1-hydroxy-2-propyl and R² is methyl;
(x) R¹ is 2-hydroxy-2-propyl and R² is phenyl;
(xi) R¹ is 2-hydroxy-2-propyl and R² is pyridyl;
(xii) R¹ is 2-hydroxy-2-propyl and R² is pyrazolyl;
(xiii) R¹ is 2-hydroxy-2-propyl, and R² is S(O₂)CH₃;
(xiv) R¹ is 2-hydroxy-2-propyl and R² is chloro;
(xv) R¹ is 2-hydroxy-2-propyl and R² is fluoro;
(xvi) R¹ is 1-hydroxy-1-cyclopropyl, and R² is methyl;
(xvii) R¹ is 1-hydroxy-1-cyclobutyl, and R² is methyl;
(xviii) R¹ is 1-hydroxy-1-cyclopentyl, and R² is methyl;
(xix) R¹ is 1-hydroxy-1-cyclohexyl, and R² is methyl;
(xx) R¹ is morpholinyl, and R² is methyl;
(xxi) R¹ is 1,3-dioxolan-2-yl, and R² is methyl;
(xxii) R¹ is 1,3-dioxolan-2-yl, and R² is fluoro;
(xxiii) R¹ is 1,3-dioxolan-2-yl, and R² is chloro;
(xxiv) R¹ is COCH₃, and R² is methyl;
(xxv) R¹ is 2-methoxy-2-propyl, and R² is methyl;
(xxvi) R¹ is (dimethylamino)methyl, and R² is methyl;
(xxvii) R² is 1-hydroxy-2-methylpropan-2-yl, and R¹ is methyl;
(xxviii) R² is 2-hydroxy-2-propyl and R¹ is methyl;
(xxix) R² is 2-hydroxy-2-propyl and R¹ is isopropyl;
(xxx) R² is 2-hydroxy-2-propyl and R¹ is 1-hydroxyethyl;
(xxxi) R² is hydroxymethyl and R¹ is methyl;
(xxxii) R² is 1-hydroxyethyl and R¹ is methyl;
(xxxiii) R² is 2-hydroxyethyl and R¹ is methyl;
(xxxiv) R² is 1-hydroxy-2-propyl and R¹ is methyl;
(xxxv) R² is 2-hydroxy-2-propyl and R¹ is phenyl;
(xxxvi) R² is C₁-C₆ alkyl optionally substituted with one or more hydroxy, and R¹ is 5- to 10-membered heteroaryl;
(xxxvii) R² is 2-hydroxy-2-propyl and R¹ is pyridyl;
(xxxviii) R² is 2-hydroxy-2-propyl and R¹ is pyrazolyl;
(xxxix) R² is C₁-C₆ alkyl optionally substituted with one or more hydroxy, and R¹ is S(O₂)CH₃;
(xl) R² is 2-hydroxy-2-propyl and R¹ is chloro;
(xli) R² is 2-hydroxy-2-propyl and R¹ is fluoro;
(xlii) R² is C₃-C₇ cycloalkyl optionally substituted with one or more hydroxy, and R¹ is C₁-C₆ alkyl;
(xliii) R² is 1-hydroxy-1-cyclopropyl, and R¹ is methyl;
(xliv) R² is 1-hydroxy-1-cyclobutyl, and R¹ is methyl;
(xlv) R² is 1-hydroxy-1-cyclopentyl, and R¹ is methyl;
(xlvi) R² is 1-hydroxy-1-cyclohexyl, and R¹ is methyl;
(xlvii) R² is morpholinyl, and R¹ is methyl;
(xlviii) R² is 1,3-dioxolan-2-yl, and R¹ is methyl;
(xlix) R² is 1,3-dioxolan-2-yl, and R¹ is fluoro;
(l) R² is 1,3-dioxolan-2-yl, and R¹ is chloro;
(li) R² is C₁-C₆ alkyl optionally substituted with one or more oxo, and R¹ is methyl;
(lii) R² is COCH₃, and R¹ is methyl;
(liii) R² is 2-methoxy-2-propyl, and R¹ is methyl;
or
(liv) R² is (dimethylamino)methyl, and R¹ is methyl.

In some embodiments, of the compound of formula AA,

the substituted ring A is

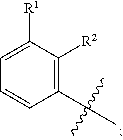

and R¹ and R² are one of the following combinations:
(i) R¹ is C₁-C₆ alkyl optionally substituted with one or more hydroxy, and R² is C₁-C₆ alkyl optionally substituted with one or more hydroxy;
(ii) R¹ is C₁-C₆ alkyl optionally substituted with one or more hydroxy, and R² is C₆-C₁₀ aryl;
(iii) R¹ is C₁-C₆ alkyl optionally substituted with one or more hydroxy, and R² is 5- to 10-membered heteroaryl;
(iv) R¹ is C₁-C₆ alkyl optionally substituted with one or more hydroxy, and R² is SF₅;
(v) R¹ is C₁-C₆ alkyl optionally substituted with one or more hydroxy, and R² is S(O₂)C₁-C₆ alkyl;
(vi) R¹ is C₁-C₆ alkyl optionally substituted with one or more hydroxy, and R² is halo;
(vii) R¹ is C₃-C₇ cycloalkyl optionally substituted with one or more hydroxy, and R² is C₁-C₆ alkyl;
(viii) R¹ is 3- to 7-membered heterocycloalkyl optionally substituted with one or more hydroxy, and R² is C₁-C₆ alkyl;
(ix) R¹ is 3- to 7-membered heterocycloalkyl optionally substituted with one or more hydroxy, and R² is halo;
(x) R¹ is C₁-C₆ alkyl optionally substituted with one or more oxo, and R² is methyl;
(xi) R¹ is C₁-C₆ alkyl optionally substituted with one or more C₁-C₆ alkoxy, and R² is C₁-C₆ alkyl;
(xii) R¹ is C₁-C₆ alkyl optionally substituted with one or more NR⁸R⁹, and R² is C₁-C₆ alkyl;
(xiii) R¹ is C₁-C₆ alkyl optionally substituted with one or more NR⁸R⁹, and R² is halo;
(xiv) R² is C₁-C₆ alkyl optionally substituted with one or more hydroxy, and R¹ is C₆-C₁₀ aryl;
(xv) R² is C₁-C₆ alkyl optionally substituted with one or more hydroxy, and R¹ is 5- to 10-membered heteroaryl;
(xvi) R² is C₁-C₆ alkyl optionally substituted with one or more hydroxy, and R¹ is SF₅.
(xvii) R² is C₁-C₆ alkyl optionally substituted with one or more hydroxy, and R¹ is S(O₂)C₁-C₆ alkyl;
(xviii) R² is C₁-C₆ alkyl optionally substituted with one or more hydroxy, and R¹ is halo;
(xix) R² is C₃-C₇ cycloalkyl optionally substituted with one or more hydroxy, and R¹ is C₁-C₆ alkyl;
(xx) R² is 3- to 7-membered heterocycloalkyl optionally substituted with one or more hydroxy, and R¹ is C₁-C₆ alkyl;
(xxi) R² is 3- to 7-membered heterocycloalkyl optionally substituted with one or more hydroxy, and R¹ is halo;
(xxii) R² is C₁-C₆ alkyl optionally substituted with one or more oxo, and R¹ is methyl;
(xxiii) R² is C₁-C₆ alkyl optionally substituted with one or more C₁-C₆ alkoxy, and R¹ is C₁-C₆ alkyl;
(xxiv) R² is C₁-C₆ alkyl optionally substituted with one or more NR⁸R⁹, and R¹ is C₁-C₆ alkyl;
or
(xxv) R² is C₁-C₆ alkyl optionally substituted with one or more NR⁸R⁹, and R¹ is halo.

In some embodiments, of the compound of formula AA,

the substituted ring A is

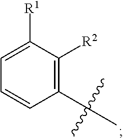

and R¹ and R² are one of the following combinations:
(i) R¹ is 1-hydroxy-2-methylpropan-2-yl, and R² is methyl;
(ii) R¹ is 2-hydroxy-2-propyl and R² is methyl;
(iii) R¹ is 2-hydroxy-2-propyl and R² is isopropyl;
(iv) R¹ is 2-hydroxy-2-propyl and R² is 2-hydroxy-2-propyl;
(v) R¹ is 2-hydroxy-2-propyl and R² is 1-hydroxyethyl;
(vi) R¹ is hydroxymethyl and R² is methyl;
(vii) R¹ is 1-hydroxyethyl and R² is methyl;
(viii) R¹ is 2-hydroxyethyl and R² is methyl;
(ix) R¹ is 1-hydroxy-2-propyl and R² is methyl;
(x) R¹ is 2-hydroxy-2-propyl and R² is phenyl;
(xi) R¹ is 2-hydroxy-2-propyl and R² is pyridyl;
(xii) R¹ is 2-hydroxy-2-propyl and R² is pyrazolyl;
(xiii) R¹ is 2-hydroxy-2-propyl, and R² is S(O₂)CH₃;
(xiv) R¹ is 2-hydroxy-2-propyl and R² is chloro;
(xv) R¹ is 2-hydroxy-2-propyl and R² is fluoro;
(xvi) R¹ is 1-hydroxy-1-cyclopropyl, and R² is methyl;
(xvii) R¹ is 1-hydroxy-1-cyclobutyl, and R² is methyl;
(xviii) R¹ is 1-hydroxy-1-cyclopentyl, and R² is methyl;
(xix) R¹ is 1-hydroxy-1-cyclohexyl, and R² is methyl;
(xx) R¹ is morpholinyl, and R² is methyl;
(xxi) R¹ is 1,3-dioxolan-2-yl, and R² is methyl;
(xxii) R¹ is 1,3-dioxolan-2-yl, and R² is fluoro;
(xxiii) R¹ is 1,3-dioxolan-2-yl, and R² is chloro;
(xxiv) R¹ is COCH₃, and R² is methyl;
(xxv) R¹ is 2-methoxy-2-propyl, and R² is methyl;
(xxvi) R¹ is (dimethylamino)methyl, and R² is methyl;
(xxvii) R² is 1-hydroxy-2-methylpropan-2-yl, and R¹ is methyl;
(xxviii) R² is 2-hydroxy-2-propyl and R¹ is methyl;
(xxix) R² is 2-hydroxy-2-propyl and R¹ is isopropyl;
(xxx) R² is 2-hydroxy-2-propyl and R¹ is 1-hydroxyethyl;
(xxxi) R² is hydroxymethyl and R¹ is methyl;
(xxxii) R² is 1-hydroxyethyl and R¹ is methyl;
(xxxiii) R² is 2-hydroxyethyl and R¹ is methyl;
(xxxiv) R² is 1-hydroxy-2-propyl and R¹ is methyl;
(xxxv) R² is 2-hydroxy-2-propyl and R¹ is phenyl;
(xxxvi) R² is C₁-C₆ alkyl optionally substituted with one or more hydroxy, and R¹ is 5- to 10-membered heteroaryl;
(xxxvii) R² is 2-hydroxy-2-propyl and R¹ is pyridyl;
(xxxviii) R² is 2-hydroxy-2-propyl and R¹ is pyrazolyl;
(xxxix) R² is C₁-C₆ alkyl optionally substituted with one or more hydroxy, and R¹ is S(O₂)CH₃;
(xl) R² is 2-hydroxy-2-propyl and R¹ is chloro;
(xli) R² is 2-hydroxy-2-propyl and R¹ is fluoro;
(xlii) R² is C₃-C₇ cycloalkyl optionally substituted with one or more hydroxy, and R¹ is C₁-C₆ alkyl;
(xliii) R² is 1-hydroxy-1-cyclopropyl, and R¹ is methyl;
(xliv) R² is 1-hydroxy-1-cyclobutyl, and R¹ is methyl;
(xlv) R² is 1-hydroxy-1-cyclopentyl, and R¹ is methyl;
(xlvi) R² is 1-hydroxy-1-cyclohexyl, and R¹ is methyl;
(xlvii) R² is morpholinyl, and R¹ is methyl;
(xlviii) R² is 1,3-dioxolan-2-yl, and R¹ is methyl;
(xlix) R² is 1,3-dioxolan-2-yl, and R¹ is fluoro;
(l) R² is 1,3-dioxolan-2-yl, and R¹ is chloro;
(li) R² is C₁-C₆ alkyl optionally substituted with one or more oxo, and R¹ is methyl;
(lii) R² is COCH₃, and R¹ is methyl;
(liii) R² is 2-methoxy-2-propyl, and R¹ is methyl;
or
(liv) R² is (dimethylamino)methyl, and R¹ is methyl.

In some embodiments, of the compound of formula AA,

the substituted ring A is

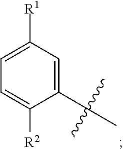

and R¹ and R² are one of the following combinations:
(i) R¹ is C₁-C₆ alkyl optionally substituted with one or more hydroxy, and R² is C₁-C₆ alkyl optionally substituted with one or more hydroxy;
(ii) R¹ is C₁-C₆ alkyl optionally substituted with one or more hydroxy, and R² is C₆-C₁₀ aryl;
(iii) R¹ is C₁-C₆ alkyl optionally substituted with one or more hydroxy, and R² is 5- to 10-membered heteroaryl;
(iv) R¹ is C₁-C₆ alkyl optionally substituted with one or more hydroxy, and R² is SF₅;
(v) R¹ is C₁-C₆ alkyl optionally substituted with one or more hydroxy, and R² is S(O₂)C₁-C₆ alkyl;
(vi) R¹ is C₁-C₆ alkyl optionally substituted with one or more hydroxy, and R² is halo;
(vii) R¹ is C₃-C₇ cycloalkyl optionally substituted with one or more hydroxy, and R² is C₁-C₆ alkyl;
(viii) R¹ is 3- to 7-membered heterocycloalkyl optionally substituted with one or more hydroxy, and R² is C₁-C₆ alkyl;
(ix) R¹ is 3- to 7-membered heterocycloalkyl optionally substituted with one or more hydroxy, and R² is halo;
(x) R¹ is C₁-C₆ alkyl optionally substituted with one or more oxo, and R² is methyl;
(xi) R¹ is C₁-C₆ alkyl optionally substituted with one or more C₁-C₆ alkoxy, and R² is C₁-C₆ alkyl;
(xii) R¹ is C₁-C₆ alkyl optionally substituted with one or more NR⁸R⁹, and R² is C₁-C₆ alkyl;
(xiii) R¹ is C₁-C₆ alkyl optionally substituted with one or more NR⁸R⁹, and R² is halo;
(xiv) R² is C₁-C₆ alkyl optionally substituted with one or more hydroxy, and R¹ is C₆-C₁₀ aryl;
(xv) R² is C₁-C₆ alkyl optionally substituted with one or more hydroxy, and R¹ is 5- to 10-membered heteroaryl;
(xvi) R² is C₁-C₆ alkyl optionally substituted with one or more hydroxy, and R¹ is SF₅.
(xvii) R² is C₁-C₆ alkyl optionally substituted with one or more hydroxy, and R¹ is S(O₂)C₁-C₆ alkyl;
(xviii) R² is C₁-C₆ alkyl optionally substituted with one or more hydroxy, and R¹ is halo;
(xix) R² is C₃-C₇ cycloalkyl optionally substituted with one or more hydroxy, and R¹ is C₁-C₆ alkyl;
(xx) R² is 3- to 7-membered heterocycloalkyl optionally substituted with one or more hydroxy, and R¹ is C₁-C₆ alkyl;
(xxi) R² is 3- to 7-membered heterocycloalkyl optionally substituted with one or more hydroxy, and R¹ is halo;
(xxii) R² is C₁-C₆ alkyl optionally substituted with one or more oxo, and R¹ is methyl;
(xxiii) R² is C₁-C₆ alkyl optionally substituted with one or more C₁-C₆ alkoxy, and R¹ is C₁-C₆ alkyl;
(xxiv) R² is C₁-C₆ alkyl optionally substituted with one or more NR⁸R⁹, and R¹ is C₁-C₆ alkyl;
or
(xxv) R² is C₁-C₆ alkyl optionally substituted with one or more NR⁸R⁹, and R¹ is halo.

In some embodiments, of the compound of formula AA,

the substituted ring A is

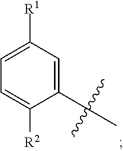

and R¹ and R² are one of the following combinations:
(i) R¹ is 1-hydroxy-2-methylpropan-2-yl, and R² is methyl;
(ii) R¹ is 2-hydroxy-2-propyl and R² is methyl;
(iii) R¹ is 2-hydroxy-2-propyl and R² is isopropyl;
(iv) R¹ is 2-hydroxy-2-propyl and R² is 2-hydroxy-2-propyl;
(v) R¹ is 2-hydroxy-2-propyl and R² is 1-hydroxyethyl;
(vi) R¹ is hydroxymethyl and R² is methyl;
(vii) R¹ is 1-hydroxyethyl and R² is methyl;
(viii) R¹ is 2-hydroxyethyl and R² is methyl;
(ix) R¹ is 1-hydroxy-2-propyl and R² is methyl;
(x) R¹ is 2-hydroxy-2-propyl and R² is phenyl;
(xi) R¹ is 2-hydroxy-2-propyl and R² is pyridyl;
(xii) R¹ is 2-hydroxy-2-propyl and R² is pyrazolyl;
(xiii) R¹ is 2-hydroxy-2-propyl, and R² is S(O₂)CH₃;
(xiv) R¹ is 2-hydroxy-2-propyl and R² is chloro;
(xv) R¹ is 2-hydroxy-2-propyl and R² is fluoro;
(xvi) R¹ is 1-hydroxy-1-cyclopropyl, and R² is methyl;
(xvii) R¹ is 1-hydroxy-1-cyclobutyl, and R² is methyl;
(xviii) R¹ is 1-hydroxy-1-cyclopentyl, and R² is methyl;
(xix) R¹ is 1-hydroxy-1-cyclohexyl, and R² is methyl;
(xx) R¹ is morpholinyl, and R² is methyl;
(xxi) R¹ is 1,3-dioxolan-2-yl, and R² is methyl;
(xxii) R¹ is 1,3-dioxolan-2-yl, and R² is fluoro;
(xxiii) R¹ is 1,3-dioxolan-2-yl, and R² is chloro;
(xxiv) R¹ is COCH₃, and R² is methyl;
(xxv) R¹ is 2-methoxy-2-propyl, and R² is methyl;
(xxvi) R¹ is (dimethylamino)methyl, and R² is methyl;
(xxvii) R² is 1-hydroxy-2-methylpropan-2-yl, and R¹ is methyl;
(xxviii) R² is 2-hydroxy-2-propyl and R¹ is methyl;
(xxix) R² is 2-hydroxy-2-propyl and R¹ is isopropyl;
(xxx) R² is 2-hydroxy-2-propyl and R¹ is 1-hydroxyethyl;
(xxxi) R² is hydroxymethyl and R¹ is methyl;
(xxxii) R² is 1-hydroxyethyl and R¹ is methyl;
(xxxiii) R² is 2-hydroxyethyl and R¹ is methyl;
(xxxiv) R² is 1-hydroxy-2-propyl and R¹ is methyl;
(xxxv) R² is 2-hydroxy-2-propyl and R¹ is phenyl;
(xxxvi) R² is C₁-C₆ alkyl optionally substituted with one or more hydroxy, and R¹ is 5- to 10-membered heteroaryl;
(xxxvii) R² is 2-hydroxy-2-propyl and R¹ is pyridyl;
(xxxviii) R² is 2-hydroxy-2-propyl and R¹ is pyrazolyl;
(xxxix) R² is C₁-C₆ alkyl optionally substituted with one or more hydroxy, and R¹ is S(O₂)CH₃;
(xl) R² is 2-hydroxy-2-propyl and R¹ is chloro;
(xli) R² is 2-hydroxy-2-propyl and R¹ is fluoro;
(xlii) R² is C₃-C₇ cycloalkyl optionally substituted with one or more hydroxy, and R¹ is C₁-C₆ alkyl;
(xliii) R² is 1-hydroxy-1-cyclopropyl, and R¹ is methyl;
(xliv) R² is 1-hydroxy-1-cyclobutyl, and R¹ is methyl;
(xlv) R² is 1-hydroxy-1-cyclopentyl, and R¹ is methyl;
(xlvi) R² is 1-hydroxy-1-cyclohexyl, and R¹ is methyl;
(xlvii) R² is morpholinyl, and R¹ is methyl;
(xlviii) R² is 1,3-dioxolan-2-yl, and R¹ is methyl;
(xlix) R² is 1,3-dioxolan-2-yl, and R¹ is fluoro;
(l) R² is 1,3-dioxolan-2-yl, and R¹ is chloro;
(li) R² is C₁-C₆ alkyl optionally substituted with one or more oxo, and R¹ is methyl;
(lii) R² is COCH₃, and R¹ is methyl;
(liii) R² is 2-methoxy-2-propyl, and R¹ is methyl;
or
(liv) R² is (dimethylamino)methyl, and R¹ is methyl.

In some embodiments, the optionally substituted ring A is

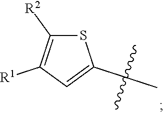

R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring that includes from 1-3 heteroatoms and/or heteroatomic groups independently selected from O, NH, NR¹³, S, S(O), and S(O)₂, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

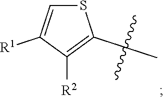

R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

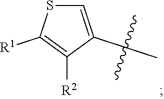

R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

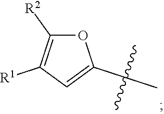

R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

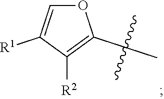

R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

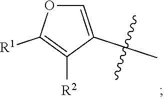

R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

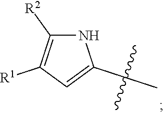

R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

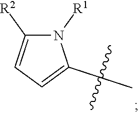

R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring that includes from 0-2 heteroatoms and/heteroatomic groups independently selected from O, NH, NR¹³, S, S(O), and S(O)₂ (in addition to the nitrogen atom attached to R¹), and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

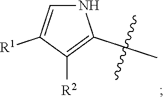

R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

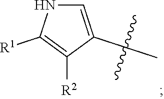

R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

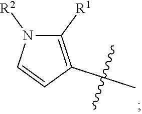

R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring that includes from 0-2 heteroatoms and/heteroatomic groups independently selected from O, NH, NR¹³, S, S(O), and S(O)₂ (in addition to the nitrogen atom attached to R²), and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

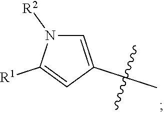

R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring that includes from 0-2 heteroatoms and/heteroatomic groups independently selected from O, NH, NR¹³, S, S(O), and S(O)₂ (in addition to the nitrogen attached to R²), and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

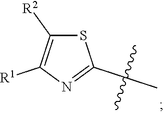

R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

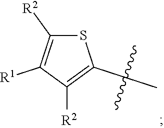

one pair of R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

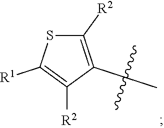

R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

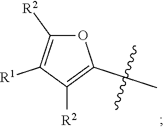

one pair of R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

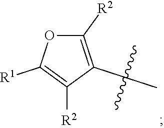

R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring that includes from 1-3 heteroatoms and/or heteroatomic groups independently selected from O, NH, NR¹³, S, S(O), and S(O)₂, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

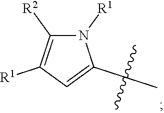

one pair of R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring wherein a) when each of the adjacent atoms is a carbon atom, then the heterocyclic ring includes from 1-3 heteroatoms and/or heteroatomic groups independently selected from O, NH, NR¹³, S, S(O), and S(O)₂; and b) when one of the adjacent atoms is a nitrogen atom, then the heterocyclic ring includes from 0-2 heteroatoms and/or heteroatomic groups independently selected from O, NH, NR¹³, S, S(O), and S(O)₂ (in addition to the aforementioned nitrogen atom attached to R¹), and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

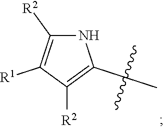

one pair of R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

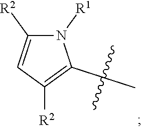

R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring that includes from 0-2 heteroatoms and/heteroatomic groups independently selected from O, NH, NR¹³, S, S(O), and S(O)₂ (in addition to the nitrogen atom attached to R¹), and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

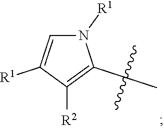

R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring that includes from 1-3 heteroatoms and/heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

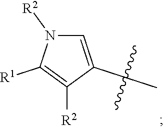

one pair of R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring wherein a) when each of the adjacent atoms is a carbon atom, then the heterocyclic ring includes from 1-3 heteroatoms and/or heteroatomic groups independently selected from O, NH, NR¹³, S, S(O), and S(O)₂; and b) when one of the adjacent atoms is a nitrogen atom, then the heterocyclic ring includes from 0-2 heteroatoms and/or heteroatomic groups independently selected from O, NH, NR¹³, S, S(O), and S(O)₂ (in addition to the aforementioned nitrogen atom attached to R²), and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

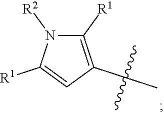

one pair of R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring that includes from 0-2 heteroatoms and/heteroatomic groups independently selected from O, NH, NR¹³, S, S(O), and S(O)₂ (in addition to the nitrogen atom(s) attached to R²), and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments of the compound of Formula AA, the optionally substituted ring A is selected from the group consisting of a 5-membered heteroaryl comprising 1-3 heteroatoms independently selected from O, N, and S, wherein the heteroatom is not bonded to the position of the heteroaryl that is bonded to the S(O)(NHR³)═N moiety;

m is 1; n is 1; and

R¹ and R² are on adjacent atoms, and taken together with the atoms connecting them, independently form one monocyclic or bicyclic C₄-C₁₂ carbocyclic ring or one monocyclic or bicyclic 5-to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments of the compound of Formula AA, the optionally substituted ring A is a pyrazolyl;

m is 1; n is 1; and

R¹ and R² are on adjacent atoms, and taken together with the atoms connecting them, independently form one monocyclic or bicyclic C₄-C₁₂ carbocyclic ring or one monocyclic or bicyclic 5-to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments of the compound of Formula AA, the optionally substituted ring A is an imidazolyl;

m is 1; n is 1; and

R¹ and R² are on adjacent atoms, and taken together with the atoms connecting them, independently form one monocyclic or bicyclic C₄-C₁₂ carbocyclic ring or one monocyclic or bicyclic 5-to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments of the compound of Formula AA, the optionally substituted ring A is a thiophenyl;

m is 1; n is 1; and

R¹ and R² are on adjacent atoms, and taken together with the atoms connecting them, independently form one monocyclic or bicyclic C₄-C₁₂ carbocyclic ring or one monocyclic or bicyclic 5-to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments of the compound of Formula AA, the optionally substituted ring A is a thiazolyl;

m is 1; n is 1; and

R¹ and R² are on adjacent atoms, and taken together with the atoms connecting them, independently form one monocyclic or bicyclic C₄-C₁₂ carbocyclic ring or one monocyclic or bicyclic 5-to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments of the compound of Formula AA, the optionally substituted ring A is

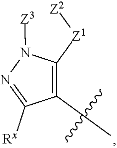

wherein R^{x} is selected from the group consisting of H and C₁-C₆ alkyl (e.g., methyl); Z¹ is selected from the group consisting of O, NH, and —CH₂— optionally substituted with 1-2 R²⁰; Z² is selected from the group consisting of NH and —CH₂— optionally substituted with 1-2 R²⁰; Z³ is selected from the group consisting of —CH₂— optionally substituted with 1-2 R²⁰, —CH₂CH₂— optionally substituted with 1-2 R²⁰, and —CH₂CH₂CH₂— optionally substituted with 1-2 R²⁰; R²⁰ is selected from the group consisting of hydroxy, halo (e.g., fluoro), oxo, C₁-C₆ alkyl (e.g., methyl or ethyl) optionally substituted with one R²¹, C₁-C₆ alkoxy (e.g., methoxy, ethoxy, or isopropoxy) optionally substituted with one R²¹, NR⁸R⁹, 3- to 10-membered heterocycloalkyl (e.g., azetidinyl or pyrrolidinyl) optionally substituted with one R²¹, or one pair of R²⁰ on the same atom, taken together with the atom connecting them, independently forms a monocyclic C₃-C₄ carbocyclic ring or a monocyclic 3- to 4-membered heterocyclic ring containing 1 O atom optionally substituted with OS(O)₂Ph; R²¹ is selected from the group consisting of halo (e.g., fluoro), NR⁸R⁹, C₂-C₆ alkynyl (e.g., ethynyl), and C₁-C₆ alkoxy (e.g., methoxy); R⁸ and R⁹ at each occurrence is independently selected from hydrogen, C₁-C₆ alkyl (e.g., methyl or ethyl), COR¹³, and CO₂R¹³; R¹³ is selected from the group consisting of: C₁-C₆ alkyl (e.g., methyl or t-butyl) and C₁-C₆ haloalkyl (e.g., trifluoromethyl).

In some embodiments of the compound of Formula AA, the optionally substituted ring A is

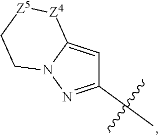

wherein Z⁴ is selected from the group consisting of —CH₂—, —C(O)—, and NH; Z⁵ is selected from the group consisting of O, NH, N—CH₃, and —CH₂—.

In some embodiments of the compound of formula AA,

the substituted ring B is

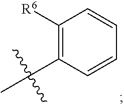

and R⁶ is selected from:
C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,

the substituted ring B is

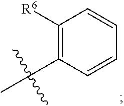

and R⁶ is selected from:
isopropyl, ethyl, methyl, trifluoromethyl, trifluoromethoxy, cyclopropyl, halo, chloro, and fluoro.

In some embodiments of the compound of formula AA,

the substituted ring B is

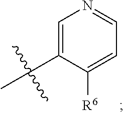

and R⁶ is selected from:
C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,

the substituted ring B is

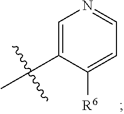

and R⁶ is selected from:
isopropyl, ethyl, methyl, trifluoromethyl, trifluoromethoxy, cyclopropyl, halo, chloro, and fluoro.

In some embodiments of the compound of formula AA,

the substituted ring B is

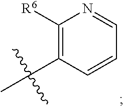

and R⁶ is selected from:
C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,

the substituted ring B is

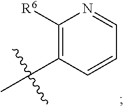

and R⁶ is selected from:
isopropyl, ethyl, methyl, trifluoromethyl, trifluoromethoxy, cyclopropyl, halo, chloro, and fluoro.

In some embodiments of the compound of formula AA,

the substituted ring B is

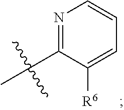

and R⁶ is selected from:
C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,

the substituted ring B is

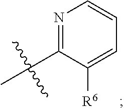

and R⁶ is selected from:
isopropyl, ethyl, methyl, trifluoromethyl, trifluoromethoxy, cyclopropyl, halo, chloro, and fluoro.

In some embodiments of the compound of formula AA,

the substituted ring B is

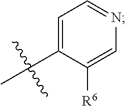

and R⁶ is selected from:
C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,

the substituted ring B is

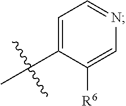

and R⁶ is selected from:
isopropyl, ethyl, methyl, trifluoromethyl, trifluoromethoxy, cyclopropyl, halo, chloro, and fluoro.

In some embodiments, of the compound of formula AA,

the substituted ring B is

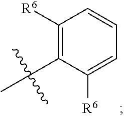

and the two R⁶ are one of the following combinations:
One R⁶ is C₁-C₆ alkyl, and the other R⁶ is C₁-C₆ alkyl optionally substituted with one or more halo;
One R⁶ is C₁-C₆ alkyl and the other R⁶ is C₁-C₆ alkyl;
One R⁶ is C₁-C₆ alkyl, and the other R⁶ is C₁-C₆ alkyl substituted with one or more halo;
One R⁶ is C₁-C₆ alkyl, and the other R⁶ is C₃-C₇ cycloalkyl;
One R⁶ is C₁-C₆ alkyl, and the other R⁶ is halo;
One R⁶ is C₁-C₆ alkyl, and the other R⁶ is cyano;
One R⁶ is C₃-C₇ cycloalkyl, and the other R⁶ is C₃-C₇ cycloalkyl;
One R⁶ is C₃-C₇ cycloalkyl, and the other R⁶ is halo;
One R⁶ is cyclopropyl and the other R⁶ is halo;
One R⁶ is C₁-C₆ alkyl, and the other R⁶ is C₁-C₆ alkoxy optionally substituted with one or more halo;
One R⁶ is C₁-C₆ alkyl, and the other R⁶ is C₁-C₆ alkoxy;
One R⁶ is C₁-C₆ alkyl, and the other R⁶ is C₁-C₆ alkoxy substituted with one or more halo;
One R⁶ is halo, and the other R⁶ is C₁-C₆ haloalkyl;
One R⁶ is halo, and the other R⁶ is C₁-C₆ haloalkoxy;
One R⁶ is C₁-C₆ alkoxy; and the other R⁶ is halo;
One R⁶ is C₁-C₆ alkoxy; and the other R⁶ is chloro.

In some embodiments, of the compound of formula AA,

the substituted ring B is

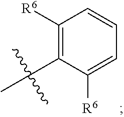

and the two R⁶ are one of the following combinations:
One R⁶ is isopropyl; and the other R⁶ is methyl;
One R⁶ is isopropyl; and the other R⁶ is n-propyl;
One R⁶ is isopropyl; and the other R⁶ is isopropyl;
One R⁶ is isopropyl; and the other R⁶ is trifluoromethyl;
One R⁶ is isopropyl; and the other R⁶ is cyclopropyl;
One R⁶ is isopropyl; and the other R⁶ is chloro;
One R⁶ is isopropyl; and the other R⁶ is fluoro;
One R⁶ is ethyl; and the other R⁶ is fluoro;
One R⁶ is isopropyl; and the other R⁶ is cyano;
One R⁶ is cyclopropyl; and the other R⁶ is cyclopropyl;
One R⁶ is cyclopropyl; and the other R⁶ is chloro;
One R⁶ is cyclopropyl; and the other R⁶ is fluoro;
One R⁶ is isopropyl; and the other R⁶ is methoxy;
One R⁶ is isopropyl; and the other R⁶ is trifluoromethoxy.

In some embodiments, of the compound of formula AA,

the substituted ring B is

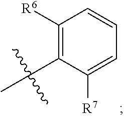

and R⁶ and R⁷ are one of the following combinations:
R⁶ is C₁-C₆ alkyl, and R⁷ is C₁-C₆ alkyl optionally substituted with one or more halo;
R⁶ is C₁-C₆ alkyl and R⁷ is C₁-C₆ alkyl;
R⁶ is C₁-C₆ alkyl, and R⁷ is C₁-C₆ alkyl substituted with one or more halo;
R⁶ is C₁-C₆ alkyl, and R⁷ is C₃-C₇ cycloalkyl;
R⁶ is C₁-C₆ alkyl, and R⁷ is halo;
R⁶ is C₁-C₆ alkyl, and R⁷ is cyano;
R⁶ is C₃-C₇ cycloalkyl, and R⁷ is C₃-C₇ cycloalkyl;
R⁶ is C₃-C₇ cycloalkyl, and R⁷ is halo;
R⁶ is cyclopropyl and R⁷ is halo;
R⁶ is C₁-C₆ alkyl, and R⁷ is C₁-C₆ alkoxy optionally substituted with one or more halo;
R⁶ is C₁-C₆ alkyl, and R⁷ is C₁-C₆ alkoxy;
R⁶ is C₁-C₆ alkyl, and R⁷ is C₁-C₆ alkoxy substituted with one or more halo;
R⁶ is halo, and R⁷ is C₁-C₆ haloalkyl;
R⁶ is halo, and R⁷ is C₁-C₆ haloalkoxy;
R⁶ is C₁-C₆ alkoxy; and R⁷ is halo;
R⁶ is C₁-C₆ alkoxy; and R⁷ is chloro;
R⁷ is C₁-C₆ alkyl, and R⁶ is C₁-C₆ alkyl optionally substituted with one or more halo;
R⁷ is C₁-C₆ alkyl, and R⁶ is C₁-C₆ alkyl substituted with one or more halo;
R⁷ is C₁-C₆ alkyl, and R⁶ is C₃-C₇ cycloalkyl;
R⁷ is C₁-C₆ alkyl, and R⁶ is halo;
R⁷ is C₁-C₆ alkyl and R⁶ is halo;
R⁷ is C₁-C₆ alkyl, and R⁶ is cyano;
R⁷ is C₃-C₇ cycloalkyl, and R⁶ is C₃-C₇ cycloalkyl;
R⁷ is C₃-C₇ cycloalkyl, and R⁶ is halo;
R⁷ is C₃-C₇ cycloalkyl and R⁶ is halo;
R⁷ is C₁-C₆ alkyl, and R⁶ is C₁-C₆ alkoxy optionally substituted with one or more halo;
R⁷ is C₁-C₆ alkyl, and R⁶ is C₁-C₆ alkoxy;
R⁷ is C₁-C₆ alkyl, and R⁶ is C₁-C₆ alkoxy substituted with one or more halo;
R⁷ is halo, and R⁶ is C₁-C₆ haloalkyl;
R⁷ is halo, and R⁶ is C₁-C₆ haloalkoxy;
R⁷ is C₁-C₆ alkoxy; and R⁶ is halo;
R⁷ is C₁-C₆ alkoxy; and R⁶ is chloro;

In some embodiments, of the compound of formula AA,

the substituted ring B is

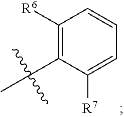

and R⁶ and R⁷ are one of the following combinations:
R⁶ is isopropyl; and R⁷ is methyl;
R⁶ is isopropyl; and R⁷ is isopropyl;
R⁶ is isopropyl; and R⁷ is trifluoromethyl;
R⁶ is isopropyl; and R⁷ is cyclopropyl;
R⁶ is isopropyl; and R⁷ is chloro;
R⁶ is isopropyl; and R⁷ is fluoro;
R⁶ is ethyl; and R⁷ is fluoro;
R⁶ is isopropyl; and R⁷ is cyano;
R⁶ is cyclopropyl; and R⁷ is cyclopropyl;
R⁶ is cyclopropyl; and R⁷ is chloro;
R⁶ is cyclopropyl; and R⁷ is fluoro;
R⁶ is isopropyl; and R⁷ is methoxy;
R⁶ is isopropyl; and R⁷ is trifluoromethoxy;
R⁶ is chloro; and R⁷ is trifluoromethyl;
R⁶ is chloro; and R⁷ is trifluoromethoxy;
R⁷ is isopropyl; and R⁶ is methyl;
R⁷ is isopropyl; and R⁶ is trifluoromethyl;
R⁷ is isopropyl; and R⁶ is cyclopropyl;
R⁷ is isopropyl; and R⁶ is chloro;
R⁷ is ethyl; and R⁶ is fluoro;
R⁷ is isopropyl; and R⁶ is cyano;
R⁷ is cyclopropyl; and R⁶ is cyclopropyl;
R⁷ is cyclopropyl; and R⁶ is chloro;
R⁷ is cyclopropyl; and R⁶ is fluoro;
R⁷ is isopropyl; and R⁶ is methoxy;
R⁷ is isopropyl; and R⁶ is trifluoromethoxy;
R⁷ is chloro; and R⁶ is trifluoromethyl;
R⁷ is chloro; and R⁶ is trifluoromethoxy.

In some embodiments, of the compound of formula AA,

the substituted ring B is

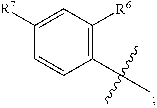

and R⁶ and R⁷ are one of the following combinations:
R⁶ is C₁-C₆ alkyl, and R⁷ is C₁-C₆ alkyl optionally substituted with one or more halo;
R⁶ is C₁-C₆ alkyl and R⁷ is C₁-C₆ alkyl;
R⁶ is C₁-C₆ alkyl, and R⁷ is C₁-C₆ alkyl substituted with one or more halo;
R⁶ is C₁-C₆ alkyl, and R⁷ is C₃-C₇ cycloalkyl;
R⁶ is C₁-C₆ alkyl, and R⁷ is halo;
R⁶ is C₁-C₆ alkyl, and R⁷ is cyano;
R⁶ is C₃-C₇ cycloalkyl, and R⁷ is C₃-C₇ cycloalkyl;
R⁶ is C₃-C₇ cycloalkyl, and R⁷ is halo;
R⁶ is cyclopropyl and R⁷ is halo;
R⁶ is C₁-C₆ alkyl, and R⁷ is C₁-C₆ alkoxy optionally substituted with one or more halo;
R⁶ is C₁-C₆ alkyl, and R⁷ is C₁-C₆ alkoxy;
R⁶ is C₁-C₆ alkyl, and R⁷ is C₁-C₆ alkoxy substituted with one or more halo;
R⁶ is halo, and R⁷ is C₁-C₆ haloalkyl;
R⁶ is halo, and R⁷ is C₁-C₆ haloalkoxy;
R⁶ is C₁-C₆ alkoxy; and R⁷ is halo;
R⁶ is C₁-C₆ alkoxy; and R⁷ is chloro;
R⁷ is C₁-C₆ alkyl, and R⁶ is C₁-C₆ alkyl optionally substituted with one or more halo;
R⁷ is C₁-C₆ alkyl, and R⁶ is C₁-C₆ alkyl substituted with one or more halo;
R⁷ is C₁-C₆ alkyl, and R⁶ is C₃-C₇ cycloalkyl;
R⁷ is C₁-C₆ alkyl, and R⁶ is halo;
R⁷ is C₁-C₆ alkyl and R⁶ is halo;
R⁷ is C₁-C₆ alkyl, and R⁶ is cyano;
R⁷ is C₃-C₇ cycloalkyl, and R⁶ is C₃-C₇ cycloalkyl;
R⁷ is C₃-C₇ cycloalkyl, and R⁶ is halo;
R⁷ is C₃-C₇ cycloalkyl and R⁶ is halo;
R⁷ is C₁-C₆ alkyl, and R⁶ is C₁-C₆ alkoxy optionally substituted with one or more halo;
R⁷ is C₁-C₆ alkyl, and R⁶ is C₁-C₆ alkoxy;
R⁷ is C₁-C₆ alkyl, and R⁶ is C₁-C₆ alkoxy substituted with one or more halo;
R⁷ is halo, and R⁶ is C₁-C₆ haloalkyl;
R⁷ is halo, and R⁶ is C₁-C₆ haloalkoxy;
R⁷ is C₁-C₆ alkoxy; and R⁶ is halo;
R⁷ is C₁-C₆ alkoxy; and R⁶ is chloro.

In some embodiments, of the compound of formula AA,

the substituted ring B is

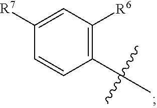

and R⁶ and R⁷ are one of the following combinations:
R⁶ is isopropyl; and R⁷ is methyl;
R⁶ is isopropyl; and R⁷ is isopropyl;
R⁶ is isopropyl; and R⁷ is trifluoromethyl;
R⁶ is isopropyl; and R⁷ is cyclopropyl;
R⁶ is isopropyl; and R⁷ is chloro;
R⁶ is isopropyl; and R⁷ is fluoro;
R⁶ is ethyl; and R⁷ is fluoro;
R⁶ is isopropyl; and R⁷ is cyano;
R⁶ is cyclopropyl; and R⁷ is cyclopropyl;
R⁶ is cyclopropyl; and R⁷ is chloro;
R⁶ is cyclopropyl; and R⁷ is fluoro;
R⁶ is isopropyl; and R⁷ is methoxy;
R⁶ is isopropyl; and R⁷ is trifluoromethoxy;
R⁶ is chloro; and R⁷ is trifluoromethyl;
R⁶ is chloro; and R⁷ is trifluoromethoxy;
R⁷ is isopropyl; and R⁶ is methyl;
R⁷ is isopropyl; and R⁶ is trifluoromethyl;
R⁷ is isopropyl; and R⁶ is cyclopropyl;
R⁷ is isopropyl; and R⁶ is chloro;
R⁷ is ethyl; and R⁶ is fluoro;
R⁷ is isopropyl; and R⁶ is cyano;
R⁷ is cyclopropyl; and R⁶ is cyclopropyl;
R⁷ is cyclopropyl; and R⁶ is chloro;
R⁷ is cyclopropyl; and R⁶ is fluoro;
R⁷ is isopropyl; and R⁶ is methoxy;
R⁷ is isopropyl; and R⁶ is trifluoromethoxy;
R⁷ is chloro; and R⁶ is trifluoromethyl;
R⁷ is chloro; and R⁶ is trifluoromethoxy.

In some embodiments, of the compound of formula AA,

the substituted ring B is

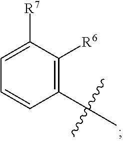

and R⁶ and R⁷ are one of the following combinations:
R⁶ is C₁-C₆ alkyl, and R⁷ is C₁-C₆ alkyl optionally substituted with one or more halo;
R⁶ is C₁-C₆ alkyl and R⁷ is C₁-C₆ alkyl;
R⁶ is C₁-C₆ alkyl, and R⁷ is C₁-C₆ alkyl substituted with one or more halo;
R⁶ is C₁-C₆ alkyl, and R⁷ is C₃-C₇ cycloalkyl;
R⁶ is C₁-C₆ alkyl, and R⁷ is halo;
R⁶ is C₁-C₆ alkyl, and R⁷ is cyano;
R⁶ is C₃-C₇ cycloalkyl, and R⁷ is C₃-C₇ cycloalkyl;
R⁶ is C₃-C₇ cycloalkyl, and R⁷ is halo;
R⁶ is cyclopropyl and R⁷ is halo;
R⁶ is C₁-C₆ alkyl, and R⁷ is C₁-C₆ alkoxy optionally substituted with one or more halo;
R⁶ is C₁-C₆ alkyl, and R⁷ is C₁-C₆ alkoxy;
R⁶ is C₁-C₆ alkyl, and R⁷ is C₁-C₆ alkoxy substituted with one or more halo;
R⁶ is halo, and R⁷ is C₁-C₆ haloalkyl;
R⁶ is halo, and R⁷ is C₁-C₆ haloalkoxy;
R⁶ is C₁-C₆ alkoxy; and R⁷ is halo;
R⁶ is C₁-C₆ alkoxy; and R⁷ is chloro;
R⁷ is C₁-C₆ alkyl, and R⁶ is C₁-C₆ alkyl optionally substituted with one or more halo;
R⁷ is C₁-C₆ alkyl, and R⁶ is C₁-C₆ alkyl substituted with one or more halo;
R⁷ is C₁-C₆ alkyl, and R⁶ is C₃-C₇ cycloalkyl;
R⁷ is C₁-C₆ alkyl, and R⁶ is halo;
R⁷ is C₁-C₆ alkyl and R⁶ is halo;
R⁷ is C₁-C₆ alkyl, and R⁶ is cyano;
R⁷ is C₃-C₇ cycloalkyl, and R⁶ is C₃-C₇ cycloalkyl;
R⁷ is C₃-C₇ cycloalkyl, and R⁶ is halo;
R⁷ is C₃-C₇ cycloalkyl and R⁶ is halo;
R⁷ is C₁-C₆ alkyl, and R⁶ is C₁-C₆ alkoxy optionally substituted with one or more halo;
R⁷ is C₁-C₆ alkyl, and R⁶ is C₁-C₆ alkoxy;
R⁷ is C₁-C₆ alkyl, and R⁶ is C₁-C₆ alkoxy substituted with one or more halo;
R⁷ is halo, and R⁶ is C₁-C₆ haloalkyl;
R⁷ is halo, and R⁶ is C₁-C₆ haloalkoxy;
R⁷ is C₁-C₆ alkoxy; and R⁶ is halo;
R⁷ is C₁-C₆ alkoxy; and R⁶ is chloro
R⁶ and R⁷ on adjacent atoms taken together with the atoms connecting them form a C₄-C₆ aliphatic carbocyclic ring optionally substituted with one or more hydroxy, oxo, or C₁-C₆ alkyl; or
R⁶ and R⁷ on adjacent atoms taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl.

In some embodiments, of the compound of formula AA,

the substituted ring B is

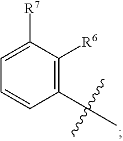

and R⁶ and R⁷ are one of the following combinations:
R⁶ is isopropyl; and R⁷ is methyl;
R⁶ is isopropyl; and R⁷ is isopropyl;
R⁶ is isopropyl; and R⁷ is trifluoromethyl;
R⁶ is isopropyl; and R⁷ is cyclopropyl;
R⁶ is isopropyl; and R⁷ is chloro;
R⁶ is isopropyl; and R⁷ is fluoro;
R⁶ is ethyl; and R⁷ is fluoro;
R⁶ is isopropyl; and R⁷ is cyano;
R⁶ is cyclopropyl; and R⁷ is cyclopropyl;
R⁶ is cyclopropyl; and R⁷ is chloro;
R⁶ is cyclopropyl; and R⁷ is fluoro;
R⁶ is isopropyl; and R⁷ is methoxy;
R⁶ is isopropyl; and R⁷ is trifluoromethoxy;
R⁶ is chloro; and R⁷ is trifluoromethyl;
R⁶ is chloro; and R⁷ is trifluoromethoxy;
R⁷ is isopropyl; and R⁶ is methyl;
R⁷ is isopropyl; and R⁶ is trifluoromethyl;
R⁷ is isopropyl; and R⁶ is cyclopropyl;
R⁷ is isopropyl; and R⁶ is chloro;
R⁷ is ethyl; and R⁶ is fluoro;
R⁷ is isopropyl; and R⁶ is cyano;
R⁷ is cyclopropyl; and R⁶ is cyclopropyl;
R⁷ is cyclopropyl; and R⁶ is chloro;
R⁷ is cyclopropyl; and R⁶ is fluoro;
R⁷ is isopropyl; and R⁶ is methoxy;
R⁷ is isopropyl; and R⁶ is trifluoromethoxy;
R⁷ is chloro; and R⁶ is trifluoromethyl;
R⁷ is chloro; and R⁶ is trifluoromethoxy;
R⁶ and R⁷ on adjacent atoms taken together with the atoms connecting them form a C₄ aliphatic carbocyclic ring;
R⁶ and R⁷ on adjacent atoms taken together with the atoms connecting them form a C₅ aliphatic carbocyclic ring;
R⁶ and R⁷ on adjacent atoms taken together with the atoms connecting them form a C₆ aliphatic carbocyclic ring;
R⁶ and R⁷ on adjacent atoms taken together with the atoms connecting them form a 5-membered heterocyclic ring containing 1 heteroatoms independently selected from O, N, and S;
R⁶ and R⁷ on adjacent atoms taken together with the atoms connecting them form a 6-membered heterocyclic ring containing 1 heteroatoms independently selected from O, N, and S; or
R⁶ and R⁷ on adjacent atoms taken together with the atoms connecting them form a C₅ aliphatic carbocyclic ring.

In some embodiments, of the compound of formula AA,

the substituted ring B is

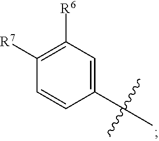

and R⁶ and R⁷ are one of the following combinations:
R⁶ is C₁-C₆ alkyl, and R⁷ is C₁-C₆ alkyl optionally substituted with one or more halo;
R⁶ is C₁-C₆ alkyl and R⁷ is C₁-C₆ alkyl;
R⁶ is C₁-C₆ alkyl, and R⁷ is C₁-C₆ alkyl substituted with one or more halo;
R⁶ is C₁-C₆ alkyl, and R⁷ is C₃-C₇ cycloalkyl;
R⁶ is C₁-C₆ alkyl, and R⁷ is halo;
R⁶ is C₁-C₆ alkyl, and R⁷ is cyano;
R⁶ is C₃-C₇ cycloalkyl, and R⁷ is C₃-C₇ cycloalkyl;
R⁶ is C₃-C₇ cycloalkyl, and R⁷ is halo;
R⁶ is cyclopropyl and R⁷ is halo;
R⁶ is C₁-C₆ alkyl, and R⁷ is C₁-C₆ alkoxy optionally substituted with one or more halo;
R⁶ is C₁-C₆ alkyl, and R⁷ is C₁-C₆ alkoxy;
R⁶ is C₁-C₆ alkyl, and R⁷ is C₁-C₆ alkoxy substituted with one or more halo;
R⁶ is halo, and R⁷ is C₁-C₆ haloalkyl;
R⁶ is halo, and R⁷ is C₁-C₆ haloalkoxy;
R⁶ is C₁-C₆ alkoxy; and R⁷ is halo;
R⁶ is C₁-C₆ alkoxy; and R⁷ is chloro;
R⁷ is C₁-C₆ alkyl, and R⁶ is C₁-C₆ alkyl optionally substituted with one or more halo;
R⁷ is C₁-C₆ alkyl, and R⁶ is C₁-C₆ alkyl substituted with one or more halo;
R⁷ is C₁-C₆ alkyl, and R⁶ is C₃-C₇ cycloalkyl;
R⁷ is C₁-C₆ alkyl, and R⁶ is halo;
R⁷ is C₁-C₆ alkyl and R⁶ is halo;
R⁷ is C₁-C₆ alkyl, and R⁶ is cyano;
R⁷ is C₃-C₇ cycloalkyl, and R⁶ is C₃-C₇ cycloalkyl;
R⁷ is C₃-C₇ cycloalkyl, and R⁶ is halo;
R⁷ is C₃-C₇ cycloalkyl and R⁶ is halo;
R⁷ is C₁-C₆ alkyl, and R⁶ is C₁-C₆ alkoxy optionally substituted with one or more halo;
R⁷ is C₁-C₆ alkyl, and R⁶ is C₁-C₆ alkoxy;
R⁷ is C₁-C₆ alkyl, and R⁶ is C₁-C₆ alkoxy substituted with one or more halo;
R⁷ is halo, and R⁶ is C₁-C₆ haloalkyl;
R⁷ is halo, and R⁶ is C₁-C₆ haloalkoxy;
R⁷ is C₁-C₆ alkoxy; and R⁶ is halo;
R⁷ is C₁-C₆ alkoxy; and R⁶ is chloro;
R⁶ and R⁷ on adjacent atoms taken together with the atoms connecting them form a C₄-C₆ aliphatic carbocyclic ring optionally substituted with one or more hydroxy, oxo, or C₁-C₆ alkyl; or
R⁶ and R⁷ on adjacent atoms taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl.

In some embodiments, of the compound of formula AA,

the substituted ring B is

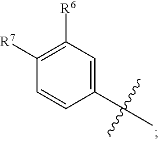

and R⁶ and R⁷ are one of the following combinations:
R⁶ is isopropyl; and R⁷ is methyl;
R⁶ is isopropyl; and R⁷ is isopropyl;
R⁶ is isopropyl; and R⁷ is trifluoromethyl;
R⁶ is isopropyl; and R⁷ is cyclopropyl;
R⁶ is isopropyl; and R⁷ is chloro;
R⁶ is isopropyl; and R⁷ is fluoro;
R⁶ is ethyl; and R⁷ is fluoro;
R⁶ is isopropyl; and R⁷ is cyano;
R⁶ is cyclopropyl; and R⁷ is cyclopropyl;
R⁶ is cyclopropyl; and R⁷ is chloro;
R⁶ is cyclopropyl; and R⁷ is fluoro;
R⁶ is isopropyl; and R⁷ is methoxy;
R⁶ is isopropyl; and R⁷ is trifluoromethoxy;
R⁶ is chloro; and R⁷ is trifluoromethyl;
R⁶ is chloro; and R⁷ is trifluoromethoxy;
R⁷ is isopropyl; and R⁶ is methyl;
R⁷ is isopropyl; and R⁶ is trifluoromethyl;
R⁷ is isopropyl; and R⁶ is cyclopropyl;
R⁷ is isopropyl; and R⁶ is chloro;
R⁷ is ethyl; and R⁶ is fluoro;
R⁷ is isopropyl; and R⁶ is cyano;
R⁷ is cyclopropyl; and R⁶ is cyclopropyl;
R⁷ is cyclopropyl; and R⁶ is chloro;
R⁷ is cyclopropyl; and R⁶ is fluoro;
R⁷ is isopropyl; and R⁶ is methoxy;
R⁷ is isopropyl; and R⁶ is trifluoromethoxy;
R⁷ is chloro; and R⁶ is trifluoromethyl;
R⁷ is chloro; and R⁶ is trifluoromethoxy;
R⁶ and R⁷ on adjacent atoms taken together with the atoms connecting them form a C₄ aliphatic carbocyclic ring;
R⁶ and R⁷ on adjacent atoms taken together with the atoms connecting them form a C₅ aliphatic carbocyclic ring;
R⁶ and R⁷ on adjacent atoms taken together with the atoms connecting them form a C₆ aliphatic carbocyclic ring;
R⁶ and R⁷ on adjacent atoms taken together with the atoms connecting them form a 5-membered heterocyclic ring containing 1 heteroatoms independently selected from O, N, and S;
R⁶ and R⁷ on adjacent atoms taken together with the atoms connecting them form a 6-membered heterocyclic ring containing 1 heteroatoms independently selected from O, N, and S; or
R⁶ and R⁷ on adjacent atoms taken together with the atoms connecting them form a C₅ aliphatic carbocyclic ring.

In some embodiments, of the compound of formula AA,

the substituted ring B is

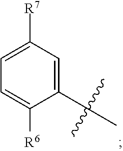

and R⁶ and R⁷ are one of the following combinations:
R⁶ is C₁-C₆ alkyl, and R⁷ is C₁-C₆ alkyl optionally substituted with one or more halo;
R⁶ is C₁-C₆ alkyl and R⁷ is C₁-C₆ alkyl;
R⁶ is C₁-C₆ alkyl, and R⁷ is C₁-C₆ alkyl substituted with one or more halo;
R⁶ is C₁-C₆ alkyl, and R⁷ is C₃-C₇ cycloalkyl;
R⁶ is C₁-C₆ alkyl, and R⁷ is halo;
R⁶ is C₁-C₆ alkyl, and R⁷ is cyano;
R⁶ is C₃-C₇ cycloalkyl, and R⁷ is C₃-C₇ cycloalkyl;
R⁶ is C₃-C₇ cycloalkyl, and R⁷ is halo;
R⁶ is cyclopropyl and R⁷ is halo;
R⁶ is C₁-C₆ alkyl, and R⁷ is C₁-C₆ alkoxy optionally substituted with one or more halo;
R⁶ is C₁-C₆ alkyl, and R⁷ is C₁-C₆ alkoxy;
R⁶ is C₁-C₆ alkyl, and R⁷ is C₁-C₆ alkoxy substituted with one or more halo;
R⁶ is halo, and R⁷ is C₁-C₆ haloalkyl;
R⁶ is halo, and R⁷ is C₁-C₆ haloalkoxy;
R⁶ is C₁-C₆ alkoxy; and R⁷ is halo;
R⁶ is C₁-C₆ alkoxy; and R⁷ is chloro;
R⁷ is C₁-C₆ alkyl, and R⁶ is C₁-C₆ alkyl optionally substituted with one or more halo;
R⁷ is C₁-C₆ alkyl, and R⁶ is C₁-C₆ alkyl substituted with one or more halo;
R⁷ is C₁-C₆ alkyl, and R⁶ is C₃-C₇ cycloalkyl;
R⁷ is C₁-C₆ alkyl, and R⁶ is halo;
R⁷ is C₁-C₆ alkyl and R⁶ is halo;
R⁷ is C₁-C₆ alkyl, and R⁶ is cyano;
R⁷ is C₃-C₇ cycloalkyl, and R⁶ is C₃-C₇ cycloalkyl;
R⁷ is C₃-C₇ cycloalkyl, and R⁶ is halo;
R⁷ is C₃-C₇ cycloalkyl and R⁶ is halo;
R⁷ is C₁-C₆ alkyl, and R⁶ is C₁-C₆ alkoxy optionally substituted with one or more halo;
R⁷ is C₁-C₆ alkyl, and R⁶ is C₁-C₆ alkoxy;
R⁷ is C₁-C₆ alkyl, and R⁶ is C₁-C₆ alkoxy substituted with one or more halo;
R⁷ is halo, and R⁶ is C₁-C₆ haloalkyl;
R⁷ is halo, and R⁶ is C₁-C₆ haloalkoxy;
R⁷ is C₁-C₆ alkoxy; and R⁶ is halo;
R⁷ is C₁-C₆ alkoxy; and R⁶ is chloro.

In some embodiments, of the compound of formula AA,

the substituted ring B is

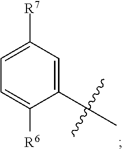

and R⁶ and R⁷ are one of the following combinations:
R⁶ is isopropyl; and R⁷ is methyl;
R⁶ is isopropyl; and R⁷ is isopropyl;
R⁶ is isopropyl; and R⁷ is trifluoromethyl;
R⁶ is isopropyl; and R⁷ is cyclopropyl;
R⁶ is isopropyl; and R⁷ is chloro;
R⁶ is isopropyl; and R⁷ is fluoro;
R⁶ is ethyl; and R⁷ is fluoro;
R⁶ is isopropyl; and R⁷ is cyano;
R⁶ is cyclopropyl; and R⁷ is cyclopropyl;
R⁶ is cyclopropyl; and R⁷ is chloro;
R⁶ is cyclopropyl; and R⁷ is fluoro;
R⁶ is isopropyl; and R⁷ is methoxy;
R⁶ is isopropyl; and R⁷ is trifluoromethoxy;
R⁶ is chloro; and R⁷ is trifluoromethyl;
R⁶ is chloro; and R⁷ is trifluoromethoxy;
R⁷ is isopropyl; and R⁶ is methyl;
R⁷ is isopropyl; and R⁶ is trifluoromethyl;
R⁷ is isopropyl; and R⁶ is cyclopropyl;
R⁷ is isopropyl; and R⁶ is chloro;
R⁷ is ethyl; and R⁶ is fluoro;
R⁷ is isopropyl; and R⁶ is cyano;
R⁷ is cyclopropyl; and R⁶ is cyclopropyl;
R⁷ is cyclopropyl; and R⁶ is chloro;
R⁷ is cyclopropyl; and R⁶ is fluoro;
R⁷ is isopropyl; and R⁶ is methoxy;
R⁷ is isopropyl; and R⁶ is trifluoromethoxy;
R⁷ is chloro; and R⁶ is trifluoromethyl;
R⁷ is chloro; and R⁶ is trifluoromethoxy.

In some embodiments, of the compound of formula AA,

the substituted ring B is

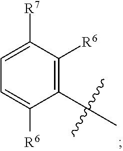

and R⁶ and R⁷ are one of the following combinations:
each R⁶ is independently C₁-C₆ alkyl, and R⁷ is C₁-C₆ alkyl optionally substituted with one or more halo;
each R⁶ is independently C₁-C₆ alkyl and R⁷ is C₁-C₆ alkyl;
each R⁶ is independently C₁-C₆ alkyl, and R⁷ is C₁-C₆ alkyl substituted with one or more halo;
each R⁶ is independently C₁-C₆ alkyl, and R⁷ is C₃-C₇ cycloalkyl;
each R⁶ is independently C₁-C₆ alkyl, and R⁷ is halo;
each R⁶ is independently C₁-C₆ alkyl, and R⁷ is cyano;
each R⁶ is independently C₃-C₇ cycloalkyl, and R⁷ is C₃-C₇ cycloalkyl;
each R⁶ is independently C₃-C₇ cycloalkyl, and R⁷ is halo;
each R⁶ is independently cyclopropyl and R⁷ is halo;
each R⁶ is independently C₁-C₆ alkyl, and R⁷ is C₁-C₆ alkoxy optionally substituted with one or more halo;
each R⁶ is independently C₁-C₆ alkyl, and R⁷ is C₁-C₆ alkoxy;
each R⁶ is independently C₁-C₆ alkyl, and R⁷ is C₁-C₆ alkoxy substituted with one or more halo;
each R⁶ is independently halo, and R⁷ is C₁-C₆ haloalkyl;
each R⁶ is independently halo, and R⁷ is C₁-C₆ haloalkoxy;
each R⁶ is independently C₁-C₆ alkoxy; and R⁷ is halo;
each R⁶ is independently C₁-C₆ alkoxy; and R⁷ is chloro;
R⁷ is C₁-C₆ alkyl, and each R⁶ is independently C₁-C₆ alkyl optionally substituted with one or more halo;
R⁷ is C₁-C₆ alkyl, and each R⁶ is independently C₁-C₆ alkyl substituted with one or more halo;
R⁷ is C₁-C₆ alkyl, and each R⁶ is independently C₃-C₇ cycloalkyl;
R⁷ is C₁-C₆ alkyl, and each R⁶ is independently halo;
R⁷ is C₁-C₆ alkyl and each R⁶ is independently halo;
R⁷ is C₁-C₆ alkyl, and each R⁶ is cyano;
R⁷ is C₃-C₇ cycloalkyl, and each R⁶ is independently C₃-C₇ cycloalkyl;
R⁷ is C₃-C₇ cycloalkyl, and each R⁶ is independently halo;
R⁷ is C₃-C₇ cycloalkyl and each R⁶ is independently halo;
R⁷ is C₁-C₆ alkyl, and each R⁶ is independently C₁-C₆ alkoxy optionally substituted with one or more halo;
R⁷ is C₁-C₆ alkyl, and each R⁶ is independently C₁-C₆ alkoxy;
R⁷ is C₁-C₆ alkyl, and each R⁶ is independently C₁-C₆ alkoxy substituted with one or more halo;
R⁷ is halo, and each R⁶ is independently C₁-C₆ haloalkyl;
R⁷ is halo, and each R⁶ is independently C₁-C₆ haloalkoxy;
R⁷ is C₁-C₆ alkoxy; and each R⁶ is independently halo;
R⁷ is C₁-C₆ alkoxy; and each R⁶ is chloro;
R⁶ and R⁷ on adjacent atoms taken together with the atoms connecting them form a C₄-C₆ aliphatic carbocyclic ring optionally substituted with one or more hydroxy, oxo, or C₁-C₆ alkyl; and one R⁶ is halo or cyano; or
R⁶ and R⁷ on adjacent atoms taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl; and one R⁶ is halo or cyano.

In some embodiments, of the compound of formula AA,

the substituted ring B is

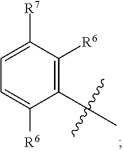

and R⁶ and R⁷ are one of the following combinations:
each R⁶ is isopropyl; and R⁷ is methyl;
each R⁶ is isopropyl; and R⁷ is isopropyl;
each R⁶ is isopropyl; and R⁷ is trifluoromethyl;
each R⁶ is isopropyl; and R⁷ is cyclopropyl;
each R⁶ is isopropyl; and R⁷ is chloro;
each R⁶ is isopropyl; and R⁷ is fluoro;
each R⁶ is ethyl; and R⁷ is fluoro;
each R⁶ is isopropyl; and R⁷ is cyano;
each R⁶ is cyclopropyl; and R⁷ is cyclopropyl;
each R⁶ is cyclopropyl; and R⁷ is chloro;
each R⁶ is cyclopropyl; and R⁷ is fluoro;
each R⁶ is isopropyl; and R⁷ is methoxy;
each R⁶ is isopropyl; and R⁷ is trifluoromethoxy;
each R⁶ is chloro; and R⁷ is trifluoromethyl;
each R⁶ is chloro; and R⁷ is trifluoromethoxy;
R⁷ is isopropyl; and each R⁶ is methyl;
R⁷ is isopropyl; and each R⁶ is trifluoromethyl;
R⁷ is isopropyl; and each R⁶ is cyclopropyl;
R⁷ is isopropyl; and each R⁶ is chloro;
R⁷ is ethyl; and each R⁶ is fluoro;
R⁷ is isopropyl; and each R⁶ is cyano;
R⁷ is cyclopropyl; and each R⁶ is cyclopropyl;
R⁷ is cyclopropyl; and each R⁶ is chloro;
R⁷ is cyclopropyl; and each R⁶ is fluoro;
R⁷ is isopropyl; and each R⁶ is methoxy;
R⁷ is isopropyl; and each R⁶ is trifluoromethoxy;
R⁷ is chloro; and each R⁶ is trifluoromethyl;
R⁷ is chloro; and each R⁶ is trifluoromethoxy;
one R⁶ is isopropyl; the other R⁶ is trifluoromethyl; and R⁷ is chloro;
R⁶ and R⁷ on adjacent atoms taken together with the atoms connecting them form a C₄ aliphatic carbocyclic ring; and one R⁶ is chloro, fluoro, or cyano;
R⁶ and R⁷ on adjacent atoms taken together with the atoms connecting them form a C₅ aliphatic carbocyclic ring; and one R⁶ is chloro, fluoro, or cyano;
R⁶ and R⁷ on adjacent atoms taken together with the atoms connecting them form a C₆ aliphatic carbocyclic ring; and one R⁶ is chloro, fluoro, or cyano;
R⁶ and R⁷ on adjacent atoms taken together with the atoms connecting them form a 5-membered heterocyclic ring containing 1 heteroatoms independently selected from O, N, and S; and one R⁶ is chloro, fluoro, or cyano;
R⁶ and R⁷ on adjacent atoms taken together with the atoms connecting them form a 6-membered heterocyclic ring containing 1 heteroatoms independently selected from O, N, and S; and one R⁶ is chloro, fluoro, or cyano; or
R⁶ and R⁷ on adjacent atoms taken together with the atoms connecting them form a C₅ aliphatic carbocyclic ring; and one R⁶ is chloro, fluoro, or cyano.

In some embodiments, of the compound of formula AA,

the substituted ring B is

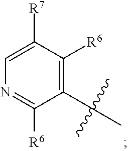

and R⁶ and R⁷ are one of the following combinations:
each R⁶ is independently C₁-C₆ alkyl, and R⁷ is C₁-C₆ alkyl optionally substituted with one or more halo;
each R⁶ is independently C₁-C₆ alkyl and R⁷ is C₁-C₆ alkyl;
each R⁶ is independently C₁-C₆ alkyl, and R⁷ is C₁-C₆ alkyl substituted with one or more halo;
each R⁶ is independently C₁-C₆ alkyl, and R⁷ is C₃-C₇ cycloalkyl;
each R⁶ is independently C₁-C₆ alkyl, and R⁷ is halo;
each R⁶ is independently C₁-C₆ alkyl, and R⁷ is cyano;
each R⁶ is independently C₃-C₇ cycloalkyl, and R⁷ is C₃-C₇ cycloalkyl;
each R⁶ is independently C₃-C₇ cycloalkyl, and R⁷ is halo;
each R⁶ is independently cyclopropyl and R⁷ is halo;
each R⁶ is independently C₁-C₆ alkyl, and R⁷ is C₁-C₆ alkoxy optionally substituted with one or more halo;
each R⁶ is independently C₁-C₆ alkyl, and R⁷ is C₁-C₆ alkoxy;
each R⁶ is independently C₁-C₆ alkyl, and R⁷ is C₁-C₆ alkoxy substituted with one or more halo;
each R⁶ is independently halo, and R⁷ is C₁-C₆ haloalkyl;
each R⁶ is independently halo, and R⁷ is C₁-C₆ haloalkoxy;
each R⁶ is independently C₁-C₆ alkoxy; and R⁷ is halo;
each R⁶ is independently C₁-C₆ alkoxy; and R⁷ is chloro;
R⁷ is C₁-C₆ alkyl, and each R⁶ is independently C₁-C₆ alkyl optionally substituted with one or more halo;
R⁷ is C₁-C₆ alkyl, and each R⁶ is independently C₁-C₆ alkyl substituted with one or more halo;
R⁷ is C₁-C₆ alkyl, and each R⁶ is independently C₃-C₇ cycloalkyl;
R⁷ is C₁-C₆ alkyl, and each R⁶ is independently halo;
R⁷ is C₁-C₆ alkyl and each R⁶ is independently halo;
R⁷ is C₁-C₆ alkyl, and each R⁶ is cyano;
R⁷ is C₃-C₇ cycloalkyl, and each R⁶ is independently C₃-C₇ cycloalkyl;
R⁷ is C₃-C₇ cycloalkyl, and each R⁶ is independently halo;
R⁷ is C₃-C₇ cycloalkyl and each R⁶ is independently halo;
R⁷ is C₁-C₆ alkyl, and each R⁶ is independently C₁-C₆ alkoxy optionally substituted with one or more halo;
R⁷ is C₁-C₆ alkyl, and each R⁶ is independently C₁-C₆ alkoxy;
R⁷ is C₁-C₆ alkyl, and each R⁶ is independently C₁-C₆ alkoxy substituted with one or more halo;
R⁷ is halo, and each R⁶ is independently C₁-C₆ haloalkyl;
R⁷ is halo, and each R⁶ is independently C₁-C₆ haloalkoxy;
R⁷ is C₁-C₆ alkoxy; and each R⁶ is independently halo;
R⁷ is C₁-C₆ alkoxy; and each R⁶ is chloro;
R⁶ and R⁷ on adjacent atoms taken together with the atoms connecting them form a C₄-C₆ aliphatic carbocyclic ring optionally substituted with one or more hydroxy, oxo, or C₁-C₆ alkyl; and one R⁶ is halo or cyano; or
R⁶ and R⁷ on adjacent atoms taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl; and one R⁶ is halo or cyano.

In some embodiments, of the compound of formula AA,

the substituted ring B is

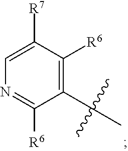

and R⁶ and R⁷ are one of the following combinations:
each R⁶ is isopropyl; and R⁷ is methyl;
each R⁶ is isopropyl; and R⁷ is isopropyl;
each R⁶ is isopropyl; and R⁷ is trifluoromethyl;
each R⁶ is isopropyl; and R⁷ is cyclopropyl;
each R⁶ is isopropyl; and R⁷ is chloro;
each R⁶ is isopropyl; and R⁷ is fluoro;
each R⁶ is ethyl; and R⁷ is fluoro;
each R⁶ is isopropyl; and R⁷ is cyano;
each R⁶ is cyclopropyl; and R⁷ is cyclopropyl;
each R⁶ is cyclopropyl; and R⁷ is chloro;
each R⁶ is cyclopropyl; and R⁷ is fluoro;
each R⁶ is isopropyl; and R⁷ is methoxy;
each R⁶ is isopropyl; and R⁷ is trifluoromethoxy;
each R⁶ is chloro; and R⁷ is trifluoromethyl;
each R⁶ is chloro; and R⁷ is trifluoromethoxy;
R⁷ is isopropyl; and each R⁶ is methyl;
R⁷ is isopropyl; and each R⁶ is trifluoromethyl;
R⁷ is isopropyl; and each R⁶ is cyclopropyl;
R⁷ is isopropyl; and each R⁶ is chloro;
R⁷ is ethyl; and each R⁶ is fluoro;
R⁷ is isopropyl; and each R⁶ is cyano;
R⁷ is cyclopropyl; and each R⁶ is cyclopropyl;
R⁷ is cyclopropyl; and each R⁶ is chloro;
R⁷ is cyclopropyl; and each R⁶ is fluoro;
R⁷ is isopropyl; and each R⁶ is methoxy;
R⁷ is isopropyl; and each R⁶ is trifluoromethoxy;
R⁷ is chloro; and each R⁶ is trifluoromethyl;
R⁷ is chloro; and each R⁶ is trifluoromethoxy;
one R⁶ is isopropyl; the other R⁶ is trifluoromethyl; and R⁷ is chloro;
R⁶ and R⁷ on adjacent atoms taken together with the atoms connecting them form a C₄ aliphatic carbocyclic ring; and one R⁶ is chloro, fluoro, or cyano;
R⁶ and R⁷ on adjacent atoms taken together with the atoms connecting them form a C₅ aliphatic carbocyclic ring; and one R⁶ is chloro, fluoro, or cyano;
R⁶ and R⁷ on adjacent atoms taken together with the atoms connecting them form a C₆ aliphatic carbocyclic ring; and one R⁶ is chloro, fluoro, or cyano;
R⁶ and R⁷ on adjacent atoms taken together with the atoms connecting them form a 5-membered heterocyclic ring containing 1 heteroatoms independently selected from O, N, and S; and one R⁶ is chloro, fluoro, or cyano;
R⁶ and R⁷ on adjacent atoms taken together with the atoms connecting them form a 6-membered heterocyclic ring containing 1 heteroatoms independently selected from O, N, and S; and one R⁶ is chloro, fluoro, or cyano; or
R⁶ and R⁷ on adjacent atoms taken together with the atoms connecting them form a C₅ aliphatic carbocyclic ring; and one R⁶ is chloro, fluoro, or cyano.

In some embodiments, of the compound of formula AA,

the substituted ring B is

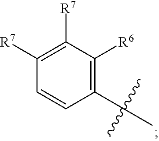

and R⁶ and R⁷ are one of the following combinations:
R⁶ is C₁-C₆ alkyl, and each R⁷ is independently C₁-C₆ alkyl optionally substituted with one or more halo;
R⁶ is C₁-C₆ alkyl and each R⁷ is independently C₁-C₆ alkyl;
R⁶ is C₁-C₆ alkyl, and each R⁷ is independently C₁-C₆ alkyl substituted with one or more halo;
R⁶ is C₁-C₆ alkyl, and each R⁷ is independently C₃-C₇ cycloalkyl;
R⁶ is C₁-C₆ alkyl, and each R⁷ is independently halo;
R⁶ is C₁-C₆ alkyl, and each R⁷ is cyano;
R⁶ is C₃-C₇ cycloalkyl, and each R⁷ is independently C₃-C₇ cycloalkyl;
R⁶ is C₃-C₇ cycloalkyl, and each R⁷ is independently halo;
R⁶ is cyclopropyl and each R⁷ is independently halo;
R⁶ is C₁-C₆ alkyl, and each R⁷ is independently C₁-C₆ alkoxy optionally substituted with one or more halo;
R⁶ is C₁-C₆ alkyl, and each R⁷ is independently C₁-C₆ alkoxy;
R⁶ is C₁-C₆ alkyl, and each R⁷ is independently C₁-C₆ alkoxy substituted with one or more halo;
R⁶ is halo, and each R⁷ is independently C₁-C₆ haloalkyl;
R⁶ is halo, and each R⁷ is independently C₁-C₆ haloalkoxy;
R⁶ is C₁-C₆ alkoxy; and each R⁷ is independently halo;
R⁶ is C₁-C₆ alkoxy; and each R⁷ is chloro;
each R⁷ is independently C₁-C₆ alkyl, and R⁶ is C₁-C₆ alkyl optionally substituted with one or more halo;
each R⁷ is independently C₁-C₆ alkyl, and R⁶ is C₁-C₆ alkyl substituted with one or more halo;
each R⁷ is independently C₁-C₆ alkyl, and R⁶ is C₃-C₇ cycloalkyl;
each R⁷ is independently C₁-C₆ alkyl, and R⁶ is halo;
each R⁷ is independently C₁-C₆ alkyl and R⁶ is halo;
each R⁷ is independently C₁-C₆ alkyl, and R⁶ is cyano;
each R⁷ is independently C₃-C₇ cycloalkyl, and R⁶ is C₃-C₇ cycloalkyl;
each R⁷ is independently C₃-C₇ cycloalkyl, and R⁶ is halo;
each R⁷ is independently C₃-C₇ cycloalkyl and R⁶ is halo;
each R⁷ is independently C₁-C₆ alkyl, and R⁶ is C₁-C₆ alkoxy optionally substituted with one or more halo;
each R⁷ is independently C₁-C₆ alkyl, and R⁶ is C₁-C₆ alkoxy;
each R⁷ is independently C₁-C₆ alkyl, and R⁶ is C₁-C₆ alkoxy substituted with one or more halo;
each R⁷ is independently halo, and R⁶ is C₁-C₆ haloalkyl;
each R⁷ is independently halo, and R⁶ is C₁-C₆ haloalkoxy;
each R⁷ is independently C₁-C₆ alkoxy; and R⁶ is halo;
each R⁷ is independently C₁-C₆ alkoxy; and R⁶ is chloro.

In some embodiments, of the compound of formula AA,

the substituted ring B is

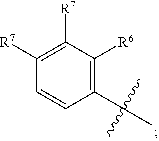

and R⁶ and R⁷ are one of the following combinations:
each R⁷ is isopropyl; and R⁶ is methyl;
each R⁷ is isopropyl; and R⁶ is isopropyl;
each R⁷ is isopropyl; and R⁶ is trifluoromethyl;
each R⁷ is isopropyl; and R⁶ is cyclopropyl;
each R⁷ is isopropyl; and R⁶ is chloro;
each R⁷ is isopropyl; and R⁶ is fluoro;
each R⁷ is ethyl; and R⁶ is fluoro;
each R⁷ is isopropyl; and R⁶ is cyano;
each R⁷ is cyclopropyl; and R⁶ is cyclopropyl;
each R⁷ is cyclopropyl; and R⁶ is chloro;
each R⁷ is cyclopropyl; and R⁶ is fluoro;
each R⁷ is isopropyl; and R⁶ is methoxy;
each R⁷ is isopropyl; and R⁶ is trifluoromethoxy;
each R⁷ is chloro; and R⁶ is trifluoromethyl;
each R⁷ is chloro; and R⁶ is trifluoromethoxy;
R⁷ is isopropyl; and each R⁶ is methyl;
R⁷ is isopropyl; and each R⁶ is trifluoromethyl;
R⁷ is isopropyl; and each R⁶ is cyclopropyl;
R⁷ is isopropyl; and each R⁶ is chloro;
R⁷ is ethyl; and each R⁶ is fluoro;
R⁷ is isopropyl; and each R⁶ is cyano;
R⁷ is cyclopropyl; and each R⁶ is cyclopropyl;
R⁷ is cyclopropyl; and each R⁶ is chloro;
R⁷ is cyclopropyl; and each R⁶ is fluoro;
R⁷ is isopropyl; and each R⁶ is methoxy;
R⁷ is isopropyl; and each R⁶ is trifluoromethoxy;
R⁷ is chloro; and each R⁶ is trifluoromethyl;
R⁷ is chloro; and each R⁶ is trifluoromethoxy.

In some embodiments, of the compound of formula AA,

the substituted ring B is

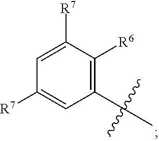

and R⁶ and R⁷ are one of the following combinations:
R⁶ is C₁-C₆ alkyl, and each R⁷ is independently C₁-C₆ alkyl optionally substituted with one or more halo;
R⁶ is C₁-C₆ alkyl and each R⁷ is independently C₁-C₆ alkyl;
R⁶ is C₁-C₆ alkyl, and each R⁷ is independently C₁-C₆ alkyl substituted with one or more halo;
R⁶ is C₁-C₆ alkyl, and each R⁷ is independently C₃-C₇ cycloalkyl;
R⁶ is C₁-C₆ alkyl, and each R⁷ is independently halo;
R⁶ is C₁-C₆ alkyl, and each R⁷ is cyano;
R⁶ is C₃-C₇ cycloalkyl, and each R⁷ is independently C₃-C₇ cycloalkyl;
R⁶ is C₃-C₇ cycloalkyl, and each R⁷ is independently halo;
R⁶ is cyclopropyl and each R⁷ is independently halo;
R⁶ is C₁-C₆ alkyl, and each R⁷ is independently C₁-C₆ alkoxy optionally substituted with one or more halo;
R⁶ is C₁-C₆ alkyl, and each R⁷ is independently C₁-C₆ alkoxy;
R⁶ is C₁-C₆ alkyl, and each R⁷ is independently C₁-C₆ alkoxy substituted with one or more halo;
R⁶ is halo, and each R⁷ is independently C₁-C₆ haloalkyl;
R⁶ is halo, and each R⁷ is independently C₁-C₆ haloalkoxy;
R⁶ is C₁-C₆ alkoxy; and each R⁷ is independently halo;
R⁶ is C₁-C₆ alkoxy; and each R⁷ is chloro;
each R⁷ is independently C₁-C₆ alkyl, and R⁶ is C₁-C₆ alkyl optionally substituted with one or more halo;
each R⁷ is independently C₁-C₆ alkyl, and R⁶ is C₁-C₆ alkyl substituted with one or more halo;
each R⁷ is independently C₁-C₆ alkyl, and R⁶ is C₃-C₇ cycloalkyl;
each R⁷ is independently C₁-C₆ alkyl, and R⁶ is halo;
each R⁷ is independently C₁-C₆ alkyl and R⁶ is halo;
each R⁷ is independently C₁-C₆ alkyl, and R⁶ is cyano;
each R⁷ is independently C₃-C₇ cycloalkyl, and R⁶ is C₃-C₇ cycloalkyl;
each R⁷ is independently C₃-C₇ cycloalkyl, and R⁶ is halo;
each R⁷ is independently C₃-C₇ cycloalkyl and R⁶ is halo;
each R⁷ is independently C₁-C₆ alkyl, and R⁶ is C₁-C₆ alkoxy optionally substituted with one or more halo;
each R⁷ is independently C₁-C₆ alkyl, and R⁶ is C₁-C₆ alkoxy;
each R⁷ is independently C₁-C₆ alkyl, and R⁶ is C₁-C₆ alkoxy substituted with one or more halo;
each R⁷ is independently halo, and R⁶ is C₁-C₆ haloalkyl;
each R⁷ is independently halo, and R⁶ is C₁-C₆ haloalkoxy;
each R⁷ is independently C₁-C₆ alkoxy; and R⁶ is halo;
each R⁶ is independently C₁-C₆ alkoxy; and R⁶ is chloro.

In some embodiments, of the compound of formula AA,

the substituted ring B is

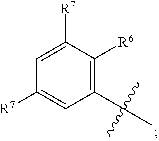

and R⁶ and R⁷ are one of the following combinations:
R⁶ is isopropyl; and each R⁷ is methyl;
R⁶ is isopropyl; and each R⁷ is isopropyl;
R⁶ is isopropyl; and each R⁷ is trifluoromethyl;
R⁶ is isopropyl; and each R⁷ is cyclopropyl;
R⁶ is isopropyl; and each R⁷ is chloro;
R⁶ is isopropyl; and each R⁷ is fluoro;
R⁶ is ethyl; and each R⁷ is fluoro;
R⁶ is isopropyl; and each R⁷ is cyano;
R⁶ is cyclopropyl; and each R⁷ is cyclopropyl;
R⁶ is cyclopropyl; and each R⁷ is chloro;
R⁶ is cyclopropyl; and each R⁷ is fluoro;
R⁶ is isopropyl; and each R⁷ is methoxy;
R⁶ is isopropyl; and R⁷ is trifluoromethoxy;
R⁶ is chloro; and each R⁷ is trifluoromethyl;
R⁶ is chloro; and each R⁷ is trifluoromethoxy;
each R⁷ is isopropyl; and R⁶ is methyl;
each R⁷ is isopropyl; and R⁶ is trifluoromethyl;
each R⁷ is isopropyl; and R⁶ is cyclopropyl;
each R⁷ is isopropyl; and R⁶ is chloro;
each R⁷ is ethyl; and R⁶ is fluoro;
each R⁷ is isopropyl; and R⁶ is cyano;
each R⁷ is cyclopropyl; and R⁶ is cyclopropyl;
each R⁷ is cyclopropyl; and R⁶ is chloro;
each R⁷ is cyclopropyl; and R⁶ is fluoro;
each R⁷ is isopropyl; and R⁶ is methoxy;
each R⁷ is isopropyl; and R⁶ is trifluoromethoxy;
each R⁷ is chloro; and R⁶ is trifluoromethyl;
each R⁷ is chloro; and R⁶ is trifluoromethoxy.

In some embodiments, of the compound of formula AA,

the substituted ring B is

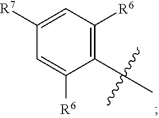

and R⁶ and R⁷ are one of the following combinations:
each R⁶ is independently C₁-C₆ alkyl, and R⁷ is C₁-C₆ alkyl optionally substituted with one or more halo;
each R⁶ is independently C₁-C₆ alkyl and R⁷ is C₁-C₆ alkyl;
each R⁶ is independently C₁-C₆ alkyl, and R⁷ is C₁-C₆ alkyl substituted with one or more halo;
each R⁶ is independently C₁-C₆ alkyl, and R⁷ is C₃-C₇ cycloalkyl;
each R⁶ is independently C₁-C₆ alkyl, and R⁷ is halo;
each R⁶ is independently C₁-C₆ alkyl, and R⁷ is cyano;
each R⁶ is independently C₃-C₇ cycloalkyl, and R⁷ is C₃-C₇ cycloalkyl;
each R⁶ is independently C₃-C₇ cycloalkyl, and R⁷ is halo;
each R⁶ is independently cyclopropyl and R⁷ is halo;
each R⁶ is independently C₁-C₆ alkyl, and R⁷ is C₁-C₆ alkoxy optionally substituted with one or more halo;
each R⁶ is independently C₁-C₆ alkyl, and R⁷ is C₁-C₆ alkoxy;
each R⁶ is independently C₁-C₆ alkyl, and R⁷ is C₁-C₆ alkoxy substituted with one or more halo;
each R⁶ is independently halo, and R⁷ is C₁-C₆ haloalkyl;
each R⁶ is independently halo, and R⁷ is C₁-C₆ haloalkoxy;
each R⁶ is independently C₁-C₆ alkoxy; and R⁷ is halo;
each R⁶ is independently C₁-C₆ alkoxy; and R⁷ is chloro;
R⁷ is C₁-C₆ alkyl, and each R⁶ is independently C₁-C₆ alkyl optionally substituted with one or more halo;
R⁷ is C₁-C₆ alkyl, and each R⁶ is independently C₁-C₆ alkyl substituted with one or more halo;
R⁷ is C₁-C₆ alkyl, and each R⁶ is independently C₃-C₇ cycloalkyl;
R⁷ is C₁-C₆ alkyl, and each R⁶ is independently halo;
R⁷ is C₁-C₆ alkyl and each R⁶ is independently halo;
R⁷ is C₁-C₆ alkyl, and each R⁶ is cyano;
R⁷ is C₃-C₇ cycloalkyl, and each R⁶ is independently C₃-C₇ cycloalkyl;
R⁷ is C₃-C₇ cycloalkyl, and each R⁶ is independently halo;
R⁷ is C₃-C₇ cycloalkyl and each R⁶ is independently halo;
R⁷ is C₁-C₆ alkyl, and each R⁶ is independently C₁-C₆ alkoxy optionally substituted with one or more halo;
R⁷ is C₁-C₆ alkyl, and each R⁶ is independently C₁-C₆ alkoxy;
R⁷ is C₁-C₆ alkyl, and each R⁶ is independently C₁-C₆ alkoxy substituted with one or more halo;
R⁷ is halo, and each R⁶ is independently C₁-C₆ haloalkyl;
R⁷ is halo, and each R⁶ is independently C₁-C₆ haloalkoxy;
R⁷ is C₁-C₆ alkoxy; and each R⁶ is independently halo;
R⁷ is C₁-C₆ alkoxy; and each R⁶ is chloro.

In some embodiments, of the compound of formula AA,

the substituted ring B is

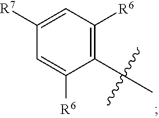

and R⁶ and R⁷ are one of the following combinations:
each R⁶ is isopropyl; and R⁷ is methyl;
each R⁶ is isopropyl; and R⁷ is isopropyl;
each R⁶ is isopropyl; and R⁷ is trifluoromethyl;
each R⁶ is isopropyl; and R⁷ is cyclopropyl;
each R⁶ is isopropyl; and R⁷ is chloro;
each R⁶ is isopropyl; and R⁷ is fluoro;
each R⁶ is ethyl; and R⁷ is fluoro;
each R⁶ is isopropyl; and R⁷ is cyano;
each R⁶ is cyclopropyl; and R⁷ is cyclopropyl;
each R⁶ is cyclopropyl; and R⁷ is chloro;
each R⁶ is cyclopropyl; and R⁷ is fluoro;
each R⁶ is isopropyl; and R⁷ is methoxy;
each R⁶ is isopropyl; and R⁷ is trifluoromethoxy;
each R⁶ is chloro; and R⁷ is trifluoromethyl;
each R⁶ is chloro; and R⁷ is trifluoromethoxy;
R⁷ is isopropyl; and each R⁶ is methyl;
R⁷ is isopropyl; and each R⁶ is trifluoromethyl;
R⁷ is isopropyl; and each R⁶ is cyclopropyl;
R⁷ is isopropyl; and each R⁶ is chloro;
R⁷ is ethyl; and each R⁶ is fluoro;
R⁷ is isopropyl; and each R⁶ is cyano;
R⁷ is cyclopropyl; and each R⁶ is cyclopropyl;
R⁷ is cyclopropyl; and each R⁶ is chloro;
R⁷ is cyclopropyl; and each R⁶ is fluoro;
R⁷ is isopropyl; and each R⁶ is methoxy;
R⁷ is isopropyl; and each R⁶ is trifluoromethoxy;
R⁷ is chloro; and each R⁶ is trifluoromethyl;
R⁷ is chloro; and each R⁶ is trifluoromethoxy;
one R⁶ is isopropyl; the other R⁶ is trifluoromethyl; and R⁷ is chloro.

In some embodiments, of the compound of formula AA,

the substituted ring B is

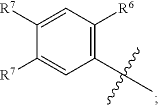

and R⁶ and R⁷ are one of the following combinations:
R⁶ is C₁-C₆ alkyl, and each R⁷ is independently C₁-C₆ alkyl optionally substituted with one or more halo;
R⁶ is C₁-C₆ alkyl and each R⁷ is independently C₁-C₆ alkyl;
R⁶ is C₁-C₆ alkyl, and each R⁷ is independently C₁-C₆ alkyl substituted with one or more halo;
R⁶ is C₁-C₆ alkyl, and each R⁷ is independently C₃-C₇ cycloalkyl;
R⁶ is C₁-C₆ alkyl, and each R⁷ is independently halo;
R⁶ is C₁-C₆ alkyl, and each R⁷ is cyano;
R⁶ is C₃-C₇ cycloalkyl, and each R⁷ is independently C₃-C₇ cycloalkyl;
R⁶ is C₃-C₇ cycloalkyl, and each R⁷ is independently halo;
R⁶ is cyclopropyl and each R⁷ is independently halo;
R⁶ is C₁-C₆ alkyl, and each R⁷ is independently C₁-C₆ alkoxy optionally substituted with one or more halo;
R⁶ is C₁-C₆ alkyl, and each R⁷ is independently C₁-C₆ alkoxy;
R⁶ is C₁-C₆ alkyl, and each R⁷ is independently C₁-C₆ alkoxy substituted with one or more halo;
R⁶ is halo, and each R⁷ is independently C₁-C₆ haloalkyl;
R⁶ is halo, and each R⁷ is independently C₁-C₆ haloalkoxy;
R⁶ is C₁-C₆ alkoxy; and each R⁷ is independently halo;
R⁶ is C₁-C₆ alkoxy; and each R⁷ is chloro;
each R⁷ is independently C₁-C₆ alkyl, and R⁶ is C₁-C₆ alkyl optionally substituted with one or more halo;
each R⁷ is independently C₁-C₆ alkyl, and R⁶ is C₁-C₆ alkyl substituted with one or more halo;
each R⁷ is independently C₁-C₆ alkyl, and R⁶ is C₃-C₇ cycloalkyl;
each R⁷ is independently C₁-C₆ alkyl, and R⁶ is halo;
each R⁷ is independently C₁-C₆ alkyl and R⁶ is halo;
each R⁷ is independently C₁-C₆ alkyl, and R⁶ is cyano;
each R⁷ is independently C₃-C₇ cycloalkyl, and R⁶ is C₃-C₇ cycloalkyl;
each R⁷ is independently C₃-C₇ cycloalkyl, and R⁶ is halo;
each R⁷ is independently C₃-C₇ cycloalkyl and R⁶ is halo;
each R⁷ is independently C₁-C₆ alkyl, and R⁶ is C₁-C₆ alkoxy optionally substituted with one or more halo;
each R⁷ is independently C₁-C₆ alkyl, and R⁶ is C₁-C₆ alkoxy;
each R⁷ is independently C₁-C₆ alkyl, and R⁶ is C₁-C₆ alkoxy substituted with one or more halo;
each R⁷ is independently halo, and R⁶ is C₁-C₆ haloalkyl;
each R⁷ is independently halo, and R⁶ is C₁-C₆ haloalkoxy;
each R⁷ is independently C₁-C₆ alkoxy; and R⁶ is halo;
each R⁷ is independently C₁-C₆ alkoxy; and R⁶ is chloro.

In some embodiments, of the compound of formula AA,

the substituted ring B is

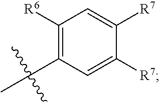

and R⁶ and R⁷ are one of the following combinations:
R⁶ is isopropyl; and each R⁷ is methyl;
R⁶ is isopropyl; and each R⁷ is isopropyl;
R⁶ is isopropyl; and each R⁷ is trifluoromethyl;
R⁶ is isopropyl; and each R⁷ is cyclopropyl;
R⁶ is isopropyl; and each R⁷ is chloro;
R⁶ is isopropyl; and each R⁷ is fluoro;
R⁶ is ethyl; and each R⁷ is fluoro;
R⁶ is isopropyl; and each R⁷ is cyano;
R⁶ is cyclopropyl; and each R⁷ is cyclopropyl;
R⁶ is cyclopropyl; and each R⁷ is chloro;
R⁶ is cyclopropyl; and each R⁷ is fluoro;
R⁶ is isopropyl; and each R⁷ is methoxy;
R⁶ is isopropyl; and each R⁷ is trifluoromethoxy;
R⁶ is chloro; and each R⁷ is trifluoromethyl;
R⁶ is chloro; and each R⁷ is trifluoromethoxy;
each R⁷ is isopropyl; and R⁶ is methyl;
each R⁷ is isopropyl; and R⁶ is trifluoromethyl;
each R⁷ is isopropyl; and R⁶ is cyclopropyl;
each R⁷ is isopropyl; and R⁶ is chloro;
each R⁷ is ethyl; and R⁶ is fluoro;
each R⁷ is isopropyl; and R⁶ is cyano;
each R⁷ is cyclopropyl; and R⁶ is cyclopropyl;
each R⁷ is cyclopropyl; and R⁶ is chloro;
each R⁷ is cyclopropyl; and R⁶ is fluoro;
each R⁷ is isopropyl; and R⁶ is methoxy;
each R⁷ is isopropyl; and R⁶ is trifluoromethoxy;
each R⁷ is chloro; and R⁶ is trifluoromethyl;
each R⁷ is chloro; and R⁶ is trifluoromethoxy.

In some embodiments, of the compound of formula AA,

the substituted ring B is

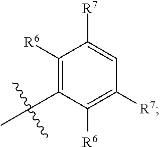

and R⁶ and R⁷ are one of the following combinations:
each R⁶ is independently C₁-C₆ alkyl, and each R⁷ is independently C₁-C₆ alkyl optionally substituted with one or more halo;
each R⁶ is independently C₁-C₆ alkyl and each R⁷ is independently C₁-C₆ alkyl;
each R⁶ is independently C₁-C₆ alkyl, and each R⁷ is independently C₁-C₆ alkyl substituted with one or more halo;
each R⁶ is independently C₁-C₆ alkyl, and each R⁷ is independently C₃-C₇ cycloalkyl;
each R⁶ is independently C₁-C₆ alkyl, and each R⁷ is independently halo;
each R⁶ is independently C₁-C₆ alkyl, and each R⁷ is cyano;
each R⁶ is independently C₃-C₇ cycloalkyl, and each R⁷ is independently C₃-C₇ cycloalkyl;
each R⁶ is independently C₃-C₇ cycloalkyl, and each R⁷ is independently halo;
each R⁶ is independently cyclopropyl and each R⁷ is independently halo;
each R⁶ is independently C₁-C₆ alkyl, and each R⁷ is independently C₁-C₆ alkoxy optionally substituted with one or more halo;
each R⁶ is independently C₁-C₆ alkyl, and each R⁷ is independently C₁-C₆ alkoxy;
each R⁶ is independently C₁-C₆ alkyl, and each R⁷ is independently C₁-C₆ alkoxy substituted with one or more halo;
each R⁶ is independently halo, and each R⁷ is independently C₁-C₆ haloalkyl;
each R⁶ is independently halo, and each R⁷ is independently C₁-C₆ haloalkoxy;
each R⁶ is independently C₁-C₆ alkoxy; and each R⁷ is independently halo;
each R⁶ is independently C₁-C₆ alkoxy; and each R⁷ is chloro;
each R⁷ is independently C₁-C₆ alkyl, and each R⁶ is independently C₁-C₆ alkyl optionally substituted with one or more halo;
each R⁷ is independently C₁-C₆ alkyl, and each R⁶ is independently C₁-C₆ alkyl substituted with one or more halo;
each R⁷ is independently C₁-C₆ alkyl, and each R⁶ is independently C₃-C₇ cycloalkyl;
each R⁷ is independently C₁-C₆ alkyl, and each R⁶ is independently halo;
each R⁷ is independently C₁-C₆ alkyl and each R⁶ is independently halo;
each R⁷ is independently C₁-C₆ alkyl, and each R⁶ is cyano;
each R⁷ is independently C₃-C₇ cycloalkyl, and each R⁶ is independently C₃-C₇ cycloalkyl;
each R⁷ is independently C₃-C₇ cycloalkyl, and each R⁶ is independently halo;
each R⁷ is independently C₃-C₇ cycloalkyl and each R⁶ is independently halo;
each R⁷ is independently C₁-C₆ alkyl, and each R⁶ is independently C₁-C₆ alkoxy optionally substituted with one or more halo;
each R⁷ is independently C₁-C₆ alkyl, and each R⁶ is independently C₁-C₆ alkoxy;
each R⁷ is independently C₁-C₆ alkyl, and each R⁶ is independently C₁-C₆ alkoxy substituted with one or more halo;
each R⁷ is independently halo, and each R⁶ is independently C₁-C₆ haloalkyl;
each R⁷ is independently halo, and each R⁶ is independently C₁-C₆ haloalkoxy;
each R⁷ is independently C₁-C₆ alkoxy; and each R⁶ is independently halo;
each R⁷ is independently C₁-C₆ alkoxy; and each R⁶ is chloro;
two pairs, each of one R⁶ and one R⁷, are on adjacent atoms, and each pair of one R⁶ and one R⁷ taken together with the atoms connecting them form a C₄-C₈ aliphatic carbocyclic ring;
two pairs, each of one R⁶ and one R⁷, are on adjacent atoms, and each pair of one R⁶ and one R⁷ taken together with the atoms connecting them form a C₄-C₆ aliphatic carbocyclic ring optionally substituted with one or more hydroxy, oxo, or C₁-C₆ alkyl; or
two pairs, each of one R⁶ and one R⁷, are on adjacent atoms, and each pair of one R⁶ and one R⁷ taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl.

In some embodiments, of the compound of formula AA,

the substituted ring B is

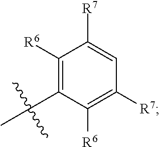

and R⁶ and R⁷ are one of the following combinations:
each R⁶ is isopropyl; and each R⁷ is methyl;
each R⁶ is isopropyl; and each R⁷ is isopropyl;
each R⁶ is isopropyl; and each R⁷ is trifluoromethyl;
each R⁶ is isopropyl; and each R⁷ is cyclopropyl;
each R⁶ is isopropyl; and each R⁷ is chloro;
each R⁶ is isopropyl; and each R⁷ is fluoro;
each R⁶ is ethyl; and each R⁷ is fluoro;
each R⁶ is isopropyl; and each R⁷ is cyano;
each R⁶ is cyclopropyl; and each R⁷ is cyclopropyl;
each R⁶ is cyclopropyl; and each R⁷ is chloro;
each R⁶ is cyclopropyl; and each R⁷ is fluoro;
each R⁶ is isopropyl; and each R⁷ is methoxy;
each R⁶ is isopropyl; and each R⁷ is trifluoromethoxy;
each R⁶ is chloro; and each R⁷ is trifluoromethyl;
each R⁶ is chloro; and each R⁷ is trifluoromethoxy;
each R⁷ is isopropyl; and each R⁶ is methyl;
each R⁷ is isopropyl; and each R⁶ is trifluoromethyl;
each R⁷ is isopropyl; and each R⁶ is cyclopropyl;
each R⁷ is isopropyl; and each R⁶ is chloro;
each R⁷ is ethyl; and each R⁶ is fluoro;
each R⁷ is isopropyl; and each R⁶ is cyano;
each R⁷ is cyclopropyl; and each R⁶ is cyclopropyl;
each R⁷ is cyclopropyl; and each R⁶ is chloro;
each R⁷ is cyclopropyl; and each R⁶ is fluoro;
each R⁷ is isopropyl; and each R⁶ is methoxy;
each R⁷ is isopropyl; and each R⁶ is trifluoromethoxy;
each R⁷ is chloro; and each R⁶ is trifluoromethyl;
each R⁷ is chloro; and each R⁶ is trifluoromethoxy;
one R⁶ is isopropyl; the other R⁶ is trifluoromethyl; and each R⁷ is chloro;
each R⁶ is isopropyl; one R⁷ is fluoro; and the other R⁷ is cyano;
two pairs, each of one R⁶ and one R⁷, are on adjacent atoms, and each pair of one R⁶ and one R⁷ taken together with the atoms connecting them form a C₅ aliphatic carbocyclic ring;
two pairs, each of one R⁶ and one R⁷, are on adjacent atoms, and each pair of one R⁶ and one R⁷ taken together with the atoms connecting them form a C₄ aliphatic carbocyclic ring optionally substituted with one or more hydroxy, oxo, or methyl;
two pairs, each of one R⁶ and one R⁷, are on adjacent atoms, and each pair of one R⁶ and one R⁷ taken together with the atoms connecting them form a C₅ aliphatic carbocyclic ring optionally substituted with one or more hydroxy, oxo, or methyl;
two pairs, each of one R⁶ and one R⁷, are on adjacent atoms, and each pair of one R⁶ and one R⁷ taken together with the atoms connecting them form a C₆ aliphatic carbocyclic ring optionally substituted with one or more hydroxy, oxo, or methyl;
two pairs, each of one R⁶ and one R⁷, are on adjacent atoms, and each pair of one R⁶ and one R⁷ taken together with the atoms connecting them form a 5-membered heterocyclic ring containing 1 heteroatom independently selected from O, N, and S, wherein the heterocyclic ring is optionally substituted with one or more hydroxy, oxo, or methyl; or
two pairs, each of one R⁶ and one R⁷, are on adjacent atoms, and each pair of one R⁶ and one R⁷ taken together with the atoms connecting them form a 6-membered heterocyclic ring containing 1 heteroatom independently selected from O, N, and S, wherein the heterocyclic ring is optionally substituted with one or more hydroxy, oxo, or methyl.

In some embodiments, of the compound of formula AA,

the substituted ring B is

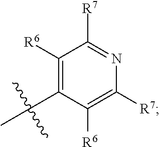

and R⁶ and R⁷ are one of the following combinations:
each R⁶ is independently C₁-C₆ alkyl, and each R⁷ is independently C₁-C₆ alkyl optionally substituted with one or more halo;
each R⁶ is independently C₁-C₆ alkyl and each R⁷ is independently C₁-C₆ alkyl;
each R⁶ is independently C₁-C₆ alkyl, and each R⁷ is independently C₁-C₆ alkyl substituted with one or more halo;
each R⁶ is independently C₁-C₆ alkyl, and each R⁷ is independently C₃-C₇ cycloalkyl;
each R⁶ is independently C₁-C₆ alkyl, and each R⁷ is independently halo;
each R⁶ is independently C₁-C₆ alkyl, and each R⁷ is cyano;
each R⁶ is independently C₃-C₇ cycloalkyl, and each R⁷ is independently C₃-C₇ cycloalkyl;
each R⁶ is independently C₃-C₇ cycloalkyl, and each R⁷ is independently halo;
each R⁶ is independently cyclopropyl and each R⁷ is independently halo;
each R⁶ is independently C₁-C₆ alkyl, and each R⁷ is independently C₁-C₆ alkoxy optionally substituted with one or more halo;
each R⁶ is independently C₁-C₆ alkyl, and each R⁷ is independently C₁-C₆ alkoxy;
each R⁶ is independently C₁-C₆ alkyl, and each R⁷ is independently C₁-C₆ alkoxy substituted with one or more halo;
each R⁶ is independently halo, and each R⁷ is independently C₁-C₆ haloalkyl;
each R⁶ is independently halo, and each R⁷ is independently C₁-C₆ haloalkoxy;
each R⁶ is independently C₁-C₆ alkoxy; and each R⁷ is independently halo;
each R⁶ is independently C₁-C₆ alkoxy; and each R⁷ is chloro;
each R⁷ is independently C₁-C₆ alkyl, and each R⁶ is independently C₁-C₆ alkyl optionally substituted with one or more halo;
each R⁷ is independently C₁-C₆ alkyl, and each R⁶ is independently C₁-C₆ alkyl substituted with one or more halo;
each R⁷ is independently C₁-C₆ alkyl, and each R⁶ is independently C₃-C₇ cycloalkyl;
each R⁷ is independently C₁-C₆ alkyl, and each R⁶ is independently halo;
each R⁷ is independently C₁-C₆ alkyl and each R⁶ is independently halo;
each R⁷ is independently C₁-C₆ alkyl, and each R⁶ is cyano;
each R⁷ is independently C₃-C₇ cycloalkyl, and each R⁶ is independently C₃-C₇ cycloalkyl;
each R⁷ is independently C₃-C₇ cycloalkyl, and each R⁶ is independently halo;
each R⁷ is independently C₃-C₇ cycloalkyl and each R⁶ is independently halo;
each R⁷ is independently C₁-C₆ alkyl, and each R⁶ is independently C₁-C₆ alkoxy optionally substituted with one or more halo;
each R⁷ is independently C₁-C₆ alkyl, and each R⁶ is independently C₁-C₆ alkoxy;
each R⁷ is independently C₁-C₆ alkyl, and each R⁶ is independently C₁-C₆ alkoxy substituted with one or more halo;
each R⁷ is independently halo, and each R⁶ is independently C₁-C₆ haloalkyl;
each R⁷ is independently halo, and each R⁶ is independently C₁-C₆ haloalkoxy;
each R⁷ is independently C₁-C₆ alkoxy; and each R⁶ is independently halo;
each R⁷ is independently C₁-C₆ alkoxy; and each R⁶ is chloro;
two pairs, each of one R⁶ and one R⁷, are on adjacent atoms, and each pair of one R⁶ and one R⁷ taken together with the atoms connecting them form a C₄-C₈ aliphatic carbocyclic ring;
two pairs, each of one R⁶ and one R⁷, are on adjacent atoms, and each pair of one R⁶ and one R⁷ taken together with the atoms connecting them form a C₄-C₆ aliphatic carbocyclic ring optionally substituted with one or more hydroxy, oxo, or C₁-C₆ alkyl; or
two pairs, each of one R⁶ and one R⁷, are on adjacent atoms, and each pair of one R⁶ and one R⁷ taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl.

In some embodiments, of the compound of formula AA,

the substituted ring B is

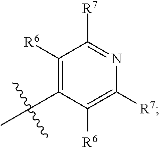

and R⁶ and R⁷ are one of the following combinations:
each R⁶ is isopropyl; and each R⁷ is methyl;
each R⁶ is isopropyl; and each R⁷ is isopropyl;
each R⁶ is isopropyl; and each R⁷ is trifluoromethyl;
each R⁶ is isopropyl; and each R⁷ is cyclopropyl;
each R⁶ is isopropyl; and each R⁷ is chloro;
each R⁶ is isopropyl; and each R⁷ is fluoro;
each R⁶ is ethyl; and each R⁷ is fluoro;
each R⁶ is isopropyl; and each R⁷ is cyano;
each R⁶ is cyclopropyl; and each R⁷ is cyclopropyl;
each R⁶ is cyclopropyl; and each R⁷ is chloro;
each R⁶ is cyclopropyl; and each R⁷ is fluoro;
each R⁶ is isopropyl; and each R⁷ is methoxy;
each R⁶ is isopropyl; and each R⁷ is trifluoromethoxy;
each R⁶ is chloro; and each R⁷ is trifluoromethyl;
each R⁶ is chloro; and each R⁷ is trifluoromethoxy;
each R⁷ is isopropyl; and each R⁶ is methyl;
each R⁷ is isopropyl; and each R⁶ is trifluoromethyl;
each R⁷ is isopropyl; and each R⁶ is cyclopropyl;
each R⁷ is isopropyl; and each R⁶ is chloro;
each R⁷ is ethyl; and each R⁶ is fluoro;
each R⁷ is isopropyl; and each R⁶ is cyano;
each R⁷ is cyclopropyl; and each R⁶ is cyclopropyl;
each R⁷ is cyclopropyl; and each R⁶ is chloro;
each R⁷ is cyclopropyl; and each R⁶ is fluoro;
each R⁷ is isopropyl; and each R⁶ is methoxy;
each R⁷ is isopropyl; and each R⁶ is trifluoromethoxy;
each R⁷ is chloro; and each R⁶ is trifluoromethyl;
each R⁷ is chloro; and each R⁶ is trifluoromethoxy;
one R⁶ is isopropyl; the other R⁶ is trifluoromethyl; and each R⁷ is chloro;
each R⁶ is isopropyl; one R⁷ is fluoro; and the other R⁷ is cyano;
two pairs, each of one R⁶ and one R⁷, are on adjacent atoms, and each pair of one R⁶ and one R⁷ taken together with the atoms connecting them form a C₅ aliphatic carbocyclic ring;
two pairs, each of one R⁶ and one R⁷, are on adjacent atoms, and each pair of one R⁶ and one R⁷ taken together with the atoms connecting them form a C₄ aliphatic carbocyclic ring optionally substituted with one or more hydroxy, oxo, or methyl;
two pairs, each of one R⁶ and one R⁷, are on adjacent atoms, and each pair of one R⁶ and one R⁷ taken together with the atoms connecting them form a C₅ aliphatic carbocyclic ring optionally substituted with one or more hydroxy, oxo, or methyl;
two pairs, each of one R⁶ and one R⁷, are on adjacent atoms, and each pair of one R⁶ and one R⁷ taken together with the atoms connecting them form a C₆ aliphatic carbocyclic ring optionally substituted with one or more hydroxy, oxo, or methyl;
two pairs, each of one R⁶ and one R⁷, are on adjacent atoms, and each pair of one R⁶ and one R⁷ taken together with the atoms connecting them form a 5-membered heterocyclic ring containing 1 heteroatom independently selected from O, N, and S, wherein the heterocyclic ring is optionally substituted with one or more hydroxy, oxo, or methyl; or
two pairs, each of one R⁶ and one R⁷, are on adjacent atoms, and each pair of one R⁶ and one R⁷ taken together with the atoms connecting them form a 6-membered heterocyclic ring containing 1 heteroatom independently selected from O, N, and S, wherein the heterocyclic ring is optionally substituted with one or more hydroxy, oxo, or methyl.

In some embodiments, of the compound of formula AA,

the substituted ring B is

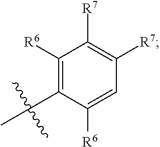

and R⁶ and R⁷ are one of the following combinations:
each R⁶ is independently C₁-C₆ alkyl, and each R⁷ is independently C₁-C₆ alkyl optionally substituted with one or more halo;
each R⁶ is independently C₁-C₆ alkyl and each R⁷ is independently C₁-C₆ alkyl;
each R⁶ is independently C₁-C₆ alkyl, and each R⁷ is independently C₁-C₆ alkyl substituted with one or more halo;
each R⁶ is independently C₁-C₆ alkyl, and each R⁷ is independently C₃-C₇ cycloalkyl;
each R⁶ is independently C₁-C₆ alkyl, and each R⁷ is independently halo;
each R⁶ is independently C₁-C₆ alkyl, and each R⁷ is cyano;
each R⁶ is independently C₃-C₇ cycloalkyl, and each R⁷ is independently C₃-C₇ cycloalkyl;
each R⁶ is independently C₃-C₇ cycloalkyl, and each R⁷ is independently halo;
each R⁶ is independently cyclopropyl and each R⁷ is independently halo;
each R⁶ is independently C₁-C₆ alkyl, and each R⁷ is independently C₁-C₆ alkoxy optionally substituted with one or more halo;
each R⁶ is independently C₁-C₆ alkyl, and each R⁷ is independently C₁-C₆ alkoxy;
each R⁶ is independently C₁-C₆ alkyl, and each R⁷ is independently C₁-C₆ alkoxy substituted with one or more halo;
each R⁶ is independently halo, and each R⁷ is independently C₁-C₆ haloalkyl;
each R⁶ is independently halo, and each R⁷ is independently C₁-C₆ haloalkoxy;
each R⁶ is independently C₁-C₆ alkoxy; and each R⁷ is independently halo;
each R⁶ is independently C₁-C₆ alkoxy; and each R⁷ is chloro;
each R⁷ is independently C₁-C₆ alkyl, and each R⁶ is independently C₁-C₆ alkyl optionally substituted with one or more halo;
each R⁷ is independently C₁-C₆ alkyl, and each R⁶ is independently C₁-C₆ alkyl substituted with one or more halo;
each R⁷ is independently C₁-C₆ alkyl, and each R⁶ is independently C₃-C₇ cycloalkyl;
each R⁷ is independently C₁-C₆ alkyl, and each R⁶ is independently halo;
each R⁷ is independently C₁-C₆ alkyl and each R⁶ is independently halo;
each R⁷ is independently C₁-C₆ alkyl, and each R⁶ is cyano;
each R⁷ is independently C₃-C₇ cycloalkyl, and each R⁶ is independently C₃-C₇ cycloalkyl;
each R⁷ is independently C₃-C₇ cycloalkyl, and each R⁶ is independently halo;
each R⁷ is independently C₃-C₇ cycloalkyl and each R⁶ is independently halo;
each R⁷ is independently C₁-C₆ alkyl, and each R⁶ is independently C₁-C₆ alkoxy optionally substituted with one or more halo;
each R⁷ is independently C₁-C₆ alkyl, and each R⁶ is independently C₁-C₆ alkoxy;
each R⁷ is independently C₁-C₆ alkyl, and each R⁶ is independently C₁-C₆ alkoxy substituted with one or more halo;
each R⁷ is independently halo, and each R⁶ is independently C₁-C₆ haloalkyl;
each R⁷ is independently halo, and each R⁶ is independently C₁-C₆ haloalkoxy;
each R⁷ is independently C₁-C₆ alkoxy; and each R⁶ is independently halo;
each R⁷ is independently C₁-C₆ alkoxy; and each R⁶ is chloro.

In some embodiments, of the compound of formula AA,

the substituted ring B is

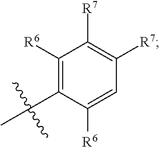

and R⁶ and R⁷ are one of the following combinations:
each R⁶ is isopropyl; and each R⁷ is methyl;
each R⁶ is isopropyl; and each R⁷ is isopropyl;
each R⁶ is isopropyl; and each R⁷ is trifluoromethyl;
each R⁶ is isopropyl; and each R⁷ is cyclopropyl;
each R⁶ is isopropyl; and each R⁷ is chloro;
each R⁶ is isopropyl; and each R⁷ is fluoro;
each R⁶ is ethyl; and each R⁷ is fluoro;
each R⁶ is isopropyl; and each R⁷ is cyano;
each R⁶ is cyclopropyl; and each R⁷ is cyclopropyl;
each R⁶ is cyclopropyl; and each R⁷ is chloro;
each R⁶ is cyclopropyl; and each R⁷ is fluoro;
each R⁶ is isopropyl; and each R⁷ is methoxy;
each R⁶ is isopropyl; and each R⁷ is trifluoromethoxy;
each R⁶ is chloro; and each R⁷ is trifluoromethyl;
each R⁶ is chloro; and each R⁷ is trifluoromethoxy;
each R⁷ is isopropyl; and each R⁶ is methyl;
each R⁷ is isopropyl; and each R⁶ is trifluoromethyl;
each R⁷ is isopropyl; and each R⁶ is cyclopropyl;
each R⁷ is isopropyl; and each R⁶ is chloro;
each R⁷ is ethyl; and each R⁶ is fluoro;
each R⁷ is isopropyl; and each R⁶ is cyano;
each R⁷ is cyclopropyl; and each R⁶ is cyclopropyl;
each R⁷ is cyclopropyl; and each R⁶ is chloro;
each R⁷ is cyclopropyl; and each R⁶ is fluoro;
each R⁷ is isopropyl; and each R⁶ is methoxy;
each R⁷ is isopropyl; and each R⁶ is trifluoromethoxy;
each R⁷ is chloro; and each R⁶ is trifluoromethyl;
each R⁷ is chloro; and each R⁶ is trifluoromethoxy;
one R⁶ is isopropyl; the other R⁶ is trifluoromethyl; and each R⁷ is chloro;
each R⁶ is isopropyl; one R⁷ is fluoro; and the other R⁷ is cyano.

In some embodiments, of the compound of formula AA,

the substituted ring B is

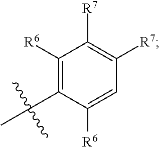

and R⁶ and R⁷ are one of the following combinations:
each R⁶ is independently C₁-C₆ alkyl, and each R⁷ is independently C₁-C₆ alkyl optionally substituted with one or more halo;
each R⁶ is independently C₁-C₆ alkyl and each R⁷ is independently C₁-C₆ alkyl;
each R⁶ is independently C₁-C₆ alkyl, and each R⁷ is independently C₁-C₆ alkyl substituted with one or more halo;
each R⁶ is independently C₁-C₆ alkyl, and each R⁷ is independently C₃-C₇ cycloalkyl;
each R⁶ is independently C₁-C₆ alkyl, and each R⁷ is independently halo;
each R⁶ is independently C₁-C₆ alkyl, and each R⁷ is cyano;
each R⁶ is independently C₃-C₇ cycloalkyl, and each R⁷ is independently C₃-C₇ cycloalkyl;
each R⁶ is independently C₃-C₇ cycloalkyl, and each R⁷ is independently halo;
each R⁶ is independently cyclopropyl and each R⁷ is independently halo;
each R⁶ is independently C₁-C₆ alkyl, and each R⁷ is independently C₁-C₆ alkoxy optionally substituted with one or more halo;
each R⁶ is independently C₁-C₆ alkyl, and each R⁷ is independently C₁-C₆ alkoxy;
each R⁶ is independently C₁-C₆ alkyl, and each R⁷ is independently C₁-C₆ alkoxy substituted with one or more halo;
each R⁶ is independently halo, and each R⁷ is independently C₁-C₆ haloalkyl;
each R⁶ is independently halo, and each R⁷ is independently C₁-C₆ haloalkoxy;
each R⁶ is independently C₁-C₆ alkoxy; and each R⁷ is independently halo;
each R⁶ is independently C₁-C₆ alkoxy; and each R⁷ is chloro;
each R⁷ is independently C₁-C₆ alkyl, and each R⁶ is independently C₁-C₆ alkyl optionally substituted with one or more halo;
each R⁷ is independently C₁-C₆ alkyl, and each R⁶ is independently C₁-C₆ alkyl substituted with one or more halo;
each R⁷ is independently C₁-C₆ alkyl, and each R⁶ is independently C₃-C₇ cycloalkyl;
each R⁷ is independently C₁-C₆ alkyl, and each R⁶ is independently halo;
each R⁷ is independently C₁-C₆ alkyl and each R⁶ is independently halo;
each R⁷ is independently C₁-C₆ alkyl, and each R⁶ is cyano;
each R⁷ is independently C₃-C₇ cycloalkyl, and each R⁶ is independently C₃-C₇ cycloalkyl;
each R⁷ is independently C₃-C₇ cycloalkyl, and each R⁶ is independently halo;
each R⁷ is independently C₃-C₇ cycloalkyl and each R⁶ is independently halo;
each R⁷ is independently C₁-C₆ alkyl, and each R⁶ is independently C₁-C₆ alkoxy optionally substituted with one or more halo;
each R⁷ is independently C₁-C₆ alkyl, and each R⁶ is independently C₁-C₆ alkoxy;
each R⁷ is independently C₁-C₆ alkyl, and each R⁶ is independently C₁-C₆ alkoxy substituted with one or more halo;
each R⁷ is independently halo, and each R⁶ is independently C₁-C₆ haloalkyl;
each R⁷ is independently halo, and each R⁶ is independently C₁-C₆ haloalkoxy;
each R⁷ is independently C₁-C₆ alkoxy; and each R⁶ is independently halo;
each R⁷ is independently C₁-C₆ alkoxy; and each R⁶ is chloro;
R⁶ and R⁷ on adjacent atoms taken together with the atoms connecting them form a C₄-C₆ aliphatic carbocyclic ring optionally substituted with one or more hydroxy, oxo, or C₁-C₆ alkyl; and one R⁶ is halo or cyano; or
R⁶ and R⁷ on adjacent atoms taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl; and one R⁶ is halo or cyano.

In some embodiments, of the compound of formula AA,

the substituted ring B is

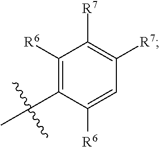

and R⁶ and R⁷ are one of the following combinations:
each R⁶ is isopropyl; and each R⁷ is methyl;
each R⁶ is isopropyl; and each R⁷ is isopropyl;
each R⁶ is isopropyl; and each R⁷ is trifluoromethyl;
each R⁶ is isopropyl; and each R⁷ is cyclopropyl;
each R⁶ is isopropyl; and each R⁷ is chloro;
each R⁶ is isopropyl; and each R⁷ is fluoro;
each R⁶ is ethyl; and each R⁷ is fluoro;
each R⁶ is isopropyl; and each R⁷ is cyano;
each R⁶ is cyclopropyl; and each R⁷ is cyclopropyl;
each R⁶ is cyclopropyl; and each R⁷ is chloro;
each R⁶ is cyclopropyl; and each R⁷ is fluoro;
each R⁶ is isopropyl; and each R⁷ is methoxy;
each R⁶ is isopropyl; and each R⁷ is trifluoromethoxy;
each R⁶ is chloro; and each R⁷ is trifluoromethyl;
each R⁶ is chloro; and each R⁷ is trifluoromethoxy;
each R⁷ is isopropyl; and each R⁶ is methyl;
each R⁷ is isopropyl; and each R⁶ is trifluoromethyl;
each R⁷ is isopropyl; and each R⁶ is cyclopropyl;
each R⁷ is isopropyl; and each R⁶ is chloro;
each R⁷ is ethyl; and each R⁶ is fluoro;
each R⁷ is isopropyl; and each R⁶ is cyano;
each R⁷ is cyclopropyl; and each R⁶ is cyclopropyl;
each R⁷ is cyclopropyl; and each R⁶ is chloro;
each R⁷ is cyclopropyl; and each R⁶ is fluoro;
each R⁷ is isopropyl; and each R⁶ is methoxy;
each R⁷ is isopropyl; and each R⁶ is trifluoromethoxy;
each R⁷ is chloro; and each R⁶ is trifluoromethyl;
each R⁷ is chloro; and each R⁶ is trifluoromethoxy;
one R⁶ is isopropyl; the other R⁶ is trifluoromethyl; and each R⁷ is chloro;
each R⁶ is isopropyl; one R⁷ is fluoro; and the other R⁷ is cyano;
R⁶ and R⁷ on adjacent atoms taken together with the atoms connecting them form a C₄ aliphatic carbocyclic ring; and one R⁶ is chloro, fluoro, or cyano;
R⁶ and R⁷ on adjacent atoms taken together with the atoms connecting them form a C₅ aliphatic carbocyclic ring; and one R⁶ is chloro, fluoro, or cyano;
R⁶ and R⁷ on adjacent atoms taken together with the atoms connecting them form a C₆ aliphatic carbocyclic ring; and one R⁶ is chloro, fluoro, or cyano;
R⁶ and R⁷ on adjacent atoms taken together with the atoms connecting them form a 5-membered heterocyclic ring containing 1 heteroatoms independently selected from O, N, and S; and one R⁶ is chloro, fluoro, or cyano;
R⁶ and R⁷ on adjacent atoms taken together with the atoms connecting them form a 6-membered heterocyclic ring containing 1 heteroatoms independently selected from O, N, and S; and one R⁶ is chloro, fluoro, or cyano; or
R⁶ and R⁷ on adjacent atoms taken together with the atoms connecting them form a C₅ aliphatic carbocyclic ring; and one R⁶ is chloro, fluoro, or cyano.

In some embodiments, of the compound of formula AA,

the substituted ring B is

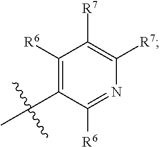

and R⁶ and R⁷ are one of the following combinations:
each R⁶ is independently C₁-C₆ alkyl, and each R⁷ is independently C₁-C₆ alkyl optionally substituted with one or more halo;
each R⁶ is independently C₁-C₆ alkyl and each R⁷ is independently C₁-C₆ alkyl;
each R⁶ is independently C₁-C₆ alkyl, and each R⁷ is independently C₁-C₆ alkyl substituted with one or more halo;
each R⁶ is independently C₁-C₆ alkyl, and each R⁷ is independently C₃-C₇ cycloalkyl;
each R⁶ is independently C₁-C₆ alkyl, and each R⁷ is independently halo;
each R⁶ is independently C₁-C₆ alkyl, and each R⁷ is cyano;
each R⁶ is independently C₃-C₇ cycloalkyl, and each R⁷ is independently C₃-C₇ cycloalkyl;
each R⁶ is independently C₃-C₇ cycloalkyl, and each R⁷ is independently halo;
each R⁶ is independently cyclopropyl and each R⁷ is independently halo;
each R⁶ is independently C₁-C₆ alkyl, and each R⁷ is independently C₁-C₆ alkoxy optionally substituted with one or more halo;
each R⁶ is independently C₁-C₆ alkyl, and each R⁷ is independently C₁-C₆ alkoxy;
each R⁶ is independently C₁-C₆ alkyl, and each R⁷ is independently C₁-C₆ alkoxy substituted with one or more halo;
each R⁶ is independently halo, and each R⁷ is independently C₁-C₆ haloalkyl;
each R⁶ is independently halo, and each R⁷ is independently C₁-C₆ haloalkoxy;
each R⁶ is independently C₁-C₆ alkoxy; and each R⁷ is independently halo;
each R⁶ is independently C₁-C₆ alkoxy; and R⁷ is chloro;
each R⁷ is independently C₁-C₆ alkyl, and each R⁶ is independently C₁-C₆ alkyl optionally substituted with one or more halo;
each R⁷ is independently C₁-C₆ alkyl, and each R⁶ is independently C₁-C₆ alkyl substituted with one or more halo;
each R⁷ is independently C₁-C₆ alkyl, and each R⁶ is independently C₃-C₇ cycloalkyl;
each R⁷ is independently C₁-C₆ alkyl, and each R⁶ is independently halo;
each R⁷ is independently C₁-C₆ alkyl and each R⁶ is independently halo;
each R⁷ is independently C₁-C₆ alkyl, and R⁶ is cyano;
each R⁷ is independently C₃-C₇ cycloalkyl, and each R⁶ is independently C₃-C₇ cycloalkyl;
each R⁷ is independently C₃-C₇ cycloalkyl, and each R⁶ is independently halo;
each R⁷ is independently C₃-C₇ cycloalkyl and each R⁶ is independently halo;
each R⁷ is independently C₁-C₆ alkyl, and each R⁶ is independently C₁-C₆ alkoxy optionally substituted with one or more halo;
each R⁷ is independently C₁-C₆ alkyl, and each R⁶ is independently C₁-C₆ alkoxy;
each R⁷ is independently C₁-C₆ alkyl, and each R⁶ is independently C₁-C₆ alkoxy substituted with one or more halo;
each R⁷ is independently halo, and each R⁶ is independently C₁-C₆ haloalkyl;
each R⁷ is independently halo, and each R⁶ is independently C₁-C₆ haloalkoxy;
each R⁷ is independently C₁-C₆ alkoxy; and each R⁶ is independently halo;
each R⁷ is independently C₁-C₆ alkoxy; and R⁶ is chloro.

In some embodiments, of the compound of formula AA,

the substituted ring B is

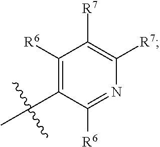

and R⁶ and R⁷ are one of the following combinations:
each R⁶ is isopropyl; and each R⁷ is methyl;
each R⁶ is isopropyl; and each R⁷ is isopropyl;
each R⁶ is isopropyl; and each R⁷ is trifluoromethyl;
each R⁶ is isopropyl; and each R⁷ is cyclopropyl;
each R⁶ is isopropyl; and each R⁷ is chloro;
each R⁶ is isopropyl; and each R⁷ is fluoro;
each R⁶ is ethyl; and each R⁷ is fluoro;
each R⁶ is isopropyl; and each R⁷ is cyano;
each R⁶ is cyclopropyl; and each R⁷ is cyclopropyl;
each R⁶ is cyclopropyl; and each R⁷ is chloro;
each R⁶ is cyclopropyl; and each R⁷ is fluoro;
each R⁶ is isopropyl; and each R⁷ is methoxy;
each R⁶ is isopropyl; and each R⁷ is trifluoromethoxy;
each R⁶ is chloro; and each R⁷ is trifluoromethyl;
each R⁶ is chloro; and each R⁷ is trifluoromethoxy;
each R⁷ is isopropyl; and each R⁶ is methyl;
each R⁷ is isopropyl; and each R⁶ is trifluoromethyl;
each R⁷ is isopropyl; and each R⁶ is cyclopropyl;
each R⁷ is isopropyl; and each R⁶ is chloro;
each R⁷ is ethyl; and each R⁶ is fluoro;
each R⁷ is isopropyl; and each R⁶ is cyano;
each R⁷ is cyclopropyl; and each R⁶ is cyclopropyl;
each R⁷ is cyclopropyl; and each R⁶ is chloro;
each R⁷ is cyclopropyl; and each R⁶ is fluoro;
each R⁷ is isopropyl; and each R⁶ is methoxy;
each R⁷ is isopropyl; and each R⁶ is trifluoromethoxy;
each R⁷ is chloro; and each R⁶ is trifluoromethyl;
each R⁷ is chloro; and each R⁶ is trifluoromethoxy;
one R⁶ is isopropyl; the other R⁶ is trifluoromethyl; and each R⁷ is chloro;
each R⁶ is isopropyl; one R⁷ is fluoro; and the other R⁷ is cyano.

In some embodiments, of the compound of formula AA,

the substituted ring B is

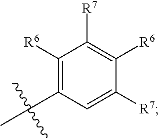

and R⁶ and R⁷ are one of the following combinations:
each R⁶ is independently C₁-C₆ alkyl, and each R⁷ is independently C₁-C₆ alkyl optionally substituted with one or more halo;
each R⁶ is independently C₁-C₆ alkyl and each R⁷ is independently C₁-C₆ alkyl;
each R⁶ is independently C₁-C₆ alkyl, and each R⁷ is independently C₁-C₆ alkyl substituted with one or more halo;
each R⁶ is independently C₁-C₆ alkyl, and each R⁷ is independently C₃-C₇ cycloalkyl;
each R⁶ is independently C₁-C₆ alkyl, and each R⁷ is independently halo;
each R⁶ is independently C₁-C₆ alkyl, and each R⁷ is cyano;
each R⁶ is independently C₃-C₇ cycloalkyl, and each R⁷ is independently C₃-C₇ cycloalkyl;
each R⁶ is independently C₃-C₇ cycloalkyl, and each R⁷ is independently halo;
each R⁶ is independently cyclopropyl and each R⁷ is independently halo;
each R⁶ is independently C₁-C₆ alkyl, and each R⁷ is independently C₁-C₆ alkoxy optionally substituted with one or more halo;
each R⁶ is independently C₁-C₆ alkyl, and each R⁷ is independently C₁-C₆ alkoxy;
each R⁶ is independently C₁-C₆ alkyl, and each R⁷ is independently C₁-C₆ alkoxy substituted with one or more halo;
each R⁶ is independently halo, and each R⁷ is independently C₁-C₆ haloalkyl;
each R⁶ is independently halo, and each R⁷ is independently C₁-C₆ haloalkoxy;
each R⁶ is independently C₁-C₆ alkoxy; and each R⁷ is independently halo;
each R⁶ is independently C₁-C₆ alkoxy; and each R⁷ is chloro;
each R⁷ is independently C₁-C₆ alkyl, and each R⁶ is independently C₁-C₆ alkyl optionally substituted with one or more halo;
each R⁷ is independently C₁-C₆ alkyl, and each R⁶ is independently C₁-C₆ alkyl substituted with one or more halo;
each R⁷ is independently C₁-C₆ alkyl, and each R⁶ is independently C₃-C₇ cycloalkyl;
each R⁷ is independently C₁-C₆ alkyl, and each R⁶ is independently halo;
each R⁷ is independently C₁-C₆ alkyl and each R⁶ is independently halo;
each R⁷ is independently C₁-C₆ alkyl, and each R⁶ is cyano;
each R⁷ is independently C₃-C₇ cycloalkyl, and each R⁶ is independently C₃-C₇ cycloalkyl;
each R⁷ is independently C₃-C₇ cycloalkyl, and each R⁶ is independently halo;
each R⁷ is independently C₃-C₇ cycloalkyl and each R⁶ is independently halo;
each R⁷ is independently C₁-C₆ alkyl, and each R⁶ is independently C₁-C₆ alkoxy optionally substituted with one or more halo;
each R⁷ is independently C₁-C₆ alkyl, and each R⁶ is independently C₁-C₆ alkoxy;
each R⁷ is independently C₁-C₆ alkyl, and each R⁶ is independently C₁-C₆ alkoxy substituted with one or more halo;
each R⁷ is independently halo, and each R⁶ is independently C₁-C₆ haloalkyl;
each R⁷ is independently halo, and each R⁶ is independently C₁-C₆ haloalkoxy;
each R⁷ is independently C₁-C₆ alkoxy; and each R⁶ is independently halo;
each R⁷ is independently C₁-C₆ alkoxy; and each R⁶ is chloro;
Two pairs, each of one R⁶ and one R⁷, are on adjacent atoms, and each pair of one R⁶ and one R⁷ taken together with the atoms connecting them form a C₄-C₈ aliphatic carbocyclic ring;
R⁶ and R⁷ on adjacent atoms taken together with the atoms connecting them form a C₄-C₆ aliphatic carbocyclic ring optionally substituted with one or more hydroxy, oxo, or C₁-C₆ alkyl; and one R⁶ is halo or cyano; or
R⁶ and R⁷ on adjacent atoms taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl; and one R⁶ is halo or cyano.

In some embodiments, of the compound of formula AA,

the substituted ring B is

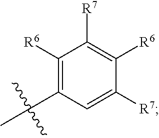

and R⁶ and R⁷ are one of the following combinations:
each R⁶ is isopropyl; and each R⁷ is methyl;
each R⁶ is isopropyl; and each R⁷ is isopropyl;
each R⁶ is isopropyl; and each R⁷ is trifluoromethyl;
each R⁶ is isopropyl; and each R⁷ is cyclopropyl;
each R⁶ is isopropyl; and each R⁷ is chloro;
each R⁶ is isopropyl; and each R⁷ is fluoro;
each R⁶ is ethyl; and each R⁷ is fluoro;
each R⁶ is isopropyl; and each R⁷ is cyano;
each R⁶ is cyclopropyl; and each R⁷ is cyclopropyl;
each R⁶ is cyclopropyl; and each R⁷ is chloro;
each R⁶ is cyclopropyl; and each R⁷ is fluoro;
each R⁶ is isopropyl; and each R⁷ is methoxy;
each R⁶ is isopropyl; and each R⁷ is trifluoromethoxy;
each R⁶ is chloro; and each R⁷ is trifluoromethyl;
each R⁶ is chloro; and each R⁷ is trifluoromethoxy;
each R⁷ is isopropyl; and each R⁶ is methyl;
each R⁷ is isopropyl; and each R⁶ is trifluoromethyl;
each R⁷ is isopropyl; and each R⁶ is cyclopropyl;
each R⁷ is isopropyl; and each R⁶ is chloro;
each R⁷ is ethyl; and each R⁶ is fluoro;
each R⁷ is isopropyl; and each R⁶ is cyano;
each R⁷ is cyclopropyl; and each R⁶ is cyclopropyl;
each R⁷ is cyclopropyl; and each R⁶ is chloro;
each R⁷ is cyclopropyl; and each R⁶ is fluoro;
each R⁷ is isopropyl; and each R⁶ is methoxy;
each R⁷ is isopropyl; and each R⁶ is trifluoromethoxy;
each R⁷ is chloro; and each R⁶ is trifluoromethyl;
each R⁷ is chloro; and each R⁶ is trifluoromethoxy;
one R⁶ is isopropyl; the other R⁶ is trifluoromethyl; and each R⁷ is chloro;
each R⁶ is isopropyl; one R⁷ is fluoro; and the other R⁷ is cyano;
two pairs, each of one R⁶ and one R⁷, are on adjacent atoms, and each pair of one R⁶ and one R⁷ taken together with the atoms connecting them form a C₅ aliphatic carbocyclic ring;
R⁶ and R⁷ on adjacent atoms taken together with the atoms connecting them form a C₄ aliphatic carbocyclic ring; and one R⁶ is chloro, fluoro, or cyano;
R⁶ and R⁷ on adjacent atoms taken together with the atoms connecting them form a C₅ aliphatic carbocyclic ring; and one R⁶ is chloro, fluoro, or cyano;
R⁶ and R⁷ on adjacent atoms taken together with the atoms connecting them form a C₆ aliphatic carbocyclic ring; and one R⁶ is chloro, fluoro, or cyano;
R⁶ and R⁷ on adjacent atoms taken together with the atoms connecting them form a 5-membered heterocyclic ring containing 1 heteroatoms independently selected from O, N, and S; and one R⁶ is chloro, fluoro, or cyano;
R⁶ and R⁷ on adjacent atoms taken together with the atoms connecting them form a 6-membered heterocyclic ring containing 1 heteroatoms independently selected from O, N, and S; and one R⁶ is chloro, fluoro, or cyano; or
R⁶ and R⁷ on adjacent atoms taken together with the atoms connecting them form a C₅ aliphatic carbocyclic ring; and one R⁶ is chloro, fluoro, or cyano.

In some embodiments, of the compound of formula AA,

the substituted ring B is

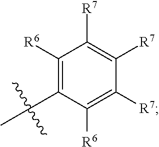

and R⁶ and R⁷ are one of the following combinations:
each R⁶ is independently C₁-C₆ alkyl, and each R⁷ is C₁-C₆ alkyl optionally substituted with one or more halo;
each R⁶ is independently C₁-C₆ alkyl and R⁷ is C₁-C₆ alkyl;
each R⁶ is independently C₁-C₆ alkyl, and R⁷ is C₁-C₆ alkyl substituted with one or more halo;
each R⁶ is independently C₁-C₆ alkyl, and R⁷ is C₃-C₇ cycloalkyl;
each R⁶ is independently C₁-C₆ alkyl, and R⁷ is halo;
each R⁶ is independently C₁-C₆ alkyl, and R⁷ is cyano;
each R⁶ is independently C₃-C₇ cycloalkyl, and R⁷ is C₃-C₇ cycloalkyl;
each R⁶ is independently C₃-C₇ cycloalkyl, and R⁷ is halo;
each R⁶ is independently cyclopropyl and R⁷ is halo;
each R⁶ is independently C₁-C₆ alkyl, and R⁷ is C₁-C₆ alkoxy optionally substituted with one or more halo;
each R⁶ is independently C₁-C₆ alkyl, and R⁷ is C₁-C₆ alkoxy;
each R⁶ is independently C₁-C₆ alkyl, and R⁷ is C₁-C₆ alkoxy substituted with one or more halo;
each R⁶ is independently halo, and R⁷ is C₁-C₆ haloalkyl;
each R⁶ is independently halo, and R⁷ is C₁-C₆ haloalkoxy;
each R⁶ is independently C₁-C₆ alkoxy; and R⁷ is halo;
each R⁶ is independently C₁-C₆ alkoxy; and R⁷ is chloro;
R⁷ is C₁-C₆ alkyl, and each R⁶ is independently C₁-C₆ alkyl optionally substituted with one or more halo;
R⁷ is C₁-C₆ alkyl, and each R⁶ is independently C₁-C₆ alkyl substituted with one or more halo;
R⁷ is C₁-C₆ alkyl, and each R⁶ is independently C₃-C₇ cycloalkyl;
R⁷ is C₁-C₆ alkyl, and each R⁶ is independently halo;
R⁷ is C₁-C₆ alkyl and each R⁶ is independently halo;
R⁷ is C₁-C₆ alkyl, and R⁶ is cyano;
R⁷ is C₃-C₇ cycloalkyl, and each R⁶ is independently C₃-C₇ cycloalkyl;
R⁷ is C₃-C₇ cycloalkyl, and each R⁶ is independently halo;
R⁷ is C₃-C₇ cycloalkyl and each R⁶ is independently halo;
R⁷ is C₁-C₆ alkyl, and each R⁶ is independently C₁-C₆ alkoxy optionally substituted with one or more halo;
R⁷ is C₁-C₆ alkyl, and each R⁶ is independently C₁-C₆ alkoxy;
R⁷ is C₁-C₆ alkyl, and each R⁶ is independently C₁-C₆ alkoxy substituted with one or more halo;
R⁷ is halo, and each R⁶ is independently C₁-C₆ haloalkyl;
R⁷ is halo, and each R⁶ is independently C₁-C₆ haloalkoxy;
R⁷ is C₁-C₆ alkoxy; and each R⁶ is independently halo;
R⁷ is C₁-C₆ alkoxy; and R⁶ is chloro;
two pairs, each of one R⁶ and one R⁷, are on adjacent atoms, and each pair of one R⁶ and one R⁷ taken together with the atoms connecting them form a C₄-C₈ aliphatic carbocyclic ring;
and one R⁷ is halo and each pair of one R⁶ and one R⁷ taken together with the atoms connecting them form a C₄-C₆ aliphatic carbocyclic ring optionally substituted with one or more hydroxy, oxo, or C₁-C₆ alkyl; and one R⁷ is halo or cyano; or
two pairs, each of one R⁶ and one R⁷, are on adjacent atoms, and each pair of one R⁶ and one R⁷ taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl; and one R⁷ is halo or cyano.

In some embodiments, of the compound of formula AA,

the substituted ring B is

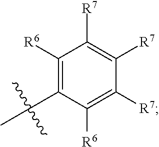

and R⁶ and R⁷ are one of the following combinations:
each R⁶ is isopropyl; and each R⁷ is methyl;
each R⁶ is isopropyl; and each R⁷ is isopropyl;
each R⁶ is isopropyl; and each R⁷ is trifluoromethyl;
each R⁶ is isopropyl; and each R⁷ is cyclopropyl;
each R⁶ is isopropyl; and each R⁷ is chloro;
each R⁶ is isopropyl; and each R⁷ is fluoro;
each R⁶ is ethyl; and each R⁷ is fluoro;
each R⁶ is isopropyl; and each R⁷ is cyano;
each R⁶ is cyclopropyl; and each R⁷ is cyclopropyl;
each R⁶ is cyclopropyl; and each R⁷ is chloro;
each R⁶ is cyclopropyl; and each R⁷ is fluoro;
each R⁶ is isopropyl; and each R⁷ is methoxy;
each R⁶ is isopropyl; and each R⁷ is trifluoromethoxy;
each R⁶ is chloro; and each R⁷ is trifluoromethyl;
each R⁶ is chloro; and each R⁷ is trifluoromethoxy;
each R⁷ is isopropyl; and each R⁶ is methyl;
each R⁷ is isopropyl; and each R⁶ is trifluoromethyl;
each R⁷ is isopropyl; and each R⁶ is cyclopropyl;
each R⁷ is isopropyl; and each R⁶ is chloro;
each R⁷ is ethyl; and each R⁶ is fluoro;
each R⁷ is isopropyl; and each R⁶ is cyano;
each R⁷ is cyclopropyl; and each R⁶ is cyclopropyl;
each R⁷ is cyclopropyl; and each R⁶ is chloro;
each R⁷ is cyclopropyl; and each R⁶ is fluoro;
each R⁷ is isopropyl; and each R⁶ is methoxy;
each R⁷ is isopropyl; and each R⁶ is trifluoromethoxy;
each R⁷ is chloro; and each R⁶ is trifluoromethyl;
each R⁷ is chloro; and each R⁶ is trifluoromethoxy;
each R⁶ is isopropyl; two R⁷ are fluoro; and one R⁷ is chloro;
two pairs, each of one R⁶ and one R⁷, are on adjacent atoms, and each pair of one R⁶ and one R⁷ taken together with the atoms connecting them form a C₅ aliphatic carbocyclic ring; and one R⁷ is chloro;
(i) two pairs, each of one R⁶ and one R⁷, are on adjacent atoms, and each pair of one R⁶ and one R⁷ taken together with the atoms connecting them form a C₄ aliphatic carbocyclic ring optionally substituted with one or more hydroxy, oxo, or methyl; and one R⁷ is fluoro or chloro;
(ii) two pairs, each of one R⁶ and one R⁷, are on adjacent atoms, and each pair of one R⁶ and one R⁷ taken together with the atoms connecting them form a C₅ aliphatic carbocyclic ring optionally substituted with one or more hydroxy, oxo, or methyl; and one R⁷ is fluoro or chloro;
(iii) two pairs, each of one R⁶ and one R⁷, are on adjacent atoms, and each pair of one R⁶ and one R⁷ taken together with the atoms connecting them form a C₆ aliphatic carbocyclic ring optionally substituted with one or more hydroxy, oxo, or methyl; and one R⁷ is fluoro or chloro;
(iv) two pairs, each of one R⁶ and one R⁷, are on adjacent atoms, and each pair of one R⁶ and one R⁷ taken together with the atoms connecting them form a 5-membered heterocyclic ring containing 1 heteroatom independently selected from O, N, and S, wherein the heterocyclic ring is optionally substituted with one or more hydroxy, oxo, or methyl; and one R⁷ is fluoro or chloro or
two pairs, each of one R⁶ and one R⁷, are on adjacent atoms, and each pair of one R⁶ and one R⁷ taken together with the atoms connecting them form a 6-membered heterocyclic ring containing 1 heteroatom independently selected from O, N, and S, wherein the heterocyclic ring is optionally substituted with one or more hydroxy, oxo, or methyl; and one R⁷ is fluoro or chloro.

[A]

In some embodiments, the optionally substituted ring A is

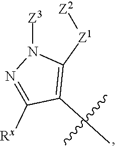

wherein R^{x} is selected from the group consisting of H and C₁-C₆ alkyl (e.g., methyl); Z¹ is selected from the group consisting of O, NH, and —CH₂— optionally substituted with 1-2 R²⁰; Z² is selected from the group consisting of NH and —CH₂— optionally substituted with 1-2 R²⁰; Z³ is selected from the group consisting of —CH₂— optionally substituted with 1-2 R²⁰, —CH₂CH₂— optionally substituted with 1-2 R²⁰, and —CH₂CH₂CH₂— optionally substituted with 1-2 R²⁰; R²⁰ is selected from the group consisting of hydroxy, halo (e.g., fluoro), oxo, C₁-C₆ alkyl (e.g., methyl or ethyl) optionally substituted with one R²¹, C₁-C₆ alkoxy (e.g., methoxy, ethoxy, or isopropoxy) optionally substituted with one R²¹, NR⁸R⁹, 3- to 10-membered heterocycloalkyl (e.g., azetidinyl or pyrrolidinyl) optionally substituted with one R²¹, or one pair of R²⁰ on the same atom, taken together with the atom connecting them, independently forms a monocyclic C₃-C₄ carbocyclic ring or a monocyclic 3- to 4-membered heterocyclic ring containing 1 O atom optionally substituted with OS(O)₂Ph; R²¹ is selected from the group consisting of halo (e.g., fluoro), NR⁸R⁹, C₂-C₆ alkynyl (e.g., ethynyl), and C₁-C₆ alkoxy (e.g., methoxy); R⁸ and R⁹ at each occurrence is independently selected from hydrogen, C₁-C₆ alkyl (e.g., methyl or ethyl), COR¹³, and CO₂R¹³; R¹³ is selected from the group consisting of: C₁-C₆ alkyl (e.g., methyl or t-butyl) and C₁-C₆ haloalkyl (e.g., trifluoromethyl); and

the substituted ring B is selected from the group consisting of:

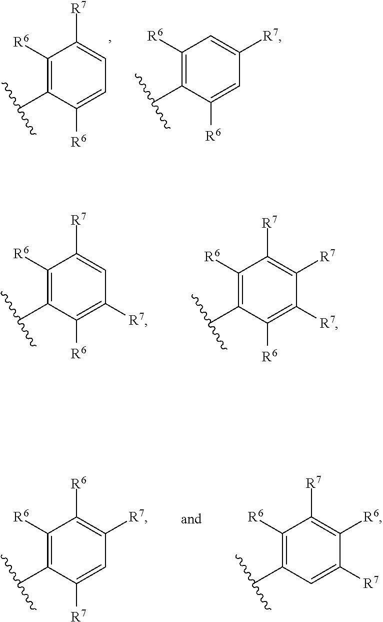

wherein
each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;

or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

[B]

In some embodiments, the optionally substituted ring A is

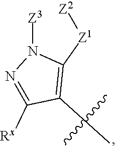

wherein R^{x} is selected from the group consisting of H and C₁-C₆ alkyl (e.g., methyl); Z¹ is selected from the group consisting of O, NH, and —CH₂— optionally substituted with 1-2 R²⁰; Z² is selected from the group consisting of NH and —CH₂— optionally substituted with 1-2 R²⁰; Z³ is selected from the group consisting of —CH₂— optionally substituted with 1-2 R²⁰, —CH₂CH₂— optionally substituted with 1-2 R²⁰, and —CH₂CH₂CH₂— optionally substituted with 1-2 R²⁰; R²⁰ is selected from the group consisting of hydroxy, halo (e.g., fluoro), oxo, C₁-C₆ alkyl (e.g., methyl or ethyl) optionally substituted with one R²¹, C₁-C₆ alkoxy (e.g., methoxy, ethoxy, or isopropoxy) optionally substituted with one R²¹, NR⁸R⁹, 3- to 10-membered heterocycloalkyl (e.g., azetidinyl or pyrrolidinyl) optionally substituted with one R²¹, or one pair of R²⁰ on the same atom, taken together with the atom connecting them, independently forms a monocyclic C₃-C₄ carbocyclic ring or a monocyclic 3- to 4-membered heterocyclic ring containing 1 O atom optionally substituted with OS(O)₂Ph; R²¹ is selected from the group consisting of halo (e.g., fluoro), NR⁸R⁹, C₂-C₆ alkynyl (e.g., ethynyl), and C₁-C₆ alkoxy (e.g., methoxy); R⁸ and R⁹ at each occurrence is independently selected from hydrogen, C₁-C₆ alkyl (e.g., methyl or ethyl), COR¹³, and CO₂R¹³; R¹³ is selected from the group consisting of: C₁-C₆ alkyl (e.g., methyl or t-butyl) and C₁-C₆ haloalkyl (e.g., trifluoromethyl); and

the substituted ring B is selected from:

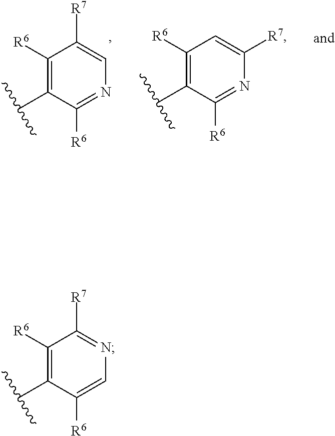

wherein
each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein the C₆-C₁₀ aryl is optionally substituted with one to two C₁-C₆ alkyl optionally substituted with one to three halo; and wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;

or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

[C]

In some embodiments, The compound of any one of claims 1-2, wherein

the optionally substituted ring A is

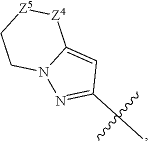

wherein Z⁴ is selected from the group consisting of —CH₂—, —C(O)—, and NH; Z⁵ is selected from the group consisting of O, NH, N—CH₃, and —CH₂—.

the substituted ring B is selected from:

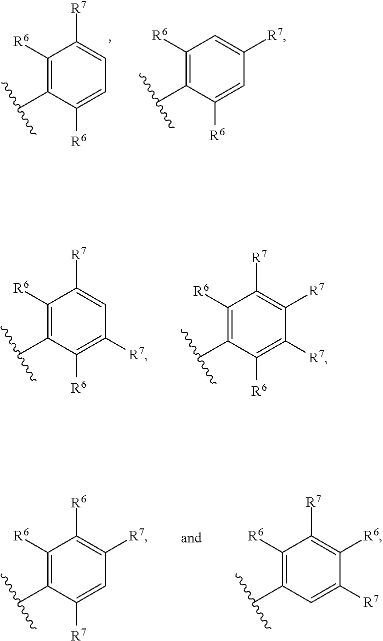

wherein

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;

or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

[D]

In some embodiments, the optionally substituted ring A is

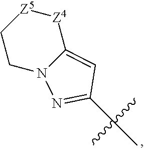

wherein Z⁴ is selected from the group consisting of —CH₂—, —C(O)—, and NH; Z⁵ is selected from the group consisting of O, NH, N—CH₃, and —CH₂—.

the substituted ring B is selected from:

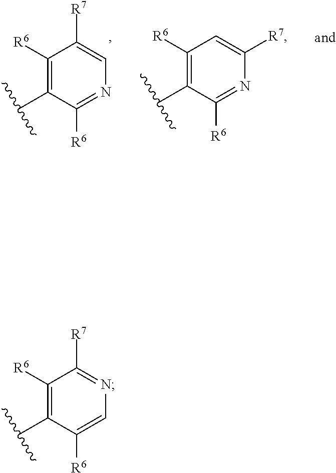

wherein

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein the C₆-C₁₀ aryl is optionally substituted with one to two C₁-C₆ alkyl optionally substituted with one to three halo; and wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;

or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments of [A] and [C], the substituted ring B is selected from:

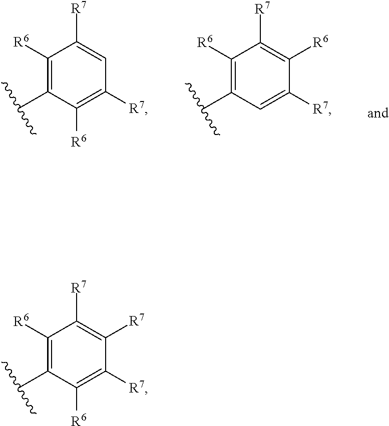

wherein each pair of R⁶ and R⁷ on adjacent atoms taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR10, COOC₁-C₆ alkyl, C₆-C₁₀ aryl, and CONR⁸R⁹.

In certain of the foregoing embodiments, the remaining R⁷ if present is independently cyano or halo (e.g., halo (e.g., F)).

In some embodiments of [A] and [C], the substituted ring B is selected from:

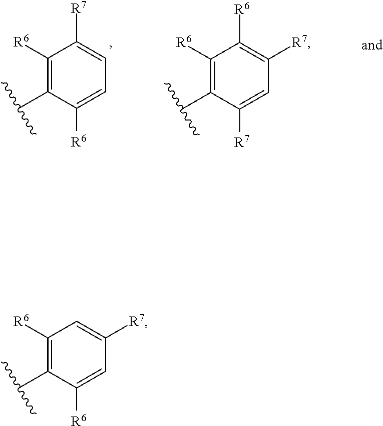

wherein

each R⁶ and R⁷ is independently C₁-C₆ alkyl, C₁-C₆ haloalkyl, halo, —CN, C₃-C₇ cycloalkyl.

In some embodiments of [A] and [C], the substituted ring B is:

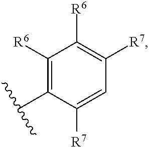

wherein

one pair of R⁶ and R⁷ on adjacent atoms taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹;

each of the remaining R⁶ and R⁷ is independently C₁-C₆ alkyl.

In one embodiment, provided herein is a combination of a compound of any preceding embodiment, for use in the treatment or the prevention of a condition mediated by TNF-α, in a patient in need thereof, wherein the compound is administered to said patient at a therapeutically effective amount. Preferably, the subject is resistant to treatment with an anti-TNFα agent. Preferably, the condition is a gut disease or disorder.

In one embodiment, provided herein is a pharmaceutical composition of comprising a compound of any preceding embodiment, and an anti-TNFα agent disclosed herein. Preferably wherein the anti-TNFα agent is Infliximab, Etanercept, Certolizumab pegol, Golimumab or Adalimumab, more preferably wherein the anti-TNFα agent is Adalimumab.

In one embodiment, provided herein is a pharmaceutical combination of a compound of any preceding embodiment, and an anti-TNFα agent Preferably wherein the anti-TNFα agent is Infliximab, Etanercept, Certolizumab pegol, Golimumab or Adalimumab, more preferably wherein the anti-TNFα agent is Adalimumab.

In one embodiment, the present invention relates to an NLRP3 antagonist for use in the treatment or the prevention of a condition mediated by TNF-α, in particular a gut disease or disorder, in a patient in need thereof, wherein the NLRP3 antagonist is administered to said patient at a therapeutically effective amount.

In one embodiment, the present invention relates to an NLRP3 antagonist for use in the treatment or the prevention of a condition, in particular a gut disease or disorder, in a patient in need thereof wherein the NLRP3 antagonist is administered to said patient at a therapeutically effective amount.

In one embodiment, the present invention relates to an NLRP3 antagonist for use in the treatment, stabilization or lessening the severity or progression of gut disease or disorder, in a patient in need thereof wherein the NLRP3 antagonist is administered to said patient at a therapeutically effective amount.

In one embodiment, the present invention relates to an NLRP3 antagonist for use in the slowing, arresting, or reducing the development of a gut disease or disorder, in a patient in need thereof wherein the NLRP3 antagonist is administered to said patient at a therapeutically effective amount.

In one embodiment, the present invention relates to an NLRP3 antagonist for use according to above listed embodiments wherein the NLRP3 antagonist is a gut-targeted NLRP3 antagonist.

In one embodiment, the present invention relates ton NLRP3 antagonist for use according to any of the above embodiments, wherein the gut disease is IBD.

In one embodiment, the present invention relates to an NLRP3 antagonist for use according to any of the above embodiments, wherein the gut disease is US or CD.

In one embodiment, the present invention relates to a method for the treatment or the prevention of a condition mediated by TNF-α, in particular a gut disease or disorder, in a patient in need thereof, comprising administering to said patient a therapeutically effective amount of a gut-targeted NLRP3 antagonist.

In one embodiment, the present invention relates to a method for the treatment or the prevention of a condition, in particular a gut disease or disorder, in a patient in need thereof, comprising administering to said patient a therapeutically effective amount of a gut-targeted NLRP3 antagonist.

In one embodiment, the present invention relates to a method for the treatment, stabilization or lessening the severity or progression of gut disease or disorder, in a patient in need thereof comprising administering to said patient a therapeutically effective amount of a gut-targeted NLRP3 antagonist.

In one embodiment, the present invention relates to a method for slowing, arresting, or reducing the development of a gut disease or disorder, in a patient in need thereof comprising administering to said patient a therapeutically effective amount of a gut-targeted NLRP3 antagonist.

In one embodiment, the present invention relates to a method according to any of the above embodiments, wherein the gut disease is IBD.

In one embodiment, the present invention relates to a method according to any of the above embodiments x to xx, wherein the gut disease is UC or CD.

In one embodiment, the present invention relates to a method for the treatment or the prevention of a condition mediated by TNF-α, in particular a gut disease or disorder, in a patient in need thereof, comprising administering to said patient a therapeutically effective amount of a gut-targeted NLRP3 antagonist.

Unless otherwise indicated, when a disclosed compound is named or depicted by a structure without specifying the stereochemistry and has one or more chiral centers, it is understood to represent all possible stereoisomers of the compound.

It is understood that the combination of variables in the formulae herein is such that the compounds are stable.

In some embodiments, provided herein is a compound that is selected from the group consisting of the compounds in Table 1:

TABLE 1

| Cmpd | | Cmpd | |
|---|---|---|---|
| # | Structure | # | Structure |
| 101 | 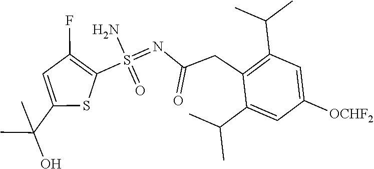 [SMILES: CC(C)c1cc(OC(F)F)cc(C(C)C)c1CC(=O)N=S(N)(=O)c1sc(C(C)(C)O)cc1F] | 140b | 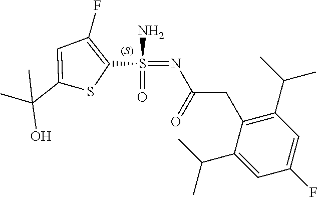 |
| 101a | 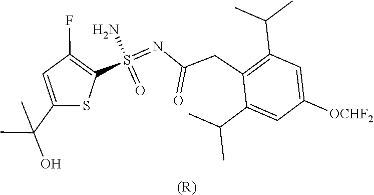 [SMILES: CC(C)c1cc(OC(F)F)cc(C(C)C)c1CC(=O)N=[S@@](N)(=O)c1sc(C(C)(C)O)cc1F ; "(R)"] | 141 | 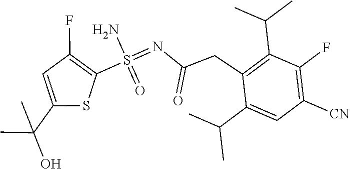 [SMILES: CC(C)c1cc(C#N)c(F)c(C(C)C)c1CC(=O)N=S(N)(=O)c1sc(C(C)(C)O)cc1F] |
| 101b | 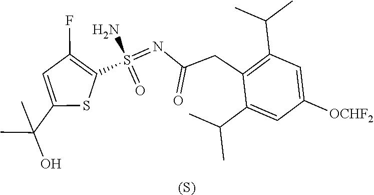 [SMILES: CC(C)c1cc(OC(F)F)cc(C(C)C)c1CC(=O)N=[S@](N)(=O)c1sc(C(C)(C)O)cc1F ; "(S)"] | 141a | 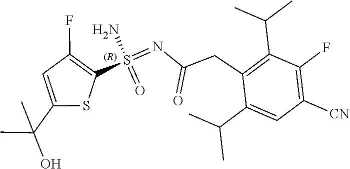 |
| 102 | 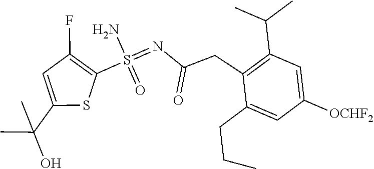 [SMILES: CCCc1cc(OC(F)F)cc(C(C)C)c1CC(=O)N=S(N)(=O)c1sc(C(C)(C)O)cc1F] | 141b | 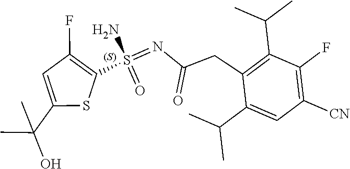 |
| 103 | 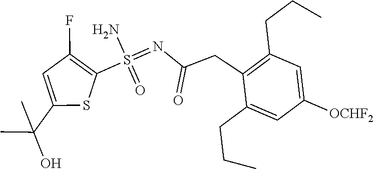 [SMILES: CCCc1cc(OC(F)F)cc(CCC)c1CC(=O)N=S(N)(=O)c1sc(C(C)(C)O)cc1F] | 142 | 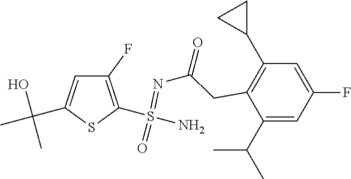 [SMILES: CC(C)c1cc(F)cc(C2CC2)c1CC(=O)N=S(N)(=O)c1sc(C(C)(C)O)cc1F] |
| 104 | 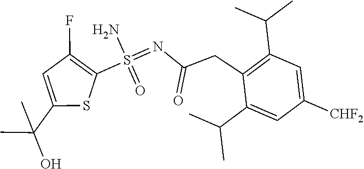 [SMILES: CC(C)c1cc(C(F)F)cc(C(C)C)c1CC(=O)N=S(N)(=O)c1sc(C(C)(C)O)cc1F] | 143 | 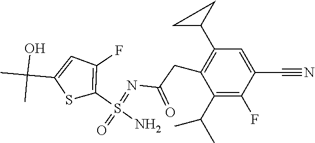 [SMILES: CC(C)c1c(F)c(C#N)cc(C2CC2)c1CC(=O)N=S(N)(=O)c1sc(C(C)(C)O)cc1F] |
| 104a | 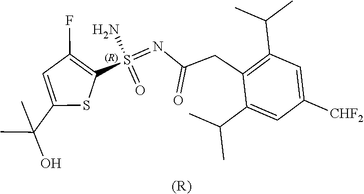 | 144 | 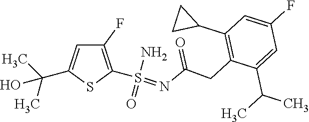 [SMILES: CC(C)c1cc(F)cc(C2CC2)c1CC(=O)N=S(N)(=O)c1sc(C(C)(C)O)cc1F] |
| 104b | 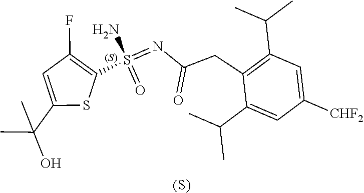 | 144a | 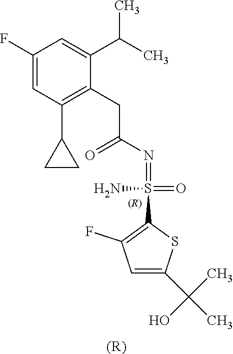 |
| 105 | 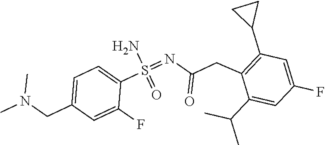 [SMILES: CC(C)c1cc(F)cc(C2CC2)c1CC(=O)N=S(N)(=O)c1ccc(CN(C)C)cc1F] | 144b | 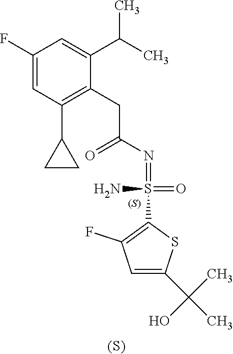 |
| 106 | 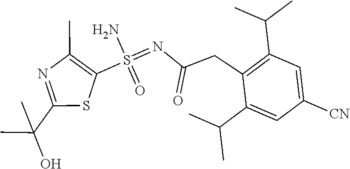 [SMILES: Cc1nc(C(C)(C)O)sc1S(N)(=O)=NC(=O)Cc1c(C(C)C)cc(C#N)cc1C(C)C] | 145 | |
| 106a | 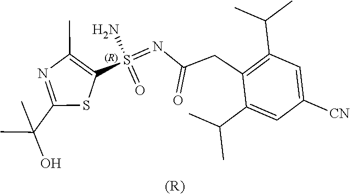 | 145a | (R) |
| 106b | 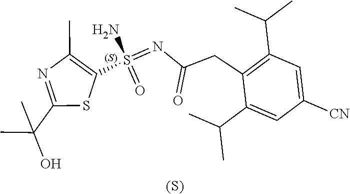 | | (S) |
| 107 | 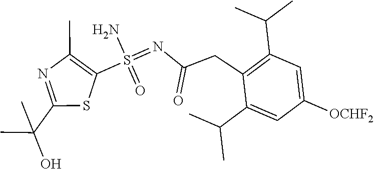 [SMILES: Cc1nc(C(C)(C)O)sc1S(N)(=O)=NC(=O)Cc1c(C(C)C)cc(OC(F)F)cc1C(C)C] | 146 | |
| 107a | 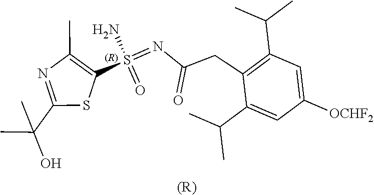 | 147 | 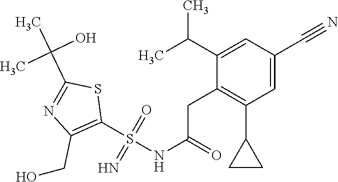 [SMILES: CC(C)c1cc(C#N)cc(C2CC2)c1CC(=O)NS(=N)(=O)c1sc(C(C)(C)O)nc1CO] |
| 107b | 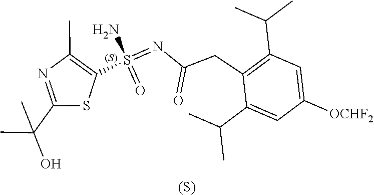 | 147a | 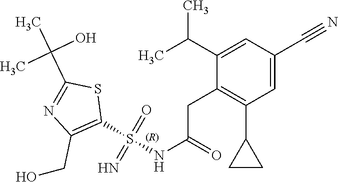 |
| 108 | 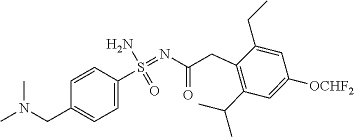 [SMILES: CCc1cc(OC(F)F)cc(C(C)C)c1CC(=O)N=S(N)(=O)c1ccc(CN(C)C)cc1] | 147b | 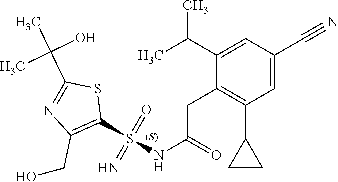 |
| 109 | 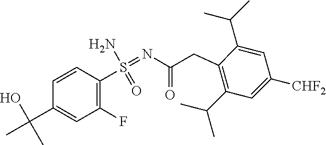 [SMILES: CC(C)c1cc(C(F)F)cc(C(C)C)c1CC(=O)N=S(N)(=O)c1ccc(C(C)(C)O)cc1F] | 148 | 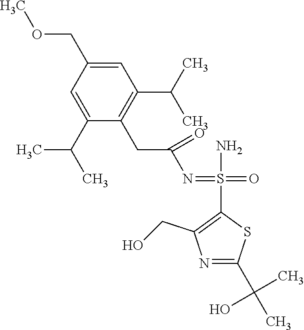 [SMILES: COCc1cc(C(C)C)c(CC(=O)N=S(N)(=O)c2sc(C(C)(C)O)nc2CO)c(C(C)C)c1] |
| 110 | 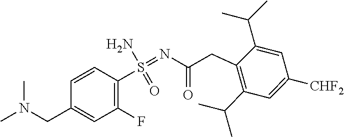 [SMILES: CC(C)c1cc(C(F)F)cc(C(C)C)c1CC(=O)N=S(N)(=O)c1ccc(CN(C)C)cc1F] | 148a | |
| 110a′ | | 148b | |
| 110b′ | | 149a | |
| 110a | 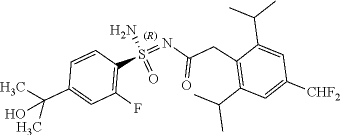 | 149b | |
| 110b | 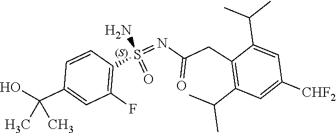 | 150 | 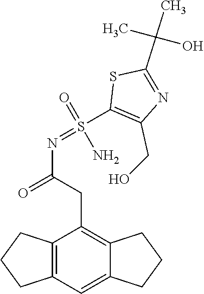 [SMILES: CC(C)(O)c1nc(CO)c(S(N)(=O)=NC(=O)Cc2c3c(cc4c2CCC4)CCC3)s1] |
| 111 | 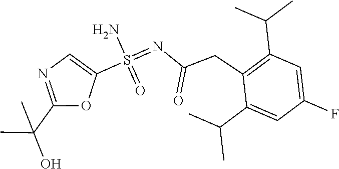 [SMILES: CC(C)c1cc(F)cc(C(C)C)c1CC(=O)N=S(N)(=O)c1cnc(C(C)(C)O)o1] | 150a | 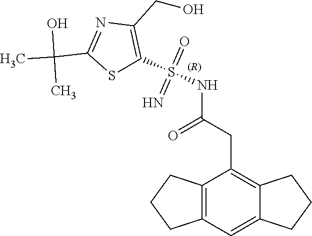 |
| 112 | 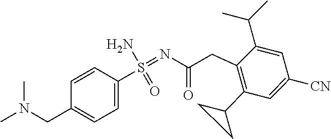 [SMILES: CC(C)c1cc(C#N)cc(C2CC2)c1CC(=O)N=S(N)(=O)c1ccc(CN(C)C)cc1] | 150b | 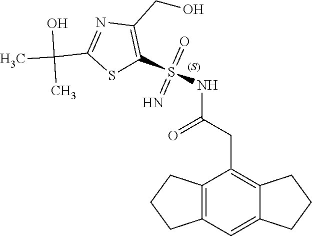 |
| 112a | 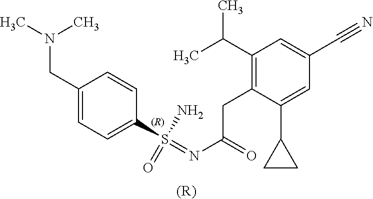 | 151 | |
| 112b | 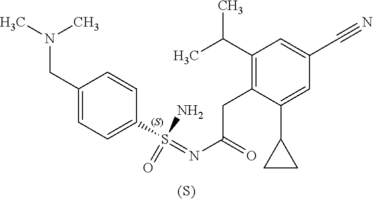 | 152 | 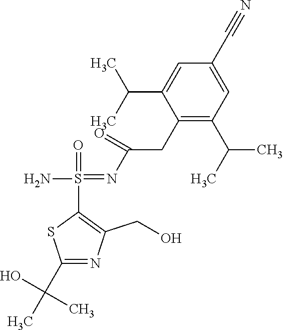 [SMILES: CC(C)c1cc(C#N)cc(C(C)C)c1CC(=O)N=S(N)(=O)c1sc(C(C)(C)O)nc1CO] |
| 113 | 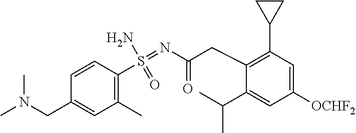 [SMILES: Cc1cc(CN(C)C)ccc1S(N)(=O)=NC(=O)Cc1c(C(C)C)cc(OC(F)F)cc1C1CC1] | 152a | |
| 114 | 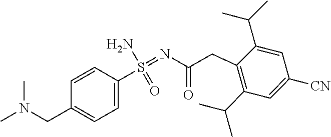 [SMILES: CC(C)c1cc(C#N)cc(C(C)C)c1CC(=O)N=S(N)(=O)c1ccc(CN(C)C)cc1] | 152b | |
| 114a | 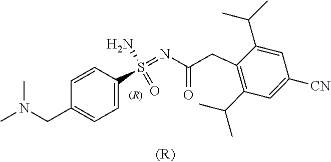 | 153 | 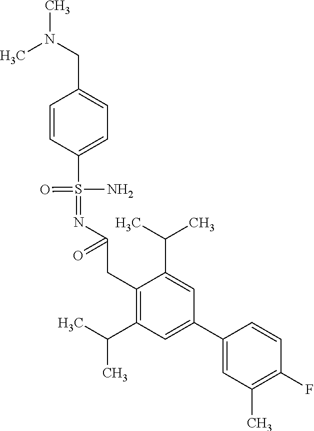 [SMILES: Cc1cc(-c2cc(C(C)C)c(CC(=O)N=S(N)(=O)c3ccc(CN(C)C)cc3)c(C(C)C)c2)ccc1F] |
| 114b | 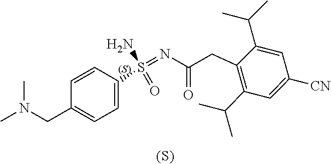 | 154 | 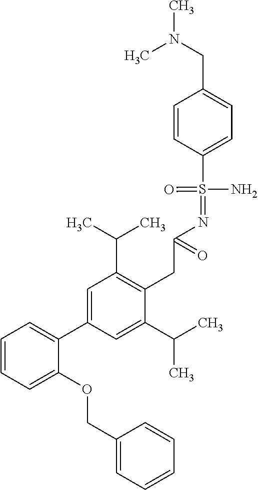 [SMILES: CC(C)c1cc(-c2ccccc2OCc2ccccc2)cc(C(C)C)c1CC(=O)N=S(N)(=O)c1ccc(CN(C)C)cc1] |
| 115 | 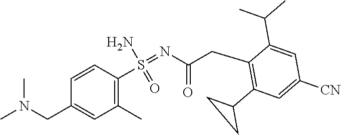 [SMILES: Cc1cc(CN(C)C)ccc1S(N)(=O)=NC(=O)Cc1c(C(C)C)cc(C#N)cc1C1CC1] | 155 | 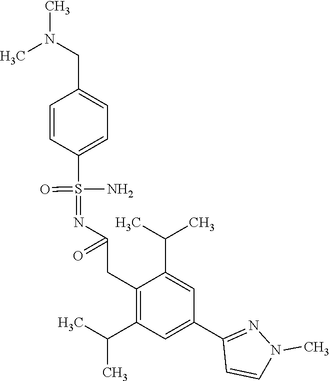 |
| 116 | 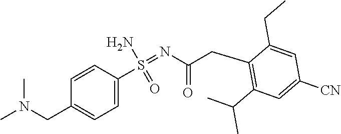 [SMILES: CCc1cc(C#N)cc(C(C)C)c1CC(=O)N=S(N)(=O)c1ccc(CN(C)C)cc1] | 156 | 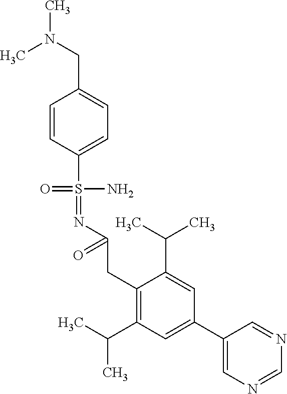 [SMILES: CC(C)c1cc(-c2cncnc2)cc(C(C)C)c1CC(=O)N=S(N)(=O)c1ccc(CN(C)C)cc1] |
| 116a | 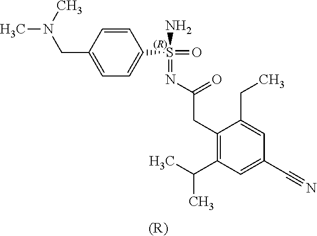 | 157 | 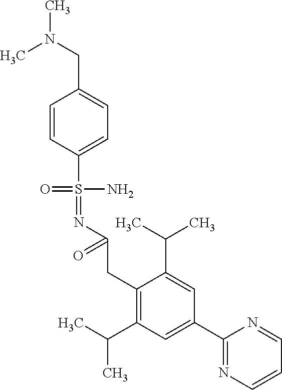 [SMILES: CC(C)c1cc(-c2ncccn2)cc(C(C)C)c1CC(=O)N=S(N)(=O)c1ccc(CN(C)C)cc1] |
| 116b | 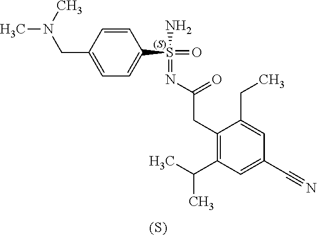 | 158 | 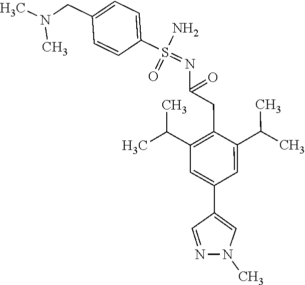 [SMILES: CC(C)c1cc(-c2cnn(C)c2)cc(C(C)C)c1CC(=O)N=S(N)(=O)c1ccc(CN(C)C)cc1] |
| 117 | 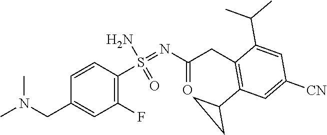 [SMILES: CC(C)c1cc(C#N)cc(C2CC2)c1CC(=O)N=S(N)(=O)c1ccc(CN(C)C)cc1F] | 159 | 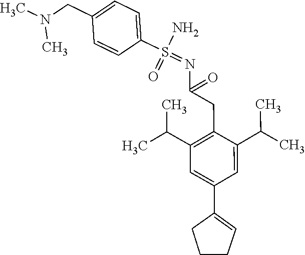 [SMILES: CC(C)c1cc(C2=CCCC2)cc(C(C)C)c1CC(=O)N=S(N)(=O)c1ccc(CN(C)C)cc1] |
| 117a | 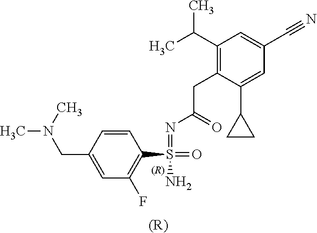 | 160 | 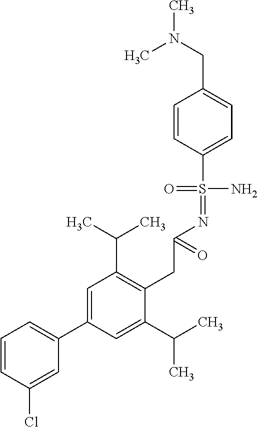 [SMILES: CC(C)c1cc(-c2cccc(Cl)c2)cc(C(C)C)c1CC(=O)N=S(N)(=O)c1ccc(CN(C)C)cc1] |
| 117b | 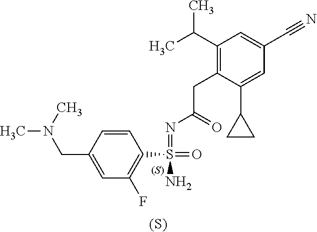 | 160 | 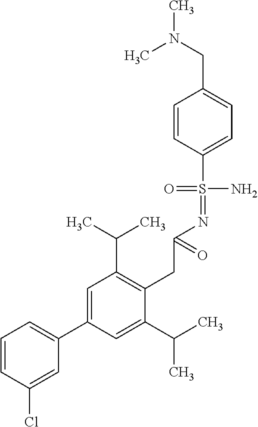 [SMILES: CC(C)c1cc(-c2cccc(Cl)c2)cc(C(C)C)c1CC(=O)N=S(N)(=O)c1ccc(CN(C)C)cc1] |
| 118 | 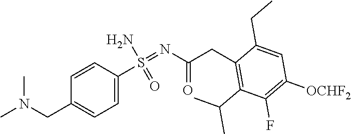 [SMILES: CCc1cc(OC(F)F)c(F)c(C(C)C)c1CC(=O)N=S(N)(=O)c1ccc(CN(C)C)cc1] | 162 | 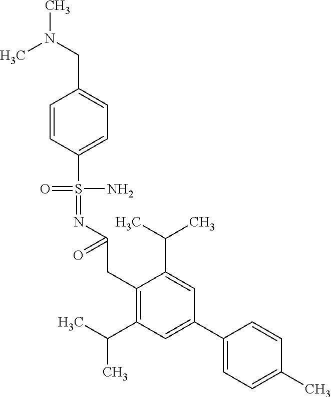 |
| 119 | 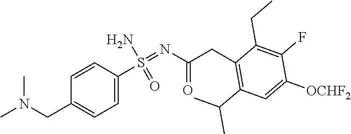 [SMILES: CCc1c(F)c(OC(F)F)cc(C(C)C)c1CC(=O)N=S(N)(=O)c1ccc(CN(C)C)cc1] | 163 | 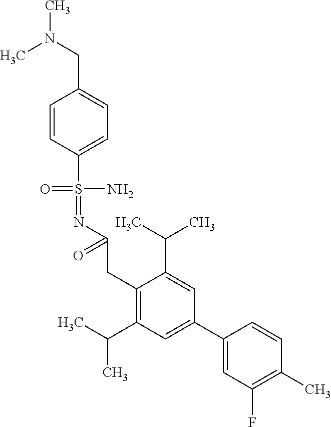 |
| 120 | 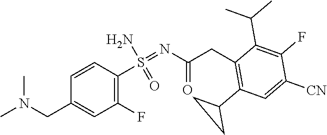 [SMILES: CC(C)c1c(F)c(C#N)cc(C2CC2)c1CC(=O)N=S(N)(=O)c1ccc(CN(C)C)cc1F] | 164 | 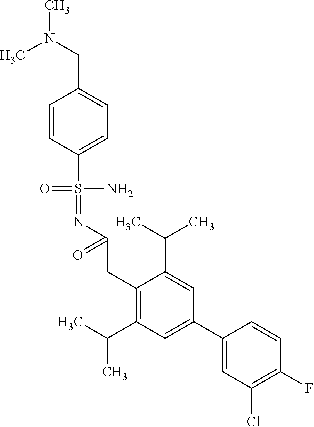 [SMILES: CC(C)c1cc(-c2ccc(F)c(Cl)c2)cc(C(C)C)c1CC(=O)N=S(N)(=O)c1ccc(CN(C)C)cc1] |
| 121 | 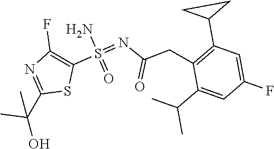 [SMILES: CC(C)c1cc(F)cc(C2CC2)c1CC(=O)N=S(N)(=O)c1sc(C(C)(C)O)nc1F] | 165 | 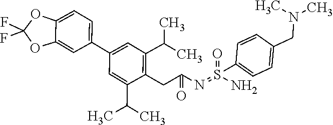 [SMILES: CC(C)c1cc(-c2ccc3c(c2)OC(F)(F)O3)cc(C(C)C)c1CC(=O)N=S(N)(=O)c1ccc(CN(C)C)cc1] |
| 122 | 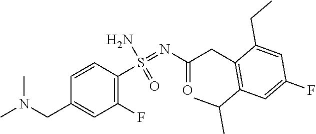 [SMILES: CCc1cc(F)cc(C(C)C)c1CC(=O)N=S(N)(=O)c1ccc(CN(C)C)cc1F] | 166 | 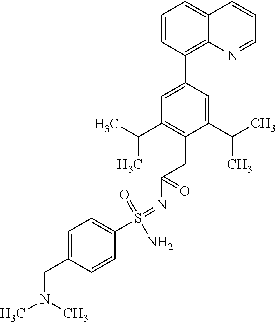 [SMILES: CC(C)c1cc(-c2cccc3cccnc23)cc(C(C)C)c1CC(=O)N=S(N)(=O)c1ccc(CN(C)C)cc1] |
| 123 | 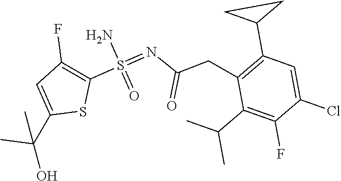 [SMILES: CC(C)c1c(F)c(Cl)cc(C2CC2)c1CC(=O)N=S(N)(=O)c1sc(C(C)(C)O)cc1F] | 167 | 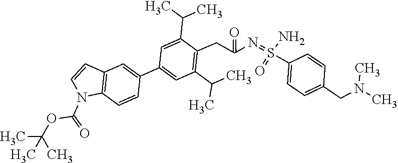 [SMILES: CC(C)c1cc(-c2ccc3c(ccn3C(=O)OC(C)(C)C)c2)cc(C(C)C)c1CC(=O)N=S(N)(=O)c1ccc(CN(C)C)cc1] |
| 123a | 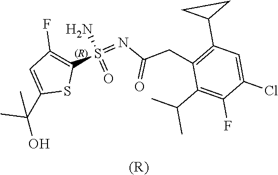 | 168 | 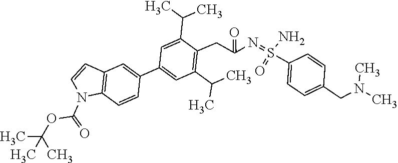 [SMILES: CC(C)c1cc(-c2ccc3c(ccn3C(=O)OC(C)(C)C)c2)cc(C(C)C)c1CC(=O)N=S(N)(=O)c1ccc(CN(C)C)cc1] |
| 123b | 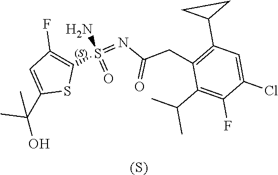 | 169 | 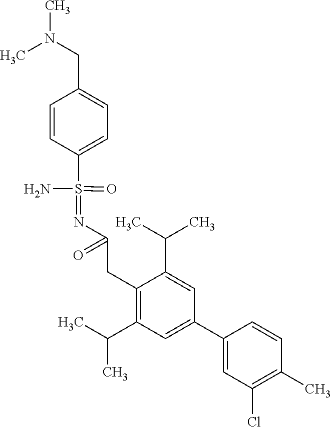 |
| 124 | 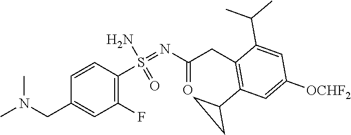 [SMILES: CC(C)c1cc(OC(F)F)cc(C2CC2)c1CC(=O)N=S(N)(=O)c1ccc(CN(C)C)cc1F] | 170 | 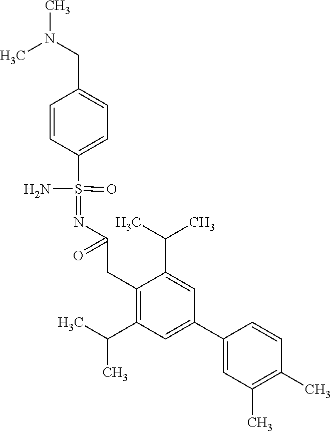 |
| 125 | 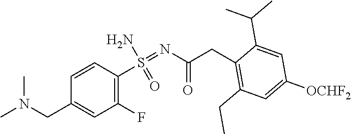 [SMILES: CCc1cc(OC(F)F)cc(C(C)C)c1CC(=O)N=S(N)(=O)c1ccc(CN(C)C)cc1F] | 171 | 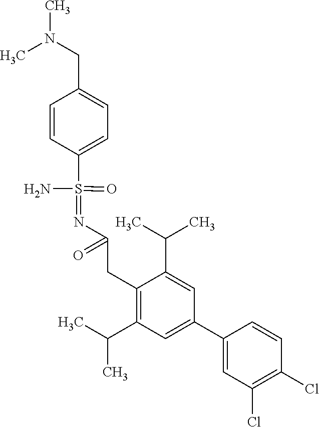 [SMILES: CC(C)c1cc(-c2ccc(Cl)c(Cl)c2)cc(C(C)C)c1CC(=O)N=S(N)(=O)c1ccc(CN(C)C)cc1] |
| 126 | 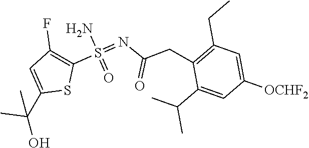 [SMILES: CCc1cc(OC(F)F)cc(C(C)C)c1CC(=O)N=S(N)(=O)c1sc(C(C)(C)O)cc1F] | 172 | 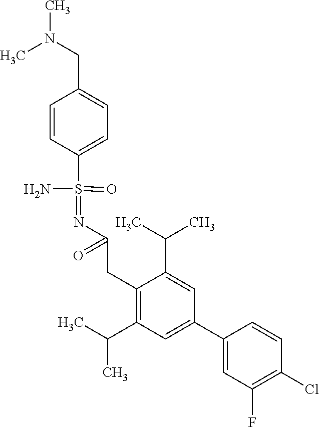 [SMILES: CC(C)c1cc(-c2ccc(Cl)c(F)c2)cc(C(C)C)c1CC(=O)N=S(N)(=O)c1ccc(CN(C)C)cc1] |
| 126a | 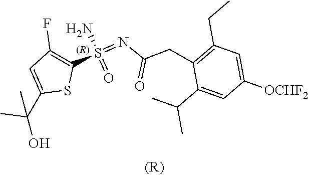 | 173 | 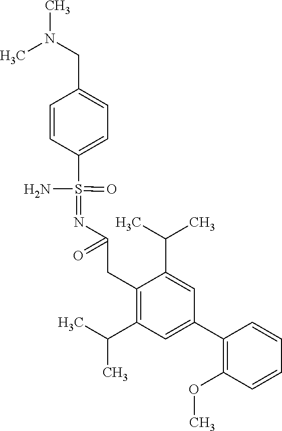 [SMILES: COc1ccccc1-c1cc(C(C)C)c(CC(=O)N=S(N)(=O)c2ccc(CN(C)C)cc2)c(C(C)C)c1] |
| 126b | 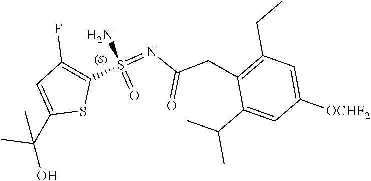 | 174 | 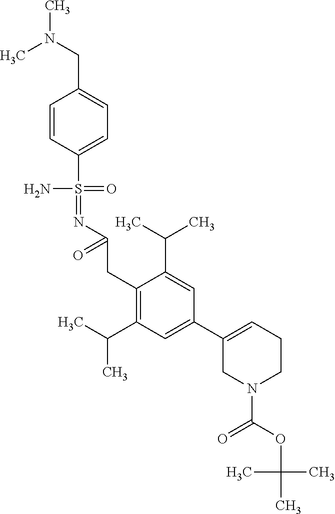 [SMILES: CC(C)c1cc(C2=CCCN(C(=O)OC(C)(C)C)C2)cc(C(C)C)c1CC(=O)N=S(N)(=O)c1ccc(CN(C)C)cc1] |
| 127 | 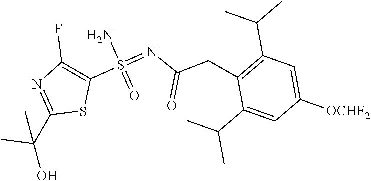 [SMILES: CC(C)c1cc(OC(F)F)cc(C(C)C)c1CC(=O)N=S(N)(=O)c1sc(C(C)(C)O)nc1F] | 175 | 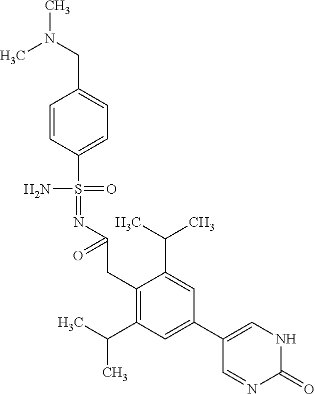 [SMILES: CC(C)c1cc(-c2cnc(=O)[nH]c2)cc(C(C)C)c1CC(=O)N=S(N)(=O)c1ccc(CN(C)C)cc1] |
| 128 | 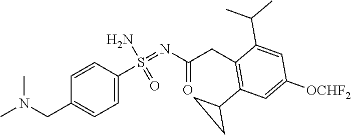 [SMILES: CC(C)c1cc(OC(F)F)cc(C2CC2)c1CC(=O)N=S(N)(=O)c1ccc(CN(C)C)cc1] | 176 | 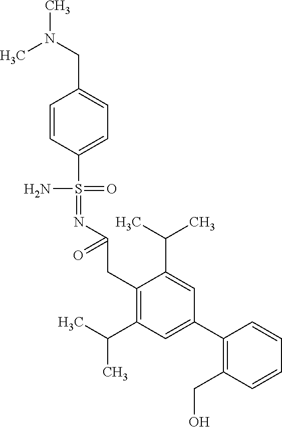 [SMILES: CC(C)c1cc(-c2ccccc2CO)cc(C(C)C)c1CC(=O)N=S(N)(=O)c1ccc(CN(C)C)cc1] |
| 129 | 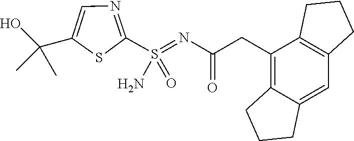 [SMILES: CC(C)(O)c1cnc(S(N)(=O)=NC(=O)Cc2c3c(cc4c2CCC4)CCC3)s1] | 177 | 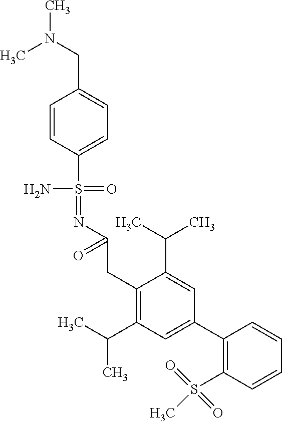 [SMILES: CC(C)c1cc(-c2ccccc2S(C)(=O)=O)cc(C(C)C)c1CC(=O)N=S(N)(=O)c1ccc(CN(C)C)cc1] |
| 129a | 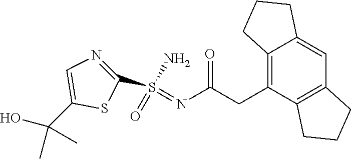 [SMILES: CC(C)(O)c1cnc([S@](N)(=O)=NC(=O)Cc2c3c(cc4c2CCC4)CCC3)s1] | 178 | 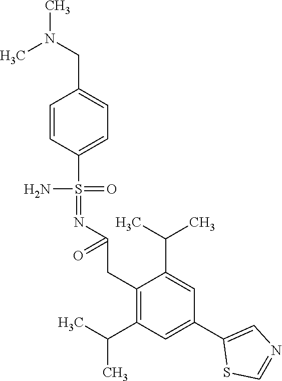 [SMILES: CC(C)c1cc(-c2cncs2)cc(C(C)C)c1CC(=O)N=S(N)(=O)c1ccc(CN(C)C)cc1] |
| 129b | 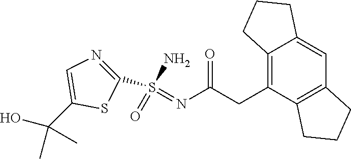 [SMILES: CC(C)(O)c1cnc([S@@](N)(=O)=NC(=O)Cc2c3c(cc4c2CCC4)CCC3)s1] | 179 | 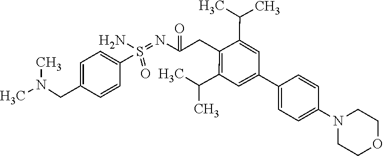 [SMILES: CC(C)c1cc(-c2ccc(N3CCOCC3)cc2)cc(C(C)C)c1CC(=O)N=S(N)(=O)c1ccc(CN(C)C)cc1] |
| 130 | 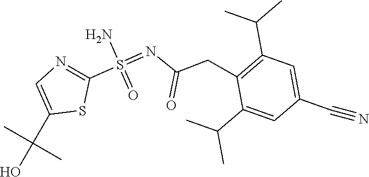 [SMILES: CC(C)c1cc(C#N)cc(C(C)C)c1CC(=O)N=S(N)(=O)c1ncc(C(C)(C)O)s1] | 180 | 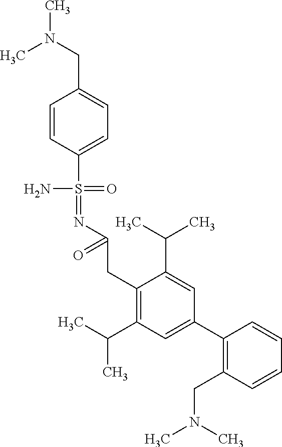 [SMILES: CC(C)c1cc(-c2ccccc2CN(C)C)cc(C(C)C)c1CC(=O)N=S(N)(=O)c1ccc(CN(C)C)cc1] |
| 130a | 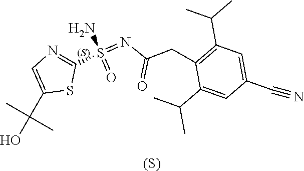 | 181 | 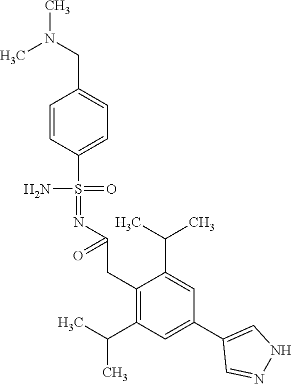 [SMILES: CC(C)c1cc(-c2cn[nH]c2)cc(C(C)C)c1CC(=O)N=S(N)(=O)c1ccc(CN(C)C)cc1] |
| 130b | 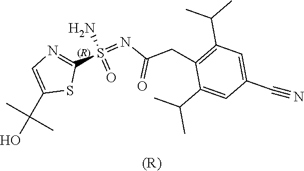 | 182 | 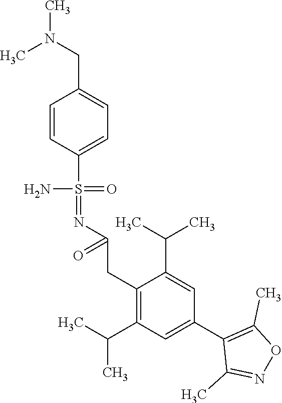 [SMILES: Cc1noc(C)c1-c1cc(C(C)C)c(CC(=O)N=S(N)(=O)c2ccc(CN(C)C)cc2)c(C(C)C)c1] |
| 131 | 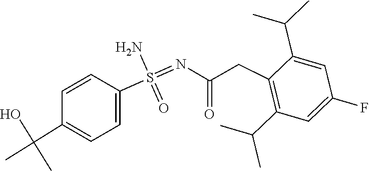 [SMILES: CC(C)c1cc(F)cc(C(C)C)c1CC(=O)N=S(N)(=O)c1ccc(C(C)(C)O)cc1] | 183 | 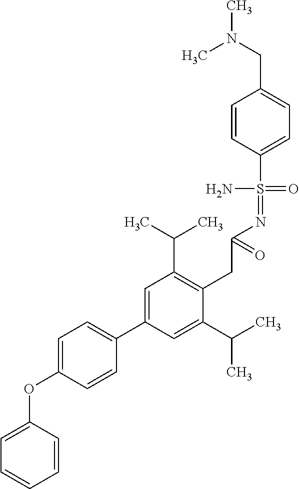 [SMILES: CC(C)c1cc(-c2ccc(Oc3ccccc3)cc2)cc(C(C)C)c1CC(=O)N=S(N)(=O)c1ccc(CN(C)C)cc1] |
| 131a | 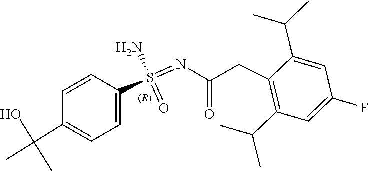 | 184 | 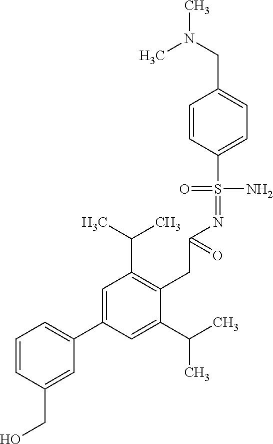 [SMILES: CC(C)c1cc(-c2cccc(CO)c2)cc(C(C)C)c1CC(=O)N=S(N)(=O)c1ccc(CN(C)C)cc1] |
| 131b | 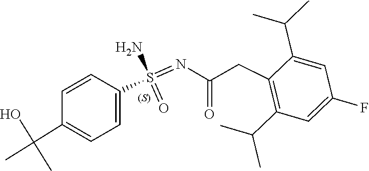 | 185 | 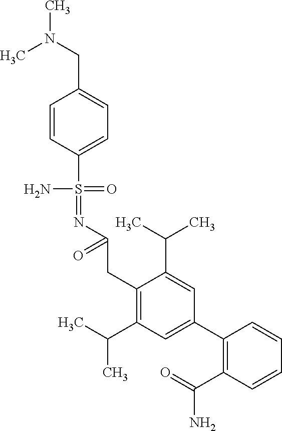 [SMILES: CC(C)c1cc(-c2ccccc2C(N)=O)cc(C(C)C)c1CC(=O)N=S(N)(=O)c1ccc(CN(C)C)cc1] |
| 132 | 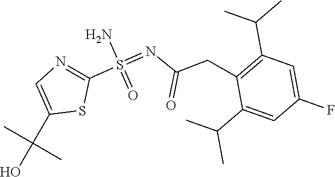 [SMILES: CC(C)c1cc(F)cc(C(C)C)c1CC(=O)N=S(N)(=O)c1ncc(C(C)(C)O)s1] | 186 | 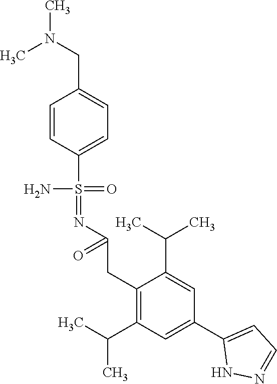 [SMILES: CC(C)c1cc(-c2ccn[nH]2)cc(C(C)C)c1CC(=O)N=S(N)(=O)c1ccc(CN(C)C)cc1] |
| 132a | 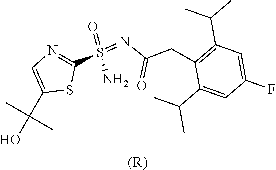 [SMILES: CC(C)c1cc(F)cc(C(C)C)c1CC(=O)N=[S@](N)(=O)c1ncc(C(C)(C)O)s1 ; "(R)"] | 187 | 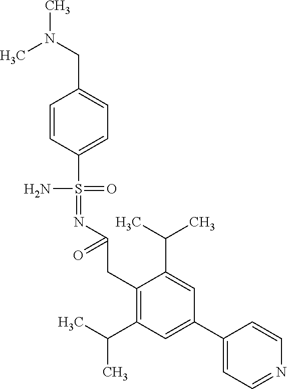 [SMILES: CC(C)c1cc(-c2ccncc2)cc(C(C)C)c1CC(=O)N=S(N)(=O)c1ccc(CN(C)C)cc1] |
| 132b | 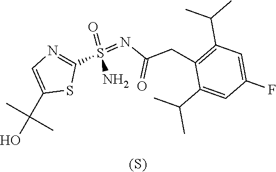 [SMILES: CC(C)c1cc(F)cc(C(C)C)c1CC(=O)N=[S@@](N)(=O)c1ncc(C(C)(C)O)s1 ; "(S)"] | 188 | 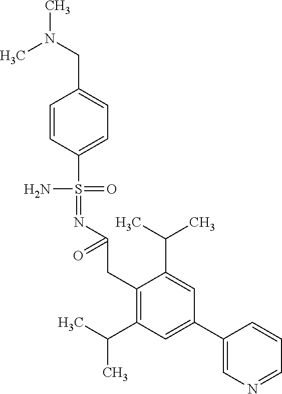 [SMILES: CC(C)c1cc(-c2cccnc2)cc(C(C)C)c1CC(=O)N=S(N)(=O)c1ccc(CN(C)C)cc1] |
| 133 | 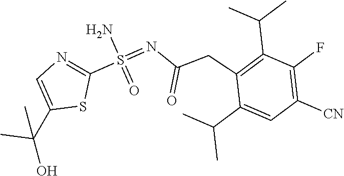 [SMILES: CC(C)c1cc(C#N)c(F)c(C(C)C)c1CC(=O)N=S(N)(=O)c1ncc(C(C)(C)O)s1] | 189 | 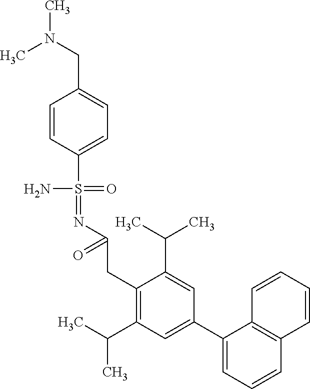 [SMILES: CC(C)c1cc(-c2cccc3ccccc23)cc(C(C)C)c1CC(=O)N=S(N)(=O)c1ccc(CN(C)C)cc1] |
| 133a | 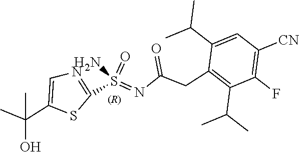 | 190 | 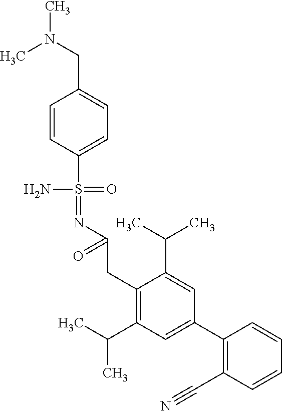 [SMILES: CC(C)c1cc(-c2ccccc2C#N)cc(C(C)C)c1CC(=O)N=S(N)(=O)c1ccc(CN(C)C)cc1] |
| 133b | 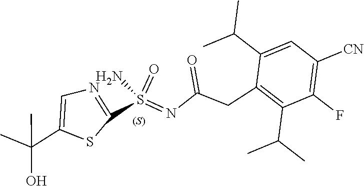 | 191 | 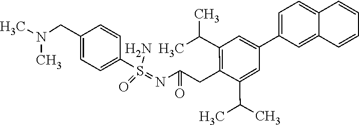 [SMILES: CC(C)c1cc(-c2ccc3ccccc3c2)cc(C(C)C)c1CC(=O)N=S(N)(=O)c1ccc(CN(C)C)cc1] |
| 134 | 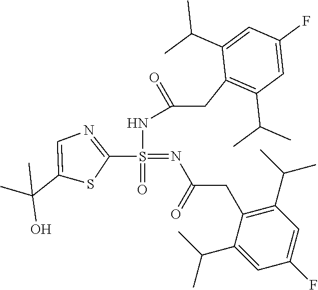 [SMILES: CC(C)c1cc(F)cc(C(C)C)c1CC(=O)N=S(=O)(NC(=O)Cc1c(C(C)C)cc(F)cc1C(C)C)c1ncc(C(C)(C)O)s1] | 192 | 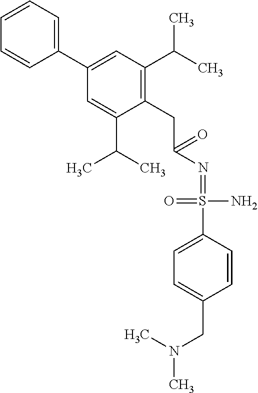 [SMILES: CC(C)c1cc(-c2ccccc2)cc(C(C)C)c1CC(=O)N=S(N)(=O)c1ccc(CN(C)C)cc1] |
| 135 | 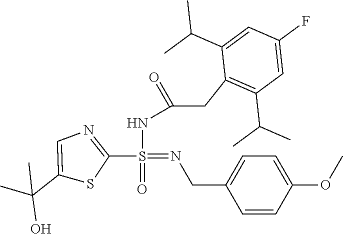 [SMILES: COc1ccc(CN=S(=O)(NC(=O)Cc2c(C(C)C)cc(F)cc2C(C)C)c2ncc(C(C)(C)O)s2)cc1] | 193 | 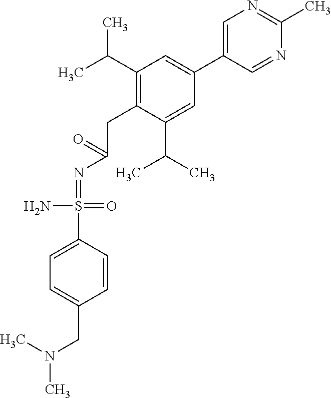 |
| 136 | 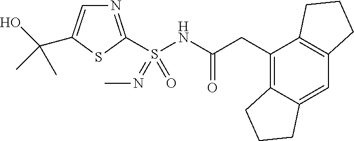 [SMILES: CN=S(=O)(NC(=O)Cc1c2c(cc3c1CCC3)CCC2)c1ncc(C(C)(C)O)s1] | 194 | 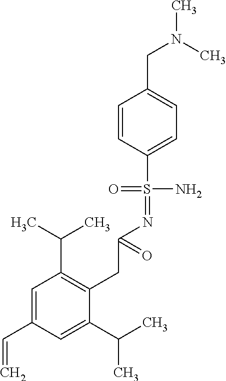 [SMILES: C=Cc1cc(C(C)C)c(CC(=O)N=S(N)(=O)c2ccc(CN(C)C)cc2)c(C(C)C)c1] |
| 137 | 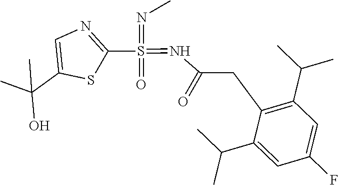 | 195 | 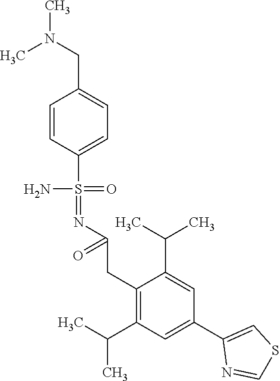 [SMILES: CC(C)c1cc(-c2cscn2)cc(C(C)C)c1CC(=O)N=S(N)(=O)c1ccc(CN(C)C)cc1] |
| 138 | 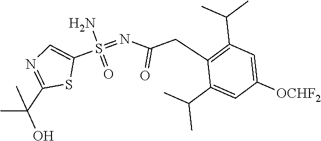 [SMILES: CC(C)c1cc(OC(F)F)cc(C(C)C)c1CC(=O)N=S(N)(=O)c1cnc(C(C)(C)O)s1] | 196 | 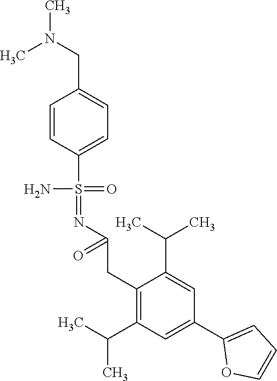 [SMILES: CC(C)c1cc(-c2ccco2)cc(C(C)C)c1CC(=O)N=S(N)(=O)c1ccc(CN(C)C)cc1] |
| 138a | 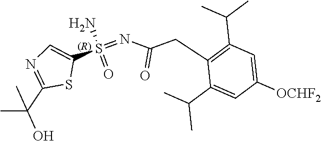 | 197 | 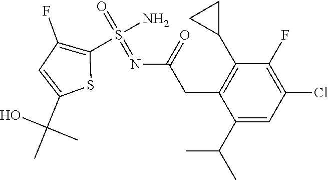 [SMILES: CC(C)c1cc(Cl)c(F)c(C2CC2)c1CC(=O)N=S(N)(=O)c1sc(C(C)(C)O)cc1F] |
| 138b | 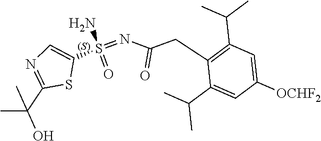 | 197a | 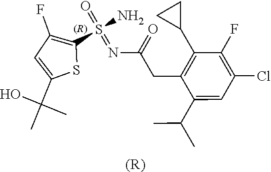 |
| 139 | 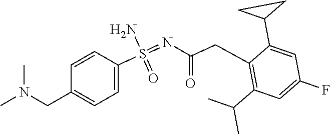 [SMILES: CC(C)c1cc(F)cc(C2CC2)c1CC(=O)N=S(N)(=O)c1ccc(CN(C)C)cc1] | 197b | 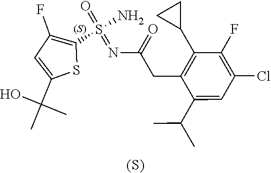 |
| 139a | 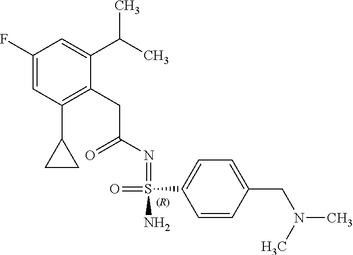 | 198 | 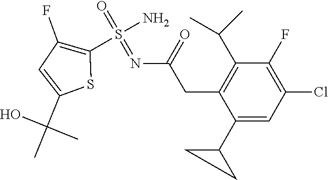 [SMILES: CC(C)c1c(F)c(Cl)cc(C2CC2)c1CC(=O)N=S(N)(=O)c1sc(C(C)(C)O)cc1F] |
| 139b | 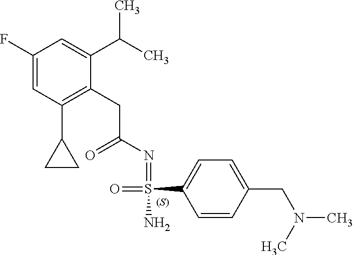 | 198a | 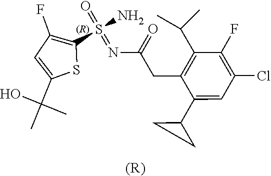 |
| 140 | 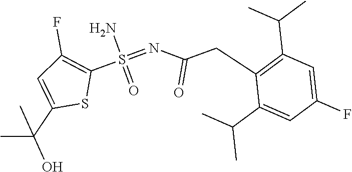 [SMILES: CC(C)c1cc(F)cc(C(C)C)c1CC(=O)N=S(N)(=O)c1sc(C(C)(C)O)cc1F] | 198b | 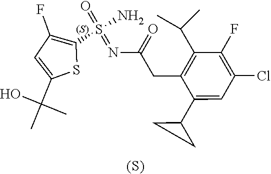 |
| 140a | 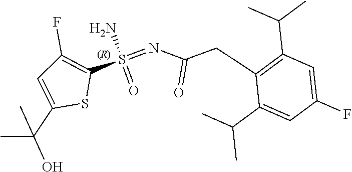 | | |
| 201 | 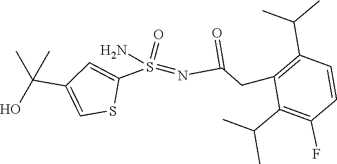 [SMILES: CC(C)c1ccc(F)c(C(C)C)c1CC(=O)N=S(N)(=O)c1cc(C(C)(C)O)cs1] | 231 | 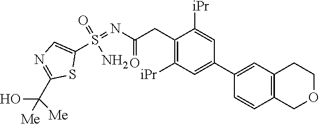 [SMILES: CC(C)c1cc(-c2ccc3c(c2)CCOC3)cc(C(C)C)c1CC(=O)N=S(N)(=O)c1cnc(C(C)(C)O)s1] |
| 201b | 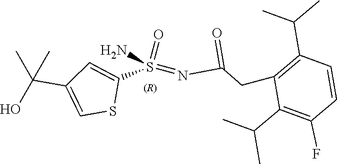 | 232 | 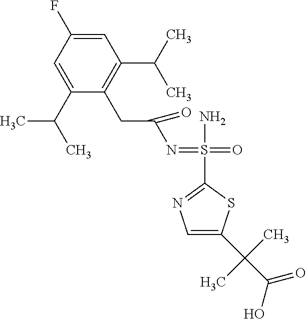 [SMILES: CC(C)c1cc(F)cc(C(C)C)c1CC(=O)N=S(N)(=O)c1ncc(C(C)(C)C(=O)O)s1] |
| 201a | 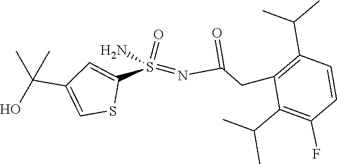 [SMILES: CC(C)c1ccc(F)c(C(C)C)c1CC(=O)N=[S@](N)(=O)c1cc(C(C)(C)O)cs1] | 233 | 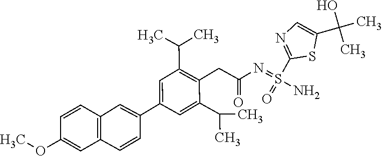 [SMILES: COc1ccc2cc(-c3cc(C(C)C)c(CC(=O)N=S(N)(=O)c4ncc(C(C)(C)O)s4)c(C(C)C)c3)ccc2c1] |
| 202 | 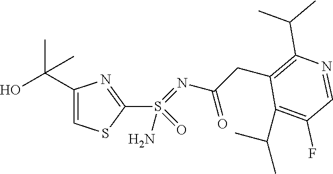 [SMILES: CC(C)c1ncc(F)c(C(C)C)c1CC(=O)N=S(N)(=O)c1nc(C(C)(C)O)cs1] | 234 | 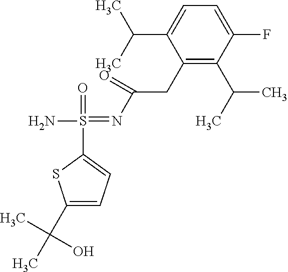 [SMILES: CC(C)c1ccc(F)c(C(C)C)c1CC(=O)N=S(N)(=O)c1ccc(C(C)(C)O)s1] |
| 202b | 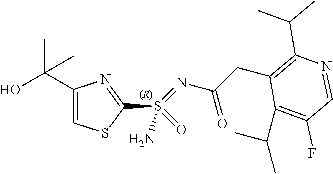 | 234a | 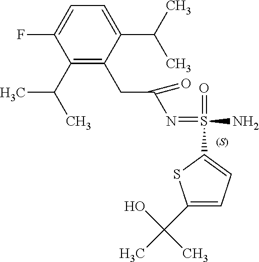 |
| 202a | 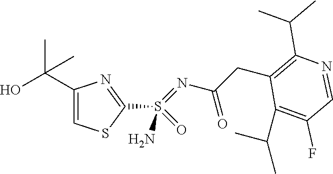 [SMILES: CC(C)c1ncc(F)c(C(C)C)c1CC(=O)N=[S@](N)(=O)c1nc(C(C)(C)O)cs1] | 234b | 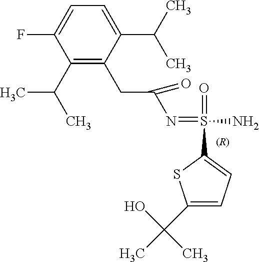 |
| 203 | 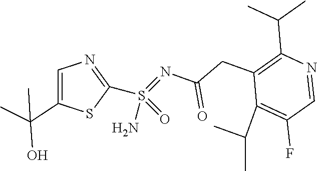 [SMILES: CC(C)c1ncc(F)c(C(C)C)c1CC(=O)N=S(N)(=O)c1ncc(C(C)(C)O)s1] | 235 | 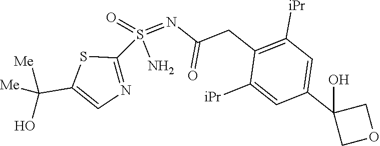 [SMILES: CC(C)c1cc(C2(O)COC2)cc(C(C)C)c1CC(=O)N=S(N)(=O)c1ncc(C(C)(C)O)s1] |
| 203b | 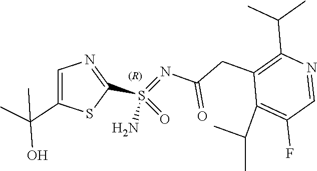 | 236 | 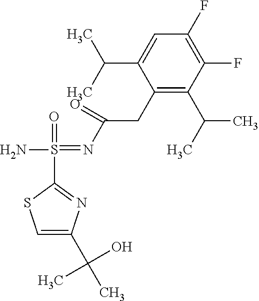 [SMILES: CC(C)c1cc(F)c(F)c(C(C)C)c1CC(=O)N=S(N)(=O)c1nc(C(C)(C)O)cs1] |
| 203a | 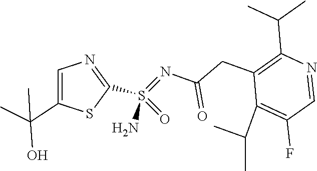 [SMILES: CC(C)c1ncc(F)c(C(C)C)c1CC(=O)N=[S@](N)(=O)c1ncc(C(C)(C)O)s1] | 236a | 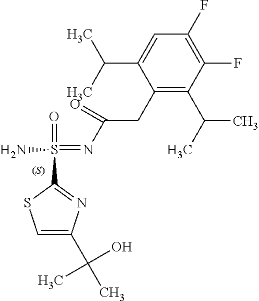 |
| 204 | 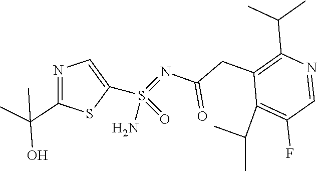 [SMILES: CC(C)c1ncc(F)c(C(C)C)c1CC(=O)N=S(N)(=O)c1cnc(C(C)(C)O)s1] | 236b | 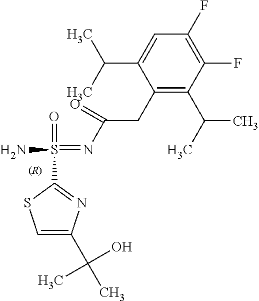 |
| 204b | 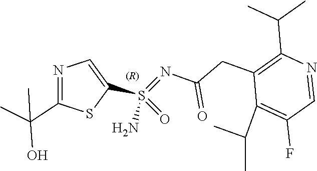 | 237 | 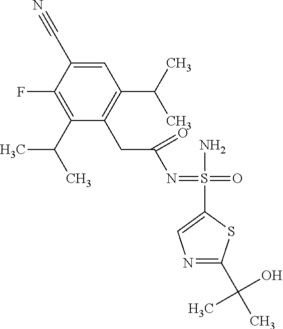 [SMILES: CC(C)c1cc(C#N)c(F)c(C(C)C)c1CC(=O)N=S(N)(=O)c1cnc(C(C)(C)O)s1] |
| 204a | 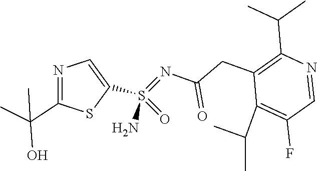 [SMILES: CC(C)c1ncc(F)c(C(C)C)c1CC(=O)N=[S@](N)(=O)c1cnc(C(C)(C)O)s1] | 238 | 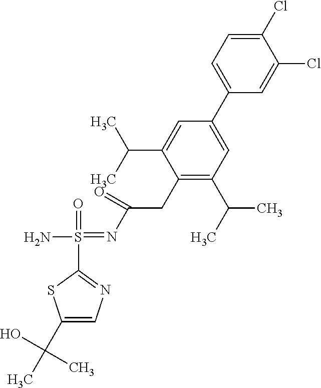 [SMILES: CC(C)c1cc(-c2ccc(Cl)c(Cl)c2)cc(C(C)C)c1CC(=O)N=S(N)(=O)c1ncc(C(C)(C)O)s1] |
| 205 | 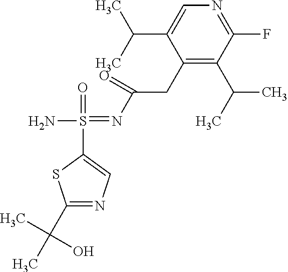 [SMILES: CC(C)c1cnc(F)c(C(C)C)c1CC(=O)N=S(N)(=O)c1cnc(C(C)(C)O)s1] | 238a | 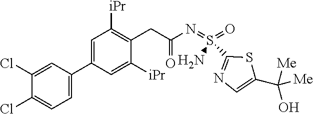 [SMILES: CC(C)c1cc(-c2ccc(Cl)c(Cl)c2)cc(C(C)C)c1CC(=O)N=[S@](N)(=O)c1ncc(C(C)(C)O)s1] |
| 206 | 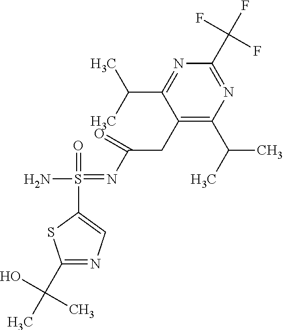 [SMILES: CC(C)c1nc(C(F)(F)F)nc(C(C)C)c1CC(=O)N=S(N)(=O)c1cnc(C(C)(C)O)s1] | 238b | 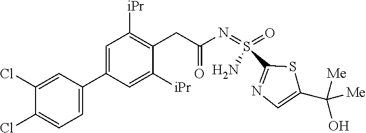 [SMILES: CC(C)c1cc(-c2ccc(Cl)c(Cl)c2)cc(C(C)C)c1CC(=O)N=[S@@](N)(=O)c1ncc(C(C)(C)O)s1] |
| 207 | 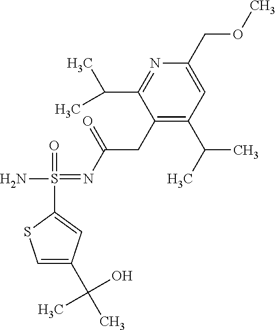 [SMILES: COCc1cc(C(C)C)c(CC(=O)N=S(N)(=O)c2cc(C(C)(C)O)cs2)c(C(C)C)n1] | 239 | 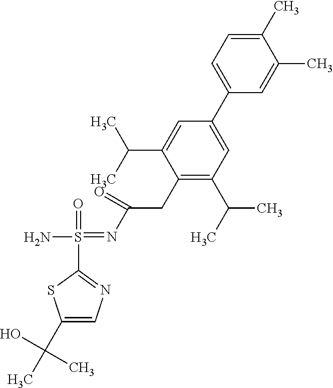 [SMILES: Cc1ccc(-c2cc(C(C)C)c(CC(=O)N=S(N)(=O)c3ncc(C(C)(C)O)s3)c(C(C)C)c2)cc1C] |
| 208 | 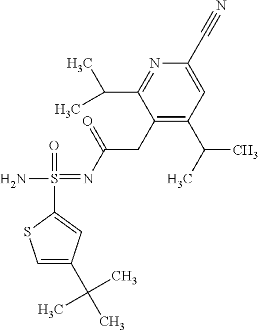 [SMILES: CC(C)c1cc(C#N)nc(C(C)C)c1CC(=O)N=S(N)(=O)c1cc(C(C)(C)C)cs1] | 239a | 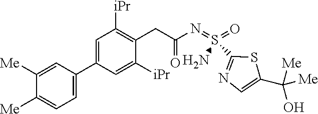 [SMILES: Cc1ccc(-c2cc(C(C)C)c(CC(=O)N=[S@](N)(=O)c3ncc(C(C)(C)O)s3)c(C(C)C)c2)cc1C] |
| 209 | 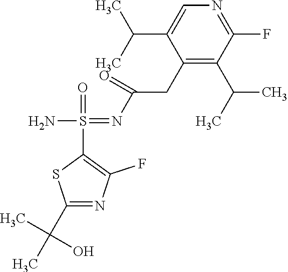 [SMILES: CC(C)c1cnc(F)c(C(C)C)c1CC(=O)N=S(N)(=O)c1sc(C(C)(C)O)nc1F] | 239b | 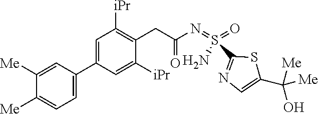 [SMILES: Cc1ccc(-c2cc(C(C)C)c(CC(=O)N=[S@@](N)(=O)c3ncc(C(C)(C)O)s3)c(C(C)C)c2)cc1C] |
| 210 | 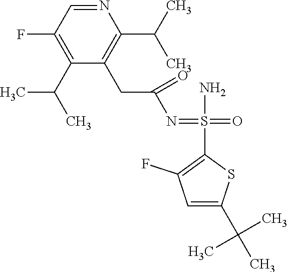 [SMILES: CC(C)c1ncc(F)c(C(C)C)c1CC(=O)N=S(N)(=O)c1sc(C(C)(C)C)cc1F] | 240 | 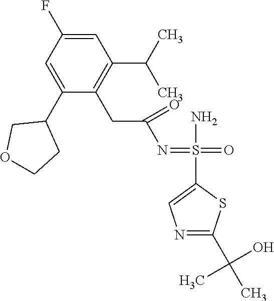 [SMILES: CC(C)c1cc(F)cc(C2CCOC2)c1CC(=O)N=S(N)(=O)c1cnc(C(C)(C)O)s1] |
| 210a | 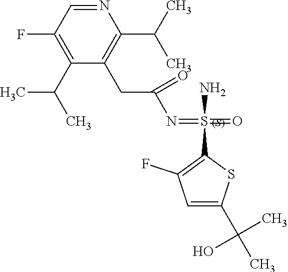 | 241 | 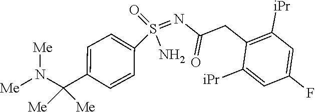 [SMILES: CC(C)c1cc(F)cc(C(C)C)c1CC(=O)N=S(N)(=O)c1ccc(C(C)(C)N(C)C)cc1] |
| 210b | 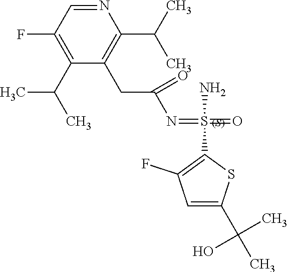 | 242 | 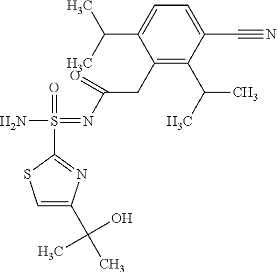 [SMILES: CC(C)c1ccc(C#N)c(C(C)C)c1CC(=O)N=S(N)(=O)c1nc(C(C)(C)O)cs1] |
| 211 | 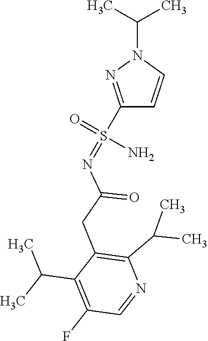 [SMILES: CC(C)c1ncc(F)c(C(C)C)c1CC(=O)N=S(N)(=O)c1ccn(C(C)C)n1] | 242a | 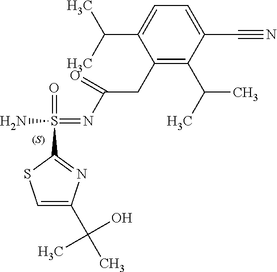 |
| 212 | 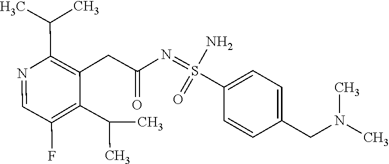 [SMILES: CC(C)c1ncc(F)c(C(C)C)c1CC(=O)N=S(N)(=O)c1ccc(CN(C)C)cc1] | 242b | 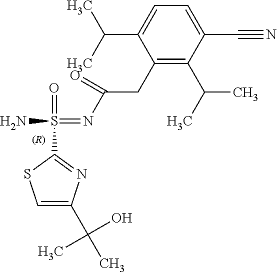 |
| 213 | 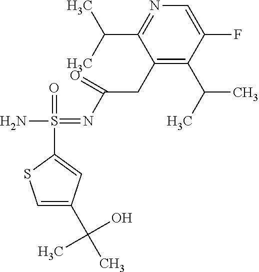 [SMILES: CC(C)c1ncc(F)c(C(C)C)c1CC(=O)N=S(N)(=O)c1cc(C(C)(C)O)cs1] | 243 | 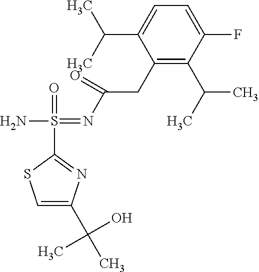 [SMILES: CC(C)c1ccc(F)c(C(C)C)c1CC(=O)N=S(N)(=O)c1nc(C(C)(C)O)cs1] |
| 213a | 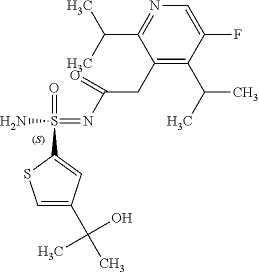 | 243a | 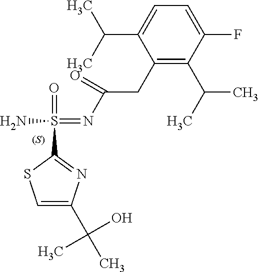 |
| 213b | 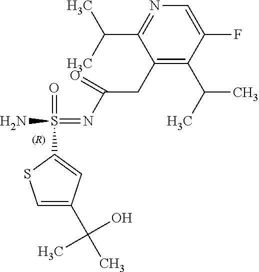 | 243b | 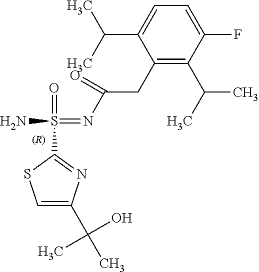 |
| 214 | 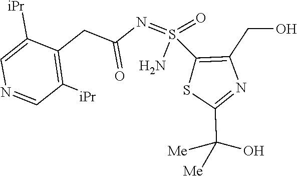 [SMILES: CC(C)c1cncc(C(C)C)c1CC(=O)N=S(N)(=O)c1sc(C(C)(C)O)nc1CO] | 244 | 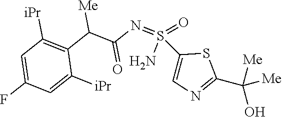 [SMILES: CC(C)c1cc(F)cc(C(C)C)c1C(C)C(=O)N=S(N)(=O)c1cnc(C(C)(C)O)s1] |
| 215 | 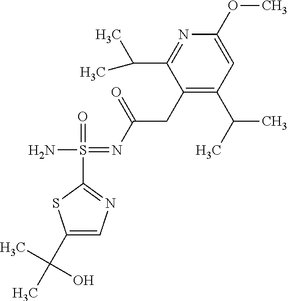 [SMILES: COc1cc(C(C)C)c(CC(=O)N=S(N)(=O)c2ncc(C(C)(C)O)s2)c(C(C)C)n1] | 244aa | 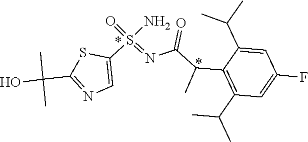 [SMILES: CC(C)c1cc(F)cc(C(C)C)c1C(C)C(=O)N=S(N)(=O)c1cnc(C(C)(C)O)s1] |
| 216 | 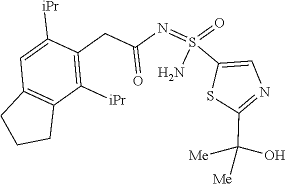 [SMILES: CC(C)c1cc2c(c(C(C)C)c1CC(=O)N=S(N)(=O)c1cnc(C(C)(C)O)s1)CCC2] | 244b | 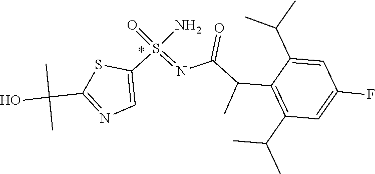 [SMILES: CC(C)c1cc(F)cc(C(C)C)c1C(C)C(=O)N=S(N)(=O)c1cnc(C(C)(C)O)s1] |
| 216a | 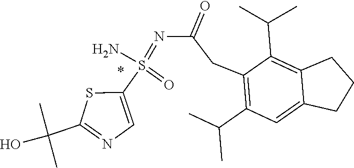 [SMILES: CC(C)c1cc2c(c(C(C)C)c1CC(=O)N=S(N)(=O)c1cnc(C(C)(C)O)s1)CCC2] | 244ba | 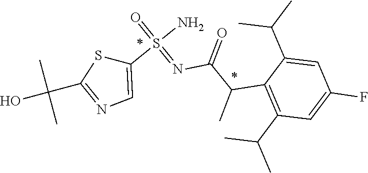 [SMILES: CC(C)c1cc(F)cc(C(C)C)c1C(C)C(=O)N=S(N)(=O)c1cnc(C(C)(C)O)s1] |
| 216b | 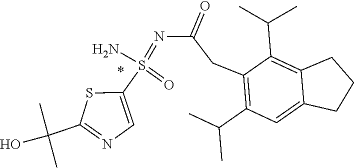 [SMILES: CC(C)c1cc2c(c(C(C)C)c1CC(=O)N=S(N)(=O)c1cnc(C(C)(C)O)s1)CCC2] | 245 | 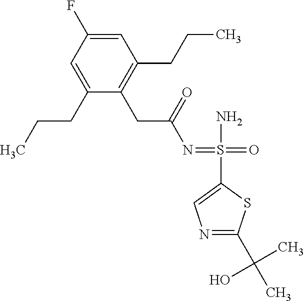 [SMILES: CCCc1cc(F)cc(CCC)c1CC(=O)N=S(N)(=O)c1cnc(C(C)(C)O)s1] |
| 217 | 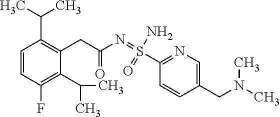 [SMILES: CC(C)c1ccc(F)c(C(C)C)c1CC(=O)N=S(N)(=O)c1ccc(CN(C)C)cn1] | 246 | 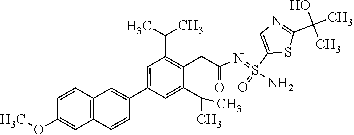 [SMILES: COc1ccc2cc(-c3cc(C(C)C)c(CC(=O)N=S(N)(=O)c4cnc(C(C)(C)O)s4)c(C(C)C)c3)ccc2c1] |
| 217a | 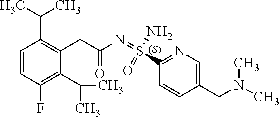 | 247 | 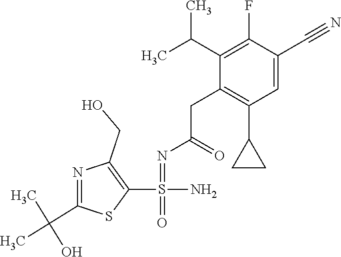 [SMILES: CC(C)c1c(F)c(C#N)cc(C2CC2)c1CC(=O)N=S(N)(=O)c1sc(C(C)(C)O)nc1CO] |
| 217b | 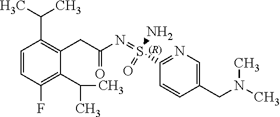 | 248 | 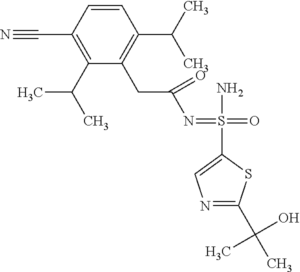 [SMILES: CC(C)c1ccc(C#N)c(C(C)C)c1CC(=O)N=S(N)(=O)c1cnc(C(C)(C)O)s1] |
| 218 | 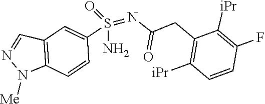 [SMILES: CC(C)c1ccc(F)c(C(C)C)c1CC(=O)N=S(N)(=O)c1ccc2c(cnn2C)c1] | 249 | 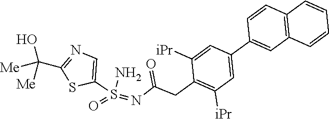 [SMILES: CC(C)c1cc(-c2ccc3ccccc3c2)cc(C(C)C)c1CC(=O)N=S(N)(=O)c1cnc(C(C)(C)O)s1] |
| 218a | 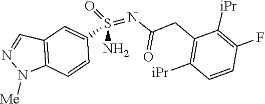 [SMILES: CC(C)c1ccc(F)c(C(C)C)c1CC(=O)N=[S@@](N)(=O)c1ccc2c(cnn2C)c1] | 249a | 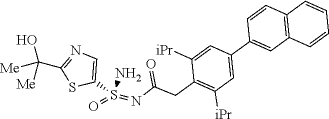 [SMILES: CC(C)c1cc(-c2ccc3ccccc3c2)cc(C(C)C)c1CC(=O)N=[S@](N)(=O)c1cnc(C(C)(C)O)s1] |
| 218b | 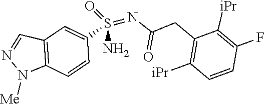 [SMILES: CC(C)c1ccc(F)c(C(C)C)c1CC(=O)N=[S@@](N)(=O)c1ccc2c(cnn2C)c1] | 249b | 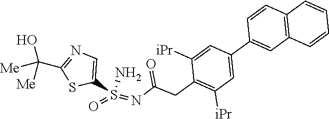 [SMILES: CC(C)c1cc(-c2ccc3ccccc3c2)cc(C(C)C)c1CC(=O)N=[S@@](N)(=O)c1cnc(C(C)(C)O)s1] |
| 218b | 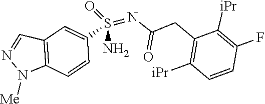 [SMILES: CC(C)c1ccc(F)c(C(C)C)c1CC(=O)N=[S@@](N)(=O)c1ccc2c(cnn2C)c1] | 249b | 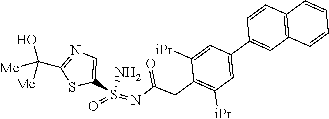 [SMILES: CC(C)c1cc(-c2ccc3ccccc3c2)cc(C(C)C)c1CC(=O)N=[S@@](N)(=O)c1cnc(C(C)(C)O)s1] |
| 219 | 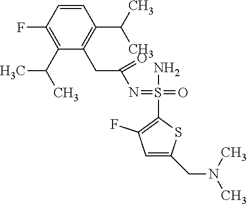 [SMILES: CC(C)c1ccc(F)c(C(C)C)c1CC(=O)N=S(N)(=O)c1sc(CN(C)C)cc1F] | 250 | 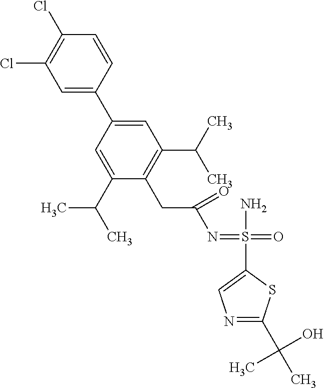 [SMILES: CC(C)c1cc(-c2ccc(Cl)c(Cl)c2)cc(C(C)C)c1CC(=O)N=S(N)(=O)c1cnc(C(C)(C)O)s1] |
| 220 | 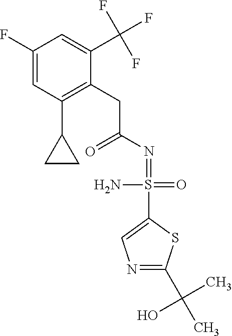 [SMILES: CC(C)(O)c1ncc(S(N)(=O)=NC(=O)Cc2c(C3CC3)cc(F)cc2C(F)(F)F)s1] | 251 | 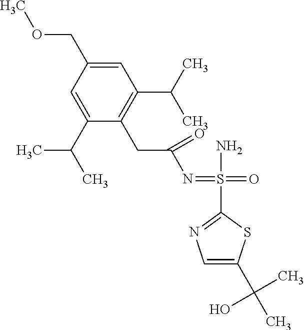 [SMILES: COCc1cc(C(C)C)c(CC(=O)N=S(N)(=O)c2ncc(C(C)(C)O)s2)c(C(C)C)c1] |
| 220a | 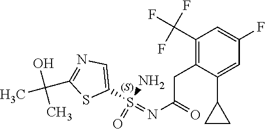 | 251a | 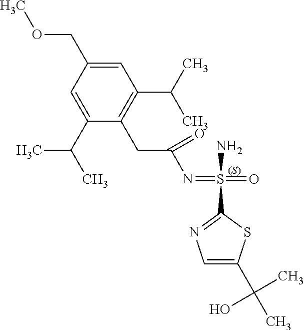 |
| 220b | 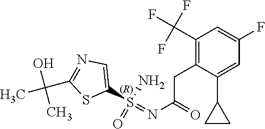 | 251b | 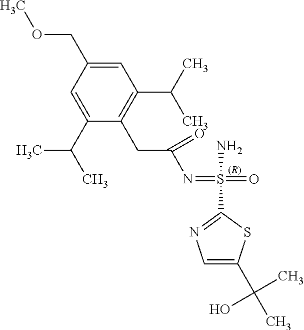 |
| 221 | 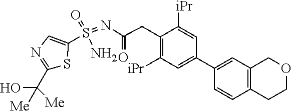 [SMILES: CC(C)c1cc(-c2ccc3c(c2)COCC3)cc(C(C)C)c1CC(=O)N=S(N)(=O)c1cnc(C(C)(C)O)s1] | 252 | 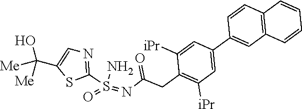 [SMILES: CC(C)c1cc(-c2ccc3ccccc3c2)cc(C(C)C)c1CC(=O)N=S(N)(=O)c1ncc(C(C)(C)O)s1] |
| 221a | 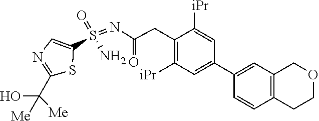 [SMILES: CC(C)c1cc(-c2ccc3c(c2)COCC3)cc(C(C)C)c1CC(=O)N=[S@](N)(=O)c1cnc(C(C)(C)O)s1] | 253 | 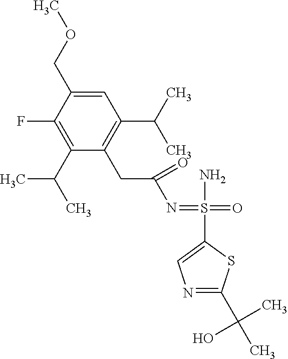 [SMILES: COCc1cc(C(C)C)c(CC(=O)N=S(N)(=O)c2cnc(C(C)(C)O)s2)c(C(C)C)c1F] |
| 221b | 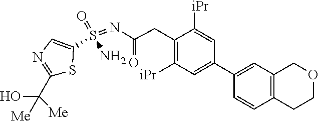 [SMILES: CC(C)c1cc(-c2ccc3c(c2)COCC3)cc(C(C)C)c1CC(=O)N=[S@@](N)(=O)c1cnc(C(C)(C)O)s1] | 254 | 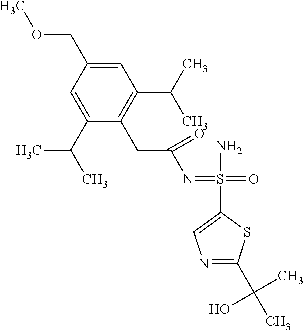 [SMILES: COCc1cc(C(C)C)c(CC(=O)N=S(N)(=O)c2cnc(C(C)(C)O)s2)c(C(C)C)c1] |
| 222 | 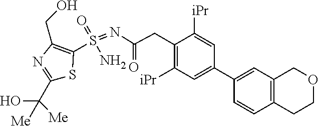 [SMILES: CC(C)c1cc(-c2ccc3c(c2)COCC3)cc(C(C)C)c1CC(=O)N=S(N)(=O)c1sc(C(C)(C)O)nc1CO] | 255 | 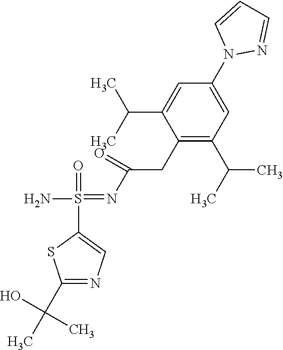 [SMILES: CC(C)c1cc(-n2cccn2)cc(C(C)C)c1CC(=O)N=S(N)(=O)c1cnc(C(C)(C)O)s1] |
| 224 | 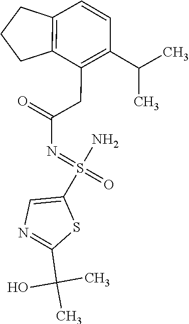 [SMILES: CC(C)c1ccc2c(c1CC(=O)N=S(N)(=O)c1cnc(C(C)(C)O)s1)CCC2] | 256 | 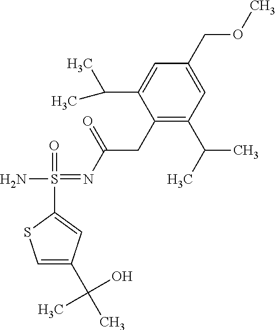 [SMILES: COCc1cc(C(C)C)c(CC(=O)N=S(N)(=O)c2cc(C(C)(C)O)cs2)c(C(C)C)c1] |
| 224b | 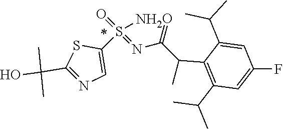 [SMILES: CC(C)c1cc(F)cc(C(C)C)c1C(C)C(=O)N=S(N)(=O)c1cnc(C(C)(C)O)s1] | 256a | 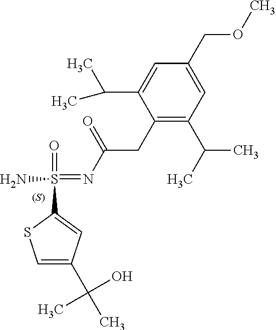 |
| 224ba | 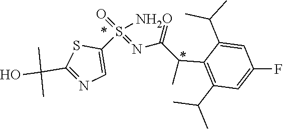 [SMILES: CC(C)c1cc(F)cc(C(C)C)c1C(C)C(=O)N=S(N)(=O)c1cnc(C(C)(C)O)s1] | 256b | 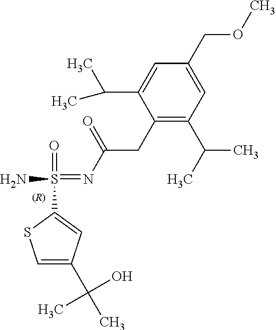 |
| 224aa | 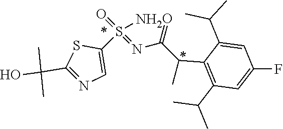 [SMILES: CC(C)c1cc(F)cc(C(C)C)c1C(C)C(=O)N=S(N)(=O)c1cnc(C(C)(C)O)s1] | 257 | 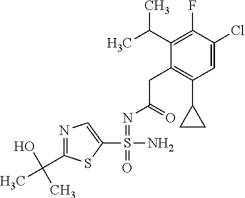 [SMILES: CC(C)c1c(F)c(Cl)cc(C2CC2)c1CC(=O)N=S(N)(=O)c1cnc(C(C)(C)O)s1] |
| 225 | 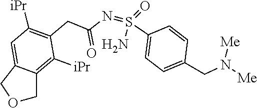 [SMILES: CC(C)c1cc2c(c(C(C)C)c1CC(=O)N=S(N)(=O)c1ccc(CN(C)C)cc1)COC2] | 258 | 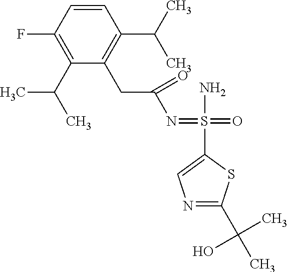 [SMILES: CC(C)c1ccc(F)c(C(C)C)c1CC(=O)N=S(N)(=O)c1cnc(C(C)(C)O)s1] |
| 225a | 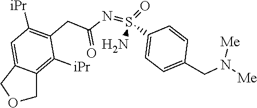 [SMILES: CC(C)c1cc2c(c(C(C)C)c1CC(=O)N=[S@](N)(=O)c1ccc(CN(C)C)cc1)COC2] | 258a | 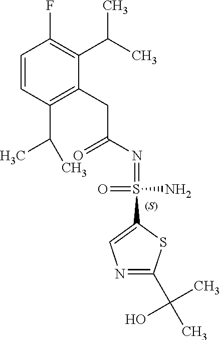 |
| 225b | 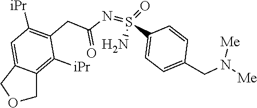 [SMILES: CC(C)c1cc2c(c(C(C)C)c1CC(=O)N=[S@@](N)(=O)c1ccc(CN(C)C)cc1)COC2] | 258b | 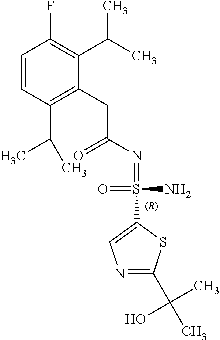 |
| 226 | 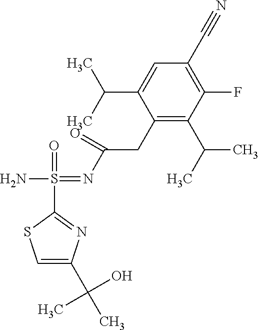 [SMILES: CC(C)c1cc(C#N)c(F)c(C(C)C)c1CC(=O)N=S(N)(=O)c1nc(C(C)(C)O)cs1] | 259 | 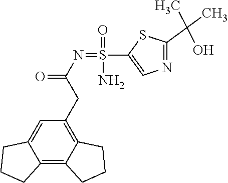 [SMILES: CC(C)(O)c1ncc(S(N)(=O)=NC(=O)Cc2cc3c(c4c2CCC4)CCC3)s1] |
| 226a | 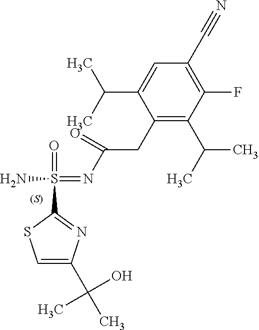 | 259b | 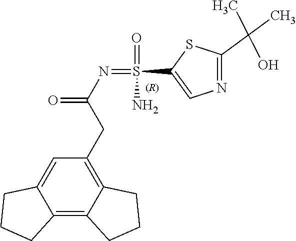 |
| 227 | 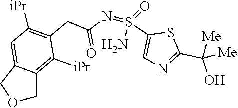 [SMILES: CC(C)c1cc2c(c(C(C)C)c1CC(=O)N=S(N)(=O)c1cnc(C(C)(C)O)s1)COC2] | 260 | 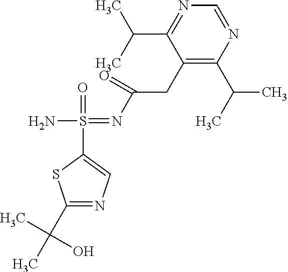 [SMILES: CC(C)c1ncnc(C(C)C)c1CC(=O)N=S(N)(=O)c1cnc(C(C)(C)O)s1] |
| 227a | 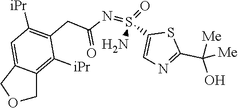 [SMILES: CC(C)c1cc2c(c(C(C)C)c1CC(=O)N=[S@](N)(=O)c1cnc(C(C)(C)O)s1)COC2] | 261a | 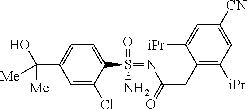 [SMILES: CC(C)c1cc(C#N)cc(C(C)C)c1CC(=O)N=[S@](N)(=O)c1ccc(C(C)(C)O)cc1Cl] |
| 227b | 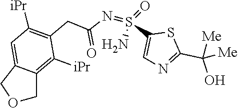 [SMILES: CC(C)c1cc2c(c(C(C)C)c1CC(=O)N=[S@@](N)(=O)c1cnc(C(C)(C)O)s1)COC2] | 261b | 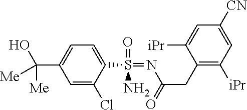 [SMILES: CC(C)c1cc(C#N)cc(C(C)C)c1CC(=O)N=[S@@](N)(=O)c1ccc(C(C)(C)O)cc1Cl] |
| 228 | 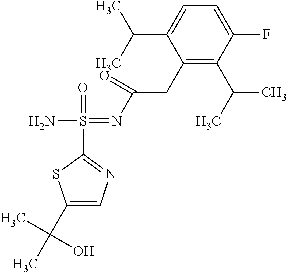 [SMILES: CC(C)c1ccc(F)c(C(C)C)c1CC(=O)N=S(N)(=O)c1ncc(C(C)(C)O)s1] | 262a | 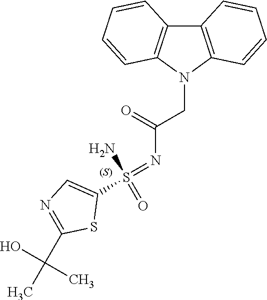 |
| 228a | 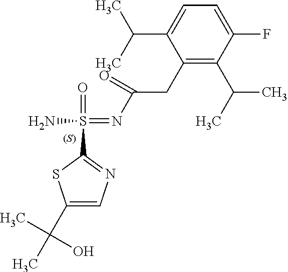 | 262b | 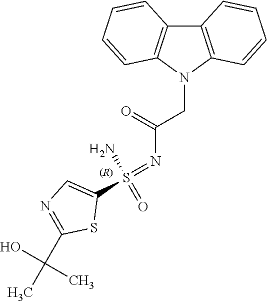 |
| 228b | 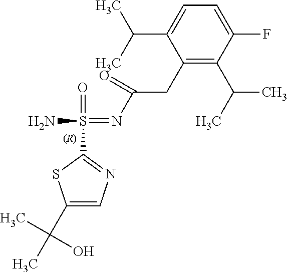 | 263 | 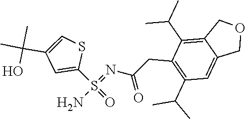 [SMILES: CC(C)c1cc2c(c(C(C)C)c1CC(=O)N=S(N)(=O)c1cc(C(C)(C)O)cs1)COC2] |
| 229 | 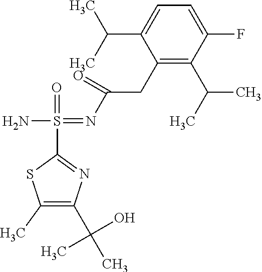 [SMILES: Cc1sc(S(N)(=O)=NC(=O)Cc2c(C(C)C)ccc(F)c2C(C)C)nc1C(C)(C)O] | 263a | 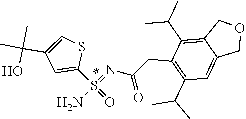 [SMILES: CC(C)c1cc2c(c(C(C)C)c1CC(=O)N=S(N)(=O)c1cc(C(C)(C)O)cs1)COC2] |
| 229a | 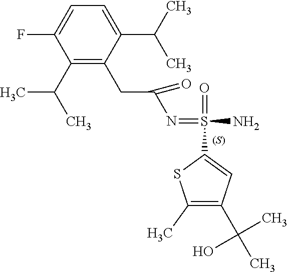 | 263b | 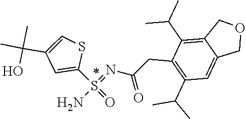 [SMILES: CC(C)c1cc2c(c(C(C)C)c1CC(=O)N=S(N)(=O)c1cc(C(C)(C)O)cs1)COC2] |
| 229b | 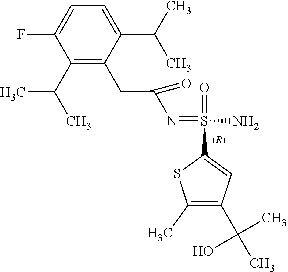 | 264 | 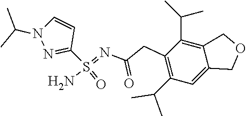 [SMILES: CC(C)c1cc2c(c(C(C)C)c1CC(=O)N=S(N)(=O)c1ccn(C(C)C)n1)COC2] |
| 230 | 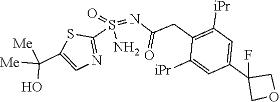 [SMILES: CC(C)c1cc(C2(F)COC2)cc(C(C)C)c1CC(=O)N=S(N)(=O)c1ncc(C(C)(C)O)s1] | 264a | 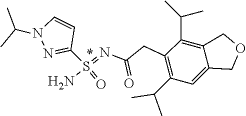 [SMILES: CC(C)c1cc2c(c(C(C)C)c1CC(=O)N=S(N)(=O)c1ccn(C(C)C)n1)COC2] |
| 265 | 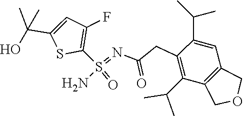 [SMILES: CC(C)c1cc2c(c(C(C)C)c1CC(=O)N=S(N)(=O)c1sc(C(C)(C)O)cc1F)COC2] | 264b | 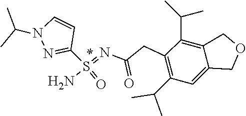 [SMILES: CC(C)c1cc2c(c(C(C)C)c1CC(=O)N=S(N)(=O)c1ccn(C(C)C)n1)COC2] |
| 265a | 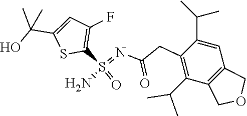 [SMILES: CC(C)c1cc2c(c(C(C)C)c1CC(=O)N=[S@@](N)(=O)c1sc(C(C)(C)O)cc1F)COC2] | | |

and pharmaceutically acceptable salts thereof.

In some embodiments, provided herein is a compound that is selected from the group consisting of the compounds in Table 1-3:

TABLE 1-3

| | | | |
|---|---|---|---|
| 301 | 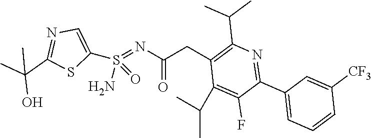 [SMILES: CC(C)c1nc(-c2cccc(C(F)(F)F)c2)c(F)c(C(C)C)c1CC(=O)N=S(N)(=O)c1cnc(C(C)(C)O)s1] | 306 | 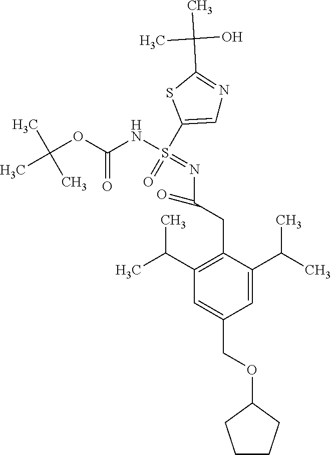 [SMILES: CC(C)c1cc(COC2CCCC2)cc(C(C)C)c1CC(=O)N=S(=O)(NC(=O)OC(C)(C)C)c1cnc(C(C)(C)O)s1] |
| 302 | 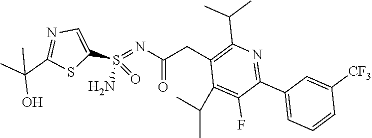 [SMILES: CC(C)c1nc(-c2cccc(C(F)(F)F)c2)c(F)c(C(C)C)c1CC(=O)N=[S@@](N)(=O)c1cnc(C(C)(C)O)s1] | 307 | 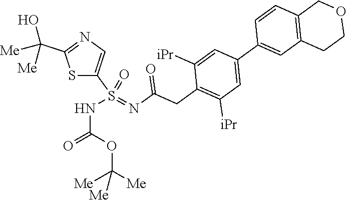 [SMILES: CC(C)c1cc(-c2ccc3c(c2)CCOC3)cc(C(C)C)c1CC(=O)N=S(=O)(NC(=O)OC(C)(C)C)c1cnc(C(C)(C)O)s1] |
| 303 | 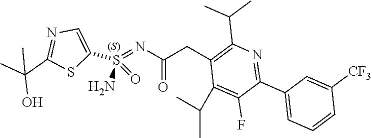 | 309 | 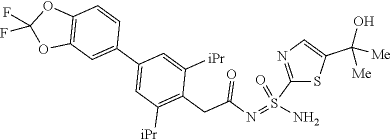 [SMILES: CC(C)c1cc(-c2ccc3c(c2)OC(F)(F)O3)cc(C(C)C)c1CC(=O)N=S(N)(=O)c1ncc(C(C)(C)O)s1] |
| 304 | 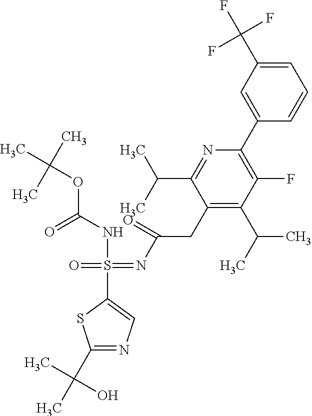 [SMILES: CC(C)c1nc(-c2cccc(C(F)(F)F)c2)c(F)c(C(C)C)c1CC(=O)N=S(=O)(NC(=O)OC(C)(C)C)c1cnc(C(C)(C)O)s1] | 309a | 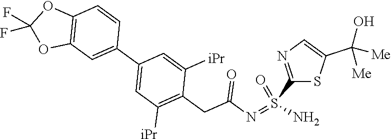 [SMILES: CC(C)c1cc(-c2ccc3c(c2)OC(F)(F)O3)cc(C(C)C)c1CC(=O)N=[S@@](N)(=O)c1ncc(C(C)(C)O)s1] |
| 305 | 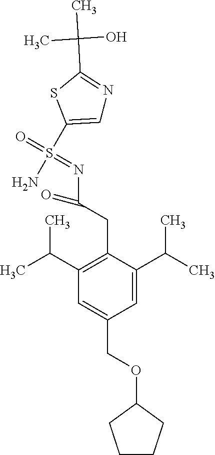 [SMILES: CC(C)c1cc(COC2CCCC2)cc(C(C)C)c1CC(=O)N=S(N)(=O)c1cnc(C(C)(C)O)s1] | 309b | 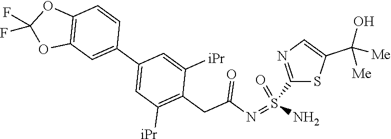 [SMILES: CC(C)c1cc(-c2ccc3c(c2)OC(F)(F)O3)cc(C(C)C)c1CC(=O)N=[S@](N)(=O)c1ncc(C(C)(C)O)s1] |
| | | 310 | 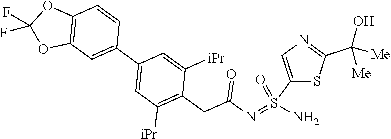 [SMILES: CC(C)c1cc(-c2ccc3c(c2)OC(F)(F)O3)cc(C(C)C)c1CC(=O)N=S(N)(=O)c1cnc(C(C)(C)O)s1] |

or a pharmaceutically acceptable salt thereof.

In some embodiments, provided herein is a compound that is selected from the group consisting of the compounds in Table 1-4:

TABLE 1-4

| | |
|---|---|
| 401 | 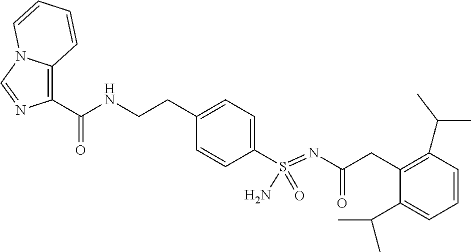 [SMILES: CC(C)c1cccc(C(C)C)c1CC(=O)N=S(N)(=O)c1ccc(CCNC(=O)c2ncn3ccccc23)cc1] |
| 401a | 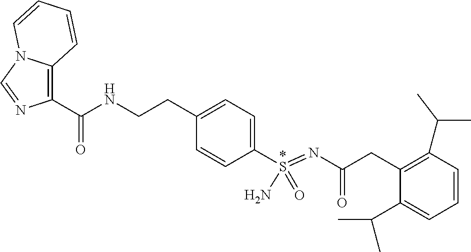 [SMILES: CC(C)c1cccc(C(C)C)c1CC(=O)N=S(N)(=O)c1ccc(CCNC(=O)c2ncn3ccccc23)cc1] |
| 401b | 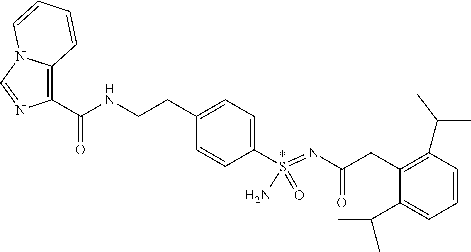 [SMILES: CC(C)c1cccc(C(C)C)c1CC(=O)N=S(N)(=O)c1ccc(CCNC(=O)c2ncn3ccccc23)cc1] |
| 402 | 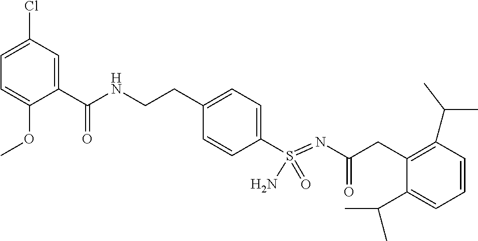 [SMILES: COc1ccc(Cl)cc1C(=O)NCCc1ccc(S(N)(=O)=NC(=O)Cc2c(C(C)C)cccc2C(C)C)cc1] |
| 402a | 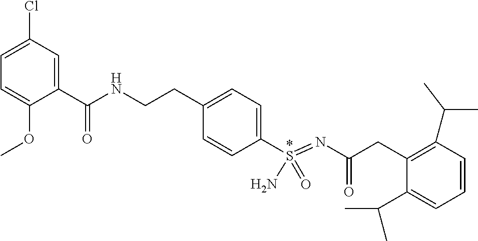 [SMILES: COc1ccc(Cl)cc1C(=O)NCCc1ccc(S(N)(=O)=NC(=O)Cc2c(C(C)C)cccc2C(C)C)cc1] |
| 402b | 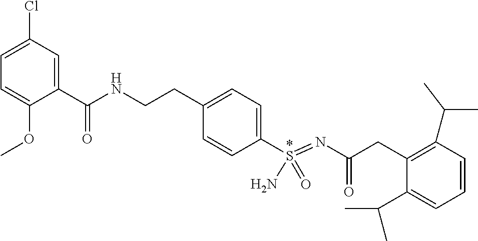 [SMILES: COc1ccc(Cl)cc1C(=O)NCCc1ccc(S(N)(=O)=NC(=O)Cc2c(C(C)C)cccc2C(C)C)cc1] |
| 403 | 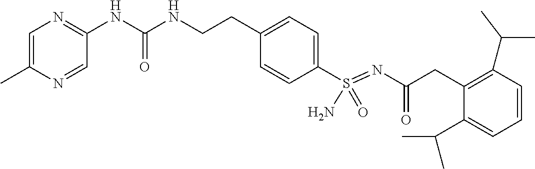 [SMILES: Cc1cnc(NC(=O)NCCc2ccc(S(N)(=O)=NC(=O)Cc3c(C(C)C)cccc3C(C)C)cc2)cn1] |
| 403a | 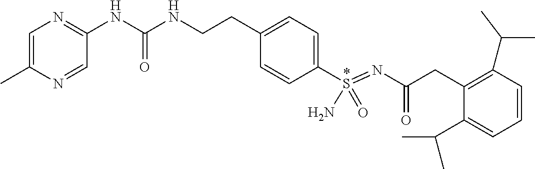 [SMILES: Cc1cnc(NC(=O)NCCc2ccc(S(N)(=O)=NC(=O)Cc3c(C(C)C)cccc3C(C)C)cc2)cn1] |
| 403b | 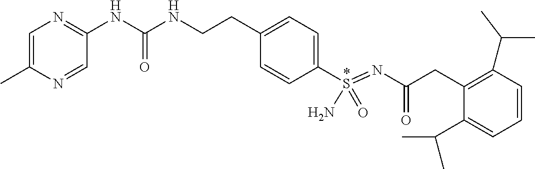 [SMILES: Cc1cnc(NC(=O)NCCc2ccc(S(N)(=O)=NC(=O)Cc3c(C(C)C)cccc3C(C)C)cc2)cn1] |
| 404 | 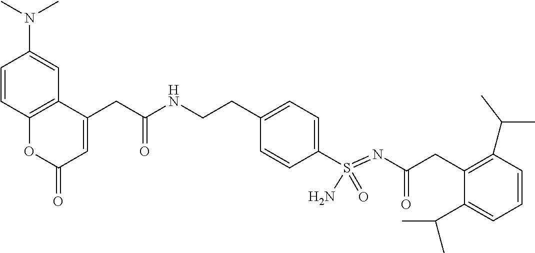 [SMILES: CC(C)c1cccc(C(C)C)c1CC(=O)N=S(N)(=O)c1ccc(CCNC(=O)Cc2cc(=O)oc3ccc(N(C)C)cc23)cc1] |
| 404a | 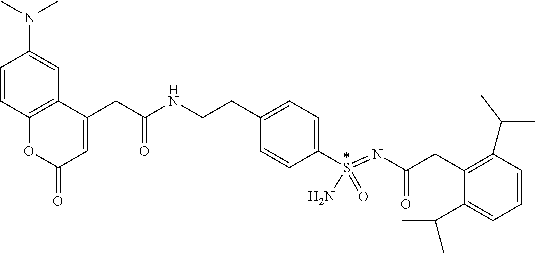 [SMILES: CC(C)c1cccc(C(C)C)c1CC(=O)N=S(N)(=O)c1ccc(CCNC(=O)Cc2cc(=O)oc3ccc(N(C)C)cc23)cc1] |
| 404b | 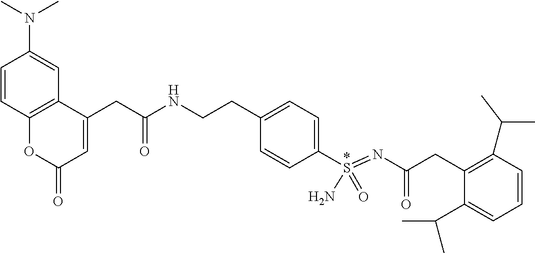 [SMILES: CC(C)c1cccc(C(C)C)c1CC(=O)N=S(N)(=O)c1ccc(CCNC(=O)Cc2cc(=O)oc3ccc(N(C)C)cc23)cc1] |
| 405 | 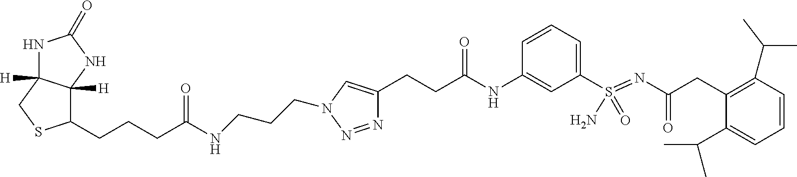 [SMILES: CC(C)c1cccc(C(C)C)c1CC(=O)N=S(N)(=O)c1cccc(NC(=O)CCc2cn(CCCNC(=O)CCCC3SC[C@@H]4NC(=O)N[C@H]34)nn2)c1] |
| 405a | 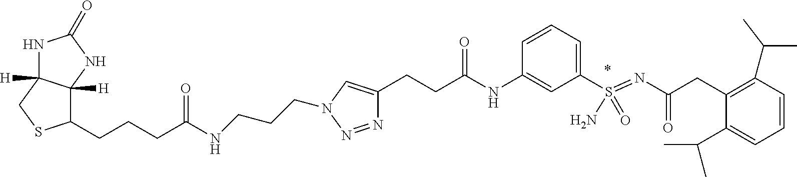 [SMILES: CC(C)c1cccc(C(C)C)c1CC(=O)N=S(N)(=O)c1cccc(NC(=O)CCc2cn(CCCNC(=O)CCCC3SC[C@@H]4NC(=O)N[C@H]34)nn2)c1] |
| 405b | 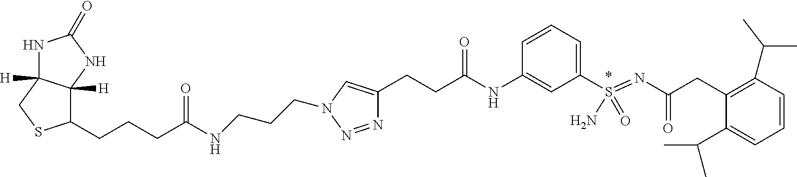 [SMILES: CC(C)c1cccc(C(C)C)c1CC(=O)N=S(N)(=O)c1cccc(NC(=O)CCc2cn(CCCNC(=O)CCCC3SC[C@@H]4NC(=O)N[C@H]34)nn2)c1] |
| 406 | 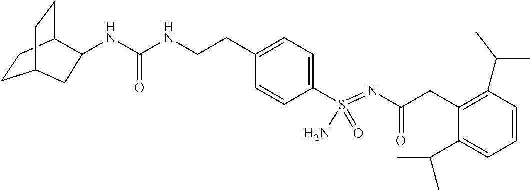 [SMILES: CC(C)c1cccc(C(C)C)c1CC(=O)N=S(N)(=O)c1ccc(CCNC(=O)NC2CC3CCC2CC3)cc1] |
| 406a | 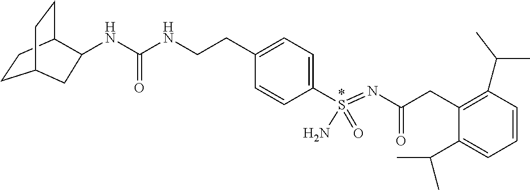 [SMILES: CC(C)c1cccc(C(C)C)c1CC(=O)N=S(N)(=O)c1ccc(CCNC(=O)NC2CC3CCC2CC3)cc1] |
| 406b | 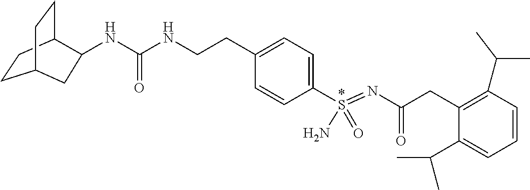 [SMILES: CC(C)c1cccc(C(C)C)c1CC(=O)N=S(N)(=O)c1ccc(CCNC(=O)NC2CC3CCC2CC3)cc1] |
| 407 | 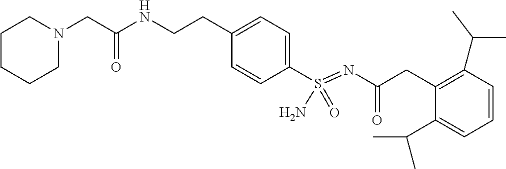 [SMILES: CC(C)c1cccc(C(C)C)c1CC(=O)N=S(N)(=O)c1ccc(CCNC(=O)CN2CCCCC2)cc1] |
| 407a | 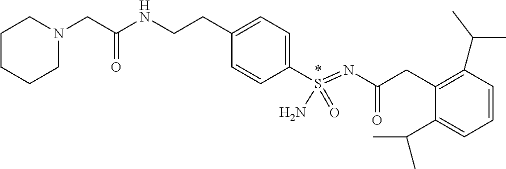 [SMILES: CC(C)c1cccc(C(C)C)c1CC(=O)N=S(N)(=O)c1ccc(CCNC(=O)CN2CCCCC2)cc1] |
| 407b | 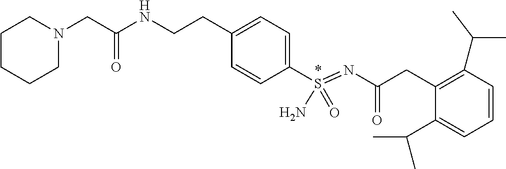 [SMILES: CC(C)c1cccc(C(C)C)c1CC(=O)N=S(N)(=O)c1ccc(CCNC(=O)CN2CCCCC2)cc1] |
| 408 | 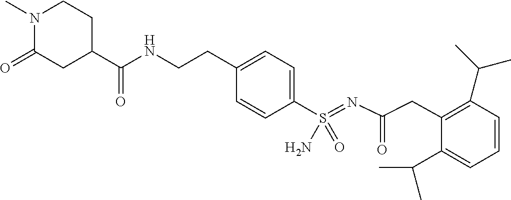 [SMILES: CC(C)c1cccc(C(C)C)c1CC(=O)N=S(N)(=O)c1ccc(CCNC(=O)C2CCN(C)C(=O)C2)cc1] |
| 408a | 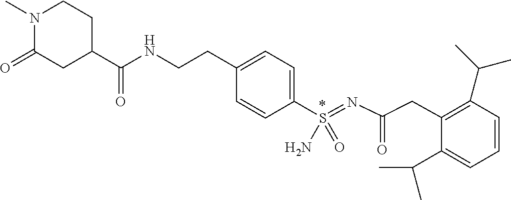 [SMILES: CC(C)c1cccc(C(C)C)c1CC(=O)N=S(N)(=O)c1ccc(CCNC(=O)C2CCN(C)C(=O)C2)cc1] |
| 408b | 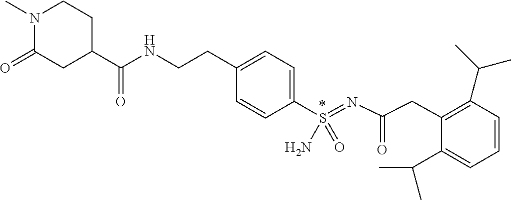 [SMILES: CC(C)c1cccc(C(C)C)c1CC(=O)N=S(N)(=O)c1ccc(CCNC(=O)C2CCN(C)C(=O)C2)cc1] |
| 409 | 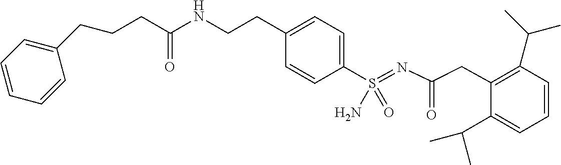 [SMILES: CC(C)c1cccc(C(C)C)c1CC(=O)N=S(N)(=O)c1ccc(CCNC(=O)CCCc2ccccc2)cc1] |
| 409a | 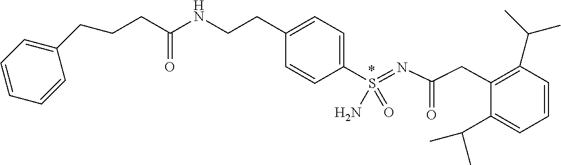 [SMILES: CC(C)c1cccc(C(C)C)c1CC(=O)N=S(N)(=O)c1ccc(CCNC(=O)CCCc2ccccc2)cc1] |
| 409b | 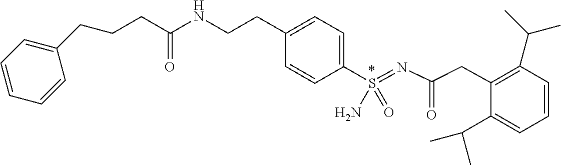 [SMILES: CC(C)c1cccc(C(C)C)c1CC(=O)N=S(N)(=O)c1ccc(CCNC(=O)CCCc2ccccc2)cc1] |
| 410 | 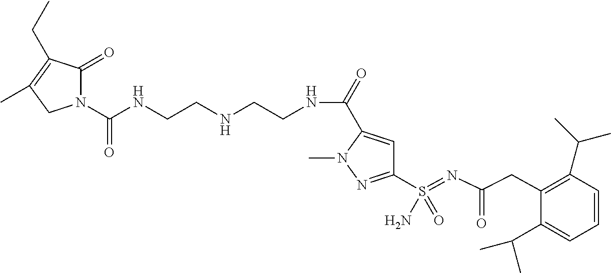 [SMILES: CCC1=C(C)CN(C(=O)NCCNCCNC(=O)c2cc(S(N)(=O)=NC(=O)Cc3c(C(C)C)cccc3C(C)C)nn2C)C1=O] |
| 410a | 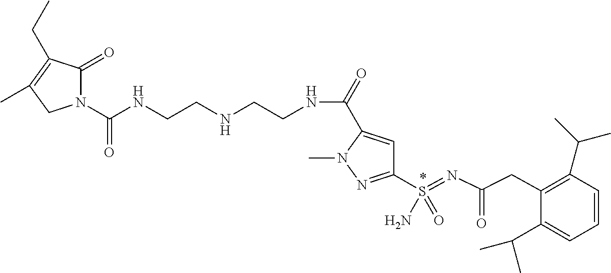 [SMILES: CCC1=C(C)CN(C(=O)NCCNCCNC(=O)c2cc(S(N)(=O)=NC(=O)Cc3c(C(C)C)cccc3C(C)C)nn2C)C1=O] |
| 410b | 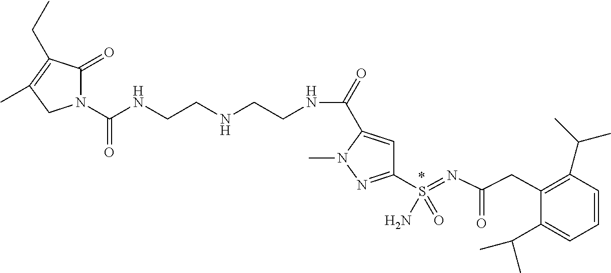 [SMILES: CCC1=C(C)CN(C(=O)NCCNCCNC(=O)c2cc(S(N)(=O)=NC(=O)Cc3c(C(C)C)cccc3C(C)C)nn2C)C1=O] |
| 411 | 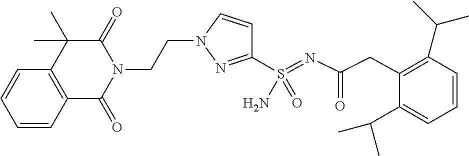 [SMILES: CC(C)c1cccc(C(C)C)c1CC(=O)N=S(N)(=O)c1ccn(CCN2C(=O)c3ccccc3C(C)(C)C2=O)n1] |
| 411a | 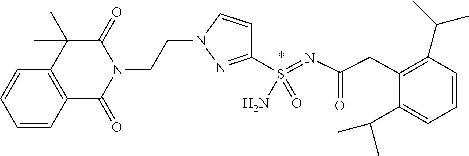 [SMILES: CC(C)c1cccc(C(C)C)c1CC(=O)N=S(N)(=O)c1ccn(CCN2C(=O)c3ccccc3C(C)(C)C2=O)n1] |
| 411b | 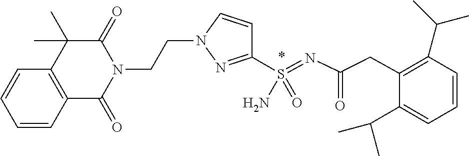 [SMILES: CC(C)c1cccc(C(C)C)c1CC(=O)N=S(N)(=O)c1ccn(CCN2C(=O)c3ccccc3C(C)(C)C2=O)n1] |
| 412 | 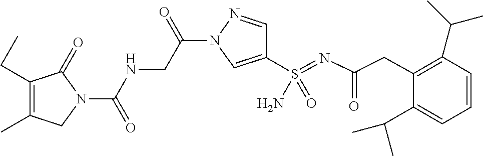 [SMILES: CCC1=C(C)CN(C(=O)NCC(=O)n2cc(S(N)(=O)=NC(=O)Cc3c(C(C)C)cccc3C(C)C)cn2)C1=O] |
| 412a | 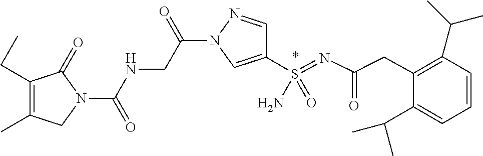 [SMILES: CCC1=C(C)CN(C(=O)NCC(=O)n2cc(S(N)(=O)=NC(=O)Cc3c(C(C)C)cccc3C(C)C)cn2)C1=O] |
| 412b | 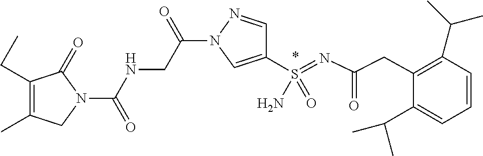 [SMILES: CCC1=C(C)CN(C(=O)NCC(=O)n2cc(S(N)(=O)=NC(=O)Cc3c(C(C)C)cccc3C(C)C)cn2)C1=O] |
| 413 | 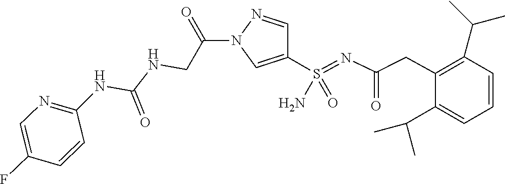 [SMILES: CC(C)c1cccc(C(C)C)c1CC(=O)N=S(N)(=O)c1cnn(C(=O)CNC(=O)Nc2ccc(F)cn2)c1] |
| 413a | 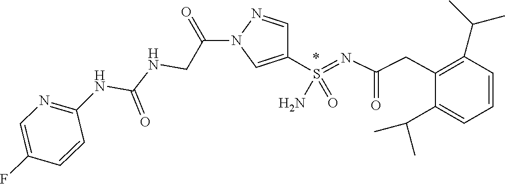 [SMILES: CC(C)c1cccc(C(C)C)c1CC(=O)N=S(N)(=O)c1cnn(C(=O)CNC(=O)Nc2ccc(F)cn2)c1] |
| 413b | 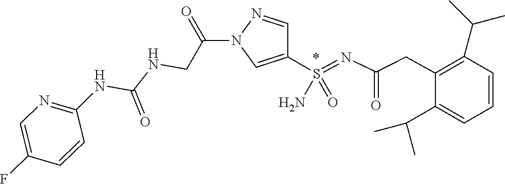 [SMILES: CC(C)c1cccc(C(C)C)c1CC(=O)N=S(N)(=O)c1cnn(C(=O)CNC(=O)Nc2ccc(F)cn2)c1] |
| 414 | 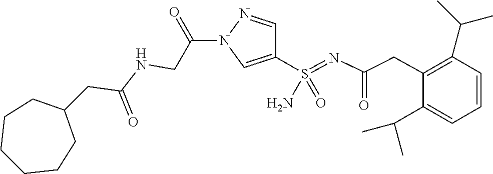 [SMILES: CC(C)c1cccc(C(C)C)c1CC(=O)N=S(N)(=O)c1cnn(C(=O)CNC(=O)CC2CCCCCC2)c1] |
| 414a | 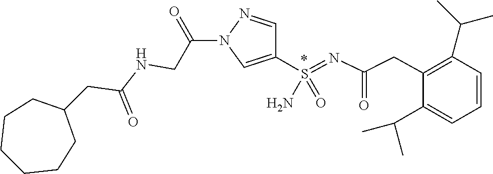 [SMILES: CC(C)c1cccc(C(C)C)c1CC(=O)N=S(N)(=O)c1cnn(C(=O)CNC(=O)CC2CCCCCC2)c1] |
| 414b | 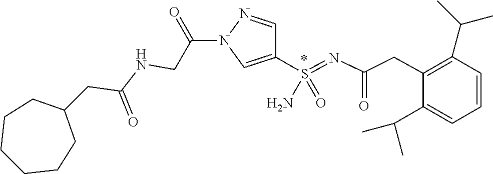 [SMILES: CC(C)c1cccc(C(C)C)c1CC(=O)N=S(N)(=O)c1cnn(C(=O)CNC(=O)CC2CCCCCC2)c1] |
| 415 | 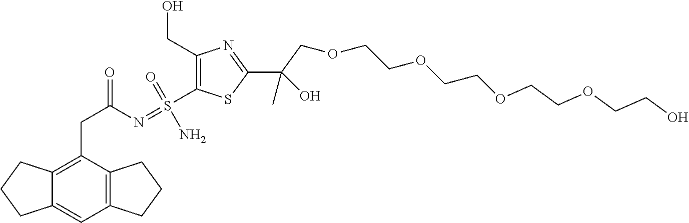 [SMILES: CC(O)(COCCOCCOCCOCCO)c1nc(CO)c(S(N)(=O)=NC(=O)Cc2c3c(cc4c2CCC4)CCC3)s1] |
| 415a | 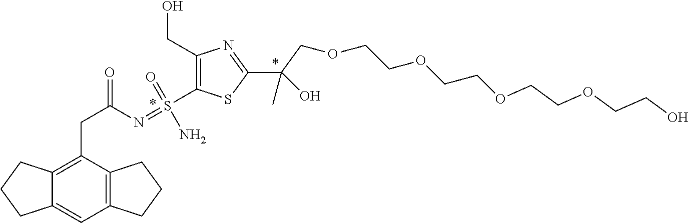 [SMILES: CC(O)(COCCOCCOCCOCCO)c1nc(CO)c(S(N)(=O)=NC(=O)Cc2c3c(cc4c2CCC4)CCC3)s1] |
| 415b | 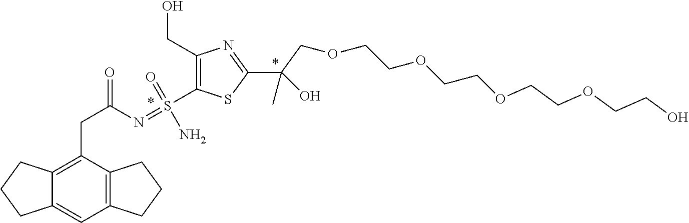 [SMILES: CC(O)(COCCOCCOCCOCCO)c1nc(CO)c(S(N)(=O)=NC(=O)Cc2c3c(cc4c2CCC4)CCC3)s1] |
| 415c | 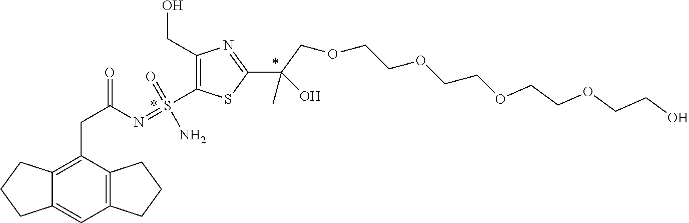 [SMILES: CC(O)(COCCOCCOCCOCCO)c1nc(CO)c(S(N)(=O)=NC(=O)Cc2c3c(cc4c2CCC4)CCC3)s1] |
| 415d | 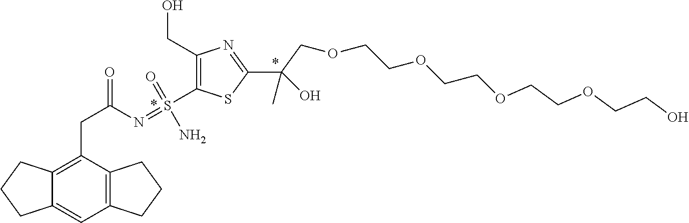 [SMILES: CC(O)(COCCOCCOCCOCCO)c1nc(CO)c(S(N)(=O)=NC(=O)Cc2c3c(cc4c2CCC4)CCC3)s1] |
| 416 | 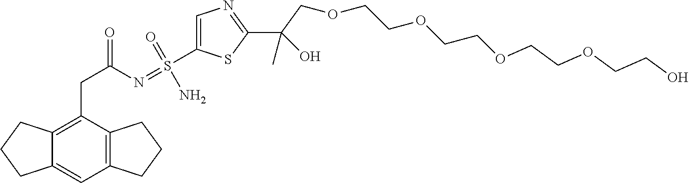 [SMILES: CC(O)(COCCOCCOCCOCCO)c1ncc(S(N)(=O)=NC(=O)Cc2c3c(cc4c2CCC4)CCC3)s1] |
| 416a | 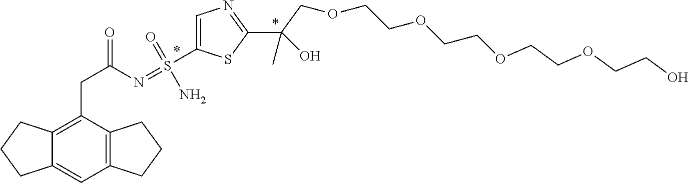 [SMILES: CC(O)(COCCOCCOCCOCCO)c1ncc(S(N)(=O)=NC(=O)Cc2c3c(cc4c2CCC4)CCC3)s1] |
| 416b | 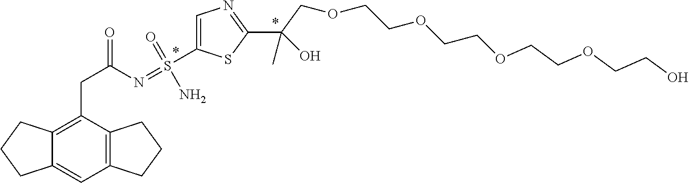 [SMILES: CC(O)(COCCOCCOCCOCCO)c1ncc(S(N)(=O)=NC(=O)Cc2c3c(cc4c2CCC4)CCC3)s1] |
| 416c | 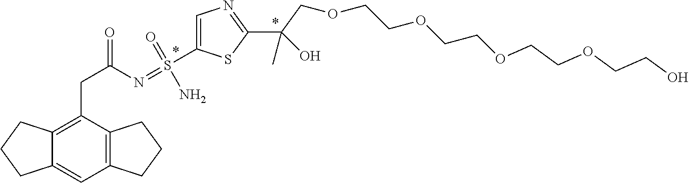 [SMILES: CC(O)(COCCOCCOCCOCCO)c1ncc(S(N)(=O)=NC(=O)Cc2c3c(cc4c2CCC4)CCC3)s1] |
| 416d | 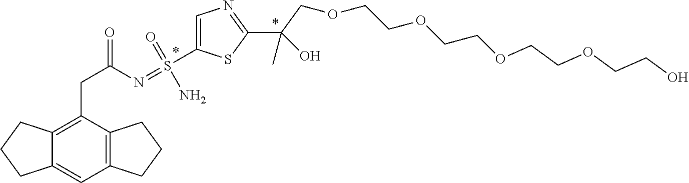 [SMILES: CC(O)(COCCOCCOCCOCCO)c1ncc(S(N)(=O)=NC(=O)Cc2c3c(cc4c2CCC4)CCC3)s1] |
| 417 | 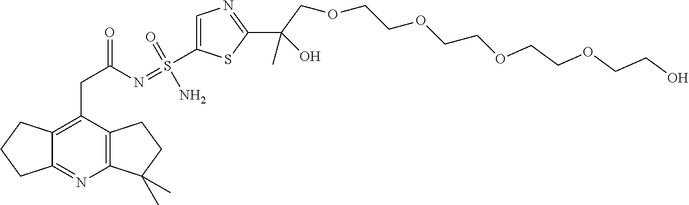 [SMILES: CC1(C)CCc2c1nc1c(c2CC(=O)N=S(N)(=O)c2cnc(C(C)(O)COCCOCCOCCOCCO)s2)CCC1] |
| 417a | 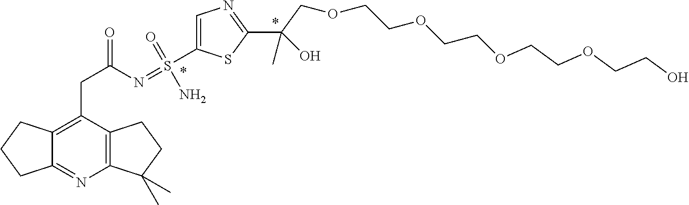 [SMILES: CC1(C)CCc2c1nc1c(c2CC(=O)N=S(N)(=O)c2cnc(C(C)(O)COCCOCCOCCOCCO)s2)CCC1] |
| 417b | 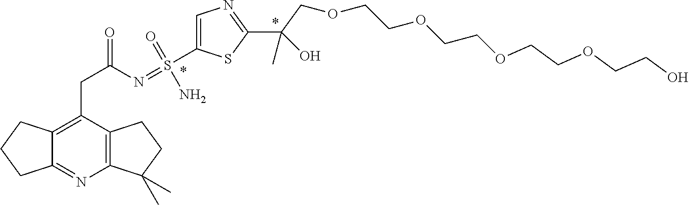 [SMILES: CC1(C)CCc2c1nc1c(c2CC(=O)N=S(N)(=O)c2cnc(C(C)(O)COCCOCCOCCOCCO)s2)CCC1] |
| 417c | 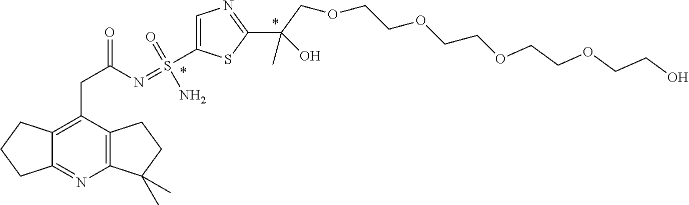 [SMILES: CC1(C)CCc2c1nc1c(c2CC(=O)N=S(N)(=O)c2cnc(C(C)(O)COCCOCCOCCOCCO)s2)CCC1] |
| 417d | 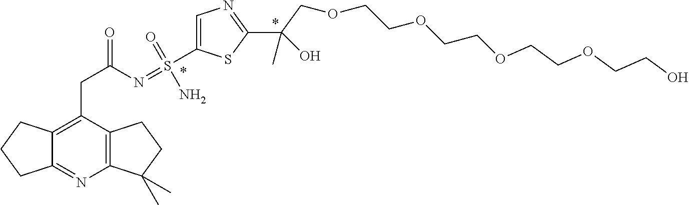 [SMILES: CC1(C)CCc2c1nc1c(c2CC(=O)N=S(N)(=O)c2cnc(C(C)(O)COCCOCCOCCOCCO)s2)CCC1] |
| 431b | 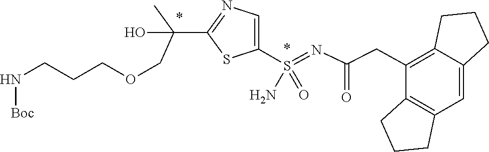 [SMILES: CC(C)(C)OC(=O)NCCCOCC(C)(O)c1ncc(S(N)(=O)=NC(=O)Cc2c3c(cc4c2CCC4)CCC3)s1] |
| 431c | 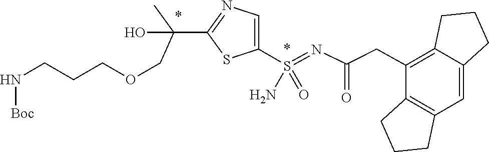 [SMILES: CC(C)(C)OC(=O)NCCCOCC(C)(O)c1ncc(S(N)(=O)=NC(=O)Cc2c3c(cc4c2CCC4)CCC3)s1] |
| 431d | 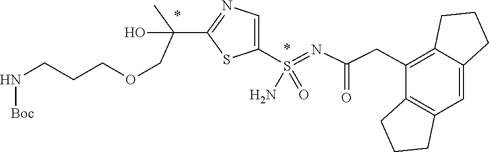 [SMILES: CC(C)(C)OC(=O)NCCCOCC(C)(O)c1ncc(S(N)(=O)=NC(=O)Cc2c3c(cc4c2CCC4)CCC3)s1] |
| 432 | 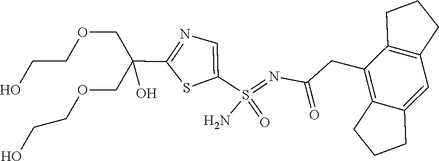 [SMILES: NS(=O)(=NC(=O)Cc1c2c(cc3c1CCC3)CCC2)c1cnc(C(O)(COCCO)COCCO)s1] |
| 432a | 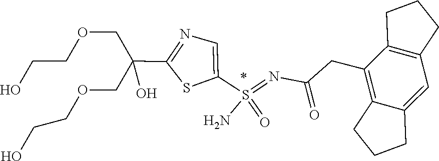 [SMILES: NS(=O)(=NC(=O)Cc1c2c(cc3c1CCC3)CCC2)c1cnc(C(O)(COCCO)COCCO)s1] |
| 432b | 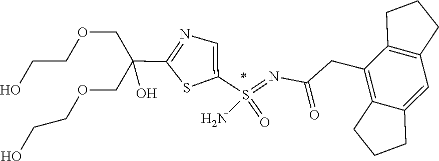 [SMILES: NS(=O)(=NC(=O)Cc1c2c(cc3c1CCC3)CCC2)c1cnc(C(O)(COCCO)COCCO)s1] |
| 433 | 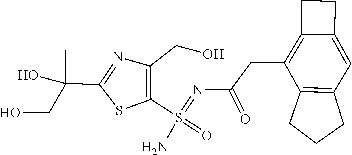 [SMILES: CC(O)(CO)c1nc(CO)c(S(N)(=O)=NC(=O)Cc2c3c(cc4c2CC4)CCC3)s1] |
| 433a | 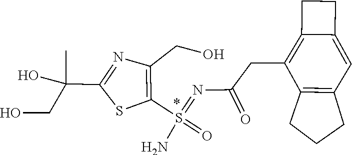 [SMILES: CC(O)(CO)c1nc(CO)c(S(N)(=O)=NC(=O)Cc2c3c(cc4c2CC4)CCC3)s1] |
| 433b | 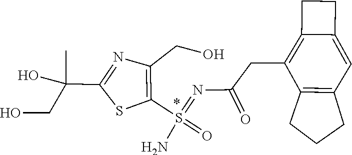 [SMILES: CC(O)(CO)c1nc(CO)c(S(N)(=O)=NC(=O)Cc2c3c(cc4c2CC4)CCC3)s1] |
| 435 | 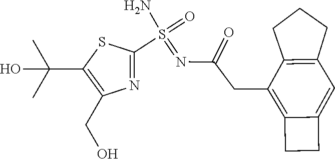 [SMILES: CC(C)(O)c1sc(S(N)(=O)=NC(=O)Cc2c3c(cc4c2CC4)CCC3)nc1CO] |
| 435a | 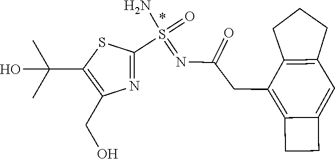 [SMILES: CC(C)(O)c1sc(S(N)(=O)=NC(=O)Cc2c3c(cc4c2CC4)CCC3)nc1CO] |
| 435b | 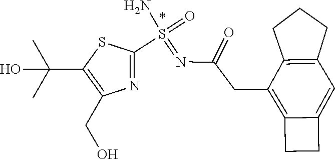 [SMILES: CC(C)(O)c1sc(S(N)(=O)=NC(=O)Cc2c3c(cc4c2CC4)CCC3)nc1CO] |
| 436 | 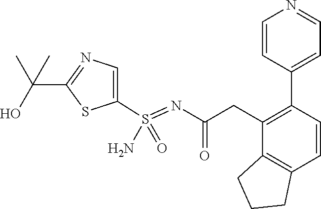 [SMILES: CC(C)(O)c1ncc(S(N)(=O)=NC(=O)Cc2c(-c3ccncc3)ccc3c2CCC3)s1] |
| 436a | 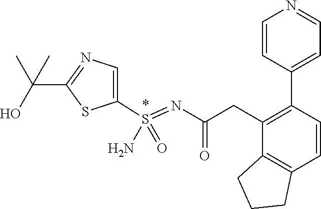 [SMILES: CC(C)(O)c1ncc(S(N)(=O)=NC(=O)Cc2c(-c3ccncc3)ccc3c2CCC3)s1] |
| 436b | 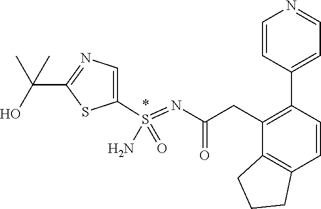 [SMILES: CC(C)(O)c1ncc(S(N)(=O)=NC(=O)Cc2c(-c3ccncc3)ccc3c2CCC3)s1] |
| 437 | 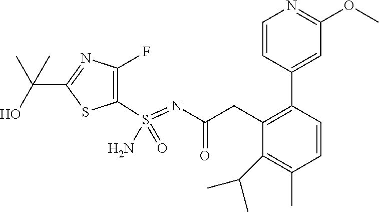 [SMILES: COc1cc(-c2ccc(C)c(C(C)C)c2CC(=O)N=S(N)(=O)c2sc(C(C)(C)O)nc2F)ccn1] |
| 437a | 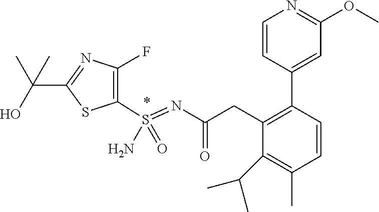 [SMILES: COc1cc(-c2ccc(C)c(C(C)C)c2CC(=O)N=S(N)(=O)c2sc(C(C)(C)O)nc2F)ccn1] |
| 437b | 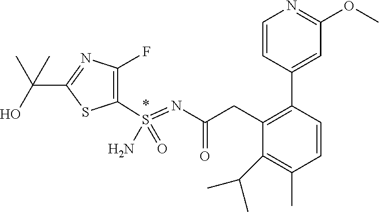 [SMILES: COc1cc(-c2ccc(C)c(C(C)C)c2CC(=O)N=S(N)(=O)c2sc(C(C)(C)O)nc2F)ccn1] |
| 438 | 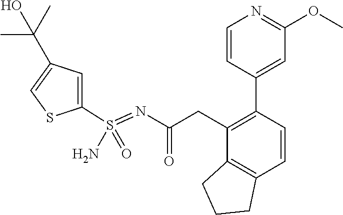 [SMILES: COc1cc(-c2ccc3c(c2CC(=O)N=S(N)(=O)c2cc(C(C)(C)O)cs2)CCC3)ccn1] |
| 438a | 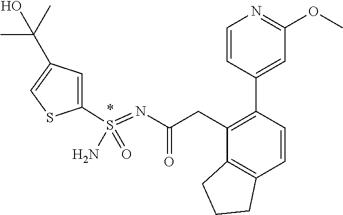 [SMILES: COc1cc(-c2ccc3c(c2CC(=O)N=S(N)(=O)c2cc(C(C)(C)O)cs2)CCC3)ccn1] |
| 438b | 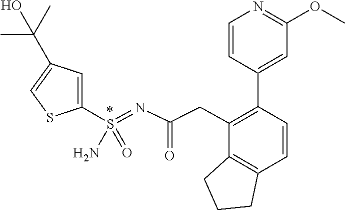 [SMILES: COc1cc(-c2ccc3c(c2CC(=O)N=S(N)(=O)c2cc(C(C)(C)O)cs2)CCC3)ccn1] |
| 439 | 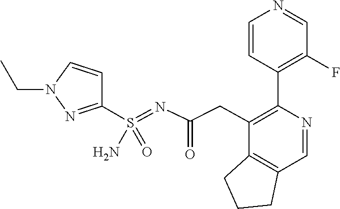 [SMILES: CCn1ccc(S(N)(=O)=NC(=O)Cc2c(-c3ccncc3F)ncc3c2CCC3)n1] |
| 439a | 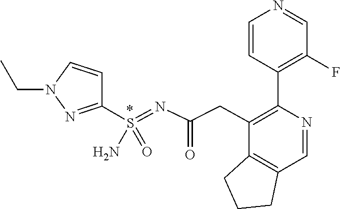 [SMILES: CCn1ccc(S(N)(=O)=NC(=O)Cc2c(-c3ccncc3F)ncc3c2CCC3)n1] |
| 439b | 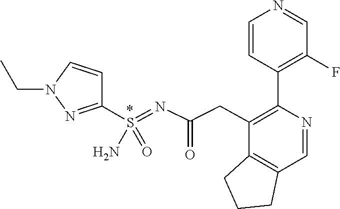 [SMILES: CCn1ccc(S(N)(=O)=NC(=O)Cc2c(-c3ccncc3F)ncc3c2CCC3)n1] |
| 440 | 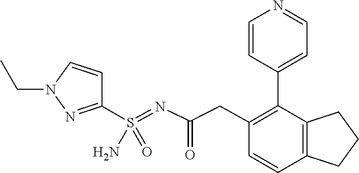 [SMILES: CCn1ccc(S(N)(=O)=NC(=O)Cc2ccc3c(c2-c2ccncc2)CCC3)n1] |
| 440a | 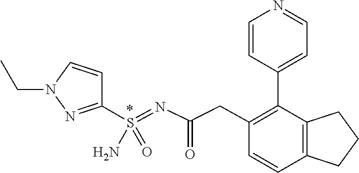 [SMILES: CCn1ccc(S(N)(=O)=NC(=O)Cc2ccc3c(c2-c2ccncc2)CCC3)n1] |
| 440b | 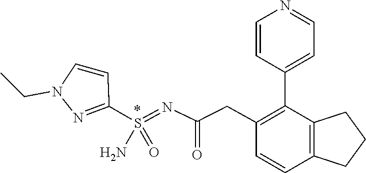 [SMILES: CCn1ccc(S(N)(=O)=NC(=O)Cc2ccc3c(c2-c2ccncc2)CCC3)n1] |
| 441 | 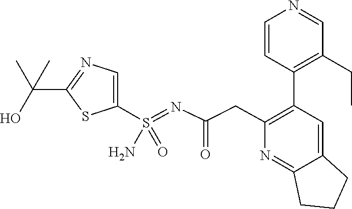 [SMILES: CCc1cnccc1-c1cc2c(nc1CC(=O)N=S(N)(=O)c1cnc(C(C)(C)O)s1)CCC2] |
| 441a | 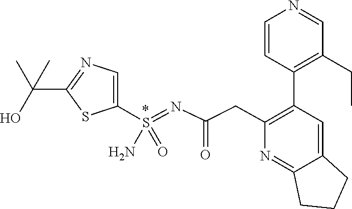 [SMILES: CCc1cnccc1-c1cc2c(nc1CC(=O)N=S(N)(=O)c1cnc(C(C)(C)O)s1)CCC2] |
| 441b | 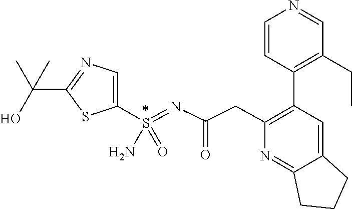 [SMILES: CCc1cnccc1-c1cc2c(nc1CC(=O)N=S(N)(=O)c1cnc(C(C)(C)O)s1)CCC2] |
| 442 | 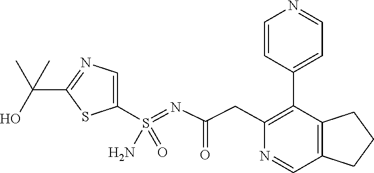 [SMILES: CC(C)(O)c1ncc(S(N)(=O)=NC(=O)Cc2ncc3c(c2-c2ccncc2)CCC3)s1] |
| 442a | 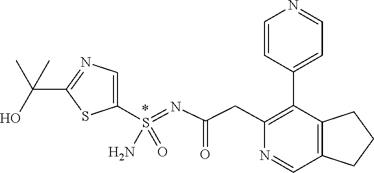 [SMILES: CC(C)(O)c1ncc(S(N)(=O)=NC(=O)Cc2ncc3c(c2-c2ccncc2)CCC3)s1] |
| 442b | 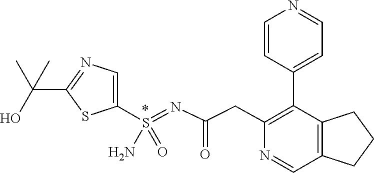 [SMILES: CC(C)(O)c1ncc(S(N)(=O)=NC(=O)Cc2ncc3c(c2-c2ccncc2)CCC3)s1] |
| 443 | 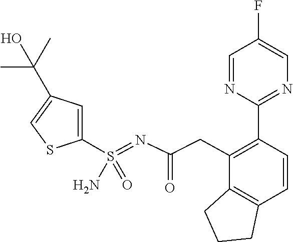 [SMILES: CC(C)(O)c1csc(S(N)(=O)=NC(=O)Cc2c(-c3ncc(F)cn3)ccc3c2CCC3)c1] |
| 443a | 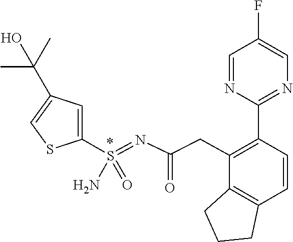 [SMILES: CC(C)(O)c1csc(S(N)(=O)=NC(=O)Cc2c(-c3ncc(F)cn3)ccc3c2CCC3)c1] |
| 443b | 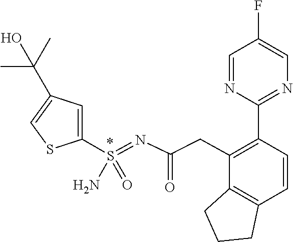 [SMILES: CC(C)(O)c1csc(S(N)(=O)=NC(=O)Cc2c(-c3ncc(F)cn3)ccc3c2CCC3)c1] |
| 444 | 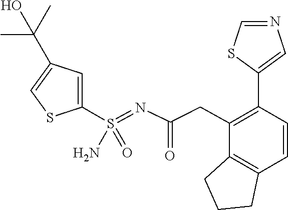 [SMILES: CC(C)(O)c1csc(S(N)(=O)=NC(=O)Cc2c(-c3cncs3)ccc3c2CCC3)c1] |
| 444a | 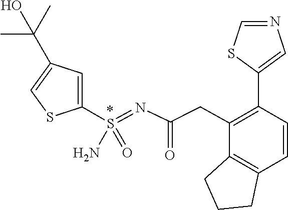 [SMILES: CC(C)(O)c1csc(S(N)(=O)=NC(=O)Cc2c(-c3cncs3)ccc3c2CCC3)c1] |
| 444b | 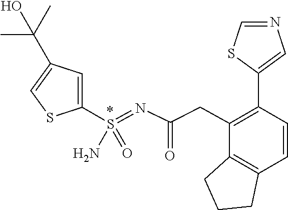 [SMILES: CC(C)(O)c1csc(S(N)(=O)=NC(=O)Cc2c(-c3cncs3)ccc3c2CCC3)c1] |
| 445 | 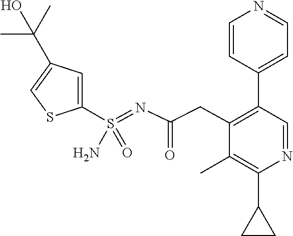 [SMILES: Cc1c(C2CC2)ncc(-c2ccncc2)c1CC(=O)N=S(N)(=O)c1cc(C(C)(C)O)cs1] |
| 445a | 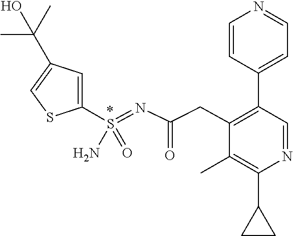 [SMILES: Cc1c(C2CC2)ncc(-c2ccncc2)c1CC(=O)N=S(N)(=O)c1cc(C(C)(C)O)cs1] |
| 445b | 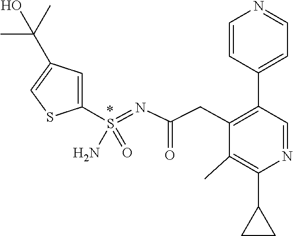 [SMILES: Cc1c(C2CC2)ncc(-c2ccncc2)c1CC(=O)N=S(N)(=O)c1cc(C(C)(C)O)cs1] |
| 446 | 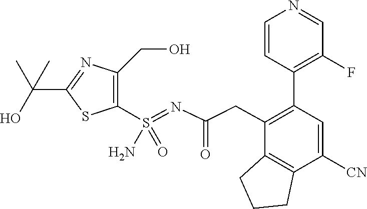 [SMILES: CC(C)(O)c1nc(CO)c(S(N)(=O)=NC(=O)Cc2c(-c3ccncc3F)cc(C#N)c3c2CCC3)s1] |
| 446a | 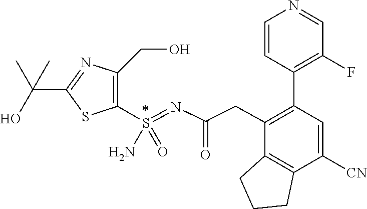 [SMILES: CC(C)(O)c1nc(CO)c(S(N)(=O)=NC(=O)Cc2c(-c3ccncc3F)cc(C#N)c3c2CCC3)s1] |
| 446b | 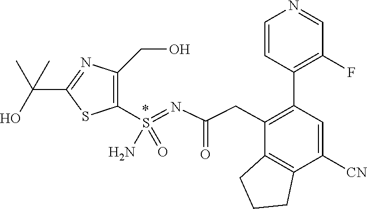 [SMILES: CC(C)(O)c1nc(CO)c(S(N)(=O)=NC(=O)Cc2c(-c3ccncc3F)cc(C#N)c3c2CCC3)s1] |
| 447 | 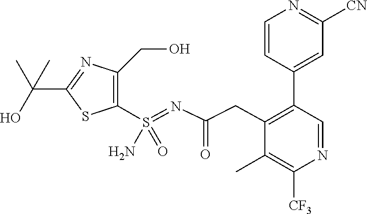 [SMILES: Cc1c(C(F)(F)F)ncc(-c2ccnc(C#N)c2)c1CC(=O)N=S(N)(=O)c1sc(C(C)(C)O)nc1CO] |
| 447a | 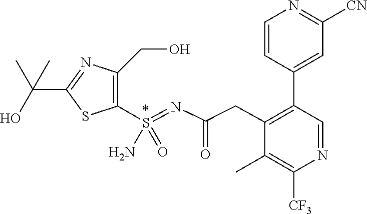 [SMILES: Cc1c(C(F)(F)F)ncc(-c2ccnc(C#N)c2)c1CC(=O)N=S(N)(=O)c1sc(C(C)(C)O)nc1CO] |
| 447b | 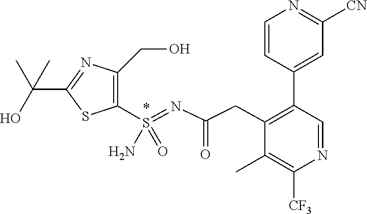 [SMILES: Cc1c(C(F)(F)F)ncc(-c2ccnc(C#N)c2)c1CC(=O)N=S(N)(=O)c1sc(C(C)(C)O)nc1CO] |
| 448 | 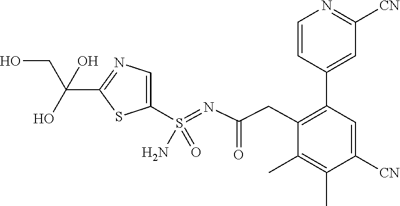 [SMILES: Cc1c(C#N)cc(-c2ccnc(C#N)c2)c(CC(=O)N=S(N)(=O)c2cnc(C(O)(O)CO)s2)c1C] |
| 448a | 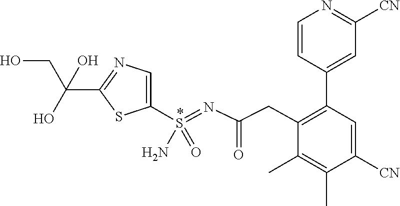 [SMILES: Cc1c(C#N)cc(-c2ccnc(C#N)c2)c(CC(=O)N=S(N)(=O)c2cnc(C(O)(O)CO)s2)c1C] |
| 448b | 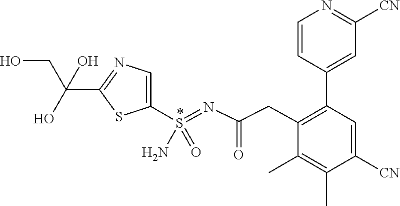 [SMILES: Cc1c(C#N)cc(-c2ccnc(C#N)c2)c(CC(=O)N=S(N)(=O)c2cnc(C(O)(O)CO)s2)c1C] |
| 449 | 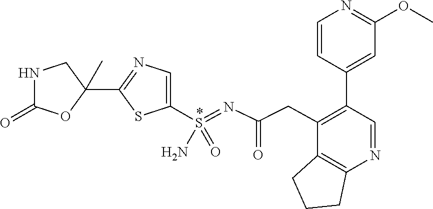 [SMILES: COc1cc(-c2cnc3c(c2CC(=O)N=S(N)(=O)c2cnc(C4(C)CNC(=O)O4)s2)CCC3)ccn1] |
| 449a | 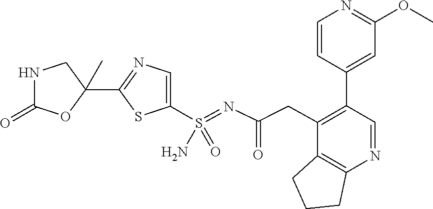 [SMILES: COc1cc(-c2cnc3c(c2CC(=O)N=S(N)(=O)c2cnc(C4(C)CNC(=O)O4)s2)CCC3)ccn1] |
| 449b | 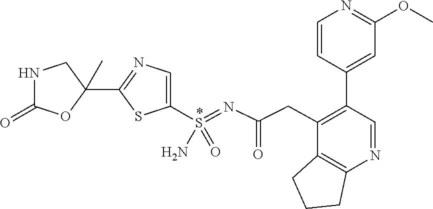 [SMILES: COc1cc(-c2cnc3c(c2CC(=O)N=S(N)(=O)c2cnc(C4(C)CNC(=O)O4)s2)CCC3)ccn1] |
| 450 | 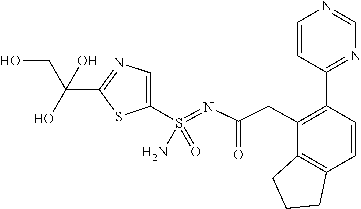 [SMILES: NS(=O)(=NC(=O)Cc1c(-c2ccncn2)ccc2c1CCC2)c1cnc(C(O)(O)CO)s1] |
| 450a | 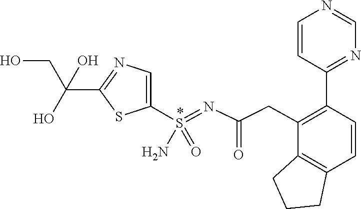 [SMILES: NS(=O)(=NC(=O)Cc1c(-c2ccncn2)ccc2c1CCC2)c1cnc(C(O)(O)CO)s1] |
| 418 | 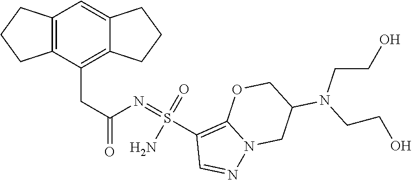 [SMILES: NS(=O)(=NC(=O)Cc1c2c(cc3c1CCC3)CCC2)c1cnn2c1OCC(N(CCO)CCO)C2] |
| 450b | 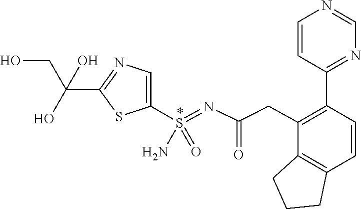 [SMILES: NS(=O)(=NC(=O)Cc1c(-c2ccncn2)ccc2c1CCC2)c1cnc(C(O)(O)CO)s1] |
| 418a | 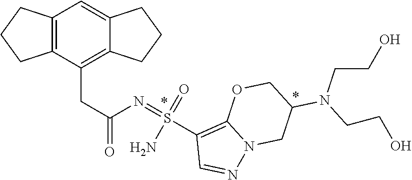 [SMILES: NS(=O)(=NC(=O)Cc1c2c(cc3c1CCC3)CCC2)c1cnn2c1OCC(N(CCO)CCO)C2] |
| 451 | 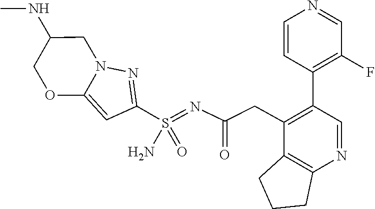 [SMILES: CNC1COc2cc(S(N)(=O)=NC(=O)Cc3c(-c4ccncc4F)cnc4c3CCC4)nn2C1] |
| 431b | 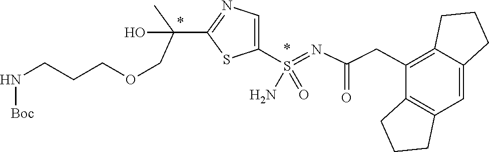 [SMILES: CC(C)(C)OC(=O)NCCCOCC(C)(O)c1ncc(S(N)(=O)=NC(=O)Cc2c3c(cc4c2CCC4)CCC3)s1] |
| 418b | 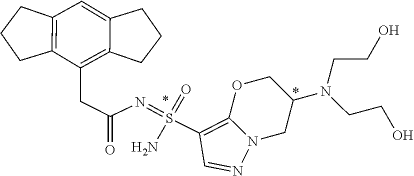 [SMILES: NS(=O)(=NC(=O)Cc1c2c(cc3c1CCC3)CCC2)c1cnn2c1OCC(N(CCO)CCO)C2] |
| 418c | 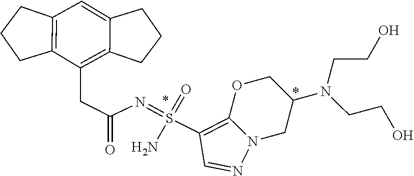 [SMILES: NS(=O)(=NC(=O)Cc1c2c(cc3c1CCC3)CCC2)c1cnn2c1OCC(N(CCO)CCO)C2] |
| 418d | 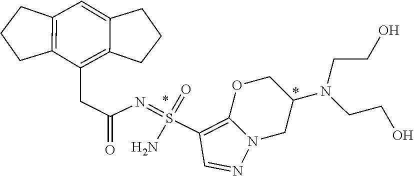 [SMILES: NS(=O)(=NC(=O)Cc1c2c(cc3c1CCC3)CCC2)c1cnn2c1OCC(N(CCO)CCO)C2] |
| 419 | 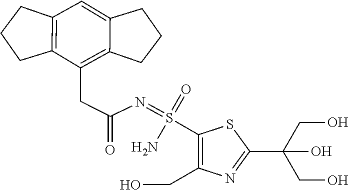 [SMILES: NS(=O)(=NC(=O)Cc1c2c(cc3c1CCC3)CCC2)c1sc(C(O)(CO)CO)nc1CO] |
| 419a | 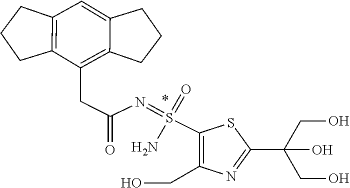 [SMILES: NS(=O)(=NC(=O)Cc1c2c(cc3c1CCC3)CCC2)c1sc(C(O)(CO)CO)nc1CO] |
| 419b | 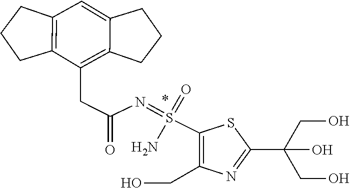 [SMILES: NS(=O)(=NC(=O)Cc1c2c(cc3c1CCC3)CCC2)c1sc(C(O)(CO)CO)nc1CO] |
| 420 | 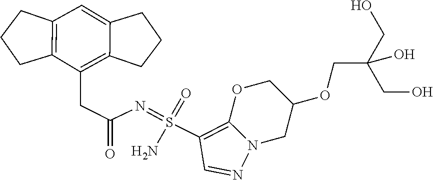 [SMILES: NS(=O)(=NC(=O)Cc1c2c(cc3c1CCC3)CCC2)c1cnn2c1OCC(OCC(O)(CO)CO)C2] |
| 420a | 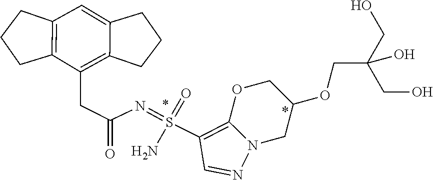 [SMILES: NS(=O)(=NC(=O)Cc1c2c(cc3c1CCC3)CCC2)c1cnn2c1OCC(OCC(O)(CO)CO)C2] |
| 420b | 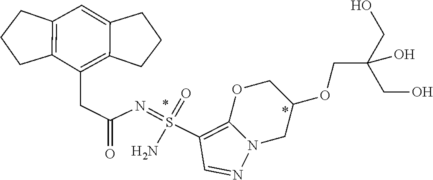 [SMILES: NS(=O)(=NC(=O)Cc1c2c(cc3c1CCC3)CCC2)c1cnn2c1OCC(OCC(O)(CO)CO)C2] |
| 420c | 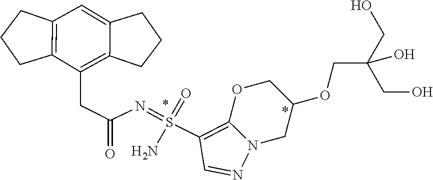 [SMILES: NS(=O)(=NC(=O)Cc1c2c(cc3c1CCC3)CCC2)c1cnn2c1OCC(OCC(O)(CO)CO)C2] |
| 420d | 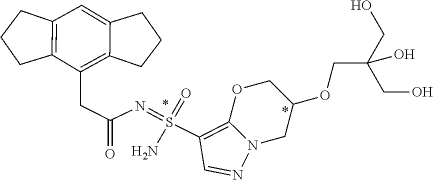 [SMILES: NS(=O)(=NC(=O)Cc1c2c(cc3c1CCC3)CCC2)c1cnn2c1OCC(OCC(O)(CO)CO)C2] |
| 421 | 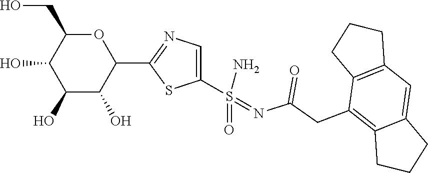 [SMILES: NS(=O)(=NC(=O)Cc1c2c(cc3c1CCC3)CCC2)c1cnc(C2O[C@H](CO)[C@@H](O)[C@H](O)[C@H]2O)s1] |
| 421a | 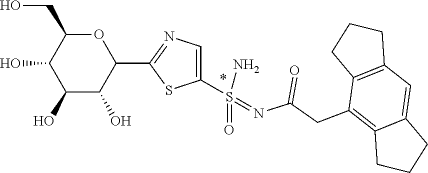 [SMILES: NS(=O)(=NC(=O)Cc1c2c(cc3c1CCC3)CCC2)c1cnc(C2O[C@H](CO)[C@@H](O)[C@H](O)[C@H]2O)s1] |
| 421b | 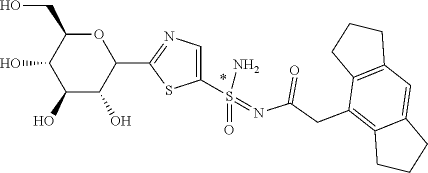 [SMILES: NS(=O)(=NC(=O)Cc1c2c(cc3c1CCC3)CCC2)c1cnc(C2O[C@H](CO)[C@@H](O)[C@H](O)[C@H]2O)s1] |
| 422 | 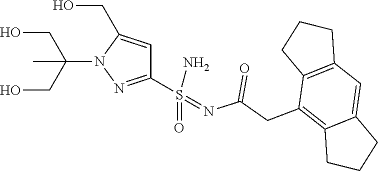 [SMILES: CC(CO)(CO)n1nc(S(N)(=O)=NC(=O)Cc2c3c(cc4c2CCC4)CCC3)cc1CO] |
| 422a | 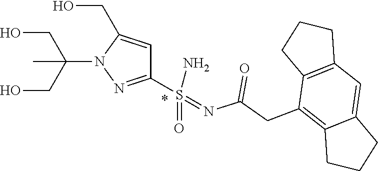 [SMILES: CC(CO)(CO)n1nc(S(N)(=O)=NC(=O)Cc2c3c(cc4c2CCC4)CCC3)cc1CO] |
| 422b | 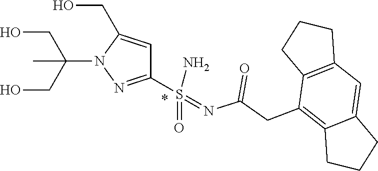 [SMILES: CC(CO)(CO)n1nc(S(N)(=O)=NC(=O)Cc2c3c(cc4c2CCC4)CCC3)cc1CO] |
| 423 | 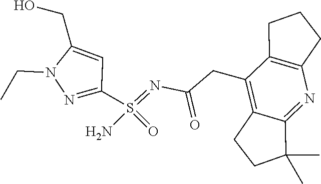 [SMILES: CCn1nc(S(N)(=O)=NC(=O)Cc2c3c(nc4c2CCC4(C)C)CCC3)cc1CO] |
| 423a | 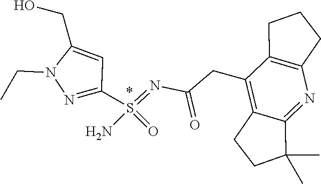 [SMILES: CCn1nc(S(N)(=O)=NC(=O)Cc2c3c(nc4c2CCC4(C)C)CCC3)cc1CO] |
| 423b | 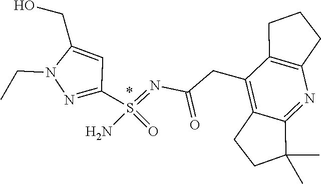 [SMILES: CCn1nc(S(N)(=O)=NC(=O)Cc2c3c(nc4c2CCC4(C)C)CCC3)cc1CO] |
| 424 | 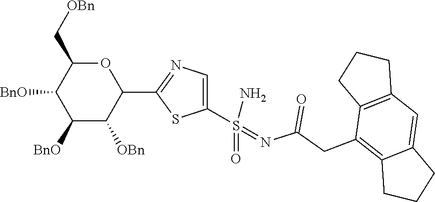 [SMILES: NS(=O)(=NC(=O)Cc1c2c(cc3c1CCC3)CCC2)c1cnc(C2O[C@H](COCc3ccccc3)[C@@H](OCc3ccccc3)[C@H](OCc3ccccc3)[C@H]2OCc2ccccc2)s1] |
| 424a | 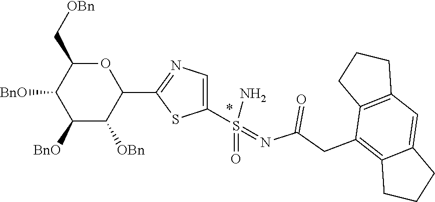 [SMILES: NS(=O)(=NC(=O)Cc1c2c(cc3c1CCC3)CCC2)c1cnc(C2O[C@H](COCc3ccccc3)[C@@H](OCc3ccccc3)[C@H](OCc3ccccc3)[C@H]2OCc2ccccc2)s1] |
| 424b | 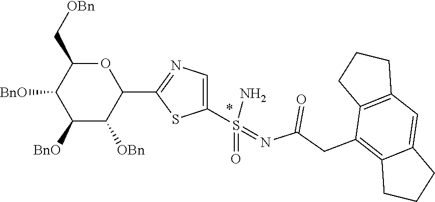 [SMILES: NS(=O)(=NC(=O)Cc1c2c(cc3c1CCC3)CCC2)c1cnc(C2O[C@H](COCc3ccccc3)[C@@H](OCc3ccccc3)[C@H](OCc3ccccc3)[C@H]2OCc2ccccc2)s1] |
| 425 | 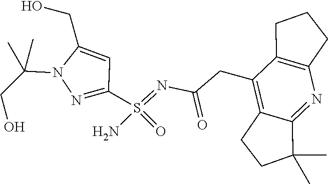 [SMILES: CC1(C)CCc2c1nc1c(c2CC(=O)N=S(N)(=O)c2cc(CO)n(C(C)(C)CO)n2)CCC1] |
| 425a | 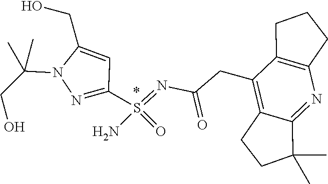 [SMILES: CC1(C)CCc2c1nc1c(c2CC(=O)N=S(N)(=O)c2cc(CO)n(C(C)(C)CO)n2)CCC1] |
| 425b | 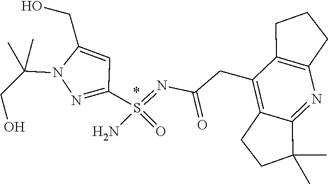 [SMILES: CC1(C)CCc2c1nc1c(c2CC(=O)N=S(N)(=O)c2cc(CO)n(C(C)(C)CO)n2)CCC1] |
| 426 | 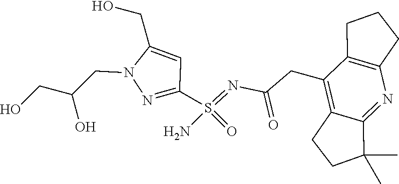 [SMILES: CC1(C)CCc2c1nc1c(c2CC(=O)N=S(N)(=O)c2cc(CO)n(CC(O)CO)n2)CCC1] |
| 426a | 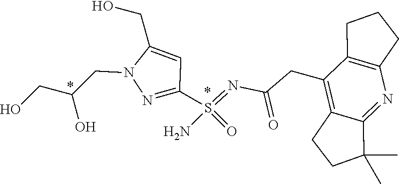 [SMILES: CC1(C)CCc2c1nc1c(c2CC(=O)N=S(N)(=O)c2cc(CO)n(CC(O)CO)n2)CCC1] |
| 426b | 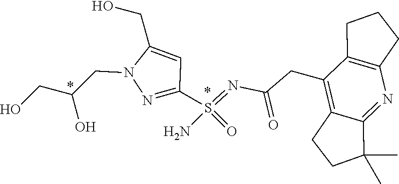 [SMILES: CC1(C)CCc2c1nc1c(c2CC(=O)N=S(N)(=O)c2cc(CO)n(CC(O)CO)n2)CCC1] |
| 426c | 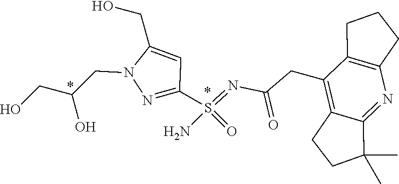 [SMILES: CC1(C)CCc2c1nc1c(c2CC(=O)N=S(N)(=O)c2cc(CO)n(CC(O)CO)n2)CCC1] |
| 426d | 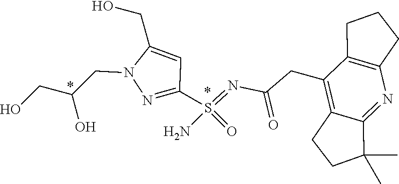 [SMILES: CC1(C)CCc2c1nc1c(c2CC(=O)N=S(N)(=O)c2cc(CO)n(CC(O)CO)n2)CCC1] |
| 427 | 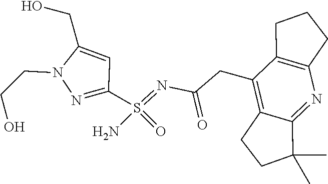 [SMILES: CC1(C)CCc2c1nc1c(c2CC(=O)N=S(N)(=O)c2cc(CO)n(CCO)n2)CCC1] |
| 427a | 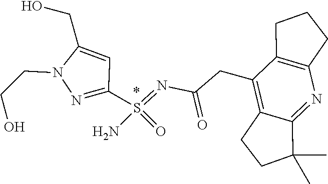 [SMILES: CC1(C)CCc2c1nc1c(c2CC(=O)N=S(N)(=O)c2cc(CO)n(CCO)n2)CCC1] |
| 427b | 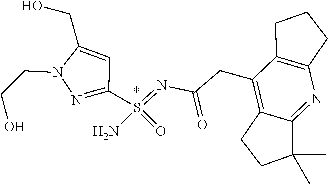 [SMILES: CC1(C)CCc2c1nc1c(c2CC(=O)N=S(N)(=O)c2cc(CO)n(CCO)n2)CCC1] |
| 428 | 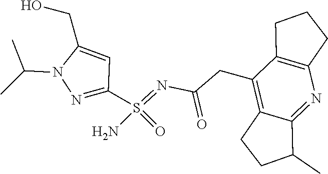 [SMILES: CC1CCc2c1nc1c(c2CC(=O)N=S(N)(=O)c2cc(CO)n(C(C)C)n2)CCC1] |
| 428a | 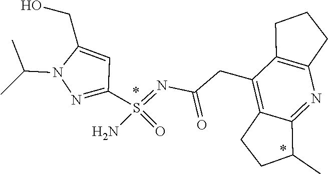 [SMILES: CC1CCc2c1nc1c(c2CC(=O)N=S(N)(=O)c2cc(CO)n(C(C)C)n2)CCC1] |
| 428b | 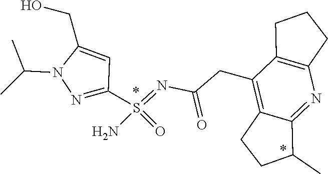 [SMILES: CC1CCc2c1nc1c(c2CC(=O)N=S(N)(=O)c2cc(CO)n(C(C)C)n2)CCC1] |
| 428c | 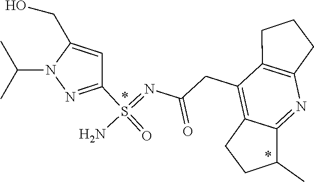 [SMILES: CC1CCc2c1nc1c(c2CC(=O)N=S(N)(=O)c2cc(CO)n(C(C)C)n2)CCC1] |
| 428d | 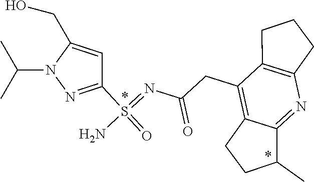 [SMILES: CC1CCc2c1nc1c(c2CC(=O)N=S(N)(=O)c2cc(CO)n(C(C)C)n2)CCC1] |
| 429 | 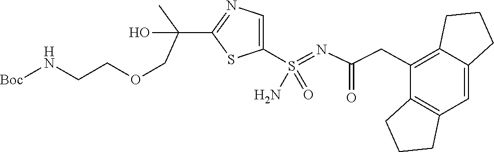 [SMILES: CC(C)(C)OC(=O)NCCOCC(C)(O)c1ncc(S(N)(=O)=NC(=O)Cc2c3c(cc4c2CCC4)CCC3)s1] |
| 429a | 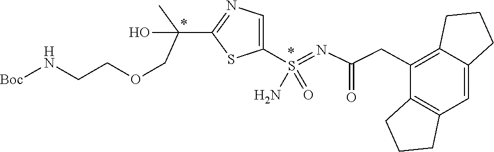 [SMILES: CC(C)(C)OC(=O)NCCOCC(C)(O)c1ncc(S(N)(=O)=NC(=O)Cc2c3c(cc4c2CCC4)CCC3)s1] |
| 429b | 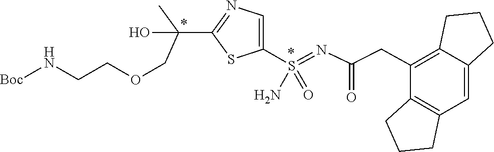 [SMILES: CC(C)(C)OC(=O)NCCOCC(C)(O)c1ncc(S(N)(=O)=NC(=O)Cc2c3c(cc4c2CCC4)CCC3)s1] |
| 429c | 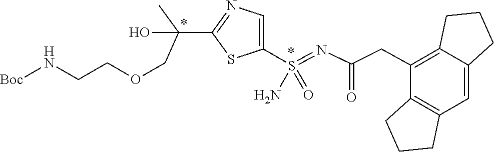 [SMILES: CC(C)(C)OC(=O)NCCOCC(C)(O)c1ncc(S(N)(=O)=NC(=O)Cc2c3c(cc4c2CCC4)CCC3)s1] |
| 429d | 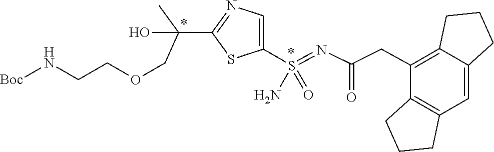 [SMILES: CC(C)(C)OC(=O)NCCOCC(C)(O)c1ncc(S(N)(=O)=NC(=O)Cc2c3c(cc4c2CCC4)CCC3)s1] |
| 430 | 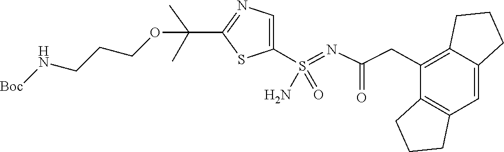 [SMILES: CC(C)(C)OC(=O)NCCCOC(C)(C)c1ncc(S(N)(=O)=NC(=O)Cc2c3c(cc4c2CCC4)CCC3)s1] |
| 430a | 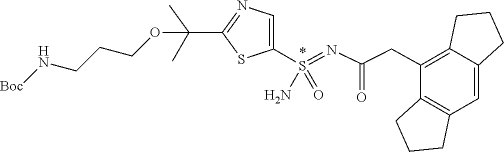 [SMILES: CC(C)(C)OC(=O)NCCCOC(C)(C)c1ncc(S(N)(=O)=NC(=O)Cc2c3c(cc4c2CCC4)CCC3)s1] |
| 430b | 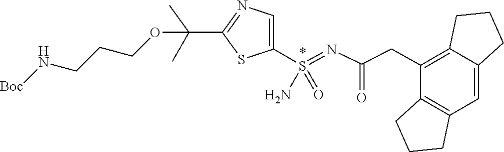 [SMILES: CC(C)(C)OC(=O)NCCCOC(C)(C)c1ncc(S(N)(=O)=NC(=O)Cc2c3c(cc4c2CCC4)CCC3)s1] |
| 431 | 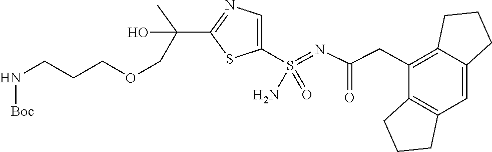 [SMILES: CC(C)(C)OC(=O)NCCCOCC(C)(O)c1ncc(S(N)(=O)=NC(=O)Cc2c3c(cc4c2CCC4)CCC3)s1] |
| 431a | 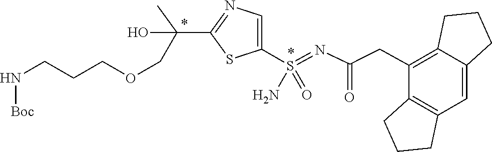 [SMILES: CC(C)(C)OC(=O)NCCCOCC(C)(O)c1ncc(S(N)(=O)=NC(=O)Cc2c3c(cc4c2CCC4)CCC3)s1] |
| 451a | 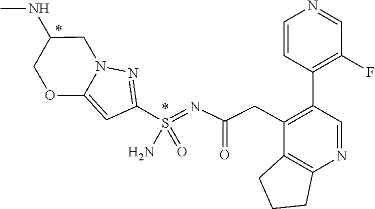 [SMILES: CNC1COc2cc(S(N)(=O)=NC(=O)Cc3c(-c4ccncc4F)cnc4c3CCC4)nn2C1] |
| 451b | 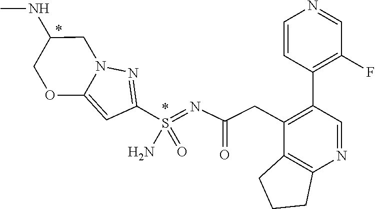 [SMILES: CNC1COc2cc(S(N)(=O)=NC(=O)Cc3c(-c4ccncc4F)cnc4c3CCC4)nn2C1] |
| 451c | 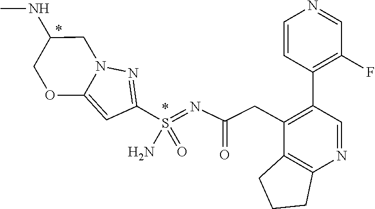 [SMILES: CNC1COc2cc(S(N)(=O)=NC(=O)Cc3c(-c4ccncc4F)cnc4c3CCC4)nn2C1] |
| 451d | 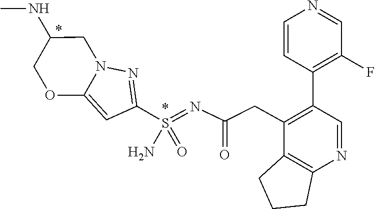 [SMILES: CNC1COc2cc(S(N)(=O)=NC(=O)Cc3c(-c4ccncc4F)cnc4c3CCC4)nn2C1] |
| 452 | 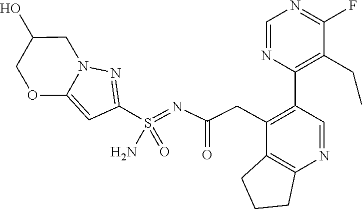 [SMILES: CCc1c(F)ncnc1-c1cnc2c(c1CC(=O)N=S(N)(=O)c1cc3n(n1)CC(O)CO3)CCC2] |
| 452a | 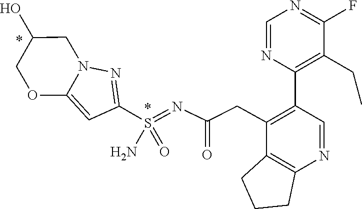 [SMILES: CCc1c(F)ncnc1-c1cnc2c(c1CC(=O)N=S(N)(=O)c1cc3n(n1)CC(O)CO3)CCC2] |
| 452b | 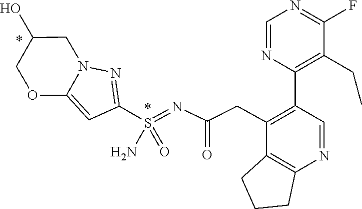 [SMILES: CCc1c(F)ncnc1-c1cnc2c(c1CC(=O)N=S(N)(=O)c1cc3n(n1)CC(O)CO3)CCC2] |
| 452c | 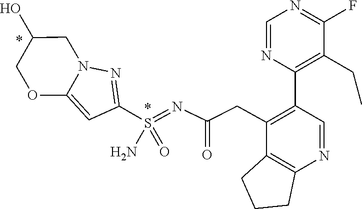 [SMILES: CCc1c(F)ncnc1-c1cnc2c(c1CC(=O)N=S(N)(=O)c1cc3n(n1)CC(O)CO3)CCC2] |
| 452d | 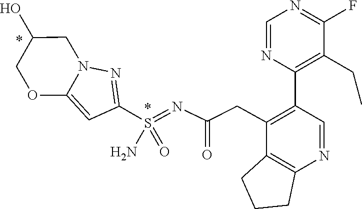 [SMILES: CCc1c(F)ncnc1-c1cnc2c(c1CC(=O)N=S(N)(=O)c1cc3n(n1)CC(O)CO3)CCC2] |
| 453 | 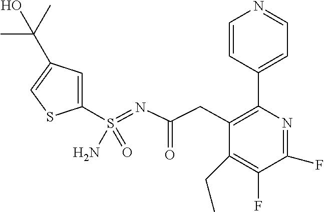 [SMILES: CCc1c(F)c(F)nc(-c2ccncc2)c1CC(=O)N=S(N)(=O)c1cc(C(C)(C)O)cs1] |
| 453a | 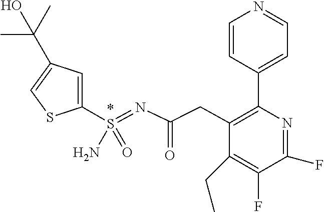 [SMILES: CCc1c(F)c(F)nc(-c2ccncc2)c1CC(=O)N=S(N)(=O)c1cc(C(C)(C)O)cs1] |
| 453b | 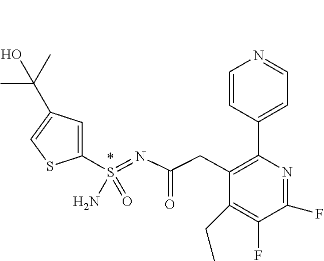 |
| 454 | 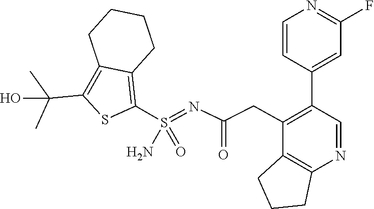 [SMILES: CC(C)(O)c1sc(S(N)(=O)=NC(=O)Cc2c(-c3ccnc(F)c3)cnc3c2CCC3)c2c1CCCC2] |
| 454a | 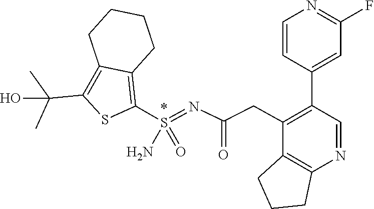 [SMILES: CC(C)(O)c1sc(S(N)(=O)=NC(=O)Cc2c(-c3ccnc(F)c3)cnc3c2CCC3)c2c1CCCC2] |
| 454b | 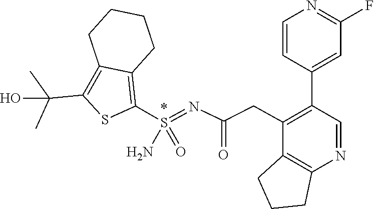 [SMILES: CC(C)(O)c1sc(S(N)(=O)=NC(=O)Cc2c(-c3ccnc(F)c3)cnc3c2CCC3)c2c1CCCC2] |
| 455 | 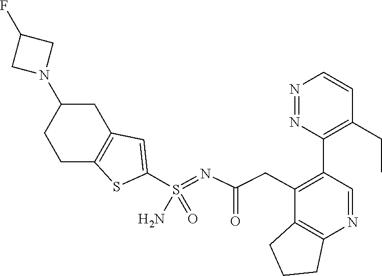 [SMILES: CCc1ccnnc1-c1cnc2c(c1CC(=O)N=S(N)(=O)c1cc3c(s1)CCC(N1CC(F)C1)C3)CCC2] |
| 455a | 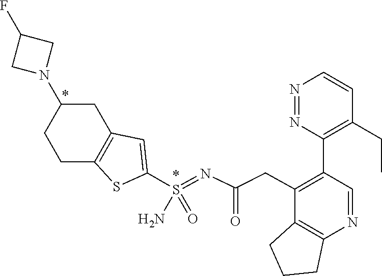 [SMILES: CCc1ccnnc1-c1cnc2c(c1CC(=O)N=S(N)(=O)c1cc3c(s1)CCC(N1CC(F)C1)C3)CCC2] |
| 455b | 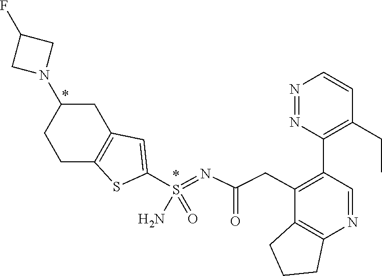 [SMILES: CCc1ccnnc1-c1cnc2c(c1CC(=O)N=S(N)(=O)c1cc3c(s1)CCC(N1CC(F)C1)C3)CCC2] |
| 455c | 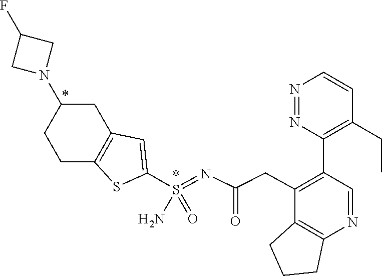 [SMILES: CCc1ccnnc1-c1cnc2c(c1CC(=O)N=S(N)(=O)c1cc3c(s1)CCC(N1CC(F)C1)C3)CCC2] |
| 455d | 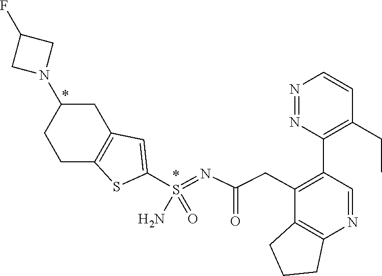 [SMILES: CCc1ccnnc1-c1cnc2c(c1CC(=O)N=S(N)(=O)c1cc3c(s1)CCC(N1CC(F)C1)C3)CCC2] |
| 456 | 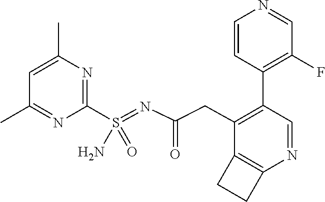 [SMILES: Cc1cc(C)nc(S(N)(=O)=NC(=O)Cc2c(-c3ccncc3F)cnc3c2CC3)n1] |
| 456a | 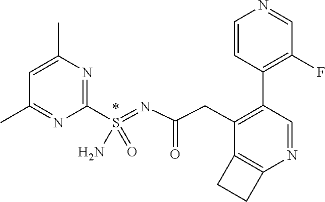 [SMILES: Cc1cc(C)nc(S(N)(=O)=NC(=O)Cc2c(-c3ccncc3F)cnc3c2CC3)n1] |
| 456b | 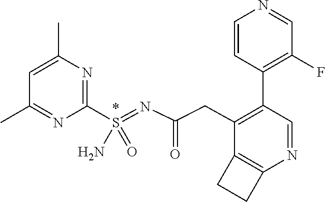 [SMILES: Cc1cc(C)nc(S(N)(=O)=NC(=O)Cc2c(-c3ccncc3F)cnc3c2CC3)n1] |
| 457 | 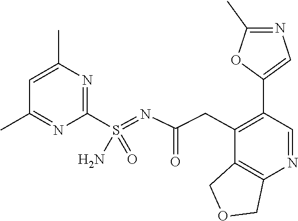 [SMILES: Cc1cc(C)nc(S(N)(=O)=NC(=O)Cc2c(-c3cnc(C)o3)cnc3c2COC3)n1] |
| 457a | 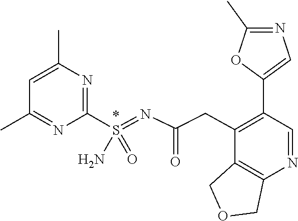 [SMILES: Cc1cc(C)nc(S(N)(=O)=NC(=O)Cc2c(-c3cnc(C)o3)cnc3c2COC3)n1] |
| 457b | 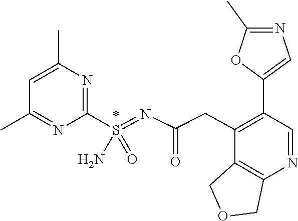 [SMILES: Cc1cc(C)nc(S(N)(=O)=NC(=O)Cc2c(-c3cnc(C)o3)cnc3c2COC3)n1] |
| 458 | 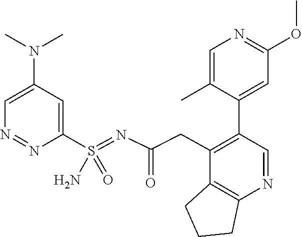 [SMILES: COc1cc(-c2cnc3c(c2CC(=O)N=S(N)(=O)c2cc(N(C)C)cnn2)CCC3)c(C)cn1] |
| 458a | 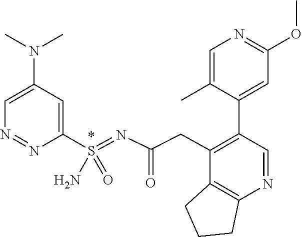 [SMILES: COc1cc(-c2cnc3c(c2CC(=O)N=S(N)(=O)c2cc(N(C)C)cnn2)CCC3)c(C)cn1] |
| 458b | 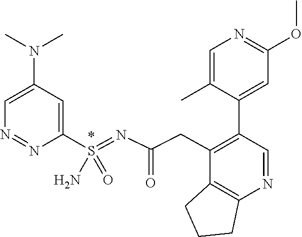 [SMILES: COc1cc(-c2cnc3c(c2CC(=O)N=S(N)(=O)c2cc(N(C)C)cnn2)CCC3)c(C)cn1] |
| 459 | 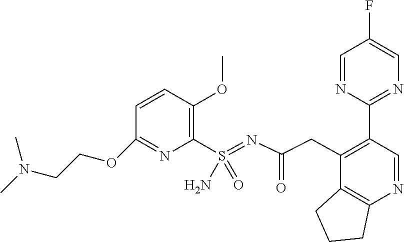 [SMILES: COc1ccc(OCCN(C)C)nc1S(N)(=O)=NC(=O)Cc1c(-c2ncc(F)cn2)cnc2c1CCC2] |
| 459a | 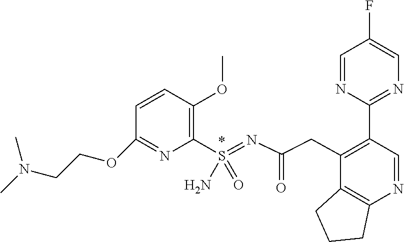 [SMILES: COc1ccc(OCCN(C)C)nc1S(N)(=O)=NC(=O)Cc1c(-c2ncc(F)cn2)cnc2c1CCC2] |
| 459b | 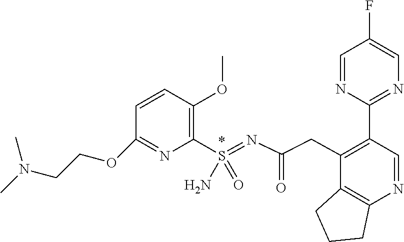 [SMILES: COc1ccc(OCCN(C)C)nc1S(N)(=O)=NC(=O)Cc1c(-c2ncc(F)cn2)cnc2c1CCC2] |
| 460 | 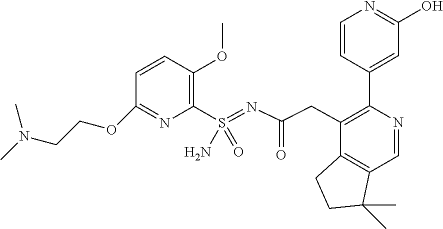 [SMILES: COc1ccc(OCCN(C)C)nc1S(N)(=O)=NC(=O)Cc1c(-c2ccnc(O)c2)ncc2c1CCC2(C)C] |
| 460a | 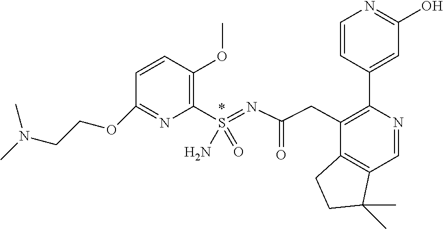 [SMILES: COc1ccc(OCCN(C)C)nc1S(N)(=O)=NC(=O)Cc1c(-c2ccnc(O)c2)ncc2c1CCC2(C)C] |
| 460b | 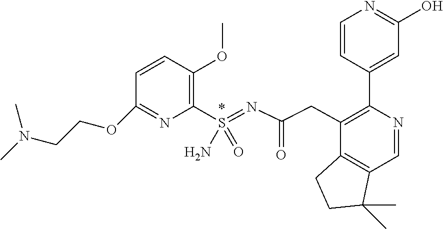 [SMILES: COc1ccc(OCCN(C)C)nc1S(N)(=O)=NC(=O)Cc1c(-c2ccnc(O)c2)ncc2c1CCC2(C)C] |
| 461 | 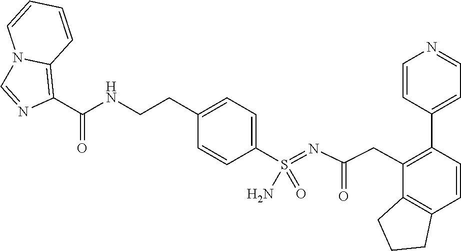 [SMILES: NS(=O)(=NC(=O)Cc1c(-c2ccncc2)ccc2c1CCC2)c1ccc(CCNC(=O)c2ncn3ccccc23)cc1] |
| 461a | 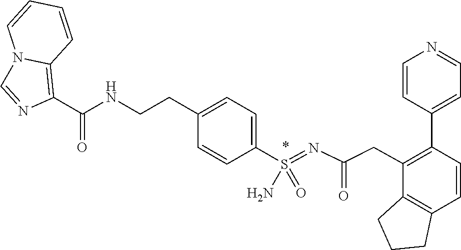 [SMILES: NS(=O)(=NC(=O)Cc1c(-c2ccncc2)ccc2c1CCC2)c1ccc(CCNC(=O)c2ncn3ccccc23)cc1] |
| 461b | 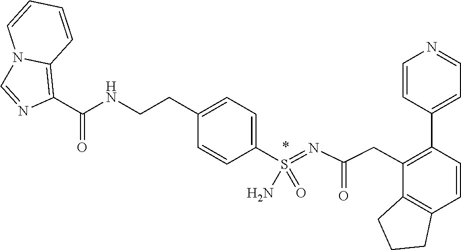 [SMILES: NS(=O)(=NC(=O)Cc1c(-c2ccncc2)ccc2c1CCC2)c1ccc(CCNC(=O)c2ncn3ccccc23)cc1] |
| 462 | 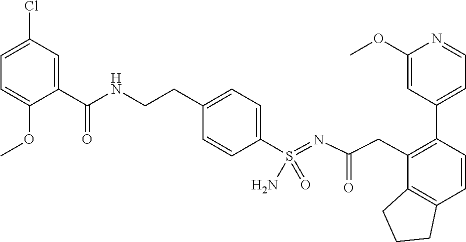 [SMILES: COc1cc(-c2ccc3c(c2CC(=O)N=S(N)(=O)c2ccc(CCNC(=O)c4cc(Cl)ccc4OC)cc2)CCC3)ccn1] |
| 462a | 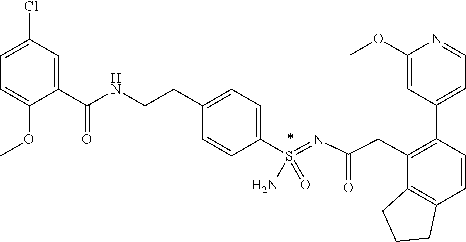 [SMILES: COc1cc(-c2ccc3c(c2CC(=O)N=S(N)(=O)c2ccc(CCNC(=O)c4cc(Cl)ccc4OC)cc2)CCC3)ccn1] |
| 462b | 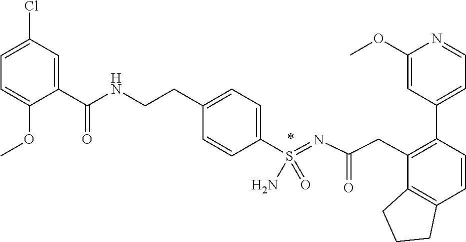 [SMILES: COc1cc(-c2ccc3c(c2CC(=O)N=S(N)(=O)c2ccc(CCNC(=O)c4cc(Cl)ccc4OC)cc2)CCC3)ccn1] |
| 463 | 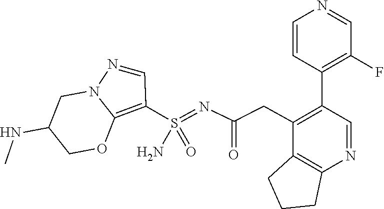 [SMILES: CNC1COc2c(S(N)(=O)=NC(=O)Cc3c(-c4ccncc4F)cnc4c3CCC4)cnn2C1] |
| 463a | 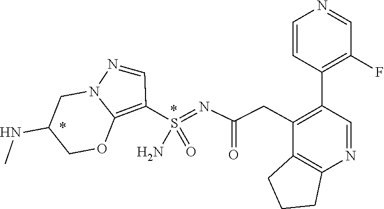 [SMILES: CNC1COc2c(S(N)(=O)=NC(=O)Cc3c(-c4ccncc4F)cnc4c3CCC4)cnn2C1] |
| 463b | 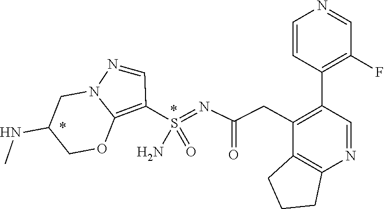 [SMILES: CNC1COc2c(S(N)(=O)=NC(=O)Cc3c(-c4ccncc4F)cnc4c3CCC4)cnn2C1] |
| 463c | 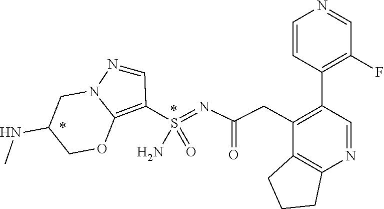 [SMILES: CNC1COc2c(S(N)(=O)=NC(=O)Cc3c(-c4ccncc4F)cnc4c3CCC4)cnn2C1] |
| 463d | 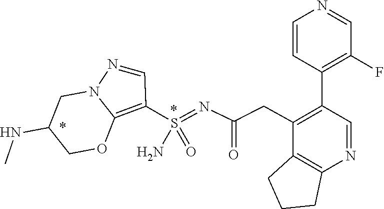 [SMILES: CNC1COc2c(S(N)(=O)=NC(=O)Cc3c(-c4ccncc4F)cnc4c3CCC4)cnn2C1] |
| 464 | 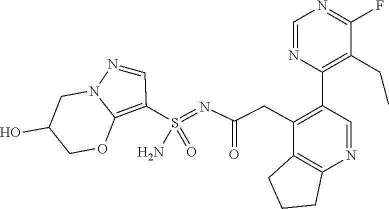 [SMILES: CCc1c(F)ncnc1-c1cnc2c(c1CC(=O)N=S(N)(=O)c1cnn3c1OCC(O)C3)CCC2] |
| 464a | 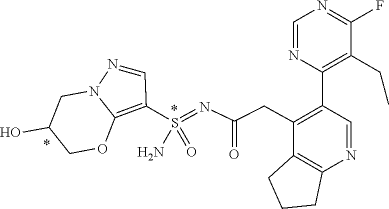 [SMILES: CCc1c(F)ncnc1-c1cnc2c(c1CC(=O)N=S(N)(=O)c1cnn3c1OCC(O)C3)CCC2] |
| 464b | 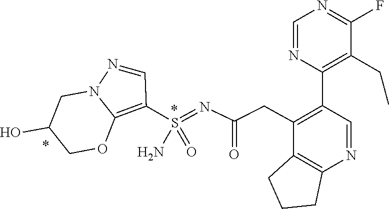 [SMILES: CCc1c(F)ncnc1-c1cnc2c(c1CC(=O)N=S(N)(=O)c1cnn3c1OCC(O)C3)CCC2] |
| 464c | 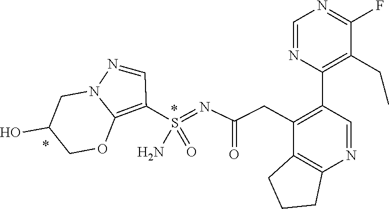 [SMILES: CCc1c(F)ncnc1-c1cnc2c(c1CC(=O)N=S(N)(=O)c1cnn3c1OCC(O)C3)CCC2] |
| 464d | 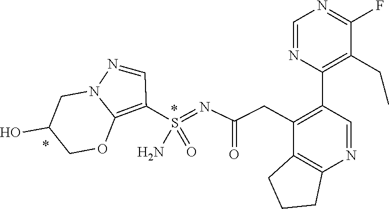 [SMILES: CCc1c(F)ncnc1-c1cnc2c(c1CC(=O)N=S(N)(=O)c1cnn3c1OCC(O)C3)CCC2] |

or a pharmaceutically acceptable salt thereof.

In some embodiments, the compound has Formula AA-B

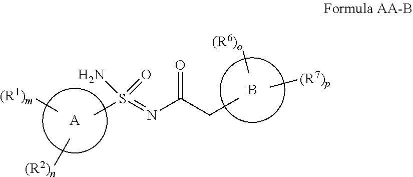

wherein the compound is selected from the group consisting of the compounds in Table 1-5 below:

TABLE 1-5

| | Substituted | Substituted |
|---|---|---|
| Compound # | Ring A | Ring B |
| 501 | (A-1) | (B-1) |
| 502 | (A-1) | (B-2) |
| 503 | (A-1) | (B-3) |
| 504 | (A-1) | (B-4) |
| 505 | (A-1) | (B-5) |
| 506 | (A-1) | (B-6) |
| 507 | (A-1) | (B-7) |
| 508 | (A-1) | (B-8) |
| 509 | (A-1) | (B-9) |
| 510 | (A-1) | (B-10) |
| 511 | (A-1) | (B-11) |
| 512 | (A-1) | (B-12) |
| 513 | (A-1) | (B-13) |
| 514 | (A-1) | (B-14) |
| 515 | (A-1) | (B-15) |
| 516 | (A-2) | (B-1) |
| 517 | (A-2) | (B-2) |
| 518 | (A-2) | (B-3) |
| 519 | (A-2) | (B-4) |
| 520 | (A-2) | (B-5) |
| 521 | (A-2) | (B-6) |
| 522 | (A-2) | (B-7) |
| 523 | (A-2) | (B-8) |
| 524 | (A-2) | (B-9) |
| 525 | (A-2) | (B-10) |
| 526 | (A-2) | (B-11) |
| 527 | (A-2) | (B-12) |
| 528 | (A-2) | (B-13) |
| 529 | (A-2) | (B-14) |
| 530 | (A-2) | (B-15) |
| 531 | (A-3) | (B-1) |
| 532 | (A-3) | (B-2) |
| 533 | (A-3) | (B-3) |
| 534 | (A-3) | (B-4) |
| 535 | (A-3) | (B-5) |
| 536 | (A-3) | (B-6) |
| 537 | (A-3) | (B-7) |
| 538 | (A-3) | (B-8) |
| 539 | (A-3) | (B-9) |
| 540 | (A-3) | (B-10) |
| 541 | (A-3) | (B-11) |
| 542 | (A-3) | (B-12) |
| 543 | (A-3) | (B-13) |
| 544 | (A-3) | (B-14) |
| 545 | (A-3) | (B-15) |
| 546 | (A-4) | (B-1) |
| 547 | (A-4) | (B-2) |
| 548 | (A-4) | (B-3) |
| 549 | (A-4) | (B-4) |
| 550 | (A-4) | (B-5) |
| 551 | (A-4) | (B-6) |
| 552 | (A-4) | (B-7) |
| 553 | (A-4) | (B-8) |
| 554 | (A-4) | (B-9) |
| 555 | (A-4) | (B-10) |
| 556 | (A-4) | (B-11) |
| 557 | (A-4) | (B-12) |
| 558 | (A-4) | (B-13) |
| 559 | (A-4) | (B-14) |
| 560 | (A-4) | (B-15) |
| 561 | (A-5) | (B-1) |
| 562 | (A-5) | (B-2) |
| 563 | (A-5) | (B-3) |
| 564 | (A-5) | (B-4) |
| 565 | (A-5) | (B-5) |
| 566 | (A-5) | (B-6) |
| 567 | (A-5) | (B-7) |
| 568 | (A-5) | (B-8) |
| 569 | (A-5) | (B-9) |
| 570 | (A-5) | (B-10) |
| 571 | (A-5) | (B-11) |
| 572 | (A-5) | (B-12) |
| 573 | (A-5) | (B-13) |
| 574 | (A-5) | (B-14) |
| 575 | (A-5) | (B-15) |
| 576 | (A-6) | (B-1) |
| 577 | (A-6) | (B-2) |
| 578 | (A-6) | (B-3) |
| 579 | (A-6) | (B-4) |
| 580 | (A-6) | (B-5) |
| 581 | (A-6) | (B-6) |
| 582 | (A-6) | (B-7) |
| 583 | (A-6) | (B-8) |
| 584 | (A-6) | (B-9) |
| 585 | (A-6) | (B-10) |
| 586 | (A-6) | (B-11) |
| 587 | (A-6) | (B-12) |
| 588 | (A-6) | (B-13) |
| 589 | (A-6) | (B-14) |
| 590 | (A-6) | (B-15) |
| 591 | (A-7) | (B-1) |
| 592 | (A-7) | (B-2) |
| 593 | (A-7) | (B-3) |
| 594 | (A-7) | (B-4) |
| 595 | (A-7) | (B-5) |
| 596 | (A-7) | (B-6) |
| 597 | (A-7) | (B-7) |
| 598 | (A-7) | (B-8) |
| 599 | (A-7) | (B-9) |
| 600 | (A-7) | (B-10) |
| 601 | (A-7) | (B-11) |
| 602 | (A-7) | (B-12) |
| 603 | (A-7) | (B-13) |
| 604 | (A-7) | (B-14) |
| 605 | (A-7) | (B-15) |
| 606 | (A-8) | (B-1) |
| 607 | (A-8) | (B-2) |
| 608 | (A-8) | (B-3) |
| 609 | (A-8) | (B-4) |
| 610 | (A-8) | (B-5) |
| 611 | (A-8) | (B-6) |
| 612 | (A-8) | (B-7) |
| 613 | (A-8) | (B-8) |
| 614 | (A-8) | (B-9) |
| 615 | (A-8) | (B-10) |
| 616 | (A-8) | (B-11) |
| 617 | (A-8) | (B-12) |
| 618 | (A-8) | (B-13) |
| 619 | (A-8) | (B-14) |
| 620 | (A-8) | (B-15) |
| 621 | (A-9) | (B-1) |
| 622 | (A-9) | (B-2) |
| 623 | (A-9) | (B-3) |
| 624 | (A-9) | (B-4) |
| 625 | (A-9) | (B-5) |
| 626 | (A-9) | (B-6) |
| 627 | (A-9) | (B-7) |
| 628 | (A-9) | (B-8) |
| 629 | (A-9) | (B-9) |
| 630 | (A-9) | (B-10) |
| 631 | (A-9) | (B-11) |
| 632 | (A-9) | (B-12) |
| 633 | (A-9) | (B-13) |
| 634 | (A-9) | (B-14) |
| 635 | (A-9) | (B-15) |
| 636 | (A-10) | (B-1) |
| 637 | (A-10) | (B-2) |
| 638 | (A-10) | (B-3) |
| 639 | (A-10) | (B-4) |
| 640 | (A-10) | (B-5) |
| 641 | (A-10) | (B-6) |
| 642 | (A-10) | (B-7) |
| 643 | (A-10) | (B-8) |
| 644 | (A-10) | (B-9) |
| 645 | (A-10) | (B-10) |
| 646 | (A-10) | (B-11) |
| 647 | (A-10) | (B-12) |
| 648 | (A-10) | (B-13) |
| 649 | (A-10) | (B-14) |
| 650 | (A-10) | (B-15) |
| 651 | (A-11) | (B-1) |
| 652 | (A-11) | (B-2) |
| 653 | (A-11) | (B-3) |
| 654 | (A-11) | (B-4) |
| 655 | (A-11) | (B-5) |
| 656 | (A-11) | (B-6) |
| 657 | (A-11) | (B-7) |
| 658 | (A-11) | (B-8) |
| 659 | (A-11) | (B-9) |
| 660 | (A-11) | (B-10) |
| 661 | (A-11) | (B-11) |
| 662 | (A-11) | (B-12) |
| 663 | (A-11) | (B-13) |
| 664 | (A-11) | (B-14) |
| 665 | (A-11) | (B-15) |
| 666 | (A-12) | (B-1) |
| 667 | (A-12) | (B-2) |
| 668 | (A-12) | (B-3) |
| 669 | (A-12) | (B-4) |
| 670 | (A-12) | (B-5) |
| 671 | (A-12) | (B-6) |
| 672 | (A-12) | (B-7) |
| 673 | (A-12) | (B-8) |
| 674 | (A-12) | (B-9) |
| 675 | (A-12) | (B-10) |
| 676 | (A-12) | (B-11) |
| 677 | (A-12) | (B-12) |
| 678 | (A-12) | (B-13) |
| 679 | (A-12) | (B-14) |
| 680 | (A-12) | (B-15) |
| 681 | (A-13) | (B-1) |
| 682 | (A-13) | (B-2) |
| 683 | (A-13) | (B-3) |
| 684 | (A-13) | (B-4) |
| 685 | (A-13) | (B-5) |
| 686 | (A-13) | (B-6) |
| 687 | (A-13) | (B-7) |
| 688 | (A-13) | (B-8) |
| 689 | (A-13) | (B-9) |
| 690 | (A-13) | (B-10) |
| 691 | (A-13) | (B-11) |
| 692 | (A-13) | (B-12) |
| 693 | (A-13) | (B-13) |
| 694 | (A-13) | (B-14) |
| 695 | (A-13) | (B-15) |
| 696 | (A-14) | (B-1) |
| 697 | (A-14) | (B-2) |
| 698 | (A-14) | (B-3) |
| 699 | (A-14) | (B-4) |
| 700 | (A-14) | (B-5) |
| 701 | (A-14) | (B-6) |
| 702 | (A-14) | (B-7) |
| 703 | (A-14) | (B-8) |
| 704 | (A-14) | (B-9) |
| 705 | (A-14) | (B-10) |
| 706 | (A-14) | (B-11) |
| 707 | (A-14) | (B-12) |
| 708 | (A-14) | (B-13) |
| 709 | (A-14) | (B-14) |
| 710 | (A-14) | (B-15) |
| 711 | (A-15) | (B-1) |
| 712 | (A-15) | (B-2) |
| 713 | (A-15) | (B-3) |
| 714 | (A-15) | (B-4) |
| 715 | (A-15) | (B-5) |
| 716 | (A-15) | (B-6) |
| 717 | (A-15) | (B-7) |
| 718 | (A-15) | (B-8) |
| 719 | (A-15) | (B-9) |
| 720 | (A-15) | (B-10) |
| 721 | (A-15) | (B-11) |
| 722 | (A-15) | (B-12) |
| 723 | (A-15) | (B-13) |
| 724 | (A-15) | (B-14) |
| 725 | (A-15) | (B-15) |
| 726 | (A-16) | (B-1) |
| 727 | (A-16) | (B-2) |
| 728 | (A-16) | (B-3) |
| 729 | (A-16) | (B-4) |
| 730 | (A-16) | (B-5) |
| 731 | (A-16) | (B-6) |
| 732 | (A-16) | (B-7) |
| 733 | (A-16) | (B-8) |
| 734 | (A-16) | (B-9) |
| 735 | (A-16) | (B-10) |
| 736 | (A-16) | (B-11) |
| 737 | (A-16) | (B-12) |
| 738 | (A-16) | (B-13) |
| 739 | (A-16) | (B-14) |
| 740 | (A-16) | (B-15) |
| 741 | (A-17) | (B-1) |
| 742 | (A-17) | (B-2) |
| 743 | (A-17) | (B-3) |
| 744 | (A-17) | (B-4) |
| 745 | (A-17) | (B-5) |
| 746 | (A-17) | (B-6) |
| 747 | (A-17) | (B-7) |
| 748 | (A-17) | (B-8) |
| 749 | (A-17) | (B-9) |
| 750 | (A-17) | (B-10) |
| 751 | (A-17) | (B-11) |
| 752 | (A-17) | (B-12) |
| 753 | (A-17) | (B-13) |
| 754 | (A-17) | (B-14) |
| 755 | (A-17) | (B-15) |
| 756 | (A-18) | (B-1) |
| 757 | (A-18) | (B-2) |
| 758 | (A-18) | (B-3) |
| 759 | (A-18) | (B-4) |
| 760 | (A-18) | (B-5) |
| 761 | (A-18) | (B-6) |
| 762 | (A-18) | (B-7) |
| 763 | (A-18) | (B-8) |
| 764 | (A-18) | (B-9) |
| 765 | (A-18) | (B-10) |
| 766 | (A-18) | (B-11) |
| 767 | (A-18) | (B-12) |
| 768 | (A-18) | (B-13) |
| 769 | (A-18) | (B-14) |
| 770 | (A-18) | (B-15) |
| 771 | (A-19) | (B-1) |
| 772 | (A-19) | (B-2) |
| 773 | (A-19) | (B-3) |
| 774 | (A-19) | (B-4) |
| 775 | (A-19) | (B-5) |
| 776 | (A-19) | (B-6) |
| 777 | (A-19) | (B-7) |
| 778 | (A-19) | (B-8) |
| 779 | (A-19) | (B-9) |
| 780 | (A-19) | (B-10) |
| 781 | (A-19) | (B-11) |
| 782 | (A-19) | (B-12) |
| 783 | (A-19) | (B-13) |
| 784 | (A-19) | (B-14) |
| 785 | (A-19) | (B-15) |
| 786 | (A-20) | (B-1) |
| 787 | (A-20) | (B-2) |
| 788 | (A-20) | (B-3) |
| 789 | (A-20) | (B-4) |
| 790 | (A-20) | (B-5) |
| 791 | (A-20) | (B-6) |
| 792 | (A-20) | (B-7) |
| 793 | (A-20) | (B-8) |
| 794 | (A-20) | (B-9) |
| 795 | (A-20) | (B-10) |
| 796 | (A-20) | (B-11) |
| 797 | (A-20) | (B-12) |
| 798 | (A-20) | (B-13) |
| 799 | (A-20) | (B-14) |
| 800 | (A-20) | (B-15) |
| 801 | (A-21) | (B-1) |
| 802 | (A-21) | (B-2) |
| 803 | (A-21) | (B-3) |
| 804 | (A-21) | (B-4) |
| 805 | (A-21) | (B-5) |
| 806 | (A-21) | (B-6) |
| 807 | (A-21) | (B-7) |
| 808 | (A-21) | (B-8) |
| 809 | (A-21) | (B-9) |
| 810 | (A-21) | (B-10) |
| 811 | (A-21) | (B-11) |
| 812 | (A-21) | (B-12) |
| 813 | (A-21) | (B-13) |
| 814 | (A-21) | (B-14) |
| 815 | (A-21) | (B-15) |
| 816 | (A-22) | (B-1) |
| 817 | (A-22) | (B-2) |
| 818 | (A-22) | (B-3) |
| 819 | (A-22) | (B-4) |
| 820 | (A-22) | (B-5) |
| 821 | (A-22) | (B-6) |
| 822 | (A-22) | (B-7) |
| 823 | (A-22) | (B-8) |
| 824 | (A-22) | (B-9) |
| 825 | (A-22) | (B-10) |
| 826 | (A-22) | (B-11) |
| 827 | (A-22) | (B-12) |
| 828 | (A-22) | (B-13) |
| 829 | (A-22) | (B-14) |
| 830 | (A-22) | (B-15) |
| 831 | (A-23) | (B-1) |
| 832 | (A-23) | (B-2) |
| 833 | (A-23) | (B-3) |
| 834 | (A-23) | (B-4) |
| 835 | (A-23) | (B-5) |
| 836 | (A-23) | (B-6) |
| 837 | (A-23) | (B-7) |
| 838 | (A-23) | (B-8) |
| 839 | (A-23) | (B-9) |
| 840 | (A-23) | (B-10) |
| 841 | (A-23) | (B-11) |
| 842 | (A-23) | (B-12) |
| 843 | (A-23) | (B-13) |
| 844 | (A-23) | (B-14) |
| 845 | (A-23) | (B-15) |
| 846 | (A-24) | (B-1) |
| 847 | (A-24) | (B-2) |
| 848 | (A-24) | (B-3) |
| 849 | (A-24) | (B-4) |
| 850 | (A-24) | (B-5) |
| 851 | (A-24) | (B-6) |
| 852 | (A-24) | (B-7) |
| 853 | (A-24) | (B-8) |
| 854 | (A-24) | (B-9) |
| 855 | (A-24) | (B-10) |
| 856 | (A-24) | (B-11) |
| 857 | (A-24) | (B-12) |
| 858 | (A-24) | (B-13) |
| 859 | (A-24) | (B-14) |
| 860 | (A-24) | (B-15) |
| 861 | (A-25) | (B-1) |
| 862 | (A-25) | (B-2) |
| 863 | (A-25) | (B-3) |
| 864 | (A-25) | (B-4) |
| 865 | (A-25) | (B-5) |
| 866 | (A-25) | (B-6) |
| 867 | (A-25) | (B-7) |
| 868 | (A-25) | (B-8) |
| 869 | (A-25) | (B-9) |
| 870 | (A-25) | (B-10) |
| 871 | (A-25) | (B-11) |
| 872 | (A-25) | (B-12) |
| 873 | (A-25) | (B-13) |
| 874 | (A-25) | (B-14) |
| 875 | (A-25) | (B-15) |
| 876 | (A-26) | (B-1) |
| 877 | (A-26) | (B-2) |
| 878 | (A-26) | (B-3) |
| 879 | (A-26) | (B-4) |
| 880 | (A-26) | (B-5) |
| 881 | (A-26) | (B-6) |
| 882 | (A-26) | (B-7) |
| 883 | (A-26) | (B-8) |
| 884 | (A-26) | (B-9) |
| 885 | (A-26) | (B-10) |
| 886 | (A-26) | (B-11) |
| 887 | (A-26) | (B-12) |
| 888 | (A-26) | (B-13) |
| 889 | (A-26) | (B-14) |
| 890 | (A-26) | (B-15) |
| 891 | (A-27) | (B-1) |
| 892 | (A-27) | (B-2) |
| 893 | (A-27) | (B-3) |
| 894 | (A-27) | (B-4) |
| 895 | (A-27) | (B-5) |
| 896 | (A-27) | (B-6) |
| 897 | (A-27) | (B-7) |
| 898 | (A-27) | (B-8) |
| 899 | (A-27) | (B-9) |
| 900 | (A-27) | (B-10) |
| 901 | (A-27) | (B-11) |
| 902 | (A-27) | (B-12) |
| 903 | (A-27) | (B-13) |
| 904 | (A-27) | (B-14) |
| 905 | (A-27) | (B-15) |
| 906 | (A-28) | (B-1) |
| 907 | (A-28) | (B-2) |
| 908 | (A-28) | (B-3) |
| 909 | (A-28) | (B-4) |
| 910 | (A-28) | (B-5) |
| 911 | (A-28) | (B-6) |
| 912 | (A-28) | (B-7) |
| 913 | (A-28) | (B-8) |
| 914 | (A-28) | (B-9) |
| 915 | (A-28) | (B-10) |
| 916 | (A-28) | (B-11) |
| 917 | (A-28) | (B-12) |
| 918 | (A-28) | (B-13) |
| 919 | (A-28) | (B-14) |
| 920 | (A-28) | (B-15) |
| 921 | (A-29) | (B-1) |
| 922 | (A-29) | (B-2) |
| 923 | (A-29) | (B-3) |
| 924 | (A-29) | (B-4) |
| 925 | (A-29) | (B-5) |
| 926 | (A-29) | (B-6) |
| 927 | (A-29) | (B-7) |
| 928 | (A-29) | (B-8) |
| 929 | (A-29) | (B-9) |
| 930 | (A-29) | (B-10) |
| 931 | (A-29) | (B-11) |
| 932 | (A-29) | (B-12) |
| 933 | (A-29) | (B-13) |
| 934 | (A-29) | (B-14) |
| 935 | (A-29) | (B-15) |
| 936 | (A-30) | (B-1) |
| 937 | (A-30) | (B-2) |
| 938 | (A-30) | (B-3) |
| 939 | (A-30) | (B-4) |
| 940 | (A-30) | (B-5) |
| 941 | (A-30) | (B-6) |
| 942 | (A-30) | (B-7) |
| 943 | (A-30) | (B-8) |
| 944 | (A-30) | (B-9) |
| 945 | (A-30) | (B-10) |
| 946 | (A-30) | (B-11) |
| 947 | (A-30) | (B-12) |
| 948 | (A-30) | (B-13) |
| 949 | (A-30) | (B-14) |
| 950 | (A-30) | (B-15) |
| 951 | (A-31) | (B-1) |
| 952 | (A-31) | (B-2) |
| 953 | (A-31) | (B-3) |
| 954 | (A-31) | (B-4) |
| 955 | (A-31) | (B-5) |
| 956 | (A-31) | (B-6) |
| 957 | (A-31) | (B-7) |
| 958 | (A-31) | (B-8) |
| 959 | (A-31) | (B-9) |
| 960 | (A-31) | (B-10) |
| 961 | (A-31) | (B-11) |
| 962 | (A-31) | (B-12) |
| 963 | (A-31) | (B-13) |
| 964 | (A-31) | (B-14) |
| 965 | (A-31) | (B-15) |
| 966 | (A-32) | (B-1) |
| 967 | (A-32) | (B-2) |
| 968 | (A-32) | (B-3) |
| 969 | (A-32) | (B-4) |
| 970 | (A-32) | (B-5) |
| 971 | (A-32) | (B-6) |
| 972 | (A-32) | (B-7) |
| 973 | (A-32) | (B-8) |
| 974 | (A-32) | (B-9) |
| 975 | (A-32) | (B-10) |
| 976 | (A-32) | (B-11) |
| 977 | (A-32) | (B-12) |
| 978 | (A-32) | (B-13) |
| 979 | (A-32) | (B-14) |
| 980 | (A-32) | (B-15) |
| 981 | (A-33) | (B-1) |
| 982 | (A-33) | (B-2) |
| 983 | (A-33) | (B-3) |
| 984 | (A-33) | (B-4) |
| 985 | (A-33) | (B-5) |
| 986 | (A-33) | (B-6) |
| 987 | (A-33) | (B-7) |
| 988 | (A-33) | (B-8) |
| 989 | (A-33) | (B-9) |
| 990 | (A-33) | (B-10) |
| 991 | (A-33) | (B-11) |
| 992 | (A-33) | (B-12) |
| 993 | (A-33) | (B-13) |
| 994 | (A-33) | (B-14) |
| 995 | (A-33) | (B-15) |
| 996 | (A-34) | (B-1) |
| 997 | (A-34) | (B-2) |
| 998 | (A-34) | (B-3) |
| 999 | (A-34) | (B-4) |
| 1000 | (A-34) | (B-5) |
| 1001 | (A-34) | (B-6) |
| 1002 | (A-34) | (B-7) |
| 1003 | (A-34) | (B-8) |
| 1004 | (A-34) | (B-9) |
| 1005 | (A-34) | (B-10) |
| 1006 | (A-34) | (B-11) |
| 1007 | (A-34) | (B-12) |
| 1008 | (A-34) | (B-13) |
| 1009 | (A-34) | (B-14) |
| 1010 | (A-34) | (B-15) |
| 1011 | (A-35) | (B-1) |
| 1012 | (A-35) | (B-2) |
| 1013 | (A-35) | (B-3) |
| 1014 | (A-35) | (B-4) |
| 1015 | (A-35) | (B-5) |
| 1016 | (A-35) | (B-6) |
| 1017 | (A-35) | (B-7) |
| 1018 | (A-35) | (B-8) |
| 1019 | (A-35) | (B-9) |
| 1020 | (A-35) | (B-10) |
| 1021 | (A-35) | (B-11) |
| 1022 | (A-35) | (B-12) |
| 1023 | (A-35) | (B-13) |
| 1024 | (A-35) | (B-14) |
| 1025 | (A-35) | (B-15) |
| 1026 | (A-36) | (B-1) |
| 1027 | (A-36) | (B-2) |
| 1028 | (A-36) | (B-3) |
| 1029 | (A-36) | (B-4) |
| 1030 | (A-36) | (B-5) |
| 1031 | (A-36) | (B-6) |
| 1032 | (A-36) | (B-7) |
| 1033 | (A-36) | (B-8) |
| 1034 | (A-36) | (B-9) |
| 1035 | (A-36) | (B-10) |
| 1036 | (A-36) | (B-11) |
| 1037 | (A-36) | (B-12) |
| 1038 | (A-36) | (B-13) |
| 1039 | (A-36) | (B-14) |
| 1040 | (A-36) | (B-15) |
| 1041 | (A-37) | (B-1) |
| 1042 | (A-37) | (B-2) |
| 1043 | (A-37) | (B-3) |
| 1044 | (A-37) | (B-4) |
| 1045 | (A-37) | (B-5) |
| 1046 | (A-37) | (B-6) |
| 1047 | (A-37) | (B-7) |
| 1048 | (A-37) | (B-8) |
| 1049 | (A-37) | (B-9) |
| 1050 | (A-37) | (B-10) |
| 1051 | (A-37) | (B-11) |
| 1052 | (A-37) | (B-12) |
| 1053 | (A-37) | (B-13) |
| 1054 | (A-37) | (B-14) |
| 1055 | (A-37) | (B-15) |
| 1056 | (A-38) | (B-1) |
| 1057 | (A-38) | (B-2) |
| 1058 | (A-38) | (B-3) |
| 1059 | (A-38) | (B-4) |
| 1060 | (A-38) | (B-5) |
| 1061 | (A-38) | (B-6) |
| 1062 | (A-38) | (B-7) |
| 1063 | (A-38) | (B-8) |
| 1064 | (A-38) | (B-9) |
| 1065 | (A-38) | (B-10) |
| 1066 | (A-38) | (B-11) |
| 1067 | (A-38) | (B-12) |
| 1068 | (A-38) | (B-13) |
| 1069 | (A-38) | (B-14) |
| 1070 | (A-38) | (B-15) |
| 1071 | (A-39) | (B-1) |
| 1072 | (A-39) | (B-2) |
| 1073 | (A-39) | (B-3) |
| 1074 | (A-39) | (B-4) |
| 1075 | (A-39) | (B-5) |
| 1076 | (A-39) | (B-6) |
| 1077 | (A-39) | (B-7) |
| 1078 | (A-39) | (B-8) |
| 1079 | (A-39) | (B-9) |
| 1080 | (A-39) | (B-10) |
| 1081 | (A-39) | (B-11) |
| 1082 | (A-39) | (B-12) |
| 1083 | (A-39) | (B-13) |
| 1084 | (A-39) | (B-14) |
| 1085 | (A-39) | (B-15) |
| 1086 | (A-40) | (B-1) |
| 1087 | (A-40) | (B-2) |
| 1088 | (A-40) | (B-3) |
| 1089 | (A-40) | (B-4) |
| 1090 | (A-40) | (B-5) |
| 1091 | (A-40) | (B-6) |
| 1092 | (A-40) | (B-7) |
| 1093 | (A-40) | (B-8) |
| 1094 | (A-40) | (B-9) |
| 1095 | (A-40) | (B-10) |
| 1096 | (A-40) | (B-11) |
| 1097 | (A-40) | (B-12) |
| 1098 | (A-40) | (B-13) |
| 1099 | (A-40) | (B-14) |
| 1100 | (A-40) | (B-15) |
| 1101 | (A-41) | (B-1) |
| 1102 | (A-41) | (B-2) |
| 1103 | (A-41) | (B-3) |
| 1104 | (A-41) | (B-4) |
| 1105 | (A-41) | (B-5) |
| 1106 | (A-41) | (B-6) |
| 1107 | (A-41) | (B-7) |
| 1108 | (A-41) | (B-8) |
| 1109 | (A-41) | (B-9) |
| 1110 | (A-41) | (B-10) |
| 1111 | (A-41) | (B-11) |
| 1112 | (A-41) | (B-12) |
| 1113 | (A-41) | (B-13) |
| 1114 | (A-41) | (B-14) |
| 1115 | (A-41) | (B-15) |
| 1116 | (A-42) | (B-1) |
| 1117 | (A-42) | (B-2) |
| 1118 | (A-42) | (B-3) |
| 1119 | (A-42) | (B-4) |
| 1120 | (A-42) | (B-5) |
| 1121 | (A-42) | (B-6) |
| 1122 | (A-42) | (B-7) |
| 1123 | (A-42) | (B-8) |
| 1124 | (A-42) | (B-9) |
| 1125 | (A-42) | (B-10) |
| 1126 | (A-42) | (B-11) |
| 1127 | (A-42) | (B-12) |
| 1128 | (A-42) | (B-13) |
| 1129 | (A-42) | (B-14) |
| 1130 | (A-42) | (B-15) |
| 1131 | (A-43) | (B-1) |
| 1132 | (A-43) | (B-2) |
| 1133 | (A-43) | (B-3) |
| 1134 | (A-43) | (B-4) |
| 1135 | (A-43) | (B-5) |
| 1136 | (A-43) | (B-6) |
| 1137 | (A-43) | (B-7) |
| 1138 | (A-43) | (B-8) |
| 1139 | (A-43) | (B-9) |
| 1140 | (A-43) | (B-10) |
| 1141 | (A-43) | (B-11) |
| 1142 | (A-43) | (B-12) |
| 1143 | (A-43) | (B-13) |
| 1144 | (A-43) | (B-14) |
| 1145 | (A-43) | (B-15) |
| 1146 | (A-44) | (B-1) |
| 1147 | (A-44) | (B-2) |
| 1148 | (A-44) | (B-3) |
| 1149 | (A-44) | (B-4) |
| 1150 | (A-44) | (B-5) |
| 1151 | (A-44) | (B-6) |
| 1152 | (A-44) | (B-7) |
| 1153 | (A-44) | (B-8) |
| 1154 | (A-44) | (B-9) |
| 1155 | (A-44) | (B-10) |
| 1156 | (A-44) | (B-11) |
| 1157 | (A-44) | (B-12) |
| 1158 | (A-44) | (B-13) |
| 1159 | (A-44) | (B-14) |
| 1160 | (A-44) | (B-15) |
| 1161 | (A-45) | (B-1) |
| 1162 | (A-45) | (B-2) |
| 1163 | (A-45) | (B-3) |
| 1164 | (A-45) | (B-4) |
| 1165 | (A-45) | (B-5) |
| 1166 | (A-45) | (B-6) |
| 1167 | (A-45) | (B-7) |
| 1168 | (A-45) | (B-8) |
| 1169 | (A-45) | (B-9) |
| 1170 | (A-45) | (B-10) |
| 1171 | (A-45) | (B-11) |
| 1172 | (A-45) | (B-12) |
| 1173 | (A-45) | (B-13) |
| 1174 | (A-45) | (B-14) |
| 1175 | (A-45) | (B-15) |
| 1176 | (A-46) | (B-1) |
| 1177 | (A-46) | (B-2) |
| 1178 | (A-46) | (B-3) |
| 1179 | (A-46) | (B-4) |
| 1180 | (A-46) | (B-5) |
| 1181 | (A-46) | (B-6) |
| 1182 | (A-46) | (B-7) |
| 1183 | (A-46) | (B-8) |
| 1184 | (A-46) | (B-9) |
| 1185 | (A-46) | (B-10) |
| 1186 | (A-46) | (B-11) |
| 1187 | (A-46) | (B-12) |
| 1188 | (A-46) | (B-13) |
| 1189 | (A-46) | (B-14) |
| 1190 | (A-46) | (B-15) |
| 1191 | (A-47) | (B-1) |
| 1192 | (A-47) | (B-2) |
| 1193 | (A-47) | (B-3) |
| 1194 | (A-47) | (B-4) |
| 1195 | (A-47) | (B-5) |
| 1196 | (A-47) | (B-6) |
| 1197 | (A-47) | (B-7) |
| 1198 | (A-47) | (B-8) |
| 1199 | (A-47) | (B-9) |
| 1200 | (A-47) | (B-10) |
| 1201 | (A-47) | (B-11) |
| 1202 | (A-47) | (B-12) |
| 1203 | (A-47) | (B-13) |
| 1204 | (A-47) | (B-14) |
| 1205 | (A-47) | (B-15) |
| 1206 | (A-48) | (B-1) |
| 1207 | (A-48) | (B-2) |
| 1208 | (A-48) | (B-3) |
| 1209 | (A-48) | (B-4) |
| 1210 | (A-48) | (B-5) |
| 1211 | (A-48) | (B-6) |
| 1212 | (A-48) | (B-7) |
| 1213 | (A-48) | (B-8) |
| 1214 | (A-48) | (B-9) |
| 1215 | (A-48) | (B-10) |
| 1216 | (A-48) | (B-11) |
| 1217 | (A-48) | (B-12) |
| 1218 | (A-48) | (B-13) |
| 1219 | (A-48) | (B-14) |
| 1220 | (A-48) | (B-15) |
| 1221 | (A-49) | (B-1) |
| 1222 | (A-49) | (B-2) |
| 1223 | (A-49) | (B-3) |
| 1224 | (A-49) | (B-4) |
| 1225 | (A-49) | (B-5) |
| 1226 | (A-49) | (B-6) |
| 1227 | (A-49) | (B-7) |
| 1228 | (A-49) | (B-8) |
| 1229 | (A-49) | (B-9) |
| 1230 | (A-49) | (B-10) |
| 1231 | (A-49) | (B-11) |
| 1232 | (A-49) | (B-12) |
| 1233 | (A-49) | (B-13) |
| 1234 | (A-49) | (B-14) |
| 1235 | (A-49) | (B-15) |
| 1236 | (A-50) | (B-1) |
| 1237 | (A-50) | (B-2) |
| 1238 | (A-50) | (B-3) |
| 1239 | (A-50) | (B-4) |
| 1240 | (A-50) | (B-5) |
| 1241 | (A-50) | (B-6) |
| 1242 | (A-50) | (B-7) |
| 1243 | (A-50) | (B-8) |
| 1244 | (A-50) | (B-9) |
| 1245 | (A-50) | (B-10) |
| 1246 | (A-50) | (B-11) |
| 1247 | (A-50) | (B-12) |
| 1248 | (A-50) | (B-13) |
| 1249 | (A-50) | (B-14) |
| 1250 | (A-50) | (B-15) |
| 1251 | (A-51) | (B-1) |
| 1252 | (A-51) | (B-2) |
| 1253 | (A-51) | (B-3) |
| 1254 | (A-51) | (B-4) |
| 1255 | (A-51) | (B-5) |
| 1256 | (A-51) | (B-6) |
| 1257 | (A-51) | (B-7) |
| 1258 | (A-51) | (B-8) |
| 1259 | (A-51) | (B-9) |
| 1260 | (A-51) | (B-10) |
| 1261 | (A-51) | (B-11) |
| 1262 | (A-51) | (B-12) |
| 1263 | (A-51) | (B-13) |
| 1264 | (A-51) | (B-14) |
| 1265 | (A-51) | (B-15). |

In some embodiments, provided herein is a compound that is selected from the group consisting of the compounds in Table 1-6:

TABLE 1-6

| Cmpd # | Structure |
|---|---|
| 205a | 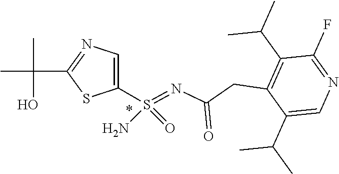 [SMILES: CC(C)c1cnc(F)c(C(C)C)c1CC(=O)N=S(N)(=O)c1cnc(C(C)(C)O)s1] |
| 205b | 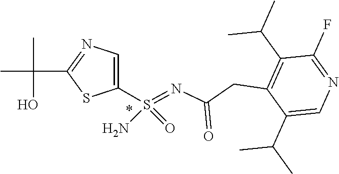 [SMILES: CC(C)c1cnc(F)c(C(C)C)c1CC(=O)N=S(N)(=O)c1cnc(C(C)(C)O)s1] |
| 222a | 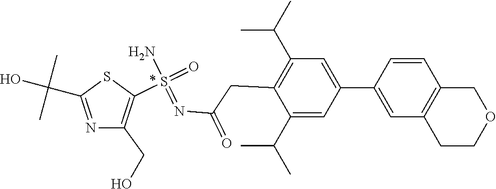 [SMILES: CC(C)c1cc(-c2ccc3c(c2)CCOC3)cc(C(C)C)c1CC(=O)N=S(N)(=O)c1sc(C(C)(C)O)nc1CO] |
| 222b | 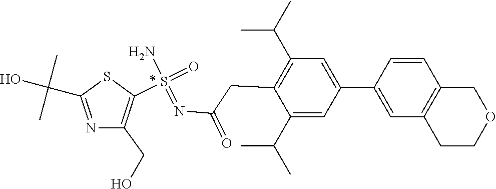 [SMILES: CC(C)c1cc(-c2ccc3c(c2)CCOC3)cc(C(C)C)c1CC(=O)N=S(N)(=O)c1sc(C(C)(C)O)nc1CO] |
| 265b | 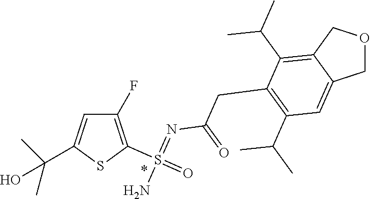 [SMILES: CC(C)c1cc2c(c(C(C)C)c1CC(=O)N=S(N)(=O)c1sc(C(C)(C)O)cc1F)COC2] |
| 305a | 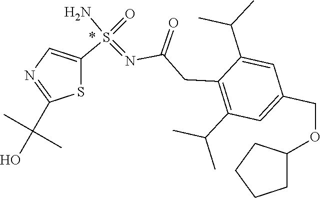 [SMILES: CC(C)c1cc(COC2CCCC2)cc(C(C)C)c1CC(=O)N=S(N)(=O)c1cnc(C(C)(C)O)s1] |
| 305b | 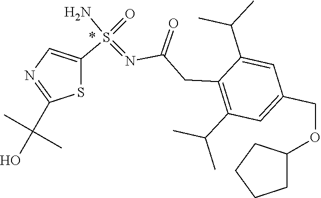 [SMILES: CC(C)c1cc(COC2CCCC2)cc(C(C)C)c1CC(=O)N=S(N)(=O)c1cnc(C(C)(C)O)s1] |
| 501a | 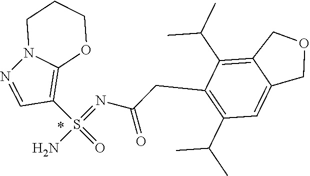 [SMILES: CC(C)c1cc2c(c(C(C)C)c1CC(=O)N=S(N)(=O)c1cnn3c1OCCC3)COC2] |
| 501b | 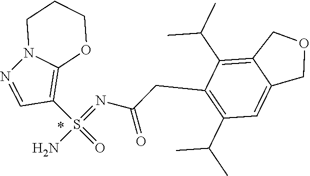 [SMILES: CC(C)c1cc2c(c(C(C)C)c1CC(=O)N=S(N)(=O)c1cnn3c1OCCC3)COC2] |
| 504a | 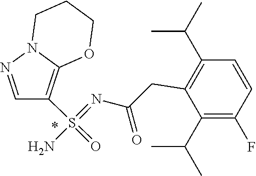 [SMILES: CC(C)c1ccc(F)c(C(C)C)c1CC(=O)N=S(N)(=O)c1cnn2c1OCCC2] |
| 504b | 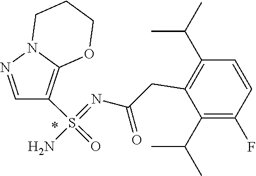 [SMILES: CC(C)c1ccc(F)c(C(C)C)c1CC(=O)N=S(N)(=O)c1cnn2c1OCCC2] |
| 509a | 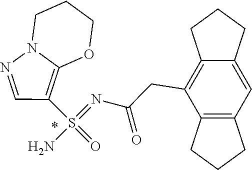 [SMILES: NS(=O)(=NC(=O)Cc1c2c(cc3c1CCC3)CCC2)c1cnn2c1OCCC2] |
| 509b | 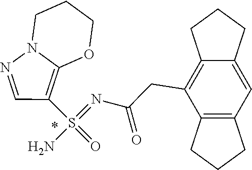 [SMILES: NS(=O)(=NC(=O)Cc1c2c(cc3c1CCC3)CCC2)c1cnn2c1OCCC2] |
| 1301 | 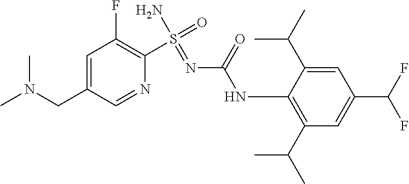 [SMILES: CC(C)c1cc(C(F)F)cc(C(C)C)c1NC(=O)N=S(N)(=O)c1ncc(CN(C)C)cc1F] |
| 1302 | 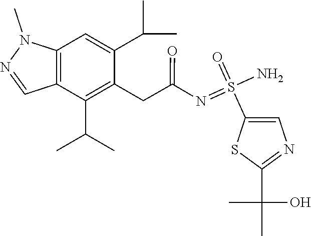 [SMILES: CC(C)c1cc2c(cnn2C)c(C(C)C)c1CC(=O)N=S(N)(=O)c1cnc(C(C)(C)O)s1] |
| 1302b | 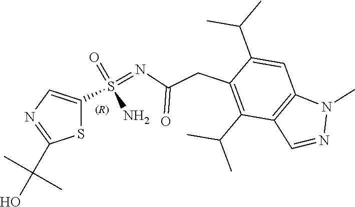 |
| 1303 | 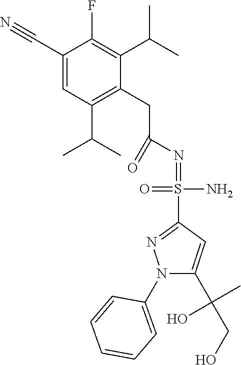 [SMILES: CC(C)c1cc(C#N)c(F)c(C(C)C)c1CC(=O)N=S(N)(=O)c1cc(C(C)(O)CO)n(-c2ccccc2)n1] |
| 1304 | 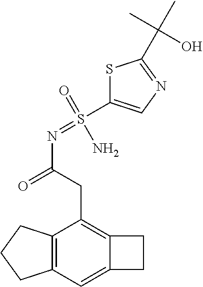 [SMILES: CC(C)(O)c1ncc(S(N)(=O)=NC(=O)Cc2c3c(cc4c2CC4)CCC3)s1] |
| 1304a | 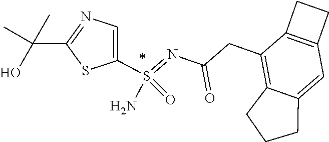 [SMILES: CC(C)(O)c1ncc(S(N)(=O)=NC(=O)Cc2c3c(cc4c2CC4)CCC3)s1] |
| 1304b | 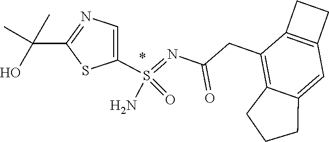 [SMILES: CC(C)(O)c1ncc(S(N)(=O)=NC(=O)Cc2c3c(cc4c2CC4)CCC3)s1] |
| 1305 | 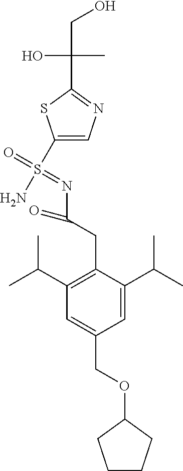 [SMILES: CC(C)c1cc(COC2CCCC2)cc(C(C)C)c1CC(=O)N=S(N)(=O)c1cnc(C(C)(O)CO)s1] |
| 1306 | 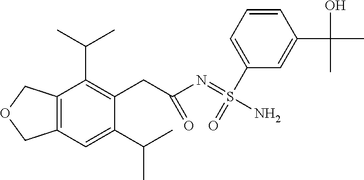 [SMILES: CC(C)c1cc2c(c(C(C)C)c1CC(=O)N=S(N)(=O)c1cccc(C(C)(C)O)c1)COC2] |
| 1306a | 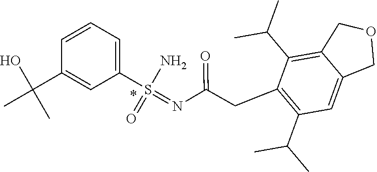 [SMILES: CC(C)c1cc2c(c(C(C)C)c1CC(=O)N=S(N)(=O)c1cccc(C(C)(C)O)c1)COC2] |
| 1306b | 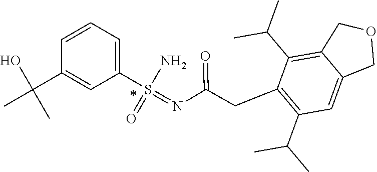 [SMILES: CC(C)c1cc2c(c(C(C)C)c1CC(=O)N=S(N)(=O)c1cccc(C(C)(C)O)c1)COC2] |
| 1307 | 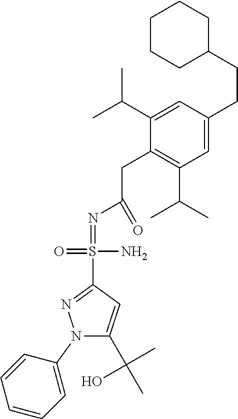 [SMILES: CC(C)c1cc(CCC2CCCCC2)cc(C(C)C)c1CC(=O)N=S(N)(=O)c1cc(C(C)(C)O)n(-c2ccccc2)n1] |
| 1307a | 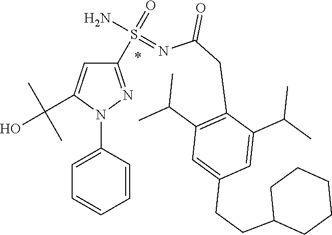 [SMILES: CC(C)c1cc(CCC2CCCCC2)cc(C(C)C)c1CC(=O)N=S(N)(=O)c1cc(C(C)(C)O)n(-c2ccccc2)n1] |
| 1307b | 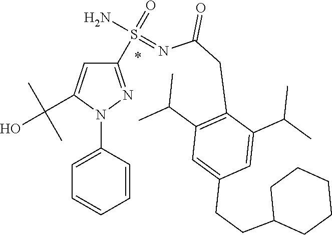 [SMILES: CC(C)c1cc(CCC2CCCCC2)cc(C(C)C)c1CC(=O)N=S(N)(=O)c1cc(C(C)(C)O)n(-c2ccccc2)n1] |
| 1308 | 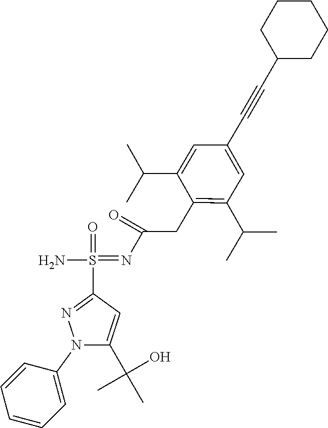 [SMILES: CC(C)c1cc(C#CC2CCCCC2)cc(C(C)C)c1CC(=O)N=S(N)(=O)c1cc(C(C)(C)O)n(-c2ccccc2)n1] |
| 1308a | 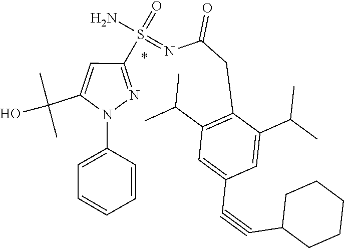 [SMILES: CC(C)c1cc(C#CC2CCCCC2)cc(C(C)C)c1CC(=O)N=S(N)(=O)c1cc(C(C)(C)O)n(-c2ccccc2)n1] |
| 1308b | 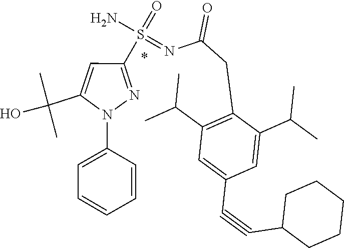 [SMILES: CC(C)c1cc(C#CC2CCCCC2)cc(C(C)C)c1CC(=O)N=S(N)(=O)c1cc(C(C)(C)O)n(-c2ccccc2)n1] |
| 1309 | 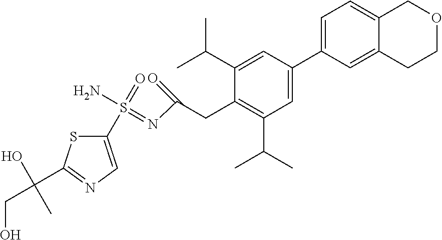 |
| 1310 | 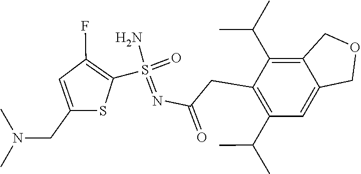 [SMILES: CC(C)c1cc2c(c(C(C)C)c1CC(=O)N=S(N)(=O)c1sc(CN(C)C)cc1F)COC2] |
| 1310a | 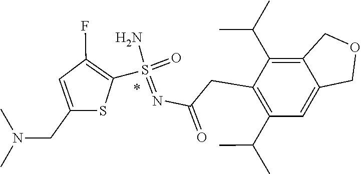 [SMILES: CC(C)c1cc2c(c(C(C)C)c1CC(=O)N=S(N)(=O)c1sc(CN(C)C)cc1F)COC2] |
| 1310b | 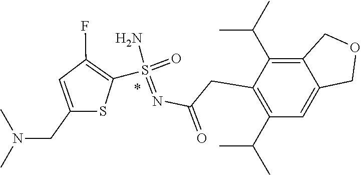 [SMILES: CC(C)c1cc2c(c(C(C)C)c1CC(=O)N=S(N)(=O)c1sc(CN(C)C)cc1F)COC2] |
| 1311 | 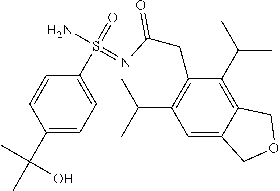 [SMILES: CC(C)c1cc2c(c(C(C)C)c1CC(=O)N=S(N)(=O)c1ccc(C(C)(C)O)cc1)COC2] |
| 1311a | 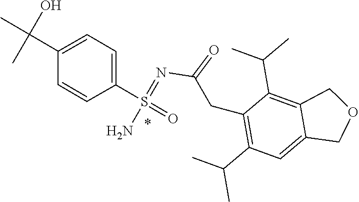 [SMILES: CC(C)c1cc2c(c(C(C)C)c1CC(=O)N=S(N)(=O)c1ccc(C(C)(C)O)cc1)COC2] |
| 1311b | 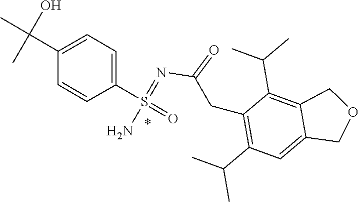 [SMILES: CC(C)c1cc2c(c(C(C)C)c1CC(=O)N=S(N)(=O)c1ccc(C(C)(C)O)cc1)COC2] |
| 1312 | 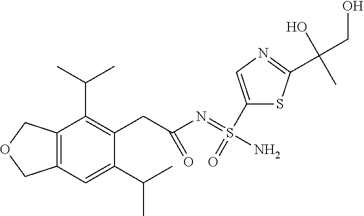 [SMILES: CC(C)c1cc2c(c(C(C)C)c1CC(=O)N=S(N)(=O)c1cnc(C(C)(O)CO)s1)COC2] |
| 1312a | 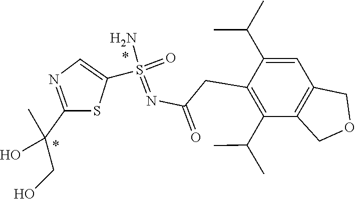 [SMILES: CC(C)c1cc2c(c(C(C)C)c1CC(=O)N=S(N)(=O)c1cnc(C(C)(O)CO)s1)COC2] |
| 1312b | 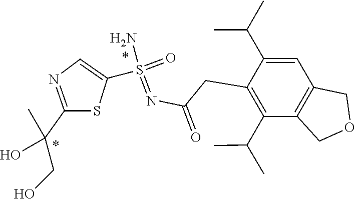 [SMILES: CC(C)c1cc2c(c(C(C)C)c1CC(=O)N=S(N)(=O)c1cnc(C(C)(O)CO)s1)COC2] |
| 1313 | 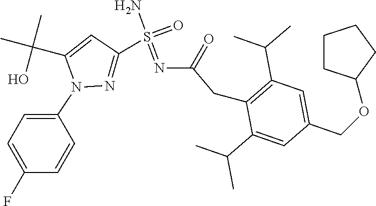 [SMILES: CC(C)c1cc(COC2CCCC2)cc(C(C)C)c1CC(=O)N=S(N)(=O)c1cc(C(C)(C)O)n(-c2ccc(F)cc2)n1] |
| 1313a | 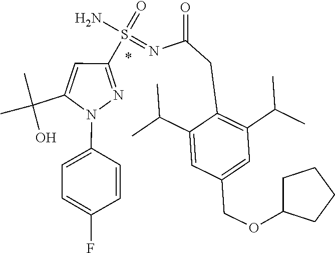 [SMILES: CC(C)c1cc(COC2CCCC2)cc(C(C)C)c1CC(=O)N=S(N)(=O)c1cc(C(C)(C)O)n(-c2ccc(F)cc2)n1] |
| 1313b | 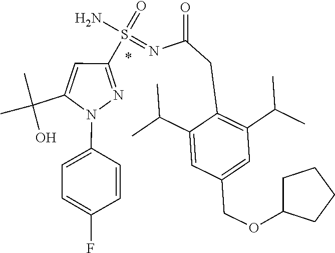 [SMILES: CC(C)c1cc(COC2CCCC2)cc(C(C)C)c1CC(=O)N=S(N)(=O)c1cc(C(C)(C)O)n(-c2ccc(F)cc2)n1] |
| 1314 | 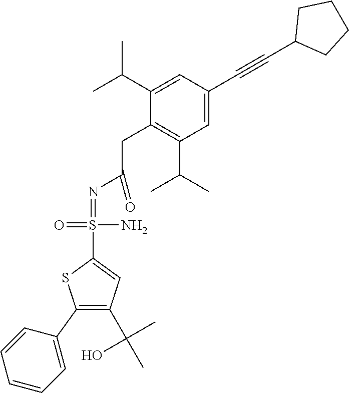 [SMILES: CC(C)c1cc(C#CC2CCCC2)cc(C(C)C)c1CC(=O)N=S(N)(=O)c1cc(C(C)(C)O)c(-c2ccccc2)s1] |
| 1315 | 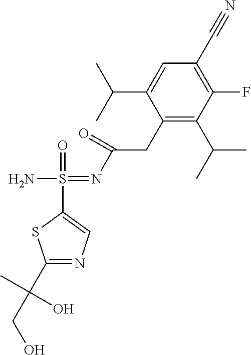 [SMILES: CC(C)c1cc(C#N)c(F)c(C(C)C)c1CC(=O)N=S(N)(=O)c1cnc(C(C)(O)CO)s1] |
| 1315a | 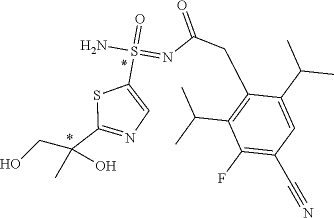 [SMILES: CC(C)c1cc(C#N)c(F)c(C(C)C)c1CC(=O)N=S(N)(=O)c1cnc(C(C)(O)CO)s1] |
| 1315b | 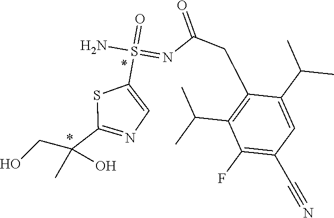 [SMILES: CC(C)c1cc(C#N)c(F)c(C(C)C)c1CC(=O)N=S(N)(=O)c1cnc(C(C)(O)CO)s1] |
| 1315d | 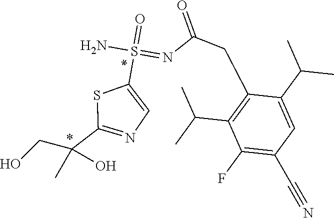 [SMILES: CC(C)c1cc(C#N)c(F)c(C(C)C)c1CC(=O)N=S(N)(=O)c1cnc(C(C)(O)CO)s1] |
| 1315e | 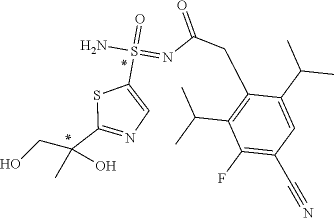 [SMILES: CC(C)c1cc(C#N)c(F)c(C(C)C)c1CC(=O)N=S(N)(=O)c1cnc(C(C)(O)CO)s1] |
| 1315f | 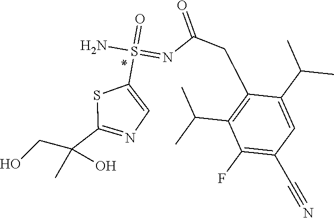 [SMILES: CC(C)c1cc(C#N)c(F)c(C(C)C)c1CC(=O)N=S(N)(=O)c1cnc(C(C)(O)CO)s1] |
| 1316 | 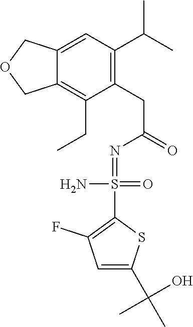 [SMILES: CCc1c2c(cc(C(C)C)c1CC(=O)N=S(N)(=O)c1sc(C(C)(C)O)cc1F)COC2] |
| 1316a | 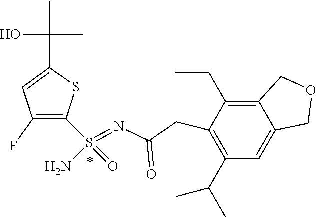 [SMILES: CCc1c2c(cc(C(C)C)c1CC(=O)N=S(N)(=O)c1sc(C(C)(C)O)cc1F)COC2] |
| 1316b | 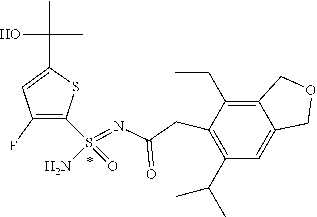 [SMILES: CCc1c2c(cc(C(C)C)c1CC(=O)N=S(N)(=O)c1sc(C(C)(C)O)cc1F)COC2] |
| 1317 | 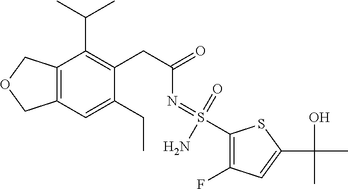 [SMILES: CCc1cc2c(c(C(C)C)c1CC(=O)N=S(N)(=O)c1sc(C(C)(C)O)cc1F)COC2] |
| 1317a | 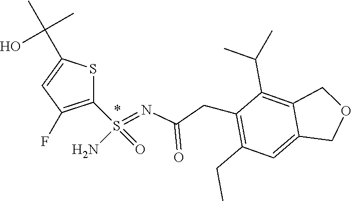 [SMILES: CCc1cc2c(c(C(C)C)c1CC(=O)N=S(N)(=O)c1sc(C(C)(C)O)cc1F)COC2] |
| 1317b | 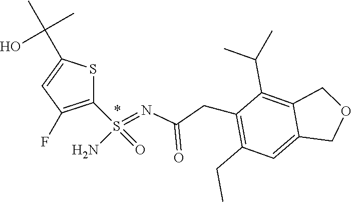 [SMILES: CCc1cc2c(c(C(C)C)c1CC(=O)N=S(N)(=O)c1sc(C(C)(C)O)cc1F)COC2] |
| 1318 | 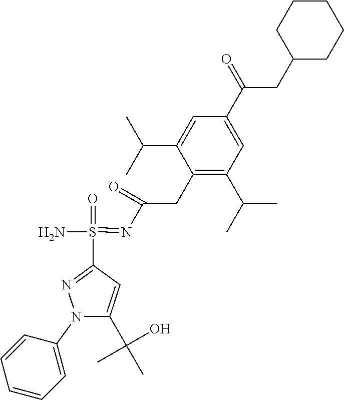 [SMILES: CC(C)c1cc(C(=O)CC2CCCCC2)cc(C(C)C)c1CC(=O)N=S(N)(=O)c1cc(C(C)(C)O)n(-c2ccccc2)n1] |
| 1318a | 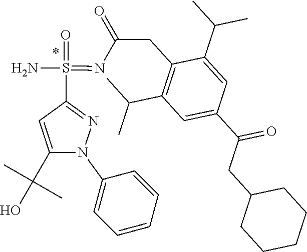 [SMILES: CC(C)c1cc(C(=O)CC2CCCCC2)cc(C(C)C)c1CC(=O)N=S(N)(=O)c1cc(C(C)(C)O)n(-c2ccccc2)n1] |
| 1318b | 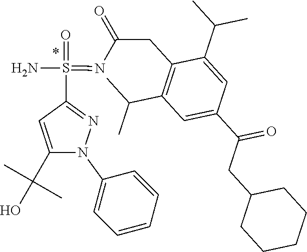 [SMILES: CC(C)c1cc(C(=O)CC2CCCCC2)cc(C(C)C)c1CC(=O)N=S(N)(=O)c1cc(C(C)(C)O)n(-c2ccccc2)n1] |
| 1319 | 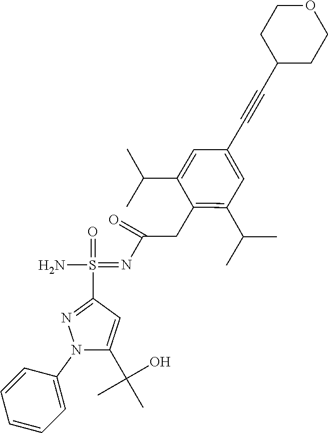 [SMILES: CC(C)c1cc(C#CC2CCOCC2)cc(C(C)C)c1CC(=O)N=S(N)(=O)c1cc(C(C)(C)O)n(-c2ccccc2)n1] |
| 1319a | 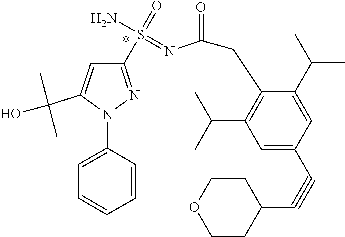 [SMILES: CC(C)c1cc(C#CC2CCOCC2)cc(C(C)C)c1CC(=O)N=S(N)(=O)c1cc(C(C)(C)O)n(-c2ccccc2)n1] |
| 1319b | 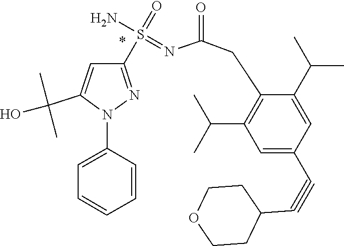 [SMILES: CC(C)c1cc(C#CC2CCOCC2)cc(C(C)C)c1CC(=O)N=S(N)(=O)c1cc(C(C)(C)O)n(-c2ccccc2)n1] |
| 1320 | 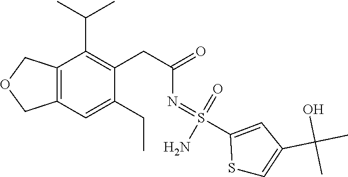 [SMILES: CCc1cc2c(c(C(C)C)c1CC(=O)N=S(N)(=O)c1cc(C(C)(C)O)cs1)COC2] |
| 1320a | 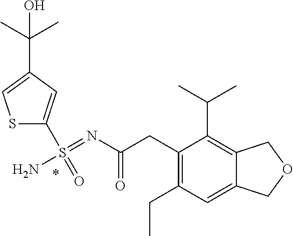 [SMILES: CCc1cc2c(c(C(C)C)c1CC(=O)N=S(N)(=O)c1cc(C(C)(C)O)cs1)COC2] |
| 1320b | 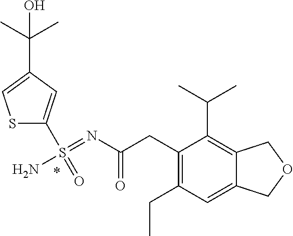 [SMILES: CCc1cc2c(c(C(C)C)c1CC(=O)N=S(N)(=O)c1cc(C(C)(C)O)cs1)COC2] |
| 1321 | 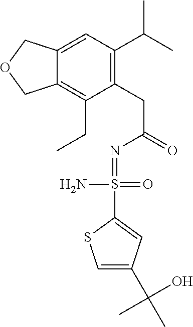 [SMILES: CCc1c2c(cc(C(C)C)c1CC(=O)N=S(N)(=O)c1cc(C(C)(C)O)cs1)COC2] |
| 1321a | 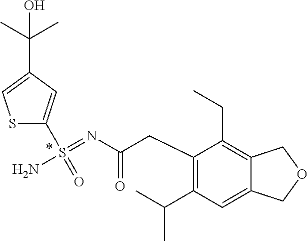 [SMILES: CCc1c2c(cc(C(C)C)c1CC(=O)N=S(N)(=O)c1cc(C(C)(C)O)cs1)COC2] |
| 1321b | 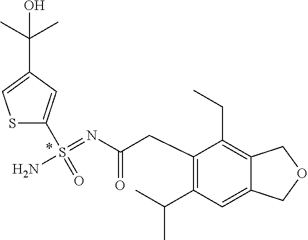 [SMILES: CCc1c2c(cc(C(C)C)c1CC(=O)N=S(N)(=O)c1cc(C(C)(C)O)cs1)COC2] |
| 1322 | 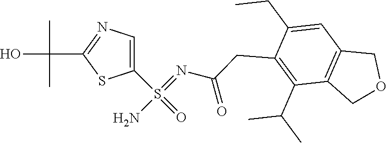 [SMILES: CCc1cc2c(c(C(C)C)c1CC(=O)N=S(N)(=O)c1cnc(C(C)(C)O)s1)COC2] |
| 1322a | 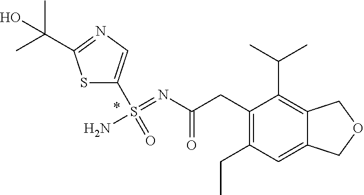 [SMILES: CCc1cc2c(c(C(C)C)c1CC(=O)N=S(N)(=O)c1cnc(C(C)(C)O)s1)COC2] |
| 1322b | 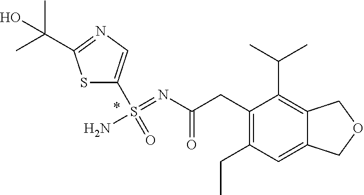 [SMILES: CCc1cc2c(c(C(C)C)c1CC(=O)N=S(N)(=O)c1cnc(C(C)(C)O)s1)COC2] |
| 1323 | 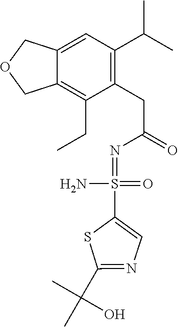 [SMILES: CCc1c2c(cc(C(C)C)c1CC(=O)N=S(N)(=O)c1cnc(C(C)(C)O)s1)COC2] |
| 1323a | 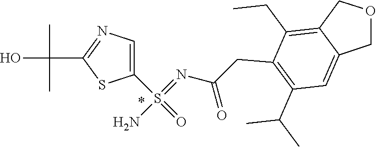 [SMILES: CCc1c2c(cc(C(C)C)c1CC(=O)N=S(N)(=O)c1cnc(C(C)(C)O)s1)COC2] |
| 1323b | 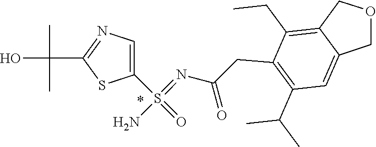 [SMILES: CCc1c2c(cc(C(C)C)c1CC(=O)N=S(N)(=O)c1cnc(C(C)(C)O)s1)COC2] |
| 1324 | 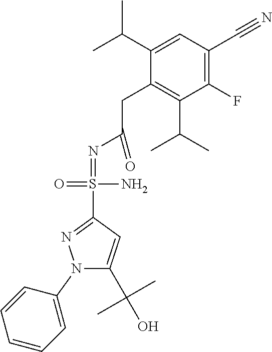 [SMILES: CC(C)c1cc(C#N)c(F)c(C(C)C)c1CC(=O)N=S(N)(=O)c1cc(C(C)(C)O)n(-c2ccccc2)n1] |
| 1324a | 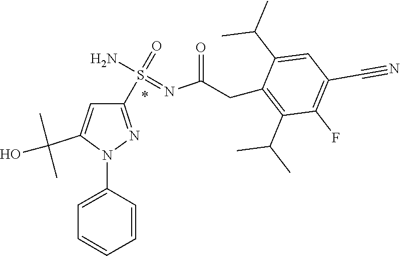 [SMILES: CC(C)c1cc(C#N)c(F)c(C(C)C)c1CC(=O)N=S(N)(=O)c1cc(C(C)(C)O)n(-c2ccccc2)n1] |
| 1324b | 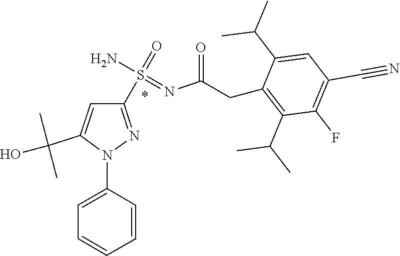 [SMILES: CC(C)c1cc(C#N)c(F)c(C(C)C)c1CC(=O)N=S(N)(=O)c1cc(C(C)(C)O)n(-c2ccccc2)n1] |
| 1325 | 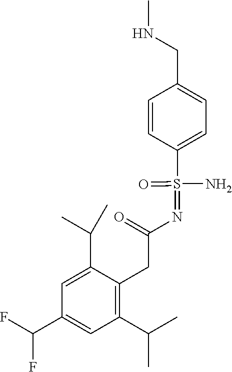 [SMILES: CNCc1ccc(S(N)(=O)=NC(=O)Cc2c(C(C)C)cc(C(F)F)cc2C(C)C)cc1] |
| 1325a | 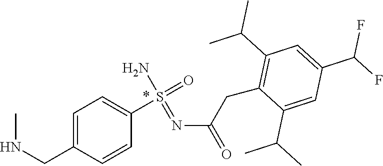 [SMILES: CNCc1ccc(S(N)(=O)=NC(=O)Cc2c(C(C)C)cc(C(F)F)cc2C(C)C)cc1] |
| 1325b | 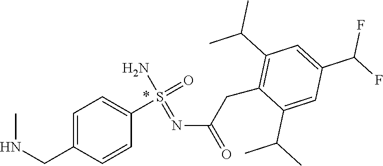 [SMILES: CNCc1ccc(S(N)(=O)=NC(=O)Cc2c(C(C)C)cc(C(F)F)cc2C(C)C)cc1] |
| 1326 | 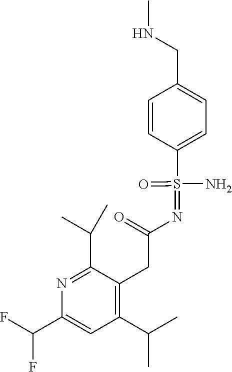 [SMILES: CNCc1ccc(S(N)(=O)=NC(=O)Cc2c(C(C)C)cc(C(F)F)nc2C(C)C)cc1] |
| 1326a | 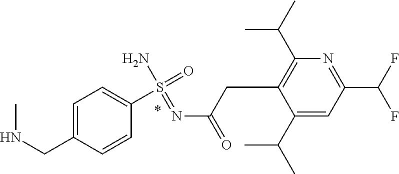 [SMILES: CNCc1ccc(S(N)(=O)=NC(=O)Cc2c(C(C)C)cc(C(F)F)nc2C(C)C)cc1] |
| 1326b | 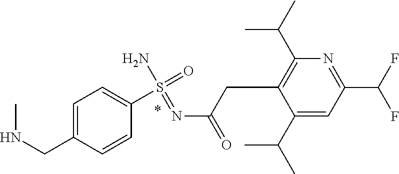 [SMILES: CNCc1ccc(S(N)(=O)=NC(=O)Cc2c(C(C)C)cc(C(F)F)nc2C(C)C)cc1] |
| 1327 | 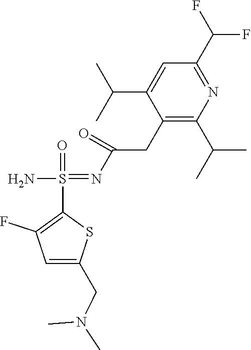 [SMILES: CC(C)c1cc(C(F)F)nc(C(C)C)c1CC(=O)N=S(N)(=O)c1sc(CN(C)C)cc1F] |
| 1327a | 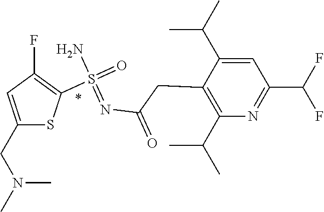 [SMILES: CC(C)c1cc(C(F)F)nc(C(C)C)c1CC(=O)N=S(N)(=O)c1sc(CN(C)C)cc1F] |
| 1327b | 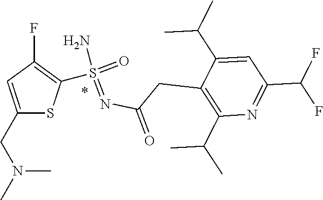 [SMILES: CC(C)c1cc(C(F)F)nc(C(C)C)c1CC(=O)N=S(N)(=O)c1sc(CN(C)C)cc1F] |
| 1328 | 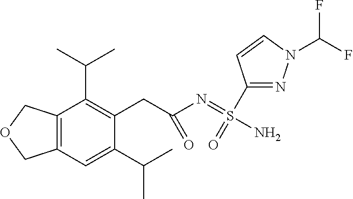 [SMILES: CC(C)c1cc2c(c(C(C)C)c1CC(=O)N=S(N)(=O)c1ccn(C(F)F)n1)COC2] |
| 1328a | 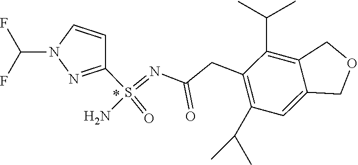 [SMILES: CC(C)c1cc2c(c(C(C)C)c1CC(=O)N=S(N)(=O)c1ccn(C(F)F)n1)COC2] |
| 1328b | 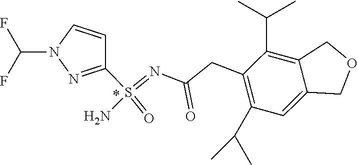 [SMILES: CC(C)c1cc2c(c(C(C)C)c1CC(=O)N=S(N)(=O)c1ccn(C(F)F)n1)COC2] |
| 1329 | 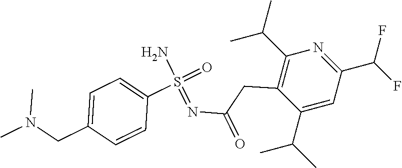 [SMILES: CC(C)c1cc(C(F)F)nc(C(C)C)c1CC(=O)N=S(N)(=O)c1ccc(CN(C)C)cc1] |
| 1329a | 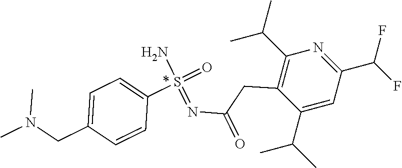 [SMILES: CC(C)c1cc(C(F)F)nc(C(C)C)c1CC(=O)N=S(N)(=O)c1ccc(CN(C)C)cc1] |
| 1329b | 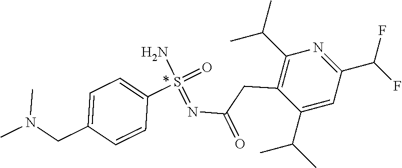 [SMILES: CC(C)c1cc(C(F)F)nc(C(C)C)c1CC(=O)N=S(N)(=O)c1ccc(CN(C)C)cc1] |
| 1330 | 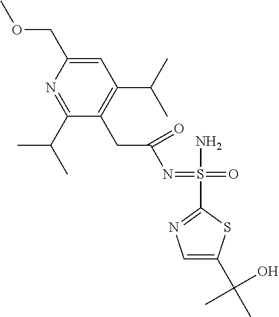 [SMILES: COCc1cc(C(C)C)c(CC(=O)N=S(N)(=O)c2ncc(C(C)(C)O)s2)c(C(C)C)n1] |
| 1330a | 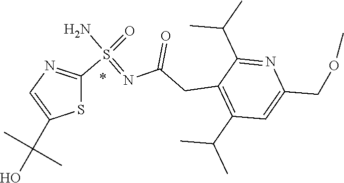 [SMILES: COCc1cc(C(C)C)c(CC(=O)N=S(N)(=O)c2ncc(C(C)(C)O)s2)c(C(C)C)n1] |
| 1330b | 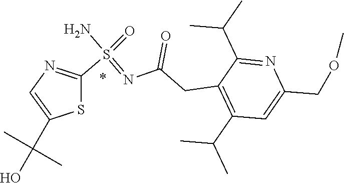 [SMILES: COCc1cc(C(C)C)c(CC(=O)N=S(N)(=O)c2ncc(C(C)(C)O)s2)c(C(C)C)n1] |
| 1331 | 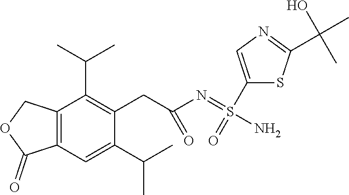 [SMILES: CC(C)c1cc2c(c(C(C)C)c1CC(=O)N=S(N)(=O)c1cnc(C(C)(C)O)s1)COC2=O] |
| 1331a | 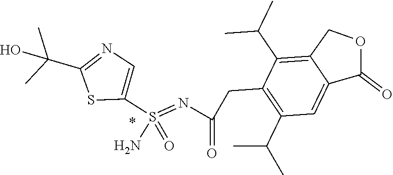 [SMILES: CC(C)c1cc2c(c(C(C)C)c1CC(=O)N=S(N)(=O)c1cnc(C(C)(C)O)s1)COC2=O] |
| 1331b | 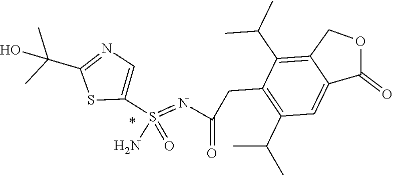 [SMILES: CC(C)c1cc2c(c(C(C)C)c1CC(=O)N=S(N)(=O)c1cnc(C(C)(C)O)s1)COC2=O] |
| 1332 | 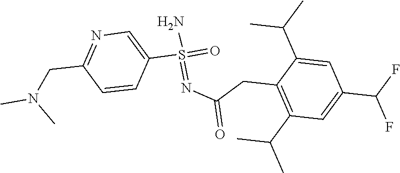 [SMILES: CC(C)c1cc(C(F)F)cc(C(C)C)c1CC(=O)N=S(N)(=O)c1ccc(CN(C)C)nc1] |
| 1332a | 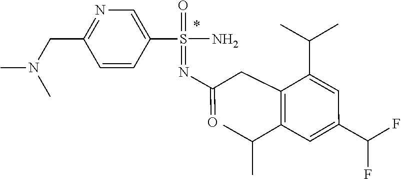 [SMILES: CC(C)c1cc(C(F)F)cc(C(C)C)c1CC(=O)N=S(N)(=O)c1ccc(CN(C)C)nc1] |
| 1332b | 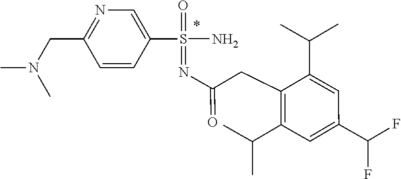 [SMILES: CC(C)c1cc(C(F)F)cc(C(C)C)c1CC(=O)N=S(N)(=O)c1ccc(CN(C)C)nc1] |
| 1333 | 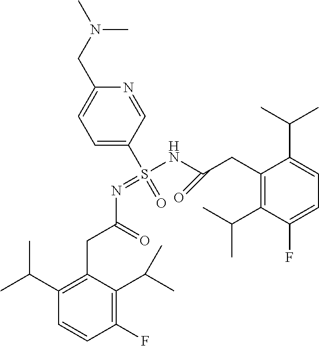 [SMILES: CC(C)c1ccc(F)c(C(C)C)c1CC(=O)N=S(=O)(NC(=O)Cc1c(C(C)C)ccc(F)c1C(C)C)c1ccc(CN(C)C)nc1] |
| 1334 | 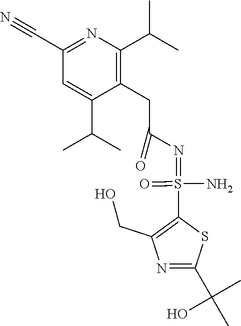 [SMILES: CC(C)c1cc(C#N)nc(C(C)C)c1CC(=O)N=S(N)(=O)c1sc(C(C)(C)O)nc1CO] |
| 1335 | 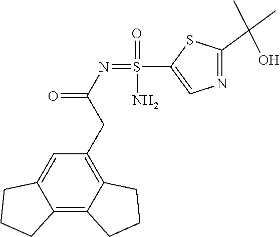 [SMILES: CC(C)(O)c1ncc(S(N)(=O)=NC(=O)Cc2cc3c(c4c2CCC4)CCC3)s1] |
| 1335a | 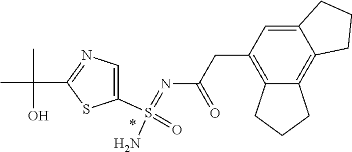 [SMILES: CC(C)(O)c1ncc(S(N)(=O)=NC(=O)Cc2cc3c(c4c2CCC4)CCC3)s1] |
| 1335b | 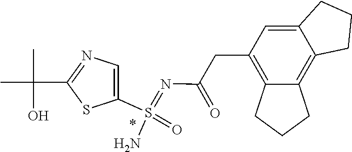 [SMILES: CC(C)(O)c1ncc(S(N)(=O)=NC(=O)Cc2cc3c(c4c2CCC4)CCC3)s1] |
| 1336a | 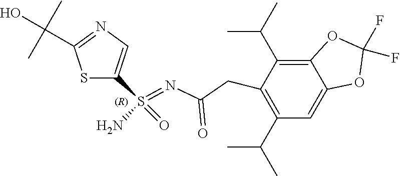 |
| 1337a | 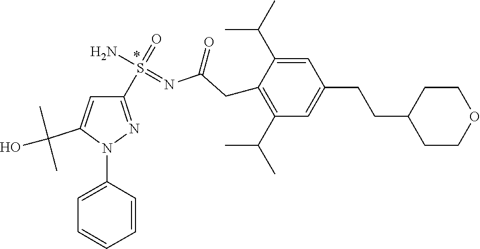 [SMILES: CC(C)c1cc(CCC2CCOCC2)cc(C(C)C)c1CC(=O)N=S(N)(=O)c1cc(C(C)(C)O)n(-c2ccccc2)n1] |
| 1337b | 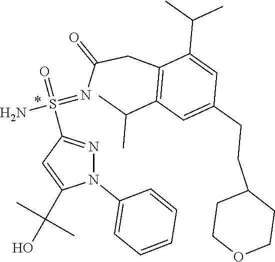 [SMILES: CC(C)c1cc(CCC2CCOCC2)cc(C(C)C)c1CC(=O)N=S(N)(=O)c1cc(C(C)(C)O)n(-c2ccccc2)n1] |
| 1338 | 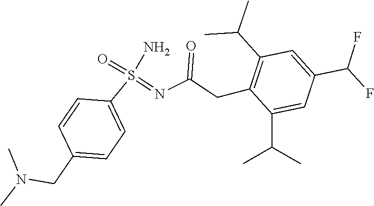 [SMILES: CC(C)c1cc(C(F)F)cc(C(C)C)c1CC(=O)N=S(N)(=O)c1ccc(CN(C)C)cc1] |
| 1338a | 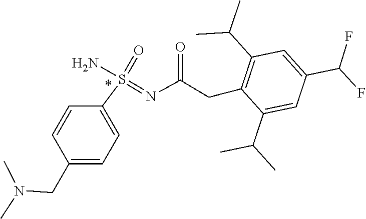 [SMILES: CC(C)c1cc(C(F)F)cc(C(C)C)c1CC(=O)N=S(N)(=O)c1ccc(CN(C)C)cc1] |
| 1338b | 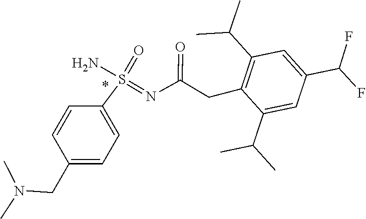 [SMILES: CC(C)c1cc(C(F)F)cc(C(C)C)c1CC(=O)N=S(N)(=O)c1ccc(CN(C)C)cc1] |
| 1339 | 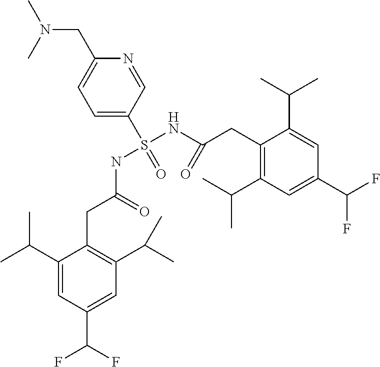 |
| 1340a | 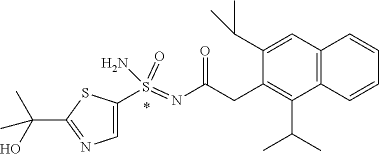 [SMILES: CC(C)c1cc2ccccc2c(C(C)C)c1CC(=O)N=S(N)(=O)c1cnc(C(C)(C)O)s1] |
| 1341 | 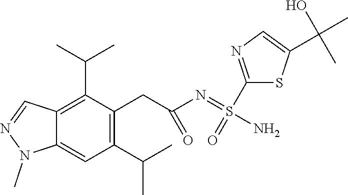 [SMILES: CC(C)c1cc2c(cnn2C)c(C(C)C)c1CC(=O)N=S(N)(=O)c1ncc(C(C)(C)O)s1] |
| 1342a | 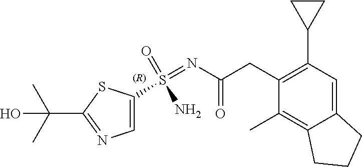 |
| 1343a | 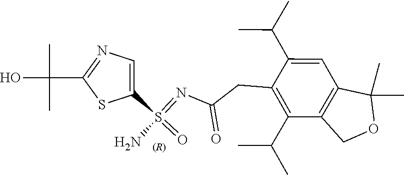 |
| 1344a | 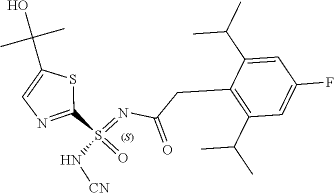 |
| 1344b | 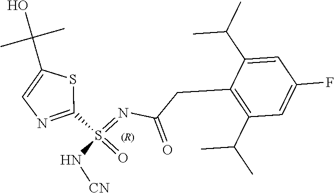 |
| 1345a | 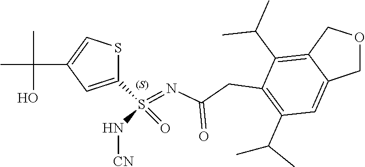 |
| 1345b | 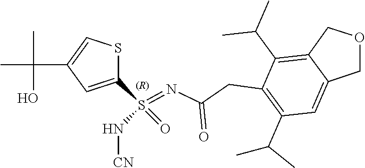 |
| 1346a | 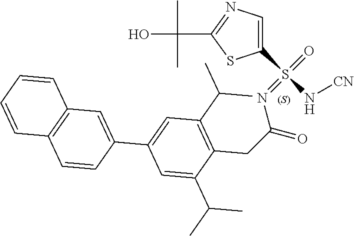 |
| 1346b | 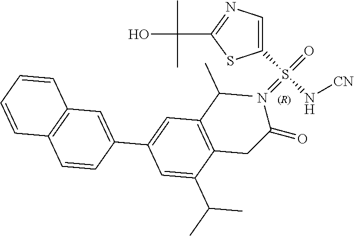 |

or a pharmaceutically acceptable salt thereof.

In some embodiments, provided herein is a compound that is selected from the group consisting of the compounds in Table 1-7:

TABLE 1-7

| Cmpd | | Cmpd | |
|---|---|---|---|
| # | Structure | # | Structure |
| 1401 | 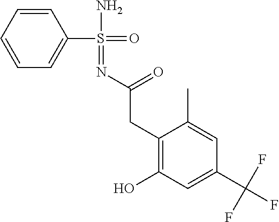 [SMILES: Cc1cc(C(F)(F)F)cc(O)c1CC(=O)N=S(N)(=O)c1ccccc1] | 1402a | 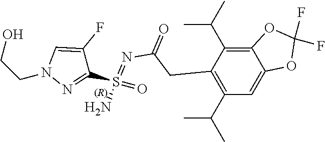 |
| 1402b | 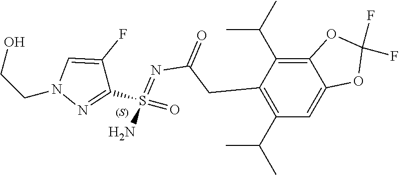 | 1403a | 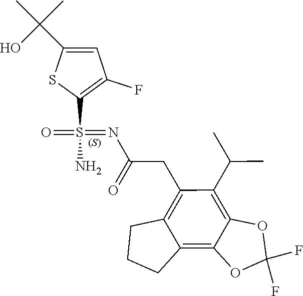 |
| 1403b | 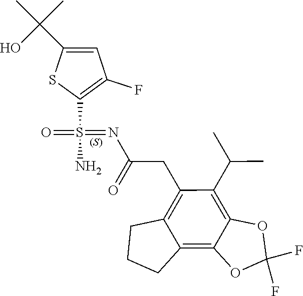 | 1404 | 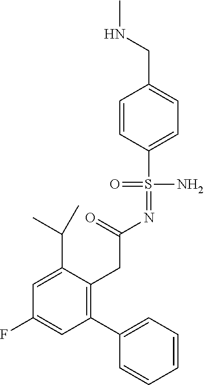 [SMILES: CNCc1ccc(S(N)(=O)=NC(=O)Cc2c(-c3ccccc3)cc(F)cc2C(C)C)cc1] |
| 1405 | 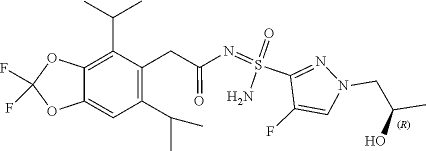 | 1406a | 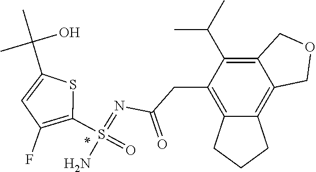 [SMILES: CC(C)c1c2c(c3c(c1CC(=O)N=S(N)(=O)c1sc(C(C)(C)O)cc1F)CCC3)COC2] |
| 1406b | 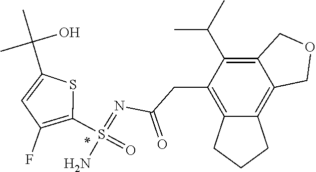 [SMILES: CC(C)c1c2c(c3c(c1CC(=O)N=S(N)(=O)c1sc(C(C)(C)O)cc1F)CCC3)COC2] | 1407a | 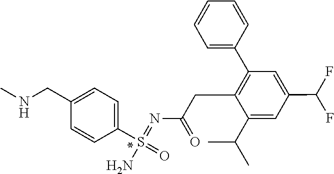 [SMILES: CNCc1ccc(S(N)(=O)=NC(=O)Cc2c(-c3ccccc3)cc(C(F)F)cc2C(C)C)cc1] |
| 1407b | 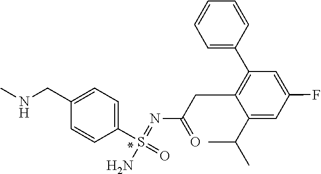 [SMILES: CNCc1ccc(S(N)(=O)=NC(=O)Cc2c(-c3ccccc3)cc(F)cc2C(C)C)cc1] | 1408a | 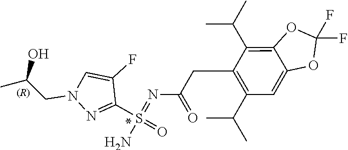 |
| 1408b | 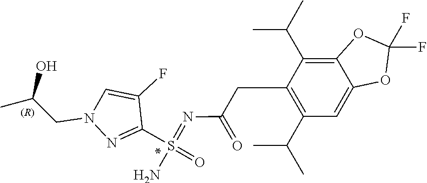 | | |

or a pharmaceutically acceptable salt thereof.

In any of the embodiments described herein, the compound of Formula AA is not one of the following:

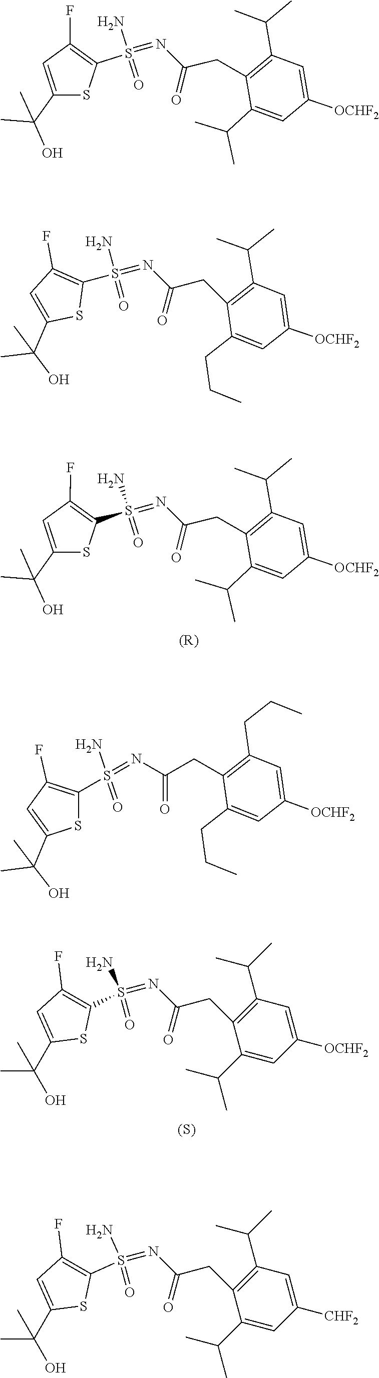

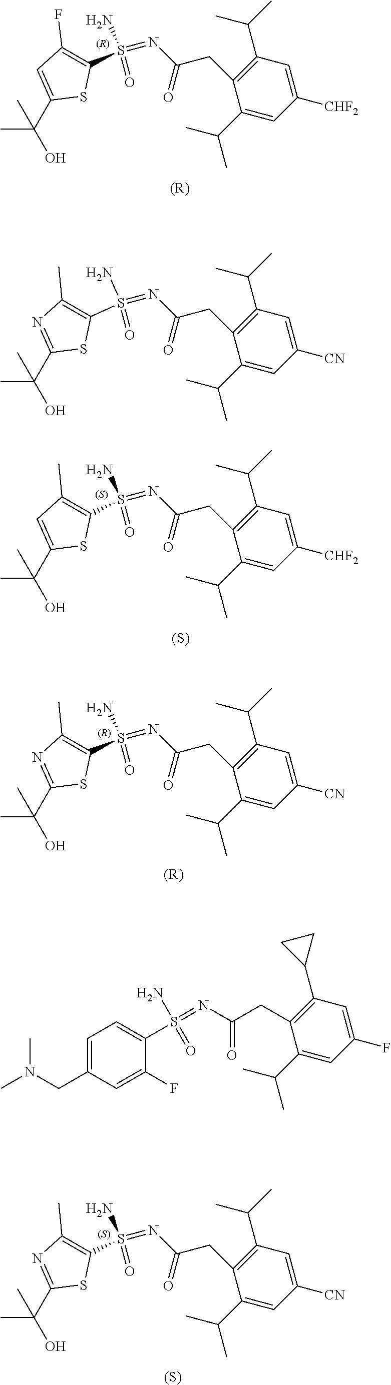

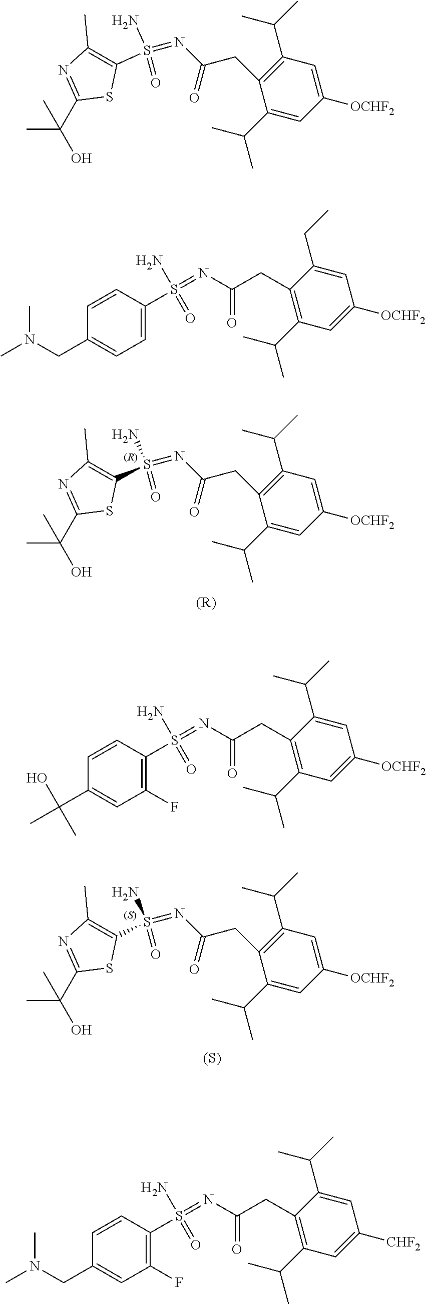

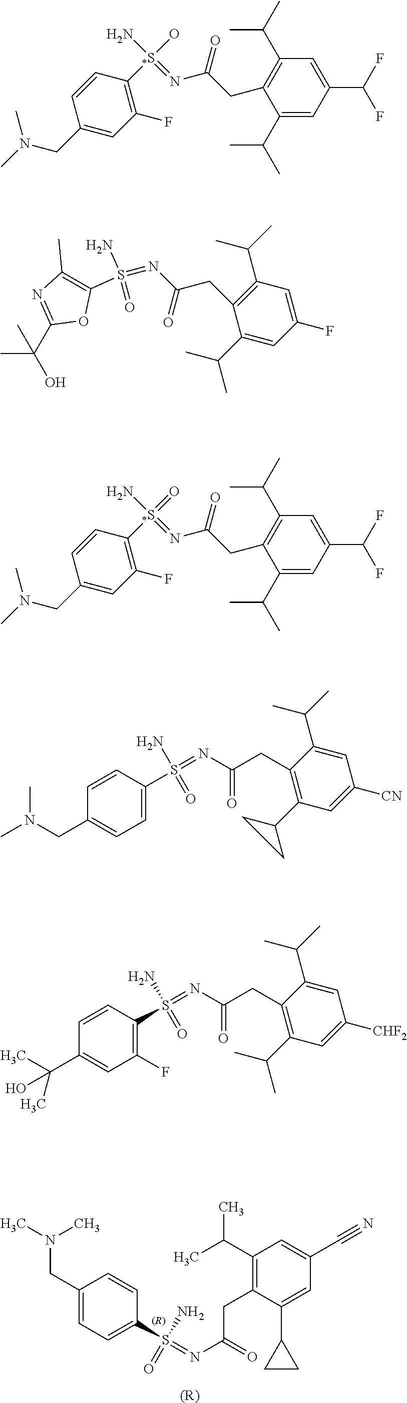

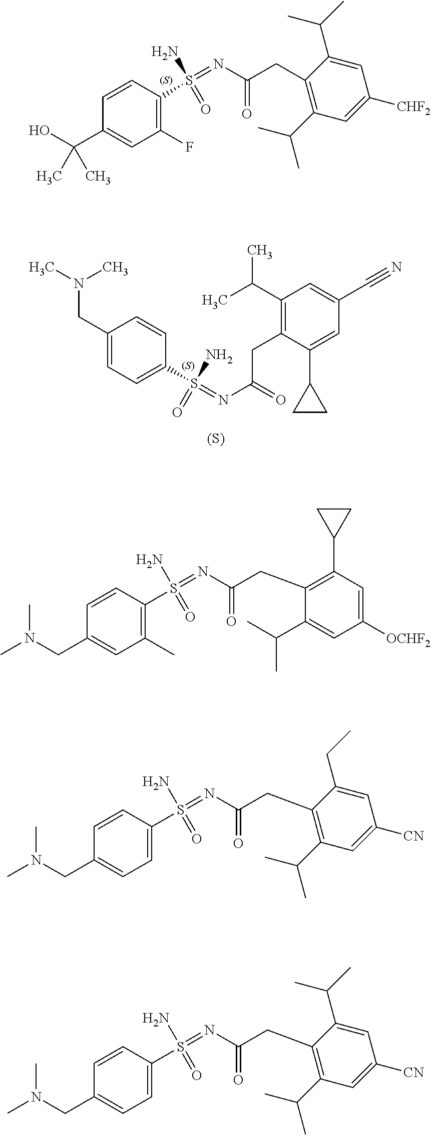

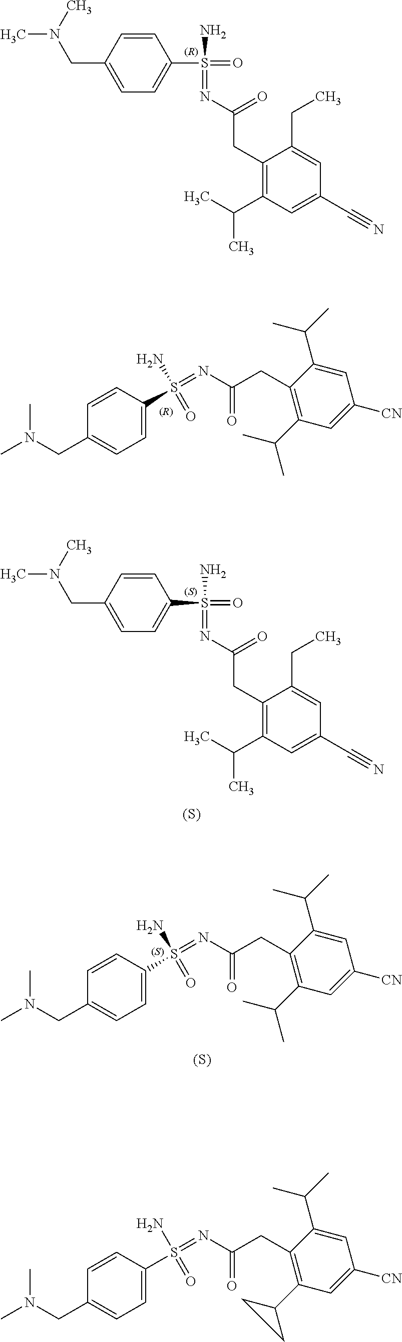

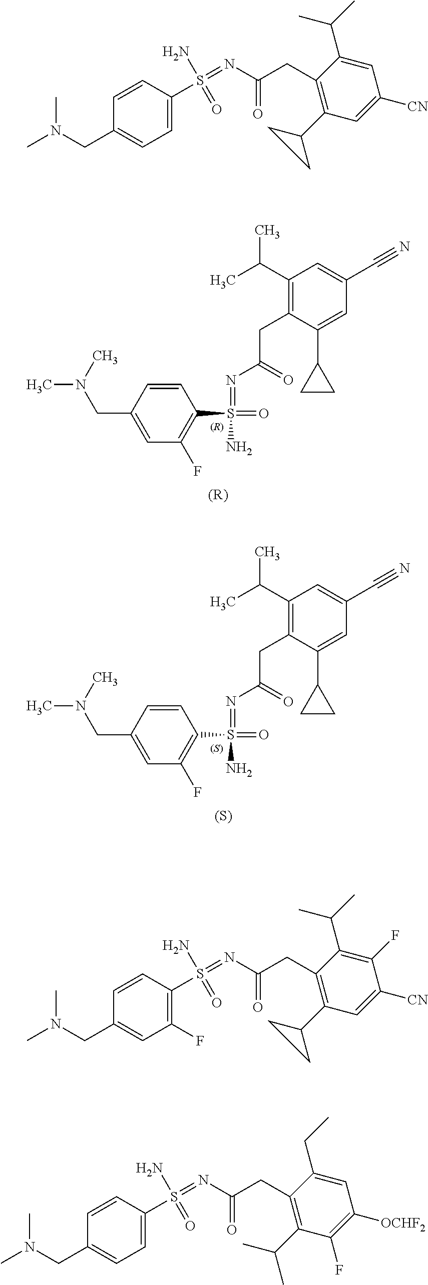

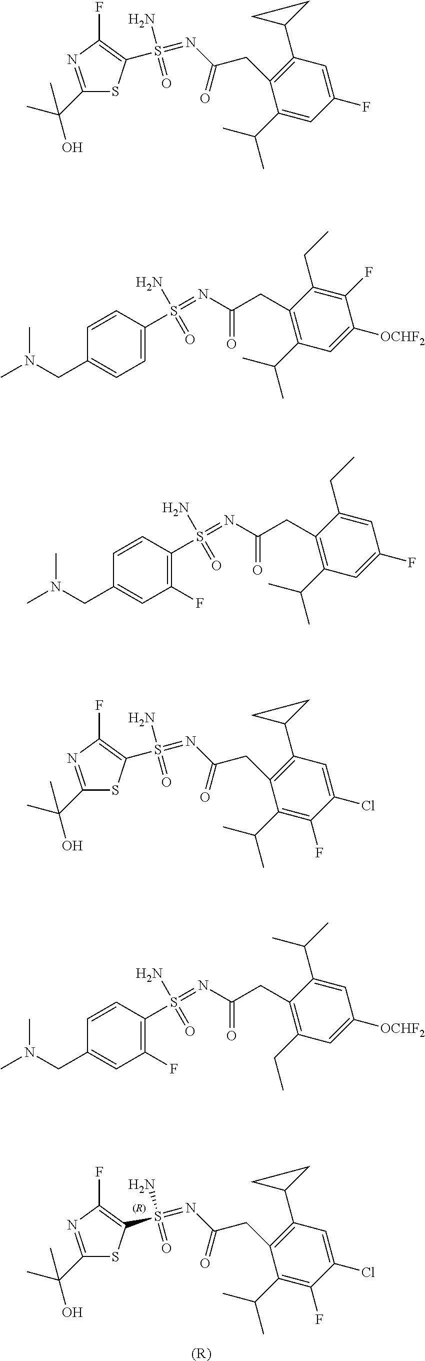

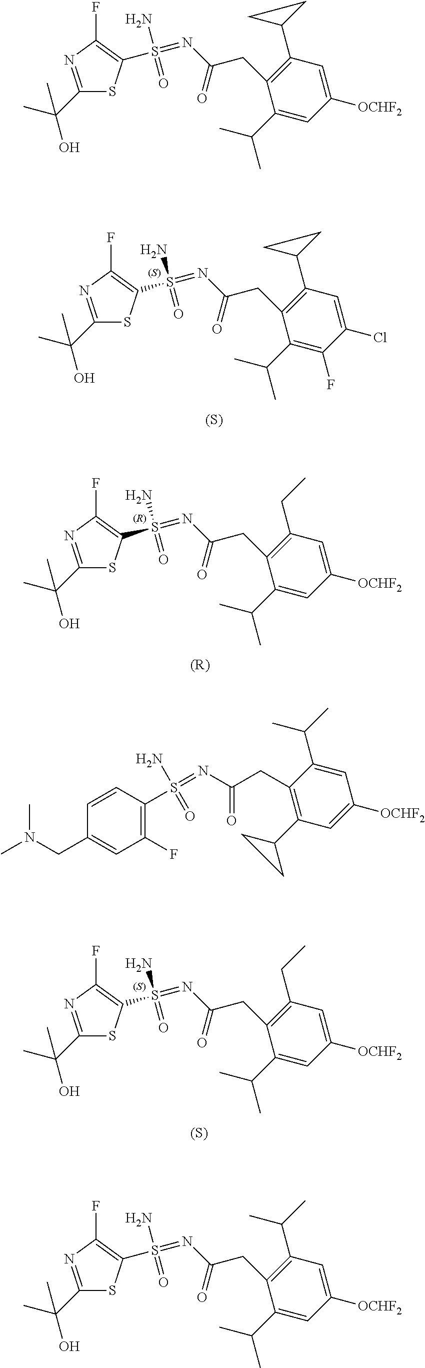

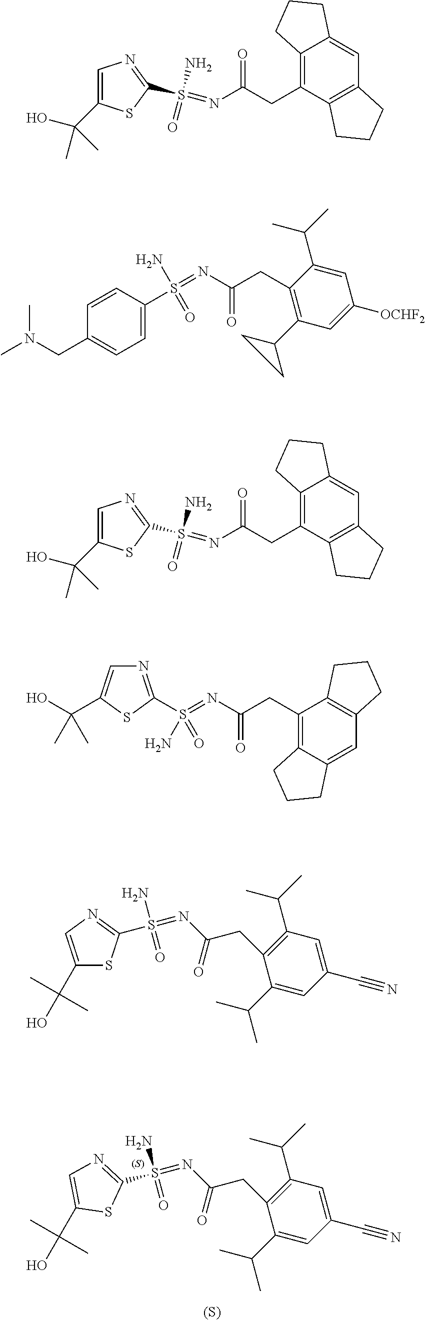

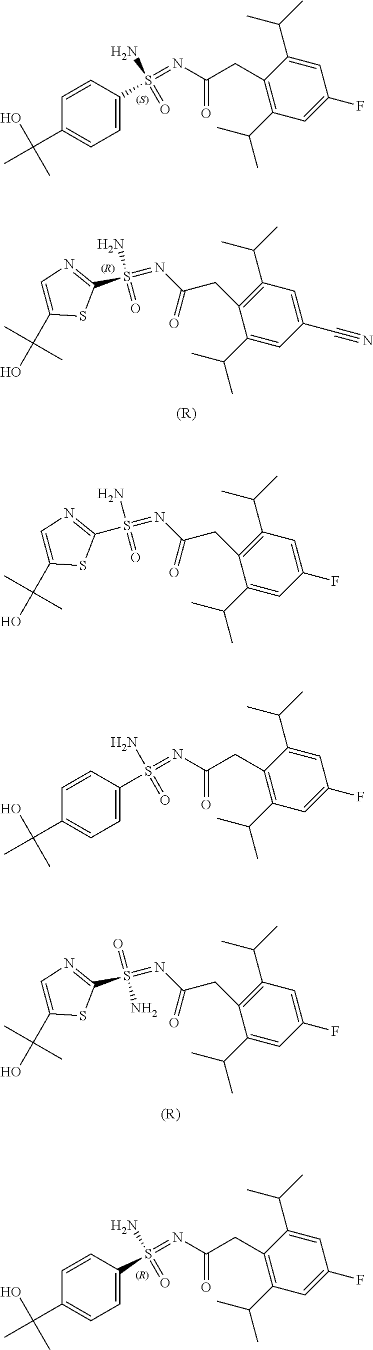

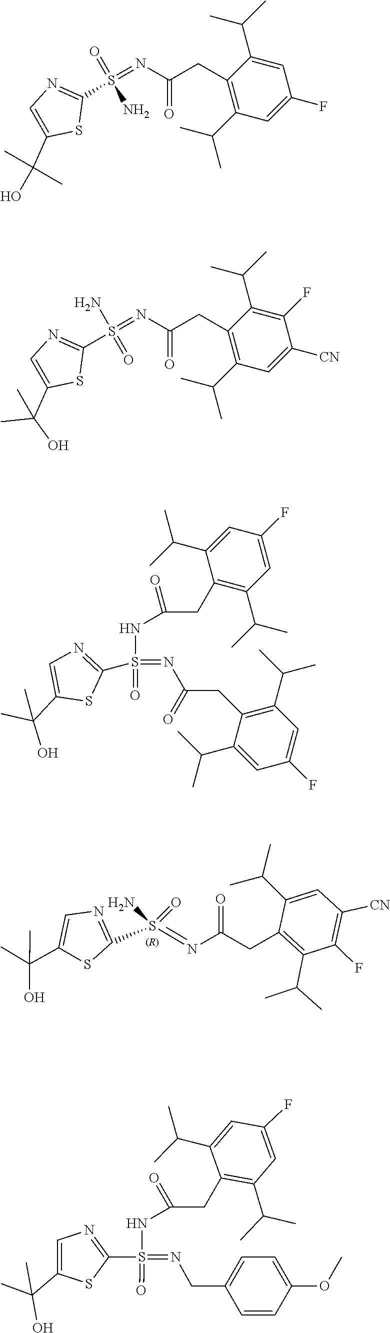

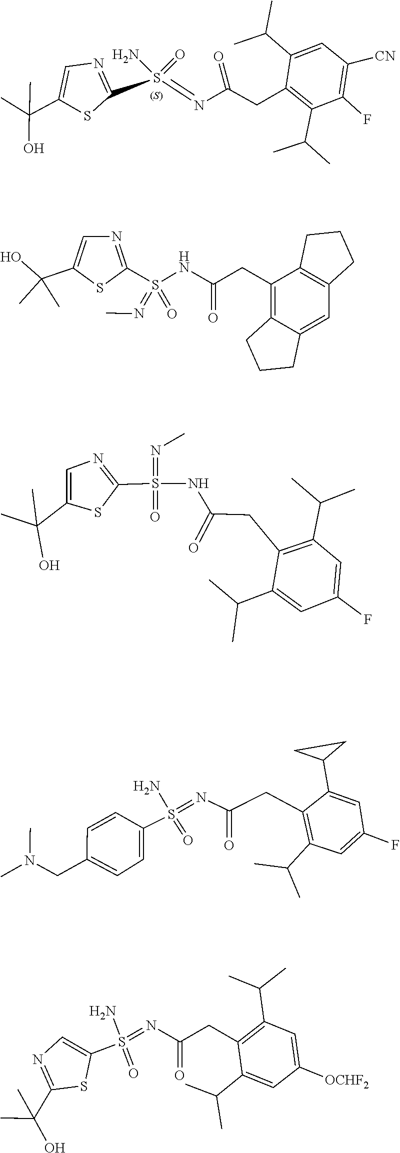

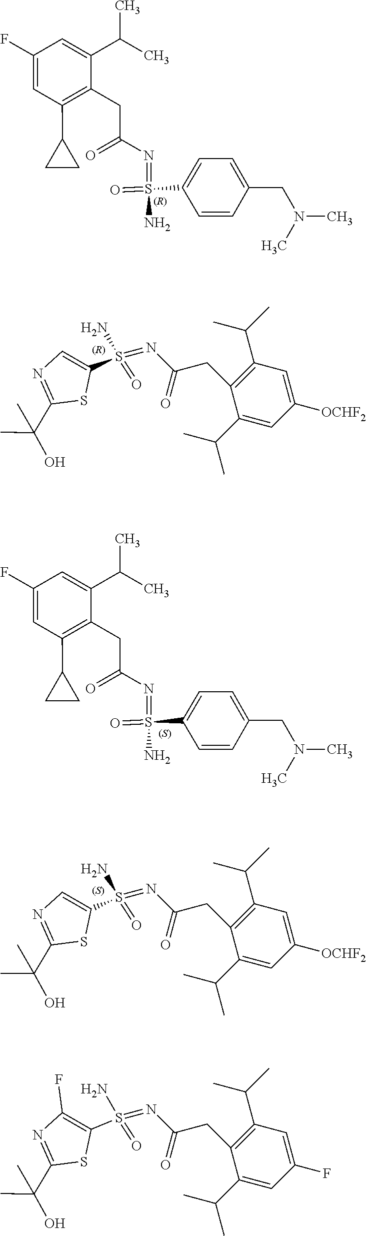

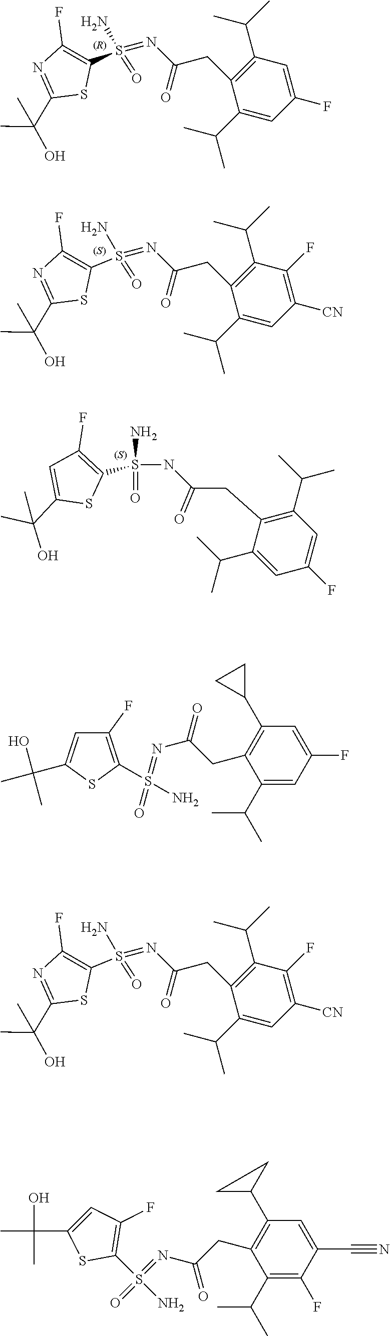

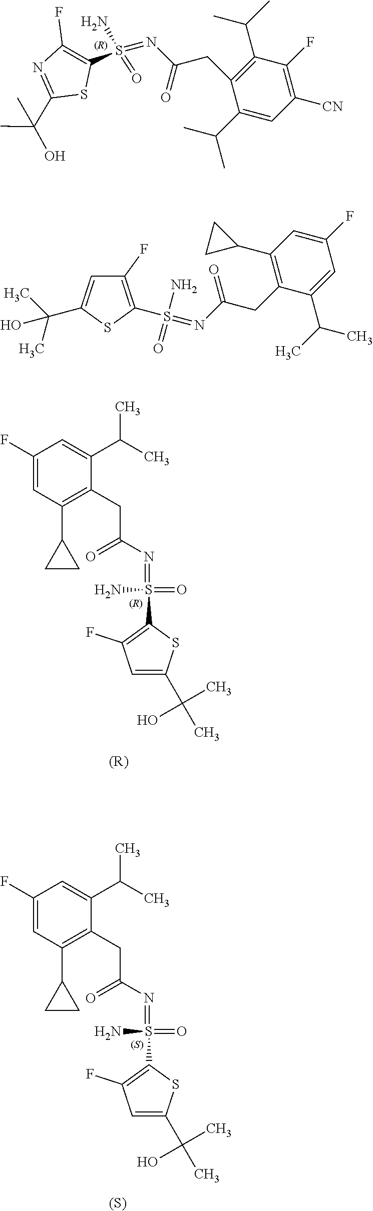

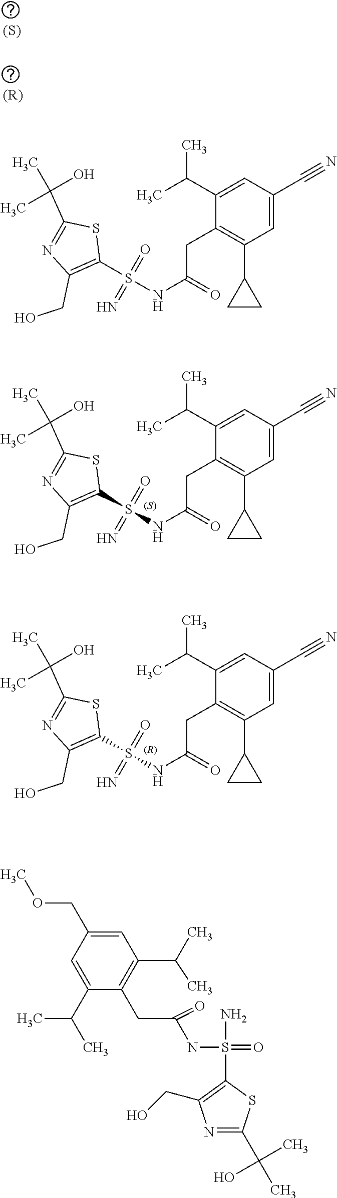

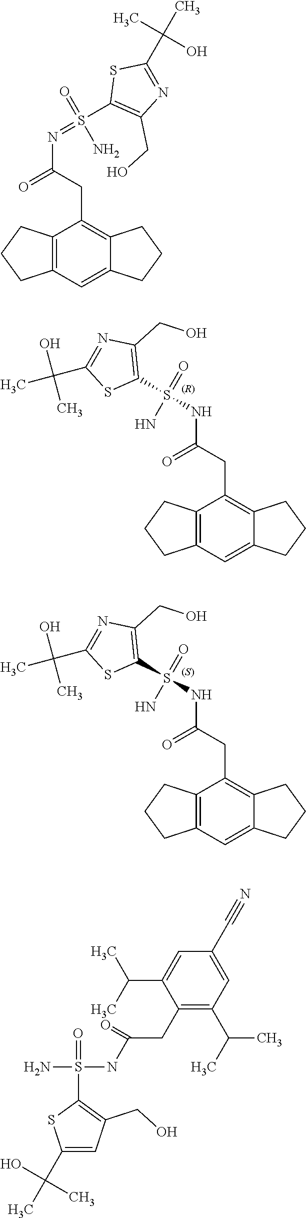

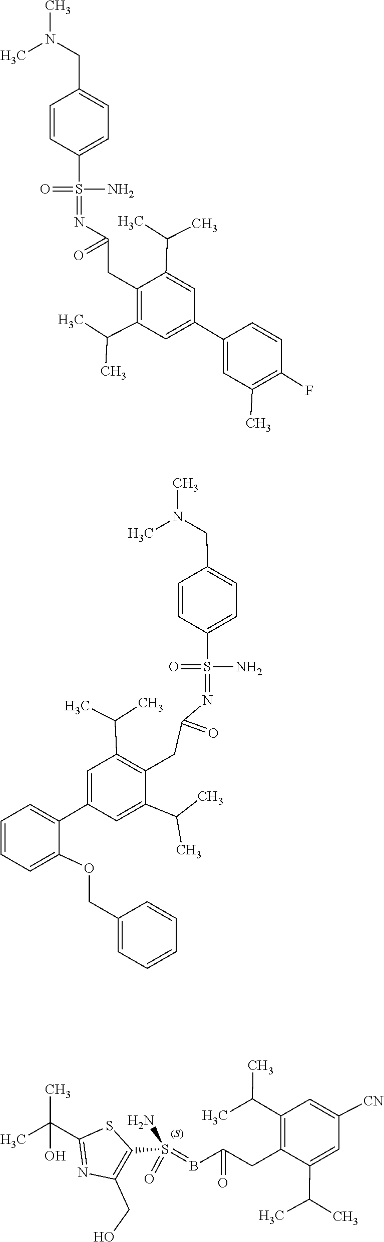

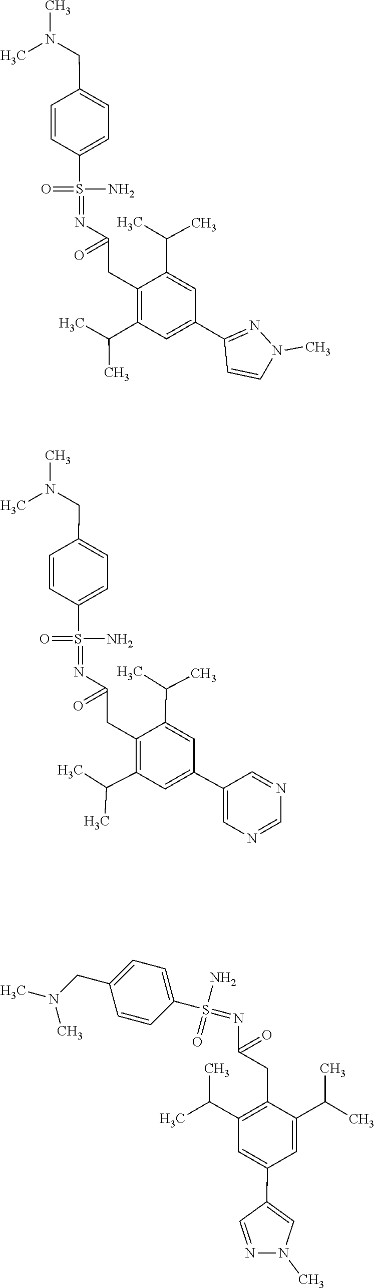

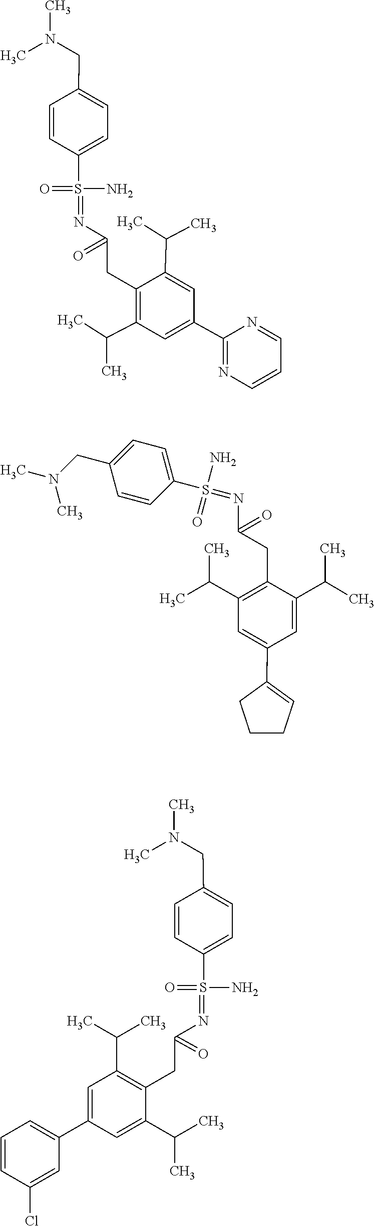 [SMILES: CC(C)c1cc(-c2ncccn2)cc(C(C)C)c1CC(=O)N=S(N)(=O)c1ccc(CN(C)C)cc1 ; CC(C)c1cc(C2=CCCC2)cc(C(C)C)c1CC(=O)N=S(N)(=O)c1ccc(CN(C)C)cc1 ; CC(C)c1cc(-c2cccc(Cl)c2)cc(C(C)C)c1CC(=O)N=S(N)(=O)c1ccc(CN(C)C)cc1]

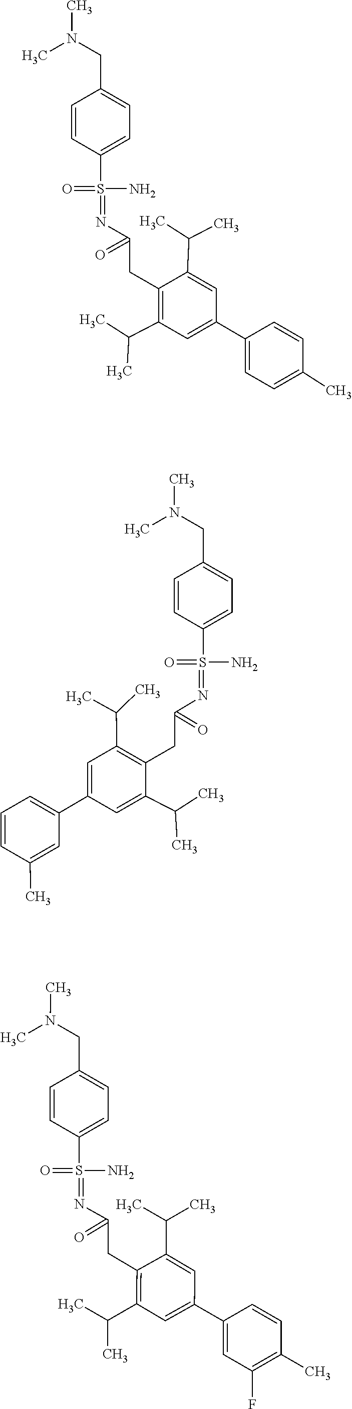

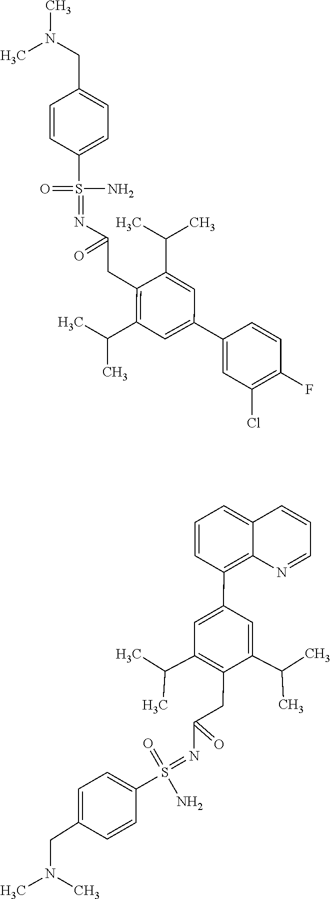 [SMILES: CC(C)c1cc(-c2ccc(F)c(Cl)c2)cc(C(C)C)c1CC(=O)N=S(N)(=O)c1ccc(CN(C)C)cc1 ; CC(C)c1cc(-c2cccc3cccnc23)cc(C(C)C)c1CC(=O)N=S(N)(=O)c1ccc(CN(C)C)cc1]

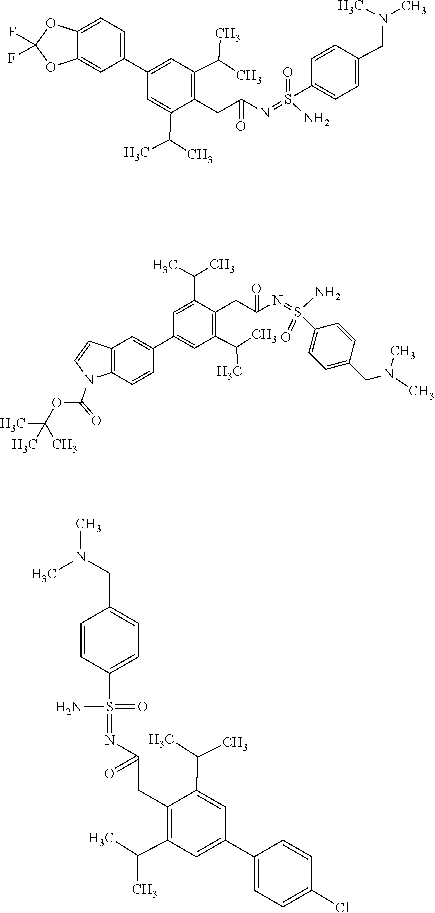

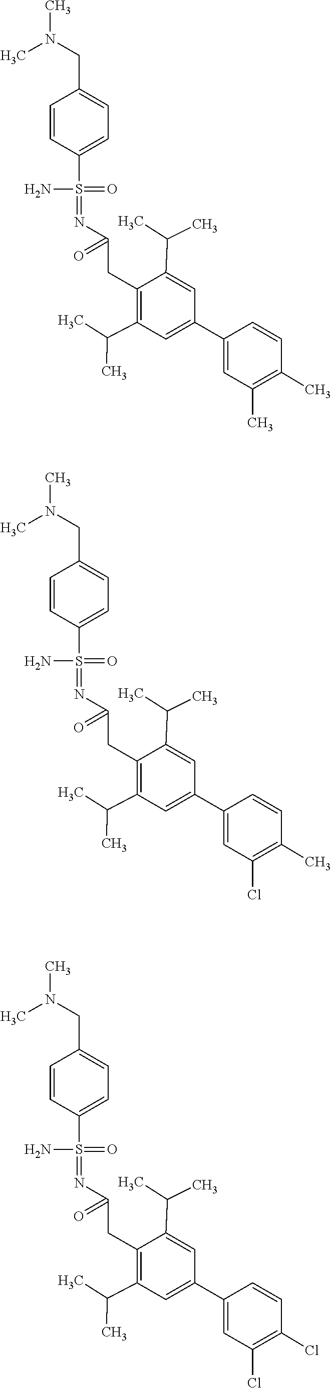

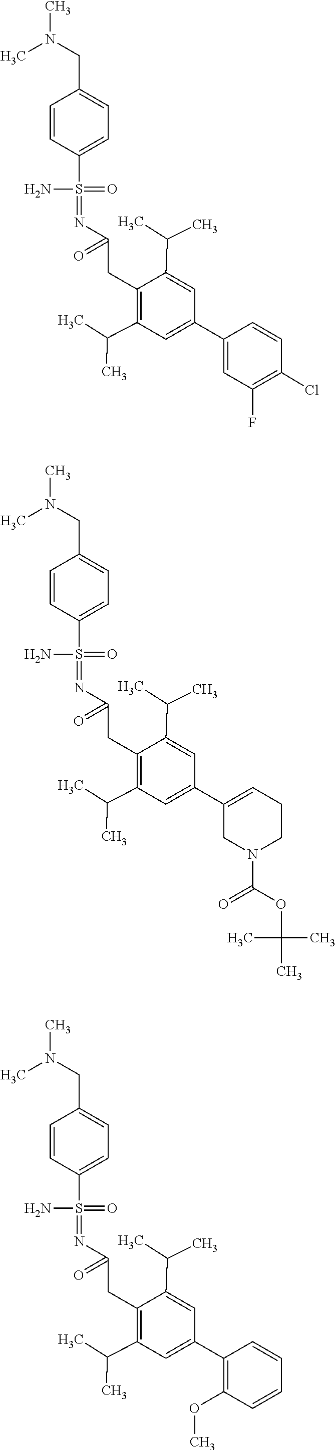

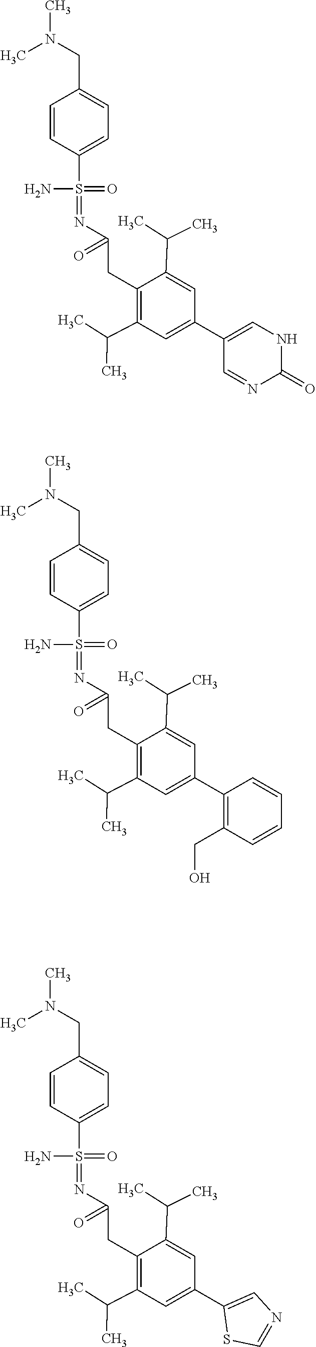 [SMILES: CC(C)c1cc(-c2cnc(=O)[nH]c2)cc(C(C)C)c1CC(=O)N=S(N)(=O)c1ccc(CN(C)C)cc1 ; CC(C)c1cc(-c2ccccc2CO)cc(C(C)C)c1CC(=O)N=S(N)(=O)c1ccc(CN(C)C)cc1 ; CC(C)c1cc(-c2cncs2)cc(C(C)C)c1CC(=O)N=S(N)(=O)c1ccc(CN(C)C)cc1]

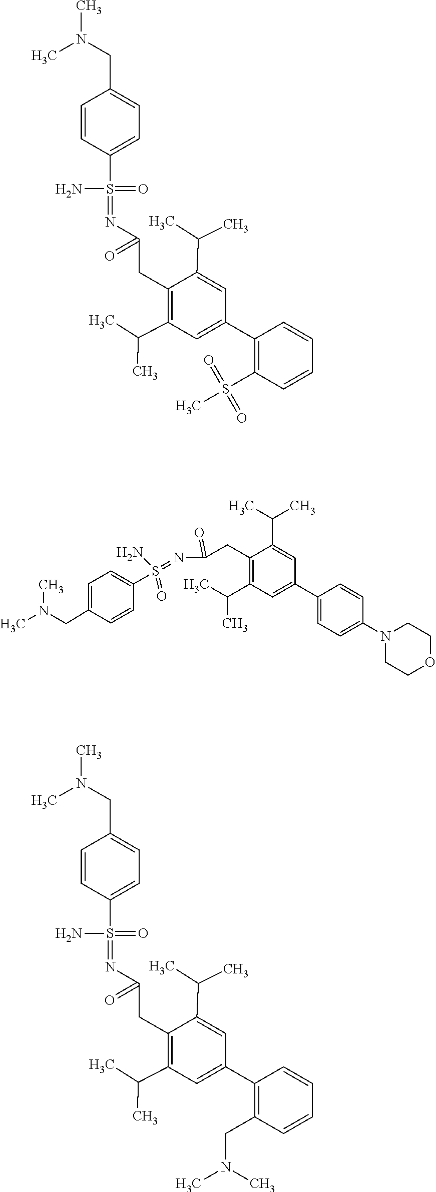 [SMILES: CC(C)c1cc(-c2ccccc2S(C)(=O)=O)cc(C(C)C)c1CC(=O)N=S(N)(=O)c1ccc(CN(C)C)cc1 ; CC(C)c1cc(-c2ccc(N3CCOCC3)cc2)cc(C(C)C)c1CC(=O)N=S(N)(=O)c1ccc(CN(C)C)cc1 ; CC(C)c1cc(-c2ccccc2CN(C)C)cc(C(C)C)c1CC(=O)N=S(N)(=O)c1ccc(CN(C)C)cc1]

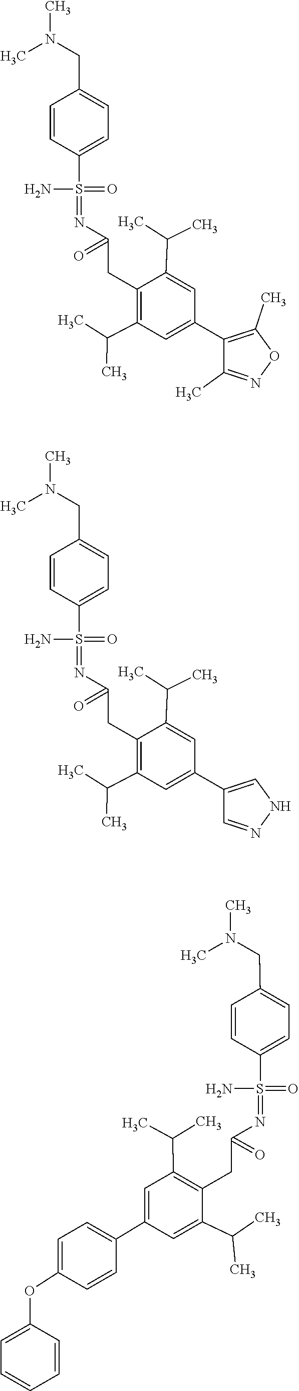 [SMILES: Cc1noc(C)c1-c1cc(C(C)C)c(CC(=O)N=S(N)(=O)c2ccc(CN(C)C)cc2)c(C(C)C)c1 ; CC(C)c1cc(-c2cn[nH]c2)cc(C(C)C)c1CC(=O)N=S(N)(=O)c1ccc(CN(C)C)cc1 ; CC(C)c1cc(-c2ccc(Oc3ccccc3)cc2)cc(C(C)C)c1CC(=O)N=S(N)(=O)c1ccc(CN(C)C)cc1]

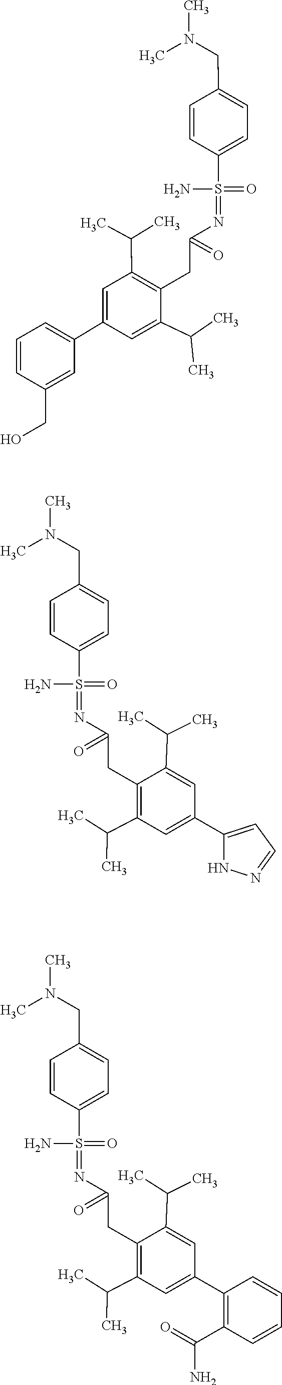 [SMILES: CC(C)c1cc(-c2cccc(CO)c2)cc(C(C)C)c1CC(=O)N=S(N)(=O)c1ccc(CN(C)C)cc1 ; CC(C)c1cc(-c2ccn[nH]2)cc(C(C)C)c1CC(=O)N=S(N)(=O)c1ccc(CN(C)C)cc1 ; CC(C)c1cc(-c2ccccc2C(N)=O)cc(C(C)C)c1CC(=O)N=S(N)(=O)c1ccc(CN(C)C)cc1]

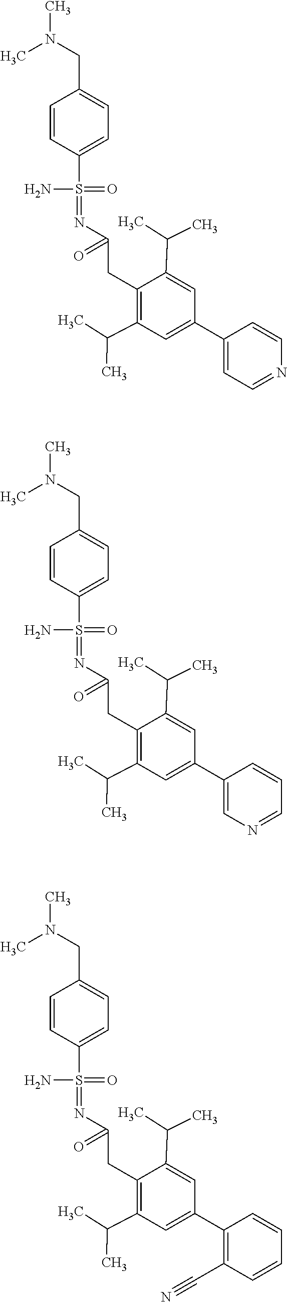 [SMILES: CC(C)c1cc(-c2ccncc2)cc(C(C)C)c1CC(=O)N=S(N)(=O)c1ccc(CN(C)C)cc1 ; CC(C)c1cc(-c2cccnc2)cc(C(C)C)c1CC(=O)N=S(N)(=O)c1ccc(CN(C)C)cc1 ; CC(C)c1cc(-c2ccccc2C#N)cc(C(C)C)c1CC(=O)N=S(N)(=O)c1ccc(CN(C)C)cc1]

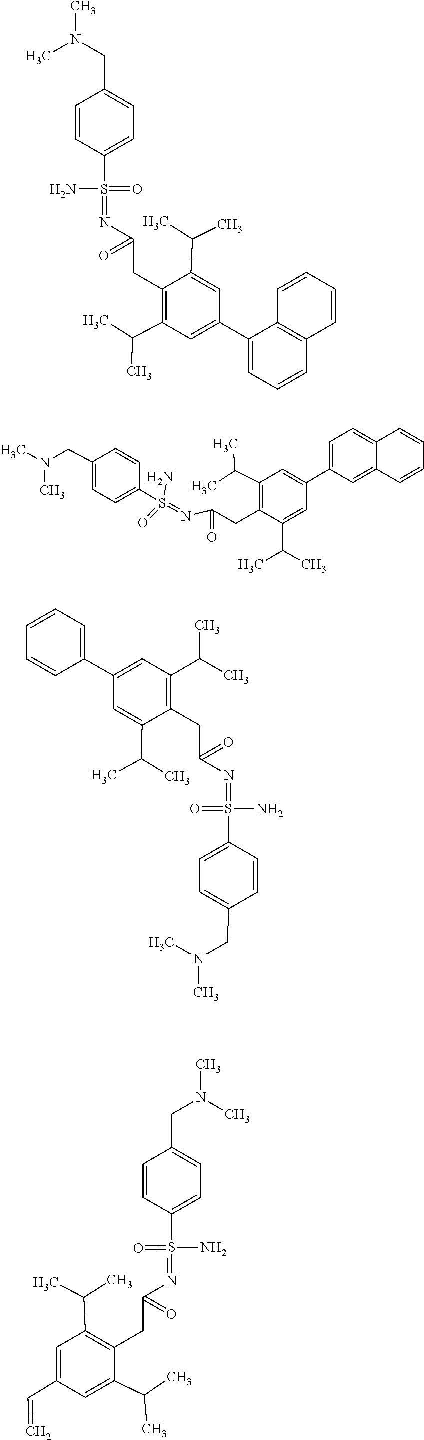 [SMILES: CC(C)c1cc(-c2cccc3ccccc23)cc(C(C)C)c1CC(=O)N=S(N)(=O)c1ccc(CN(C)C)cc1 ; CC(C)c1cc(-c2ccc3ccccc3c2)cc(C(C)C)c1CC(=O)N=S(N)(=O)c1ccc(CN(C)C)cc1 ; CC(C)c1cc(-c2ccccc2)cc(C(C)C)c1CC(=O)N=S(N)(=O)c1ccc(CN(C)C)cc1 ; C=Cc1cc(C(C)C)c(CC(=O)N=S(N)(=O)c2ccc(CN(C)C)cc2)c(C(C)C)c1]

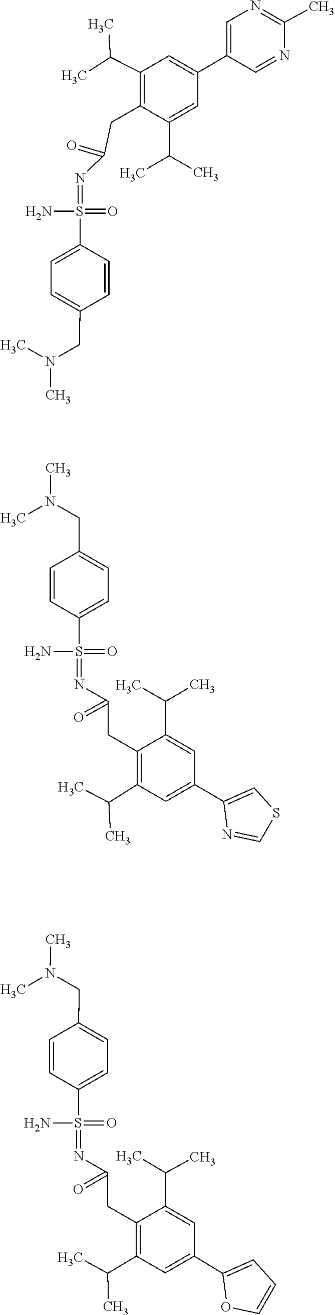

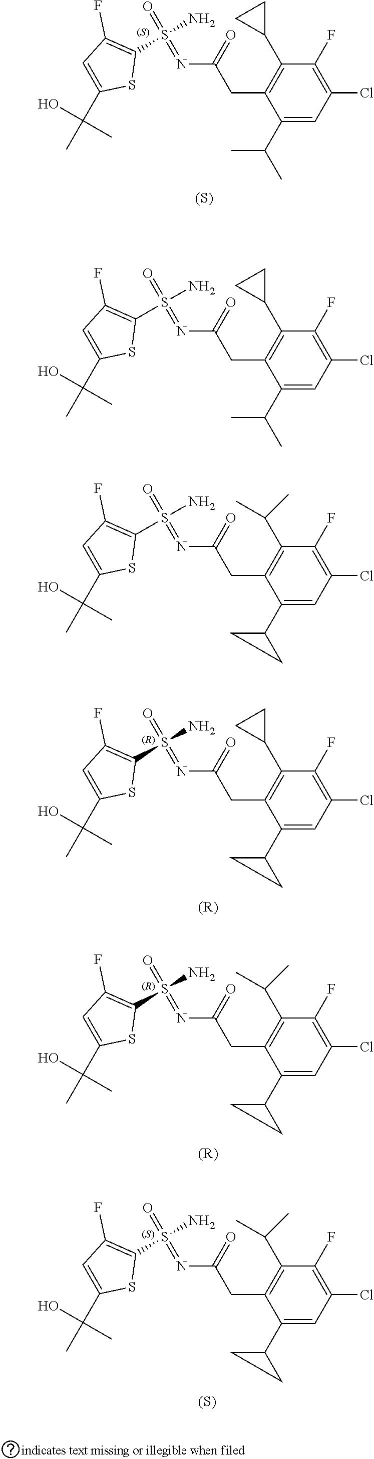

or a pharmaceutically acceptable salt thereof.

In any of the embodiments described herein, the compound of Formula AA is not one of the following:

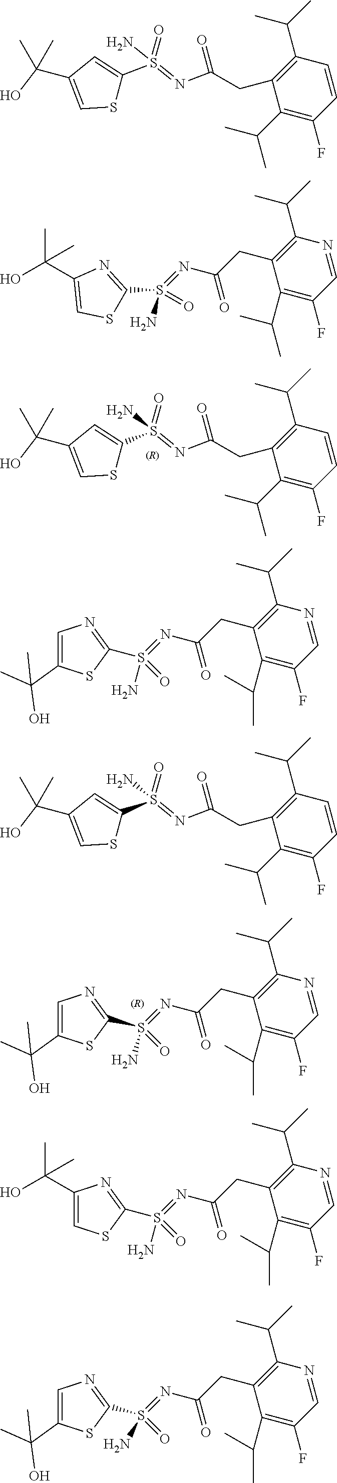

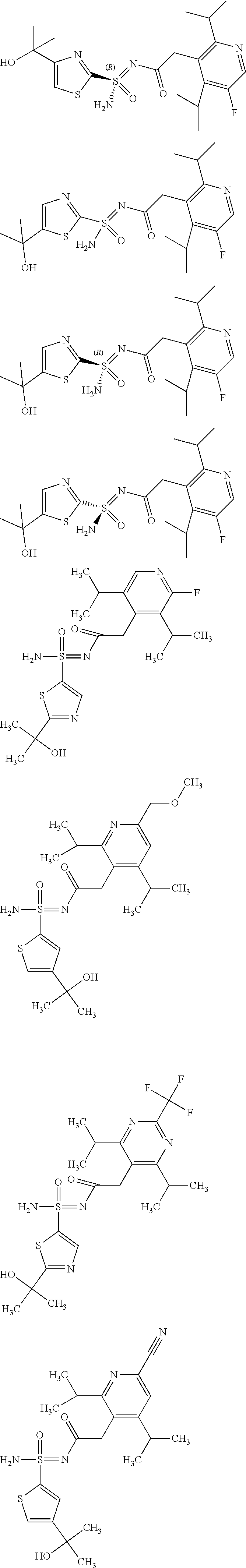

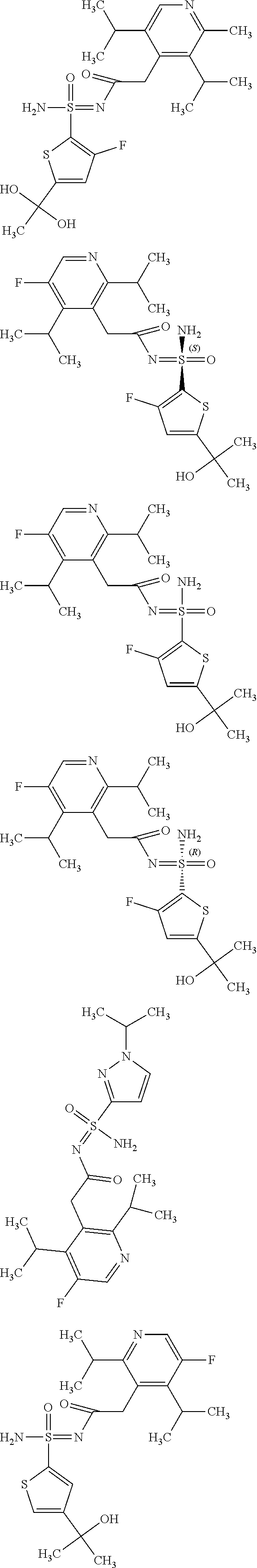

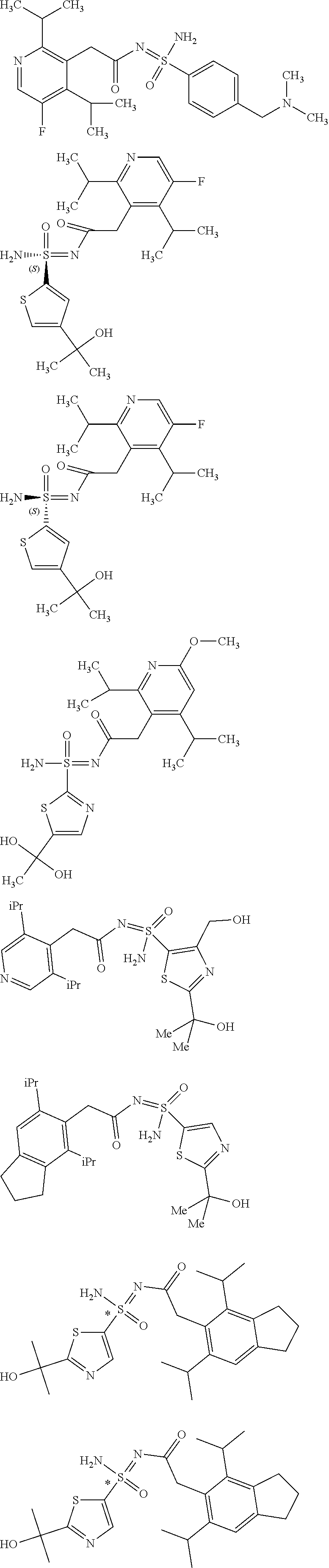

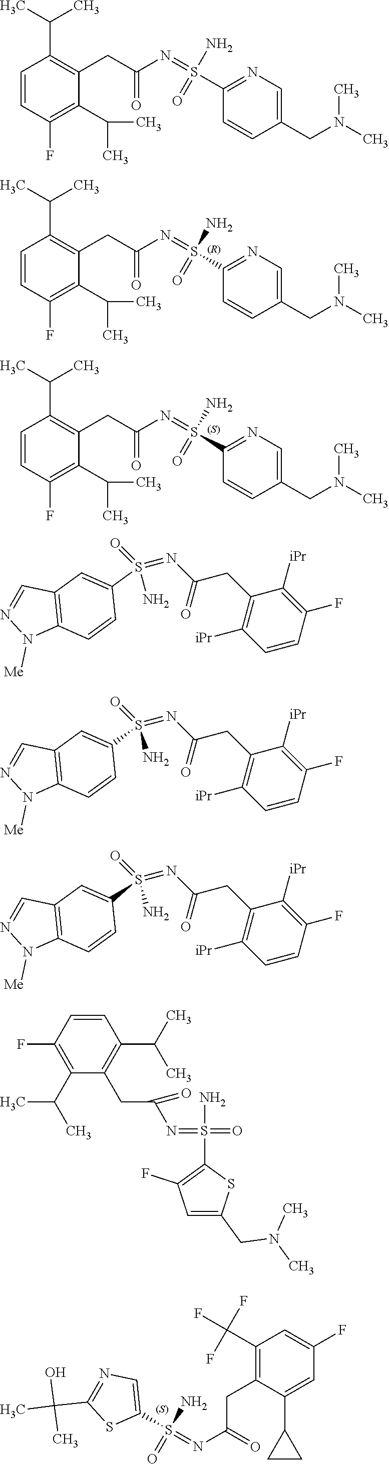

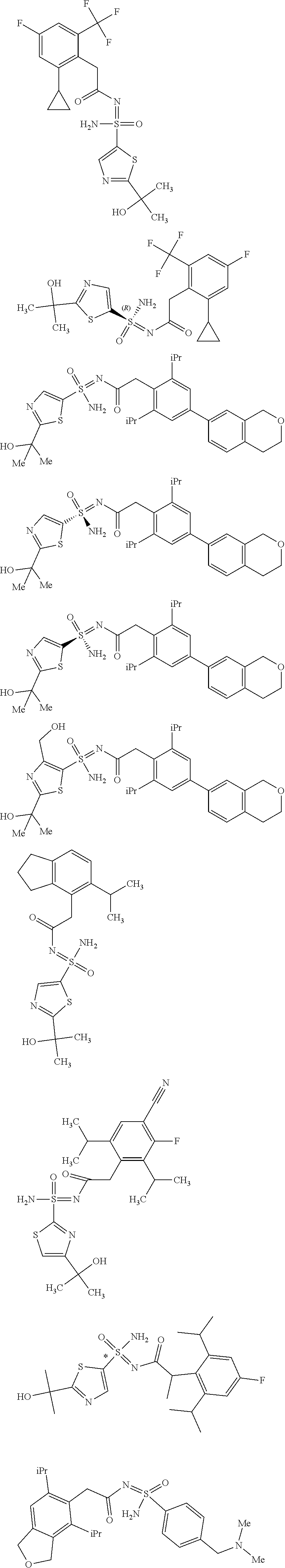

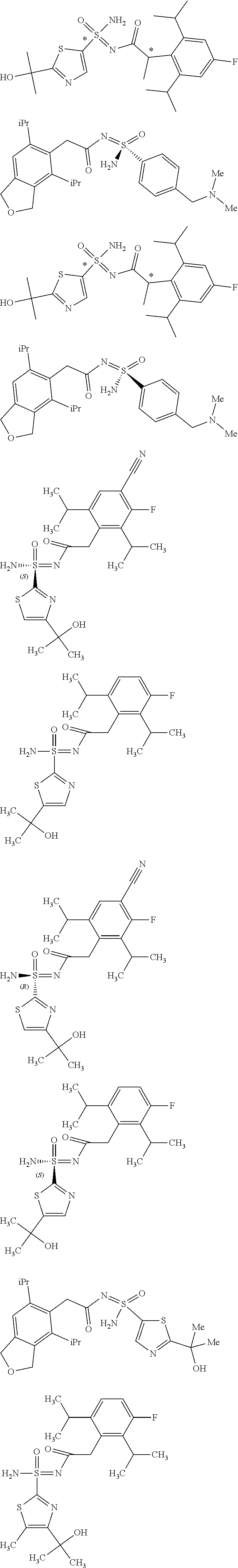

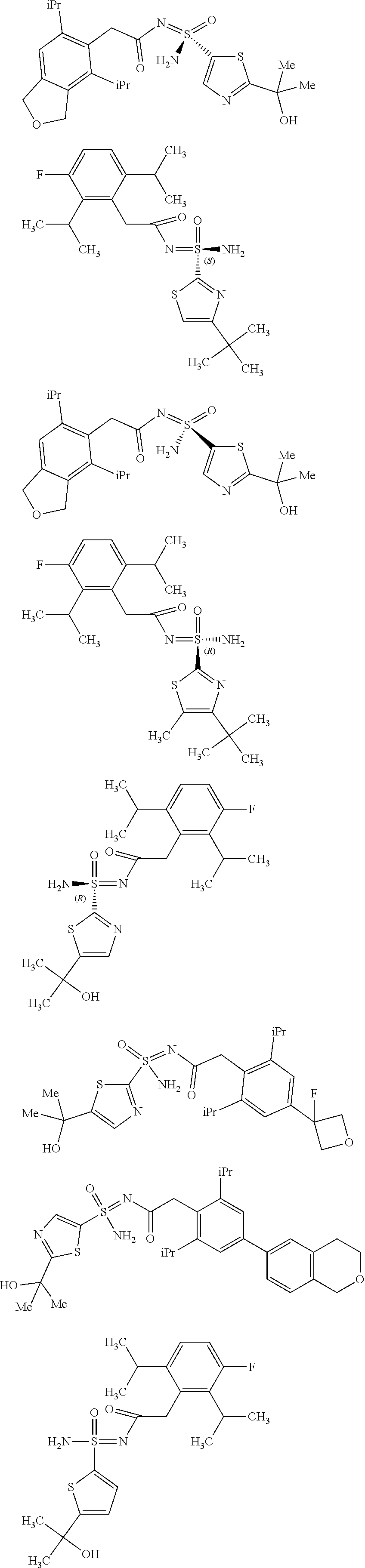

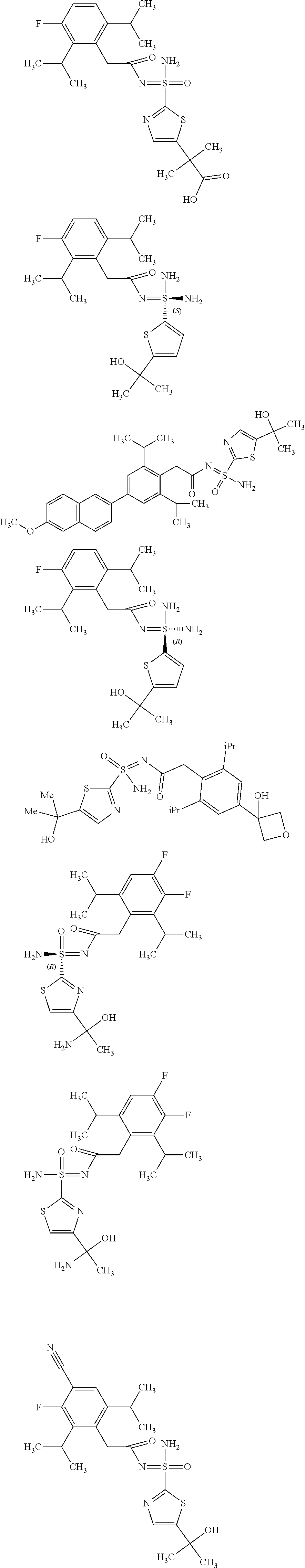

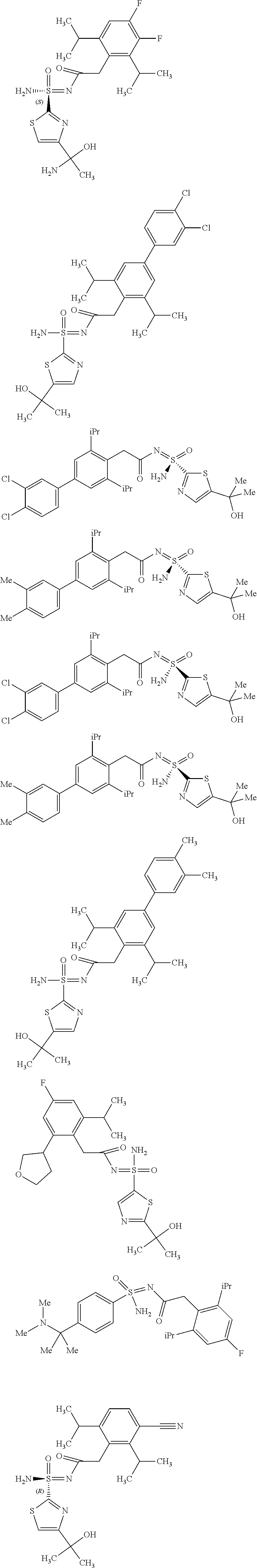

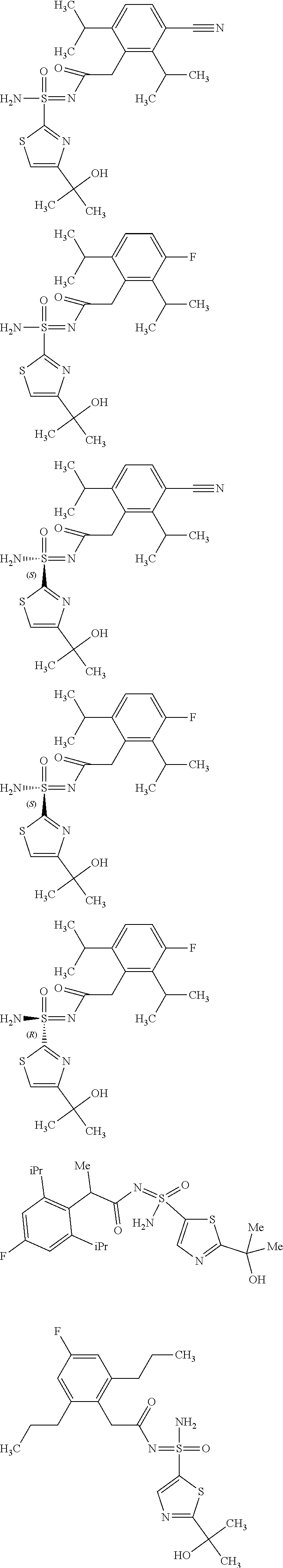

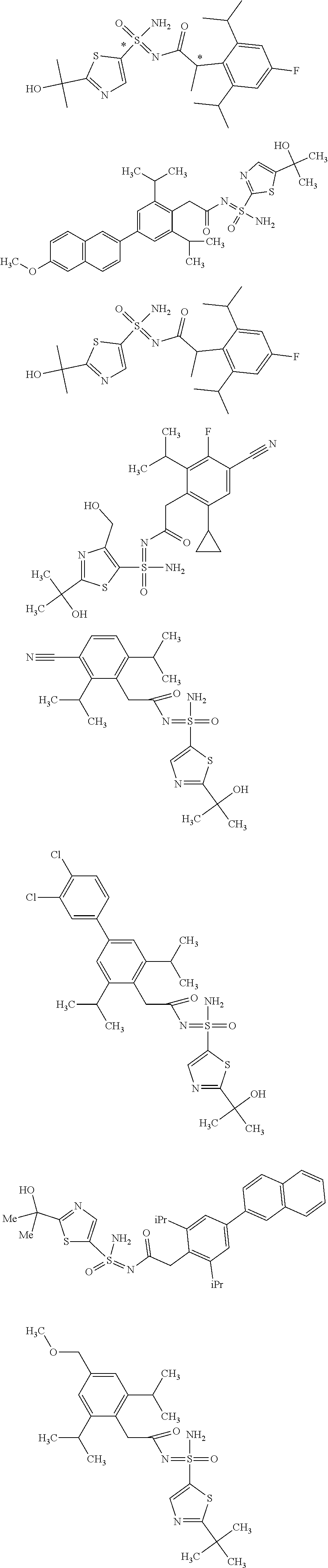

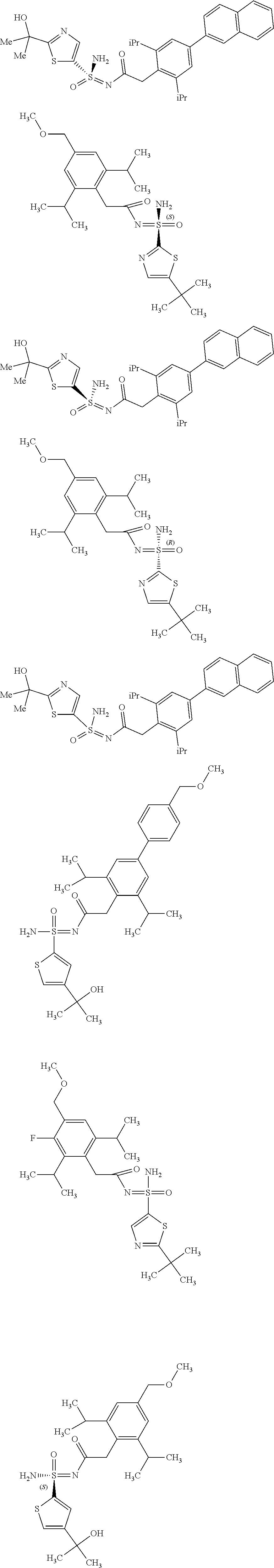

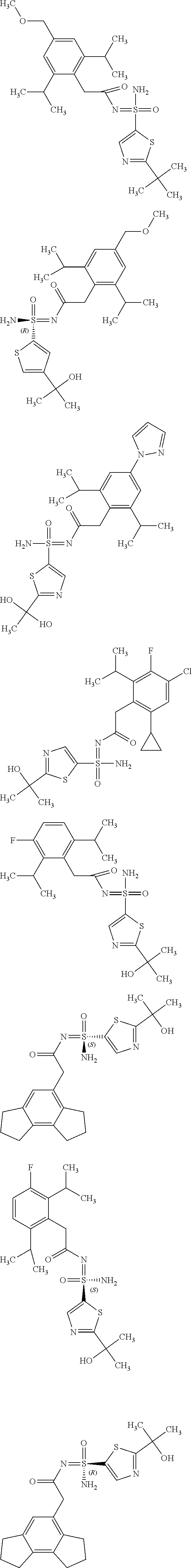

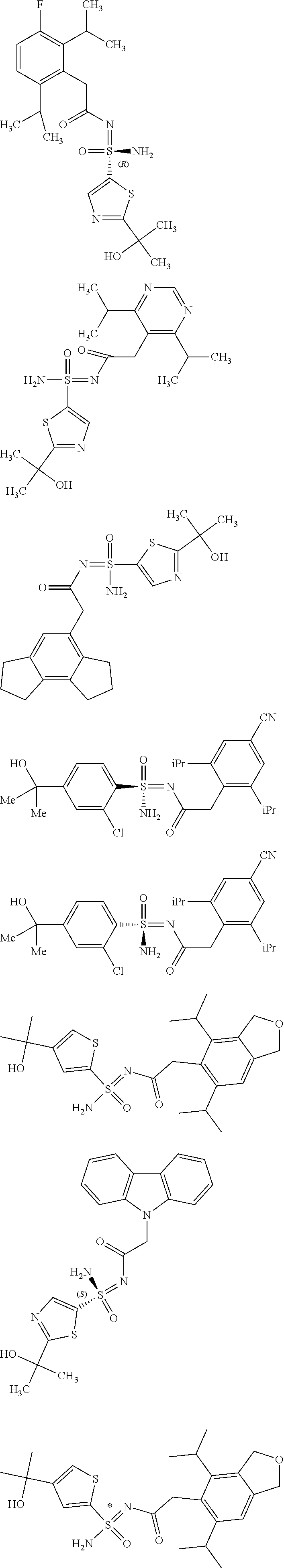

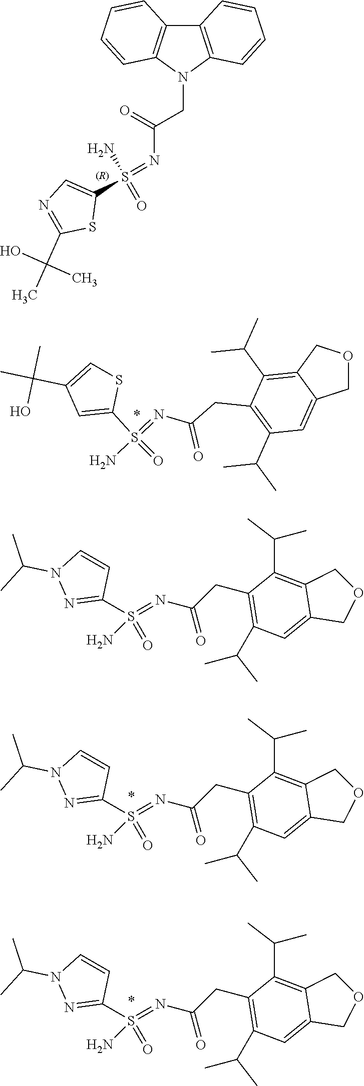

or a pharmaceutically acceptable salt thereof.

In any of the embodiments described herein, the compound of Formula AA is not one of the following:

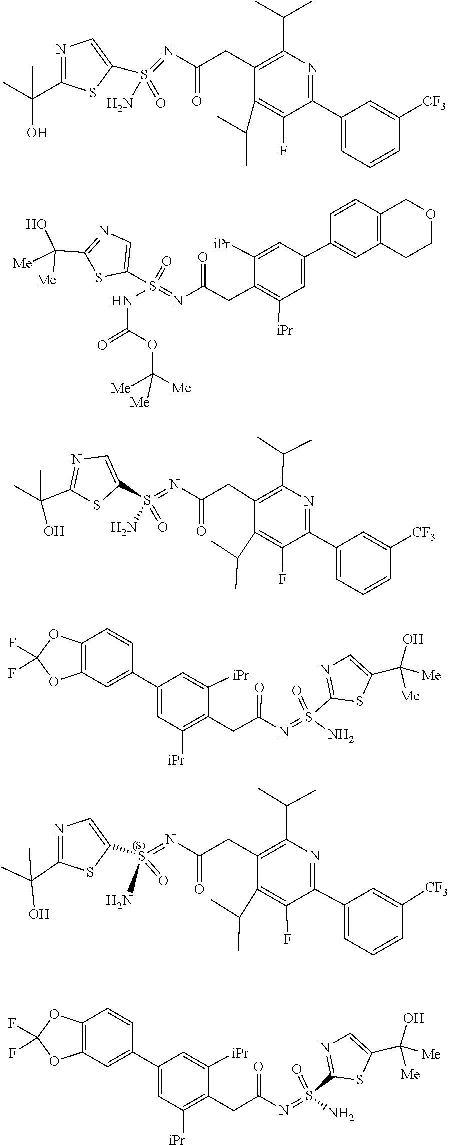

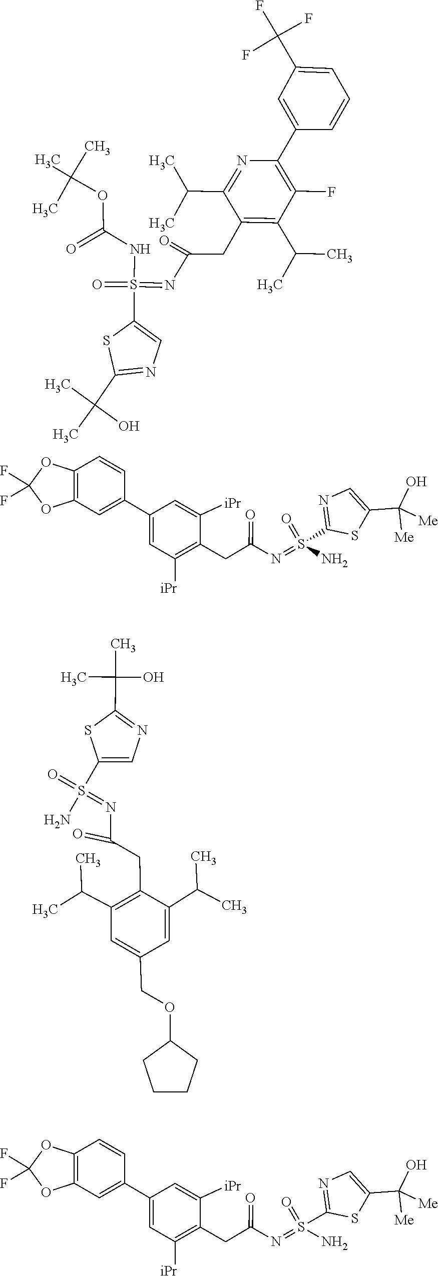

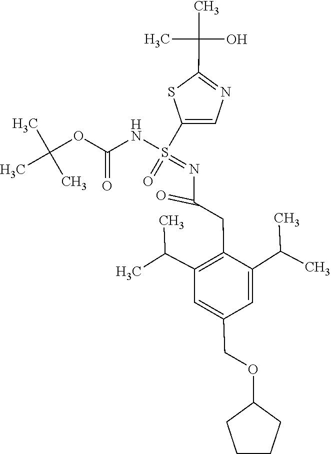 [SMILES: CC(C)c1cc(COC2CCCC2)cc(C(C)C)c1CC(=O)N=S(=O)(NC(=O)OC(C)(C)C)c1cnc(C(C)(C)O)s1]

or a pharmaceutically acceptable salt thereof.

In one embodiment, provided herein is a pharmaceutical composition comprising any NLRP3 antagonist species defined here (for example, a compound or example defined herein, in particular any one of those referred to in Tables B1, B2 and B3), and an anti-TNFα agent disclosed herein. Preferably wherein the anti-TNFα agent is Infliximab, Etanercept, Certolizumab pegol, Golimumab or Adalimumab, more preferably wherein the anti-TNFα agent is Adalimumab.

In one embodiment, provided herein is a pharmaceutical combination of a compound of any NLRP3 antagonist species defined here (for example, a compound or example defined herein, in particular any one of those referred to in Tables B1, B2 and B3), and an anti-TNFα agent Preferably wherein the anti-TNFα agent is Infliximab, Etanercept, Certolizumab pegol, Golimumab or Adalimumab, more preferably wherein the anti-TNFα agent is Adalimumab.

Pharmaceutical Compositions and Administration

General

In some embodiments, a chemical entity (e.g., a compound that modulates (e.g., antagonizes) NLRP3, or a pharmaceutically acceptable salt, and/or hydrate, and/or cocrystal, and/or drug combination thereof) is administered as a pharmaceutical composition that includes the chemical entity and one or more pharmaceutically acceptable excipients, and optionally one or more additional therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, UK. 2012).

In some embodiments, an NLRP3 antagonist and/or an anti-TNFα agent disclosed herein is administered as a pharmaceutical composition that includes the NLRP3 antagonist and/or anti-TNFα agent and one or more pharmaceutically acceptable excipients, and optionally one or more additional therapeutic agents as described herein. Preferably the pharmaceutical composition that includes an NLRP3 antagonist and an anti-TNFα agent.

Preferably the above pharmaceutical composition embodiments comprise an NLRP3 antagonist disclosed herein. More preferably the above pharmaceutical composition embodiments comprise an NLRP3 antagonist and an anti-TNFα agent disclosed herein.

Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

In certain embodiments, the chemical entities described herein or a pharmaceutical composition thereof are suitable for local, topical administration to the digestive or GI tract, e.g., rectal administration. Rectal compositions include, without limitation, enemas, rectal gels, rectal foams, rectal aerosols, suppositories, jelly suppositories, and enemas (e.g., retention enemas).

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein or a pharmaceutical composition thereof are suitable for local delivery to the digestive or GI tract by way of oral administration (e.g., solid or liquid dosage forms).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

Enema Formulations

In some embodiments, enema formulations containing the chemical entities described herein are provided in “ready-to-use” form.

In some embodiments, enema formulations containing the chemical entities described herein are provided in one or more kits or packs. In certain embodiments, the kit or pack includes two or more separately contained/packaged components, e.g. two components, which when mixed together, provide the desired formulation (e.g., as a suspension). In certain of these embodiments, the two component system includes a first component and a second component, in which: (i) the first component (e.g., contained in a sachet) includes the chemical entity (as described anywhere herein) and optionally one or more pharmaceutically acceptable excipients (e.g., together formulated as a solid preparation, e.g., together formulated as a wet granulated solid preparation); and (ii) the second component (e.g., contained in a vial or bottle) includes one or more liquids and optionally one or more other pharmaceutically acceptable excipients together forming a liquid carrier. Prior to use (e.g., immediately prior to use), the contents of (i) and (ii) are combined to form the desired enema formulation, e.g., as a suspension. In other embodiments, each of component (i) and (ii) is provided in its own separate kit or pack.

In some embodiments, each of the one or more liquids is water, or a physiologically acceptable solvent, or a mixture of water and one or more physiologically acceptable solvents. Typical such solvents include, without limitation, glycerol, ethylene glycol, propylene glycol, polyethylene glycol and polypropylene glycol. In certain embodiments, each of the one or more liquids is water. In other embodiments, each of the one or more liquids is an oil, e.g. natural and/or synthetic oils that are commonly used in pharmaceutical preparations.

Further pharmaceutical excipients and carriers that may be used in the pharmaceutical products herein described are listed in various handbooks (e.g. D. E. Bugay and W. P. Findlay (Eds) Pharmaceutical excipients (Marcel Dekker, New York, 1999), E-M Hoepfner, A. Reng and P. C. Schmidt (Eds) Fiedler Encyclopedia of Excipients for Pharmaceuticals, Cosmetics and Related Areas (Edition Cantor, Munich, 2002) and H. P. Fielder (Ed) Lexikon der Hilfsstoffe für Pharmazie, Kosmetik and angrenzende Gebiete (Edition Cantor Aulendorf, 1989)).

In some embodiments, each of the one or more pharmaceutically acceptable excipients can be independently selected from thickeners, viscosity enhancing agents, bulking agents, mucoadhesive agents, penetration enhancers, buffers, preservatives, diluents, binders, lubricants, glidants, disintegrants, fillers, solubilizing agents, pH modifying agents, preservatives, stabilizing agents, anti-oxidants, wetting or emulsifying agents, suspending agents, pigments, colorants, isotonic agents, chelating agents, emulsifiers, and diagnostic agents.

In certain embodiments, each of the one or more pharmaceutically acceptable excipients can be independently selected from thickeners, viscosity enhancing agents, mucoadhesive agents, buffers, preservatives, diluents, binders, lubricants, glidants, disintegrants, and fillers.

In certain embodiments, each of the one or more pharmaceutically acceptable excipients can be independently selected from thickeners, viscosity enhancing agents, bulking agents, mucoadhesive agents, buffers, preservatives, and fillers.

In certain embodiments, each of the one or more pharmaceutically acceptable excipients can be independently selected from diluents, binders, lubricants, glidants, and disintegrants.

Examples of thickeners, viscosity enhancing agents, and mucoadhesive agents include without limitation: gums, e.g. xanthan gum, guar gum, locust bean gum, tragacanth gums, karaya gum, ghatti gum, cholla gum, psyllium seed gum and gum arabic; poly(carboxylic acid-containing) based polymers, such as poly (acrylic, maleic, itaconic, citraconic, hydroxyethyl methacrylic or methacrylic) acid which have strong hydrogen-bonding groups, or derivatives thereof such as salts and esters; cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof; clays such as manomorillonite clays, e.g. Veegun, attapulgite clay; polysaccharides such as dextran, pectin, amylopectin, agar, mannan or polygalactonic acid or starches such as hydroxypropyl starch or carboxymethyl starch; polypeptides such as casein, gluten, gelatin, fibrin glue; chitosan, e.g. lactate or glutamate or carboxymethyl chitin; glycosaminoglycans such as hyaluronic acid; metals or water soluble salts of alginic acid such as sodium alginate or magnesium alginate; schleroglucan; adhesives containing bismuth oxide or aluminium oxide; atherocollagen; polyvinyl polymers such as carboxyvinyl polymers; polyvinylpyrrolidone (povidone); polyvinyl alcohol; polyvinyl acetates, polyvinylmethyl ethers, polyvinyl chlorides, polyvinylidenes, and/or the like; polycarboxylated vinyl polymers such as polyacrylic acid as mentioned above; polysiloxanes; polyethers; polyethylene oxides and glycols; polyalkoxys and polyacrylamides and derivatives and salts thereof. Preferred examples can include cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof (e.g., methyl cellulose); and polyvinyl polymers such as polyvinylpyrrolidone (povidone).

Examples of preservatives include without limitation: benzalkonium chloride, benzoxonium chloride, benzethonium chloride, cetrimide, sepazonium chloride, cetylpyridinium chloride, domiphen bromide (Bradosol®), thiomersal, phenylmercuric nitrate, phenylmercuric acetate, phenylmercuric borate, methylparaben, propylparaben, chlorobutanol, benzyl alcohol, phenyl ethyl alcohol, chlorohexidine, polyhexamethylene biguanide, sodium perborate, imidazolidinyl urea, sorbic acid, Purite®), Polyquart®), and sodium perborate tetrahydrate and the like.

In certain embodiments, the preservative is a paraben, or a pharmaceutically acceptable salt thereof. In some embodiments, the paraben is an alkyl substituted 4-hydroxybenzoate, or a pharmaceutically acceptable salt or ester thereof. In certain embodiments, the alkyl is a C1-C4 alkyl. In certain embodiments, the preservative is methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof.

Examples of buffers include without limitation: phosphate buffer system (sodium dihydrogen phosphate dehydrate, disodium phosphate dodecahydrate, bibasic sodium phosphate, anhydrous monobasic sodium phosphate), bicarbonate buffer system, and bisulfate buffer system.

Examples of disintegrants include, without limitation: carmellose calcium, low substituted hydroxypropyl cellulose (L-HPC), carmellose, croscarmellose sodium, partially pregelatinized starch, dry starch, carboxymethyl starch sodium, crospovidone, polysorbate 80 (polyoxyethylenesorbitan oleate), starch, sodium starch glycolate, hydroxypropyl cellulose pregelatinized starch, clays, cellulose, alginine, gums or cross linked polymers, such as crosslinked PVP (Polyplasdone XL from GAF Chemical Corp). In certain embodiments, the disintegrant is crospovidone.

Examples of glidants and lubricants (aggregation inhibitors) include without limitation: talc, magnesium stearate, calcium stearate, colloidal silica, stearic acid, aqueous silicon dioxide, synthetic magnesium silicate, fine granulated silicon oxide, starch, sodium laurylsulfate, boric acid, magnesium oxide, waxes, hydrogenated oil, polyethylene glycol, sodium benzoate, stearic acid glycerol behenate, polyethylene glycol, and mineral oil. In certain embodiments, the glidant/lubricant is magnesium stearate, talc, and/or colloidal silica; e.g., magnesium stearate and/or talc.

Examples of diluents, also referred to as “fillers” or “bulking agents” include without limitation: dicalcium phosphate dihydrate, calcium sulfate, lactose (e.g., lactose monohydrate), sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. In certain embodiments, the diluent is lactose (e.g., lactose monohydrate).

Examples of binders include without limitation: starch, pregelatinized starch, gelatin, sugars (including sucrose, glucose, dextrose, lactose and sorbitol), polyethylene glycol, waxes, natural and synthetic gums such as acacia tragacanth, sodium alginate cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone (povidone). In certain embodiments, the binder is polyvinylpyrrolidone (povidone).

In some embodiments, enema formulations containing the chemical entities described herein include water and one or more (e.g., all) of the following excipients:
One or more (e.g., one, two, or three) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);
One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof;
One or more (e.g., one or two; e.g., two) buffers, such as phosphate buffer system (e.g., sodium dihydrogen phosphate dehydrate, disodium phosphate dodecahydrate);
One or more (e.g., one or two, e.g., two) glidants and/or lubricants, such as magnesium stearate and/or talc;
One or more (e.g., one or two; e.g., one) disintegrants, such as crospovidone; and
One or more (e.g., one or two; e.g., one) diluents, such as lactose (e.g., lactose monohydrate).

In certain of these embodiments, the chemical entity is a compound of Formula AA, or a pharmaceutically acceptable salt and/or hydrate and/or cocrystal thereof.

In certain embodiments, enema formulations containing the chemical entities described herein include water, methyl cellulose, povidone, methylparaben, propylparaben, sodium dihydrogen phosphate dehydrate, disodium phosphate dodecahydrate, crospovidone, lactose monohydrate, magnesium stearate, and talc. In certain of these embodiments, the chemical entity is a compound of Formula AA, or a pharmaceutically acceptable salt and/or hydrate and/or cocrystal thereof.

In certain embodiments, enema formulations containing the chemical entities described herein are provided in one or more kits or packs. In certain embodiments, the kit or pack includes two separately contained/packaged components, which when mixed together, provide the desired formulation (e.g., as a suspension). In certain of these embodiments, the two component system includes a first component and a second component, in which: (i) the first component (e.g., contained in a sachet) includes the chemical entity (as described anywhere herein) and one or more pharmaceutically acceptable excipients (e.g., together formulated as a solid preparation, e.g., together formulated as a wet granulated solid preparation); and (ii) the second component (e.g., contained in a vial or bottle) includes one or more liquids and one or more one or more other pharmaceutically acceptable excipients together forming a liquid carrier. In other embodiments, each of component (i) and (ii) is provided in its own separate kit or pack.

In certain of these embodiments, component (i) includes the chemical entity (e.g., a compound of Formula AA, or a pharmaceutically acceptable salt and/or hydrate and/or cocrystal thereof, e.g., a compound of Formula AA) and one or more (e.g., all) of the following excipients:
(a) One or more (e.g., one) binders (e.g., a polyvinyl polymer, such as polyvinylpyrrolidone (povidone);
(b) One or more (e.g., one or two, e.g., two) glidants and/or lubricants, such as magnesium stearate and/or talc;
(c) One or more (e.g., one or two; e.g., one) disintegrants, such as crospovidone; and
(d) One or more (e.g., one or two; e.g., one) diluents, such as lactose (e.g., lactose monohydrate).

In certain embodiments, component (i) includes from about 40 weight percent to about 80 weight percent (e.g., from about 50 weight percent to about 70 weight percent, from about 55 weight percent to about 70 weight percent; from about 60 weight percent to about 65 weight percent; e.g., about 62.1 weight percent) of the chemical entity (e.g., a compound of Formula AA, or a pharmaceutically acceptable salt and/or hydrate and/or cocrystal thereof).

In certain embodiments, component (i) includes from about 0.5 weight percent to about 5 weight percent (e.g., from about 1.5 weight percent to about 4.5 weight percent, from about 2 weight percent to about 3.5 weight percent; e.g., about 2.76 weight percent) of the binder (e.g., povidone).

In certain embodiments, component (i) includes from about 0.5 weight percent to about 5 weight percent (e.g., from about 0.5 weight percent to about 3 weight percent, from about 1 weight percent to about 3 weight percent; about 2 weight percent e.g., about 1.9 weight percent) of the disintegrant (e.g., crospovidone).

In certain embodiments, component (i) includes from about 10 weight percent to about 50 weight percent (e.g., from about 20 weight percent to about 40 weight percent, from about 25 weight percent to about 35 weight percent; e.g., about 31.03 weight percent) of the diluent (e.g., lactose, e.g., lactose monohydrate).

In certain embodiments, component (i) includes from about 0.05 weight percent to about 5 weight percent (e.g., from about 0.05 weight percent to about 3 weight percent) of the glidants and/or lubricants.

In certain embodiments (e.g., when component (i) includes one or more lubricants, such as magnesium stearate), component (i) includes from about 0.05 weight percent to about 1 weight percent (e.g., from about 0.05 weight percent to about 1 weight percent; from about 0.1 weight percent to about 1 weight percent; from about 0.1 weight percent to about 0.5 weight percent; e.g., about 0.27 weight percent) of the lubricant (e.g., magnesium stearate).

In certain embodiments (when component (i) includes one or more lubricants, such as talc), component (i) includes from about 0.5 weight percent to about 5 weight percent (e.g., from about 0.5 weight percent to about 3 weight percent, from about 1 weight percent to about 3 weight percent; from about 1.5 weight percent to about 2.5 weight percent; from about 1.8 weight percent to about 2.2 weight percent; about 1.93 weight percent) of the lubricant (e.g., talc).

In certain of these embodiments, each of (a), (b), (c), and (d) above is present.

In certain embodiments, component (i) includes the ingredients and amounts as shown in Table A.

TABLE A

| Ingredient | Weight Percent |
|---|---|
| A compound of Formula | 40 weight percent to about 80 weight |
| AA | percent (e.g., from about 50 weight percent |
| | to about 70 weight percent, from about 55 |
| | weight percent to about 70 weight percent; |
| | from about 60 weight percent to about 65 |
| | weight percent; e.g., about 62.1 weight |
| | percent) |
| Crospovidone (Kollidon | 0.5 weight percent to about 5 weight |
| CL) | percent (e.g., from about 0.5 weight |
| | percent to about 3 weight percent, from |
| | about 1 weight percent to about 3 weight |
| | percent; about 1.93 weight percent |
| lactose monohydrate | about 10 weight percent to about 50 weight |
| (Pharmatose 200M) | percent (e.g., from about 20 weight percent |
| | to about 40 weight percent, from about 25 |
| | weight percent to about 35 weight percent; |
| | e.g., about 31.03 weight percent |
| Povidone (Kollidon K30) | about 0.5 weight percent to about 5 weight |
| | percent (e.g., from about 1.5 weight |
| | percent to about 4.5 weight percent, from |
| | about 2 weight percent to about 3.5 weight |
| | percent; e.g., about 2.76 weight percent |
| talc | 0.5 weight percent to about 5 weight |
| | percent (e.g., from about 0.5 weight |
| | percent to about 3 weight percent, from |
| | about 1 weight percent to about 3 weight |
| | percent; from about 1.5 weight percent to |
| | about 2.5 weight percent; from about 1.8 |
| | weight percent to about 2.2 weight |
| | percent; e.g., about 1.93 weight percent |
| Magnesium stearate | about 0.05 weight percent to about 1 |
| | weight percent (e.g., from about 0.05 |
| | weight percent to about 1 weight percent; |
| | from about 0.1 weight percent to about 1 |
| | weight percent; from about 0.1 weight |
| | percent to about 0.5 weight percent; e.g., |
| | about 0.27 weight percent |

In certain embodiments, component (i) includes the ingredients and amounts as shown in Table B.

TABLE B

| Ingredient | Weight Percent |
|---|---|
| A compound of Formula AA | About 62.1 weight percent) |
| Crospovidone (Kollidon CL) | About 1.93 weight percent |
| lactose monohydrate (Pharmatose 200M) | About 31.03 weight percent |
| Povidone (Kollidon K30) | About 2.76 weight percent |
| talc | About 1.93 weight percent |
| Magnesium stearate | About 0.27 weight percent |

In certain embodiments, component (i) is formulated as a wet granulated solid preparation. In certain of these embodiments an internal phase of ingredients (the chemical entity, disintegrant, and diluent) are combined and mixed in a high-shear granulator. A binder (e.g., povidone) is dissolved in water to form a granulating solution. This solution is added to the Inner Phase mixture resulting in the development of granules. While not wishing to be bound by theory, granule development is believed to be facilitated by the interaction of the polymeric binder with the materials of the internal phase. Once the granulation is formed and dried, an external phase (e.g., one or more lubricants—not an intrinsic component of the dried granulation), is added to the dry granulation. It is believed that lubrication of the granulation is important to the flowability of the granulation, in particular for packaging.

In certain of the foregoing embodiments, component (ii) includes water and one or more (e.g., all) of the following excipients:
(a′) One or more (e.g., one, two; e.g., two) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);
(b′) One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof, and
(c′) One or more (e.g., one or two; e.g., two) buffers, such as phosphate buffer system (e.g., sodium dihydrogen phosphate dihydrate, disodium phosphate dodecahydrate);

In certain of the foregoing embodiments, component (ii) includes water and one or more (e.g., all) of the following excipients:
(a″) a first thickener, viscosity enhancing agent, binder, and/or mucoadhesive agent (e.g., a cellulose or cellulose ester or ether or derivative or salt thereof (e.g., methyl cellulose));
(a′″) a second thickener, viscosity enhancing agent, binder, and/or mucoadhesive agent (e.g., a polyvinyl polymer, such as polyvinylpyrrolidone (povidone));
(b″) a first preservative, such as a paraben, e.g., propyl 4-hydroxybenzoate (propylparaben), or a pharmaceutically acceptable salt or ester thereof;
(b″) a second preservative, such as a paraben, e.g., methyl 4-hydroxybenzoate (methylparaben), or a pharmaceutically acceptable salt or ester thereof,
(c″) a first buffer, such as phosphate buffer system (e.g., disodium phosphate dodecahydrate);
(c′″) a second buffer, such as phosphate buffer system (e.g., sodium dihydrogen phosphate dehydrate),

In certain embodiments, component (ii) includes from about 0.05 weight percent to about 5 weight percent (e.g., from about 0.05 weight percent to about 3 weight percent, from about 0.1 weight percent to about 3 weight percent; e.g., about 1.4 weight percent) of (a″).

In certain embodiments, component (ii) includes from about 0.05 weight percent to about 5 weight percent (e.g., from about 0.05 weight percent to about 3 weight percent, from about 0.1 weight percent to about 2 weight percent; e.g., about 1.0 weight percent) of (a′″).

In certain embodiments, component (ii) includes from about 0.005 weight percent to about 0.1 weight percent (e.g., from about 0.005 weight percent to about 0.05 weight percent; e.g., about 0.02 weight percent) of (b″).

In certain embodiments, component (ii) includes from about 0.05 weight percent to about 1 weight percent (e.g., from about 0.05 weight percent to about 0.5 weight percent; e.g., about 0.20 weight percent) of (b′″).

In certain embodiments, component (ii) includes from about 0.05 weight percent to about 1 weight percent (e.g., from about 0.05 weight percent to about 0.5 weight percent; e.g., about 0.15 weight percent) of (c″).

In certain embodiments, component (ii) includes from about 0.005 weight percent to about 0.5 weight percent (e.g., from about 0.005 weight percent to about 0.3 weight percent; e.g., about 0.15 weight percent) of (c′″).

In certain of these embodiments, each of (a″)-(c′″) is present.

In certain embodiments, component (ii) includes water (up to 100%) and the ingredients and amounts as shown in Table C.

TABLE C

| Ingredient | Weight Percent |
|---|---|
| methyl cellulose | 0.05 weight percent to about 5 weight |
| (Methocel A15C premium) | percent (e.g., from about 0.05 weight |
| | percent to about 3 weight percent, from |
| | about 0.1 weight percent to about 3 weight |
| | percent; e.g., about 1.4 weight percent |
| Povidone (Kollidon K30) | 0.05 weight percent to about 5 weight |
| | percent (e.g., from about 0.05 weight |
| | percent to about 3 weight percent, from |
| | about 0.1 weight percent to about 2 weight |
| | percent; e.g., about 1.0 weight percent |
| propyl 4-hydroxybenzoate | about 0.005 weight percent to about 0.1 |
| | weight percent (e.g., from about 0.005 |
| | weight percent to about 0.05 weight |
| | percent; e.g., about 0.02 weight percent) |
| methyl 4-hydroxybenzoate | about 0.05 weight percent to about 1 |
| | weight percent (e.g., from about 0.05 |
| | weight percent to about 0.5 weight percent; |
| | e.g., about 0.20 weight percent) |
| disodium phosphate | about 0.05 weight percent to about 1 |
| dodecahydrate | weight percent (e.g., from about 0.05 |
| | weight percent to about 0.5 weight percent; |
| | e.g., about 0.15 weight percent) |
| sodium dihydrogen | about 0.005 weight percent to about 0.5 |
| phospahate dihydrate | weight percent (e.g., from about 0.005 |
| | weight percent to about 0.3 weight percent; |
| | e.g., about 0.15 weight percent) |

In certain embodiments, component (ii) includes water (up to 1000%) and the ingredients and amounts as shown in Table D.

TABLE D

| Ingredient | Weight Percent |
|---|---|
| methyl cellulose (Methocel A15C premium) | about 1.4 weight percent |
| Povidone (Kollidon K30) | about 1.0 weight percent |
| propyl 4-hydroxybenzoate | about 0.02 weight percent |
| methyl 4-hydroxybenzoate | about 0.20 weight percent |
| disodium phosphate dodecahydrate | about 0.15 weight percent |
| sodium dihydrogen phospahate dihydrate | about 0.15 weight percent |

Ready-to-use” enemas are generally be provided in a “single-use” sealed disposable container of plastic or glass. Those formed of a polymeric material preferably have sufficient flexibility for ease of use by an unassisted patient. Typical plastic containers can be made of polyethylene. These containers may comprise a tip for direct introduction into the rectum. Such containers may also comprise a tube between the container and the tip. The tip is preferably provided with a protective shield which is removed before use. Optionally the tip has a lubricant to improve patient compliance.

In some embodiments, the enema formulation (e.g., suspension) is poured into a bottle for delivery after it has been prepared in a separate container. In certain embodiments, the bottle is a plastic bottle (e.g., flexible to allow for delivery by squeezing the bottle), which can be a polyethylene bottle (e.g., white in color). In some embodiments, the bottle is a single chamber bottle, which contains the suspension or solution. In other embodiments, the bottle is a multichamber bottle, where each chamber contains a separate mixture or solution. In still other embodiments, the bottle can further include a tip or rectal cannula for direct introduction into the rectum.

Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/Kg to about 500 mg/Kg (e.g., from about 0.001 mg/Kg to about 200 mg/Kg; from about 0.01 mg/Kg to about 200 mg/Kg; from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg).

In some embodiments, enema formulations include from about 0.5 mg to about 2500 mg (e.g., from about 0.5 mg to about 2000 mg, from about 0.5 mg to about 1000 mg, from about 0.5 mg to about 750 mg, from about 0.5 mg to about 600 mg, from about 0.5 mg to about 500 mg, from about 0.5 mg to about 400 mg, from about 0.5 mg to about 300 mg, from about 0.5 mg to about 200 mg; e.g., from about 5 mg to about 2500 mg, from about 5 mg to about 2000 mg, from about 5 mg to about 1000 mg; from about 5 mg to about 750 mg; from about 5 mg to about 600 mg; from about 5 mg to about 500 mg; from about 5 mg to about 400 mg; from about 5 mg to about 300 mg; from about 5 mg to about 200 mg; e.g., from about 50 mg to about 2000 mg, from about 50 mg to about 1000 mg, from about 50 mg to about 750 mg, from about 50 mg to about 600 mg, from about 50 mg to about 500 mg, from about 50 mg to about 400 mg, from about 50 mg to about 300 mg, from about 50 mg to about 200 mg; e.g., from about 100 mg to about 2500 mg, from about 100 mg to about 2000 mg, from about 100 mg to about 1000 mg, from about 100 mg to about 750 mg, from about 100 mg to about 700 mg, from about 100 mg to about 600 mg, from about 100 mg to about 500 mg, from about 100 mg to about 400 mg, from about 100 mg to about 300 mg, from about 100 mg to about 200 mg; e.g., from about 150 mg to about 2500 mg, from about 150 mg to about 2000 mg, from about 150 mg to about 1000 mg, from about 150 mg to about 750 mg, from about 150 mg to about 700 mg, from about 150 mg to about 600 mg, from about 150 mg to about 500 mg, from about 150 mg to about 400 mg, from about 150 mg to about 300 mg, from about 150 mg to about 200 mg; e.g., from about 150 mg to about 500 mg; e.g., from about 300 mg to about 2500 mg, from about 300 mg to about 2000 mg, from about 300 mg to about 1000 mg, from about 300 mg to about 750 mg, from about 300 mg to about 700 mg, from about 300 mg to about 600 mg; e.g., from about 400 mg to about 2500 mg, from about 400 mg to about 2000 mg, from about 400 mg to about 1000 mg, from about 400 mg to about 750 mg, from about 400 mg to about 700 mg, from about 400 mg to about 600 from about 400 mg to about 500 mg; e.g., 150 mg or 450 mg) of the chemical entity in from about 1 mL to about 3000 mL (e.g., from about 1 mL to about 2000 mL, from about 1 mL to about 1000 mL, from about 1 mL to about 500 mL, from about 1 mL to about 250 mL, from about 1 mL to about 100 mL, from about 10 mL to about 1000 mL, from about 10 mL to about 500 mL, from about 10 mL to about 250 mL, from about 10 mL to about 100 mL, from about 30 mL to about 90 mL, from about 40 mL to about 80 mL; from about 50 mL to about 70 mL; e.g., about 1 mL, about 5 mL, about 10 mL, about 15 mL, about 20 mL, about 25 mL, about 30 mL, about 35 mL, about 40 mL, about 45 mL, about 50 mL, about 55 mL, about 60 mL, about 65 mL, about 70 mL, about 75 mL, about 100 mL, about 250 mL, or about 500 mL, or about 1000 mL, or about 2000 mL, or about 3000 mL; e.g., 60 mL) of liquid carrier.

In certain embodiments, enema formulations include from about 50 mg to about 250 mg (e.g., from about 100 mg to about 200; e.g., about 150 mg) of the chemical entity in from about 10 mL to about 100 mL (e.g., from about 20 mL to about 100 mL, from about 30 mL to about 90 mL, from about 40 mL to about 80 mL; from about 50 mL to about 70 mL) of liquid carrier. In certain embodiments, enema formulations include about 150 mg of the chemical entity in about 60 mL of the liquid carrier. In certain of these embodiments, the chemical entity is a compound of Formula AA, or a pharmaceutically acceptable salt and/or hydrate and/or cocrystal thereof. For example, enema formulations can include about 150 mg of a compound of Formula AA in about 60 mL of the liquid carrier.

In certain embodiments, enema formulations include from about 350 mg to about 550 mg (e.g., from about 400 mg to about 500; e.g., about 450 mg) of the chemical entity in from about 10 mL to about 100 mL (e.g., from about 20 mL to about 100 mL, from about 30 mL to about 90 mL, from about 40 mL to about 80 mL; from about 50 mL to about 70 mL) of liquid carrier. In certain embodiments, enema formulations include about 450 mg of the chemical entity in about 60 mL of the liquid carrier. In certain of these embodiments, the chemical entity is a compound of Formula AA, or a pharmaceutically acceptable salt and/or hydrate and/or cocrystal thereof. For example, enema formulations can include about 450 mg of a compound of Formula AA in about 60 mL of the liquid carrier.

In some embodiments, enema formulations include from about from about 0.01 mg/mL to about 50 mg/mL (e.g., from about 0.01 mg/mL to about 25 mg/mL; from about 0.01 mg/mL to about 10 mg/mL; from about 0.01 mg/mL to about 5 mg/mL; from about 0.1 mg/mL to about 50 mg/mL; from about 0.01 mg/mL to about 25 mg/mL; from about 0.1 mg/mL to about 10 mg/mL; from about 0.1 mg/mL to about 5 mg/mL; from about 1 mg/mL to about 10 mg/mL; from about 1 mg/mL to about 5 mg/mL; from about 5 mg/mL to about 10 mg/mL; e.g., about 2.5 mg/mL or about 7.5 mg/mL) of the chemical entity in liquid carrier. In certain of these embodiments, the chemical entity is a compound of Formula AA, or a pharmaceutically acceptable salt and/or hydrate and/or cocrystal thereof. For example, enema formulations can include about 2.5 mg/mL or about 7.5 mg/mL of a compound of Formula AA in liquid carrier.

Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

Methods of Treatment

In some embodiments, methods for treating a subject having condition, disease or disorder in which a decrease or increase in NLRP3 activity (e.g., an increase, e.g., NLRP3 signaling) contributes to the pathology and/or symptoms and/or progression of the condition, disease or disorder are provided, comprising administering to a subject an effective amount of a chemical entity described herein (e.g., a compound described generically or specifically herein or a pharmaceutically acceptable salt thereof or compositions containing the same).

Indications

In some embodiments, the condition, disease or disorder is selected from: inappropriate host responses to infectious diseases where active infection exists at any body site, such as septic shock, disseminated intravascular coagulation, and/or adult respiratory distress syndrome; acute or chronic inflammation due to antigen, antibody and/or complement deposition; inflammatory conditions including arthritis, cholangitis, colitis, encephalitis, endocarditis, glomerulonephritis, hepatitis, myocarditis, pancreatitis, pericarditis, reperfusion injury and vasculitis, immune-based diseases such as acute and delayed hypersensitivity, graft rejection, and graft-versus-host disease; auto-immune diseases including Type 1 diabetes mellitus and multiple sclerosis. For example, the condition, disease or disorder may be an inflammatory disorder such as rheumatoid arthritis, osteoarthritis, septic shock, COPD and periodontal disease.

In some embodiments, the condition, disease or disorder is an autoimmune diseases. Non-limiting examples include rheumatoid arthritis, systemic lupus erythematosus, multiple sclerosis, inflammatory bowel diseases (IBDs) comprising Crohn disease (CD) and ulcerative colitis (UC), which are chronic inflammatory conditions with polygenic susceptibility. In certain embodiments, the condition is an inflammatory bowel disease. In certain embodiments, the condition is Crohn's disease, autoimmune colitis, iatrogenic autoimmune colitis, ulcerative colitis, colitis induced by one or more chemotherapeutic agents, colitis induced by treatment with adoptive cell therapy, colitis associated by one or more alloimmune diseases (such as graft-vs-host disease, e.g., acute graft vs. host disease and chronic graft vs. host disease), radiation enteritis, collagenous colitis, lymphocytic colitis, microscopic colitis, and radiation enteritis. In certain of these embodiments, the condition is alloimmune disease (such as graft-vs-host disease, e.g., acute graft vs. host disease and chronic graft vs. host disease), celiac disease, irritable bowel syndrome, rheumatoid arthritis, lupus, scleroderma, psoriasis, cutaneous T-cell lymphoma, uveitis, and mucositis (e.g., oral mucositis, esophageal mucositis or intestinal mucositis).

In some embodiments, the condition, disease or disorder is selected from major adverse cardiovascular events such as cardiovascular death, non-fatal myocardial infarction and non-fatal stroke in patients with a prior hear attack and inflammatory atherosclerosis (see for example, NCT01327846).

In some embodiments, the condition, disease or disorder is selected from metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as Osteoarthritis, osteoporosis and osteopetrosis disorders eye disease, such as glaucoma and macular degeneration, diseased caused by viral infection such as HIV and AIDS, autoimmune disease such as Rheumatoid Arthritis, Systemic Lupus Erythematosus, Autoimmune Thyroiditis, Addison's disease, pernicious anemia, cancer and aging.

In some embodiments, the condition, disease or disorder is a cardiovascular indication. In some embodiments, the condition, disease or disorder is myocardial infraction. In some embodiments, the condition, disease or disorder is stroke.

In some embodiments, the condition, disease or disorder is obesity.

In some embodiments, the condition, disease or disorder is Type 2 Diabetes.

In some embodiments, the condition, disease or disorder is NASH.

In some embodiments, the condition, disease or disorder is Alzheimer's disease.

In some embodiments, the condition, disease or disorder is gout.

In some embodiments, the condition, disease or disorder is SLE.

In some embodiments, the condition, disease or disorder is rheumatoid arthritis.

In some embodiments, the condition, disease or disorder is IBD.

In some embodiments, the condition, disease or disorder is multiple sclerosis.

In some embodiments, the condition, disease or disorder is COPD.

In some embodiments, the condition, disease or disorder is asthma.

In some embodiments, the condition, disease or disorder is scleroderma.

In some embodiments, the condition, disease or disorder is pulmonary fibrosis.

In some embodiments, the condition, disease or disorder is age related macular degeneration (AMD).

In some embodiments, the condition, disease or disorder is cystic fibrosis.

In some embodiments, the condition, disease or disorder is Muckle Wells syndrome.

In some embodiments, the condition, disease or disorder is familial cold autoinflammatory syndrome (FCAS).

In some embodiments, the condition, disease or disorder is chronic neurologic cutaneous and articular syndrome.

In some embodiments, the condition, disease or disorder is selected from: myelodysplastic syndromes (MDS); non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's cell histiocytosis (LCH); multiple myeloma; promyelocytic leukemia; acute myeloid leukemia (AML) chronic myeloid leukemia (CML); gastric cancer; and lung cancer metastasis.

In some embodiments, the condition, disease or disorder is selected from: myelodysplastic syndromes (MDS); non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's cell histiocytosis (LCH); multiple myeloma; promyelocytic leukemia; gastric cancer; and lung cancer metastasis.

In some embodiments, the indication is MDS.

In some embodiments, the indication is non-small lung cancer in patients carrying mutation or overexpression of NLRP3.

In some embodiments, the indication is ALL in patients resistant to glucocorticoids treatment.

In some embodiments, the indication is LCH.

In some embodiments, the indication is multiple myeloma.

In some embodiments, the indication is promyelocytic leukemia.

In some embodiments, the indication is gastric cancer.

In some embodiments, the indication is lung cancer metastasis.

Combination Therapy

This disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In certain embodiments, the second therapeutic agent or regimen is administered to the subject prior to contacting with or administering the chemical entity (e.g., about one hour prior, or about 6 hours prior, or about 12 hours prior, or about 24 hours prior, or about 48 hours prior, or about 1 week prior, or about 1 month prior).

In other embodiments, the second therapeutic agent or regimen is administered to the subject at about the same time as contacting with or administering the chemical entity. By way of example, the second therapeutic agent or regimen and the chemical entity are provided to the subject simultaneously in the same dosage form. As another example, the second therapeutic agent or regimen and the chemical entity are provided to the subject concurrently in separate dosage forms.

In still other embodiments, the second therapeutic agent or regimen is administered to the subject after contacting with or administering the chemical entity (e.g., about one hour after, or about 6 hours after, or about 12 hours after, or about 24 hours after, or about 48 hours after, or about 1 week after, or about 1 month after).

Patient Selection

In some embodiments, the methods described herein further include the step of identifying a subject (e.g., a patient) in need of treatment for an indication related to NLRP3 activity, such as an indication related to NLRP3 polymorphism.

In some embodiments, the methods described herein further include the step of identifying a subject (e.g., a patient) in need of treatment for an indication related to NLRP3 activity, such as an indication related to NLRP3 where polymorphism is a gain of function

In some embodiments, the methods described herein further include the step of identifying a subject (e.g., a patient) in need of treatment for an indication related to NLRP3 activity, such as an indication related to NLRP3 polymorphism found in CAPS syndromes.

In some embodiments, the methods described herein further include the step of identifying a subject (e.g., a patient) in need of treatment for an indication related to NLRP3 activity, such as an indication related NLRP3 polymorphism where the polymorphism is VAR_014104 (R262W)

In some embodiments, the methods described herein further include the step of identifying a subject (e.g., a patient) in need of treatment for an indication related to NLRP3 activity, such as an indication related NLRP3 polymorphism where the polymorphism is a natural variant reported in http://www.uniprot.org/uniprot/Q96P20.

In some embodiments, the methods described herein further include the step of identifying a subject (e.g., a patient) in need of treatment for an indication related to NLRP3 activity, such as an indication related to point mutation of NLRP3 signaling.

### Anti-TNFα Agents

The term “anti-TNFα agent” refers to an agent which directly or indirectly blocks, down-regulates, impairs, inhibits, impairs, or reduces TNFα activity and/or expression. In some embodiments, an anti-TNFα agent is an antibody or an antigen-binding fragment thereof, a fusion protein, a soluble TNFα receptor (a soluble tumor necrosis factor receptor superfamily member 1A (TNFR1) or a soluble tumor necrosis factor receptor superfamily 1B (TNFR2)), an inhibitory nucleic acid, or a small molecule TNFα antagonist. In some embodiments, the inhibitory nucleic acid is a ribozyme, small hairpin RNA, a small interfering RNA, an antisense nucleic acid, or an aptamer.

Exemplary anti-TNFα agents that directly block, down-regulate, impair, inhibit, or reduce TNFα activity and/or expression can, e.g., inhibit or decrease the expression level of TNFα or a receptor of TNFα (TNFR1 or TNFR2) in a cell (e.g., a cell obtained from a subject, a mammalian cell), or inhibit or reduce binding of TNFα to its receptor (TNFR1 and/or TNFR2) and/or. Non-limiting examples of anti-TNFα agents that directly block, down-regulate, impair, inhibit, or reduce TNFα activity and/or expression include an antibody or fragment thereof, a fusion protein, a soluble TNFα receptor (e.g., a soluble TNFR1 or soluble TNFR2), inhibitory nucleic acids (e.g., any of the examples of inhibitory nucleic acids described herein), and a small molecule TNFα antagonist.

Exemplary anti-TNFα agents that can indirectly block, down-regulate, impair, inhibit reduce TNFα activity and/or expression can, e.g., inhibit or decrease the level of downstream signaling of a TNFα receptor (e.g., TNFR1 or TNFR2) in a mammalian cell (e.g., decrease the level and/or activity of one or more of the following signaling proteins: AP-1, mitogen-activated protein kinase kinase kinase 5 (ASK1), inhibitor of nuclear factor kappa B (IKK), mitogen-activated protein kinase 8 (INK), mitogen-activated protein kinase (MAPK), MEKK 1/4, MEKK 4/7, MEKK 3/6, nuclear factor kappa B (NF-κB), mitogen-activated protein kinase kinase kinase 14 (NIK), receptor interacting serine/threonine kinase 1 (RIP), TNFRSF1A associated via death domain (TRADD), and TNF receptor associated factor 2 (TRAF2), in a cell), and/or decrease the level of TNFα-induced gene expression in a mammalian cell (e.g., decrease the transcription of genes regulated by, e.g., one or more transcription factors selected from the group of activating transcription factor 2 (ATF2), c-Jun, and NF-κB). A description of downstream signaling of a TNFα receptor is provided in Wajant et al., Cell Death Differentiation 10:45-65, 2003 (incorporated herein by reference). For example, such indirect anti-TNFα agents can be an inhibitory nucleic acid that targets (decreases the expression) a signaling component downstream of a TNFα-induced gene (e.g., any TNFα-induced gene known in the art), a TNFα receptor (e.g., any one or more of the signaling components downstream of a TNFα receptor described herein or known in the art), or a transcription factor selected from the group of NF-κB, c-Jun, and ATF2.

In other examples, such indirect anti-TNFα agents can be a small molecule inhibitor of a protein encoded by a TNFα-induced gene (e.g., any protein encoded by a TNFα-induced gene known in the art), a small molecule inhibitor of a signaling component downstream of a TNFα receptor (e.g., any of the signaling components downstream of a TNFα receptor described herein or known in the art), and a small molecule inhibitor of a transcription factor selected from the group of ATF2, c-Jun, and NF-κB.

In other embodiments, anti-TNFα agents that can indirectly block, down-regulate, impair, or reduce one or more components in a cell (e.g., a cell obtained from a subject, a mammalian cell) that are involved in the signaling pathway that results in TNFα mRNA transcription, TNFα mRNA stabilization, and TNFα mRNA translation (e.g., one or more components selected from the group of CD14, c-Jun, ERK1/2, IKK, IκB, interleukin 1 receptor associated kinase 1 (IRAK), INK, lipopolysaccharide binding protein (LBP), MEK1/2, MEK3/6, MEK4/7, MK2, MyD88, NF-κB, NIK, PKR, p38, AKT serine/threonine kinase 1 (rac), raf kinase (raf), ras, TRAF6, TTP). For example, such indirect anti-TNFα agents can be an inhibitory nucleic acid that targets (decreases the expression) of a component in a mammalian cell that is involved in the signaling pathway that results in TNFα mRNA transcription, TNFα mRNA stabilization, and TNFα mRNA translation (e.g., a component selected from the group of CD14, c-Jun, ERK1/2, IKK, IκB, IRAK, JNK, LBP, MEK1/2, MEK3/6, MEK4/7, MK2, MyD88, NF-κB, NIK, IRAK, lipopolysaccharide binding protein (LBP), PKR, p38, rac, raf, ras, TRAF6, TTP). In other examples, an indirect anti-TNFα agents is a small molecule inhibitor of a component in a mammalian cell that is involved in the signaling pathway that results in TNFα mRNA transcription, TNFα mRNA stabilization, and TNFα mRNA translation (e.g., a component selected from the group of CD14, c-Jun, ERK1/2, IKK, IκB, IRAK, JNK, lipopolysaccharide binding protein (LBP), MEK1/2, MEK3/6, MEK4/7, MK2, MyD88, NF-κB, NIX, IRAK, lipopolysaccharide binding protein (LBP), PKR, p38, rac, raf, ras, TRAF6, TTP).

### Antibodies

In some embodiments, the anti-TNFα agent is an antibody or an antigen-binding fragment thereof (e.g., a Fab or a scFv). In some embodiments, an antibody or antigen-binding fragment of an antibody described herein can bind specifically to TNFα. In some embodiments, an antibody or antigen-binding fragment described herein binds specifically to any one of TNFα, TNFR1, or TNFR2. In some embodiments, an antibody or antigen-binding fragment of an antibody described herein can bind specifically to a TNFα receptor (TNFR1 or TNFR2).

In some embodiments, the antibody can be a humanized antibody, a chimeric antibody, a multivalent antibody, or a fragment thereof. In some embodiments, an antibody can be a scFv-Fc, a VHH domain, a VNAR domain, a (scFv)2, a minibody, or a BiTE.

In some embodiments, an antibody can be a crossmab, a diabody, a scDiabody, a scDiabody-CH3, a Diabody-CH3, a DutaMab, a DT-IgG, a diabody-Fc, a scDiabody-HAS, a charge pair antibody, a Fab-arm exchange antibody, a SEEDbody, a Triomab, a LUZ-Y, a Fcab, a kλ-body, an orthogonal Fab, a DVD-IgG, an IgG(H)-scFv, a scFv-(H)IgG, an IgG(L)-scFv, a scFv-(L)-IgG, an IgG (L,H)-Fc, an IgG(H)-V, a V(H)—IgG, an IgG(L)-V, a V(L)-IgG, an KIH IgG-scFab, a 2scFv-IgG, an IgG-2scFv, a scFv4-Ig, a Zybody, a DVI-IgG, a nanobody, a nanobody-HSA, a DVD-Ig, a dual-affinity re-targeting antibody (DART), a triomab, a kih IgG with a common LC, an ortho-Fab IgG, a 2-in-1-IgG, IgG-ScFv, scFv2-Fc, a bi-nanobody, tanden antibody, a DART-Fe, a scFv-HAS-scFv, a DAF (two-in-one or four-in-one), a DNL-Fab3, knobs-in-holes common LC, knobs-in-holes assembly, a TandAb, a Triple Body, a miniantibody, a minibody, a TriBi minibody, a scFv-CH3 KIH, a Fab-scFv, a scFv-CH-CL-scFv, a F(ab′)2-scFV2, a scFv-KIH, a Fab-scFv-Fc, a tetravalent HCAb, a scDiabody-Fc, a tandem scFv-Fc, an intrabody, a dock and lock bispecific antibody, an ImmTAC, a HSAbody, a tandem scFv, an IgG-IgG, a Cov-X-Body, and a scFv1-PEG-scFv2.

Non-limiting examples of an antigen-binding fragment of an antibody include an Fv fragment, a Fab fragment, a F(ab′)2 fragment, and a Fab′ fragment. Additional examples of an antigen-binding fragment of an antibody is an antigen-binding fragment of an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); or an antigen-binding fragment of an IgM (e.g., an antigen-binding fragment of a human or humanized IgM).

Non-limiting examples of anti-TNFα agents that are antibodies that specifically bind to TNFα are described in Ben-Horin et al., Autoimmunity Rev. 13(1):24-30, 2014; Bongartz et al., JAMA 295(19):2275-2285, 2006; Butler et al., Eur. Cytokine Network 6(4):225-230, 1994; Cohen et al., Canadian J. Gastroenterol. Hepatol. 15(6):376-384, 2001; Elliott et al., Lancet 1994; 344: 1125-1127, 1994; Feldmann et al., Ann. Rev. Immunol. 19(1):163-196, 2001; Rankin et al., Br. J. Rheumatol. 2:334-342, 1995; Knight et al., Molecular Immunol. 30(16):1443-1453, 1993; Lorenz et al., J. Immunol. 156(4):1646-1653, 1996; Hinshaw et al., Circulatory Shock 30(3):279-292, 1990; Ordas et al., Clin. Pharmacol. Therapeutics 91(4):635-646, 2012; Feldman, Nature Reviews Immunol. 2(5):364-371, 2002; Taylor et al., Nature Reviews Rheumatol. 5(10):578-582, 2009; Garces et al., Annals Rheumatic Dis. 72(12):1947-1955, 2013; Palladino et al., Nature Rev. Drug Discovery 2(9):736-746, 2003; Sandborn et al., Inflammatory Bowel Diseases 5(2):119-133, 1999; Atzeni et al., Autoimmunity Reviews 12(7):703-708, 2013; Maini et al., Immunol. Rev. 144(1):195-223, 1995; Wanner et al., Shock 11(6):391-395, 1999; and U.S. Pat. Nos. 6,090,382; 6,258,562; and 6,509,015).

In certain embodiments, the anti-TNFα agent can include or is golimumab (Golimumab™), adalimumab (Humira™), infliximab (Remicade™), CDP571, CDP 870, or certolizumab pegol (Cimzia™). In certain embodiments, the anti-TNFα agent can be a TNFα inhibitor biosimilar. Examples of approved and late-phase TNFα inhibitor biosimilars include, but are not limited to, infliximab biosimilars such as Flixabi™ (SB2) from Samsung Bioepis, Inflectra® (CT-P13) from Celltrion/Pfizer, GS071 from Aprogen, Remsima™, PF-06438179 from Pfizer/Sandoz, NI-071 from Nichi-Iko Pharmaceutical Co., and ABP 710 from Amgen; adalimumab biosimilars such as Amgevita® (ABP 501) from Amgen and Exemptia™ from Zydus Cadila, BMO-2 or MYL-1401-A from Biocon/Mylan, CHS-1420 from Coherus, FKB327 from Kyowa Kirin, and BI 695501 from Boehringer Ingelheim; Solymbic®, SB5 from Samsung Bioepis, GP-2017 from Sandoz, ONS-3010 from Oncobiologics, M923 from Momenta, PF-06410293 from Pfizer, and etanercept biosimilars such as Erelzi™ from Sandoz/Novartis, Brenzys™ (SB4) from Samsung Bioepis, GP2015 from Sandoz, TuNEX® from Mycenax, LBEC0101 from LG Life, and CHS-0214 from Coherus.

In some embodiments of any of the methods described herein, the anti-TNFα agent is selected from the group consisting of: adalimumab, certolizumab, etanercept, golimumab, infliximabm, CDP571, and CDP 870.

In some embodiments, any of the antibodies or antigen-binding fragments described herein has a dissociation constant (K_{D}) of less than 1×10⁻⁵ M (e.g., less than 0.5×10⁻⁵ M, less than 1×10⁻⁶ M, less than 0.5×10⁻⁶ M, less than 1×10⁻⁷ M, less than 0.5×10⁻⁷ M, less than 1×10⁻⁸ M, less than 0.5×10⁻⁸ M, less than 1×10⁻⁹ M, less than 0.5×10⁻⁹ M, less than 1×10⁻¹⁰ M, less than 0.5×10⁻¹⁰ M, less than 1×10⁻¹¹ M, less than 0.5×10⁻¹¹ M, or less than 1×10⁻¹² M), e.g., as measured in phosphate buffered saline using surface plasmon resonance (SPR).

In some embodiments, any of the antibodies or antigen-binding fragments described herein has a K_{D} of about 1×10⁻¹² M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, about 1×10⁻¹⁰ M, about 0.5×10⁻¹⁰ M, about 1×10⁻¹¹ M, or about 0.5×10⁻¹¹ M (inclusive); about 0.5×10⁻¹¹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, about 1×10⁻¹⁰ M, about 0.5×10⁻¹⁰ M, or about 1×10⁻¹¹ M (inclusive); about 1×10⁻¹¹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, about 1×10⁻¹⁰ M, or about 0.5×10⁻¹⁰ M (inclusive); about 0.5×10⁻¹⁰ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, or about 1×10⁻¹⁰ M (inclusive); about 1×10⁻¹⁰ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, or about 0.5×10⁻⁹ M (inclusive); about 0.5×10⁻⁹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, or about 1×10⁻⁹ M (inclusive); about 1×10⁻⁹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, or about 0.5×10⁻⁸ M (inclusive); about 0.5×10⁻⁸ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, or about 1×10⁻⁸ M (inclusive); about 1×10⁻⁸ M to about 1×10⁻⁵ M, about 0.5×10⁻⁵ M, about 1×10⁻⁶ M, about 0.5×10⁻⁶ M, about 1×10⁻⁷ M, or about 0.5×10⁻⁷ M (inclusive); about 0.5×10⁻⁷ M to about 1×10⁻⁵ M, about 0.5×10⁻⁵ M, about 1×10⁻⁶ M, about 0.5×10⁻⁶ M, or about 1×10⁻⁷ M (inclusive); about 1×10⁻⁷ M to about 1×10⁻⁵ M, about 0.5×10⁻⁵ M, about 1×10⁻⁶ M, or about 0.5×10⁻⁶ M (inclusive); about 0.5×10⁻⁶ M to about 1×10⁻⁵ M, about 0.5×10⁻⁵ M, or about 1×10⁻⁶ M (inclusive); about 1×10⁻⁶ M to about 1×10⁻⁵ M or about 0.5×10⁻⁵ M (inclusive); or about 0.5×10⁻⁵ M to about 1×10⁻⁵ M (inclusive), e.g., as measured in phosphate buffered saline using surface plasmon resonance (SPR).

In some embodiments, any of the antibodies or antigen-binding fragments described herein has a K_{off} of about 1×10⁻⁶ s⁻¹ to about 1×10⁻³ s⁻¹, about 0.5×10⁻³ s⁻¹, about 1×10⁻⁴ s⁻¹, about 0.5×10⁻⁴ s⁻¹, about 1×10⁻⁵ s⁻¹, or about 0.5×10⁻⁵ s⁻¹ (inclusive); about 0.5×10⁻⁵ s⁻¹ to about 1×10⁻³ s⁻¹, about 0.5×10⁻³ s⁻¹, about 1×10⁻⁴ s⁻¹, about 0.5×10⁻⁴ s⁻¹, or about 1×10⁻⁵ s⁻¹ (inclusive); about 1×10⁻⁵ s⁻¹ to about 1×10⁻³ s⁻¹, about 0.5×10⁻³ s⁻¹, about 1×10⁻⁴ s⁻¹, or about 0.5×10⁻⁴ s⁻¹ (inclusive); about 0.5×10⁻⁴ s⁻¹ to about 1×10⁻³ s⁻¹, about 0.5×10⁻³ s⁻¹, or about 1×10⁻⁴ s⁻¹(inclusive); about 1×10⁻⁴ s⁻¹ to about 1×10⁻³ s⁻¹, or about 0.5×10⁻³ s⁻¹ (inclusive); or about 0.5×10⁻⁵ s⁻¹ to about 1×10⁻³ s⁻¹ (inclusive), e.g., as measured in phosphate buffered saline using surface plasmon resonance (SPR).

In some embodiments, any of the antibodies or antigen-binding fragments described herein has a Kₒₙ of about 1×10² M⁻¹s⁻¹ to about 1×10⁶ M⁻¹s⁻¹, about 0.5×10⁶ M⁻¹s⁻¹, about 1×10⁵ M⁻¹s⁻¹, about 0.5×10⁵ M⁻¹s⁻¹, about 1×10⁴ M⁻¹s⁻¹, about 0.5×10⁴ M⁻¹s⁻¹, about 1×10³ M⁻¹s⁻¹, or about 0.5×10³ M⁻¹s⁻¹ (inclusive); about 0.5×10³ M⁻¹s⁻¹ to about 1×10⁶ M⁻¹s⁻¹, about 0.5×10⁶ M⁻¹s⁻¹, about 1×10⁵ M⁻¹s⁻¹, about 0.5×10⁵ M⁻¹s⁻¹, about 1×10⁴ M⁻¹s⁻¹, about 0.5×10⁴ M⁻¹s⁻¹, or about 1×10³ M⁻¹s⁻¹ (inclusive); about 1×10³ M⁻¹s⁻¹ to about 1×10⁶ M⁻¹s⁻¹, about 0.5×10⁶ M⁻¹s⁻¹, about 1×10⁵ M⁻¹s⁻¹, about 0.5×10⁵ M⁻¹s⁻¹, about 1×10⁴ M⁻¹s⁻¹, or about 0.5×10⁴ M⁻¹s⁻¹ (inclusive); about 0.5×10⁴ M⁻¹s⁻¹ to about 1×10⁶ M⁻¹s⁻¹, about 0.5×10⁶ M⁻¹s⁻¹, about 1×10⁵ M⁻¹s⁻¹, about 0.5×10⁵ M⁻¹s⁻¹, or about 1×10⁴ M⁻¹s¹ (inclusive); about 1×10⁴ M⁻¹s⁻¹ to about 1×10⁶ M⁻¹s⁻¹, about 0.5×10⁶ M⁻¹s⁻¹, about 1×10⁵ M⁻¹s⁻¹, or about 0.5×10⁵ M⁻¹s⁻¹ (inclusive); about 0.5×10⁵ M⁻¹s⁻¹ to about 1×10⁶ M⁻¹s⁻¹, about 0.5×10⁶ M⁻¹s⁻¹, or about 1×10⁵ M⁻¹s⁻¹ (inclusive); about 1×10⁵ M⁻¹s⁻¹ to about 1×10⁶ M⁻¹s⁻¹, or about 0.5×10⁶ M⁻¹s⁻¹ (inclusive); or about 0.5×10⁶ M⁻¹s⁻¹ to about 1×10⁶ M⁻¹s⁻¹ (inclusive), e.g., as measured in phosphate buffered saline using surface plasmon resonance (SPR).

### Fusion Proteins

In some embodiments, the anti-TNFα agent is a fusion protein (e.g., an extracellular domain of a TNFR fused to a partner peptide, e.g., an Fc region of an immunoglobulin, e.g., human IgG) (see, e.g., Deeg et al., Leukemia 16(2):162, 2002; Peppel et al., J. Exp. Med. 174(6):1483-1489, 1991) or a soluble TNFR (e.g., TNFR1 or TNFR2) that binds specifically to TNFα. In some embodiments, the anti-TNFα agent includes or is a soluble TNFα receptor (e.g., Bjornberg et al., Lymphokine Cytokine Res. 13(3):203-211, 1994; Kozak et al., Am. J. Physiol. Reg. Integrative Comparative Physiol. 269(1):R23-R29, 1995; Tsao et al., Eur Respir J. 14(3):490-495, 1999; Watt et al., J Leukoc Biol. 66(6):1005-1013, 1999; Mohler et al., J. Immunol. 151(3):1548-1561, 1993; Nophar et al., EMBO J. 9(10):3269, 1990; Piguet et al., Eur. Respiratory J. 7(3):515-518, 1994; and Gray et al., Proc. Natl. Acad. Sci. U.S.A. 87(19):7380-7384, 1990). In some embodiments, the anti-TNFα agent includes or is etanercept (Enbrel™) (see, e.g., WO 91/03553 and WO 09/406,476, incorporated by reference herein). In some embodiments, the anti-TNFα agent inhibitor includes or is r-TBP-I (e.g., Gradstein et al., J. Acquir. Immune Defic. Syndr. 26(2): 111-117, 2001).

### Inhibitory Nucleic Acids

Inhibitory nucleic acids that can decrease the expression of AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA expression in a mammalian cell include antisense nucleic acid molecules, i.e., nucleic acid molecules whose nucleotide sequence is fully or partially complementary to all or part of a AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA (e.g., fully or partially complementary to all or a part of any one of the sequences presented in Table E).

TABLE E

| | mRNA GenBank |
|---|---|
| Human gene | accession number(s) |
| Tumor necrosis factor | NM_000594 |
| (TNF, a.k.a. TNF-alpha) | |
| TNF receptor superfamily member 1A | NM_001065 |
| (TNFRSF1A) (a.k.a. TNFR1) | NM_001346091 |
| | NM_001346092 |
| TNF receptor superfamily member 1B | NM_001066 |
| (TNFRSF1B) (a.k.a. TNFR2) | XM_011542060 |
| | XM_011542063 |
| | XM_017002214 |
| | XM_017002215 |
| | XM_017002211 |
| TNFRSF1A associated via death domain | NM_003789 |
| (TRADD) | NM_001323552 |
| | XM_005256213 |
| | XM_017023815 |
| TNF receptor associated factor 2 (TRAF2) | NM_021138 |
| | XM_011518976 |
| | XM_011518977 |
| | XM_011518974 |
| JunD proto-oncogene, AP-1 transcription | NM_001286968 |
| factor subunit (JUND) | NM_005354 |
| Mitogen-activated protein kinase kinase | NM_005923 |
| kinase 5 (MAP3K5) (a.k.a. ASK1) | XM_017010875 |
| | XM_017010872 |
| | XM_017010873 |
| | XM_017010877 |
| | XM_017010874 |
| | XM_017010871 |
| | XM_017010870 |
| | XM_017010876 |
| | XM_011535839 |
| CD14 | NM_000591 |
| | NM_001040021 |
| | NM_001174104 |
| | NM_001174105 |
| Mitogen-activated protein kinase 3 | NM_001040056 |
| (MAPK3) (a.k.a. ERK1) | NM_001109891 |
| | NM_002746 |
| Mitogen-activated protein kinase 1 | NM_002745 |
| (MAPK1) (a.k.a. ERK2) | NM_138957 |
| Inhibitor of nuclear factor kappa B kinase | NM_001190720 |
| subunit beta (IKBKB) | NM_001242778 |
| | NM_001556 |
| | XM_005273491 |
| | XM_005273496 |
| | XM_005273493 |
| | XM_005273498 |
| | XM_011544518 |
| | XM_005273492 |
| | XM_005273490 |
| | XM_005273494 |
| | 12XM_017013396 |
| | XM_011544521 |
| | XM_011544522 |
| | XM_005273495 |
| | XM_011544517 |
| | XM_011544520 |
| | XM_011544519 |
| NFKB inhibitor alpha (NFKBIA) | NM_020529 |
| Interleukin 1 receptor associated kinase 1 | NM_001025242 |
| (IRAK1) | NM_001025243 |
| | NM_001569 |
| | XM_005274668 |
| Mitogen-activated protein kinase 8 | NM_001278547 |
| (MAPK8) (a.k.a. JNK) | NM_001278548 |
| | NM_001323302 |
| | NM_001323320 |
| | NM_001323321 |
| | NM_001323322 |
| | NM_001323323 |
| | NM_001323324 |
| | NM_001323325 |
| | NM_001323326 |
| | NM_001323327 |
| | NM_001323328 |
| | NM_001323329 |
| | NM_001323330 |
| | NM_001323331 |
| | NM_139046 |
| | NM_139049 |
| | XM_024448079 |
| | XM_024448080 |
| Lipopolysaccharide binding protein (LBP) | NM_004139 |
| Mitogen-activated protein kinase kinase 1 | NM_002755 |
| (MAP2K1) (a.k.a. MEK1) | XM_017022411 |
| | XM_011521783 |
| | XM_017022412 |
| | XM_017022413 |
| Mitogen-activated protein kinase kinase 2 | NM_030662 |
| (MAP2K2) (a.k.a. MEK2) | XM_006722799 |
| | XM_017026990 |
| | XM_017026989 |
| | XM_017026991 |
| Mitogen-activated protein kinase kinase 3 | NM_001316332 |
| (MAP2K3) (a.k.a. MEK3) | NM_002756 |
| | NM_145109 |
| | XM_017024859 |
| | XM_005256723 |
| | XM_017024857 |
| | XM_011523959 |
| | XM_017024858 |
| | XM_011523958 |
| Mitogen-activated protein kinase kinase 6 | NM_001330450 |
| (MAP2K6) (a.k.a. MEK6) | NM_002758 |
| | XM_005257516 |
| | XM_011525027 |
| | XM_011525026 |
| | XM_006721975 |
| Mitogen-activated protein kinase kinase | NM_005921 |
| kinase 1 (MAP3K1) (a.k.a. MEKK1) | XM_017009485 |
| | XM_017009484 |
| Mitogen-activated protein kinase kinase | NM_001330431 |
| kinase 3 (MAP3K3) (a.k.a. MEKK3) | NM_001363768 |
| | NM_002401 |
| | NM_203351 |
| | XM_005257378 |
| Mitogen-activated protein kinase kinase | NM_001291958 |
| kinase 4 (MAP3K4) (a.k.a. MEKK4) | NM_001301072 |
| | NM_001363582 |
| | NM_005922 |
| | NM_006724 |
| | XM_017010869 |
| Mitogen-activated protein kinase kinase | NM_001297609 |
| kinase 6 (MAP3K6) (a.k.a. MEKK6) | NM_004672 |
| | XM_017002771 |
| | XM_017002772 |
| Mitogen-activated protein kinase kinase | NM_003188 |
| kinase 7 (MAP3K7) (a.k.a. MEKK7) | NM_145331 |
| | NM_145332 |
| | NM_145333 |
| | XM_006715553 |
| | XM_017011226 |
| MAPK activated protein kinase 2 | NM_004759 |
| (MAPKAPK2) (a.k.a. MK2) | NM_032960 |
| | XM_005273353 |
| | XM_017002810 |
| MYD88, innate immune signal transduction | NM_001172566 |
| adaptor (MYD88) | NM_001172567 |
| | NM_001172568 |
| | NM_001172569 |
| | NM_001365876 |
| | NM_001365877 |
| | NM_002468 |
| Nuclear factor kappa B subunit 1 (NFKB1) | NM_001165412 |
| | NM_001319226 |
| | NM_003998 |
| | XM_024454069 |
| | XM_024454067 |
| | XM_011532006 |
| | XM_024454068 |
| Mitogen-activated protein kinase kinase | NM_003954 |
| kinase 14 (MAP3K14) (a.k.a. NIK) | XM_011525441 |
| Mitogen-activated protein kinase 14 | NM_001315 |
| (MAPK14) (a.k.a. p38) | NM_139012 |
| | NM_139013 |
| | NM_139014 |
| | XM_011514310 |
| | XM_017010300 |
| | XM_017010299 |
| | XM_017010301 |
| | XM_017010304 |
| | XM_017010303 |
| | XM_017010302 |
| | XM_006714998 |
| Eukaryotic translation initiation factor 2 | NM_001135651 |
| alpha kinase 2 (EIF2AK2) (a.k.a. PKR) | NM_001135652 |
| | NM_002759 |
| | XM_011532987 |
| | XM_017004503 |
| AKT serine/threonine kinase 1 (AKT1) | NM_001014431 |
| (a.k.a. RAC) | NM_001014432 |
| | NM_005163 |
| Zinc fingers and homeoboxes 2 (ZHX2) | NM_001362797 |
| (a.k.a. RAF) | NM_014943 |
| | XM_011516932 |
| | XM_005250836 |
| KRAS proto-oncogene, GTPase (KRAS) | NM_001369786 |
| | NM_001369787 |
| | NM_004985 |
| | NM_033360 |
| NRAS proto-oncogene, GTPase (NRAS) | NM_002524 |
| Receptor interacting serine/threonine kinase | NM_001317061 |
| 1 (RIPK1) (a.k.a. RIP) | NM_001354930 |
| | NM_001354931 |
| | NM_001354932 |
| | NM_001354933 |
| | NM_001354934 |
| | NM_003804 |
| | XM_017011405 |
| | XM_006715237 |
| | XM_017011403 |
| | XM_017011404 |
| TNF receptor associated factor 6 (TRAF6) | NM_004620 |
| | NM_145803 |
| | XM_017018220 |
| ZFP36 ring finger protein (ZFP36) (a.k.a. | NM_003407 |
| TTP) | |

An antisense nucleic acid molecule can be fully or partially complementary to all or part of a non-coding region of the coding strand of a nucleotide sequence encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RTP, TNFα, TNFR1, TNFR2, TRADD, TRAF2, TRAF6, or TTP protein. Non-coding regions (5′ and 3′ untranslated regions) are the 5′ and 3′ sequences that flank the coding region in a gene and are not translated into amino acids.

Based upon the sequences disclosed herein, one of skill in the art can easily choose and synthesize any of a number of appropriate antisense nucleic acids to target a nucleic acid encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP protein described herein. Antisense nucleic acids targeting a nucleic acid encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP protein can be designed using the software available at the Integrated DNA Technologies website.

An antisense nucleic acid can be, for example, about 5, 10, 15, 18, 20, 22, 24, 25, 26, 28, 30, 32, 35, 36, 38, 40, 42, 44, 45, 46, 48, or 50 nucleotides or more in length. An antisense oligonucleotide can be constructed using enzymatic ligation reactions and chemical synthesis using procedures known in the art. For example, an antisense nucleic acid can be chemically synthesized using variously modified nucleotides or naturally occurring nucleotides designed to increase the physical stability of the duplex formed between the antisense and sense nucleic acids, e.g., phosphorothioate derivatives and acridine substituted nucleotides or to increase the biological stability of the molecules.

Examples of modified nucleotides which can be used to generate an antisense nucleic acid include 1-methylguanine, 1-methylinosine, 2,2-dimethylguanine, 2-methyladenine, 2-methylguanine, 3-methylcytosine, 2-methylthio-N6-isopentenyladenine, uracil-5-oxyacetic acid (v), wybutoxosine, pseudouracil, queosine, 2-thiocytosine, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

The antisense nucleic acid molecules described herein can be prepared in vitro and administered to a subject, e.g., a human subject. Alternatively, they can be generated in situ such that they hybridize with or bind to cellular mRNA and/or genomic DNA encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP protein to thereby inhibit expression, e.g., by inhibiting transcription and/or translation. The hybridization can be by conventional nucleotide complementarities to form a stable duplex, or, for example, in the case of an antisense nucleic acid molecule that binds to DNA duplexes, through specific interactions in the major groove of the double helix. The antisense nucleic acid molecules can be delivered to a mammalian cell using a vector (e.g., an adenovirus vector, a lentivirus, or a retrovirus).

An antisense nucleic acid can be an α-anomeric nucleic acid molecule. An α-anomeric nucleic acid molecule forms specific double-stranded hybrids with complementary RNA in which, contrary to the usual, β-units, the strands run parallel to each other (Gaultier et al., Nucleic Acids Res. 15:6625-6641, 1987). The antisense nucleic acid can also comprise a chimeric RNA-DNA analog (Inoue et al., FEBS Lett. 215:327-330, 1987) or a 2′-O-methylribonucleotide (Inoue et al., Nucleic Acids Res. 15:6131-6148, 1987).

Another example of an inhibitory nucleic acid is a ribozyme that has specificity for a nucleic acid encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA, e.g., specificity for any one of the sequences presented in Table E). Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach, Nature 334:585-591, 1988)) can be used to catalytically cleave mRNA transcripts to thereby inhibit translation of the protein encoded by the mRNA. An AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA can be used to select a catalytic RNA having a specific ribonuclease activity from a pool of RNA molecules. See, e.g., Bartel et al., Science 261:1411-1418, 1993.

Alternatively, a ribozyme having specificity for an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA can be designed based upon the nucleotide sequence of any of the AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA sequences disclosed herein (e.g., in Table E). For example, a derivative of a Tetrahymena L-19 IVS RNA can be constructed in which the nucleotide sequence of the active site is complementary to the nucleotide sequence to be cleaved in an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA (see, e.g., U.S. Pat. Nos. 4,987,071 and 5,116,742).

An inhibitory nucleic acid can also be a nucleic acid molecule that forms triple helical structures. For example, expression of an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP polypeptide can be inhibited by targeting nucleotide sequences complementary to the regulatory region of the gene encoding the AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP polypeptide (e.g., the promoter and/or enhancer, e.g., a sequence that is at least 1 kb, 2 kb, 3 kb, 4 kb, or 5 kb upstream of the transcription initiation start state) to form triple helical structures that prevent transcription of the gene in target cells. See generally Maher, Bioassays 14(12):807-15, 1992; Helene, Anticancer Drug Des. 6(6):569-84, 1991; and Helene, Ann. N.Y. Acad. Sci. 660:27-36, 1992.

In various embodiments, inhibitory nucleic acids can be modified at the sugar moiety, the base moiety, or phosphate backbone to improve, e.g., the solubility, stability, or hybridization, of the molecule. For example, the deoxyribose phosphate backbone of the nucleic acids can be modified to generate peptide nucleic acids (see, e.g., Hyrup et al., Bioorganic Medicinal Chem. 4(1):5-23, 1996). Peptide nucleic acids (PNAs) are nucleic acid mimics, e.g., DNA mimics, in which the deoxyribose phosphate backbone is replaced by a pseudopeptide backbone and only the four natural nucleobases are retained. The neutral backbone of PNAs allows for specific hybridization to RNA and DNA under conditions of low ionic strength. PNA oligomers can be synthesized using standard solid phase peptide synthesis protocols (see, e.g., Perry-O'Keefe et al., Proc. Natl. Acad. Sci. U.S.A. 93:14670-675, 1996). PNAs can be used as antisense or antigene agents for sequence-specific modulation of gene expression by, e.g., inducing transcription or translation arrest or inhibiting replication.

### Small Molecules

In some embodiments, the anti-TNFα agent is a small molecule. In some embodiments, the small molecule is a tumor necrosis factor-converting enzyme (TACE) inhibitor (e.g., Moss et al., Nature Clinical Practice Rheumatology 4: 300-309, 2008). In some embodiments, the anti-TNFα agent is C87 (Ma et al., J. Biol. Chem. 289(18):12457-66, 2014). In some embodiments, the small molecule is LMP-420 (e.g., Haraguchi et al., AIDS Res. Ther. 3:8, 2006). In some embodiments, the TACE inhibitor is TMI-005 and BMS-561392. Additional examples of small molecule inhibitors are described in, e.g., He et al., Science 310(5750):1022-1025, 2005.

In some examples, the anti-TNFα agent is a small molecule that inhibits the activity of one of AP-1, ASK1, IKK, JNK, MAPK, MEKK 1/4, MEKK4/7, MEKK 3/6, NIK, TRADD, RIP, NF-κB, and TRADD in a cell (e.g., in a cell obtained from a subject, a mammalian cell).

In some examples, the anti-TNFα agent is a small molecule that inhibits the activity of one of CD14, MyD88 (see, e.g., Olson et al., Scientific Reports 5:14246, 2015), ras (e.g., Baker et al., Nature 497:577-578, 2013), raf (e.g., vemurafenib (PLX4032, RG7204), sorafenib tosylate, PLX-4720, dabrafenib (GSK2118436), GDC-0879, RAF265 (CHIR-265), AZ 628, NVP-BHG712, SB590885, ZM 336372, sorafenib, GW5074, TAK-632, CEP-32496, encorafenib (LGX818), CCT196969, LY3009120, RO5126766 (CH5126766), PLX7904, and MLN2480).

In some examples, the anti-TNFα agent TNFα inhibitor is a small molecule that inhibits the activity of one of MK2 (PF 3644022 and PHA 767491), INK (e.g., AEG 3482, BI 78D3, CEP 1347, c-JUN peptide, IQ 1S, JIP-1 (153-163), SP600125, SU 3327, and TCS JNK6o), c-jun (e.g., AEG 3482, BI 78D3, CEP 1347, c-JUN peptide, IQ 1S, JIP-1 (153-163), SP600125, SU 3327, and TCS JNK6o), MEK3/6 (e.g., Akinleye et al., J. Hematol. Oncol. 6:27, 2013), p38 (e.g., AL 8697, AMG 548, BIRB 796, CMPD-1, DBM 1285 dihydrochloride, EO 1428, JX 401, ML 3403, Org 48762-0, PH 797804, RWJ 67657, SB 202190, SB 203580, SB 239063, SB 706504, SCIO 469, SKF 86002, SX 011, TA 01, TA 02, TAK 715, VX 702, and VX 745), PKR (e.g., 2-aminopurine or CAS 608512-97-6), TTP (e.g., CAS 329907-28-0), MEK1/2 (e.g., Facciorusso et al., Expert Review Gastroentrol. Hepatol. 9:993-1003, 2015), ERK1/2 (e.g., Mandal et al., Oncogene 35:2547-2561, 2016), NIK (e.g., Mortier et al., Bioorg. Med. Chem. Lett. 20:4515-4520, 2010), IKK (e.g., Reilly et al., Nature Med. 19:313-321, 2013), IκB (e.g., Suzuki et al., Expert. Opin. Invest. Drugs 20:395-405, 2011), NF-κB (e.g., Gupta et al., Biochim. Biophys. Acta 1799(10-12):775-787, 2010), rac (e.g., U.S. Pat. No. 9,278,956), MEK4/7, IRAK (Chaudhary et al., J. Med. Chem. 58(1):96-110, 2015), LBP (see, e.g., U.S. Pat. No. 5,705,398), and TRAF6 (e.g., 3-[(2,5-Dimethylphenyl)amino]-1-phenyl-2-propen-1-one).

In some embodiments of any of the methods described herein, the inhibitory nucleic acid can be about 10 nucleotides to about 50 nucleotides (e.g., about 10 nucleotides to about 45 nucleotides, about 10 nucleotides to about 40 nucleotides, about 10 nucleotides to about 35 nucleotides, about 10 nucleotides to about 30 nucleotides, about 10 nucleotides to about 28 nucleotides, about 10 nucleotides to about 26 nucleotides, about 10 nucleotides to about 25 nucleotides, about 10 nucleotides to about 24 nucleotides, about 10 nucleotides to about 22 nucleotides, about 10 nucleotides to about 20 nucleotides, about 10 nucleotides to about 18 nucleotides, about 10 nucleotides to about 16 nucleotides, about 10 nucleotides to about 14 nucleotides, about 10 nucleotides to about 12 nucleotides, about 12 nucleotides to about 50 nucleotides, about 12 nucleotides to about 45 nucleotides, about 12 nucleotides to about 40 nucleotides, about 12 nucleotides to about 35 nucleotides, about 12 nucleotides to about 30 nucleotides, about 12 nucleotides to about 28 nucleotides, about 12 nucleotides to about 26 nucleotides, about 12 nucleotides to about 25 nucleotides, about 12 nucleotides to about 24 nucleotides, about 12 nucleotides to about 22 nucleotides, about 12 nucleotides to about 20 nucleotides, about 12 nucleotides to about 18 nucleotides, about 12 nucleotides to about 16 nucleotides, about 12 nucleotides to about 14 nucleotides, about 15 nucleotides to about 50 nucleotides, about 15 nucleotides to about 45 nucleotides, about 15 nucleotides to about 40 nucleotides, about 15 nucleotides to about 35 nucleotides, about 15 nucleotides to about 30 nucleotides, about 15 nucleotides to about 28 nucleotides, about 15 nucleotides to about 26 nucleotides, about 15 nucleotides to about 25 nucleotides, about 15 nucleotides to about 24 nucleotides, about 15 nucleotides to about 22 nucleotides, about 15 nucleotides to about 20 nucleotides, about 15 nucleotides to about 18 nucleotides, about 15 nucleotides to about 16 nucleotides, about 16 nucleotides to about 50 nucleotides, about 16 nucleotides to about 45 nucleotides, about 16 nucleotides to about 40 nucleotides, about 16 nucleotides to about 35 nucleotides, about 16 nucleotides to about 30 nucleotides, about 16 nucleotides to about 28 nucleotides, about 16 nucleotides to about 26 nucleotides, about 16 nucleotides to about 25 nucleotides, about 16 nucleotides to about 24 nucleotides, about 16 nucleotides to about 22 nucleotides, about 16 nucleotides to about 20 nucleotides, about 16 nucleotides to about 18 nucleotides, about 18 nucleotides to about 20 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 28 nucleotides, about 20 nucleotides to about 26 nucleotides, about 20 nucleotides to about 25 nucleotides, about 20 nucleotides to about 24 nucleotides, about 20 nucleotides to about 22 nucleotides, about 24 nucleotides to about 50 nucleotides, about 24 nucleotides to about 45 nucleotides, about 24 nucleotides to about 40 nucleotides, about 24 nucleotides to about 35 nucleotides, about 24 nucleotides to about 30 nucleotides, about 24 nucleotides to about 28 nucleotides, about 24 nucleotides to about 26 nucleotides, about 24 nucleotides to about 25 nucleotides, about 26 nucleotides to about 50 nucleotides, about 26 nucleotides to about 45 nucleotides, about 26 nucleotides to about 40 nucleotides, about 26 nucleotides to about 35 nucleotides, about 26 nucleotides to about 30 nucleotides, about 26 nucleotides to about 28 nucleotides, about 28 nucleotides to about 50 nucleotides, about 28 nucleotides to about 45 nucleotides, about 28 nucleotides to about 40 nucleotides, about 28 nucleotides to about 35 nucleotides, about 28 nucleotides to about 30 nucleotides, about 30 nucleotides to about 50 nucleotides, about 30 nucleotides to about 45 nucleotides, about 30 nucleotides to about 40 nucleotides, about 30 nucleotides to about 38 nucleotides, about 30 nucleotides to about 36 nucleotides, about 30 nucleotides to about 34 nucleotides, about 30 nucleotides to about 32 nucleotides, about 32 nucleotides to about 50 nucleotides, about 32 nucleotides to about 45 nucleotides, about 32 nucleotides to about 40 nucleotides, about 32 nucleotides to about 35 nucleotides, about 35 nucleotides to about 50 nucleotides, about 35 nucleotides to about 45 nucleotides, about 35 nucleotides to about 40 nucleotides, about 40 nucleotides to about 50 nucleotides, about 40 nucleotides to about 45 nucleotides, about 42 nucleotides to about 50 nucleotides, about 42 nucleotides to about 45 nucleotides, or about 45 nucleotides to about 50 nucleotides) in length. One skilled in the art will appreciate that inhibitory nucleic acids may comprises at least one modified nucleic acid at either the 5′ or 3′ end of DNA or RNA.

In some embodiments, the inhibitory nucleic acid can be formulated in a liposome, a micelle (e.g., a mixed micelle), a nanoemulsion, or a microemulsion, a solid nanoparticle, or a nanoparticle (e.g., a nanoparticle including one or more synthetic polymers). Additional exemplary structural features of inhibitory nucleic acids and formulations of inhibitory nucleic acids are described in US 2016/0090598.

In some embodiments, the inhibitory nucleic acid (e.g., any of the inhibitory nucleic acid described herein) can include a sterile saline solution (e.g., phosphate-buffered saline (PBS)). In some embodiments, the inhibitory nucleic acid (e.g., any of the inhibitory nucleic acid described herein) can include a tissue-specific delivery molecule (e.g., a tissue-specific antibody).

Compound Preparation and Biological Assays

As can be appreciated by the skilled artisan, methods of synthesizing the compounds of the formulae herein will be evident to those of ordinary skill in the art. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and RGM. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

### PREPARATIVE EXAMPLES

The following abbreviations have the indicated meanings:

ACN=acetonitrile
AcOH=acetic acid

### AIBN=Azodiisobutyronitrile

9-BBN=9-borabicyclo[3.3.1]nonane
Boc2O=Di-tert-butyl dicarbonate
(Bpin)2=4,4,4′,4′,5,5,5′,5′-octamethyl-2,2′-bi(1,3,2-dioxaborolane)
CAN=Diammonium cerium(IV) nitrate

### CCl4=Perchloromethane

### CHCl3=Chloroform

ClSO2OH=Chlorosulfonic acid

### Conc.=Concentrated

Cs2CO3=Cesium carbonate
DAST=diethylaminosulfur trifluoride
DBU=1,8-diazabicycloundec-7-ene
DCM=dichloromethane
DEA=diethylamine
DBDMH=1,3-dibromo-5,5-dimethylhydantoin

### DMF=N,N-dimethylformamide

DMSO=dimethyl sulfoxide

### DIEA=N,N-diisopropylethylamine

EtOH=ethanol
FA=formic acid

### HCHO=Formaldehyde

Hex=hexane
HPLC=high performance liquid chromatography
IPA=propan-2-ol
LC-MS=liquid chromatography-mass spectrometry
LDA=Lithium diisopropylamide
Me=methyl
MeOH=methanol
MSA=Methanesulfonic acid
Mts=2,4,6-trimethylbenzene sulfonyl
NaBH3CN=Sodium cyanoborohydride
NaSH=Sodium hydrosulfide

### NBS=N-bromosuccinimide

n-BuLi=n-Butyllithium
NMR=nuclear magnetic resonance
NMO=4-Methylmorpholine 4-oxide
PCl₅=Phosphorus pentachloride
Pd₂(dba)₃=tris(dibenzylideneacetone)dipalladium
Pd(dppf)Cl₂=dichloro[1,1′-bis(diphenylphosphino)ferrocene]palladium
Pd(PPh₃)₂Cl₂=Bis(triphenylphosphine)palladium(II) chloride
Ph=phenyl
PMB=p-methoxybenzyl
PPh₃Cl₂=dichlorotriphenylphosphorane
RuPhos=2-Dicyclohexylphosphino-2′,6′-diisopropoxy-1,1′-biphenyl
Rt=Retention time
RT=room temperature
SFC=supercritical fluid chromatogram
Sat.=saturated
SPhos=2-Dicyclohexylphosphino-2′,6′-dimethoxybiphenyl
TBAF=tetra-n-butylammonium fluoride
TBS=tert-butyldimethylsilyl
TBDPSCl=tert-butyldiphenylsilyl chloride
TBSCl=tert-butyldimethylsilyl chloride
t-BuOK=Potassium t-butoxide
t-BuONO=tert-Butyl nitrite
TEA=triethylamine
TFA=trifluoroacetic acid
THF=tetrahydrofuran
TLC=thin layer chromatography
UV=ultraviolet
X-phos=2-(Dicyclohexylphosphino)-2′,4′,6′-triisopropylbiphenyl

### General

The progress of reactions was often monitored by TLC or LC-MS. The identity of the products was often confirmed by LC-MS. The LC-MS was recorded using one of the following methods.

Method A: Shim-pack XR-ODS, C18, 3×50 mm, 2.5 um column, 1.0 uL injection, 1.5 mL/min flow rate, 90-900 amu scan range, 190-400 nm UV range, 5-100% (1.1 min), 100% (0.6 min) gradient with ACN (0.05% TFA) and water (0.05% TFA), 2 minute total run time.

Method B: Kinetex EVO, C18, 3×50 mm, 2.2 um column, 1.0 uL injection, 1.5 mL/min flow rate, 90-900 amu scan range, 190-400 nm UV range, 10-95% (1.1 min), 95% (0.6 min) gradient with ACN and water (0.5% NH4HCO3), 2 minute total run time.

Method C: Shim-pack XR-ODS, C18, 3×50 mm, 2.5 um column, 1.0 uL injection, 1.5 mL/min flow rate, 90-900 amu scan range, 190-400 nm UV range, 5-100% (2.1 min), 100% (0.6 min) gradient with ACN (0.05% TFA) and water (0.05% TFA), 3 minute total run time.

Method D: Kinetex EVO, C18, 3×50 mm, 2.2 um column, 1.0 uL injection, 1.5 mL/min flow rate, 90-900 amu scan range, 190-400 nm UV range, 10-95% (2.1 min), 95% (0.6 min) gradient with ACN and water (0.5% NH4HCO3), 3 minute total run time.

The final targets were purified by Prep-HPLC. The Prep-HPLC was carried out using the following method.

Method E: Pre-HPLC: Column, XBridge Shield RP18 OBD (19×250 mm, 10 um); mobile phase, Water (10 mmol/L NH4HCO3) and ACN, UV detection 254/210 nm.

NMR was recorded on BRUKER NMR 300.03 MHz, DUL-C-H, ULTRASHIELDTM300, AVANCE II 300 B-ACSTM120 or BRUKER NMR 400.13 MHz, BBFO, ULTRASHIELDTM400, AVANCE III 400, B-ACSTM120.

Racemic compounds of this invention can be resolved to give individual enantiomers using a variety of known methods. For example, chiral stationary phases can be used and the elution conditions can include normal phase or super-critical fluid with or without acidic or basic additives. Enantiomerically pure acids or bases can be used to form diastereomeric salts with the racemic compounds whereby pure enantiomers can be obtained by fractional crystallization. The racemates can also be derivatized with enantiomerically pure auxiliary reagents to form diastereomeric mixtures that can be separated. The auxiliary is then removed to give pure enantiomers.

Scheme of final targets: Schemes 1-6 illustrate several conditions used for coupling of acid 1 and sulfonimidoylamide 2 to afford acyl sulfonimidoylamide 3. As used in the schemes, rings “A” and “B” may be substituted as disclosed herein.

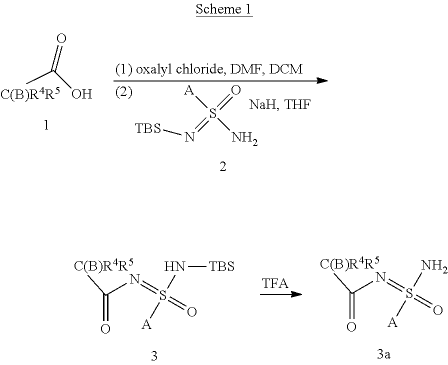

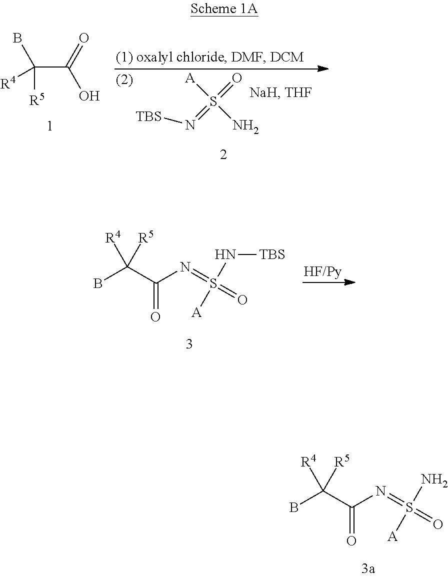

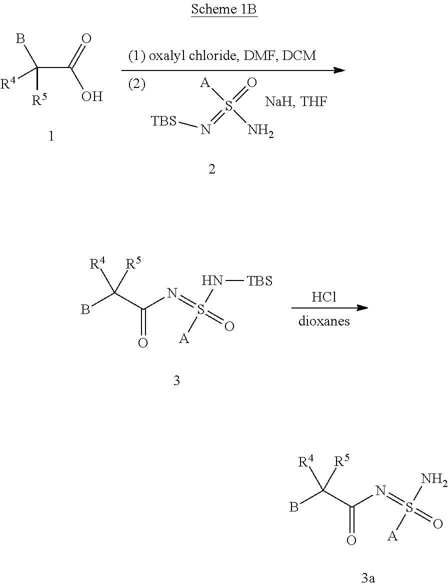

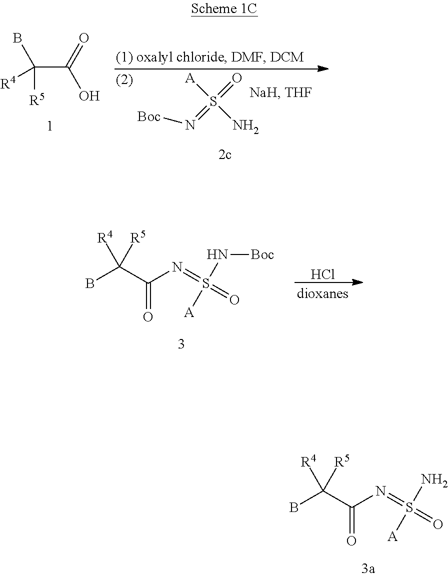

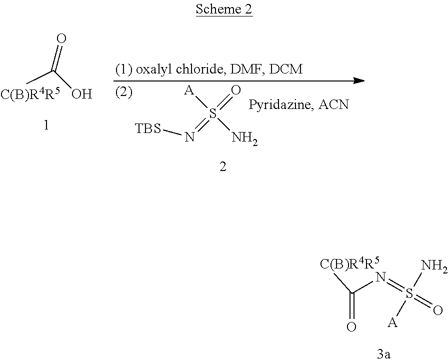

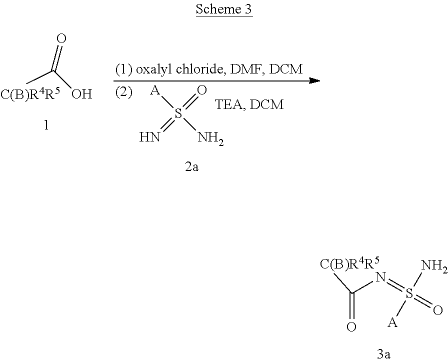

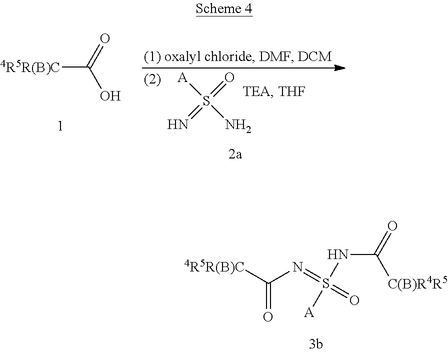

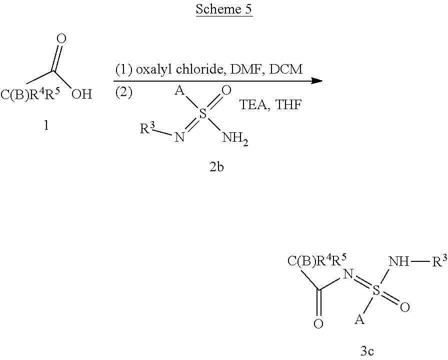

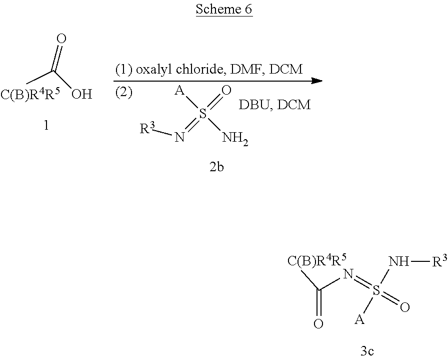

Scheme of final targets: Schemes I-IV illustrate several conditions used for coupling of acid 1′ and sulfonimidoylamide 2′ to afford acyl sulfonimidoylamide 3′

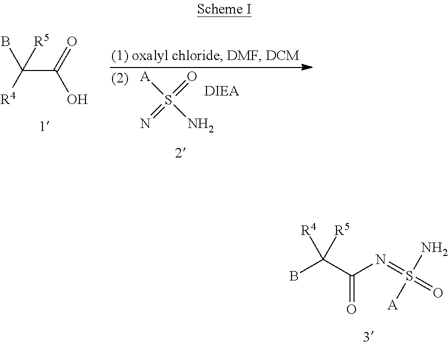

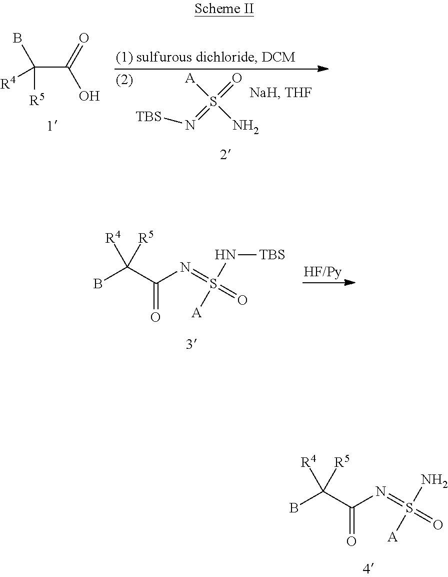

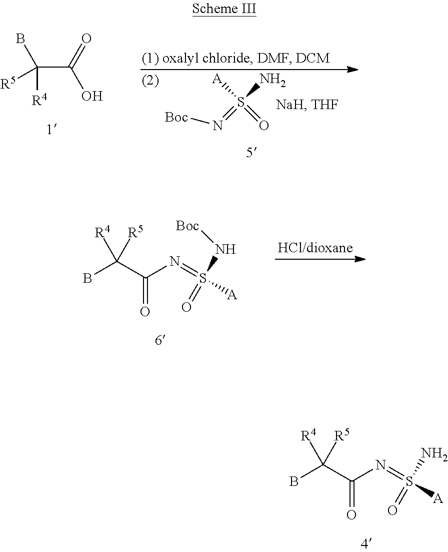

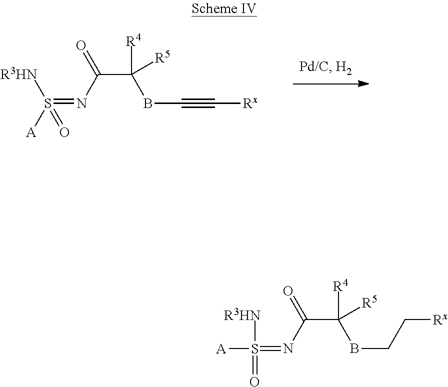

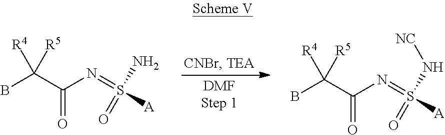

Scheme of final targets: Scheme below illustrated conditions used for coupling of acid and sulfonimidoylamide to afford acyl sulfonimidoylamide.

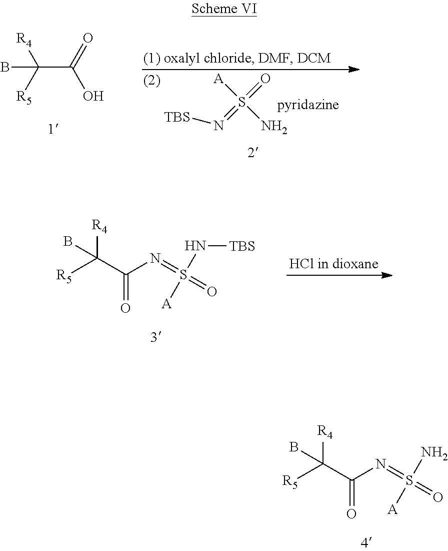

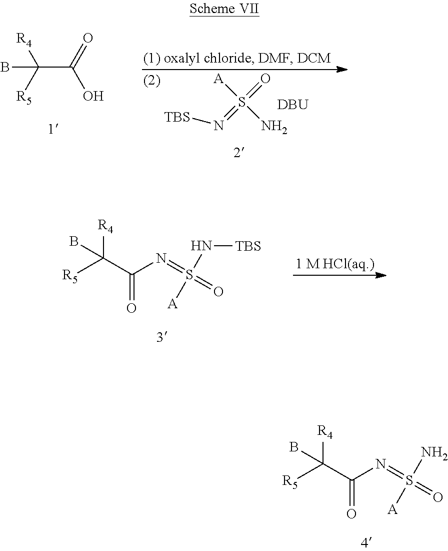

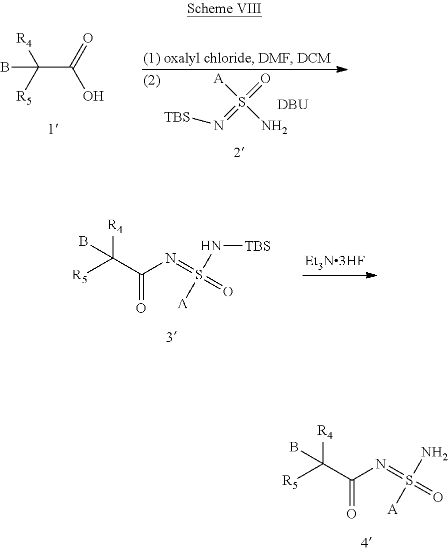

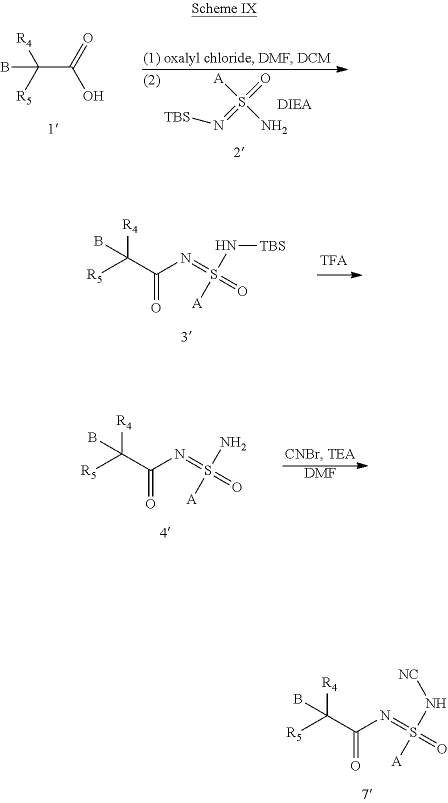

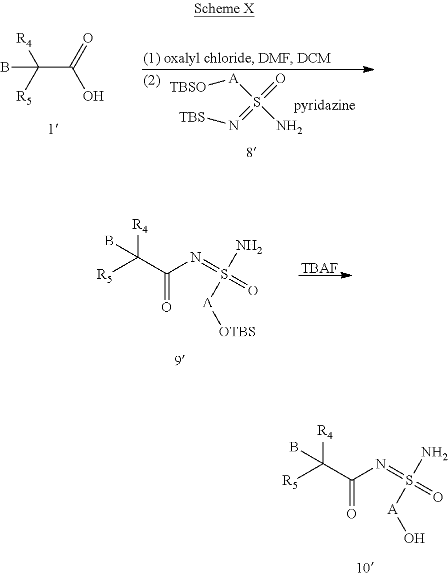

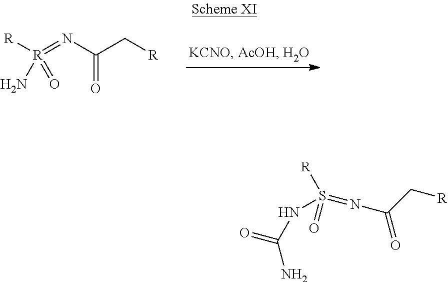

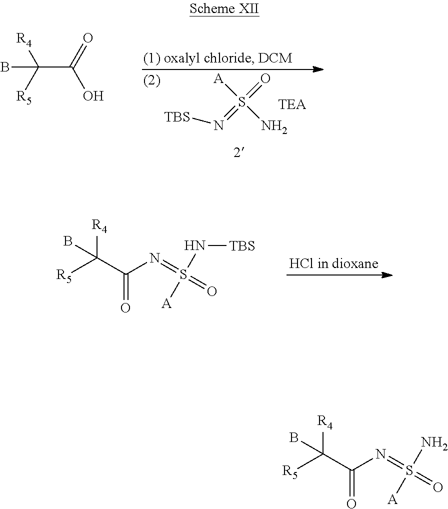

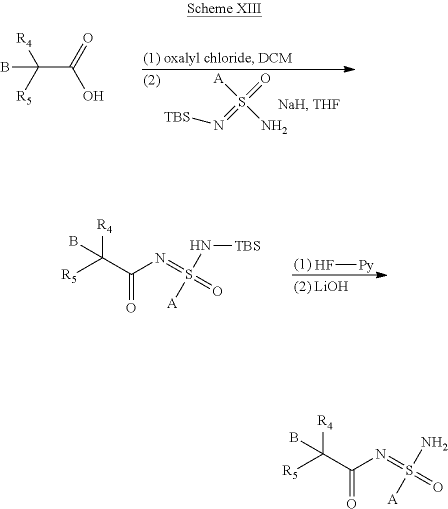

Schemes of Sulfonimidoylamide Intermediates: Schemes 7-12 illustrate the preparation of sulfonimidoylamide intermediates.

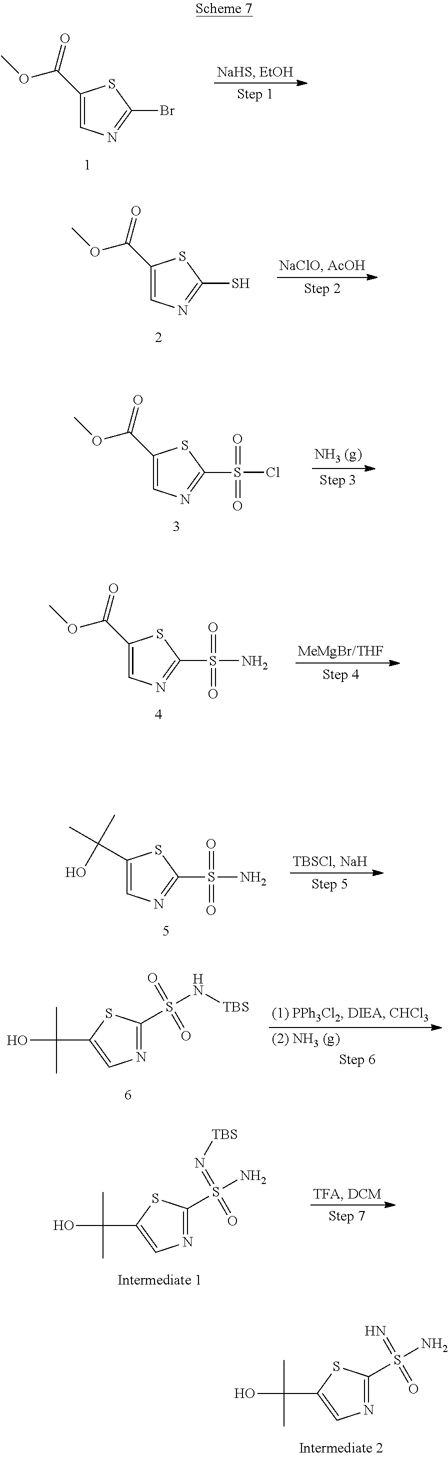

### Intermediate 1

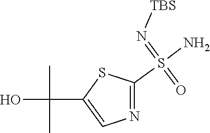

### N′-(tert-butyldimethylsilyl)-5-(2-hydroxypropan-2-yl)thiazole-2-sulfonimidamide

### Step 1: Methyl 2-mercaptothiazole-5-carboxylate

Into a 2000-mL round-bottom flask was placed methyl 2-bromothiazole-5-carboxylate (100 g, 450 mmol), EtOH (1000 mL), and sodium hydrogensulfide (50 g, 890 mmol). The resulting solution was stirred for 2 h at 80° C. and then was cooled to 0° C. with a water/ice bath. The pH value of the solution was adjusted to 3 with hydrogen chloride (1 N). The solids were collected by filtration. This resulted in 63.2 g (80%) of the title compound as a light yellow solid. MS-ESI: 176.0 (M+1).

### Step 2: Methyl 2-(chlorosulfonyl)thiazole-5-carboxylate

Into a 1000-mL round-bottom flask was placed methyl 2-mercaptothiazole-5-carboxylate (30 g, 170 mmol) and acetic acid (300 mL). This was followed by the addition of sodium hypochlorite (300 mL, 8%-10% wt.) in portions at 0° C. The resulting solution was stirred for 2 h at RT and then was diluted with 500 mL of water. The solution was extracted with 3×300 mL of DCM; and the combined organic layers were washed with 2×300 mL of brine, and dried over anhydrous Na₂SO₄. The crude product as a yellow solution in DCM was used in the next step.

### Step 3: Methyl 2-sulfamoylthiazole-5-carboxylate

Into a 2000-mL round-bottom flask was placed methyl 2-(chlorosulfonyl)thiazole-5-carboxylate as a crude solution in DCM (900 mL). To the solution was introduced NH₃ (g) below 0° C. for 20 minutes. The resulting solution was stirred for 1 h at RT and was then concentrated under vacuum. The residue was applied onto a silica gel column and eluted with ethyl acetate/petroleum ether (1:5 to 1:3). This resulted in 23 g (75%, 2 steps) of the title compound as a white solid. MS-ESI: 223.0 (M+1).

### Step 4: 5-(2-Hydroxypropan-2-yl)thiazole-2-sulfonamide

Into a 500-mL round-bottom flask purged with and maintained under nitrogen was placed a solution of methyl 2-sulfamoylthiazole-5-carboxylate (15 g, 67.5 mmol) in THF (150 mL). This was followed by the addition of MeMgBr/THF (3 M, 90 mL) dropwise with stirring at 0° C. The resulting solution was stirred for 14 h at RT and then was quenched by the addition of 100 mL of NH₄Cl (sat.). The resulting solution was extracted with 3×150 mL of DCM; the organic layers were combined, dried over anhydrous Na₂SO₄, and concentrated under vacuum. The residue was applied onto a silica gel column and eluted with ethyl acetate/petroleum ether (1:5 to 1:3). This resulted in 11.5 g (78%) of the title compound as a white solid. MS-ESI: 223.0 (M+1), 221.0 (M−1) in positive and negative ion mode, respectively.

### Step 5: N-(tert-butyldimethylsilyl)-5-(2-hydroxypropan-2-yl)thiazole-2-sulfonamide

Into a 250-mL 3-necked round-bottom flask purged with and maintained under nitrogen was placed a solution of 5-(2-hydroxypropan-2-yl)thiazole-2-sulfonamide (5 g, 22.5 mmol) in THF (100 mL). Then to the above was added NaH (60% wt, 1.8 g, 45.0 mmol) in portions in an ice/water bath. After stirring for 20 minutes in an ice/water bath, this was followed by the addition of a solution of TBSCl (4.1 g, 27.2 mmol) in THF (10 mL) dropwise with stirring over 2 min at 0° C. The resulting solution was stirred for 4 h at RT. The reaction was quenched with sat. NH₄Cl (100 mL) and extracted with 3×100 mL of ethyl acetate. The combined organic layers were dried over Na₂SO₄ and concentrated under vacuum. The crude solid was washed with ethyl acetate/hexane (1:5) (2×100 mL). This resulted in 6.81 g (90%) of the title compound as a yellow solid. MS-ESI: 337.1 (M+1), 335.1 (M−1) in positive and negative ion mode, respectively.

### Step 6: N′-(tert-butyldimethylsilyl)-5-(2-hydroxypropan-2-yl)thiazole-2-sulfonimidamide

Into a 100-mL 3-necked round-bottom flask purged with and maintained under nitrogen was placed a solution of PPh₃Cl₂ (3 g, 9.0 mmol) in CHCl₃ (100 mL). This was followed by the addition of DIEA (1.54 g, 11.9 mmol) dropwise with stirring at RT. The resulting solution was stirred for 10 min at RT. This was followed by the addition of a solution of N-(tert-butyldimethylsilyl)-5-(2-hydroxypropan-2-yl)thiazole-2-sulfonamide (2.0 g, 5.9 mmol) in CHCl₃ (30 mL) dropwise with stirring in an ice/water bath. The resulting solution was stirred for 30 min in an ice/water bath. To the above was introduced NH₃ (g) below 0° C. for 15 minutes. The resulting solution was stirred for 20 minutes at RT. The solids were filtered out and the filtrate was concentrated and the residue was dissolved in 300 mL of ethyl acetate. The solution was washed with brine (2×100 mL), dried over Na₂SO₄, and concentrated under vacuum. The crude solid was washed with CHCl₃ (100 mL). Then the filtrate was concentrated under vacuum, and the residue was further purified by a silica gel column with ethyl acetate/petroleum ether (1:10 to 1:3). The original washed solid and solid from silica gel purification were combined. This resulted in 1.2 g (60%) of the title compound as a white solid. MS-ESI: 336.1 (M+1). ¹H-NMR (300 MHz, DMSO-d₆) δ 7.66 (s, 1H), 7.12 (s, 2H), 5.78 (s, 1H), 1.51 (s, 6H), 0.86 (s, 9H), 0.02 (s, 3H), 0.01 (s, 3H).

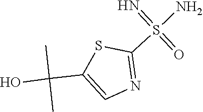

### 5-(2-Hydroxypropan-2-yl)thiazole-2-sulfonimidamide

### Step 7: 5-(2-Hydroxypropan-2-yl)thiazole-2-sulfonimidamide

Into a 50-mL round-bottom flask was placed a solution of N′-(tert-butyldimethylsilyl)-5-(2-hydroxypropan-2-yl)thiazole-2-sulfonimidamide (200 mg, 0.60 mmol), DCM (3 mL), and TFA (0.3 mL). The resulting solution was stirred for 30 min at RT and then was concentrated under vacuum. The crude product was purified by Prep-HPLC using Method E with the following conditions: Column, C18 silica gel, mobile phase, Water (10 mmol/L NH₄HCO₃) and ACN (10% to 50% in 20 min), Detector, UV detection 254/210 nm. This resulted in 100 mg (76%) of the title compound as a light yellow solid. MS-ESI: 222.0 (M+1).

TABLE 2

| | | | Exact |
|---|---|---|---|
| Intermediate | | | Mass |
| # | Structure | IUPAC Name | [M + H]⁺ |
| Intermediate 3 | 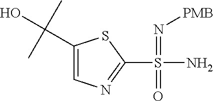 | 5-(2-hydroxypropan-2-y1)-N′-(4- methoxybenzyl)thiazole-2-sulfonimidamide | 342.1 |
| Intermediate 4 | 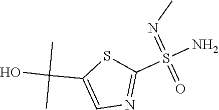 | 5-(2-hydroxypropan-2-y1)-N′- methylthiazole-2-sulfonimidamide | 236.0 |

The Intermediates in the following Table were prepared using similar procedures for converting compound 6 to Intermediate 2 shown in Scheme 7 by substituting ammonia with appropriated amine in Step 6.

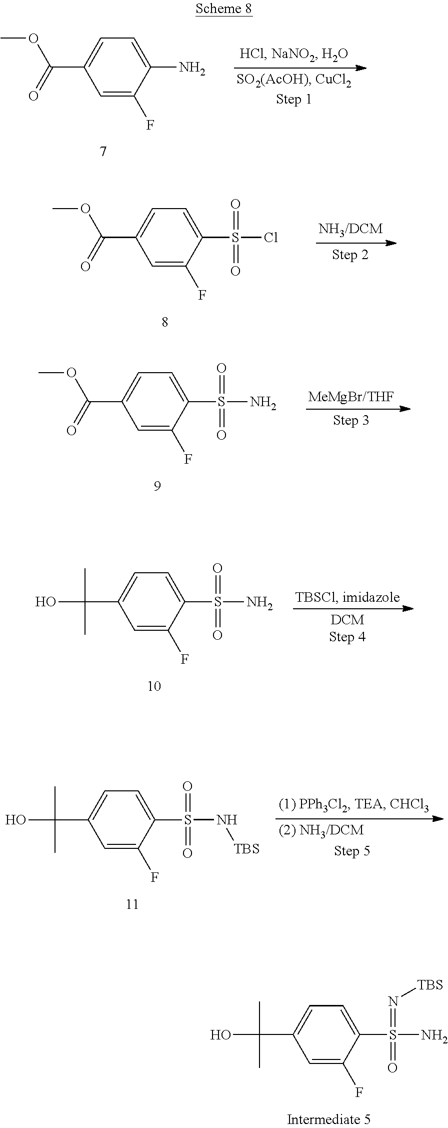

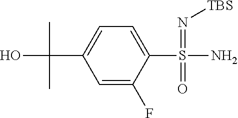

### N′-(tert-butyldimethylsilyl)-2-fluoro-4-(2-hydroxypropan-2-yl)benzenesulfonimidamide

### Step 1: Methyl 4-(chlorosulfonyl)-3-fluorobenzoate

Into a 100-mL round-bottom flask was placed a solution of methyl 4-amino-3-fluorobenzoate (1.0 g, 5.91 mmol) in aq. HCl (6 N, 20 mL). This was followed by the addition of a solution of NaNO₂ (612.4 mg, 8.88 mmol) in water (2 mL) dropwise with stirring at 0° C. The resulting solution was stirred for 30 min at 0° C. The above mixture was added to a saturated solution of SO₂ in AcOH (20 mL) dropwise with stirring at 0° C. Then to the above was added CuCl₂ (0.799 g, 5.96 mmol). The resulting solution was stirred for 1 h at RT and then was quenched by the addition of 20 mL of water. The resulting solution was extracted with 3×20 mL of DCM. The organic layers were combined, dried over anhydrous Na₂SO₄, and then concentrated under vacuum. This resulted in 1 g (crude, 67%) of the title compound as yellow oil. The crude product was used in the next step.

Steps 2-5 used similar procedures for converting compound 3 to Intermediate 1 shown in Scheme 7 to afford Intermediate 5. MS-ESI. 347.2 (M+1).

TABLE 3

| | | | Exact |
|---|---|---|---|
| Intermediate | | | Mass |
| # | Structure | IUPAC Name | [M + H]⁺ |
| Intermediate 6 | 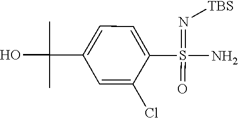 | N′-(tert-butyldimethylsily1)-2-chloro-4-(2-hydroxypropan- 2-yl)benzenesulfonimidamide | 363.1 |
| Intermediate 7 | 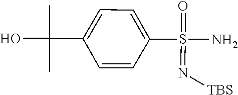 | N′-(tert-butyldimethylsily1)-4-(2-hydroxypropan-2- yl)benzenesulfonimidamide | 329.1 |

The Intermediates in the following Table were prepared using similar procedures for converting compound 7 to Intermediate 5 shown in Scheme 8 from appropriate starting materials.

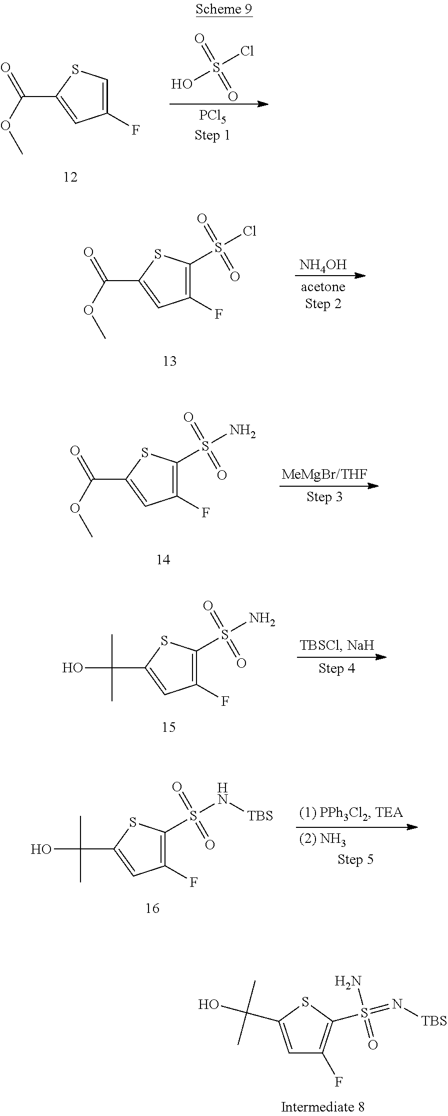

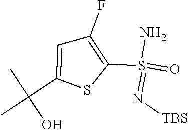

### N′-(tert-butyldimethylsilyl)-3-fluoro-5-(2-hydroxypropan-2-yl)thiophene-2-sulfonimidamide

### Step 1: Methyl 5-(chlorosulfonyl)-4-fluorothiophene-2-carboxylate

Into a 50-mL round-bottom flask was placed a solution of methyl 4-fluorothiophene-2-carboxylate (1.0 g, 6.24 mmol) in CHCl₃ (10 mL). Then to the above was added ClSO₃H (2.18 g, 18.7 mmol). The resulting solution was stirred for 12 h at RT. Then to the above was added PCl₅ (6.5 g, 31.2 mmol). The resulting solution was stirred for 2 h at 50° C. and then was quenched by the addition of 30 mL of water. The resulting solution was extracted with 3×30 mL of ethyl acetate; the organic layers were combined, dried over anhydrous Na₂SO₄, and then concentrated under vacuum. This resulted in 1.2 g (crude, 74%) of the title compound as dark red oil. The crude product was used in the next step.

### Step 2: Methyl 4-fluoro-5-sulfamoylthiophene-2-carboxylate

Into a 50-mL round-bottom flask was placed a solution of methyl 5-(chlorosulfonyl)-4-fluorothiophene-2-carboxylate (600 mg, 2.32 mmol) in acetone (6 mL). Then to the above was added aq. NH₄OH (25% wt., 2 mL). The mixture was stirred for 1 h at RT and then diluted with 10 mL of water. The resulting solution was extracted with 3×10 mL of ethyl acetate; the organic layers were combined, dried over anhydrous Na₂SO₄, and then concentrated under vacuum. This resulted in 500 mg (crude, 90%) of the title compound as yellow oil. MS-ESI: 238.0 (M−1).

Step 3-5 used similar procedures for converting compound 4 to Intermediate 1 shown in Scheme 7 to afford Intermediate 8. MS-ESI: 353.1 (M+1).

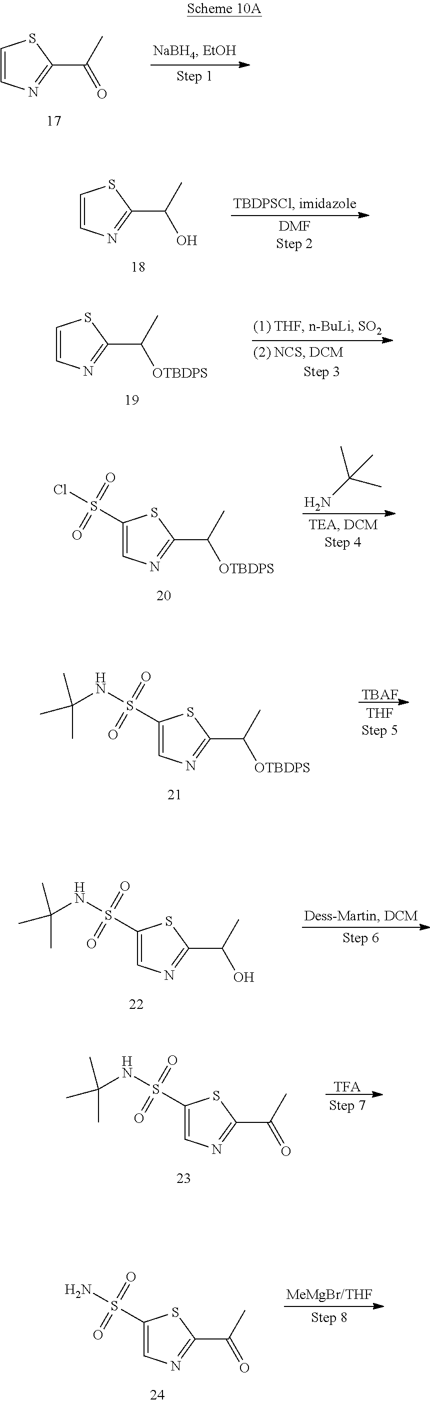

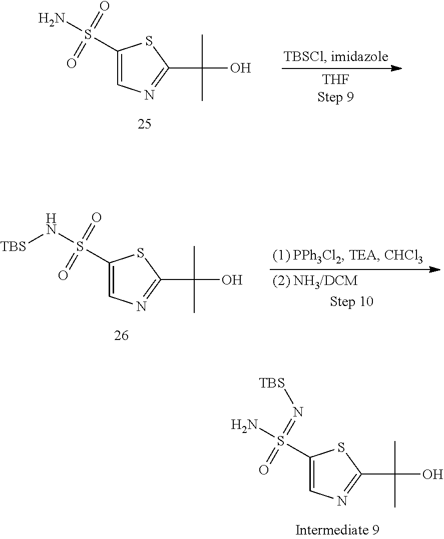

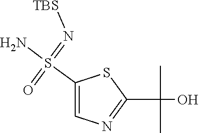

### N′-(tert-butyldimethylsilyl)-2-(2-hydroxypropan-2-yl)thiazole-5-sulfonimidamide

### Step 1: 1-(Thiazol-2-yl)ethanol

Into a 500-mL round-bottom flask was placed 1-(thiazol-2-yl)ethanone (20 g, 157 mmol) in EtOH (200 mL). This was followed by the addition of NaBH₄ (3 g, 81.3 mmol) in portions at 0° C. The resulting solution was stirred for 2 h at RT and was then quenched by the addition of 10 mL of NH₄Cl (sat.). The resulting solution was diluted with 200 mL of water and extracted with 2×200 mL of DCM. The organic layers were combined, dried over anhydrous Na₂SO₄, and then concentrated under vacuum. This resulted in 20 g (98%) of the title compound as light yellow oil. MS-ESI: 130.0 (M+1).

### Step 2: 2-(1-(Tert-butyldiphenylsilyloxy)ethyl)thiazole

Into a 500-mL round-bottom flask was placed 1-(thiazol-2-yl)ethanol (20 g, 154.8 mmol), DMF (150 mL), and imidazole (20.5 g, 301 mmol). This was followed by the addition of TBDPSCl (46 g, 167 mmol) dropwise with stirring at 0° C. The mixture was stirred for 2 h at RT and then was diluted with 300 mL of water. The resulting solution was extracted with 3×200 mL of DCM. The organic layers were combined and concentrated under vacuum. The residue was applied onto a silica gel column and eluted with ethyl acetate/petroleum ether (1:100 to 1:80). This resulted in 55 g (97%) of the title compound as a colorless oil. MS-ESI: 368.1 (M+1).

### Step 3: 2-(1-(Tert-butyldiphenylsilyloxy)ethyl)thiazole-5-sulfonyl chloride

Into a 500-mL 3-necked round-bottom flask purged with and maintained under nitrogen was placed 2-(1-(tert-butyldiphenylsilyloxy)ethyl)thiazole (30 g, 81.6 mmol) and THF (200 mL). This was followed by the addition of n-BuLi/THF (2.5 M, 35.2 mL) dropwise with stirring at −78° C. The resulting solution was stirred for 0.5 h at −78° C., and then SO₂ was introduced into the above reaction mixture. The reaction was slowly warmed to RT, and then NCS (12.8 g, 95.86 mmol) was added. The resulting solution was stirred for 1 h at RT. The solids were filtered out. The resulting filtrate was concentrated under vacuum. This resulted in 30 g (crude, 79%) of the title compound as brown oil. The crude product was used in the next step directly.

### Step 4: N-tert-butyl-2-(1-(tert-butyldiphenylsilyloxy)ethyl)thiazole-5-sulfonamide

Into a 500-mL round-bottom flask was placed 2-(1-(tert-butyldiphenylsilyloxy)ethyl)thiazole-5-sulfonyl chloride (crude, 30 g, 64.4 mmol), DCM (200 mL), and TEA (13 g, 128 mmol). This was followed by the addition of 2-methylpropan-2-amine (5.6 g, 76.6 mmol) dropwise with stirring at 0° C. The resulting solution was stirred for 2 h at RT and then was concentrated under vacuum. The residue was applied onto a silica gel column and eluted with ethyl acetate/petroleum ether (1:30 to 1:20). This resulted in 25 g (77%) of the title compound as brown oil. MS-ESI: 503.2 (M+1).

### Step 5: N-tert-butyl-2-(1-hydroxyethyl)thiazole-5-sulfonamide

Into a 500-mL round-bottom flask was placed N-tert-butyl-2-(1-(tert-butyldiphenylsilyloxy)ethyl)thiazole-5-sulfonamide (25 g, 49.7 mmol), THF (200 mL), and TBAF (30 g, 99.67 mmol). The resulting solution was stirred for 2 h at RT and then was diluted with 200 mL of water. The resulting solution was extracted with 3×200 mL of DCM. The organic layers were combined and concentrated under vacuum. The residue was applied onto a silica gel column and eluted with ethyl acetate/petroleum ether (1:20 to 1:10). This resulted in 12 g (91%) of the title compound as light yellow oil. MS-ESI: 265.1 (M+1).

### Step 6: 2-Acetyl-N-tert-butylthiazole-5-sulfonamide

Into a 500-mL round-bottom flask was placed a solution of N-tert-butyl-2-(1-hydroxyethyl)thiazole-5-sulfonamide (12 g, 45.4 mmol) in DCM (200 mL). To this solution was added Dess-Martin reagent (20 g, 47.2 mmol) in portions at RT. The resulting solution was stirred for 2 h at RT and then was concentrated under vacuum. The residue was applied onto a silica gel column and eluted with ethyl acetate/petroleum ether (1:20 to 1:10). This resulted in 9 g (76%) of the title compound as a light yellow solid. MS-ESI: 263.0 (M+1).

### Step 7: 2-Acetylthiazole-5-sulfonamide

Into a 100-mL round-bottom flask was placed a solution of 2-acetyl-N-tert-butylthiazole-5-sulfonamide (7 g, 26.7 mmol) in DCM (20 mL). To the solution was added TFA (20 mL) at RT. The resulting solution was stirred for 14 h at 70° C. and then was concentrated under vacuum. The residue was applied onto a silica gel column and eluted with ethyl acetate/petroleum ether (1:5 to 1:3). This resulted in 5 g (91%) of the title compound as a yellow solid. MS-ESI: 207.0 (M+1).

### Step 8: 2-(2-Hydroxypropan-2-yl)thiazole-5-sulfonamide

Into a 500-mL 3-necked round-bottom flask purged with and maintained under nitrogen was placed 2-acetylthiazole-5-sulfonamide (5 g, 4.85 mmol) in THF (100 mL). This was followed by the addition of MeMgBr (3 M in THF, 8.1 mL, 24.3 mmol) dropwise with stirring at 0° C. The resulting solution was stirred for 14 h at RT and then was quenched by the addition of 100 mL of NH₄Cl (sat.). The resulting solution was extracted with 2×150 mL of DCM. The organic layers were combined and concentrated under vacuum. The residue was applied onto a silica gel column and eluted with ethyl acetate/petroleum ether (1:5 to 1:3). This resulted in 2.9 g (54%) of the title compound as a light yellow solid. MS-ESI: 223.0 (M+1).

### Step 9: N-(tert-butyldimethylsilyl)-2-(2-hydroxypropan-2-yl)thiazole-5-sulfonamide

Into a 100-mL 3-necked round-bottom flask purged with and maintained under nitrogen was placed a solution of 2-(2-hydroxypropan-2-yl)thiazole-5-sulfonamide (1.5 g, 6.75 mmol) in THF (20 mL). Then to the above was added imidazole (0.92 g, 13.5 mmol). This was followed by the addition of a solution of TBSCl (5.1 g, 34 mmol) in THF (5 mL) dropwise with stirring over 2 min at 0° C. The resulting solution was stirred for 16 h at RT and then was concentrated under vacuum. The residue was applied onto a silica gel column and eluted with ethyl acetate/petroleum ether (1:3 to 1:2). This resulted in 1.13 g (50%) of the title compound as a yellow solid. MS-ESI: 337.1 (M+1).

Steps 10 used similar procedures employed for converting compound 6 to Intermediate 1 shown in Scheme 7 to afford Intermediate 9. MS-ESI: 336.1 (M+1).

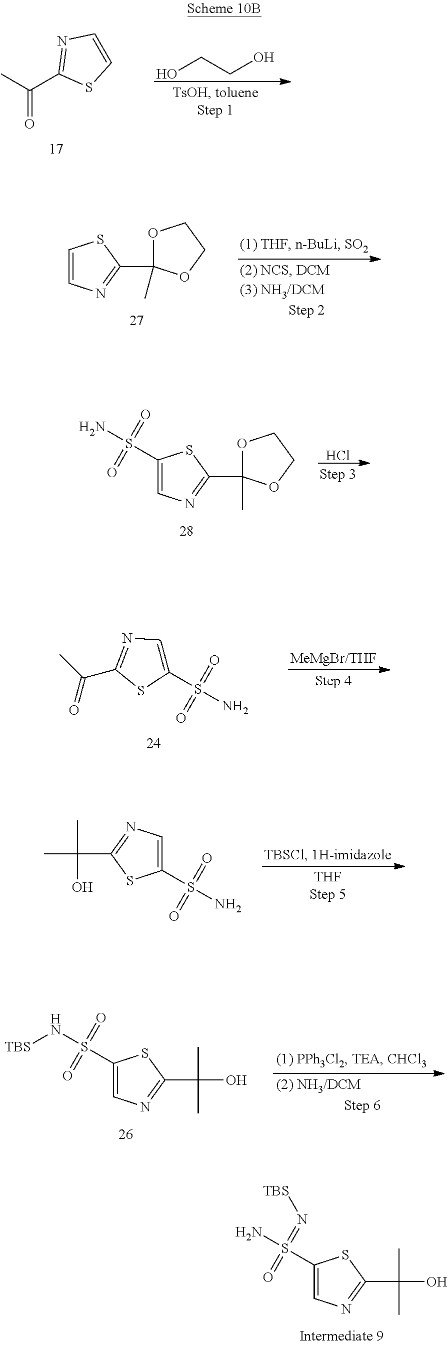

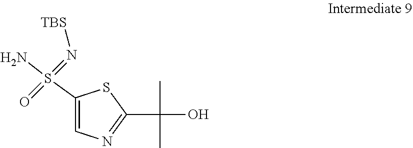

### N′-(tert-butyldimethylsilyl)-2-(2-hydroxypropan-2-yl)thiazole-5-sulfonimidamide

### Step 1: 2-(2-Methyl-1,3-dioxolan-2-yl)thiazole

Into a 500-mL round-bottom flask was placed a solution of 1-(thiazol-2-yl)ethanone (20 g, 157 mmol) in toluene (300 mL). To the solution was added TsOH (2.7 g, 15.7 mmol)) and ethane-1,2-diol (19.5 g, 314 mmol). The resulting solution was refluxed overnight, and water was separated from the solution during refluxing. The resulting solution was diluted with 200 mL of water and extracted with 2×100 mL of ethyl acetate. The organic layers were combined and dried over anhydrous Na₂SO₄, then concentrated under vacuum. This resulted in 26.6 g (99%) of the title compound as light yellow oil. MS-ESI: 172.0 (M+1).

### Step 2: 2-(2-Methyl-1,3-dioxolan-2-yl)thiazole-5-sulfonamide

Into a 500-mL 3-necked round-bottom flask purged with and maintained under nitrogen was placed a solution of 2-(2-methyl-1,3-dioxolan-2-yl)thiazole (14 g, 81.6 mmol) in THF (200 mL). This was followed by the addition of n-BuLi (2.5 M in THF, 35.2 mL) dropwise with stirring at −78° C. The resulting solution was stirred for 0.5 h at −78° C. and then SO₂ gas was introduced into the above reaction mixture. The reaction was slowly warmed to RT and then NCS (12.8 g, 95.86 mmol) was added. The resulting solution was stirred for 1 h at RT. The solids were filtered out. The resulting filtrate was concentrated under vacuum and was then diluted with DCM (160 mL). To the above was added a saturated solution of ammonia in DCM (300 mL). The resulting solution was stirred for 3 h at RT and then was concentrated under vacuum. The residue was applied onto a silica gel column and eluted with a gradient of ethyl acetate/petroleum ether (1:20 to 1:5). This resulted in 12.5 g (61%) of the title compound as a yellow solid. MS-ESI: 251.0 (M+1).

### Step 3: 2-Acetylthiazole-5-sulfonamide

Into a 250-mL round-bottom flask was placed a solution of 2-(2-methyl-1,3-dioxolan-2-yl)thiazole-5-sulfonamide (12.5 g, 50 mmol) in THF (125 mL). To the above was added aq. HCl (4 N, 50 mL). The resulting solution was stirred for 6 h at 70° C. The resulting solution was diluted with 100 mL of water and extracted with 2×200 mL of ethyl acetate. The organic layers were combined, dried over anhydrous Na₂SO₄, and then concentrated under vacuum. The residue was applied onto a silica gel column and eluted with a gradient of ethyl acetate/petroleum ether (1:2 to 1:1). This resulted in 9.3 g (90%) of the title compound as a yellow solid. MS-ESI: 207.0 (M+1). Steps 4-6 used the same procedures for converting compound 24 to Intermediate 9 shown in Scheme 10A to afford Intermediate 9. MS-ESI: 336.1 (M+1).

TABLE 4

| | | | Exact |
|---|---|---|---|
| Intermediate | | | Mass |
| # | Structure | IUPAC Name | [M + H]⁺ |
| Intermediate 10 | 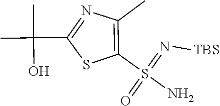 | N′-(tert-butyldimethylsily1)-2-(2-hydroxypropan-2- y1)-4-methylthiazole-5-sulfonimidamide | 350.2 |

The Intermediates in the following Table were prepared using the similar procedures for converting compound 17 to Intermediate 9 shown in Scheme 10B from appropriate starting materials.

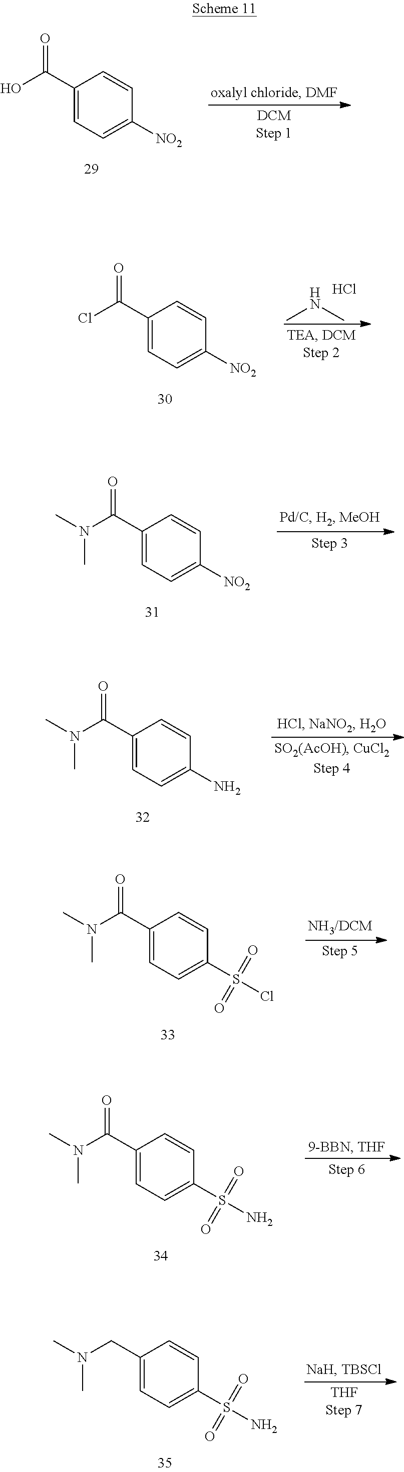

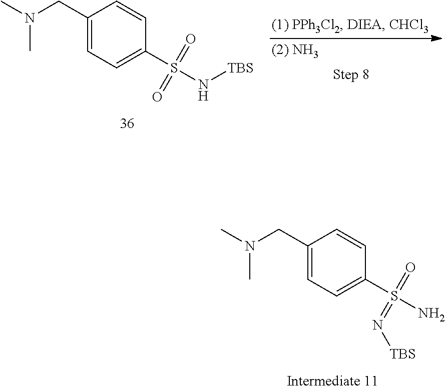

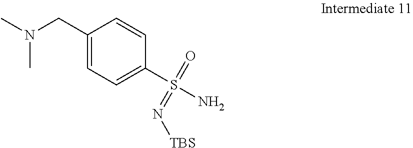

### N′-(tert-butyldimethylsilyl)-4-((dimethylamino)methyl)benzenesulfonimidamide

### Step 1: 4-Nitrobenzoyl Chloride

Into a 500-mL round-bottom flask was placed 4-nitrobenzoic acid (20 g, 120 mmol), DCM (200 mL), and DMF (0.2 mL). This was followed by the addition of oxalyl chloride (15 mL, 135 mmol) dropwise with stirring at 0° C. The resulting solution was stirred for 4 h at RT and then was concentrated under vacuum. This resulted in 22 g (crude) of the title compound as yellow oil. The crude product was used in the next step.

### Step 2: N,N-dimethyl-4-nitrobenzamide

Into a 500-mL round-bottom flask was placed dimethylamine hydrochloride (9.8 g, 120 mmol), DCM (200 mL), and TEA (41.5 mL, 300 mmol). This was followed by the addition of 4-nitrobenzoyl chloride (22 g, crude) dropwise with stirring at 0° C. The resulting solution was stirred for 6 h at RT and then was concentrated under vacuum. The resulting mixture was washed with 2×50 mL of water. The solids were collected by filtration. This resulted in 16 g (69%, 2 steps) of the title compound as a white solid. MS-ESI: 195.1 (M+1).

### Step 3: 4-Amino-N,N-dimethylbenzamide

Into a 250-mL round-bottom flask was placed N,N-dimethyl-4-nitrobenzamide (16 g, 82.4 mmol), MeOH (100 mL), and Pd/C (10% wt., 1 g). The flask was evacuated and flushed three times with hydrogen. The resulting solution was stirred for 12 h at RT under an atmosphere of hydrogen. The Pd/C catalysts were filtered out, and the filtrate was concentrated under vacuum. This resulted in 13 g (96%) of the title compound as a white solid. MS-ESI: 165.1 (M+1).

### Step 4: 4-(Dimethylcarbamoyl)benzene-1-sulfonyl chloride

Into a 50-mL round-bottom flask was placed 4-amino-N,N-dimethylbenzamide (3 g, 18.3 mmol) and HCl (6 M, 12 mL). This was followed by the addition of a solution of NaNO₂ (1.5 g, 21.7 mmol) in water (3 mL) dropwise with stirring at 0° C. The resulting solution was stirred for 30 min at 0° C. The above mixture was added to a saturated solution of SO₂ in AcOH (100 mL) dropwise with stirring at 0° C. To the above was added CuCl₂ (4.8 g, 35.7 mmol). The resulting solution was stirred for 2 h at RT and then was quenched by the addition of 100 mL of water. The resulting solution was extracted with 2×100 mL of DCM. The organic layers were combined, dried over anhydrous Na₂SO₄, and then concentrated under vacuum. This resulted in 5 g (crude) of the title compound as yellow oil. The crude product was used in the next step.

### Step 5: N,N-dimethyl-4-sulfamoylbenzamide

Into a 250-mL round-bottom flask was placed 4-(dimethylcarbamoyl)benzene-1-sulfonyl chloride (5 g, 20.2 mmol) in DCM (20 mL). To the above was added a saturated solution of ammonia in DCM (80 mL). The resulting solution was stirred for 2 h at RT and then was concentrated under vacuum. The resulting mixture was washed with 3×100 mL of ethyl acetate. The solids were filtered out. The resulting filtrate was concentrated under vacuum. This resulted in 3.1 g (67%) of the title compound as a white solid. MS-ESI: 229.1 (M+1).

### Step 6: 4-((Dimethylamino)methyl)benzenesulfonamide

Into a 100-mL round-bottom flask purged with and maintained under nitrogen was placed a solution of N,N-dimethyl-4-sulfamoylbenzamide (1.8 g, 7.9 mmol) in THF (50 mL). This was followed by the addition of 9-BBN (5.8 g, 47.5 mmol) in portions at 0° C. The resulting solution was stirred for 12 h at 70° C. and then was quenched by the addition of 20 mL of water/ice. The resulting solution was extracted with 3×100 mL of ethyl acetate and the organic layers were combined. The resulting mixture was washed with 200 mL of water and then the organic layer was concentrated under vacuum. The residue was applied onto a silica gel column and eluted with DCM/MeOH (20:1 to 15:1). This resulted in 1 g (59%) of the title compound as a white solid. MS-ESI: 215.1 (M+1).

### Step 7: N-(tert-butyldimethylsilyl)-4-((dimethylamino)methyl)benzenesulfonamide

Into a 250-mL round-bottom flask was placed a solution of 4-((dimethylamino)methyl)benzenesulfonamide (500 mg, 2.33 mmol) in THF (40 mL). This was followed by the addition NaH (60% wt., 170 mg) in portions at 0° C. Then TBSCl (1.75 g, 11.6 mmol) was added. The resulting solution was stirred for 2 h at RT and then was quenched by the addition of 30 mL of water. The resulting solution was extracted with 3×40 mL of ethyl acetate and the organic layers combined and concentrated under vacuum. The residue was applied onto a silica gel column and eluted with DCM/MeOH (30:1 to 20:1). This resulted in 540 mg (70%) of the title compound as a yellow solid. MS-ESI: 329.2 (M+1).

Step 8 used similar procedures for converting compound 6 to Intermediate 1 shown in Scheme 7 to afford Intermediate 11. MS-ESI: 328.2 (M+1).

TABLE 4

| | | | Exact |
|---|---|---|---|
| Intermediate | | | Mass |
| # | Structure | IUPAC Name | [M + H]⁺ |
| Intermediate 12 | 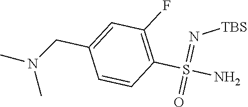 | N′-(tert-butyldimethylsily1)-4-((dimethylamino)methyl)- 2-fluorobenzenesulfonimidamide | 346.2 |

The Intermediates in the following Table were prepared using the similar procedures for converting compound 29 to Intermediate 11 shown in Scheme 11 from appropriate starting materials.

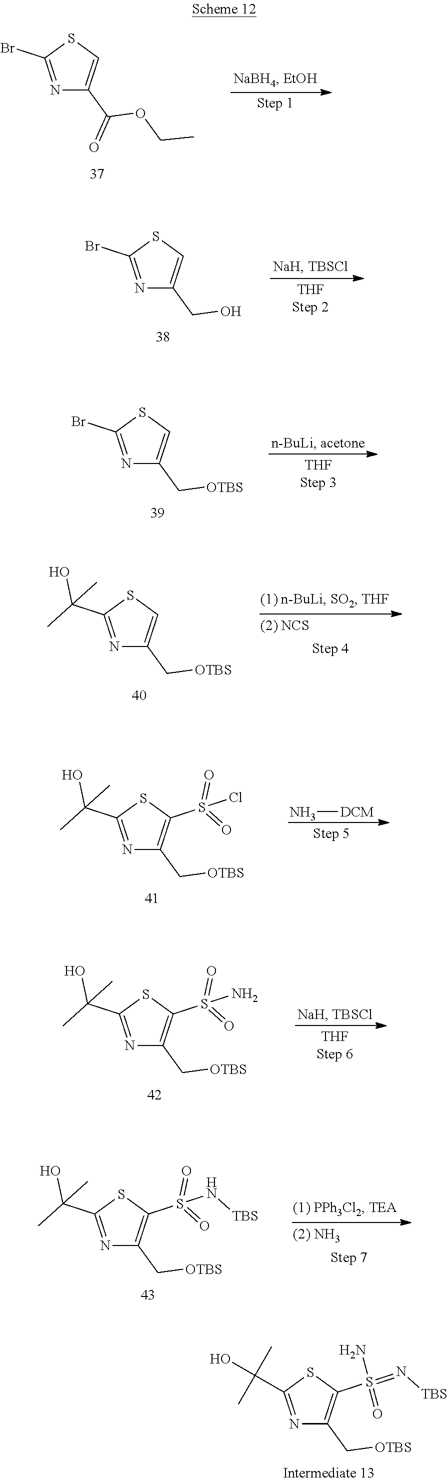

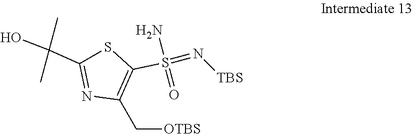

### N′-(tert-butyldimethylsilyl)-4-((tert-butyldimethylsilyloxy)methyl)-2-(2-hydroxypropan-2-yl)thiazole-5-sulfonimidamide

### Step 1: (2-Bromothiazol-4-yl)methanol

Into a 100-mL round-bottom flask was placed a solution of ethyl 2-bromo-1,3-thiazole-4-carboxylate (3 g, 12.71 mmol) in EtOH (30 mL). NaBH₄ (1.0 g, 25.41 mmol) was added in portions with an ice/water bath. The resulting solution was stirred for 3 hr at room temperature. The reaction was then quenched by the addition of 100 mL of water in an ice/water bath. The resulting solution was extracted with 3×100 ml of ethyl acetate, and the combined organic layers were concentrated. This resulted in 2 g (81%) of the title compound as yellow oil. MS-ESI. 196.2, 194.2 (M+1).

### Step 2: 2-Bromo-4-((tert-butyldimethylsilyloxy)methyl)thiazole

Into a 100-mL round-bottom flask was placed a solution of (2-bromo-1,3-thiazol-4-yl)methanol (2.0 g, 10.31 mmol) in THF (20 mL). To the solution was added NaH (60% wt., 1.2 g, 30.92 mmol) in portions with an ice/water bath. After stirring for 15 minutes at RT, a solution of TBSCl (4.7 g, 30.92 mmol) in THF (5 mL) was added dropwise in an ice/water bath. The resulting solution was stirred for 2 hr at RT. The reaction was then quenched by the addition of 50 mL of water. The resulting solution was extracted with 3×100 ml of ethyl acetate, the organic layers were combined, dried over anhydrous Na₂SO₄ and concentrated under vacuum. The residue was applied onto a silica gel column and eluted with ethyl acetate/petroleum ether (1:30). This resulted in 2.5 g (79%) of the title compound as yellow oil. MS-ESI: 310.2, 308.2 (M+1).

### Step 3: 2-(4-((Tert-butyldimethylsilyloxy)methyl)thiazol-2-yl)propan-2-ol

Into a 100-mL round-bottom flask purged and maintained with an inert atmosphere of nitrogen was placed a solution of 2-bromo-4-[[(tert-butyldimethylsilyl)oxy]methyl]-1,3-thiazole (2.5 g, 8.11 mmol) in THF (30 mL). To this solution was added n-BuLi (2.5 M in hexane, 4.86 mL, 12.16 mmol) dropwise at −78° C.; and the resulting mixture was stirred for 30 min at −78° C. To the above was added acetone (0.9 g, 16.22 mmol) dropwise at −78° C. The ensuing solution was then stirred for 1 hr at RT, after which the reaction was quenched by the addition of 100 mL of water. The resulting solution was extracted with 3×100 ml of ethyl acetate; the organic layers were combined, dried over anhydrous Na₂SO₄, and concentrated under vacuum. The residue was applied onto a silica gel column and eluted with ethyl acetate/petroleum ether (1:10). This resulted in 2 g (86%) of the title compound as yellow oil. MS-ESI: 288.2 (M+1).

### Step 4: 4-((Tert-butyldimethylsilyloxy)methyl)-2-(2-hydroxypropan-2-yl)thiazole-5-sulfonyl Chloride

Into a 250-mL round-bottom flask purged and maintained with an inert atmosphere of nitrogen was placed a solution of 2-(4-[[(tert-butyldimethylsilyl)oxy]methyl]-1,3-thiazol-2-yl)propan-2-ol (2 g, 6.96 mmol) in THF (20 mL). To this solution was added n-BuLi (2.5 M in hexane, 8.4 mL, 20.9 mmol) dropwise at −78° C.; the mixture was stirred for 30 min at −78° C. Then SO₂ was introduced in this solution for 10 minutes below −30° C. and stirred for 30 min at RT. The resulting solution was concentrated under vacuum. The crude solid was dissolved in DCM (30 ml), followed by the addition of NCS (1.4 g, 10.4 mmol) in portions in an ice/water bath. The solution was stirred for 2 hr at RT. The resulting mixture was concentrated under vacuum. This resulted in 2.5 g (crude) of the title compound as a yellow solid.

### Step 5: 4-((Tert-butyldimethylsilyloxy)methyl)-2-(2-hydroxypropan-2-yl)thiazole-5-sulfonamide

Into a 100-mL round-bottom flask was placed a solution of 4-[[(tert-butyldimethylsilyl)oxy]methyl]-2-(2-hydroxypropan-2-yl)-1,3-thiazole-5-sulfonyl chloride (2.5 g, 6.48 mmol) in DCM (30 mL). To the above was added a saturated solution of ammonia in DCM (10 mL) in an ice/water bath. The resulting solution was stirred for 1 hr at room temperature. The resulting mixture was concentrated. The residue was applied onto a silica gel column and eluted with ethyl acetate/petroleum ether (1:5). This resulted in 1.2 g (51%) of the title compound as yellow oil. MS-ESI: 367.2 (M+1).

### Step 6: N-(tert-butyldimethylsilyl)-4-((tert-butyldimethylsilyloxy)methyl)-2-(2-hydroxypropan-2-yl)thiazole-5-sulfonamide

To a solution of 2-(2-hydroxypropan-2-yl)-1,3-thiazole-5-sulfonamide (1.2 g, 3.27 mmol) in THF (20 mL), NaH (60% wt., 0.4 g, 9.82 mmol) was added in portions with an ice/water bath. After stirring for 15 minutes at RT, a solution of TBSCl (1.5 g, 9.82 mmol) in THF (5 mL) was added dropwise in an ice/water bath. The resulting solution was stirred for 2 hr at RT. The reaction was quenched by the addition of 100 mL of water. The resulting solution was extracted with 3×100 ml of ethyl acetate, the organic layers were combined, dried over anhydrous Na₂SO₄ and concentrated under vacuum. This resulted in 1.3 g (83%) of the title compound as yellow oil. MS-ESI: 481.2 (M+1).

### Step 7: N′-(tert-butyldimethylsilyl)-4-((tert-butyldimethylsilyloxy)methyl)-2-(2-hydroxypropan-2-yl)thiazole-5-sulfonimidamide

Into a 100-mL round-bottom flask purged and maintained with an inert atmosphere of nitrogen was placed a solution of PPh₃Cl₂ (1.4 g, 4.06 mmol) in CHCl₃ (10 mL), TEA (0.8 g, 8.11 mmol) was added dropwise in an ice/water bath. The solution was stirred at RT for 20 minutes. To this solution was added N-(tert-butyldimethylsilyl)-4-[[(tert-butyldimethylsilyl)oxy]methyl]-2-(2-hydroxypropan-2-yl)-1,3-thiazole-5-sulfonamide (1.3 g, 2.70 mmol) in CHCl₃ (10 mL) dropwise in ice/water bath, the solution was stirred for 0.5 hr at RT. A saturated solution of ammonia in DCM (20 mL) was poured into this solution at 0° C. The solution was stirred for 1 hr at RT. The resulting solution was concentrated under vacuum. The residue was applied onto a silica gel column and eluted with ethyl acetate/petroleum ether (1:5). This resulted in 600 mg (46%) of the title compound as a yellow solid. MS-ESI: 480.2 (M+1).

Schemes for phenylacetic acids Intermediates: Schemes 13-22 illustrate the preparation of phenylacetic acid intermediates.

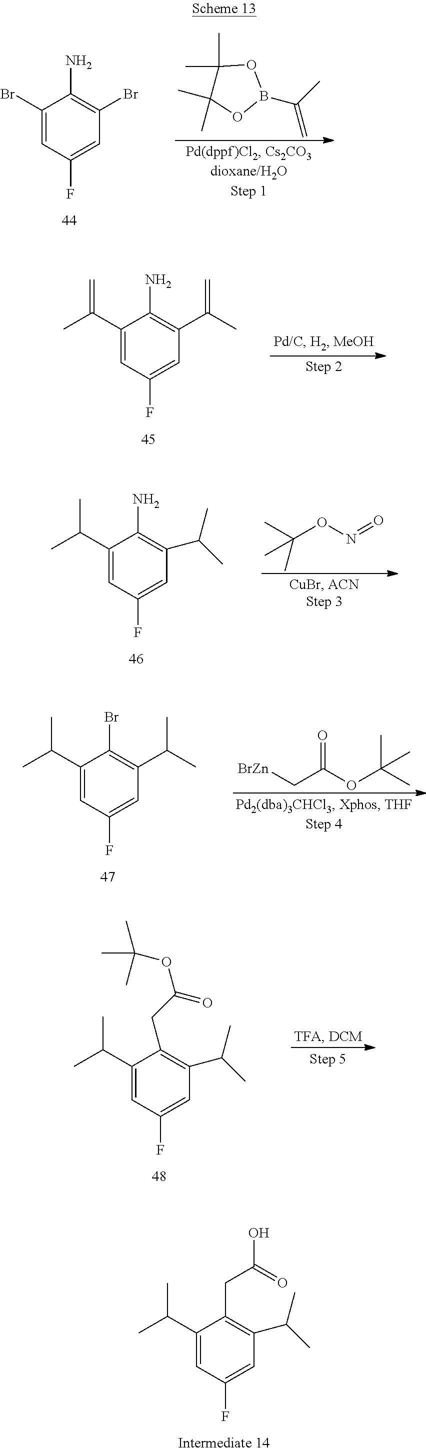

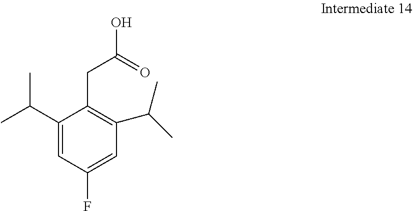 [SMILES: CC(C)c1cc(F)cc(C(C)C)c1CC(=O)O]

### 2-(4-Fluoro-2,6-diisopropylphenyl)acetic Acid

### Step 1: 4-Fluoro-2,6-di(prop-1-en-2-yl)aniline

Into a 500-mL round-bottom flask purged with and maintained under nitrogen was placed 2,6-dibromo-4-fluoroaniline (15 g, 55.8 mmol), dioxane (150 mL), water (15 mL), Cs₂CO₃ (55 g, 169 mmol), 4,4,5,5-tetramethyl-2-(prop-1-en-2-yl)-1,3,2-dioxaborolane (25 g, 149 mmol), and Pd(dppf)Cl₂ (4 g, 5.47 mmol). The resulting solution was stirred for 15 h at 100° C. and then was concentrated under vacuum. The mixture was diluted with 300 mL water, and extracted with ethyl acetate (3×300 mL). The organic layers were combined, dried over anhydrous Na₂SO₄, and concentrated under vacuum. The residue was applied onto a silica gel column and eluted with ethyl acetate/petroleum ether (1:10 to 1:8). This resulted in 9.2 g (86%) of the title compound as brown oil. MS-ESI: 192.1 (M+1).

### Step 2: 4-Fluoro-2,6-bis(propan-2-yl)aniline

Into a 500-mL round-bottom flask was placed 4-fluoro-2,6-bis(prop-1-en-2-yl)aniline (9.2 g, 48.1 mmol) in MeOH (200 mL). Then Pd/C (10% wt, 900 mg) was added. The flask was evacuated and flushed three times with hydrogen. The resulting solution was stirred for 12 h at RT under an atmosphere of hydrogen. The solids were filtered out. The resulting mixture was concentrated under vacuum. The residue was applied onto a silica gel column and eluted with ethyl acetate/petroleum ether (1:10 to 1:8). This resulted in 7.2 g (77%) of the title compound as brown oil. MS-ESI: 196.1 (M+1).

### Step 3: 2-Bromo-5-fluoro-1,3-bis(propan-2-yl)benzene

Into a 500-mL round-bottom flask purged with and maintained under nitrogen was placed 4-fluoro-2,6-bis(propan-2-yl)aniline (7 g, 35.9 mmol), ACN (300 mL), and CuBr (7.71 g, 53.9 mmol). This was followed by the addition of tert-butyl nitrite (5.55 g, 53.8 mmol) dropwise with stirring at 0° C. The resulting solution was stirred for 3 h at 60° C. and then was concentrated under vacuum. The residue was applied onto a silica gel column eluted with petroleum ether. This resulted in 3.0 g (32%) of the title compound as yellow oil. ¹H NMR (400 MHz, DMSO-d₆): δ 7.09 (d, J=9.8 Hz, 2H), 3.40 (hept, J=6.9 Hz, 2H), 1.20 (d, J=6.8 Hz, 12H).

### Step 4: Tert-butyl 2-[4-fluoro-2,6-bis(propan-2-yl)phenyl]acetate

Into a 250-mL 3-necked round-bottom flask purged with and maintained under nitrogen was placed 2-bromo-5-fluoro-1,3-bis(propan-2-yl)benzene (3.0 g, 11.6 mmol), THF (150 mL), X-phos (553 mg, 1.16 mmol), and Pd₂(dba)₃CHCl₃ (600 mg, 0.58 mmol). The resulting solution was stirred for 0.5 h at RT. Then, to the above, tert-butyl 2-(bromozincio)acetate (6.0 g, 23.04 mmol) was added. The resulting solution was stirred for 5 h at 70° C., after which it was quenched by the addition of 100 mL of NH₄Cl (sat.). The resulting solution was extracted with 3×100 mL of ethyl acetate, and the organic layers combined and concentrated under vacuum. The residue was applied onto a silica gel column and eluted with ethyl acetate/petroleum ether (1:100 to 3:97). This resulted in 3.14 g (92%) of the title compound as yellow oil. ¹H NMR (400 MHz, DMSO-d₆) δ 6.93 (d, J=10.4 Hz, 2H), 3.67 (s, 2H), 3.19-3.07 (m, 2H), 1.39 (s, 9H), 1.15 (d, J=6.7 Hz, 12H).

### Step 5: 2-(4-Fluoro-2,6-diisopropylphenyl)acetic Acid

Into a 50-mL round-bottom flask was placed tert-butyl 2-[4-fluoro-2,6-bis(propan-2-yl)phenyl]acetate (1.56 g, 5.30 mmol), DCM (10 mL), and TFA (10 mL). The resulting solution was stirred for 3 h at RT and was then concentrated under vacuum. The crude product was dissolved in 100 mL of NaOH (4 N) and washed with 3×50 mL of DCM to remove impurities. The pH value of aqueous phase was adjusted to 2 with HCl (4 N); the aqueous phase was then extracted with 3×100 mL of DCM. The organic layers were combined, dried over anhydrous Na₂SO₄, and concentrated under vacuum. This resulted in 1.09 g (86%) of the title compound as a light yellow solid. MS-ESI. 237.1 (M−1).

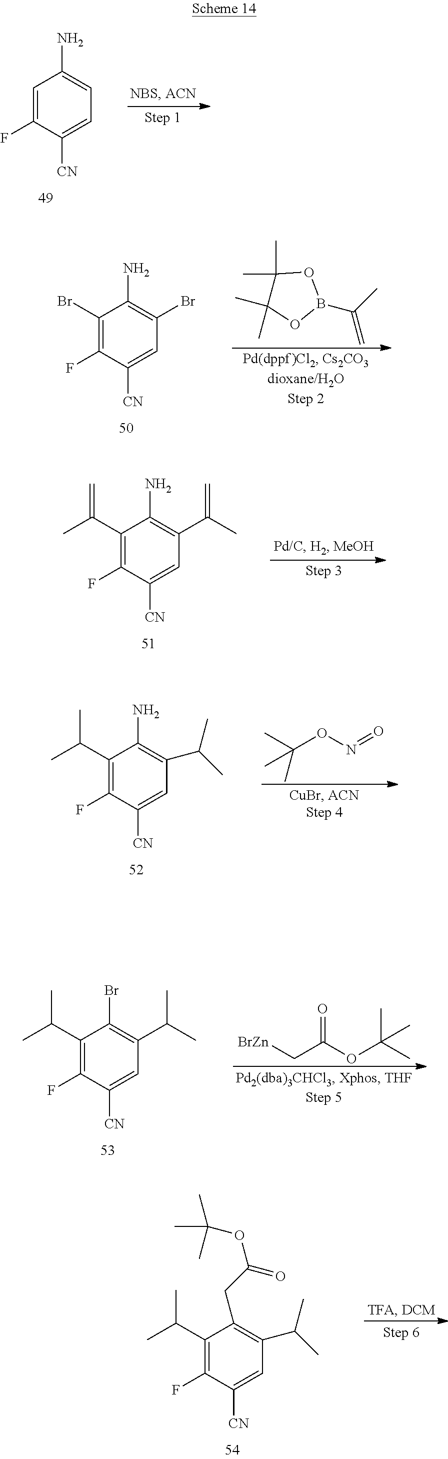

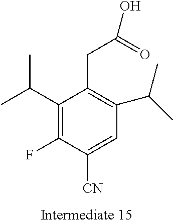 [SMILES: CC(C)c1cc(C#N)c(F)c(C(C)C)c1CC(=O)O]

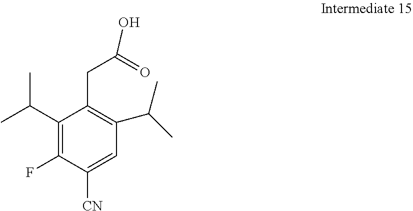 [SMILES: CC(C)c1cc(C#N)c(F)c(C(C)C)c1CC(=O)O]

### 2-(4-Cyano-3-fluoro-2,6-diisopropylphenyl)acetic Acid

### Step 1: 4-amino-3,5-dibromo-2-fluorobenzonitrile

Into a 1000-mL round-bottom flask was placed 4-amino-2-fluorobenzonitrile (25 g, 184 mmol), ACN (500 mL), and NBS (81.7 g, 459 mmol). The resulting solution was stirred overnight at 75° C. and then was concentrated under vacuum. The residue was applied onto a silica gel column and eluted with ethyl acetate/petroleum ether (1:100 to 1:98). This resulted in 50 g (93%) of the title compound as brown oil. MS-ESI: 294.9/292.9/296.9 (M+1).

Steps 2-6 used similar procedures for converting compound 44 to Intermediate 14 shown in Scheme 13 to afford Intermediate 15. MS-ESI: 262.1 (M−1).

TABLE 5
The Intermediate in the following Table was prepared using the similar procedures for
converting compound 49 to Intermediate 15 shown in Scheme 14 from appropriated starting
materials.

| Intermediate | | | Exact |
|---|---|---|---|
| | | | Mass |
| # | Structure | IUPAC Name | [M + H]⁺ |
| Intermediate 16 | 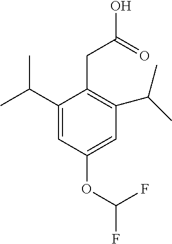 [SMILES: CC(C)c1cc(OC(F)F)cc(C(C)C)c1CC(=O)O] | 2-(4-(Difluoromethoxy)-2,6-diisopropylphenyflacetic acid | 285.1 |

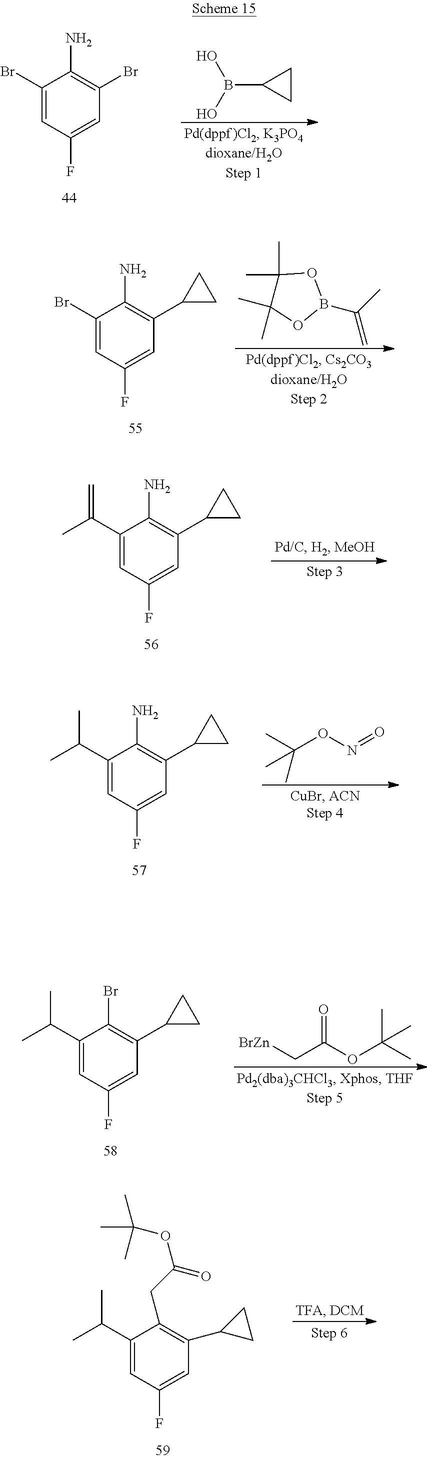

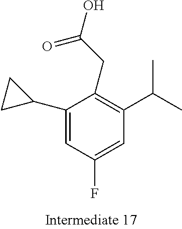 [SMILES: CC(C)c1cc(F)cc(C2CC2)c1CC(=O)O]

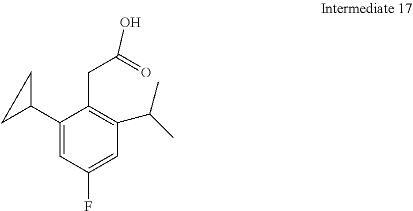 [SMILES: CC(C)c1cc(F)cc(C2CC2)c1CC(=O)O]

### 2-(2-Cyclopropyl-4-fluoro-6-isopropylphenyl)acetic Acid

### Step 1: 2-Bromo-6-cyclopropyl-4-fluorobenzenamine

Into a 500-mL round-bottom flask purged with and maintained under nitrogen was placed 2,6-dibromo-4-fluorobenzenamine (10 g, 37.2 mmol), 1,4-dioxane (200 mL), water (10 mL), K₃PO₄ (23.6 g, 111 mmol), cyclopropylboronic acid (9.59 g, 112 mmol), and Pd(dppf)Cl₂ (1.36 g, 1.86 mmol). The resulting solution was stirred overnight at 90° C. and then was concentrated under vacuum. The residue was applied onto a silica gel column and eluted with ethyl acetate/petroleum ether (1:40 to 1:20). This resulted in 3.4 g (40%) of the title compound as light yellow oil. MS-ESI: 230.0 (M+1).

### Step 2: 2-Cyclopropyl-4-fluoro-6-(prop-1-en-2-yl)benzenamine

Into a 250-mL round-bottom flask purged with and maintained under nitrogen was placed 2-bromo-6-cyclopropyl-4-fluorobenzenamine (3.4 g, 14.8 mmol), dioxane (100 mL), water (10 mL), Cs₂CO₃ (14.5 g, 44.5 mmol), 4,4,5,5-tetramethyl-2-(prop-1-en-2-yl)-1,3,2-dioxaborolane (3.75 g, 22.3 mmol), and Pd(dppf)Cl₂ (1.1 g, 1.50 mmol). The resulting solution was stirred overnight at 110° C. and then concentrated under vacuum. The residue was applied onto a silica gel column and eluted with ethyl acetate/petroleum ether (1:40 to 1:20). This resulted in 1.7 g (60%) of the title compound as light yellow oil. MS-ESI: 192.1 (M+1).

### Step 3: 2-Cyclopropyl-4-fluoro-6-isopropylbenzenamine

Into a 250-mL round-bottom flask was placed 2-cyclopropyl-4-fluoro-6-(prop-1-en-2-yl)benzenamine (1.7 g, 8.89 mmol), and MeOH (100 mL). Then Pd/C (10% wt, 100 mg) was added. The flask was evacuated and flushed three times with hydrogen. The resulting solution was stirred for 3 h at RT under an atmosphere of hydrogen. The solids were filtered out. The resulting mixture was concentrated under vacuum. This resulted in 1.53 g (89%) of the title compound as yellow oil. MS-ESI: 194.1 (M+1).

Steps 4-6 used similar procedures for converting compound 46 to Intermediate 14 shown in Scheme 13 to afford Intermediate 17. MS-ESI: 235.1 (M−1).

TABLE 7

| | | | Exact |
|---|---|---|---|
| Intermediate | | | Mass |
| # | Structure | IUPAC Name | [M + H]⁺ |
| Intermediate 18 | 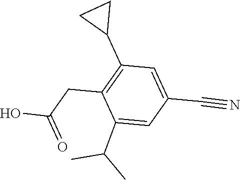 [SMILES: CC(C)c1cc(C#N)cc(C2CC2)c1CC(=O)O] | 2-(4-cyano-2-cyclopropy1-6-isopropylphenyl)acetic acid | 242.1 |
| Intermediate 19 | 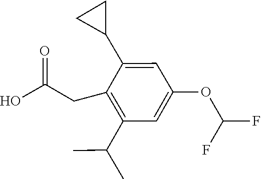 [SMILES: CC(C)c1cc(OC(F)F)cc(C2CC2)c1CC(=O)O] | 2-(2-cyclopropy1-4-(difluoromethoxy)-6- isopropylphenyl)acetic acid | 283.1 |

The Intermediates in the following Table were prepared using the similar procedures for converting compound 44 to Intermediate 17 shown in Scheme 15 from appropriate starting materials.

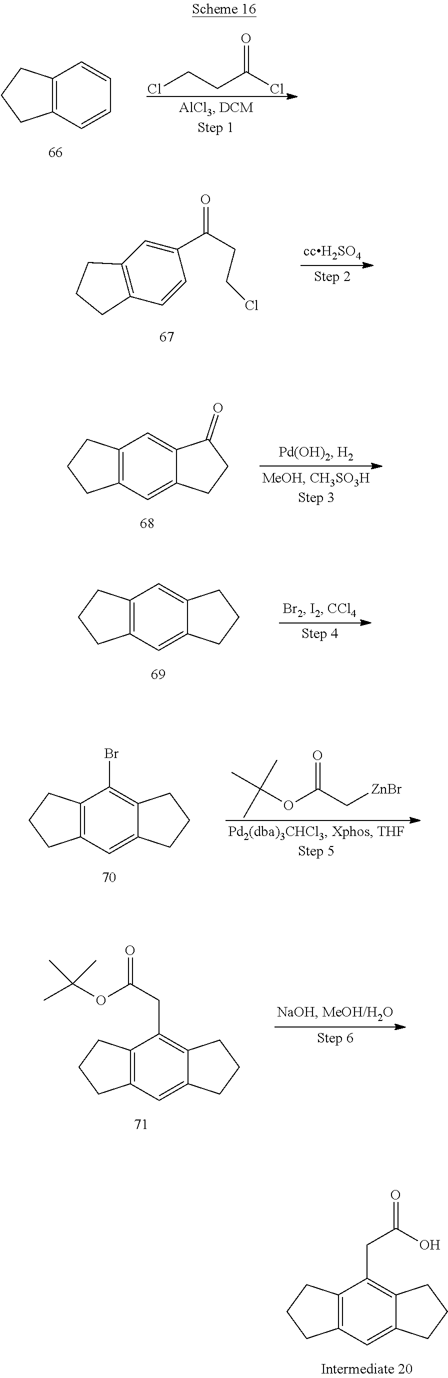

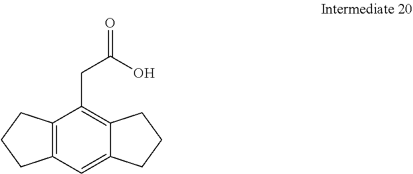 [SMILES: O=C(O)Cc1c2c(cc3c1CCC3)CCC2]

### 2-(1,2,3,5,6,7-Hexahydro-s-indacen-4-yl)acetic Acid

### Step 1: 3-Chloro-1-(2,3-dihydro-1H-inden-5-yl)propan-1-one

Into a 1000-mL round-bottom flask was placed a solution of AlCl₃ (37 g, 278 mmol) in DCM (400 mL). This was followed by the addition of a solution of 2,3-dihydro-1H-indene (30 g, 254 mmol) and 3-chloropropanoyl chloride (32.1 g, 253 mmol) in DCM (100 mL) dropwise with stirring at −10° C. in 30 min. The resulting solution was stirred for 16 h at RT. Then the reaction mixture was added dropwise to cold HCl (3 N, 400 mL) over 45 min at −10° C. The resulting solution was extracted with 3×200 mL of DCM; the organic layers combined, dried over anhydrous Na₂SO₄, and concentrated under vacuum. This resulted in 53.5 g (crude) of the title compound as a yellow solid. The crude product was used in the next step.

### Step 2: 1,2,3,5,6,7-Hexahydro-s-indacen-1-one

Into a 1000-mL round-bottom flask was placed a solution of 3-chloro-1-(2,3-dihydro-1H-inden-5-yl)propan-1-one (53.5 g, 253 mmol) in conc. H₂SO₄ (300 mL). The resulting solution was stirred for 16 h at 55° C. and was then quenched by adding the reaction mixture carefully to 1500 mL of water/ice. The solids were collected by filtration and then was dried over infrared lamp for 24 h. This resulted in 37.4 g (85%) of the title compound as a yellow solid.

### Step 3: 1,2,3,5,6,7-Hexahydro-s-indacene

Into a 1000-mL round-bottom flask was placed a solution of 1,2,3,5,6,7-hexahydros-indacen-1-one (37.2 g, 216 mmol), MeOH (300 mL), and CH₃SO₃H (42 g, 437.5 mmol). Then Pd(OH)₂/C (20% wt., 8 g) was added. The flask was evacuated and flushed three times with hydrogen. The resulting solution was stirred for 16 h at RT under an atmosphere of hydrogen. The solids were filtered out. The resulting mixture was concentrated under vacuum. The residue was applied onto a silica gel column and eluted with ethyl acetate/petroleum ether (1:150 to 1:100). This resulted in 27.1 g (79%) of the title compound as a white solid.

### Step 4: 4-Bromo-1,2,3,5,6,7-hexahydro-s-indacene

Into a 500-mL 3-necked round-bottom flask purged with and maintained under nitrogen was placed a solution of 1,2,3,5,6,7-hexahydro-s-indacene (15 g, 94.8 mmol) in CCl₄ (200 mL). Then 12 (1.2 g, 4.72 mmol) was added. This was followed by the addition of a solution of Br₂ (16 g, 100 mmol) in CCl₄ (50 mL) dropwise with stirring at 0° C. in 10 min. The resulting solution was stirred for 2 h at 0° C. The reaction was then quenched by the addition of 150 mL of NH₄Cl (sat.). The resulting solution was extracted with 3×150 mL of DCM and the organic layers combined and dried over anhydrous Na₂SO₄, then concentrated under vacuum. The crude product was applied onto a silica gel column and eluted with ethyl acetate/petroleum ether. This resulted in 18.0 g (80%) of the title compound as yellow oil. ¹H NMR (300 MHz, DMSO-d₆) δ 7.02 (s, 1H), 2.95-2.75 (m, 8H), 2.03-2.01 (m, 4H).

### Step 5: Tert-butyl 2-(1,2,3,5,6,7-hexahydro-s-indacen-4-yl)acetate

Into a 100-mL round-bottom flask purged with and maintained under nitrogen was placed a solution of 4-bromo-1,2,3,5,6,7-hexahydro-s-indacene (1 g, 4.2 mmol) in THF (20 mL). Then X-phos (200 mg, 0.42 mmol) and Pd₂(dba)₃CHCl₃ (220 mg, 0.21 mmol) were added. The resulting solution was stirred for 10 min at RT. This was followed by the addition of tert-butyl 2-(bromozincio)acetate (2.2 g, 8.45 mmol). The resulting solution was stirred for 4 h at 80° C. and was then quenched by the addition of 50 mL of NH₄Cl (sat.). The resulting solution was extracted with 3×100 mL of DCM. The organic layers were combined, dried over anhydrous Na₂SO₄, and concentrated under vacuum. This resulted in 1.4 g (crude) of the title compound as brown oil. ¹H NMR (400 MHz, DMSO-d₆) δ 6.96 (s, 1H), 3.47 (s, 2H), 2.80-2.78 (m, 8H), 2.01-1.99 (m, 4H), 1.39 (s, 9H).

### Step 6: 2-(1,2,3,5,6,7-hexahydro-s-indacen-4-yl)acetic Acid

Into a 40-mL sealed tube was placed a solution of tert-butyl 2-(1,2,3,5,6,7-hexahydro-s-indacen-4-yl)acetate (1.4 g, 5.14 mmol) in 6 M sodium hydroxide/MeOH (4/6 mL). The resulting solution was stirred for 16 h at 100° C. The reaction was then quenched by the addition of 20 mL of water. The resulting solution was extracted with 2×30 mL of DCM and the aqueous layers combined. The pH value of the solution was adjusted to 2 with hydrogen chloride (1 N). The resulting solution was extracted with 3×50 mL of ethyl acetate and the organic layers combined and dried over anhydrous Na₂SO₄, then concentrated under vacuum. This resulted in 180 mg (19.8%, 2 steps) of the title compound as a yellow solid. MS-ESI. 215.1 (M−1).

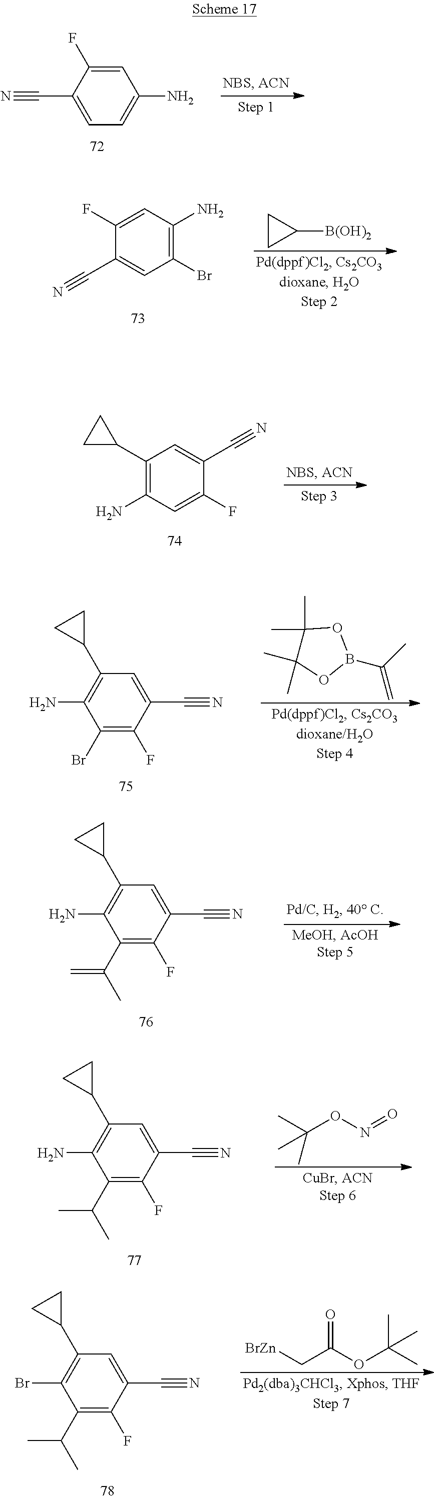

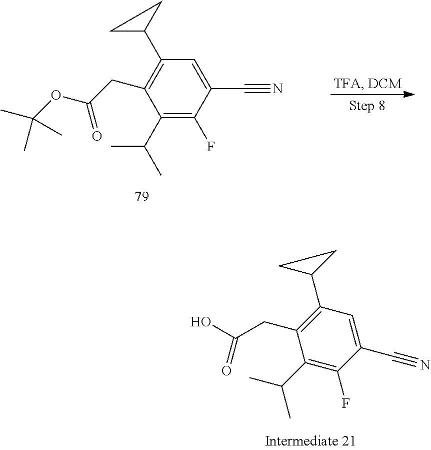 [SMILES: CC(C)c1c(F)c(C#N)cc(C2CC2)c1CC(=O)OC(C)(C)C ; CC(C)c1c(F)c(C#N)cc(C2CC2)c1CC(=O)O]

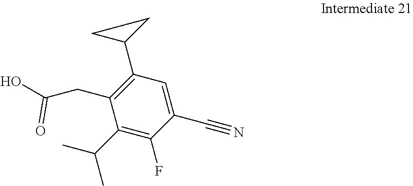 [SMILES: CC(C)c1c(F)c(C#N)cc(C2CC2)c1CC(=O)O]

### 2-(4-Cyano-6-cyclopropyl-3-fluoro-2-isopropylphenyl)acetic Acid

### Step 1: 4-Amino-5-bromo-2-fluorobenzonitrile

Into a 250-mL round-bottom flask was placed a solution of 4-amino-2-fluorobenzonitrile (9 g, 66.1 mmol) in ACN (120 mL). Then NBS (12.4 g, 69.7 mmol) was added. The resulting solution was stirred overnight at 80° C. and then was concentrated under vacuum. The residue was applied onto a silica gel column and eluted with ethyl acetate/petroleum ether (1:20 to 1:10). This resulted in 10.9 g (77%) of the title compound as a yellow solid. MS-ESI: 215.0/217.0 (M+1). ¹H NMR (300 MHz, DMSO-d₆) δ 7.89 (d, J=6.0 Hz, 1H), 6.69 (br s, 2H), 6.63 (d, J=12.0 Hz, 1H).

### Step 2: 4-Amino-5-cyclopropyl-2-fluorobenzonitrile

Into a 250-mL round-bottom flask purged with and maintained under nitrogen was placed 4-amino-5-bromo-2-fluorobenzonitrile (6.37 g, 29.6 mmol), 1,4-dioxane (70 mL), water (10 mL), Cs₂CO₃ (9.7 g, 29.8 mmol), cyclopropylboronic acid (3.8 g, 44.2 mmol), and Pd(dppf)Cl₂ (1.08 g, 1.48 mmol). The resulting solution was stirred overnight at 90° C. and then was concentrated under vacuum. The residue was applied onto a silica gel column and eluted with ethyl acetate/petroleum ether (1:10 to 1:5). This resulted in 5.03 g (96%) of the title compound as a yellow solid. MS-ESI: 177.1 (M+1).

### Step 3: 4-Amino-3-bromo-5-cyclopropyl-2-fluorobenzonitrile

Into a 250-mL round-bottom flask was placed 4-amino-5-cyclopropyl-2-fluorobenzonitrile (5.03 g, 28.7 mmol), ACN (50 mL), and NBS (5.6 g, 31.5 mmol). The resulting solution was stirred overnight at 80° C. and then was concentrated under vacuum. The residue was applied onto a silica gel column and eluted with ethyl acetate/petroleum ether (1:10 to 1:5). This resulted in 6.972 g (96%) of the title compound as a yellow solid. MS-ESI: 255.0/257.0 (M+1).

### Step 4: 4-Amino-5-cyclopropyl-2-fluoro-3-(prop-1-en-2-yl)benzonitrile

Into a 250-mL round-bottom flask purged with and maintained under nitrogen was placed 4-amino-3-bromo-5-cyclopropyl-2-fluorobenzonitrile (6.972 g, 27.33 mmol), 1,4-dioxane (120 mL), water (20 mL), 4,4,5,5-tetramethyl-2-(prop-1-en-2-yl)-1,3,2-dioxaborolane (6.9 g, 41.0 mmol), Cs₂CO₃ (13.4 g, 41.0 mmol), and Pd(dppf)Cl₂ (0.4 g, 0.55 mmol). The resulting solution was stirred overnight at 80° C. and was then concentrated under vacuum. The residue was applied onto a silica gel column and eluted with ethyl acetate/petroleum ether (1:10 to 1:5). This resulted in 4.73 g (80%) of the title compound as a yellow solid. MS-ESI: 217.1 (M+1).

### Step 5: 4-Amino-5-cyclopropyl-2-fluoro-3-isopropylbenzonitrile

Into a 250-mL round-bottom flask was placed 4-amino-5-cyclopropyl-2-fluoro-3-(prop-1-en-2-yl)benzonitrile (4.73 g, 21.97 mmol), MeOH (100 mL), and AcOH (0.5 mL). Then Pd/C (10% wt, 500 mg) was added. The flask was evacuated and flushed three times with hydrogen. The resulting solution was stirred for 4 h at 40° C. under an atmosphere of hydrogen. The solids were filtered out. The filtrate was concentrated under vacuum. This resulted in 4.71 g (99%) of the title compound as a light yellow solid. MS-ESI: 219.1 (M+1).

Steps 6-8 used similar procedures for converting compound 46 to Intermediate 14 shown in Scheme 13 to afford Intermediate 21. MS-ESI: 260.1 (M−1).

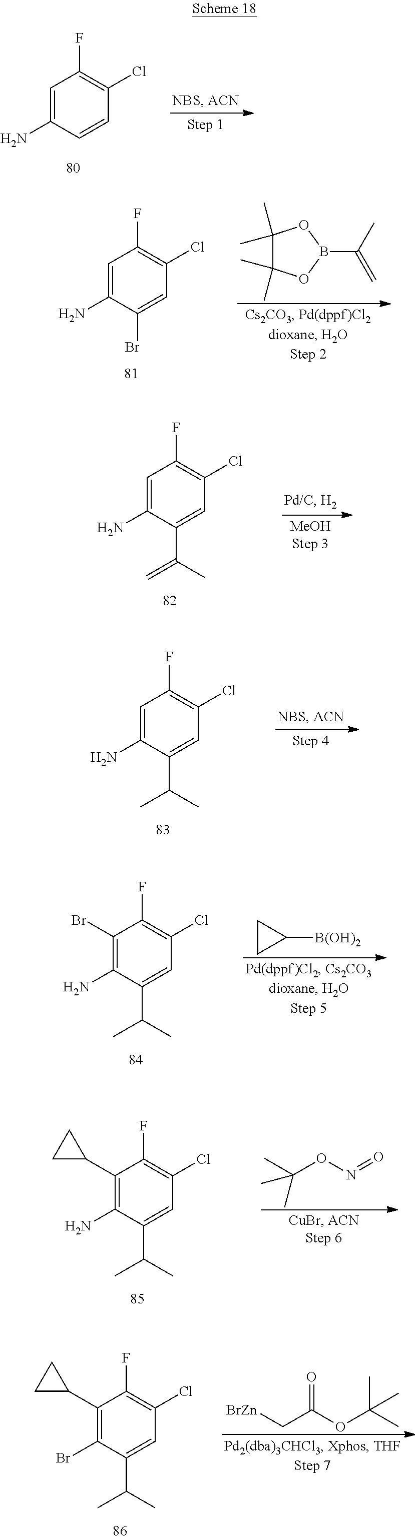

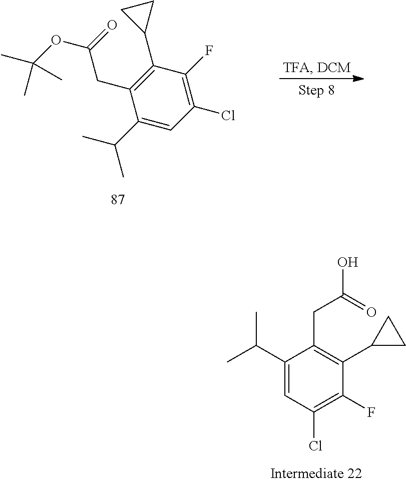 [SMILES: CC(C)c1cc(Cl)c(F)c(C2CC2)c1CC(=O)OC(C)(C)C ; CC(C)c1cc(Cl)c(F)c(C2CC2)c1CC(=O)O]

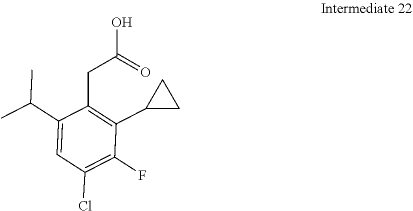 [SMILES: CC(C)c1cc(Cl)c(F)c(C2CC2)c1CC(=O)O]

### 2-(4-Chloro-2-cyclopropyl-3-fluoro-6-isopropylphenyl)acetic Acid

### Step 1: 2-Bromo-4-chloro-5-fluorobenzenamine

Into a 1000-mL round-bottom flask was placed 4-chloro-3-fluorobenzenamine (20 g, 137 mmol, ACN (500 mL), and NBS (21.9 g, 123 mmol). The resulting solution was stirred overnight at RT and then was concentrated under vacuum. The residue was applied onto a silica gel column and eluted with ethyl acetate/petroleum ether (1:150 to 1:100). This resulted in 26.3 g (85%) of the title compound as a white solid. MS-ESI: 225.9/223.9/227.9 (M+1). ¹H NMR (300 MHz, CDCl₃-d) δ 7.44 (d, J=8.0 Hz, 1H), 6.59 (d, J=8.8 Hz, 1H), 4.21 (s, 2H).

### Step 2: 4-Chloro-5-fluoro-2-(prop-1-en-2-yl)benzenamine

Into a 1000-mL round-bottom flask purged with and maintained under nitrogen was placed 2-bromo-4-chloro-5-fluorobenzenamine (26.3 g, 117 mmol), 1,4-dioxane (500 mL), water (50 mL), 4,4,5,5-tetramethyl-2-(prop-1-en-2-yl)-1,3,2-dioxaborolane (23.7 g, 141 mmol), Cs₂CO₃ (76.6 g, 235 mmol), and Pd(dppf)Cl₂ (1.71 g, 2.34 mmol). The resulting solution was stirred overnight at 90° C. and was then concentrated under vacuum. The residue was applied onto a silica gel column and eluted with ethyl acetate/petroleum ether (1:150 to 1:100). This resulted in 12.6 g (58%) of the title compound as brown oil. MS-ESI: 186.0/188.0 (M+1).

### Step 3: 4-Chloro-5-fluoro-2-isopropylbenzenamine

Into a 500-mL round-bottom flask was placed 4-chloro-5-fluoro-2-(prop-1-en-2-yl)benzenamine (12.6 g, 67.88 mmol) in MeOH (250 mL). Then Pd/C (10% wt, 1.2 g) was added. The flask was evacuated and flushed three times with hydrogen. The resulting solution was stirred for 3 h at RT under an atmosphere of hydrogen. The solids were filtered out. The filtrate was concentrated under vacuum. This resulted in 12.5 g (98%) of the title compound as light yellow oil. MS-ESI: 188.1/190.1 (M+1).

### Step 4: 2-Bromo-4-chloro-3-fluoro-6-isopropylbenzenamine

Into a 500-mL round-bottom flask was placed 4-chloro-5-fluoro-2-isopropylbenzenamine (6 g, 32.0 mmol), ACN (200 mL), and NBS (6.25 g, 35.1 mmol). The resulting solution was stirred for 3 h at RT and then was concentrated under vacuum. The residue was applied onto a silica gel column and eluted with ethyl acetate/petroleum ether (1:150 to 1:100). This resulted in 8 g (94%) of the title compound as brown oil. MS-ESI: 268.0/266.0/270.0 (M+1).

### Step 5: 4-Chloro-2-cyclopropyl-3-fluoro-6-isopropylbenzenamine

Into a 100-mL round-bottom flask purged with and maintained under nitrogen was placed 2-bromo-4-chloro-3-fluoro-6-isopropylbenzenamine (2.9 g, 10.9 mmol), 1,4-dioxane (40 mL), water (8 mL), cyclopropylboronic acid (1.12 g, 13.0 mmol), Cs₂CO₃ (7.08 g, 21.7 mmol), and Pd(dppf)Cl₂ (795 mg, 1.09 mmol). The resulting solution was stirred for 3 h at 90° C. and then was concentrated under vacuum. The residue was applied onto a silica gel column and eluted with ethyl acetate/petroleum ether (1:80 to 1:50). This resulted in 1.1 g (44%) of the title compound as light brown oil. MS-ESI: 228.1/230.1 (M+1).

Steps 6-8 used similar procedures for converting compound 46 to Intermediate 14 shown in Scheme 13 to afford Intermediate 22. MS-ESI: 271.1/273.1 (M−1).

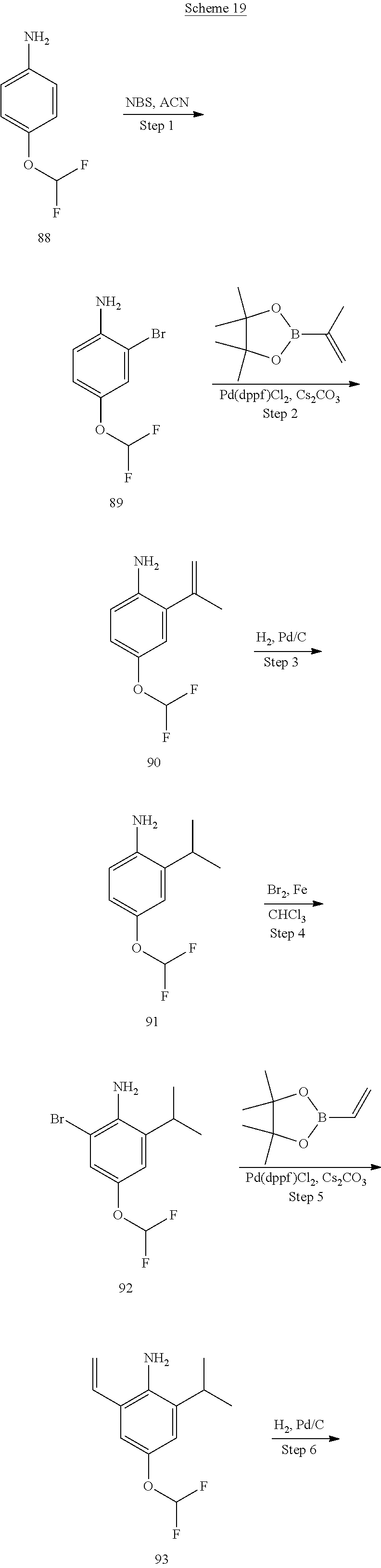

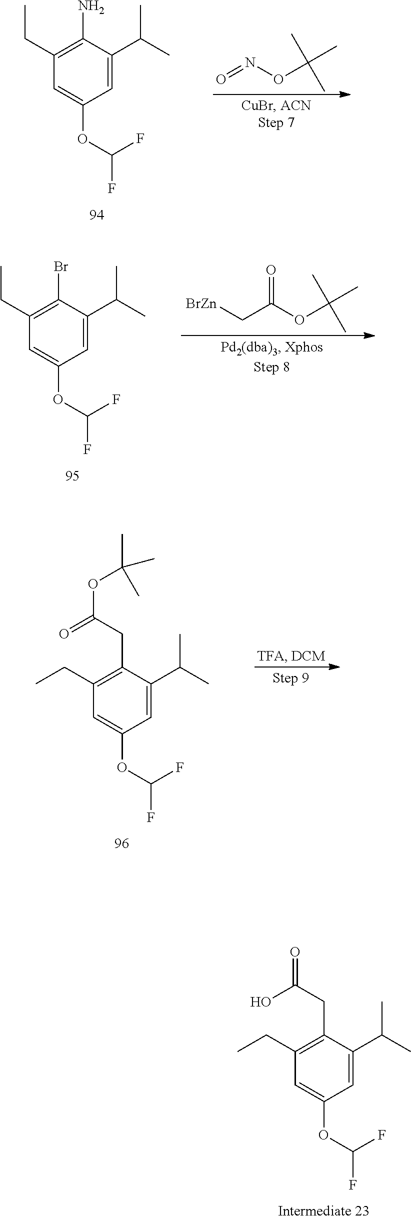

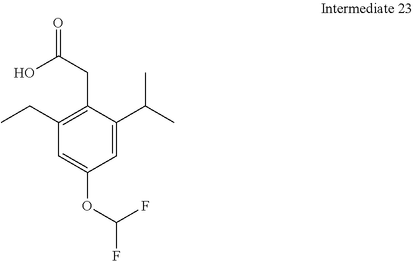 [SMILES: CCc1cc(OC(F)F)cc(C(C)C)c1CC(=O)O]

### 2-(4-(Difluoromethoxy)-2-ethyl-6-isopropylphenyl)acetic Acid

Steps 1-3 used similar procedures for converting compound 80 to compound 83 shown in Scheme 18 to afford compound 91. MS-ESI. 202.1 (M+1).

### Step 4: 2-Bromo-4-(difluoromethoxy)-6-isopropylbenzenamine

Into a 250-mL round-bottom flask was placed a mixture of 4-(difluoromethoxy)-2-isopropylbenzenamine (2.01 g, 10 mmol) and iron powder (1.12 g, 20 mmol) in CHCl₃ (50 mL). To this was added bromine (1.23 mL, 24 mmol). The resulting solution was stirred for 6 h at RT and diluted with water (200 mL). The mixture was extracted with 3×50 mL of ethyl acetate. The organic layers were combined, dried over Na₂SO₄, and then concentrated under vacuum. The residue was applied onto a silica gel column and eluted with ethyl acetate/petroleum ether (1:100 to 1:10). This resulted in 2.24 g (80%) of the title compound as a yellow solid. MS-ESI: 280.0/282.0 (M+1).

Steps 5-9 used similar procedures for converting compound 44 to Intermediate 14 shown in Scheme 13 to afford Intermediate 23. MS-ESI: 271.1 (M−1).

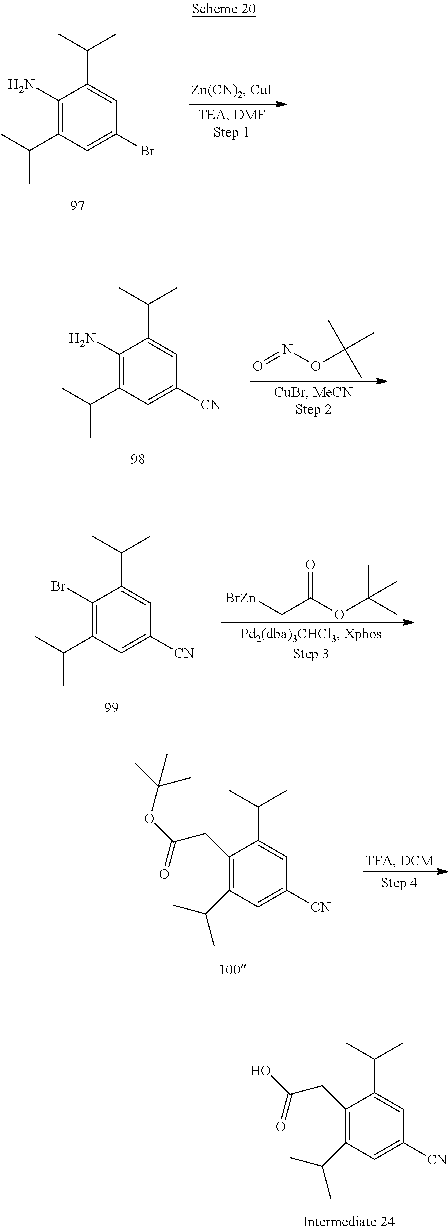

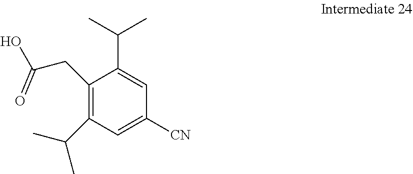 [SMILES: CC(C)c1cc(C#N)cc(C(C)C)c1CC(=O)O]

### 2-(4-Cyano-2,6-diisopropylphenyl)acetic Acid

### Step 1: 4-Amino-3,5-diisopropylbenzonitrile

Into a 100-mL round-bottom flask purged with and maintained under nitrogen was placed a solution of 4-bromo-2,6-diisopropylbenzenamine (commercially available, 5.1 g, 19.9 mmol) in DMF (30 mL). To the solution were added Zn(CN)₂ (2.80 g, 23.9 mmol), CuI (380 mg, 2.00 mmol), and TEA (3.0 g, 29.9 mmol). The resulting solution was stirred for 16 h at 120° C. and then was diluted with 30 mL of water. The solution was extracted with 3×30 mL of ethyl acetate and the combined organic layers were concentrated under vacuum. The residue was applied onto a silica gel column and eluted with ethyl acetate/petroleum ether (1:30 to 1:20). This resulted in 2.4 g (60%) of the title compound as a yellow solid. MS-ESI: 203.1 (M+1).

Steps 2-4 used similar procedures for converting compound 46 to Intermediate 14 shown in Scheme 13 to afford Intermediate 24. MS-ESI: 244.1 (M−1).

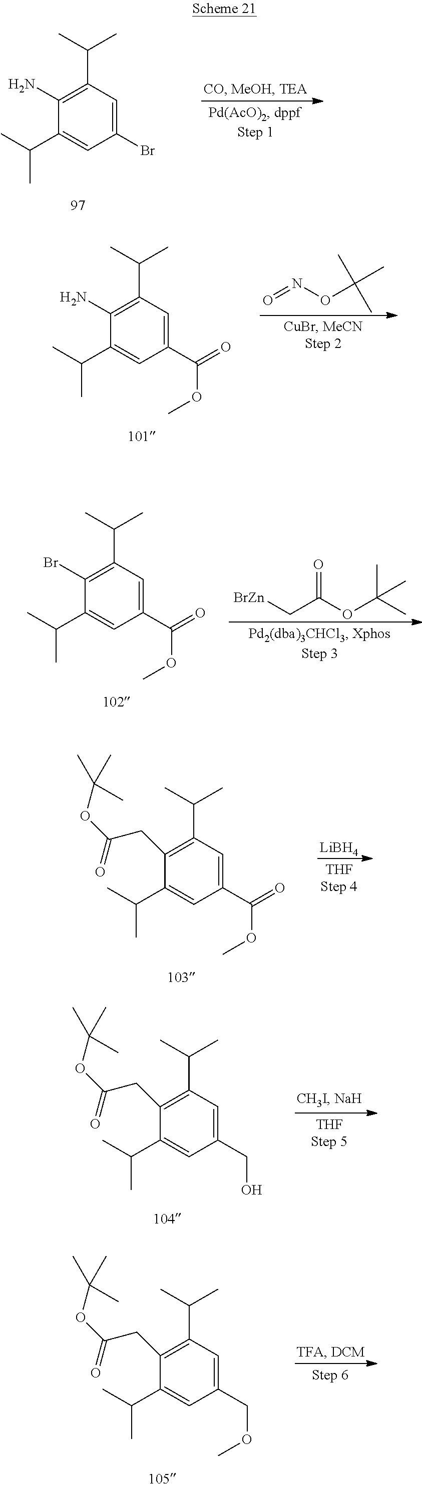

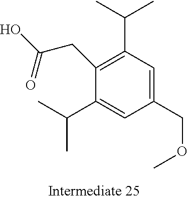 [SMILES: COCc1cc(C(C)C)c(CC(=O)O)c(C(C)C)c1]

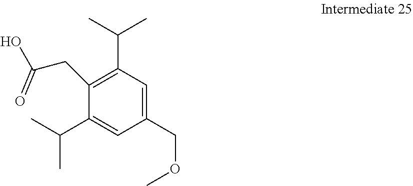 [SMILES: COCc1cc(C(C)C)c(CC(=O)O)c(C(C)C)c1]

### 2-(2,6-diisopropyl-4-(methoxymethyl)phenyl)acetic Acid

### Step 1: Methyl 4-amino-3,5-diisopropylbenzoate

Into a 1-L autoclave was placed a solution of 4-bromo-2,6-diisopropylbenzenamine (10 g, 39 mmol) in MeOH (300 mL). To the solution were added Pd(OAc)₂ (1.75 g, 7.8 mmol), dppf (4.3 g, 7.8 mmol), and TEA (20 g, 195 mmol). After sealing the autoclave, the gas was exchanged with CO for 3 times. The reaction was stirred at 120° C. for overnight. After cooling the reaction mixture, the gas was exchanged with N₂, the reaction was concentrated and diluted with water (300 mL). The resulting solution was extracted with EtOAc (3×200 mL). The combined organic layers were dried over anhydrous Na₂SO₄ and concentrated. The residue was purified on SiO₂-gel column and eluted with ethyl acetate/petroleum ether (1:10 to 1:5). This resulted in 5.6 g (62%) of the title compound as a brown oil. MS-ESI: 236.2 (M+1)

Steps 2 and 3 used similar procedures for converting compound 46 to 48, shown in Scheme 13 to afford compound 103″ as colorless oil. 335.2 (M+1)

### Step 4: Tert-butyl 2-(4-(hydroxymethyl)-2,6-diisopropylphenyl)acetate

Into a 100 mL round bottom flask was placed a solution of methyl 4-(2-tert-butoxy-2-oxoethyl)-3,5-diisopropylbenzoate (2 g, 6.0 mmol) in THF (25 mL). LiBH₄ (264 mg, 12.0 mmol) was added to the mixture at 0° C. in portions, and the mixture was stirred at 0° C. for 1 h. The reaction was quenched with ice-water (20 mL). The solution was extracted with EtOAc (3×100 mL); the combined organic layers were dried over anhydrous Na₂SO₄ and concentrated under vacuum. The residue was purified with SiO₂-gel column and eluted with ethyl acetate/petroleum ether (1:5 to 1:2). This resulted in 1.1 g (60%) of the title compound as a white solid. MS-ESI: 307.2 (M+1).

### Step 5: Tert-butyl 2-(2,6-diisopropyl-4-(methoxymethyl)phenyl)acetate

Into a 100 mL round bottom flask was placed a solution of tert-butyl 2-(4-(hydroxymethyl)-2,6-diisopropylphenyl)acetate (1.1 g, 3.6 mmol) in THF (20 mL). NaH (60% wt., 173 mg, 4.3 mmol) was added to the mixture at 0° C. in portions, and the mixture was stirred at 0° C. for 30 min. MeI (1.0 g, 7.2 mmol) was added to the mixture dropwise at 0° C.; the resulting mixture was stirred at RT for overnight. The reaction was quenched with ice-water (20 mL) and extracted with EtOAc (3×100 mL). The combined organic layer were dried over Na₂SO₄ and concentrated under vacuum. The residue was purified with SiO₂-gel column and eluted with ethyl acetate/petroleum ether (1:10-1:5). This resulted in 1.1 g (95%) of title compound as a colorless oil. MS-ESI: 321.2 (M+1).

### Step 6: 2-(2,6-Diisopropyl-4-(methoxymethyl)phenyl)acetic Acid

Into a 50-mL round-bottom flask was placed a solution of tert-butyl 2-[4-fluoro-2,6-bis(propan-2-yl)phenyl]acetate (1.1 g, 3.4 mmol) in DCM (10 mL) and TFA (10 mL). The resulting solution was stirred for 3 h at RT and then was concentrated under vacuum. This resulted in 1.0 g (crude) of the title compound as a light yellow solid. MS-ESI: 263.2 (M−1).

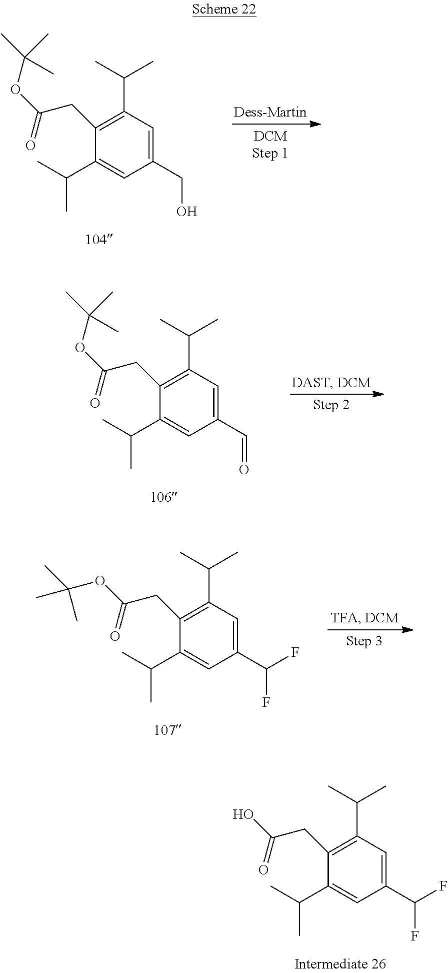 [SMILES: CC(C)c1cc(CO)cc(C(C)C)c1CC(=O)OC(C)(C)C ; CC(C)c1cc(C=O)cc(C(C)C)c1CC(=O)OC(C)(C)C ; CC(C)c1cc(C(F)F)cc(C(C)C)c1CC(=O)OC(C)(C)C ; CC(C)c1cc(C(F)F)cc(C(C)C)c1CC(=O)O]

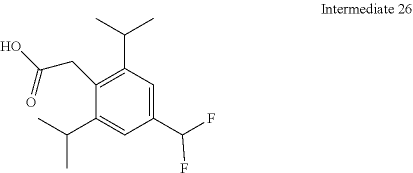 [SMILES: CC(C)c1cc(C(F)F)cc(C(C)C)c1CC(=O)O]

### 2-(4-(Difluoromethyl)-2,6-diisopropylphenyl)acetic Acid

### Step 1: Tert-butyl 2-(4-formyl-2,6-diisopropylphenyl)acetate

Into a 100 mL round bottom flask was placed a solution of tert-butyl 2-(4-(hydroxymethyl)-2,6-diisopropylphenyl)acetate (1.1 g, 3.6 mmol) in DCM (20 mL). Dess-Martin Periodinane (2.29 g, 5.4 mmol) was added to the mixture at 0° C. in portions. The mixture was stirred at RT overnight, after which the reaction was quenched with ice-water (20 mL) and extracted with DCM (3×50 mL). The combined organic layers were dried over anhydrous Na₂SO₄ and concentrated under vacuum. The residue was purified on a SiO₂-gel column and eluted with ethyl acetate/petroleum ether (1:20-1:10). This resulted in 0.98 g (90%) of title compound as a yellow solid. MS-ESI: 305.2 (M+1).

### Step 2: Tert-butyl 2-(4-(difluoromethyl)-2,6-diisopropylphenyl)acetate

Into a 100 mL round bottom flask was placed a solution of tert-butyl 2-(4-formyl-2,6-diisopropylphenyl)acetate (912 mg, 3.0 mmol) in DCM (15 mL). DAST (2.41 g, 15 mmol) was added to the mixture at 0° C. in portions. The mixture was stirred at RT overnight, after which the reaction was quenched with water (10 mL) and extracted with DCM (3×30 mL). The combined organic layers were dried over Na₂SO₄ and concentrated under vacuum. The residue was purified with SiO₂-gel column and eluted with ethyl acetate/petroleum ether (1:20-1:15). This resulted in 586 mg (60%) of title compound as a yellow solid. MS-ESI: 327.2 (M+1).

Steps 3 used similar procedures for converting compound 105″ to Intermediate 25 shown in Scheme 21 to afford Intermediate 26. MS-ESI: 269.1 (M−1).

Schemes of Sulfonimidoylamide Intermediates: Schemes 23-30 illustrate the preparation of sulfonimidoylamide intermediates.

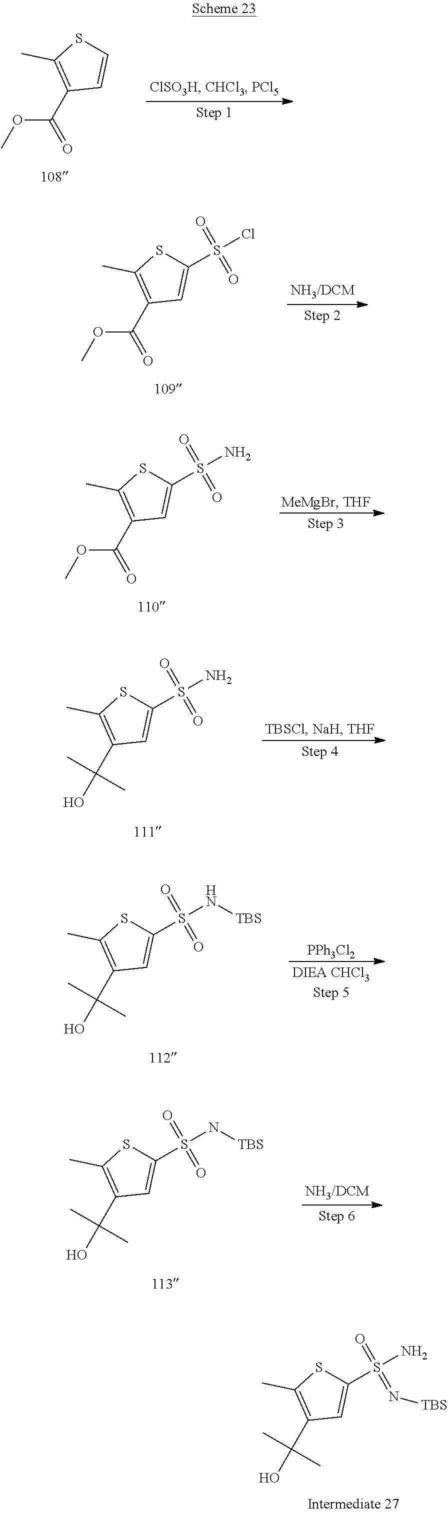

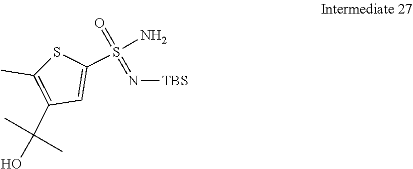

### N′-(tert-butyldimethylsilyl)-4-(2-hydroxypropan-2-yl)-5-methylthiophene-2-sulfonimidamide

### Step 1: Methyl 5-(chlorosulfonyl)-2-methylthiophene-3-carboxylate

Into a 250-mL round-bottom flask, was placed methyl 2-methylthiophene-3-carboxylate (5.0 g, 32.0 mmol), CHCl₃ (70 mL). This was followed by the addition of ClSO₂OH (5.6 g, 48.0 mmol) dropwise with stirring. To this was added PCl₅ (13.3 g, 64.0 mmol) with stirring. The resulting solution was stirred for 2 h at 60° C. in an oil bath. The reaction was then quenched by the addition of 150 mL of water/ice. The resulting solution was extracted with 3×80 ml of dichloromethane, dried over anhydrous sodium sulfate, and concentrated. This resulted in 5.2 g (63.8%) of the title compound as a yellow solid.

### Step 2: Methyl 2-methyl-5-sulfamoylthiophene-3-carboxylate

Into a 250-mL round-bottom flask, was placed methyl 5-(chlorosulfonyl)-2-methylthiophene-3-carboxylate (5.2 g, 20.4 mmol) in DCM (50 mL), to this solution was added NH₃/DCM (50 mL, sat.) dropwise with stirring. The resulting solution was stirred for 2 h at 40° C. in an oil bath. The resulting mixture was concentrated. The residue was eluted from a silica gel with ethyl acetate/petroleum ether (2:3). This resulted in 4.6 g (95.8%) of the title compound as a yellow solid. MS-ESI: 236 [M+1].

### Step 3: 4-(2-Hydroxypropan-2-yl)-5-methylthiophene-2-sulfonamide

Into a 250-mL 3-necked round-bottom flask purged and maintained with an inert atmosphere of nitrogen, was placed methyl 2-methyl-5-sulfamoylthiophene-3-carboxylate (4.6 g, 19.5 mmol) in THF (100 mL). This was followed by the addition of MeMgBr (29 mL, 87 mmol, 3M) dropwise with stirring at 0° C. in an ice bath. The resulting solution was stirred for 2 h at RT. The pH value of the solution was adjusted to 5 with HCl (2 M). The resulting solution was extracted with 3×100 ml of ethyl acetate, dried over anhydrous sodium sulfate, and concentrated. The residue was eluted from silica gel with ethyl acetate/petroleum ether (1:2). This resulted in 1.3 g (28.2%) of the title compound as a light yellow solid. MS-ESI: 236 [M+1].

### Step 4: N-(tert-butyldimethylsilyl)-4-(2-hydroxypropan-2-yl)-5-methylthiophene-2-sulfonamide

Into a 100-mL round-bottom flask, was placed 4-(2-hydroxypropan-2-yl)-5-methylthiophene-2-sulfonamide (1.3 g, 5.52 mmol) in THF (40 mL). To this solution was added NaH (60% wt. oil dispersion, 442 mg, 11.1 mmol) in portions with stirring at 0° C. This was followed by the addition of TBSCl (1.25 g, 8.29 mmol). The resulting solution was stirred for 1 h at RT. The reaction was then quenched by the addition of 50 mL of NH₄Cl solution. The resulting solution was extracted with 3×50 ml of ethyl acetate, dried over anhydrous sodium sulfate, and concentrated. The residue was eluted from silica gel with ethyl acetate/petroleum ether (1:2). This resulted in 1.2 g (62.1%) of the title compound as a white solid. MS-ESI: 350[M+1].

### Steps 5 and 6: N′-(tert-butyldimethylsilyl)-4-(2-hydroxypropan-2-yl)-5-methylthiophene-2-sulfonimidamide

Into a 100-mL 3-necked round-bottom flask purged and maintained with an inert atmosphere of nitrogen, was placed a solution of PPh₃Cl₂ (3.51 g, 10.5 mmol) in CHCl₃ (40 mL). This was followed by the addition of DIEA (1.77 g, 13.7 mmol) dropwise with stirring at RT. The resulting solution was stirred for 10 min at RT and the reaction mixture was cooled to 0° C. To this was added a solution of N-(tert-butyldimethylsilyl)-4-(2-hydroxypropan-2-yl)-5-methylthiophene-2-sulfonamide (1.2 g, 3.43 mmol) in CHCl₃ (5 mL) dropwise with stirring at 0° C. The resulting solution was stirred for 30 min at 0° C. To the mixture was introduced NH₃ gas bubble for 15 min at 0° C. The resulting solution was stirred for 2 h at RT. The resulting solution was diluted with 50 mL of H₂O. The resulting solution was extracted with 3×100 ml of DCM and dried over anhydrous sodium sulfate and concentrated. The residue was eluted from silica gel with ethyl acetate/petroleum ether (1:1). This resulted in 930 mg (77.7%) of the title compound as a yellow solid. MS-ESI: 349 [M+1].

TABLE 12

| | | | Exact |
|---|---|---|---|
| Intermediate | | | Mass |
| # | Structure | IUPAC Name | [M + H]⁺ |
| Intermediate 28 | 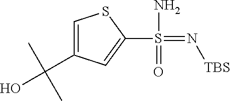 | N′-(tert-butyldimethylsily1)-4-(2-hydroxypropan-2-yl)thiophene-2- sulfonimidamide | 335 |

The Intermediates in the following Table were prepared using similar procedure as shown in Scheme 23 above for converting compound 108″ to Intermediate 27 starting from methyl thiophene-3-carboxylate.

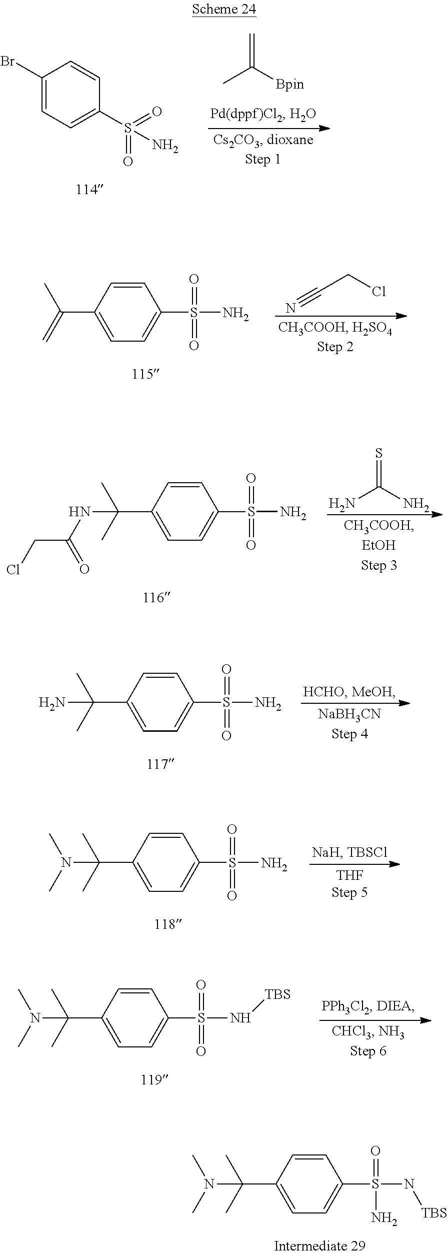

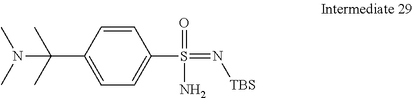

### N′-(tert-butyldimethylsilyl)-4-(2-(dimethylamino)propan-2-yl)benzenesulfonimidamide

### Step 1: 4-(Prop-1-en-2-yl)benzenesulfonamide

Into a 500-mL 3-necked round-bottom flask purged and maintained with an inert atmosphere of nitrogen, was placed a solution of 4-bromobenzene-1-sulfonamide (5.0 g, 21.2 mmol) in dioxane (75 mL) and H₂O (7.5 mL). To this solution was added 4,4, 5, 5-tetramethyl-2-(prop-1-en-2-yl)-1,3,2-dioxaborolane (7.83 g, 46.59 mmol), Pd(dppf)Cl₂ (1.5 g, 2.12 mmol) and Cs₂CO₃ (27.6 g, 84.7 mmol). The resulting solution was stirred for 2 h at 85° C. The resulting solution was diluted with 400 mL of water. The resulting solution was extracted with 2×500 mL of ethyl acetate and dried over anhydrous sodium sulfate and concentrated. The residue was eluted from silica gel with ethyl acetate/petroleum ether (1:3). This resulted in 4.7 g (98.1%) of the title compound as a yellow solid. MS-ESI: 198.1 [M+1].

### Step 2: 2-Chloro-N-(2-(4-sulfamoylphenyl)propan-2-yl)acetamide

Into a 1 L round-bottom flask, was placed a solution of 4-(prop-1-en-2-yl)benzene-1-sulfonamide (2.2 g, 11.2 mmol) in AcOH (280 mL). To the solution was added 2-chloroacetonitrile (16.8 g, 224 mmol). This was followed by the addition of H₂SO₄ (70 mL, 0.7 mmol) dropwise with stirring at 0° C. The resulting solution was stirred overnight at RT. The resulting solution was diluted with 500 mL of water/ice. The pH value of the solution was adjusted to 7 with a saturated solution of Na₂CO₃. The resulting solution was extracted with 3×1000 mL of DCM, dried over anhydrous sodium sulfate and concentrated. The residue was eluted from silica gel with ethyl acetate/petroleum ether (3:2). This resulted in 2.7 g (83.2%) of the crude title compound as a white solid. MS-ESI: 291.0 [M+1].

### Step 3: 4-(2-Aminopropan-2-yl)benzenesulfonamide

Into a 100-mL round-bottom flask, was placed a solution of 2-chloro-N-[2-(4-sulfamoylphenyl)propan-2-yl] acetamide (1.0 g, 3.44 mmol) in ethanol (30 mL) and AcOH (6.0 mL, 99.93 mmol). To the solution was added thiourea (314.2 mg, 4.13 mmol). The resulting solution was stirred for overnight at 85° C. The resulting mixture was concentrated. The resulting mixture was washed with 50 mL of ethanol. The solids were collected by filtration. The solid was dried under infra-red for 16 h. This resulted in 520 mg (70.56%) of the crude title compound as a white solid. MS-ESI: 215.1 [M+1].

### Step 4: 4-(2-(Dimethylamino)propan-2-yl)benzenesulfonamide

Into a 50-mL round-bottom flask, was placed a solution of 4-(2-aminopropan-2-yl)benzene-1-sulfonamide (500 mg, 2.33 mmol) in methanol (20 mL). This was followed by the addition of HCHO (140 mg, 4.67 mmol). The resulting solution was stirred for 30 min at RT. To this was added NaBH₃CN (439 mg, 7.0 mmol) in several batches at 0° C. The resulting solution was stirred for 1 h at RT. The reaction was then quenched by the addition of 50 mL of water. The resulting mixture was concentrated and washed with 20 mL of H₂O. The solids were collected by filtration and dried in an oven under reduced pressure at 50° C. This resulted in 300 mg (53.1%) of the crude title compound as a white solid. MS-ESI: 243.1[M+1].

### Step 5: N-(tert-butyldimethylsilyl)-4-(2-(dimethylamino)propan-2-yl)benzenesulfonamide

Into a 50-mL round-bottom flask, was placed a solution of 4-[2-(dimethylamino)propan-2-yl]benzene-1-sulfonamide (200 mg, 0.83 mmol) in THF (15 mL). This was followed by the addition of NaH (60% wt. oil dispersion, 66 mg, 1.65 mmol) at 0° C. The resulting solution was stirred for 10 min at RT. To this was added TBSCl (497 mg, 3.3 mmol). The resulting solution was stirred for 1 h at RT. The reaction was then quenched by the addition of 60 mL of water/ice. The resulting solution was extracted with 2×60 mL of ethyl acetate and dried over anhydrous sodium sulfate and concentrated. The residue was eluted from silica gel with ethyl acetate/petroleum ether (1:1). This resulted in 243 mg (82.5%) of the title compound as a white solid. MS-ESI: 357.2 [M+1].

### Step 6: N′-(tert-butyldimethylsilyl)-4-(2-(dimethylamino)propan-2-yl)benzenesulfonimidamide

Into a 250-mL 3-necked round-bottom flask purged and maintained with an inert atmosphere of nitrogen, was placed a solution of dichlorotriphenyl-λ5-phosphane (467 mg, 1.4 mmol) in CHCl₃ (30 mL). This was followed by the addition of DIEA (261 mg, 2.02 mmol) dropwise with stirring. The resulting solution was stirred for 15 min at RT, and the reaction system was cooled to 0° C. To this was added a solution of N-(tert-butyldimethylsilyl)-4-[2-(dimethylamino)propan-2-yl]benzene-1-sulfonamide (200 mg, 0.56 mmol) in CHCl₃ (10 mL) dropwise with stirring at 0° C. The resulting solution was stirred for 30 min at 0° C. To the mixture was added a solution of NH₃ in DCM (60 mL, sat.). The resulting solution was stirred for 2 h at RT. The resulting mixture was diluted with 80 mL of H₂O. The resulting solution was extracted with 2×100 mL of DCM and dried over anhydrous sodium sulfate and concentrated. The residue was eluted from silica gel with ethyl acetate/petroleum ether (1.4:1). This resulted in 140 mg (70.1%) of the title compound as a white solid. MS-ESI: 356.2 [M+1].

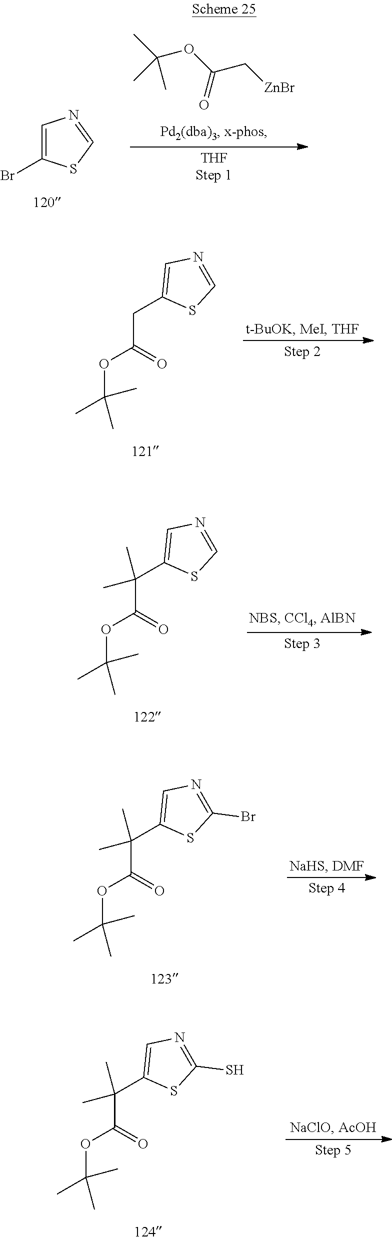

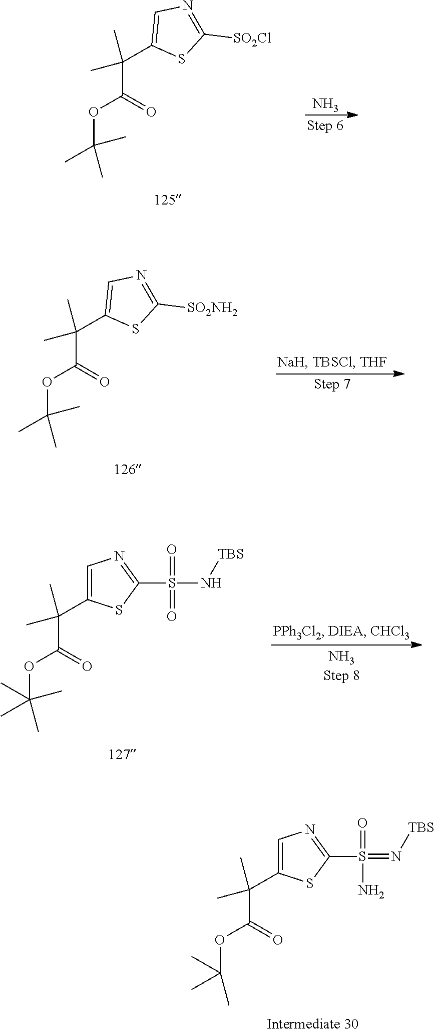

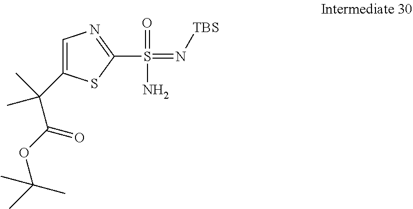

### Tert-butyl 2-(2-(N′-(tert-butyldimethylsilyl)sulfamidimidoyl)thiazol-5-yl)-2-methylpropanoate

### Step 1: Tert-butyl 2-(thiazol-5-yl)acetate

Into a 250-mL 3-necked round-bottom flask purged and maintained with an inert atmosphere of nitrogen, was placed a solution of 5-bromo-1,3-thiazole (3.0 g, 18.3 mmol) in THF (100 mL). This was followed by the addition of Pd₂(dba)₃ (947 mg, 0.91 mmol) and Xphos (1.05 g, 1.83 mmol). The resulting solution was stirred for 10 min at RT. To this was added tert-butyl 2-(bromozincio)acetate (9.5 g, 36.5 mmol). The resulting solution was stirred for 1.5 h at 60° C. The resulting mixture was diluted with 150 mL of H₂O. The resulting solution was extracted with 2×200 mL of ethyl acetate, dried over anhydrous sodium sulfate and concentrated. The residue was eluted from silica gel with ethyl acetate/petroleum ether (1:5). This resulted in 1.0 g (27.4%) of the title compound as a yellow liquid. MS-ESI: 200.1[M+1].

### Step 2: Tert-butyl 2-methyl-2-(thiazol-5-yl)propanoate

Into a 250-mL round-bottom flask, was placed a solution of tert-butyl 2-(1,3-thiazol-5-yl)acetate (1.0 g, 5.02 mmol) in THF (50 mL). To the solution were added t-BuOK (2.30 g, 20.4 mmol) and MeI (2.91 g, 20.4 mmol). The resulting solution was stirred for 1 h at RT. The reaction was then quenched by the addition of 200 mL of water. The resulting solution was extracted with 3×200 mL of ethyl acetate dried over anhydrous sodium sulfate and concentrated. The residue was eluted from silica gel with ethyl acetate/petroleum ether (1:5). This resulted in 1.05 g (92.0%) of the title compound as brown yellow oil. MS-ESI: 228.1 [M+1].

### Step 3: Tert-butyl 2-(2-bromothiazol-5-yl)-2-methylpropanoate

Into a 100-mL round-bottom flask purged and maintained with an inert atmosphere of nitrogen, was placed a solution of tert-butyl 2-methyl-2-(1,3-thiazol-5-yl)propanoate (500 mg, 2.2 mmol) in CCl₄ (30 mL). To the solution were added NBS (783 mg, 4.4 mmol) and AIBN (72.2 mg, 0.44 mmol). The resulting solution was stirred for 5 h at 70° C. The reaction was then quenched by the addition of 60 mL of water. The resulting solution was extracted with 2×100 mL of DCM, dried over anhydrous sodium sulfate, and concentrated. The residue was eluted from silica gel with ethyl acetate/petroleum ether (1:6). This resulted in 450 mg (66.9%) of the title compound as yellow oil. MS-ESI: 306.0 [M+1].

### Step 4: Tert-butyl 2-(2-mercaptothiazol-5-yl)-2-methylpropanoate

Into a 50-mL round-bottom flask, was placed a solution of tert-butyl 2-(2-bromo-1,3-thiazol-5-yl)-2-methylpropanoate (450 mg, 1.5 mmol) in DMF (10 mL). To the solution was added NaSH (2.97 g, 30 mmol). The resulting solution was stirred overnight at 100° C. The pH value was adjusted to 6 with 1M HCl. The resulting solution was washed with 2×25 mL of H₂O and extracted with 2×50 mL of ethyl acetate. The organic layers were combined and dried over anhydrous sodium sulfate. The residue was eluted from silica gel with ethyl acetate/petroleum ether (1:1). This resulted in 350 mg (91.6%) of the title compound as yellow oil. MS-ESI: 260.1 [M+1].

### Step 5: Tert-butyl 2-(2-(chlorosulfonyl)thiazol-5-yl)-2-methylpropanoate

Into a 25-mL round-bottom flask, was placed a solution of tert-butyl 2-methyl-2-(2-sulfanyl-1,3-thiazol-5-yl)propanoate (350 mg, 1.35 mmol) in AcOH (10 mL) at 0° C. To this was added NaClO (10% wt., 5.03 g, 67.4 mmol) dropwise with stirring at 0° C. The resulting solution was stirred for 90 min at RT. The resulting mixture was diluted with 2×100 mL of H₂O. The resulting solution was extracted with 150 mL of DCM, the combined organic layer was dried over anhydrous sodium sulfate and concentrated. This resulted in 100 mg (56.8%) of the title compound as yellow oil.

### Step 6: Tert-butyl 2-methyl-2-(2-sulfamoylthiazol-5-yl)propanoate

Into a 50-mL round-bottom flask, was placed a solution of tert-butyl 2-[2-(chlorosulfonyl)-1,3-thiazol-5-yl]-2-methylpropanoate (100 mg, 0.31 mmol) in DCM (5 mL). To the above solution NH₃ (g) was introduced. The resulting solution was stirred for 20 min at RT. The resulting mixture was concentrated. The residue was eluted from silica gel with ethyl acetate/petroleum ether (3:4). This resulted in 90 mg (95.7%) of the title compound as a white solid. MS-ESI: 307.1 [M+1].

### Step 7: Tert-butyl 2-(2-(N-(tert-butyldimethylsilyl)sulfamoyl)thiazol-5-yl)-2-methylpropanoate

Into a 25-mL round-bottom flask, was placed a solution of tert-butyl 2-methyl-2-(2-sulfamoyl-1,3-thiazol-5-yl)propanoate (50 mg, 0.16 mmol) in THF (5 mL). This was followed by the addition of NaH (60% wt. oil dispersion, 9.6 mg, 0.24 mmol) at 0° C. To this was added TBSCl (49.2 mg, 0.33 mmol). The resulting solution was stirred for 40 min at RT. The reaction was then quenched by the addition of 30 mL of water/ice. The resulting solution was extracted with 2×50 mL of ethyl acetate, dried over anhydrous sodium sulfate and concentrated. The residue was eluted from silica gel with ethyl acetate/petroleum ether (1:3). This resulted in 120 mg (97.1%) of the title compound as a white solid. MS-ESI: 421.2 [M+1].

### Step 8: Tert-butyl 2-(2-(N′-(tert-butyldimethylsilyl)sulfamidimidoyl)thiazol-5-yl)-2-methylpropanoate

Into a 50-mL 3-necked round-bottom flask purged and maintained with an inert atmosphere of nitrogen, was placed a solution of PPh₃Cl₂ (236 mg, 0.71 mmol) in CHCl₃ (15 mL). This was followed by the addition of DIEA (147 mg, 1.14 mmol) dropwise with stirring at 0° C. The resulting solution was stirred for 15 min at RT. To this was added a solution of tert-butyl 2-[2-[(tert-butyldimethylsilyl)sulfamoyl]-1,3-thiazol-5-yl]-2-methylpropanoate (120 mg, 0.29 mmol) in CHCl₃ (4 mL) dropwise with stirring at 0° C. The resulting solution was stirred for 30 min at 0° C. To the above solution was introduced NH₃(g). The resulting solution was stirred for 1 h at RT. The resulting mixture was diluted with 50 mL of H₂O. The resulting solution was extracted with 2×75 mL of DCM, the combined organic layer was dried over anhydrous sodium sulfate and concentrated. The residue was eluted from silica gel with ethyl acetate/petroleum ether (1:6). This resulted in 80 mg (66.6%) of the title compound as a white solid. MS-ESI: 420.2 [M+1].

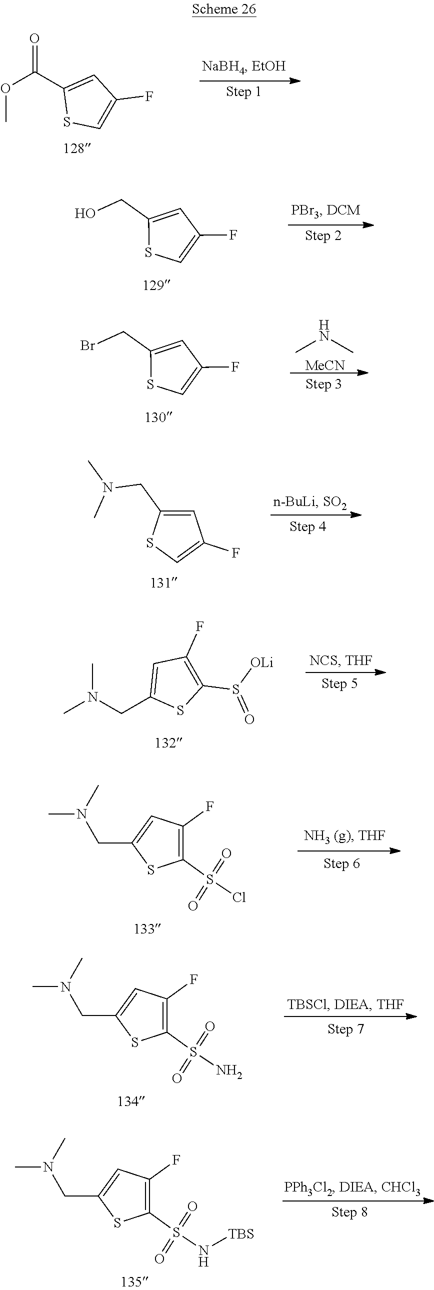

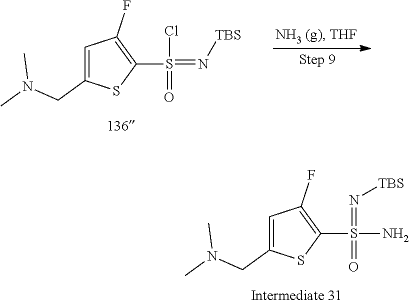

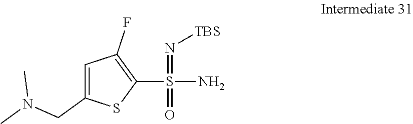

### N′-(tert-butyldimethylsilyl)-5-((dimethylamino)methyl)-3-fluorothiophene-2-sulfonimidamide

### Step 1: (4-Fluorothiophen-2-yl)methanol

Into a 1000-mL 3-necked round-bottom flask purged and maintained with an inert atmosphere of nitrogen, was placed methyl 4-fluorothiophene-2-carboxylate (10 g, 62.4 mmol) in EtOH (300 mL). To the above solution was added NaBH₄ (4.74 g, 124.8 mmol) with stirring at 0° C. The resulting solution was stirred for 30 min at 0° C. The resulting solution was allowed to react for an additional 16 h at RT. The reaction was then quenched by the addition of 10 mL of water. The resulting mixture was extracted with 3×1000 mL of ethyl acetate. Evaporation of combined ethyl acetate solution resulted in 6.4 g (77.5%) of the title compound as white oil.

### Step 2: 2-(Bromomethyl)-4-fluorothiophene

Into a 250-mL round-bottom flask, was placed (4-fluorothiophen-2-yl)methanol (8.5 g, 64.32 mmol) in DCM (70 mL). To the stirred solution was added PBr₃ (19.15 g, 70.75 mmol) at 0° C. The resulting solution was stirred for 30 min at 0° C., after which it was allowed to react for an additional 12 h at RT. The reaction was quenched with 20 mL of water and extracted with ethyl acetate 3×50 mL. The combined organic layer was dried over Na₂SO₄ and concentrated under vacuum. The residue was eluted from silica gel with ethyl acetate/petroleum ether (15/85). This resulted in 7.0 g (55.8%) of the title compound as yellow oil.

### Step 3: 1-(4-Fluorothiophen-2-yl)-N,N-dimethylmethanamine

Into a 250-mL round-bottom flask, was placed 2-(bromomethyl)-4-fluorothiophene (7.4 g, 37.9 mmol). To the solution was added dimethylamine in THF (2M, 37.9 mmol). The resulting solution was stirred for 16 h at RT. The reaction mixture was concentrated under vacuum. The residue was eluted from silica gel with ethyl acetate/petroleum ether (17/83). This resulted in 5.62 g (92.6%) of the title compound as a solid. MS-ESI: 160 [M+1].

### Step 4: Lithium 5-((dimethylamino)methyl)-3-fluorothiophene-2-sulfinate

Into a 250-mL 3-necked round-bottom flask purged and maintained with an inert atmosphere of nitrogen, was placed a solution of [(4-fluorothiophen-2-yl)methyl] dimethylamine (6.2 g, 38.9 mmol) in THF (60 mL), to the above solution was added n-BuLi (18.7 mL, 46.7 mmol, 2.5 M) dropwise at −78° C. in a liquid nitrogen/ethanol bath. The resulting solution was stirred for 30 min at −78° C. To the stirred solution, SO₂(g) (4.99 g, 78 mmol) was introduced in at −78° C. The resulting solution was allowed to react for an additional 120 min at RT. The resulting mixture was concentrated. This resulted in 10 g (crude) of the title compound as a yellow solid. MS-ESI: 228 [M−1].

### Step 5: 5-((Dimethylamino)methyl)-3-fluorothiophene-2-sulfonyl Chloride

Into a 500-mL round-bottom flask, was placed a solution of 5-[(dimethylamino)methyl]-3-fluorothiophene-2-sulfinic acid (10 g, 44.7 mmol) in THF (100 mL), to the above solution was added NCS (7.18 g, 54 mmol) at 0° C. The resulting solution was stirred for 30 min at 0° C. The resulting solution was allowed to react for an additional 100 min at RT. The reaction solution was used for next step without any purification.

### Step 6: 5-((Dimethylamino)methyl)-3-fluorothiophene-2-sulfonamide

Into a 500-mL round-bottom flask, was placed a solution of 5-[(dimethylamino)methyl]-3-fluorothiophene-2-sulfonyl chloride (10 g, 38.8 mmol) in THF (100 mL). To the above NH₃ (g) was introduced for 15 min at 0° C. The resulting solution was allowed to react for an additional 100 min at RT. Then the reaction solution was concentrated. The residue was eluted from silica gel with ethyl acetate/petroleum ether (60/40). This resulted in 2.1 g (22.7%) of the title compound as yellow oil. MS-ESI: 239 [M+1].

### Step 7: N-(tert-butyldimethylsilyl)-5-((dimethylamino)methyl)-3-fluorothiophene-2-sulfonamide

Into a 100-mL round-bottom flask purged and maintained with an inert atmosphere of nitrogen, was placed a solution of 5-[(dimethylamino)methyl]-3-fluorothiophene-2-sulfonamide (1.8 g, 7.55 mmol) in THF (30 mL). To the above solution was added NaH (60% wt. oil dispersion, 600 mg, 15 mmol) with stirring at 0° C. The resulting solution was stirred for 30 min at 0° C. This was followed by the addition of TBSCl (1.37 g, 9.09 mmol) at 0° C. The resulting solution was allowed to react for an additional 15 h at RT. The reaction solution was concentrated. The residue was eluted from silica gel with ethyl acetate. This resulted in 2 g (75.1%) of the title compound as yellow oil. MS-ESI: 353 [M+1].

### Step 8-1: N-(tert-butyldimethylsilyl)-5-((dimethylamino)methyl)-3-fluorothiophene-2-sulfonimidoyl Chloride

Into a 500-mL 3-necked round-bottom flask purged and maintained with an inert atmosphere of nitrogen, was placed a solution of PPh₃Cl₂ (59.2 g, 178 mmol) in CHCl₃ (100 mL). This was followed by the addition of DIEA (45.9 g, 355 mmol) dropwise with stirring at 0° C. The resulting solution was stirred for 15 min at RT. To this was added a solution of N-(tert-butyldimethylsilyl)-5-((dimethylamino)methyl)-3-fluorothiophene-2-sulfonamide (15.6 g, 44.4 mmol) in CHCl₃ (30 mL) dropwise with stirring at 0° C. The resulting solution was stirred for 30 min at 0° C. The reaction solution was used in the next step with no workup.

### Step 8-2: N′-(tert-butyldimethylsilyl)-5-((dimethylamino)methyl)-3-fluorothiophene-2-sulfonimidamide

Into a 250-mL 3-necked round-bottom flask, was placed N-(tert-butyldimethylsilyl)-5-((dimethylamino)methyl)-3-fluorothiophene-2-sulfonimidoyl chloride (2.8 g, 0.27 mmol) in CHCl₃ (20 mL). To the above NH₃(g) was introduced for 15 min at 0° C. The resulting solution was stirred for 15 min at 0° C. The resulting solution was stirred for 1 h at RT. The resulting mixture was diluted with 50 mL of H₂O. The resulting solution was extracted with 2×75 mL of DCM, the combined organic layer was dried over anhydrous sodium sulfate and concentrated. The residue was eluted from silica gel with ethyl acetate/petroleum ether (1:6). This resulted in 250 mg (9.4%) of the title compound as a yellow solid. MS-ESI: 352 [M+1].

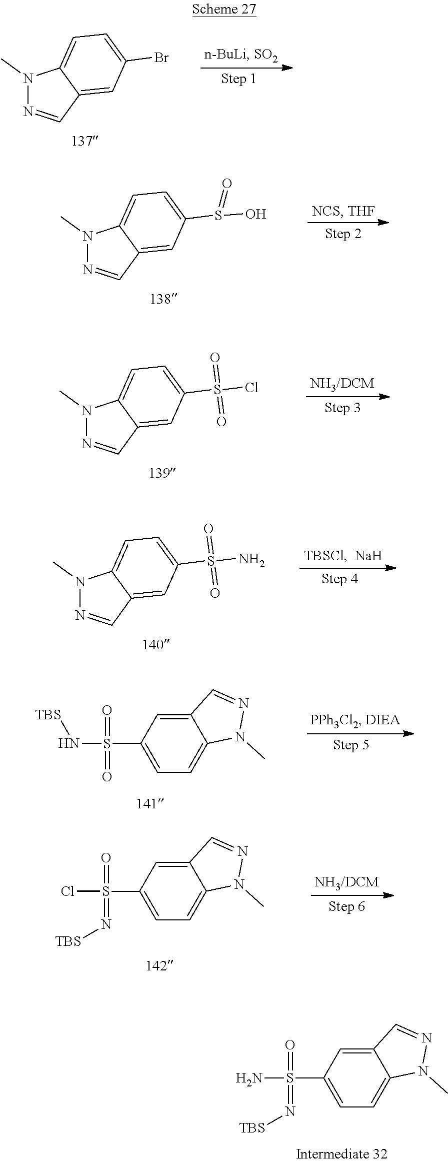

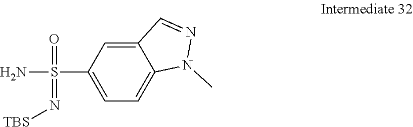

### Step 1: 1-Methyl-1H-indazole-5-sulfinic Acid

Into a 100-mL 3-necked round-bottom flask purged and maintained with an inert atmosphere of nitrogen, was placed 5-bromo-1-methyl-1H-indazole (700 mg, 3.32 mmol) in THF (5 mL). To the above solution was added n-BuLi (1.6 mL, 3.98 mmol, 2.5 M) dropwise at −78° C. in a liquid nitrogen/ethanol bath. Then the solution was stirred for 30 min at −78° C. To the stirred solution, SO₂ (g) was introduced at −78° C. for 15 min. The resulting solution was allowed to react for an additional 120 min at RT. The resulting mixture was concentrated. This resulted 500 mg (76.8%) of the title compound as a yellow solid.

Steps 2-6 used similar procedures for converting compound 132″ to Intermediate 31 shown in Scheme 26 to afford Intermediate 32 from compound 138″. MS-ESI. 325 (M+1).

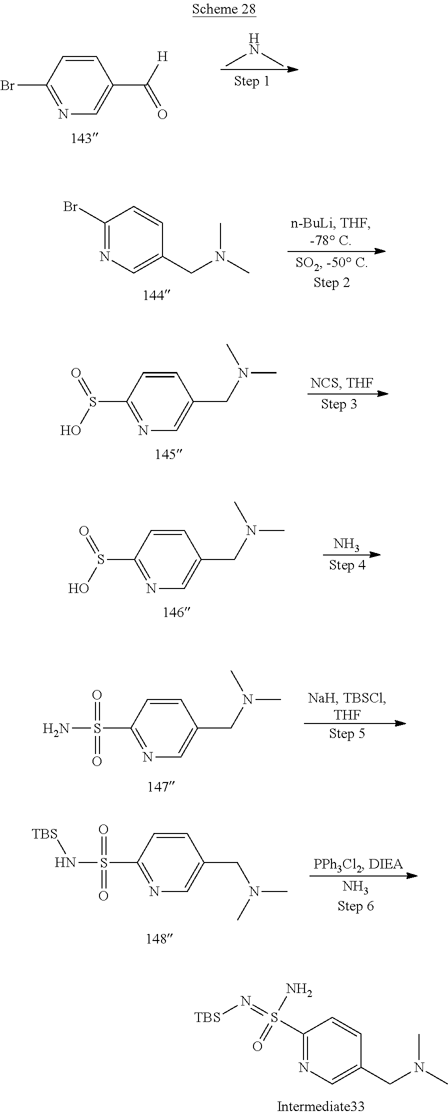

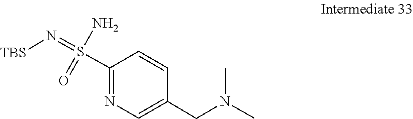

### N′-(tert-butyldimethylsilyl)-5-((dimethylamino)methyl)pyridine-2-sulfonimidamide

### Step 1: 1-(6-Bromopyridin-3-yl)-N,N-dimethylmethanamine

Into a 500 mL round-bottom flask, were added Ti(OEt)₄ (12.2 g, 53.7 mmol) and dimethylamine (4.85 g, 107 mmol) in methanol (50 mL) at RT. To this stirred solution was added 6-bromopyridine-3-carbaldehyde (5 g, 26.9 mmol) in methanol (30 mL) dropwise at 0° C. After stirring at RT for 3 h, NaBH₄ (1.02 g, 26.9 mmol) was added and the resulting mixture was stirred overnight. The reaction was quenched by the addition of water/ice (30 mL) at 0° C. The resulting mixture was extracted with ethyl acetate (3×50 mL). The combined organic layers were dried over anhydrous Na₂SO₄. After filtration, the filtrate was concentrated under reduced pressure. The residue was purified by silica gel chromatography, eluted with ethyl acetate/petroleum ether (5:1) to afford the title compound (3.5 g, 60.5%) as yellow oil. MS-ESI: 215 (M+1).

### Step 2: 5-((Dimethylamino)methyl)pyridine-2-sulfinic Acid

Into a 250-mL 3-necked round-bottom flask purged and maintained with an inert atmosphere of nitrogen, was placed [(6-bromopyridin-3-yl)methyl]dimethylamine (3.5 g, 16.27 mmol) in THF (30 mL). This was followed by the addition of n-BuLi (7.2 mL, 17.9 mmol, 2.5 M) dropwise with stirring at −78° C. in 30 min. To this was bubbled SO₂ at −78° C. for 15 min. The resulting solution was stirred for 1 h at −78° C. The resulting mixture was concentrated under vacuum. The crude product the title compound (4.0 g) was used in the next step directly without further purification.

### Step 3: 5-((Dimethylamino)methyl)pyridine-2-sulfonyl Chloride

Into a 250 mL round-bottom flask, was placed 5-[(dimethylamino)methyl]pyridine-2-sulfinic acid (4.0 g crude) and THF (25 mL) at RT. To a stirred solution was added NCS (4 g, 0.03 mmol) in portions at 0° C. The resulting solution was stirred for 1.5 h at RT. The resulting mixture was used in the next step with no workup.

### Step 4: 5-((Dimethylamino)methyl)pyridine-2-sulfonamide

Into a 250 mL round-bottom flask, were added 5-[(dimethylamino)methyl]pyridine-2-sulfonyl chloride (crude from previous step) at RT. To this was bubbled NH₃ (g) for 10 min at 0° C. The resulting mixture was stirred for 1 h at 0° C. The residue was purified by reverse-phase flash chromatography with the following conditions (column, Cis silica gel; mobile phase, MeCN in water, 10% to 50% gradient in 10 min; detector, UV 254 nm.) to afford the title compound (1.2 g, 32.7%) as a yellow solid. MS-ESI: 216 [M+1]

### Step 5: N-(tert-butyldimethylsilyl)-5-((dimethylamino)methyl)pyridine-2-sulfonamide

Into a 100 mL round-bottom flask, were added 5-[(dimethylamino)methyl]pyridine-2-sulfonamide (700 mg, 3.25 mmol) in THF (15 mL) at 0° C. To this stirred solution was added NaH (60% wt. oil dispersion, 260 mg, 6.5 mmol) in portions at 0° C. under nitrogen atmosphere. The resulting mixture was stirred for 15 min at 0° C. under nitrogen atmosphere. Then TBSCl (980 mg, 6.5 mmol) was added to the above reaction mixture. After the addition was complete, the resulting mixture was stirred for 2 h at RT. The reaction was quenched by the addition of water/ice (10 mL) at 0° C. The resulting mixture was extracted with ethyl acetate (3×20 mL). The combined organic layers were dried over anhydrous Na₂SO₄. After filtration, the filtrate was concentrated under reduced pressure. The crude title compound (1.15 g) was used in the next step directly without further purification. MS-ESI: 330 [M+1].

### Step 6: N′-(tert-butyldimethylsilyl)-5-((dimethylamino)methyl)pyridine-2-sulfonimidamide

Into a 250 mL 3-necked round-bottom flask, was added PPh₃Cl₂ (5.89 g, 13.9 mmol) in CHCl₃ (18 mL). To this stirred solution was added DIEA (3.61 g, 27.9 mmol) dropwise at 0° C. under nitrogen atmosphere. The resulting mixture was stirred for 15 min at 0° C. under nitrogen atmosphere. Then N-(tert-butyldimethylsilyl)-5-[(dimethylamino)methyl]pyridine-2-sulfonamide (1.15 g, 3.49 mmol) in CHCl₃ (3 mL) was added to the above resulting mixture dropwise at 0° C. under nitrogen atmosphere. After the addition was complete the resulting mixture was stirred for 30 min. Then NH₃ (g) in DCM (40 mL) was added to the resulting mixture. The resulting mixture was stirred overnight. The resulting mixture was filtered, the filter cake was washed with ethyl acetate (3×20 mL). The filtrate was concentrated under reduced pressure. The residue was purified by Prep-TLC (ethyl acetate/methanol 100:1) to afford the title compound (600 mg, 52.3%) as a yellow solid. MS-ESI: 329 (M+1).

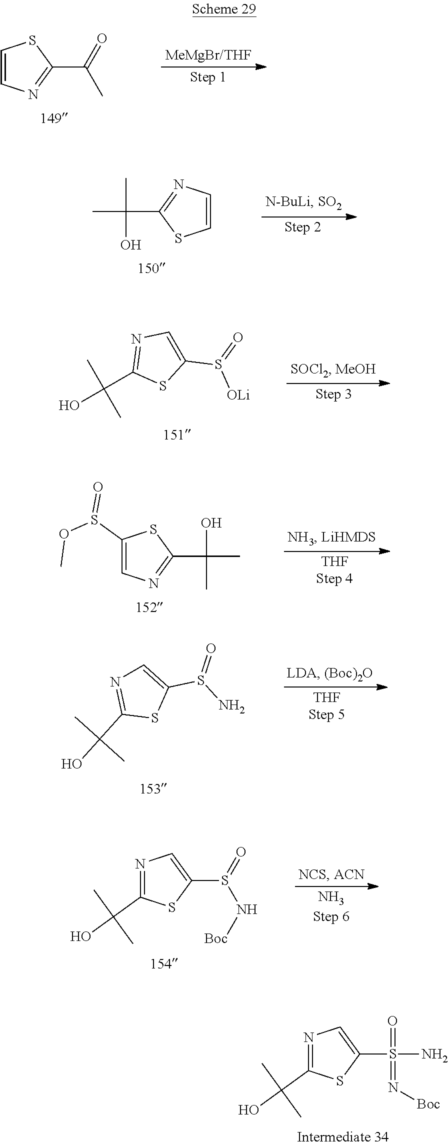

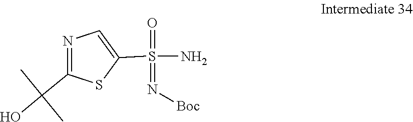 [SMILES: CC(C)(C)OC(=O)N=S(N)(=O)c1cnc(C(C)(C)O)s1]

### N′-(tert-butoxycarbonyl)-2-(2-hydroxypropan-2-yl)thiazole-5-sulfonimidamide

### Step 1: 2-(Thiazol-2-yl)propan-2-ol

Into a 10-L 4-necked round-bottom flask purged with and maintained under nitrogen was placed a solution of 1-(thiazol-2-yl)ethanone (200 g, 1.6 mol) in THF (4 L). This was followed by the addition of MeMgBr (3 M in THF, 942 mL) dropwise with stirring at 0° C. The mixture was stirred at 0° C. for 2 h. After warmed the mixture to RT, the solution was stirred for an additional 16 h. Then the reaction was quenched by the addition of 3 L of NH₄Cl (sat.). The resulting solution was extracted with 3×1 L of ethyl acetate. The organic layers were combined and dried over anhydrous Na₂SO₄, then concentrated under vacuum. The residue was applied onto a silica gel column and eluted with a gradient of ethyl acetate/petroleum ether (1:3 to 1:1). This resulted in 210 g (93%) of the title compound as brown oil. MS-ESI: 144.0 (M+1). ¹H NMR (400 MHz, DMSO-d₆) δ 7.68 (d, J=3.2 Hz, 1H), 7.54 (d, J=3.2 Hz, 1H), 5.94 (s, 1H), 1.51 (s, 6H).

### Step 2: Lithium 2-(2-hydroxypropan-2-yl)thiazole-5-sulfinate

Into a 10-L 4-necked round-bottom flask purged with and maintained under nitrogen, was placed a solution of 2-(thiazol-2-yl)propan-2-ol (50 g, 349 mmol) in THF (1.5 L). This was followed by the addition of n-BuLi (2.5 M in hexane, 350 mL) dropwise with stirring at −78° C. The mixture was stirred at −78° C. for 1 h. Then SO₂ was bubbled into the mixture for 15 min below −30° C. The mixture was stirred for an additional 1 h at RT and then was concentrated under vacuum. This resulted in 87 g (crude) of the title compound as a light yellow solid. The crude product was used directly in the next step.

### Step 3: Methyl 2-(2-hydroxypropan-2-yl)thiazole-5-sulfinate

Into a 2-L 3-necked round-bottom flask, lithium 2-(2-hydroxypropan-2-yl)thiazole-5-sulfinate (87 g, crude) was dissolved in anhydrous MeOH (500 mL). Then SOCl₂ (43 g, 360 mmol) was added to the mixture dropwise with stirring at 0° C. The mixture was stirred overnight at RT and then was concentrated under vacuum. The residue was diluted with 500 mL of ethyl acetate. The resulting solution was washed with 2×200 mL of water and 2×200 mL of brine. The organic phase was dried over anhydrous Na₂SO₄, then concentrated under vacuum. This resulted in 72 g (crude) title compound as light yellow oil. The crude product was used directly in the next step. MS-ESI: 222[M+1]. ¹H NMR (400 MHz, DMSO-d₆) δ 8.15 (s, 1H), 6.32 (s, 1H), 3.65 (s, 3H), 1.53 (d, J=2.0 Hz, 6H).

### Step 4: 2-(2-Hydroxypropan-2-yl)thiazole-5-sulfinamide

Into a 10-L 4-necked round-bottom flask purged with and maintained under nitrogen, was placed a solution of methyl 2-(2-hydroxypropan-2-yl)thiazole-5-sulfinate (72 g, 326 mmol) in THF (500 mL). Then to the above NH₃ (0.5 M in THF, 2.0 L) was added. After cooling to −78° C., LiHMDS (1 M in THF, 2.0 L) was added to the mixture dropwise with stirring. Then the mixture was stirred at −78° C. for 2 h. The reaction was quenched by the addition of 500 mL of NH₄Cl (sat.). The resulting solution was extracted with 3×300 mL of ethyl acetate. The organic layers were combined, dried over anhydrous Na₂SO₄, then concentrated under vacuum. This resulted in 32 g (crude) title compound as brown oil. The crude product was used directly in the next step. MS-ESI: 207 [M+1]. ¹H NMR (400 MHz, DMSO-d₆) δ 7.77 (s, 1H), 6.73 (s, 2H), 6.17 (s, 1H), 1.51 (d, J=1.4 Hz, 6H).

### Step 5: Tert-butyl 2-(2-hydroxypropan-2-yl)thiazol-5-ylsulfinylcarbamate

Into a 1-L 3-necked round-bottom flask purged with and maintained under nitrogen, was placed a solution of 2-(2-hydroxypropan-2-yl)thiazole-5-sulfinamide (32 g, crude) in THF (300 mL). This was followed by the addition of LDA (2 M in THF, 116 mL) dropwise with string at 0° C. The mixture was stirred at 0° C. for 1 h, then (Boc)₂O (33.8 g, 155 mmol) was added in portions at 0° C. The mixture was warmed to RT and stirred for an additional 2 h. The reaction was quenched with 200 mL of ice-water (200 mL), and the pH value of the solution was adjusted to 6 with HCOOH. The resulting solution was extracted with 3×200 mL of ethyl acetate. The organic layers were combined, dried over anhydrous Na₂SO₄, and then concentrated under vacuum. The residue was eluted from silica gel with a gradient of ethyl acetate/petroleum ether (1:2 to 1:1). This resulted in 19 g (18%, 4 steps) title compound as a white solid. MS-ESI: 307 [M+1].

### Step 6: N-(tert-butyldimethylsilyl)-2-(2-hydroxypropan-2-yl)thiazole-5-sulfonimidamide

Into a 1-L 3-necked round-bottom flask purged with and maintained under nitrogen, tert-butyl 2-(2-hydroxypropan-2-yl)thiazol-5-ylsulfinylcarbamate (19 g, 62 mmol) was dissolved in freshly distilled ACN (200 mL). Then to the above solution was added NCS (9.8 g, 74 mmol) in portions. The mixture was stirred for 1 h at RT and then NH₃ was bubbled in the mixture for 15 min. The mixture was stirred at RT for 2 h and then was concentrated under vacuum. The residue was applied onto a silica gel column and eluted with a gradient of ethyl acetate/petroleum ether (1:2 to 1:1). This resulted in 13 g (65%) of the title compound as a white solid. MS-ESI: 322 [M+1]. ¹H NMR (300 MHz, DMSO-d₆) δ 7.99 (s, 1H), 7.72 (s, 2H), 6.29 (s, 1H), 1.49 (d, J=2.0 Hz, 6H), 1.27 (s, 9H).

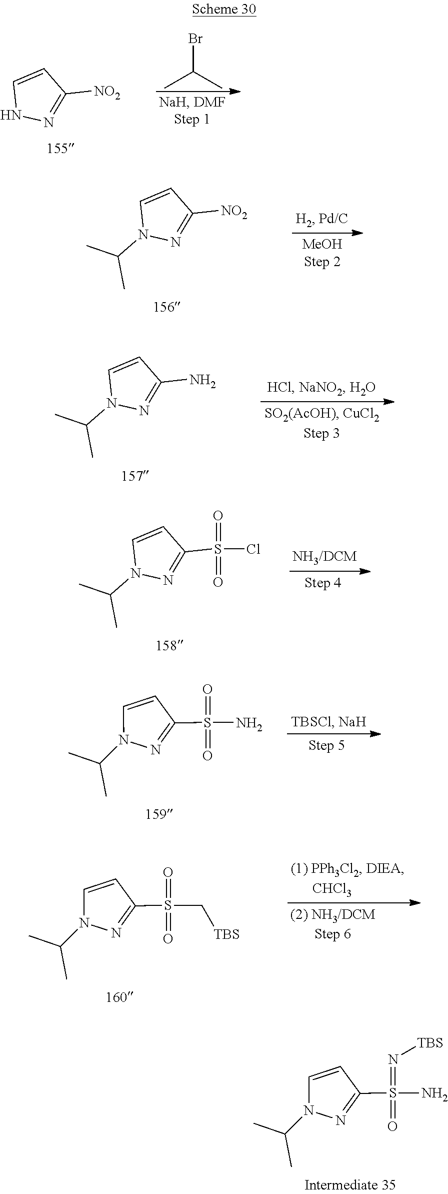

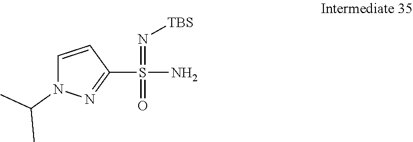

### N′-(tert-butyldimethylsilyl)-1-isopropyl-1H-pyrazole-3-sulfonimidamide

### Step 1: 1-Isopropyl-3-nitro-1H-pyrazole

Into a 250-mL round-bottom flask, was placed a solution of 3-nitro-1H-pyrazole (10 g, 88.4 mmol) in DMF (100 mL). This was followed by the addition of NaH (60% wt. oil dispersion, 3.9 g, 97.5 mmol) in portions at 0° C. The resulting solution was stirred for 0.5 h at 0° C. This was followed by the addition of 2-bromopropane (14.1 g, 114.6 mmol) dropwise with stirring at 0° C. in 10 min. The resulting solution was stirred for 16 h at RT and then was quenched by the addition of 100 mL of water. The resulting solution was extracted with 3×100 mL of ethyl acetate. The organic layers were combined and dried over anhydrous Na₂SO₄, and then concentrated under vacuum. The residue was eluted from silica gel and eluted with a gradient of ethyl acetate/petroleum ether (1:5 to 1:3). This resulted in 11.8 g (86%) of the title compound as yellow oil. MS-ESI: 156.1 (M+1).

### Step 2: 3-Amino-1-(propan-2-yl)-1H-pyrazole

Into a 250-mL round-bottom flask, was placed a solution of 1-isopropyl-3-nitro-1H-pyrazole (10.8 g, 69.6 mmol) in MeOH (100 mL). Then Pd/C (10% wt., 1.5 g) was added. The flask was evacuated and flushed three times with hydrogen. The mixture was stirred for 24 h at RT under an atmosphere of hydrogen. The solids were filtered out. The resulting filtrate was concentrated under vacuum. This resulted in 7.27 g (83%) of the title compound as yellow oil. MS-ESI: 126.1 (M+1).

### Step 3: 1-isopropyl-1H-pyrazole-3-sulfonyl Chloride

Into a 1 L round-bottom flask, was placed a solution of 3-amino-1-(propan-2-yl)-1H-pyrazole (10 g, 80 mmol) in aq. HCl (6 N, 200 mL). This was followed by the addition of a solution of NaNO₂ (8.28 g, 120 mmol) in water (20 mL) dropwise with stirring at 0° C. The resulting solution was stirred for 30 min at 0° C. The above mixture was added to a saturated solution of SO₂ in AcOH (200 mL) dropwise with stirring at 0° C. Then to the above was added CuCl₂ (10.8 g, 80.7 mmol). The resulting solution was stirred for 1 h at RT and was then quenched by the addition of 200 mL of water. The resulting solution was extracted with 3×200 mL of DCM. The organic layers were combined, dried over anhydrous Na₂SO₄ and concentrated under vacuum. This resulted in 10 g (59.8%) of the title compound as yellow oil. The product was used in the next step without further purification.

### Step 4: 1-isopropyl-1H-pyrazole-3-sulfonamide

Into a 1000 mL round bottom flask, was placed a solution of 1-isopropyl-1H-pyrazole-3-sulfonyl chloride (10 g, 47.8 mmol) in DCM (50 mL). This was followed by the addition of a saturated solution of ammonia in DCM (500 mL) in portions with stirring at 0° C. The resulting solution was stirred for 1 h at 0° C. The resulting solution was concentrated and the residue was purified with SiO₂-gel column and eluted with ethyl acetate/petroleum ether (1:2 to 1:1). This resulted in 8.13 g (90%) of the title compound as yellow solid. MS-ESI: 190 [M+1].

Steps 5-6 used similar procedures for converting compound 147″ to Intermediate 33 shown in Scheme 28 to afford compound intermediate 35 from compound 159″. MS-ESI: 303 (M+1).

Schemes for phenylacetic acids Intermediates: Schemes 31-47 illustrate the preparation of phenylacetic acid intermediates.

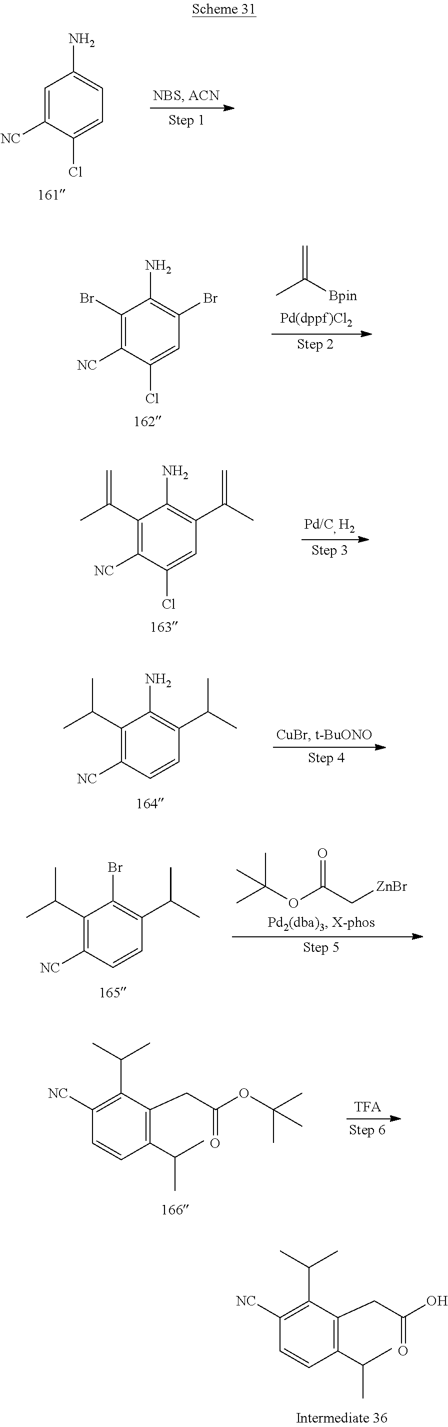

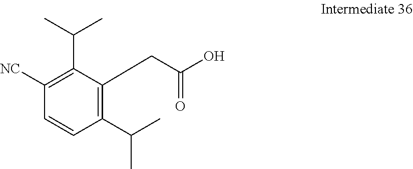 [SMILES: CC(C)c1ccc(C#N)c(C(C)C)c1CC(=O)O]

### 2-(3-Cyano-2,6-diisopropylphenyl)acetic Acid

### Step 1: 3-Amino-2,4-dibromo-6-chlorobenzonitrile

Into a 500-mL round-bottom flask, was placed 5-amino-2-chlorobenzonitrile (10 g, 65.7 mmol) in ACN (200 mL). To the stirred solution was added NBS (29 g, 162 mmol) in portions. The resulting solution was stirred for 14 h at RT. The resulting mixture was concentrated. The residue was eluted from silica gel with ethyl acetate/petroleum ether (1:15 to 1:5). This resulted in 18 g of the title compound as a yellow solid. MS-ESI: 308/310 (M+1).

### Step 2: 3-Amino-6-chloro-2,4-di(prop-1-en-2-yl)benzonitrile

Into a 500-mL round-bottom flask purged and maintained with an inert atmosphere of nitrogen, was placed 3-amino-2,4-dibromo-6-chlorobenzonitrile (15 g, 48.0 mmol) in dioxane (200 mL) and H₂O (20 mL), 2-(tetramethyl-1,3,2-dioxaborolan-2-yl)prop-2-en-1-ylium (18.5 g, 111 mmol), Cs₂CO₃ (47 g, 144 mmol) and Pd(dppf)Cl₂ (1.5 g). The resulting solution was stirred for 14 h at 100° C. in an oil bath. The resulting mixture was concentrated. The residue was eluted from silica gel with ethyl acetate/petroleum ether (1:0 to 1:25). This resulted in 10 g of the title compound as brown oil. MS-ESI: 233 (M+1).

### Step 3: 3-Amino-2,4-diisopropylbenzonitrile

Into a 500-mL round-bottom flask purged and maintained with an inert atmosphere of nitrogen, was placed 3-amino-6-chloro-2,4-bis(prop-1-en-2-yl)benzonitrile (10 g, 43 mmol) in methanol (50 mL), to the stirred solution was added Pd/C (10% wt., 2 g). The flask was evacuated and filled three times with hydrogen. The resulting solution was stirred overnight at RT. The solids were filtered out. The resulting mixture was concentrated under vacuum. This resulted in 8 g of the title compound as brown oil. MS-ESI: 203 (M+1).

### Step 4: 3-Bromo-2,4-diisopropylbenzonitrile

Into a 250-mL round-bottom flask, was placed 3-amino-2,4-bis(propan-2-yl)benzonitrile (8 g, 39.5 mmol) in ACN (150 mL), to the stirred solution was added CuBr (11.3 g, 79.1 mmol) and tert-butyl nitrite (8.2 g, 79.1 mmol). The resulting solution was stirred for 3 h at 60° C. in an oil bath. The resulting mixture was concentrated. The residue was eluted from silica gel with ethyl acetate/petroleum ether (1:50). This resulted in 4.2 g (39.90%) of the title compound as purple oil. MS-ESI: 266/268[M+1]

### Step 5: Tert-butyl 2-(3-cyano-2,6-diisopropylphenyl)acetate

Into a 250-mL round-bottom flask purged and maintained with an inert atmosphere of nitrogen, was placed 3-bromo-2,4-bis(propan-2-yl)benzonitrile (3.1 g, 11.6 mmol) in THF (100 mL), to the stirred solution was added Xphos (555.2 mg, 1.16 mmol), Pd₂(dba)₃ (533.2 mg, 0.58 mmol) and tert-butyl 2-(bromozincio)acetate (7.6 g, 29.12 mmol). The resulting solution was stirred for 3 h at 65° C. in an oil bath. The resulting mixture was concentrated. The residue was eluted from silica gel with ethyl acetate/petroleum ether (1:50). This resulted in 3.0 g (85.5%) of the title compound as purple oil. MS-ESI: 302 [M+1].

### Step 6: 2-(3-Cyano-2,6-diisopropylphenyl)acetic Acid

Into a 100-mL round-bottom flask, was placed tert-butyl 2-[3-cyano-2,6-bis(propan-2-yl)phenyl]acetate (3.4 g, 11.28 mmol) in DCM (15 mL), to the stirred solution was added TFA (15 mL). The resulting solution was stirred for 3 h at RT. The resulting mixture was concentrated. The residue was eluted from silica gel with ethyl acetate/petroleum ether (1:3). This resulted in 2.6 g (93.9%) of the title compound as a light yellow solid. MS-ESI: 246 [M+1].

TABLE 13

| Intermediate | | | |
|---|---|---|---|
| # | Structure | IUPAC Name | Exact Mass[M − H]⁻ |
| Intermediate 37 | 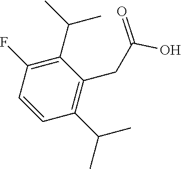 [SMILES: CC(C)c1ccc(F)c(C(C)C)c1CC(=O)O] | 2-(3-fluoro-2,6-diisopropylphenyl)acetic acid | 237 |
| Intermediate 38 | 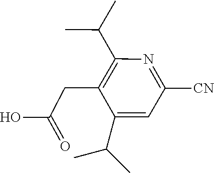 [SMILES: CC(C)c1cc(C#N)nc(C(C)C)c1CC(=O)O] | 2-(6-cyano-2,4-diisopropylpyridin-3-yl)acetic acid | 247 |

The Intermediates in the following Table were prepared using the similar procedures for converting compound 161″ to Intermediate 36 shown in Scheme 31 from appropriated starting materials.

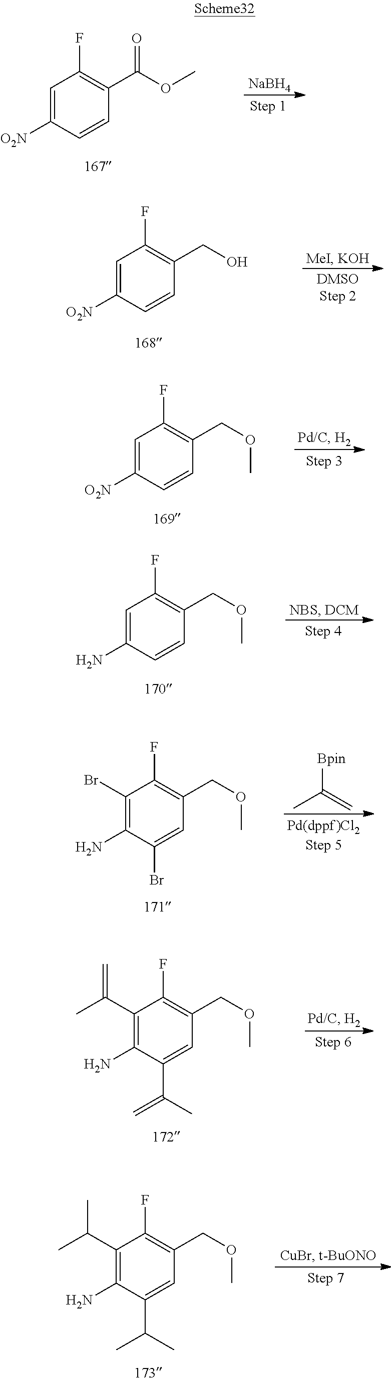

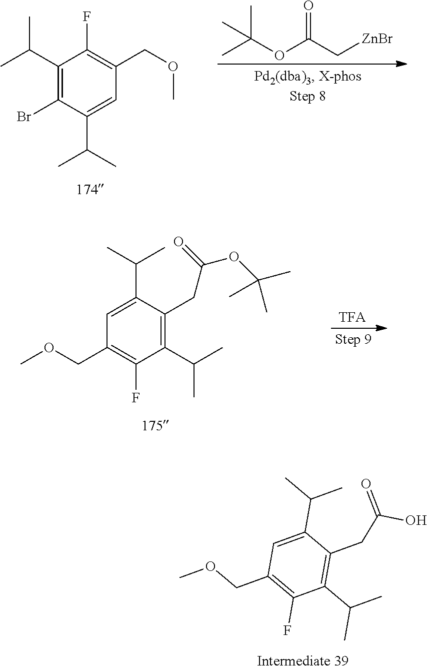

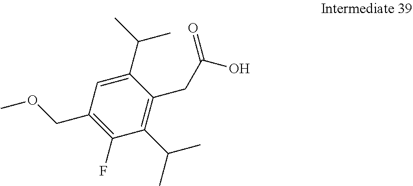 [SMILES: COCc1cc(C(C)C)c(CC(=O)O)c(C(C)C)c1F]

### 2-(3-Fluoro-2,6-diisopropyl-4-(methoxymethyl)phenyl)acetic Acid

### Step 1: (2-Fluoro-4-nitrophenyl)methanol

Into a 500-mL round-bottom flask, was placed methyl 2-fluoro-4-nitrobenzoate (10 g, 50.2 mmol) in methanol (100 mL). This was followed by the addition of NaBH₄ (9.5 g, 251 mmol) in portions over 30 min. The resulting solution was stirred for 4 h at RT. The resulting solution was diluted with 400 mL of ethyl acetate. The resulting mixture was washed with 200 mL of water and 200 mL of brine. The organic layer was dried over anhydrous sodium sulfate and then concentrated. This resulted in 3.6 g of the title compound as an off white solid. MS-ESI: 172 (M+1).

### Step 2: 2-Fluoro-1-(methoxymethyl)-4-nitrobenzene

Into a 50-mL round-bottom flask, was placed (2-fluoro-4-nitrophenyl)methanol (3.6 g, 21.0 mmol) in DMSO (10 mL). To the stirred solution was added KOH (4.72 g, 84.2 mmol) in portions and MeI (11.9 g, 84.1 mmol) dropwise at RT. The resulting solution was stirred for overnight at RT. The reaction was then quenched by the addition of water. The resulting solution was extracted with 200 mL of dichloromethane. The organic layers were combined and washed with 200 mL of brine. Then the organic layer was dried over anhydrous sodium sulfate and concentrated. The residue was eluted from silica gel with ethyl acetate/petroleum ether (1:1) to give the title compound as 2.1 g yellow solid. MS-ESI: 186 (M+1).

### Step 3: 3-Fluoro-4-(methoxymethyl)aniline

Into a 250-mL round-bottom flask purged and maintained with an inert atmosphere of nitrogen, was placed 2-fluoro-1-(methoxymethyl)-4-nitrobenzene (2.4 g, 12.9 mmol) in methanol (50 mL), to the stirred solution was added Pd/C (10% wt. oil dispersion, 240 mg). The flask was evacuated and filled three times with hydrogen. The resulting solution was stirred overnight at RT. The solids were filtered out. The resulting mixture was concentrated under vacuum. The resulting mixture was concentrated to give the title compound as 2.4 g yellow solid. MS-ESI: 156 (M+1).

### Step 4: 2,6-Dibromo-3-fluoro-4-(methoxymethyl)aniline

Into a 100-mL round-bottom flask, was placed 3-fluoro-4-(methoxymethyl)aniline (1.7 g, 10.96 mmol) in DCM (50 mL). This was followed by the addition of NBS (4.3 g, 12.1 mmol) in portions. The resulting solution was stirred for 1 h at RT. The reaction was then quenched by the addition of water/ice. The resulting solution was extracted with 200 mL of ethyl acetate. The resulting mixture was washed with 200 mL of water and 200 mL of brine. The organic layer was dried over anhydrous sodium sulfate and concentrated. The residue was eluted from silica gel with ethyl acetate/petroleum ether (1:3) to give 4 g title compound as a yellow solid. MS-ESI: 311/313 (M+1).

### Step 5: 3-Fluoro-4-(methoxymethyl)-2,6-di(prop-1-en-2-yl)aniline

Into a 500-mL round-bottom flask purged and maintained with an inert atmosphere of nitrogen, was placed 2,6-dibromo-3-fluoro-4-(methoxymethyl)aniline (14 g, 44.7 mmol) in dioxane (200 mL) and H₂O (20 mL). To the stirred solution was added 4,4, 5, 5-tetramethyl-2-(prop-1-en-2-yl)-1,3,2-dioxaborolane (18.8 g, 111 mmol), Pd(dppf)Cl₂ (3.27 g, 4.47 mmol) and Cs₂CO₃ (29.2 g, 89.5 mmol). The resulting solution was stirred for 5 h at 65° C. in an oil bath. The reaction was then quenched by the addition of 10 mL of water. The resulting solution was extracted with 3×20 ml of ethyl acetate and dried over anhydrous sodium sulfate and concentrated. The residue was eluted from silica gel with ethyl acetate/petroleum ether (1:5). This resulted in 2.0 g (19.0%) of the title compound as yellow oil. MS-ESI: 236 (M+1).

### Step 6: 3-Fluoro-2,6-diisopropyl-4-(methoxymethyl)aniline

Into a 100-mL round-bottom flask, was placed 3-fluoro-4-(methoxymethyl)-2,6-bis(prop-1-en-2-yl) aniline (2.0 g, 8.50 mmol) in methanol (20 mL). To the stirred solution was added Pd/C (10% wt., 200 mg). The flask was evacuated and filled three times with hydrogen. The resulting solution was stirred 5 h at RT. The solids were filtered out. The resulting filtrate was concentrated under vacuum. This resulted in 1.8 g (88.5%) of the title compound as yellow oil. MS-ESI: 240 (M+1).

### Step 7: 2-Bromo-4-fluoro-1,3-diisopropyl-5-(methoxymethyl)benzene

Into a 100-mL round-bottom flask purged and maintained with an inert atmosphere of nitrogen, was placed 3-fluoro-4-(methoxymethyl)-2,6-bis(propan-2-yl)aniline (1.0 g, 4.18 mmol) in CH₃CN (30 mL). To the above solution was added CuBr (2.4 g, 16.7 mmol) and t-BuONO (−1.72 g, 16.7 mmol) with stirring. The resulting solution was stirred for 3 h at 65° C. in an oil bath. The reaction was then quenched by the addition of 20 mL of water. The resulting solution was extracted with 3×20 ml of ethyl acetate and dried over anhydrous sodium sulfate and concentrated. The residue was eluted from silica gel with petroleum ether. This resulted in 500 mg (39.4%) of the title compound as a yellow solid. MS-ESI: 303/305 [M+1].

### Step 8: Tert-butyl 2-(3-fluoro-2,6-diisopropyl-4-(methoxymethyl)phenyl)acetate

Into a 100-mL round-bottom flask purged and maintained with an inert atmosphere of argon, was placed 2-bromo-4-fluoro-5-(methoxymethyl)-1,3-bis(propan-2-yl)benzene (1.0 g, 3.30 mmol) in THF (40 mL). To the stirred solution was added tert-butyl 2-(bromozincio)acetate (2.58 g, 9.89 mmol), Pd₂(dba)₃CHCl₃ (170 mg, 0.16 mmol), and Xphos (157 mg, 0.33 mmol). The resulting solution was stirred for 3 h at 65° C. in an oil bath. The reaction was then quenched by the addition of 20 mL of water. The resulting solution was extracted with 3×30 ml of DCM and dried over anhydrous sodium sulfate and concentrated. The residue was eluted from silica gel with ethyl acetate/petroleum ether (1:20). This resulted in 200 mg (17.9%) of the title compound as yellow oil. MS-ESI: 339 [M+1].

### Step 9: 2-(3-Fluoro-2,6-diisopropyl-4-(methoxymethyl)phenyl)acetic Acid

Into a 50-mL round-bottom flask, was placed tert-butyl 3-fluoro-4-(methoxymethyl)-2,6-bis(propan-2-yl)benzoate (300 mg, 0.92 mmol) in DCM (6 mL), to the stirred solution was added TFA (2 mL). The resulting solution was stirred for 2 h at RT. The resulting mixture was concentrated. The residue was eluted from silica gel with DCM/methanol (1:20). This resulted in 170 mg of the title compound as yellow oil. MS-ESI: 281 (M−1).

TABLE 14

| Intermediate | | | Exact Mass |
|---|---|---|---|
| # | Structure | IUPAC Name | [M − H]⁻ |
| Intermediate 40 | 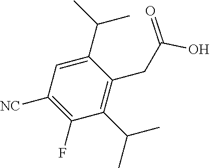 [SMILES: CC(C)c1cc(C#N)c(F)c(C(C)C)c1CC(=O)O] | 2-(4-cyano-3-fluoro-2,6-diisopropylphenyl)acetic acid | 262 |
| Intermediate 41 | 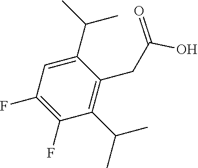 [SMILES: CC(C)c1cc(F)c(F)c(C(C)C)c1CC(=O)O] | 2-(3,4-difluoro-2,6-diisopropylphenyl)acetic acid | 255 |
| Intermediate 42 | 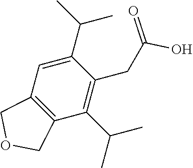 [SMILES: CC(C)c1cc2c(c(C(C)C)c1CC(=O)O)COC2] | 2-(4,6-diisopropyl-1,3-dihydroisobenzofuran-5-yl)acetic acid | 261 |
| Intermediate 43 | 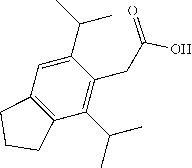 [SMILES: CC(C)c1cc2c(c(C(C)C)c1CC(=O)O)CCC2] | 2-(4,6-diisopropyl-2,3-dihydro-1H-inden-5-yl)acetic acid | 259 |

The Intermediates in the following Table were prepared using the similar procedures for converting compound 167″ to Intermediate 39 shown in Scheme 32 from appropriated starting materials.

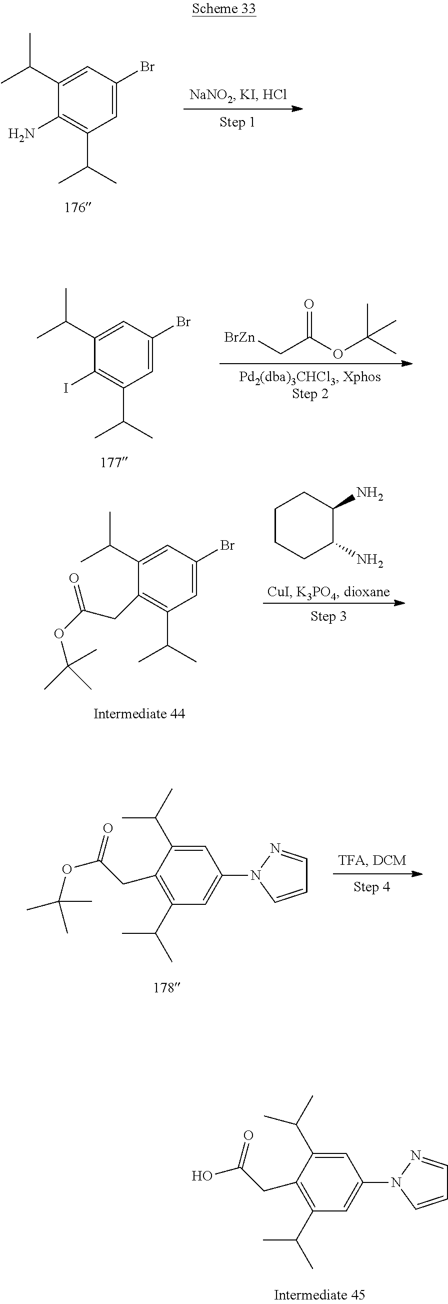

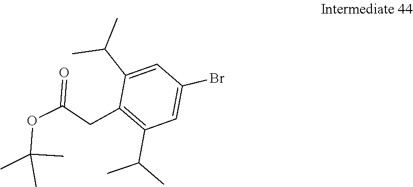 [SMILES: CC(C)c1cc(Br)cc(C(C)C)c1CC(=O)OC(C)(C)C]

### Tert-butyl 2-(4-bromo-2,6-diisopropylphenyl)acetate

### Step 1: 5-Bromo-2-iodo-1,3-diisopropylbenzene

Into a 500-mL round-bottom flask purged and maintained with an inert atmosphere of nitrogen, was placed 4-bromo-2,6-bis(propan-2-yl)aniline (5 g, 19.6 mmol) in HCl (6 M, 60 mL). This was followed by the addition of a solution of NaNO₂ (2.5 g, 36.3 mmol) in water (5 mL) dropwise with stirring at −10° C. The resulting solution was stirred for 30 min at −10° C. Then to the above was added KI (11 g, 66.3 mmol). The resulting solution was stirred for 1 h at RT. The reaction was then quenched by the addition of 100 mL of water. The resulting solution was extracted with 2×200 mL of ethyl acetate and the organic layers combined. The residue was eluted from silica gel with ethyl acetate/petroleum ether (0/1). This resulted in 5.95 g (83.0%) of the title compound as a brown liquid. MS-ESI: 366/368 (M+1).

### Step 2: Tert-butyl 2-(4-bromo-2,6-diisopropylphenyl)acetate

Into a 100-mL round-bottom flask purged and maintained with an inert atmosphere of nitrogen, was placed 5-bromo-2-iodo-1,3-bis(propan-2-yl)benzene (2.0 g, 5.45 mmol) in THF (50 mL). To the stirred solution was added Pd₂(dba)₃ (504 mg, 0.55 mmol), Xphos (262 mg, 0.55 mmol) and tert-butyl 2-(bromozincio)acetate (2.13 g, 8.66 mmol). The resulting solution was stirred for 30 min at RT. The resulting solution was allowed to react with stirring for an additional 3 h at 60° C. The resulting mixture was concentrated. The residue was eluted from silica gel with petroleum ether. This resulted in 360 mg (18.6%) of the title compound as a solid. MS-ESI: 355/357 (M+1).

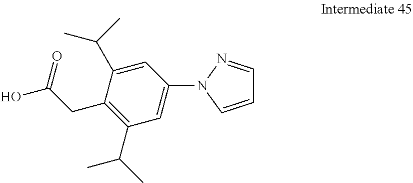 [SMILES: CC(C)c1cc(-n2cccn2)cc(C(C)C)c1CC(=O)O]

### 2-(2,6-diisopropyl-4-(1H-pyrazol-1-yl)phenyl)acetic Acid

### Step 3: Tert-butyl 2-(2,6-diisopropyl-4-(1H-pyrazol-1-yl)phenyl)acetate

Into a 50-mL round-bottom flask purged and maintained with an inert atmosphere of nitrogen, was placed a mixture of tert-butyl 2-(4-bromo-2,6-diisopropylphenyl)acetate (360 mg, 1.01 mmol) in dioxane (10 mL). To the stirred solution was added 1H-pyrazole (275 mg, 4.04 mmol), copper(I) iodide (76 mg, 0.40 mmol) and potassium phosphate (642 mg, 3.03 mmol). To the above (1R, 2R)-cyclohexane-1,2-diamine (0.05 mL, 0.40 mmol) was added dropwise. The resulting solution was refluxed overnight. The reaction was then concentrated and the residue was eluted from silica gel with ethyl acetate/petroleum ether (1:20). This resulted in 120 mg (35%) of the title compound as a yellow oil. MS-ESI: 342.2 (M+1).

### Step 4: 2-(2,6-Diisopropyl-4-(1H-pyrazol-1-yl)phenyl)acetic Acid

Into a 50-mL sealed tube purged and maintained with an inert atmosphere of nitrogen, was placed a solution of tert-butyl 2-(2,6-diisopropyl-4-(1H-pyrazol-1-yl)phenyl)acetate (120 mg, 0.35 mmol) in TFA (10 mL). The resulting solution was stirred overnight at RT. The reaction was then concentrated and used in the next step without purification. MS-ESI: 286.2 (M+1).

TABLE 15

| Intermediate | | | |
|---|---|---|---|
| # | Structure | IUPAC Name | Exact Mass [M + H]⁺ |
| Intermediate 46 | 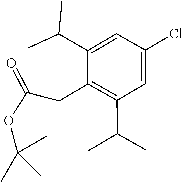 [SMILES: CC(C)c1cc(Cl)cc(C(C)C)c1CC(=O)OC(C)(C)C] | tert-butyl 2-(4-chloro-2,6-diisopropylphenyl)acetate | 311 |

The Intermediates in the following Table were prepared using the similar procedures for converting compound 176″ to Intermediate 44 shown in Scheme 33 from appropriated starting materials.

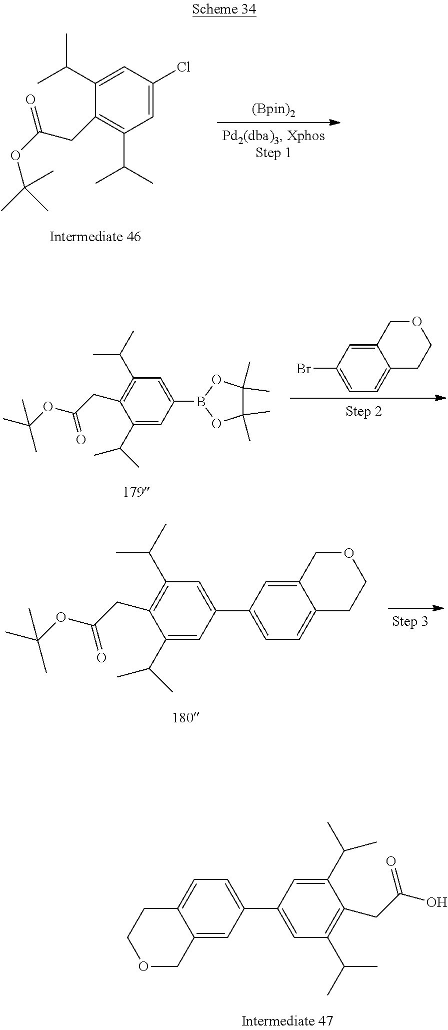

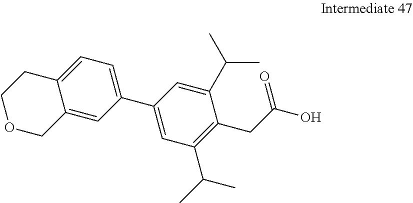 [SMILES: CC(C)c1cc(-c2ccc3c(c2)COCC3)cc(C(C)C)c1CC(=O)O]

### 2-(4-(Isochroman-7-yl)-2,6-diisopropylphenyl)acetic Acid

### Step 1: Tert-butyl 2-(2,6-diisopropyl-4-(4,4, 5, 5-tetramethyl-1,3,2-dioxaborolan-2-yl)phenyl)acetate

Into a 50-mL round-bottom flask purged and maintained with an inert atmosphere of nitrogen, was placed tert-butyl 2-[4-chloro-2,6-bis(propan-2-yl)phenyl]acetate (310 mg, 1.00 mmol) in dioxane (10 mL). To the stirred solution was added 4,4,4′,4′,5,5,5′,5′-octamethyl-2,2′-bi(1,3,2-dioxaborolane) (508 mg, 2.0 mmol), KOAc (195 mg, 1.99 mmol), Xphos (95.1 mg, 0.20 mmol) Pd₂(dba)₃ (91.3 mg, 0.10 mmol). The resulting solution was stirred for 16 h at 90° C. in an oil bath under nitrogen. Then the mixture was concentrated and the residue was eluted from silica gel with ethyl acetate/petroleum ether (1:20). This resulted in 400 mg (99.7%) of the title compound as a crude solid. MS-ESI: 403 (M+1).

### Step 2: Tert-butyl 2-(4-(isochroman-7-yl)-2,6-diisopropylphenyl)acetate

Into a 50-mL round-bottom flask purged and maintained with an inert atmosphere of nitrogen, was placed tert-butyl 2-(2,6-diisopropyl-4-(4,4,5,5-tetramethyl-1,3,2-dioxaborolan-2-yl)phenyl)acetate (402 mg, 1.00 mmol) in dioxane (10 mL) and H₂O (2.5 mL). To the stirred solution was added Cs₂CO₃ (652.0 mg, 2.00 mmol), 7-bromo-3,4-dihydro-1H-2-benzopyran (212.9 mg, 1.00 mmol) and Pd(dppf)Cl₂ (73.1 mg, 0.10 mmol). The resulting solution was stirred for 4 h at 80° C. in an oil bath. The resulting solution was diluted with 20 mL of ethyl acetate. The resulting mixture was washed with 2×20 mL of H₂O and 2×20 mL of brine. The residue was eluted from silica gel with ethyl acetate/petroleum ether (1:10). This resulted in 300 mg (73.4%) of the title compound as a light brown solid. MS-ESI: 409 (M+1).

### Step 3: 2-(4-(Isochroman-7-yl)-2,6-diisopropylphenyl)acetic Acid

Into a 25-mL round-bottom flask purged and maintained with an inert atmosphere of nitrogen, was placed tert-butyl 2-[4-(3,4-dihydro-1H-2-benzopyran-7-yl)-2,6-bis(propan-2-yl)phenyl]acetate (300 mg, 0.73 mmol) in DCM (4 mL) and TFA (1 mL). The resulting solution was stirred for 16 h at RT. The resulting mixture was concentrated. The residue was eluted from silica gel with ethyl acetate/petroleum ether (1:2). This resulted in 80 mg (30.9%) of the title compound as a light brown solid. MS-ESI: 351 (M−1).

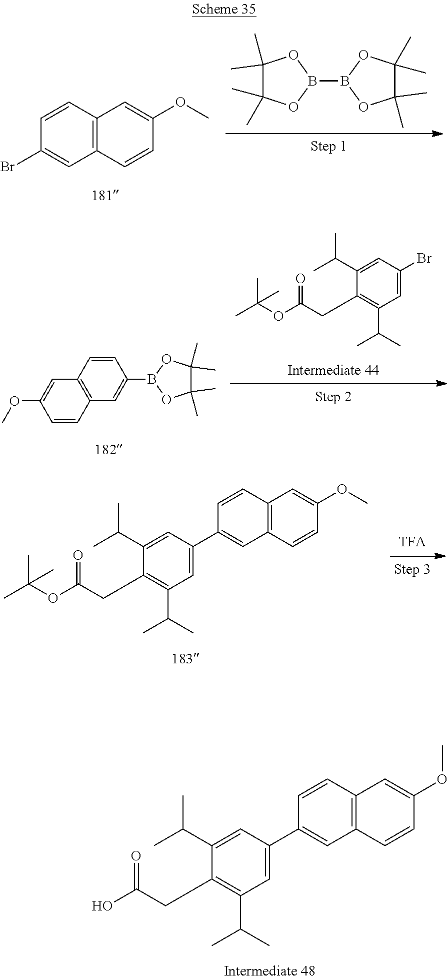

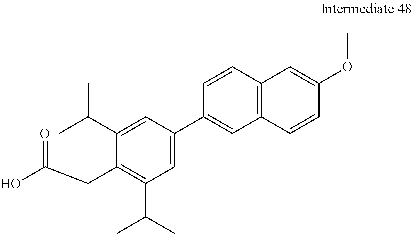 [SMILES: COc1ccc2cc(-c3cc(C(C)C)c(CC(=O)O)c(C(C)C)c3)ccc2c1]

### 2-(2,6-Diisopropyl-4-(6-methoxynaphthalen-2-yl)phenyl)acetic Acid

### Step 1: 2-(6-Methoxynaphthalen-2-yl)-4,4, 5, 5-tetramethyl-1,3,2-dioxaborolane

Into a 50-mL round-bottom flask, was placed 2-bromo-6-methoxynaphthalene (115 mg, 0.49 mmol) in dioxane (5 mL), to the stirred solution was added potassium acetate (175 mg, 1.27 mmol), 4,4, 5, 5-tetramethyl-2-(4,4, 5, 5-tetramethyl-1,3,2-dioxaborolan-2-yl)-1,3,2-dioxaborolane (113 mg, 0.45 mmol) and Pd(PPh₃)₂Cl₂ (9 mg). The resulting solution was stirred for 10 h at 110° C. The resulting solution was extracted with 3×20 mL of ethyl acetate and dried over anhydrous sodium sulfate. The solids were filtered out. The resulting mixture was concentrated under vacuum. This resulted in 120 mg of the title compound as an off-white solid. MS-ESI: 285 (M+1).

### Step 2: 2-(2,6-Diisopropyl-4-(6-methoxynaphthalen-2-yl)phenyl)acetic Acid

Into a 50-mL round-bottom flask, was placed 2-(6-methoxynaphthalen-2-yl)-4,4, 5, 5-tetramethyl-1,3,2-dioxaborolane (100 mg) in dioxane (15 mL) and H₂O (1.5 mL), to the stirred solution was added Cs₂CO₃ (344 mg), Pd(dppf)Cl₂ (27.5 mg), 2-[4-bromo-2,6-bis(propan-2-yl)phenyl] acetic acid (125 mg). The resulting solution was stirred for 15 h at 80° C. The resulting mixture was concentrated under vacuum. The residue was eluted from silica gel with ethyl acetate/petroleum ether (1:3). This resulted in 90 mg (58.9%) of the title compound as a yellow solid. MS-ESI: 433 (M+1).

### Step 3: 2-(2,6-Diisopropyl-4-(6-methoxynaphthalen-2-yl)phenyl)acetic Acid

Into a 50-mL round-bottom flask, was placed tert-butyl 2-[4-(6-methoxynaphthalen-2-yl)-2,6-bis(propan-2-yl)phenyl]acetate (80 mg, 0.18 mmol) in DCM (5 mL) and TFA (2.0 mL). The resulting solution was stirred for 1 h at RT. The resulting mixture was concentrated. This resulted in 80 mg (crude) of the title compound as a light yellow solid. MS-ESI: 377 [M+1]

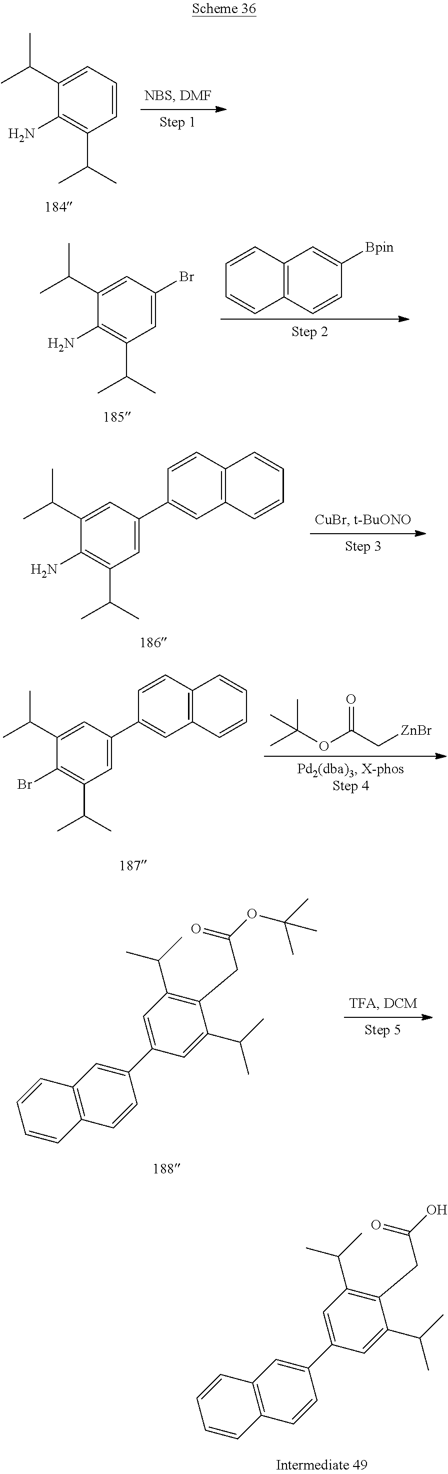

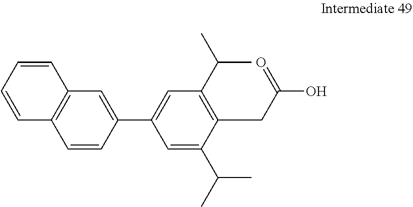 [SMILES: CC(C)c1cc(-c2ccc3ccccc3c2)cc(C(C)C)c1CC(=O)O]

### 2-(2,6-Diisopropyl-4-(naphthalen-2-yl)phenyl)acetic Acid

### Step 1: 4-Bromo-2,6-diisopropylaniline

Into a 250-mL 3-necked round-bottom flask purged and maintained with an inert atmosphere of nitrogen, was placed 2,6-bis(propan-2-yl)aniline (20 g, 56.4 mmol) in DMF (200 mL), to the stirred solution was added NBS (20.1 g, 112 mmol). The resulting solution was stirred for 6 h at RT. The resulting mixture was washed with 100 ml of water. The resulting solution was extracted with 3×100 ml of ethyl acetate dried over anhydrous sodium sulfate. The solids were filtered out. The resulting mixture was concentrated. The residue was eluted from silica gel with ethyl acetate/petroleum ether (1:1). This resulted in 16 g (55.3%) of the title compound as a white solid. MS-ESI: 256/258 (M+1).

### Step 2: 2,6-Biisopropyl-4-(naphthalen-2-yl)aniline

Into a 500-mL round-bottom flask purged and maintained with an inert atmosphere of nitrogen, was placed a solution of 4-bromo-2,6-bis(propan-2-yl)aniline (10 g, 39.0 mmol) in dioxane (250 mL) and H₂O (25 mL). To the stirred solution was added 4,4, 5, 5-tetramethyl-2-(naphthalen-2-yl)-1,3,2-dioxaborolane (11.9 g, 46.8 mmol), Pd(dppf)Cl₂ (7.81 g, 7.8 mmol) and Cs₂CO₃ (25.4 g, 78.1 mmol). The resulting solution was stirred for 10 min at RT. The resulting solution was then allowed to react for an additional 19 h at 80° C. The resulting mixture was concentrated. The residue was eluted from silica gel with ethyl acetate/petroleum ether (1:1). This resulted in 6.5 g (54.9%) of the title compound as a red solid. MS-ESI: 304 (M+1).

### Step 3: 2-(4-Bromo-3, 5-diisopropylphenyl)naphthalene

Into a 250-mL round-bottom flask purged and maintained with an inert atmosphere of nitrogen, was placed a solution of 4-(naphthalen-2-yl)-2,6-bis(propan-2-yl)aniline (6.0 g, 19.8 mmol) in ACN (100 mL). To the stirred solution was added tert-butyl nitrite (4.08 g, 39.5 mmol) and CuBr (5.67 g, 39.5 mmol). The resulting solution was stirred for 30 min at RT. The resulting solution was allowed to react with stirring for an additional 180 min at 60° C. The mixture was concentrated and the residue was eluted from silica gel with PE. This resulted in 105 mg (17.3%) of the title compound as a red solid. MS-ESI: 367/369 (M+1).

### Step 4: Tert-butyl 2-(2,6-diisopropyl-4-(naphthalen-2-yl)phenyl)acetate

Into a 100-mL round-bottom flask purged and maintained with an inert atmosphere of nitrogen, was placed a solution of 2-[4-bromo-3, 5-bis(propan-2-yl)phenyl]naphthalene (2 g, 5.44 mmol) in THF (50 mL), to the above solution was added XPhos (0.3 g, 0.54 mmol), and Pd₂(dba)₃CH₂Cl₂ (0.2 g, 0.27 mmol). The resulted solution was stirred for 15 min at RT. Then to the mixture was added tert-butyl 2-(bromozincio)acetate (2.8 g, 10.9 mmol) with stirring. The resulting solution was allowed to react for an additional 180 min at 65° C. The residue was eluted from silica gel with PE. This resulted in 1.0 g (45.6%) of the title compound as a yellow solid. MS-ESI: 403 (M+1).

### Step 5: 2-(2,6-Diisopropyl-4-(naphthalen-2-yl)phenyl)acetic Acid

Into a 250-mL round-bottom flask, was placed tert-butyl 2-[4-(naphthalen-2-yl)-2,6-bis(propan-2-yl)phenyl]acetate (2.48 g, 6.16 mmol) in TFA (20 mL) and DCM (20 mL). The resulting solution was stirred for 5 h at RT. Then the mixture was concentrated. The residue was eluted from silica gel with ethyl acetate/petroleum ether (13/100). This resulted in 1.68 g (78.5%) of the title compound as a yellow solid. MS-ESI: 347 (M+1).

TABLE 16

| | | | Exact |
|---|---|---|---|
| Intermediate | | | Mass |
| # | Structure | IUPAC Name | [M − H]⁻ |
| Intermediate 50 | 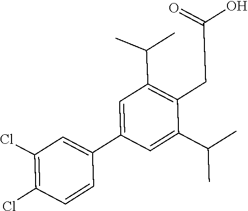 [SMILES: CC(C)c1cc(-c2ccc(Cl)c(Cl)c2)cc(C(C)C)c1CC(=O)O] | 2-(3′,4′-dichloro-3,5-diisopropyl-[1,1′-biphenyl]-4-yl)acetic acid | 363 |
| Intermediate 51 | 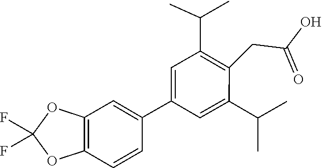 [SMILES: CC(C)c1cc(-c2ccc3c(c2)OC(F)(F)O3)cc(C(C)C)c1CC(=O)O] | 2-(4-(2,2-difluorobenzo[d][1,3]dioxol-5-yl)-2,6- diisopropylphenyl)acetic acid | 375 |
| Intermediate 52 | 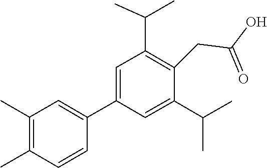 [SMILES: Cc1ccc(-c2cc(C(C)C)c(CC(=O)O)c(C(C)C)c2)cc1C] | 2-(3, 5-diisopropyl-3′,4′-dimethyl-[1,1′-biphenyl]-4-yl)acetic acid | 323 |
| Intermediate 53 | 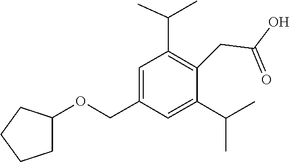 [SMILES: CC(C)c1cc(COC2CCCC2)cc(C(C)C)c1CC(=O)O] | 2-(4-((cyclopentyloxy)methyl)-2,6-diisopropylphenyl)acetic acid | 317 |

The Intermediates in the following Table were prepared using the similar procedures for converting compound 184″ to Intermediate 49 shown in Scheme 36 from appropriated starting materials.

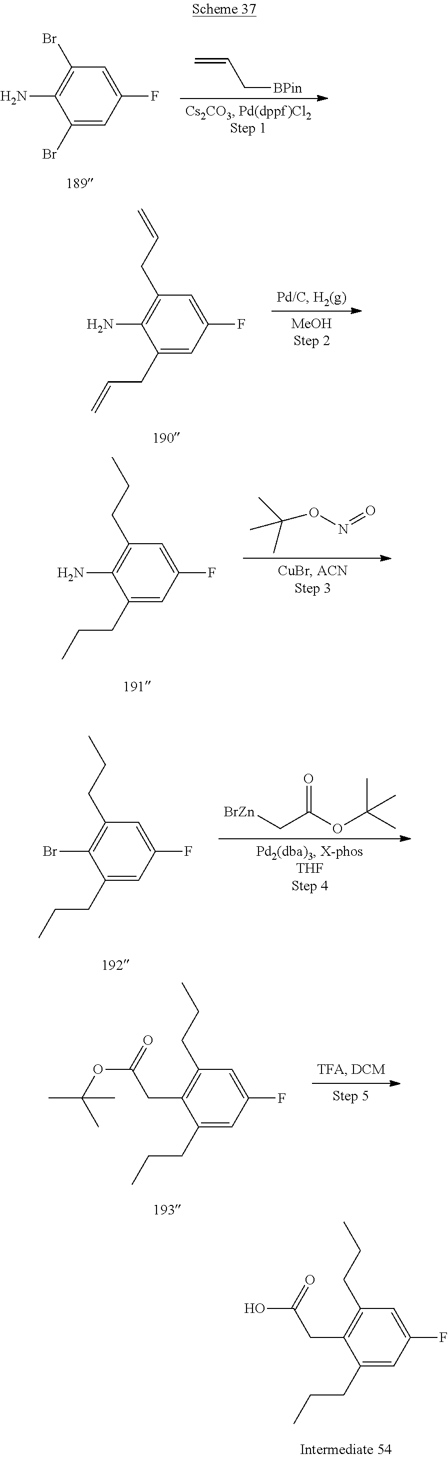

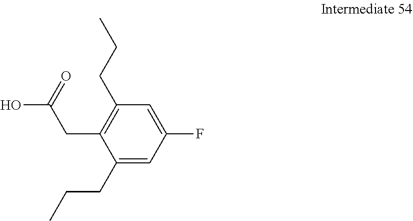 [SMILES: CCCc1cc(F)cc(CCC)c1CC(=O)O]

### 2-(4-Fluoro-2,6-dipropylphenyl)acetic Acid

### Step 1: 2,6-Diallyl-4-fluoroaniline

Into a 250-mL round-bottom flask purged and maintained with an inert atmosphere of nitrogen, was placed 2-bromo-4-fluoro-6-(prop-2-en-1-yl)aniline (3.7 g, 16.1 mmol) in THF (100 mL), to the stirred solution was added 4,4, 5, 5-tetramethyl-2-(prop-2-en-1-yl)-1,3,2-dioxaborolane (8.1 g, 48.2 mmol), Cs₂CO₃ (15.7 g, 48.2 mmol) and Pd(dppf)Cl₂ (588 mg, 0.80 mmol). The resulting solution was stirred overnight at 70° C. in an oil bath. The resulting mixture was concentrated under vacuum. The residue was eluted from silica gel with ethyl acetate/petroleum ether (1:10). This resulted in 2.6 g (84.5%) of the title compound as yellow oil. MS-ESI: 192 [M+1].

### Step 2: 4-Fluoro-2,6-dipropylaniline

Into a 100-mL round-bottom flask, was placed 4-fluoro-2,6-bis(prop-2-en-1-yl)aniline (2.6 g, 13.59 mmol) in methanol (50 mL). To the stirred solution was added Pd/C (10% wt., 300 mg). The flask was evacuated and filled three times with hydrogen. The resulting solution was stirred 5 h at RT under hydrogen. The solids were filtered out. The resulting mixture was concentrated under vacuum. This resulted in 2.5 g (94.1%) of the title compound as light yellow oil. MS-ESI: 196 [M+1]

### Step 3: 2-Bromo-5-fluoro-1,3-dipropylbenzene

Into a 100-mL round-bottom flask purged and maintained with an inert atmosphere of nitrogen, was placed 4-fluoro-2,6-dipropylaniline (840 mg, 4.30 mmol) in ACN (20 mL). To the stirred solution was added CuBr (1.2 g, 8.60 mmol) and tert-butyl nitrite (888 mg, 8.61 mmol). The resulting solution was stirred for 3 h at 60° C. in an oil bath. The resulting mixture was concentrated under vacuum. The residue was eluted from silica gel with petroleum ether. This resulted in 640 mg (57.4%) of the title compound as light yellow oil. MS-ESI: 259/261[M+1].

### Step 4: Tert-butyl 2-(4-fluoro-2,6-dipropylphenyl)acetate

Into a 100-mL round-bottom flask purged and maintained with an inert atmosphere of nitrogen, was placed 2-bromo-5-fluoro-1,3-dipropylbenzene (460 mg, 1.77 mmol) in THF (10 mL). To the mixture was added Xphos (85 mg, 0.18 mmol) and Pd₂(dba)₃ (82 mg, 0.09 mmol). The resulting solution was stirred for 30 min at RT. Then to the above was added tert-butyl 2-(bromozincio)acetate (1.4 g, 5.32 mmol). The resulting solution was stirred for 3 h at 65° C. in an oil bath. The reaction was then quenched by the addition of 10 mL of NH₄Cl (sat.). The resulting solution was extracted with 3×10 mL of DCM. The organic layers were combined, dried over anhydrous sodium sulfate, and concentrated under vacuum. This resulted in 300 mg (57.4%) of the title compound as light yellow oil. MS-ESI: 295 [M+1].

### Step 5: 2-(4-Fluoro-2,6-dipropylphenyl)acetic Acid

Into a 50-mL round-bottom flask, was placed tert-butyl 2-(4-fluoro-2,6-dipropylphenyl)acetate (300 mg) in DCM (4 mL) and TFA (2 mL). The resulting solution was stirred for 2 h at RT. The resulting mixture was concentrated under vacuum. The crude product was purified by Prep-TLC with ethyl acetate/petroleum ether (1:3). This resulted in 165 mg (67.9%) of the title compound as a light yellow solid. MS-ESI: 239 [M+1]

TABLE 17

| | | | Exact |
|---|---|---|---|
| Intermediate | | | Mass |
| # | Structure | IUPAC Name | [M + H]⁺ |
| Intermediate 55 | 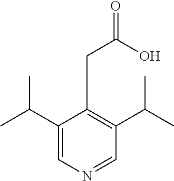 [SMILES: CC(C)c1cncc(C(C)C)c1CC(=O)O] | 2-(3,5-diisopropylpyridin-4-yl)acetic acid | 222 |
| Intermediate 56 | 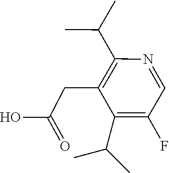 [SMILES: CC(C)c1ncc(F)c(C(C)C)c1CC(=O)O] | 2-(5-fluoro-2,4-diisopropylpyridin-3-yl)acetic acid | 240 |
| Intermediate 57 | 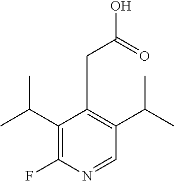 [SMILES: CC(C)c1cnc(F)c(C(C)C)c1CC(=O)O] | 2-(2-fluoro-3,5-diisopropylpyridin-4-yl)acetic acid | 240 |

The Intermediates in the following Table were prepared using the similar procedures for converting compound 189″ to Intermediate 54 shown in Scheme 37 from appropriated starting materials.

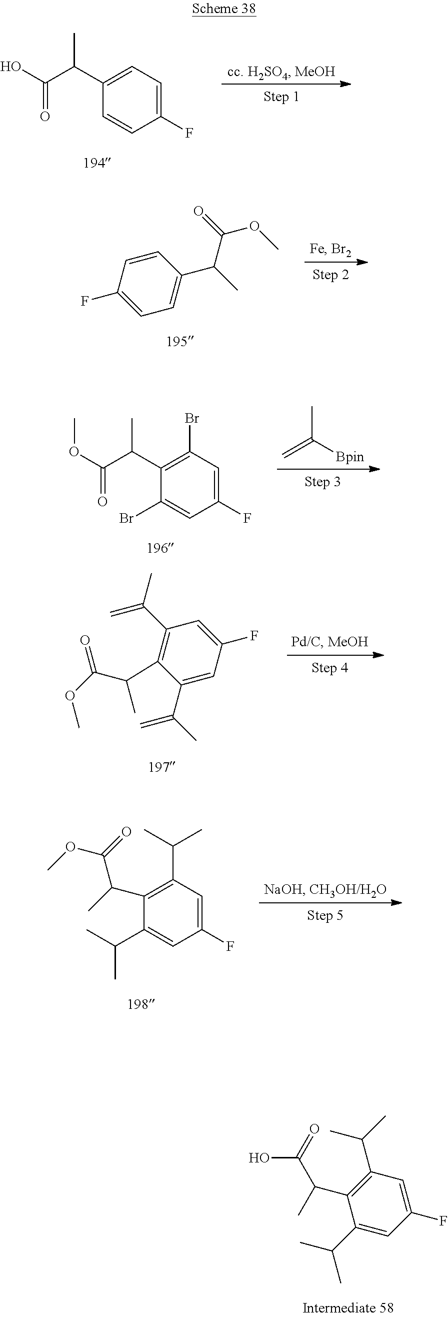

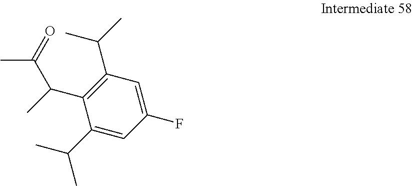 [SMILES: CC(=O)C(C)c1c(C(C)C)cc(F)cc1C(C)C]

### 2-(4-Fluoro-2,6-diisopropylphenyl)propanoic Acid

### Step 1: Methyl 2-(4-fluorophenyl)propanoate

Into a 100-mL round-bottom flask, was placed a solution of 2-(4-fluorophenyl)propanoic acid (2 g, 11.89 mmol) in methanol (20 mL). To the mixture conc. H₂SO₄ (0.05 mL) was added. The resulting solution was stirred for 16 h at 85° C. The reaction was then quenched by the addition of 100 mL of water. The resulting solution was extracted with 3×50 mL of dichloromethane and the organic layers combined. The solids were filtered out. The mixture was dried over anhydrous sodium sulfate and concentrated under vacuum. This resulted in 2.1 g (97%) of the title compound as yellow oil. MS-ESI: 183 [M+1].

### Step 2: Methyl 2-(2,6-dibromo-4-fluorophenyl)propanoate

Into a 100-mL round-bottom flask purged and maintained with an inert atmosphere of nitrogen, was placed a solution of methyl 2-(4-fluorophenyl)propanoate (1.7 g, 9.33 mmol) in CHCl₃ (20 mL). To the stirred solution was added Fe powder (0.21 g) and Br₂ (1.92 mL). The resulting solution was stirred for 16 h at 50° C. The reaction was then quenched by the addition of 50 mL of saturated Na₂S₂O₃ solution. The resulting solution was extracted with 3×50 mL of DCM and the organic layers were combined, dried over anhydrous sodium sulfate and concentrated under vacuum. This resulted in 1.03 g (32%) of the title compound as yellow crude oil. MS-ESI: 339/341 [M+1].

### Step 3: Methyl 2-(4-fluoro-2,6-di(prop-1-en-2-yl)phenyl)propanoate

Into a 40-mL sealed tube purged and maintained with an inert atmosphere of nitrogen, was placed a solution of methyl 2-(2,6-dibromo-4-fluorophenyl)propanoate (1.03 g, 3.03 mmol) in dioxane (10 mL) and H₂O (1 mL). To the stirred solution was added Cs₂CO₃ (2 g, 6.14 mmol), Pd(dppf)Cl₂ (230 mg, 0.31 mmol) and 4,4,5,5-tetramethyl-2-(prop-1-en-2-yl)-1,3,2-dioxaborolane (1.07 g, 6.37 mmol). The resulting solution was stirred for 6 h at 110° C. The resulting mixture was concentrated under vacuum. The residue was eluted from silica gel with ethyl acetate/petroleum ether (1:6). This resulted in 754 mg (95%) of the title compound as yellow oil. MS-ESI: 263 [M+1].

### Step 4: Methyl 2-(4-fluoro-2,6-diisopropylphenyl)propanoate

Into a 50-mL round-bottom flask purged and maintained with an inert atmosphere of H₂, was placed a solution of methyl 2-[4-fluoro-2,6-bis(prop-1-en-2-yl)phenyl]propanoate (820 mg, 3.13 mmol) in methanol (20 mL). To the stirred solution was added Pd/C (10% wt., 0.2 g). The resulting solution was stirred for 4 h at RT. The solids were filtered out. The resulting mixture was concentrated under vacuum. This resulted in 700 mg (84%) of the title compound as yellow crude oil. MS-ESI: 267 [M+1.

### Step 5: 2-(4-Fluoro-2,6-diisopropylphenyl)propanoic Acid

Into a 40-mL sealed tube, was placed a solution of methyl 2-[4-fluoro-2,6-bis(propan-2-yl)phenyl]propanoate (300 mg, 1.13 mmol) in 6 M sodium hydroxide (3 mL) and MeOH (3 mL). The resulting solution was stirred for 3 h at 90° C. The reaction was then quenched by the addition of 50 mL of water. The pH value of the solution was adjusted to 2 with hydrogen chloride (1 M). The resulting solution was extracted with 2×50 mL of ethyl acetate and the organic layers combined. The solids were filtered out. The mixture was dried over anhydrous sodium sulfate and concentrated under vacuum. This resulted in 150 mg (53%) of the title compound as yellow oil. MS-ESI: 253 [M+1].

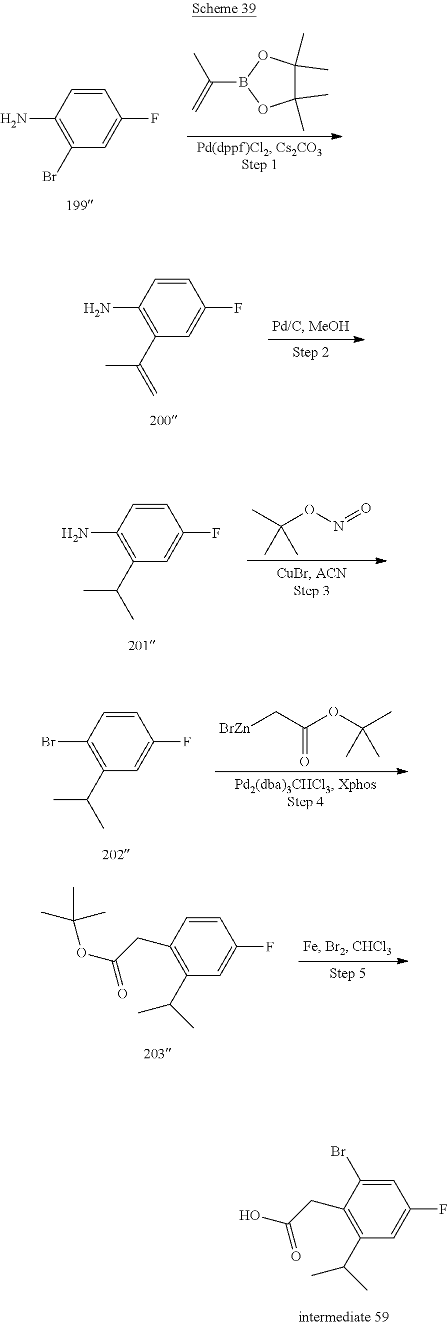

Steps 1-4 used similar procedures for converting compound 189″ to compound 193″ shown in Scheme 37 to afford compound 203″ from compound 199″. MS-ESI: 253 (M+1).

### Step 5: 2-(2-bromo-4-fluoro-6-isopropylphenyl)acetic Acid

Into a 100-mL 3-necked round-bottom flask purged and maintained with an inert atmosphere of nitrogen, was placed tert-butyl 2-[4-fluoro-2-(propan-2-yl)phenyl]acetate (1.0 g, 3.96 mmol) in CHCl₃ (25 mL). To the solution was added AcOH (0.01 mL), Fe powder (22.1 mg, 0.40 mmol) and Br₂ (3.17 g, 19.8 mmol). The resulting solution was stirred for 16 h at 50° C. in an oil bath. The reaction was then quenched by the addition of 20 mL of Na₂S₂O₃. The resulting solution was extracted with 2×20 ml of ethyl acetate concentrated. The residue was eluted from silica gel with ethyl acetate/petroleum ether (1:2). This resulted in 700 mg (64.2%) of the title compound as a white solid. MS-ESI: 275 [M+1].

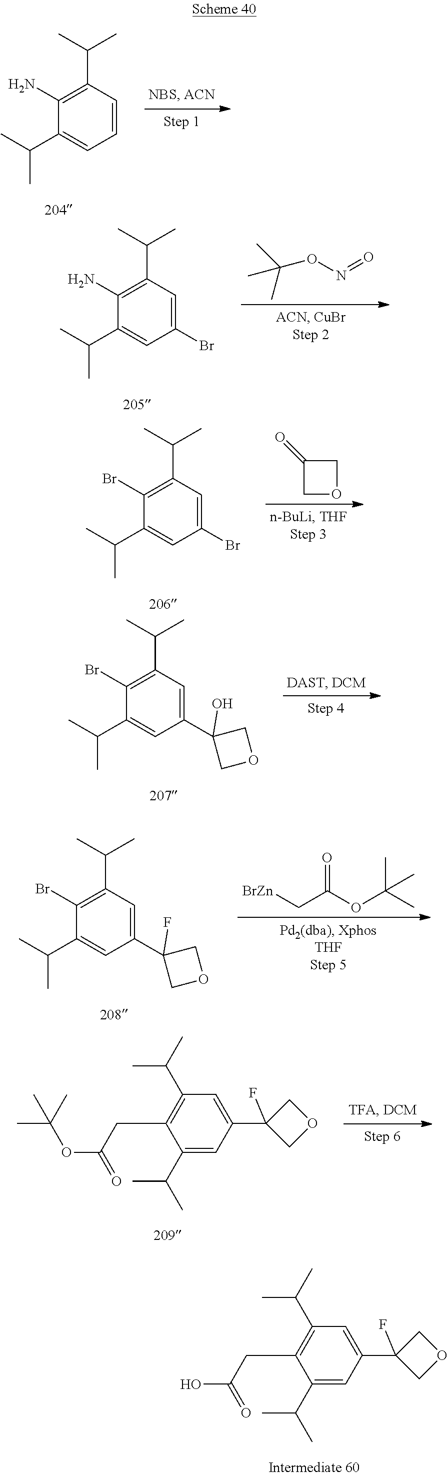

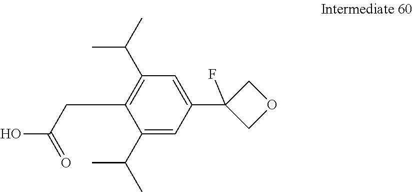 [SMILES: CC(C)c1cc(C2(F)COC2)cc(C(C)C)c1CC(=O)O]

### 2-(4-(3-Fluorooxetan-3-yl)-2,6-diisopropylphenyl)acetic Acid

### Step 1: 4-Bromo-2,6-diisopropylaniline

Into a 500-mL round-bottom flask, was placed 2,6-bis(propan-2-yl)aniline (10 g, 56.4 mmol) in ACN (200 mL), to the stirred solution was added NBS (11.0 g, 62.0 mmol). The resulting solution was stirred overnight at RT. The resulting mixture was concentrated under vacuum. The residue was eluted from silica gel with petroleum ether. This resulted in 9.5 g (65.7%) of the title compound as brown oil. MS-ESI: 256/258 [M+1].

### Step 2: 2, 5-Dibromo-1,3-diisopropylbenzene

Into a 500-mL round-bottom flask purged and maintained with an inert atmosphere of nitrogen, was placed 4-bromo-2,6-bis(propan-2-yl)aniline (6.4 g, 24.9 mmol) in ACN (200 mL). To the stirred solution was added CuBr (7.2 g, 50.2 mmol) and tert-butyl nitrite (5.2 g, 50.5 mmol). The resulting solution was stirred for 3 h at 65° C. in an oil bath. The resulting mixture was concentrated under vacuum. The residue was eluted from silica gel with petroleum ether. This resulted in 5 g (62.5%) of the title compound as light yellow oil. MS-ESI: 319/321/323 [M+1].

### Step 3: 3-(4-Bromo-3, 5-diisopropylphenyl)oxetan-3-ol

Into a 100-mL 3-necked round-bottom flask purged and maintained with an inert atmosphere of nitrogen, was placed 2, 5-dibromo-1,3-bis(propan-2-yl)benzene (5 g, 15.6 mmol) in THF (50 mL). This was followed by the addition of n-BuLi (2.5 M, 6.25 mL, 15.6 mmol) dropwise with stirring at −78° C. The resulting solution was stirred for 30 min at −78° C. To the above was added a solution of oxetan-3-one (1.13 g, 15.6 mmol) in THF (2 mL) dropwise with stirring at −78° C. The resulting solution was slowly warmed to RT and stirred for 2 h at RT. The reaction was then quenched by the addition of 100 mL of NH₄Cl (sat.). The resulting solution was extracted with 3×100 mL of ethyl acetate and the organic layers combined and dried over anhydrous sodium sulfate and concentrated under vacuum. The residue was eluted from silica gel with ethyl acetate/petroleum ether (1:3). The crude product was purified by Flash-Prep-HPLC with the following conditions: Column, Cis silica gel; mobile phase, H₂O (0.1% FA) and ACN (40% to 70% ACN gradient in 30 min), Detector, UV 254/210 nm. This resulted in 1.25 g (25.5%) of the title compound as a white solid. MS-ESI: 313/315[M+1].

### Step 4: 3-(4-Bromo-3, 5-diisopropylphenyl)-3-fluorooxetane

Into a 50-mL 3-necked round-bottom flask purged and maintained with an inert atmosphere of nitrogen, was placed 3-[4-bromo-3, 5-bis(propan-2-yl)phenyl]oxetan-3-ol (600 mg, 1.92 mmol) in DCM (10 mL). This was followed by the addition of DAST (618 mg, 3.83 mmol) dropwise with stirring at 0° C. The resulting solution was stirred overnight at RT. The reaction was then quenched by the addition of 5 mL of methanol. The resulting mixture was concentrated under vacuum. The residue was eluted from silica gel with ethyl acetate/petroleum ether (1:10). This resulted in 430 mg (71.2%) of the title compound as a white solid. MS-ESI: 315/317[M+1].

### Step 5: Tert-butyl 2-(4-(3-fluorooxetan-3-yl)-2,6-diisopropylphenyl)acetate

Into a 100-mL round-bottom flask, was placed 3-[4-bromo-3, 5-bis(propan-2-yl)phenyl]-3-fluorooxetane (420 mg, 1.33 mmol) in THF (20 mL), to the mixture was added Xphos (60 mg, 0.13 mmol) and Pd₂(dba)₃ (61 mg, 0.07 mmol). The resulting solution was stirred for 30 min at RT. Then to the above was added tert-butyl 2-(bromozincio)acetate (694.0 mg, 2.66 mmol). The resulting solution was stirred for 3 h at 60° C. in an oil bath. The resulting mixture was concentrated under vacuum. The residue was eluted from silica gel with ethyl acetate/petroleum ether (1:10). resulted in 450 mg (96.3%) of the title compound as a light yellow solid. MS-ESI: 351 [M+1].

### Step 6: 2-(4-(3-Fluorooxetan-3-yl)-2,6-diisopropylphenyl)acetic Acid

Into a 50-mL round-bottom flask, was placed tert-butyl 2-[4-(3-fluorooxetan-3-yl)-2,6-bis(propan-2-yl) phenyl]acetate (450 mg, 1.28 mmol) in DCM (4 mL) and TFA (2 mL). The resulting solution was stirred overnight at RT. The resulting mixture was concentrated under vacuum. The residue was eluted from silica gel with ethyl acetate/petroleum ether (1:3). This resulted in 300 mg (79.3%) of the title compound as a light yellow solid. MS-ESI: 295[M+1].

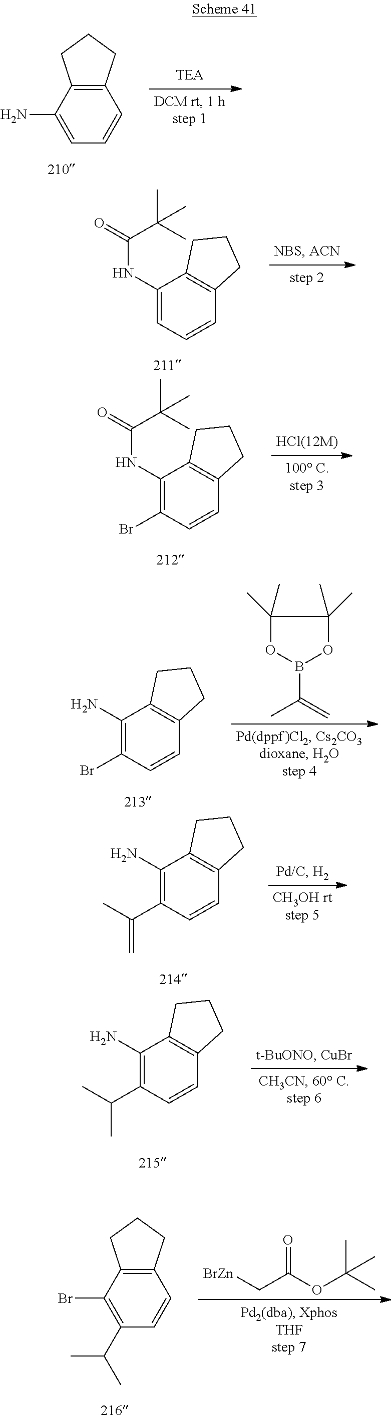

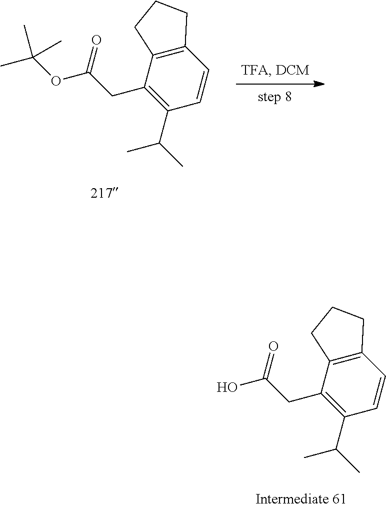 [SMILES: CC(C)c1ccc2c(c1CC(=O)OC(C)(C)C)CCC2 ; CC(C)c1ccc2c(c1CC(=O)O)CCC2]

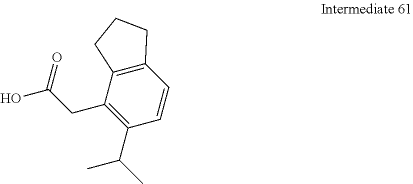 [SMILES: CC(C)c1ccc2c(c1CC(=O)O)CCC2]

### 2-(5-Isopropyl-2,3-dihydro-1H-inden-4-yl)acetic Acid

### Step 1: N-(2,3-dihydro-1H-inden-4-yl)pivalamide

Into a 500-mL round-bottom flask, was placed 2,3-dihydro-1H-inden-4-amine (10 g, 75.1 mmol) in DCM (100 mL), to the stirred solution was added 2,2-dimethylpropanoyl chloride (9.05 g, 75.1 mmol) and TEA (11.4 g, 112 mmol). The resulting solution was stirred for 1 h at RT. The reaction was then quenched by the addition of 100 mL of water. The resulting solution was extracted with 3×100 ml of DCM and dried over anhydrous sodium sulfate and concentrated. This resulted in 15 g (91.9%) of the title compound as an off-white solid. MS-ESI: 218 [M+1].

### Step 2: N-(5-bromo-2,3-dihydro-1H-inden-4-yl)pivalamide

Into a 500-mL round-bottom flask purged and maintained with an inert atmosphere of nitrogen, was placed a solution of N-(2,3-dihydro-1H-inden-4-yl)-2,2-dimethylpropanamide (9 g, 41.5 mmol) in ACN (200 mL). To above solution was added NBS (8.86 g, 49.8 mmol). The resulting solution was stirred for 15 h at RT, after which it was extracted with 3×200 ml of DCM. The organic layers were combined, washed with 3×200 ml of aq. Na₂CO₃, dried over anhydrous sodium sulfate, and concentrated. This resulted in 12 g of the title compound as a brown solid. MS-ESI: 296/298 [M+1]

### Step 3: 5-Bromo-2,3-dihydro-1H-inden-4-amine

Into a 500-mL round-bottom flask, was placed a solution of N-(5-bromo-2,3-dihydro-1H-inden-4-yl)-2,2-dimethylpropanamide (10 g, 33.8 mmol) in HCl (200 mL). The resulting solution was stirred for 15 h at 100° C. The reaction was then quenched by the addition of water. The resulting solution was extracted with 3×500 mL of ethyl acetate dried over anhydrous sodium sulfate and concentrated under vacuum. The residue was eluted from silica gel with ethyl acetate/petroleum ether (1:10). This resulted in 7 g (97.7%) of the title compound as a brown solid. MS-ESI: 212/214[M+1].

### Step 4: 5-(Prop-1-en-2-yl)-2,3-dihydro-1H-inden-4-amine

Into a 500-mL round-bottom flask purged and maintained with an inert atmosphere of nitrogen, was placed a solution of 5-bromo-2,3-dihydro-1H-inden-4-amine (7 g, 33 mmol) in dioxane (250 mL) and H₂O (25 mL). To the above was added 4,4, 5, 5-tetramethyl-2-(prop-1-en-2-yl)-1,3,2-dioxaborolane (8.32 g, 49.5 mmol), Cs₂CO₃ (32.2 g, 99.0 mmol) and Pd(dppf)Cl₂ (2.41 g, 3.3 mmol). The resulting solution was stirred for 15 h at 95° C. The reaction was then quenched by the addition of water. The resulting solution was extracted with 3×200 mL of DCM and concentrated. The residue was eluted from silica gel with ethyl acetate/petroleum ether (1:10). This resulted in 4 g (69.9%) of the title compound as a brown solid. MS-ESI: 174 [M+1].

### Step 5: 5-Isopropyl-2,3-dihydro-1H-inden-4-amine

Into a 250-mL round-bottom flask, was placed a solution of 5-(prop-1-en-2-yl)-2,3-dihydro-1H-inden-4-amine (4 g, 23.09 mmol) in methanol (100 mL), to the stirred solution was added Pd/C (10% wt., 400 mg). The flask was evacuated and flushed three times with hydrogen. The resulting solution was stirred overnight at RT under hydrogen. The solids were filtered out. The resulting mixture was concentrated under vacuum. This resulted in 4 g (98.8%) of the title compound as a brown solid. MS-ESI: 176 [M+1].

Steps 6-8 used similar procedures for converting compound 191″ to intermediate 54 shown in Scheme 37 to afford intermediate 61 from compound 215″. MS-ESI: 219 (M+1).

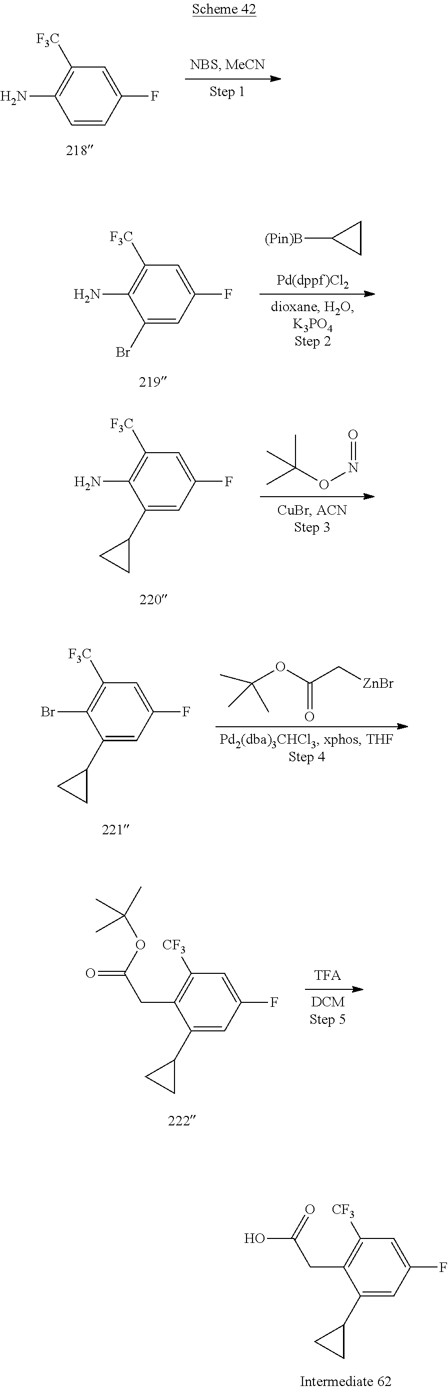

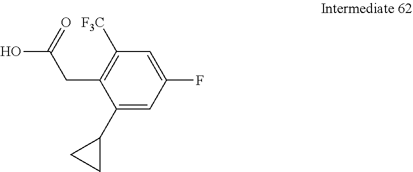 [SMILES: O=C(O)Cc1c(C2CC2)cc(F)cc1C(F)(F)F]

### 2-(2-Cyclopropyl-4-fluoro-6-(trifluoromethyl)phenyl)acetic Acid

### Step 1: 2-Bromo-4-fluoro-6-(trifluoromethyl)aniline

Into a 250-mL round-bottom flask, was placed 4-fluoro-2-(trifluoromethyl)aniline (11.6 g, 64.7 mmol) in ACN (100 mL). This was followed by the addition of NBS (12.6 mg, 71.2 mmol) in portions with stirring at 0° C. The resulting solution was stirred for 2 h at RT. The reaction was then quenched by the addition of water. The resulting solution was extracted with 3×100 mL of ethyl acetate. The residue was eluted from silica gel with ethyl acetate/petroleum ether (1:20). This resulted in 13 g (77.8%) of the title compound as a red solid. MS-ESI: 258/260 (M+1).

### Step 2: 2-Cyclopropyl-4-fluoro-6-(trifluoromethyl)aniline

Into a 500-mL round-bottom flask purged and maintained with an inert atmosphere of nitrogen, was placed 2-bromo-4-fluoro-6-(trifluoromethyl)aniline (10 g, 38.7 mmol) in dioxane (200 mL) and H₂O (10 mL). To the stirred solution was added K₃PO₄ (24.6 g, 116.2 mmol), Pd(dppf)Cl₂ (2.84 g, 3.88 mmol) and cyclopropylboronic acid or ester (4.99 g, 58.1 mmol). The resulting solution was stirred for overnight at 90° C. in an oil bath. The solids were filtered out. The resulting mixture was concentrated. The residue was eluted from silica gel with ethyl acetate/petroleum ether (1:20). This resulted in 7.5 g (88.2%) of the title compound as a yellow oil. MS-ESI: 220 (M+1).

### Step 3: 2-Bromo-1-cyclopropyl-5-fluoro-3-(trifluoromethyl)benzene

Into a 100-mL round-bottom flask, was placed 2-cyclopropyl-4-fluoro-6-(trifluoromethyl)aniline (1.5 g, 6.85 mmol) in ACN (30 mL). To the above solution was added tert-butyl nitrite (1.41 g, 13.7 mmol) and CuBr (1.96 g, 13.7 mmol). The resulting solution was stirred for 3 h at 60° C. The resulting mixture was concentrated. The residue was eluted from silica gel with ethyl acetate/petroleum ether (1:20). This resulted in 1 g (51.6%) of the title compound as a yellow liquid. MS-ESI: 283/285 (M+1).

### Step 4: Tert-butyl 2-[2-cyclopropyl-4-fluoro-6-(trifluoromethyl)phenyl]acetate

Into a 100-mL round-bottom flask purged and maintained with an inert atmosphere of nitrogen, was placed 2-bromo-1-cyclopropyl-5-fluoro-3-(trifluoromethyl)benzene (360 mg, 1.27 mmol) in THF (10 mL), Xphos (121.26 mg, 0.25 mmol), Pd₂(dba)₃.CHCl₃ (131.6 mg, 0.13 mmol), and tert-butyl 2-(bromozincio)acetate (662.4 mg, 2.54 mmol). The resulting solution was stirred for 2 h at 65° C. The resulting mixture was concentrated. The residue was eluted from silica gel with ethyl acetate/petroleum ether (1:20). This resulted in 300 mg (74.1%) of the title compound as yellow oil. MS-ESI: 319 (M+1).

### Step 5: 2-[2-Cyclopropyl-4-fluoro-6-(trifluoromethyl)phenyl]acetic Acid

Into a 50-mL round-bottom flask, was placed tert-butyl 2-[2-cyclopropyl-4-fluoro-6-(trifluoromethyl) phenyl]acetate (300 mg, 0.94 mmol) in TFA (2 mL) and DCM (2 mL). The resulting solution was stirred for 2 h at RT. The resulting mixture was concentrated. The residue was eluted from silica gel with ethyl acetate/petroleum ether (1:5). This resulted in 230 mg (93.0%) of the title compound as a yellow solid. MS-ESI: 263 (M+1).

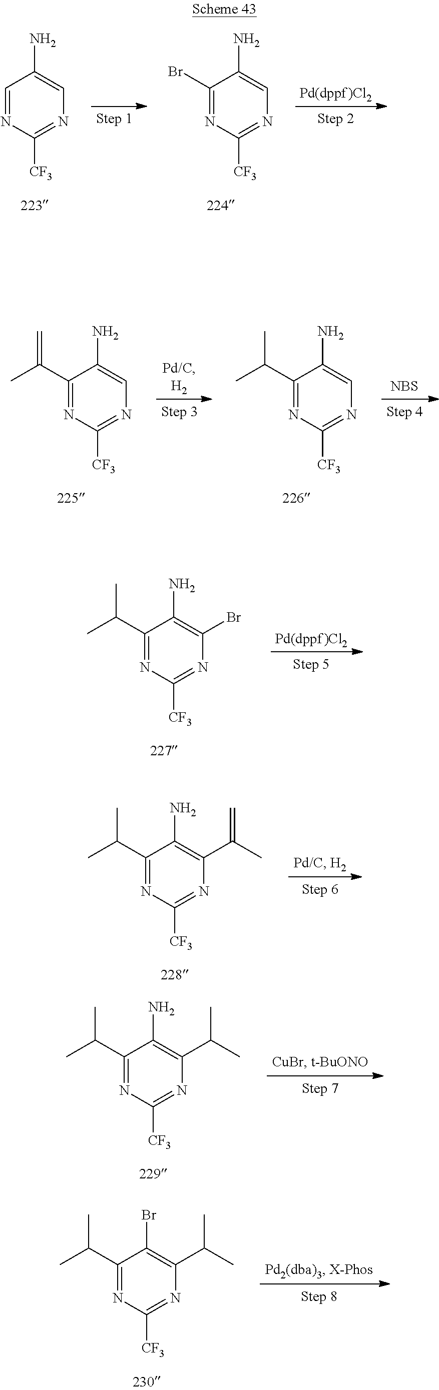

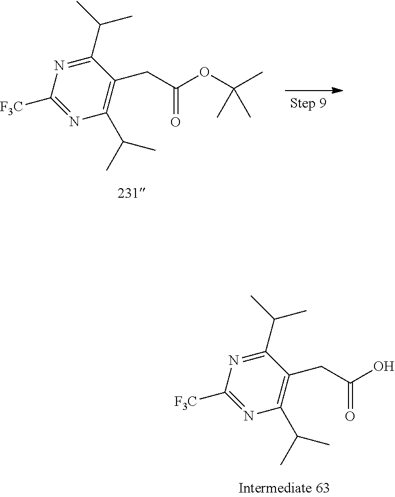 [SMILES: CC(C)c1nc(C(F)(F)F)nc(C(C)C)c1CC(=O)OC(C)(C)C ; CC(C)c1nc(C(F)(F)F)nc(C(C)C)c1CC(=O)O]

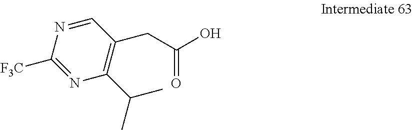 [SMILES: CC(C)c1nc(C(F)(F)F)ncc1CC(=O)O]

### 2-(4,6-Diisopropyl-2-(trifluoromethyl)pyrimidin-5-yl)acetic Acid

### Step 1: 4-Bromo-2-(trifluoromethyl)pyrimidin-5-amine

Into a 100-mL round-bottom flask purged and maintained with an inert atmosphere of nitrogen, was placed 2-(trifluoromethyl)pyrimidin-5-amine (2 g, 12.3 mmol) in acetonitrile (20 mL), to this stirred solution was added NBS (2.62 g, 14.7 mmol). The resulting solution was stirred for 12 h at RT. The resulting solution was diluted with 40 mL of water. The resulting solution was extracted with 2×30 mL of dichloromethane and concentrated. The residue was eluted from silica gel with ethyl acetate/petroleum ether (1:50 to 1:20). This resulted in 1.6 g (53.9%) of the title compound as a brown solid. MS-ESI: 242/244 [M+1]

### Step 2: 4-(Prop-1-en-2-yl)-2-(trifluoromethyl)pyrimidin-5-amine

Into a 100-mL round-bottom flask purged and maintained with an inert atmosphere of nitrogen, was placed 4-bromo-2-(trifluoromethyl)pyrimidin-5-amine (1.6 g, 6.61 mmol) in dioxane (20 mL). This was followed by the addition of 4,4,5,5-tetramethyl-2-(prop-1-en-2-yl)-1,3,2-dioxaborolane (1.44 g, 8.57 mmol), Pd(dppf)Cl₂ (241 mg, 0.33 mmol), and Cs₂CO₃ (3.23 g, 9.92 mmol). The resulting solution was stirred for 14 h at 100° C. in an oil bath. The resulting solution was diluted with 40 mL of water. The resulting solution was extracted with 3×30 mL of DCM and concentrated. The residue was eluted from silica gel with ethyl acetate/petroleum ether (1:5). This resulted in 1.1 g (81.8%) of the title compound as a brown solid. MS-ESI: 204 [M+1].

### Step 3: 4-Isopropyl-2-(trifluoromethyl)pyrimidin-5-amine

Into a 100-mL round-bottom flask purged and maintained with an inert atmosphere of nitrogen, was placed 4-(prop-1-en-2-yl)-2-(trifluoromethyl)pyrimidin-5-amine (1.2 g, 5.91 mmol) in methanol (20 mL), to the stirred solution was added Pd/C (10% wt., 200 mg). The flask was evacuated and filled three times with hydrogen. The resulting solution was stirred 16 h at RT under hydrogen. The solids were filtered out. The resulting mixture was concentrated under vacuum. This resulted in 1.1 g (90.8%) of the title compound as a brown solid. MS-ESI: 206 [M+1].

### Step 4: 4-Bromo-6-isopropyl-2-(trifluoromethyl)pyrimidin-5-amine

Into a 50-mL round-bottom flask purged and maintained with an inert atmosphere of nitrogen, was placed 4-(propan-2-yl)-2-(trifluoromethyl)pyrimidin-5-amine (1.1 g, 5.36 mmol) in acetonitrile (20 mL), to this solution was added NBS (1.15 g, 6.46 mmol) in portions with stirring. The resulting solution was stirred for 12 h at RT. The resulting solution was diluted with 40 mL of water. The resulting solution was extracted with 2×30 mL of DCM concentrated. The residue was eluted from silica gel with ethyl acetate/petroleum ether (1:40 to 1:30). This resulted in 1.0 g (65.6%) of the title compound as a brown solid. MS-ESI: 284/286 [M+1]. Steps 5-9 used similar procedures for converting compound 189″ to intermediate 54 shown in Scheme 37 to afford intermediate 63 from compound 227″. MS-ESI: 291 (M+1).

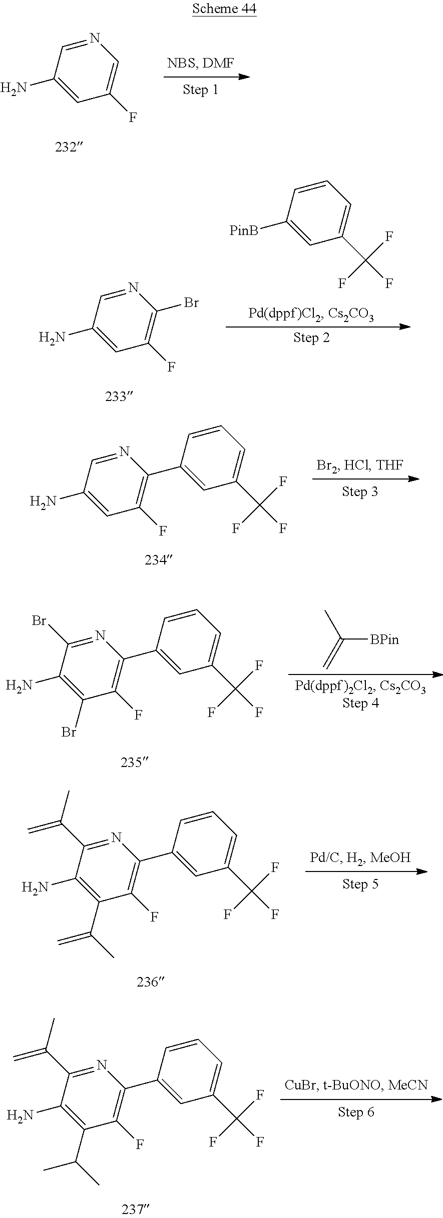

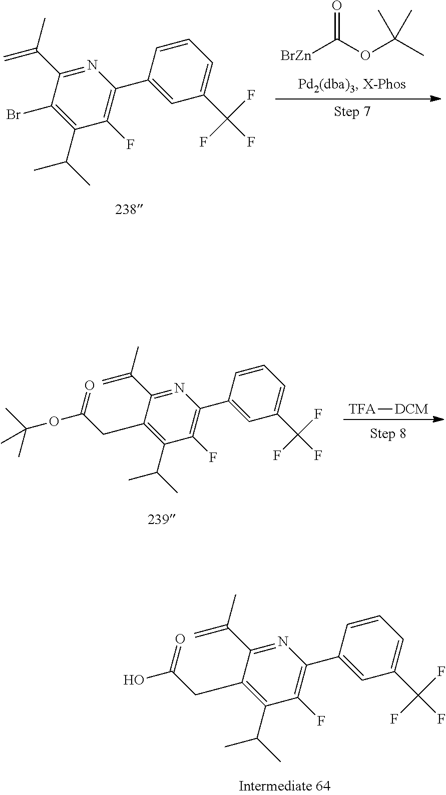

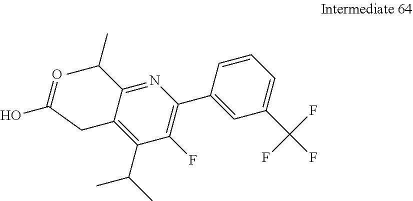 [SMILES: CC(C)c1nc(-c2cccc(C(F)(F)F)c2)c(F)c(C(C)C)c1CC(=O)O]

### 2-(5-Fluoro-2,4-diisopropyl-6-(3-(trifluoromethyl)phenyl)pyridin-3-yl)acetic Acid

### Step 1: 6-Bromo-5-fluoropyridin-3-amine

Into a 100 mL round-bottom flask, was added 5-fluoropyridin-3-amine (2 g, 17.9 mmol) in DMF (15 mL) at RT. To the stirred solution was added NBS (3.19 g, 17.9 mmol) in DMF (5 mL) dropwise at RT. The resulting solution was stirred for 1 h at RT and diluted with water (75 mL). The resulting mixture was extracted with ethyl acetate (3×20 mL). The combined organic layers were dried over anhydrous Na₂SO₄. After filtration, the filtrate was concentrated under reduced pressure. The residue was purified by silica gel chromatography, eluted with EtOAc/petroleum ether (8:1) to afford the title compound (3 g, 79%) as a dark yellow solid. ME-ESI: 191/193 [M+1].

### Step 2: s5-Fluoro-6-(3-(trifluoromethyl)phenyl)pyridin-3-amine

Into a 500-mL round-bottom flask purged and maintained with an inert atmosphere of nitrogen, was added 6-bromo-5-fluoropyridin-3-amine (3 g, 15.7 mmol) in dioxane (200 mL) and H₂O (20 mL) at RT. To the stirred solution were added Pd(dppf)Cl₂ (1.15 g, 1.57 mmol) and Cs₂CO₃ (10.2 g, 31.4 mmol) at RT under nitrogen atmosphere. Then 4,4,5,5-tetramethyl-2-[3-(trifluoromethyl)phenyl]-1,3,2-dioxaborolane (17.1 g, 62.8 mmol) was added to the above mixture. After the addition was complete and the resulting mixture was stirred at 80° C. in an oil bath overnight. The mixture was concentrated and applied into silica gel and eluted with ethyl acetate/petroleum ether (12:1) to afford the title compound (4.1 g, 94.7%) as a yellow oil. MS-ESI: 257 [M+1].

### Step 3: 2,4-Dibromo-5-fluoro-6-(3-(trifluoromethyl)phenyl)pyridin-3-amine

Into a 250 mL round-bottom flask, were added 5-fluoro-6-[3-(trifluoromethyl)phenyl]pyridin-3-amine (4.1 g, 16.0 mmol) in THF (25 mL) at RT. To the stirred solution was added HCl (2 M, 13.5 mL) in one portion at RT. To this mixture was added Br₂ (2.4 mL) dropwise. After the addition was complete, the resulting mixture was stirred for 4 h at RT. The resulting mixture was extracted with ethyl acetate (3×90 mL). The combined organic layers were dried over anhydrous Na₂SO₄. After filtration, the filtrate was concentrated under reduced pressure. The residue was purified by silica gel chromatography, eluted with EtOAc/petroleum ether (20:1) to afford the title compound (2.25 g, 33.9%) as a yellow solid. ME-ESI: 413/415/417 [M+1].

Steps 4-8 used procedures for converting compound 189″ to intermediate 54 shown in Scheme 37 to afford intermediate 64 from compound 235″. MS-ESI: 384 (M+1).

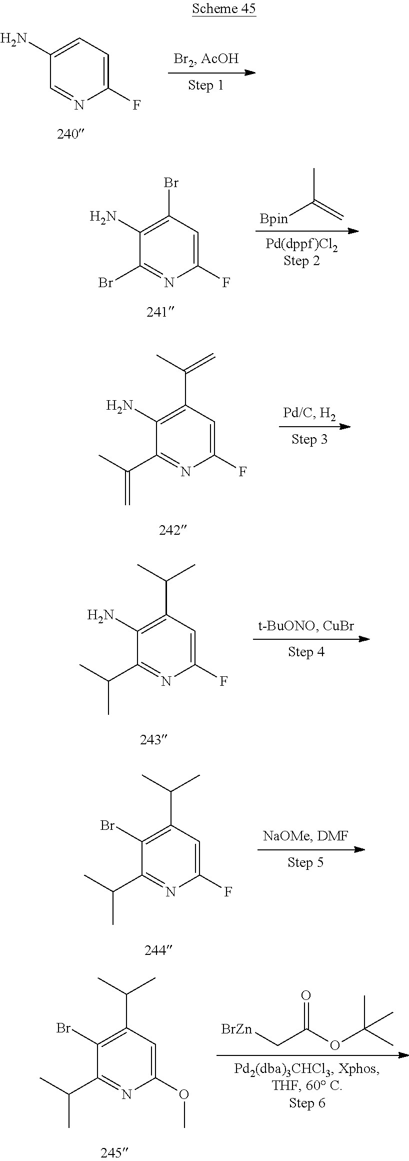

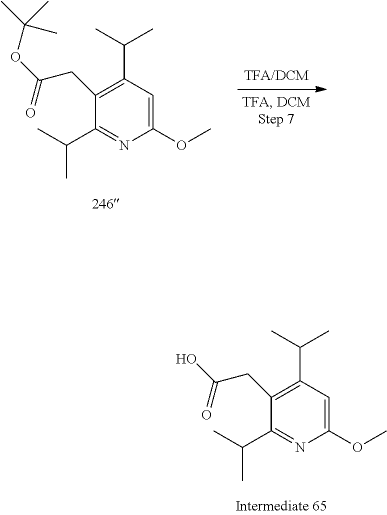 [SMILES: COc1cc(C(C)C)c(CC(=O)OC(C)(C)C)c(C(C)C)n1 ; COc1cc(C(C)C)c(CC(=O)O)c(C(C)C)n1]

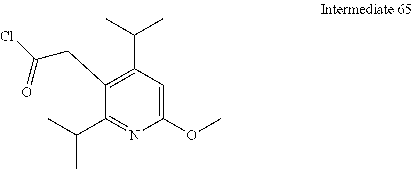 [SMILES: COc1cc(C(C)C)c(CC(=O)Cl)c(C(C)C)n1]

### 2-(2,4-Diisopropyl-6-methoxypyridin-3-yl)acetyl Chloride

### Step 1: 2,4-Dibromo-6-fluoropyridin-3-amine

Into a 1-L round-bottom flask, was placed a solution of 6-fluoropyridin-3-amine (4.05 g, 36.1 mmol) in AcOH (40 mL). This was followed by the addition of a solution of Br₂ (4.1 mL, 79.9 mmol) in AcOH (50 mL) dropwise with stirring at 0° C. The resulting solution was stirred for 16 h at RT. The resulting mixture was washed with 150 ml of saturated solution of NaHCO₃, extracted with 3×200 ml of dichloromethane, and dried over anhydrous sodium sulfate. This resulted in 5 g (51.2%) of the title compound as a yellow solid. MS-ESI: 269/271/273[M+1]. Steps 2-4 used similar procedures for converting compound 189 to compound 192 shown in Scheme 37 to afford compound 244 from compound 241. MS-ESI: 260/262 (M+1).

### Step 5: 3-Bromo-2,4-diisopropyl-6-methoxypyridine

Into a 25-mL round-bottom flask, was placed a solution of 3-bromo-6-fluoro-2,4-bis(propan-2-yl)pyridine (130 mg, 0.50 mmol) in DMF (5 mL). To the solution was added CH₃ONa (108 mg, 2.0 mmol). The resulting solution was stirred overnight at RT. The resulting mixture was washed with 20 ml of H₂O. The resulting solution was extracted with 2×25 ml of ethyl acetate dried over anhydrous sodium sulfate and concentrated. The residue was eluted from silica gel with petroleum ether. This resulted in 100 mg (73.5%) of the title compound as yellow oil. MS-ESI: 272/274 [M+1].

Steps 6-7 used similar procedures for converting compound 192″ to intermediate 54 shown in Scheme 37 to afford intermediate 65 from compound 245″. MS-ESI: 252 (M+1).

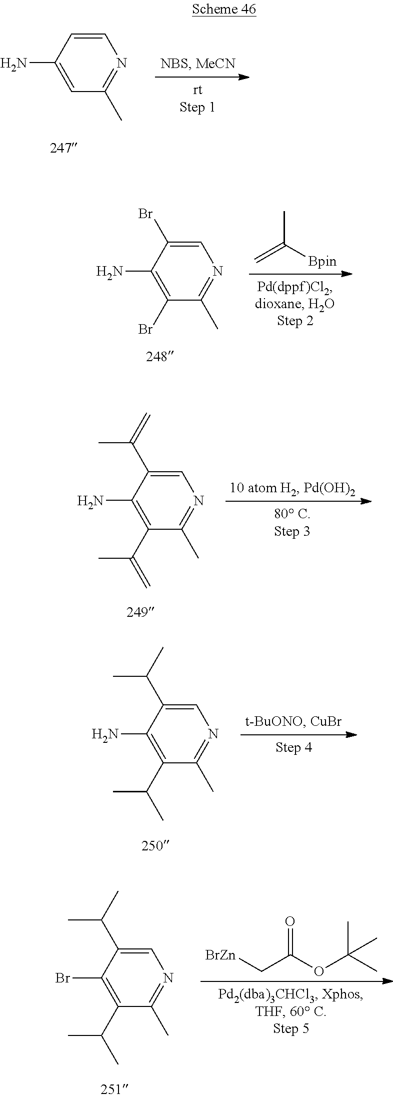

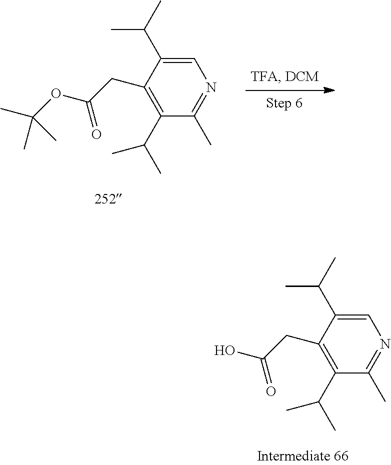 [SMILES: Cc1ncc(C(C)C)c(CC(=O)OC(C)(C)C)c1C(C)C ; Cc1ncc(C(C)C)c(CC(=O)O)c1C(C)C]

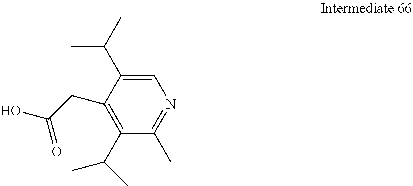 [SMILES: Cc1ncc(C(C)C)c(CC(=O)O)c1C(C)C]

### 2-(3, 5-Diisopropyl-2-methylpyridin-4-yl)acetic Acid

Steps 1-2 used similar procedures for converting compound 161″ to compound 163″ shown in Scheme 31 to afford compound 249″ from compound 247″. MS-ESI: 189 (M+1).

### Step 3: 3, 5-Diisopropyl-2-methylpyridin-4-amine

Into a 1-L pressure tank reactor purged and maintained with an inert atmosphere of nitrogen, was placed 2-methyl-3, 5-bis(prop-1-en-2-yl)pyridin-4-amine (22.0 g, 117 mmol) in MeOH (400 mL). To the stirred solution was added Pd(OH)₂/C (10% wt., 2.0 g). The reaction solution was evacuated and filled three times with hydrogen. The resulting mixture was stirred for 3 days at 80° C. under hydrogen atmosphere. The solids were filtered out. The resulting mixture was concentrated under vacuum. This resulted in 17.5 g (77.7%) of the title compound as brown oil. MS-ESI: 193 [M+1].

Steps 4-6 used similar procedures for converting compound 164″ to Intermediate 36 shown in Scheme 31 to afford intermediate 66 from compound 250″. MS-ESI: 235 (M+1).

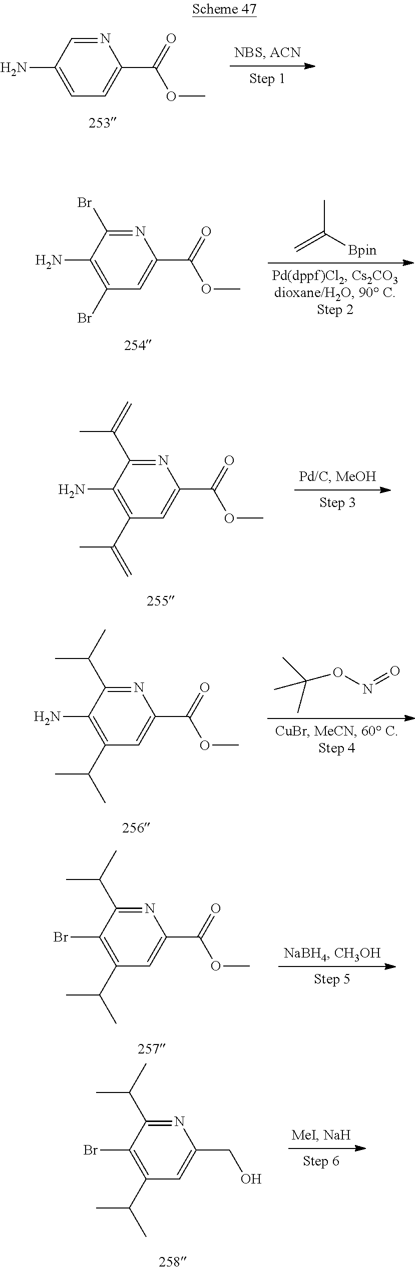

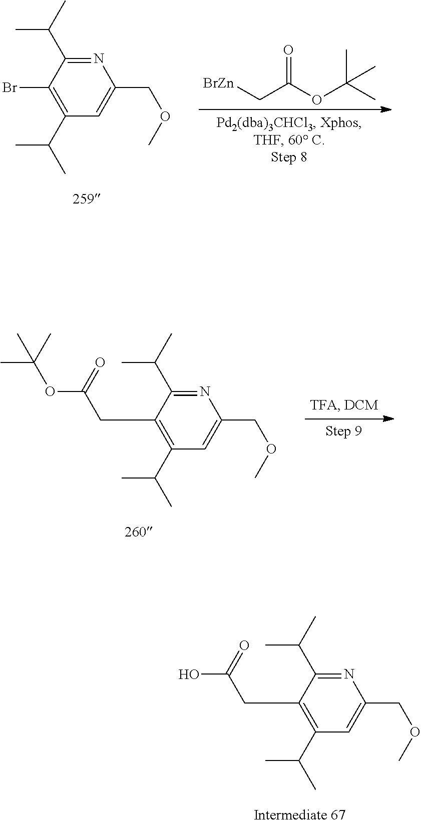

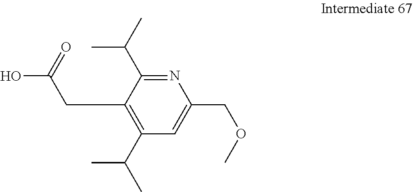 [SMILES: COCc1cc(C(C)C)c(CC(=O)O)c(C(C)C)n1]

### 2-(2,4-Diisopropyl-6-(methoxymethyl)pyridin-3-yl)acetic Acid

Steps 1-4 used similar procedures for converting compound 161″ to compound 165″ shown in Scheme 31 to afford compound 257″ from compound 253″. MS-ESI: 300/302 (M+1).

### Step 5: (5-Bromo-4,6-diisopropylpyridin-2-yl)methanol

Into a 50-mL round-bottom flask, was placed methyl 5-bromo-4,6-bis(propan-2-yl)pyridine-2-carboxylate (1.4 g, 4.66 mmol) in methanol (10 mL). This was followed by the addition of NaBH₄ (532 mg, 13.9 mmol) in several batches at 0° C. The resulting solution was stirred for 1 h at RT. The reaction was then quenched by the addition of 20 mL of water. The resulting solution was extracted with 3×30 mL of ethyl acetate concentrated. This resulted in 1.2 g (94.5%) of the title compound as an off-white solid. MS-ESI: 272/274 [M+1].

### Step 6: 3-Bromo-2,4-diisopropyl-6-(methoxymethyl)pyridine

Into a 100-mL round-bottom flask purged and maintained with an inert atmosphere of nitrogen, was placed [5-bromo-4,6-bis(propan-2-yl)pyridin-2-yl]methanol (800 mg, 2.94 mmol) in THF (10 mL). This was followed by the addition of NaH (60% wt. oil dispersion, 353 mg, 8.82 mmol) in several batches at 0° C. The resulting solution was stirred for 20 min at RT. To this was added CH₃I (1.25 g, 8.82 mmol) dropwise with stirring at 0° C. The resulting solution was allowed to react, with stirring, overnight at RT. The reaction was then quenched by the addition of 20 mL of water. The resulting solution was extracted with 3×30 mL of dichloromethane and concentrated. The residue was eluted from silica gel with ethyl acetate/petroleum ether (1:10). This resulted in 820 mg (97.4%) of the title compound as light yellow oil.

Steps 8-9 used similar procedures for converting compound 165″ to intermediate 36 shown in Scheme 31 to afford intermediate 67 from compound 259″. MS-ESI: 286/287 (M+1).

Schemes for Sulfonimidoylamide Intermediates: Schemes 47-56 illustrate the preparation of sulfonimidoylamide intermediates.

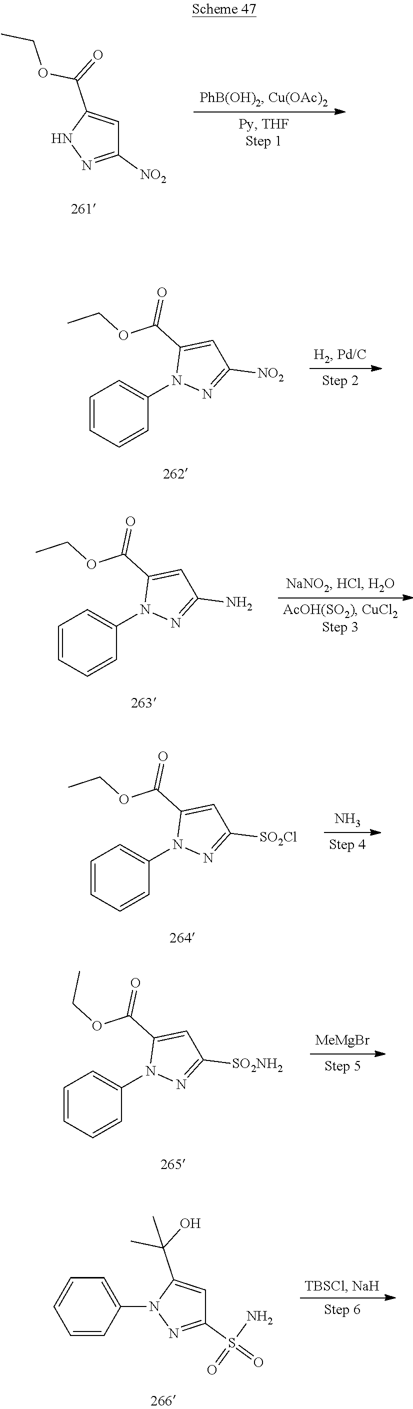

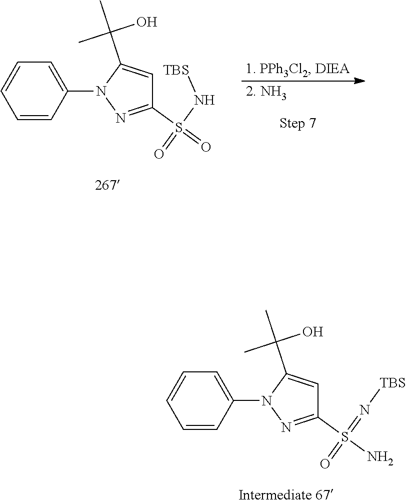

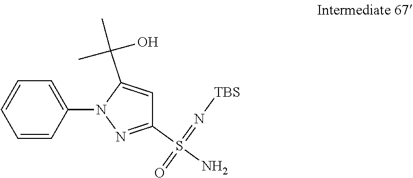

### N′-(tert-butyldimethylsilyl)-5-(2-hydroxypropan-2-yl)-1-phenyl-1H-pyrazole-3-sulfonimidamide

### Step 1: Ethyl 3-nitro-1-phenyl-1H-pyrazole-5-carboxylate

To a stirred solution of ethyl 3-nitro-1H-pyrazole-5-carboxylate (5.0 g, 27.0 mmol) in THF (150 mL) in a 250-mL round-bottom flask was added phenylboronic acid (6.6 g, 54.1 mmol), Cu(OAc)₂ (7.38 g, 40.6 mmol), and pyridine (8.54 g, 108 mmol). The resulting solution was stirred overnight at RT under air. The insoluble matter was filtered out and the filtrate was concentrated under vacuum. The residue was eluted from a silica gel column with EtOAc/PE (1:1). This resulted in 3.1 g (44%) of the title compound as an off-white solid. MS-ESI: 262 (M+1).

### Step 2: Ethyl 3-amino-1-phenyl-1H-pyrazole-5-carboxylate

To a stirred solution of ethyl 3-nitro-1-phenyl-1H-pyrazole-5-carboxylate (3.92 g, 15.0 mmol) in MeOH (50 mL) in a 100-mL round-bottom flask was added Pd/C (wet 10% wt., 400 mg). The flask was evacuated and filled three times with hydrogen. The solution was stirred for 16 h at RT under hydrogen with a balloon. The solids were filtered out. The filtrate was concentrated under vacuum. This resulted in 2.8 g (81%) of the title compound as a light yellow solid. MS-ESI: 232 (M+1).

### Step 3: Ethyl 3-(chlorosulfonyl)-1-phenyl-1H-pyrazole-5-carboxylate

To a stirred solution of ethyl 3-amino-1-phenyl-1H-pyrazole-5-carboxylate (1.8 g, 7.78 mmol) in HCl (6.0 M, 15 mL) in a 100-mL round-bottom flask was added a solution of NaNO₂ (646 mg, 9.36 mmol) in water (2 mL) dropwise at 0° C. The resulting solution was stirred for 30 min at 0° C., this solution was assigned as solution A. CuCl₂ (1.05 g, 7.81 mmol) was added to AcOH (20 mL) in a 250-mL round-bottom flask, SO₂ (g) was bubbled to the solution with stirring at RT for 20 min, this solution was assigned as solution B. To the solution B was added solution A dropwise with stirring at 0° C. The resulting solution was stirred for 2 h at RT. The reaction was then quenched by the addition of 50 mL of water. The resulting solution was extracted with 3×100 mL of DCM and the organic layers were combined, dried over anhydrous Na₂SO₄ and concentrated under vacuum. This resulted in 2.2 g (crude) of the title compound as a light yellow solid. The reaction was monitored by the addition of MeOH, gave MS-ESI: 311 (M+1).

### Step 4: Ethyl 1-phenyl-3-sulfamoyl-1H-pyrazole-5-carboxylate

To a stirred solution of ethyl 3-(chlorosulfonyl)-1-phenyl-1H-pyrazole-5-carboxylate (2.2 g, crude) in DCM (30 mL) in a 100-mL round-bottom flask was bubbled NH₃ gas at 0° C. for 10 min. The resulting solution was stirred for 2 h at RT. The resulting mixture was concentrated under vacuum. The residue was eluted from a silica gel column with EtOAc/PE (1:1). This resulted in 1.07 g (46.5% over 2 steps) of the title compound as a light yellow solid. MS-ESI: 296 (M+1).

### Step 5: 5-(2-Hydroxypropan-2-yl)-1-phenyl-1H-pyrazole-3-sulfonamide

To a stirred solution of ethyl 1-phenyl-3-sulfamoyl-1H-pyrazole-5-carboxylate (1.65 g, 5.59 mmol) in THF (30 mL) in a 100-mL 3-necked round-bottom flask under nitrogen was added MeMgBr/THF (3.0 M, 18.6 mL, 55.8 mmol) dropwise at 0° C. The resulting solution was stirred overnight at RT. The reaction was then quenched by the addition of 30 mL of NH₄Cl (sat.). The resulting solution was extracted with 3×100 mL of DCM and the organic layers were combined, dried over anhydrous Na₂SO₄ and concentrated under vacuum. The residue was eluted from a silica gel column with EtOAc/PE (2:1). This resulted in 1.35 g (86%) of the title compound as a yellow solid. MS-ESI: 282 (M+1).

### Step 6: N-(tert-butyldimethylsilyl)-5-(2-hydroxypropan-2-yl)-1-phenyl-1H-pyrazole-3-sulfonamide

To a stirred solution of 5-(2-hydroxypropan-2-yl)-1-phenyl-1H-pyrazole-3-sulfonamide (500 mg, 1.78 mmol) in THF (10 mL) in a 100-mL round-bottom flask was added NaH (60% wt. oil dispersion, 86 mg, 3.58 mmol) in portions at 0° C. Then to the above was added TBSCl (538 mg, 3.57 mmol). The resulting solution was stirred for 2 h at RT. The reaction was then quenched by the addition of 10 mL of water. The resulting solution was extracted with 3×20 mL of DCM, the organic layers were combined and dried over anhydrous Na₂SO₄ and concentrated under vacuum. The residue was eluted from a silica gel with EtOAc/PE (1:2). This resulted in 660 mg (94%) of the title compound as a light yellow solid. MS-ESI: 396 (M+1).

### Step 7: N′-(tert-butyldimethylsilyl)-5-(2-hydroxypropan-2-yl)-1-phenyl-1H-pyrazole-3-sulfonimidamide

To a stirred solution of PPh₃Cl₂ (1.67 g, 5.01 mmol) in CHCl₃ (30 mL) in a 100-mL 3-necked round-bottom flask under nitrogen was added DIEA (1.29 g, 9.98 mmol) dropwise at RT. The resulting solution was stirred for 10 min at RT and the reaction system was cooled to 0° C. To this was added a solution of N-(tert-butyldimethylsilyl)-5-(2-hydroxypropan-2-yl)-1-phenyl-1H-pyrazole-3-sulfonamide (660 mg, 1.67 mmol) in CHCl₃ (3 mL) dropwise with stirring at 0° C. The resulting solution was stirred for 30 min at 0° C. Then NH₃ gas was bubbled into the mixture for 15 min at 0° C. The resulting solution was stirred for 2 h at RT. The resulting solution was diluted with 30 mL of water. The resulting solution was extracted with 3×100 mL of DCM, the organic layers were combined and dried over anhydrous Na₂SO₄ and concentrated under vacuum. The residue was eluted from silica gel with EtOAc/PE (1:1). This resulted in 530 mg (81%) of the title compound as a light yellow solid. MS-ESI: 395 (M+1).

TABLE 23

| | | | Exact |
|---|---|---|---|
| | | | Mass |
| Intermediate # | Structure | IUPAC Name | [M − H]⁻ |
| Intermediate 68′ | 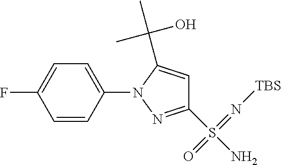 | N′-(tert-butyldimethylsilyl)-1-(4-fluorophenyl)-5-(2- hydroxypropan-2-yl)-1H-pyrazole-3-sulfonamide | 413 |

The Intermediates in the following Table were prepared using the similar procedures for converting compound 261′ to Intermediate 67′ shown in Scheme 47 from appropriated starting materials.

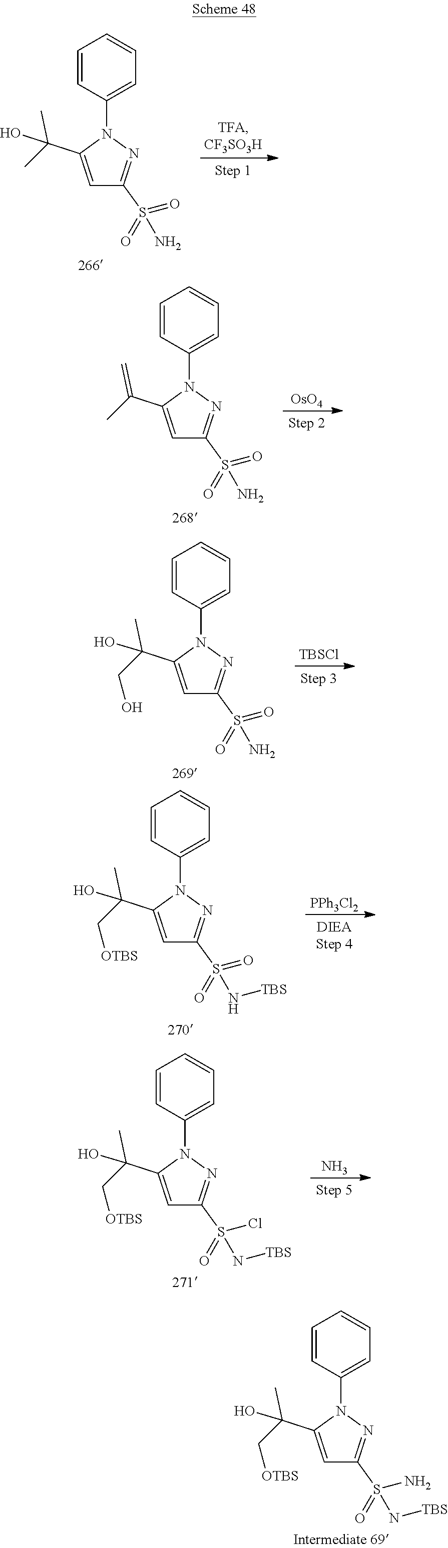

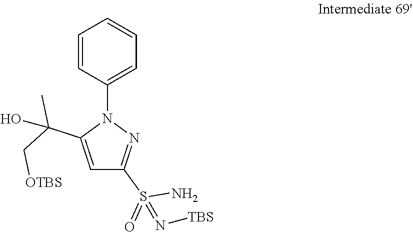

### N′-(tert-butyldimethylsilyl)-5-(1-((tert-butyldimethylsilyl)oxy)-2-hydroxypropan-2-yl)-1-phenyl-1H-pyrazole-3-sulfonimidamide

### Step 1: 1-Phenyl-5-(prop-1-en-2-yl)-1H-pyrazole-3-sulfonamide

To a stirred solution of 5-(2-hydroxypropan-2-yl)-1-phenyl-1H-pyrazole-3-sulfonamide (5.80 g, 20.6 mmol) in TFA (12 mL) in a 100-mL round-bottom flask under nitrogen was added CF₃SO₃H (12 mL). The resulting solution was stirred for 6 h at RT. The pH value of the solution was adjusted to 8 with a solution of NaOH (3% wt., aq.). The resulting solution was extracted with 3×500 mL of DCM and the organic layers were combined and concentrated under vacuum. The residue was eluted from a silica gel with EtOAc/PE (1:1). This resulted in 4.8 g (88.7%) of the title compound as a light yellow crude solid. MS-ESI: 264 (M+1).

### Step 2: 5-(1,2-Dihydroxypropan-2-yl)-1-phenyl-1H-pyrazole-3-sulfonamide

To a stirred solution of 1-phenyl-5-(prop-1-en-2-yl)-1H-pyrazole-3-sulfonamide (1.0 g, 3.8 mmol) in t-BuOH (4.0 mL) and acetone (4.0 mL) in a 100-mL round-bottom flask under nitrogen was added NMO (890 mg, 7.6 mmol). The resulting solution was stirred for 15 min at RT. Then a solution of OsO₄ (99 mg, 0.39 mmol) in H₂O (2 mL) was added dropwise to the stirred solution. The resulting solution was stirred for 16 h at RT. The reaction was then quenched by the addition of 5.0 mL of sat. Na₂S₂O₃. The resulting solution was extracted with 3×100 mL of EtOAc and the organic layers were combined and dried over anhydrous Na₂SO₄. The crude product was eluted from a silica gel with MeOH/DCM (7:100). This resulted in 1.1 g (97.4%) of the title compound as a light yellow crude solid. MS-ESI: 298 (M+1).

Steps 3-5 used similar procedures for converting compound 266′ to Intermediate 67′ shown in Scheme 47 to afford Intermediate 69′ from compound 269′. MS-ESI: 525 (M+1).

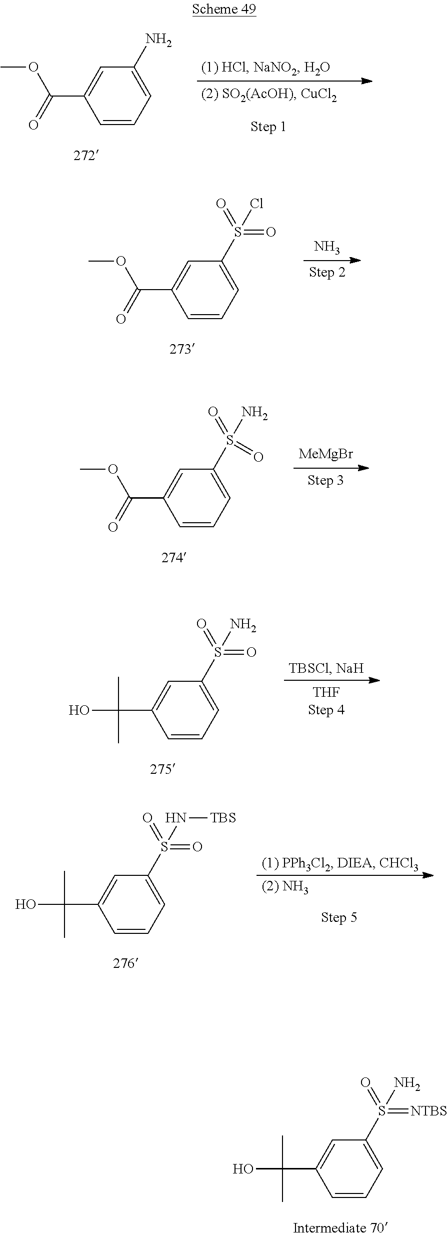

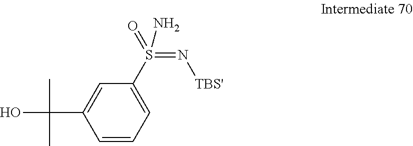

### N′-(tert-butyldimethylsilyl)-3-(2-hydroxypropan-2-yl)benzenesulfonimidamide

Steps 1-5 used similar procedures for converting compound 263′ to Intermediate 67′ shown in Scheme 47 to afford Intermediate 70′ from compound 272′. MS-ESI: 329 (M+1).

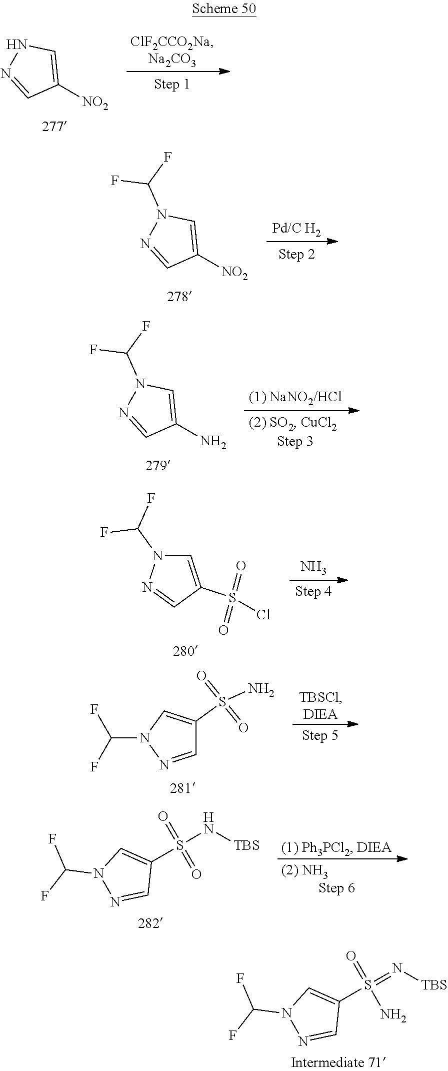

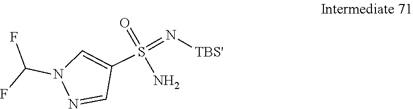

### N′-(tert-butyldimethylsilyl)-1-(difluoromethyl)-1H-pyrazole-4-sulfonimidamide

### Step 1: 1-(Difluoromethyl)-4-nitro-1H-pyrazole

To a stirred solution of 4-nitro-1H-pyrazole (20 g, 177 mmol) in DMF (150 mL) in a 500-mL round-bottom flask was added Na₂CO₃ (28.1 g, 265 mmol) and sodium 2-chloro-2,2-difluoroacetate (32.4 g, 212 mmol). The resulting solution was stirred for 4 h at 90° C. in an oil bath. The reaction was then quenched by the addition of 150 mL of water. The resulting solution was extracted with 2×200 mL of EtOAc. The combined organic layer was washed with 2×100 ml of H₂O, dried over anhydrous Na₂SO₄ and concentrated under vacuum. This resulted in 26 g (94.7%) of the title compound as a brown liquid. MS-ESI: 164 (M+1).

Steps 2-4 used similar procedures for converting compound 262′ to compound 265′ shown in Scheme 47 to afford compound 281′ from compound 278′. MS-ESI: 198 (M+1).

Steps 5-6 used similar procedures for converting compound 266′ to intermediate 67′ shown in Scheme 47 to afford Intermediate 71′ from compound 281′. MS-ESI: 311 (M+1).

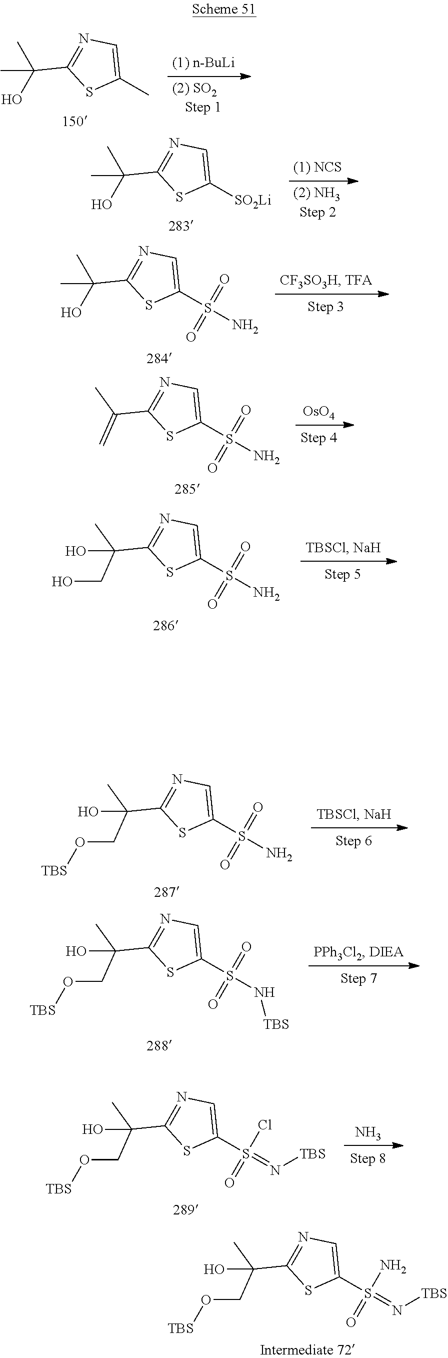

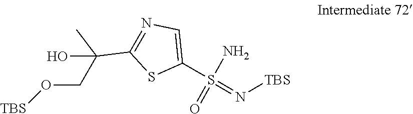

### N′-(tert-butyldimethylsilyl)-2-(1-((tert-butyldimethylsilyl)oxy)-2-hydroxypropan-2-yl)thiazole-5-sulfonimidamide

### Step 1: Lithium 2-(2-hydroxypropan-2-yl)thiazole-5-sulfinate

To a stirred solution of 2-(thiazol-2-yl)propan-2-ol (20 g, 140 mmol) in THF (400 mL) in a 1-L 3-necked round-bottom flask under nitrogen was added n-BuLi (2.50 M, 140 mL, 350 mmol) dropwise at −78° C. Then the resulting solution was stirred at −78° C. for 1 h. Then SO₂ (g) was bubbled to the solution at −50° C. for 20 min. The resulting solution was allowed to react, with stirring, for an additional 2 h at RT. The resulting mixture was concentrated under vacuum. This resulted in 20 g (crude) of the title compound as a yellow crude solid. MS-ESI. 206 (M−1).

### Step 2: 2-(2-Hydroxypropan-2-yl)thiazole-5-sulfonamide

To a stirred solution of lithium 2-(2-hydroxypropan-2-yl)-1,3-thiazole-5-sulfinate (20 g, 93.8 mmol) in DCM (400 mL) in a 1-L round-bottom flask was added NCS (18.8 g, 141 mmol) in portions at 0° C. The resulting solution was stirred for 2 h at RT. The resulting solution was diluted with 500 ml of water, then extracted with 3×500 mL of DCM and the organic layers were combined and dried over anhydrous Na₂SO₄. Then NH₃ (g) was bubbled into the reaction mixture for 30 min at 0° C. The resulting solution was stirred for 2 h at RT. The resulting mixture was concentrated under vacuum. The residue was eluted from silica gel with EtOAc/PE (1/1). This resulted in 2 g (6.43% over two steps) of the title compound as a brown solid. MS-ESI: 223 (M+1).

Steps 3-8 used similar procedures for converting compound 266′ to intermediate 69′ shown in Scheme 48 to afford intermediate 72′ from compound 284′. MS-ESI: 466 (M+1).

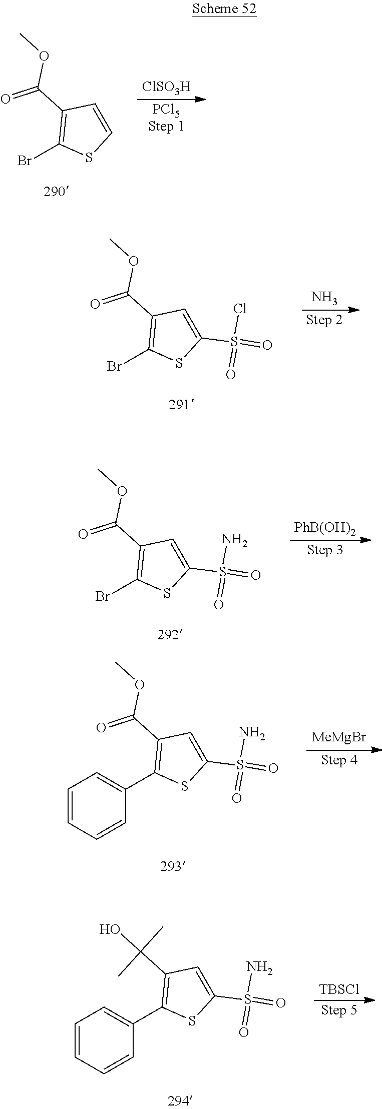

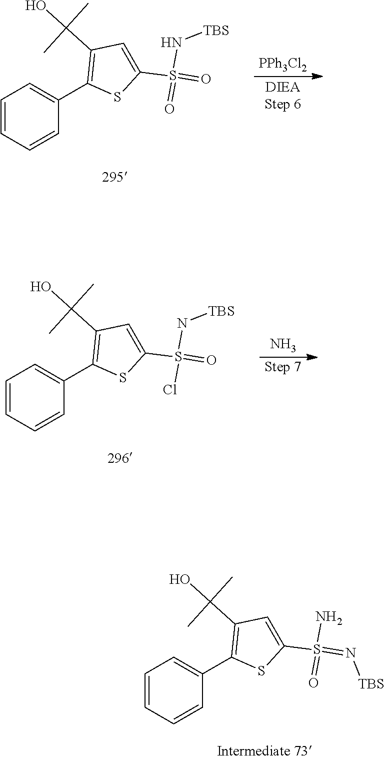

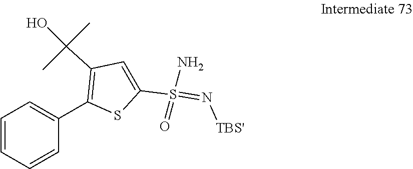

### N′-(tert-butyldimethylsilyl)-4-(2-hydroxypropan-2-yl)-5-phenylthiophene-2-sulfonimidamide

### Step 1: Methyl 2-bromo-5-(chlorosulfonyl)thiophene-3-carboxylate

To a stirred solution of methyl 2-bromothiophene-3-carboxylate (4.42 g, 20.0 mmol) in CHCl₃ (100 mL) in a 250-mL round-bottom flask was added ClSO₃H (7.02 g, 60.0 mmol) dropwise at 0° C. The resulting solution was stirred for 16 h at RT. This was followed by the addition of PCl₅ (12.5 g, 60.0 mmol) in several batches at 0° C. The resulting solution was stirred for 3 h at 60° C. in an oil bath. The reaction mixture was poured into 200 mL of water/ice. The resulting solution was extracted with 3×100 mL of DCM, the combined organic layers were dried over Na₂SO₄ then concentrated under vacuum. This resulted in 4.50 g (crude) title compound as light yellow oil.

### Step 2: Methyl 2-bromo-5-sulfamoylthiophene-3-carboxylate

To a stirred solution of methyl 2-bromo-5-(chlorosulfonyl)thiophene-3-carboxylate (4.50 g, crude) in DCM (100 mL) in a 250-mL round-bottom flask was bubbled NH₃ (g) with stirring at 0° C. for 10 min. The resulting solution was stirred for 2 h at RT, the mixture was concentrated under vacuum. The crude product was eluted from silica gel with EtOAc/PE (1:1). This resulted in 3.04 g (50.6%, over two steps) of the title compound as yellow solid. MS-ESI: 300/298 (M−1).

### Step 3: Methyl 2-phenyl-5-sulfamoylthiophene-3-carboxylate

To a stirred solution of methyl 2-bromo-5-sulfamoylthiophene-3-carboxylate (3.0 g, 10.0 mmol) in dioxane (100 mL)/H₂O (10 mL) in a 500-mL 3-necked round-bottom flask under nitrogen was added phenylboronic acid (3.05 g, 25.0 mmol). Then Cs₂CO₃ (8.15 g, 25.0 mmol), Xphos (477 mg, 1.0 mmol) and Pd(dppf)Cl₂ (732 mg, 1.0 mmol) were added. The resulting solution was stirred for 16 h at 80° C. in an oil bath. The insoluble was filtered out and the filtrate was concentrated under vacuum. The residue was eluted from silica gel with EtOAc/PE (1:3). This resulted in 1.81 g (61.1%) of the title compound as yellow oil. MS-ESI: 296 (M−1).

Steps 4-7 used similar procedures for converting compound 265′ to intermediate 67′ shown in Scheme 47 to afford intermediate 73′ from compound 293′. MS-ESI: 411 (M+1).

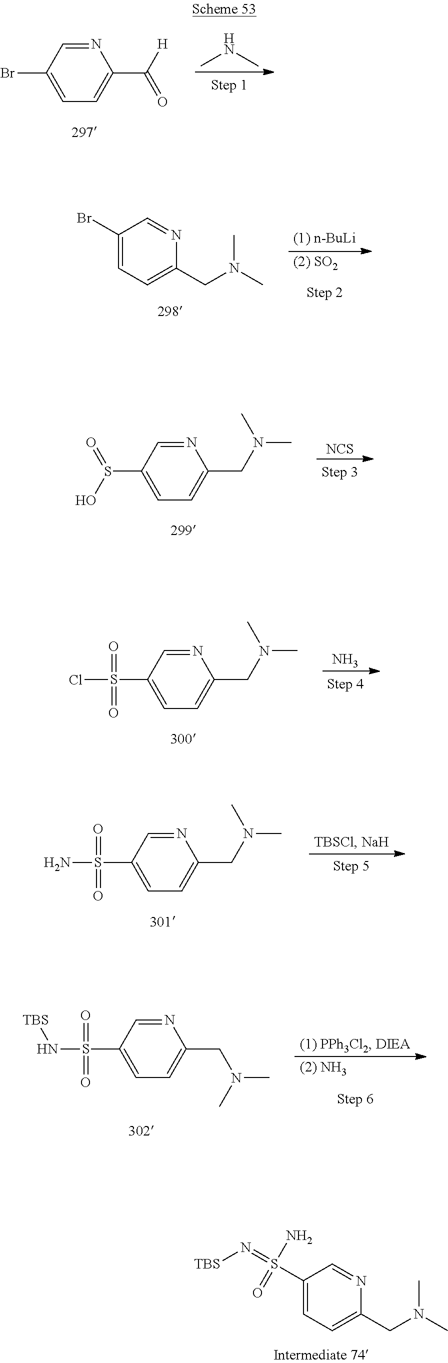

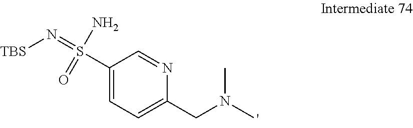

### N′-(tert-butyldimethylsilyl)-5-((dimethylamino)methyl)pyridine-2-sulfonimidamide

Steps 1-6 used similar procedures for converting compound 143′ to Intermediate 33′ shown in Scheme 28 to afford Intermediate 74′ from compound 297′. MS-ESI: 216 (M+1).

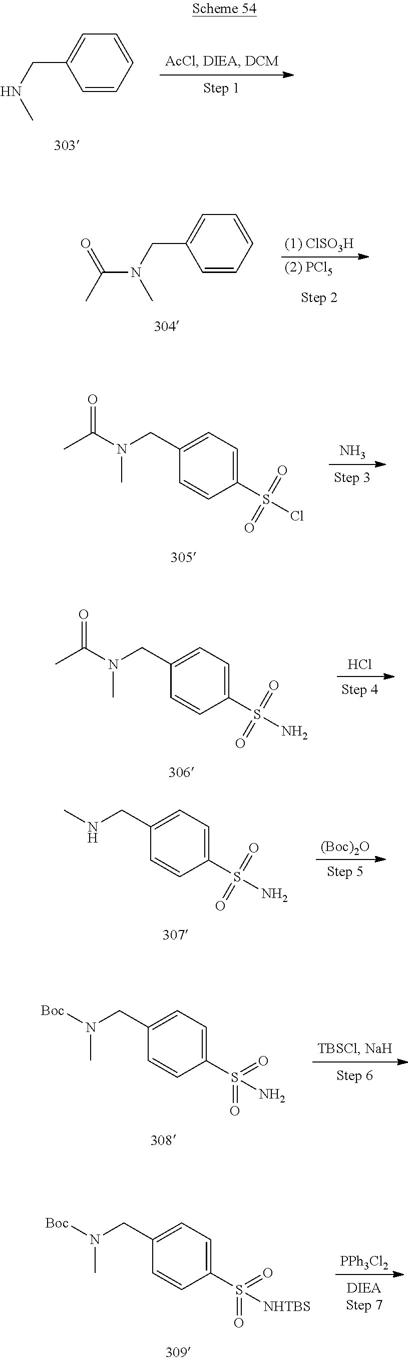

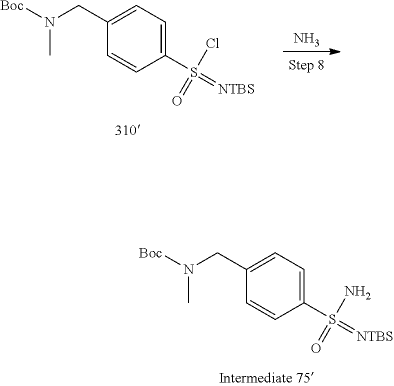

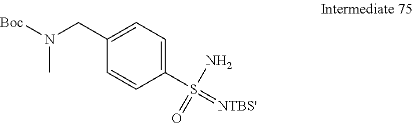

### Tert-butyl 4-(N′-(tert-butyldimethylsilyl)sulfamimidoyl)benzyl(methyl)carbamate

### Step 1: N-benzyl-N-methyl Acetamide

To a stirred solution of benzyl(methyl)amine (10 g, 82.5 mmol) in DCM (500 mL) and DIEA (21.3 g, 165 mmol) in a 1000 mL round-bottom flask was added acetyl chloride (9.72 g, 124 mmol) dropwise at 0° C. The resulting mixture was stirred for 4 h at RT. The resulting mixture was concentrated under reduced pressure. The residue was eluted from silica gel column with EtOAc/PE (1:1) to afford the title compound (13 g, 96.5%) as yellow oil. MS-ESI: 164 (M+1).

Steps 2-3 used similar procedures for converting compound 290′ to compound 292′ shown in Scheme 52 to afford compound 306′ from compound 304′. MS-ESI: 243 (M+1).

### Step 4: 4-((Methylamino)methyl)benzenesulfonamide

To a stirred solution of N-methyl-N-[(4-sulfamoylphenyl)methyl]acetamide (5.0 g, 20.6 mmol) in HCl (8 M, 200 mL) in a 500-mL round-bottom flask. The resulting solution was stirred for 16 h at 100° C. in an oil bath. The pH value of the solution was adjusted to 8 with the solution of NaOH (3% wt. aq.). The resulting solution was extracted with 3×300 mL of EtOAc and the organic layers were combined, dried over Na₂SO₄ and concentrated under vacuum. The residue was eluted from silica gel with EtOAc/PE (1:1). This resulted in 3.9 g (94%) of the title compound as an off-white solid. MS-ESI: 201 (M+1).

### Step 5: Tert-butyl methyl(4-sulfamoylbenzyl)carbamate

To a stirred solution of 4-[(methylamino)methyl]benzene-1-sulfonamide (5.0 g, 25 mmol) in DCM (100 mL) in a 250-mL round-bottom flask was added DIEA (6.45 g, 50 mmol). Then DMAP (305 mg, 2.5 mmol) and di-tert-butyl dicarbonate (6.0 g, 27.5 mmol) were added in portions at 0° C. The resulting solution was stirred for 5 h at RT. The reaction was quenched with water (100 mL). The resulting solution was extracted with 3×200 mL of EtOAc and the organic layers were combined, dried over Na₂SO₄ and concentrated under vacuum. The residue was eluted from silica gel with EtOAc/PE (1:1). This resulted in 5.0 g (66.7%) of the title compound as a light yellow solid. MS-ESI: 301 (M+1).

Steps 6-8 used similar procedures for converting compound 266′ to intermediate 67′ shown in Scheme 47 to afford Intermediate 75′ from compound 308′. MS-ESI: 414 (M+1).

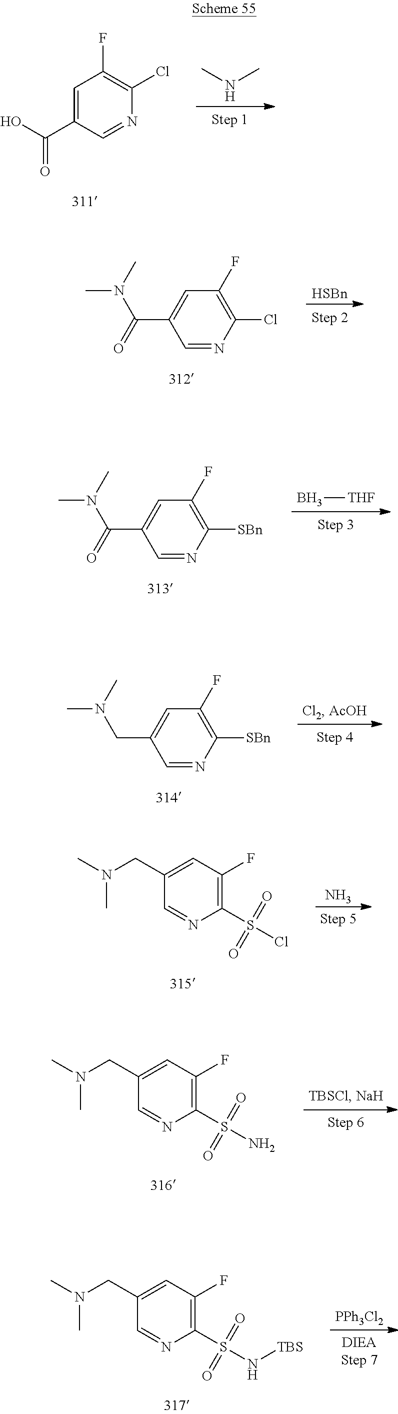

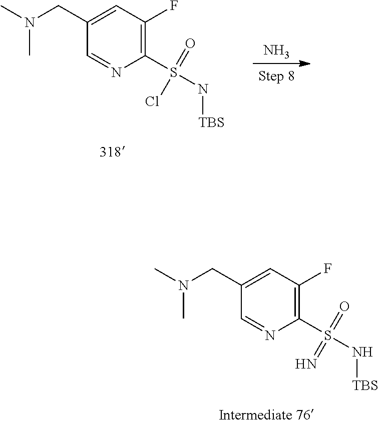

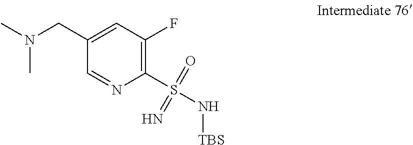

### N-(tert-butyldimethylsilyl)-5-((dimethylamino)methyl)-3-fluoropyridine-2-sulfonimidamide

### Step 1: 6-Chloro-5-fluoro-N,N-dimethylnicotinamide

To a stirred solution of 6-chloro-5-fluoronicotinic acid (10 g, 49.5 mmol) in THF (150 mL) in a 250-mL round-bottom flask under nitrogen was added HATU (28.2 g, 74.3 mmol), DIEA (12.8 g, 99 mmol) and dimethylamine in THF (2 M, 75 mL, 150 mmol) at RT. The resulting solution was stirred for 16 h at RT. The reaction was quenched with water (400 mL). The resulting solution was extracted with 3×400 mL of EtOAc and the organic layers were combined, dried over Na₂SO₄ and concentrated under vacuum. The residue was eluted from a silica gel column with EtOAc/PE (1:1). This resulted in 8.0 g (80%) of the title compound as a white solid. MS-ESI: 203/205 (M+1).

### Step 2: 6-(Benzylthio)-5-fluoro-N,N-dimethylnicotinamide

To a stirred solution of 6-chloro-5-fluoro-N,N-dimethylnicotinamide (8.0 g, 39.4 mmol) in dioxane (160 mL) in a 100-mL round-bottom flask under nitrogen was added phenylmethanethiol (9.76 g, 78.8 mmol) and t-BuOK (8.84 g, 78.8 mmol). The resulting solution was stirred for 16 h at 80° C. The resulting mixture was quenched with water (400 mL), extracted with 3×500 mL of EtOAc and the organic layers were combined and dried over anhydrous Na₂SO₄. The solids were filtered out. The resulting mixture was concentrated under vacuum. The residue was eluted from silica gel with EtOAc/PE (1:1). This resulted in 6.0 g (52%) of the title compound as yellow oil. MS-ESI: 291 (M+1).

### Step 3: 1-(6-(Benzylthio)-5-fluoropyridin-3-yl)-N,N-dimethylmethanamine

To a stirred solution of 6-(benzylthio)-5-fluoro-N,N-dimethylnicotinamide (6.0 g, 20.7 mmol) in THF (100 mL) in a 250-mL round-bottom flask under nitrogen was added BH₃ in THF (1 M, 104 mmol, 104 mL) dropwise at 0° C. The resulting solution was stirred for 16 h at RT. The reaction was quenched with MeOH (100 mL). The resulting mixture was concentrated under vacuum. The residue was eluted from silica gel with EtOAc/PE (1:1). This resulted in 4.4 g (77.1%) of the title compound as a white solid. MS-ESI: 277 (M+1).

### Step 4: 5-((Dimethylamino)methyl)-3-fluoropyridine-2-sulfonyl Chloride

To a stirred solution of 1-(6-(benzylthio)-5-fluoropyridin-3-yl)-N,N-dimethylmethanamine (4.4 g, 15.9 mmol) in DCM (30 mL) in a 250-mL 3-necked round-bottom flask under nitrogen was added AcOH (15 mL) and H₂O (15 mL). Then Cl₂ gas was bubbled into this mixture at 0° C. for 30 min. Then the resulting solution was stirred for another 30 min at 0° C. The resulting mixture was diluted with water (100 mL) and extracted with 3×100 mL DCM. The organic layers were combined and dried over anhydrous Na₂SO₄. This resulted in the title compound in DCM solution used for next step directly.

### Step 5: 5-((Dimethylamino)methyl)-3-fluoropyridine-2-sulfonamide

To a stirred solution of 5-((dimethylamino)methyl)-3-fluoropyridine-2-sulfonyl chloride (crude) in DCM (330 mL) was introduced NH₃ gas bubbled for 30 min at 0° C. The resulting solution was stirred for 2 h at RT. The resulting mixture was concentrated under vacuum. The residue was eluted from silica gel with EtOAc/PE (1:1). This resulted in 1.56 g (42% over two steps) of the title compound as a white solid. MS-ESI: 234 (M+1).

Steps 6-8 used similar procedures for converting compound 266′ to intermediate 67′ shown in Scheme 47 to afford Intermediate 76′ from compound 316′. MS-ESI: 346 (M+1).

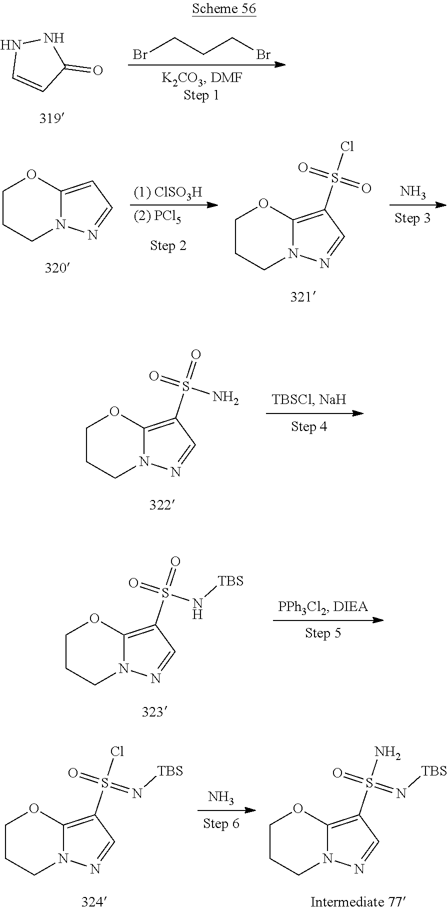

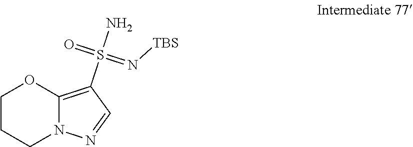

### N′-(tert-butyldimethylsilyl)-6,7-dihydro-5H-pyrazolo[5,1-b][1,3]oxazine-3-sulfonimidamide

### Step 1: 6,7-Dihydro-5H-pyrazolo[5,1-b][1,3]oxazine

To a stirred solution of 1,2-dihydro-3H-pyrazol-3-one (42 g, 500 mmol) in DMF (500 mL) was added K₂CO₃ (138 g, 1.0 mol) in portions at RT, 1,3-dibromopropane (111 g, 550 mmol) was added dropwise at RT. The resulting mixture was stirred for 16 h at 130° C. under nitrogen. The insoluble matter was filtered out, the filtrate was poured into 1500 mL of water, extracted with 3×500 mL of EtOAc. The organic layers were combined, dried over anhydrous Na₂SO₄ and concentrated under reduced pressure. The residue was eluted from silica gel with PE/EtOAc (20:1) to afford 24.8 g (40%) the title compound as a yellow solid. MS-ESI: 125 (M+1). ¹H NMR (400 MHz, CDCl₃): δ 7.31 (d, J=2.0 Hz, 1H), 5.48 (d, J=2.0 Hz, 1H), 4.28 (t, J=5.2 Hz, 2H), 4.18 (t, J=6.2 Hz, 2H), 2.30-2.23 (m, 2H)

### Step 2: 6,7-Dihydro-5H-pyrazolo[5,1-b][1,3]oxazine-3-sulfonyl Chloride

To a stirred solution of 6,7-dihydro-5H-pyrazolo[5,1-b][1,3]oxazine (24 g, 194 mmol) in chlorosulfonic acid (143 mL) in a 1000-mL 3-necked round-bottom flask under nitrogen. The resulting solution was stirred for 16 h at 80° C. The reaction mixture was poured into 1500 mL of water/ice very slowly, extracted with 3×500 mL of EtOAc. The organic layers were combined, dried over anhydrous Na₂SO₄ and concentrated under reduced pressure. The residue was washed with 300 mL of petroleum. This resulted in 28.1 g (65.0%) of the title compound as a yellow solid. MS-ESI: 223/225 (M+1).

### Step 3: 6,7-Dihydro-5H-pyrazolo[5,1-b][1,3]oxazine-3-sulfonamide

To a stirred solution of ammonia (40 mL) in a 1000-mL 3-necked round-bottom flask under nitrogen was added 6,7-dihydro-5H-pyrazolo[5,1-b][1,3]oxazine-3-sulfonyl chloride (28 g, 126 mmol) in THF (80 mL) dropwise at RT. The resulting solution was stirred for 16 h at 60° C. The organic solvent was removed under reduced pressure, then the residue was extracted with 3×500 mL of EtOAc. The organic layers were combined, dried over anhydrous Na₂SO₄ and concentrated under reduced pressure. The residue was purified by silica gel chromatography, eluted with PE/EtOAc (1:1). This resulted in 16.9 g (66.0%) of the title compound as a light yellow solid. MS-ESI: 202 (M−1). ¹H NMR (300 MHZ, DMSO-d₆): δ 7.47 (s, 1H), 7.08 (s, 2H), 4.40 (t, J=5.1 Hz, 2H), 4.10 (t, J=6.0 Hz, 2H), 2.25-2.15 (m, 2H).

Steps 4-6 used similar procedures for converting compound 266′ to intermediate 67′ shown in Scheme 47 to afford Intermediate 77′ from compound 322′. MS-ESI: 317 (M+1).

Schemes for phenylacetic acids Intermediates: Schemes 57-67 illustrate the preparation of phenylacetic acid intermediates.

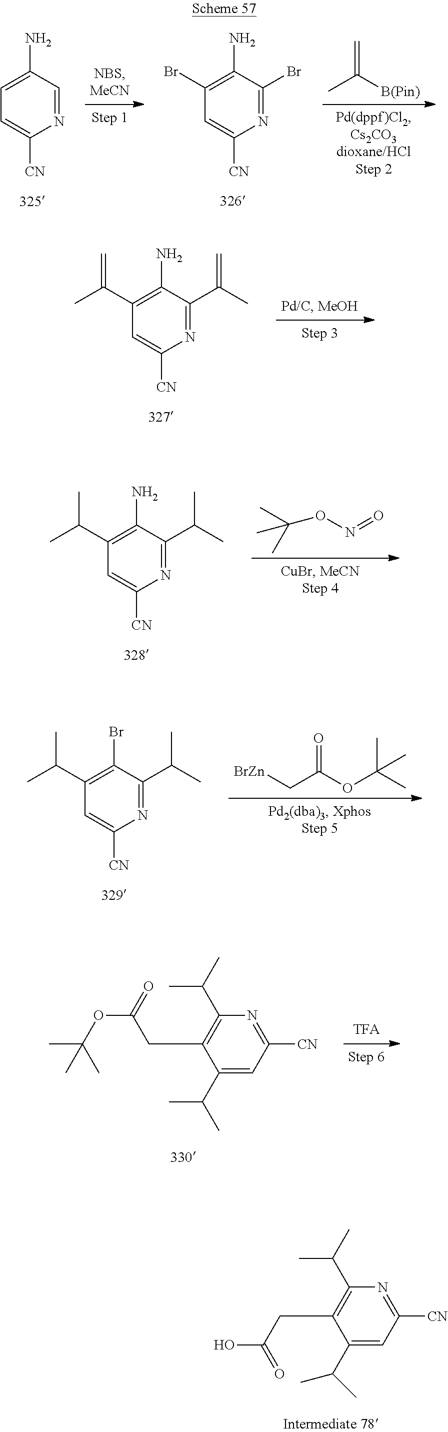

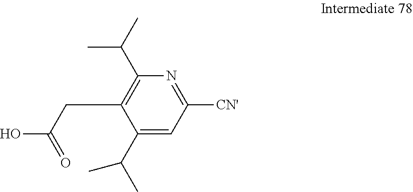

### 2-(6-Cyano-2,4-diisopropylpyridin-3-yl)acetic Acid

Steps 1-6 used similar procedures for converting compound 247′ to intermediate 66′ shown in Scheme 46 to afford intermediate 78′ from compound 325′. MS-ESI: 247 (M+1).

TABLE 24

| | | | Exact |
|---|---|---|---|
| Intermediate | | | Mass |
| # | Structure | IUPAC Name | [M − H]⁻ |
| Intermediate 79′ | 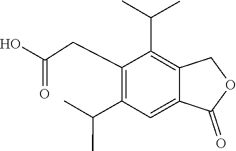 [SMILES: CC(C)c1cc2c(c(C(C)C)c1CC(=O)O)COC2=O] | 2-(4,6-Diisopropyl-1-oxo-1,3-dihydroisobenzofuran-5-yl)acetic acid | 275 |

The Intermediates in the following Table were prepared using the similar procedures for converting compound 325′ to Intermediate 78′ shown in Scheme 57 from appropriated starting materials.

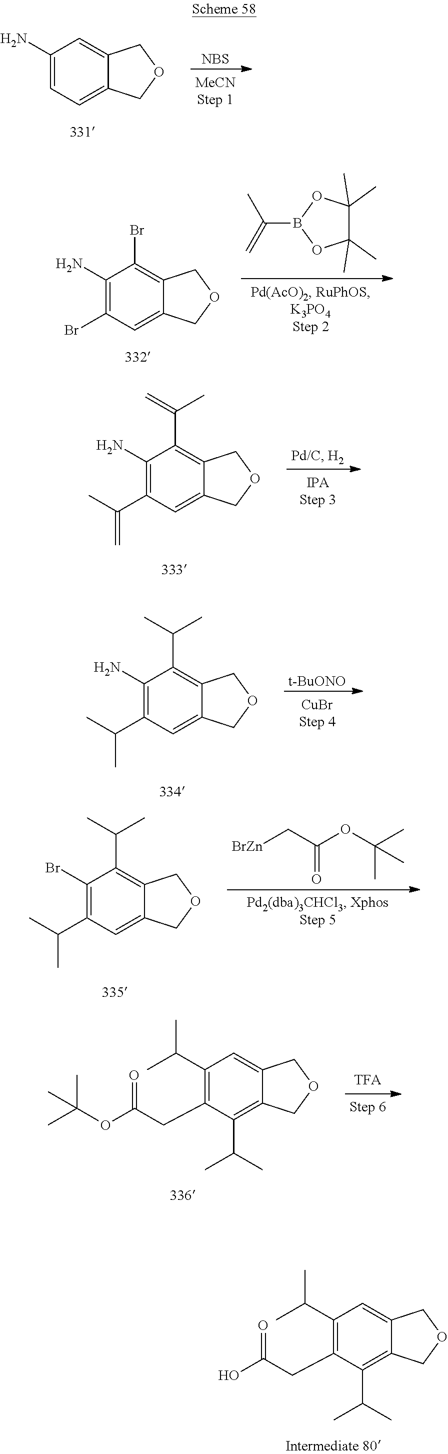

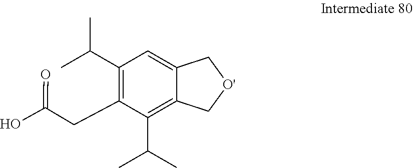

### 2-(4,6-Diisopropyl-1,3-dihydroisobenzofuran-5-yl)acetic Acid

Step 1 used similar procedures for converting compound 325′ to compound 326′ shown in Scheme 57 to afford compound 332′ from compound 331′. MS-ESI: 292/294/296 (M+1).

### Step 2: 4,6-Di(prop-1-en-2-yl)-1,3-dihydroisobenzofuran-5-amine

To a stirred solution of 4,6-dibromo-1,3-dihydroisobenzofuran-5-amine (270 g, 922 mmol) in toluene (2.7 L) and H₂O (1.35 L) in a 10-L 4-necked round-bottom flask under nitrogen was added 4,4,5,5-tetramethyl-2-(prop-1-en-2-yl)-1,3,2-dioxaborolane (464 g, 2.76 mol) and K₃PO₄ (586 g, 2.76 mol). Then Pd(AcO)₂ (10.4 g, 46.1 mmol), RuPhos (43 g, 92.2 mmol) was added rapidly. The reaction was degassed again. The resulting solution was stirred for 1 h at 90° C. The reaction mixture was cooled to RT with a water/ice bath. The solids were filtered out. The resulting solution was diluted with of EtOAc (2.7 L). The organic layer was washed with H₂O (1.5 L). The organic layer was dried over anhydrous Na₂SO₄ and concentrated under vacuum. The residue was eluted from silica gel with EtOAc/hexane (1:50). This resulted in 157 g (83%) of the title compound as light yellow oil. MS-ESI: 216 (M+1).

### Step 3: 4,6-Diisopropyl-1,3-dihydroisobenzofuran-5-amine

To a stirred solution of 4,6-di(prop-1-en-2-yl)-1,3-dihydroisobenzofuran-5-amine (147 g, 684 mmol) in IPA (2.20 L) in a 5-L round-bottom flask was added Pd/C (20% wt., 29.4 g). The flask was then connected to hydrogen and evacuated and refilled 3 times. The flask was then filled with hydrogen using a balloon. The resulting solution was stirred overnight at 30° C. under hydrogen with a balloon. The flask was evacuated and refilled with N₂ 3 times. The Pd/C was filtrated out. The resulting mixture was concentrated under vacuum. The residue was dissolved in DCM and dried over anhydrous Na₂SO₄. The solids were filtered out. The filtrate was concentrated under vacuum. This resulted in 130 g (85%) of the title compound as light yellow oil. MS-ESI: 220 (M+1).

Steps 4-6 used similar procedures for converting compound 328′ to intermediate 78′ shown in Scheme 57 to afford intermediate 80′ from compound 334′. MS-ESI: 261 (M−1).

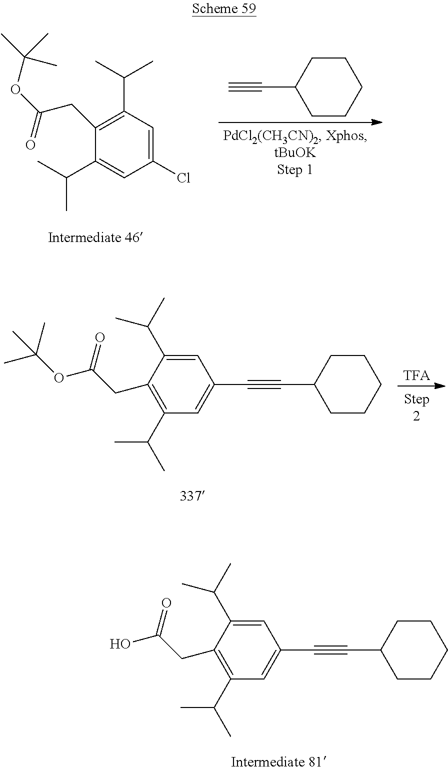

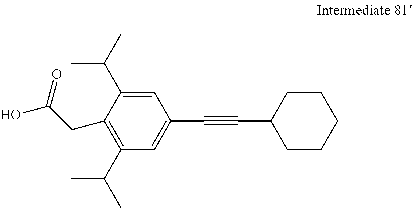 [SMILES: CC(C)c1cc(C#CC2CCCCC2)cc(C(C)C)c1CC(=O)O]

### 2-(4-(Cyclohexylethynyl)-2,6-diisopropylphenyl)acetic Acid

### Step 1: Tert-butyl 2-(4-(cyclohexylethynyl)-2,6-diisopropylphenyl)acetate

To a stirred solution of tert-butyl 2-(4-chloro-2,6-diisopropylphenyl)acetate (200 mg, 0.64 mmol) in dioxane (10 mL) in a 20-mL sealed tube purged with and maintained under nitrogen was added ethynylcyclohexane (104 mg, 0.97 mmol). Then t-BuOK (144 mg, 1.29 mmol), Xphos (61 mg, 0.13 mmol) and Pd(CH₃CN)₂Cl₂ (17 mg, 0.06 mmol) were added. The resulting solution was stirred for 2 h at 85° C. The reaction was quenched with water (10 mL). The resulting solution was extracted with 3×10 mL of DCM, the combined organic layer was dried over anhydrous Na₂SO₄ and concentrated under reduced pressure. The residue was eluted from silica gel with EtOAc/PE (1:10). This resulted in 140 mg (57%) of the title compound as brown oil. MS-ESI: 385 (M+1).

Step 2 used similar procedures for converting compound 330′ to intermediate 78′ shown in Scheme 57 to afford intermediate 81′ from compound 337′. MS-ESI: 327 (M−1).

TABLE 25

| | | | Exact |
|---|---|---|---|
| Intermediate | | | Mass |
| # | Structure | IUPAC Name | [M − H]⁻ |
| Intermediate 82′ | 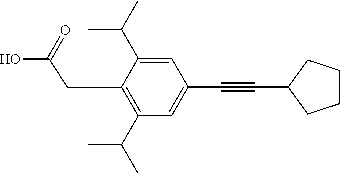 [SMILES: CC(C)c1cc(C#CC2CCCC2)cc(C(C)C)c1CC(=O)O] | 2-(4-(Cyclopentylethynyl)-2,6-diisopropylphenyl)acetic acid | 311 |
| Intermediate 83′ | 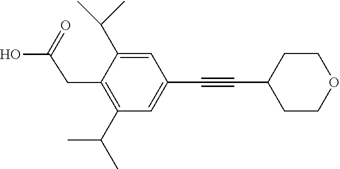 [SMILES: CC(C)c1cc(C#CC2CCOCC2)cc(C(C)C)c1CC(=O)O] | 2-(2,6-Diisopropyl-4-((tetrahydro-2H-pyran-4- yl)ethynyl)phenyl)acetic acid | 328 |

The Intermediates in the following Table were prepared using the similar procedures for converting Intermediate 46′ to Intermediate 81′ shown in Scheme 59 from appropriated starting materials.

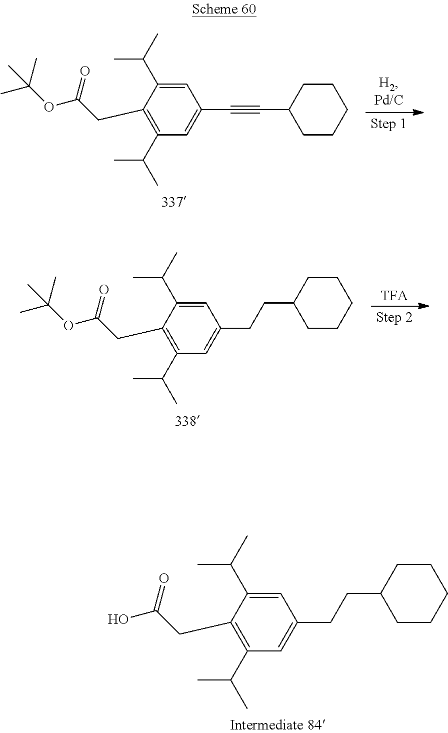 [SMILES: CC(C)c1cc(C#CC2CCCCC2)cc(C(C)C)c1CC(=O)OC(C)(C)C ; CC(C)c1cc(CCC2CCCCC2)cc(C(C)C)c1CC(=O)OC(C)(C)C ; CC(C)c1cc(CCC2CCCCC2)cc(C(C)C)c1CC(=O)O ; "TFA"]

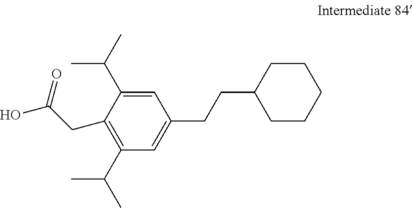 [SMILES: CC(C)c1cc(CCC2CCCCC2)cc(C(C)C)c1CC(=O)O]

### 2-(4-(2-Cyclohexylethyl)-2,6-diisopropylphenyl)acetic Acid

### Step 1: Tert-butyl 2-(4-(2-cyclohexylethyl)-2,6-diisopropylphenyl)acetate

To a stirred solution of tert-butyl 2-(4-(cyclohexylethynyl)-2,6-diisopropylphenyl)acetate (400 mg, 1.05 mmol) in MeOH (30 mL) in a 100-mL round-bottom flask, was added Pd/C (10% wt., 40 mg) at RT. The flask was evacuated and refilled with hydrogen three times with a balloon. The resulting mixture was stirred for 16 h at RT under hydrogen with a balloon. The solid was filtered out. The resulting mixture was concentrated under vacuum. The residue was eluted from silica gel with PE. This resulted in 400 mg (98.7%) of the title compound as light yellow oil. MS-ESI: 387 (M+1).

Step 2 used similar procedures for converting compound 330′ to intermediate 78′ shown in Scheme 57 to afford intermediate 84′ from compound 338′. MS-ESI: 329 (M−1).

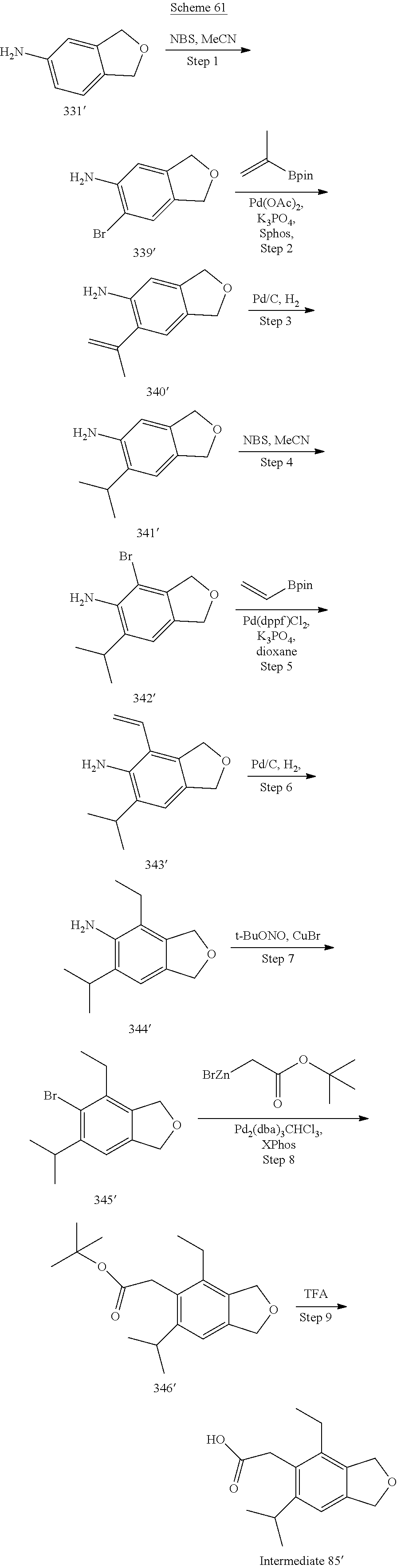

### 2-(4-Ethyl-6-isopropyl-1,3-dihydroisobenzofuran-5-yl)acetic Acid

Step 1 used similar procedures for converting compound 325′ to compound 326′ shown in Scheme 57 to afford compound 339′ from compound 331′. MS-ESI: 213/215 (M+1).

Steps 2-3 used similar procedures for converting compound 326′ to compound 328′ shown in Scheme 57 to afford compound 341′ from compound 329′. MS-ESI: 178 (M+1).

Steps 4-9 used similar procedures for converting compound 325′ to intermediate 78′ shown in Scheme 57 to afford intermediate 85′ from compound 341′. MS-ESI: 249 (M−1).

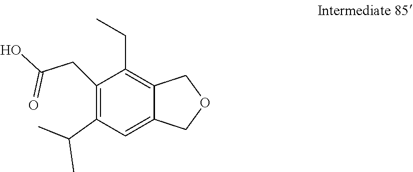 [SMILES: CCc1c2c(cc(C(C)C)c1CC(=O)O)COC2]

The Intermediates in the following Table were prepared using the similar procedures for converting compound 331′ to Intermediate 85′ shown in Scheme 61 using appropriate reagents.

TABLE 26

| Intermediate # | Structure | IUPAC Name | Exact Mass [M − H]⁻ |
|---|---|---|---|
| Intermediate 86′ | 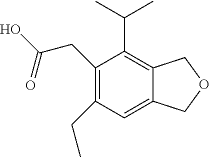 [SMILES: CCc1cc2c(c(C(C)C)c1CC(=O)O)COC2] | 2-(6-Ethyl-4-isopropyl-1,3- dihydroisobenzofuran-5-yl)acetic acid | 247 |

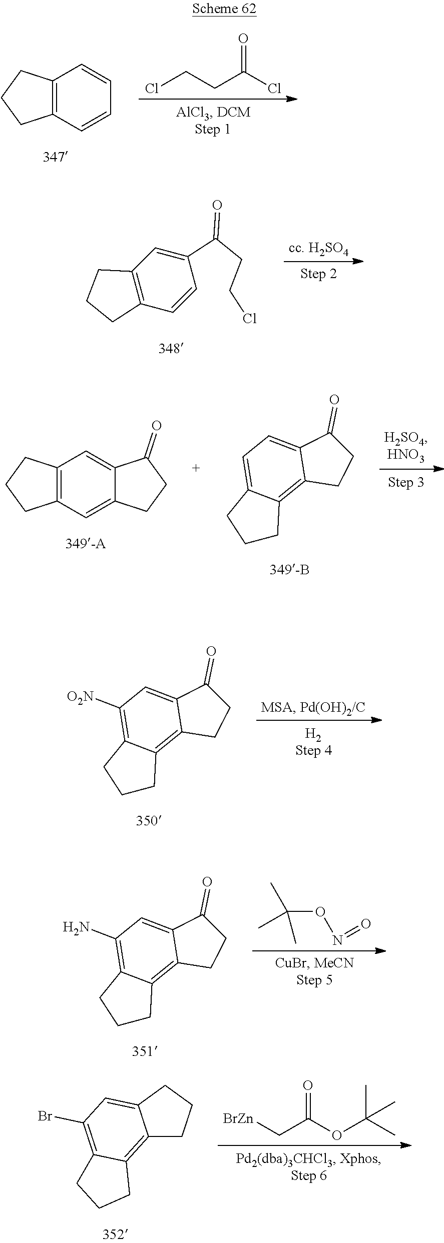

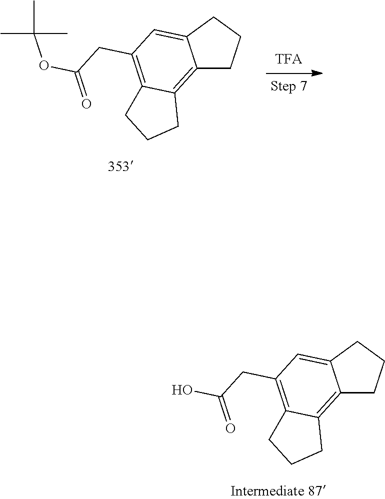 [SMILES: "TFA" ; CC(C)(C)OC(=O)Cc1cc2c(c3c1CCC3)CCC2 ; O=C(O)Cc1cc2c(c3c1CCC3)CCC2]

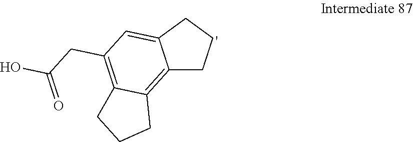 [SMILES: O=C(O)Cc1cc2c(c3c1CCC3)CCC2]

### 2-(1,2,3,6,7,8-Hexahydro-as-indacen-4-yl)acetic Acid

### Step 1: 3-Chloro-1-(2,3-dihydro-1H-inden-5-yl)propan-1-one

To a stirred solution of AlCl₃ (111 g, 834 mmol) in DCM (1.2 L) in a 3000-mL round-bottom flask was added a solution of 2,3-dihydro-1H-indene (90 g, 762 mmol) and 3-chloropropanoyl chloride (96.3 g, 759 mmol) in DCM (300 mL) dropwise at −10° C. in 30 min. The resulting solution was stirred for 16 h at RT. Then the reaction mixture was added dropwise to cold HCl (3M, 1200 mL) over 45 min at −10° C. The resulting solution was extracted with 3×600 mL of DCM and the organic layers were combined, dried over anhydrous Na₂SO₄, then concentrated under vacuum. This resulted in 161 g (crude) of the title compound as a yellow solid. The crude product was used in the next step. MS-ESI: 209 (M+1).

### Step 2: 2,3,6,7-Tetrahydro-s-indacen-1(5H)-one and 1,2,7,8-tetrahydro-as-indacen-3(6H)-one

To a stirred solution of 3-chloro-1-(2,3-dihydro-1H-inden-5-yl)propan-1-one (161 g, 759 mmol) in conc. H₂SO₄ (900 mL) in a 2000-mL round-bottom flask. The resulting solution was stirred for 16 h at 55° C. and then was quenched by adding the reaction mixture carefully to 4.5 L of water/ice. The solids were collected by filtration and dried over infrared lamp for 24 h. The mixture was eluted from silica gel with EtOAc/PE (1:200). This resulted in 112 g (85%) of 2,3,6,7-tetrahydro-s-indacen-1(5H)-one as a yellow solid and 9.8 g of 1,2,7,8-tetrahydro-as-indacen-3(6H)-one as a yellow solid. MS-ESI: 173 (M+1). Compound 349A: ¹H NMR (400 MHz, DMSO-d₆) δ 7.44 (s, 1H), 7.39 (s, 1H), 3.20-2.75 (m, 6H), 2.70-2.60 (m, 2H), 2.20-1.90 (m, 2H). Compound 349B: ¹H NMR (400 MHz, DMSO-d₆) δ 7.49 (d, J=8.0 Hz, 1H), 7.31 (d, J=8.0 Hz, 1H), 3.20-2.90 (m, 4H), 2.90-2.75 (m, 2H), 2.70-2.60 (m, 2H), 2.20-1.90 (m, 2H)

### Step 3: 5-Nitro-1,2,7,8-tetrahydro-as-indacen-3(6H)-one

To a stirred solution of 1,2,7,8-tetrahydro-as-indacen-3(6H)-one (9.8 g, 46.5 mmol) in conc. H₂SO₄ (50 mL) in a 250-mL round-bottom flask was added HNO₃ (5.85 g, 92.9 mmol) dropwise over 10 min at 0° C. The resulting solution was stirred for 1 h at 0° C. The reaction mixture was slowly added to a mixture of water/ice (100 mL) and DCM (50 mL) with ice bath cooling. The organic layer was collected, dried over Na₂SO₄ and concentrated under vacuum. This resulted in 11 g (89%) of the title compound as a yellow solid.

### Step 4: 1,2,3,6,7,8-Hexahydro-as-indacen-4-amine

To a stirred solution of 5-nitro-1,2,7,8-tetrahydro-as-indacen-3(6H)-one (2.17 g, 10 mmol) in MeOH (30 mL) in a 100-mL round-bottom flask was added MSA (1.15 g, 12 mmol). Then Pd(OH)₂/C (20% wt., 550 mg) was added. The flask was evacuated and refilled three times with hydrogen. The resulting mixture was stirred for 16 h at RT under hydrogen with a balloon. The solids were filtered out and washed with MeOH. The filtrate and wash were diluted with water (100 mL) and the pH was adjusted to 11 with 2 N NaOH solution. The resulting slurry was filtered and the filter cake was eluted from silica gel with EtOAc/PE (1:5). This resulted in 1.38 g (80%) of the title compound as a light yellow solid. MS-ESI: 174 (M+1).

Steps 5-7 used similar procedures for converting compound 328′ to intermediate 78′ shown in Scheme 57 to afford Intermediate 87′ from compound 349′-B. MS-ESI: 215 (M−1).

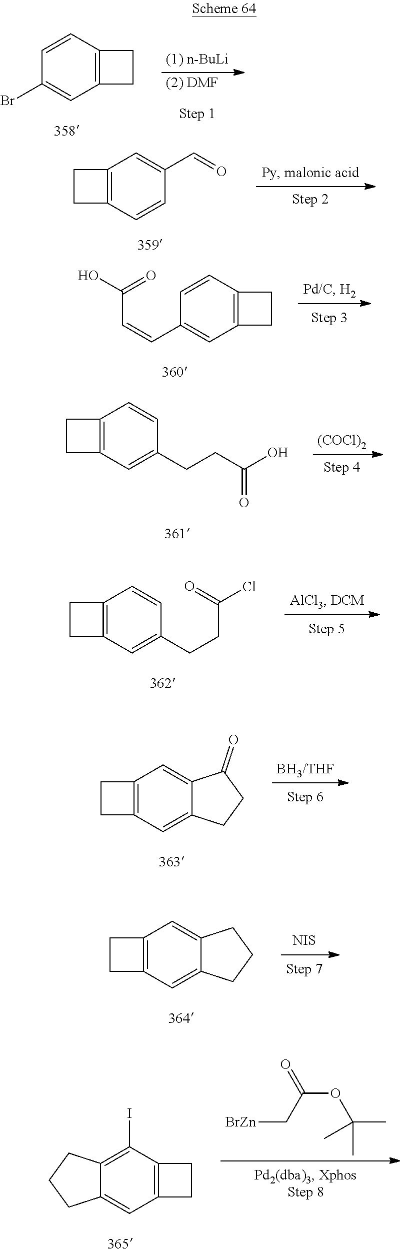

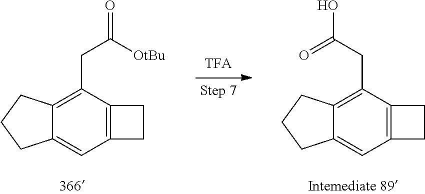 [SMILES: "TFA" ; CC(C)(C)OC(=O)Cc1c2c(cc3c1CC3)CCC2 ; O=C(O)Cc1c2c(cc3c1CC3)CCC2]

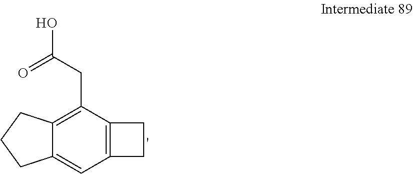 [SMILES: O=C(O)Cc1c2c(cc3c1CC3)CCC2]

### 2-(2,4,5,6-Tetrahydro-1H-cyclobuta[f]inden-3-yl)acetic Acid

### Step 1: Bicyclo[4.2.0]octa-1(6),2,4-triene-3-carbaldehyde

To a stirred solution of 3-bromobicyclo[4.2.0]octa-1(6),2,4-triene (70 g, 382 mmol) in THF (300 mL) in a 500-mL round-bottom flask under nitrogen was added n-BuLi in hexane (2.5 M, 184 mL, 459 mmol) dropwise at −70° C. After addition, the reaction mixture was stirred at this temperature for 30 min. To this solution was added DMF (36.3 g, 497 mmol) dropwise with stirring at −70° C. The resulting solution was stirred for 30 min at −70° C. in a liquid nitrogen/EtOH bath. The reaction was slowly warmed to RT and then quenched by the addition of 100 mL of water. The resulting solution was extracted with 3×200 ml of DCM. The organic layers were combined and dried over anhydrous Na₂SO₄, and concentrated under reduced pressure. This resulted in 50 g (98.9%) of the title compound as light yellow oil. MS-ESI: 133 (M+1).

### Step 2: (Z)-3-(bicyclo[4.2.0]octa-1(6),2,4-trien-3-yl)acrylic Acid

To a stirred solution of bicyclo[4.2.0]octa-1(6),2,4-triene-3-carbaldehyde (1.7 g, 12.9 mmol) in pyridine (20 mL) in a 250-mL round-bottom flask under nitrogen was added malonic acid (1.99 g, 19.2 mmol) and piperidine (110 mg, 1.29 mmol). The resulting solution was stirred overnight at 90° C. in an oil bath. The resulting mixture was concentrated under vacuum. This resulted in 2.1 g (93.7%) of the title compound as a light yellow solid. MS-ESI: 173 (M−1).

### Step 3: 3-(Bicyclo[4.2.0]octa-1(6),2,4-trien-3-yl)propanoic Acid

To a stirred solution of 2-(Z)-3-[bicyclo[4.2.0]octa-1(6),2,4-trien-3-yl]prop-2-enoic acid (2.1 g, 12.1 mmol) in MeOH (50 mL) in a 250-mL round-bottom flask was added Pd/C (10% wt., 200 mg). The flask was evacuated and flushed three times with hydrogen. The resulting solution was stirred for 12 h at RT. The Pd/C catalysts were filtered out, the filtrate was concentrated under vacuum. This resulted in 2.1 g (98.9%) of the title compound as a yellow solid. MS-ESI: 175 (M−1).

### Step 4: 3-(Bicyclo[4.2.0]octa-1(6),2,4-trien-3-yl)propanoyl Chloride

To a stirred solution of 3-[bicyclo[4.2.0]octa-1(6),2,4-trien-3-yl]propanoic acid (10 g, 56.8 mmol) in DCM (100 mL) in a 250-mL round-bottom flask under nitrogen was added oxalyl chloride (7.2 g, 56.8 mmol) dropwise at 0° C. The resulting solution was stirred for 2 h at 0° C. in a water/ice bath. The resulting mixture was use in next step without further purification.

### Step 5: 1,2,5,6-Tetrahydro-4H-cyclobuta[f]inden-4-one

To a stirred solution of 3-[bicyclo[4.2.0]octa-1(6),2,4-trien-3-yl]propanoyl chloride in DCM (100 mL, from step 4) in a 500-mL round-bottom flask was added AlCl₃ (7.5 g, 56.8 mmol) in portions at 0° C. over 10 min. The resulting solution was stirred for 2 h at 0° C. in a water/ice bath. The reaction was then quenched by the addition of 200 mL of water. The resulting solution was extracted with 2×200 mL of DCM. The organic layers were combined, dried over anhydrous Na₂SO₄, and concentrated under vacuum. The residue was eluted from silica gel with EtOAc/PE (1:20 to 1:15). This resulted in 7.7 g (86.1%) of the title compound as a yellow solid. ¹H NMR (300 MHz, CDCl₃) δ 7.45 (s, 1H), 7.17 (s, 1H), 3.23-3.21 (m, 4H), 3.18-3.00 (m, 2H), 2.73-2.63 (m, 2H).

### Step 6: 2,4,5,6-Tetrahydro-1H-cyclobuta[f]indene

To a stirred solution of 1,2,5,6-tetrahydrocyclobuta[f]inden-4-one (20 g, 126 mmol) in THF (200 mL) in a 500-mL round-bottom flask under nitrogen was added BH₃-Me₂S (10 M) (25.3 mL, 253 mmol) dropwise at 0° C. in an ice bath. The resulting solution was stirred for 14 h at 70° C. in an oil bath. The reaction was then quenched by the addition of 20 mL of MeOH. The resulting mixture was concentrated. The residue was eluted from silica gel with EtOAc/PE (1:100 to 1:50). This resulted in 15 g (82.3%) of the title compound as a light yellow solid. ¹H NMR (300 MHz, CDCl₃) δ 6.95 (s, 2H), 3.10 (s, 4H), 2.88 (t, J=7.4 Hz, 4H), 2.09-1.99 (m, 2H).

### Step 7: 3-Iodo-2,4,5,6-tetrahydro-1H-cyclobuta[f]indene

To a stirred solution of 2,4,5,6-tetrahydro-1H-cyclobuta[f]indene (15 g, 104 mmol) in DCE (200 mL) in a 500-mL round-bottom flask under nitrogen was added NIS (46.8 g, 208 mmol). This was followed by the addition of AcOH (60 mL) and water (0.5 mL) The resulting solution was stirred for 14 h at 50° C. in an oil bath. The reaction was quenched with 30% Na₂SO₃ (aq.) (100 mL). The mixture was extracted with 3×100 mL of DCM. The organic layers were combined and dried over anhydrous Na₂SO₄, then concentrated. The residue was eluted from silica gel with PE. This resulted in 8.2 g (29.2%) of the title compound as yellow solid. MS-ESI: 271 (M+1).

Steps 8-9 used similar procedures for converting compound 329′ to intermediate 78′ shown in Scheme 57 to afford intermediate 89′ from compound 365′. MS-ESI: 201 (M−1).

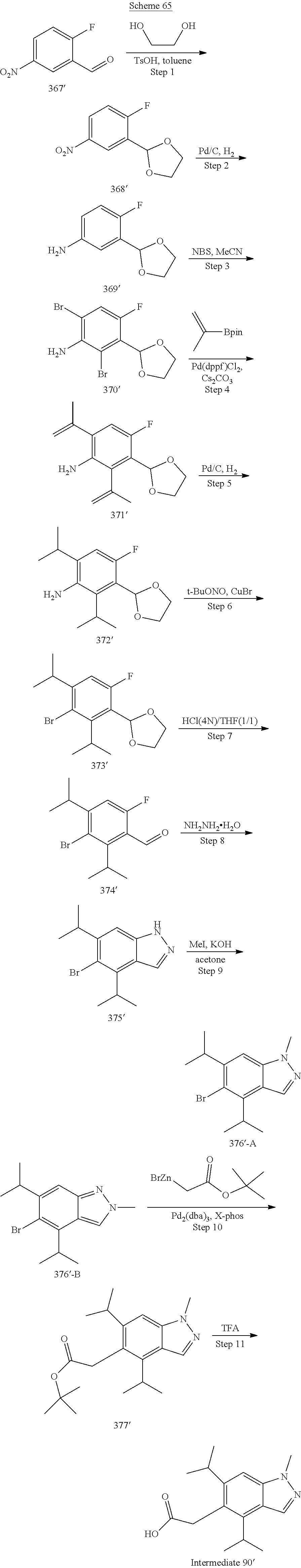

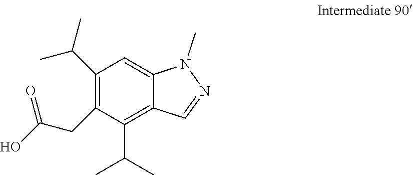 [SMILES: CC(C)c1cc2c(cnn2C)c(C(C)C)c1CC(=O)O]

### 2-(4,6-Diisopropyl-1-methyl-1H-indazol-5-yl)acetic Acid

### Step 1: 2-(2-Fluoro-5-nitrophenyl)-1,3-dioxolane

To a stirred solution of 2-fluoro-5-nitrobenzaldehyde (7.0 g, 41.4 mmol4) in toluene (150 mL), TsOH (7.13 g, 41.4 mmol) in a 250-mL round-bottom flask under nitrogen was added ethane-1,2-diol (12.9 g, 207 mmol). The resulting solution was stirred overnight at 110° C. in an oil bath. The resulting solution was diluted with 100 mL of H₂O. The resulting solution was extracted with 3×200 mL of EtOAc and the organic layers were combined and concentrated under vacuum. The residue was eluted from a silica gel column with DCM/MeOH (100:1). This resulted in 8.2 g (93%) of the title compound as an off-white solid. MS-ESI: 214 (M+1).

### Step 2: 3-(1,3-Dioxolan-2-yl)-4-fluoroaniline

To a stirred solution of 2-(2-fluoro-5-nitrophenyl)-1,3-dioxolane (6.39 g, 30 mmol) in MeOH (150 mL) in a 500-mL round-bottom flask under nitrogen was added Pd/C (10% wt., 650 mg). The flask was evacuated and refilled 3 times with hydrogen using a balloon. The resulting solution was stirred overnight at RT under hydrogen with a balloon. The flask was evacuated and refilled with nitrogen 3 times. The Pd/C was filtrated out and the filter cake was washed with DCM (3×50 mL). The filtrate and wash were combined and concentrated under reduced pressure. The crude product was eluted from silica gel from EtOAc/PE (1:1). This resulted in 4.86 g (88.6%) of the title compound as a yellow solid. MS-ESI: 220 (M+1).

Steps 3-6 used similar procedures for converting compound 325′ to compound 329′ shown in Scheme 57 to afford compound 373′ from compound 369′. MS-ESI: 311/313 (M+1).

### Step 7: 3-Bromo-6-fluoro-2,4-diisopropylbenzaldehyde

To a stirred solution of 2-(3-bromo-6-fluoro-2,4-diisopropylphenyl)-1,3-dioxolane (1.0 g, 3.02 mmol) in THF (10 mL) in a 50-mL round-bottom flask was added HCl (4M, 10 mL). The resulting solution was stirred for 2 h at RT. The resulting solution was diluted with 10 mL of H₂O. The resulting solution was extracted with 3×30 mL of EtOAc, the combined organic layer was dried over Na₂SO₄ and concentrated under reduced pressure. The residue was eluted from silica gel with PE. This resulted in 800 mg (92.3%) of the title compound as light yellow oil. MS-ESI: 287/289 (M+1).

### Step 8: 5-Bromo-4,6-diisopropyl-1H-indazole

To a stirred solution of 3-bromo-6-fluoro-2,4-diisopropylbenzaldehyde (760 mg, 2.65 mmol) in DMSO (5.0 mL) in a 50-mL round-bottom flask under nitrogen was added hydrazine hydrate (80% wt., 3.0 mL). The resulting solution was stirred for overnight at 120° C. in an oil bath. The resulting solution was diluted with 100 mL of EtOAc and 50 mL of H₂O. the organic layer combined and washed with 30 mL of water, then dried over Na₂SO₄ and concentrated under reduced pressure. The residue was eluted from silica gel with EtOAc/PE (1:4). This resulted in 440 mg (59%) of the title compound as a light yellow solid. MS-ESI: 281/283 (M+1).

### Step 9: 5-Bromo-4,6-diisopropyl-1-methyl-1H-indazole

To a stirred solution of 5-bromo-4,6-diisopropyl-1H-indazole (520 mg, 1.85 mmol) in acetone (10 mL) in a 50-mL round-bottom flask was added KOH (311 mg, 5.55 mmol) in several batches at 0° C. The resulting solution was stirred for 30 min at 0° C. in a water/ice bath. To this was added CH₃I (525 mg, 3.7 mmol) dropwise with stirring at 0° C. The resulting solution was allowed to react, with stirring, for an additional 2 h at RT. The resulting solution was diluted with 20 mL of H₂O. The resulting solution was extracted with 3×20 mL of EtOAc and the organic layers were combined and concentrated under vacuum. The residue was eluted from silica gel with EtOAc/PE (1:4). This resulted in 240 mg (43.9%) of 5-bromo-4,6-diisopropyl-1-methyl-1H-indazole as a light yellow solid. This resulted in 180 mg (32.9%) of 5-bromo-4,6-diisopropyl-2-methyl-2H-indazole as light yellow oil. The isomers were assigned based on ¹H-¹H NOESY. Compound 376A: ¹H NMR (400 MHz, DMSO-d₆) δ 8.26 (s, 1H), 7.48 (s, 1H), 4.05 (s, 3H), 3.90-3.75 (m, 1H), 3.60-3.40 (m, 1H), 1.60-1.35 (m, 6H), 1.35-1.20 (m, 6H). Compound 376B: ¹H NMR (400 MHz, DMSO-d₆) δ 8.62 (s, 1H), 7.38 (s, 1H), 4.15 (s, 3H), 3.90-3.75 (m, 1H), 3.50-3.30 (m, 1H), 1.45-1.30 (m, 6H), 1.29-1.10 (m, 6H)

Steps 10-11 used similar procedures for converting compound 329′ to intermediate 78′ shown in Scheme 57 to afford intermediate 90′ from compound 376′-A. MS-ESI: 273 (M−1).

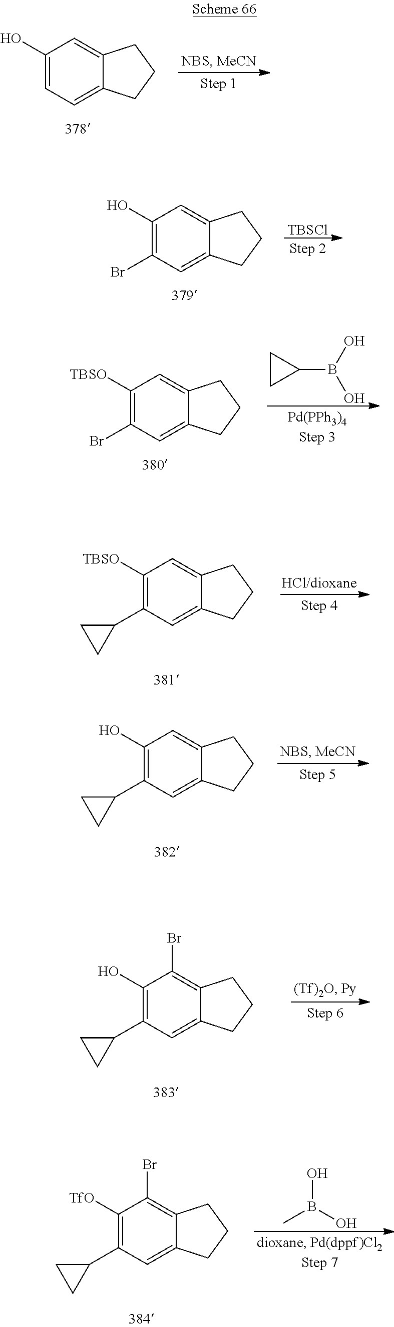

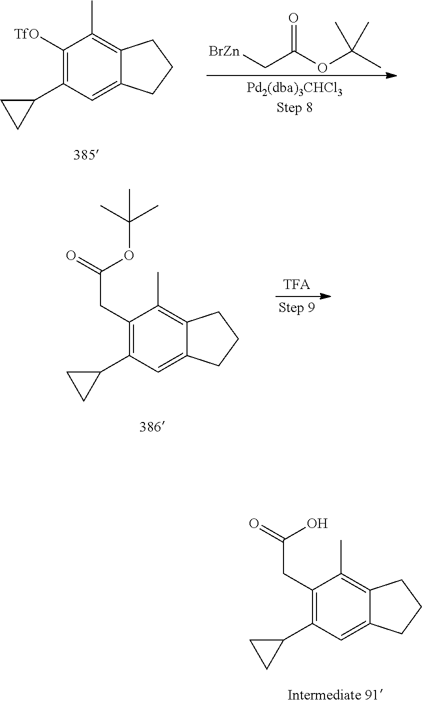

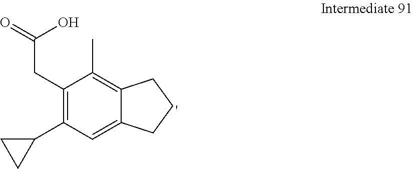 [SMILES: Cc1c2c(cc(C3CC3)c1CC(=O)O)CCC2]

### 2-(6-Cyclopropyl-4-methyl-2,3-dihydro-1H-inden-5-yl)acetic Acid

Step 1 used similar procedures for converting compound 325′ to compound 326′ shown in Scheme 57 to afford compound 379′ from compound 378′. MS-ESI: 211/213 (M+1).

### Step 2: ((6-Bromo-2,3-dihydro-1H-inden-5-yl)oxy)(tert-butyl)dimethylsilane

To a stirred solution of 2,3-dihydro-1H-inden-5-ol (8.0 g, 37.5 mmol) in DMF (50 mL) in a 250-mL 3-necked round-bottom flask under nitrogen was added TBSCl (6.79 g, 45 mmol) and imidazole (5.11 g, 75.1 mmol). The resulting solution was stirred for 2 h at RT. The resulting solution was diluted with 200 mL of water and extracted with 2×100 mL of DCM. The combined extract was washed with 3×100 ml of water and concentrated under vacuum. The residue was eluted from silica gel with EtOAc/PE (1:100). This resulted in 10 g (81.4%) of the title compound as colorless liquid. MS-ESI: 327/329 (M+1).

### Step 3: Tert-butyl((6-cyclopropyl-2,3-dihydro-1H-inden-5-yl)oxy)dimethylsilane

To a stirred solution of ((6-bromo-2,3-dihydro-1H-inden-5-yl)oxy)(tert-butyl)dimethylsilane (5.0 g, 15.3 mmol) in toluene (50 mL) and H₂O (10 mL) in a 250-mL 3-necked round-bottom flask under nitrogen was added cyclopropylboronic acid (2.62 g, 0.031 mmol). Then Pd(PPh₃)₄ (884 mg, 0.77 mmol) and Na₂CO₃ (3.24 g, 31 mmol) were added at RT. The resulting solution was stirred for 14 h at 90° C. under nitrogen. The resulting solution was diluted with 100 mL of water and extracted with 2×200 mL of DCM. The organic layers were combined, dried with anhydrous Na₂SO₄ and concentrated under vacuum. The residue was eluted from silica gel with EtOAc/PE (1:100). This resulted in 4.0 g (90.8%) of the title compound as colorless liquid. MS-ESI: 289 (M+1).

### Step 4: 6-Cyclopropyl-2,3-dihydro-1H-inden-5-ol

To a stirred solution of tert-butyl((6-cyclopropyl-2,3-dihydro-1H-inden-5-yl)oxy)dimethylsilane (4.5 g, 15.6 mmol) in dioxane (10 mL) in a 100-mL round-bottom flask under nitrogen was added HCl in dioxane (4M, 5.0 mL). The resulting solution was stirred for 3 h at RT. The resulting solution was diluted with 60 mL of water and extracted with 3×100 mL of DCM. The organic layers were combined, dried with Na₂SO₄ and concentrated under vacuum. The residue was eluted from silica gel with EtOAc/PE (1:100 to 10:90). This resulted in 2.3 g (85%) of the title compound as a white solid. MS-ESI: 173 (M−1).

### Step 5: 4-Bromo-6-cyclopropyl-2,3-dihydro-1H-inden-5-ol

To a stirred solution of 6-cyclopropyl-2,3-dihydro-1H-inden-5-ol (2.1 g, 12.1 mmol) in MeCN (30 mL) in a 100-mL round-bottom flask under nitrogen was added NBS (2.15 g, 12.1 mmol) in portions at RT. The resulting solution was stirred for 2 h at 0° C. in a water/ice bath. The resulting solution was diluted with 50 mL of water and extracted with 3×100 mL of DCM. The organic layers were combined, dried with Na₂SO₄ and concentrated under vacuum. The residue was eluted from silica gel with EtOAc/PE (1:100). This resulted in 1.5 g (49%) of the title compound as a yellow solid. MS-ESI: 251/253 (M−1).

### Step 6: 4-Bromo-6-cyclopropyl-2,3-dihydro-1H-inden-5-yl Trifluoromethanesulfonate

To a stirred solution of 4-bromo-6-cyclopropyl-2,3-dihydro-1H-inden-5-ol (1.5 g, 5.93 mmol) in DCM (20 mL) in a 100-mL round-bottom flask under nitrogen was added TEA (2.40 g, 23.7 mmol). This was followed by the addition of triflic anhydride (3.34 g, 12 mmol) dropwise with stirring at 0° C. The resulting solution was stirred for 2 h at 0° C. in a water/ice bath. The reaction was then quenched by the addition of 50 mL of water/ice. The resulting solution was extracted with 3×100 mL of DCM and the organic layers were combined and concentrated under vacuum. The residue was eluted from silica gel with EtOAc/PE (1:80). This resulted in 2.0 g (88%) of the title compound as brown oil. MS-ESI: 385/387 (M+1).

### Step 7: 6-cyclopropyl-4-methyl-2,3-dihydro-1H-inden-5-yl Trifluoromethanesulfonate

To a stirred solution of 4-bromo-6-cyclopropyl-2,3-dihydro-1H-inden-5-yl trifluoromethanesulfonate (200 mg, 0.52 mmol) in dioxane (10 mL) and H₂O (1 mL) in a 50-mL round-bottom flask under nitrogen was added methylboronic acid (147 mg, 2.45 mmol). Then Cs₂CO₃ (508 mg, 1.56 mmol) and Pd(dppf)Cl₂ (38 mg, 0.052 mmol) were added. The resulting solution was stirred for 14 h at 100° C. in an oil bath. The resulting solution was diluted with 30 mL of water and extracted with 3×100 mL of DCM. The organic layers were combined and concentrated under vacuum. The residue was eluted from silica gel with EtOAc/hexane (1:80 to 1:50). This resulted in 100 mg (76.7%) of the title compound as light yellow oil. MS-ESI: 321 (M+1).

Step 8-9 used similar procedures for converting compound 329′ to intermediate 78′ shown in Scheme 57 to afford intermediate 91′ from compound 385′. MS-ESI: 229 (M−1).

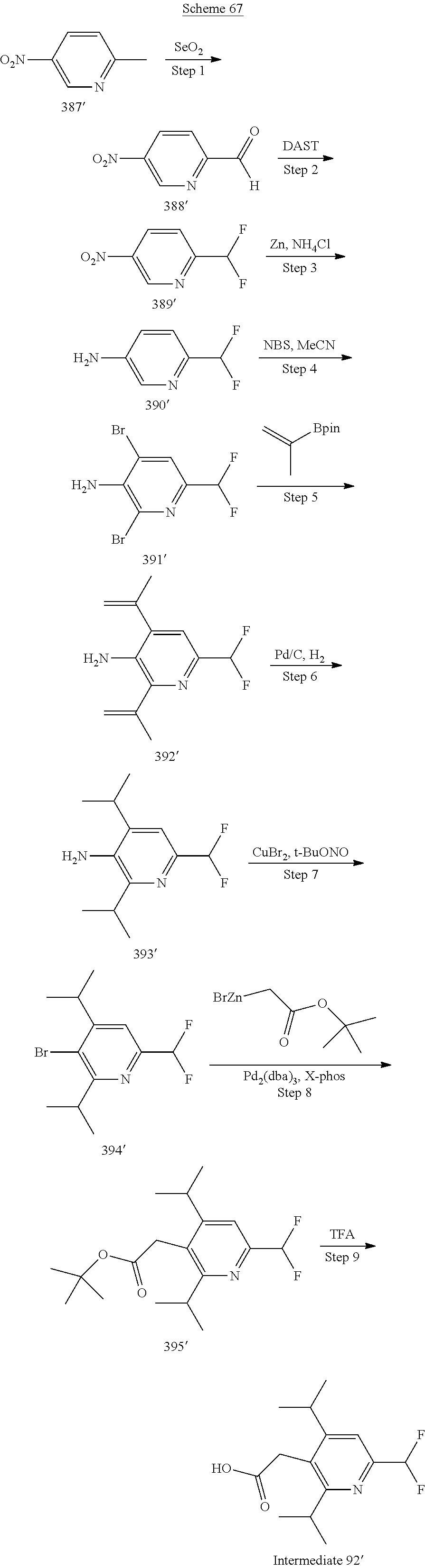

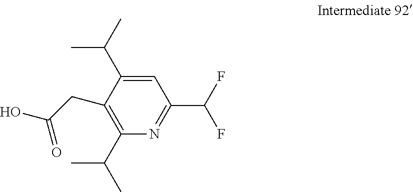 [SMILES: CC(C)c1cc(C(F)F)nc(C(C)C)c1CC(=O)O]

### 2-(6-(Difluoromethyl)-2,4-diisopropylpyridin-3-yl)acetic Acid

### Step 1: 5-Nitropicolinaldehyde

To a stirred solution of 2-methyl-5-nitropyridine (30 g, 217 mmol) in DCM (500 mL) in a 1-L round-bottom flask under nitrogen was added SeO₂ (48.2 g, 434 mmol). The resulting solution was stirred for 16 h at RT. The solids were filtered out. The resulting mixture was concentrated under vacuum. The residue was eluted from silica gel with EtOAc/PE (1:1). This resulted in 15 g (46%) of the title compound as a yellow solid. MS-ESI: 153 (M+1).

### Step 2: 2-(Difluoromethyl)-5-nitropyridine

To a stirred solution of 5-nitropicolinaldehyde (15 g, 98 mmol) in DCM (1.0 L) in a 2-L round-bottom flask under nitrogen was added DAST (12.7 g, 78 mmol) dropwise at 0° C. The resulting solution was stirred for 16 h at RT. The reaction was poured into 300 mL of water/ice. The resulting solution was extracted with 3×300 mL of DCM. The combined extract was combine and dried over anhydrous Na₂SO₄. The solids were filtered out. The resulting mixture was concentrated under vacuum. The residue was eluted from silica gel with EtOAc/PE (1:1). This resulted in 15 g (87%) of the title compound as a yellow solid. MS-ESI: 175 (M+1).

### Step 3: 6-(Difluoromethyl)pyridin-3-amine

To a stirred solution of 2-(difluoromethyl)-5-nitropyridine (15 g, 86 mmol) in THF (500 mL) in a 1-L 3-necked round-bottom flask under nitrogen was added NH₄Cl (9.22 g, 172 mmol) and Zn powder (11.3 g, 172 mmol). The resulting solution was stirred for 16 h at RT. The solids were filtered out. The resulting mixture was concentrated under vacuum. The residue was eluted from silica gel with EtOAc/PE (1:1). This resulted in 7.0 g (56%) of the title compound as a yellow solid. MS-ESI: 145 (M+1).

Steps 4-9 used similar procedures for converting compound 325′ to intermediate 78′ shown in Scheme 57 to afford intermediate 92′ from compound 390′. MS-ESI: 270 (M−1).

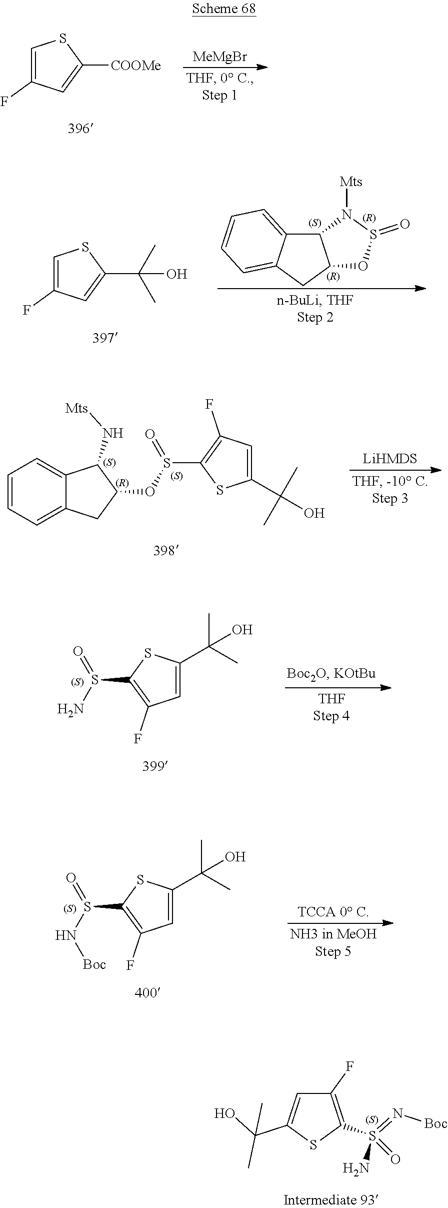

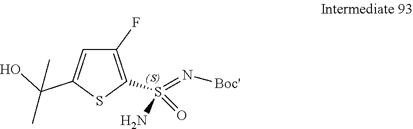

### Tert-butyl (S)-(amino(3-fluoro-5-(2-hydroxypropan-2-yl)thiophen-2-yl)(oxo)-λ⁶-sulfanylidene)carbamate

### Step 1: 2-(4-Fluorothiophen-2-yl)propan-2-ol

To a stirred solution of methyl 4-fluorothiophene-2-carboxylate (5.5 g, 34.3 mmol) in THF (55 mL) in a 250 mL 3-neck flask under nitrogen was added MeMgBr (1M in THF, 86 mL, 86 mmol) dropwise at 0° C. The resulting solution was kept stirring for 3 h at 0° C. under nitrogen. LC showed reaction was completed. The resulting mixture was quenched with cold sat. aq. NH₄Cl (100 mL) and extracted with EtOAc (3×50 mL). The combined organic layers were washed with brine (2×50 mL). The organic layer was dried over anhydrous Na₂SO₄. After filtration, the filtrate was concentrated under reduced pressure. The crude product was re-crystallized from PE/EtOAc (100:1) to afford (4.0 g, 72%) of the title compound as a light yellow solid. GCMS: 160 [M]. ¹H NMR (400 MHz, DMSO-d₆) δ 6.91 (d, J=1.7 Hz, 1H), 6.84 (d, J=1.7 Hz, 1H), 5.52 (s, 1H), 1.47 (s, 6H).

### Step 2: (1S,2R)-1-((2,4,6-trimethylphenyl)sulfonamido)-2,3-dihydro-1H-inden-2-yl (S)-3-fluoro-5-(2-hydroxypropan-2-yl)thiophene-2-sulfinate

To a stirred solution of 2-(4-fluorothiophen-2-yl)propan-2-ol (2.0 g, 12.5 mmol) in THF (30 mL) in a 100 mL 3-neck flask under nitrogen was added LDA (2M in hexane, 12.5 mL, 25 mmol) dropwise at −78° C. over 1 min. The resulting solution was kept stirring for 30 min at −78° C., this solution was assigned as A. The solution of (2R,3aS,8aR)-3-(mesitylsulfonyl)-3,3a,8,8a-tetrahydroindeno[1,2-d][1,2,3] oxathiazole 2-oxide (4.7 g, 12.5 mmol) in THF (10 mL) was transferred into the solution A, while the internal temperature of the resulting solution was kept below −65° C. under nitrogen. The resulting solution was kept stirring for 30 min at −70° C. under nitrogen. LC showed reaction to be complete. The resulting mixture was quenched with cool sat. aq. NH₄Cl (50 mL) and extracted with EtOAc (3×50 mL). The combined organic layers were washed with brine (2×50 mL). The organic layer was dried over anhydrous Na₂SO₄. After filtration, the filtrate was concentrated under reduced pressure. The crude product was re-crystallized from petroleum/EtOAc (10:1) to afford (2.6 g, 38%, de=100%) of the title compound as a white solid. MS-ESI: 560 [M+Na]⁺. ¹H NMR (400 MHz, DMSO-d₆) δ 8.19 (d, J=9.7 Hz, 1H), 7.27-7.17 (m, 2H), 7.18-7.12 (m, 1H), 7.04 (s, 2H), 7.01 (s, 1H), 6.92 (d, J=7.4 Hz, 1H), 5.86 (s, 1H), 4.98-4.89 (m, 1H), 4.80 (dd, J=9.6, 5.1 Hz, 1H), 3.22 (dd, J=16.7, 4.8 Hz, 1H), 3.08 (dd, J=16.7, 2.2 Hz, 1H), 2.61 (s, 6H), 2.28 (s, 3H), 1.49 (s, 3H), 1.48 (s, 3H).

### Step 3: (S)-3-fluoro-5-(2-hydroxypropan-2-yl)thiophene-2-sulfinamide

To a stirred solution of (1S,2R)-1-((2,4,6-trimethylphenyl)sulfonamido)-2,3-dihydro-1H-inden-2-yl-(S)-3-fluoro-5-(2-hydroxypropan-2-yl)thiophene-2-sulfinate (1.5 g, 2.79 mmol) in THF (9 mL) in 3-neck round-bottom flask under nitrogen was added a solution of NaHMDS (2M in THF, 5.6 ml, 11.2 mmol) at −10° C. under. The resulting solution was kept stirring for 1 h at −10° C. under nitrogen. LC showed reaction to be complete. The resulting mixture was quenched with AcOH (704 mg, 11.7 mmol) and MeOH (1.5 mL) below 0° C. The resulting mixture was diluted with water (50 mL) and extracted with EtOAc (3×50 mL). The combined organic layers were washed with brine (2×50 mL). The organic layer was dried over anhydrous Na₂SO₄. After filtration, the filtrate was concentrated under reduced pressure. The crude product was re-crystallized from petroleum/EtOAc (3:1) to afford (452 mg, 72%, ee=85.4%) of the title compound as a white solid. MS-ESI: 224 (M+1). ¹H NMR (400 MHz, DMSO-d₆) δ 6.92 (s, 1H), 6.61 (s, 2H), 5.70 (s, 1H), 1.46 (s, 3H), 1.45 (s, 3H).

### Step 4: Tert-butyl (S)-((3-fluoro-5-(2-hydroxypropan-2-yl)thiophen-2-yl)sulfinyl)carbamate

To a stirred solution of (S)-3-fluoro-5-(2-hydroxypropan-2-yl)thiophene-2-sulfinamide (200 mg, 0.62 mmol, ee=85.4%) in 3.0 mL of dry THF a 25 mL 3-neck round-bottom flask under nitrogen was added t-BuOK (120 mg, 0.74 mmol) at 0° C. The solution was stirred for 30 min; then the Boc₂O (195 mg, 0.62 mmol) in 1.0 mL of THF was added. The ice bath was removed and the reaction was stirred at RT for 1 h. LC showed reaction to be complete. The resulting mixture was quenched with aq. NH₄Cl (25 mL) and extracted with EtOAc (3×5 mL). The combined organic layers were washed with brine (2×5 mL). The organic layer was dried over anhydrous Na₂SO₄. After filtration, the filtrate was concentrated under reduced pressure. The crude product was re-crystallized from petroleum/EtOAc (5:1) to afford (49 mg, 17%, ee=97.4%) of the title compound as a white solid. MS-ESI: 386 [M+Na+MeCN]⁺, ¹H NMR (300 MHz, DMSO-d₆) δ 10.96 (s, 1H), 7.03 (s, 1H), 5.85 (s, 1H), 1.50 (s, 3H), 1.48 (s, 3H), 1.46 (s, 9H).

### Step 5: Tert-butyl (S)-(amino(3-fluoro-5-(2-hydroxypropan-2-yl)thiophen-2-yl)(oxo)-λ⁶-sulfaneylidene) carbamate

To a stirred solution of tert-butyl(S)-((3-fluoro-5-(2-hydroxypropan-2-yl)thiophen-2-yl) sulfinyl)carbamate (15 mg, 0.046 mmol, ee=97.4%) in dry THF (0.3 mL) in 10-mL sealed tube purged with and maintained under nitrogen. The reaction mixture was cooled to 0° C. and TCCA (3.78 mg, 0.016 mmol) was added portion-wise to avoid any exotherm and extensive over-chlorination. The reaction was stirred at 0° C. for 1 h. This solution was assigned as A. The solution A was cooled to −50° C. and the solution of NH₃ (7M in MeOH) was added dropwise via syringe. The reaction was stirred at −50° C. for 1 h. LC showed reaction to be complete. After filtration, the filtrate was purified by Prep-HPLC to afford (3.0 mg, 19%, ee=97.9%) the title compound as a white solid. MS-ESI. 339 (M+1). ¹H NMR (400 MHz, MeOH-d₄) δ 6.81 (s, 1H), 1.58 (s, 6H), 1.39 (s, 9H).

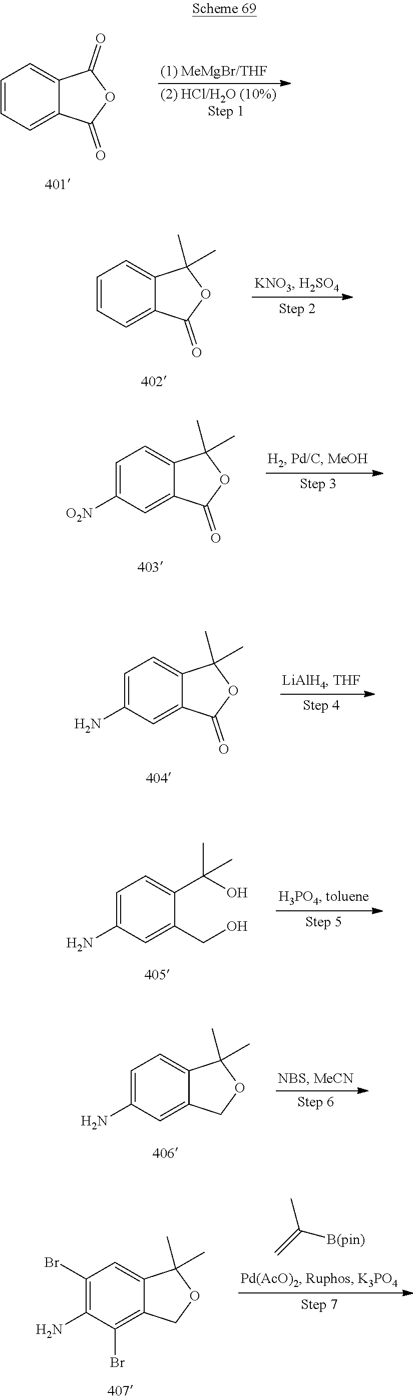

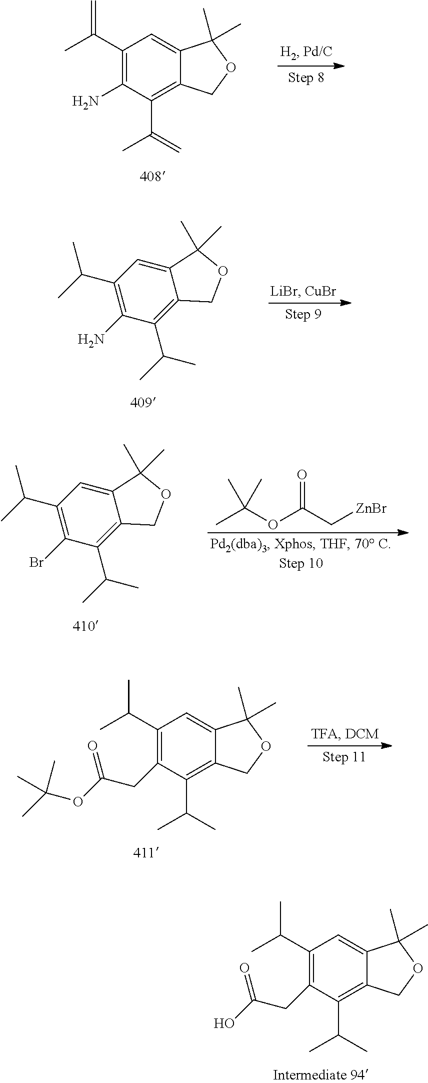

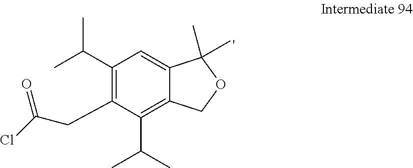 [SMILES: CC(C)c1cc2c(c(C(C)C)c1CC(=O)Cl)COC2(C)C]

### 2-(4,6-Diisopropyl-1,1-dimethyl-1,3-dihydroisobenzofuran-5-yl)acetyl Chloride

### Step 1: 3,3-Dimethylisobenzofuran-1(3H)-one

To a stirred solution of isobenzofuran-1,3-dione (20 g, 135 mmol) in THF (250 mL) in a 1 L 3-necked round-bottom flask under nitrogen was added MeMgBr in THF (3 M, 99 mL, 297 mmol) dropwise at 0° C. in an ice bath. The resulting solution was stirred for 4 h at RT. The reaction was quenched by the addition of 150 mL of HCl (10% wt.). The resulting solution was extracted with 2×100 mL of EtOAc, the combined organic phase was dried over anhydrous sodium sulfate. The solids were filtered out. The resulting mixture was concentrated under vacuum. The residue was eluted from silica gel with EtOAc/PE (1:9). This resulted in 18 g (82%) of the title compound as a yellow solid. MS-ESI: 162 (M+1).

### Step 2: 3,3-Dimethyl-6-nitroisobenzofuran-1(3H)-one

To a stirred solution of 3,3-dimethylisobenzofuran-1(3H)-one (8.72 g, 54 mmol) in cc. H₂SO₄ (100 mL) in a 250-mL round-bottom flask was added KNO₃ (8.0 g, 79 mmol) in portions at 0° C. The resulting solution was stirred for 2 h at RT. The reaction solution was poured into 1 L of water/ice. The isolated solid was collected. This resulted in 10.3 g (92%) of the title compound as a solid. MS-ESI: 208 (M+1).

### Step 3: 6-Amino-3,3-dimethylisobenzofuran-1(3H)-one

To a stirred solution of 3,3-dimethyl-6-nitroisobenzofuran-1(3H)-one (23 g, 111 mmol) in MeOH (200 mL) in a 500-mL round-bottom flask under nitrogen was added Pd/C (10% wt., 3.54 g) in portions at 0° C. The flask was evacuated and refilled three times with hydrogen. The resulting solution was stirred for 16 h at RT under atmosphere of hydrogen with a balloon. The solids were filtered out. The resulting mixture was concentrated under vacuum. The residue was eluted from silica gel with DCM/MeOH (40:1). This resulted in 18 g (91%) of the title compound as a light yellow solid. MS-ESI: 178 (M+1).

### Step 4: 2-(4-Amino-2-(hydroxymethyl)phenyl)propan-2-ol

To a stirred solution of 6-amino-3,3-dimethylisobenzofuran-1(3H)-one (5.8 g, 33 mmol) in THF (35 mL) in a 100-mL 3-necked round-bottom flask under nitrogen was added LiAlH₄ (2.5 g, 66 mmol) in portions at 0° C. The resulting solution was stirred for 16 h at RT. The resulting solution was quenched with 8.0 mL of MeOH. The mixture was filtered. The resulting mixture was concentrated under vacuum. The residue was eluted from silica gel with DCM/MeOH (20:1). This resulted in 5.3 g (89%) of the title compound as a brown yellow solid. MS-ESI: 180 (M+1).

### Step 5: 1,1-Dimethyl-1,3-dihydroisobenzofuran-5-amine

To a stirred solution of 2-(4-amino-2-(hydroxymethyl)phenyl)propan-2-ol (5.0 g, 27.6 mmol) in toluene (30 mL) in a 250-mL round-bottom flask was added H₃PO₄ (85% wt., 38 mL, 552 mmol) dropwise 0° C. The resulting solution was stirred for 3 h at 80° C. in an oil bath. The resulting solution was extracted with 60 mL of EtOAc and the aqueous layers were combined. The pH value of the aqueous layers was adjusted to 8 with NaOH (1 M). The resulting solution was extracted with 3×60 mL of EtOAc and the organic layers were combined and dried over anhydrous sodium sulfate and concentrated under vacuum. This resulted in 4.27 g (95%) of the title compound as an off-white solid. MS-ESI: 164 (M+1).

Steps 6-8 used similar procedures for converting compound 331′ to compound 334′ shown in Scheme 58 to afford compound 409′ from compound 406′. MS-ESI: 248 (M+1).

### Step 9: 5-Bromo-4,6-diisopropyl-1,1-dimethyl-1,3-dihydroisobenzofuran

To a stirred solution of 4,6-diisopropyl-1,1-dimethyl-1,3-dihydroisobenzofuran-5-amine (1.2 g, 4.86 mmol) in THF (15 mL) in a 50-mL 3-necked round-bottom flask under nitrogen was added 4A molecular sieve (5.0 g, powder) at RT followed by the addition of CuBr (3.49 g, 24.3 mmol) in portions at 0° C. To the solution was added LiBr (2.11 g, 24.3 mmol) in portions at RT. The solution was warmed to 70° C. in an oil bath. To the solution was added tert-butyl nitrite (1.0 g, 9.7 mmol) dropwise with stirring at 70° C. The resulting solution was stirred for 2 h at 70° C. in an oil bath. The reaction was then cooled to RT and diluted with 50 mL of PE. The solution was filtered and the filtrate washed with 3×50 mL of brine. The organic layer was dried over anhydrous sodium sulfate and concentrated under vacuum. The residue was eluted from silica gel column PE. This resulted in 960 mg (64%) of the title compound as a brown solid. MS-ESI: 311/313 (M+1).

Steps 10-11 used similar procedures for converting compound 335′ to intermediate 80′ shown in Scheme 58 to afford intermediate 69′ from compound 410′. MS-ESI: 289 (M−1).

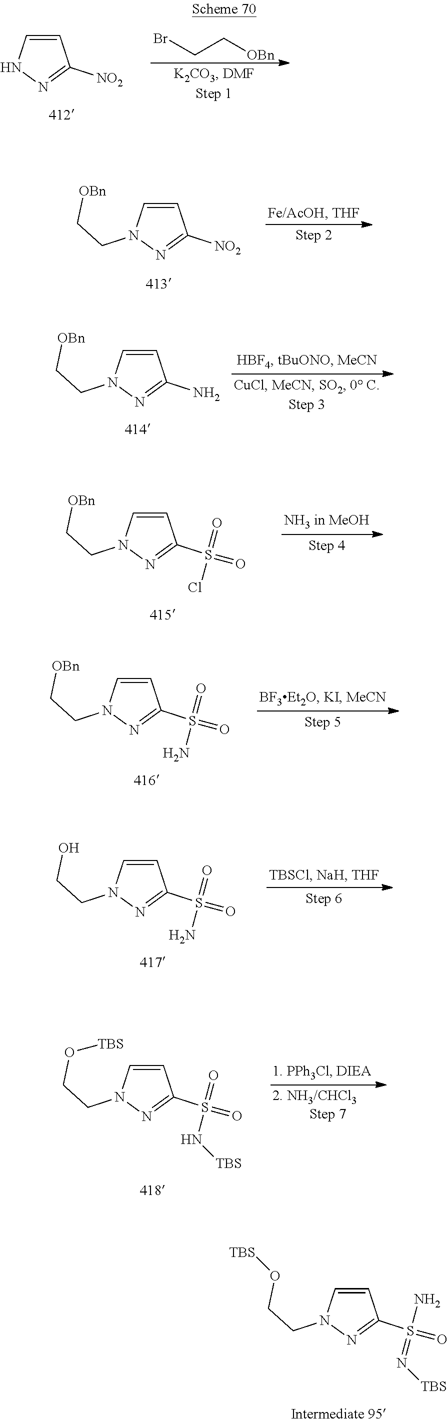

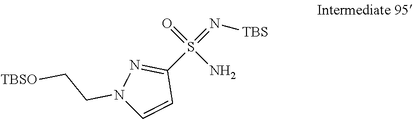

### N′-(tert-butyldimethylsilyl)-1-(2-((tert-butyldimethylsilyl)oxy)ethyl)-1H-pyrazole-3-sulfonimidamide

### Step 1: 1-(2-(Benzyloxy)ethyl)-3-nitro-1H-pyrazole

To a stirred solution of 3-nitro-1H-pyrazole (10 g, 88 mmol) in DMF (120 mL) were added K₂CO₃ (18 g, 133 mmol) in portions at RT, followed by the addition of ((2-bromoethoxy)methyl)benzene (20 g, 93 mmol) in DMF (10 mL) dropwise at RT. The reaction mixture was stirred for 16 h at 60° C. The reaction mixture was quenched with 150 mL of water. The mixture was extracted with 3×200 mL of EtOAc. The organic layers were combined and dried over anhydrous Na₂SO₄ and concentrated under vacuum. The crude product was eluted from silica gel with EtOAc/PE (1:3). This resulted in 21.1 g (96.7%) of the title compound as yellow oil. MS-ESI: 248 (M+1).

### Step 2: 1-(2-(Benzyloxy)ethyl)-1H-pyrazol-3-amine

To a stirred solution of 1-(2-(benzyloxy)ethyl)-3-nitro-1H-pyrazole (20 g, 81 mmol) in THF (200 mL) and AcOH (50 mL) was added Fe powder (45 g, 810 mmol) in portios at RT. The reaction mixture was stirred for 4 h at RT under nitrogen. The solids were filtered out, the filtrate was diluted with 400 mL of water and extracted with 3×300 mL of EtOAc, the organic layers were combined and dried over anhydrous Na₂SO₄ and concentrated under vacuum. The residue was eluted from silica gel with EtOAc/PE (3:1). This resulted in 14.8 g (84%) of the title compound as pink oil. MS-ESI: 218 (M+1).

### Step 3: 1-(2-(Benzyloxy)ethyl)-1H-pyrazole-3-sulfonyl Chloride

To a stirred solution of 1-(2-(benzyloxy)ethyl)-1H-pyrazol-3-amine (10 g, 46 mmol) in ACN (100 ml) were added aq. HBF₄ (40% wt., 15 g, 69.1 mmol) at RT, followed by the addition of tert-butyl nitrite (7.12 g, 69 mmol) dropwise below 5° C. The reaction solution was stirred for 1.5 h at 0˜5° C., this solution was assigned as solution A. Then CuCl (13.7 g, 138 mmol) was added to a 500-mL single necked round-bottom flask with ACN (200 mL) and SO₂ (g) was bubbled to the mixture with stirring at RT for 20 min, this mixture was assigned as mixture B. To the mixture B was added solution A dropwise with stirring at 0° C. The reaction mixture was stirred for additional 3 h at RT. The reaction was quenched with 500 mL of water. The mixture was extracted with 3×300 mL of EtOAc. The organic layers were combined and washed with 3×300 mL of H₂O. The organic layer was dried over anhydrous Na₂SO₄ and concentrated under vacuum. This resulted in 12.8 g (crude) of the title compound as brown yellow oil. MS-ESI: 301 (M+1).

### Step 4: 1-(2-(Benzyloxy)ethyl)-1H-pyrazole-3-sulfonamide

The solution of 1-(2-(benzyloxy)ethyl)-1H-pyrazole-3-sulfonyl chloride (crude from last step, 12.8 g) in NH₃ in MeOH (7 M, 300 mL) was stirred for 16 h at RT. The reaction mixture was concentrated under vacuum. The residue was eluted from silica gel with DCM/MeOH (20:1). This resulted in 5.4 g (42%, over two steps) of the title compound as brown yellow oil. MS-ESI: 282 (M+1).

### Step 5: 1-(2-Hydroxyethyl)-1H-pyrazole-3-sulfonamide

To a stirred solution of 1-(2-(benzyloxy)ethyl)-1H-pyrazole-3-sulfonamide (5.4 g, 19.2 mmol) in ACN (100 mL) under nitrogen was added KI (6.37 g, 38 mmol) in portions at RT. To the reaction mixture was added BF₃.Et₂O (47% wt., 13 g, 192 mmol) dropwise at RT. The reaction mixture was stirred for 4 h at RT. The reaction was quenched with 5.0 mL of water. The solids were filtered out. The filtrate was concentrated under vacuum. The residue was eluted from silica gel with DCM/MeOH (15:1). This resulted in 4.6 g (93%) of the title compound as a yellow solid. MS-ESI: 192 (M+1).

### Step 6: N-(tert-butyldimethylsilyl)-1-(2-((tert-butyldimethylsilyl)oxy)ethyl)-1H-pyrazole-3-sulfonamide

To a stirred solution of 1-(2-hydroxyethyl)-1H-pyrazole-3-sulfonamide (4.1 g, 21 mmol) in THF (60 mL) under nitrogen was added NaH (60% wt. dispersion in mineral oil, 3.86 g, 96.5 mmol) at 0° C. The reaction mixture was stirred for 20 min at RT. To the stirred mixture was added TBSCl (13.6 g, 90 mmol) at 0° C. The reaction mixture was stirred for 5 h at RT. The reaction was quenched with 300 mL of water. The mixture was extracted with 3×150 mL of EtOAc. The combined organic layers were dried over anhydrous Na₂SO₄ and concentrated under vacuum. The residue was eluted from silica gel with EtOAc/PE (1:5). This resulted in 6.2 g (69%) of the title compound as an off-white solid. MS-ESI: 420 (M+1).

### Step 7: N′-(tert-butyldimethylsilyl)-1-(2-((tert-butyldimethylsilyl)oxy)ethyl)-1H-pyrazole-3-sulfonimidamide

To a stirred mixture of PPh₃Cl₂ (1.51 g, 3.57 mmol) in CHCl₃ (15 ml) under nitrogen was added DIEA (924 mg, 7.15 mmol) dropwise at 0° C. The reaction mixture was stirred for 20 min at 0° C. To the stirred mixture was added N-(tert-butyldimethylsilyl)-1-(2-((tert-butyldimethylsilyl)oxy)ethyl)-1H-pyrazole-3-sulfonamide (600 mg, 1.43 mmol) in CHCl₃ (5.0 ml) dropwise at 0° C. The reaction mixture was stirred for 2 h at 0° C. NH₃ (g) was bubbled into the reaction mixture for 15 min at 0° C. Then the mixture was stirred for another 2 h at RT. The reaction was quenched with 20 mL of H₂O. The mixture was extracted with 3×20 mL of DCM. The organic layers were dried over anhydrous Na₂SO₄ and concentrated under vacuum. The residue was eluted from silica gel with EtOAc/PE (1:3). This resulted in 320 mg (53%) of the title compound as a yellow solid. MS-ESI: 419 (M+1).

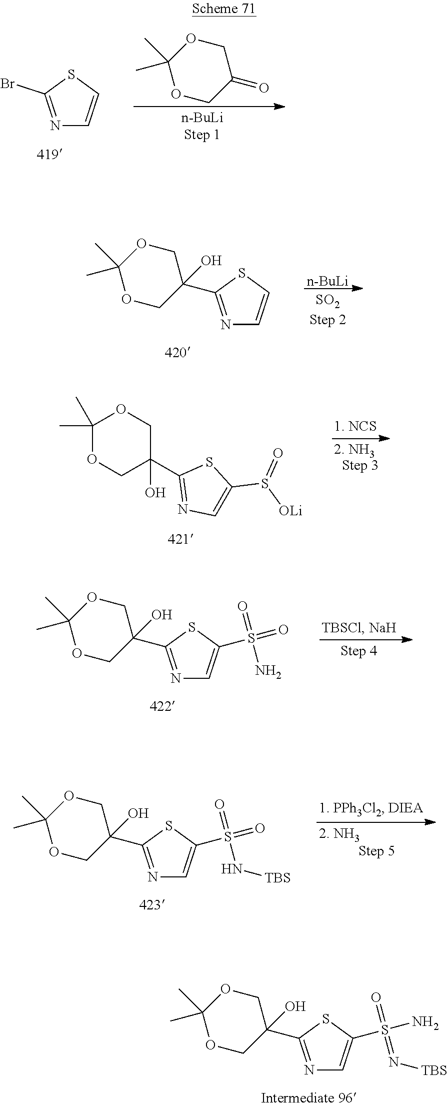

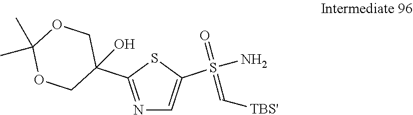

### N′-(tert-butyldimethylsilyl)-2-(5-hydroxy-2,2-dimethyl-1,3-dioxan-5-yl)thiazole-5-sulfonimidamide

### Step 1: 2,2-Dimethyl-5-(thiazol-2-yl)-1,3-dioxan-5-ol

To a stirred solution of 2-bromothiazole (4.89 g, 30 mmol) in THF (200 mL) under nitrogen was added n-BuLi in hexane (2.5 M, 12 mL, 30.0 mmol) dropwise at −78° C. The reaction solution was stirred for 30 min at −78° C. Then 2,2-dimethyl-1,3-dioxan-5-one (3.90 g, 30.0 mmol) in THF (10 mL) was added dropwise at −70° C. The reaction solution was stirred for an additional 30 min at RT. The reaction was quenched with 20 mL of MeOH and concentrated under vacuum. The residue was eluted from silica gel with EtOAc/PE (1/10). This resulted in 3.2 g (50%) of the title compound as a yellow solid. MS-ESI: 216 (M+1).

### Step 2: Lithium 2-(5-hydroxy-2,2-dimethyl-1,3-dioxan-5-yl)thiazole-5-sulfinate

To a stirred solution of 2,2-dimethyl-5-(thiazol-2-yl)-1,3-dioxan-5-ol (2.15 g, 10.0 mmol) in THF (100 mL) under nitrogen was n-BuLi in hexane (2.5 M, 4.0 mL, 10.0 mmol) dropwise with stirring at −70° C. The reaction solution was stirred for 60 min at −70° C. Then SO₂ (g) was bubbled to the solution at −50° C. for 10 min. The reaction mixture was stirred for an additional 30 min at 20° C. The reaction mixture was concentrated under vacuum. This resulted in 2.5 g (crude) of the title compound as an off-white solid. MS-ESI: 278 (M−1).

### Step 3: 2-(5-Hydroxy-2,2-dimethyl-1,3-dioxan-5-yl)thiazole-5-sulfonamide

To a stirred mixture of lithium 2-(5-hydroxy-2,2-dimethyl-1,3-dioxan-5-yl)thiazole-5-sulfinate (2.5 g, crude) in DCM (100 mL) was added NCS (2.67 g, 20.0 mmol) in small portions at RT. The reaction mixture was stirred for 2 h at RT. The reaction mixture was diluted with 50 mL of water, then extracted with 3×50 mL of DCM and the organic layers were combined and dried over anhydrous Na₂SO₄. Then NH₃ (g) was bubbled into the organic layer for 10 min at 0° C. The reaction mixture was stirred for 2 h at RT. The reaction mixture was concentrated under vacuum. The residue was eluted from silica gel with EtOAc/PE (1/3). This resulted in 1.8 g (61%, over two steps) of the title compound as a yellow solid. MS-ESI: 293 (M−1).

Steps 4-5 used similar procedures for converting compound 417′ to intermediate 95′ shown in Scheme 70 to afford intermediate 96′ from compound 422′. MS-ESI: 408 (M+1).

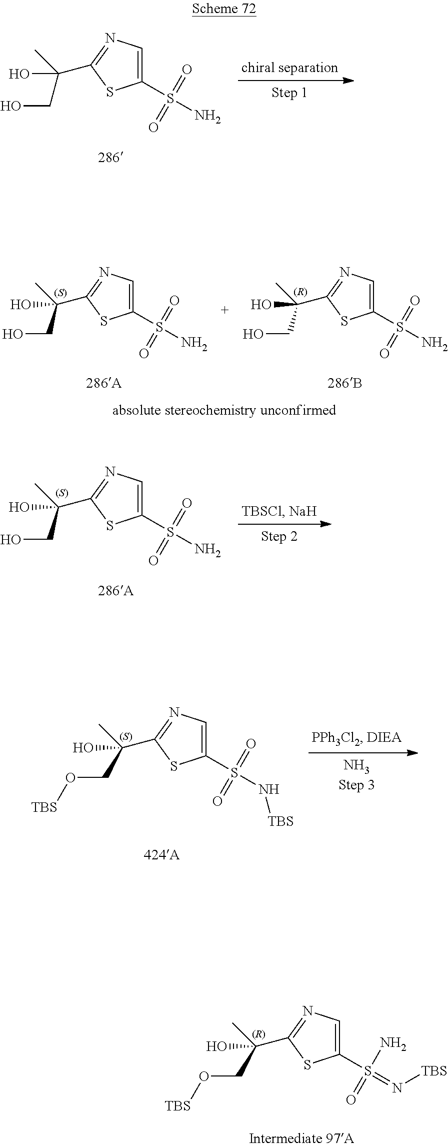

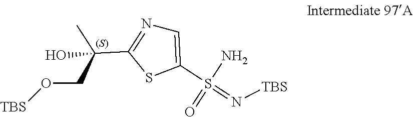

### N′-(tert-butyldimethylsilyl)-2-((S or R)-1-((tert-butyldimethylsilyl)oxy)-2-hydroxypropan-2-yl)thiazole-5-sulfonimidamide

### Step 1: (S) or (R)-2-(1,2-dihydroxypropan-2-yl)thiazole-5-sulfonamide

2-(1,2-Dihydroxypropan-2-yl)thiazole-5-sulfonamide (286′, 5.0 g) was resolved by prep-chiral HPLC using the following conditions: CHIRALPAK AD, 5*25 cm, 5 um; Mobile Phase A: CO₂, Mobile Phase B: MeOH:ACN=1:1 (2 mM NH₃-MeOH); Flow rate: 200 mL/min; Gradient: 40% B; UV 220 nm; Rt₁: 3.5 min (286′A); Rt₂: 5.6 min (286′B). This resulted in 2.0 g (99% ee) of 286′A and 2.1 g (98% ee) of 286′B, both as white solids. MS-ESI: 237 (M−1).

Steps 2-3 used similar procedures for converting compound 417′ to intermediate 95′ shown in Scheme 70 to afford intermediate 97′A from compound 286′A. MS-ESI: 466 (M+1).

TABLE 40

| | | | Exact |
|---|---|---|---|
| Intermediate | | | Mass |
| # | Structure | IUPAC Name | [M − H]⁻ |
| Intermediate 97′B | 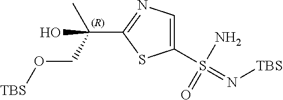 | N'-(tert-butyldimethylsilyl)-2-((R or S)-1-((tert- butyldimethylsilyl)oxy)-2-hydroxypropan-2-yl)thiazole-5- sulfonimidamide | 466 |

The intermediates in the following table were prepared using the similar procedures for converting compound 286′A to intermediate 97′A shown in Scheme 72 using compound 286′B.

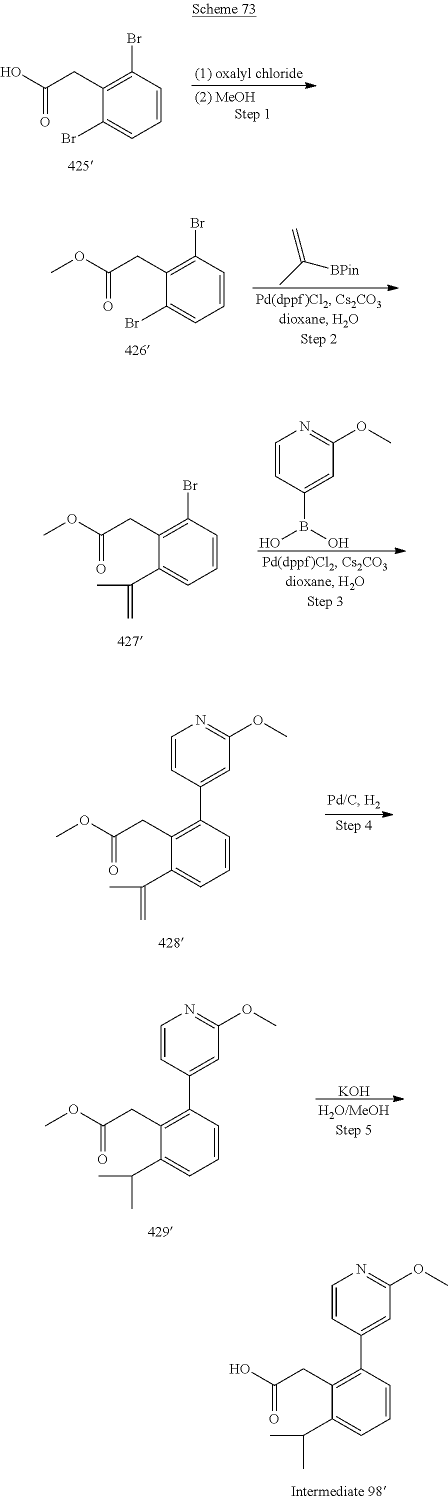

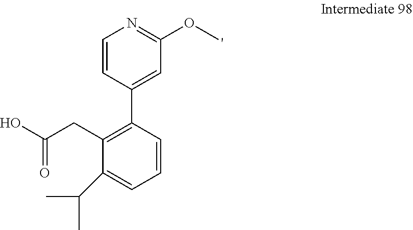 [SMILES: COc1cc(-c2cccc(C(C)C)c2CC(=O)O)ccn1]

### 2-(2-Isopropyl-6-(2-methoxypyridin-4-yl)phenyl)acetic Acid

### Step 1: Methyl 2-(2,6-dibromophenyl)acetate

To a stirred solution of 2-(2,6-dibromophenyl)acetic acid (10 g, 34 mmol) in DCM (100 mL) under nitrogen were added oxalyl chloride (6.5 g, 51 mmol) dropwise at RT, followed by the addition of DMF (0.26 mL, 3.4 mmol) dropwise at RT. The reaction solution was stirred for 0.5 h at RT. Then to the above solution was added MeOH (20 mL) dropwise at 0° C. and the reaction solution was stirred for additional 2 h at RT. The reaction solution was concentrated under reduced pressure. The residue was eluted from silica gel with PE/EtOAc (20:1). This resulted in 10 g (95%) of the title compound as colorless liquid. MS-ESI: 357/359/361 (M+1).

### Step 2: Methyl 2-(2-bromo-6-(prop-1-en-2-yl)phenyl)acetate

To a stirred solution of methyl 2-(2,6-dibromophenyl)acetate (13 g, 42 mmol) in dioxane (200 mL) and water (20 mL) under nitrogen were added 4,4,5,5-tetramethyl-2-(prop-1-en-2-yl)-1,3,2-dioxaborolane (7.09 g, 42 mmol), Cs₂CO₃ (41 g, 127 mmol) and Pd(dppf)Cl₂ (3.09 g, 4.22 mmol) in portions at RT. The reaction mixture was stirred for 3 h at 90° C. The reaction was quenched with water (200 mL) at RT. The mixture was extracted with DCM (3×300 mL). The combined organic layers were dried over anhydrous Na₂SO₄ and concentrated under vacuum. The residue was eluted from silica gel with PE/EtOAc (40:1). This resulted in 10 g (88%) of the title compound as light yellow liquid. MS-ESI: 269/271 (M+1).

### Step 3: Methyl 2-(2-(2-methoxypyridin-4-yl)-6-(prop-1-en-2-yl)phenyl)acetate

To a stirred solution of methyl 2-(2-bromo-6-(prop-1-en-2-yl)phenyl)acetate (9.4 g, 35 mmol) in dioxane (150 mL) and water (15 ml) under nitrogen were added (2-methoxypyridin-4-yl)boronic acid (5.34 g, 35 mmol), Cs₂CO₃ (34 g, 105 mmol) and Pd(dppf)Cl₂ (2.56 g, 3.49 mmol) in portions at RT. The reaction mixture was stirred for 3 h at 90° C., quenched with water (200 mL) at RT, and extracted with EtOAc (3×200 mL). The combined organic layers were dried over anhydrous Na₂SO₄ and concentrated under vacuum. The residue was eluted from silica gel with PE/EtOAc (10:1). This resulted in 6.6 g (64%) of the title compound as light yellow liquid. MS-ESI: 298 (M+1).

### Step 4: Methyl 2-(2-isopropyl-6-(2-methoxypyridin-4-yl)phenyl)acetate

To a stirred solution of methyl 2-(2-(2-methoxypyridin-4-yl)-6-(prop-1-en-2-yl)phenyl)acetate (6.6 g, 22 mmol) in MeOH (50 mL) under nitrogen was added Pd/C (10% wt., 1.44 g) in portions at 0° C. The flask was evacuated and refilled three times with hydrogen. The reaction mixture was stirred for 3 h at RT under atmosphere of hydrogen with a balloon. The reaction mixture was filtered, the filter cake was washed with MeOH (3×50 mL). The filtrate was concentrated under vacuum. This resulted in 6.5 g (98%) of the title compound as a colorless liquid. MS-ESI: 300 (M+1).

### Step 5: 2-(2-Isopropyl-6-(2-methoxypyridin-4-yl)phenyl)acetic Acid

To a stirred solution of methyl 2-(2-isopropyl-6-(2-methoxypyridin-4-yl)phenyl)acetate (6.5 g, 22 mmol) in MeOH (90 mL) and H₂O (30 mL) was added potassium hydroxide (4.87 g, 87 mmol) in portions at 0° C. The reaction mixture was stirred for 1 h at 90° C. The mixture was adjusted to pH=6˜7 with conc. HCl. The mixture was extracted with EtOAc (3×100 mL). The combined organic layers were dried over anhydrous Na₂SO₄ and concentrated under vacuum. The residue was eluted from silica gel with DCM/MeOH (20:1). This resulted in 5.3 g (86%) of the title compound as a light yellow solid. MS-ESI: 284 (M−1).

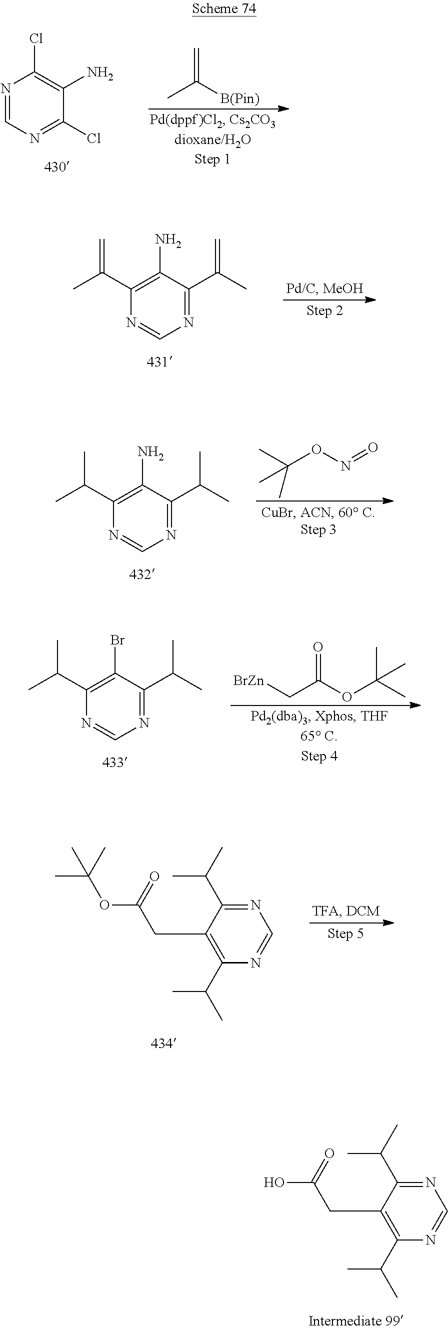

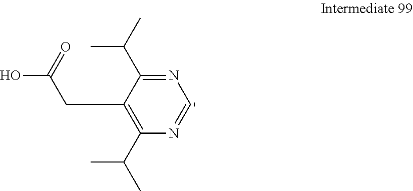 [SMILES: CC(C)c1ncnc(C(C)C)c1CC(=O)O]

### 2-(4,6-Diisopropylpyrimidin-5-yl)acetic Acid

### Step 1: 4,6-Di(prop-1-en-2-yl)pyrimidin-5-amine

To a stirred solution of 4,6-dichloropyrimidin-5-amine (10 g, 61 mmol) in dioxane (300 mL) and H₂O (30 mL) under nitrogen were added 4,4,5,5-tetramethyl-2-(prop-1-en-2-yl)-1,3,2-dioxaborolane (26 g, 152 mmol), Cs₂CO₃ (50 g, 152 mmol) and Pd(dppf)Cl₂ (2.23 g, 3.05 mmol) in portions at RT. The reaction mixture was stirred overnight at 80° C. under nitrogen. The reaction mixture was concentrated under vacuum, diluted with H₂O (200 mL), and extracted with EtOAc (3×200 mL). The combined organic layer was dried over anhydrous Na₂SO₄ and concentrated under vacuum. The residue was eluted from silica gel with EtOAc/PE (1:10). This resulted in 10 g (94%) of the title compound as yellow oil. MS-ESI: 176 (M+1).

Step 2 used similar procedures for converting compound 428′ to intermediate 429′ shown in Scheme 73 to afford compound 432′ from compound 431′. MS-ESI: 180 (M+1).

### Step 3: 5-Bromo-4,6-diisopropylpyrimidine

To a stirred solution of 4,6-diisopropylpyrimidin-5-amine (2.0 g, 11 mmol) in ACN (80 mL) under nitrogen were added CuBr (3.2 g, 22 mmol) and tert-butyl nitrite (2.3 g, 22 mmol) at 0° C. The reaction mixture was stirred at 0° C. for 10 min. The reaction mixture was allowed to react for an additional 2 h at 60° C. The reaction mixture was concentrated under vacuum. The residue was eluted from silica gel with EtOAc/PE (1:10). This resulted in 1.39 g (51%) of the title compound as yellow oil. MS-ESI: 243/245 (M+1).

### Step 4: Tert-butyl 2-(4,6-diisopropylpyrimidin-5-yl)acetate

To a stirred solution of 5-bromo-4,6-diisopropylpyrimidine (1.39 g, 5.7 mmol) in THF (50 mL) under nitrogen were added tert-butyl 2-(bromozincio)acetate (4.47 g, 17 mmol), Xphos (273 mg, 0.57 mmol) and Pd₂(dba)₃ (262 mg, 0.29 mmol). The reaction mixture was stirred for 2 h at 65° C. under nitrogen. The reaction mixture was concentrated under vacuum. The residue was eluted from silica gel with EtOAc/PE (1:20). This resulted in 1.5 g (94%) of the title compound as a purple solid. MS-ESI: 279 (M+1).

### Step 5: 2-(4,6-Diisopropylpyrimidin-5-yl)acetic Acid

To a stirred solution of tert-butyl 2-(4,6-diisopropylpyrimidin-5-yl)acetate (1.5 g, 5.4 mmol) in DCM (8.0 mL) was added TFA (8.0 mL) dropwise at RT. The reaction solution was stirred for 3 h at RT. The reaction mixture was concentrated under vacuum. The residue was eluted from silica gel with EtOAc/PE (1:1). This resulted in 1.1 g (93%) of the title compound as a light yellow solid. MS-ESI: 221 (M−1).

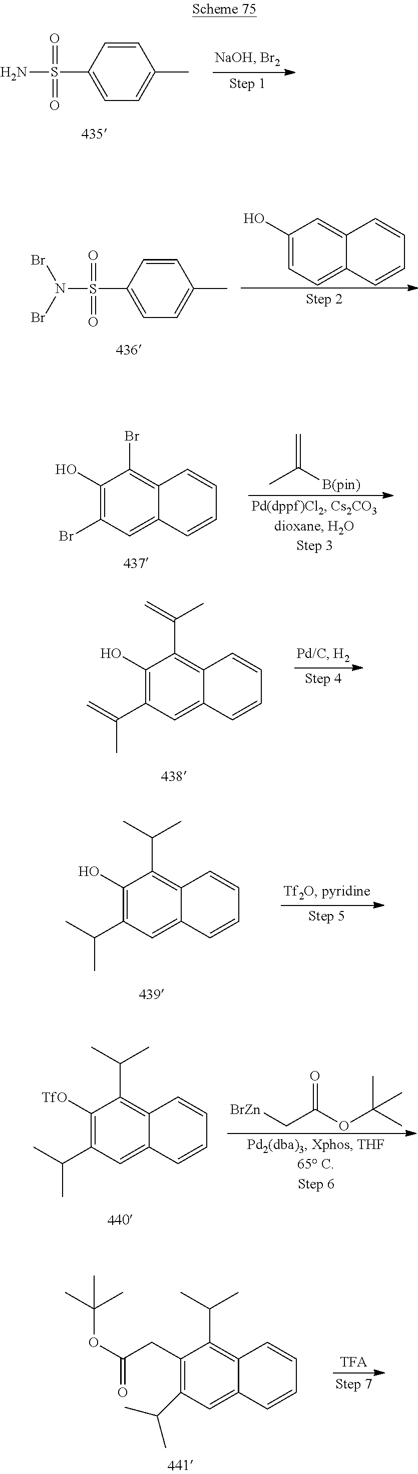

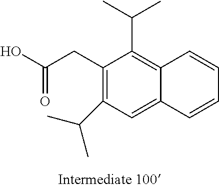 [SMILES: CC(C)c1cc2ccccc2c(C(C)C)c1CC(=O)O]

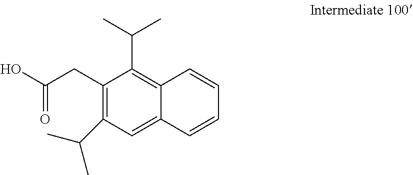 [SMILES: CC(C)c1cc2ccccc2c(C(C)C)c1CC(=O)O]

### 2-(1,3-Diisopropylnaphthalen-2-yl)acetic Acid

### Step 1: N,N-dibromo-4-methylbenzenesulfonamide

To a stirred solution of NaOH (3.71 g, 93 mmol) in water (75 mL) was added 4-methylbenzenesulfonamide (6.4 g, 37 mmol) at RT, followed by the addition of Br₂ (6.51 mL, 127 mmol) dropwise at 0° C. The reaction mixture was stirred for 12 h at RT. The reaction mixture was filtered and the filter cake was washed with cold water. The crude product was re-crystallized from EtOH. This resulted in 11 g (90%) of the title compound as a yellow solid. MS-ESI: 328/310/312 (M+1).

### Step 2: 1,3-Dibromonaphthalen-2-ol

To a stirred solution of naphthalen-2-ol (1.3 g, 9.0 mmol) in ACN (20 mL) was added N,N-dibromo-4-methylbenzenesulfonamide (2.96 g, 9.0 mmol) in portions at 0° C. The reaction solution was stirred for 3 h at RT. The reaction was quenched with sat. Na₂S₂O₃ (30 mL). The mixture was extracted with EtOAc (3×50 mL) and the organic layers were combined. The organic layer was dried over anhydrous Na₂SO₄ and concentrated under vacuum. The residue was eluted from silica gel with EtOAc/PE (1:4). This resulted in 1.35 g (50%) of the title compound as a white solid. MS-ESI: 301/303/305 (M+1).

Steps 3-4 used similar procedures for converting compound 430′ to compound 432′ shown in Scheme 74 to afford compound 439′ from compound 437′. MS-ESI: 229 (M+1).

### Step 5: 1,3-Diisopropylnaphthalen-2-yl Trifluoromethanesulfonate

To a stirred solution of 1,3-diisopropylnaphthalen-2-ol (1.1 g, 4.8 mmol) in DCM (50 mL) under nitrogen were added pyridine (572 mg, 7.23 mmol) and Tf₂O (1.6 g, 5.78 mmol) at 0° C. The reaction mixture was stirred for 5 h at RT. The reaction was quenched with HCl (aq.) (10% wt., 10 mL). The mixture was diluted with 50 mL of H₂O. The mixture was extracted with 3×50 mL of DCM. The combined organic layer was washed with 50 mL of sat. NaHCO₃ and 50 mL of brine. The organic layer was dried over anhydrous Na₂SO₄ and concentrated under vacuum. The residue was eluted from silica gel with PE. This resulted in 1.2 g (69%) of the title compound as yellow oil. MS-ESI: 361 (M+1).

Steps 6-7 used similar procedures for converting compound 433′ to intermediate 99′ shown in Scheme 74 to afford intermediate 100′ from compound 440′. MS-ESI: 269 (M−1).

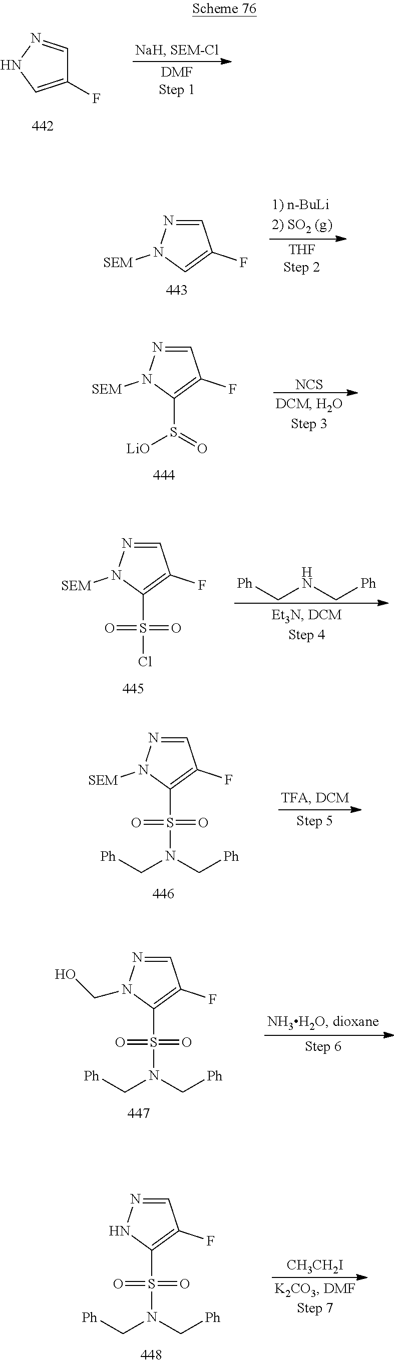

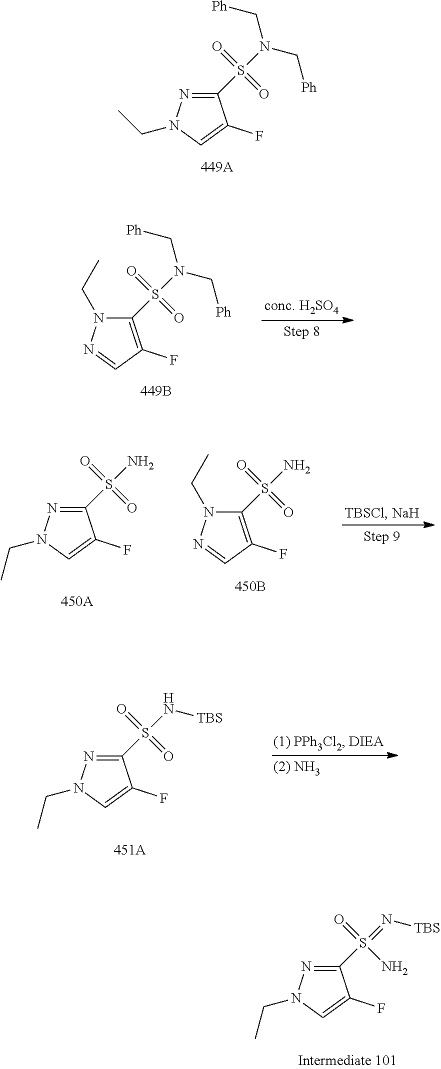

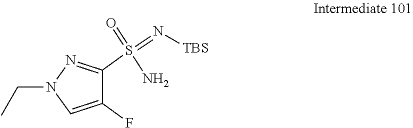

### N′-(tert-butyldimethylsilyl)-1-ethyl-4-fluoro-1H-pyrazole-3-sulfonimidamide

### Step 1: 4-Fluoro-1-((2-(trimethylsilyl)ethoxy)methyl)-1H-pyrazole

To a stirred solution of 4-fluoro-1H-pyrazole (5.0 g, 58 mmol) in DMF (53 mL) in a 250-mL 3-necked round-bottom flask under nitrogen was added NaH (60% wt. dispersion in mineral oil, 5.36 g, 134 mmol) in portions at 0° C. in an ice/water bath over 10 min. The resulting solution was stirred for 30 min at 10° C. To this was added SEM-Cl (22 g, 134 mmol) dropwise with stirring at 0° C. over 10 min. The resulting solution was stirred overnight at RT. The reaction was then quenched with 60 mL of water. The resulting solution was extracted with 60 mL of EtOAc. The combined organic layer was washed with 5×60 ml of sat. NaCl solution. The resulting mixture was concentrated. The residue was eluted from silica gel column EtOAc/PE (1:100). This resulted in 13.7 g (crude) of the title compound as a light yellow liquid. MS-ESI: 217 (M+1).

### Step 2: Lithium 4-fluoro-1-((2-(trimethylsilyl)ethoxy)methyl)-1H-pyrazole-5-sulfinate

To a stirred solution of 4-fluoro-1-((2-(trimethylsilyl)ethoxy)methyl)-1H-pyrazole (13.7 g, 63 mmol) in THF (150 mL) in a 500-mL 3-necked round-bottom flask under nitrogen was added n-BuLi in hexane (2.5 M, 28 mL, 70 mmol) dropwise at −78° C. over 15 min. The resulting solution was stirred for 1 h at −78° C. Then to the mixture was introduced SO₂ (g) bubble for 20 min −78° C. The resulting solution was stirred for 1 h at RT. The resulting mixture was concentrated. This resulted in 20.4 g (crude) of the title compound as a white solid. MS-ESI: 279 (M−1).

### Step 3: 4-Fluoro-1-((2-(trimethylsilyl)ethoxy)methyl)-1H-pyrazole-5-sulfonyl Chloride

To a stirred solution of lithium 4-fluoro-1-((2-(trimethylsilyl)ethoxy)methyl)-1H-pyrazole-5-sulfinate (20.4 g, crude from last step) in DCM (396 mL) and H₂O (198 mL) was added NCS (10 g, 78 mmol) in portions at 0° C. The resulting solution was stirred for 1 h at 10° C. The crude product was used directly without work-up.

### Step 4: N,N-dibenzyl-4-fluoro-1-((2-(trimethylsilyl)ethoxy)methyl)-1H-pyrazole-5-sulfonamide

To the stirred solution of 4-fluoro-1-((2-(trimethylsilyl)ethoxy)methyl)-1H-pyrazole-5-sulfonyl chloride in DCM (396 mL) and H₂O (198 mL) from last step was added Et₃N (8.85 g, 87 mmol) and dibenzylamine (17 g, 84 mmol) dropwise at 0° C. The resulting solution was stirred for 1 h at 8° C. The reaction was then quenched by the addition of 300 mL of water. The resulting solution was extracted with 3×300 mL of DCM. The organic layers were combined and washed with brine (300 mL) and dried over anhydrous Na₂SO₄ and concentrated under vacuum. The residue was eluted from silica gel with EtOAc/PE (1:19). This resulted in 22.5 g (81% over 4 steps) of the title compound as light yellow oil. MS-ESI: 476 (M+1).

### Step 5: N,N-dibenzyl-4-fluoro-1-(hydroxymethyl)-1H-pyrazole-5-sulfonamide

To a stirred solution of N,N-dibenzyl-4-fluoro-1-((2-(trimethylsilyl)ethoxy)methyl)-1H-pyrazole-5-sulfonamide (22.5 g, 47 mmol) in DCM (25 mL) in a 250-mL round-bottom flask was added TFA (25 mL). The resulting solution was stirred 16 h at RT. The resulting mixture was concentrated under vacuum. The residue was eluted from silica gel with EtOAc/PE (1:4). This resulted in 15 g (85%) of the title compound as yellow oil. MS-ESI: 376 (M+1).

### Step 6: N,N-dibenzyl-4-fluoro-1H-pyrazole-5-sulfonamide

To a stirred solution of N,N-dibenzyl-4-fluoro-1-(hydroxymethyl)-1H-pyrazole-5-sulfonamide (15 g, 40 mmol) in dioxane (50 mL) in a 500-mL round-bottom flask was added NH₃.H₂O (30% wt., 50 mL) dropwise at 0° C. The resulting solution was stirred for 3 h at RT. The resulting mixture was concentrated. The residue was eluted from silica gel with EtOAc/PE (1:1). This resulted in 12 g (87%) of the title compound as a white solid. MS-ESI: 346 (M+1).

### Step 7: N,N-dibenzyl-1-ethyl-4-fluoro-1H-pyrazole-3-sulfonamide and N,N-dibenzyl-1-ethyl-4-fluoro-1H-pyrazole-5-sulfonamide

To a stirred solution of N,N-dibenzyl-4-fluoro-1H-pyrazole-5-sulfonamide (1.1 g, 3.2 mmol) in DMF (20 mL) in a 100-mL 3-necked round-bottom flask under nitrogen was added K₂CO₃ (0.88 g, 6.4 mmol) in portions at RT and ethyl iodide (0.99 g, 6.4 mmol) dropwise at RT. The resulting solution was stirred for 3 h at 110° C. The reaction was then quenched by the addition of 20 mL of water. The resulting solution was extracted with 2×20 mL of EtOAc. The organic layers were dried over anhydrous Na₂SO₄ and concentrated. The residue was eluted from silica gel with EtOAc/PE (3:17). This resulted in 764 mg (64%) of 458A and 218 mg (18%) of 458B both as a light yellow solid. MS-ESI: 374 (M+1).

### Step 8: 1-Ethyl-4-fluoro-1H-pyrazole-3-sulfonamide and 1-ethyl-4-fluoro-1H-pyrazole-5-sulfonamide

To a stirred solution of N,N-dibenzyl-1-ethyl-4-fluoro-1H-pyrazole-3-sulfonamide (764 mg, 2.0 mmol) in DCM (1.5 mL) in a 25-mL round-bottom flask was added H₂SO₄ (98% wt., 3.00 mL) dropwise at 0° C. The resulting solution was stirred for 1 h at RT. The reaction was then quenched by the addition of 5.0 mL of water/ice. The mixture was extracted with EtOAc (3×50 mL). The organic layer was dried over anhydrous Na₂SO₄ and concentrated under reduced vacuum. The residue was eluted from silica gel with DCM/MeOH (93:7). This resulted in 317 mg (80%) of the title compound as a white solid. MS-ESI: 194 (M+1). ¹H NMR (300 MHz, DMSO-d₆) δ 8.08 (d, J=4.7 Hz, 1H), 7.77 (s, 2H), 4.14 (q, J=7.3 Hz, 2H), 1.39 (t, J=7.3 Hz, 3H). The structure was confirmed by NOESY: Ar—H 8.08 (d, J=4.7 Hz, 1H) has correlations with CH₂ from Et at 4.14 (q, J=7.3 Hz, 2H) and CH₃ from Et at 1.39 (t, J=7.3 Hz, 3H)

Similar procedure was used for converting compound 449A to compound 450A shown in Scheme 76 to afford compound 450B from compound 449B. MS-ESI: 194 (M+1). ¹H NMR (400 MHz, DMSO-d₆) δ 8.20 (s, 2H), 7.67 (d, J=4.5 Hz, 1H), 4.33 (q, J=7.2 Hz, 2H), 1.35 (t, J=7.2 Hz, 3H). The structure was confirmed by NOESY: NH₂ at 8.20 (s, 2H) has correlations with CH₂ from Et at 4.33 (q, J=7.2 Hz, 2H) and CH₃ from Et at 1.35 (t, J=7.2 Hz, 3H)

Steps 9-10 used similar procedures for converting compound 417′ to intermediate 95′ shown in Scheme 70 to afford Intermediate 101 from compound 450A. MS-ESI: 307 (M+1).

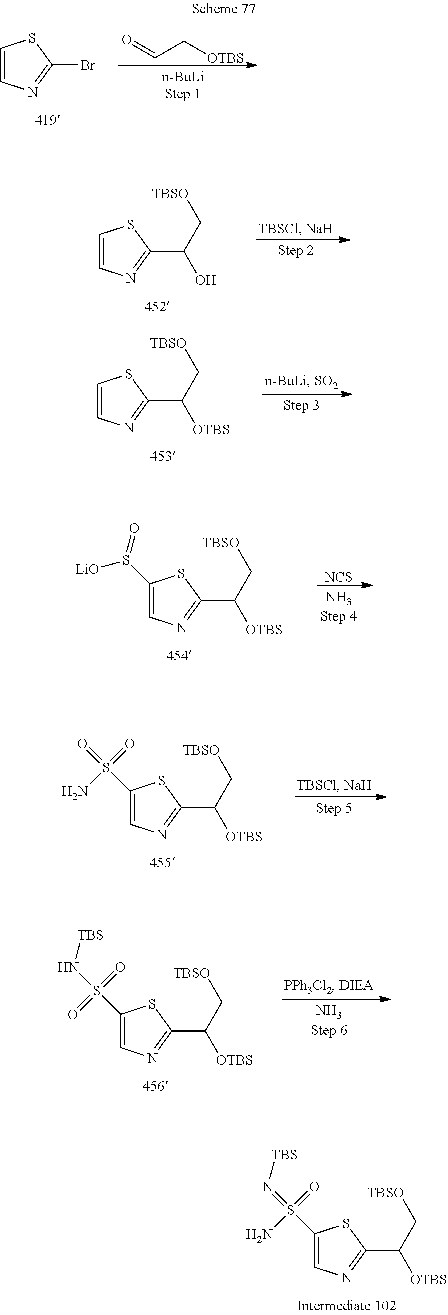

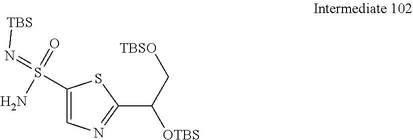

### N′-(tert-butyldimethylsilyl)-2-(2,2,3,3,8,8,9,9-octamethyl-4,7-dioxa-3,8-disiladecan-5-yl)thiazole-5-sulfonimidamide

### Step 1: 2-((Tert-butyldimethylsilyl)oxy)-1-(thiazol-2-yl)ethan-1-ol

To a stirred solution of 2-bromothiazole (10.0 g, 61.3 mmol) in THF (120 mL) under nitrogen was added n-BuLi (2.5 M in hexane, 36.8 mL, 92.0 mmol) dropwise at −78° C. The reaction mixture was stirred at −78° C. for 20 min. Then to the above solution was added 2-((tert-butyldimethylsilyl)oxy)acetaldehyde (16.0 g, 92.0 mmol) in THF (10 mL) dropwise at −78° C. The resulting mixture was stirred for 2 h at −50° C. The reaction was quenched with ice/water (50 mL), the resulting mixture was concentrated to remove THF under vacuum. The aqueous phase was extracted with EtOAc (3×100 mL). The organic layers were combined, dried over anhydrous Na₂SO₄, and concentrated under vacuum. The residue was eluted from silica gel with EtOAc/PE (1:9). This resulted in 8.1 g (51%) of the title compound as yellow oil. MS-ESI: 260 (M+1).

### Step 2: 2-(2,2,3,3,8,8,9,9-Octamethyl-4,7-dioxa-3,8-disiladecan-5-yl)thiazole

To a stirred solution of 2-((tert-butyldimethylsilyl)oxy)-1-(thiazol-2-yl)ethan-1-ol (8.0 g, 30.9 mmol) in THF (150 mL) under nitrogen was added NaH (60% wt dispersion in mineral oil, 3.09 g, 77.3 mmol) in portions at 0° C. The resulting mixture was stirred for 10 min at 0° C. Then to the above mixture was added TBSCl (18.6 g, 124 mmol) in THF (15 mL) dropwise at 0° C. The resulting mixture was stirred for 16 h at RT. The reaction was quenched with ice/water (100 mL), the resulting mixture was concentrated to remove THF under vacuum. The aqueous phase was extracted with EtOAc (3×100 mL). The organic layers were combined, dried over anhydrous Na₂SO₄, and concentrated under vacuum. The residue was eluted from silica gel with EtOAc/PE (4:96). This resulted in 8 g (69%) of the title compound as yellow oil. MS-ESI: 374 (M+1).

Steps 3-6 used similar procedures for converting compound 420′ to Intermediate 96′ shown in Scheme 71 to afford Intermediate 102 from compound 453′. MS-ESI: 566 (M+1).

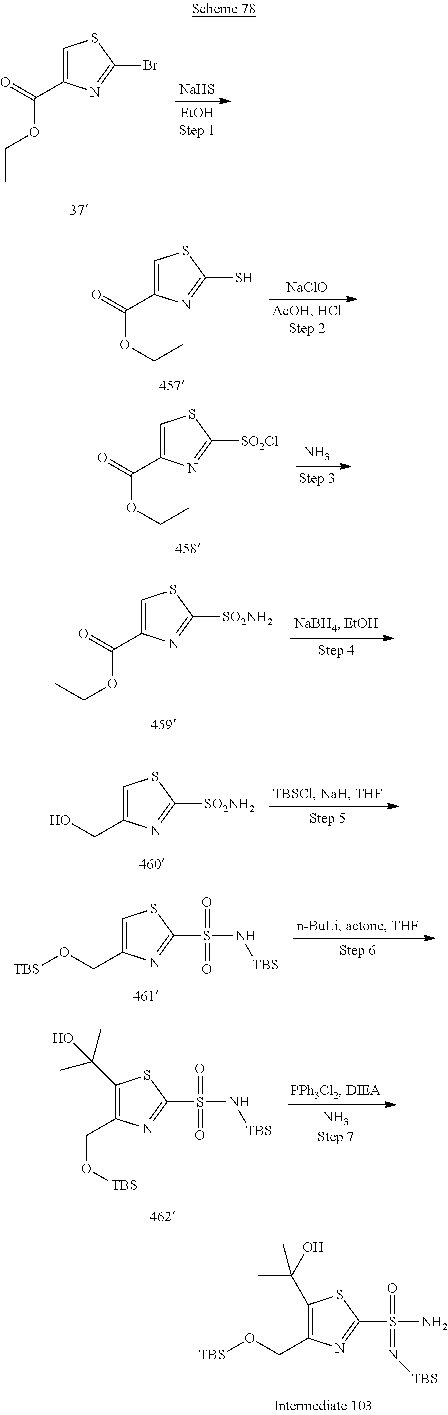

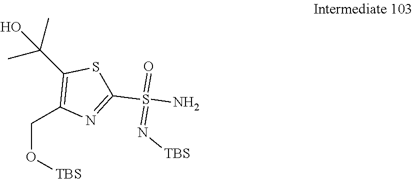

### N′-(tert-butyldimethylsilyl)-4-(((tert-butyldimethylsilyl)oxy)methyl)-5-(2-hydroxypropan-2-yl)thiazole-2-sulfonimidamide

### Step 1: Ethyl 2-mercaptothiazole-4-carboxylate

To a stirred solution of ethyl 2-bromothiazole-4-carboxylate (23.6 g, 100 mmol) in EtOH (300 mL) under nitrogen was added NaSH (11.2 g, 200 mmol) in portions at RT. The resulting solution was stirred for 3 h at 85° C. The resulting mixture was concentrated under vacuum. The residue was dissolved in 600 mL of water. The pH value of the solution was adjusted to 3 with aq. HCl (1 M). The solids were collected by filtration and dried under an infrared lamp. This resulted in 18.0 g (95.1%) of the title compound as a yellow solid. MS-ESI. 190 (M+1).

### Step 2: Ethyl 2-(chlorosulfonyl)thiazole-4-carboxylate

To a stirred solution of ethyl 2-mercaptothiazole-4-carboxylate (16.0 g, 84.7 mmol) in aq. HCl (6 M, 100 mL) and AcOH (100 mL) was added aq. NaClO (8%-10% chlorine, 150 mL) dropwise at 0° C. The resulting solution was stirred for 2 h at 0° C. The residue was diluted with 200 mL of water and extracted with 2×200 mL of DCM. The organic layers were combined and washed with brine (3×200 mL). The organic phase was dried over anhydrous Na₂SO₄, then the filtrate was collected by filtration. This resulted in the title compound in a DCM solution which was used in next step without further purification.

### Step 3: Ethyl 2-sulfamoylthiazole-4-carboxylate

NH₃ (g) was bubbled in DCM (1000 mL) with stirring at 0° C. for 30 min, then to this solution was added ethyl 2-(chlorosulfonyl)thiazole-4-carboxylate in DCM (400 mL, crude from last step) dropwise at 0° C. over 30 min. The resulting solution was warm to RT and stirred for 1 h at RT. The reaction mixture was quenched with 800 mL of water and the organic layer was collected and dried over anhydrous Na₂SO₄ and concentrated under vacuum. The residue was re-crystallized from MeOH. This resulted in 4.94 g (24.7% over 2 steps) of the title compound as an off-white solid. MS-ESI: 235 (M−1).

### Step 4: 4-(Hydroxymethyl)thiazole-2-sulfonamide

To a stirred solution of ethyl 2-sulfamoylthiazole-4-carboxylate (4.94 g, 20.9 mmol) in EtOH (50 mL) was added NaBH₄ (6.35 g, 167 mmol) in portions at 0° C. The resulting solution was stirred for 4 h at 0° C. The reaction was then quenched with 100 mL of water/ice. The pH value of the solution was adjusted to 3 with cc. HCl. The resulting mixture was concentrated. The residue was eluted from silica gel with EtOAc. This resulted in 2.80 g (69.1%) of the title compound as an off-white solid. MS-ESI: 193 (M−1).

### Step 5: N-(tert-butyldimethylsilyl)-4-(((tert-butyldimethylsilyl)oxy)methyl)thiazole-2-sulfonamide

To a stirred solution of 4-(hydroxymethyl)thiazole-2-sulfonamide (2.80 g, 14.4 mmol) in THF (30 mL) under nitrogen was added NaH (60% wt, 4.61 g, 115 mmol) in several batches at 0° C. over 30 min, followed by TBSCl (6.48 g, 43.2 mmol) in THF (10 mL) dropwise at 0° C. The resulting solution was stirred for 1 h at RT. The reaction was quenched with water/ice (20 mL). The resulting mixture was diluted with 100 mL of water and extracted with EtOAc (200 mL). The organic layer was collected and washed with brine (200 mL), then dried over anhydrous Na₂SO₄ and concentrated under vacuum. The residue was eluted from silica gel with PE/EA (10:1). This resulted in 1.71 g (28.1%) of the title compound as yellow oil. MS-ESI: 421 (M−1).

### Step 6: N-(tert-butyldimethylsilyl)-4-(((tert-butyldimethylsilyl)oxy)methyl)-5-(2-hydroxypropan-2-yl)-thiazole-2-sulfonamide

To a stirred solution of N-(tert-butyldimethylsilyl)-4-(((tert-butyldimethylsilyl)oxy)methyl)thiazole-2-sulfonamide (1.71 g, 4.05 mmol) in dry THF (50 mL) under nitrogen was added n-BuLi (2.5 M in hexane, 16.2 mL, 40.5 mmol) dropwise at −78° C. The reaction mixture was stirred at −78° C. for 30 min. Then to the above mixture was added acetone (3.57 g, 61.6 mmol) dropwise at −78° C. The reaction mixture was stirred at −78° C. for 1.5 h. The reaction was quenched with water (100 mL). The mixture was extracted with ether (3×150 mL), the organic layers were combined, dried over anhydrous Na₂SO₄, and concentrated under vacuum. The residue was eluted from silica gel with PE/EtOAc (5:1). This resulted in 877 mg (45.1%) of the title compound as yellow solid. MS-ESI: 479 (M−1).

Steps 7 used similar procedures for converting compound 418′ to Intermediate 95 shown in Scheme 70 to afford Intermediate 103 from compound 462′. MS-ESI: 480 (M+1).

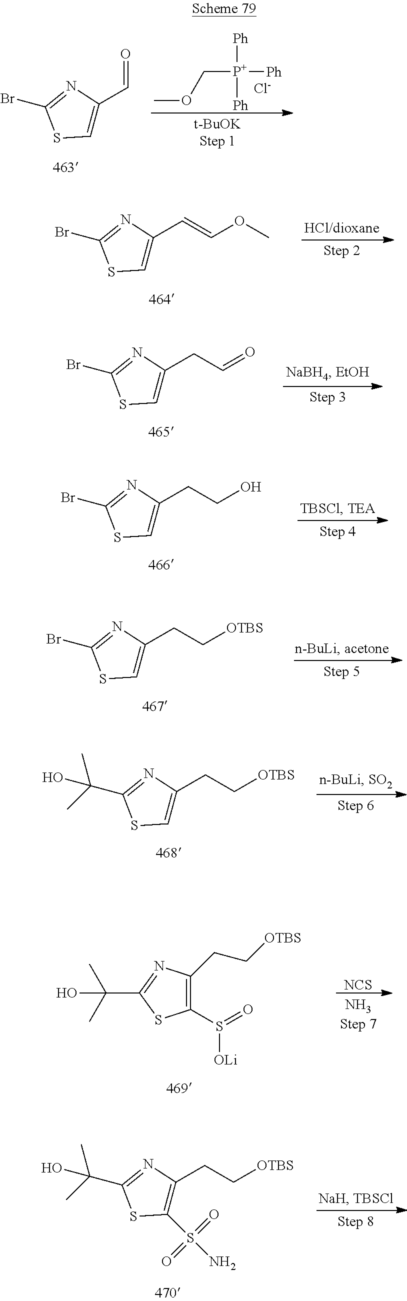

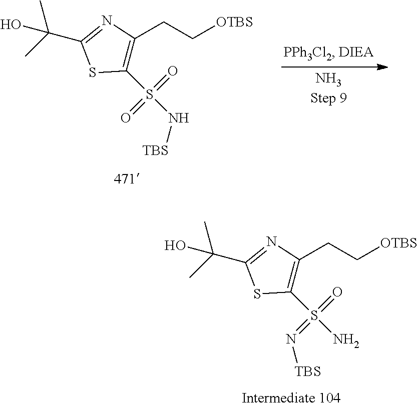

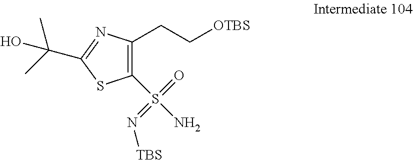

### N′-(tert-butyldimethylsilyl)-4-(2-((tert-butyldimethylsilyl)oxy)ethyl)-2-(2-hydroxypropan-2-yl)thiazole-5-sulfonimidamide

### Step 1: (E)-2-Bromo-4-(2-methoxyvinyl)thiazole

To a stirred solution of (methoxymethyl)triphenylphosphonium chloride (53.6 g, 157 mmol) in THF (500 mL) under nitrogen was added t-BuOK (17.5 g, 156 mmol) in portions at 0° C., followed by 2-bromothiazole-4-carbaldehyde (20.0 g, 105 mmol) in portions at 0° C. The resulting mixture was stirred for 16 h at RT. The reaction was quenched with water/ice (500 mL) and extracted with EtOAc (3×500 mL). The organic layers were combined, dried over anhydrous Na₂SO₄, and concentrated under vacuum. The residue was eluted from silica gel with EtOAc/PE (1:9). This resulted in 20.5 g (89.1%) of the title compound as brown oil. MS-ESI: 220/222 (M+1).

### Step 2: 2-(2-Bromothiazol-4-yl)acetaldehyde

To a stirred solution of (E)-2-bromo-4-(2-methoxyvinyl)thiazole (20.0 g, 91.3 mmol) in 1,4-dioxane (90 mL) was added HCl (4 M, 90 mL) dropwise at 0° C. The resulting solution was stirred for 3 h at RT. The reaction mixture was concentrated to remove dioxane under vacuum, then extracted with EtOAc (3×100 mL). The organic layers were combined, dried over anhydrous Na₂SO₄, and concentrated under vacuum. This resulted in 16.2 g (86.5%) of the title compound as yellow oil. MS-ESI: 206/208 (M+1).

### Step 3: 2-(2-Bromothiazol-4-yl)ethan-1-ol

To a stirred solution of 2-(2-bromothiazol-4-yl)acetaldehyde (4.0 g, 19.5 mmol) in EtOH (80 mL) was added NaBH₄ (1.10 g, 28.9 mmol) in portions at 0° C. The resulting solution was stirred for 2 h at 0° C. The resulting mixture was quenched with water (50 mL), then concentrated under vacuum to remove EtOH. The residue was extracted with EtOAc (2×100 mL). The organic layers were combined, washed with brine (100 mL), dried over anhydrous Na₂SO₄, and concentrated under vacuum. The residue was eluted from silica gel with EtOAc/PE (3:7). This resulted in 3.8 g (94%) of the title compound as yellow oil. MS-ESI: 208/210 (M+1).

### Step 4: 2-Bromo-4-(2-((tert-butyldimethylsilyl)oxy)ethyl)thiazole

To a stirred solution of 2-(2-bromo-1,3-thiazol-4-yl)ethanol (1.80 g, 8.70 mmol in DCM (40 mL) was added TEA (2.63 g, 26.0 mmol) and TBSCl (1.96 g, 13.0 mmol) in portions at RT. The resulting solution was stirred for 16 h at RT. The resulting mixture was concentrated under vacuum. The residue was eluted from silica gel with EtOAc/PE (1:9). This resulted in 2 g (72%) of the title compound as yellow oil. MS-ESI: 322/324 (M+1).

### Step 5: 2-(4-(2-((Tert-butyldimethylsilyl)oxy)ethyl)thiazol-2-yl)propan-2-ol

To a stirred solution of 2-bromo-4-(2-((tert-butyldimethylsilyl)oxy)ethyl)thiazole (2.0 g, 6.23 mmol) in THF (50 mL) under nitrogen was added n-BuLi (2.5 M in hexane, 3.74 mL, 9.35 mmol) dropwise at −78° C. over 15 min. The resulting solution was stirred for 30 min at −78° C., then acetone (3.24 g, 55.9 mmol) was added dropwise at −78° C. The resulting mixture was quenched with water/ice (20 mL), and extracted with EtOAc (3×50 mL). The organic layers were combined, washed with brine (100 mL), dried over anhydrous Na₂SO₄, and concentrated under vacuum. The residue was eluted from silica gel with EtOAc/PE (1:2). This resulted in 1.65 g (88.0%) of the title compound as orange oil. MS-ESI: 302 (M+1).

Steps 6-9 used similar procedures for converting compound 420′ to Intermediate 96′ shown in Scheme 71 to afford Intermediate 104 from compound 468′. MS-ESI: 494 (M+1).

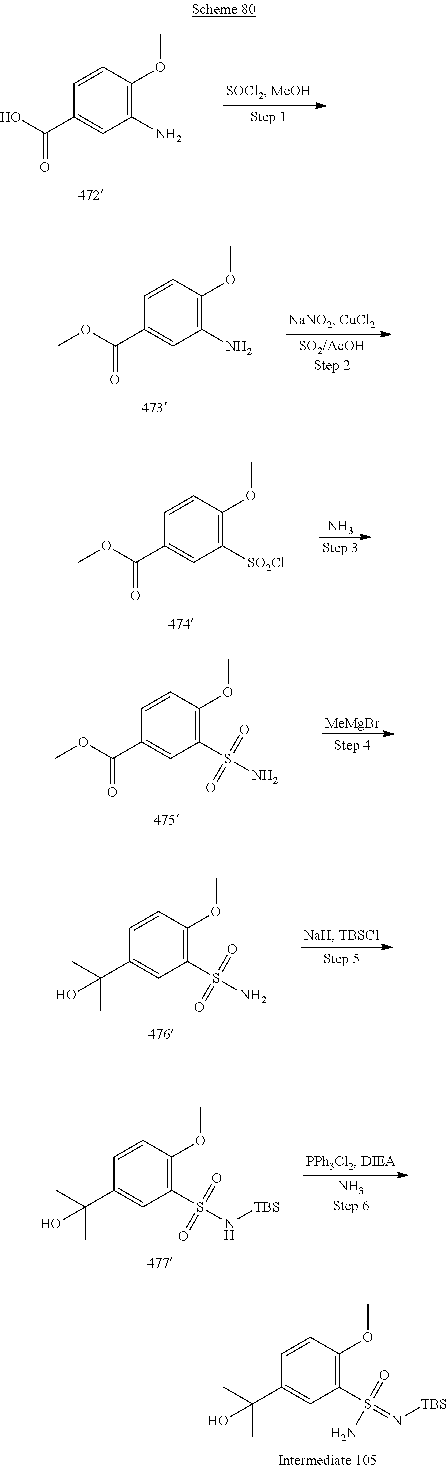

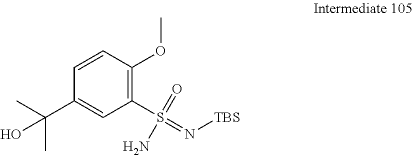

### N′-(tert-butyldimethylsilyl)-5-(2-hydroxypropan-2-yl)-2-methoxybenzenesulfonimidamide

### Step 1: Methyl 3-amino-4-methoxybenzoate

To a stirred solution of 3-amino-4-methoxybenzoic acid (5.00 g, 29.9 mmol) in MeOH (50 mL) under nitrogen was added SOCl₂ (35.6 g, 299 mmol) dropwise at RT. The resulting solution was stirred for 2 h at 60° C. The resulting mixture was concentrated and then diluted with 50 mL of water. The pH value of the mixture was adjusted to 7 with NaOH (1 M). The resulting solution was extracted with 3×100 mL of EtOAc. The combined organic layers were dried over anhydrous Na₂SO₄ and concentrated under vacuum. This resulted in 5 g (92.3%) of the title compound as a brown solid. MS-ESI: 182 (M+1).

### Step 2: Methyl 3-(chlorosulfonyl)-4-methoxybenzoate

To a stirred solution of methyl 3-amino-4-methoxybenzoate (5.0 g, 27.6 mmol) in aq. HCl (6 M, 100 mL) under nitrogen was added NaNO₂ (2.86 g, 41.4 mmol) in portions at 0° C. The resulting solution was stirred for 30 min at 0° C. This solution was assigned as A. SO₂ (g) was bubbled in AcOH (100 mL) for 10 min at 10° C., then to this solution was added CuCl₂ (0.74 g, 5.56 mmol) in portions at RT. This mixture was assigned as B. To the mixture B was added solution A dropwise below 10° C. The resulting solution was stirred for 2 h at RT. The resulting mixture was extracted with 3×200 mL of EtOAc. The organic layers were combined, washed with brine (200 mL), then dried over anhydrous Na₂SO₄, and concentrated under vacuum. This resulted in 1.8 g (25%) of the title compound as brown oil.

### Step 3: Methyl 4-methoxy-3-sulfamoylbenzoate

To a stirred solution of methyl 3-(chlorosulfonyl)-4-methoxybenzoate (1.8 g, 6.82 mmol) in DCM (20 mL) was bubbled NH₃ for 10 min at 0° C. The resulting solution was stirred for 2 h at RT. The resulging mixture was concentrated under vacuum. The residue was eluted from silica gel with EtOAc/PE (2:3). This resulted in 800 mg (47.9%) of the title compound as brown oil. MS-ESI: 244 (M−1).

### Step 4: 5-(2-Hydroxypropan-2-yl)-2-methoxybenzenesulfonamide

To a stirred solution of methyl 4-methoxy-3-sulfamoylbenzoate (800 mg, 3.27 mmol) in THF (5 mL) under nitrogen was added MeMgBr (3 M in ether, 3.3 mL, 9.9 mmol) dropwise at 0° C. The resulting solution was stirred at RT for 2 h. The reaction was then quenched with 10 mL of water and extracted with 3×30 mL of EtOAc. The organic layers were combined, washed with brine (20 mL), dried over anhydrous Na₂SO₄, and concentrated under vacuum. The residue was eluted from a silica gel column with EtOAc/PE (1:1). This resulted in 600 mg (74.9%) of the title compound as a dark yellow solid. MS-ESI: 244 (M−1).

Steps 5-6 used similar procedures for converting compound 417′ to Intermediate 95′ shown in Scheme 70 to afford Intermediate 105 from compound 476′. MS-ESI: 359 (M+1).

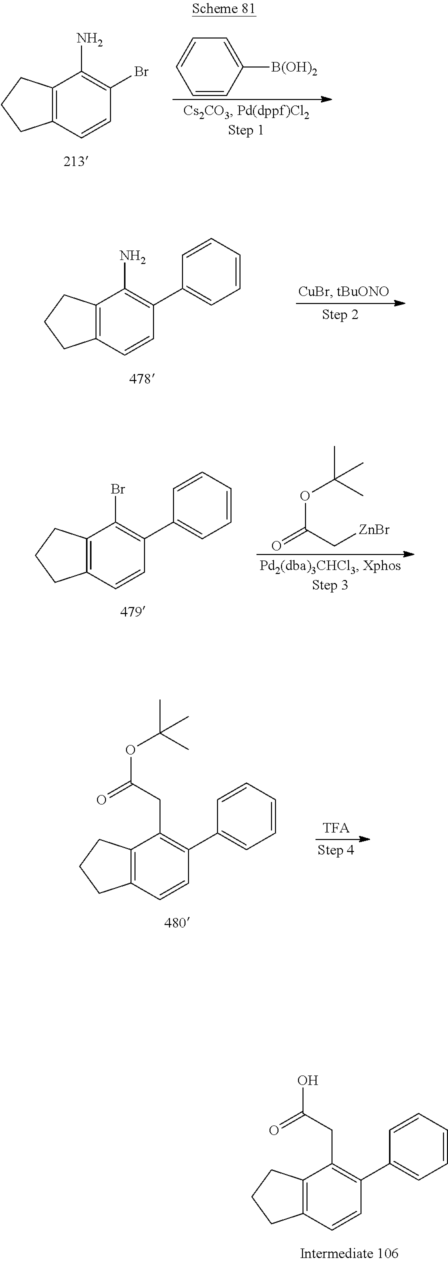

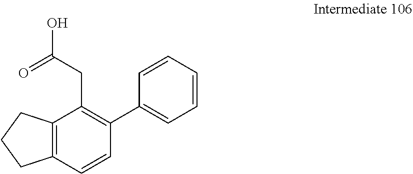 [SMILES: O=C(O)Cc1c(-c2ccccc2)ccc2c1CCC2]

### 2-(5-Phenyl-2,3-dihydro-1H-inden-4-yl)acetic Acid

Step 1 used similar procedures for converting compound 427′ to compound 428′ shown in Scheme 73 to afford compound 478′ from compound 213′. MS-ESI: 210 (M+1).

### Step 2: 4-Bromo-5-phenyl-2,3-dihydro-1H-indene

To a stirred solution of 5-phenyl-2,3-dihydro-1H-inden-4-amine (900 mg, 4.31 mmol) in ACN (20 mL) under nitrogen was added CuBr (1.23 g, 8.66 mmol) in portions and tert-butyl nitrite (0.89 g, 8.64 mmol) dropwise at RT. After stirring for 16 h at 70° C., the resulting mixture was concentrated under vacuum. The residue was purified by Prep-TLC with DCM/MeOH (10:1) to afford the title compound (400 mg, 34.0%) as a red solid. MS-ESI: 273/275 (M+1).

### Step 3: Tert-butyl 2-(5-phenyl-2,3-dihydro-1H-inden-4-yl)acetate

To a stirred solution of 4-bromo-5-phenyl-2,3-dihydro-1H-indene (270 mg, 0.993 mmol) in THF (10 mL) under nitrogen was added Xphos (57.2 mg, 0.099 mmol) and Pd₂(dba)₃CHCl₃ (102 mg, 0.099 mmol) at RT. After stirring for 10 min at RT under nitrogen, (2-(tert-butoxy)-2-oxoethyl)zinc(II) bromide (513 mg, 1.99 mmol) was added in portions at RT. After stirring for 12 h at 65° C., the resulting mixture was concentrated under vacuum. The residue was purified by Prep-TLC (PE/EtOAc=20:1) to afford the title compound (270 mg, 88.2%) as a red solid. ¹H NMR (300 MHz, CDCl₃) δ 7.46-7.25 (m, 4H), 7.21 (d, J=7.6 Hz, 1H), 7.14-7.01 (m, 2H), 3.49 (s, 2H), 3.01 (t, J=7.5 Hz, 2H), 2.92 (t, J=7.5 Hz, 2H), 2.25-2.06 (m, 2H), 1.43 (s, 9H).

### Step 4: 2-(5-Phenyl-2,3-dihydro-1H-inden-4-yl)acetic Acid

To a stirred solution of tert-butyl 2-(5-phenyl-2,3-dihydro-1H-inden-4-yl)acetate (100 mg, 0.325 mmol) in DCM (5 mL) was added TFA (2.62 g, 27.0 mmol) dropwise at RT. The resulting solution was stirred for 2 h at RT. The resulting mixture was concentrated and purified by Prep-TLC (PE/EtOAc=5:1). This resulted in 80 mg (97.6%) of the title compound as yellow oil. MS-ESI: 251 (M−1).

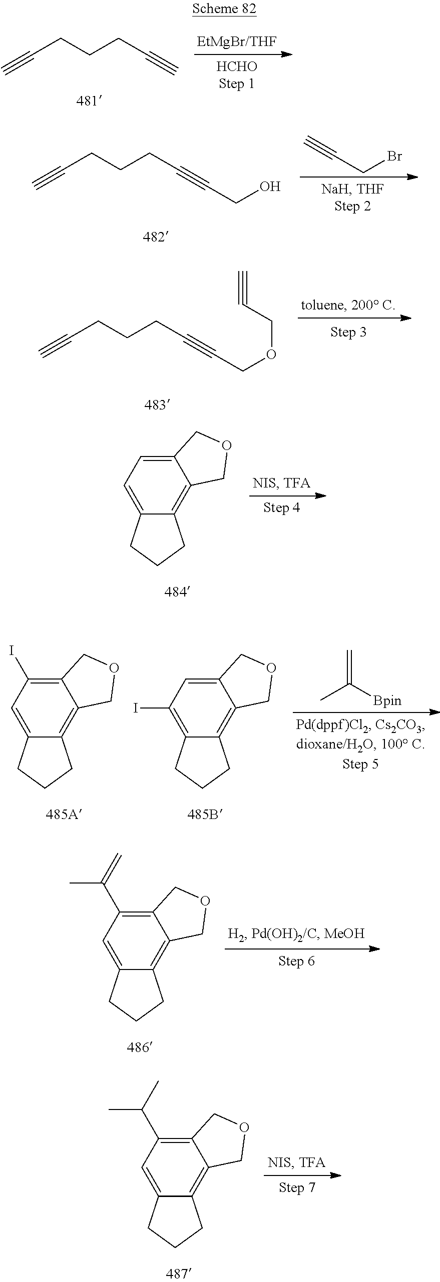

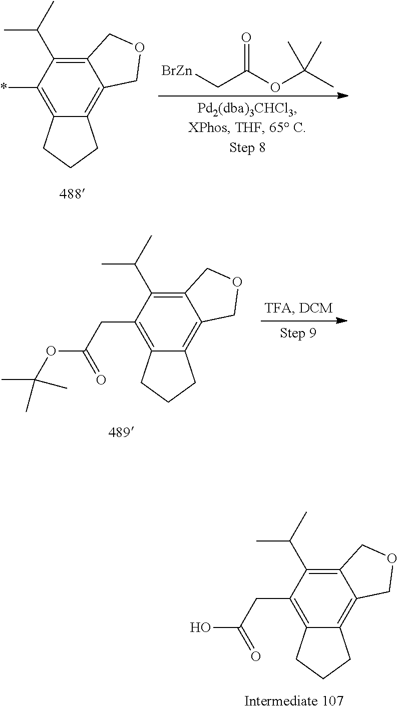

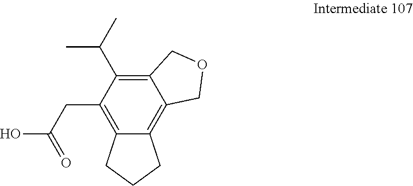 [SMILES: CC(C)c1c2c(c3c(c1CC(=O)O)CCC3)COC2]

### 2-(4-Isopropyl-3,6,7,8-tetrahydro-1H-indeno[4,5-c]furan-5-yl)acetic Acid

### Step 1: Octa-2,7-diyn-1-ol

To a stirred solution of hepta-1,6-diyne (20.0 g, 217 mmol) in THF (200 mL) under nitrogen was added EtMgBr (3 M in ether, 87 mL, 261 mmol) dropwise with stirring at 0° C. over 10 min. The resulting solution was stirred for 2 h at 0° C. Then HCHO (7.83 g, 261 mmol) was added in portions to the mixture at 0° C. The resulting mixture was stirred for an additional 20 h at 55° C. The reaction was then quenched with 20 mL of ice/water. The resulting mixture was diluted with 250 mL of sat. NH₄Cl (aq.) and 80 mL of 10% HCl (aq.). The resulting solution was extracted with 200 mL of EtOAc, the organic layer was collected, washed with 200 mL of sat. NaHCO₃(aq.), dried over anhydrous sodium sulfate, then concentrated under vacuum. The residue was eluted from a silica gel column with EtOAc/PE (1:3). This resulted in 22.0 g (83%) of the title compound as yellow oil. ¹H NMR (400 MHz, CDCl₃) δ 4.27 (t, J=2.2 Hz, 2H), 2.43-2.28 (m, 4H), 1.99 (t, J=2.6 Hz, 1H), 1.79 (br s, 1H), 1.79-1.69 (m, 2H).

### Step 2: 8-(Prop-2-yn-1-yloxy)octa-1,6-diyne

To a stirred mixture of NaH (60% wt, 7.2 g, 180 mmol) in THF (200 mL) under nitrogen was added octa-2,7-diyn-1-ol (22.0 g, 180 mmol) in THF (10 mL) dropwise with stirring at 0° C. The resulting solution was stirred for 2 h at 0° C. 3-bromoprop-1-yne (20.1 g, 169 mmol) was added to the mixture at 0° C. The resulting solution was stirred for an additional 30 h at RT. The reaction was quenched with 200 mL of ice/water and extracted with 2×100 mL of EtOAc. The organic layers were combined, dried over anhydrous sodium sulfate, and concentrated under vacuum. The residue was eluted from a silica gel column with EtOAc/PE (1:4). This resulted in 16.5 g (57%) of the title compound as a yellow oil. ¹H NMR (400 MHz, CDCl₃) 4.40-4.15 (m, 4H), 2.46 (t, J=2.4 Hz, 1H), 2.39 (tt, J=7.0, 2.2 Hz, 2H), 2.34 (td, J=7.0, 2.6 Hz, 2H), 1.99 (t, J=2.8 Hz, 1H), 1.89-1.70 (m, 2H).

### Step 3: 3,6,7,8-Tetrahydro-1H-indeno[4,5-c]furan

The solution of 8-(prop-2-yn-1-yloxy)octa-1,6-diyne (2.1 g, 13.1 mmol) in toluene (20 mL) in a 50-mL steel sealed tube was stirred for 16 h at 200° C. The reaction was cooled to RT and concentrated under vacuum. The residue was eluted from a silica gel column with EtOAc/PE (1:3). This resulted in 2.0 g (95%) of the title compound as yellow oil. ¹H NMR (400 MHz, CDCl₃) δ 7.18 (d, J=7.6 Hz, 1H), 7.05 (d, J=7.6 Hz, 1H), 5.14 (s, 2H), 5.09 (s, 2H), 2.96 (t, J=7.4 Hz, 2H), 2.83 (t, J=7.6 Hz, 2H), 2.30-2.05 (m, 2H).

### Step 4: 4-Iodo-3,6,7,8-tetrahydro-1H-indeno[4,5-c]furan and 5-iodo-3,6,7,8-tetrahydro-1H-indeno[4,5-c]furan

To a stirred solution of 3,6,7,8-tetrahydro-1H-indeno[4,5-c]furan (2.0 g, 12.5 mmol) in TFA (40 mL) was added NIS (3.37 g, 15.0 mmol) in portions at 0° C. The resulting mixture was stirred for 16 h at RT under nitrogen. The resulting mixture was concentrated under vacuum. The resulting mixture was dissolved in DCM (50 mL) and washed with sat. aq. Na₂CO₃ (20 mL) at RT. The organic phase was collected and the aqueous layer was extracted with DCM (3×10 mL). The combined organic layers were dried over anhydrous sodium sulfate and concentrated under vacuum. The residue was eluted from silica gel column with PE/EtOAc (9:1) to afford 4-iodo-1H,3H,6H,7H,8H-indeno[4,5-c]furan (485A′, 882 mg, 24.7%) and 5-iodo-1H,3H,6H,7H,8H-indeno[4,5-c]furan (485B′, 1.31 g, 36.7%) both as a light yellow solid. Compound 485A′ ¹H NMR (400 MHz, CDCl₃) δ 7.50 (s, 1H), 5.20 (s, 2H), 5.02 (s, 2H), 2.93 (t, J=7.5 Hz, 2H), 2.75 (t, J=7.5 Hz, 2H), 2.25-2.00 (m, 2H). NOESY: Ar—H at 7.50 (s, 1H) has correlation with cyclopenta's CH₂ at 2.93 (t, J=7.5 Hz, 2H).

Compound 485B′ ¹H NMR (400 MHz, CDCl₃) δ 7.44 (s, 1H), 5.08 (s, 2H), 5.00 (s, 2H), 3.05-2.80 (m, 4H), 2.50-2.20 (m, 2H). NOESY: Ar—H at 7.44 (s, 1H) has correlation with O—CH₂ at 5.08 (s, 2H) and 5.00 (s, 2H).

### Step 5: 4-(Prop-1-en-2-yl)-3,6,7,8-tetrahydro-1H-indeno[4,5-c]furan

To a stirred mixture of 4-iodo-3,6,7,8-tetrahydro-1H-indeno[4,5-c]furan (600 mg, 2.1 mmol) in dioxane (30 mL) and H₂O (3 mL) under nitrogen was added 4,4,5,5-tetramethyl-2-(prop-1-en-2-yl)-1,3,2-dioxaborolane (1.76 g, 10.5 mmol), Pd(dppf)Cl₂ (155 mg, 0.21 mmol) and Cs₂CO₃ (2.05 g, 6.3 mmol) in portions at RT. The resulting mixture was stirred for 16 h at 100° C. The reaction was diluted with sat. aq. NaCl (90 mL) at RT. The aqueous layer was extracted with EtOAc (3×90 mL). The combined organic layers were dried over anhydrous sodium sulfate and concentrated under vacuum. The residue was eluted from silica gel column with PE/EtOAc (9:1) to afford the title compound (267 mg, 63.6%) as a yellow oil. ¹H NMR (400 MHz, CDCl₃) δ 7.18 (s, 1H), 5.224 (s, 1H), 5.218 (s, 2H), 5.11 (s, 2H), 5.00 (s, 1H), 2.98 (t, J=7.4 Hz, 2H), 2.84 (t, J=7.4 Hz, 2H), 2.24-2.13 (m, 2H), 2.15 (s, 3H).

### Step 6: 4-Isopropyl-3,6,7,8-tetrahydro-1H-indeno[4,5-c]furan

To a stirred solution of 4-(prop-1-en-2-yl)-3,6,7,8-tetrahydro-1H-indeno[4,5-c]furan (700 mg, 3.50 mmol) in MeOH (15 mL) under nitrogen was added Pd(OH)₂/C (20% wt., 140 mg) in portions at RT. The flask was evacuated and refilled three times with hydrogen. The resulting mixture was stirred for 16 h at RT under hydrogen with a balloon. The resulting mixture was filtered and the filtrate was concentrated under vacuum to afford the title compound (650 mg, 91.9%) as a light yellow solid. GCMS-ES: 202 (M).

### Step 7: 5-Iodo-4-isopropyl-3,6,7,8-tetrahydro-1H-indeno[4,5-c]furan

To a stirred mixture of 4-isopropyl-3,6,7,8-tetrahydro-1H-indeno[4,5-c]furan (400 mg, 1.98 mmol) in TFA (8 mL) under nitrogen was added NIS (890 mg, 3.96 mmol) in portions at 0° C. The resulting mixture was stirred for 16 h at RT. The resulting mixture was concentrated under vacuum. The residue was dissolved in EtOAc (50 mL) and washed with sat. aq. Na₂CO₃ (20 mL). The organic layer was collected and the aqueous layer was extracted with EtOAc (3×20 mL). The combined organic layer was dried over anhydrous sodium sulfate and concentrated under vacuum. The residue was purified by Prep-TLC (PE/EtOAc 9:1) to afford the title compound (190 mg, 29.3%) as an orange solid. GCMS-ES: 328 (M).

### Step 8: Tert-butyl 2-(4-isopropyl-3,6,7,8-tetrahydro-1H-indeno[4,5-c]furan-5-yl)acetate

To a stirred solution of 5-iodo-4-isopropyl-3,6,7,8-tetrahydro-1H-indeno[4,5-c]furan (150 mg, 0.457 mmol) in THF (15 mL) under nitrogen was added Pd₂(dba)₃CHCl₃ (47 mg, 0.046 mmol), XPhos (22 mg, 0.046 mmol) and (2-(tert-butoxy)-2-oxoethyl)zinc(II) bromide (1.19 g, 4.57 mmol) in portions at RT. The resulting mixture was stirred for 2 h at 65° C. The reaction was quenched with sat. aq. NH₄Cl (15 mL). The resulting mixture was filtered, and the filter cake was washed with EtOAc (3×15 mL). The aqueous layer was extracted with EtOAc (3×20 mL). The combined organic layer was dried over anhydrous sodium sulfate and concentrated under vacuum. The residue was purified by Prep-TLC (PE/EtOAc=7:1) to afford the title compound (47 mg, 32.5%) as a brown solid. ¹H NMR (400 MHz, CDCl₃) δ 5.25 (s, 2H), 5.00 (s, 2H), 3.66 (s, 2H), 3.28 (hep, J=7.1 Hz, 1H), 2.93 (t, J=7.6 Hz, 2H), 2.82 (t, J=7.5 Hz, 2H), 2.40-5.10 (m, 2H), 1.36-1.21 (m, 15H).

### Step 9: 2-(4-Isopropyl-3,6,7,8-tetrahydro-1H-indeno[4,5-c]furan-5-yl)acetic Acid

To a stirred solution of tert-butyl 2-(4-isopropyl-3,6,7,8-tetrahydro-1H-indeno[4,5-c]furan-5-yl)acetate (47 mg, 0.149 mmol) in DCM (10 mL) was added TFA (2.5 mL) dropwise at 0° C. The resulting mixture was stirred for 2 h at RT. The resulting mixture was concentrated under vacuum. This resulted in the title compound (55 mg crude) as a yellow solid which was used for next step without further purification. MS-ESI: 259 (M−1).

Schemes of Sulfonimidamide and phenylacetic acid Intermediates: Schemes below illustrate the preparation of sulfonimidamide and phenylacetic acid intermediates.

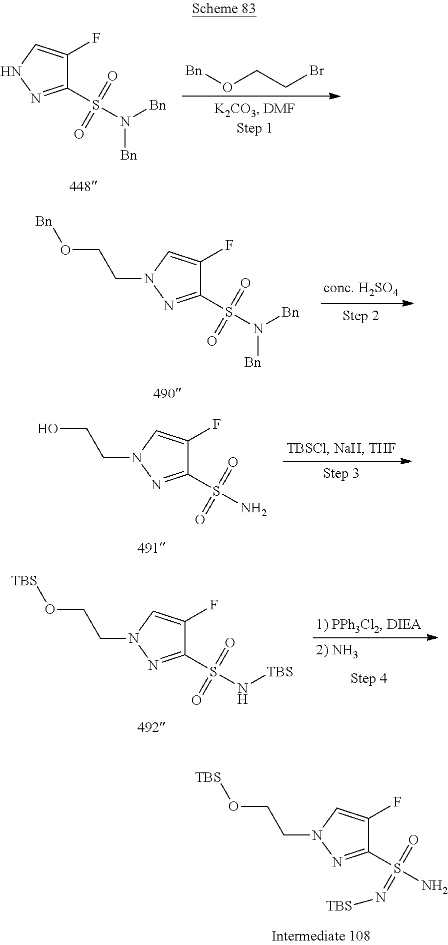

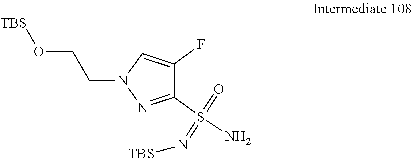

### N′-(tert-butyldimethylsilyl)-1-(2-((tert-butyldimethylsilyl)oxy)ethyl)-4-fluoro-1H-pyrazole-3-sulfonimidamide

### Step 1: N,N-dibenzyl-1-(2-(benzyloxy)ethyl)-4-fluoro-1H-pyrazole-3-sulfonamide

To a stirred solution of N,N-dibenzyl-4-fluoro-1H-pyrazole-3-sulfonamide (2.50 g, 7.25 mmol) in DMF (10 mL) was added ((2-bromoethoxy)methyl)benzene (1.87 g, 8.70 mmol) and K₂CO₃ (1.50 g, 10.88 mmol). The resulting solution was stirred for 16 h at 65° C. The resulting solution was filtered; the filter cake was washed with EtOAc (3×10 mL). The wash and filtrate were combined, diluted with water (100 mL) and then extracted with EtOAc (3×50 mL). The combined organic layers were dried over Na₂SO₄ and concentrated under vacuum. The residue was purified by Prep-TLC (EA:PE=1:4). This resulted in 3.0 g (86.4%) of the title compound as light yellow oil. MS-ESI: 480 (M+1).

### Step 2: 4-Fluoro-1-(2-hydroxyethyl)-1H-pyrazole-3-sulfonamide

To a stirred solution of N,N-dibenzyl-1-(2-(benzyloxy)ethyl)-4-fluoro-1H-pyrazole-3-sulfonamide (3.0 g, 6.25 mmol) in DCM (2 mL) was added conc. H₂SO₄ (2 mL) dropwise at 0° C. The reaction was poured into water/ice (15 mL) slowly. The pH of this mixture was adjusted to 6 with sat. NaOH (aq). The resulting solution was filtered; the filter cake was washed with MeOH (5×10 mL). The wash and filtrate were combined and concentrated under vacuum. The residue was eluted from a silica gel column with MeOH/DCM (1:10). This resulted in 1.1 g (84.2%) of the title compound as a yellow semi-solid. MS-ESI: 208 (M−1).

### Step 3: N-(tert-butyldimethylsilyl)-1-(2-((tert-butyldimethylsilyl)oxy)ethyl)-4-fluoro-1H-pyrazole-3-sulfonamide

To a stirred solution of 4-fluoro-1-(2-hydroxyethyl)-1H-pyrazole-3-sulfonamide (1.10 g, 5.26 mmol) in THF (80 mL) under nitrogen was added NaH (60% wt., 2.10 g, 52.6 mmol) in portions at 0° C. The resulting solution was stirred for 0.5 h at RT. To this was added TBSCl (7.94 g, 52.6 mmol) in portions at 0° C. The resulting solution was stirred for 16 h at 60° C. The reaction was then quenched with 200 mL of water/ice, extracted with 3×200 mL of EtOAc. The organic layers were combined and concentrated under vacuum. The residue was eluted from a silica gel column with EtOAc/PE (1:4). This resulted in 1.6 g (69.6%) of the title compound as a white solid. MS-ESI: 438 (M+1)

### Step 4: N′-(tert-butyldimethylsilyl)-1-(2-((tert-butyldimethylsilyl)oxy)ethyl)-4-fluoro-1H-pyrazole-3-sulfonimidamide

To a stirred solution of PPh₃Cl₂ (3.61 g, 10.8 mmol) in CHCl₃ (20 mL) under nitrogen was added DIEA (2.24 g, 17.4 mmol) dropwise at 0° C. The resulting solution was stirred for 20 min at 0° C. To the above solution was added N-(tert-butyldimethylsilyl)-1-(2-((tert-butyldimethylsilyl)oxy)ethyl)-4-fluoro-1H-pyrazole-3-sulfonamide (950 mg, 2.17 mmol) in CHCl₃ (5 mL) dropwise at 0° C. The resulting solution was stirred for 1 h at 0° C. To the above solution was introduced NH₃ bubbled at 0° C. for 5 min. The resulting solution was stirred for 16 h at RT. The solids were filtered out. The filtrate was concentrated under vacuum. The residue was eluted from a silica gel column with EtOAc/PE (1:2). This resulted in 620 mg (65.4%) of the title compound as a light yellow solid. MS-ESI: 437 (M+1).

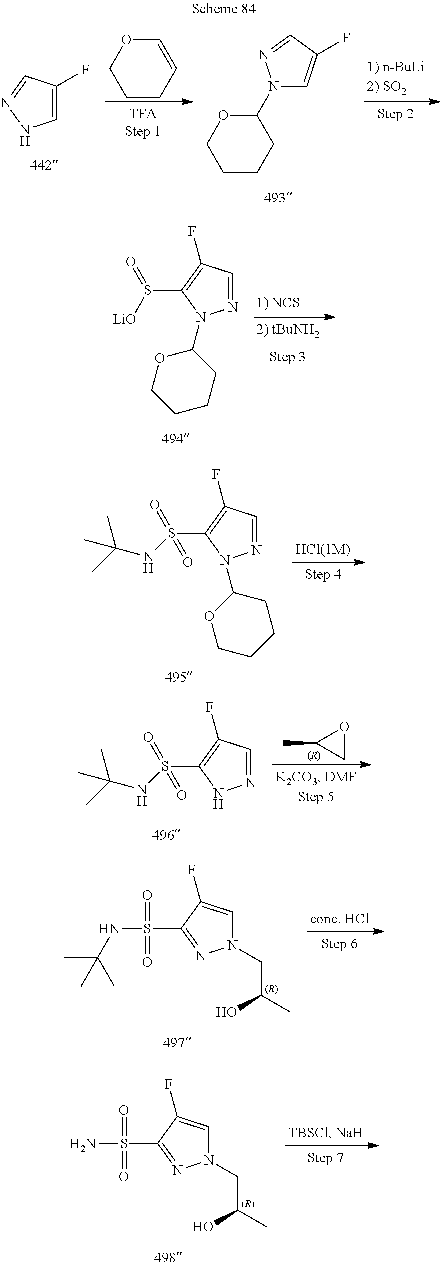

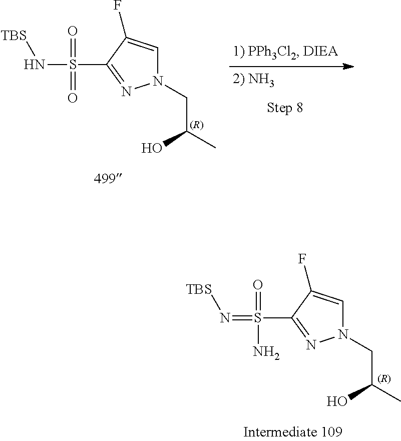

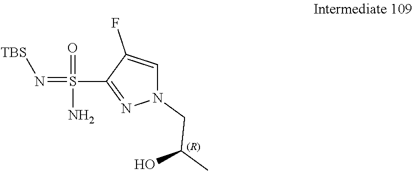

### N′-(tert-butyldimethylsilyl)-4-fluoro-1-((R)-2-hydroxypropyl)-1H-pyrazole-3-sulfonimidamide

### Step 1: 4-Fluoro-1-(tetrahydro-2H-pyran-2-yl)-1H-pyrazole

To a stirred solution of 4-fluoro-1H-pyrazole (5.0 g, 58.1 mmol) in 3,4-dihydro-2H-pyran (9.77 g, 116 mmol) under nitrogen was added the catalytic amount of TFA (260 mg, 2.32 mmol). The resulting solution was stirred for 16 h at 100° C. The reaction was then quenched with 200 mg of NaH (60% wt.) at 0° C. The resulting solution was diluted with water (100 mL). The resulting solution was extracted with EtOAc (3×100 mL). The organic layers were combined and dried over anhydrous Na₂SO₄. After filtration, the filtrate was concentrated under vacuum. This resulted in 12.0 g (crude) of the title compound as a light brown oil. MS-ESI: 171 (M+1).

### Step 2: Lithium 4-fluoro-1-(tetrahydro-2H-pyran-2-yl)-1H-pyrazole-5-sulfinate

To a stirred solution of 4-fluoro-1-(oxan-2-yl)pyrazole (12.0 g, crude from last step) in THF (250 mL) was added n-BuLi (2.5 M in hexane, 20.0 mL, 50 mmol) dropwise at −78° C. The resulting solution was stirred for 40 min at −78° C. To the above solution was introduced SO₂(g) bubbled at −78° C. for 5 min. Then, the temperature was warmed to RT. The resulting solution was stirred for an additional 1 h at RT. The resulting solution was concentrated under vacuum. This resulted in 28.0 g (crude) of the title compound as a light yellow semi-solid. MS-ESI: 233 (M−1).

### Step 3: N-(tert-butyl)-4-fluoro-1-(tetrahydro-2H-pyran-2-yl)-1H-pyrazole-5-sulfonamide

To a stirred solution of lithium 4-fluoro-1-(tetrahydro-2H-pyran-2-yl)-1H-pyrazole-5-sulfinate (28.0 g, crude from last step) in ACN (200 mL) was added NCS (19.9 g, 149 mmol) in ACN (50 mL) dropwise at 0° C. The resulting solution was stirred for 1 h at RT. To the reaction mixture above was added tBuNH₂ (49.8 g, 683 mmol) dropwise at 0° C. The resulting solution was stirred for an additional 30 min at RT, concentrated under vacuum, and diluted with 200 mL of water. The resulting solution was extracted with 3×150 mL of EtOAc, and the organic layers were combined concentrated under reduced pressure. The residue was purified by Prep-TLC with EtOAc/PE (1:4). This resulted in 13.3 g (75.0% over three steps) of the title compound as a yellow solid. MS-ESI: 304 (M−1).

### Step 4: N-(tert-butyl)-4-fluoro-1H-pyrazole-5-sulfonamide

To a stirred solution of N-(tert-butyl)-4-fluoro-1-(tetrahydro-2H-pyran-2-yl)-1H-pyrazole-5-sulfonamide (13.3 g, 43.6 mmol) in MeOH (220 mL) was added HCl (conc., 20 mL) dropwise at 0° C. The resulting solution was stirred for 30 min at RT. The resulting solution was concentrated under vacuum. The residue was eluted from a silica gel column with EtOAc/PE (1:1). This resulted in 7.50 g (77.8%) of the title compound as a yellow solid. MS-ESI: 222 (M+1).

### Step 5: (R)—N-(tert-butyl)-4-fluoro-1-(2-hydroxypropyl)-1H-pyrazole-3-sulfonamide

To a stirred solution of N-(tert-butyl)-4-fluoro-1H-pyrazole-5-sulfonamide (1.0 g, 4.52 mmol) in DMF (20 mL) under nitrogen was added K₂CO₃ (1.87 g, 13.56 mmol) and (R)-2-methyloxirane (524 mg, 9.04 mmol). The resulting solution was stirred for 16 h at 100° C. The reaction was quenched with 25 mL of water, and extracted with 3×30 mL of EtOAc. The organic layers were combined, dried over anhydrous Na₂SO₄ and concentrated under vacuum. The residue was eluted from a silica gel column with EtOAc/PE (1:3). This resulted in 820 mg (65.0%) of the title compound as a yellow solid. MS-ESI 280 (M+1).

### Step 6: (R)-4-fluoro-1-(2-hydroxypropyl)-1H-pyrazole-3-sulfonamide

To stirred concentrated HCl (10 mL) was added (R)—N-(tert-butyl)-4-fluoro-1-(2-hydroxypropyl)-1H-pyrazole-3-sulfonamide (820 mg, 2.94 mmol) in portions at 0° C. The resulting solution was stirred for 2 h at RT and concentrated under vacuum. The residue was eluted from a silica gel column with EtOAc (100%). This resulted in 600 mg (91.5%) of the title compound as yellow oil. MS-ESI: 224 (M+1). ¹H NMR (400 MHz, DMSO-d₆) δ 7.98 (d, J=4.7 Hz, 1H), 7.68 (s, 2H), 5.03 (d, J=4.3 Hz, 1H), 4.09-3.90 (m, 3H), 1.07 (d, J=5.8 Hz, 3H). NOESY: Ar—H at 7.98 (d, J=4.7 Hz, 1H) has correlation with CH₂ at 4.09-3.90 (m, 3H).

Steps 7-8 used similar procedures for converting compound 491″ to Intermediate 108 shown in Scheme 83 to afford Intermediate 109 from compound 498″. MS-ESI: 337 (M+1). ¹H NMR (400 MHz, DMSO-d₆) δ 7.87 (d, J=4.8 Hz, 1H), 6.86 (s, 2H), 5.00 (br s, 1H), 4.07-3.89 (m, 3H), 1.10-1.02 (m, 3H), 0.86 (s, 9H), 0.04 (s, 6H).

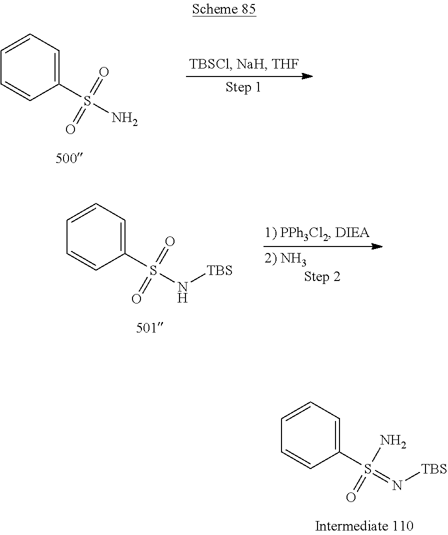

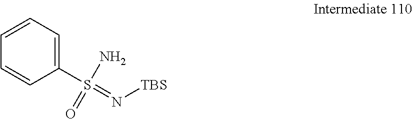

### N′-(tert-butyldimethylsilyl)benzenesulfonimidamide

Steps 1-2 used similar procedures for converting compound 491″ to Intermediate 108 shown in Scheme 83 to afford Intermediate 110 from compound 500″. MS-ESI. 271 (M+1).

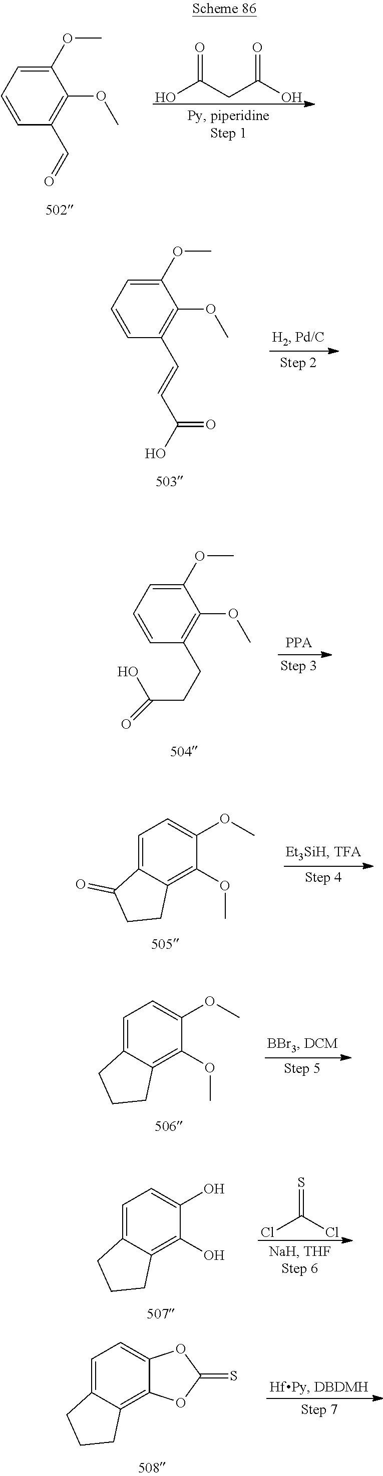

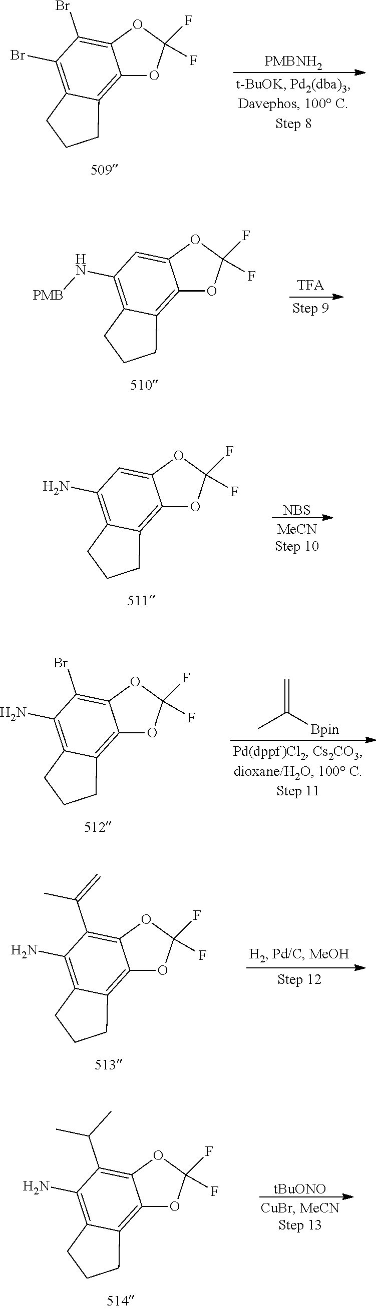

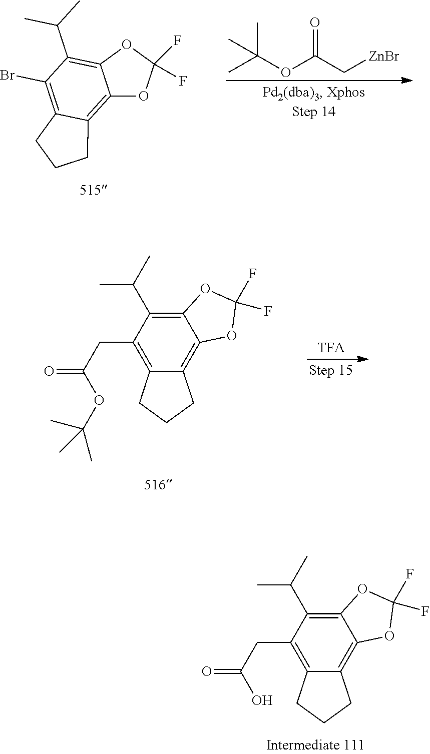

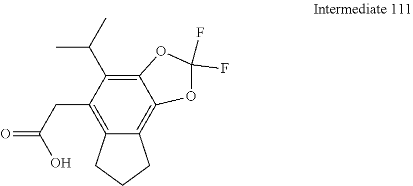 [SMILES: CC(C)c1c(CC(=O)O)c2c(c3c1OC(F)(F)O3)CCC2]

### 2-(2,2-Difluoro-4-isopropyl-7,8-dihydro-6H-indeno[4,5-d][1,3]dioxol-5-yl)acetic Acid

### Step 1: (E)-3-(2,3-dimethoxyphenyl)acrylic Acid

To a solution of 2,3-dimethoxybenzaldehyde (50.0 g, 301 mmol) in pyridine (120 mL) under nitrogen was added malonic acid (61.0 g, 586 mmol) in portions at RT. The resulting solution was heated to 50° C. with stirring until the malonic acid dissolved. Then, piperidine (5 mL, 55 mmol) was added into the above reaction mixture. The resulting solution was stirred for 1 h at 80° C., and then heated reflux 16 h. The reaction was then cooled to 0° C. and quenched with 300 mL of water. The pH of the solution was acidified to 1 with conc. HCl. The precipitated product was collected by filtration and washed with water (3×100 mL). The filter cake was then dissolved in 2 M NaOH (aq.). The resulting solution was filtered. Filtrate was diluted with water (100 mL) and acidified with conc. HCl to adjust the pH=1. The product was collected by filtration and washed with water (3×100 mL). The white filter cake was dissolved in EtOAc (300 mL) to give a solution that was washed with brine (3×100 mL), dried over anhydrous Na₂SO₄, and evaporated. This resulted in 62.0 g (99.0%) of the title compound as a white solid. MS-ESI: 207 (M−1). ¹H NMR (400 MHz, DMSO-d₆) δ 12.43 (br s, 1H), 7.79 (d, J=16.4 Hz, 1H), 7.39-7.25 (m, 1H), 7.19-7.02 (m, 2H), 6.51 (d, J=16.0 Hz, 1H), 3.83 (s, 3H), 3.76 (s, 3H).

### Step 2: 3-(2,3-Dimethoxyphenyl)propanoic Acid

To a stirred solution of (E)-3-(2,3-dimethoxyphenyl)acrylic acid (62.0 g, 298 mmol) in EtOH (200 mL) and EtOAc (200 mL) under nitrogen was added 5 drops of acetic acid and Pd/C (10% wt., 6.20 g). The flask was evacuated and refilled three times with hydrogen. The resulting solution was stirred for 16 h at RT under hydrogen with a balloon. The solids were filtered out. The filtrate was concentrated under vacuum. This resulted in 60.9 g (97.3%) of the title compound as a pink solid. MS-ESI: 209 (M−1). ¹H NMR (400 MHz, DMSO-d₆) δ 12.11 (s, 1H), 6.97 (t, J=7.8 Hz, 1H), 6.90 (dd, J=8.0, 1.2 Hz, 1H), 6.78 (dd, J=7.6, 1.2 Hz, 1H), 3.79 (s, 3H), 3.73 (s, 3H), 2.80 (t, J=7.8 Hz, 2H), 2.47 (t, J=7.8 Hz, 2H).

### Step 3: 4,5-Dimethoxy-2,3-dihydro-1H-inden-1-one

To polyphosphoric acid (300 g) was added 3-(2,3-dimethoxyphenyl)propanoic acid (50.0 g, 238 mmol) in portions at 70° C. with stirring. The resulting solution was stirred for 45 min at 70° C. Then, a further aliquot of polyphosphoric acid (100 g) was added and the mixture was stirred at 70° C. for a further 55 min. The reaction was then cooled to 0° C. and quenched with water/ice (500 mL). The mixture was extracted with 4×300 mL of EtOAc. The combined organic layers were washed with 2×300 mL of sat. NaHCO₃(aq.) followed by 2×200 mL of brine. The organic layer was dried over anhydrous Na₂SO₄ and concentrated under vacuum. This resulted in 42.9 g (93.9%) of the title compound as a light brown solid. ¹H NMR (400 MHz, DMSO-d₆) δ 7.40 (d, J=8.4 Hz, 1H), 7.15 (d, J=8.5 Hz, 1H), 3.91 (s, 3H), 3.82 (s, 3H), 3.07-2.99 (m, 2H), 2.62-2.54 (m, 2H).

### Step 4: 4,5-Dimethoxy-2,3-dihydro-1H-indene

To a solution of 4,5-dimethoxy-2,3-dihydro-1H-inden-1-one (40.0 g, 208 mmol) in TFA (60 mL) was added Et₃SiH (120 mL) dropwise at RT. The resulting solution was stirred for 16 h at RT and concentrated under vacuum. The residue was eluted from a silica gel column with EtOAc/PE (1:10). This resulted in 24.7 g (66.6%) of the title compound as colorless oil. ¹H NMR (400 MHz, DMSO-d₆) δ 6.88 (d, J=8.1 Hz, 1H), 6.79 (d, J=8.1 Hz, 1H), 3.74 (s, 3H), 3.71 (s, 3H), 2.83 (t, J=7.4 Hz, 2H), 2.79 (t, J=7.4 Hz, 2H), 2.00 (quint, J=7.4 Hz, 2H).

### Step 5: 2,3-Dihydro-1H-indene-4,5-diol

To a stirred solution of 4,5-dimethoxy-2,3-dihydro-1H-indene (24.0 g, 135 mmol) in DCM (90 mL) was added BBr₃ (1 M in DCM, 200 mL) dropwise at 0° C. The resulting solution was stirred for 16 h at RT. The reaction was then quenched with 200 mL of water/ice and extracted with 3×200 mL of DCM. The organic layers were combined and dried over anhydrous Na₂SO₄ and concentrated under vacuum. The residue was eluted from a silica gel column with EtOAc/PE (1:6). This resulted in 12.2 g (60.1%) of the title compound as a yellow solid. MS-ESI: 149 (M−1). ¹H NMR (400 MHz, DMSO-d₆) δ 8.66 (s, 1H), 8.20 (s, 1H), 6.54 (d, J=7.8 Hz, 1H), 6.47 (d, J=7.8 Hz, 1H), 2.72 (t, J=7.4 Hz, 4H), 1.95 (quint, J=7.4 Hz, 2H).

### Step 6: 7,8-Dihydro-6H-indeno[4,5-d][1,3]dioxole-2-thione

To a stirred solution of 2,3-dihydro-1H-indene-4,5-diol (8.93 g, 59.5 mmol) in THF (200 mL) under nitrogen was added NaH (60% wt., 4.76 g, 119 mmol) in portions at 0° C. The resulting solution was stirred for 20 min at 0° C. This was followed by the addition of a solution of thiophosgene (10.3 g, 89.3 mmol) in THF (50 mL) dropwise with stirring at 0° C. The resulting solution was stirred for 2 h at RT. The reaction was then quenched with 500 mL of water/ice, extracted with 3×400 mL of EtOAc. The organic layers were combined and dried over anhydrous Na₂SO₄. The residue was eluted from a silica gel column with EtOAc/PE (1:3). This resulted in 8.0 g (70.0%) of the title compound as a yellow solid. ¹H NMR (400 MHz, DMSO-d₆) δ 7.40 (d, J=8.2 Hz, 1H), 7.28 (d, J=8.2 Hz, 1H), 3.04 (t, J=7.5 Hz, 2H), 2.97 (t, J=7.5 Hz, 2H), 2.16 (quint, J=7.5 Hz, 2H).

### Step 7: 4,5-Dibromo-2,2-difluoro-7,8-dihydro-6H-indeno[4,5-d][1,3]dioxole

To a stirred solution of DBDMH (35.7 g, 125 mmol) in DCM (200 mL) under nitrogen was added HF-Pyridine (70% wt., 48 mL) dropwise with stirring at −50° C. To the above solution was added 6H,7H,8H-indeno[4,5-d][1,3]dioxole-2-thione (8.0 g, 41.7 mmol) in DCM (80 mL) dropwise with stirring at −50° C. The resulting solution was stirred for 1 h at −50° C. and then warmed to RT. The resulting solution was stirred for an additional 1 h at RT. The reaction was then quenched with 250 mL of water/ice. The resulting solution was extracted with 2×400 mL of EtOAc, the organic layers were combined and dried over anhydrous Na₂SO₄ and concentrated under vacuum. The residue was eluted from a silica gel column with EtOAc/PE (1/100). This resulted in 7.0 g (47.1%) of the title compound as a white solid. MS-ESI: 355/357/359 (M+1).

### Step 8: 2,2-Difluoro-N-(4-methoxybenzyl)-7,8-dihydro-6H-indeno[4,5-d][1,3]dioxol-5-amine

To a stirred solution of 4,5-dibromo-2,2-difluoro-6H,7H,8H-indeno[4,5-d][1,3]dioxole (6.50 g, 18.3 mmol) in dioxane (80 mL) under nitrogen was added t-BuOK (6.15 g, 54.9 mmol), Pd₂(dba)₃ (1.67 g, 1.83 mmol), DavePhos (0.72 g, 1.83 mmol) and 4-methoxybenzylamine (10.0 g, 73.2 mmol). The resulting solution was stirred for 3 h at 100° C. The resulting solution was concentrated. The residue was eluted from a silica gel column with PE. This resulted in 2.49 g (40.9%) of the title compound as a dark orange solid. MS-ESI: 334 (M+1). ¹H NMR (400 MHz, CD₃OD) δ 7.31-7.23 (m, 2H), 6.92-6.84 (m, 2H), 6.18 (s, 1H), 4.27 (s, 2H), 3.77 (s, 3H), 2.90 (t, J=7.4 Hz, 2H), 2.74 (t, J=7.4 Hz, 2H), 2.17 (quint, J=7.4 Hz, 2H). NOESY: Ar—H at 6.18 (s, 1H) has correlation with benzyl's CH₂ at 4.27 (s, 2H) but no correlation with cyclopenta's CH₂ at 2.90 (t, J=7.4 Hz, 2H) or 2.74 (t, J=7.4 Hz, 2H).

### Step 9: 2,2-Difluoro-7,8-dihydro-6H-indeno[4,5-d][1,3]dioxol-5-amine

2,2-Difluoro-N-(4-methoxybenzyl)-7,8-dihydro-6H-indeno[4,5-d][1,3]dioxol-5-amine (2.49 g, 7.48 mmol) was dissolved in TFA (80 mL). The resulting solution was stirred for 2 h at 80° C. The resulting solution was concentrated. The residue was eluted from a silica gel column with EtOAc/PE (1:4). This resulted in 1.5 g (94.2%) of the title compound as a dark yellow solid. MS-ESI: 214 (M+1).

### Step 10: 4-Bromo-2,2-difluoro-7,8-dihydro-6H-indeno[4,5-d][1,3]dioxol-5-amine

To a stirred solution of 2,2-difluoro-7,8-dihydro-6H-indeno[4,5-d][1,3]dioxol-5-amine (1.50 g, 7.04 mmol) in ACN (50 mL) under nitrogen was added NBS (1.025 g, 7.04 mmol) in portions at RT. The resulting solution was stirred for 1 h at RT and then concentrated under vacuum to removed ACN. The residue was dissolved in EtOAc (50 mL) and washed with sat. aq. Na₂CO₃ (2×50 mL). The organic layer was collected and dried over anhydrous Na₂SO₄ and concentrated under vacuum. This resulted in 1.21 g (crude) of the title compound as a dark yellow solid. MS-ESI: 292/294 (M+1).

### Step 11: 2,2-Difluoro-4-(prop-1-en-2-yl)-7,8-dihydro-6H-indeno[4,5-d][1,3]dioxol-5-amine

To a stirred solution of 4-bromo-2,2-difluoro-7,8-dihydro-6H-indeno[4,5-d][1,3]dioxol-5-amine (1.20 g, crude from last step) in dioxane (45 mL) and water (3 mL) under nitrogen was added 4,4,5,5-tetramethyl-2-(prop-1-en-2-yl)-1,3,2-dioxaborolane (362 mg, 2.15 mmol), Cs₂CO₃ (1.06 g, 3.24 mmol) and Pd(dppf)Cl₂ (237 mg, 0.32 mmol). The resulting solution was stirred for 16 h at 100° C. The solids were filtered out. The filtrate was diluted with 100 mL of water, extracted with 3×100 mL of EtOAc. The organic layers were combined and dried over anhydrous Na₂SO₄. The residue was purified by Prep-TLC with EtOAc/PE (1:5). This resulted in 320 mg (18.0% over two steps) of the title compound as dark yellow oil. MS-ESI: 254 (M+1).

### Step 12: 2,2-Difluoro-4-isopropyl-7,8-dihydro-6H-indeno[4,5-d][1,3]dioxol-5-amine

To a stirred solution of 2,2-difluoro-4-(prop-1-en-2-yl)-7,8-dihydro-6H-indeno[4,5-d][1,3]dioxol-5-amine (320 mg, 1.26 mmol) in MeOH (20 mL) under nitrogen was added Pd/C (10% wt., 300 mg). The flask was evacuated and refilled three times with hydrogen. Then, the resulting mixture was stirred for 16 h at RT under hydrogen with a balloon. The solids were filtered out. The filtrated was concentrated under vacuum. This resulted in 300 mg (93.4%) of the title compound as light yellow oil. MS-ESI: 256 (M+1).

### Step 13: 5-Bromo-2,2-difluoro-4-isopropyl-7,8-dihydro-6H-indeno[4,5-d][1,3]dioxole

To a solution of 2,2-difluoro-4-isopropyl-7,8-dihydro-6H-indeno[4,5-d][1,3]dioxol-5-amine (250 mg, 0.98 mmol) in ACN (15 mL) under nitrogen was added CuBr (422 mg, 2.94 mmol) in portions at RT. This was followed by the addition of tBuONO (121 mg, 1.18 mmol) dropwise with stirring at 0° C. The resulting solution was stirred for 20 min at 0° C. And then the resulting mixture was heated to at 60° C. with stirring for 3 h. The solids were filtered out. The residue was eluted from a silica gel column with PE (100%). This resulted in 90 mg (28.8%) of the title compound as yellow oil. ¹H NMR (300 MHz, CDCl₃) δ 3.60-3.35 (m, 1H), 3.15-2.85 (m, 4H), 2.25-2.05 (m, 2H), 1.35 (d, J=7.2 Hz, 6H).

### Step 14: Isopropyl 2-(2,2-difluoro-4-isopropyl-7,8-dihydro-6H-indeno[4,5-d][1,3]dioxol-5-yl)acetate

To a stirred solution of 5-bromo-2,2-difluoro-4-isopropyl-6H,7H,8H-indeno[4,5-d][1,3]dioxole (90 mg, 0.28 mmol) in THF (10 mL) under nitrogen was added Pd₂(dba)₃ (26 mg, 0.028 mmol), Xphos (14 mg, 0.028 mmol) at RT. Then, to the mixture was added (2-(tert-butoxy)-2-oxoethyl)zinc(II) bromide (728 mg, 2.80 mmol) in THF (5 mL) dropwise at RT. The resulting solution was stirred for 3 h at 65° C. The reaction was then quenched with 1 mL of sat. NH₄Cl (aq.) and was diluted with 30 mL of H₂O. The resulting solution was extracted with 2×20 mL of EtOAc. The organic layers were combined, dried over anhydrous Na₂SO₄ and concentrated under vacuum. The residue was eluted from a silica gel column with PE (100%). This resulted in 80 mg (80.8%) of the title compound as a yellow oil. ¹H NMR (400 MHz, CDCl₃) δ 3.56 (s, 2H), 3.14 (hept, J=7.0 Hz, 1H), 2.96 (t, J=7.5 Hz, 2H), 2.90 (t, J=7.5 Hz, 2H), 2.15 (quint, J=7.5 Hz, 2H), 1.45 (s, 9H), 1.33 (d, J=7.2 Hz, 6H).

### Step 15: 2-(2,2-Difluoro-4-isopropyl-7,8-dihydro-6H-indeno[4,5-d][1,3]dioxol-5-yl)acetic Acid

To a stirred solution of isopropyl 2-(2,2-difluoro-4-isopropyl-7,8-dihydro-6H-indeno[4,5-d][1,3]-dioxol-5-yl)acetate (80 mg, 0.226 mmol) in DCM (14 mL) was added TFA (4 mL) dropwise at 0° C. The resulting solution was stirred for 2 h at RT. The resulting solution was concentrated. This resulted in 90 mg (crude) of the title compound as a yellow solid which was used without further purification. MS-ESI. 297 (M−1).

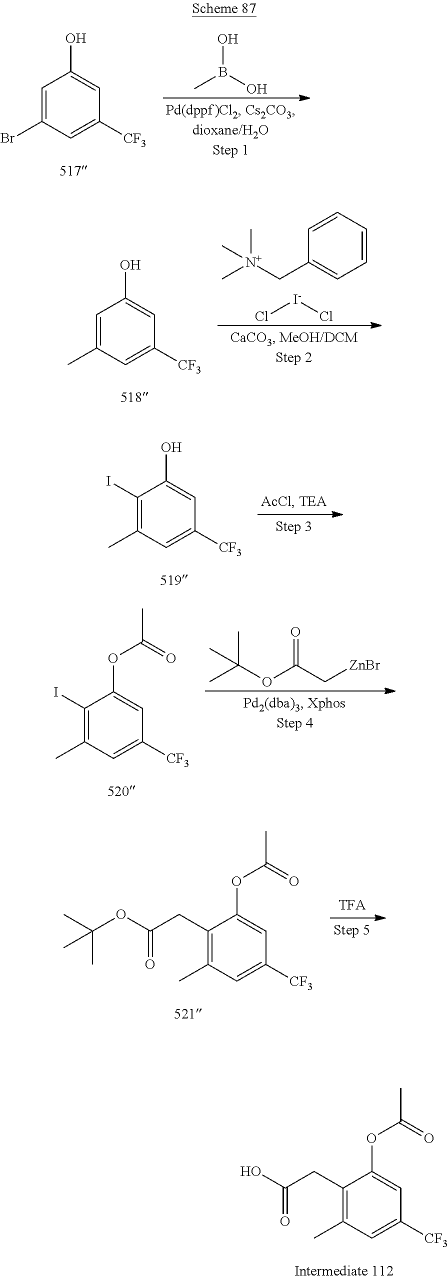

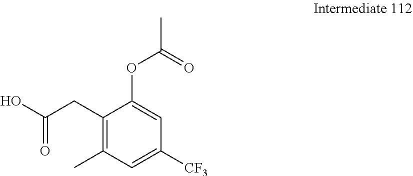 [SMILES: CC(=O)Oc1cc(C(F)(F)F)cc(C)c1CC(=O)O]

### 2-(2-Acetoxy-6-methyl-4-(trifluoromethyl)phenyl)acetic Acid

### Step 1: 3-Methyl-5-(trifluoromethyl)phenol

To a stirred solution of 3-bromo-5-(trifluoromethyl)phenol (5.0 g, 20.7 mmol) in dioxane (50 mL) and H₂O (5 mL) under nitrogen was added methylboronic acid (2.48 g, 41.4 mmol), Cs₂CO₃ (20.2 g, 62.1 mmol) and Pd(dppf)Cl₂ (1.52 g, 2.07 mmol). The resulting solution was stirred for 12 h at 90° C. The mixture was filtered through a Celite pad. The filtrate was concentrated under vacuum. The residue was eluted from a silica gel column with EtOAc/PE (1:1). This resulted in 2.3 g (63.2%) of the title compound as yellow oil. MS-ESI: 175 (M−1).

### Step 2: 2-Iodo-3-methyl-5-(trifluoromethyl)phenol

To a stirred solution of 3-methyl-5-(trifluoromethyl)phenol (2.30 g, 13.1 mmol) in MeOH (24 mL) and DCM (6 mL) under nitrogen was added N,N,N-trimethyl-1-phenylmethanaminium dichloroiodate (I) (4.56 g, 13.1 mmol) and CaCO₃ (3.93 g, 39.3 mmol). The resulting solution was stirred for 12 h at RT. The reaction was then quenched with 20 mL of sat. Na₂S₂O₃(aq), extracted with 3×50 mL of EtOAc. The organic layers were combined, dried over anhydrous Na₂SO₄ and concentrated under vacuum. The residue was eluted from a silica gel column with EtOAc/PE (1:2). This resulted in 800 mg (20.2%) of the title compound as yellow oil. MS-ESI: 175 (M−1).

### Step 3: 2-Iodo-3-methyl-5-(trifluoromethyl)phenyl Acetate

To a stirred solution of 2-iodo-3-methyl-5-(trifluoromethyl)phenol (800 mg, 2.65 mmol) in DCM (10 mL) was added TEA (803 mg, 7.95 mmol), followed by the addition of acetyl chloride (413 mg, 5.3 mmol) dropwise at 0° C. The resulting solution was stirred for 2 h at RT. The resulting solution was quenched with 30 mL of water/ice, extracted with 3×50 mL of DCM. The organic layers were combined, dried over anhydrous Na₂SO₄ and concentrated under vacuum. The residue was eluted from a silica gel column with EtOAc/PE (1:10). This resulted in 600 mg (65.8%) of the title compound as yellow oil. MS-ESI: 345 (M+1).

Steps 4-5 used similar procedures for converting compound 515″ to Intermediate 111 shown in Scheme 86 to afford Intermediate 112 from compound 520″. MS-ESI: 275 (M−1).

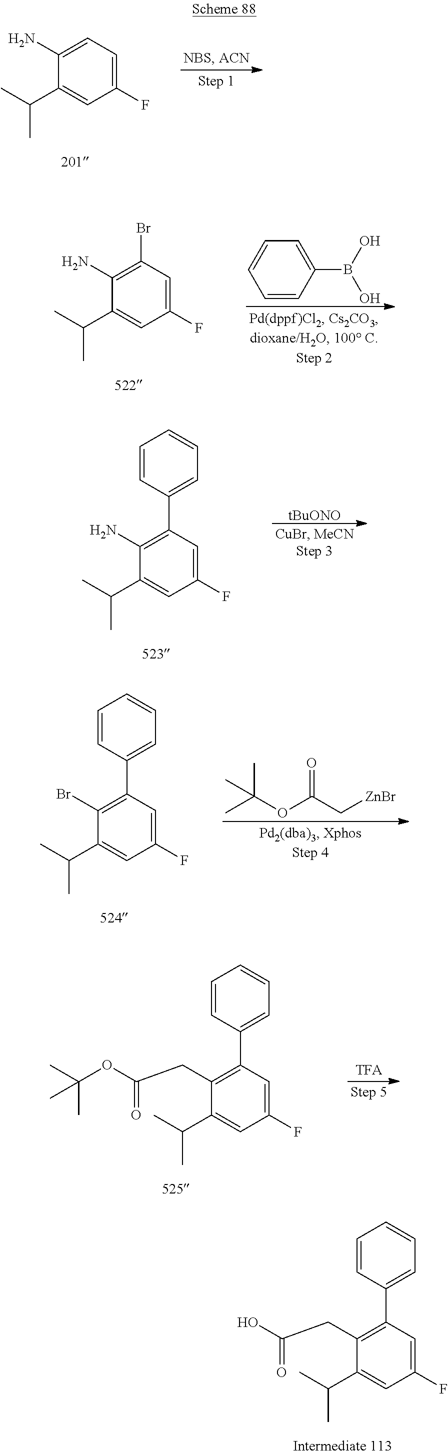

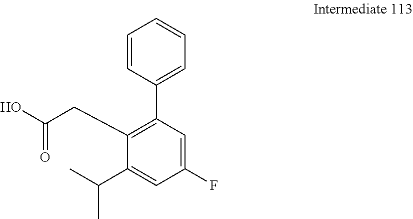 [SMILES: CC(C)c1cc(F)cc(-c2ccccc2)c1CC(=O)O]

### 2-(5-fluoro-3-isopropyl-[1,1′-biphenyl]-2-yl)acetic Acid

### Step 1: 2-Bromo-4-fluoro-6-isopropylaniline

To a stirred solution of 4-fluoro-2-isopropylaniline (1.0 g, 6.54 mmol) in ACN (10 mL) under nitrogen was added NBS (1.75 g, 9.81 mmol) in portions at 0° C. The resulting solution was stirred for 2 h at RT. The reaction was quenched with of sat. Na₂S₂O₃(aq.) and extracted with EtOAc (3×100 mL). The organic layers were combined, dried over anhydrous Na₂SO₄ and concentrated under vacuum. The residue was eluted from silica gel column with EtOAc/PE (1:10). This resulted in 1.2 g (78.9%) of the title compound as a dark oil. MS-ESI: 232/234 (M+1).

Steps 2-5 used similar procedures for converting compound 213″ to Intermediate 106 shown in Scheme 81 to afford Intermediate 113 from compound 522″. MS-ESI: 271 (M−1).

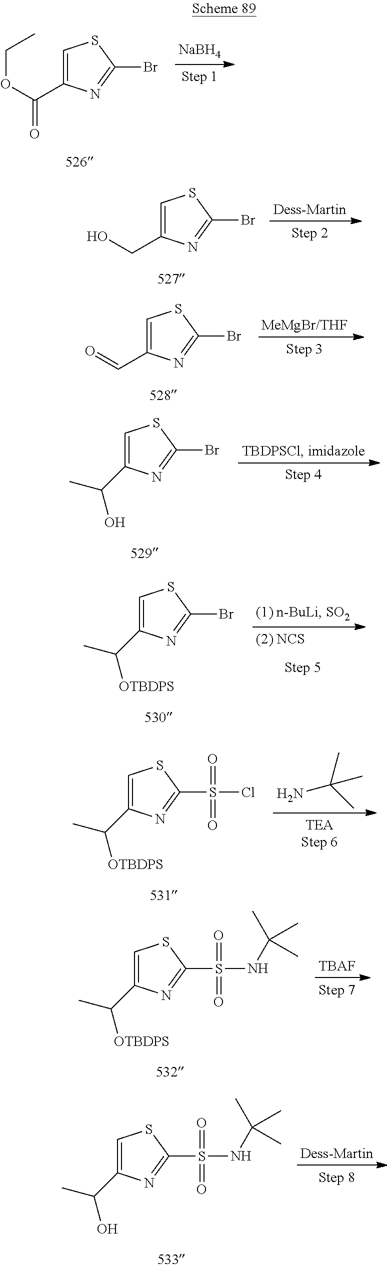

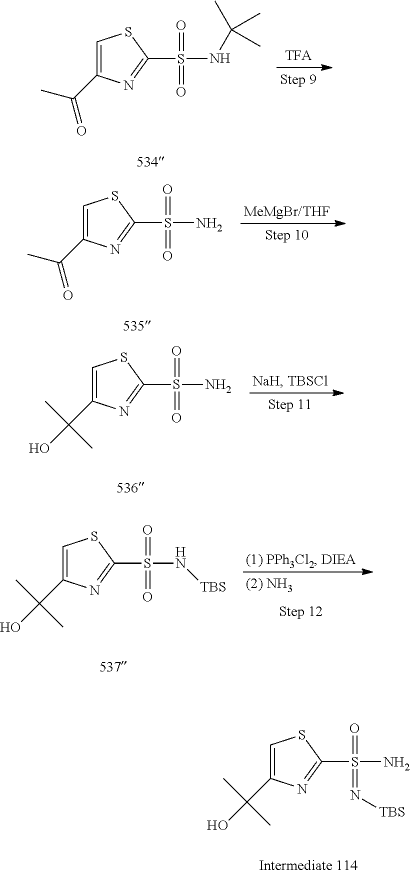

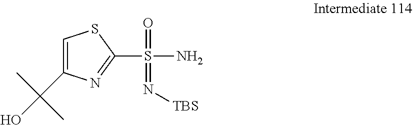

### N′-(tert-butyldimethylsilyl)-4-(2-hydroxypropan-2-yl)thiazole-2-sulfonimidamide

### Step 1: (2-Bromothiazol-4-yl)methanol

To a stirred solution of ethyl 2-bromothiazole-4-carboxylate (14 g, 59.3 mmol) in EtOH (200 mL) was added NaBH₄ (2.3 g, 60.5 mmol) in portions at 0° C. The resulting solution was stirred for 3 h at RT. The reaction mixture was quenched with 100 mL of water. Then extracted with 2×200 mL of DCM, the organic layers were combined and dried over anhydrous Na₂SO₄. The resulting solution was concentrated under vacuum. This resulted in 10.0 g (87%) of the title compound as colorless oil. MS-ESI. 196/194 (M+1).

### Step 2: 2-Bromothiazole-4-carbaldehyde

To a stirred solution of (2-bromothiazol-4-yl)methanol (10.0 g, 51.5 mmol) in DCM (100 mL) was added Dess-Martin (24.0 g, 56.6 mmol). The resulting solution was stirred for 2 h at RT. The resulting solution was concentrated under vacuum. The residue was eluted from silica gel with EtOAc/PE (1:20). This resulted in 8.0 g (81%) of the title compound as yellow oil. MS-ESI: 194/192 (M+1).

### Step 3: 1-(2-Bromothiazol-4-yl)ethan-1-ol

To a stirred solution of 2-bromothiazole-4-carbaldehyde (8.0 g, 41.7 mmol) in THF (100 mL) under nitrogen was added MeMgBr (3 M in THF, 15 mL) dropwise at 0° C. The resulting solution was stirred for 2 h at RT. The reaction mixture was quenched with 100 mL of NH₄Cl (sat.). Then extracted with 3×100 mL of EtOAc, the organic layers were combined and concentrated under vacuum. The residue was eluted from silica gel with EtOAc/PE (1:5). This resulted in 6.0 g (69%) of the title compound as brown oil. MS-ESI: 210/208 (M+1).

Steps 4-10 used similar procedures for converting compound 18 to compound 25 shown in Scheme 10A to afford compound 536″ from compound 529″. MS-ESI: 223 (M+1).

Steps 11-12 used similar procedures for converting compound 491″ to Intermediate 108 shown in Scheme 83 to afford Intermediate 114 from compound 536″. MS-ESI: 336 (M+1).

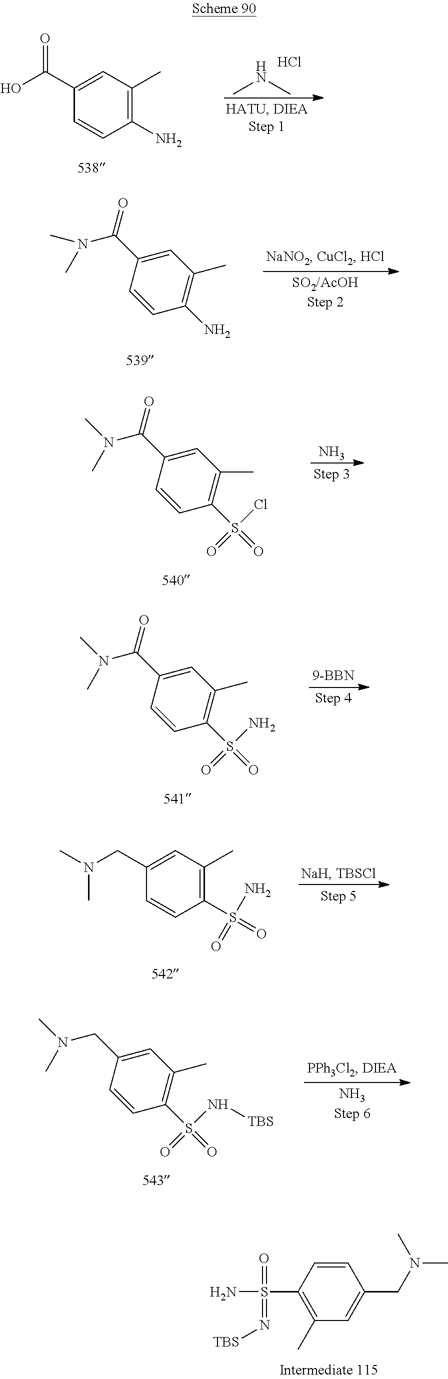

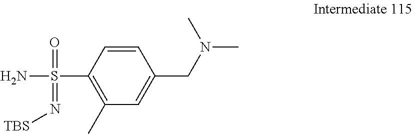

### N′-(tert-butyldimethylsilyl)-4-((dimethylamino)methyl)-2-methylbenzenesulfonimidamide

### Step 1: 4-Amino-N,N,3-trimethylbenzamide

To a stirred solution of 4-amino-3-methylbenzoic acid (10 g, 66.2 mmol) in THF (250 mL) was added DIEA (42.7 g, 331 mmol) and HATU (30.2 g, 79.4 mmol), then to the above solution was added dimethylamine hydrochloride (10.8 g, 132 mmol). The resulting solution was stirred for 1 h at RT. The reaction was quenched with 100 mL of water. Then extracted with 3×30 mL of EtOAc, the organic layers were combined and dried over anhydrous Na₂SO₄. The resulting solution was concentrated under vacuum. The residue was eluted from a silica gel column with EtOAc/PE (2:1). This resulted in 6.9 g (58.6%) of the title compound as an orange solid. MS-ESI: 179 (M+1).

Steps 2-6 used similar procedures for converting compound 32 to Intermediate 11 shown in Scheme 11 to afford Intermediate 115 from compound 539″. MS-ESI: 342 (M+1).

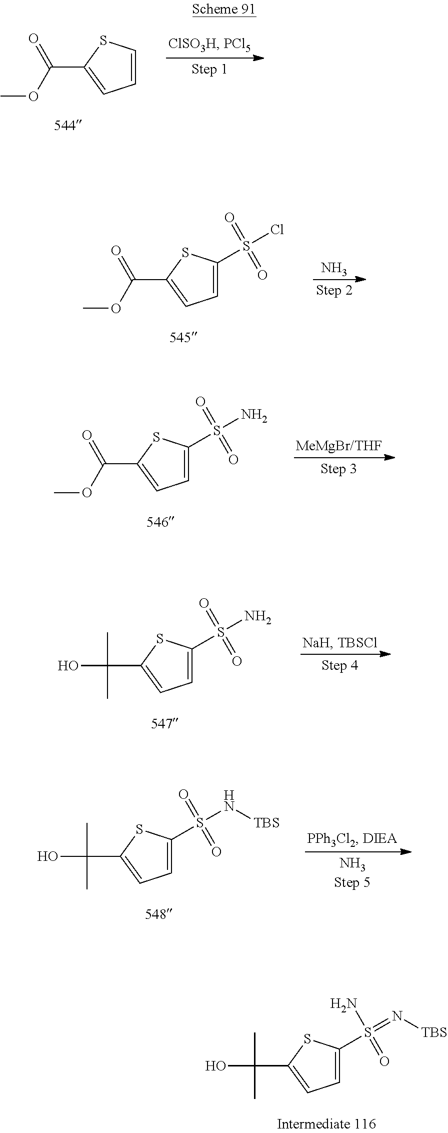

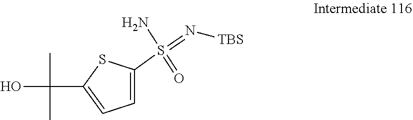

### N′-(tert-butyldimethylsilyl)-5-(2-hydroxypropan-2-yl)thiophene-2-sulfonimidamide

Steps 1-5 used similar procedures for converting compound 108″ to Intermediate 27 shown in Scheme 23 to afford Intermediate 116 from compound 544″. MS-ESI: 335 (M+1).

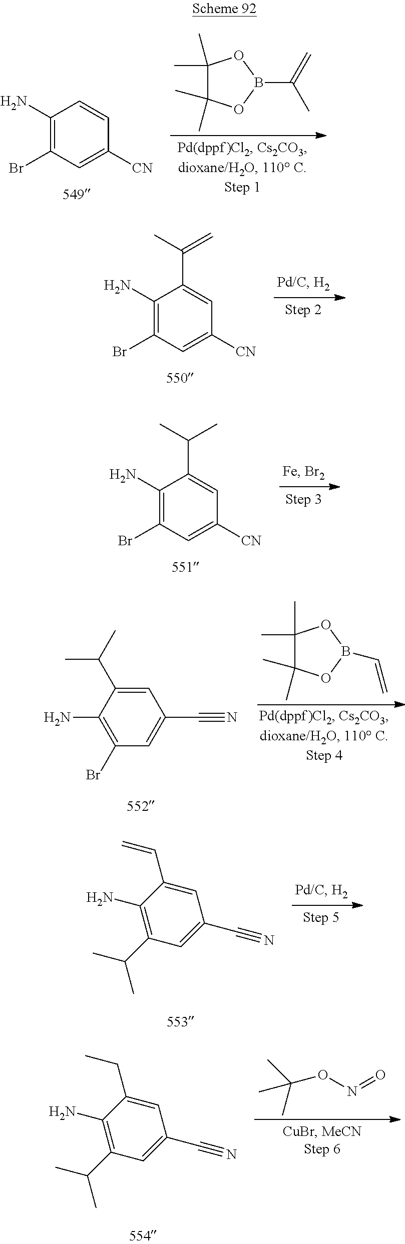

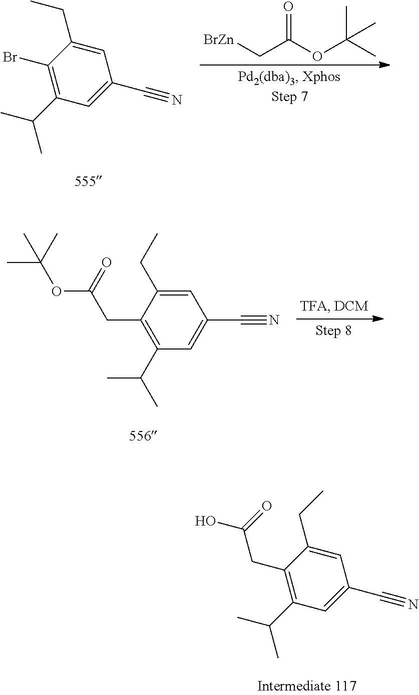

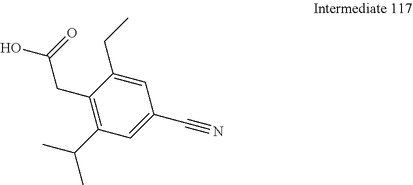 [SMILES: CCc1cc(C#N)cc(C(C)C)c1CC(=O)O]

### 2-(4-Cyano-2-ethyl-6-isopropylphenyl)acetic Acid

Steps 1-8 used similar procedures for converting compound 89 to Intermediate 23 shown in Scheme 19 to afford Intermediate 117 from compound 549″. MS-ESI. 230 (M−1).

### Example 1 (131)

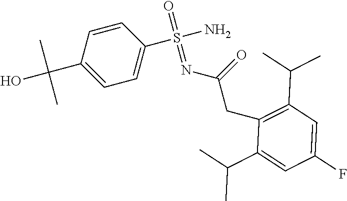 [SMILES: CC(C)c1cc(F)cc(C(C)C)c1CC(=O)N=S(N)(=O)c1ccc(C(C)(C)O)cc1]

### 2-(4-Fluoro-2,6-diisopropylphenyl)-N-(4-(2-hydroxypropan-2-yl)phenylsulfonimidoyl)acetamide (Scheme 1)

### Examples 2 (131b) and 3 (131a)

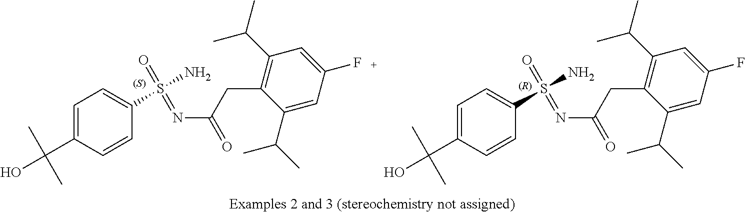

### (S)- and (R)-2-(4-Fluoro-2,6-diisopropylphenyl)-N-(4-(2-hydroxypropan-2-yl)phenylsulfonimidoyl)acetamide

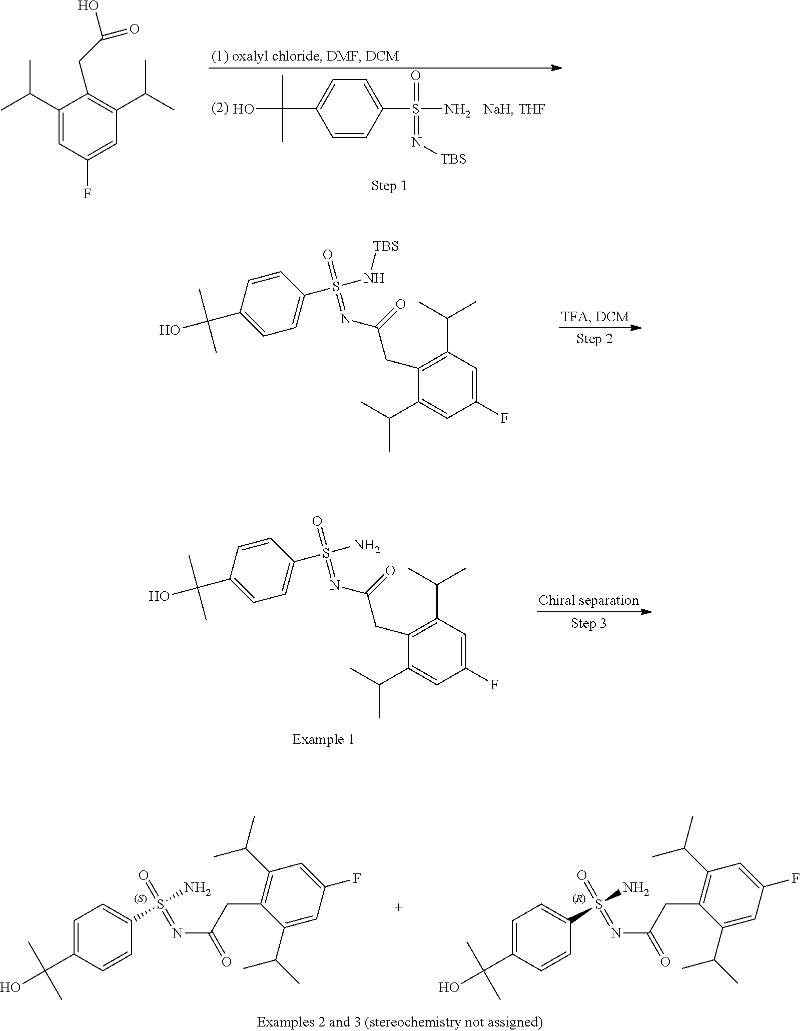

### Step 1: N-(tert-butyldimethylsilylamino-4-(2-hydroxypropan-2-yl)phenylhydrosulfonimidoyl)-2-(4-fluoro-2,6-diisopropylphenyl)acetamide

Into a 50-mL round-bottom flask was placed 2-(4-fluoro-2,6-diisopropylphenyl)acetic acid (73 mg, 0.31 mmol), DCM (2 mL), and DMF (0.05 mL). This was followed by the addition of oxalyl chloride (0.5 mL) dropwise with stirring at RT. The solution was stirred for 30 min at RT and then was concentrated under vacuum. Into a 50-mL round-bottom flask was placed a solution of N′-(tert-butyldimethylsilyl)-4-(2-hydroxypropan-2-yl)benzenesulfonimidamide (100 mg, 0.30 mmol) in THF (3 mL). This was followed by the addition of NaH (60% wt., 42 mg, 1.04 mmol) in portions at 0° C. The solution was stirred for 5 min at RT. Then to the above was added the solution of 2-(4-fluoro-2,6-diisopropylphenyl)acetyl chloride in THF (1 mL) prepared as shown above. The resulting solution was stirred for 1 h at RT, after which it was quenched by the addition of 5 mL of water and extracted with 2×5 mL of ethyl acetate. The combined organic layers were dried over anhydrous Na₂SO₄, and concentrated under vacuum. This resulted in 159 mg (96%) of the title compound as yellow crude oil. MS-ESI: 547.3 (M−1).

### Step 2: 2-(4-Fluoro-2,6-diisopropylphenyl)-N-(4-(2-hydroxypropan-2-yl)phenylsulfonimidoyl)acetamide

Into a 50-mL round-bottom flask was placed a solution of N-(tert-butyldimethylsilylamino-4-(2-hydroxylpropan-2-yl)phenylhydrosulfonimidoyl)-2-(4-fluoro-2,6-diisopropylphenyl)acetamide (159 mg, 0.29 mmol) in DCM (10 mL). Then TFA (0.2 mL) was added. The resulting solution was stirred for 1 h at RT and was concentrated under vacuum after that. The crude product was purified by Prep-HPLC using method E eluted with a gradient of 26˜50% ACN. This resulted in 13.0 mg (10%) of Example 1 as a white solid. MS-ESI: 435.3 (M+1). ¹H NMR (300 MHz, DMSO-d₆) δ 7.75 (d, J=8.7 Hz, 2H), 7.61 (d, J=8.7 Hz, 2H), 7.57 (s, 2H), 6.82 (d, J=10.5 Hz, 2H), 5.21 (s, 1H), 3.62-3.54 (m, 2H) 3.07-2.98 (m, 2H), 1.41 (s, 6H), 1.13 (d, J=6.9 Hz, 6H), 1.08 (d, J=6.9 Hz, 6H).

### Step 3: Chiral Separation

The product obtained as described in the previous step (90 mg) was resolved by Chiral-Prep-HPLC using the following conditions: Column, ChiralPak ID, 2*25 cm, 5 um; mobile phase, Hex and IPA (hold 40% IPA over 16 min); Flow rate, 20 mL/min; Detector, UV 254/220 nm. This resulted in 16.0 mg (front peak, enantiomer 1, 99% ee) of Example 2 as a white solid and 44.8 mg (second peak, enantiomer 2, 99% ee) of Example 3 as a light yellow solid. Absolute stereochemistry of these two isomers has not been assigned.

Example 2: MS-ESI: 435.1 (M+1). ¹H NMR (300 MHz, DMSO-d₆) δ 7.75 (d, J=8.4 Hz, 2H), 7.61 (d, J=9.0 Hz, 4H), 6.82 (d, J=10.5 Hz, 2H), 5.18 (s, 1H), 3.62-3.58 (m, 2H), 3.07-2.98 (m, 2H), 1.41 (s, 6H), 1.08 (d, J=6.9 Hz, 6H), 1.00 (d, J=6.6 Hz, 6H).

Example 3: MS-ESI: 435.1 (M+1). ¹H NMR (300 MHz, DMSO-d₆) δ 7.74 (d, J=8.7 Hz, 2H), 7.57 (d, J=8.4 Hz, 4H), 6.81 (d, J=10.5 Hz, 2H), 5.21 (s, 1H), 3.58-3.57 (m, 2H), 3.09-3.02 (m, 2H), 1.41 (s, 6H), 1.07 (d, J=6.6 Hz, 6H), 1.01 (d, J=6.6 Hz, 6H).

### Example 4 (129)

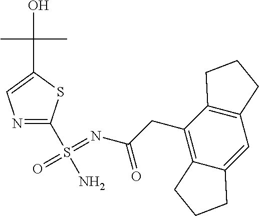 [SMILES: CC(C)(O)c1cnc(S(N)(=O)=NC(=O)Cc2c3c(cc4c2CCC4)CCC3)s1]

### 2-(1,2,3,5,6,7-Hexahydro-s-indacen-4-yl)-N-(5-(2-hydroxypropan-2-yl)thiazol-2-ylsulfonimidoyl)acetamide (Scheme 2)

### Examples 5 (129b) and 6 (129a)

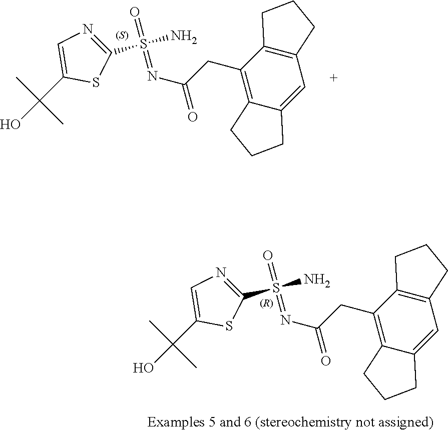

### (S)- and (R)-2-(1,2,3,5,6,7-Hexahydro-s-indacen-4-yl)-N-(5-(2-hydroxypropan-2-yl)thiazol-2-ylsulfonimidoyl)acetamide

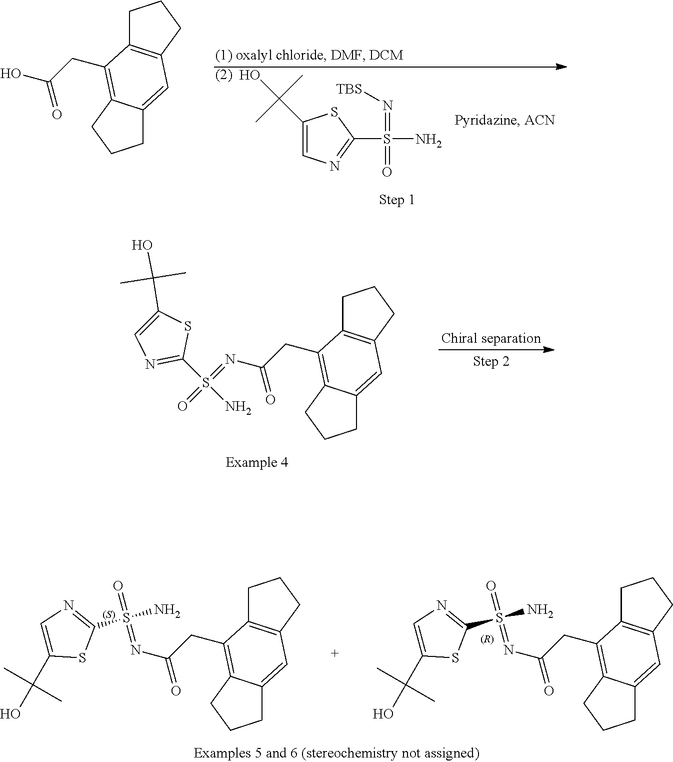

### Step 1: 2-(1,2,3,5,6,7-Hexahydro-s-indacen-4-yl)-N-(5-(2-hydroxypropan-2-yl)thiazol-2-ylsulfonimidoyl)acetamide

Into a 50-mL round-bottom flask was placed 2-(1,2,3,5,6,7-hexahydro-s-indacen-4-yl)acetic acid (100 mg, 0.46 mmol), DCM (2 mL), and DMF (0.05 mL). This was followed by the addition of oxalyl chloride (0.5 mL) dropwise with stirring at RT. The solution was stirred for 30 min at RT and then was concentrated under vacuum. The above mixture was diluted in ACN (3 mL). This was followed by the addition of a solution of pyridazine (37 mg, 0.46 mmol) in ACN (1 mL). The solution was stirred for 1 min at RT and then a solution of N′-(tert-butyldimethylsilyl)-5-(2-hydroxypropan-2-yl)thiazole-2-sulfonimidamide (154 mg, 0.46 mmol) in ACN (2 mL) was added. The resulting solution was stirred for 2 h at RT, after which it was concentrated under vacuum. The resulting residue was applied onto a silica gel column and eluted with ethyl acetate/petroleum ether (1:2 to 1:1). The crude product was purified by Prep-HPLC using method E eluted with a gradient of 26˜50% ACN. This resulted in 10 mg (5%) of Example 4 as a white solid. MS-ESI: 420.2 (M+1). ¹H NMR (300 MHz, DMSO-d₆) δ 7.84 (br s, 2H), 7.72 (s, 1H), 6.89 (s, 1H), 5.85 (s, 1H), 3.48-3.37 (m, 2H), 2.80-2.70 (m, 8H), 1.99-1.90 (m, 4H), 1.52-1.51 (m, 6H).

### Step 2: Chiral Separation

The product obtained as described in the previous step (40 mg) was resolved by Chiral-Prep-HPLC using the following conditions: ChiralPak IG, 2*25 cm, 5 um; mobile phase, Hex (0.1% TFA) and EtOH (hold 30% EtOH over 13.5 min); Flow rate, 20 mL/min; Detector, UV 254/220 nm. This resulted in 15.3 mg (front peak, enantiomer 1, 100% ee) of Example 5 as a white solid and 14.4 mg (second peak, enantiomer 2, 100% ee) of Example 6 as a white solid. Absolute stereochemistry of these two isomers has not been assigned.

Example 5: MS-ESI: 420.2 (M+1). ¹H NMR (300 MHz, DMSO-d₆) δ 8.12 (s, 2H), 7.80 (s, 1H), 6.90 (s, 1H), 5.93 (s, 1H), 3.48-3.40 (m, 2H), 2.80-2.50 (m, 8H), 2.08-1.89 (m, 4H), 1.54-1.52 (m, 6H).

Example 6: MS-ESI: 420.2 (M+1). ¹H NMR (300 MHz, DMSO-d₆) δ 8.12 (s, 2H), 7.80 (s, 1H), 6.91 (s, 1H), 5.93 (s, 1H), 3.48-3.40 (m, 2H), 2.80-2.69 (m, 8H), 1.99-1.90 (m, 4H), 1.54-1.52 (m, 6H).

### Example 7 (132)

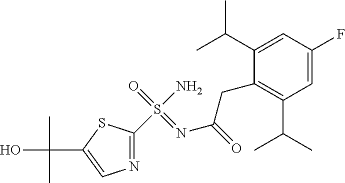 [SMILES: CC(C)c1cc(F)cc(C(C)C)c1CC(=O)N=S(N)(=O)c1ncc(C(C)(C)O)s1]

### 2-(4-Fluoro-2,6-diisopropylphenyl)-N-(5-(2-hydroxypropan-2-yl)thiazole-2-sulfonimidoyl)acetamide (Scheme 3)

### Examples 8 (132b) and 9 (132a)

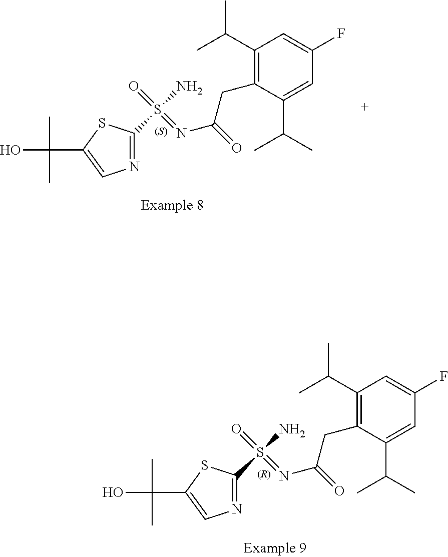

### (S)- and (R)-2-(4-fluoro-2,6-diisopropylphenyl)-N-(5-(2-hydroxypropan-2-yl)thiazole-2-sulfonimidoyl)acetamide

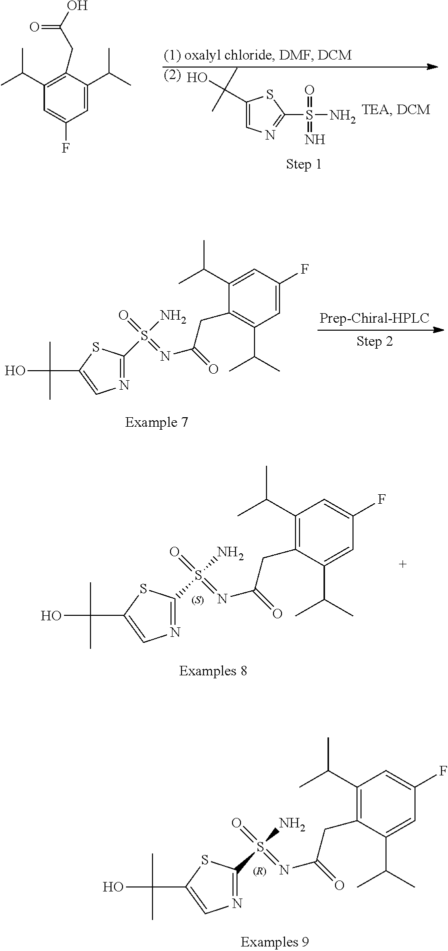

### Step 1: 2-(4-Fluoro-2,6-diisopropylphenyl)-N-(5-(2-hydroxypropan-2-yl)thiazole-2-sulfonimidoyl)acetamide

Into a 50-mL round-bottom flask was placed 2-(4-fluoro-2,6-diisopropylphenyl)acetic acid (108 mg, 0.45 mmol), DCM (3 mL), and DMF (0.05 mL). This was followed by the addition of oxalyl chloride (0.5 mL) dropwise with stirring at RT. The solution was stirred for 30 min at RT and then was concentrated under vacuum. To the above mixture, diluted in DCM (1 mL), was added to a solution of 5-(2-hydroxypropan-2-yl)thiazole-2-sulfonimidamide (60 mg, 0.27 mmol) and TEA (150 mg, 1.48 mmol) in DCM (3 mL) dropwise with stirring at RT. The resulting solution was stirred for 1 h at RT and was then concentrated under vacuum. The crude product was purified by Prep-HPLC using method E eluted with a gradient of 22˜44% ACN. This resulted in 2.1 mg (1%) of Example 7 as a white solid. MS-ESI. 442.3 (M+1). ¹H NMR (300 MHz, CD₃OD-d₄) δ 7.67 (s, 1H), 6.75 (d, J=10.5 Hz, 2H), 3.74 (s, 2H), 3.17-3.03 (m, 2H), 1.58 (s, 6H), 1.12 (d, J=6.8 Hz, 6H), 1.10 (d, J=6.8 Hz, 6H).

### Step 2: Chiral Separation

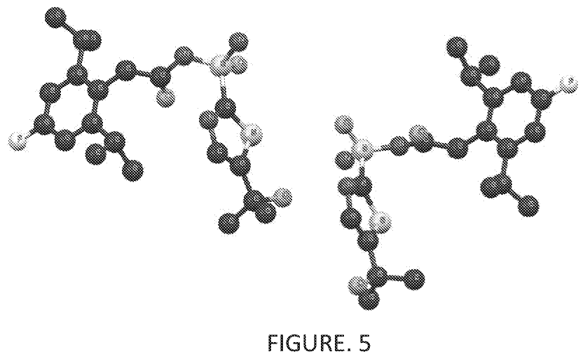

The product obtained in the previous step (10 mg) was resolved by Chiral-Prep-HPLC using following conditions: Column, ChiralPak ID, 2*25 cm, 5 um; mobile phase, Hex (0.1% FA) and EtOH (hold 20% EtOH in over 10 min); Flow rate, 20 mL/min; Detector, UV 254/220 nm. This resulted in 3.6 mg (front peak, enantiomer 1, 99% ee) of Example 8 as a yellow solid and 3.1 mg (second peak, enantiomer 2, 99% ee) of Example 9 as a yellow solid. Single crystal X-ray crystallographic analysis was performed on compound 132b. Table N below shows fractional atomic coordinates of compound 132b, and FIG. 5 shows ball and stick models of the asymmetrical unit containing two crystallographically independent molecules of compound 132b, with hydrogen atoms omitted for clarity. Based on these results, the absolute stereochemistry of compound 132b was assigned as (S) by single crystal X-ray structure determination. Therefore, compound 132a was assigned as the (R) isomer.

Example 8: MS-ESI: 442.2 (M+1). ¹H NMR (300 MHz, DMSO-d₆) δ 8.21 (br s, 2H), 7.75 (s, 1H), 6.84 (d, J=10.5 Hz, 2H), 5.88 (s, 1H), 3.65-3.63 (m, 2H), 3.10-2.97 (m, 2H), 1.51 (s, 6H), 1.09 (d, J=6.9 Hz, 6H), 1.04 (d, J=6.9 Hz, 6H).

Example 9: MS-ESI: 442.2 (M+1). ¹H NMR (300 MHz, DMSO-d₆) δ 7.73 (s, 1H), 6.83 (d, J=10.5 Hz, 2H), 5.87 (s, 1H), 3.63-3.59 (m, 2H), 3.10-3.03 (m, 2H), 1.51 (s, 6H), 1.09 (d, J=6.9 Hz, 6H), 1.04 (d, J=6.6 Hz, 6H).

TABLE N
Fractional Atomic Coordinates (×10⁴) and Equivalent Isotropic
Displacement Parameters (Å² × 10³) for Example 8. U_{eq} is defined as ⅓
of the trace of the orthogonalised U_{IJ} tensor.

| Atom | x | y | z | U(eq) |
|---|---|---|---|---|
| S3 | 6096(2) | 1631.9(12) | 6214.0(7) | 43.8(3) |
| S1 | 4186(3) | 6312.8(13) | 3775.3(8) | 49.4(4) |
| S4 | 7832(3) | 4132.7(15) | 5650.5(8) | 53.7(4) |
| S2 | 4909(3) | 9172.1(15) | 3606.7(9) | 64.2(5) |
| O4 | 7737(8) | 1198(4) | 5669(2) | 56.1(11) |
| O5 | 9907(8) | 1617(5) | 7085(2) | 62.4(12) |
| O1 | 2799(9) | 5566(4) | 4214(2) | 64.9(13) |
| F2 | 14863(9) | 1324(5) | 9826(3) | 88.3(14) |
| N4 | 6228(9) | 1015(5) | 6933(2) | 47.7(11) |
| O2 | 102(9) | 7078(6) | 2977(2) | 70.3(14) |
| F1 | −4874(11) | 7107(6) | 161(3) | 100.7(17) |
| N5 | 3489(10) | 1470(6) | 6026(3) | 54.6(13) |
| O3 | 2378(9) | 12418(4) | 4594(2) | 60.3(12) |
| N3 | 1905(9) | 8406(5) | 4478(2) | 46.5(11) |
| N1 | 4115(10) | 6318(5) | 3000(3) | 55.1(13) |
| N2 | 6903(11) | 5865(6) | 3894(3) | 60.1(15) |
| N6 | 5327(10) | 4040(5) | 6756(3) | 58.1(14) |
| O6 | 8678(13) | 6628(6) | 5147(3) | 86.0(18) |
| C3 | 3284(10) | 10337(6) | 4107(3) | 45.2(13) |
| C27 | 8106(11) | 1236(5) | 7308(3) | 46.2(14) |
| C29 | 9664(12) | 1054(6) | 8500(3) | 50.9(15) |
| C32 | 13181(13) | 1229(7) | 9385(3) | 59.3(17) |
| C24 | 7970(11) | 6826(6) | 5836(3) | 50.5(14) |
| C6 | 6192(12) | 11937(7) | 4004(4) | 63.5(18) |
| C30 | 10056(13) | 2297(6) | 8708(3) | 55.4(16) |
| C22 | 5791(13) | 5307(6) | 6651(3) | 59.0(17) |
| C28 | 7637(12) | 955(6) | 8054(3) | 51.2(15) |
| C23 | 7090(11) | 5560(6) | 6085(3) | 46.0(14) |
| C1 | 3454(11) | 7995(6) | 4013(3) | 46.6(13) |
| C2 | 1819(11) | 9753(6) | 4535(3) | 48.0(13) |
| C34 | 11159(13) | −85(6) | 8717(3) | 55.8(16) |
| C9 | 415(13) | 7159(7) | 1501(3) | 57.2(17) |
| C31 | 11805(14) | 2368(7) | 9156(4) | 63.5(18) |
| C10 | −915(15) | 8349(7) | 1276(4) | 65.2(19) |
| C33 | 12915(14) | 27(7) | 9168(3) | 60.3(17) |
| C4 | 3585(11) | 11787(6) | 4018(3) | 46.4(14) |
| C7 | 2090(13) | 6835(7) | 2692(3) | 55.1(16) |
| C8 | 2514(14) | 7140(8) | 1955(3) | 64.7(19) |
| C13 | −1943(16) | 5980(8) | 843(4) | 71(2) |
| C12 | −3143(15) | 7121(8) | 616(4) | 70(2) |
| C14 | −148(15) | 5971(7) | 1295(4) | 67(2) |
| C38 | 8604(16) | 3579(7) | 8461(4) | 73(2) |
| C5 | 2485(14) | 12376(7) | 3372(4) | 64.0(18) |
| C35 | 10957(16) | −1446(7) | 8477(4) | 72(2) |
| C11 | −2668(16) | 8307(8) | 821(4) | 76(2) |
| C21 | 6299(10) | 3342(6) | 6257(3) | 45.8(14) |
| C39 | 10220(20) | 4381(9) | 8022(5) | 99(3) |
| C40 | 7385(19) | 4354(9) | 9037(6) | 101(3) |
| C15 | 1040(20) | 4634(9) | 1552(6) | 95(3) |
| C26 | 9960(20) | 7080(12) | 6237(6) | 120(5) |
| C18 | −480(20) | 9672(9) | 1490(5) | 98(3) |
| C37 | 10310(40) | −2330(12) | 9044(6) | 153(6) |
| C25 | 5915(17) | 7965(8) | 5857(6) | 92(3) |
| C17 | −680(30) | 3869(11) | 1918(6) | 132(5) |
| C16 | 2410(20) | 3878(10) | 979(8) | 124(5) |
| C19 | 80(40) | 10550(14) | 950(8) | 198(10) |
| C36 | 13120(30) | −2025(15) | 8091(10) | 199(10) |
| C20 | −2400(50) | 10316(15) | 1931(10) | 292(18) |

### Example 10 (134)

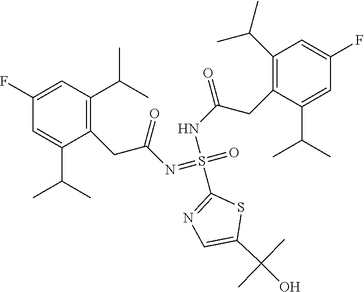 [SMILES: CC(C)c1cc(F)cc(C(C)C)c1CC(=O)N=S(=O)(NC(=O)Cc1c(C(C)C)cc(F)cc1C(C)C)c1ncc(C(C)(C)O)s1]

### N,N′-(2-hydroxypropan-2-yl)thiazol-2-ylsulfinyl)bis(2-(4-fluoro-2,6-diisopropylphenyl)acetamide) (Scheme 4)

Into a 50-mL round-bottom flask was placed 2-(4-fluoro-2,6-diisopropylphenyl)acetic acid (242 mg, 1.02 mmol), DCM (3 mL), and DMF (0.05 mL). This was followed by the addition of oxalyl chloride (0.5 mL) dropwise with stirring at RT. The solution was stirred for 30 min at RT and then was concentrated under vacuum. To the above mixture, diluted in DCM (2 mL), was added to a solution of 5-(2-hydroxypropan-2-yl)thiazole-2-sulfonimidamide (220 mg, 0.99 mmol) and TEA (400 mg, 3.95 mmol) in DCM (3 mL) dropwise with stirring at RT. The resulting solution was stirred for 1 h at RT and was then concentrated under vacuum. The crude product was purified by Prep-HPLC using method E eluted with a gradient of 63˜65% ACN. This resulted in 51.6 mg (8%) of Example 10 as a white solid. MS-ESI: 660.5 (M−1). ¹H NMR (300 MHz, CD₃OD-d₄) δ 7.59 (s, 1H), 5.75 (d, J=10.2 Hz, 4H), 3.82 (s, 4H), 3.16-3.06 (m, 4H), 1.59 (s, 6H), 1.32-1.13 (m, 24H).

### Example 11 (137)

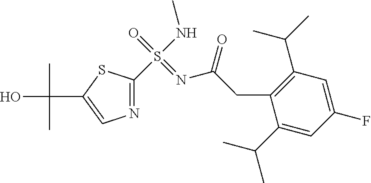 [SMILES: CNS(=O)(=NC(=O)Cc1c(C(C)C)cc(F)cc1C(C)C)c1ncc(C(C)(C)O)s1]

### 2-(4-Fluoro-2,6-diisopropylphenyl)-N-(5-(2-hydroxypropan-2-yl)-N-methylthiazole-2-sulfonimidoyl)acetamide (Scheme 5)

Into a 50-mL round-bottom flask was placed 2-(4-fluoro-2,6-diisopropylphenyl)acetic acid (233 mg, 0.98 mmol), DCM (3 mL), and DMF (0.05 mL). This was followed by the addition of oxalyl chloride (0.5 mL) dropwise with stirring at RT. The solution was stirred for 30 min at RT and then was concentrated under vacuum. To the above mixture, diluted in DCM (2 mL), was added to a solution of 5-(2-hydroxypropan-2-yl)-N′-methylthiazole-2-sulfonimidamide (230 mg, 0.98 mmol) and TEA (400 mg, 3.95 mmol) in DCM (3 mL) dropwise with stirring at RT. The resulting solution was stirred for 1 h at RT and then was concentrated under vacuum. The crude product was purified by Prep-HPLC using method E eluted with a gradient of 20˜80% ACN. This resulted in 17.7 mg (4%) of Example 11 as a light yellow solid. MS-ESI: 456.2 (M+1). ¹H NMR (300 MHz, DMSO-d₆) major tautomer δ 8.34 (s, 1H), 7.85 (s, 1H), 6.86 (d, J=10.5 Hz, 2H), 5.94 (s, 1H), 3.71 (d, J=3.6 Hz, 2H), 3.14-3.02 (m, 2H), 2.55 (s, 3H), 1.52 (s, 6H), 1.13 (d, J=4.5 Hz, 6H), 1.09 (d, J=4.5 Hz, 6H).

### Example 12 (136)

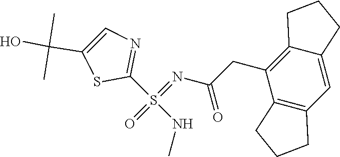 [SMILES: CNS(=O)(=NC(=O)Cc1c2c(cc3c1CCC3)CCC2)c1ncc(C(C)(C)O)s1]

### 2-(1,2,3,5,6,7-Hexahydro-s-indacen-4-yl)-N-(5-(2-hydroxypropan-2-yl)-N-methylthiazole-2-sulfonimidoyl)acetamide (Scheme 6)

Into a 50-mL round-bottom flask was placed 2-(1,2,3,5,6,7-hexahydro-s-indacen-4-yl)acetic acid (156 mg, 0.72 mmol), DCM (3 mL), and DMF (0.05 mL). This was followed by the addition of oxalyl chloride (0.5 mL) dropwise with stirring at RT. The solution was stirred for 30 min at RT and then was concentrated under vacuum. To the above mixture, diluted in DCM (2 mL), was added to a solution of 5-(2-hydroxypropan-2-yl)-N′-methylthiazole-2-sulfonimidamide (170 mg, 0.72 mmol) and DBU (370 mg, 2.43 mmol) in DCM (3 mL) dropwise with stirring at RT. The resulting solution was stirred for 2 h at RT and then was quenched by the addition of 10 mL of water. The resulting solution was extracted with 3×10 mL of ethyl acetate, dried over anhydrous Na₂SO₄, and then concentrated under vacuum. The crude product was purified by Prep-HPLC using method E eluted with a gradient of 5˜15% ACN. This resulted in 11.3 mg (4%) of the title compound as a light yellow solid. MS-ESI. 434.3 (M+1). ¹H NMR (300 MHz, CD₃OD-d₄) major tautomer δ 7.75 (s, 1H), 6.89 (s, 1H), 3.56 (s, 2H), 2.83-2.73 (m, 8H), 2.61 (s, 3H), 2.06-1.95 (m, 4H), 1.59 (s, 6H).

TABLE 8

| | | | | LC-MS |
|---|---|---|---|---|
| Ex. # | Cmpd # | Structure | IUPAC Name | [M + H]⁺ |
| 13 | 133 | 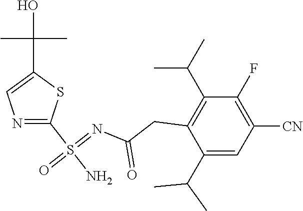 [SMILES: CC(C)c1cc(C#N)c(F)c(C(C)C)c1CC(=O)N=S(N)(=O)c1ncc(C(C)(C)O)s1] | 2-(4-cyano-3-fluoro-2,6-diisopropylphenyl)-N-(5-(2- hydroxypropan-2-yl)thiazol-2-ylsulfonimidoyl) acetamide | 467.2 |

Example in the following table was prepared using similar conditions as described in Example 1 and Scheme 1 from appropriate starting materials.

TABLE 9

| | | | | LC-MS |
|---|---|---|---|---|
| Ex. # | Cmpd # | Structure | IUPAC Name | [M + H]⁺ |
| 14 | 141 | 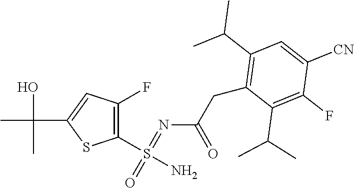 [SMILES: CC(C)c1cc(C#N)c(F)c(C(C)C)c1CC(=O)N=S(N)(=O)c1sc(C(C)(C)O)cc1F] | 2-(4-cyano-3-fluoro-2,6- diisopropylphenyl)-N-(3-fluoro-5- (2-hydroxypropan-2-yl)thiophen-2- ylsulfonimidoyl)acetamide | 484.2 |
| 15 | 139 | 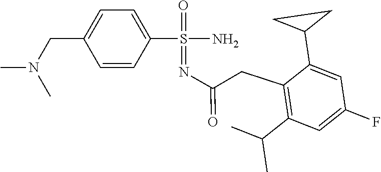 [SMILES: CC(C)c1cc(F)cc(C2CC2)c1CC(=O)N=S(N)(=O)c1ccc(CN(C)C)cc1] | 2-(2-cyclopropyl-4-fluoro- 6-isopropylphenyl)-N-(4- ((dimethylamino)methyl) phenylsulfonimidoyl)acetamide | 432.2 |
| 16 | 138 | 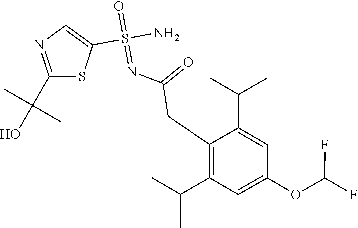 [SMILES: CC(C)c1cc(OC(F)F)cc(C(C)C)c1CC(=O)N=S(N)(=O)c1cnc(C(C)(C)O)s1] | 2-(4-(difluoromethoxy)-2,6- diisopropylphenyl)-N-(2-(2- hydroxypropan-2-yl)thiazol- 5-ylsulfonimidoyl)acetamide | 490.3 |
| 17 | 140 | 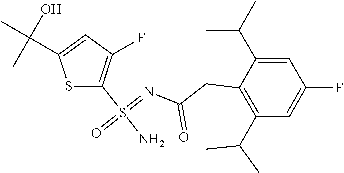 [SMILES: CC(C)c1cc(F)cc(C(C)C)c1CC(=O)N=S(N)(=O)c1sc(C(C)(C)O)cc1F] | 2-(4-fluoro-2,6- diisopropylphenyl)-N-(3-fluoro-5-(2- hydroxypropan-2-yl)thiophen- 2-ylsulfonimidoyl)acetamide | 459.2 |
| 18 | 142 | 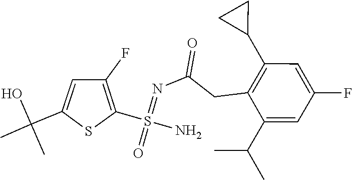 [SMILES: CC(C)c1cc(F)cc(C2CC2)c1CC(=O)N=S(N)(=O)c1sc(C(C)(C)O)cc1F] | 2-(2-cyclopropyl-4-fluoro-6- isopropylphenyl)-N-(3-fluoro-5- (2-hydroxypropan-2-yl)thiophen-2- ylsulfonimidoyl)acetamide | 457.2 |
| 19 | 143 | 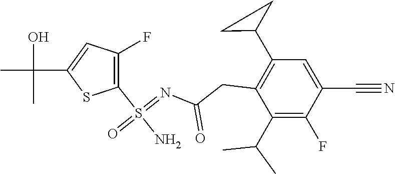 [SMILES: CC(C)c1c(F)c(C#N)cc(C2CC2)c1CC(=O)N=S(N)(=O)c1sc(C(C)(C)O)cc1F] | 2-(4-cyano-6-cyclopropyl-3- fluoro-2-isopropylphenyl)-N-(3- fluoro-5-(2-hydroxypropan-2-yl)thiophen-2- ylsulfonimidoyl)acetamide | 482.2 |
| 20 | 197 | 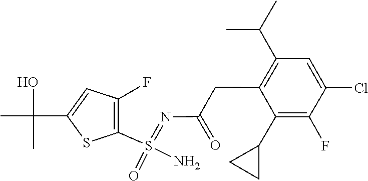 [SMILES: CC(C)c1cc(Cl)c(F)c(C2CC2)c1CC(=O)N=S(N)(=O)c1sc(C(C)(C)O)cc1F] | 2-(4-chloro-2-cyclopropyl-3- fluoro-6-isopropylphenyl)-N- (3-fluoro-5-(2-hydroxypropan- 2-yl)thiophen-2- ylsulfonimidoyl)acetamide | 491.2 |
| 21 | 101 | 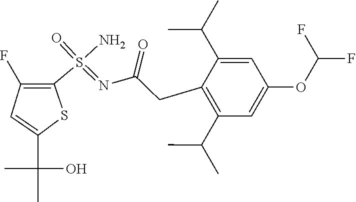 [SMILES: CC(C)c1cc(OC(F)F)cc(C(C)C)c1CC(=O)N=S(N)(=O)c1sc(C(C)(C)O)cc1F] | 2-(4-(difluoromethoxy)-2,6- diisopropylphenyl)-N-(3-fluoro- 5-(2-hydroxypropan-2-yl)thiophen-2- ylsulfonimidoyl)acctamide | 507.2 |
| 22 | 152 | 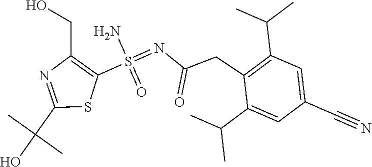 [SMILES: CC(C)c1cc(C#N)cc(C(C)C)c1CC(=O)N=S(N)(=O)c1sc(C(C)(C)O)nc1CO] | 2-(4-cyano-2,6-diisopropylphenyl)- N-(4-(hydroxymethyl)-2- (2-hydroxypropan-2-yl)thiazol- 5-ylsulfonimidoyl)acetamide | 479.2 |
| 23 | 150 | 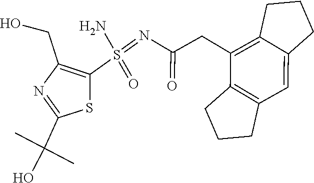 [SMILES: CC(C)(O)c1nc(CO)c(S(N)(=O)=NC(=O)Cc2c3c(cc4c2CCC4)CCC3)s1] | 2-(1,2,3,5,6,7-hexahydros- indacen-4-yl)-N-(4- (hydroxymethyl)-2-(2- hydroxypropan-2-yl)thiazol-5- ylsulfonimidoyl)acetamide | 450.2 |
| 24 | 148 | 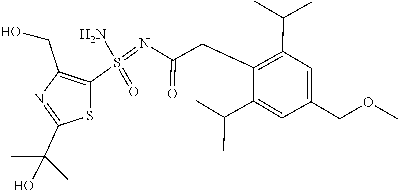 [SMILES: COCc1cc(C(C)C)c(CC(=O)N=S(N)(=O)c2sc(C(C)(C)O)nc2CO)c(C(C)C)c1] | 2-(2,6-diisopropyl-4- (methoxymethyl)phenyl)-N-(4- (hydroxymethyl)-2-(2- hydroxypropan-2-yl)thiazole-5- sulfonimidoyl)acetamide | 498.2 |
| 25 | 147 | 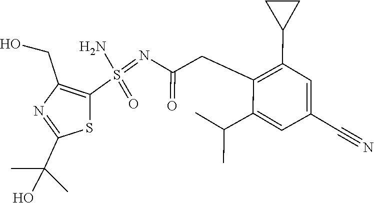 [SMILES: CC(C)c1cc(C#N)cc(C2CC2)c1CC(=O)N=S(N)(=O)c1sc(C(C)(C)O)nc1CO] | 2-(4-cyano-2-cyclopropyl- 6-isopropylphenyl)-N-(4- (hydroxymethyl)-2-(2- hydroxypropan-2-yl)thiazole-5- sulfonimidoyl)acetamide | 477.2 |
| 26 | 113 | 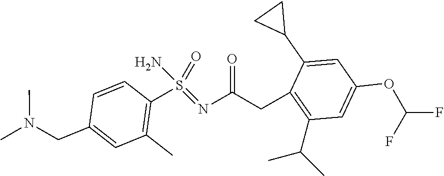 [SMILES: Cc1cc(CN(C)C)ccc1S(N)(=O)=NC(=O)Cc1c(C(C)C)cc(OC(F)F)cc1C1CC1] | 2-(2-cyclopropyl-4- (difluoromethoxy)-6-isopropylphenyl)- N-(4-((dimethylamino)methyl)-2- methylphenylsulfonimidoyl)acetamide | 494.2 |
| 27 | 151 | 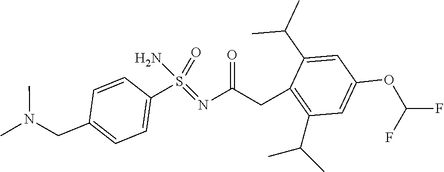 [SMILES: CC(C)c1cc(OC(F)F)cc(C(C)C)c1CC(=O)N=S(N)(=O)c1ccc(CN(C)C)cc1] | 2-(4-(difluoromethoxy)-2,6- diisopropylphenyl)-N-(4- ((dimethylamino)methyl) phenylsulfonimidoyl)acetamide | 482.2 |
| 28 | 114 | 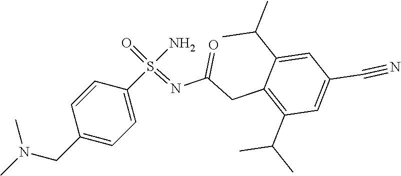 [SMILES: CC(C)c1cc(C#N)cc(C(C)C)c1CC(=O)N=S(N)(=O)c1ccc(CN(C)C)cc1] | 2-(4-cyano-2,6-diisopropylphenyl)-N-(4- ((dimethylamino)methyl) phenylsulfonimidoyl)acetamide | 441.2 |
| 29 | 112 | 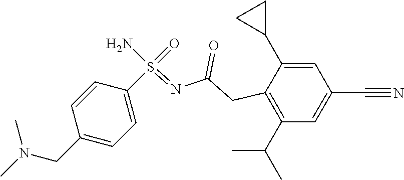 [SMILES: CC(C)c1cc(C#N)cc(C2CC2)c1CC(=O)N=S(N)(=O)c1ccc(CN(C)C)cc1] | 2-(4-cyano-2-cyclopropyl- 6-isopropylphenyl)-N-(4- ((dimethylamino)methyl) phenylsulfonimidoyl)acetamide | 439.2 |
| 30 | 116 | 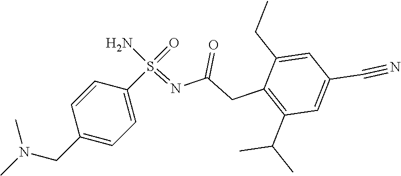 [SMILES: CCc1cc(C#N)cc(C(C)C)c1CC(=O)N=S(N)(=O)c1ccc(CN(C)C)cc1] | 2-(4-cyano-2-ethyl-6- isopropylphenyl)-N-(4- ((dimethylamino)methyl) phenylsulfonimidoyl)acetamide | 427.2 |
| 31 | 105 | 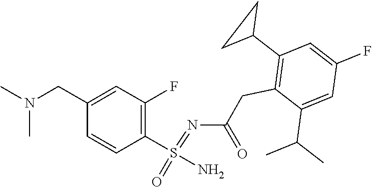 [SMILES: CC(C)c1cc(F)cc(C2CC2)c1CC(=O)N=S(N)(=O)c1ccc(CN(C)C)cc1F] | 2-(2-cyclopropyl-4-fluoro-6- isopropylphenyl)-N-(4- ((dimethylamino)methyl)-2- fluorophenylsulfonimidoyl)acetamide | 450.2 |
| 32 | 117 | 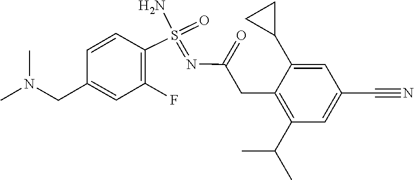 [SMILES: CC(C)c1cc(C#N)cc(C2CC2)c1CC(=O)N=S(N)(=O)c1ccc(CN(C)C)cc1F] | 2-(4-cyano-2-cyclopropyl- 6-isopropylphenyl)-N-(4- ((dimethylamino)methyl)-2- fluorophenylsulfonimidoyl)acetamide | 457.2 |
| 33 | 109 | 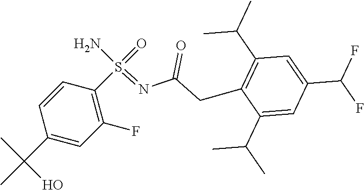 [SMILES: CC(C)c1cc(C(F)F)cc(C(C)C)c1CC(=O)N=S(N)(=O)c1ccc(C(C)(C)O)cc1F] | 2-(4-(difluoromethyl)-2,6- diisopropylphenyl)-N-(2-fluoro-4- (2-hydroxypropan-2-yl) phenylsulfonimidoyl)acetamide | 485.2 |
| 34 | 146 | 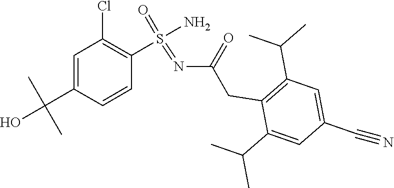 [SMILES: CC(C)c1cc(C#N)cc(C(C)C)c1CC(=O)N=S(N)(=O)c1ccc(C(C)(C)O)cc1Cl] | N-(2-chloro-4-(2-hydroxypropan- 2-yl)phenylsulfonimidoyl)- 2-(4-cyano-2,6- diisopropylphenyl)acetamide | 476.2 |
| 35 | 110 | 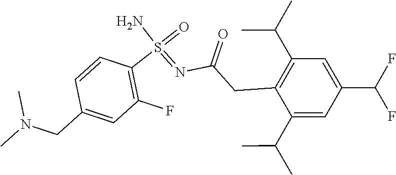 [SMILES: CC(C)c1cc(C(F)F)cc(C(C)C)c1CC(=O)N=S(N)(=O)c1ccc(CN(C)C)cc1F] | 2-(4-(difluoromethyl)-2,6- diisopropylphenyl)-N-(4- ((dimethylamino)methyl)-2- fluorophenylsulfonimidoyl)acetamide | 484.2 |
| 36 | 126 | 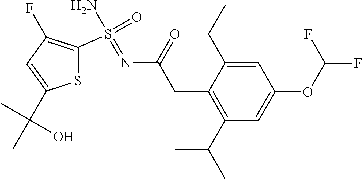 [SMILES: CCc1cc(OC(F)F)cc(C(C)C)c1CC(=O)N=S(N)(=O)c1sc(C(C)(C)O)cc1F] | 2-(4-(difluoromethoxy)-2-ethyl- 6-isopropylphenyl)-N-(3- fluoro-5-(2-hydroxypropan- 2-yl)thiophene-2- sulfonimidoyl)acetamide | 493.1 |
| 37 | 104 | 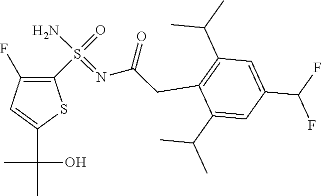 [SMILES: CC(C)c1cc(C(F)F)cc(C(C)C)c1CC(=O)N=S(N)(=O)c1sc(C(C)(C)O)cc1F] | 2-(4-(difluoromethyl)-2,6- diisopropylphenyl)-N-(3-fluoro-5- (2-hydroxypropan-2-yl)thiophene-2- sulfonimidoyl)acetamide | 491.2 |
| 38 | 130 | 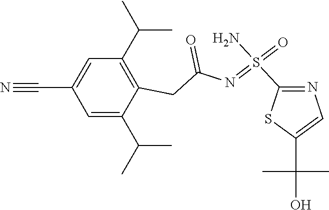 [SMILES: CC(C)c1cc(C#N)cc(C(C)C)c1CC(=O)N=S(N)(=O)c1ncc(C(C)(C)O)s1] | 2-(4-cyano-2,6-diisopropylphenyl)- N-(5-(2-hydroxypropan- 2-yl)thiazole-2-sulfonimidoyl)acetamide | 449.2 |
| 39 | 107 | 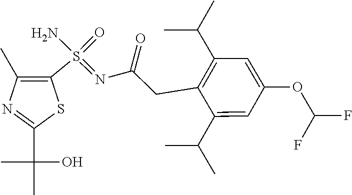 [SMILES: Cc1nc(C(C)(C)O)sc1S(N)(=O)=NC(=O)Cc1c(C(C)C)cc(OC(F)F)cc1C(C)C] | 2-(4-(difluoromethoxy)-2,6- diisopropylphenyl)-N-(2-(2- hydroxypropan-2-yl)-4-methylthiazole-5- sulfonimidoyl)acetamide | 504.2 |
| 40 | 106 | 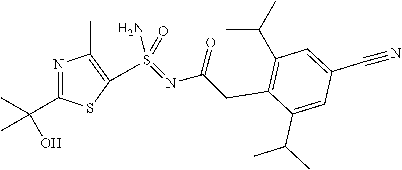 [SMILES: Cc1nc(C(C)(C)O)sc1S(N)(=O)=NC(=O)Cc1c(C(C)C)cc(C#N)cc1C(C)C] | 2-(4-cyano-2,6-diisopropylphenyl)- N-(2-(2-hydroxypropan- 2-yl)-4-methylthiazole-5- sulfonimidoyl)acetamide | 463.2 |

Examples in the following table were prepared using similar conditions as described in Example 4 and Scheme 2 from appropriate starting materials.

TABLE 10

| | | | | LC-MS |
|---|---|---|---|---|
| Ex. # | Cmpd # | Structure | IUPAC Name | [M + H]⁺ |
| 41 | 135 | 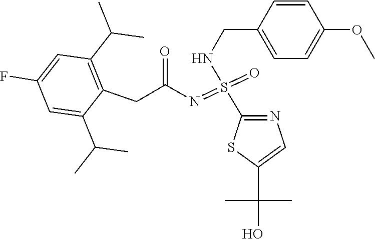 [SMILES: COc1ccc(CNS(=O)(=NC(=O)Cc2c(C(C)C)cc(F)cc2C(C)C)c2ncc(C(C)(C)O)s2)cc1] | 2-(4-fluoro-2,6-diisopropylphenyl)-N-(5-(2- hydroxypropan-2-yl)-N-(4-methoxybenzyl)thiazole-2- sulfonimidoyl)acetamide | 562.5 |

Example in the following table was prepared using similar conditions as described in Example 11 and Scheme 5 from appropriate starting materials.

TABLE 11

| | | | | | | LC-MS |
|---|---|---|---|---|---|---|
| Ex. | Cmpd | | | | | [M + |
| # | # | Structure | IUPAC Name | Column | Eluents | H]⁺ |
| 42 | 133b or 133a | 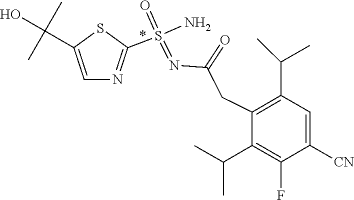 [SMILES: CC(C)c1cc(C#N)c(F)c(C(C)C)c1CC(=O)N=S(N)(=O)c1ncc(C(C)(C)O)s1] | (S)-or (R)- 2-(4-cyano-3-fluoro-2,6- diisopropylphenyl)-N-(5- (2-hydroxypropan-2-yl) thiazol-2-ylsulfonimidoyl) acetamide | ChiralPak IC, 2*25 cm, 5 um | 50% IPA in CO₂ | 467.2 |
| 43 | 133a or 133b | 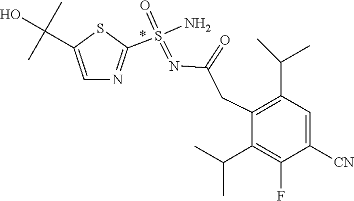 [SMILES: CC(C)c1cc(C#N)c(F)c(C(C)C)c1CC(=O)N=S(N)(=O)c1ncc(C(C)(C)O)s1] | (R)-or (S)- 2-(4-cyano-3-fluoro-2,6- diisopropylphenyl)-N-(5- (2-hydroxypropan-2-yl) thiazol-2-ylsulfonimidoyl) acetamide | ChiralPak IC, 2*25 cm, 5 um | 50% IPA in CO₂ | 467.2 |
| 44 | 141b or 141a | 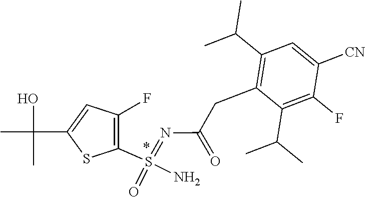 [SMILES: CC(C)c1cc(C#N)c(F)c(C(C)C)c1CC(=O)N=S(N)(=O)c1sc(C(C)(C)O)cc1F] | (S)- or (R)- 2-(4-cyano-3-fluoro-2,6- diisopropylphenyl)-N-(3- fluoro-5-(2-hydroxypropan- 2-yl)thiophen-2- ylsulfonimidoyl)acetamide | ChiralPak ID, 2*25 cm, 5 um | 30% EtOH in Hex (1% TFA) | 484.2 |
| 45 | 141a or 141b | 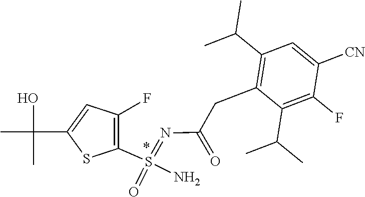 [SMILES: CC(C)c1cc(C#N)c(F)c(C(C)C)c1CC(=O)N=S(N)(=O)c1sc(C(C)(C)O)cc1F] | (R)- or (S)- 2-(4-cyano-3-fluoro-2,6- diisopropylphenyl)-N-(3- fluoro-5-(2-hydroxypropan- 2-yl)thiophen-2- ylsulfonimidoyl)acetamide | ChiralPak ID, 2*25 cm, 5 um | 30% EtOH in Hex (1% TFA) | 484.2 |
| 46 | 139b or 139a | 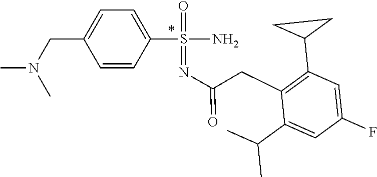 [SMILES: CC(C)c1cc(F)cc(C2CC2)c1CC(=O)N=S(N)(=O)c1ccc(CN(C)C)cc1] | (S)- or (R)- 2-(2-cyclopropyl-4-fluoro- 6-isopropylphenyl)-N- (4- ((dimethylamino)methyl) phenylsulfonimidoyl) acetamide | ChiralPak IC, 2*25 cm, 5 um | 15% EtOH in Hex | 432.1 |
| 47 | 139a or 139b | 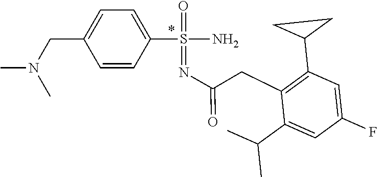 [SMILES: CC(C)c1cc(F)cc(C2CC2)c1CC(=O)N=S(N)(=O)c1ccc(CN(C)C)cc1] | (R)- or (S)- 2-(2-cyclopropyl-4-fluoro- 6-isopropylphenyl)-N- (4- ((dimethylamino)methyl) phenylsulfonimidoyl) acetamide | ChiralPak IC, 2*25 cm, 5 um | 15% EtOH in Hex | 432.1 |
| 48 | 138b or 138a | 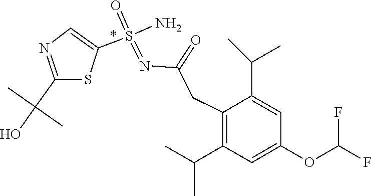 [SMILES: CC(C)c1cc(OC(F)F)cc(C(C)C)c1CC(=O)N=S(N)(=O)c1cnc(C(C)(C)O)s1] | (S)- or (R)- 2-(4-(difluoromethoxy)-2,6- diisopropylphenyl)-N- (2-(2-hydroxypropan- 2-yl)thiazol-5- ylsulfonimidoyl)acetamide | ChiralPak IG, 2*25 cm, 5 um | 8% IPA in Hex (1% TFA) | 490.1 |
| 49 | 138a or 138b | 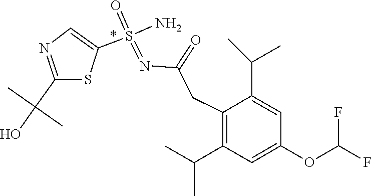 [SMILES: CC(C)c1cc(OC(F)F)cc(C(C)C)c1CC(=O)N=S(N)(=O)c1cnc(C(C)(C)O)s1] | (R)- or (S)- 2-(4-(difluoromethoxy)-2,6- diisopropylphenyl)-N- (2-(2-hydroxypropan- 2-yl)thiazol-5- ylsulfonimidoyl)acetamide | ChiralPak IG, 2*25 cm, 5 um | 8% IPA in Hex (1% TFA) | 490.1 |
| 50 | 140b or 140a | 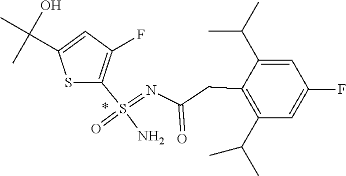 [SMILES: CC(C)c1cc(F)cc(C(C)C)c1CC(=O)N=S(N)(=O)c1sc(C(C)(C)O)cc1F] | (S)- or (R)- 2-(4-fluoro-2,6- diisopropylphenyl)- N-(3-fluoro-5- (2-hydroxypropan-2- yl)thiophen-2- ylsulfonimidoyl)acetamide | ChiralPak ID, 2*25 cm, 5 um | 30% IPA in Hex (0.1% DEA) | 459.2 |
| 51 | 140a or 140b | 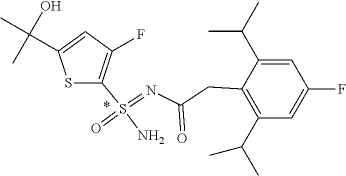 [SMILES: CC(C)c1cc(F)cc(C(C)C)c1CC(=O)N=S(N)(=O)c1sc(C(C)(C)O)cc1F] | (R)- or (S)- 2-(4-fluoro-2,6- diisopropylphenyl)- N-(3-fluoro-5- (2-hydroxypropan- 2-yl)thiophen-2- ylsulfonimidoyl)acetamide | ChiralPak ID, 2*25 cm, 5 um | 30% IPA in Hex (0.1% DEA) | 459.2 |
| 52 | 144b or 144a | 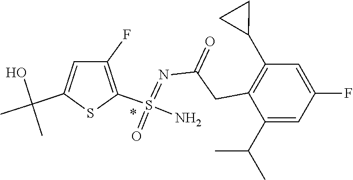 [SMILES: CC(C)c1cc(F)cc(C2CC2)c1CC(=O)N=S(N)(=O)c1sc(C(C)(C)O)cc1F] | (S)- or (R)- 2-(2-cyclopropyl-4-fluoro-6- isopropylphenyl)-N- (3-fluoro-5-(2-hydroxypropan- 2-yl)thiophen-2- ylsulfonimidoyl)acetamide | ChiralPak ID, 2*25 cm, 5 um | 30% IPA in Hex | 457.2 |
| 53 | 144a or 144b | 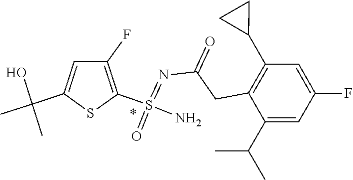 [SMILES: CC(C)c1cc(F)cc(C2CC2)c1CC(=O)N=S(N)(=O)c1sc(C(C)(C)O)cc1F] | (R)- or (S)- 2-(2-cyclopropyl-4-fluoro- 6-isopropylphenyl)-N- (3-fluoro-5-(2-hydroxypropan- 2-yl)thiophen-2- ylsulfonimidoyl)acetamide | ChiralPak ID, 2*25 cm, 5 um | 30% IPA in Hex | 457.2 |
| 54 | 145b or 145a | 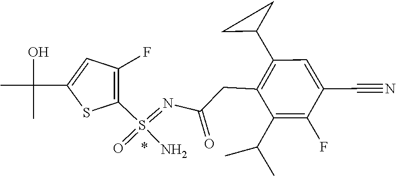 [SMILES: CC(C)c1c(F)c(C#N)cc(C2CC2)c1CC(=O)N=S(N)(=O)c1sc(C(C)(C)O)cc1F] | (S)- or (R)- 2-(4-cyano-6- cyclopropyl-3-fluoro-2- isopropylphenyl)-N-(3-fluoro- 5-(2-hydroxypropan- 2-yl)thiophen-2- ylsulfonimidoyl)acetamide | ChiralPak ID, 2*25 cm, 5 um | 30% IPA in Hex (0.1% FA) | 482.2 |
| 55 | 145a or 145b | 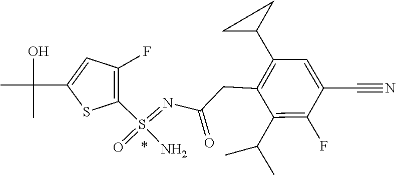 [SMILES: CC(C)c1c(F)c(C#N)cc(C2CC2)c1CC(=O)N=S(N)(=O)c1sc(C(C)(C)O)cc1F] | (R)- or (S)- 2-(4-cyano-6- cyclopropyl-3-fluoro-2- isopropylphenyl)-N-(3-fluoro- 5-(2-hydroxypropan- 2-yl)thiophen-2- ylsulfonimidoyl)acetamide | ChiralPak ID, 2*25 cm, 5 um | 30% IPA in Hex (0.1% FA) | 482.2 |
| 56 | 197b or 197a | 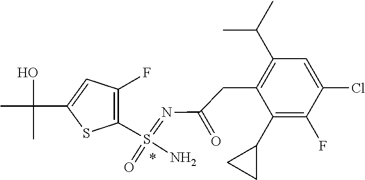 [SMILES: CC(C)c1cc(Cl)c(F)c(C2CC2)c1CC(=O)N=S(N)(=O)c1sc(C(C)(C)O)cc1F] | (S)- or (R)- 2-(4-chloro-2- cyclopropyl-3-fluoro-6- isopropylphenyl)-N-(3- fluoro-5-(2-hydroxypropan- 2-yl)thiophen-2- ylsulfonimidoyl)acetamide | ChiralPak ID, 2*25 cm, 5 um | 30% IPA in Hex (0.1% DEA) | 491.2 |
| 57 | 197a or 197b | 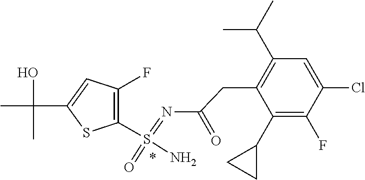 [SMILES: CC(C)c1cc(Cl)c(F)c(C2CC2)c1CC(=O)N=S(N)(=O)c1sc(C(C)(C)O)cc1F] | (R)- or (S)- 2-(4-chloro-2- cyclopropyl-3-fluoro-6- isopropylphenyl)-N-(3- fluoro-5-(2-hydroxypropan- 2-yl)thiophen-2- ylsulfonimidoyl)acetamide | ChiralPak ID, 2*25 cm, 5 um | 30% IPA in Hex (0.1% DEA) | 491.2 |
| 58 | 116 | 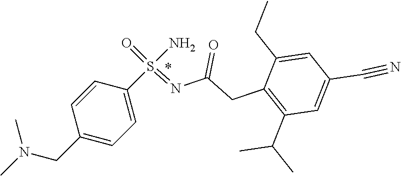 [SMILES: CCc1cc(C#N)cc(C(C)C)c1CC(=O)N=S(N)(=O)c1ccc(CN(C)C)cc1] | (S)- or (R)- 2-(4-cyano-2-ethyl-6- isopropylphenyl)-N-(4- ((dimethylamino)methyl) phenylsulfonimidoyl) acetamide | ChiralPak ADH, 2*25 cm, 5 um | 30% EtOH in Hex (0.1% DEA) | 427.2 |
| 59 | 116a or 116b | 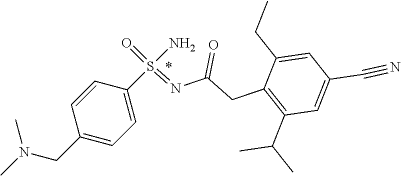 [SMILES: CCc1cc(C#N)cc(C(C)C)c1CC(=O)N=S(N)(=O)c1ccc(CN(C)C)cc1] | (R)- or (S)- 2-(4-cyano-2-ethyl-6- isopropylphenyl)-N-(4- (dimethylamino)methyl) phenylsulfonimidoyl) acetamide | ChiralPak ADH, 2*25 cm, 5 um | 30% EtOH in Hex (0.1% DEA) | 427.2 |
| 60 | 106 | 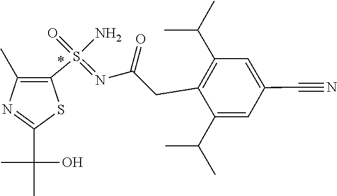 [SMILES: Cc1nc(C(C)(C)O)sc1S(N)(=O)=NC(=O)Cc1c(C(C)C)cc(C#N)cc1C(C)C] | (S)- or (R)- 2-(4-cyano-2,6- diisopropylphenyl)-N-(2-(2- hydroxypropan-2-yl)-4- methylthiazole-5- sulfonimidoyl)acetamide | ChiralPak IC, 2*25 cm, 5 um | 30% IPA in Hex (0.1% DEA) | 463.2 |
| 61 | 106a or 106b | 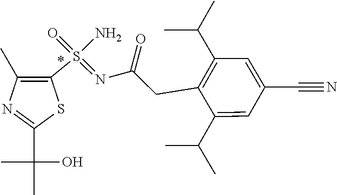 [SMILES: Cc1nc(C(C)(C)O)sc1S(N)(=O)=NC(=O)Cc1c(C(C)C)cc(C#N)cc1C(C)C] | (R)- or (S)- 2-(4-cyano-2,6- diisopropylphenyl)-N-(2-(2- hydroxypropan-2-yl)-4- methylthiazole-5- sulfonimidoyl)acetamide | ChiralPak IC, 2*25 cm, 5 um | 30% IPA in Hex (0.1% DEA) | 463.2 |
| 62 | 117a or 117b | 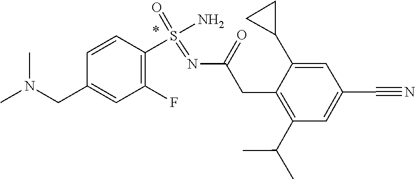 [SMILES: CC(C)c1cc(C#N)cc(C2CC2)c1CC(=O)N=S(N)(=O)c1ccc(CN(C)C)cc1F] | (S)- or (R)- 2-(4-cyano-2-cyclopropyl- 6-isopropylphenyl)-N-(4- ((dimethylamino)methyl)-2- fluorophenylsulfonimidoyl) acetamide | Chiral- CEL OD, 2*25 cm, 5 um | 30% EtOH in Hex (0.1% FA) | 457.2 |
| 63 | 117b or 117a | 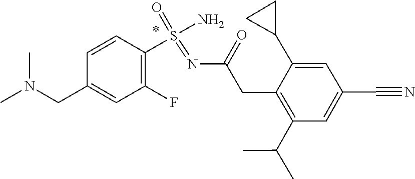 [SMILES: CC(C)c1cc(C#N)cc(C2CC2)c1CC(=O)N=S(N)(=O)c1ccc(CN(C)C)cc1F] | (R)- or (S)- 2-(4-cyano-2-cyclopropyl-6- isopropylphenyl)-N-(4- ((dimethylamino)methyl)-2- fluorophenylsulfonimidoyl) acetamide | Chiral- CEL OD, 2*25 cm, 5 um | 30% EtOH in Hex (0.1% FA) | 457.2 |
| 64 | 152b or 152a | 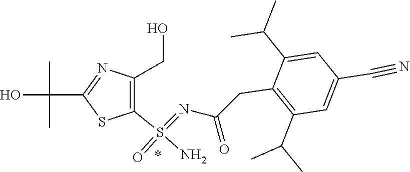 [SMILES: CC(C)c1cc(C#N)cc(C(C)C)c1CC(=O)N=S(N)(=O)c1sc(C(C)(C)O)nc1CO] | (S)- or (R)- 2-(4-cyano-2,6- diisopropylphenyl)-N-(4- (hydroxymethyl)-2-(2- hydroxypropan-2-yl)thiazol-5- ylsulfonimidoyl)acetamide | ChiralPak ID, 2*25 cm, 5 um | 80% EtOH in Hex (0.1% FA) | 480.2 |
| 65 | 152a or 152b | 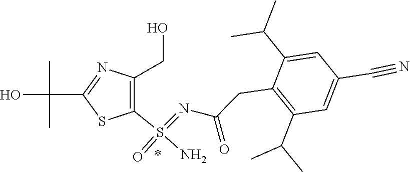 [SMILES: CC(C)c1cc(C#N)cc(C(C)C)c1CC(=O)N=S(N)(=O)c1sc(C(C)(C)O)nc1CO] | (R)- or (S)- 2-(4-cyano-2,6- diisopropylphenyl)-N-(4- (hydroxymethyl)-2-(2- hydroxypropan-2-yl)thiazol-5- ylsulfonimidoyl)acetamide | ChiralPak ID, 2*25 cm, 5 um | 80% EtOH in Hex (0.1% FA) | 480.2 |
| 66 | 150b or 150a | 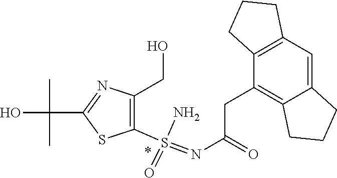 [SMILES: CC(C)(O)c1nc(CO)c(S(N)(=O)=NC(=O)Cc2c3c(cc4c2CCC4)CCC3)s1] | (S)- or (R)- 2-(1,2,3,5,6,7-hexahydros- indacen-4-yl)-N-(4- (hydroxymethyl)-2-(2- hydroxypropan-2-yl)thiazol-5- ylsulfonimidoyl)acetamide | ChiralPak ID, 2*25 cm, 5 um | 88% IPA in Hex (0.1% FA) | 450.2 |
| 67 | 150a or 150b | 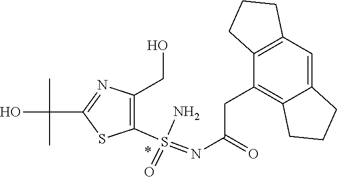 [SMILES: CC(C)(O)c1nc(CO)c(S(N)(=O)=NC(=O)Cc2c3c(cc4c2CCC4)CCC3)s1] | (R)- or (S)- 2-(1,2,3,5,6,7-hexahydros- indacen-4-yl)-N-(4- (hydroxymethyl)-2-(2- hydroxypropan-2-yl)thiazol-5- ylsulfonimidoyl)acetamide | ChiralPak ID, 2*25 cm, 5 um | 88% IPA in Hex (0.1% FA) | 450.2 |
| 68 | 148b or 148a | 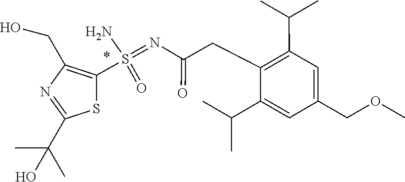 [SMILES: COCc1cc(C(C)C)c(CC(=O)N=S(N)(=O)c2sc(C(C)(C)O)nc2CO)c(C(C)C)c1] | (S)- or (R)- 2-(2,6-diisopropyl-4- (methoxymethyl)phenyl)-N-(4- (hydroxymethyl)-2-(2- hydroxypropan-2-yl)thiazol-5- ylsulfonimidoyl)acetamide | ChiralPak ID, 2*25 cm, 5 um | 75% IPA in Hex (0.1% FA) | 498.2 |
| 69 | 148a or 148b | 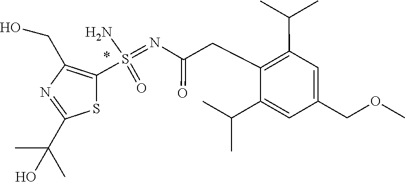 [SMILES: COCc1cc(C(C)C)c(CC(=O)N=S(N)(=O)c2sc(C(C)(C)O)nc2CO)c(C(C)C)c1] | (R)- or (S)- 2-(2,6-diisopropyl-4- (methoxymethyl)phenyl)-N-(4- (hydroxymethyl)-2-(2- hydroxypropan-2-yl)thiazol-5- ylsulfonimidoyl)acetamide | ChiralPak ID, 2*25 cm, 5 um | 75% IPA in Hex (0.1% FA) | 498.2 |
| 70 | 147b or 147a | 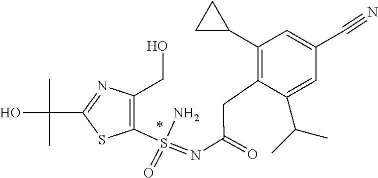 [SMILES: CC(C)c1cc(C#N)cc(C2CC2)c1CC(=O)N=S(N)(=O)c1sc(C(C)(C)O)nc1CO] | (S)- or (R)- (4-cyano-2-cyclopropyl-6- isopropylphenyl)-N-(4- (hydroxymethyl)-2-(2- hydroxypropan-2-yl)thiazol-5- ylsulfonimidoyl)acetamide | ChiralPak IC, 2*25 cm, 5 um | 15% EtOH in Hex | 477.2 |
| 71 | 147a or 147b | 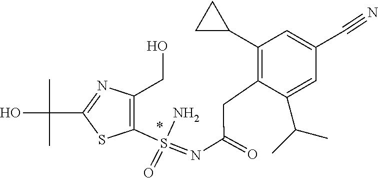 [SMILES: CC(C)c1cc(C#N)cc(C2CC2)c1CC(=O)N=S(N)(=O)c1sc(C(C)(C)O)nc1CO] | (R)- or (S)- 2-(4-cyano-2-cyclopropyl- 6-isopropylphenyl)-N-(4- (hydroxymethyi)-2-(2- hydroxypropan-2-yl)thiazol-5- ylsulfonimidoyl)acetamide | ChiralPak IC, 2*25 cm, 5 um | 15% EtOH in Hex | 477.2 |
| 72 | 114b or 114a | 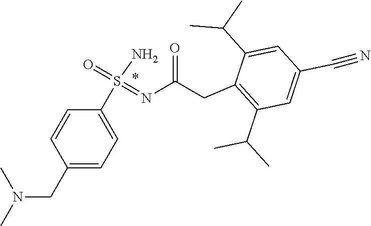 [SMILES: CC(C)c1cc(C#N)cc(C(C)C)c1CC(=O)N=S(N)(=O)c1ccc(CN(C)C)cc1] | (S)- or (R)- 2-(4-cyano-2,6- diisopropylphenyl)-N-(4- ((dimethylamino)methyl) phenylsnlfonimidoyl) acetamide | ChiralPak ID, 2*25 cm, 5 um | 50% IPA in Hex (0.1% DEA) | 441.2 |
| 73 | 114a or 114b | 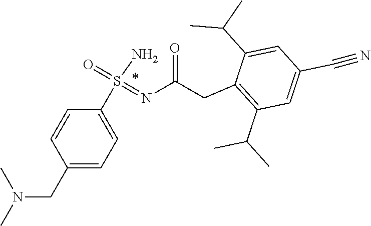 [SMILES: CC(C)c1cc(C#N)cc(C(C)C)c1CC(=O)N=S(N)(=O)c1ccc(CN(C)C)cc1] | (R)- or (S)- 2-(4-cyano-2,6- diisopropylphenyl)-N-(4- ((dimethylamino)methyl) phenylsulfonimidoyl) acetamide | ChiralPak ID, 2*25 cm, 5 um | 50% IPA in Hex (0.1% DEA) | 441.2 |
| 74 | 112b or 112a | 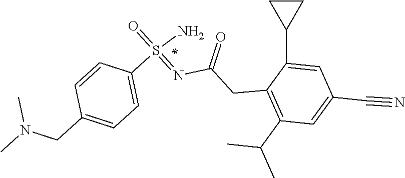 [SMILES: CC(C)c1cc(C#N)cc(C2CC2)c1CC(=O)N=S(N)(=O)c1ccc(CN(C)C)cc1] | (S)- or (R)- 2-(4-cyano-2-cyclopropyl-6- isopropylphenyl)-N-(4- ((dimethylamino)methyl) phenylsulfonimidoyl) acetamide | ChiralPak IG, 2*25 cm, 5 um | 25% IPA in Hex (0.1% DEA) | 439.2 |
| 75 | 112a or 112b | 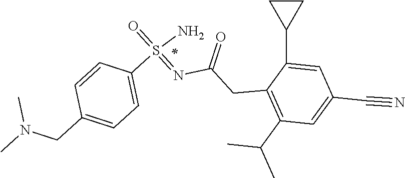 [SMILES: CC(C)c1cc(C#N)cc(C2CC2)c1CC(=O)N=S(N)(=O)c1ccc(CN(C)C)cc1] | (R)- or (S)- 2-(4-cyano-2-cyclopropyl-6- isopropylphenyl)-N-(4- ((dimethylamino)methyl) phenylsulfonimidoyl) acetamide | ChiralPak IG, 2*25 cm, 5 um | 25% EtOH in Hex (0.1% DEA) | 439.2 |
| 76 | 101b or 101a | 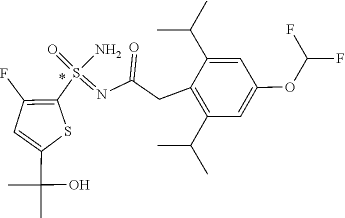 [SMILES: CC(C)c1cc(OC(F)F)cc(C(C)C)c1CC(=O)N=S(N)(=O)c1sc(C(C)(C)O)cc1F] | (S)- or (R)- 2-(4-(difluoromethoxy)-2,6- diisopropylphenyl)-N-(3- fluoro-5-(2-hydroxypropan- 2-yl)thiophen-2- ylsulfonimidoyl)acetamide | ChiralPak ID, 2*25 cm, 5 um | 30% IPA in Hex (0.1% DEA) | 507.2 |
| 77 | 101a or 101b | 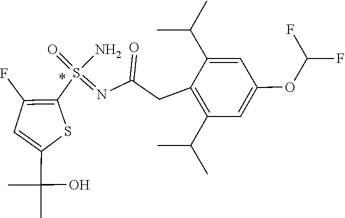 [SMILES: CC(C)c1cc(OC(F)F)cc(C(C)C)c1CC(=O)N=S(N)(=O)c1sc(C(C)(C)O)cc1F] | (R)- or (S)- (4-(difluoromethoxy)-2,6- diisopropylphenyl)-N-(3- fluoro-5-(2-hydroxypropan- 2-yl)thiophen-2- ylsulfonimidoyl)acetamide | ChiralPak ID, 2*25 cm, 5 um | 30% IPA in Hex (0.1% DEA) | 507.2 |
| 78 | 126b or 126a | 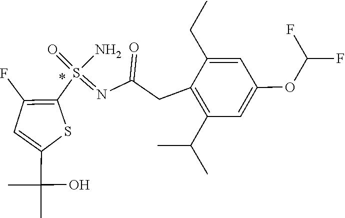 [SMILES: CCc1cc(OC(F)F)cc(C(C)C)c1CC(=O)N=S(N)(=O)c1sc(C(C)(C)O)cc1F] | (S)- or (R)- 2-(4-(difluoromethoxy)-2-ethyl- 6-isopropylphenyl)-N- (3-fluoro-5-(2-hydroxypropan- 2-yl)thiophen-2- ylsulfonimidoyl)acetamide | ChiralPak ID, 2*25 cm, 5 um | 25% IPA in Hex (0.1% DEA) | 493.2 |
| 79 | 126a or 126b | 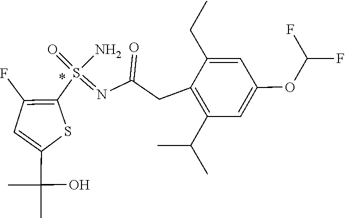 [SMILES: CCc1cc(OC(F)F)cc(C(C)C)c1CC(=O)N=S(N)(=O)c1sc(C(C)(C)O)cc1F] | (R)- or (S)- 2-(4-(difluoromethoxy)-2-ethyl- 6-isopropylphenyl)-N- (3-fluoro-5-(2-hydroxypropan- 2-yl)thiophen-2- ylsulfonimidoyl)acetamide | ChiralPak ID, 2*25 cm, 5 um | 25% IPA in Hex (0.1% DEA) | 493.2 |
| 80 | 104b or 104a | 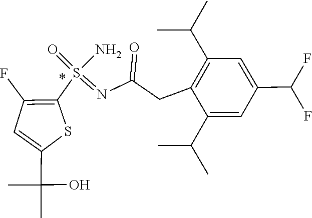 [SMILES: CC(C)c1cc(C(F)F)cc(C(C)C)c1CC(=O)N=S(N)(=O)c1sc(C(C)(C)O)cc1F] | (S)- or (R)- 2-(4-(difIuoromethyl)-2,6- diisopropylphenyl)-N-(3- fluoro-5-(2-hydroxypropan- 2-yl)thiophen-2- ylsulfonimidoyl)acetamide | ChiralPak IC, 2*25 cm, 5 um | 15% IPA in Hex (0.1% FA) | 491.2 |
| 81 | 104a or 104b | 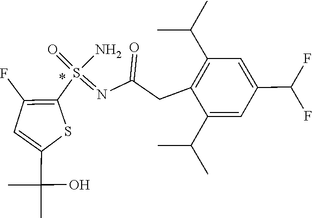 [SMILES: CC(C)c1cc(C(F)F)cc(C(C)C)c1CC(=O)N=S(N)(=O)c1sc(C(C)(C)O)cc1F] | (R)- or (S)- 2-(4-(difluoromethyl)-2,6- diisopropylphenyl)-N-(3- fluoro-5-(2-hydroxypropan- 2-yl)thiophen-2- ylsulfonimidoyl)acetamide | ChiralPak IC, 2*25 cm, 5 um | 15% IPA in Hex (0.1% FA) | 491.2 |
| 82 | 130b or 130a | 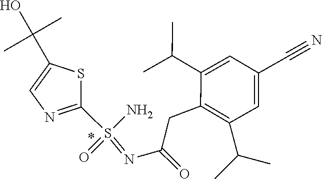 [SMILES: CC(C)c1cc(C#N)cc(C(C)C)c1CC(=O)N=S(N)(=O)c1ncc(C(C)(C)O)s1] | (R)- or (S)- 2-(4-cyano-2,6- diisopropylphenyl)-N-(5-(2- hydroxypropan-2-yl)thiazol-2- ylsulfonimidoyl)acetamide | ChiralPak IG, 2*25 cm, 5 um | 15% IPA in Hex (0.1% FA) | 449.2 |
| 83 | 130a or 130b | 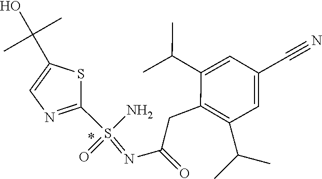 [SMILES: CC(C)c1cc(C#N)cc(C(C)C)c1CC(=O)N=S(N)(=O)c1ncc(C(C)(C)O)s1] | (S)- or (R)- 2-(4-cyano-2,6- diisopropylphenyl)-N-(5-(2- hydroxypropan-2-yl)thiazol-2- ylsulfonimidoyl)acetamide | ChiralPak IG, 2*25 cm, 5 um | 15% IPA in Hex (0.1% FA) | 449.2 |
| 84 | 107b or 107a | 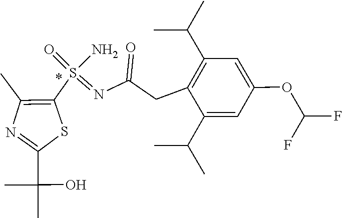 [SMILES: Cc1nc(C(C)(C)O)sc1S(N)(=O)=NC(=O)Cc1c(C(C)C)cc(OC(F)F)cc1C(C)C] | (S)- or (R)- 2-(4-(difluoromethoxy)-2,6- diisopropylphenyl)-N-(2-(2- hydroxypropan-2-yl)-4- methylthiazol-5- ylsulfonimidoyl)acetamide | ChiralPak IC, 2*25 cm, 5 um | 10% IPA in Hex (0.1% FA) | 504.2 |
| 85 | 107a or 107b | 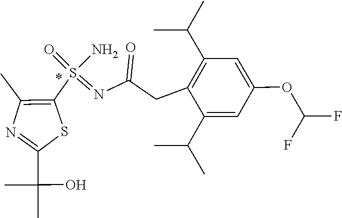 [SMILES: Cc1nc(C(C)(C)O)sc1S(N)(=O)=NC(=O)Cc1c(C(C)C)cc(OC(F)F)cc1C(C)C] | (R)- or (S)- 2-(4-(difluoromethoxy)-2,6- diisopropylphenyl)-N-(2-(2- hydroxypropan-2-yl)-4- methylthiazol-5- ylsulfonimidoyl)acetamide | ChiralPak IC, 2*25 cm, 5 um | 10% IPA in Hex (0.1% FA) | 504.2 |
| 86 | 109a or 109b | 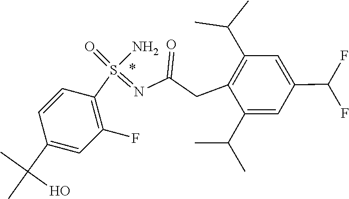 [SMILES: CC(C)c1cc(C(F)F)cc(C(C)C)c1CC(=O)N=S(N)(=O)c1ccc(C(C)(C)O)cc1F] | (S)- or (R)- 2-(4-(difluoromethyl)-2,6- diisopropylphenyl)-N-(2- fluoro-4-(2-hydroxypropan-2- yl)phenylsulfonimidoyl) acetamide | ChiralPak IF, 2*25 cm, 5 um | 15% EtOH in Hex (0.1% DEA) | 485.2 |
| 87 | 109b or 109a | 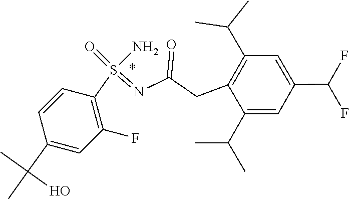 [SMILES: CC(C)c1cc(C(F)F)cc(C(C)C)c1CC(=O)N=S(N)(=O)c1ccc(C(C)(C)O)cc1F] | (R)- or (S)- 2-(4-(difluoromethyl)-2,6- diisopropylphenyl)-N-(2- fluoro-4-(2-hydroxypropan-2- yl)phenylsulfonimidoyl) acetamide | ChiralPak IF, 2*25 cm, 5 um | 15% EtOH in Hex (0.1% DEA) | 485.2 |
| 88 | 201b | 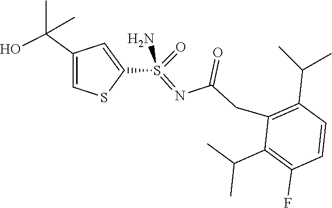 [SMILES: CC(C)c1ccc(F)c(C(C)C)c1CC(=O)N=[S@@](N)(=O)c1cc(C(C)(C)O)cs1] | (R)-N-(amino(4- (2-hydroxypropan- 2-yl)thiophen-2- yl)(oxo)-λ⁶-sulfanylidene)-2- (3-fluoro-2,6- diisopropylphenyl)acetamide | ChiralPak IF, 2*25 cm, 5 um | 15% EtOH in Hex (0.1% DEA) | 440.59 |
| 89 | 301 | 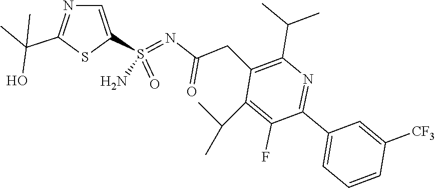 [SMILES: CC(C)c1nc(-c2cccc(C(F)(F)F)c2)c(F)c(C(C)C)c1CC(=O)N=[S@@](N)(=O)c1cnc(C(C)(C)O)s1] | (R)-N-(amino(2- (2-hydroxypropan- 2-yl)thiazol-5- yl)(oxo)-λ⁶-sulfanylidene)-2- (5-fluoro-2,4-diisopropyl-6- (3-(trifluoromethyl)phenyl) pyridin-3-yl)acetamide | ChiralPak IF, 2*25 cm, 5 um | 15% EtOH in Hex (0.1% DEA) | 586.66 |

Examples in the following table were obtained from chiral HPLC resolutions of racemic examples described above. The chiral column and eluents are listed in the table. As a convention, the faster-eluting enantiomer is always listed first in the table followed by the slower-eluting enantiomer of the pair. The symbol * at a chiral center denotes that this chiral center has been resolved and the absolute stereochemistry at that center has not been determined.

### Example 88 (Compound 241)

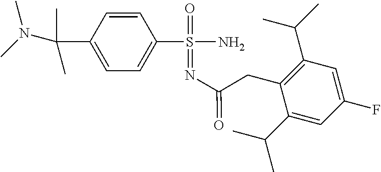 [SMILES: CC(C)c1cc(F)cc(C(C)C)c1CC(=O)N=S(N)(=O)c1ccc(C(C)(C)N(C)C)cc1]

### N-(amino(4-(2-(dimethylamino)propan-2-yl)phenyl)(oxo)-λ⁶-sulfaneylidene)-2-(4-fluoro-2,6-diisopropylphenyl)acetamide (Scheme 1A)

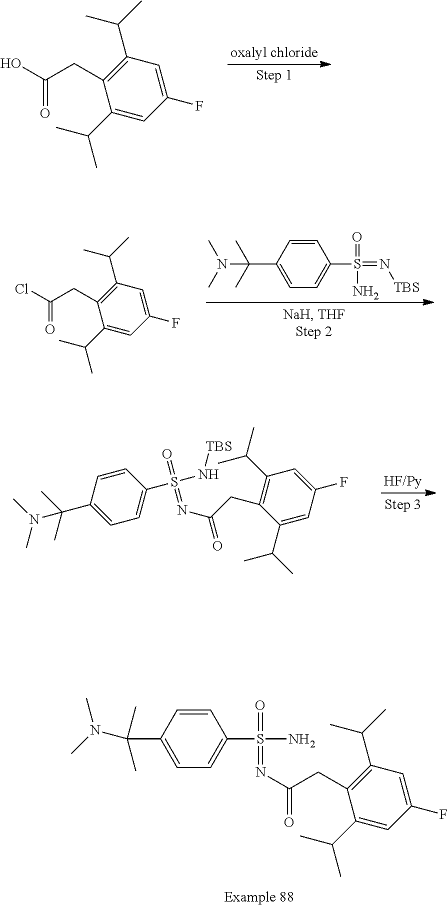

### Step 1: 2-(4-Fluoro-2,6-diisopropylphenyl)acetyl Chloride

Into a 25-mL round-bottom flask, was placed a solution of 2-[4-fluoro-2,6-bis(propan-2-yl)phenyl] acetic acid (20 mg, 0.08 mmol) in DCM (2 mL). This was followed by the addition of DMF (0.005 mL) with stirring. To this was added oxalic dichloride (0.5 mL) dropwise with stirring at 0° C. The resulting solution was stirred for 1 h at RT. The resulting mixture was concentrated. This resulted in 21 mg (97.4%) of the title compound as a yellow solid. This crude product was used in the next step.

### Step 2: N-(((tert-butyldimethylsilyl)amino)(4-(2-(dimethylamino)propan-2-yl)phenyl)(oxo)-λ⁶-sulfaneylidene)-2-(4-fluoro-2,6-diisopropylphenyl)acetamide

Into a 25-mL round-bottom flask, was placed a solution of N-(tert-butyldimethylsilyl)-4-[2-(dimethylamino) propan-2-yl]benzene-1-sulfonoimidamide (20 mg, 0.06 mmol) in THF (3 mL). To this was added NaH (60% wt. oil dispersion, 12 mg, 0.3 mmol) at 0° C. To the mixture was added a solution of 2-[4-fluoro-2,6-bis(propan-2-yl) phenyl]acetyl chloride (14.4 mg, 0.06 mmol) in DCM (1 mL) dropwise with stirring at 0° C. The resulting solution was stirred for 1 h at RT. The reaction was then quenched by the addition of 20 mL of water. The resulting solution was extracted with 2×25 mL of ethyl acetate, dried over anhydrous sodium sulfate, and concentrated. The residue was eluted from silica gel with ethyl acetate/petroleum ether (1:1). This resulted in 20 mg (61.7%) of the title compound as a white solid. MS-ESI: 576 (M+1).

### Step 3: N-(amino(4-(2-(dimethylamino)propan-2-yl)phenyl)(oxo)-λ⁶-sulfaneylidene)-2-(4-fluoro-2,6-diisopropylphenyl)acetamide

Into a 25-mL round-bottom flask, was placed a solution of N-[[(tert-butyldimethylsilyl)amino]([4-[2-(dimethylamino)propan-2-yl]phenyl])oxo-λ⁶-sulfanylidene]-2-[4-fluoro-2,6-bis(propan-2-yl)phenyl]acetamide (20 mg, 0.03 mmol) in DCM (2 mL). This was followed by the addition of HF-Pyridine (70% wt., 1 mL) dropwise with stirring. The resulting solution was stirred for 30 min at RT. The resulting mixture was washed with 20 mL of H₂O. The resulting solution was extracted with 2×25 mL of ethyl acetate dried over anhydrous sodium sulfate. The resulting mixture was concentrated. The crude product was purified by Prep-HPLC with the following conditions: Column, XBridge Shield RP18 OBD, 19*250 mm, 10 um; mobile phase, water (10 mM NH₄HCO₃) and ACN (30% to 50% ACN gradient in 10 min); Detector, UV220/254 nm. This resulted in 5.2 mg (32.4%) of Example 88 as a white solid. MS-ESI: 462.3 (M+1). ¹H-NMR (400 MHz, CD₃OD-d₄) δ 7.87 (d, J=8.4 Hz, 2H), 7.69 (d, J=8.4 Hz, 2H), 6.76 (d, J=10.4 Hz, 2H), 3.74 (s, 2H), 3.12-3.08 (m, 2H), 2.16 (s, 6H), 1.39 (s, 6H), 1.11 (dd, J=15.2, 6.8 Hz, 12H).

TABLE 18

| | | | | Exact |
|---|---|---|---|---|
| | | | | Mass |
| Ex. # | Cmpd # | Structure | IUPAC Name | [M + H]⁺ |
| 89 | 219 | 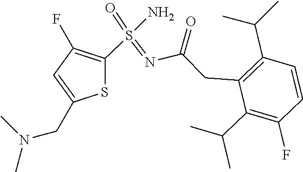 [SMILES: CC(C)c1ccc(F)c(C(C)C)c1CC(=O)N=S(N)(=O)c1sc(CN(C)C)cc1F] | N-(amino(5-((dimethylamino) methyl)-3-fluorothiophen-2- yl)(oxo)-λ⁶-sulfaneylidene)- 2-(3-fluoro-2,6- diisopropylphenyl)acetamide | 458 |
| 90 | 218 | 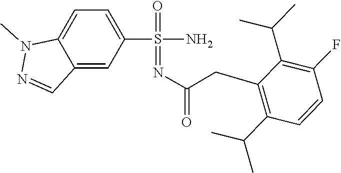 [SMILES: CC(C)c1ccc(F)c(C(C)C)c1CC(=O)N=S(N)(=O)c1ccc2c(cnn2C)c1] | N-(amino(1-methyl-1H-indazol- 5-yl)(oxo)-λ⁶-sulfaneylidene)-2- (3-fluoro-2,6-diisopropylphenyl)acetamide | 431 |
| 91 | 217 | 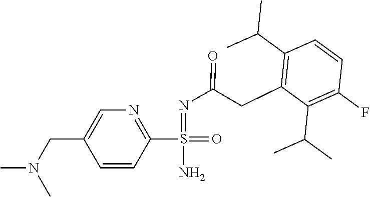 [SMILES: CC(C)c1ccc(F)c(C(C)C)c1CC(=O)N=S(N)(=O)c1ccc(CN(C)C)cn1] | N-(amino(5-((dimethylamino) methyl)pyridin-2-yl)(oxo)-λ⁶- sulfaneylidene)-2-(3-fluoro-2,6- diisopropylphenyl)acetamide | 435 |
| 92 | 214 | 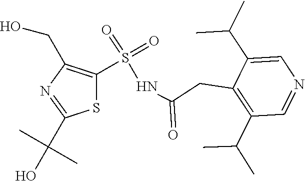 [SMILES: CC(C)c1cncc(C(C)C)c1CC(=O)NS(=O)(=O)c1sc(C(C)(C)O)nc1CO] | 2-(3,5-diisopropylpyridin-4-yl)- N-((4-(hydroxymethyl)-2-(2- hydroxypropan-2-yl)thiazol- 5-yl)sulfonyl)acetamide | 456 |

Examples in the following table were prepared using similar conditions as described in Example 88 and Scheme 1A from appropriate starting materials.

### Example 93 (Compound 235)

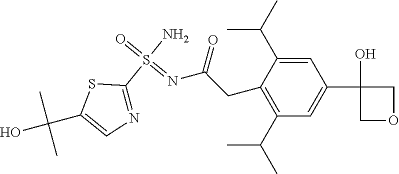 [SMILES: CC(C)c1cc(C2(O)COC2)cc(C(C)C)c1CC(=O)N=S(N)(=O)c1ncc(C(C)(C)O)s1]

### N-(amino(5-(2-hydroxypropan-2-yl)thiazol-2-yl)(oxo)-λ⁶-sulfaneylidene)-2-(4-(3-hydroxyoxetan-3-yl)-2,6-diisopropylphenyl)acetamide (Scheme 1B)

### Example 94 (Compound 230)

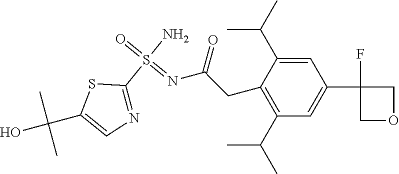 [SMILES: CC(C)c1cc(C2(F)COC2)cc(C(C)C)c1CC(=O)N=S(N)(=O)c1ncc(C(C)(C)O)s1]

### N-(amino(5-(2-hydroxypropan-2-yl)thiazol-2-yl)(oxo)-λ⁶-sulfaneylidene)-2-(4-(3-fluorooxetan-3-yl)-2,6-diisopropylphenyl)acetamide (Scheme 1B)

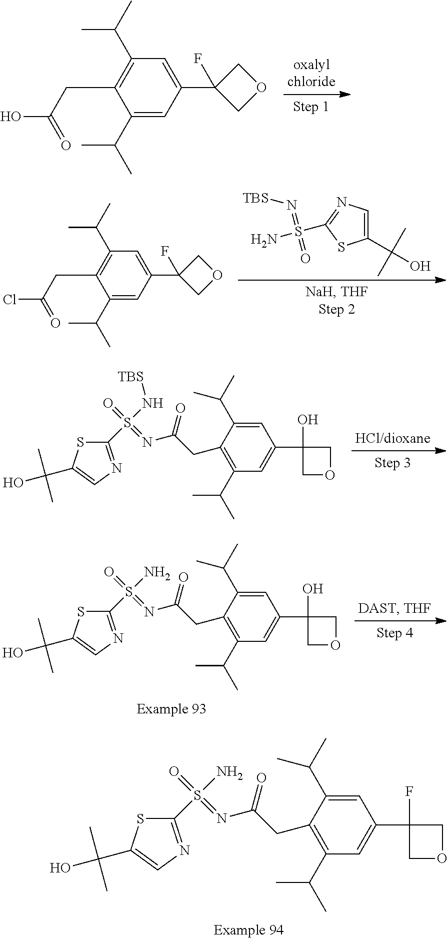

### Step 1: 2-(4-(3-fluorooxetan-3-yl)-2,6-diisopropylphenyl)acetyl Chloride

Into a 50-mL round-bottom flask, was placed 2-[4-(3-fluorooxetan-3-yl)-2,6-bis(propan-2-yl)phenyl] acetic acid (150 mg, 0.51 mmol) in DCM (3 mL) and DMF (0.05 mL). This was followed by the addition of oxalic dichloride (0.5 mL) dropwise with stirring at RT. The resulting solution was stirred for 30 min at RT. The resulting mixture was concentrated under vacuum. This resulted in 159 mg (99.7%) of the title compound as a light yellow solid.

### Step 2: N-(((tert-butyldimethylsilyl)amino)(5-(2-hydroxypropan-2-yl)thiazol-2-yl)(oxo)-λ⁶-sulfaneylidene)-2-(4-(3-hydroxyoxetan-3-yl)-2,6-diisopropylphenyl)acetamide

Into a 50-mL round-bottom flask, was placed N-(tert-butyldimethylsilyl)-5-(2-hydroxypropan-2-yl)-1,3-thiazole-2-sulfonoimidamide (173 mg, 0.52 mmol) in THF (5 mL). This was followed by the addition of NaH (60% wt. oil dispersion, 62.4 mg, 1.56 mmol) in portions at 0° C. The resulting solution was stirred for 10 min at RT. Then to the above was added a solution of 2-[4-(3-fluorooxetan-3-yl)-2,6-bis(propan-2-yl)phenyl]acetyl chloride (159 mg, 0.51 mmol) in THF (2 mL) dropwise with stirring at 0° C. The resulting solution was stirred for 2 h at RT. The reaction was then quenched by the addition of 5 mL of water. The resulting solution was extracted with 3×5 mL of ethyl acetate. The organic layers were combined, dried over anhydrous sodium sulfate, and concentrated under vacuum. This resulted in 200 mg (64.5%) of the title compound as an off-white solid. MS-ESI. 610 (M+1).

### Step 3: N-(amino(5-(2-hydroxypropan-2-yl)thiazol-2-yl)(oxo)-λ⁶-sulfaneylidene)-2-(4-(3-hydroxyoxetan-3-yl)-2,6-diisopropylphenyl)acetamide

Into a 50-mL round-bottom flask, was placed N-[[(tert-butyldimethylsilyl)amino][5-(2-hydroxypropan-2-yl)-1,3-thiazol-2-yl]oxo-λ⁶-sulfanylidene]-2-[4-(3-hydroxyoxetan-3-yl)-2,6-bis(propan-2-yl)phenyl]acetamide (200 mg, 0.33 mmol) in THF (2 mL), to the stirred solution was added HCl/dioxane (4 M, 5 mL). The resulting solution was stirred overnight at RT. The resulting mixture was concentrated under vacuum. The crude product was purified by Prep-HPLC with the following conditions: Column, XBridge Prep C18 OBD, 5 um, 19*150 mm; mobile phase, water (10 mM NH₄HCO₃) and ACN (5% to 40% ACN gradient over 8 min); Detector, UV 254/210 nm. This resulted in 14.0 mg (8.61%) of Example 93 as a white solid. MS-ESI: 496.3 (M+1). ¹H-NMR: (DMSO-d₆, 400 MHz): δ 7.40 (s, 1H), 7.23 (s, 2H), 6.14 (s, 1H), 5.54 (s, 1H), 4.73-4.66 (m, 4H), 3.92 (s, 1H), 3.49-3.46 (m, 2H), 3.31-3.22 (m, 2H), 1.47 (s, 6H), 1.12 (d, J=8.4 Hz, 6H), 1.10 (d, J=8.4 Hz, 6H).

### Step 4: N-(amino(5-(2-hydroxypropan-2-yl)thiazol-2-yl)(oxo)-λ⁶-sulfaneylidene)-2-(4-(3-fluorooxetan-3-yl)-2,6-diisopropylphenyl)acetamide

Into a 50-mL 3-necked round-bottom flask purged and maintained with an inert atmosphere of nitrogen, was placed N-[amino[5-(2-hydroxypropan-2-yl)-1,3-thiazol-2-yl]oxo-λ⁶-sulfanylidene]-2-[4-(3-hydroxyoxetan-3-yl)-2,6-bis(propan-2-yl)phenyl]acetamide (83 mg, 0.17 mmol) in THF (5 mL). This was followed by the addition of a solution of DAST (54.0 mg, 0.33 mmol) in DCM (1 mL) dropwise with stirring at 0° C. The resulting solution was stirred overnight at RT. The resulting mixture was concentrated under vacuum. The crude product was purified by Prep-HPLC with the following conditions: Column, XBridge BEH130 Prep C18 OBD, 19×150 mm, 5 um 13 nm; mobile phase, water (10 mM NH₄HCO₃) and ACN (30% to 60% ACN gradient in 7 min); Detector, UV 254/210 nm. This resulted in 15.1 mg (17.9%) of Example 94 as a white solid. MS-ESI: 498.2 (M+1). ¹H NMR (DMSO-d₆, 400 MHz): δ 7.96 (s, 2H), 7.76 (s, 1H), 7.18 (s, 2H), 5.87 (s, 1H), 4.98-4.87 (m, 4H), 3.77-3.65 (m, 2H), 3.13-3.05 (m, 2H), 1.51 (s, 6H), 1.13 (d, J=8.4 Hz, 6H), 1.11 (d, J=8.4 Hz, 6H).

TABLE 19

| | | | | Exact |
|---|---|---|---|---|
| | Cmpd | | | Mass |
| Ex. # | # | Structure | IUPAC Name | [M + H]⁺ |
| 95 | 245 | 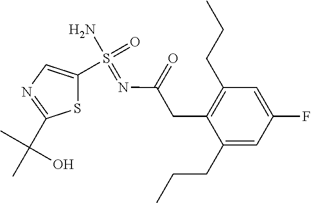 [SMILES: CCCc1cc(F)cc(CCC)c1CC(=O)N=S(N)(=O)c1cnc(C(C)(C)O)s1] | N-(amino(2-(2-hydroxypropan- 2-yl)thiazol-5-yl(oxo)-λ⁶- sulfaneylidene)-2-(4-fluoro-2,6- dipropylphenyl)acetamide | 442 |

Examples in the following table were prepared using similar conditions as described in Example 94 and Scheme 1B from appropriate starting materials.

### Example 96 (Compound 244)

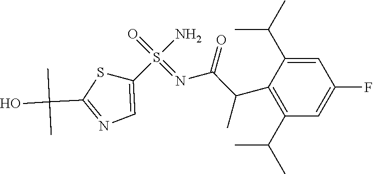 [SMILES: CC(C)c1cc(F)cc(C(C)C)c1C(C)C(=O)N=S(N)(=O)c1cnc(C(C)(C)O)s1]

### N-(amino(2-(2-hydroxypropan-2-yl)thiazol-5-yl)(oxo)-λ⁶-sulfaneylidene)-2-(4-fluoro-2,6-diisopropylphenyl)propanamide (Scheme 1C)

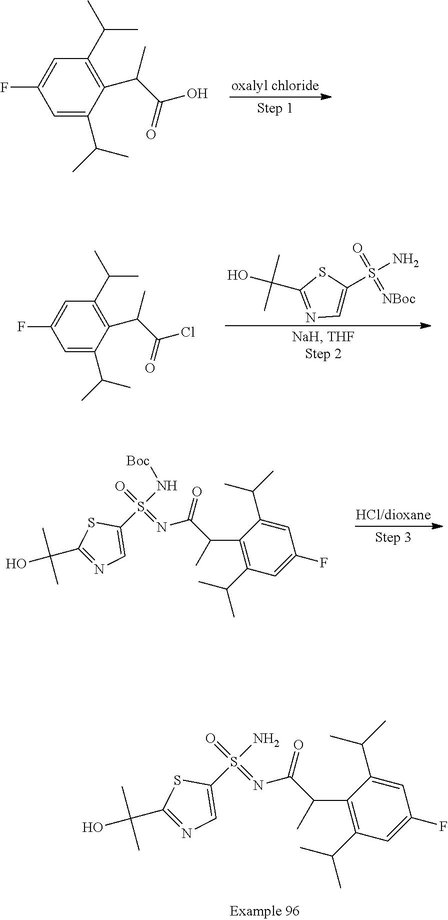

### Step 1: 2-(4-Fluoro-2,6-diisopropylphenyl)propanoyl Chloride

Into a 50-mL round-bottom flask, was placed 2-[4-fluoro-2,6-bis(propan-2-yl)phenyl]propanoic acid (200 mg, 0.79 mmol) in DCM (20 mL), this was followed by the addition of oxalic dichloride (2 mL). The resulting solution was stirred for 15 min at RT. The resulting mixture was concentrated. This resulted in 200 mg (93.1%) of the title compound as off-white oil.

### Step 2: Tert-butyl(N-(2-(4-fluoro-2,6-diisopropylphenyl)propanoyl)-2-(2-hydroxypropan-2-yl) thiazole-5-sulfonimidoyl)carbamate

Into a 50-mL round-bottom flask, was placed tert-butyl N-[amino[2-(2-hydroxypropan-2-yl)-1,3-thiazol-5-yl] methylidene-λ⁶-sulfanylidene]carbamate (235.9 mg, 0.74 mmol) in THF (20 mL). To the mixture was added 2-[4-fluoro-2,6-bis(propan-2-yl)phenyl]propanoyl chloride (200 mg, 0.74 mmol) and NaH (60% wt. oil dispersion, 59.2 mg, 1.48 mmol). The resulting solution was stirred for 16 h at RT. The reaction was then quenched by the addition of 20 mL of water. The resulting solution was extracted with 3×20 ml of ethyl acetate, dried over anhydrous sodium sulfate, and concentrated. The resulting mixture was concentrated. The residue was eluted from silica gel with ethyl acetate/petroleum ether (1:1). This resulted in 200 mg (48.9%) of the title compound as an off-white solid. MS-ESI: 556 (M+1).

### Step 3: N-(amino(2-(2-hydroxypropan-2-yl)thiazol-5-yl)(oxo)-λ⁶-sulfaneylidene)-2-(4-fluoro-2,6-diisopropylphenyl)propanamide

Into a 50-mL round-bottom flask, was placed tert-butyl N-[([2-[4-fluoro-2,6-bis(propan-2-yl)phenyl]propanoyl]imino)[2-(2-hydroxypropan-2-yl)-1,3-thiazol-5-yl]methylidene-λ⁶-sulfanyl]carbamate (100 mg, 0.18 mmol) in THF (5 mL). To the stirred solution was added HCl/dioxane (10 mL, 4 M) dropwise. The resulting solution was stirred for 16 h at RT. The resulting mixture was concentrated. The crude product was purified by Prep-HPLC with the following conditions: Column: XBridge Shield RP18 OBD Column 19*250 mm, 10 um; Mobile Phase A: water (10 mM NH₄HCO₃), Mobile Phase B: ACN; Flow rate: 25 mL/min; Gradient: 27% B to 45% B in 9 min; 254/210 nm; Rt: 7.77 min. This resulted in 20 mg (24.4%) of Example 96 as a white solid. MS-ESI: 456.2 (M+1). ¹H-NMR (400 MHz, DMSO-d₆, ppm) δ: 8.00-7.92 (m, 3H), 7.05-6.91 (m, 2H), 6.25 (s, 1H), 3.87-3.81 (m, 1H), 3.16-2.98 (m, 2H), 1.49 (s, 6H), 1.27-1.24 (m, 3H), 1.19-1.13 (m, 12H).

TABLE 20

| | | | | Exact |
|---|---|---|---|---|
| Ex. | Cmpd | | | Mass |
| # | # | Structure | IUPAC Name | [M + H]⁺ |
| 97 | 221 | 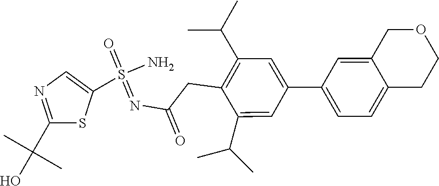 [SMILES: CC(C)c1cc(-c2ccc3c(c2)COCC3)cc(C(C)C)c1CC(=O)N=S(N)(=O)c1cnc(C(C)(C)O)s1] | N-(amino(2-(2-hydroxypropan-2- yl)thiazol-5-yl)(oxo)-λ⁶- sulfaneylidene)-2-(4- (isochroman-7-yl)-2,6- diisopropylphenyl)acetamide | 556 |
| 98 | 220 | 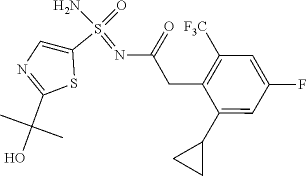 [SMILES: CC(C)(O)c1ncc(S(N)(=O)=NC(=O)Cc2c(C3CC3)cc(F)cc2C(F)(F)F)s1] | N-(amino(2-(2-hydroxypropan- 2-yl)thiazol-5-yl)(oxo)-λ⁶- sulfaneylidene)-2-(2- cyclopropyl-4-fluoro-6- (trifluoromethyl)phenyl)acetamide | 466 |
| 99 | 304 | 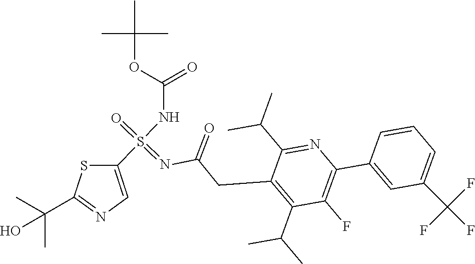 [SMILES: CC(C)c1nc(-c2cccc(C(F)(F)F)c2)c(F)c(C(C)C)c1CC(=O)N=S(=O)(NC(=O)OC(C)(C)C)c1cnc(C(C)(C)O)s1] | tert-butyl (N-(2-(5-fluoro- 2,4-diisopropyl-6-(3- (trifluoromethyl)phenyl) pyridin-3-yl)acetyl)-2-(2- hydroxypropan-2-yl)thiazole-5- sulfonimidoyl)carbamate | 687 |
| 100 | 301 | 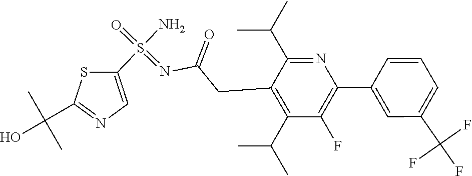 [SMILES: CC(C)c1nc(-c2cccc(C(F)(F)F)c2)c(F)c(C(C)C)c1CC(=O)N=S(N)(=O)c1cnc(C(C)(C)O)s1] | N-(amino(2-(2-hydroxypropan-2- yl)thiazol-5-yl)(oxo)-λ⁶- sulfaneylidene)-2-(5-fluoro- 2,4-diisopropyl-6-(3- (trifluoromethyl)phenyl) pyridin-3-yl)acetamide | 587 |
| 101 | 212 | 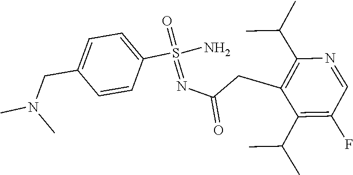 [SMILES: CC(C)c1ncc(F)c(C(C)C)c1CC(=O)N=S(N)(=O)c1ccc(CN(C)C)cc1] | N-(amino(4-((dimethylamino) methyl)phenyl)(oxo)-λ⁶- sulfaneylidene)-2-(5-fluoro- 2,4-diisopropylpyridin-3- yl)acetamide | 435 |

Examples in the following table were prepared using similar conditions as described in Example 96 and Scheme 1C from appropriate starting materials.

### Example 102 (Compound 240)

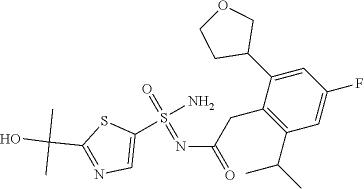 [SMILES: CC(C)c1cc(F)cc(C2CCOC2)c1CC(=O)N=S(N)(=O)c1cnc(C(C)(C)O)s1]

### N-(amino(2-(2-hydroxypropan-2-yl)thiazol-5-yl)(oxo)-λ⁶-sulfaneylidene)-2-(4-fluoro-2-isopropyl-6-(tetrahydrofuran-3-yl)phenyl)acetamide (Scheme 1C)

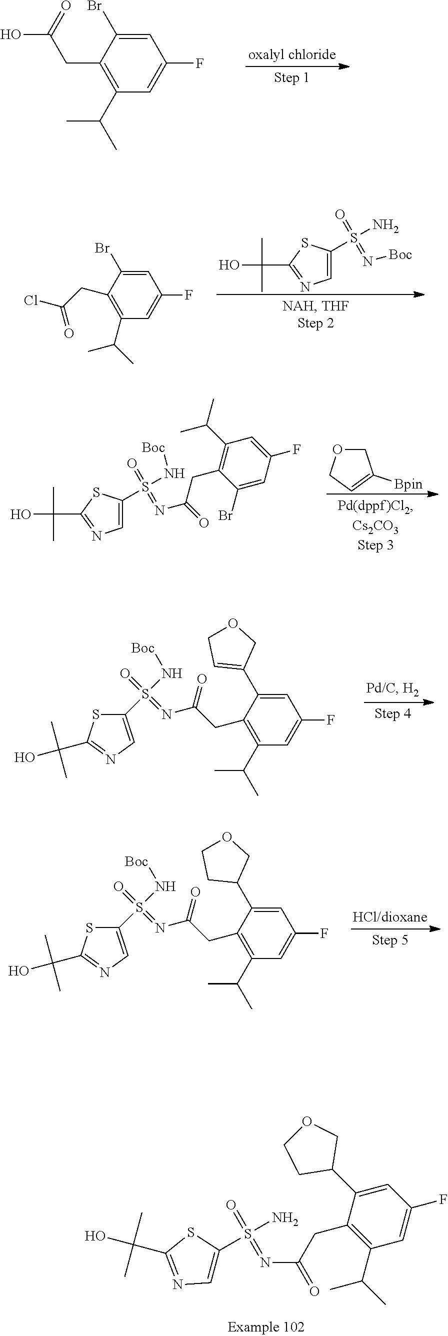

### Step 1: 2-(2-Bromo-4-fluoro-6-isopropylphenyl)acetyl Chloride

Into a 100-mL round-bottom flask, was placed 2-[2-bromo-4-fluoro-6-(propan-2-yl)phenyl]acetic acid (100 mg, 0.36 mmol) in DCM (25 mL) and DMF (0.01 mL). To the above solution was added oxalic dichloride (0.5 mL) dropwise. The resulting solution was stirred for 30 min at RT. The resulting mixture was concentrated. This resulted in 100 mg (93.7%) of the title compound as yellow oil.

### Step 2: Tert-butyl (N-(2-(2-bromo-4-fluoro-6-isopropylphenyl)acetyl)-2-(2-hydroxypropan-2-yl) thiazole-5-sulfonimidoyl)carbamate

Into a 100-mL round-bottom flask, was placed tert-butyl N-[amino[2-(2-hydroxypropan-2-yl)-1,3-thiazol-5-yl]oxo-λ⁶-sulfanylidene]carbamate (116.7 mg, 0.36 mmol) in THF (25 mL). To the mixture was added NaH (60% wt. oil dispersion, 29.2 mg, 0.73 mmol) in portions with stirring. The resulting solution was stirred for 30 min at RT. 2-[2-bromo-4-fluoro-6-(propan-2-yl)phenyl]acetyl chloride (100 mg, 0.34 mmol) was added to the solution. The resulting solution was stirred for an additional 1 h at RT. The reaction was then quenched by the addition of 5 mL of water. The resulting solution was extracted with 2×10 ml of ethyl acetate and concentrated. This resulted in 219 mg (crude) title compound as a white solid. MS-ESI. 578 (M+1).

### Step 3: Tert-butyl (N-(2-(2-(2, 5-dihydrofuran-3-yl)-4-fluoro-6-isopropylphenyl)acetyl)-2-(2-hydroxypropan-2-yl)thiazole-5-sulfonimidoyl)carbamate

Into a 100-mL round-bottom flask purged and maintained with an inert atmosphere of nitrogen, was placed tert-butyl (N-(2-(2-bromo-4-fluoro-6-isopropylphenyl)acetyl)-2-(2-hydroxypropan-2-yl) thiazole-5-sulfonimidoyl)carbamate (395 mg, 0.68 mmol) in dioxane (60 mL) and H₂O (12 mL). This was followed by the addition of Cs₂CO₃ (667.4 mg, 2.05 mmol), 2-(2, 5-dihydrofuran-3-yl)-4,4, 5, 5-tetramethyl-1,3,2-dioxaborolane (201 mg, 1.02 mmol) and Pd(dppf)Cl₂ (50 mg, 0.07 mmol). The resulting solution was stirred for 16 h at 95° C. in an oil bath. The resulting solution was diluted with H₂O (50 mL), extracted with 2×15 ml of ethyl acetate and concentrated. The residue was eluted from silica gel with ethyl acetate/petroleum ether (1:1). This resulted in 187 mg (50.9%) of the title compound as a dark orange solid. MS-ESI: 568 (M+1).

### Step 4: Tert-butyl (N-(2-(4-fluoro-2-isopropyl-6-(tetrahydrofuran-3-yl)phenyl)acetyl)-2-(2-hydroxypropan-2-yl)thiazole-5-sulfonimidoyl)carbamate

Into a 100-mL round-bottom flask, was placed tert-butyl N-([2-[2-(2, 5-dihydrofuran-3-yl)-4-fluoro-6-(propan-2-yl)phenyl]acetamido][2-(2-hydroxypropan-2-yl)-1,3-oxazol-5-yl]oxo-λ⁶-sulfanylidene)carbamate (237 mg, 0.43 mmol) in methanol (25 mL). To the above solution was added Pd/C (10% wt., 30 mg) with stirring. The solution was evacuated and filled three times with hydrogen. The resulting solution was stirred for 16 h at RT. The solids were filtered out. The resulting mixture was concentrated. The residue was eluted from silica gel with DCM/methanol (10:1). This resulted in 234 mg (98.3%) of the title compound as a white solid. MS-ESI: 570 (M+1).

### Step 5: N-(amino(2-(2-hydroxypropan-2-yl)thiazol-5-yl)(oxo)-λ⁶-sulfaneylidene)-2-(4-fluoro-2-isopropyl-6-(tetrahydrofuran-3-yl)phenyl)acetamide

Into a 50-mL round-bottom flask, was placed tert-butyl N-[([2-[4-fluoro-2-(oxolan-3-yl)-6-(propan-2-yl)phenyl]acetyl]imino)[2-(2-hydroxypropan-2-yl)-1,3-thiazol-5-yl]oxo-λ⁶-sulfanyl]carbamate (200 mg, 0.35 mmol) in HCl/dioxane (4M, 10 mL). The resulting solution was stirred for 1 h at RT. The resulting mixture was concentrated. The crude product was purified by Prep-HPLC with the following conditions: Column, XBridge Prep C18 OBD, 5 um, 19*150 mm; mobile phase, water (10 mM NH₄HCO₃) and ACN (7% to 45% ACN gradient in 7 min); Detector, UV 254/210 nm. This resulted in 20 mg (12.13%) of Example 102 as a white solid. MS-ESI: 470 (M+1).

¹H-NMR (400 MHz, DMSO-d₆, ppm) δ: 8.06 (br s, 2H), 7.03 (d, J=8.0 Hz, 1H), 7.03 (d, J=12.4 Hz, 1H), 6.28 (s, 1H), 4.01-3.97 (m, 1H), 3.91-3.89 (m, 1H), 3.79-3.77 (m, 1H), 3.55 (s, 2H), 3.55-3.45 (m, 3H), 2.91-2.86 (m, 1H), 2.25-2.23 (m, 1H), 1.48 (s, 6H), 1.06 (d, J=6.8 Hz, 3H), 1.01 (d, J=6.8 Hz, 3H).

TABLE 21

| | | | | Exact |
|---|---|---|---|---|
| Ex. | Cmpd | | | Mass |
| # | # | Structure | IUPAC Name | [M + H]⁺ |
| 103 | 258 | 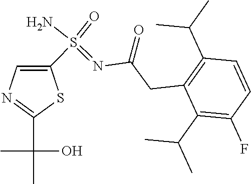 [SMILES: CC(C)c1ccc(F)c(C(C)C)c1CC(=O)N=S(N)(=O)c1cnc(C(C)(C)O)s1] | N-(amino(2-(2-hydroxypropan- 2-yl)thiazol-5-yl)(oxo)- λ⁶-sulfaneylidene)-2-(3-fluoro-2,6- diisopropylphenyl)acetamide | 442 |
| 104 | 257 | 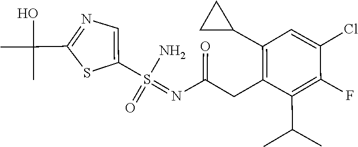 [SMILES: CC(C)c1c(F)c(Cl)cc(C2CC2)c1CC(=O)N=S(N)(=O)c1cnc(C(C)(C)O)s1] | N-(amino(2-(2-hydroxypropan- 2-yl)thiazol-5-yl)(oxo)- λ⁶-sulfaneylidene)-2-(4-chloro- 6-cyclopropyl-3-fluoro-2- isopropylphenyl)acetamide | 474 |
| 105 | 256 | 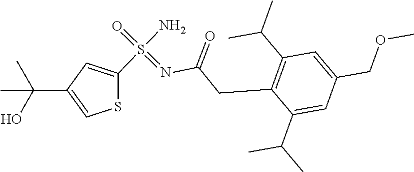 [SMILES: COCc1cc(C(C)C)c(CC(=O)N=S(N)(=O)c2cc(C(C)(C)O)cs2)c(C(C)C)c1] | N-(amino(4-(2-hydroxypropan- 2-yl)thiophen-2-yl)(oxo)- λ⁶-sulfaneylidene)-2- (2,6-diisopropyl-4- (methoxymethyl)phenyl) acetamide | 467 |
| 106 | 201 | 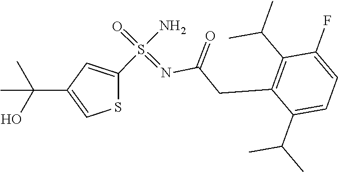 [SMILES: CC(C)c1ccc(F)c(C(C)C)c1CC(=O)N=S(N)(=O)c1cc(C(C)(C)O)cs1] | N-(ainino(4-(2-hydroxypropan- 2-yl)thiophen-2-yl)(oxo)- λ⁶-sulfaneylidene)-2-(3-fluoro-2,6- diisopropylphenyl)acetamide | 441 |
| 107 | 255 | 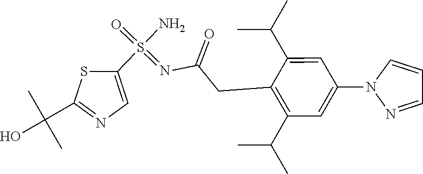 [SMILES: CC(C)c1cc(-n2cccn2)cc(C(C)C)c1CC(=O)N=S(N)(=O)c1cnc(C(C)(C)O)s1] | N-(amino(2-(2-hydroxypropan- 2-yl)thiazol-5-yl)(oxo)- λ⁶-sulfaneylidene)-2-(2,6- diisopropyl-4-(1H-pyrazol-1- yl)phenyl)acetamide | 490 |
| 108 | 254 | 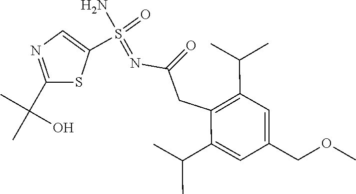 [SMILES: COCc1cc(C(C)C)c(CC(=O)N=S(N)(=O)c2cnc(C(C)(C)O)s2)c(C(C)C)c1] | N-(amino(2-(2-hydroxypropan- 2-yl)thiazol-5-yl)(oxo)- λ⁶-sulfaneylidene)-2- (2,6-diisopropyl-4- (methoxymethyl)phenyl) acetamide | 468 |
| 109 | 253 | 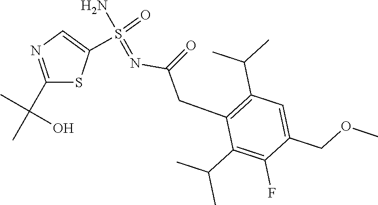 [SMILES: COCc1cc(C(C)C)c(CC(=O)N=S(N)(=O)c2cnc(C(C)(C)O)s2)c(C(C)C)c1F] | N-(amino(2-(2-hydroxypropan- 2-yl)thiazol-5-yl)(oxo)- λ⁶-sulfaneylidene)-2-(3- fluoro-2,6-diisopropyl-4- (methoxymethyl)phenyl)acetamide | 486 |
| 110 | 252 | 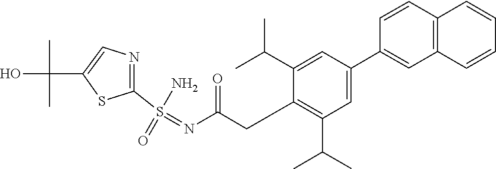 [SMILES: CC(C)c1cc(-c2ccc3ccccc3c2)cc(C(C)C)c1CC(=O)N=S(N)(=O)c1ncc(C(C)(C)O)s1] | N-(amino(5-(2-hydroxypropan- 2-yl)thiazol-2-yl)(oxo)- λ⁶-sulfaneylidene)-2-(2,6- diisopropyl-4-(naphthalen-2- yl)phenyl)acetamide | 550 |
| 111 | 251 | 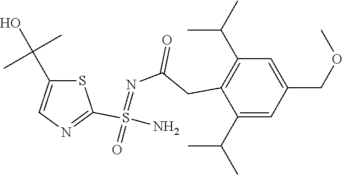 [SMILES: COCc1cc(C(C)C)c(CC(=O)N=S(N)(=O)c2ncc(C(C)(C)O)s2)c(C(C)C)c1] | N-(amino(5-(2-hydroxypropan- 2-yl)thiazol-2-yl)(oxo)- λ⁶-sulfaneylidene)-2- (2,6-diisopropyl-4- (methoxymethyl)phenyl) acetamide | 468 |
| 112 | 250 | 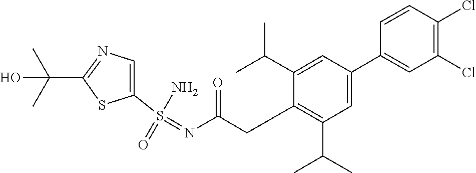 [SMILES: CC(C)c1cc(-c2ccc(Cl)c(Cl)c2)cc(C(C)C)c1CC(=O)N=S(N)(=O)c1cnc(C(C)(C)O)s1] | N-(amino(2-(2-hydroxypropan- 2-yl)thiazol-5-yl)(oxo)- λ⁶-sulfaneylidene)-2-(3′,4′- dichloro-3,5-diisopropyl- [1,1′-biphenyl]-4-yl)acetamide | 568 |
| 113 | 249 | 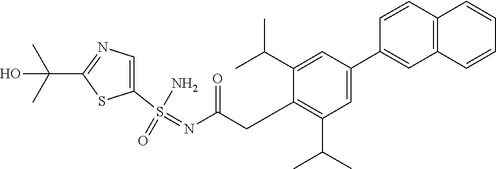 [SMILES: CC(C)c1cc(-c2ccc3ccccc3c2)cc(C(C)C)c1CC(=O)N=S(N)(=O)c1cnc(C(C)(C)O)s1] | N-(amino(2-(2-hydroxypropan- 2-yl)thiazol-5-yl)(oxo)- λ⁶-sulfaneylidene)-2-(2,6- diisopropyl-4-(naphthalen-2- yl)phenyl)acetamide | 550 |
| 114 | 248 | 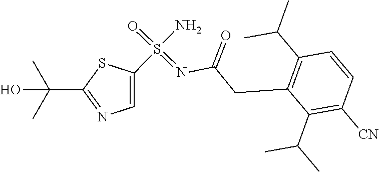 [SMILES: CC(C)c1ccc(C#N)c(C(C)C)c1CC(=O)N=S(N)(=O)c1cnc(C(C)(C)O)s1] | N-(amino(2-(2-hydroxypropan- 2-yl)thiazol-5-yl)(oxo)- λ⁶-sulfaneylidene)-2-(3-cyano-2,6- diisopropylphenyl)acetamide | 449 |
| 115 | 247 | 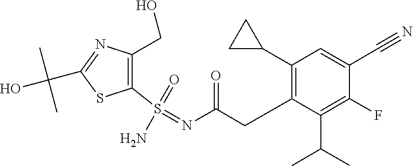 [SMILES: CC(C)c1c(F)c(C#N)cc(C2CC2)c1CC(=O)N=S(N)(=O)c1sc(C(C)(C)O)nc1CO] | N-(amino(4-(hydroxymethyl)- 2-(2-hydroxypropan-2- yl)thiazol-5-yl)(oxo)-λ⁶- sulfaneylidene)-2-(4-cyano-6- cyclopropyl-3-fluoro-2- isopropylphenyl)acetamide | 495 |
| 116 | 246 | 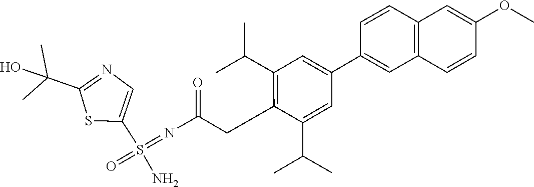 [SMILES: COc1ccc2cc(-c3cc(C(C)C)c(CC(=O)N=S(N)(=O)c4cnc(C(C)(C)O)s4)c(C(C)C)c3)ccc2c1] | N-(amino(2-(2-hydroxypropan- 2-yl)thiazol-5-yl)(oxo)- λ⁶-sulfaneylidene)-2- (2,6-diisopropyl-4-(6- methoxynaphthalen-2-yl) phenyl)acetamide | 580 |
| 117 | 309 | 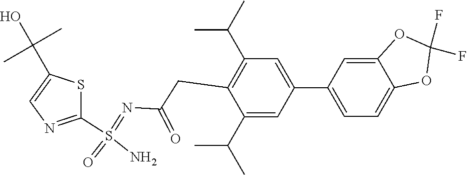 [SMILES: CC(C)c1cc(-c2ccc3c(c2)OC(F)(F)O3)cc(C(C)C)c1CC(=O)N=S(N)(=O)c1ncc(C(C)(C)O)s1] | N-(amino(5-(2-hydroxypropan- 2-yl)thiazol-2-yl)(oxo)- λ⁶-sulfaneylidene)-2-(4-(2,2- difluorobenzo[d][1,3]dioxol- 5-yl)-2,6-diisopropylphenyl) acetamide | 580 |
| 118 | 310 | 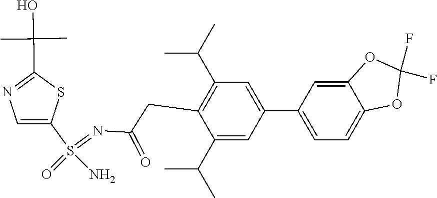 [SMILES: CC(C)c1cc(-c2ccc3c(c2)OC(F)(F)O3)cc(C(C)C)c1CC(=O)N=S(N)(=O)c1cnc(C(C)(C)O)s1] | N-(amino(2-(2-hydroxypropan- 2-yl)thiazol-5-yl)(oxo)- λ⁶-sulfaneylidene)-2-(4-(2,2- difluorobenzo[d][1,3]dioxol- 5-yl)-2,6-diisopropylphenyl) acetamide | 580 |
| 119 | 243 | 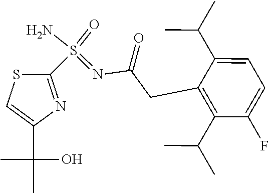 [SMILES: CC(C)c1ccc(F)c(C(C)C)c1CC(=O)N=S(N)(=O)c1nc(C(C)(C)O)cs1] | N-(amino(4-(2-hydroxypropan- 2-yl)thiazol-2-yl)(oxo)- λ⁶-sulfaneylidene)-2-(3-fluoro-2,6- diisopropylphenyl)acetamide | 442 |
| 120 | 242 | 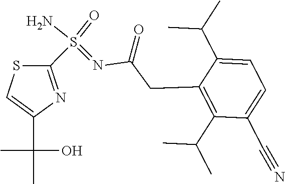 [SMILES: CC(C)c1ccc(C#N)c(C(C)C)c1CC(=O)N=S(N)(=O)c1nc(C(C)(C)O)cs1] | N-(amino(4-(2-hydroxypropan- 2-yl)thiazol-2-yl)(oxo)- λ⁶-sulfaneylidene)-2-(3-cyano-2,6- diisopropylphenyl)acetamide | 449 |
| 121 | 239 | 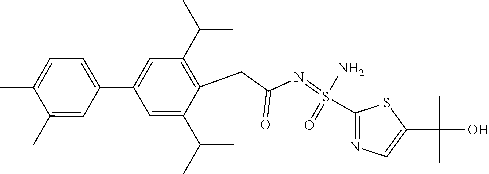 [SMILES: Cc1ccc(-c2cc(C(C)C)c(CC(=O)N=S(N)(=O)c3ncc(C(C)(C)O)s3)c(C(C)C)c2)cc1C] | N-(amino(5-(2-hydroxypropan- 2-yl)thiazol-2-yl)(oxo)- λ⁶-sulfaneylidene)-2-(3,5- diisopropyl-3′,4′-dimethyl- [1,1′-biphenyl]-4-yl)acetamide | 528 |
| 122 | 238 | 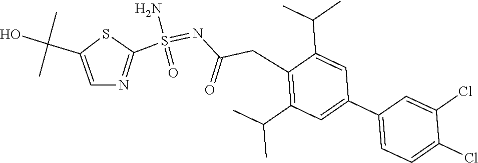 [SMILES: CC(C)c1cc(-c2ccc(Cl)c(Cl)c2)cc(C(C)C)c1CC(=O)N=S(N)(=O)c1ncc(C(C)(C)O)s1] | N-(amino(5-(2-hydroxypropan- 2-yl)thiazol-2-yl)(oxo)- λ⁶-sulfaneylidene)-2-(3′,4′- dichloro-3,5-diisopropyl- [1,1′-biphenyl]-4-yl)acetamide | 568 |
| 123 | 237 | 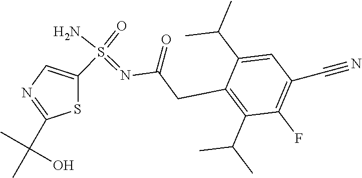 [SMILES: CC(C)c1cc(C#N)c(F)c(C(C)C)c1CC(=O)N=S(N)(=O)c1cnc(C(C)(C)O)s1] | N-(amino(2-(2-hydroxypropan- 2-yl)thiazol-5-yl)(oxo)- λ⁶-sulfaneylidene)-2- (4-cyano-3-fluoro-2,6- diisopropylphenyl)acetamide | 467 |
| 124 | 236 | 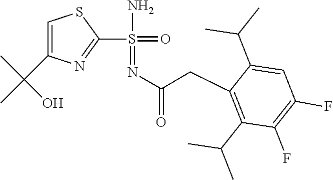 [SMILES: CC(C)c1cc(F)c(F)c(C(C)C)c1CC(=O)N=S(N)(=O)c1nc(C(C)(C)O)cs1] | N-(amino(4-(2-hydroxypropan- 2-yl)thiazol-2-yl)(oxo)- λ⁶-sulfaneylidene)-2- (3,4-difluoro-2,6- diisopropylphenyl)acetamide | 460 |
| 125 | 234 | 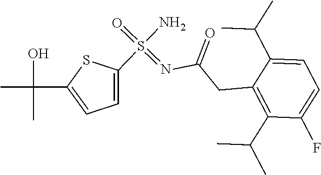 [SMILES: CC(C)c1ccc(F)c(C(C)C)c1CC(=O)N=S(N)(=O)c1ccc(C(C)(C)O)s1] | N-(amino(5-(2-hydroxypropan- 2-yl)thiophen-2-yl)(oxo)- λ⁶-sulfaneylidene)-2-(3-fluoro-2,6- diisopropylphenyl)acetamide | 441 |
| 126 | 233 | 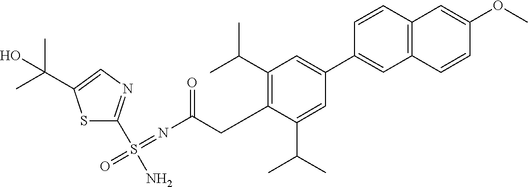 [SMILES: COc1ccc2cc(-c3cc(C(C)C)c(CC(=O)N=S(N)(=O)c4ncc(C(C)(C)O)s4)c(C(C)C)c3)ccc2c1] | N-(amino(5-(2-hydroxypropan- 2-yl)thiazol-2-yl)(oxo)- λ⁶-sulfaneylidene)-2- (2,6-diisopropyl-4-(6- methoxynaphthalen-2-yl) phenyl)acetamide | 580 |
| 127 | 232 | 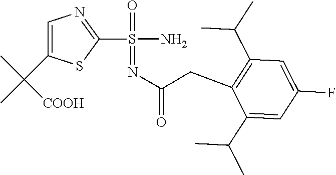 [SMILES: CC(C)c1cc(F)cc(C(C)C)c1CC(=O)N=S(N)(=O)c1ncc(C(C)(C)C(=O)O)s1] | 2-(2-(N′-(2-(4-fluoro-2,6- diisopropylphenyl)acetyl) sulfamidimidoyl)thiazol-5-yl)- 2-methylpropanoic acid | 470 |
| 128 | 120 | 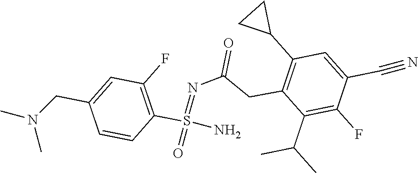 [SMILES: CC(C)c1c(F)c(C#N)cc(C2CC2)c1CC(=O)N=S(N)(=O)c1ccc(CN(C)C)cc1F] | N-(amino(4- ((dimethylamino)methyl)-2- fluorophenyl)(oxo)-λ⁶- sulfaneylidene)-2-(4-cyano-6- cyclopropyl-3-fluoro-2- isopropylphenyl)acetamide | 475 |
| 129 | 229 | 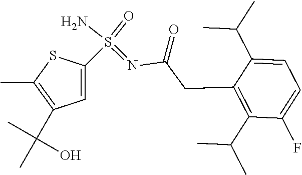 [SMILES: Cc1sc(S(N)(=O)=NC(=O)Cc2c(C(C)C)ccc(F)c2C(C)C)cc1C(C)(C)O] | N-(amino(4-(2-hydroxypropan- 2-yl)-5-methylthiophen-2- yl)(oxo)-λ⁶-sulfaneylidene)- 2-(3-fluoro-2,6- diisopropylphenyl)acetamide | 455 |
| 130 | 228 | 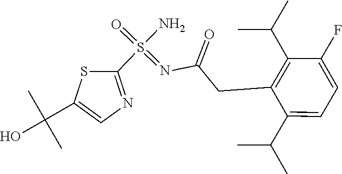 [SMILES: CC(C)c1ccc(F)c(C(C)C)c1CC(=O)N=S(N)(=O)c1ncc(C(C)(C)O)s1] | N-(amino(5-(2-hydroxypropan- 2-yl)thiazol-2-yl)(oxo)- λ⁶-sulfaneylidene)- 2-(3-fluoro-2,6- diisopropylphenyl)acetamide | 442 |
| 132 | 227 | 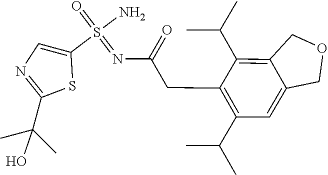 [SMILES: CC(C)c1cc2c(c(C(C)C)c1CC(=O)N=S(N)(=O)c1cnc(C(C)(C)O)s1)COC2] | N-(amino(2-(2-hydroxypropan- 2-yl)thiazol-5-yl)(oxo)- λ⁶-sulfaneylidene)-2- (4,6-diisopropyl-1,3- dihydroisobenzofuran-5- yl)acetamide | 466 |
| 133 | 226 | 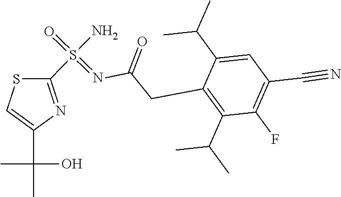 [SMILES: CC(C)c1cc(C#N)c(F)c(C(C)C)c1CC(=O)N=S(N)(=O)c1nc(C(C)(C)O)cs1] | N-(amino(4-(2-hydroxypropan- 2-yl)thiazol-2-yl)(oxo)- λ⁶-sulfaneylidene)-2-(4- cyano-3-fluoro-2,6- diisopropylphenyl)acetamide | 467 |
| 134 | 225 | 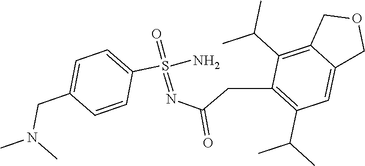 [SMILES: CC(C)c1cc2c(c(C(C)C)c1CC(=O)N=S(N)(=O)c1ccc(CN(C)C)cc1)COC2] | N-(amino(4-((dimethylamino) methyl)phenyl)(oxo)-λ⁶- sulfaneylidene)-2- (4,6-diisopropyl-1,3- dihydroisobenzofuran-5-yl) acetamide | 458 |
| 135 | 224 | 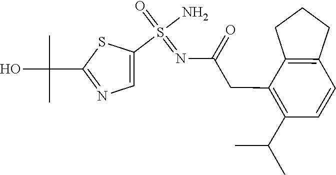 [SMILES: CC(C)c1ccc2c(c1CC(=O)N=S(N)(=O)c1cnc(C(C)(C)O)s1)CCC2] | N-(amino(2-(2-hydroxypropan- 2-yl)thiazol-5-yl)(oxo)- λ⁶-sulfaneylidene)-2-(5-isopropyl- 2,3-dihydro-1H-inden- 4-yl)acetamide | 422 |
| 136 | 191 | 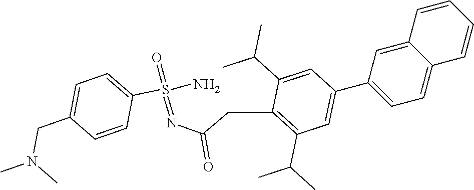 [SMILES: CC(C)c1cc(-c2ccc3ccccc3c2)cc(C(C)C)c1CC(=O)N=S(N)(=O)c1ccc(CN(C)C)cc1] | N-(amino(4-((dimethylamino) methyl)phenyl)(oxo)-λ⁶- sulfaneylidene)-2-(2,6- diisopropyl-4-(naphthalen-2- yl)phenyl)acetamide | 542 |
| 137 | 222 | 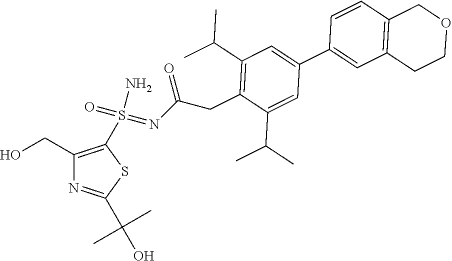 [SMILES: CC(C)c1cc(-c2ccc3c(c2)CCOC3)cc(C(C)C)c1CC(=O)N=S(N)(=O)c1sc(C(C)(C)O)nc1CO] | N-(amino(4-(hydroxymethyl)- 2-(2-hydroxypropan-2- yl)thiazol-5-yl)(oxo)-λ⁶- sulfaneylidene)-2-(4- (isochroman-6-yl)-2,6- diisopropylphenyl)acetamide | 586 |
| 138 | 307 | 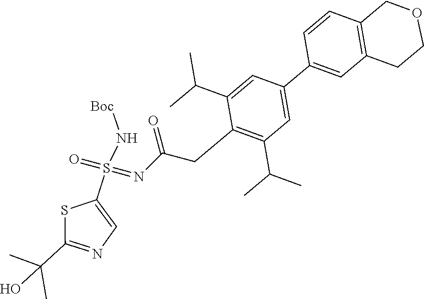 [SMILES: CC(C)c1cc(-c2ccc3c(c2)CCOC3)cc(C(C)C)c1CC(=O)N=S(=O)(NC(=O)OC(C)(C)C)c1cnc(C(C)(C)O)s1] | tert-butyl (2-(2- hydroxypropan-2-yl)-N-(2-(4- (isochroman-6-yl)-2,6- diisopropylphenyl)acetyl)thiazole- 5-sulfonimidoyl)carbamate | 656 |
| 139 | 231 | 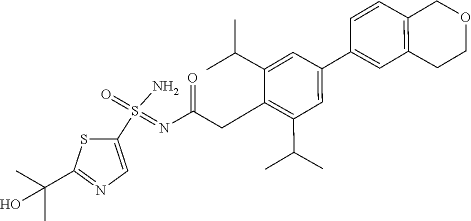 [SMILES: CC(C)c1cc(-c2ccc3c(c2)CCOC3)cc(C(C)C)c1CC(=O)N=S(N)(=O)c1cnc(C(C)(C)O)s1] | N-(amino(2-(2-hydroxypropan- 2-yl)thiazol-5-yl)(oxo)- λ⁶-sulfaneylidene)-2-(4- (isochroman-6-yl)-2,6- diisopropylphenyl)acetamide | 556 |
| 140 | 306 | 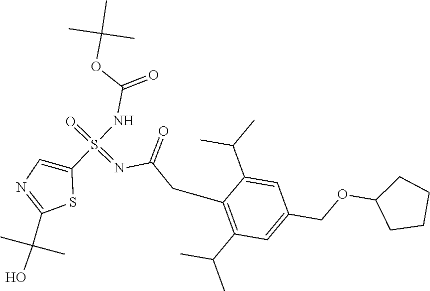 [SMILES: CC(C)c1cc(COC2CCCC2)cc(C(C)C)c1CC(=O)N=S(=O)(NC(=O)OC(C)(C)C)c1cnc(C(C)(C)O)s1] | tert-butyl (N-(2-(4- ((cyclopentyloxy)methyl)-2,6 diisopropylphenyl)acetyl)- 2-(2-hydroxypropan-2 yl)thiazole-5-sulfonimidoyl) carbamate | 622 |
| 141 | 305 | 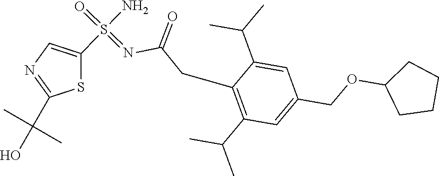 [SMILES: CC(C)c1cc(COC2CCCC2)cc(C(C)C)c1CC(=O)N=S(N)(=O)c1cnc(C(C)(C)O)s1] | N-(amino(2-(2-hydroxypropan- 2-yl)thiazol-5-yl)(oxo)- λ⁶-sulfaneylidene)-2-(4- ((cyclopentyloxy)methyl)-2,6- diisopropylphenyl)acetamide | 522 |
| 142 | 216 | 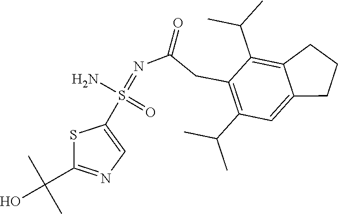 | N-(amino(2-(2-hydroxypropan- 2-yl)thiazol-5-yl)(oxo)- λ⁶-sulfaneylidene)-2-(4,6- diisopropyl-2,3-dihydro-1H- inden-5-yl)acetamide | 464 |
| 143 | 215 | 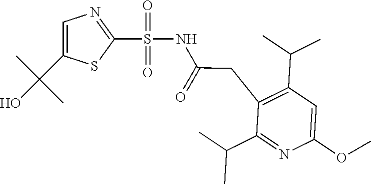 [SMILES: COc1cc(C(C)C)c(CC(=O)NS(=O)(=O)c2ncc(C(C)(C)O)s2)c(C(C)C)n1] | 2-(2,4-diisopropyl-6- methoxypyridin-3-yl)-N-((5-(2- hydroxypropan-2-yl)thiazol- 2-yl)sulfonyl)acetamide | 456 |
| 144 | 203 | 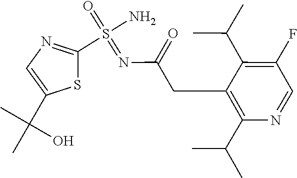 [SMILES: CC(C)c1ncc(F)c(C(C)C)c1CC(=O)N=S(N)(=O)c1ncc(C(C)(C)O)s1] | N-(amino(5-(2-hydroxypropan- 2-yl)thiazol-2-yl)(oxo)- λ⁶-sulfaneylidene)-2-(5-fluoro- 2,4-diisopropylpyridin-3- yl)acetamide | 443 |
| 145 | 213 | 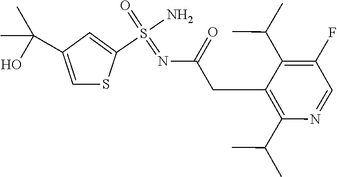 [SMILES: CC(C)c1ncc(F)c(C(C)C)c1CC(=O)N=S(N)(=O)c1cc(C(C)(C)O)cs1] | N-(amino(4-(2-hydroxypropan- 2-yl)thiophen-2-yl)(oxo)- λ⁶-sulfaneylidene)-2-(5-fluoro- 2,4-diisopropylpyridin-3- yl)acetamide | 442 |
| 146 | 204 | 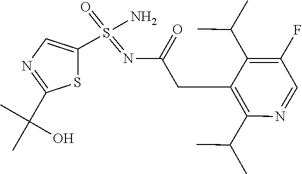 [SMILES: CC(C)c1ncc(F)c(C(C)C)c1CC(=O)N=S(N)(=O)c1cnc(C(C)(C)O)s1] | N-(amino(2-(2-hydroxypropan- 2-yl)thiazol-5-yl)(oxo)- λ⁶-sulfaneylidene)-2-(5-fluoro- 2,4-diisopropylpyridin-3- yl)acetamide | 443 |
| 147 | 202 | 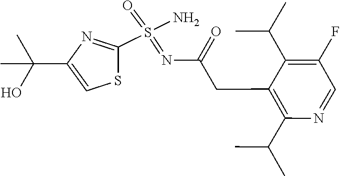 [SMILES: CC(C)c1ncc(F)c(C(C)C)c1CC(=O)N=S(N)(=O)c1nc(C(C)(C)O)cs1] | N-(amino(4-(2-hydroxypropan- 2-yl)thiazol-2-yl)(oxo)- λ⁶-sulfaneylidene)-2-(5-fluoro- 2,4-diisopropylpyridin-3- yl)acetamide | 443 |
| 148 | 211 | 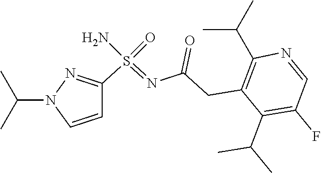 [SMILES: CC(C)c1ncc(F)c(C(C)C)c1CC(=O)N=S(N)(=O)c1ccn(C(C)C)n1] | N-(amino(1-isopropyl- 1H-pyrazol-3-yl)(oxo)-λ⁶- sulfaneylidene)-2-(5-fluoro- 2,4-diisopropylpyridin-3- yl)acetamide | 410 |
| 149 | 210 | 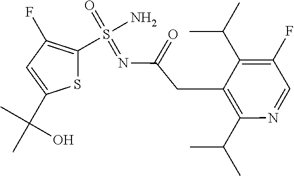 [SMILES: CC(C)c1ncc(F)c(C(C)C)c1CC(=O)N=S(N)(=O)c1sc(C(C)(C)O)cc1F] | N-(amino(3-fluoro-5-(2- hydroxypropan-2-yl)thiophen-2- yl)(oxo)-λ⁶-sulfaneylidene)- 2-(5-fluoro-2,4- diisopropylpyridin-3-yl) acetamide | 460 |
| 150 | 209 | 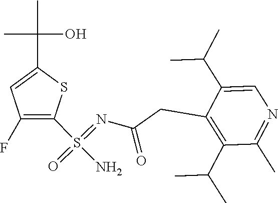 [SMILES: Cc1ncc(C(C)C)c(CC(=O)N=S(N)(=O)c2sc(C(C)(C)O)cc2F)c1C(C)C] | N-(amino(3-fluoro-5-(2- hydroxypropan-2-yl)thiophen-2- yl)(oxo)-λ⁶-sulfaneylidene)- 2-(3,5-diisopropyl-2- methylpyridin-4-yl)acetamide | 456 |
| 151 | 208 | 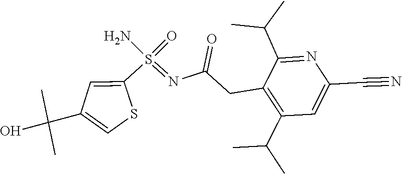 [SMILES: CC(C)c1cc(C#N)nc(C(C)C)c1CC(=O)N=S(N)(=O)c1cc(C(C)(C)O)cs1] | N-(amino(4-(2-hydroxypropan- 2-yl)thiophen-2-yl)(oxo)- λ⁶-sulfaneylidene)-2-(6-cyano- 2,4-diisopropylpyridin-3- yl)acetamide | 449 |
| 152 | 207 | 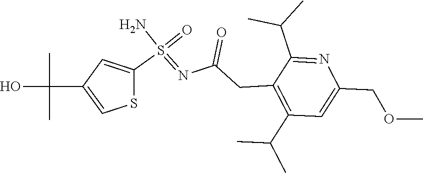 [SMILES: COCc1cc(C(C)C)c(CC(=O)N=S(N)(=O)c2cc(C(C)(C)O)cs2)c(C(C)C)n1] | N-(amino(4-(2-hydroxypropan- 2-yl)thiophen-2-yl)(oxo)- λ⁶-sulfaneylidene)-2- (2,4-diisopropyl-6- (methoxymethyl)pyridin-3- yl)acetamide | 468 |
| 153 | 206 | 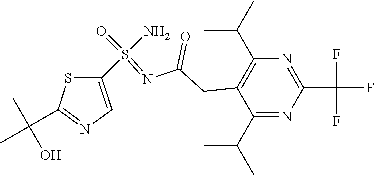 [SMILES: CC(C)c1nc(C(F)(F)F)nc(C(C)C)c1CC(=O)N=S(N)(=O)c1cnc(C(C)(C)O)s1] | N-(amino(2-(2-hydroxypropan- 2-yl)thiazol-5-yl)(oxo)- λ⁶-sulfaneylidene)-2- (4,6-diisopropyl-2- (trifluoromethyl)pyrimidin-5- yl)acetamide | 494 |
| 154 | 205 | 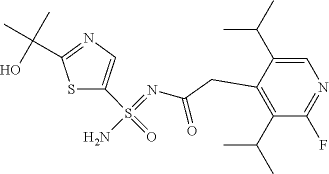 [SMILES: CC(C)c1cnc(F)c(C(C)C)c1CC(=O)N=S(N)(=O)c1cnc(C(C)(C)O)s1] | N-(amino(2-(2-hydroxypropan- 2-yl)thiazol-5-yl)(oxo)- λ⁶-sulfaneylidene)-2-(2-fluoro- 3,5-diisopropylpyridin-4- yl)acetamide | 443 |
| 220 | 263 | 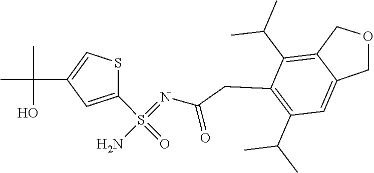 [SMILES: CC(C)c1cc2c(c(C(C)C)c1CC(=O)N=S(N)(=O)c1cc(C(C)(C)O)cs1)COC2] | N-(amino(4-(2-hydroxypropan- 2-yl)thiophen-2-yl)(oxo)- λ⁶-sulfaneylidene)-2- (4,6-diisopropyl-1,3- dihydroisobenzofuran-5- yl)acetamide | 465 |
| 221 | 264 | 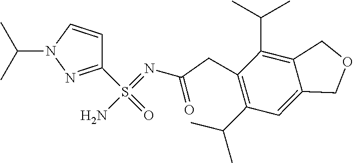 [SMILES: CC(C)c1cc2c(c(C(C)C)c1CC(=O)N=S(N)(=O)c1ccn(C(C)C)n1)COC2] | N-(amino(1-isopropyl-1H- pyrazol-3-yl)(oxo)-λ⁶- sulfaneylidene)-2- (4,6-diisopropyl-1,3- dihydroisobenzofuran-5- yl)acetamide | 433 |

Examples in the following table were prepared using similar conditions as described in Example 4 and Scheme 2 from appropriate starting materials.

TABLE 22

| | | | | | | LC- |
|---|---|---|---|---|---|---|
| | | | | | | MS |
| Ex. | Cmpd | | | | Elu- | [M + |
| # | # | Structure | IUPAC Name | Column | ents | H]⁺ |
| 155 | 261b | 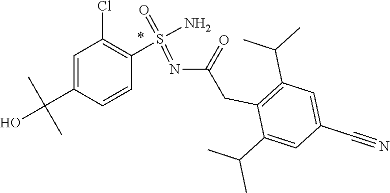 [SMILES: CC(C)c1cc(C#N)cc(C(C)C)c1CC(=O)N=S(N)(=O)c1ccc(C(C)(C)O)cc1Cl] | (R) or (S)-N-(amino (2-chloro-4-(2- hydroxypropan-2-yl) phenyl)(oxo)-λ⁶- sulfaneylidene)-2- (4-cyano-2,6- diisopropylphenyl) acetamide | CHIRAL- PAK ID, 2*25 cm (5 um) | IPA in Hex (0.1% FA) | 476 |
| 156 | 261a | 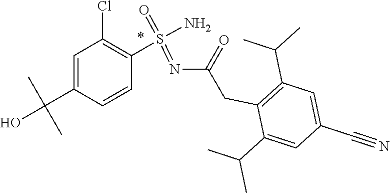 [SMILES: CC(C)c1cc(C#N)cc(C(C)C)c1CC(=O)N=S(N)(=O)c1ccc(C(C)(C)O)cc1Cl] | (S) or (R)-N-(amino (2-chloro-4-(2- hydroxypropan-2-yl) phenyl)(oxo)-λ⁶- sulfaneylidene)-2- (4-cyano-2,6- diisopropylphenyl) acetamide | CHIRAL- PAK ID, 2*25 cm (5 um) | IPA in Hex (0.1% FA) | 476 |
| 157 | 256a | 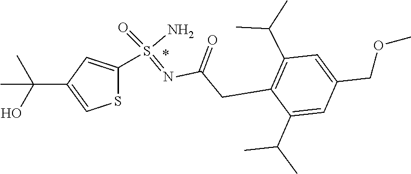 [SMILES: COCc1cc(C(C)C)c(CC(=O)N=S(N)(=O)c2cc(C(C)(C)O)cs2)c(C(C)C)c1] | (R) or (S)- N-(amino(4- (2-hydroxypropan-2- yl)thiophen-2- yl)(oxo)-λ⁶- sulfaneylidene)- 2-(2,6- diisopropyl-4- (methoxymethyl) phenyl)acetamide | CHIRAL- PAK ID, 2*25 cm (5 um) | IPA in Hex: DC M = 5:1 | 465 (M − 1) |
| 158 | 256b | 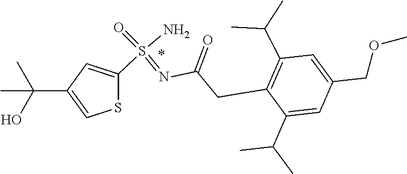 [SMILES: COCc1cc(C(C)C)c(CC(=O)N=S(N)(=O)c2cc(C(C)(C)O)cs2)c(C(C)C)c1] | (S) or (R)- N-(amino(4- (2-hydroxypropan-2- yl)thiophen-2- yl)(oxo)-λ⁶- sulfaneylidene)- 2-(2,6- diisopropyl-4- (methoxymethyl) phenyl)acetamide | CHIRAL- PAK ID, 2*25 cm (5 um) | IPA in Hex: DC M = 5:1 | 465 (M − 1) |
| 159 | 201b | 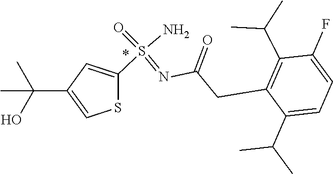 [SMILES: CC(C)c1ccc(F)c(C(C)C)c1CC(=O)N=S(N)(=O)c1cc(C(C)(C)O)cs1] | (R) or (S)- N-(amino(4- (2-hydroxypropan-2- yl)thiophen-2- yl)(oxo)-λ⁶- sulfaneylidene)-2-(3- fluoro-2,6- diisopropylphenyl) acetamide | CHIRAL- PAK ID, 2*25 cm (5 um) | IPA in Hex: DC M = 5:1 | 441 |
| 160 | 201a | 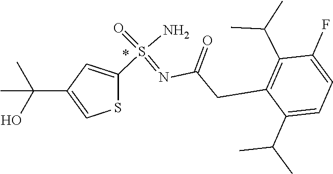 [SMILES: CC(C)c1ccc(F)c(C(C)C)c1CC(=O)N=S(N)(=O)c1cc(C(C)(C)O)cs1] | (S) or (R)- N-(amino(4- (2-hydroxypropan-2- yl)thiophen-2- yl)(oxo)-λ⁶- sulfaneylidene)-2-(3- fluoro-2,6- diisopropylphenyl) acetamide | CHIRAL- PAK ID, 2*25 cm (5 um) | IPA in Hex: DC M = 5:1 | 441 |
| 161 | 258a | 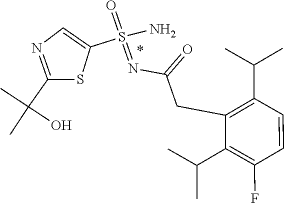 [SMILES: CC(C)c1ccc(F)c(C(C)C)c1CC(=O)N=S(N)(=O)c1cnc(C(C)(C)O)s1] | (R) or (S)-N-(amino(2- (2-hydroxypropan-2- yl)thiazol-5- yl)(oxo)-λ⁶- sulfaneylidene)-2-(3- fluoro-2,6- diisopropylphenyl) acetamide | Chiralpak ID, 2*25 cm, 5 um | IPA in Hex (0.1% FA) | 440 (M − 1) |
| 162 | 258b | 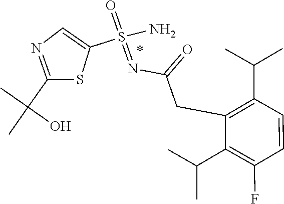 [SMILES: CC(C)c1ccc(F)c(C(C)C)c1CC(=O)N=S(N)(=O)c1cnc(C(C)(C)O)s1] | (S) or (R)-N-(amino(2- (2-hydroxypropan-2- yl)thiazol-5-yl)(oxo)-λ⁶- sulfaneylidene)-2-(3- fluoro-2,6- diisopropylphenyl) acetamide | Chiralpak ID, 2*25 cm, 5 um | IPA in Hex (0.1% FA) | 440 (M − 1) |
| 163 | 249a | 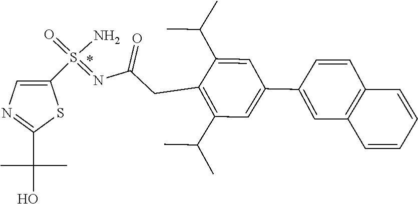 [SMILES: CC(C)c1cc(-c2ccc3ccccc3c2)cc(C(C)C)c1CC(=O)N=S(N)(=O)c1cnc(C(C)(C)O)s1] | (R) or (S)- N-(amino(2- (2-hydroxypropan-2- yl)thiazol-5- yl)(oxo)-λ⁶- sulfaneylidene)- 2-(2,6- diisopropyl-4- (naphthalen- 2-yl)phenyl) acetamide | Chiralpak ID, 2*25 cm, 5 um | IPA in Hex (0.1% FA) | 550 |
| 164 | 249b | 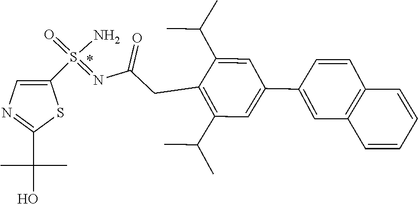 [SMILES: CC(C)c1cc(-c2ccc3ccccc3c2)cc(C(C)C)c1CC(=O)N=S(N)(=O)c1cnc(C(C)(C)O)s1] | (S) or (R)- N-(amino(2- (2-hydroxypropan-2- yl)thiazol-5- yl)(oxo)-λ⁶- sulfaneylidene)- 2-(2,6- diisopropyl-4- (naphthalen- 2-yl)phenyl) acetamide | Chiralpak ID, 2*25 cm, 5 um | IPA in Hex (0.1% FA) | 550 |
| 165 | 309b | 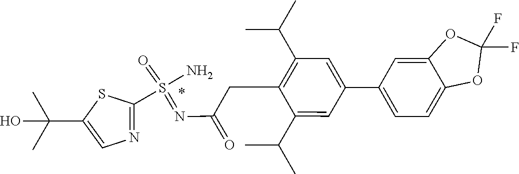 [SMILES: CC(C)c1cc(-c2ccc3c(c2)OC(F)(F)O3)cc(C(C)C)c1CC(=O)N=S(N)(=O)c1ncc(C(C)(C)O)s1] | (S) or (R)- N-(amino(5- (2-hydroxypropan-2- yl)thiazol-2- yl)(oxo)-λ⁶- sulfaneylidene)-2-(4- (2,2-difluorobenzo[d] [1,3]dioxol-5-yl)-2,6- diisopropylphenyl) acetamide | CHIRAL- PAK IF, 2*25 cm, 5 um | EtOH in Hex (0.1% FA) | 580 |
| 166 | 309a | 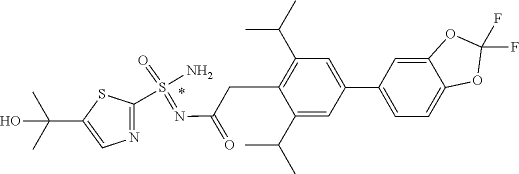 [SMILES: CC(C)c1cc(-c2ccc3c(c2)OC(F)(F)O3)cc(C(C)C)c1CC(=O)N=S(N)(=O)c1ncc(C(C)(C)O)s1] | (R) or (S)- N-(amino(5- (2-hydroxypropan-2- yl)thiazol-2- yl)(oxo)-λ⁶- sulfaneylidene)-2-(4- (2,2-difluorobenzo[d] [1,3]dioxol-5-yl)-2,6- diisopropylphenyl) acetamide | CHIRAL- PAK IF, 2*25 cm, 5 um | EtOH in Hex (0.1% FA) | 580 |
| 167 | 239a | 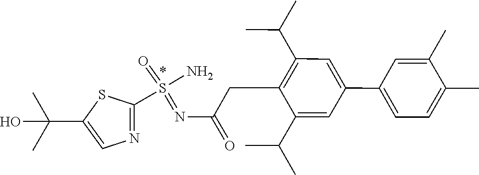 [SMILES: Cc1ccc(-c2cc(C(C)C)c(CC(=O)N=S(N)(=O)c3ncc(C(C)(C)O)s3)c(C(C)C)c2)cc1C] | (S) or (R)-N-(amino(5- (2-hydroxypropan-2- yl)thiazol-2- yl)(oxo)-λ⁶- sulfaneylidene)-2-(3,5- diisopropyl-3′,4′- dimethyl- [1,1′-biphenyl]-4- yl)acetamide | CHIRAL- PAK IF, 2*25 cm, 5 um | EtOH in Hex (0.1% FA) | 528 |
| 168 | 239b | 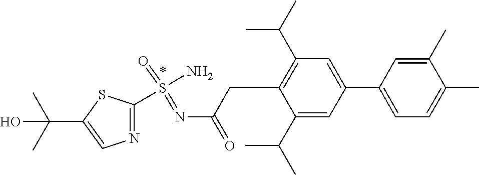 [SMILES: Cc1ccc(-c2cc(C(C)C)c(CC(=O)N=S(N)(=O)c3ncc(C(C)(C)O)s3)c(C(C)C)c2)cc1C] | (R) or (S)- N-(amino(5- (2-hydroxypropan-2- yl)thiazol-2- yl)(oxo)-λ⁶- sulfaneylidene)-2-(3,5- diisopropyl-3′,4'- dimethyl- [1,1′-biphenyl]-4- yl)acetamide | CHIRAL- PAK IF, 2*25 cm, 5 um | EtOH in Hex (0.1% FA) | 528 |
| 169 | 238a | 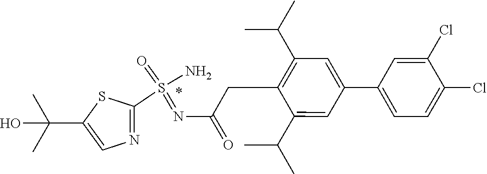 [SMILES: CC(C)c1cc(-c2ccc(Cl)c(Cl)c2)cc(C(C)C)c1CC(=O)N=S(N)(=O)c1ncc(C(C)(C)O)s1] | (S) or (R)-N-(amino(5- (2-hydroxypropan-2- yl)thiazol-2- yl)(oxo)-λ⁶- sulfaneylidene)- 2-(3′,4′- dichloro-3,5- diisopropyl- [1,1′-biphenyl]-4- yl)acetamide | CHIRAL- PAK IF, 2*25 cm, 5 um | IPA in Hex (0.1% FA) | 568 |
| 170 | 238b | 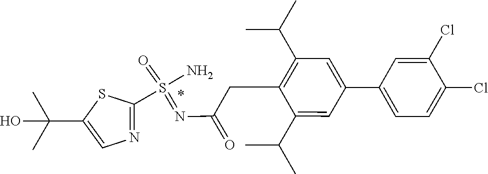 [SMILES: CC(C)c1cc(-c2ccc(Cl)c(Cl)c2)cc(C(C)C)c1CC(=O)N=S(N)(=O)c1ncc(C(C)(C)O)s1] | (R) or (S)- N-(amino(5- (2-hydroxypropan-2- yl)thiazol-2- yl)(oxo)-λ⁶- sulfaneylidene)- 2-(3′,4′- dichloro-3,5- diisopropyl- [1,1′-biphenyl]-4- yl)acetamide | CHIRAL- PAK IF, 2*25 cm, 5 um | IPA in Hex (0.1% FA) | 568 |
| 171 | 221a | 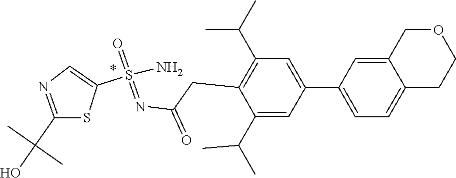 [SMILES: CC(C)c1cc(-c2ccc3c(c2)COCC3)cc(C(C)C)c1CC(=O)N=S(N)(=O)c1cnc(C(C)(C)O)s1] | (R) or (S)- N-(amino(2- (2-hydroxypropan-2- yl)thiazol-5- yl)(oxo)-λ⁶- sulfaneylidene)-2-(4- (isochroman- 7-yl)-2,6- diisopropylphenyl) acetamide | CHIRAL- PAK IF, 2*25 cm, 5 um | EtOH in Hex (0.1% FA) | 556 |
| 172 | 221b | 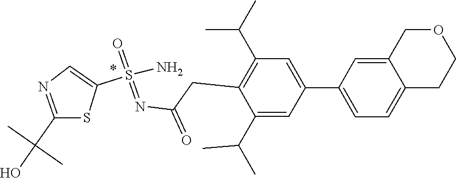 [SMILES: CC(C)c1cc(-c2ccc3c(c2)COCC3)cc(C(C)C)c1CC(=O)N=S(N)(=O)c1cnc(C(C)(C)O)s1] | (S) or (R)- N-(amino(2-(2- hydroxypropan-2- yl)thiazol-5- yl)(oxo)-λ⁶- sulfaneylidene)-2-(4- (isochroman- 7-yl)-2,6- diisopropylphenyl) acetamide | CHIRAL- PAK IF, 2*25 cm, 5 um | EtOH in Hex (0.1% FA) | 556 |
| 173 | 229a | 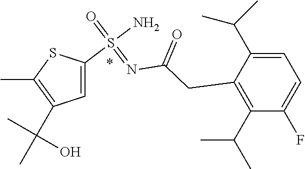 [SMILES: Cc1sc(S(N)(=O)=NC(=O)Cc2c(C(C)C)ccc(F)c2C(C)C)cc1C(C)(C)O] | (R) or (S)- N-(amino(4- (2-hydroxypropan- 2-yl)- 5-methylthiophen- 2-yl)(oxo)- λ⁶-sulfaneylidene)- 2-(3-fluoro-2,6- diisopropylphenyl) acetamide | CHIRAL- PAK IC, 2*25 cm, 5 um | IPA in Hex (0.1% FA) | 455 |
| 174 | 229b | 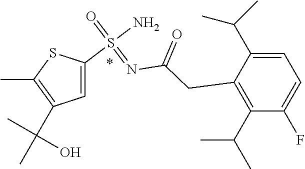 [SMILES: Cc1sc(S(N)(=O)=NC(=O)Cc2c(C(C)C)ccc(F)c2C(C)C)cc1C(C)(C)O] | (S) or (R)- N-(amino(4- (2-hydroxypropan- 2-yl)- 5-methylthiophen- 2-yl)(oxo)- λ⁶-sulfaneylidene)- 2-(3-fluoro-2,6- diisopropylphenyl) acetamide | CHIRAL- PAK IC, 2*25 cm, 5 um | IPA in Hex (0.1% FA) | 455 |
| 175 | 234b | 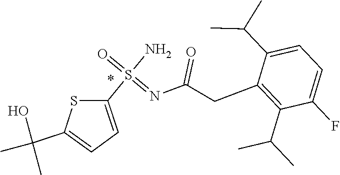 [SMILES: CC(C)c1ccc(F)c(C(C)C)c1CC(=O)N=S(N)(=O)c1ccc(C(C)(C)O)s1] | (S) or (R)- N-(amino(5- (2-hydroxypropan-2- yl)thiophen-2- yl)(oxo)-λ⁶- sulfaneylidene)-2-(3- fluoro-2,6- diisopropylphenyl) acetamide | Chiralpak ID, 2*25 cm, 5 um | IPA in Hex (0.1% FA) | 441 |
| 176 | 234a | 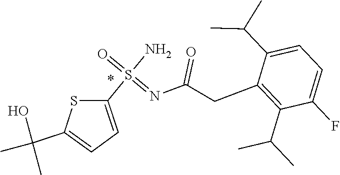 [SMILES: CC(C)c1ccc(F)c(C(C)C)c1CC(=O)N=S(N)(=O)c1ccc(C(C)(C)O)s1] | (R) or (S)- N-(amino(5- (2-hydroxypropan-2- yl)thiophen-2- yl)(oxo)-λ⁶- sulfaneylidene)-2-(3- fluoro-2,6- diisopropylphenyl) acetamide | Chiralpak ID, 2*25 cm, 5 um | IPA in Hex (0.1% FA) | 441 |
| 177 | 236a | 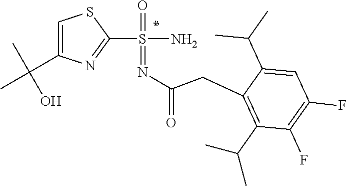 [SMILES: CC(C)c1cc(F)c(F)c(C(C)C)c1CC(=O)N=S(N)(=O)c1nc(C(C)(C)O)cs1] | (R) or (S)- N-(amino(4- (2-hydroxypropan-2- yl)thiazol-2- yl)(oxo)-λ⁶- sulfaneylidene)- 2-(3,4- difluoro-2,6- diisopropylphenyl) acetamide | Chiralpak ID, 2*25 cm, 5 um | IPA in Hex (0.1% FA) | 460 |
| 178 | 236b | 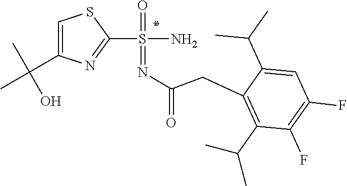 [SMILES: CC(C)c1cc(F)c(F)c(C(C)C)c1CC(=O)N=S(N)(=O)c1nc(C(C)(C)O)cs1] | (S) or (R)- N-(amino(4-(2- hydroxypropan-2- yl)thiazol-2- yl)(oxo)-λ⁶- sulfaneylidene)- 2-(3,4- difluoro-2,6- diisopropylphenyl) acetamide | Chiralpak ID, 2*25 cm, 5 um | IPA in Hex (0.1% FA) | 460 |
| 179 | 242a | 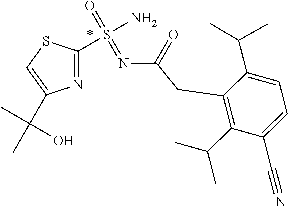 [SMILES: CC(C)c1ccc(C#N)c(C(C)C)c1CC(=O)N=S(N)(=O)c1nc(C(C)(C)O)cs1] | (R) or (S)- N-(amino(4- (2-hydroxypropan-2- yl)thiazol-2- yl)(oxo)-λ⁶- sulfaneylidene)-2-(3- cyano-2,6- diisopropylphenyl) acetamide | Chiralpak ID, 2*25 cm, 5 um | IPA in Hex (0.1% FA) | 449 |
| 180 | 242b | 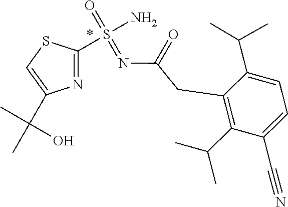 [SMILES: CC(C)c1ccc(C#N)c(C(C)C)c1CC(=O)N=S(N)(=O)c1nc(C(C)(C)O)cs1] | (S) or (R)-N-(amino(4- (2-hydroxypropan-2- yl)thiazol-2-yl)(oxo)-λ⁶- sulfaneylidene)-2-(3- cyano-2,6- diisopropylphenyl) acetamide | Chiralpak ID, 2*25 cm, 5 um | IPA in Hex (0.1% FA) | 449 |
| 181 | 243a | 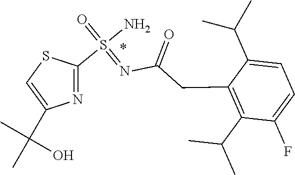 [SMILES: CC(C)c1ccc(F)c(C(C)C)c1CC(=O)N=S(N)(=O)c1nc(C(C)(C)O)cs1] | (R) or (S)- N-(amino(4- (2-hydroxypropan-2- yl)thiazol-2- yl)(oxo)-λ⁶- sulfaneylidene)-2-(3- fluoro-2,6- diisopropylphenyl) acetamide | Chiralpak ID, 2*25 cm, 5 um | IPA in Hex (0.1% FA) | 442 |
| 182 | 243b | 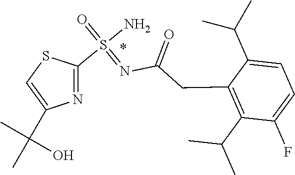 [SMILES: CC(C)c1ccc(F)c(C(C)C)c1CC(=O)N=S(N)(=O)c1nc(C(C)(C)O)cs1] | (S) or (R)-N-(amino(4- (2-hydroxypropan-2- yl)thiazol-2-yl)(oxo)-λ⁶- sulfaneylidene)-2- (3-fluoro-2,6- diisopropylphenyl) acetamide | Chiralpak ID, 2*25 cm, 5 um | IPA in Hex (0.1% FA) | 442 |
| 183 | 227a | 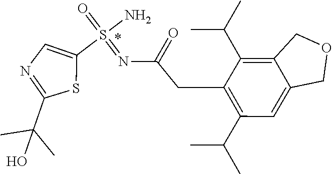 [SMILES: CC(C)c1cc2c(c(C(C)C)c1CC(=O)N=S(N)(=O)c1cnc(C(C)(C)O)s1)COC2] | (R) or (S)- N-(amino(2- (2-hydroxypropan-2- yl)thiazol-5- yl)(oxo)-λ⁶- sulfaneylidene)- 2-(4,6- diisopropyl-1,3- dihydro- isobenzofuran-5- yl)acetamide | Chiralpak IA, 2*25 cm, 5 um | EtOH in Hex (0.1% FA) | 466 |
| 184 | 227b | 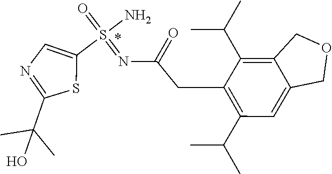 [SMILES: CC(C)c1cc2c(c(C(C)C)c1CC(=O)N=S(N)(=O)c1cnc(C(C)(C)O)s1)COC2] | (S) or (R)- N-(amino(2- (2-hydroxypropan-2- yl)thiazol-5- yl)(oxo)-λ⁶- sulfaneylidene)- 2-(4,6- diisopropyl-1,3- dihydro- isobenzofuran-5- yl)acetamide | Chiralpak IA, 2*25 cm, 5 um | EtOH in Hex (0.1% FA) | 466 |
| 185 | 251b | 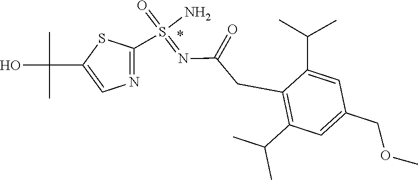 [SMILES: COCc1cc(C(C)C)c(CC(=O)N=S(N)(=O)c2ncc(C(C)(C)O)s2)c(C(C)C)c1] | (R) or (S)- N-(amino(5-(2- hydroxypropan-2- yl)thiazol-2- yl)(oxo)-λ⁶- sulfaneylidene)- 2-(2,6- diisopropyl-4- (methoxymethyl) phenyl)acetamide | Chiralpak ID, 2*25 cm, 5 um | IPA in Hex (0.1% FA) | 468 |
| 186 | 251a | 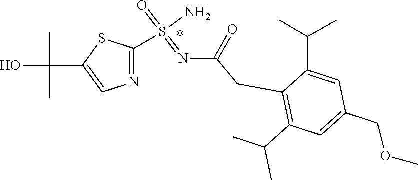 [SMILES: COCc1cc(C(C)C)c(CC(=O)N=S(N)(=O)c2ncc(C(C)(C)O)s2)c(C(C)C)c1] | (S) or (R)- N-(amino(5-(2- hydroxypropan-2- yl)thiazol-2- yl)(oxo)-λ⁶- sulfaneylidene)- 2-(2,6- diisopropyl-4- (methoxymethyl) phenyl)acetamide | Chiralpak ID, 2*25 cm, 5 um | IPA in Hex (0.1% FA) | 468 |
| 187 | 228a | 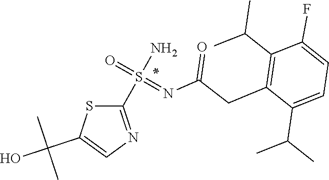 [SMILES: CC(C)c1ccc(F)c(C(C)C)c1CC(=O)N=S(N)(=O)c1ncc(C(C)(C)O)s1] | (S) or (R)- N-(amino(5-(2- hydroxypropan-2- yl)thiazol-2- yl)(oxo)-λ⁶- sulfaneylidene)-2-(3- fluoro-2,6- diisopropylphenyl) acetamide | Chiralpak ID, 2*25 cm, 5 um | IPA in Hex (0.1% FA) | 442 |
| 188 | 228b | 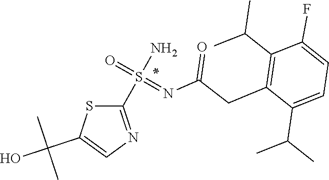 [SMILES: CC(C)c1ccc(F)c(C(C)C)c1CC(=O)N=S(N)(=O)c1ncc(C(C)(C)O)s1] | (R) or (S)- N-(amino(5-(2- hydroxypropan-2- yl)thiazol-2- yl)(oxo)-λ⁶- sulfaneylidene)-2-(3- fluoro-2,6- diisopropylphenyl) acetamide | Chiralpak ID, 2*25 cm, 5 um | IPA in Hex (0.1% FA) | 442 |
| 189 | 226a | 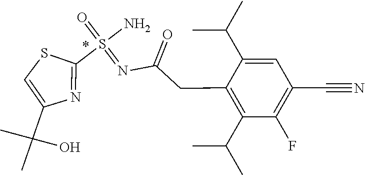 [SMILES: CC(C)c1cc(C#N)c(F)c(C(C)C)c1CC(=O)N=S(N)(=O)c1nc(C(C)(C)O)cs1] | (S) or (R)- N-(amino(4-(2- hydroxypropan-2- yl)thiazol-2- yl)(oxo)-λ⁶- sulfaneylidene)-2-(4- cyano-3-fluoro-2,6- diisopropylphenyl) acetamide | CHIRAL- PAK IG, 20*250 mm, 5 um | IPA in Hex (0.1% FA) | 467 |
| 190 | 226b | 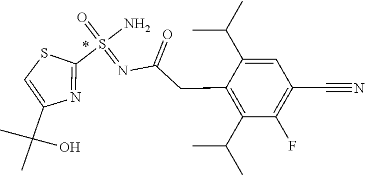 [SMILES: CC(C)c1cc(C#N)c(F)c(C(C)C)c1CC(=O)N=S(N)(=O)c1nc(C(C)(C)O)cs1] | (R) or (S)- N-(amino(4-(2- hydroxypropan-2- yl)thiazol-2- yl)(oxo)-λ⁶- sulfaneylidene)-2-(4- cyano-3-fluoro-2,6- diisopropylphenyl) acetamide | CHIRAL- PAK IG, 20*250 mm, 5 um | IPA in Hex (0.1% FA) | 467 |
| 191 | 225a | 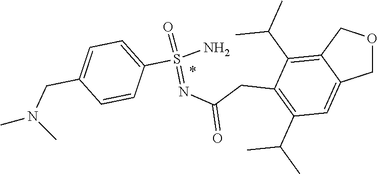 [SMILES: CC(C)c1cc2c(c(C(C)C)c1CC(=O)N=S(N)(=O)c1ccc(CN(C)C)cc1)COC2] | (R) or (S)- N-(amino(4- ((dimethylamino) methyl) phenyl)(oxo)-λ⁶- sulfaneylidene)-2-(4,6- diisopropyl-1,3- dihydroisobenzofuran- 5-yl)acetamide | Chiralpak ID, 2*25 cm, 5 um | EtOH in Hex (8 mM NH₃• MeOH) | 458 |
| 192 | 225b | 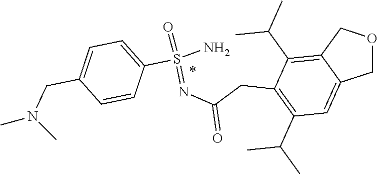 [SMILES: CC(C)c1cc2c(c(C(C)C)c1CC(=O)N=S(N)(=O)c1ccc(CN(C)C)cc1)COC2] | (S) or (R)- N-(amino(4- ((dimethylamino) methyl) phenyl)(oxo)-λ⁶- sulfaneylidene)-2-(4,6- diisopropyl-1,3- dihydroisobenzofuran- 5-yl)acetamide | Chiralpak ID, 2*25 cm, 5 um | EtOH in Hex (8 mM NH₃• MeOH) | 458 |
| 193 | 224b | 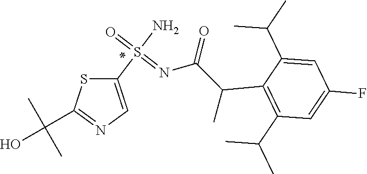 [SMILES: CC(C)c1cc(F)cc(C(C)C)c1C(C)C(=O)N=S(N)(=O)c1cnc(C(C)(C)O)s1] | (S, S) and (S, R)- N-(amino (2-(2-hydroxypropan- 2-yl)thiazol-5- yl)(oxo)-λ⁶- sulfaneylidene)-2-(4- fluoro-2,6- diisopropylphenyl) propanamide | Chiralpak IA, 2*25 cm, 5 um | IPA in Hex (0.1% FA) | 456 |
| 194 | 244ba | 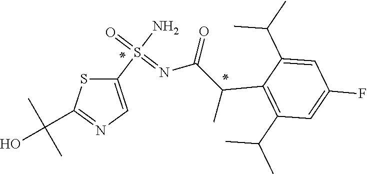 [SMILES: CC(C)c1cc(F)cc(C(C)C)c1C(C)C(=O)N=S(N)(=O)c1cnc(C(C)(C)O)s1] | (R, S) or (R, R) - N-(amino (2-(2-hydroxypropan- 2-yl)thiazol-5- yl)(oxo)-λ⁶- sulfaneylidene)-2-(4- fluoro-2,6- diisopropylphenyl) propanamide | Chiralpak IA, 2*25 cm, 5 um | IPA in Hex (0.1% FA) | 456 |
| 195 | 244aa | 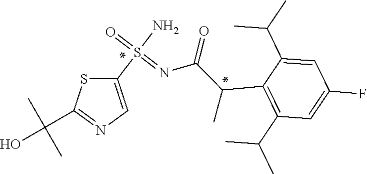 [SMILES: CC(C)c1cc(F)cc(C(C)C)c1C(C)C(=O)N=S(N)(=O)c1cnc(C(C)(C)O)s1] | (R, R) or (R, S)- N-(amino (2-(2-hydroxypropan- 2-yl)thiazol-5- yl)(oxo)-λ⁶- sulfaneylidene)-2-(4- fluoro-2,6- diisopropylphenyl) propanamide | Chiralpak IA, 2*25 cm, 5 um | IPA in Hex (0.1% FA) | 456 |
| 196 | 220a | 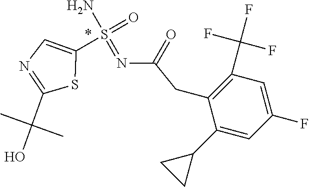 [SMILES: CC(C)(O)c1ncc(S(N)(=O)=NC(=O)Cc2c(C3CC3)cc(F)cc2C(F)(F)F)s1] | (R) or (S)-N-(amino (2-(2- hydroxypropan-2- yl)thiazol-5- yl)(oxo)-λ⁶- sulfaneylidene)-2-(2- cyclopropyl- 4-fluoro-6- (trifluoromethyl) phenyl) acetamide | Chiralpak IC, 2*25 cm, 5 um | EtOH in Hex (0.1% FA) | 466 |
| 197 | 220b | 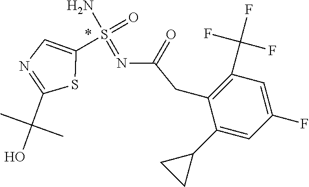 [SMILES: CC(C)(O)c1ncc(S(N)(=O)=NC(=O)Cc2c(C3CC3)cc(F)cc2C(F)(F)F)s1] | (S) or (R)-N-(amino (2-(2- hydroxypropan-2- yl)thiazol-5- yl)(oxo)-λ⁶- sulfaneylidene)-2-(2- cyclopropyl-4- fluoro-6- (trifluoromethyl) phenyl)acetamide | Chiralpak IC, 2*25 cm, 5 um | EtOH in Hex (0.1% FA) | 466 |
| 198 | 218a | 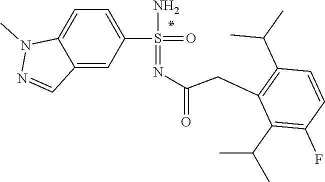 [SMILES: CC(C)c1ccc(F)c(C(C)C)c1CC(=O)N=S(N)(=O)c1ccc2c(cnn2C)c1] | (R) or (S)- N-(amino(1- methyl-1H- indazol-5- yl)(oxo)-λ⁶- sulfaneylidene)- 2-(3-fluoro-2,6- diisopropylphenyl) acetamide | CHIRAL- PAK IG, 2*25 cm (5 um) | EtOH in Hex (8 mM NH₃• MeOH) | 431 |
| 199 | 218b | 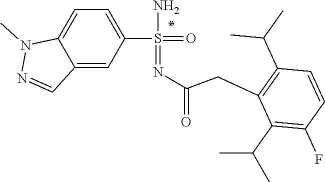 [SMILES: CC(C)c1ccc(F)c(C(C)C)c1CC(=O)N=S(N)(=O)c1ccc2c(cnn2C)c1] | (S) or (R)- N-(amino(1- methyl-1H- indazol-5- yl)(oxo)-λ⁶- sulfaneylidene)- 2-(3-fluoro-2,6- diisopropylphenyl) acetamide | CHIRAL- PAK IG, 2*25 cm (5 um) | EtOH in Hex (8 mM NH₃• MeOH) | 431 |
| 200 | 217a | 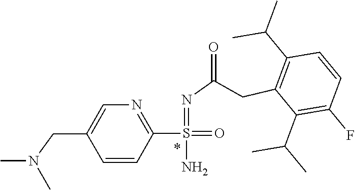 [SMILES: CC(C)c1ccc(F)c(C(C)C)c1CC(=O)N=S(N)(=O)c1ccc(CN(C)C)cn1] | (R) or (S)- N-(amino(5- ((dimethylamino) methyl) pyridin-2- yl)(oxo)-λ⁶- sulfaneylidene)-2- (3-fluoro-2,6- diisopropylphenyl) acetamide | CHIRAL- PAK ID, 2*25 cm (5 um) | IPA in Hex (8 mM NH₃• MeOH) | 435 |
| 201 | 217b | 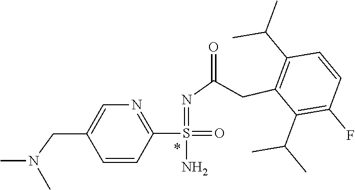 [SMILES: CC(C)c1ccc(F)c(C(C)C)c1CC(=O)N=S(N)(=O)c1ccc(CN(C)C)cn1] | (S) or (R)- N-(amino(5- ((dimethylamino) methyl) pyridin-2- yl)(oxo)-λ⁶- sulfaneylidene)-2- (3-fluoro-2,6- diisopropylphenyl) acetamide | CHIRAL- PAK ID, 2*25 cm (5 um) | IPA in Hex (8 mM NH₃• MeOH) | 435 |
| 202 | 303 | 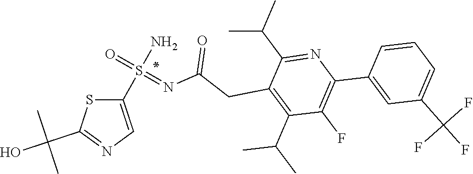 [SMILES: CC(C)c1nc(-c2cccc(C(F)(F)F)c2)c(F)c(C(C)C)c1CC(=O)N=S(N)(=O)c1cnc(C(C)(C)O)s1] | N-(amino(2-(2- hydroxypropan- 2-yl)thiazol-5- yl)(oxo)-λ⁶- sulfaneylidene)- 2-(5-fluoro-2,4- diisopropyl-6-(3- (trifluoromethyl) phenyl) pyridin-3-yl) acetamide | Chiralpak ID, 2*25 cm, 5 um | IPA in Hex (0.1% FA) | 587 |
| 203 | 302 | 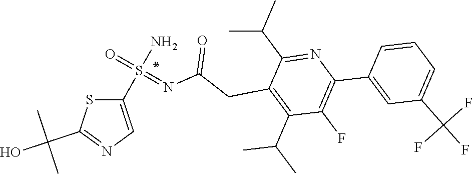 [SMILES: CC(C)c1nc(-c2cccc(C(F)(F)F)c2)c(F)c(C(C)C)c1CC(=O)N=S(N)(=O)c1cnc(C(C)(C)O)s1] | (S) or (R)-N-(amino (2-(2- hydroxypropan-2- yl)thiazol-5- yl)(oxo)-λ⁶- sulfaneylidene)-2-(5- fluoro-2,4- diisopropyl-6-(3- (trifluoromethyl) phenyl) pyridin-3-yl) acetamide | Chiralpak ID, 2*25 cm, 5 um | IPA in Hex (0.1% FA) | 587 |
| 204 | 203b | 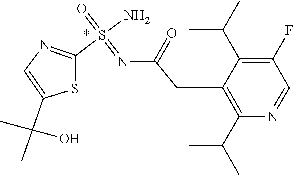 [SMILES: CC(C)c1ncc(F)c(C(C)C)c1CC(=O)N=S(N)(=O)c1ncc(C(C)(C)O)s1] | N-(amino(5- (2-hydroxypropan- 2-yl)thiazol-2- yl)(oxo)-λ⁶- sulfaneylidene)- 2-(5-fluoro-2,4- diisopropylpyridin- 3-yl)acetamide | CHIRAL- PAK IF, 2*25 cm, 5 um | EtOH in Hex (0.1% FA) | 443 |
| 205 | 203a | 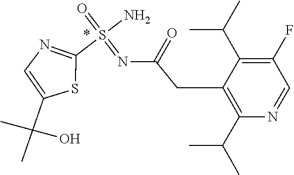 [SMILES: CC(C)c1ncc(F)c(C(C)C)c1CC(=O)N=S(N)(=O)c1ncc(C(C)(C)O)s1] | (S) or (R)- N-(amino(5- (2-hydroxypropan-2- yl)thiazol-2- yl)(oxo)-λ⁶- sulfaneylidene)-2-(5- fluoro-2,4- diisopropylpyridin- 3-yl)acetamide | CHIRAL- PAK IF, 2*25 cm, 5 um | EtOH in Hex (0.1% FA) | 443 |
| 206 | 202a | 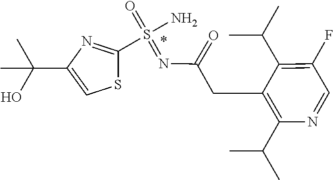 [SMILES: CC(C)c1ncc(F)c(C(C)C)c1CC(=O)N=S(N)(=O)c1nc(C(C)(C)O)cs1] | N-(amino(4-(2- hydroxypropan- 2-yl)thiazol-2- yl)(oxo)-λ⁶- sulfaneylidene)- 2-(5-fluoro-2,4- diisopropylpyridin-3- yl)acetamide | Chiralpak ID, 2*25 cm, 5 um | IPA in Hex (0.1% FA) | 443 |
| 207 | 202b | 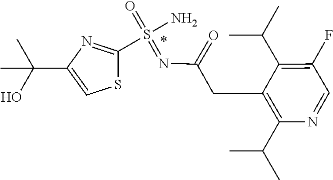 [SMILES: CC(C)c1ncc(F)c(C(C)C)c1CC(=O)N=S(N)(=O)c1nc(C(C)(C)O)cs1] | (S) or (R)- N-(amino(4- (2-hydroxypropan-2- yl)thiazol-2- yl)(oxo)-λ⁶- sulfaneylidene)-2-(5- fluoro-2,4- diisopropylpyridin- 3-yl)acetamide | Chiralpak ID, 2*25 cm, 5 um | IPA in Hex (0.1% FA) | 443 |
| 208 | 213a | 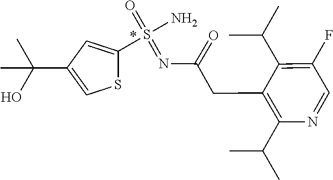 [SMILES: CC(C)c1ncc(F)c(C(C)C)c1CC(=O)N=S(N)(=O)c1cc(C(C)(C)O)cs1] | N-(amino(4-(2- hydroxypropan-2-yl) thiophen-2- yl)(oxo)-λ⁶- sulfaneylidene)- 2-(5-fluoro-2,4- diisopropylpyridin- 3-yl)acetamide | Chiralpak ID, 2*25 cm, 5 um | IPA in Hex (0.1% FA) | 442 |
| 209 | 213b | 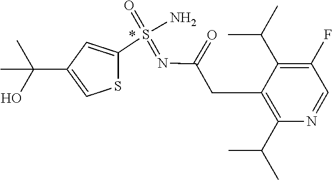 [SMILES: CC(C)c1ncc(F)c(C(C)C)c1CC(=O)N=S(N)(=O)c1cc(C(C)(C)O)cs1] | (S) or (R)- N-(amino(4- (2-hydroxypropan-2- yl)thiophen-2- yl)(oxo)-λ⁶- sulfaneylidene)-2-(5- fluoro-2,4- diisopropylpyridin- 3-yl)acetamide | Chiralpak ID, 2*25 cm, 5 um | IPA in Hex (0.1% FA) | 442 |
| 210 | 210a | 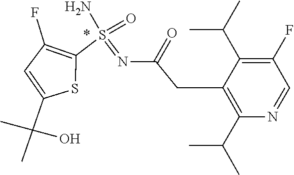 [SMILES: CC(C)c1ncc(F)c(C(C)C)c1CC(=O)N=S(N)(=O)c1sc(C(C)(C)O)cc1F] | (S) or (R)-N-(amino (3-fluoro-5-(2- hydroxypropan-2-yl) thiophen-2- yl)(oxo)-λ⁶- sulfaneylidene)- 2-(5-fluoro-2,4- diisopropylpyridin-3- yl)acetamide | CHIRAL- PAK IG, 20*250 mm, 5 um | IPA in Hex (0.1% FA) | 460 |
| 211 | 210b | 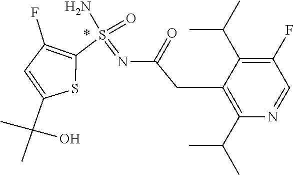 [SMILES: CC(C)c1ncc(F)c(C(C)C)c1CC(=O)N=S(N)(=O)c1sc(C(C)(C)O)cc1F] | N-(amino(3-fluoro-5- (2-hydroxypropan-2- yl)thiophen-2- yl)(oxo)-λ⁶- sulfaneylidene)-2-(5- fluoro-2,4- diisopropylpyridin-3- yl)acetamide | CHIRAL- PAK IG, 20*250 mm, 5 um | IPA in Hex (0.1% FA) | 460 |
| 212 | 216a | 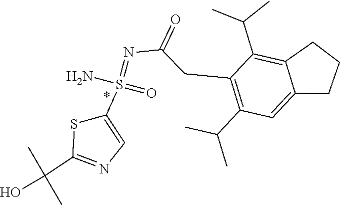 [SMILES: CC(C)c1cc2c(c(C(C)C)c1CC(=O)N=S(N)(=O)c1cnc(C(C)(C)O)s1)CCC2] | (R) or (S)-N-(amino (2-(2- hydroxypropan-2- yl)thiazol-5- yl)(oxo)-λ⁶- sulfaneylidene)- 2-(4,6- diisopropyl- 2,3-dihydro- 1H-inden-5- yl)acetamide | Chiralpak ID, 2*25 cm, 5 um | EtOH in MTBE (0.1% FA) | 464 |
| 213 | 216b | 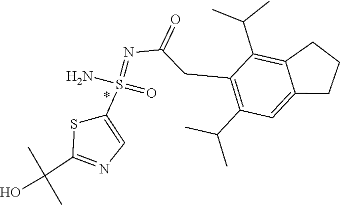 [SMILES: CC(C)c1cc2c(c(C(C)C)c1CC(=O)N=S(N)(=O)c1cnc(C(C)(C)O)s1)CCC2] | (S) or (R)- N-(amino(2- (2-hydroxypropan-2- yl)thiazol-5- yl)(oxo)-λ⁶- sulfaneylidene)- 2-(4,6- diisopropyl- 2,3-dihydro- 1H-inden-5- yl)acetamide | Chiralpak ID, 2*25 cm, 5 um | EtOH in MTBE (0.1% FA) | 464 |
| 214 | 110a′ | 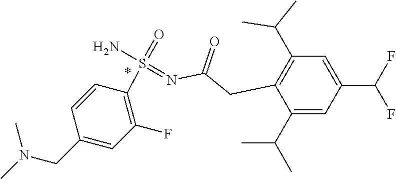 [SMILES: CC(C)c1cc(C(F)F)cc(C(C)C)c1CC(=O)N=S(N)(=O)c1ccc(CN(C)C)cc1F] | (R) or (S)- N-(amino(4- ((dimethylamino) methyl)- 2-fluorophenyl) (oxo)-λ⁶- sulfaneylidene)- 2-(4- (difluoromethyl)- 2,6- diisopropylphenyl) acetamide | CHIRAL- PAK IG, 2*25 cm (5 um) | EtOH in Hex (8 mM NH₃• MeOH) | 484 |
| 215 | 110b′ | 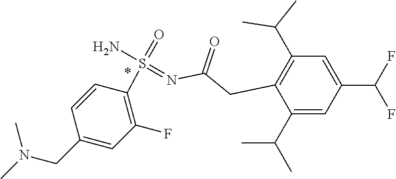 [SMILES: CC(C)c1cc(C(F)F)cc(C(C)C)c1CC(=O)N=S(N)(=O)c1ccc(CN(C)C)cc1F] | (S) or (R)- N-(amino(4- ((dimethylamino) methyl)- 2-fluorophenyl) (oxo)-λ⁶- sulfaneylidene)- 2-(4- (difluoromethyl)-2,6- diisopropylphenyl) acetamide | CHIRAL- PAK IG, 2*25 cm (5 um) | EtOH in Hex (8 mM NH₃• MeOH) | 484 |
| 216 | 263a | 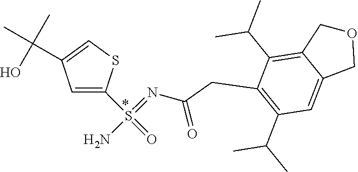 [SMILES: CC(C)c1cc2c(c(C(C)C)c1CC(=O)N=S(N)(=O)c1cc(C(C)(C)O)cs1)COC2] | (R) or (S)-N-(amino (4-(2- hydroxypropan-2- yl)thiophen-2- yl)(oxo)-λ⁶- sulfaneylidene)- 2-(4,6- diisopropyl-1,3- dihydroiso- benzofuran-5- yl)acetamide | CHIRAL- PAK IG, 2*25 cm (5 um) | EtOH in Hex (8 mM NH₃• MeOH) | 465 |
| 217 | 263b | 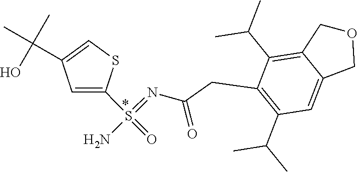 [SMILES: CC(C)c1cc2c(c(C(C)C)c1CC(=O)N=S(N)(=O)c1cc(C(C)(C)O)cs1)COC2] | (S) or (R)-N-(amino (4-(2- hydroxypropan-2- yl)thiophen-2- yl)(oxo)-λ⁶- sulfaneylidene)- 2-(4,6- diisopropyl-1,3- dihydro- isobenzofuran-5- yl)acetamide | CHIRAL- PAK IG, 2*25 cm (5 um) | EtOH in Hex (8 mM NH₃• MeOH) | 465 |
| 218 | 264a | 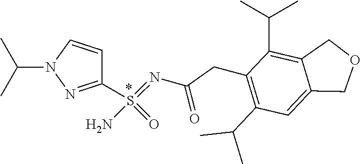 [SMILES: CC(C)c1cc2c(c(C(C)C)c1CC(=O)N=S(N)(=O)c1ccn(C(C)C)n1)COC2] | (R) or (S)- N-(amino(1- isopropyl-1H- pyrazol-3- yl)(oxo)-λ⁶- sulfaneylidene)- 2-(4,6-diisopropyl- 1,3-dihydro- isobenzofuran- 5-yl)acetamide | Chiralpak ID, 2*25 cm, 5 um | EtOH in Hex (0.1% FA) | 433 |
| 219 | 264b | 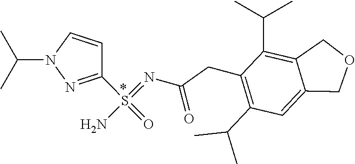 [SMILES: CC(C)c1cc2c(c(C(C)C)c1CC(=O)N=S(N)(=O)c1ccn(C(C)C)n1)COC2] | (S) or (R)- N-(amino(1- isopropyl-1H- pyrazol-3- yl)(oxo)-λ⁶- sulfaneylidene)- 2-(4,6-diisopropyl- 1,3-dihydro- isobenzofuran- 5-yl)acetamide | Chiralpak ID, 2*25 cm, 5 um | EtOH in Hex (0.1% FA) | 433 |

Examples in the following table were obtained from chiral HPLC resolutions of racemic examples described above. The chiral column and eluents are listed in the table. As a convention, the faster-eluting enantiomer is always listed first in the table followed by the slower-eluting enantiomer of the pair. The symbol * at a chiral center denotes that this chiral center has been resolved and the absolute stereochemistry at that center has not been determined. Assigned stereochemistry in compound names are tentative.

Example 159: ¹H NMR (400 MHz, DMSO-d₆) δ 7.92 (s, 2H), 7.62 (s, 1H), 7.60 (s, 1H), 7.08 (dd, J=8.7, 5.5 Hz, 1H), 6.91 (dd, J=11.9, 8.6 Hz, 1H), 5.19 (s, 1H), 3.68 (d, J=2.5 Hz, 2H), 3.20-2.90 (m, 2H), 1.39 (s, 6H), 1.25-1.04 (m, 12H).

Example 183: ¹H NMR (400 MHz, DMSO-d₆) δ 8.07 (br s, 3H), 7.04 (s, 1H), 6.29 (s, 1H), 5.05 (s, 2H), 4.88 (s, 2H), 3.77-3.62 (m, 2H), 3.25-2.90 (m, 2H), 1.48 (s, 6H), 1.15-0.99 (m, 12H).

Example 216: ¹H NMR (300 MHz, DMSO-d₆) δ 7.82 (br s, 2H), 7.59 (s, 1H), 7.58 (s, 1H), 7.03 (s, 1H), 5.19 (s, 1H), 5.04 (s, 2H), 4.87 (s, 2H), 3.67 (s, 2H), 3.25-3.00 (m, 2H), 1.38 (s, 6H), 1.20-0.80 (m, 12H).

### Example 222 (Compound 1304)

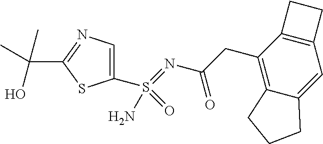 [SMILES: CC(C)(O)c1ncc(S(N)(=O)=NC(=O)Cc2c3c(cc4c2CC4)CCC3)s1]

### N-(amino(2-(2-hydroxypropan-2-yl)thiazol-5-yl)(oxo)-λ⁶-sulfaneylidene)-2-(2,4,5,6-tetrahydro-1H-cyclobuta[f]inden-3-yl)acetamide (Scheme I)

### Examples 223 (Compound 1304a) and Example 224 (Compound 1304b)

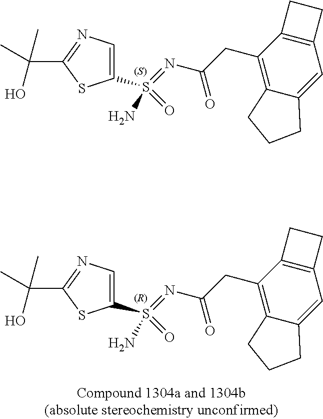

### (S)- and (R)—N-(amino(2-(2-hydroxypropan-2-yl)thiazol-5-yl)(oxo)-λ⁶-sulfaneylidene)-2-(2,4,5,6-tetrahydro-1H-cyclobuta[f]inden-3-yl)acetamide

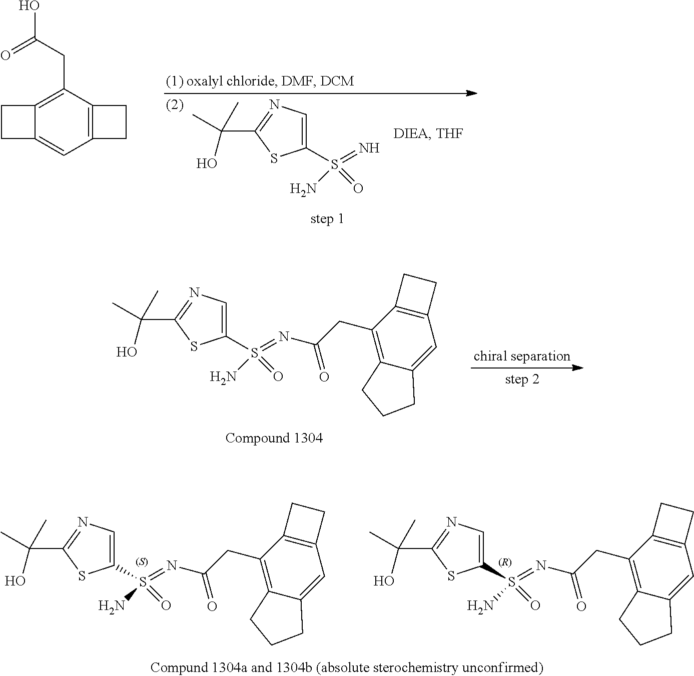

### Step 1: N-(amino(2-(2-hydroxypropan-2-yl)thiazol-5-yl)(oxo)-λ⁶-sulfaneylidene)-2-(2,4,5,6-tetrahydro-1H-cyclobuta[f]inden-3-yl)acetamide

To a stirred solution of 2-(2,4,5,6-tetrahydro-1H-cyclobuta[f]inden-3-yl)acetic acid (500 mg, 2.5 mmol) in DCM (15 mL) in a 50-mL round-bottom flask under nitrogen was added DMF (cat.) and oxalic dichloride (1.59 g, 12.5 mmol) dropwise at 0° C. The resulting solution was stirred for 30 min at RT. The resulting mixture was concentrated under vacuum; the crude product was dissolved in THF (10 mL). This solution was assigned as solution A. Into a 100-mL round-bottom flask under nitrogen, was placed a solution of 2-(2-hydroxypropan-2-yl)thiazole-5-sulfonimidamide (500 mg, 2.25 mmol) in THF (20 mL), to the stirred solution was added DIEA (585 mg, 4.55 mmol). The resulting mixture was stirred for 10 min at RT. This solution was assigned as solution B. Then to the solution B was added the solution A dropwise with stirring at 0° C. The resulting solution was stirred for 2 h at RT. The resulting mixture was concentrated under vacuum. The residue was purified by Prep-TLC to afford 175 mg crude product as a brown solid. The crude product was purified by Flash-Prep-HPLC with the following conditions: XBridge Prep OBD Cis Column 30×150 mm 5 um; Mobile Phase A: water (10 mM NH₄HCO₃), Mobile Phase B: MeCN; Flow rate: 60 mL/min; Gradient: 16% B to 40% B over 7 min; UV 254/220 nm; Rt: 7.05 min. This resulted in 253 mg (25%) of the title compound as a white solid. MS-ESI: 406 (M+1).

### Step 2: Chiral Separation

Compound 1304 (210 mg) was resolved by Prep-Chiral-HPLC with the following conditions: Column: CHIRALPAK IC, 2*25 cm, 5 um; Mobile Phase A: Hex (0.1% FA), Mobile Phase B: EtOH; Flow rate: 20 mL/min; Gradient: 15% B to 15% B in 11 min; UV 220/254 nm; Rt₁: 6.544 min (Compound 1304a); Rt₂: 8.073 min (Compound 1304b). This resulted in 61 mg of (Compound 1304a) and 70 mg of (Compound 1304b), both as white solids. Compound 1304a: MS-ESI: 406 (M+1). ¹H NMR (400 MHz, DMSO-d₆) δ 8.04 (br s, 2H), 8.03 (s, 1H), 6.79 (s, 1H), 6.29 (s, 1H), 3.43 (s, 2H), 2.98-2.86 (m, 4H), 2.85-2.75 (m, 2H), 2.75-2.65 (m, 2H), 2.05-1.85 (m, 2H), 1.50 (s, 6H). Compound 1304b, MS-ESI: 406 (M+1). ¹H NMR (400 MHz, DMSO-d₆) δ 8.10 (br s, 2H), 8.05 (s, 1H), 6.79 (s, 1H), 6.30 (s, 1H), 3.43 (s, 2H), 2.99-2.85 (m, 4H), 2.85-2.75 (m, 2H), 2.75-2.65 (m, 2H), 2.05-1.85 (m, 2H), 1.50 (s, 6H)

TABLE 27

| | | | | Exact |
|---|---|---|---|---|
| Ex. | Final | | | Mass |
| # | Target # | Structure | IUPAC Name | [M + H]⁺ |
| 225 | 1333 | 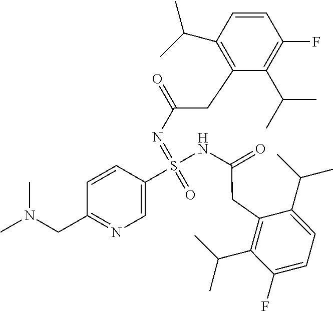 [SMILES: CC(C)c1ccc(F)c(C(C)C)c1CC(=O)N=S(=O)(NC(=O)Cc1c(C(C)C)ccc(F)c1C(C)C)c1ccc(CN(C)C)nc1] | N-(6-((dimethylamino)methyl)- N-(2-(3-fluoro-2,6- diisopropylphenyl)acetyl)pyridine- 3-sulfonimidoyl)-2-(3- fluoro-2,6-diisopropylphenyl) acetamide | 655 |

Examples in the following table were prepared using similar conditions as described in Example 222 (Compound 1304) and Scheme I from appropriate starting materials.

### Example 226 (Compound 1308)

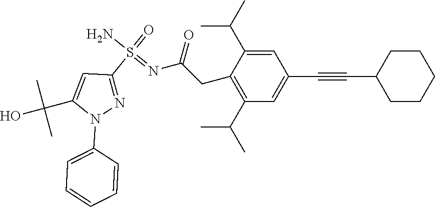 [SMILES: CC(C)c1cc(C#CC2CCCCC2)cc(C(C)C)c1CC(=O)N=S(N)(=O)c1cc(C(C)(C)O)n(-c2ccccc2)n1]

### N-(amino(5-(2-hydroxypropan-2-yl)-1-phenyl-1H-pyrazol-3-yl)(oxo)-λ⁶-sulfaneylidene)-2-(4-(cyclohexylethynyl)-2,6-diisopropylphenyl)acetamide (Scheme II)

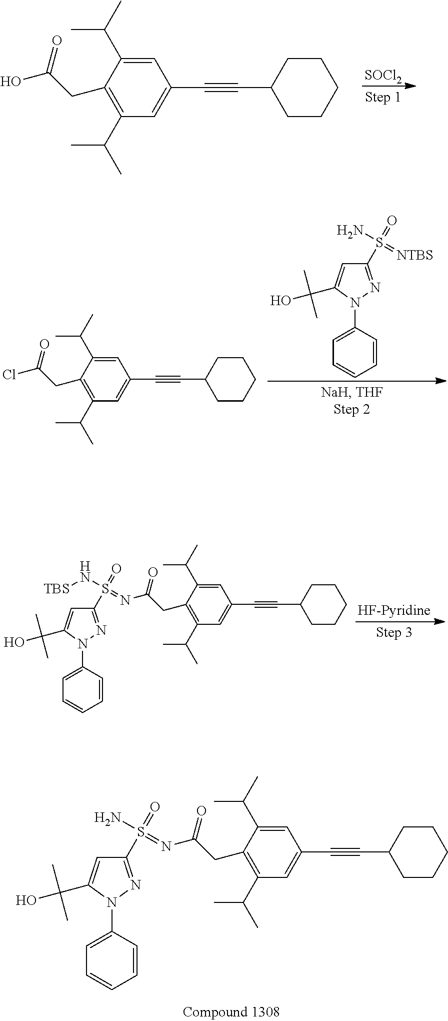

### Step 1: 2-(4-(cyclohexylethynyl)-2,6-diisopropylphenyl)acetyl Chloride

To a stirred solution of 2-(4-(cyclohexylethynyl)-2,6-diisopropylphenyl) acetic acid (200 mg, 0.61 mmol) in DCM (10 mL) in a 50-mL round-bottom flask was added SOCl₂ (363 mg, 3.05 mmol) dropwise at 0° C. The resulting solution was stirred for 1 h at RT. The resulting mixture was concentrated under vacuum. This resulted in 210 mg (crude) of the title compound as brown oil.

### Step 2: N-(((tert-butyldimethylsilyl)amino)(5-(2-hydroxypropan-2-yl)-1-phenyl-1H-pyrazol-3-yl)(oxo)-λ⁶-sulfaneylidene)-2-(4-(cyclohexylethynyl)-2,6-diisopropylphenyl)acetamide

To a stirred solution of N′-(tert-butyldimethylsilyl)-5-(2-hydroxypropan-2-yl)-1-phenyl-1H-pyrazole-3-sulfonimidamide (241 mg, 0.61 mmol) in THF (3 mL) in a 50-mL round-bottom flask under nitrogen was added NaH (60% dispersion in mineral oil, 48.8 mg, 1.22 mmol) in portions at 0° C. The resulting solution was stirred for 10 min at RT. Then 2-(4-(cyclohexylethynyl)-2,6-diisopropylphenyl)acetyl chloride (210 mg, crude) in THF (5 mL) was added to the above solution dropwise at 0° C. The resulting solution was stirred for 2 h at RT. The reaction was then quenched by the addition of 1 mL of water. The resulting mixture was concentrated under reduced pressure. The residue was eluted from silica gel with MeOH/DCM (1:10). This resulted in 250 mg (58%) of the title compound as a solid. MS-ESI: 703 (M+1).

### Step 3: N-(amino(5-(2-hydroxypropan-2-yl)-1-phenyl-1H-pyrazol-3-yl)(oxo)-λ⁶-sulfaneylidene)-2-(4-(cyclohexylethynyl)-2,6-diisopropylphenyl)acetamide

To a stirred solution of N-(((tert-butyldimethylsilyl)amino)(5-(2-hydroxypropan-2-yl)-1-phenyl-1H-pyrazol-3-yl)(oxo)-λ⁶-sulfaneylidene)-2-(4-(cyclohexylethynyl)-2,6-diisopropylphenyl) acetamide (250 mg, 0.36 mmol) in THF (10 mL) in a 50-mL round-bottom flask, was added HF-Pyridine (70% wt., 0.5 mL). The resulting solution was stirred for 2 h at RT and concentrated under vacuum. The crude product was purified by Prep-HPLC with the following conditions: XBridge Prep OBD C18 Column 30×150 mm 5 um; Mobile Phase A: water (10 mM NH₄HCO₃+0.1% NH₃.H₂O), Mobile Phase B: MeCN; Flow rate: 60 mL/min; Gradient: 52% B to 54% B over 7 min; UV 254/210 nm; Rt: 5.87 min. This resulted in 90 mg (26.9%) of the title compound as an off-white solid. MS-ESI 589 (M+1). ¹H NMR (400 MHz, DMSO-d₆) δ 7.73 (s, 2H), 7.57-7.45 (m, 5H), 7.03 (s, 2H), 6.67 (s, 1H), 5.43 (s, 1H), 3.68 (s, 2H), 3.15-2.95 (m, 2H), 2.65-2.53 (m, 1H), 1.90-1.75 (m, 2H), 1.75-1.60 (m, 2H), 1.60-1.40 (m, 3H), 1.37-1.31 (m, 3H), 1.30 (s, 6H), 1.08 (d, J=6.7 Hz, 6H), 1.04 (d, J=6.7 Hz, 6H).

TABLE 28
Example 227 (Compound 1318) was obtained during the Prep-HPLC purification of
Example 226 (Compound 1308).

| | | | | Exact |
|---|---|---|---|---|
| Ex. | Final | | | Mass |
| # | Target # | Structure | IUPAC Name | [M + H]⁺ |
| 227 | 1318 | 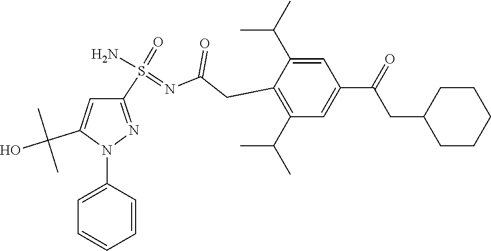 [SMILES: CC(C)c1cc(C(=O)CC2CCCCC2)cc(C(C)C)c1CC(=O)N=S(N)(=O)c1cc(C(C)(C)O)n(-c2ccccc2)n1] | N-(amino(5-(2-hydroxypropan- 2-yl)-1-phenyl-1H- pyrazol-3-yl)(oxo- sulfaneylidene)-2-(4-(2- cyclohexylacetyl)-2,6- diisopropylphenyl)acetamide | 607 |

TABLE 29
Examples in the following table were prepared using similar conditions as described in
Example 220 (Compound 1308) and Scheme II from appropriate starting materials.

| | | | | Exact |
|---|---|---|---|---|
| | Final | | | Mass |
| Ex. # | Target # | Structure | IUPAC Name | [M + H]⁺ |
| 228 | 1307 | 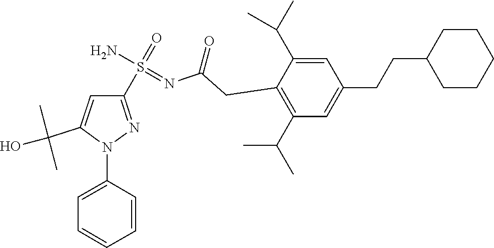 [SMILES: CC(C)c1cc(CCC2CCCCC2)cc(C(C)C)c1CC(=O)N=S(N)(=O)c1cc(C(C)(C)O)n(-c2ccccc2)n1] | N-(amino(5-(2-hydroxypropan- 2-yl)-1-phenyl-1H-pyrazol- 3-yl)(oxo)-λ⁶-sulfaneylidene)- 2-(4-(2-cyclohexylethyl)-2,6- diisopropylphenyl)acetamide | 593 |
| 229 | 1319 | 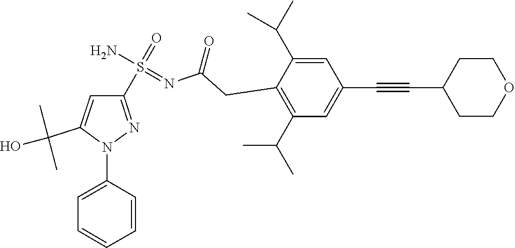 [SMILES: CC(C)c1cc(C#CC2CCOCC2)cc(C(C)C)c1CC(=O)N=S(N)(=O)c1cc(C(C)(C)O)n(-c2ccccc2)n1] | N-(amino(5-(2-hydroxypropan- 2-yl)-1-phenyl-1H-pyrazol- 3-yl)(oxo)-λ⁶-sulfaneylidene)- 2-(2,6-diisopropyl-4- ((tetrahydro-2H-pyran-4-yl) ethynyl)phenyl)acetamide | 591 |
| 230 | 1332 | 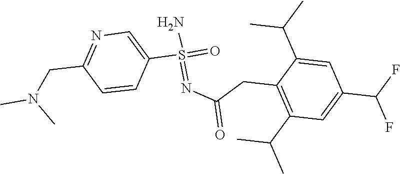 [SMILES: CC(C)c1cc(C(F)F)cc(C(C)C)c1CC(=O)N=S(N)(=O)c1ccc(CN(C)C)nc1] | N-(amino(6-((dimethylamino) methyl)pyridin-3-yl)(oxo)-λ⁶- sulfaneylidene)-2-(4- (difluoromethyl)-2,6- diisopropylphenyl)acetamide | 467 |

### Example 231 (Compound 1336a)

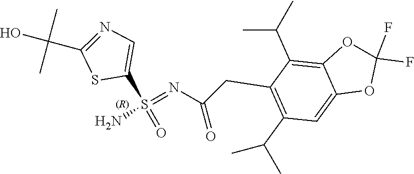

### (R)—N-amino(2-(2-hydroxypropan-2-yl)thiazol-5-yl)(oxo)-λ⁶-sulfaneylidene)-2-(2,2-difluoro-4,6-diisopropylbenzo[d][1,3]dioxol-5-yl)acetamide (Scheme III)

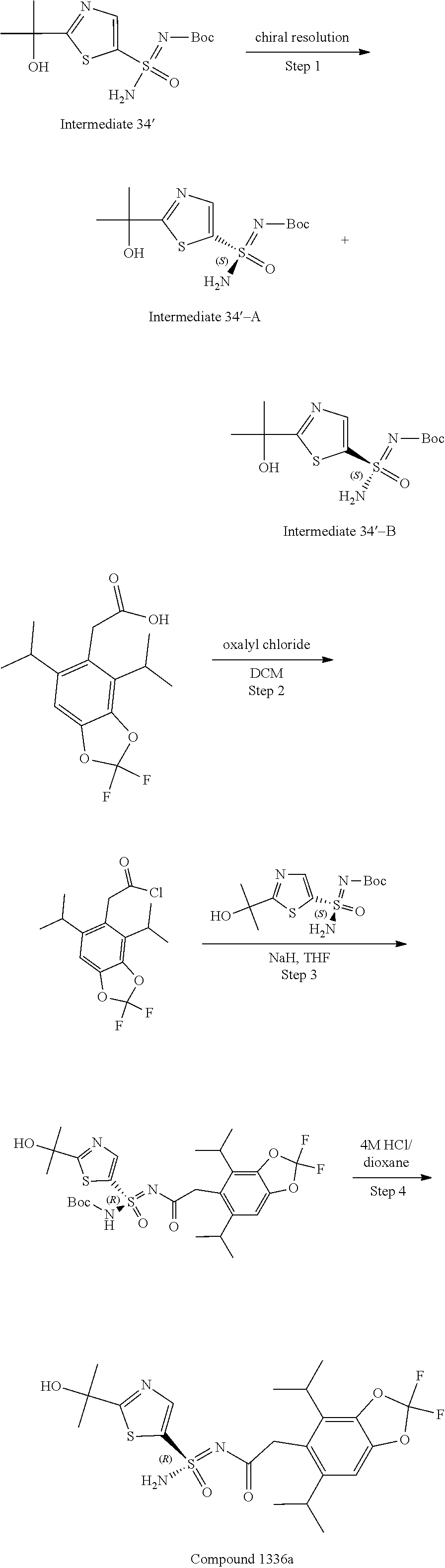

### Step 1: Chiral Resolution (R) and (S)-tert-butyl(amino(2-(2-hydroxypropan-2-yl)thiazol-5-yl)(oxo)-λ⁶-sulfaneylidene)carbamate

The product (10 g intermediate 34) was separated with the followed condition: Column: CHIRALPAK IC, 5*25 cm, 5 um; Mobile Phase A: 50% CO₂, Mobile Phase B: 50% EtOH:Hex=1:1; Flow rate: 150 mL/min; UV 220 nm; Rt₁: 5.13 min (Intermediate 34A); Rt₂: 5.65 min. (Intermediate 34B). This resulted in 3 g (99.5% ee) of Intermediate 34′-A and 3 g (99.0% ee) of Intermediate 28′-B. The absolute stereochemistry of Intermediates 34′-A and 34′-B were both determined by single crystal X-ray crystallography.

### Step 2: 2-(2,2-Difluoro-4,6-diisopropylbenzo[d][1,3]dioxol-5-yl)acetyl Chloride

To a stirred solution of 2-(2,2-difluoro-4,6-diisopropylbenzo[d][1,3]dioxol-5-yl)acetic acid (234 mg, 0.78 mmol) in DCM (8 mL) in a 50-mL round-bottom flask was added DMF (cat.) and oxalyl dichloride (0.70 mL) at 0° C. The resulting solution was stirred for 1 h at RT. The resulting mixture was concentrated under vacuum. This resulted in 258 mg (crude) of the title compound as brown oil.

### Step 3: Tert-butyl (R)—(N-(2-(2,2-difluoro-4,6-diisopropylbenzo[d][1,3]dioxol-5-yl)acetyl)-2-(2-hydroxypropan-2-yl)thiazole-5-sulfonimidoyl)carbamate

To a stirred solution of tert-butyl (S)-(amino(2-(2-hydroxypropan-2-yl)thiazol-5-yl)(oxo)-λ⁶-)carbamate (252 mg, 0.78 mmol) in THF (5 mL) in a 50-mL round-bottom flask under nitrogen was added NaH (60% dispersion in mineral oil, 37.6 mg, 1.57 mmol) in portions at 0° C. The resulting solution was stirred for 10 min at RT. Then 2-(2,2-difluoro-4,6-diisopropylbenzo[d][1,3]dioxol-5-yl)acetyl chloride (258 mg, crude) in THF (5 mL) was added to the above solution dropwise at 0° C. The resulting solution was stirred for 2 h at RT. The reaction was then quenched by the addition of 1 mL of water. The resulting mixture was concentrated under vacuum. The residue was eluted from silica gel with MeOH/DCM (1:10). This resulted in 250 mg (52.8%) of the title compound as a solid. MS-ESI: 604 (M+1).

### Step 4: (R)—N-(amino(2-(2-hydroxypropan-2-yl)thiazol-5-yl)(oxo)-λ⁶-sulfaneylidene)-2-(2,2-difluoro-4,6-diisopropylbenzo[d][1,3]dioxol-5-yl)acetamide

To a stirred solution of tert-butyl (R)—(N-(2-(2,2-difluoro-4,6-diisopropylbenzo[d][1,3]dioxol-5-yl)acetyl)-2-(2-hydroxypropan-2-yl)thiazole-5-sulfonimidoyl)carbamate (300 mg, 0.5 mmol) in 4 M HCl in 1,4-dioxane (5 mL) a 50-mL round-bottom flask. The resulting solution was stirred for 2 h at RT. The resulting mixture was concentrated under vacuum. The crude product was purified by Prep-HPLC with the following conditions: XBridge Prep C18 OBD Column 19×150 mm 5 um; Mobile Phase A: water (10 mM NH₄HCO₃), Mobile Phase B: MeCN; Flow rate: 60 mL/min; Gradient: 34% B to 64% B in 7 min; UV 254 nm; Rt: 6.07 min. This resulted in 188 mg (62%) of the title compound as a white solid. MS-ESI: 504 (M+1). ¹H NMR (300 MHz, DMSO-d₆) δ 8.09 (s, 1H), 8.08 (br s, 2H), 7.14 (s, 1H), 6.29 (s, 1H), 3.80-3.60 (m, 2H), 3.20-3.00 (m, 2H), 1.49 (s, 6H), 1.20 (d, J=6.9 Hz, 3H), 1.18-0.95 (m, 9H).

TABLE 30
Examples in the following table were prepared using similar conditions as described in
Example 231 (Compound 1336a) and Scheme III from appropriate starting materials.

| | | | | Exact |
|---|---|---|---|---|
| Ex. | Final | | | Mass |
| # | Target # | Structure | IUPAC Name | [M + H]⁺ |
| 232 | 1302b | 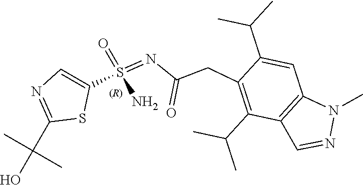 | (R)-N-(amino(2-(2-hydroxypropan-2-yl)thiazol-5- yl)(oxo)-λ⁶-sulfaneylidene)-2-(4,6-diisopropyl-1- methyl-1H-indazol-5-yl)acetamide | 478 |
| 233 | 1342a | 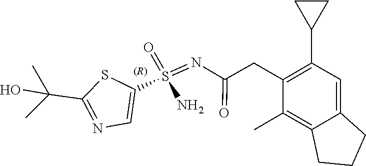 | (R)-N-(amino(2-(2-hydroxypropan-2-yl)thiazol-5- yl)(oxo)-λ⁶-sulfaneylidene)-2-(6-cyclopropyl-4- methyl-2,3-dihydro-1H-inden-5-yl)acetamide | 434 |

### Example 234 (Compound 1337a)

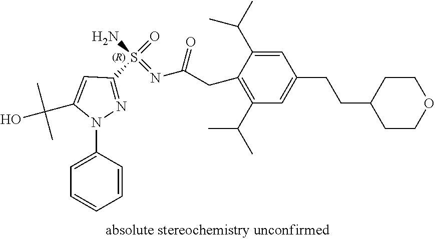

### (R) or (S)—N-(amino(5-(2-hydroxypropan-2-yl)-1-phenyl-1H-pyrazol-3-yl)(oxo)-λ⁶-sulfaneylidene)-2-(2,6-diisopropyl-4-(2-(tetrahydro-2H-pyran-4-yl)ethyl)phenyl)acetamide (Scheme IV)

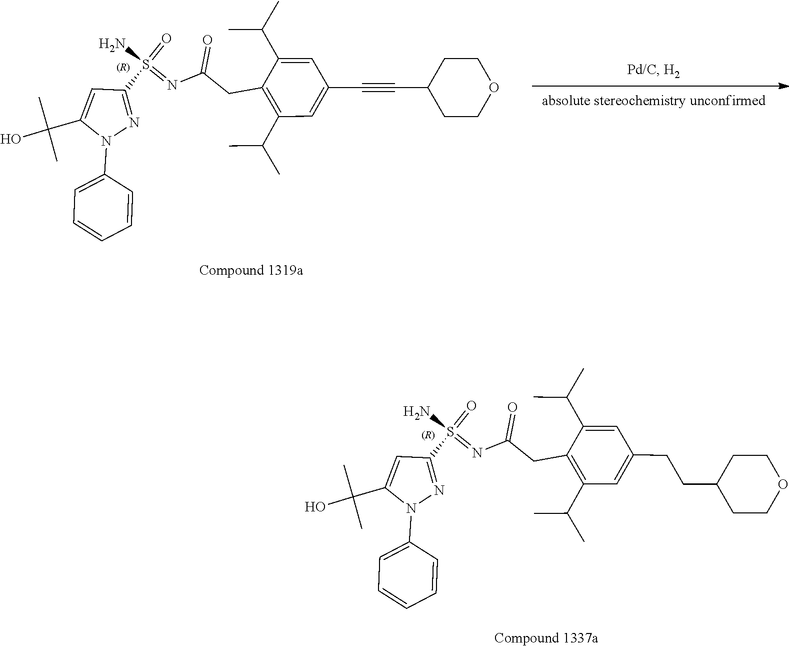

### Step 1: (R) or (S)—N-(amino(5-(2-hydroxypropan-2-yl)-1-phenyl-1H-pyrazol-3-yl)(oxo)-λ⁶-sulfaneylidene)-2-(2,6-diisopropyl-4-(2-(tetrahydro-2H-pyran-4-yl)ethyl)phenyl)acetamide

To a stirred solution (R) or (S)—N-(amino(5-(2-hydroxypropan-2-yl)-1-phenyl-1H-pyrazol-3-yl)(oxo)-λ⁶-sulfaneylidene)-2-(2,6-diisopropyl-4-((tetrahydro-2H-pyran-4-yl)ethynyl)phenyl)acetamide (Compound 1319a) (40 mg, 0.068 mmol) in MeOH (5 mL) in a 50-mL round-bottom flask was added Pd/C (10% wt., 5.0 mg). The flask was evacuated and refilled hydrogen with a balloon. The mixture was hydrogenated at RT for 6 h under hydrogen using a balloon. The solids were filtered out. The resulting mixture was concentrated under vacuum. The crude product was purified by Prep-HPLC with the following conditions: XBridge Prep OBD C18 Column 30×150 mm 5 um; Mobile Phase A: water (10 mM NH₄HCO₃+0.1% NH₃.H₂O), Mobile Phase B: MeCN; Flow rate: 60 mL/min; Gradient: 33% B to 55% B in 7 min; UV 254/210 nm; Rt: 6.50 min. This resulted in 30.8 mg (76.5%) of the title compound as an off-white solid. MS-ESL: 595 (M+1).

TABLE 31
Example 235 was obtained from Example 278 (Compound 1319b) using similar
procedure for converting Example 277 (Compound 1319a) to Example 234.

| | | | | Exact |
|---|---|---|---|---|
| Ex. | Final | | | Mass |
| # | Target # | Structure | IUPAC Name | [M + H]⁺ |
| 235 | 1337b | 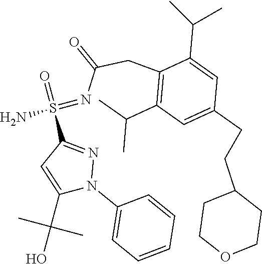 [SMILES: CC(C)c1cc(CCC2CCOCC2)cc(C(C)C)c1CC(=O)N=[S@](N)(=O)c1cc(C(C)(C)O)n(-c2ccccc2)n1] | (S) or (R)-N-(amino(5-(2-hydroxypropan-2-yl)-1-phenyl- 1H-pyrazol-3-yl)(oxo)-λ⁶-2-(2,6-diisopropyl-4-(2- (tetrahydro-2H-pyran-4-yl)ethyl)phenyl)acetamide | 595 |

TABLE 32
Examples in the following table were prepared using similar conditions as described in
Example 88 and Scheme 1A from appropriate starting materials.

| | Final | | | Exact |
|---|---|---|---|---|
| Ex. | Target | | | Mass |
| # | # | Structure | IUPAC Name | [M + H]⁺ |
| 236 | 1330 | 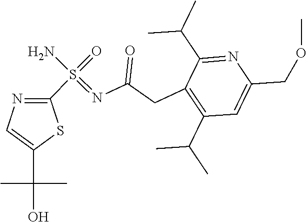 [SMILES: COCc1cc(C(C)C)c(CC(=O)N=S(N)(=O)c2ncc(C(C)(C)O)s2)c(C(C)C)n1] | N-(amino(5-(2-hydroxypropan-2-yl)thiazol- 2-yl)(oxo)-λ⁶-sulfaneylidene)-2-(2,4- diisopropyl-6-(methoxymethyl)pyridin-3- yl)acetamide | 469 |
| 237 | 1334 | 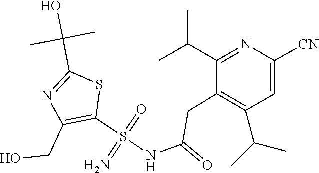 [SMILES: CC(C)c1cc(C#N)nc(C(C)C)c1CC(=O)NS(=N)(=O)c1sc(C(C)(C)O)nc1CO] | 2-(6-cyano-2,4-diisopropylpyridin-3-yl)-N- (4-(hydroxymethyl)-2-(2-hydroxypropan- 2-yl)thiazole-5-sulfonimidoyl)acetamide | 480 |
| 238 | 1315 | 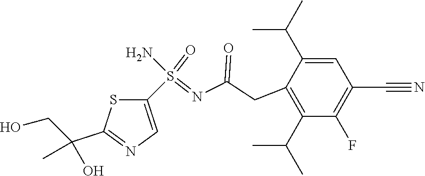 [SMILES: CC(C)c1cc(C#N)c(F)c(C(C)C)c1CC(=O)N=S(N)(=O)c1cnc(C(C)(O)CO)s1] | N-(amino(2-(1,2-dihydroxypropan-2-yl) thiazol-5-yl)(oxo)-λ⁶-sulfaneylidene)-2-(4- cyano-3-fluoro-2,6-diisopropylphenyl) acetamide | 483 |
| 239 | 1324 | 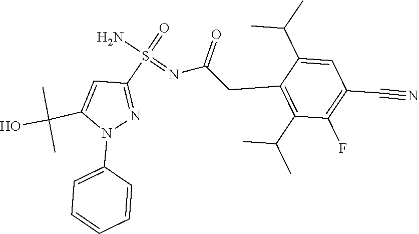 [SMILES: CC(C)c1cc(C#N)c(F)c(C(C)C)c1CC(=O)N=S(N)(=O)c1cc(C(C)(C)O)n(-c2ccccc2)n1] | N-(amino(5-(2-hydroxypropan-2-yl)-1- phenyl-1H-pyrazol-3-yl)(oxo)-λ⁶- sulfaneylidene)-2-(4-cyano-3-fluoro-2,6- diisopropylphenyl)acetamide | 526 |
| 240 | 1314 | 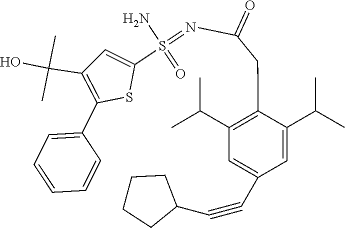 [SMILES: CC(C)c1cc(C#CC2CCCC2)cc(C(C)C)c1CC(=O)N=S(N)(=O)c1cc(C(C)(C)O)c(-c2ccccc2)s1] | N-(amino(4-(2-hydroxypropan-2-yl)-5- phenylthiophen-2-yl)(oxo)-λ⁶- sulfaneylidene)-2-(4-(cyclopentylethynyl)- 2,6-diisopropylphenyl)acetamide | 591 |
| 241 | 1313 | 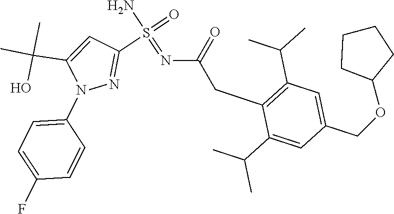 [SMILES: CC(C)c1cc(COC2CCCC2)cc(C(C)C)c1CC(=O)N=S(N)(=O)c1cc(C(C)(C)O)n(-c2ccc(F)cc2)n1] | N-(amino(1-(4-fluorophenyl)-5-(2- hydroxypropan-2-yl)-1H-pyrazol-3-yl)(oxo)- λ⁶-sulfaneylidene)-2-(4-((cyclopentyloxy) methyl)-2,6-diisopropylphenyl)acetamide | 599 |
| 242 | 1335 | 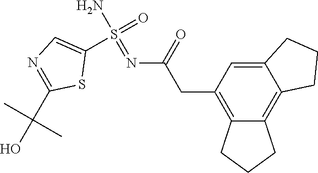 [SMILES: CC(C)(O)c1ncc(S(N)(=O)=NC(=O)Cc2cc3c(c4c2CCC4)CCC3)s1] | N-(amino(2-(2-hydroxypropan-2-yl)thiazol-5- yl)(oxo)-λ⁶-sulfaneylidene)-2-(1,2,3,6,7,8- hexahydro-as-indacen-4-yl)acetamide | 420 |
| 243 | 1328 | 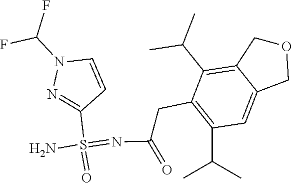 [SMILES: CC(C)c1cc2c(c(C(C)C)c1CC(=O)N=S(N)(=O)c1ccn(C(F)F)n1)COC2] | N-(amino(1-(difluoromethyl)-1H-pyrazol-3- yl)(oxo)-λ⁶-sulfaneylidene)-2-(4,6- diisopropyl-1,3-dihydroisobenzofuran-5-yl) acetamide | 441 |
| 244 | 1321 | 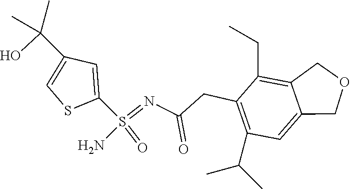 [SMILES: CCc1c2c(cc(C(C)C)c1CC(=O)N=S(N)(=O)c1cc(C(C)(C)O)cs1)COC2] | N-(amino (4-(2-hydroxypropan-2-yl)thiophen- 2-yl)(oxo)-λ⁶-sulfaneylidene)-2-(4-ethyl-6- isopropyl-1,3-dihydroisobenzofuran-5-yl) acetamide | 451 |
| 245 | 1320 | 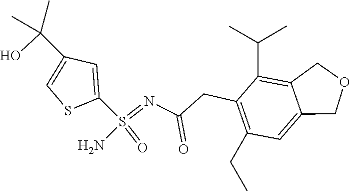 [SMILES: CCc1cc2c(c(C(C)C)c1CC(=O)N=S(N)(=O)c1cc(C(C)(C)O)cs1)COC2] | N-(amino (4-(2-hydroxypropan-2-yl)thiophen- 2-yl)(oxo)-λ⁶-sulfaneylidene)-2-(6-ethyl-4- isopropyl-1,3-dihydroisobenzofuran-5-yl) acetamide | 451 |
| 246 | 265 | 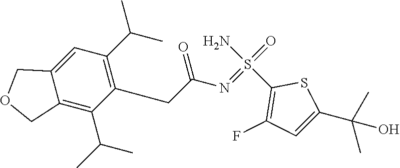 [SMILES: CC(C)c1cc2c(c(C(C)C)c1CC(=O)N=S(N)(=O)c1sc(C(C)(C)O)cc1F)COC2] | N-(amino(3-fluoro-5-(2-hydroxypropan-2-yl) thiophen-2-yl)(oxo)-λ⁶-sulfaneylidene)-2-(4,6- diisopropyl-1,3-dihydroisobenzofuran-5-yl) acetamide | 483 |
| 247 | 1303 | 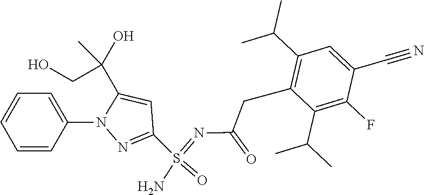 [SMILES: CC(C)c1cc(C#N)c(F)c(C(C)C)c1CC(=O)N=S(N)(=O)c1cc(C(C)(O)CO)n(-c2ccccc2)n1] | N-(amino(5-(1,2-dihydroxypropan-2-yl)-1- phenyl-1H-pyrazol-3-yl)(oxo)-λ⁶- sulfaneylidene)-2-(4-cyano-3-fluoro- 2,6-diisopropylphenyl)acetamide | 542 |
| 248 | 1327 | 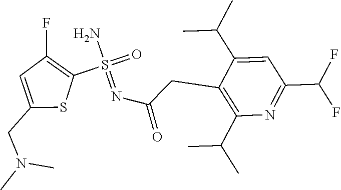 [SMILES: CC(C)c1cc(C(F)F)nc(C(C)C)c1CC(=O)N=S(N)(=O)c1sc(CN(C)C)cc1F] | N-(amino(5-((dimethylamino)methyl)-3- fluorothiophen-2-yl)(oxo)-λ⁶-sulfaneylidene)- 2-(6-(difluoromethyl)-2,4- diisopropylpyridin-3-yl)acetamide | 491 |
| 249 | 1310 | 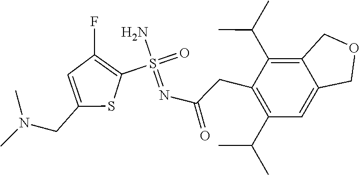 [SMILES: CC(C)c1cc2c(c(C(C)C)c1CC(=O)N=S(N)(=O)c1sc(CN(C)C)cc1F)COC2] | N-(amino(5-((dimethylamino)methyl)-3- fluorothiophen-2-yl)(oxo)-λ⁶-sulfaneylidene)- 2-(4,6-diisopropyl-1,3-dihydroisobenzofuran- 5-yl)acetamide | 482 |
| 250 | 1301 | 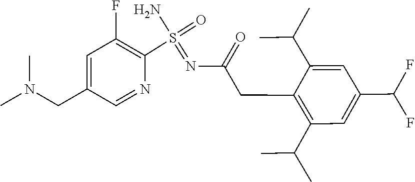 [SMILES: CC(C)c1cc(C(F)F)cc(C(C)C)c1CC(=O)N=S(N)(=O)c1ncc(CN(C)C)cc1F] | N-(amino(5-((dimethylamino)methyl)-3- fluoropyridin-2-yl)(oxo)-λ⁶-2-(4- (difluoromethyl)-2,6- diisopropylphenyl)acetamide | 485 |
| 251 | 1305 | 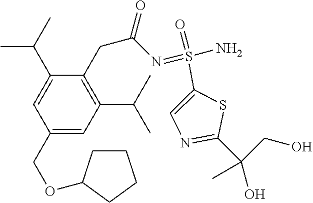 [SMILES: CC(C)c1cc(COC2CCCC2)cc(C(C)C)c1CC(=O)N=S(N)(=O)c1cnc(C(C)(O)CO)s1] | N-(amino(2-(1,2-dihydroxypropan-2-yl) thiazol-5-yl)(oxo)-λ⁶-sulfaneylidene)-2- (4-((cyclopentyloxy)methyl)-2,6- diisopropylphenyl)acetamide | 538 |

TABLE 33
Examples in the following table were prepared using similar conditions as described in
Example 93 and Scheme 1B from appropriate starting materials.

| | Final | | | Exact |
|---|---|---|---|---|
| | Target | | | Mass |
| Ex. # | # | Structure | IUPAC Name | [M + H]⁺ |
| 252 | 1312 | 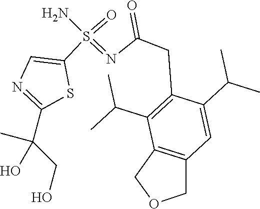 [SMILES: CC(C)c1cc2c(c(C(C)C)c1CC(=O)N=S(N)(=O)c1cnc(C(C)(O)CO)s1)COC2] | N-(amino(2-(1,2-dihydroxypropan-2-yl)thiazol-5-yl) (oxo)-λ⁶-sulfaneylidene)-2-(4,6-diisopropyl-1,3- dihydroisobenzofuran-5-yl)acetamide | 482 |
| 253 | 1309 | 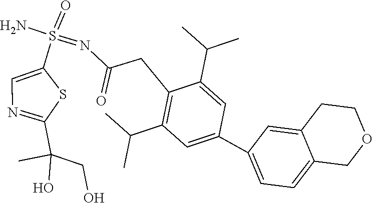 [SMILES: CC(C)c1cc(-c2ccc3c(c2)CCOC3)cc(C(C)C)c1CC(=O)N=S(N)(=O)c1cnc(C(C)(O)CO)s1] | N-(amino(2-(1,2-dihydroxypropan-2-yl)thiazol-5-yl) (oxo)-λ⁶-sulfaneylidene)-2-(4-(isochroman-6-yl)-2,6- diisopropylphenyl)acetamide | 572 |
| 254 | 1326 | 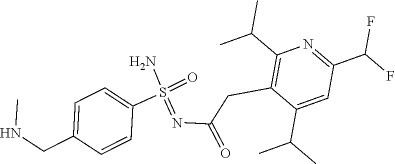 [SMILES: CNCc1ccc(S(N)(=O)=NC(=O)Cc2c(C(C)C)cc(C(F)F)nc2C(C)C)cc1] | N-(amino(4-((methylamino)methyl)phenyl)(oxo)-λ⁶- sulfaneylidene)-2-(6-(difluoromethyl)-2,4- diisopropylpyridin-3-yl)acetamide | 453 |
| 255 | 1325 | 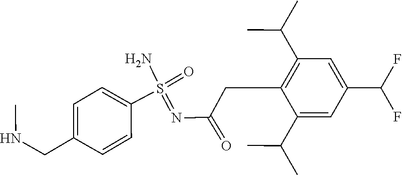 [SMILES: CNCc1ccc(S(N)(=O)=NC(=O)Cc2c(C(C)C)cc(C(F)F)cc2C(C)C)cc1] | N-(amino(4-((methylamino)methyl)phenyl)(oxo)-λ⁶- sulfaneylidene)-2-(4-(difluoromethyl)-2,6- diisopropylphenyl)acetamide | 452 |

TABLE 34
Examples in the following table were prepared using similar conditions as described in
Example 96 and Scheme 1C from appropriate starting materials.

| | Final | | | Exact |
|---|---|---|---|---|
| | Target | | | Mass |
| Ex. # | # | Structure | IUPAC Name | [M + H]⁺ |
| 256 | 1331 | 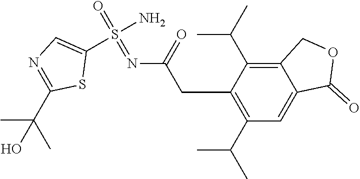 [SMILES: CC(C)c1cc2c(c(C(C)C)c1CC(=O)N=S(N)(=O)c1cnc(C(C)(C)O)s1)COC2=O] | N-(amino(2-(2-hydroxypropan-2-yl)thiazol-5-yl) (oxo)-λ⁶-sulfaneylidene)-2-(4,6-diisopropyl-1- oxo-1,3-dihydroisobenzofuran-5-yl)acetamide | 480 |
| 257 | 1338 | 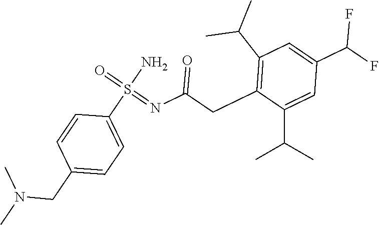 [SMILES: CC(C)c1cc(C(F)F)cc(C(C)C)c1CC(=O)N=S(N)(=O)c1ccc(CN(C)C)cc1] | N-(amino(4-((dimethylamino)methyl)phenyl) (oxo)-λ⁶-sulfaneylidene)-2-(4-(difluoromethyl)- 2,6-diisopropylphenyl)acetamide | 466 |
| 258 | 1329 | 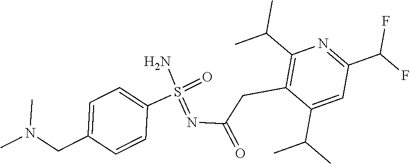 [SMILES: CC(C)c1cc(C(F)F)nc(C(C)C)c1CC(=O)N=S(N)(=O)c1ccc(CN(C)C)cc1] | N-(amino(4-((dimethylamino)methyl)phenyl) (oxo)-λ⁶-sulfaneylidene)-2-(6-(difluoromethyl)- 2,4-diisopropylpyridin-3-yl)acetamide | 467 |

TABLE 35
Examples in the following table were prepared using similar conditions as described in
Example 4 and Scheme 2 from appropriate starting materials.

| | Final | | | Exact |
|---|---|---|---|---|
| Ex. | Target | | | Mass |
| # | # | Structure | IUPAC Name | [M + H]⁺ |
| 259 | 1323 | 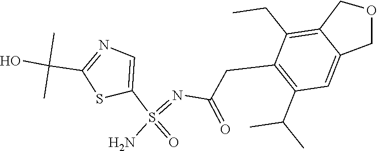 [SMILES: CCc1c2c(cc(C(C)C)c1CC(=O)N=S(N)(=O)c1cnc(C(C)(C)O)s1)COC2] | N-(amino(2-(2-hydroxypropan-2-yl)thiazol-5-yl)(oxo)- λ⁶-sulfaneylidene)-2-(4-ethyl-6-isopropyl-1,3- dihydroisobenzofuran-5-yl)acetamide | 452 |
| 260 | 1322 | 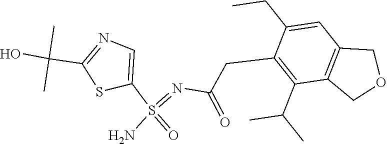 [SMILES: CCc1cc2c(c(C(C)C)c1CC(=O)N=S(N)(=O)c1cnc(C(C)(C)O)s1)COC2] | N-(amino(2-(2-hydroxypropan-2-yl)thiazol-5-yl)(oxo)- λ⁶-sulfaneylidene)-2-(6-ethyl-4-isopropyl-1,3- dihydroisobenzofuran-5-yl)acetamide | 452 |
| 262 | 1302 | 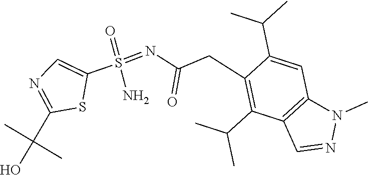 [SMILES: CC(C)c1cc2c(cnn2C)c(C(C)C)c1CC(=O)N=S(N)(=O)c1cnc(C(C)(C)O)s1] | N-(amino(2-(2-hydroxypropan-2-yl)thiazol-5-yl)(oxo)- λ⁶-sulfaneylidene)-2-(4,6-diisopropyl-1-methyl-1H- indazol-5-yl)acetamide | 478 |
| 263 | 1317 | 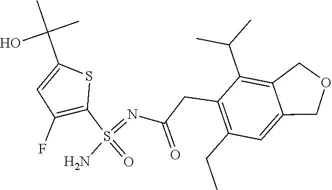 [SMILES: CCc1cc2c(c(C(C)C)c1CC(=O)N=S(N)(=O)c1sc(C(C)(C)O)cc1F)COC2] | N-(amino(3-fluoro-5-(2-hydroxypropan-2-yl)thiophen-2- yl)(oxo)-λ⁶-sulfaneylidene)-2-(6-ethyl-4-isopropyl-1,3- dihydroisobenzofuran-5-yl)acetamide | 469 |
| 264 | 1316 | 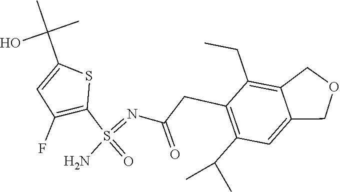 [SMILES: CCc1c2c(cc(C(C)C)c1CC(=O)N=S(N)(=O)c1sc(C(C)(C)O)cc1F)COC2] | N-(amino(3-fluoro-5-(2-hydroxypropan-2-yl)thiophen-2- yl)(oxo)-λ⁶-sulfaneylidene)-2-(4-ethyl-6-isopropyl-1,3- dihydroisobenzofuran-5-yl)acetamide | 469 |
| 265 | 1311 | 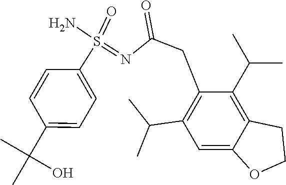 [SMILES: CC(C)c1cc2c(c(C(C)C)c1CC(=O)N=S(N)(=O)c1ccc(C(C)(C)O)cc1)CCO2] | N-(amino(4-(2-hydroxypropan-2-yl)phenyl)(oxo)-λ⁶- sulfaneylidene)-2-(4,6-diisopropyl-1,3- dihydroisobenzofuran-5-yl)acetamide | 459 |
| 266 | 1306 | 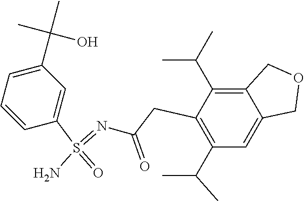 [SMILES: CC(C)c1cc2c(c(C(C)C)c1CC(=O)N=S(N)(=O)c1cccc(C(C)(C)O)c1)COC2] | N-(amino (3-(2-hydroxypropan-2-yl)phenyl)(oxo)-λ⁶- sulfaneylidene)-2-(4,6-diisopropyl-1,3- dihydroisobenzofuran-5-yl)acetamide | 459 |
| 267 | 501 | 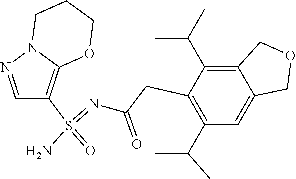 [SMILES: CC(C)c1cc2c(c(C(C)C)c1CC(=O)N=S(N)(=O)c1cnn3c1OCCC3)COC2] | N-(amino(6,7-dihydro-5H-pyrazolo[5,1-b][1,3]oxazin-3- yl)(oxo)-λ⁶-sulfaneylidene)-2-(4,6-diisopropyl-1,3- dihydroisobenzofuran-5-yl)acetamide | 447 |
| 268 | 504 | 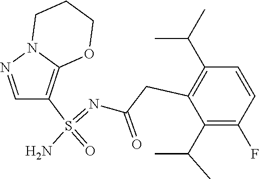 [SMILES: CC(C)c1ccc(F)c(C(C)C)c1CC(=O)N=S(N)(=O)c1cnn2c1OCCC2] | N-(amino(6,7-dihydro-5H-pyrazolo[5,1b][1,3]oxazin-3- yl)(oxo)-λ⁶-sulfaneylidene)-2-(3-fluoro-2,6- diisopropylphenyl)acetamide | 423 |

TABLE 36
The corresponding racemate of Example 260 and Example 270 in the following table
was prepared using similar conditions as described in Example 4 and Scheme 2 from appropriate
starting materials, then used chiral HPLC purification to obtain Example 269 (Compound 509b)
and Example 270 (Compound 509a) on a CHIRAL ART Cellulose-SB column (2 * 25 cm, 5 um)
eluted with 30% EtOH in Hex (0.1% FA). Compound 509b eluted first on this column followed
by Compound 509a.

| | | | | Exact |
|---|---|---|---|---|
| | Final | | | Mass |
| Ex. # | Target # | Structure | IUPAC Name | [M + H]⁺ |
| 269 | 509b | 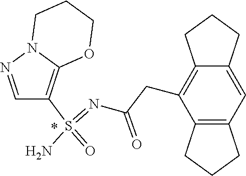 [SMILES: NS(=O)(=NC(=O)Cc1c2c(cc3c1CCC3)CCC2)c1cnn2c1OCCC2] | (R) or (S)-N-(amino(6,7-dihydro-5H-pyrazolo[5,1-b][1,3]oxazin- 3-yl)(oxo)-λ⁶-sulfaneylidene)-2-(1,2,3,5,6,7-hexahydro-s-indacen- 4-yl)acetamide | 401 |
| 270 | 509a | 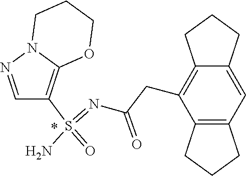 [SMILES: NS(=O)(=NC(=O)Cc1c2c(cc3c1CCC3)CCC2)c1cnn2c1OCCC2] | (S) or (R)-N-(amino(6,7-dihydro-5H-pyrazolo[5,1-b][1,3]oxazin- 3-yl)(oxo)-λ⁶-sulfaneylidene)-2-(1,2,3,5,6,7-hexahydro-s-indacen- 4-yl)acetamide | 401 |

TABLE 37
Examples in the following table were obtained from chiral HPLC resolutions of racemic
examples described above. The chiral column and eluents are listed in the table. As a convention,
the faster-eluting enantiomer is always listed first in the table followed by the slower-eluting
enantiomer of the pair. The symbol * at a chiral center denotes that this chiral center has been
resolved and the absolute stereochemistry at that center has not been determined. Assigned
stereochemistry in compound names are tentative.

| | | | | | | LC- |
|---|---|---|---|---|---|---|
| | Final | | | | | MS |
| Ex. | Target | | | | | [M + |
| # | # | Structure | IUPAC Name | Column | Eluents | H]⁺ |
| 271 | 222a | 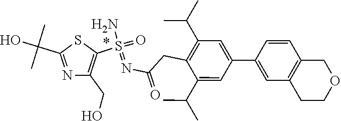 [SMILES: CC(C)c1cc(-c2ccc3c(c2)CCOC3)cc(C(C)C)c1CC(=O)N=S(N)(=O)c1sc(C(C)(C)O)nc1CO] | (R) or (S)-N- (amino(4- (hydroxymethyl)-2- (2-hydroxypropan-2- yl)thiazol-5-yl)(oxo)- λ⁶-sulfaneylidene)-2- (4-(isochroman-6-yl)- 2,6- | CHIRAL- PAK IC, 2 * 25 cm, 5 um | EtOH in Hex (0.1% FA) | 586 |
| | | | diisopropylphenyl) | | | |
| | | | acetamide | | | |
| 272 | 222b | 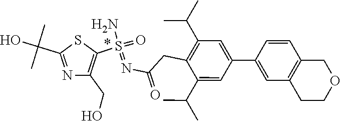 [SMILES: CC(C)c1cc(-c2ccc3c(c2)CCOC3)cc(C(C)C)c1CC(=O)N=S(N)(=O)c1sc(C(C)(C)O)nc1CO] | (S) or (R)-N- (amino(4- (hydroxymethyl)-2- (2-hydroxypropan-2- yl)thiazol-5-yl)(oxo)- λ⁶-sulfaneylidene)-2- (4-(isochroman-6-yl)- 2,6- | CHIRAL- PAK IC, 2 * 25 cm, 5 um | EtOH in Hex (0.1% FA) | 586 |
| | | | diisopropylphenyl) | | | |
| | | | acetamide | | | |
| 273 | 1330b | 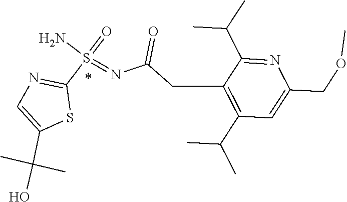 [SMILES: COCc1cc(C(C)C)c(CC(=O)N=S(N)(=O)c2ncc(C(C)(C)O)s2)c(C(C)C)n1] | (S) or (R)-N-(amino (5-(2- hydroxypropan-2-yl) thiazol-2-yl)(oxo)- λ⁶-sulfaneylidene)-2- (2,4-diisopropyl-6- (methoxymethyl) pyridin- 3-yl)acetamide | CHIRAL- PAK ID, 2 * 25 cm (5 um) | MeCN in CO₂ | 469 |
| 274 | 1330a | 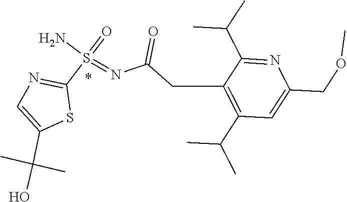 [SMILES: COCc1cc(C(C)C)c(CC(=O)N=S(N)(=O)c2ncc(C(C)(C)O)s2)c(C(C)C)n1] | (R) or (S)-N-(amino (5-(2- hydroxypropan-2-yl) thiazol-2-yl)(oxo)- λ⁶-sulfaneylidene)-2- (2,4-diisopropyl-6- (methoxymethyl) pyridin- 3-yl)acetamide | CHIRAL- PAK ID, 2 * 25 cm (5 um) | MeCN in CO₂ | 469 |
| 275 | 1324a | 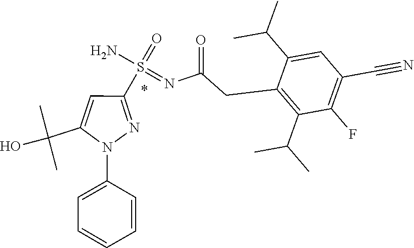 [SMILES: CC(C)c1cc(C#N)c(F)c(C(C)C)c1CC(=O)N=S(N)(=O)c1cc(C(C)(C)O)n(-c2ccccc2)n1] | (R) or (S)-N-(amino (5-(2- hydroxypropan-2-yl)- 1-phenyl-1H- pyrazol-3-yl)(oxo)-λ⁶- sulfaneylidene)- 2-(4-cyano-3- fluoro-2,6- diisopropylphenyl) acetamide | CHIRAL- PAK IC, 2 * 25 cm, 5 um | EtOH in Hex (0.1% FA) | 526 |
| 276 | 1324b | 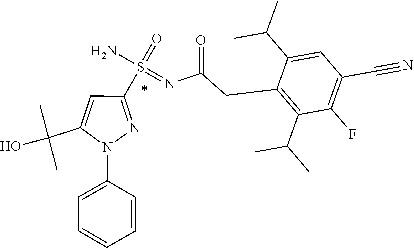 [SMILES: CC(C)c1cc(C#N)c(F)c(C(C)C)c1CC(=O)N=S(N)(=O)c1cc(C(C)(C)O)n(-c2ccccc2)n1] | (S) or (R)-N-(amino (5-(2- hydroxypropan-2-yl)- 1-phenyl-1H- pyrazol-3-yl)(oxo)-λ⁶- sulfaneylidene)- 2-(4-cyano-3- fluoro-2,6- diisopropylphenyl) acetamide | CHIRAL- PAK IC, 2 * 25 cm, 5 um | EtOH in Hex (0.1% FA) | 526 |
| 277 | 1319a | 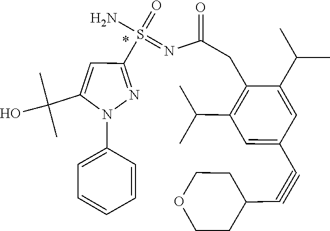 [SMILES: CC(C)c1cc(C#CC2CCOCC2)cc(C(C)C)c1CC(=O)N=S(N)(=O)c1cc(C(C)(C)O)n(-c2ccccc2)n1] | (R) or (S)-N-(amino (5-(2- hydroxypropan-2-yl)- 1-phenyl-1H- pyrazol-3-yl)(oxo-λ⁶- sulfaneylidene)- 2-(2,6-diisopropyl-4- ((tetrahydro-2H- pyran-4-yl)ethynyl) phenyl)acetamide | CHIRAL- PAK IG, 20 * 250 mm, 5 um | IPA in Hex (0.1% FA) | 591 |
| 278 | 1319b | 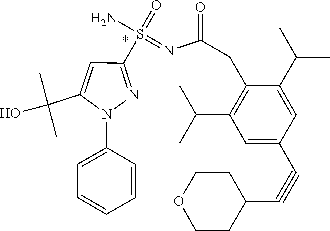 [SMILES: CC(C)c1cc(C#CC2CCOCC2)cc(C(C)C)c1CC(=O)N=S(N)(=O)c1cc(C(C)(C)O)n(-c2ccccc2)n1] | (S) or (R)-N- (amino(5-(2- hydroxypropan-2-yl)- 1-phenyl-1H- pyrazol-3-yl)(oxo)-λ⁶- sulfaneylidene)- 2-(2,6-diisopropyl-4- ((tetrahydro-2H- pyran-4-yl)ethynyl) phenyl)acetamide | CHIRAL- PAK IG, 20 * 250 mm, 5 um | IPA in Hex (0.1% FA) | 591 |
| 279 | 1308a | 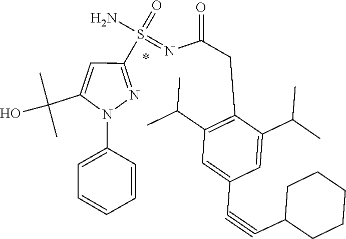 [SMILES: CC(C)c1cc(C#CC2CCCCC2)cc(C(C)C)c1CC(=O)N=S(N)(=O)c1cc(C(C)(C)O)n(-c2ccccc2)n1] | (R) or (S)-N- (amino(5-(2- hydroxypropan-2-yl)- 1-phenyl-1H- pyrazol-3-yl)(oxo)- λ⁶- sulfaneylidene)-2-(4- (cyclohexylethynyl)- 2,6- diisopropylphenyl) acetamide | Chiralpak ID, 2 * 25 cm, 5 um | IPA in Hex (0.1% FA) | 589 |
| 280 | 1308b | 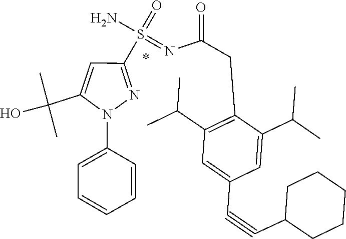 [SMILES: CC(C)c1cc(C#CC2CCCCC2)cc(C(C)C)c1CC(=O)N=S(N)(=O)c1cc(C(C)(C)O)n(-c2ccccc2)n1] | (S) or (R)-N-(amino (5-(2- hydroxypropan-2-yl)- 1-phenyl-1H- pyrazol-3-yl)(oxo)-λ⁶- sulfaneylidene)- 2-(4- (cyclohexylethynyl)- 2,6- diisopropylphenyl) acetamide | Chiralpak ID, 2 * 25 cm, 5 um | IPA in Hex (0.1% FA) | 589 |
| 281 | 1307a | 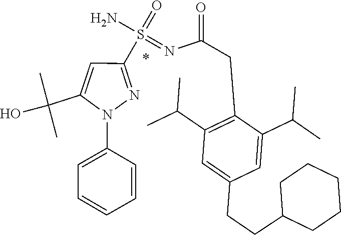 [SMILES: CC(C)c1cc(CCC2CCCCC2)cc(C(C)C)c1CC(=O)N=S(N)(=O)c1cc(C(C)(C)O)n(-c2ccccc2)n1] | (R) or (S)-N- (amino(5-(2- hydroxypropan-2-yl)- 1-phenyl-1H- pyrazol-3-yl)(oxo)-λ⁶- sulfaneylidene)- 2-(4-(2- cyclohexylethyl)- 2,6- diisopropylphenyl) acetamide | Chiralpak ID, 2 * 25 cm, 5 um | IPA in Hex (0.1% FA) | 593 |
| 282 | 1307b | 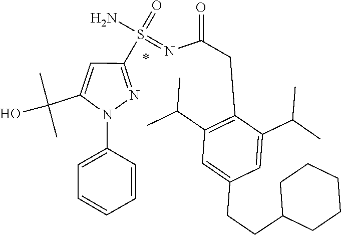 [SMILES: CC(C)c1cc(CCC2CCCCC2)cc(C(C)C)c1CC(=O)N=S(N)(=O)c1cc(C(C)(C)O)n(-c2ccccc2)n1] | (S) or (R)-N- (amino(5-(2- hydroxypropan-2- yl)-1-phenyl-1H- pyrazol-3-yl)(oxo)-λ⁶- sulfaneylidene)- 2-(4-(2- cyclohexylethyl)- 2,6- diisopropylphenyl) acetamide | Chiralpak ID, 2 * 25 cm, 5 um | IPA in Hex (0.1% FA) | 593 |
| 283 | 1313a | 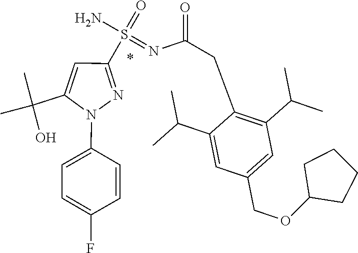 [SMILES: CC(C)c1cc(COC2CCCC2)cc(C(C)C)c1CC(=O)N=S(N)(=O)c1cc(C(C)(C)O)n(-c2ccc(F)cc2)n1] | (R) or (S)-N- (amino(1-(4- fluorophenyl)-5-(2- hydroxypropan-2- yl)-1H-pyrazol-3- yl)(oxo)-λ⁶- sulfaneylidene)-2-(4- ((cyclopentyloxy) methyl)-2,6- diisopropylphenyl) acetamide | CHIRAL- PAK IC, 2 * 25 cm, 5 um | 40% IPA in CO₂ | 599 |
| 284 | 1313b | 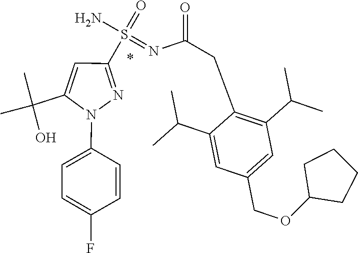 [SMILES: CC(C)c1cc(COC2CCCC2)cc(C(C)C)c1CC(=O)N=S(N)(=O)c1cc(C(C)(C)O)n(-c2ccc(F)cc2)n1] | (S) or (R)-N- (amino(1-(4- fluorophenyl)-5-(2- hydroxypropan-2- yl)-1H-pyrazol-3- yl)(oxo)-λ⁶- sulfaneylidene)-2-(4- ((cyclopentyloxy) methyl)-2,6- diisopropylphenyl) acetamide | CHIRAL- PAK IC, 2 * 25 cm, 5 um | 40% IPA in CO₂ | 599 |
| 285 | 1318a | 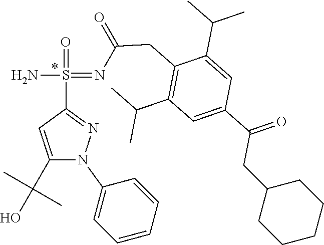 [SMILES: CC(C)c1cc(C(=O)CC2CCCCC2)cc(C(C)C)c1CC(=O)N=S(N)(=O)c1cc(C(C)(C)O)n(-c2ccccc2)n1] | (R) or (S)-N- (amino(5-(2- hydroxypropan-2-yl)- 1-phenyl-1H- pyrazol-3-yl)(oxo)-λ⁶- sulfaneylidene)- 2-(4-(2- cyclohexylacetyl)- 2,6- diisopropylphenyl) acetamide | CHIRAL- PAK IC, 2 * 25 cm, 5 um | EtOH in Hex (0.1% FA) | 607 |
| 286 | 1318b | 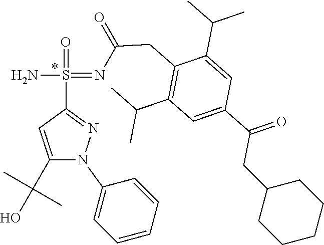 [SMILES: CC(C)c1cc(C(=O)CC2CCCCC2)cc(C(C)C)c1CC(=O)N=S(N)(=O)c1cc(C(C)(C)O)n(-c2ccccc2)n1] | (S) or (R)-N-(amino (5-(2- hydroxypropan-2-yl)- 1-phenyl-1H- pyrazol-3-yl)(oxo)-λ⁶- sulfaneylidene)- 2-(4-(2- cyclohexylacetyl)- 2,6- diisopropylphenyl) acetamide | CHIRAL- PAK IC, 2 * 25 cm, 5 um | EtOH in Hex (0.1% FA) | 607 |
| 287 | 305a | 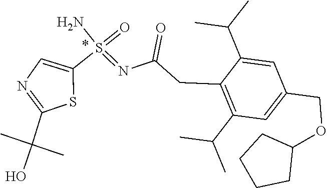 [SMILES: CC(C)c1cc(COC2CCCC2)cc(C(C)C)c1CC(=O)N=S(N)(=O)c1cnc(C(C)(C)O)s1] | (R) or (S)-N- (amino(2-(2- hydroxypropan-2-yl) thiazol-5-yl)(oxo)-λ⁶- sulfaneylidene)-2-(4- ((cyclopentyloxy) methyl)-2,6- diisopropylphenyl) acetamide | CHIRAL- PAK ID, 2 * 25 cm (5 um) | IPA in Hex (0.1% FA) | 522 |
| 288 | 305b | 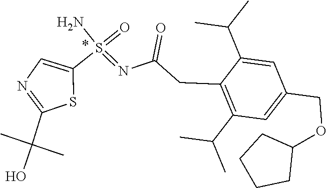 [SMILES: CC(C)c1cc(COC2CCCC2)cc(C(C)C)c1CC(=O)N=S(N)(=O)c1cnc(C(C)(C)O)s1] | (S) or (R)-N- (amino(2-(2- hydroxypropan-2-yl) thiazol-5-yl)(oxo)-λ⁶- sulfaneylidene)-2-(4- ((cyclopentyloxy) methyl)-2,6- diisopropylphenyl) acetamide | CHIRAL- PAK ID, 2 * 25 cm (5 um) | IPA in Hex (0.1% FA) | 522 |
| 289 | 1323a | 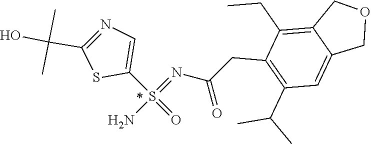 [SMILES: CCc1c2c(cc(C(C)C)c1CC(=O)N=S(N)(=O)c1cnc(C(C)(C)O)s1)COC2] | (R) or (S)-N- (amino(2-(2- hydroxypropan-2-yl) thiazol-5-yl)(oxo)- λ⁶-sulfaneylidene)- 2-(4-ethyl-6- isopropyl-1,3- dihydroisobenzofuran- 5-yl)acetamide | CHIRAL- PAK IC, 2 * 25 cm, 5 um | IPA in Hex (0.1% FA) | 452 |
| 290 | 1323b | 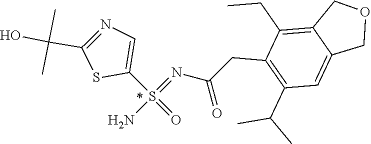 [SMILES: CCc1c2c(cc(C(C)C)c1CC(=O)N=S(N)(=O)c1cnc(C(C)(C)O)s1)COC2] | (S) or (R)-N- (amino(2-(2- hydroxypropan-2-yl) thiazol-5-yl)(oxo)- λ⁶-sulfaneylidene)- 2-(4-ethyl-6- isopropyl-1,3- dihydroisobenzofuran- 5-yl)acetamide | CHIRAL- PAK IC, 2 * 25 cm, 5 um | IPA in Hex (0.1% FA) | 452 |
| 291 | 1322a | 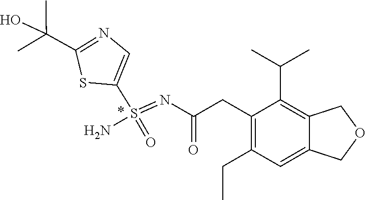 [SMILES: CCc1cc2c(c(C(C)C)c1CC(=O)N=S(N)(=O)c1cnc(C(C)(C)O)s1)COC2] | (R) or (S)-N- (amino(2-(2- hydroxypropan-2- yl)thiazol-5-yl)(oxo)- λ⁶-sulfaneylidene)- 2-(6-ethyl-4- isopropyl-1,3- dihydroisobenzofuran- 5-yl)acetamide | Chiralpak IA, 2 * 25 cm, 5 um | IPA in Hex (0.1% FA) | 452 |
| 292 | 1322b | 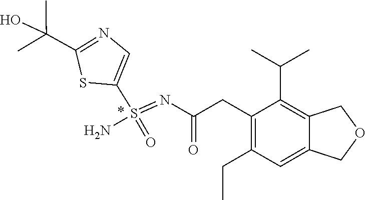 [SMILES: CCc1cc2c(c(C(C)C)c1CC(=O)N=S(N)(=O)c1cnc(C(C)(C)O)s1)COC2] | (S) or (R)-N- (amino(2-(2- hydroxypropan-2-yl) thiazol-5-yl)(oxo)- λ⁶-sulfaneylidene)- 2-(6-ethyl-4- isopropyl-1,3- dihydroisobenzofuran- 5-yl)acetamide | Chiralpak IA, 2 * 25 cm, 5 um | IPA in Hex (0.1% FA) | 452 |
| 293 | 1321a | 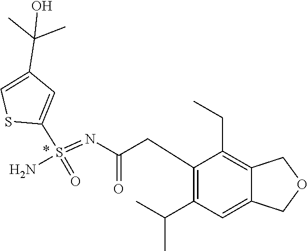 [SMILES: CCc1c2c(cc(C(C)C)c1CC(=O)N=S(N)(=O)c1cc(C(C)(C)O)cs1)COC2] | (R) or (S)-N- (amino(4-(2- hydroxypropan-2-yl) thiophen-2-yl)(oxo)- λ⁶-sulfaneylidene)- 2-(4-ethyl- 6-isopropyl-1,3- dihydroisobenzofuran- 5-yl)acetamide | CHIRAL- PAK IC, 2 * 25 cm, 5 um | EtOH in Hex (0.1% FA) | 451 |
| 294 | 1321b | 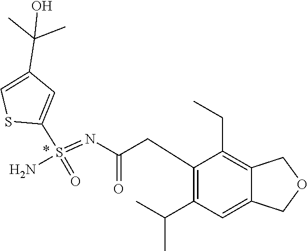 [SMILES: CCc1c2c(cc(C(C)C)c1CC(=O)N=S(N)(=O)c1cc(C(C)(C)O)cs1)COC2] | (S) or (R)-N- (amino(4-(2- hydroxypropan-2- yl)thiophen-2- yl)(oxo)-λ⁶- sulfaneylidene)-2-(4- ethyl-6-isopropyl-1,3- dihydroisobenzofuran- 5-yl)acetamide | CHIRAL- PAK IC, 2 * 25 cm, 5 um | EtOH in Hex (0.1% FA) | 451 |
| 295 | 1320a | 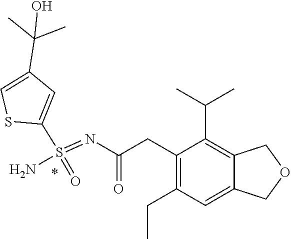 [SMILES: CCc1cc2c(c(C(C)C)c1CC(=O)N=S(N)(=O)c1cc(C(C)(C)O)cs1)COC2] | (R) or (S)-N- (amino(4-(2- hydroxypropan-2- yl)thiophen-2- yl)(oxo)-λ⁶- sulfaneylidene)-2-(6- ethyl-4-isopropyl-1,3- dihydroisobenzofuran- 5-yl)acetamide | CHIRAL- PAK IC, 2 * 25 cm, 5 um | EtOH in Hex (0.1% FA) | 451 |
| 296 | 1320b | 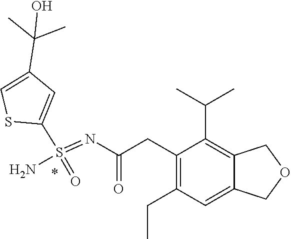 [SMILES: CCc1cc2c(c(C(C)C)c1CC(=O)N=S(N)(=O)c1cc(C(C)(C)O)cs1)COC2] | (S) or (R)-N- (amino(4-(2- hydroxypropan-2- yl)thiophen-2- yl)(oxo)-λ⁶- sulfaneylidene)-2-(6- ethyl-4-isopropyl-1,3- dihydroisobenzofuran- 5-yl)acetamide | CHIRAL- PAK IC, 2 * 25 cm, 5 um | EtOH in Hex (0.1% FA) | 451 |
| 297 | 1317a | 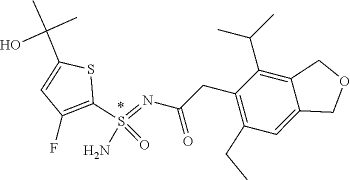 [SMILES: CCc1cc2c(c(C(C)C)c1CC(=O)N=S(N)(=O)c1sc(C(C)(C)O)cc1F)COC2] | (R) or (S)-N-(amino (3-fluoro-5-(2- hydroxypropan- 2-yl)thiophen-2- yl)(oxo)-λ⁶- sulfaneylidene)-2-(6- ethyl-4-isopropyl-1,3- dihydroisobenzofuran- 5-yl)acetamide | CHIRAL- PAK IC, 2 * 25 cm, 5 um | EtOH in Hex (0.1% FA) | 469 |
| 298 | 1317b | 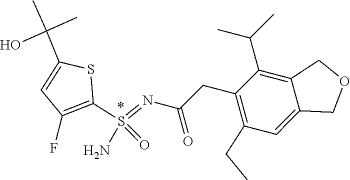 [SMILES: CCc1cc2c(c(C(C)C)c1CC(=O)N=S(N)(=O)c1sc(C(C)(C)O)cc1F)COC2] | (S) or (R)-N- (amino(3-fluoro-5-(2- hydroxypropan-2- yl)thiophen-2- yl)(oxo)-λ⁶- sulfaneylidene)-2-(6- ethyl-4-isopropyl-1,3- dihydroisobenzofuran- 5-yl)acetamide | CHIRAL- PAK IC, 2 * 25 cm, 5 um | EtOH in Hex (0.1% FA) | 469 |
| 299 | 1316b | 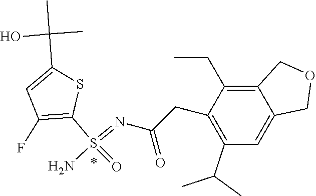 [SMILES: CCc1c2c(cc(C(C)C)c1CC(=O)N=S(N)(=O)c1sc(C(C)(C)O)cc1F)COC2] | (S) or (R)-N-(amino (3-fluoro-5-(2- hydroxypropan-2- yl)thiophen-2- yl)(oxo)-λ⁶- sulfaneylidene)-2-(4- ethyl-6-isopropyl-1,3- dihydroisobenzofuran- 5-yl)acetamide | CHIRAL- PAK IG, 20 * 250 mm, 5 um | EtOH in MTBE (10 mM NH₃—MeOH) | 469 |
| 300 | 1316a | 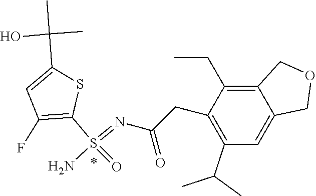 [SMILES: CCc1c2c(cc(C(C)C)c1CC(=O)N=S(N)(=O)c1sc(C(C)(C)O)cc1F)COC2] | (R) or (S)-N-(amino (3-fluoro-5-(2- hydroxypropan-2- yl)thiophen-2- yl)(oxo)-λ⁶- sulfaneylidene)-2-(4- ethyl-6-isopropyl-1,3- dihydroisobenzofuran- 5-yl)acetamide | CHIRAL- PAK IG, 20 * 250 mm, 5 um | EtOH in MTBE (10 mM NH₃—MeOH) | 469 |
| 301 | Ex. 301 1311a | 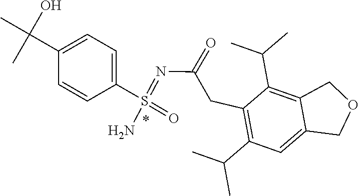 [SMILES: CC(C)c1cc2c(c(C(C)C)c1CC(=O)N=S(N)(=O)c1ccc(C(C)(C)O)cc1)COC2] | (R) or (S)-N- (amino(4-(2- hydroxypropan-2- yl)phenyl)(oxo)-λ⁶- sulfaneylidene)-2- (4,6-diisopropyl-1,3- dihydroisobenzofuran- 5-yl)acetamide | Chiralpak ID, 2 * 25 cm, 5 um | EtOH in Hex (0.1% FA) | 459 |
| 302 | 1311b | 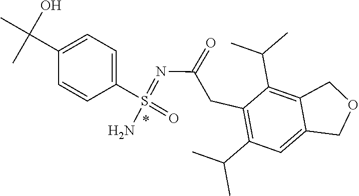 [SMILES: CC(C)c1cc2c(c(C(C)C)c1CC(=O)N=S(N)(=O)c1ccc(C(C)(C)O)cc1)COC2] | (S) or (R)-N- (amino(4-(2- hydroxypropan-2-yl) phenyl)(oxo)-λ⁶- sulfaneylidene)-2- (4,6-diisopropyl-1,3- dihydroisobenzofuran- 5-yl)acetamide | Chiralpak ID, 2 * 25 cm, 5 um | EtOH in Hex (0.1% FA) | 459 |
| 303 | 1306a | 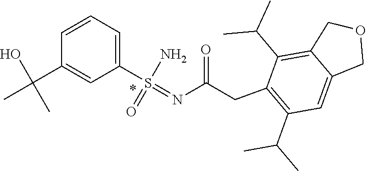 [SMILES: CC(C)c1cc2c(c(C(C)C)c1CC(=O)N=S(N)(=O)c1cccc(C(C)(C)O)c1)COC2] | (R) or (S)-N- (amino(3-(2- hydroxypropan-2-yl) phenyl)(oxo)-λ⁶- sulfaneylidene)-2- (4,6-diisopropyl-1,3- dihydroisobenzofuran- 5-yl)acetamide | Chiralpak ID, 2 * 25 cm, 5 um | EtOH in Hex (0.1% FA) | 459 |
| 304 | 1306b | 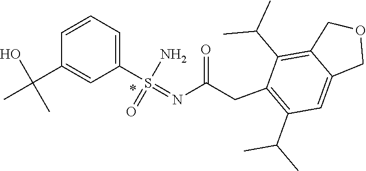 [SMILES: CC(C)c1cc2c(c(C(C)C)c1CC(=O)N=S(N)(=O)c1cccc(C(C)(C)O)c1)COC2] | (S) or (R)-N- (amino(3-(2- hydroxypropan-2-yl) phenyl)(oxo)-λ⁶- sulfaneylidene)-2- (4,6-diisopropyl-1,3- dihydroisobenzofuran- 5-yl)acetamide | Chiralpak ID, 2 * 25 cm, 5 um | EtOH in Hex (0.1% FA) | 459 |
| 305 | 1327b | 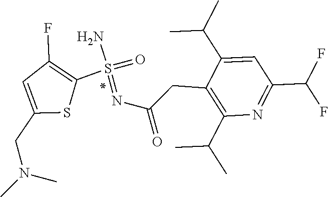 [SMILES: CC(C)c1cc(C(F)F)nc(C(C)C)c1CC(=O)N=S(N)(=O)c1sc(CN(C)C)cc1F] | (S) or (R)-N- (amino(5- ((dimethylamino) methyl)-3- fluorothiophen-2-yl) (oxo)-λ⁶- sulfaneylidene)-2- (6-(difluoromethyl)- 2,4- diisopropylpyridin- 3-yl)acetamide | CHIRAL- PAK IA, 2 * 25 cm, 5 um | EtOH in MTBE (10 mM NH₃—MeOH) | 491 |
| 306 | 1327a | 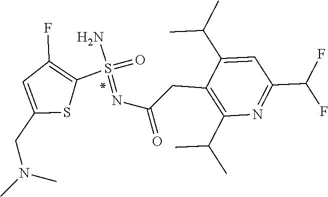 [SMILES: CC(C)c1cc(C(F)F)nc(C(C)C)c1CC(=O)N=S(N)(=O)c1sc(CN(C)C)cc1F] | (R) or (S)-N- (amino(5- ((dimethylamino) methyl)-3- fluorothiophen-2- yl)(oxo)-λ⁶- sulfaneylidene)-2- (6-(difluoromethyl)- 2,4- diisopropylpyridin- 3-yl)acetamide | CHIRAL- PAK IA, 2 * 25 cm, 5 um | EtOH in MTBE (10 mM NH₃—MeOH) | 491 |
| 307 | 1310a | 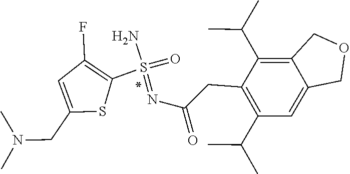 [SMILES: CC(C)c1cc2c(c(C(C)C)c1CC(=O)N=S(N)(=O)c1sc(CN(C)C)cc1F)COC2] | (R) or (S)-N- (amino(5- ((dimethylamino) methyl)-3- fluorothiophen-2- yl)(oxo)-λ⁶- sulfaneylidene)-2- (4,6-diisopropyl-1,3- dihydroisobenzofuran- 5-yl)acetamide | Chiralpak ID, 2 * 25 cm, 5 um | IPA in Hex:DCM = 3:1 (10 mM NH₃—MeOH) | 482 |
| 308 | 1310b | 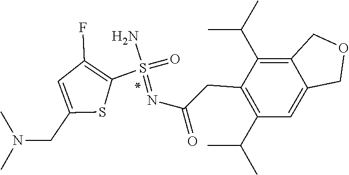 [SMILES: CC(C)c1cc2c(c(C(C)C)c1CC(=O)N=S(N)(=O)c1sc(CN(C)C)cc1F)COC2] | (S) or (R)-N- (amino(5- ((dimethylamino) methyl)-3- fluorothiophen-2- yl)(oxo)-λ⁶- sulfaneylidene)-2- (4,6-diisopropyl-1,3- dihydroisobenzofuran- 5-yl)acetamide | Chiralpak ID, 2 * 25 cm, 5 um | IPA in Hex:DCM = 3:1 (10 mM NH₃—MeOH) | 482 |
| 309 | 501b | 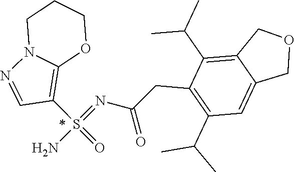 [SMILES: CC(C)c1cc2c(c(C(C)C)c1CC(=O)N=S(N)(=O)c1cnn3c1OCCC3)COC2] | (S) or (R)-N-(amino (6,7-dihydro-5H- pyrazolo[5,1-b][1,3] oxazin-3-yl)(oxo)- λ⁶-sulfaneylidene)-2- (4,6-diisopropyl-1,3- dihydroisobenzofuran- 5-yl)acetamide | CHIRAL ART Cellulose- SB, 2 * 25 cm, 5 um | EtOH in Hex (0.1% FA) | 447 |
| 310 | 501a | 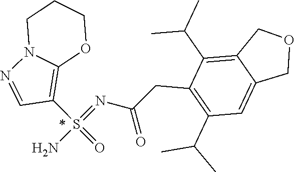 [SMILES: CC(C)c1cc2c(c(C(C)C)c1CC(=O)N=S(N)(=O)c1cnn3c1OCCC3)COC2] | (R) or (S)-N-(amino (6,7-dihydro-5H- pyrazolo[5,1-b][1,3] oxazin-3-yl)(oxo)- λ⁶-sulfaneylidene)-2- (4,6-diisopropyl-1,3- dihydroisobenzofuran- 5-yl)acetamide | CHIRAL ART Cellulose- SB, 2 * 25 cm, 5 um | EtOH in Hex (0.1% FA) | 447 |
| 311 | 504b | 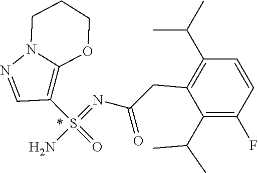 [SMILES: CC(C)c1ccc(F)c(C(C)C)c1CC(=O)N=S(N)(=O)c1cnn2c1OCCC2] | (S) or (R)-N-(amino (6,7-dihydro-5H- pyrazolo[5,1-b][1,3] oxazin-3-yl)(oxo)- λ⁶-sulfaneylidene)- 2-(3-fluoro-2,6- diisopropylphenyl) acetamide | CHIRAL ART Cellulose- SB, 2 * 25 cm, 5 um | EtOH in Hex (0.1% FA) | 423 |
| 312 | 504a | 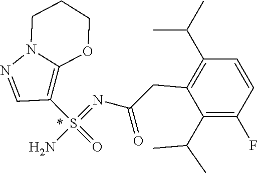 [SMILES: CC(C)c1ccc(F)c(C(C)C)c1CC(=O)N=S(N)(=O)c1cnn2c1OCCC2] | (R) or (S)-N-(amino (6,7-dihydro-5H- pyrazolo[5,1-b][1,3] oxazin-3-yl)(oxo)- λ⁶-sulfaneylidene)- 2-(3-fluoro-2,6- diisopropylphenyl) acetamide | CHIRAL ART Cellulose- SB, 2 * 25 cm, 5 um | EtOH in Hex (0.1% FA) | 423 |
| 313 | 1335a | 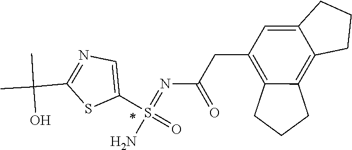 [SMILES: CC(C)(O)c1ncc(S(N)(=O)=NC(=O)Cc2cc3c(c4c2CCC4)CCC3)s1] | (R) or (S)-N- (amino(2-(2- hydroxypropan-2-yl) thiazol-5-yl)(oxo)- λ⁶-sulfaneylidene)- 2-(1,2,3,6,7,8- hexahydro-as- indacen- 4-yl)acetamide | CHIRAL- PAK IC, 2 * 25 cm, 5 um | EtOH in Hex (0.3% FA) | 420 |
| 314 | 1335b | 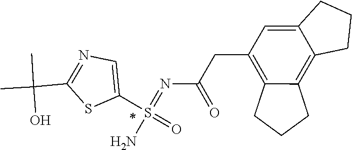 [SMILES: CC(C)(O)c1ncc(S(N)(=O)=NC(=O)Cc2cc3c(c4c2CCC4)CCC3)s1] | (S) or (R)-N- (amino(2-(2- hydroxypropan-2-yl) thiazol-5-yl)(oxo)- λ⁶-sulfaneylidene)- 2-(1,2,3,6,7,8- hexahydro-as- indacen- 4-yl)acetamide | CHIRAL- PAK IC, 2 * 25 cm, 5 um | EtOH in Hex (0.3% FA) | 420 |
| 315 | 205a | 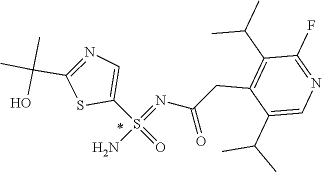 [SMILES: CC(C)c1cnc(F)c(C(C)C)c1CC(=O)N=S(N)(=O)c1cnc(C(C)(C)O)s1] | (R) or (S)-N- (amino(2-(2- hydroxypropan-2-yl) thiazol-5-yl)(oxo)- λ⁶-sulfaneylidene)- 2-(2-fluoro-3,5- diisopropylpyridin- 4-yl)acetamide | CHIRAL- PAK IG, 20 * 250 mm, 5 um | EtOH in Hex (0.1% FA) | 443 |
| 316 | 205b | 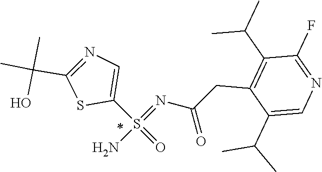 [SMILES: CC(C)c1cnc(F)c(C(C)C)c1CC(=O)N=S(N)(=O)c1cnc(C(C)(C)O)s1] | (S) or (R)-N- (amino(2-(2- hydroxypropan-2-yl) thiazol-5-yl)(oxo)- λ⁶-sulfaneylidene)- 2-(2-fluoro-3,5- diisopropylpyridin- 4-yl)acetamide | CHIRAL- PAK IG, 20 * 250 mm, 5 um | EtOH in Hex (0.1% FA) | 443 |
| 317 | 1331a | 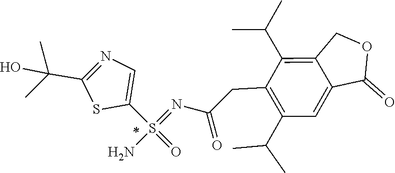 [SMILES: CC(C)c1cc2c(c(C(C)C)c1CC(=O)N=S(N)(=O)c1cnc(C(C)(C)O)s1)COC2=O] | (R) or (S)-N- (amino(2-(2- hydroxypropan-2-yl) thiazol-5-yl)(oxo)- λ⁶-sulfaneylidene)-2- (4,6-diisopropyl- 1-oxo-1,3- dihydroisobenzofuran- 5-yl)acetamide | CHIRAL- PAK IC, 2 * 25 cm, 5 um | EtOH in MTBE (10 mM NH₃—MeOH) | 480 |
| 318 | 1331b | 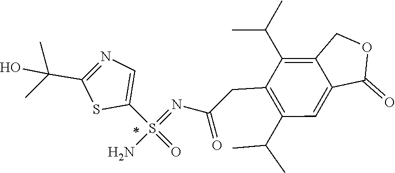 [SMILES: CC(C)c1cc2c(c(C(C)C)c1CC(=O)N=S(N)(=O)c1cnc(C(C)(C)O)s1)COC2=O] | (S) or (R)-N- (amino(2-(2- hydroxypropan-2-yl) thiazol-5-yl)(oxo)- λ⁶-sulfaneylidene)-2- (4,6-diisopropyl- 1-oxo-1,3- dihydroisobenzofuran- 5-yl)acetamide | CHIRAL- PAK IC, 2 * 25 cm, 5 um | EtOH in MTBE (10 mM NH₃—MeOH) | 480 |
| 319 | 1328b | 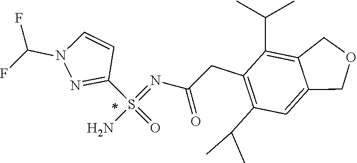 [SMILES: CC(C)c1cc2c(c(C(C)C)c1CC(=O)N=S(N)(=O)c1ccn(C(F)F)n1)COC2] | (R) or (S)-N- (amino(1- (difluoromethyl)- 1H-pyrazol-3- yl)(oxo)-λ⁶- sulfaneylidene)-2- (4,6-diisopropyl-1,3- dihydroisobenzofuran- 5-yl)acetamide | Chiralpak ID, 2 * 25 cm, 5 um | IPA in Hex (0.1% FA) | 441 |
| 320 | 1328a | 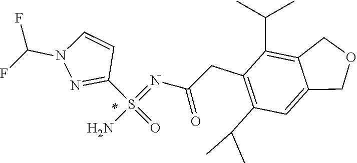 [SMILES: CC(C)c1cc2c(c(C(C)C)c1CC(=O)N=S(N)(=O)c1ccn(C(F)F)n1)COC2] | (S) or (R)-N- (amino(1- (difluoromethyl)- 1H-pyrazol-3- yl)(oxo)-λ⁶- sulfaneylidene)-2- (4,6-diisopropyl-1,3- dihydroisobenzofuran- 5-yl)acetamide | Chiralpak ID, 2 * 25 cm, 5 um | IPA in Hex (0.1% FA) | 441 |
| 321 | 265a | 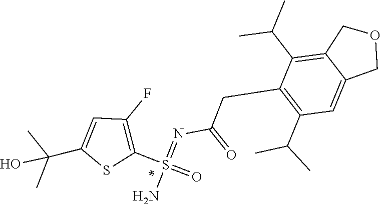 [SMILES: CC(C)c1cc2c(c(C(C)C)c1CC(=O)N=S(N)(=O)c1sc(C(C)(C)O)cc1F)COC2] | (R) or (S)-N-(amino (3-fluoro-5-(2- hydroxypropan-2- yl)thiophen-2- yl)(oxo)-λ⁶- sulfaneylidene)-2- (4,6- diisopropyl-1,3- dihydroisobenzofuran- 5-yl)acetamide | CHIRAL- PAK IG, 2 * 25 cm (5 um) | EtOH in Hex (8 mm NH₃ • MeOH) | 483 |
| 322 | 265b | 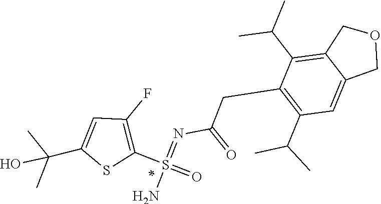 [SMILES: CC(C)c1cc2c(c(C(C)C)c1CC(=O)N=S(N)(=O)c1sc(C(C)(C)O)cc1F)COC2] | (S) or (R)-N-(amino (3-fluoro-5-(2- hydroxypropan-2- yl)thiophen-2- yl)(oxo)-λ⁶- sulfaneylidene)-2- (4,6- diisopropyl-1,3- dihydroisobenzofuran- 5-yl)acetamide | CHIRAL- PAK IG, 2 * 25 cm (5 um) | EtOH in Hex (8 mM NH₃ • MeOH) | 483 |
| 323 | 1326a | 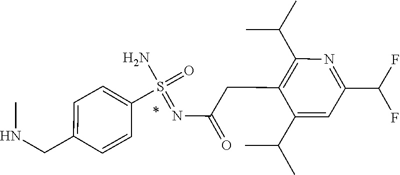 [SMILES: CNCc1ccc(S(N)(=O)=NC(=O)Cc2c(C(C)C)cc(C(F)F)nc2C(C)C)cc1] | (S) or (R)-N- (amino(4- ((methylamino) methyl)phenyl)(oxo)- λ⁶-sulfaneylidene)-2- (6- (difluoromethyl)-2,4- diisopropylpyridin-3- yl)acetamide | chiralpak AS, 2 * 25 cm (5 um) | 20% IPA (2 mm NH₃—MeOH) in CO₂ | 453 |
| 324 | 1326b | 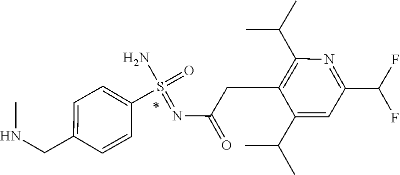 [SMILES: CNCc1ccc(S(N)(=O)=NC(=O)Cc2c(C(C)C)cc(C(F)F)nc2C(C)C)cc1] | (R) or (S)-N-(amino (4-((methylamino) methyl)phenyl)(oxo)- λ⁶-sulfaneylidene)-2- (6- (difluoromethyl)-2,4- diisopropylpyridin-3- yl)acetamide | chiralpak AS, 2 * 25 cm (5 um) | 20% IPA (2 mM NH₃—MeOH) in CO₂ | 453 |
| 325 | 1338a | 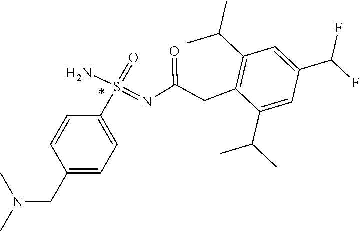 [SMILES: CC(C)c1cc(C(F)F)cc(C(C)C)c1CC(=O)N=S(N)(=O)c1ccc(CN(C)C)cc1] | (R) or (S)-N-(amino (4-((dimethylamino) methyl)phenyl)(oxo)- λ₆-sulfaneylidene)- 2-(4- (difluoromethyl)-2,6- diisopropylphenyl) acetamide | CHIRAL- PAK IG, 20 * 250 mm, 5 um | IPA in Hex | 466 |
| 326 | 1338b | 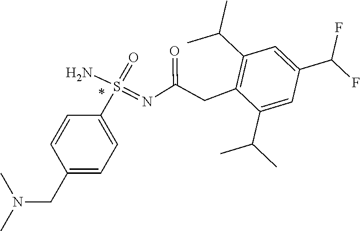 [SMILES: CC(C)c1cc(C(F)F)cc(C(C)C)c1CC(=O)N=S(N)(=O)c1ccc(CN(C)C)cc1] | (S) or (R)-N- (amino(4- ((dimethylamino) methyl)phenyl)(oxo)- λ⁶-sulfaneylidene)-2- (4- (difluoromethyl)-2,6- diisopropylphenyl) acetamide | CHIRAL- PAK IG, 20 * 250 mm, 5 um | IPA in Hex | 466 |
| 327 | 1332a | 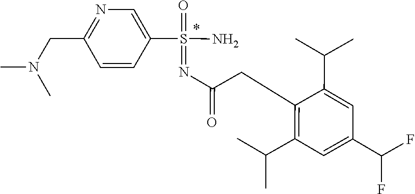 [SMILES: CC(C)c1cc(C(F)F)cc(C(C)C)c1CC(=O)N=S(N)(=O)c1ccc(CN(C)C)nc1] | (S) or (R)-N- (amino(6- ((dimethylamino) methyl)pyridin-3- yl)(oxo)-λ⁶- sulfaneylidene)-2-(4- (difluoromethyl)-2,6- diisopropylphenyl) acetamide | CHIRAL- PAK AD, 2 * 25 cm, 5 um | 20% EtOH (2 mM NH₃—MeOH) in CO₂ | 467 |
| 328 | 1332b | 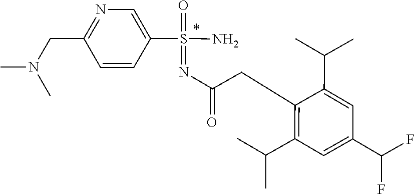 [SMILES: CC(C)c1cc(C(F)F)cc(C(C)C)c1CC(=O)N=S(N)(=O)c1ccc(CN(C)C)nc1] | (R) or (S)-N- (amino(6- ((dimethylamino) methyl)pyridin-3- yl)(oxo)-λ⁶- sulfaneylidene)-2-(4- (difluoromethyl)-2,6- diisopropylphenyl) acetamide | CHIRAL- PAK AD, 2 * 25 cm, 5 um | 20% EtOH (2 mM NH₃—MeOH) in CO₂ | 467 |
| 329 | 1325b | 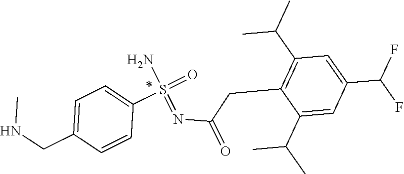 [SMILES: CNCc1ccc(S(N)(=O)=NC(=O)Cc2c(C(C)C)cc(C(F)F)cc2C(C)C)cc1] | (S) or (R)-N- (amino(4- ((methylamino) methyl)phenyl)(oxo)- λ⁶-sulfaneylidene)-2- (4- (difluoromethyl)-2,6- diisopropylphenyl) acetamide | CHIRAL- PAK AS, 2 * 25 cm (5 um) | 20% IPA (2 mM NH₃—MeOH) in CO₂ | 452 |
| 330 | 1325a | 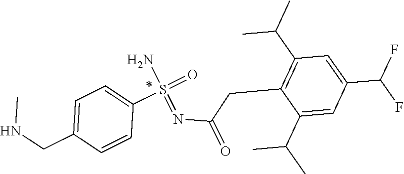 [SMILES: CNCc1ccc(S(N)(=O)=NC(=O)Cc2c(C(C)C)cc(C(F)F)cc2C(C)C)cc1] | (R) or (S)-N- (amino(4- ((methylamino) methyl)phenyl)(oxo)- λ⁶-sulfaneylidene)-2- (4- (difluoromethyl)-2,6- diisopropylphenyl) acetamide | CHIRAL- PAK AS, 2 * 25 cm (5 um) | 20% IPA (2 mM NH₃—MeOH) in CO₂ | 452 |
| 331 | 1329a | 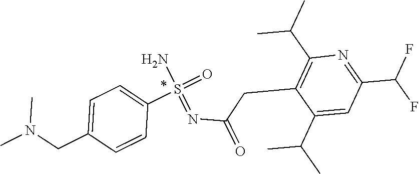 [SMILES: CC(C)c1cc(C(F)F)nc(C(C)C)c1CC(=O)N=S(N)(=O)c1ccc(CN(C)C)cc1] | (R) or (S)-N- (amino(4- ((dimethylamino) methyl)phenyl)(oxo)- λ⁶-sulfaneylidene)-2- (6- (difluoromethyl)-2,4- diisopropylpyridin-3- yl)acetamide | CHIRAL- PAK IC, 2 * 25 cm, 5 um | EtOH in Hex (8 mM NH₃ • MeOH) | 467 |
| 332 | 1329b | 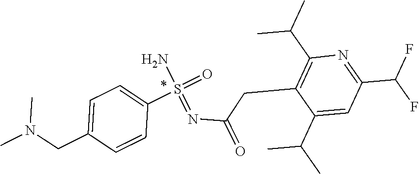 [SMILES: CC(C)c1cc(C(F)F)nc(C(C)C)c1CC(=O)N=S(N)(=O)c1ccc(CN(C)C)cc1] | (S) or (R)-N- (amino(4- ((dimethylamino) methyl)phenyl)(oxo)- λ⁶-sulfaneylidene)-2- (6-(difluoromethyl)- 2,4- diisopropylpyridin- 3-yl)acetamide | CHIRAL- PAK IC, 2 * 25 cm, 5 um | EtOH in Hex (8 mM NH₃ • MeOH) | 467 |
| 333 | 1315f | 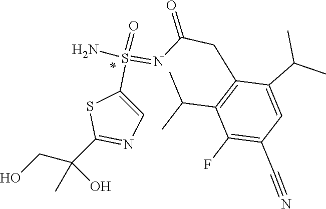 [SMILES: CC(C)c1cc(C#N)c(F)c(C(C)C)c1CC(=O)N=S(N)(=O)c1cnc(C(C)(O)CO)s1] | (R, R) and (S, R)- N-(amino(2-(1,2- dihydroxypropan- 2-yl)thiazol-5- yl)(oxo)-λ⁶- sulfaneylidene)-2- (4-cyano- 3-fluoro-2,6- diisopropylphenyl) acetamide (Two isomers) | 1^{st} and 2^{nd} peak CHIRAL- PAK IC, 2 * 25 cm, 5 um | IPA in Hex (0.1% FA) | 483 |
| 334 | 1315e | 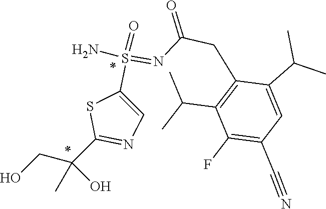 [SMILES: CC(C)c1cc(C#N)c(F)c(C(C)C)c1CC(=O)N=S(N)(=O)c1cnc(C(C)(O)CO)s1] | (S, S) or (R, S)-N- (amino(2-(1,2- dihydroxypropan- 2-yl)thiazol-5- yl)(oxo)-λ⁶- sulfaneylidene)-2- (4-cyano- 3-fluoro-2,6- diisopropylphenyl) acetamide | 3^{rd} peak CHIRAL- PAK IC, 2 * 25 cm, 5 um | IPA in Hex (0.1% FA) | 483 |
| 335 | 1315d | 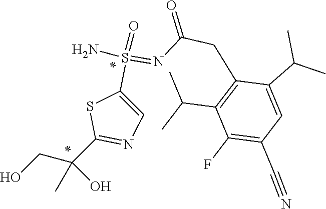 [SMILES: CC(C)c1cc(C#N)c(F)c(C(C)C)c1CC(=O)N=S(N)(=O)c1cnc(C(C)(O)CO)s1] | (R, S) or (S, S)- N-(amino(2-(1,2- dihydroxypropan- 2-yl)thiazol-5- yl)(oxo)-λ⁶- sulfaneylidene)-2-(4- cyano-3-fluoro-2,6- diisopropylphenyl) acetamide | 4^{th} peak CHIRAL- PAK IC, 2 * 25 cm, 5 um | IPA in Hex (0.1% FA) | 483 |
| 336 | 1315b | 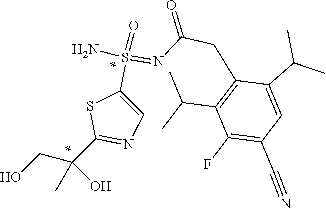 [SMILES: CC(C)c1cc(C#N)c(F)c(C(C)C)c1CC(=O)N=S(N)(=O)c1cnc(C(C)(O)CO)s1] | (R, R) or (S, R)- N-(amino(2-(1,2- dihydroxypropan-2- yl)thiazol-5- yl)(oxo)-λ⁶- sulfaneylidene)-2-(4- cyano-3-fluoro-2,6- diisopropylphenyl) acetamide | CHIRAL- PAK IC, 2 * 25 cm, 5 um; from Ex. 333 | IPA in Hex (0.1% FA) | 483 |
| 337 | 1315a | 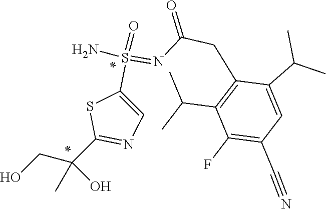 [SMILES: CC(C)c1cc(C#N)c(F)c(C(C)C)c1CC(=O)N=S(N)(=O)c1cnc(C(C)(O)CO)s1] | (S, R) or (R, R)-N- (amino(2-(1,2- dihydroxypropan- 2-yl)thiazol-5- yl)(oxo)-λ⁶- sulfaneylidene)- 2-(4-cyano- 3-fluoro-2,6- diisopropylphenyl) acetamide | CHIRAL- PAK IC, 2 * 25 cm, 5 um; from Ex. 333 | IPA in Hex (0.1% FA) | 483 |
| 338 | 1312b | 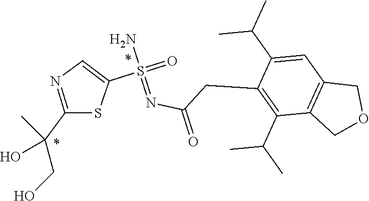 [SMILES: CC(C)c1cc2c(c(C(C)C)c1CC(=O)N=S(N)(=O)c1cnc(C(C)(O)CO)s1)COC2] | (R, R) and (S, R)- N-(amino(2-(1,2- dihydroxypropan- 2-yl)thiazol-5- yl)(oxo)-λ⁶- sulfaneylidene)-2- (4,6-diisopropyl-1,3- dihydroisobenzofuran- 5-yl)acetamide (Two isomers) | 1^{st} and 2^{nd} peak CHIRAL- PAK AD, 2 * 25 cm, 5 um; from Ex. 246 | EtOH in Hex (0.1% FA) | 482 |
| 339 | 1312a | 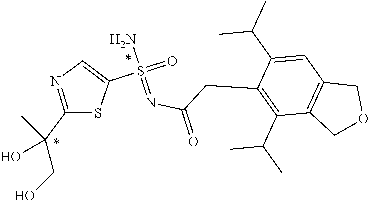 [SMILES: CC(C)c1cc2c(c(C(C)C)c1CC(=O)N=S(N)(=O)c1cnc(C(C)(O)CO)s1)COC2] | (R, S) or (S, S)-N- (amino(2-(1,2- dihydroxypropan-2- yl)thiazol-5- yl)(oxo)-λ⁶- sulfaneylidene)-2- (4,6-diisopropyl-1,3- dihydroisobenzofuran- 5-yl)acetamide | 4^{th} peak CHIRAL- PAK AD, 2 * 25 cm, 5 um; from Ex. 252 | EtOH in Hex (0.1% FA) | 482 |

TABLE 38
Examples in the following table were prepared using similar conditions as described in
Example 241 and Scheme III from appropriate starting materials.

| | | | | Exact |
|---|---|---|---|---|
| Ex. | Final | | | Mass |
| # | Target # | Structure | IUPAC Name | [M + H]⁺ |
| 340 | 1343a | 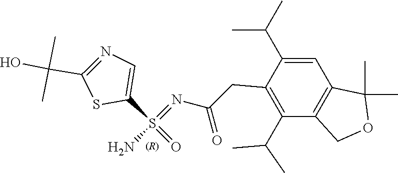 | (R)-N-(amino(2-(2-hydroxypropan-2-yl)thiazol-5- yl)(oxo)-λ⁶-sulfaneylidene)-2-(4,6-diisopropyl-1,1- dimethyl-1,3-dihydroisobenzofuran-5-yl)acetamide | 494 |

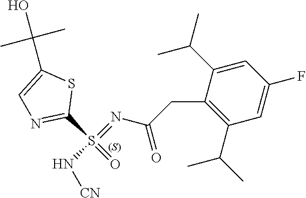

### (S)—N-(cyanamido(5-(2-hydroxypropan-2-yl)thiazol-2-yl)(oxo)-λ⁶-sulfaneylidene)-2-(4-fluoro-2,6-diisopropylphenyl)acetamide (Scheme V)

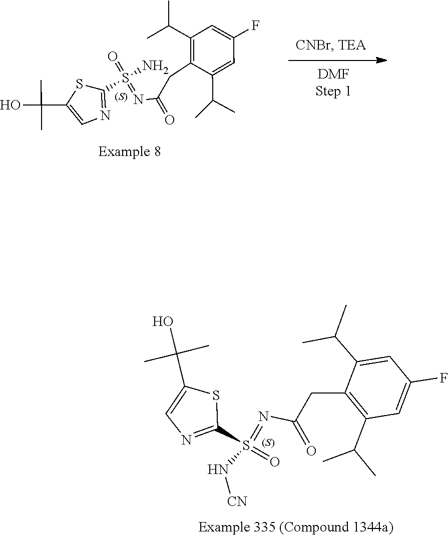

To a stirred solution of (S)—N-(amino(5-(2-hydroxypropan-2-yl)thiazol-2-yl)(oxo)-λ⁶-sulfaneylidene)-2-(4-fluoro-2,6-diisopropylphenyl)acetamide (200 mg, 0.45 mmol) in DMF (10 mL) in a 50 mL round-bottom flask was added TEA (184 mg, 1.8 mmol) at RT, followed by the addition of cyanic bromide (96 mg, 0.91 mmol) in portions at RT. The resulting solution was stirred for 4 h at RT. The pH value of the solution was adjusted to 10 with NaOH (1 M). The resulting mixture was concentrated under vacuum. The crude product was purified by Flash-Prep-HPLC with the following conditions: XSelect CSH Prep C18 OBD Column, 5 um, 19*150 mm; Mobile Phase A: water (10 mM NH₄HCO_{3+0.1}% NH₃.H₂O), Mobile Phase B: ACN; Flow rate: 25 mL/min; Gradient: 26% B to 44% B over 7 min; 254/210 nm; Rt₁: 6.35 min. This resulted in 15.2 mg (7.2%) of Example 335 as a white solid. MS-ESI: 467 (M+1). ¹H NMR (300 MHz, DMSO-d₆) δ 7.66 (s, 1H), 6.99 (br s, 1H), 6.83 (d, J=10.5 Hz, 2H), 5.80 (s, 1H), 3.58 (s, 2H), 3.18-3.09 (m, 2H), 1.52 (s, 6H), 1.13-1.07 (m, 12H). ¹⁹F NMR (300 MHz, DMSO-d₆) δ −116.

TABLE 39
Examples in the following table were prepared using similar conditions as described in
Example 335 and Scheme V from appropriate starting material.

| | | | | Exact |
|---|---|---|---|---|
| | Final | | | Mass |
| Ex. # | Target # | Structure | IUPAC Name | [M + H]⁺ |
| 342 | 1344b | 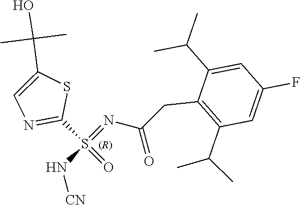 | (R)-N-(cyanamido(5-(2-hydroxypropan-2-yl)thiazol- 2-yl)(oxo)-λ⁶-sulfaneylidene)-2-(4-fluoro-2,6- diisopropylphenyl)acetamide (prepared from Ex. 9) | 467 |
| 343 | 1345a | 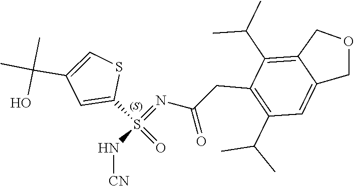 | (S)-N-(cyanamido(4-(2-hydroxypropan-2- yl)thiophen-2-yl)(oxo)-λ⁶-sulfaneylidene)-2-(4,6- diisopropyl-1,3-dihydroisobenzofuran-5- yl)acetamide (prepared from Ex. 222) | 490 |
| 344 | 1345b | 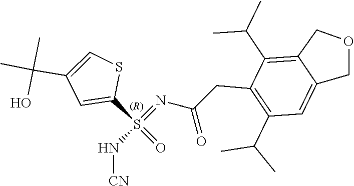 | (R)-N-(cyanamido(4-(2-hydroxypropan-2- yl)thiophen-2-yl)(oxo)-λ⁶-sulfaneylidene)-2-(4,6- diisopropyl-1,3-dihydroisobenzofuran-5- yl)acetamide (prepared from Ex. 223) | 490 |
| 345 | 1346a | 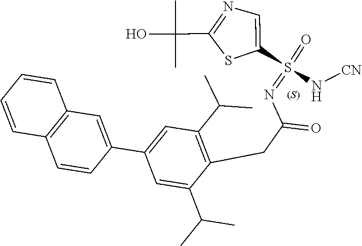 | (S)-N-(cyanamido(2-(2-hydroxypropan-2-yl)thiazol- 5-yl)(oxo)-λ⁶-sulfaneylidene)-2-(2,6-diisopropyl-4- (naphthalen-2-yl)phenyl)acetamide (prepared from Ex. 169) | 575 |
| 346 | 1346b | 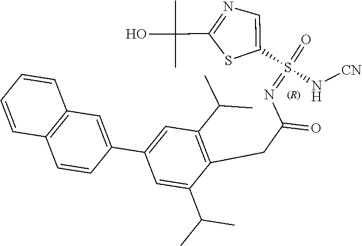 | (R)-N-(cyanamido(2-(2-hydroxypropan-2-yl)thiazol- 5-yl)(oxo)-λ⁶-sulfaneylidene)-2-(2,6-diisopropyl-4- (naphthalen-2-yl)phenyl)acetamide (prepared from Ex. 170) | 575 |

### Example 347

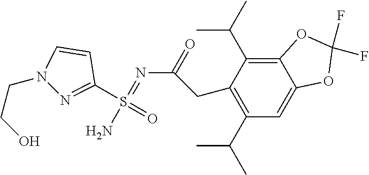 [SMILES: CC(C)c1cc2c(c(C(C)C)c1CC(=O)N=S(N)(=O)c1ccn(CCO)n1)OC(F)(F)O2]

### N-(amino(1-(2-hydroxyethyl)-1H-pyrazol-3-yl)(oxo)-λ⁶-sulfaneylidene)-2-(2,2-difluoro-4,6-diisopropylbenzo[d][1,3]dioxol-5-yl)acetamide (Scheme VI)

### Examples 348 and 349

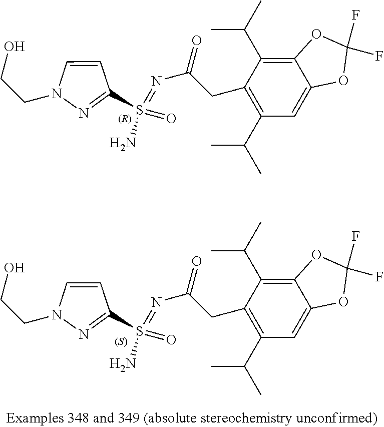

### (R) and (S)—N-(amino(1-(2-hydroxyethyl)-1H-pyrazol-3-yl)(oxo)-λ⁶-sulfaneylidene)-2-(2,2-difluoro-4,6-diisopropylbenzo[d][1,3]dioxol-5-yl)acetamide

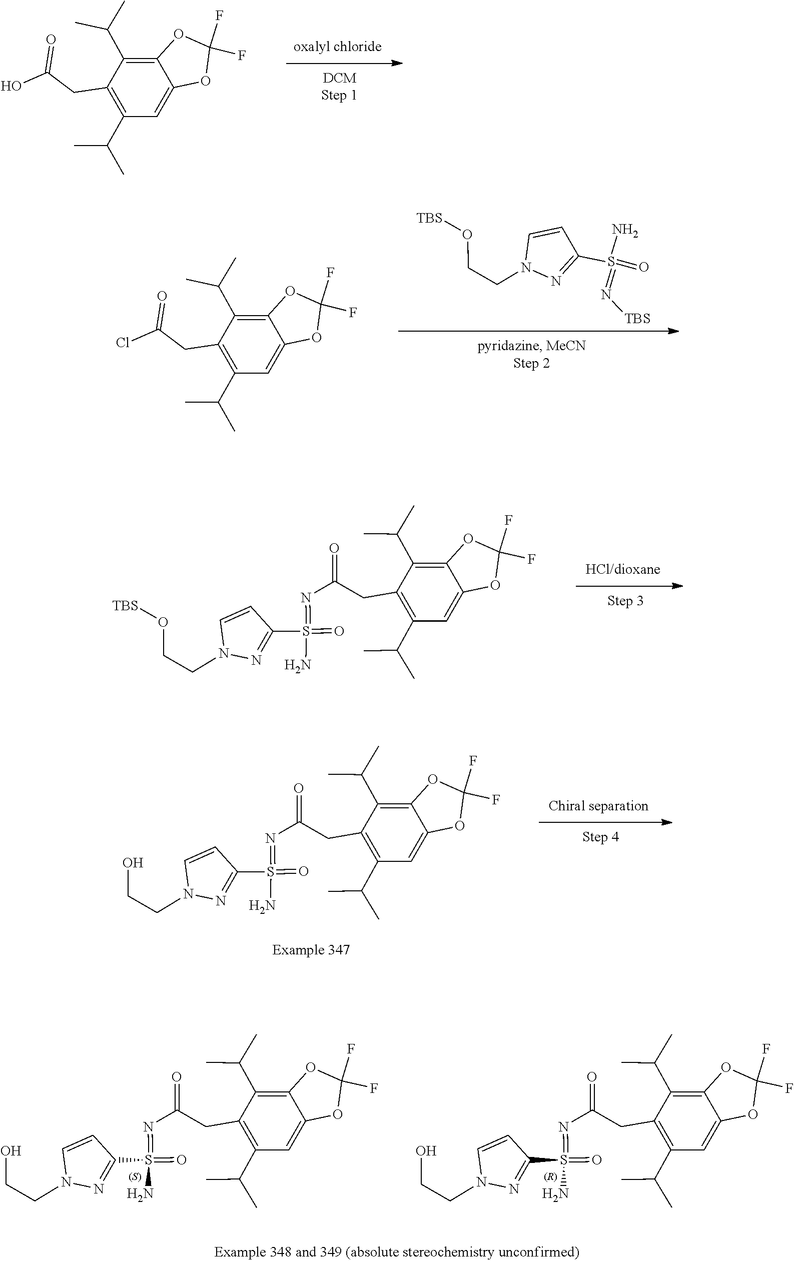

### Step 1: 2-(2,2-Difluoro-4,6-diisopropylbenzo[d][1,3]dioxol-5-yl)acetyl Chloride

To a stirred solution of (2,2-difluoro-4,6-diisopropyl-1,3-benzodioxol-5-yl)acetic acid (200 mg, 0.67 mmol) in DCM (6.0 mL) under nitrogen were added oxalyl chloride (845 mg, 6.66 mmol) and DMF (cat.) dropwise at 0° C. The reaction mixture was stirred for 2 h at RT. The reaction mixture was concentrated under vacuum. This resulted in 200 mg (crude) of the title product as brown oil.

### Step 2: N-(amino(1-(2-((tert-butyldimethylsilyl)oxy)ethyl)-1H-pyrazol-3-yl)(oxo)-λ⁶-sulfaneylidene)-2-(2,2-difluoro-4,6-diisopropylbenzo[d][1,3]dioxol-5-yl)acetamide

To a stirred solution of 2-(2,2-difluoro-4,6-diisopropylbenzo[d][1,3]dioxol-5-yl)acetyl chloride (200 mg, crude from the last step) in ACN (6.0 mL) under nitrogen was added pyridazine (60 mg, 0.75 mmol) at RT. The reaction mixture was stirred for 10 min at RT. Then to the above mixture was added N-(tert-butyldimethylsilyl)-1-(2-((tert-butyldimethylsilyl)oxy)ethyl)-1H-pyrazole-3-sulfonimidamide (263 mg, 0.63 mmol) in ACN (2.0 mL) dropwise at 0° C. The reaction solution was stirred overnight at RT. The reaction solution was concentrated under vacuum. The residue was purified by prep-TLC (PE/EtOAc 2:1). This resulted in 260 mg (66%, over two steps) of the title compound as yellow oil. MS-ESI: 587 (M+1).

### Step 3: N-(amino(1-(2-hydroxyethyl)-1H-pyrazol-3-yl)(oxo)-λ⁶-sulfaneylidene)-2-(2,2-difluoro-4,6-diisopropylbenzo[d][1,3]dioxol-5-yl)acetamide

A solution of N-(amino(1-(2-((tert-butyldimethylsilyl)oxy)ethyl)-1H-pyrazol-3-yl)(oxo)-λ⁶-sulfaneylidene)-2-(2,2-difluoro-4,6-diisopropylbenzo[d][1,3]dioxol-5-yl)acetamide (260 mg, 0.44 mmol) in HCl/dioxane (4.0 M, 2.0 mL, 8.0 mmol) was stirred for 1 h at RT. The pH value of the solution was adjusted to 8 with sat. Na₂CO₃ (aq.). The mixture was concentrated under vacuum. The crude product was purified by prep-HPLC using the following conditions: XSelect CSH Prep C18 OBD Column, 5 um, 19*150 mm; Mobile Phase A: water (10 mM NH₄HCO₃+0.1% NH₃.H₂O), Mobile Phase B: ACN; Flow rate: 25 mL/min; Gradient: 22% B to 40% B over 7 min; 210/254 nm; Rt: 5.78 min. This resulted in 110 mg (52%) of Example 347 as a white solid. MS-ESI: 473 (M+1). ¹H NMR (400 MHz, DMSO-d₆) δ 7.83 (d, J=2.4 Hz, 1H), 7.61 (br s, 2H), 7.13 (s, 1H), 6.60 (d, J=2.4 Hz, 1H), 4.98 (t, J=5.2 Hz, 1H), 4.19 (t, J=5.4 Hz, 2H), 3.76-3.72 (m, 2H), 3.67 (s, 2H), 3.18-3.08 (m, 2H), 1.21 (d, J=6.9 Hz, 3H), 1.17 (d, J=6.9 Hz, 3H), 1.11 (d, J=6.7 Hz, 3H), 1.08 (d, J=6.7 Hz, 3H).

### Step 4: Chiral Separation

Example 347 (100 mg, 0.21 mmol) was resolved by prep-chiral HPLC using the following conditions: CHIRALPAK ID, 2*25 cm (5 um); Mobile Phase A: Hex (0.1% FA), Mobile Phase B: IPA; Flow rate: 16 mL/min; Gradient: 18% B to 18% B over 30 min; UV 254/220 nm; Rt₁: 8.397 min (Example 348); Rt₂: 16.153 min (Example 349). This resulted in 39.4 mg of Example 348 followed by 51.4 mg of Example 349, both as off-white solid.

Example 348: MS-ESI: 473 (M+1). ¹H NMR (400 MHz, DMSO-d₆) δ 7.84 (d, J=2.4 Hz, 1H), 7.69 (br s, 2H), 7.13 (s, 1H), 6.61 (d, J=2.4 Hz, 1H), 4.98 (t, J=5.2 Hz, 1H), 4.19 (t, J=5.4 Hz, 2H), 3.76-3.72 (m, 2H), 3.63 (s, 2H), 3.17-3.08 (m, 2H), 1.21 (d, J=6.9 Hz, 3H), 1.17 (d, J=6.9 Hz, 3H), 1.11 (d, J=6.7 Hz, 3H), 1.08 (d, J=6.7 Hz, 3H).

Example 349: MS-ESI: 473 (M+1). ¹H NMR (400 MHz, DMSO-d₆) δ 7.84 (d, J=2.4 Hz, 1H), 7.67 (br s, 2H), 7.13 (s, 1H), 6.61 (d, J=2.4 Hz, 1H), 4.98 (t, J=5.2 Hz, 1H), 4.19 (t, J=5.4 Hz, 2H), 3.76-3.72 (m, 2H), 3.67 (s, 2H), 3.16-3.09 (m, 2H), 1.21 (d, J=6.9 Hz, 3H), 1.17 (d, J=6.9 Hz, 3H), 1.11 (d, J=6.7 Hz, 3H), 1.08 (d, J=6.7 Hz, 3H).

### Example 350

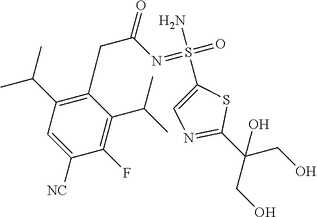 [SMILES: CC(C)c1cc(C#N)c(F)c(C(C)C)c1CC(=O)N=S(N)(=O)c1cnc(C(O)(CO)CO)s1]

### N-(amino(oxo)(2-(1,2,3-trihydroxypropan-2-yl)thiazol-5-yl)-λ⁶-sulfaneylidene)-2-(4-cyano-3-fluoro-2,6-diisopropylphenyl)acetamide (Scheme VII)

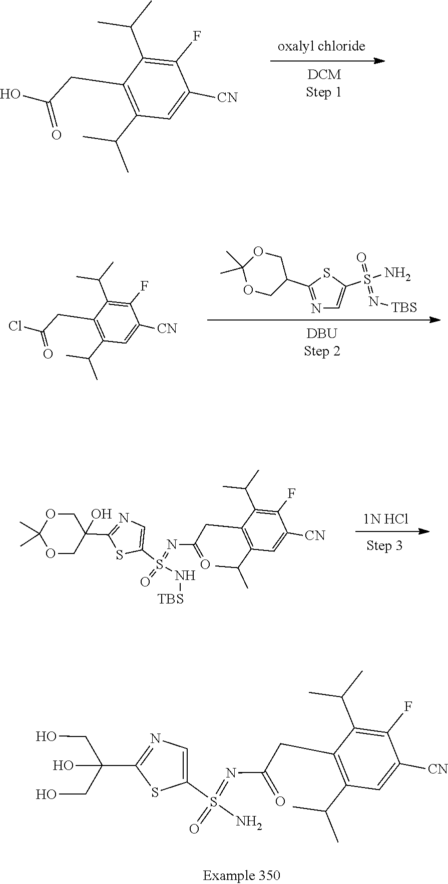

### Step 1: 2-(4-Cyano-3-fluoro-2,6-diisopropylphenyl)acetyl Chloride

To a stirred solution of 2-(4-cyano-3-fluoro-2,6-diisopropylphenyl)acetic acid (80 mg, 0.30 mmol) in DCM (6.0 mL) under nitrogen were added oxalyl chloride (383 mg, 3.0 mmol) and DMF (cat.) dropwise at 0° C. The reaction mixture was stirred for 2 h at RT. The reaction mixture was concentrated under vacuum. This resulted in 80 mg (crude) of the title product as brown oil.

### Step 2: N-(((tert-butyldimethylsilyl)amino)(2-(5-hydroxy-2,2-dimethyl-1,3-dioxan-5-yl)thiazol-5-yl) (oxo)-λ⁶-sulfaneylidene)-2-(4-cyano-3-fluoro-2,6-diisopropylphenyl)acetamide

To a stirred solution of N′-(tert-butyldimethylsilyl)-2-(5-hydroxy-2,2-dimethyl-1,3-dioxan-5-yl)thiazole-5-sulfonimidamide (100 mg, 0.25 mmol) in DCM (10 mL) were added DBU (75 mg, 0.49 mmol) dropwise at RT, followed by the addition of 2-(4-cyano-3-fluoro-2,6-diisopropylphenyl)acetyl chloride (80 mg, crude) in DCM (2 mL) dropwise at RT. The reaction solution was stirred for 1 h at RT. The reaction mixture was concentrated under vacuum. The residue was eluted from silica gel with DCM/MeOH (20:1). This resulted in 95 mg (48%, over two steps) of the title compound as light yellow oil. MS-ESI: 653 (M+1).

### Step 3: N-(amino(oxo)(2-(1,2,3-trihydroxypropan-2-yl)thiazol-5-yl)-λ⁶-sulfaneylidene)-2-(4-cyano-3-fluoro-2,6-diisopropylphenyl)acetamide

The mixture of N-(((tert-butyldimethylsilyl)amino)(2-(5-hydroxy-2,2-dimethyl-1,3-dioxan-5-yl)thiazol-5-yl) (oxo)-λ⁶-sulfaneylidene)-2-(4-cyano-3-fluoro-2,6-diisopropylphenyl)acetamide (95 mg, 0.15 mmol) in aq. HCl (1M, 3 mL) was stirred for 2 h at RT. The reaction mixture was concentrated under vacuum. The crude product was purified by prep-HPLC using the following conditions: Column, XSelect CSH Prep C18 OBD column, 5 um, 19*150 mm; Mobile Phase A: water (10 mM NH₄HCO₃), Mobile Phase B: MeOH, Gradient: 35% Phase B up to 50% over 10 min; Detector, UV 220 nm. This resulted in 35 mg (48%) of Example 350 as a white solid. MS-ESI: 499 (M+1). ¹H NMR (400 MHz, DMSO-d₆) δ 8.04 (s, 1H), 7.74 (br s, 2H), 7.60 (d, J=6.4 Hz, 1H), 5.91 (s, 1H), 4.80 (t, J=5.6 Hz, 2H), 3.82-3.71 (m, 2H), 3.69-3.59 (m, 4H), 3.18-3.07 (m, 2H), 1.21 (d, J=6.9 Hz, 3H), 1.19 (d, J=6.9 Hz, 3H), 1.13 (d, J=6.8 Hz, 3H), 1.11 (d, J=6.8 Hz, 3H).

### Example 351

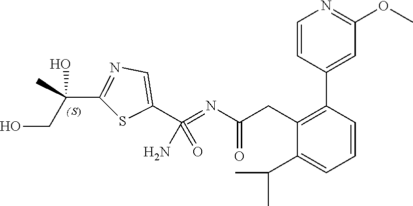

### N-(amino(2-((S or R)-1,2-dihydroxypropan-2-yl)thiazol-5-yl)(oxo)-λ⁶-sulfaneylidene)-2-(2-isopropyl-6-(2-methoxypyridin-4-yl)phenyl)acetamide (Scheme VIII)

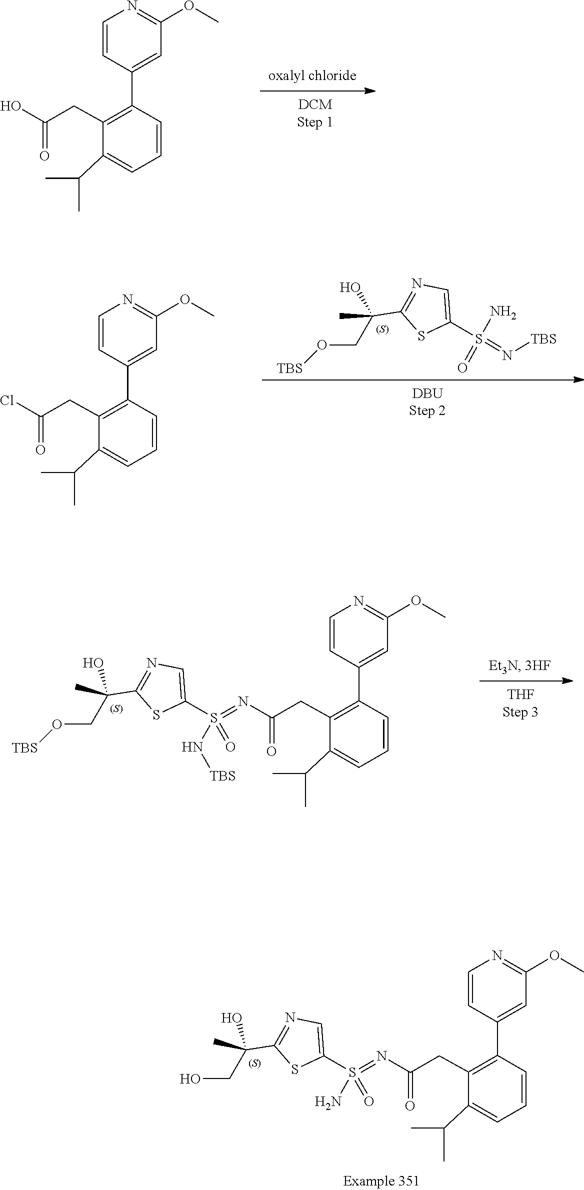

### Step 1: 2-(2-Isopropyl-6-(2-methoxypyridin-4-yl)phenyl)acetyl Chloride

To a stirred solution of 2-(2-isopropyl-6-(2-methoxypyridin-4-yl)phenyl)acetic acid (100 mg, 0.35 mmol) in DCM (5.0 mL) under nitrogen were added oxalyl chloride (53 mg, 0.42 mmol) and DMF (cat.) dropwise at 0° C. The reaction mixture was stirred for 1 h at RT. This resulted in 100 mg of the title compound (crude) as a yellow solid.

### Step 2: N-(((tert-butyldimethylsilyl)amino)(2-((S or R)-1-((tert-butyldimethylsilyl)oxy)-2-hydroxypropan-2-yl)thiazol-5-yl)(oxo)-λ⁶-sulfaneylidene)-2-(2-isopropyl-6-(2-methoxypyridin-4-yl)phenyl)acetamide

To a stirred solution of 2-(2-isopropyl-6-(2-methoxypyridin-4-yl)phenyl)acetyl chloride (100 mg, 0.33 mmol, crude from the last step) in THF (8.0 mL) were added DBU (150 mg, 0.99 mmol) dropwise at RT, followed by the addition of N′-(tert-butyldimethylsilyl)-2-((S or R)-1-((tert-butyldimethylsilyl)oxy)-2-hydroxypropan-2-yl)thiazole-5-sulfonimidamide (Intermediate 97′A, 153 mg, 0.33 mmol) in THF (3.0 mL) dropwise at RT. The reaction mixture was stirred for 3 h at RT. The reaction was quenched with water (10 mL) and the mixture was extracted with EtOAc (3×10 mL). The combined organic layers were dried over anhydrous Na₂SO₄ and concentrated under vacuum. The residue was purified by prep-TLC with DCM/MeOH (20:1). This resulted in 100 mg (38%, over two steps) of the title compound as yellow oil. MS-ESI: 733 (M+1).

### Step 3: N-(amino(2-((S or R)-1,2-dihydroxypropan-2-yl)thiazol-5-yl)(oxo)-λ⁶-sulfaneylidene)-2-(2-isopropyl-6-(2-methoxypyridin-4-yl)phenyl)acetamide

To a stirred solution of N-(((tert-butyldimethylsilyl)amino)(2-((S or R)-1-((tert-butyldimethylsilyl)oxy)-2-hydroxypropan-2-yl)thiazol-5-yl)(oxo)-λ⁶-sulfaneylidene)-2-(2-isopropyl-6-(2-methoxypyridin-4-yl)phenyl)acetamide (100 mg, 0.16 mmol) in THF (5.0 mL) was added Et₃N.3HF (102 mg, 0.63 mmol) in portions at RT. The reaction mixture was stirred overnight at RT. The reaction mixture was concentrated under vacuum. The residue was purified by prep-HPLC using the following conditions: XBridge Shield RP18 OBD column, 30*150 mm, 5 um; Mobile Phase A: water (10 mM NH₄HCO₃+0.1% NH₃.H₂O), Mobile Phase B: ACN; Flow rate: 60 mL/min; Gradient: 15% B to 38% B over 7 min; UV 254/210 nm; Rt: 7.18 min. This resulted in 11.9 mg (15%) of Example 351 as a white solid. MS-ESI: 505 (M+1). ¹H NMR (400 MHz, DMSO-d₆) some signals from diastereomers were resolved: δ 8.11 (d, J=5.2 Hz, 1H), 8.063 and 8.058 (s, 1H), 7.65 (br s, 2H), 7.33 (d, J=7.2 Hz, 1H), 7.27 (dd, J=7.6, 7.2 Hz, 1H), 6.999 and 6.996 (d, J=7.6 Hz, 1H), 6.82 (d, J=5.2 Hz, 1H), 6.72 (s, 1H), 6.14 and 6.13 (s, 1H), 4.99 (t, J=6.0 Hz, 1H), 3.87 (s, 3H), 3.54 (d, J=6.0 Hz, 2H), 3.52-3.36 (m, 2H), 2.99-2.91 (m, 1H), 1.44 (s, 3H), 1.12 (d, J=6.4 Hz, 3H), 1.05 and 1.04 (d, J=6.4 and 6.4 Hz, 3H).

### Example 352 and Example 353

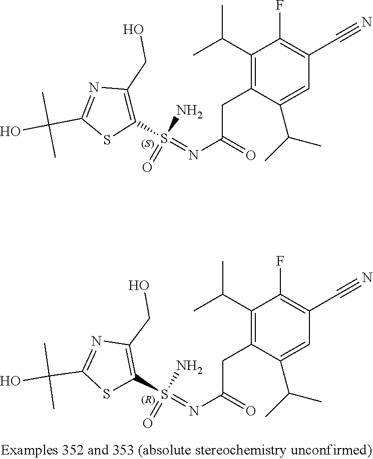

### (S) and (R)—N-(amino(4-(hydroxymethyl)-2-(2-hydroxypropan-2-yl)thiazol-5-yl)(oxo)-λ⁶-sulfaneylidene)-2-(4-cyano-3-fluoro-2,6-diisopropylphenyl)acetamide (Scheme X)

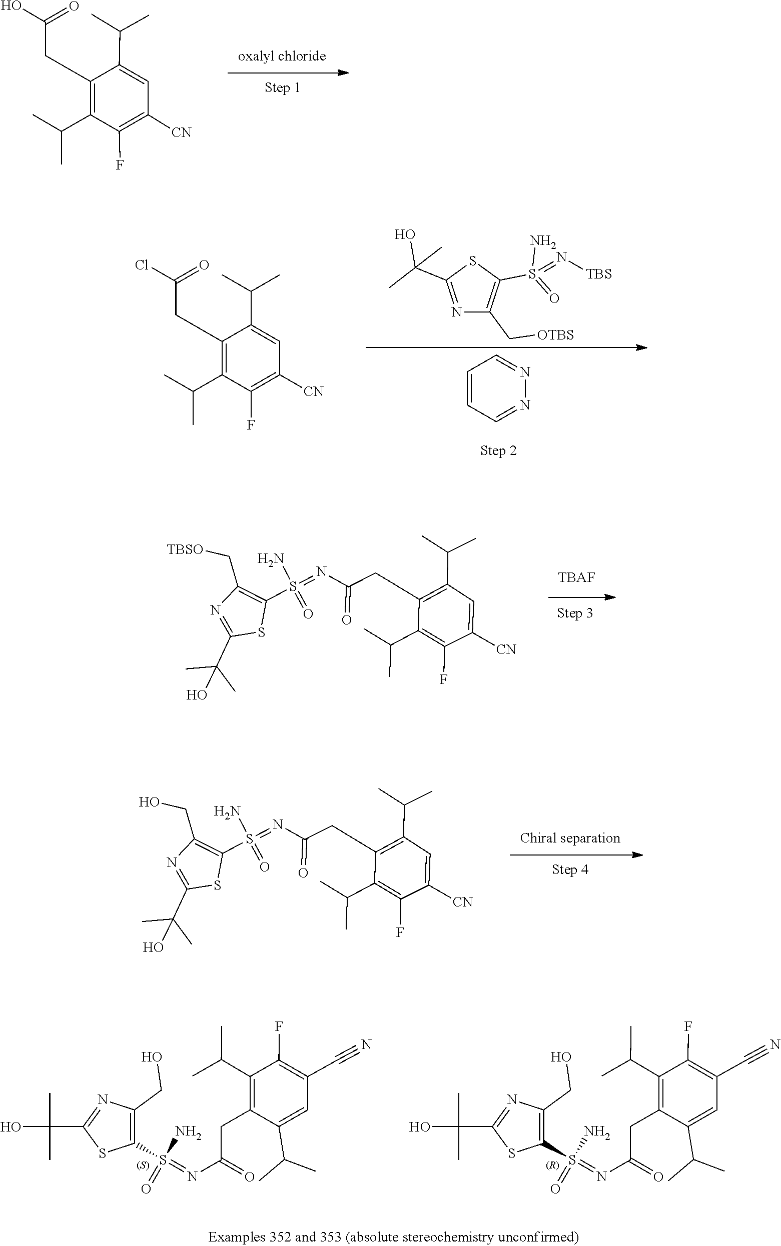

### Step 1: 2-(4-Cyano-3-fluoro-2,6-diisopropylphenyl)acetyl Chloride

To a stirred solution of 2-(4-cyano-3-fluoro-2,6-diisopropylphenyl)acetic acid (250 mg, 0.95 mmol) in DCM (5.0 mL) under nitrogen were added oxalyl chloride (241 mg, 1.9 mmol) and DMF (cat.) dropwise at 0° C. The reaction solution was stirred for 30 min at RT. The reaction solution was concentrated under vacuum. This resulted in 210 mg of the title compound (crude) as a yellow solid.

### Step 2: N-(amino(4-(((tert-butyldimethylsilyl)oxy)methyl)-2-(2-hydroxypropan-2-yl)thiazol-5-yl)(oxo)-λ⁶-sulfaneylidene)-2-(4-cyano-3-fluoro-2,6-diisopropylphenyl)acetamide

To a stirred solution of 2-(4-cyano-3-fluoro-2,6-diisopropylphenyl)acetyl chloride (210 mg, crude from the last step) in ACN (5.0 mL) was added pyridazine (60 mg, 0.75 mmol) dropwise at RT. The reaction solution was stirred for 20 min at RT. To the above solution was added N′-(tert-butyldimethylsilyl)-4-(((tert-butyl-dimethyl-silyl)oxy)methyl)-2-(2-hydroxypropan-2-yl)thiazole-5-sulfonimidamide (358 mg, 0.75 mmol) in portions at RT. The reaction mixture was stirred for 2 h at RT and concentrated under vacuum. The crude product was purified by Prep-TLC with EtOAc/PE (1:1). This resulted in 350 mg (60%, over two steps) of the title compound as yellow oil. MS-ESI: 611 (M+1).

### Step 3: N-(amino(4-(hydroxymethyl)-2-(2-hydroxypropan-2-yl)thiazol-5-yl)(oxo)-λ⁶-sulfaneylidene)-2-(4-cyano-3-fluoro-2,6-diisopropylphenyl)acetamide

To a stirred solution of N-(amino(4-(((tert-butyldimethylsilyl)oxy)methyl)-2-(2-hydroxypropan-2-yl)thiazol-5-yl)(oxo)-λ⁶-sulfaneylidene)-2-(4-cyano-3-fluoro-2,6-diisopropylphenyl)acetamide (350 mg, 0.57 mmol) in THF (5.0 mL) was added TBAF (300 mg, 1.15 mmol) in portions at RT. The reaction solution was stirred for 2 h at RT, then concentrated under vacuum. The residue was eluted from silica gel with EtOAc/PE (100:1). The resulting product (200 mg, crude) was purified by prep-HPLC using the following conditions: XBridge Shield RP18 OBD column, 19*250 mm, 10 um; mobile phase, water (10 mM NH₄HCO₃) and ACN (15% to 50% ACN over 7 min); Detector, UV 220/254 nm. This resulted in 100 mg (35%) of the title compound as a white solid. MS-ESI: 497 (M+1).

### Step 4: (S) and (R)—N-(amino(4-(hydroxymethyl)-2-(2-hydroxypropan-2-yl)thiazol-5-yl)(oxo)-λ⁶-sulfaneylidene)-2-(4-cyano-3-fluoro-2,6-diisopropylphenyl)acetamide

N-(amino(4-(hydroxymethyl)-2-(2-hydroxypropan-2-yl)thiazol-5-yl)(oxo)-λ⁶-sulfaneylidene)-2-(4-cyano-3-fluoro-2,6-diisopropylphenyl)acetamide (100 mg) was resolved by prep-chiral HPLC using the following conditions: CHIRALPAK IC, 2*25 cm, 5 um; Mobile Phase A: Hex (0.1% FA), Mobile Phase B: IPA; Flow rate: 20 mL/min; Gradient: 30% B to 30% B over 15.5 min; UV 220/254 nm; Rt₁: 6.384 min (Example 352); Rt₂: 11.194 min (Example 353). This resulted in 27.7 mg of Example 352 followed by 27.2 mg of Example 353, both as off-white solid.

Example 352: MS-ESI: 497 (M+1). ¹H NMR (400 MHz, DMSO-d₆) δ 8.11 (br s, 2H), 7.60 (d, J=6.4 Hz, 1H), 6.25 (s, 1H), 5.26 (br s, 1H), 4.63 (s, 2H), 3.88-3.65 (m, 2H), 3.17-3.06 (m, 2H), 1.48 (s, 3H), 1.47 (s, 3H), 1.22-1.07 (m, 12H).

Example 353: MS-ESI: 497 (M+1). ¹H NMR (400 MHz, DMSO-d₆) δ 8.04 (br s, 2H), 7.60 (d, J=6.4 Hz, 1H), 6.24 (s, 1H), 5.26 (br s, 1H), 4.63 (s, 2H), 3.88-3.65 (m, 2H), 3.15-3.05 (m, 2H), 1.47 (s, 3H), 1.46 (s, 3H), 1.22-1.07 (m, 12H).

### Example 354

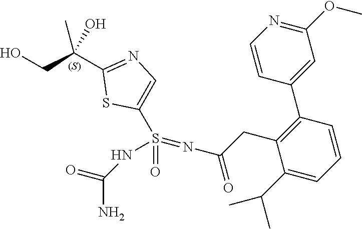

### N-((2-((S)-1,2-dihydroxypropan-2-yl)thiazol-5-yl)(oxo)(ureido)-λ⁶-sulfaneylidene)-2-(2-isopropyl-6-(2-methoxypyridin-4-yl)phenyl)acetamide (Scheme XI)

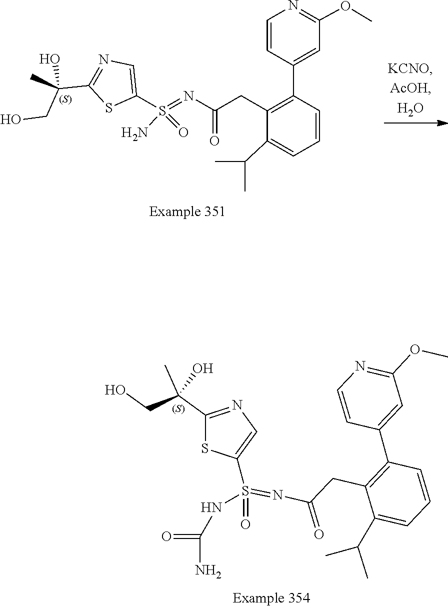

To a stirred solution of N-(amino(2-((S)-1,2-dihydroxypropan-2-yl)thiazol-5-yl)(oxo)-λ⁶-sulfaneylidene)-2-(2-isopropyl-6-(2-methoxypyridin-4-yl)phenyl)acetamide (Example 351, 110 mg, 0.218 mmol) in AcOH (6 mL) and water (0.6 mL) was added potassium cyanate (706 mg, 8.72 mmol) in portions at RT. The resulting mixture was stirred 16 h at 50° C. The reaction mixture was diluted with water (10 mL) and extracted with EtOAc (3×10 mL). The combined organic layers were dried over anhydrous Na₂SO₄ and concentrated under vacuum. The crude product was purified by Prep-HPLC with the following conditions (Column: XBridge Prep C18 OBD Column, 19×150 mm 5 um; Mobile Phase A: Water (10 mM NH₄HCO₃), Mobile Phase B: ACN; Flow rate: 25 mL/min; Gradient: 21% B to 26% B over 7 min; 210/254 nm; RT: 5.88 min). This resulted in 27.3 mg (22.9%) of Example 354 as a white solid. MS-ESI 548 (M+1). ¹H NMR (400 MHz, MeOH-d₄) δ 8.13-8.07 (m, 2H), 7.35 (d, J=7.9 Hz, 1H), 7.27 (t, J=7.8 Hz, 1H), 7.01 (d, J=7.6 Hz, 1H), 6.97-6.91 (m, 1H), 6.82 (s, 1H), 3.94 (s, 3H), 3.77 (d, J=11.2 Hz, 1H), 3.67 (d, J=11.4 Hz, 1H), 3.58 (s, 2H), 3.20-3.05 (m, 1H), 1.56 (s, 3H), 1.21 (d, J=6.8 Hz, 3H), 1.18 (d, J=6.8 Hz, 3H).

TABLE 41
Examples in the following table were prepared using similar conditions as described in
Example 354 and Scheme XI from appropriate starting material.

| | | | Exact |
|---|---|---|---|
| Ex. | | | Mass |
| # | Structure | IUPAC Name | [M + H]⁺ |
| 355 | 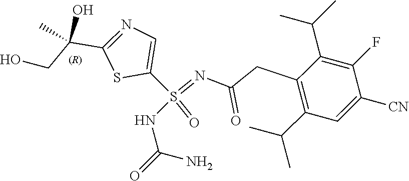 | 2-(4-cyano-3-fluoro-2,6-diisopropylphenyl)-N-((2-((R)-1,2- dihydroxypropan-2-yl)thiazol-5-yl)(oxo)(ureido)-λ⁶- sulfaneylidene)acetamide | 526 |

### Examples 356 and 357

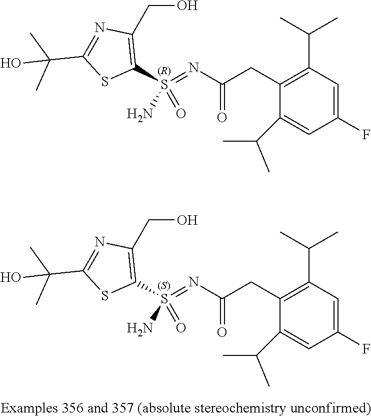

### (R) and (S)—N-(amino(4-(hydroxymethyl)-2-(2-hydroxypropan-2-yl)thiazol-5-yl)(oxo)-λ⁶-sulfaneylidene)-2-(4-fluoro-2,6-diisopropylphenyl)acetamide (Scheme XII)

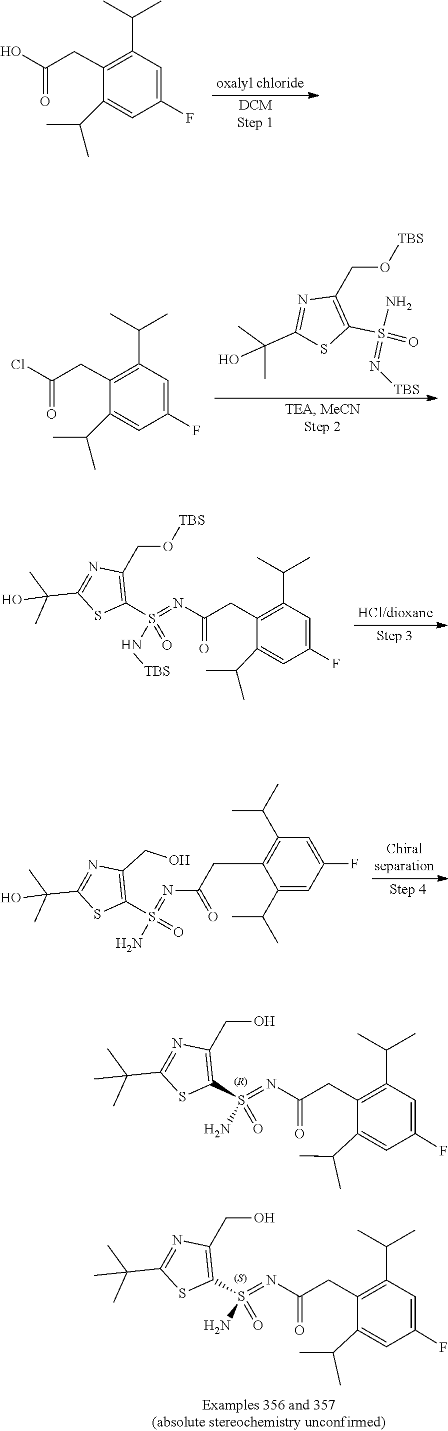

### Step 1: 2-(4-Fluoro-2,6-diisopropylphenyl)acetyl Chloride

To a stirred solution of 2-(4-fluoro-2,6-diisopropylphenyl)acetic acid (300 mg, 1.26 mmol) in DCM (10 mL) under nitrogen was added DMF (9.20 mg, 0.126 mmol) and oxalyl chloride (320 mg, 2.52 mmol) dropwise at 0° C. The resulting mixture was stirred for 1 h at RT. The resulting mixture was concentrated under vacuum. This resulted in the title compound which was used in the next step without further purification.

### Step 2: N-(((tert-butyldimethylsilyl)amino)(4-(((tert-butyldimethylsilyl)oxy)methyl)-2-(2-hydroxy-propan-2-yl)thiazol-5-yl)(oxo)-λ⁶-sulfaneylidene)-2-(4-fluoro-2,6-diisopropylphenyl)acetamide

To a stirred solution of N′-(tert-butyldimethylsilyl)-4-(((tert-butyldimethylsilyl)oxy)methyl)-2-(2-hydroxy-propan-2-yl)thiazole-5-sulfonimidamide (Intermediate 13, 604 mg, 1.26 mmol) in ACN (10 mL) was added TEA (202 mg, 2.00 mmol) at RT. To the above mixture was added 2-(4-fluoro-2,6-diisopropylphenyl)acetyl chloride (crude from last step) in DCM (2 mL) dropwise at 0° C. The resulting mixture was stirred for 4 h at RT. The reaction mixture was quenched with water (10 mL), then extracted with EtOAc (3×10 mL). The organic layers were combined and dried over anhydrous Na₂SO₄ and concentrated under vacuum. This resulted in the title compound (600 mg, 68.1% over 2 steps) as a yellow oil. MS-ESI: 700 (M+1).

### Step 3: N-(amino(4-(hydroxymethyl)-2-(2-hydroxypropan-2-yl)thiazol-5-yl)(oxo)-λ⁶-sulfaneylidene)-2-(4-fluoro-2,6-diisopropylphenyl)acetamide

To a stirred solution of N-(((tert-butyldimethylsilyl)amino)(4-(((tert-butyldimethylsilyl)oxy)methyl)-2-(2-hydroxypropan-2-yl)thiazol-5-yl)(oxo)-λ⁶-sulfaneylidene)-2-(4-fluoro-2,6-diisopropylphenyl)acetamide (100 mg, 14.3 mmol) in THF (5 mL) was added HCl in dioxane (4M, 5 mL, 20 mmol) dropwise at 0° C. The resulting mixture was stirred for 2 h at RT. The resulting mixture was concentrated under vacuum. The crude product was purified by Prep-HPLC with the following conditions (Column: Xselect CSH OBD Column 30*150 mm Sum; Mobile Phase A: Water (10 mM NH₄HCO₃+0.1% NH₃.H₂O), Mobile Phase B: ACN; Flow rate: 60 mL/min; Gradient: 10% B to 40% B over 7 min; 254/210 nm). This resulted in the title compound (50 mg 74.2%) as a white solid. MS-ESI: 472 (M+1).

### Step 4: Chiral Separation

N-(amino(4-(hydroxymethyl)-2-(2-hydroxypropan-2-yl)thiazol-5-yl)(oxo)-λ⁶-sulfaneylidene)-2-(4-fluoro-2,6-diisopropylphenyl)acetamide (300 mg) was purified by Prep-HPLC with the following conditions (Column: Chiralpak IC, 2*25 cm, 5 um; Mobile Phase A: Hex (0.1% FA), Mobile Phase B: EtOH; Flow rate: 20 mL/min; Gradient: 5% B to 5% B over 20 min; 220/254 nm; RT₁: 11.1 min, RT₂: 13 min. This resulted in Examples 356 (100 mg) and Examples 357 both as a white solid.

Examples 356 ¹H NMR (400 MHz, DMSO-d₆) δ 8.09 (br s, 2H), 6.85 (d, J=10.5 Hz, 2H), 6.24 (s, 1H), 5.17 (br s, 1H), 4.64 (d, J=3.0 Hz, 2H), 3.76-3.56 (m, 2H), 3.12-2.96 (m, 2H), 1.49 (s, 3H), 1.47 (s, 3H), 1.10 (d, J=6.8 Hz, 6H), 1.04 (d, J=6.7 Hz, 6H).

Examples 357 ¹H NMR (400 MHz, DMSO-d₆) δ 8.09 (br s, 2H), 6.85 (d, J=10.5 Hz, 2H), 6.24 (s, 1H), 5.17 (br s, 1H), 4.64 (d, J=3.0 Hz, 2H), 3.76-3.56 (m, 2H), 3.12-2.96 (m, 2H), 1.49 (s, 3H), 1.47 (s, 3H), 1.10 (d, J=6.8 Hz, 6H), 1.04 (d, J=6.7 Hz, 6H).

### Example 358

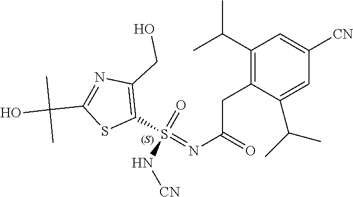

### (S)—N-(cyanamido(4-(hydroxymethyl)-2-(2-hydroxypropan-2-yl)thiazol-5-yl)(oxo)-λ⁶-sulfaneylidene)-2-(4-cyano-2,6-diisopropylphenyl)acetamide (Scheme V)

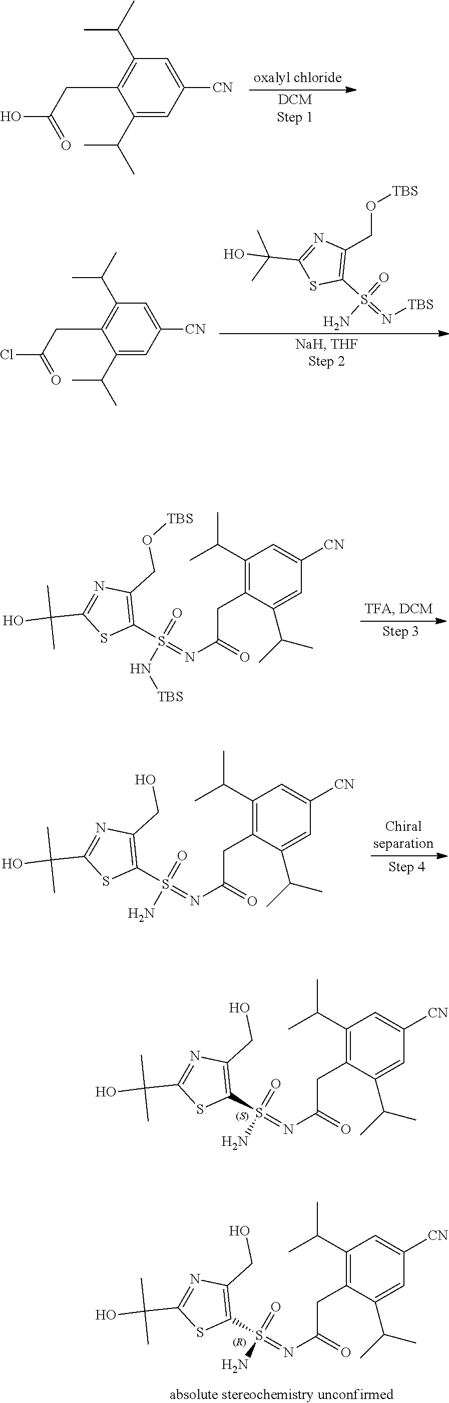

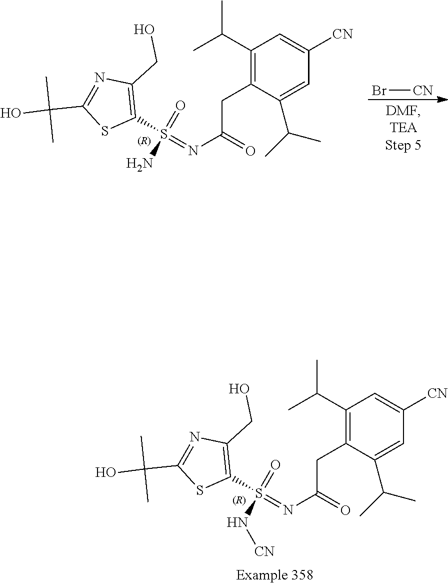

### Step 1: 2-(4-Cyano-2,6-diisopropylphenyl)acetyl Chloride

To a stirred solution of 2-(4-cyano-2,6-diisopropylphenyl)acetic acid (Intermediate 24, 76 mg, 0.31 mmol) in DCM (5 mL) was added DMF (4.5 mg, 0.062 mmol) at RT. This was followed by the addition of oxalyl chloride (394 mg, 3.1 mmol) dropwise with stirring at 0° C. The solution was stirred for 30 min at RT under nitrogen. The reaction mixture was concentrated under vacuum. This resulted in the title compound (crude) as a yellow oil which was used for next step without further purification.

### Step 2: N-(((tert-butyldimethylsilyl)amino)(4-(((tert-butyldimethylsilyl)oxy)methyl)-2-(2-hydroxypropan-2-yl)thiazol-5-yl)(oxo)-λ⁶-sulfaneylidene)-2-(4-cyano-2,6-diisopropylphenyl)acetamide

To a stirred solution of N′-(tert-butyldimethylsilyl)-4-(((tert-butyldimethylsilyl)oxy)methyl)-2-(2-hydroxypropan-2-yl)thiazole-5-sulfonimidamide (Intermediate 13, 144 mg, 0.30 mmol) in THF (3 mL) was added NaH (60% wt., 42 mg, 1.05 mmol) in portions at 0° C. The solution was stirred for 5 min at RT. Then to the above mixture was added a solution of 2-(4-cyano-2,6-diisopropylphenyl)acetyl chloride (crude from step 1) in THF (1 mL) dropwise at 0° C. The resulting reaction mixture was stirred for 2 h at RT, then quenched with 5 mL of water, and extracted with 2×5 mL of EtOAc. The organic layers were combined, dried over anhydrous Na₂SO₄, and concentrated under vacuum. This resulted in 202 mg (92% over 2 steps) of crude title compound as yellow oil. MS-ESI: 707 (M+1).

### Step 3: N-(amino(4-(hydroxymethyl)-2-(2-hydroxypropan-2-yl)thiazol-5-yl)(oxo)-λ⁶-sulfaneylidene)-2-(4-cyano-2,6-diisopropylphenyl)acetamide

To a stirred solution of N-(((tert-butyldimethylsilyl)amino)(4-(((tert-butyldimethylsilyl)oxy)methyl)-2-(2-hydroxypropan-2-yl)thiazol-5-yl)(oxo)-λ⁶-sulfaneylidene)-2-(4-cyano-2,6-diisopropylphenyl)acetamide (202 mg, 0.29 mmol) in DCM (10 mL) was added TFA (2 mL) dropwise at RT. The resulting reaction mixture was stirred for 2 h at RT. The resulting mixture was concentrated under vacuum. The crude product was purified by prep-HPLC using the following conditions: Column XBridge Shield RP18 OBD, 19*250 mm, 10 um; Mobile Phase A: Water (10 mM NH₄HCO₃), Mobile Phase B: ACN; Flow rate: 25 mL/min; Gradient: 8% B to 42% B over 8 min; Detector 254/210 nm; This resulted in 122 mg (89%) of the title compound (Example 22) as an off-white solid. MS-ESI: 479 (M+1).

### Step 4: Chiral Separation

N-(amino(4-(hydroxymethyl)-2-(2-hydroxypropan-2-yl)thiazol-5-yl)(oxo)-λ⁶-sulfaneylidene)-2-(4-cyano-2,6-diisopropylphenyl)acetamide (120 mg) was resolved by prep-HPLC using the following conditions: Column Chiralpak IC, 2*25 cm, 5 um; Mobile Phase A: Hex (0.1% FA), Mobile Phase B: EtOH; Flow rate: 20 mL/min; Gradient: 20% B to 20% B over 8.5 min; Detector 220/254 nm; RT₁: 4.5 min (Example 64); RT₂: 6.3 min (Example 65). This resulted in (48 mg Example 64) and (50 mg Example 65) both as a off-white solid. MS-ESI: 479 (M+1).

### Step 5: (S)—N-(cyanamido(4-(hydroxymethyl)-2-(2-hydroxypropan-2-yl)thiazol-5-yl)(oxo)-λ⁶-sulfaneylidene)-2-(4-cyano-2,6-diisopropylphenyl)acetamide

To a stirred solution of (R)—N-(amino(4-(hydroxymethyl)-2-(2-hydroxypropan-2-yl)thiazol-5-yl)(oxo)-λ⁶-sulfaneylidene)-2-(4-cyano-2,6-diisopropylphenyl)acetamide (Example 65, 47.9 mg, 0.10 mmol) in DMF (10 mL) was added TEA (40 mg, 0.40 mmol) dropwise at RT, followed by the addition of cyanogen bromide (21 mg, 0.20 mmol) in portions at RT. The reaction solution was stirred for 1 h at RT. The pH value of the solution was adjusted to 7 with NaOH (1 M). The mixture was concentrated under vacuum. The crude product was purified by prep-HPLC using the following conditions: Column XBridge Prep OBD C18, 30×150 mm Sum; Mobile Phase A: Water (10 mM NH₄HCO₃+0.1% NH₃.H₂O), Mobile Phase B: ACN; Flow rate: 60 mL/min; Gradient: 13% B to 43% B over 7 min; 210/254 nm; RT₁: 6.17 min. This resulted in 16 mg (31%) of Example 358 as a white solid. MS-ESI: 504 (M+1). ¹H NMR (400 MHz, MeOH-d₄) δ 7.40 (s, 2H), 4.95-4.70 (m, 2H), 3.84 (s, 2H), 3.25-3.10 (m, 2H), 1.58 (s, 3H), 1.56 (s, 3H), 1.18 (d, J=6.8 Hz, 6H), 1.15 (d, J=6.8 Hz, 6H).

### Example 359

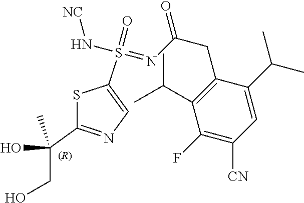

### N-(cyanamido(2-((R or S)-1,2-dihydroxypropan-2-yl)thiazol-5-yl)(oxo)-λ⁶-sulfaneylidene)-2-(4-cyano-3-fluoro-2,6-diisopropylphenyl)acetamide (Scheme IX)

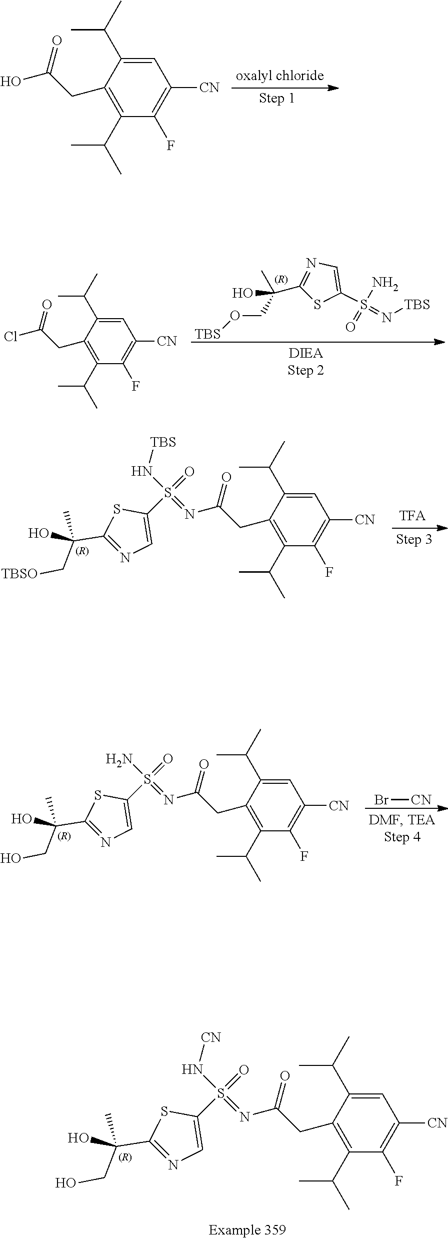

### Step 1: 2-(4-Cyano-3-fluoro-2,6-diisopropylphenyl)acetyl Chloride

To a stirred solution of 2-(4-cyano-3-fluoro-2,6-diisopropylphenyl)acetic acid (200 mg, 0.76 mmol) in DCM (8.0 mL) under nitrogen were added DMF (cat.) and oxalyl chloride (116 mg, 0.91 mmol) dropwise at 0° C. The reaction solution was stirred for 1 h at RT. The reaction solution was concentrated under vacuum. This resulted in 200 mg of the title compound (crude) as a brown solid.

### Step 2: N-(((tert-butyldimethylsilyl)amino)(2-((R or S)-1-((tert-butyldimethylsilyl)oxy)-2-hydroxy-propan-2-yl)thiazol-5-yl)(oxo)-λ⁶-sulfaneylidene)-2-(4-cyano-3-fluoro-2,6-diisopropylphenyl)acetamide

To a stirred solution of 2-(4-cyano-3-fluoro-2,6-diisopropylphenyl)acetyl chloride (200 mg, crude) in THF (10 mL) were added N′-(tert-butyldimethylsilyl)-2-((R or S)-1-((tert-butyldimethylsilyl)oxy)-2-hydroxy-propan-2-yl)thiazole-5-sulfonimidamide (Intermediate 97′B, 375 mg, 0.81 mmol) and DIEA (130 mg, 1.01 mmol) at RT. The reaction solution was stirred for 1 h at RT. The reaction solution was concentrated under vacuum. The residue was eluted from silica gel with EtOAc/PE (1:7). This resulted in 410 mg (76%, over two steps) of the title compound as a yellow solid. MS-ESI: 711 (M+1).

### Step 3: N-(amino(2-((R or S)-1,2-dihydroxypropan-2-yl)thiazol-5-yl)(oxo)-λ⁶-sulfaneylidene)-2-(4-cyano-3-fluoro-2,6-diisopropylphenyl)acetamide

To a stirred solution of N-(((tert-butyldimethylsilyl)amino)(2-((R or S)-1-((tert-butyldimethylsilyl)oxy)-2-hydroxypropan-2-yl)thiazol-5-yl)(oxo)-λ⁶-sulfaneylidene)-2-(4-cyano-3-fluoro-2,6-diisopropylphenyl)acetamide (410 mg, 0.58 mmol) in DCM (15 mL) was added TFA (6.57 mg, 0.058 mmol) in DCM (0.5 mL) dropwise at RT. The reaction solution was stirred for 2 h at RT. The pH value of the solution was adjusted to 7 with NH₄HCO₃ (1 M). The mixture was concentrated under vacuum. The residue was eluted from silica gel with EtOAc/PE (1:5). This resulted in 250 mg (89%) of the title compound as a white solid. MS-ESI: 483 (M+1).

### Step 4: N-(cyanamido(2-((R or S)-1,2-dihydroxypropan-2-yl)thiazol-5-yl)(oxo)-λ⁶-sulfaneylidene)-2-(4-cyano-3-fluoro-2,6-diisopropylphenyl)acetamide

To a stirred solution of N-(amino(2-((R or S)-1,2-dihydroxypropan-2-yl)thiazol-5-yl)(oxo)-λ⁶-sulfaneylidene)-2-(4-cyano-3-fluoro-2,6-diisopropylphenyl)acetamide (200 mg, 0.41 mmol) in DMF (10 mL) was added TEA (168 mg, 1.66 mmol) dropwise at RT, followed by the addition of cyanogen bromide (88 mg, 0.83 mmol) at RT. The reaction solution was stirred for 1 h at RT. The pH value of the solution was adjusted to 7 with NaOH (1 M). The mixture was concentrated under vacuum. The crude product was purified by prep-HPLC using the following conditions: XBridge Shield RP18 OBD column, 19*250 mm, 10 um; mobile phase A: water (10 mM NH₄HCO₃) and mobile phase B: ACN (0.1% DEA); gradient: 31% Phase B up to 34% over 8 min); Detector, UV 254 nm. This resulted in 63 mg (30%) of Example 359 as a white solid. MS-ESI: 508 (M+1). ¹H NMR (300 MHz, MeOD-d₄) δ 8.08 (s, 1H), 7.41 (d, J=6.3 Hz, 1H), 3.83 (s, 2H), 3.72-3.63 (m, 2H), 3.23-3.17 (m, 2H), 1.53 and 1.52 (s, 3H), 1.35-1.10 (m, 12H).

TABLE 42
Examples in the following table were prepared using similar conditions as described in
Example 359 and Scheme V from appropriate starting materials. The IUPAC Names of the
below structures were generated using PerkinElmer ChemDraw, version: 16.0.0.82 (68).

| | | | Exact |
|---|---|---|---|
| | | | Mass |
| Ex. # | Structure | IUPAC Name | [M + H]⁺ |
| 360 | 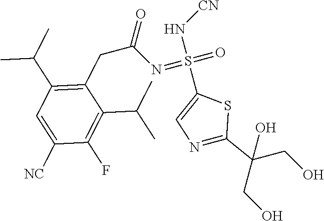 [SMILES: CC(C)c1cc(C#N)c(F)c(C(C)C)c1CC(=O)N=S(=O)(NC#N)c1cnc(C(O)(CO)CO)s1] | N-(cyanamido(oxo)(2-(1,2,3-trihydroxypropan-2-yl)thiazol- 5-yl)-λ⁶-sulfaneylidene)-2-(4-cyano-3-fluoro-2,6- diisopropylphenyl)acetamide (Prepared from Example 350) | 524 |
| 361 | 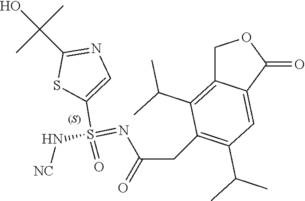 | (S)-N-(cyanamido(2-(2-hydroxypropan-2-yl)thiazol-5-yl) (oxo)-λ⁶-sulfaneylidene)-2-(4,6-diisopropyl-1-oxo-1,3- dihydroisobenzofuran-5-yl)acetamide (Prepared from Example 318) | 505 |
| 362 | 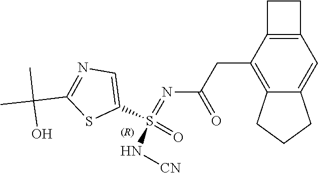 | (R)-N-(cyanamido(2-(2-hydroxypropan-2-yl)thiazol-5-yl) (oxo)-λ⁶-sulfaneylidene)-2-(2,4,5,6-tetrahydro-1H-cyclobuta [f]inden-3-yl)acetamide (Prepared from Example 223) | 431 |
| 363 | 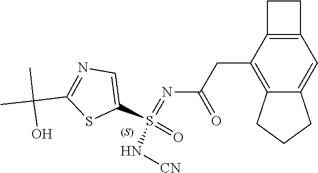 | (S)-N-(cyanamido(2-(2-hydroxypropan-2-yl)thiazol-5-yl) (oxo)-λ⁶-sulfaneylidene)-2-(2,4,5,6-tetrahydro-1H-cyclobuta [f]inden-3-yl)acetamide (Prepared from Example 224) | 431 |
| 364 | 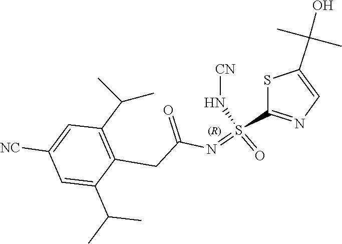 | (R)-N-(cyanamido(5-(2-hydroxypropan-2-yl)thiazol-2-yl) (oxo)-λ⁶-sulfaneylidene)-2-(4-cyano-2,6-diisopropylphenyl) acetamide (Prepared from Example 82) | 474 |
| 365 | 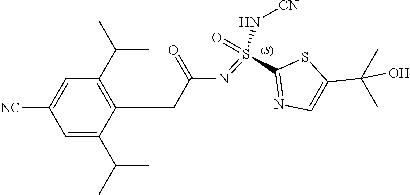 | (S)-N-(cyanamido(5-(2-hydoxypropan-2-yl)thiazol-2-yl) (oxo-sulfaneylidene)-2-(4-cyano-2,6-diisopropylphenyl) acetamide (Prepared from Example 83) | 474 |
| 366 | 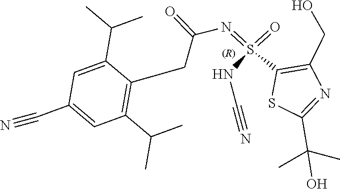 | (R)-N-(cyanamido(4-(hydroxymethyl)-2-(2-hydroxypropan- 2-yl)thiazol-5-yl)(oxo)-λ⁶-sulfaneylidene)-2-(4-cyano- 2,6-diisopropylphenyl)acetamide (Prepared from Example 64) | 504 |
| 367 | 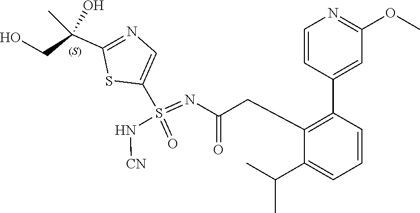 | N-(cyanamido(2-((S)-1,2-dihydroxypropan-2-yl)thiazol- 5-yl)(oxo)-λ⁶-sulfaneylidene)-2-(2-isopropyl-6-(2- methoxypyridin-4-yl)phenyl)acetamide (Prepared from Example 351) | 530 |
| 368 | 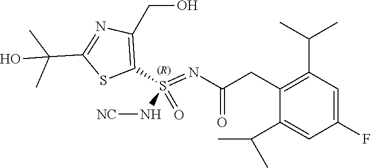 | (R)-N-(cyanamido(4-(hydroxymethyl)-2-(2-hydroxypropan- 2-yl)thiazol-5-yl)(oxo)-λ⁶-sulfaneylidene)-2-(4-fluoro- 2,6-diisopropylphenyl)acetamide (Prepared from Example 357) | 497 |
| 369 | 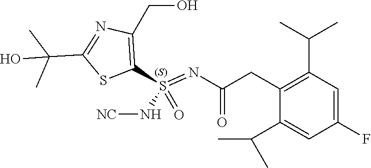 | (S)-N-(cyanamido(4-(hydroxymethyl)-2-(2-hydroxypropan- 2-yl)thiazol-5-yl)(oxo)-λ⁶-sulfaneylidene)-2-(4-fluoro- 2,6-diisopropylphenyl)acetamide (Prepared from Example 356) | 497 |
| 370 | 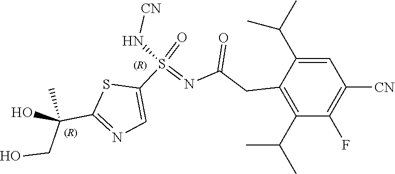 | N-((R)-cyanamido(2-((R)-1,2-dihydroxypropan-2-yl)thiazol- 5-yl)(oxo)-λ⁶-sulfaneylidene)-2-(4-cyano-3-fluoro-2,6- diisopropylphenyl)acetamide (Prepared from Intermediate 15 and chiral Intermediate 97′B) | 508 |
| 371 | 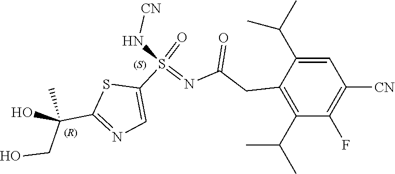 | N-((S)-cyanamido(2-((R)-1,2-dihydroxypropan-2-yl)thiazol- 5-yl)(oxo)-λ⁶-sulfaneylidene)-2-(4-cyano-3-fluoro-2,6- diisopropylphenyl)acetamide (Prepared from Intermediate 15 and chiral Intermediate 97′B) | 508 |
| 372 | 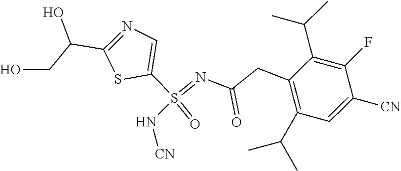 [SMILES: CC(C)c1cc(C#N)c(F)c(C(C)C)c1CC(=O)N=S(=O)(NC#N)c1cnc(C(O)CO)s1] | N-(cyanamido(2-(1,2-dihydroxyethyl)thiazol-5-yl)(oxo)-λ⁶- sulfaneylidene)-2-(4-cyano-3-fluoro-2,6- diisopropylphenyl)acetamide | 494 |
| 373 | 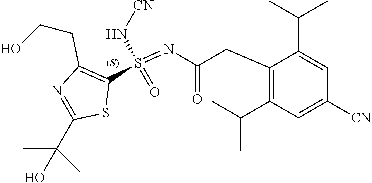 | (S)-N-(cyanamido(4-(2-hydroxyethyl)-2-(2-hydroxypropan- 2-yl)thiazol-5-yl)(oxo)-λ⁶-sulfaneylidene)-2-(4-cyano-2,6- diisopropylphenyl)acetamide | 518 |
| 374 | 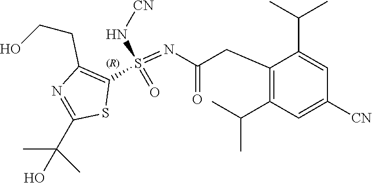 | (R)-N-(cyanamido(4-(2-hydroxyethyl)-2-(2-hydroxypropan- 2-yl)thiazol-5-yl)(oxo)-λ⁶-sulfaneylidene)-2-(4-cyano-2,6- diisopropylphenyl)acetamide | 518 |
| 375 | 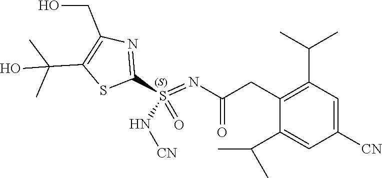 | (S)-N-(cyanamido(4-(hydroxymethyl)-5-(2-hydroxypropan- 2-yl)thiazol-2-yl)(oxo)-λ⁶-sulfaneylidene)-2-(4-cyano-2,6- diisopropylphenyl)acetamide | 504 |
| 376 | 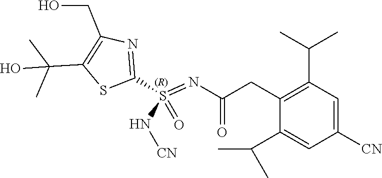 | (R)-N-(cyanamido(4-(hydroxymethyl)-5-(2-hydroxypropan- 2-yl)thiazol-2-yl)(oxo)-λ⁶-sulfaneylidene)-2-(4-cyano-2,6- diisopropylphenyl)acetamide | 504 |
| 377 | 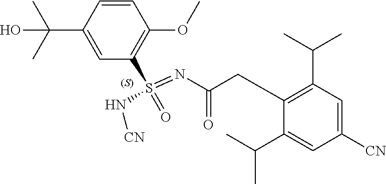 | (S)-N-(cyanamido(5-(2-hydroxypropan-2-yl)-2- methoxyphenyl)(oxo)-λ⁶-sulfaneylidene)-2-(4-cyano-2,6- diisopropylphenyl)acetamide | 497 |
| 378 | 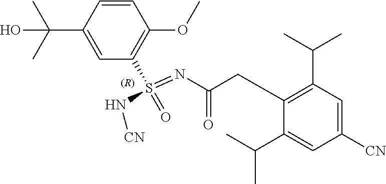 | (R)-N-(cyanamido(5-(2-hydroxypropan-2-yl)-2- methoxyphenyl)(oxo)-λ⁶-sylfaneylidene)-2-(4-cyano-2,6- diisopropylphenyl)acetamide | 497 |

### Example 379

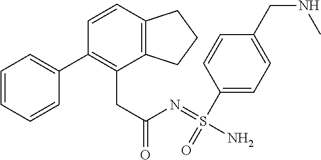 [SMILES: CNCc1ccc(S(N)(=O)=NC(=O)Cc2c(-c3ccccc3)ccc3c2CCC3)cc1]

### N-(amino(4-((methylamino)methyl)phenyl)(oxo)-λ⁶-sulfaneylidene)-2-(5-phenyl-2,3-dihydro-1H-inden-4-yl)acetamide (Scheme VI)

### Example 380 and Example 381

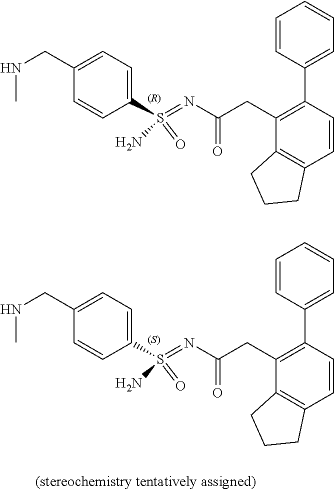

### (R) and (S)—N-(amino(4-((methylamino)methyl)phenyl)(oxo)-λ⁶-sulfaneylidene)-2-(5-phenyl-2,3-dihydro-1H-inden-4-yl)acetamide (Scheme VI)

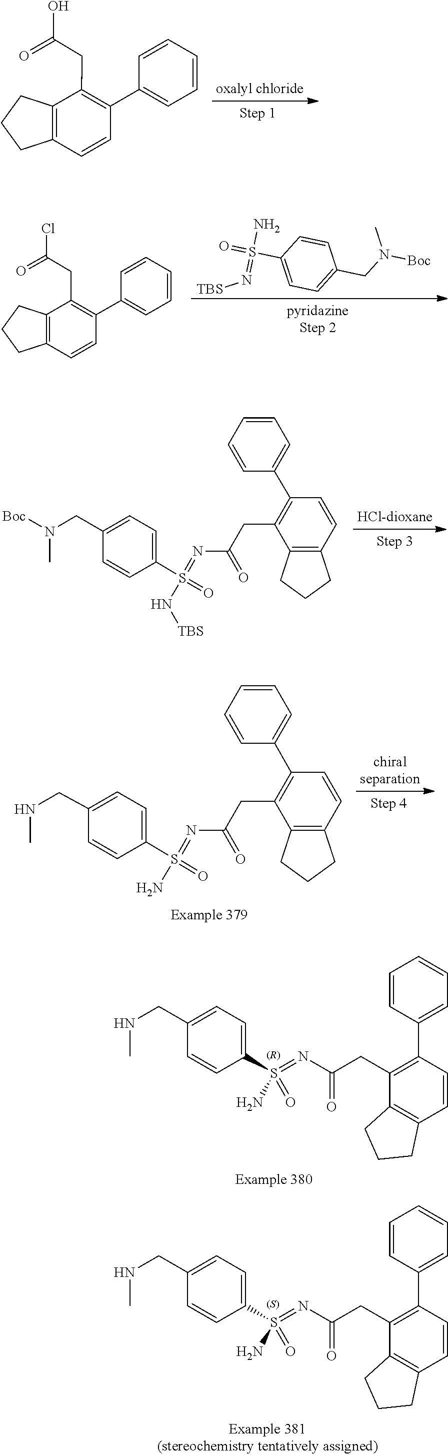

### Step 1: 2-(5-Phenyl-2,3-dihydro-1H-inden-4-yl)acetyl Chloride

To a stirred solution of 2-(5-phenyl-2,3-dihydro-1H-inden-4-yl)acetic acid (80 mg, 0.317 mmol) in DCM (10 mL) under nitrogen was added DMF (cat.), followed by oxalyl chloride (80.5 mg, 0.634 mmol) dropwise at 0° C. The resulting solution was stirred for 16 h at RT. The reaction mixture was concentrated under vacuum. This resulted in the title compound which was used for next step directly.

### Step 2: Tert-butyl (4-(N-(tert-butyldimethylsilyl)-N′-(2-(5-phenyl-2,3-dihydro-1H-inden-4-yl)acetyl)-sulfamidimidoyl)benzyl)(methyl)carbamate

To a stirred solution of 2-(5-phenyl-2,3-dihydro-1H-inden-4-yl)acetyl chloride (crude from last step) in ACN (10 mL) was added pyridazine (38.1 mg, 0.476 mmol) dropwise at 0° C., followed by tert-butyl (4-(N′-(tert-butyldimethylsilyl)sulfamidimidoyl)benzyl)(methyl)carbamate (131 mg, 0.317 mmol) in ACN (3 mL) dropwise at 0° C. The resulting mixture was stirred for 16 h at RT and then quenched with water (10 mL) and extracted with EtOAc (3×100 mL). The organic layers were combined, dried over anhydrous Na₂SO₄, and concentrated under vacuum. The residue was purified by Prep-TLC (PE/EtOAc=1:1) to afford the title compound (100 mg, 48.7% over 2 steps) as a yellow oil. MS-ESI: 648 (M+1).

### Step 3: N-(amino(4-((methylamino)methyl)phenyl)(oxo)-λ⁶-sulfaneylidene)-2-(5-phenyl-2,3-dihydro-1H-inden-4-yl)acetamide

To a stirred solution of tert-butyl (4-(N-(tert-butyldimethylsilyl)-N′-(2-(5-phenyl-2,3-dihydro-1H-inden-4-yl)acetyl)sulfamidimidoyl)benzyl)(methyl)carbamate (100 mg, 0.154 mmol) in dioxane (5 mL) was added HCl-dioxane (4 M, 5 mL) dropwise at 0° C. The resulting solution was stirred for 16 h at RT, then concentrated under vacuum. The residue was purified by Prep-HPLC using the following conditions: XBridge Prep C18 OBD column, 19×150 mm 5 um; mobile phase, water (10 mM NH₄HCO₃) and ACN (29% Phase B up to 51% over 7 min); Detector, UV 254 nm/220 nm. This resulted in 57 mg (85%) of Example 379 as a yellow solid. MS-ESI: 434 (M+1).

### Step 4: Chiral Separation

The product 50 mg of N-(amino(4-((methylamino)methyl)phenyl)(oxo)-λ⁶-sulfaneylidene)-2-(5-phenyl-2,3-dihydro-1H-inden-4-yl)acetamide was resolved by the follow condition: Column: CHIRALPAK IH, 2.0*25 cm, 5 um; Mobile Phase A: Hex:DCM=3:1 (10 mM NH₃-MeOH), Mobile Phase B: IPA; Flow rate: 20 mL/min; Gradient: 10% B to 10% B over 20 min; 220/254 nm; RT₁: 7.0 min (Example 380); RT₂: 15.9 min (Example 381); This resulted in 15 mg of Example 380 and 16 mg of Example 381. MS-ESI: both 434 (M+1).

Example 380 ¹H NMR (300 MHz, DMSO-d₆) δ 7.83 (d, J=7.9 Hz, 2H), 7.60 (d, J=8.0 Hz, 2H), 7.46-7.30 (m, 3H), 7.22-7.08 (m, 3H), 6.95 (d, J=7.6 Hz, 1H), 3.88 (s, 2H), 3.41 (s, 2H), 2.89 (t, J=7.5 Hz, 2H), 2.80-2.60 (m, 2H), 2.35 (s, 3H), 2.10-1.85 (m, 2H).

Example 381 ¹H NMR (300 MHz, DMSO-d₆) δ 7.82 (d, J=7.9 Hz, 2H), 7.59 (d, J=8.0 Hz, 2H), 7.46-7.30 (m, 3H), 7.23-7.09 (m, 3H), 6.95 (d, J=7.6 Hz, 1H), 3.86 (s, 2H), 3.41 (s, 2H), 2.89 (t, J=7.6 Hz, 2H), 2.80-2.60 (m, 2H), 2.34 (s, 3H), 2.10-1.85 (m, 2H).

TABLE 43
Examples in the following table were prepared using similar conditions as described in
Example 379 and Scheme VI from appropriate starting material.

| | | | Exact |
|---|---|---|---|
| | | | Mass |
| Ex. # | Structure | IUPAC Name | [M + H]⁺ |
| 382 | 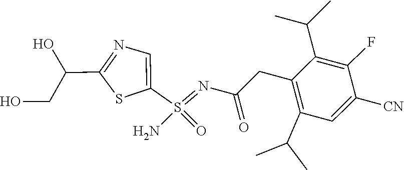 [SMILES: CC(C)c1cc(C#N)c(F)c(C(C)C)c1CC(=O)N=S(N)(=O)c1cnc(C(O)CO)s1] | N-(amino(2-(1,2-dihydroxyethyl)thiazol-5-yl)(oxo)-λ⁶- sulfaneylidene)-2-(4-cyano-3-fluoro-2,6- diisopropylphenyl)acetamide | 469 |

TABLE 44
Examples in the following table were prepared using similar conditions as described in
Example 380 and Example 381 and Scheme VI from appropriate starting materials. For the
chiral resolution, the faster-eluting enantiomer is always listed first in the table followed by the
slower-eluting enantiomer of the pair. The sulfur stereocenters were tentatively assigned for
registration purpose based on relative potency of enantiomers.

| | | | | | LC-MS |
|---|---|---|---|---|---|
| Ex. # | Structure | IUPAC Name | Column | Eluents | [M + H]⁺ |
| 383 | 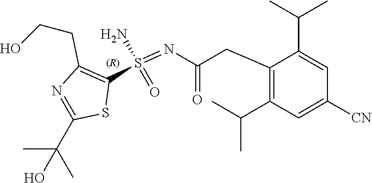 | (R) or (S)-N-(amino(4-(2-hydroxyethyl)-2- (2-hydroxypropan-2-yl)thiazol-5-yl)(oxo)- λ⁶-sulfaneylidene)-2-(4-cyano-2,6- diisopropylphenyl)acetamide | Chiralpak IC, 2 * 25 cm, 5 um | 20% EtOH in Hex (0.1% FA) | 493 |
| 384 | 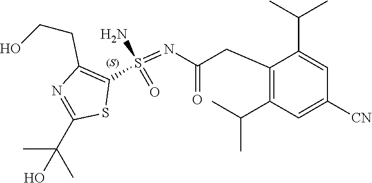 | (S) or (R)-N-(amino(4-(2-hydroxyethyl)-2- (2-hydroxypropan-2-yl)thiazol-5-yl)(oxo)- λ⁶-sulfaneylidene)-2-(4-cyano-2,6- diisopropylphenyl)acetamide | Chiralpak IC, 2 * 25 cm, 5 um | 20% EtOH in Hex (0.1% FA) | 493 |
| 385 | 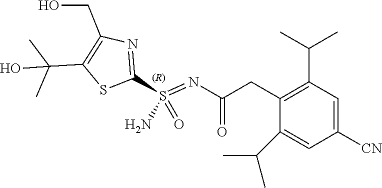 | (R) or (S)-N-(amino(4-(hydroxymethyl)-5- (2-hydroxypropan-2-yl)thiazol-2-yl)(oxo)- λ⁶-sulfaneylidene)-2-(4-cyano-2,6- diisopropylphenyl)acetamide | Chiralpak IC, 2 * 25 cm, 5 um | 15% EtOH in Hex (0.1% FA) | 479 |
| 386 | 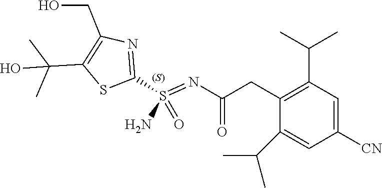 | (S) or (R)-N-(amino(4-(hydroxymethyl)-5- (2-hydroxypropan-2-yl)thiazol-2-yl)(oxo)- λ⁶-sulfaneylidene)-2-(4-cyano-2,6- diisopropylphenyl)acetamide | Chiralpak IC, 2 * 25 cm, 5 um | 15% EtOH in Hex (0.1% FA) | 479 |

### Example 387 and Example 388

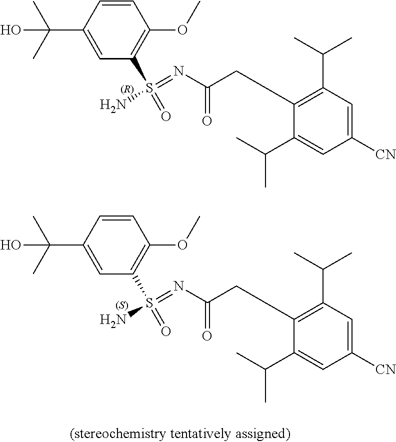

### (R) and (S)—N-(amino(5-(2-hydroxypropan-2-yl)-2-methoxyphenyl)(oxo)-λ⁶-sulfaneylidene)-2-(4-cyano-2,6-diisopropylphenyl)acetamide (Scheme 2)

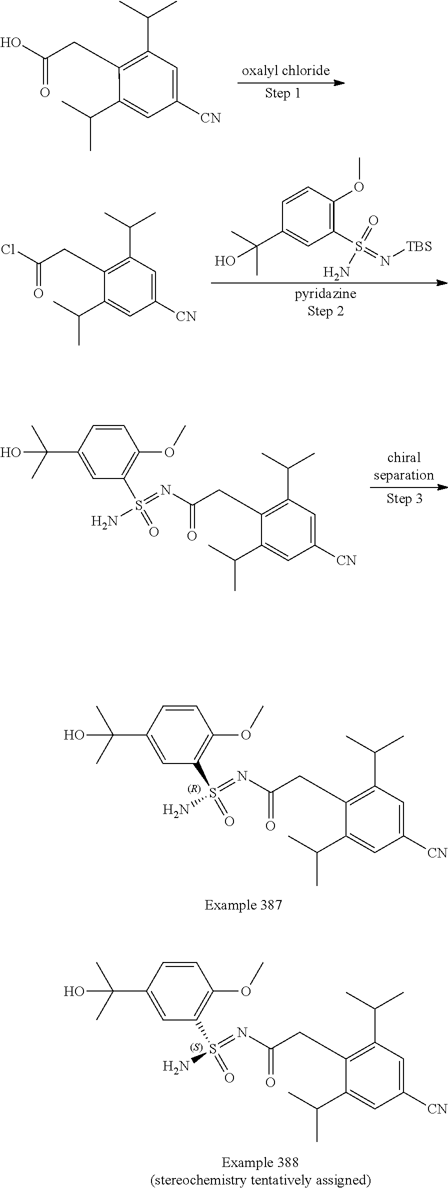

Step 1 used identical procedure as Example 358's step 1.

### Step 2: N-(amino(5-(2-hydroxypropan-2-yl)-2-methoxyphenyl)(oxo)-λ⁶-sulfaneylidene)-2-(4-cyano-2,6-diisopropylphenyl)acetamide

To a stirred solution of N′-(tert-butyldimethylsilyl)-5-(2-hydroxypropan-2-yl)-2-methoxybenzene-sulfonimidamide (200 mg, 0.557 mmol) in ACN (10 mL) under nitrogen was added pyridazine (89 mg, 1.11 mmol) at RT, followed by 2-(4-cyano-2,6-diisopropylphenyl)acetyl chloride (crude prepared from last step) in ACN (10 mL) dropwise at 0° C. The resulting solution was stirred for 2 h at RT. The resulting mixture was concentrated under vacuum and purified by Prep-HPLC with the following conditions: Column, Sunfire prep C18 column, 30*150, 5 um; mobile phase, Water (0.05% FA) and ACN (37% ACN up to 51% over 10 min); Detector, UV 254 nm. This resulted in 100 mg (38.0% over 2 steps) of the title compound as a white solid. MS-ESI: 472 (M+1).

### Step 3: Chiral Separation

The product from last step (100 mg) was resolved by Chiral-Prep-HPLC with the following conditions: Column, Chiralpak IC, 2*25 cm, 5 um; mobile phase, Hex (0.1% FA) and EtOH (hold 30% EtOH over 9 min); Detector, UV 254 nm. This resulted in 3.8 mg of Example 387 and 4.3 mg of Example 388 both as a white solid. MS-ESI: both 472 (M+1).

Example 387 ¹H NMR (400 MHz, DMSO-d₆) δ 7.86 (d, J=2.3 Hz, 1H), 7.61 (dd, J=8.7, 2.3 Hz, 1H), 7.48 (s, 2H), 7.35 (s, 2H), 7.13 (d, J=8.7 Hz, 1H), 5.13 (s, 1H), 3.86 (s, 3H), 3.78-3.65 (m, 2H), 3.12-2.97 (m, 2H), 1.383 (s, 3H), 1.376 (s, 3H), 1.12 (d, J=6.8 Hz, 6H), 1.05 (d, J=6.7 Hz, 6H).

Example 388 ¹H NMR (400 MHz, DMSO-d₆) δ 7.86 (d, J=2.4 Hz, 1H), 7.61 (dd, J=8.7, 2.4 Hz, 1H), 7.49 (s, 2H), 7.35 (s, 2H), 7.13 (d, J=8.7 Hz, 1H), 5.13 (s, 1H), 3.86 (s, 3H), 3.79-3.65 (m, 2H), 3.11-3.00 (m, 2H), 1.383 (s, 3H), 1.376 (s, 3H), 1.12 (d, J=6.8 Hz, 6H), 1.05 (d, J=6.8 Hz, 6H).

TABLE 43
Examples in the following table were prepared using similar conditions as described in
Example 93 and Scheme 1B from appropriate starting materials.

| | | | Exact Mass |
|---|---|---|---|
| Ex. # | Structure | IUPAC Name | [M + H]⁺ |
| 389 | 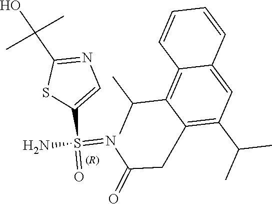 | (R)-N-(amino(2-(2-hydroxypropan-2-yl)thiazol-5-yl) (oxo)-λ⁶-sulfaneylidene)-2-(1,3- diisopropylnaphthalen-2-yl)acetamide | 474 |
| 390 | 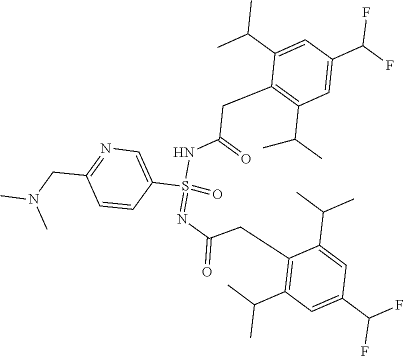 [SMILES: CC(C)c1cc(C(F)F)cc(C(C)C)c1CC(=O)N=S(=O)(NC(=O)Cc1c(C(C)C)cc(C(F)F)cc1C(C)C)c1ccc(CN(C)C)nc1] | 2-(4-(Difluoromethyl)-2,6-diisopropylphenyl)-N-((2-(4- (difluoromethyl)-2,6-diisopropylphenyl)acetamido)(6- ((dimethylamino)methyl)pyridin-3-yl)(oxo)-λ⁶- sulfaneylidene)acetamide | 719 |

TABLE 45
Examples in the following table were prepared using similar conditions as described in
Example 4 and Scheme 2 from appropriate starting materials.

| | | | Exact |
|---|---|---|---|
| | | | Mass |
| Ex. # | Structure | IUPAC Name | [M + H]⁺ |
| 391 | 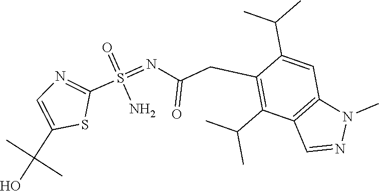 [SMILES: CC(C)c1cc2c(cnn2C)c(C(C)C)c1CC(=O)N=S(N)(=O)c1ncc(C(C)(C)O)s1] | N-(Amino(5-(2-hydroxypropan-2-yl)thiazol-2-yl)(oxo)-λ⁶- sulfaneylidene)-2-(4,6-diisopropyl-1-methyl-1H-indazol-5- yl)acetamide | 478 |
| 392 | 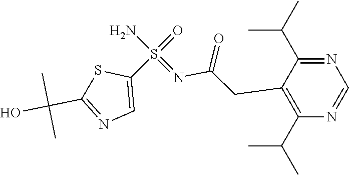 [SMILES: CC(C)c1ncnc(C(C)C)c1CC(=O)N=S(N)(=O)c1cnc(C(C)(C)O)s1] | N-(amino(2-(2-hydroxypropan-2-yl)thiazol-5-yl)(oxo)-λ⁶- sulfaneylidene)-2-(4,6-diisopropylpyrimidin-5-yl)acetamide | 426 |
| 393 | 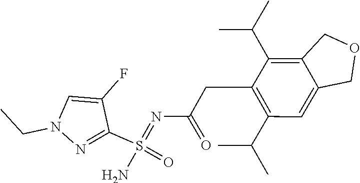 [SMILES: CCn1cc(F)c(S(N)(=O)=NC(=O)Cc2c(C(C)C)cc3c(c2C(C)C)COC3)n1] | N-(amino(1-ethyl-4-fluoro-1H-pyrazol-3-yl)(oxo)-λ⁶- sulfaneylidene)-2-(4,6-diisopropyl-1,3-dihydroisobenzofuran- 5-yl)acetamide | 437 |
| 394 | 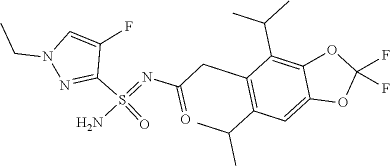 [SMILES: CCn1cc(F)c(S(N)(=O)=NC(=O)Cc2c(C(C)C)cc3c(c2C(C)C)OC(F)(F)O3)n1] | N-(amino(1-ethyl-4-fluoro-1H-pyrazol-3-yl)(oxo)-λ⁶- sulfaneylidene)-2-(2,2-difluoro-4,6- diisopropylbenzo[d][1,3]dioxol-5-yl)acetamide | 475 |
| 395 | 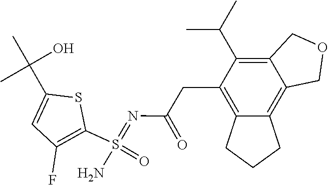 [SMILES: CC(C)c1c2c(c3c(c1CC(=O)N=S(N)(=O)c1sc(C(C)(C)O)cc1F)CCC3)COC2] | N-(Amino(3-fluoro-5-(2-hydroxypropan-2-yl)thiophen-2-yl) (oxo)-λ⁶-sulfaneylidene)-2-(4-isopropyl-3,6,7,8-tetrahydro- 1H-indeno[4,5-c]furan-5-yl)acetamide | 481 |

TABLE 46
Examples in the following table were obtained from chiral HPLC resolutions of
racemic or diastereomeric mixture examples described above. The chiral column and eluents are
listed in the table. As a convention, the faster-eluting enantiomer is always listed first in the table
followed by the slower-eluting enantiomer of the pair. The symbol * at a chiral center denotes
that this chiral center has been resolved and the absolute stereochemistry at that center has not
been determined. Assigned stereochemistry in compound names are tentative.

| Ex. | | | | | LC-MS |
|---|---|---|---|---|---|
| # | Structure | IUPAC Name | Column | Eluents | [M + H]⁺ |
| 396 | 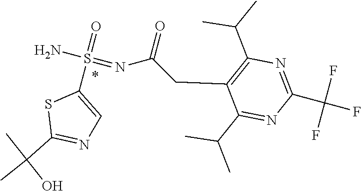 [SMILES: CC(C)c1nc(C(F)(F)F)nc(C(C)C)c1CC(=O)N=S(N)(=O)c1cnc(C(C)(C)O)s1] | (S) or (R)-N-(amino(2-(2- hydroxypropan-2-yl) thiazol-5-yl)(oxo)-λ⁶- sulfaneylidene)-2-(4,6- diisopropyl-2- (trifluoromethyl)pyrimidin- 5-yl)acetamide (Separated from Example 153) | Chiralpak IA, 2 * 25 cm, 5 um | 5% EtOH in Hex (0.1% FA) | 494 |
| 397 | 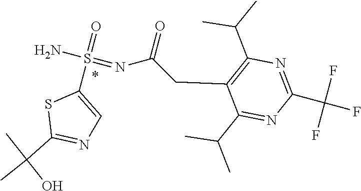 [SMILES: CC(C)c1nc(C(F)(F)F)nc(C(C)C)c1CC(=O)N=S(N)(=O)c1cnc(C(C)(C)O)s1] | (R) or (S)-N-(amino(2-(2- hydroxypropan-2-yl) thiazol-5-yl)(oxo)-λ⁶- sulfaneylidene)-2-(4,6- diisopropyl-2- (trifluoromethyl)pyrimidin- 5-yl)acetamide (Separated from Example 153) | Chiralpak IA, 2 * 25 cm, 5 um | 5% EtOH in Hex (0.1% FA) | 494 |
| 398 | 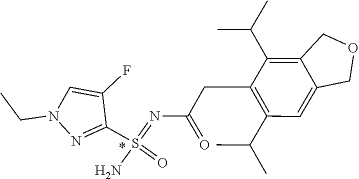 [SMILES: CCn1cc(F)c(S(N)(=O)=NC(=O)Cc2c(C(C)C)cc3c(c2C(C)C)COC3)n1] | (R) or (S)-N-(amino(1- ethyl-4-fluoro-1H- pyrazol-3-yl)(oxo)-λ⁶- sulfaneylidene)-2-(4,6- diisopropyl-1,3- dihydroisobenzofuran-5- yl)acetamide | CHIRALPAK IE, 4.6 * 50 mm 3, um | 15% EtOH in Hex (0.1% FA) | 437 |
| 399 | 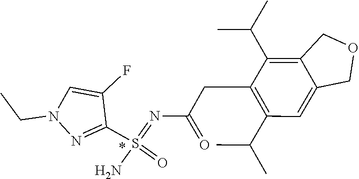 [SMILES: CCn1cc(F)c(S(N)(=O)=NC(=O)Cc2c(C(C)C)cc3c(c2C(C)C)COC3)n1] | (S) or (R)-N-(amino(1- ethyl-4-fluoro-1H- pyrazol-3-yl)(oxo)-λ⁶- sulfaneylidene)-2-(4,6- diisopropyl-1,3- dihydroisobenzofuran-5- yl)acetamide | CHIRALPAK IE, 4.6 * 50 mm, 3 um | 15% EtOH in Hex (0.1% FA) | 437 |
| 400 | 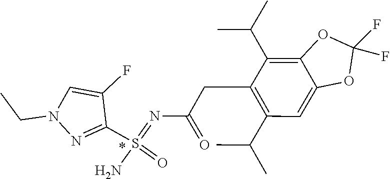 [SMILES: CCn1cc(F)c(S(N)(=O)=NC(=O)Cc2c(C(C)C)cc3c(c2C(C)C)OC(F)(F)O3)n1] | (R) or (S)-N-(amino(1- ethyl-4-fluoro-1H- pyrazol-3-yl)(oxo)-λ⁶- sulfaneylidene)-2-(2,2- difluoro-4,6- diisopropylbenzo[d][1,3] dioxol-5-yl)acetamide | Chiralpak ID, 2 * 25 cm, 5 um | 50% IPA in Hex (0.1% FA) | 475 |
| 401 | 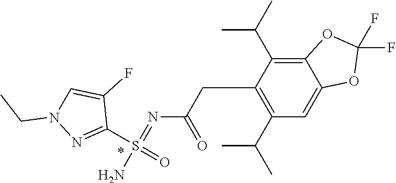 [SMILES: CCn1cc(F)c(S(N)(=O)=NC(=O)Cc2c(C(C)C)cc3c(c2C(C)C)OC(F)(F)O3)n1] | (S) or (R)-N-(amino(1- ethyl-4-fluoro-1H- pyrazol-3-yl)(oxo)-λ⁶- sulfaneylidene)-2-(2,2- difluoro-4,6- diisopropylbenzo[d][1,3] dioxol-5-yl)acetamide | Chiralpak ID, 2 * 25 cm, 5 um | 50% IPA in Hex (0.1% FA) | 475 |
| 402 | 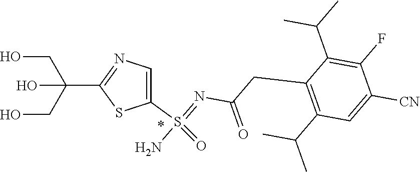 [SMILES: CC(C)c1cc(C#N)c(F)c(C(C)C)c1CC(=O)N=S(N)(=O)c1cnc(C(O)(CO)CO)s1] | (R) or (S)-N-(amino(oxo) (2-(1,2,3- trihydroxypropan-2-yl) thiazol-5-yl)-λ⁶- sulfaneylidene)-2-(4- cyano-3-fluoro-2,6- diisopropylphenyl)acetamide | CHIRALPAK IC, 3 * 25 cm, 5 um | 15% IPA in Hex (0.1% FA) | 499 |
| 403 | 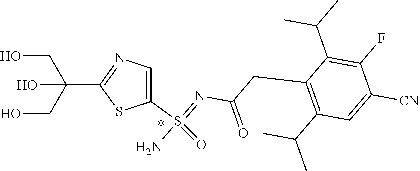 [SMILES: CC(C)c1cc(C#N)c(F)c(C(C)C)c1CC(=O)N=S(N)(=O)c1cnc(C(O)(CO)CO)s1] | (S) or (R)-N-(amino(oxo) (2-(1,2,3- trihydroxypropan-2-yl) thiazol-5-yl)-λ⁶- sulfaneylidene)-2-(4- cyano-3-fluoro-2,6- diisopropylphenyl)acetamide | CHIRALPAK IC, 3 * 25 cm, 5 um | 15% IPA in Hex (0.1% FA) | 499 |

### Example 404 (Compound 1401)

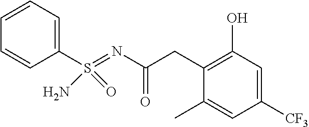 [SMILES: Cc1cc(C(F)(F)F)cc(O)c1CC(=O)N=S(N)(=O)c1ccccc1]

### N-(amino(oxo)(phenyl)-λ⁶-sulfaneylidene)-2-(2-hydroxy-6-methyl-4-(trifluoromethyl)phenyl)acetamide (Scheme XIII)

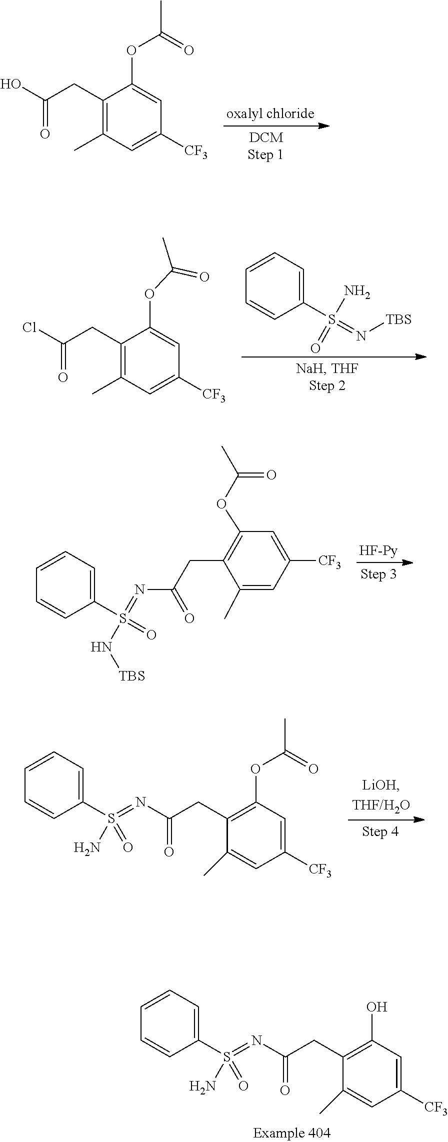

### Step 1: 2-(2-Chloro-2-oxoethyl)-3-methyl-5-(trifluoromethyl)phenyl Acetate

To a stirred solution of 2-(2-acetoxy-6-methyl-4-(trifluoromethyl)phenyl)acetic acid (100 mg, 0.362 mmol) and TEA (110 mg, 1.09 mmol) in DCM (5 mL) under nitrogen was added oxalyl chloride (92 mg, 0.724 mmol) dropwise at 0° C. The resulting solution was stirred for 1 h at RT. The resulting solution was concentrated under vacuum. This resulted in the title compound (crude) as a yellow solid which was used for next step without further purification.

### Step 2: 2-(2-((((Tert-butyldimethylsilyl)amino)(oxo)(phenyl)-λ⁶-sulfaneylidene)amino)-2-oxoethyl)-3-methyl-5-(trifluoromethyl)phenyl Acetate

To a stirred solution of N′-(tert-butyldimethylsilyl)benzenesulfonimidamide (91.8 mg, 0.34 mmol) in THF (5 mL) under nitrogen was added NaH (60% wt., 27 mg, 0.68 mmol) at 0° C., followed by the addition of 2-(2-chloro-2-oxoethyl)-3-methyl-5-(trifluoromethyl)phenyl acetate (crude from last step) in THF (5 mL) dropwise at 0° C. The resulting solution was stirred for 2 h at RT. The reaction was then quenched with 5 mL of water, extracted with 3×20 mL of EtOAc. The combined organic layers was dried over anhydrous sodium sulfate and concentrated under vacuum. This resulted in 100 mg (crude) of the title compound as a yellow solid. MS-ESI: 529 (M+1).

### Step 3: 2-(2-((amino(oxo)(phenyl)-λ⁶-sulfaneylidene)amino)-2-oxoethyl)-3-methyl-5-(trifluoromethyl)-phenyl Acetate

To a stirred solution of 2-(2-((((tert-butyldimethylsilyl)amino)(oxo)(phenyl)-λ⁶-sulfaneylidene)amino)-2-oxoethyl)-3-methyl-5-(trifluoromethyl)phenyl acetate (100 mg, crude from last step) in THF (2 mL) was added HF-Pyridine (70% wt., 38 mg) dropwise at 0° C. The resulting solution was stirred for 1 h at RT. The resulting solution was concentrated under vacuum. This resulted in 70 mg (crude) of the title compound. MS-ESI: 415 (M+1).

### Step 4: N-(amino(oxo)(phenyl)-λ⁶-sulfaneylidene)-2-(2-hydroxy-6-methyl-4-(trifluoromethyl)phenyl)-acetamide

To a stirred solution of 2-(2-((amino(oxo)(phenyl)-λ⁶-sulfaneylidene)amino)-2-oxoethyl)-3-methyl-5-(trifluoromethyl)phenyl acetate (70 mg, crude from last step) in THF (2 mL) and water (2 mL) was added LiOH (8.2 mg, 0.34 mmol) at 0° C. The resulting solution was stirred for 2 h at RT. The pH value was adjusted to 7 with HCl (6 M). The crude mixture was purified by Prep-HPLC using the following conditions: Column, Sunfire prep C18 column, 30*150, Sum; mobile phase, Water and ACN (30% ACN up to 60% over 7 min); Detector, UV 254 nm. This resulted in 6.4 mg (5.1% over three steps) of Example 404 (compound 1401) as colorless oil. MS-ESI: 371 (M−1). ¹H NMR (400 MHz, CD₃OD) δ 7.99-7.92 (m, 2H), 7.68-7.60 (m, 1H), 7.59-7.52 (m, 2H), 6.92 (d, J=1.8 Hz, 1H), 6.88 (d, J=1.9 Hz, 1H), 3.80-3.70 (m, 2H), 2.26 (s, 3H).

### Example 405 (Compound 1402a) and Example 406 (Compound 1402b)

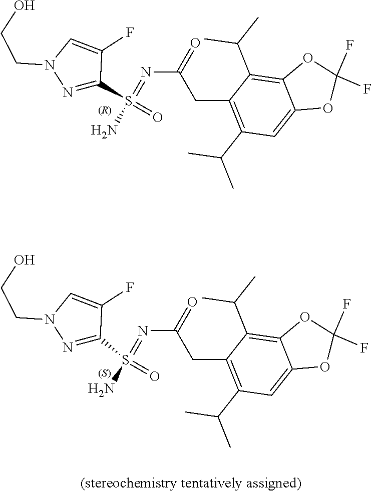

### (R) and (S)—N-(amino(4-fluoro-1-(2-hydroxyethyl)-1H-pyrazol-3-yl)(oxo)-λ⁶-sulfaneylidene)-2-(2,2-difluoro-4,6-diisopropylbenzo[d][1,3]dioxol-5-yl)acetamide (Scheme VI)

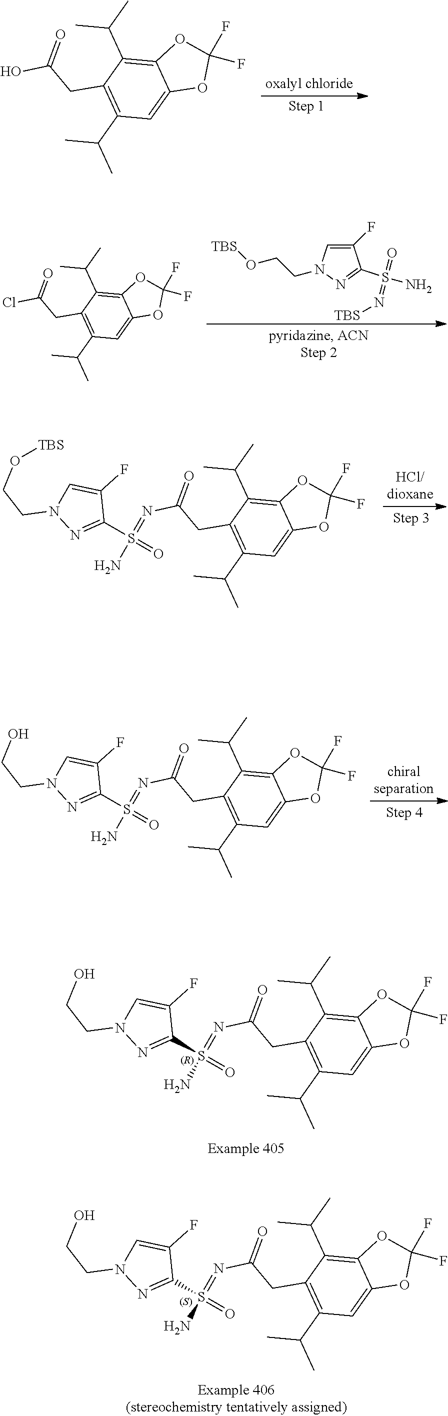

Steps 1-3 used identical procedures as steps 1-3 of Example 348 and 349 from 2-(2,2-difluoro-4,6-diisopropylbenzo[d][1,3]dioxol-5-yl)acetic acid to afford N-(amino(4-fluoro-1-(2-hydroxyethyl)-1H-pyrazol-3-yl)(oxo)-λ⁶-sulfaneylidene)-2-(2,2-difluoro-4,6-diisopropylbenzo[d][1,3]dioxol-5-yl)acetamide. MS-ESI: 491 (M+1).

### Step 4: Chiral Separation

A 55 mg sample of N-(amino(4-fluoro-1-(2-hydroxyethyl)-1H-pyrazol-3-yl)(oxo)-λ⁶-sulfaneylidene)-2-(2,2-difluoro-4,6-diisopropylbenzo[d][1,3]dioxol-5-yl)acetamide was resolved using the follow condition: Column: Chiralpak ID, 2*25 cm, 5 um; Mobile Phase A: Hex (0.1% FA), Mobile Phase B: IPA; Flow rate: 20 mL/min; Gradient: 15% B to 15% B over 13.5 min; 220/254 nm; Rt₁: 5.94 min (Example 405); RT₂: 11.2 min (Example 406); This resulted in 19.6 mg of Example 405 and 16.6 mg of Example 406. MS-ESI: both 491 (M+1).

Example 405 (compound 1402a): ¹H NMR (400 MHz, DMSO-d₆) δ 7.99 (d, J=4.6 Hz, 1H), 7.93 (br s, 2H), 7.13 (s, 1H), 5.01 (t, J=5.2 Hz, 1H), 4.16-4.07 (m, 2H), 3.75-3.65 (m, 4H), 3.15-3.05 (m, 2H), 1.21 (d, J=6.9 Hz, 3H), 1.16 (d, J=6.9 Hz, 3H), 1.11 (d, J=6.7 Hz, 3H), 1.07 (d, J=6.8 Hz, 3H).

Example 406 (compound 1402b): ¹H NMR (400 MHz, DMSO-d₆) δ 7.99 (d, J=4.6 Hz, 1H), 7.92 (br s, 2H), 7.13 (s, 1H), 5.01 (t, J=5.2 Hz, 1H), 4.16-4.07 (m, 2H), 3.75-3.65 (m, 4H), 3.15-3.05 (m, 2H), 1.21 (d, J=6.9 Hz, 3H), 1.16 (d, J=6.9 Hz, 3H), 1.11 (d, J=6.7 Hz, 3H), 1.07 (d, J=6.8 Hz, 3H).

### Example 407 (Compound 1403a) and Example 408 (Compound 1403b)

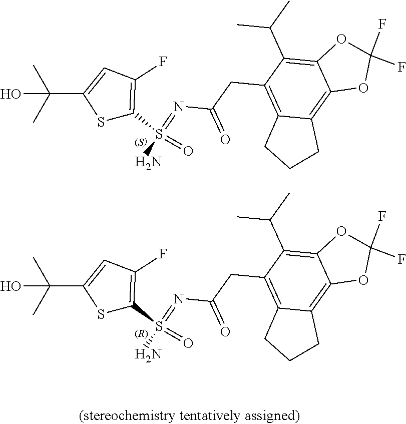

### (R) and (S)—N-(amino(3-fluoro-5-(2-hydroxypropan-2-yl)thiophen-2-yl)(oxo)-λ⁶-sulfaneylidene)-2-(2,2-difluoro-4-isopropyl-7,8-dihydro-6H-indeno[4,5-d][1,3]dioxol-5-yl)acetamide (Scheme VI)

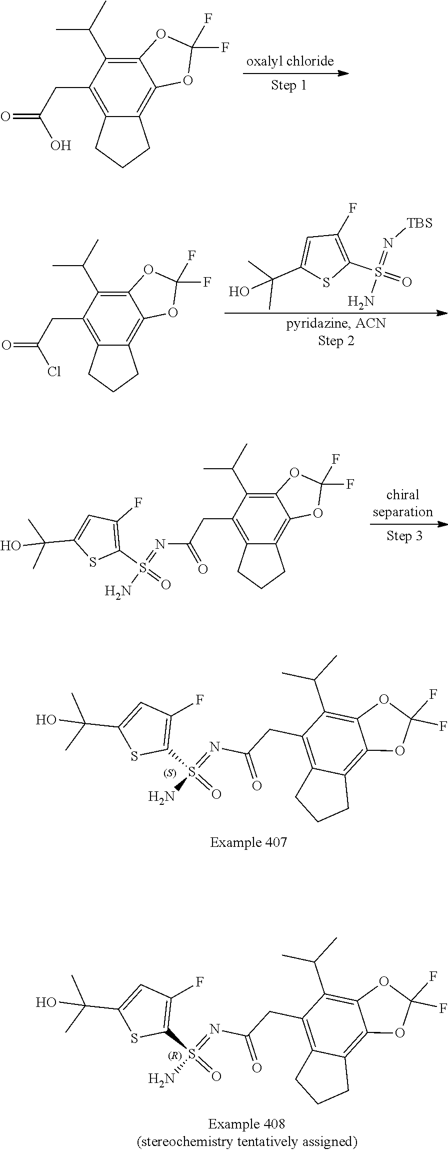

Steps 1-2 used identical procedures as step 1 of Example 4 from 2-(2,2-difluoro-4-isopropyl-7,8-dihydro-6H-indeno[4,5-d][1,3]dioxol-5-yl)acetic acid to afford N-(amino(3-fluoro-5-(2-hydroxypropan-2-yl)thiophen-2-yl)(oxo)-λ⁶-sulfaneylidene)-2-(2,2-difluoro-4-isopropyl-7,8-dihydro-6H-indeno[4,5-d][1,3]dioxol-5-yl)acetamide. MS-ESI: 519 (M+1).

### Step 4: Chiral Separation

A 40 mg sample of N-(amino(4-fluoro-1-(2-hydroxyethyl)-1H-pyrazol-3-yl)(oxo)-λ⁶-sulfaneylidene)-2-(2,2-difluoro-4,6-diisopropylbenzo[d][1,3]dioxol-5-yl)acetamide was resolved by the follow condition: Column: CHIRAL ART Cellulose-SB, 2*25 cm, 5 um; Mobile Phase A: Hex (0.1% FA), Mobile Phase B: EtOH; Flow rate: 20 mL/min; Gradient: 10% B to 10% B over 14 min; 254/220 nm; Rt₁: 11.78 min (Example 407); Rt₂: 13.19 min (Example 408); This resulted in 14.5 mg of Example 407 and 14.3 mg of Example 408. MS-ESI: both 519 (M+1).

Example 407 (compound 1403a): ¹H NMR (400 MHz, DMSO-d₆) δ 8.06 (br s, 2H), 6.98 (s, 1H), 5.88 (s, 1H), 3.65-3.50 (m, 2H), 3.20-3.00 (m, 1H), 2.90 (t, J=7.5 Hz, 2H), 2.82 (t, J=7.6 Hz, 2H), 2.10-1.99 (m, 2H), 1.45 (s, 3H), 1.44 (s, 3H), 1.20 (d, J=7.8 Hz, 3H), 1.17 (d, J=7.8 Hz, 3H).

Example 408 (compound 1403b): ¹H NMR (400 MHz, DMSO-d₆) δ 8.06 (br s, 2H), 6.94 (s, 1H), 5.84 (s, 1H), 3.61-3.46 (m, 2H), 3.16-2.94 (m, 1H), 2.86 (t, J=7.5 Hz, 2H), 2.78 (t, J=7.6 Hz, 2H), 2.10-1.99 (m, 2H), 1.45 (s, 3H), 1.44 (s, 3H), 1.19 (d, J=7.8 Hz, 3H), 1.15 (d, J=7.8 Hz, 3H).

TABLE 47
Examples in the following table were prepared using similar conditions as described in
Example 379 and Scheme VI from appropriate starting material.

| | | | | Exact Mass |
|---|---|---|---|---|
| Ex. # | Compound # | Structure | IUPAC Name | [M + H]⁺ |
| 409 | 1404 | 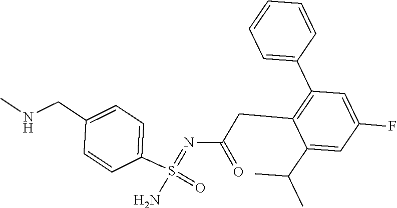 [SMILES: CNCc1ccc(S(N)(=O)=NC(=O)Cc2c(-c3ccccc3)cc(F)cc2C(C)C)cc1] | N-(amino(4- ((methylamino)methyl)phenyl) (oxo)-λ⁶-sulfaneylidene)- 2-(5-fluoro-3-isopropyl- [1,1′-biphenyl]-2-yl) acetamide | 454 |

TABLE 48
Examples in the following table were prepared using similar conditions as described in
Example 4 and Scheme 2 from appropriate starting materials.

| | | | | Exact Mass |
|---|---|---|---|---|
| Ex. # | Compound # | Structure | IUPAC Name | [M + H]⁺ |
| 410 | 1405 | 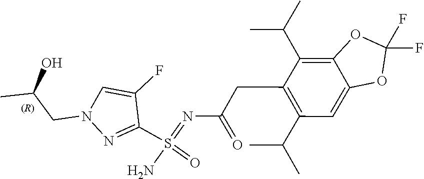 | N-(amino(4-fluoro-1-((R)-2- hydroxypropyl)-1H-pyrazol-3- yl)(oxo)-λ⁶-sulfaneylidene)-2- (2,2-difluoro-4,6- diisopropylbenzo[d][1,3]dioxol- 5-yl)acetamide | 505 |

TABLE 49
Examples in the following table were obtained from chiral HPLC resolutions of
racemic and diastereomeric mixture examples described above. The chiral column and eluents
are listed in the table. As a convention, the faster-eluting enantiomer is always listed first in the
table followed by the slower-eluting enantiomer of the pair. The symbol * at a chiral center
denotes that this chiral center has been resolved and the absolute stereochemistry at that center
has not been determined. Assigned stereochemistry in compound names are tentative.

| | Com- | | | | | |
|---|---|---|---|---|---|---|
| Ex. | pound | | | | | LC-MS |
| # | # | Structure | IUPAC Name | Column | Eluents | [M + H]⁺ |
| 411 | 1406a | 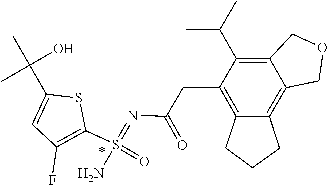 [SMILES: CC(C)c1c2c(c3c(c1CC(=O)N=S(N)(=O)c1sc(C(C)(C)O)cc1F)CCC3)COC2] | (R) or (S)-N-(amino(3- fluoro-5-(2-hydroxypropan- 2-yl)thiophen-2-yl)(oxo)- λ⁶-sulfaneylidene)-2-(4- isopropyl-3,6,7,8- tetrahydro- 1H-indeno[4,5-c]furan-5- yl)acetamide | CHIRAL PAK IG 2 * 25 cm, 5 um | 30% EtOH in Hex (0.1% FA) | 481 |
| 412 | 1406b | 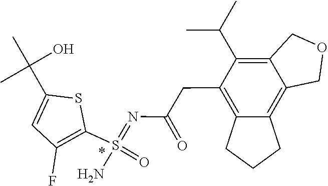 [SMILES: CC(C)c1c2c(c3c(c1CC(=O)N=S(N)(=O)c1sc(C(C)(C)O)cc1F)CCC3)COC2] | (S) or (R)-N-(amino(3- fluoro-5-(2-hydroxypropan- 2-yl)thiophen-2-yl)(oxo)- λ⁶-sulfaneylidene)-2-(4- isopropyl-3,6,7,8- tetrahydro- 1H-indeno[4,5-c]furan-5- yl)acetamide | CHIRAL PAK IG 2 * 25 cm, 5 um | 30% EtOH in Hex (0.1% FA) | 481 |
| 413 | 1407a | 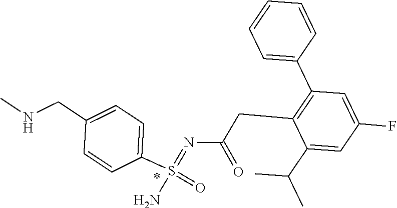 [SMILES: CNCc1ccc(S(N)(=O)=NC(=O)Cc2c(-c3ccccc3)cc(F)cc2C(C)C)cc1] | (S) or (R)-N-(amino(4- ((methylamino)methyl) phenyl) (oxo)-λ⁶-sulfaneylidene)- 2-(5-fluoro-3-isopropyl- [1,1′-biphenyl]-2- yl)acetamide | CHIRAL PAK IH, 2.0 * 25 cm, 5 um | 10% EtOH in Hex (8 mM NH₃ • MeOH) | 454 |
| 414 | 1407b | 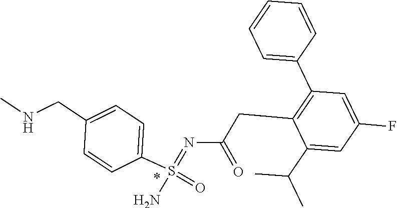 [SMILES: CNCc1ccc(S(N)(=O)=NC(=O)Cc2c(-c3ccccc3)cc(F)cc2C(C)C)cc1] | (R) or (S)-N-(amino(4- ((methylamino)methyl) phenyl) (oxo)-λ⁶-sulfaneylidene)- 2-(5-fluoro-3-isopropyl- [1,1′-biphenyl]-2- yl)acetamide | CHIRAL PAK IH, 2.0 * 25 cm, 5 um | 10% EtOH in Hex (8 mM NH₃ • MeOH) | 454 |
| 415 | 1408a | 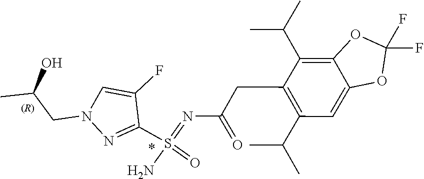 | (R) or (S)-N-(amino(4- fluoro-1-((R)-2- hydroxypropyl)-1H- pyrazol-3-yl)(oxo)-λ⁶- sulfaneylidene)-2-(2,2- difluoro-4,6- diisopropylbenzo[d][1,3] dioxol-5-yl)acetamide | Chiralpak ID, 2 * 25 cm, 5 um | 20% IPA in Hex (0.1% FA) | 505 |
| 416 | 1408b | 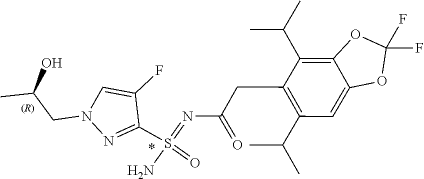 | (S) or (R)-N-(amino(4- fluoro-1-((R)-2- hydroxypropyl)-1H- pyrazol-3-yl)(oxo)-λ⁶- sulfaneylidene)-2-(2,2- difluoro-4,6- diisopropylbenzo[d][1,3] dioxol-5-yl)acetamide | Chiralpak ID, 2 * 25 cm, 5 um | 20% IPA in Hex (0.1% FA) | 505 |

The following protocol is suitable for testing the activity of the compounds disclosed herein.

Procedure 1: IL-1β Production in PMA-Differentiated THP-1 Cells Stimulated with Gramicidin.

THP-1 cells were purchased from the American Type Culture Collection and sub-cultured according to instructions from the supplier. Cells were cultured in complete RPMI 1640 (containing 10% heat inactivated FBS, penicillin (100 units/ml) and streptomycin (100 μg/ml)), and maintained in log phase prior to experimental setup. Prior to the experiment, compounds were dissolved in dimethyl sulfoxide (DMSO) to generate a 30 mM stock. The compound stock was first pre-diluted in DMSO to 3, 0.34, 0.042 and 0.0083 mM intermediate concentrations and subsequently spotted using Echo550 liquid handler into an empty 384-well assay plate to achieve desired final concentration (e.g. 100, 33, 11, 3.7, 1.2, 0.41, 0.14, 0.046, 0.015, 0.0051, 0.0017 μM). DMSO was backfilled in the plate to achieve a final DMSO assay concentration of 0.37%. The plate was then sealed and stored at room temperature until required.

THP-1 cells were treated with PMA (Phorbol 12-myristate 13-acetate) (20 ng/ml) for 16-18 hours. On the day of the experiment the media was removed and adherent cells were detached with trypsin for 5 minutes. Cells were then harvested, washed with complete RPMI 1640, spun down, and resuspended in RPMI 1640 (containing 2% heat inactivated FBS, penicillin (100 units/ml) and streptomycin (100 μg/ml). The cells were plated in the 384-well assay plate containing the spotted compounds at a density of 50,000 cells/well (final assay volume 50 μl). Cells were incubated with compounds for 1 hour and then stimulated with gramicidin (5 μM) (Enzo) for 2 hours. Plates were then centrifuged at 340 g for 5 min. Cell free supernatant (40 μL) was collected using a 96-channel PlateMaster (Gilson) and the production of IL-1β was evaluated by HTRF (cisbio). The plates were incubated for 18 h at 4° C. and read using the preset HTRF program (donor emission at 620 nm, acceptor emission at 668 nm) of the SpectraMax i3x spectrophotometer (Molecular Devices, software SoftMax 6). A vehicle only control and a dose titration of CRID3 (100-0.0017 μM) were run concurrently with each experiment. Data was normalized to vehicle-treated samples (equivalent to 0% inhibition) and CRID3 at 100 μM (equivalent to 100% inhibition). Compounds exhibited a concentration-dependent inhibition of IL-1β production in PMA-differentiated THP-1 cells.

Procedure 2: IL-1β Production in PMA-Differentiated THP-1 Cells Stimulated with Gramicidin.

THP-1 cells were purchased from the American Type Culture Collection and sub-cultured according to instructions from the supplier. Prior to experiments, cells were cultured in complete RPMI 1640 (containing 10% heat inactivated FBS, penicillin (100 units/ml) and streptomycin (100 μg/ml)), and maintained in log phase prior to experimental setup. Prior to the experiment THP-1 were treated with PMA (Phorbol 12-myristate 13-acetate) (20 ng/ml) for 16-18 hours. Compounds were dissolved in dimethyl sulfoxide (DMSO) to generate a 30 mM stock. On the day of the experiment the media was removed and adherent cells were detached with trypsin for 5 minutes. Cells were then harvested, washed with complete RPMI 1640, spun down, resuspended in RPMI 1640 (containing 2% heat inactivated FBS, penicillin (100 units/ml) and streptomycin (100 μg/ml). The cells were plated in a 384-well plate at a density of 50,000 cells/well (final assay volume 50 μl). Compounds were first dissolved in assay medium to obtain a 5× top concentration of 500 μM. 10 step dilutions (1:3) were then undertaken in assay medium containing 1.67% DMSO. 5× compound solutions were added to the culture medium to achieve desired final concentration (e.g. 100, 33, 11, 3.7, 1.2, 0.41, 0.14, 0.046, 0.015, 0.0051, 0.0017 μM). Final DMSO concentration was at 0.37%. Cells were incubated with compounds for 1 hour and then stimulated with gramicidin (5 μM) (Enzo) for 2 hours. Plates were then centrifuged at 340 g for 5 min. Cell free supernatant (40 μL) was collected using a 96-channel PlateMaster (Gilson) and the production of IL-1β was evaluated by HTRF (cisbio). A vehicle only control and a dose titration of CRID3 (100-0.0017 μM) were run concurrently with each experiment. Data was normalized to vehicle-treated samples (equivalent to 0% inhibition) and CRID3 at 100 μM (equivalent to 100% inhibition). Compounds exhibited a concentration-dependent inhibition of IL-1β production in PMA-differentiated THP-1 cells.

### Procedure 3

1. Experimental Procedure

1.1 Cell Culture
1) Culture THP-1 cells in the complete RPMI-1640 medium with 10% FBS at 37° C., 5% CO₂.
2) Passage the cells every 3 days by inoculating 3×10⁵ cells per ml.

1.2 Compound Preparation
Prepare the 3-fold serial dilution of the compounds with DMSO in a 384-well LDV Microplate using TECAN EVO system to generate the compound source plate with 10 concentrations. Top concentration is 30 mM.

1.3 Cell Preparation
1) Centrifuge THP-1 cells at 350 g for 5 min.
2) Re-suspend cells with complete RMPI-1640 medium, and count cells.
3) Seed cells in T225 flask, about 2.5×10⁷ per flask, treat cells with 20 ng/ml PMA (final DMSO concentration<1%).
4) Incubate overnight.

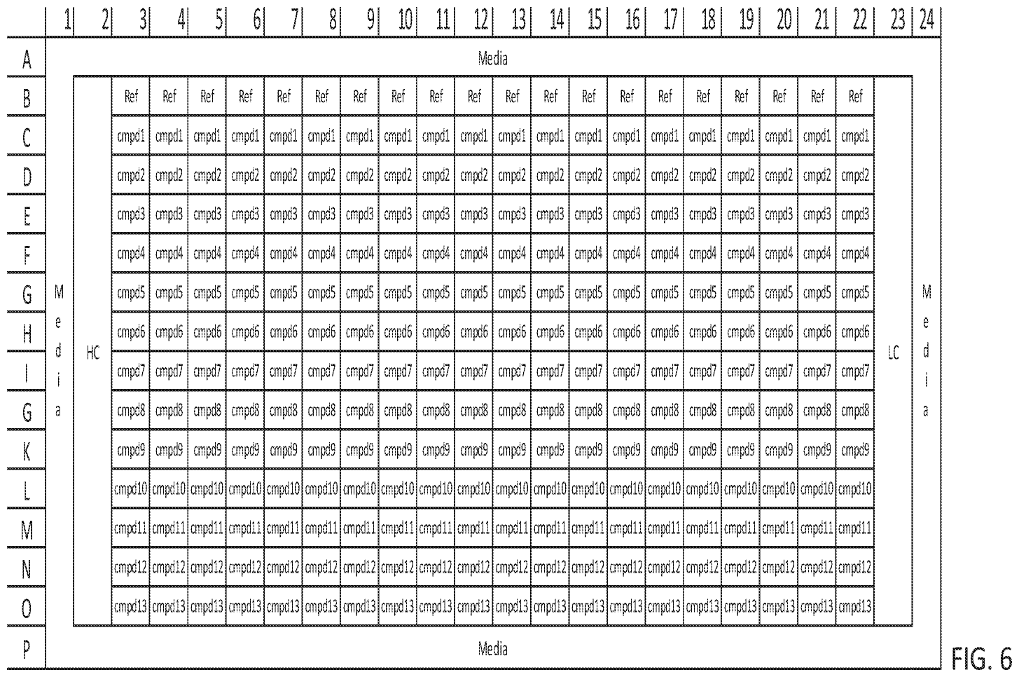

1.4 THP-1 Stimulation
1) Wash adherent THP-1 cells with PBS, and detach cells with 4 ml trypsin for T225 flask.
2) Centrifuge cells at 350 g for 5 min, re-suspend cells with RPMI-1640 containing 2% FBS and count cells with trypan blue.
3) Transfer 50 nl/well the serial dilution of test compound to 384-well plate by Echo; For the high control and first point of CRID3 (MCC950), transfer 165 nl, then backfill to make the DMSO concentration is consistent in all wells, the plate layout is as below.
4) Seed 50 k cells in 40 ul RPMI-1640 with 2% FBS per well in 384-well plate.
5) Incubate for 1 h at 37° C., 5% CO₂.
6) Prepare 5× gramicidin, add 10 μl per well, the final concentration is 5 μM, incubate for 2 hrs at 37° C., 5% CO₂.
7) Centrifuge at 350 g for 1 min.
8) Pipet 16 μl supernatant by apricot, and transfer into white 384 proxiplate. FIG. 6 depicts the layout of the plates: HC: 100 μM CRID3 (MCC950)+5 μM gramicidin LC: 5 μM Gramicidin.

1.5 IL-1β Detection
1) Homogenize the 5× diluent #5 with a vortex and add 1 volume of stock solution in 4 volumes of distilled water.
2) Thaw 20× stock solution of anti-IL1β-Cryptate-antibody and anti-IL1β XL-antibody. Dilute these two antibodies to 1× with detection buffer #3.
3) Pre-mix the two ready-to-use antibody solutions just prior to use.
4) Dispense 4 ul of pre-mixed Anti-IL1β antibodies working solution into all wells.
5) Seal the plate and incubate overnight at 4° C.
6) Read the cell plate using EnVison and plot Readout vs. the test compound concentration to calculate the IC₅₀.

2. Data Analysis:
1. IC₅₀ of compounds can be calculated using the following formulas

Formula for IC₅₀

% inhibition=100−100×[HCₐᵥₑ−Readout/(HCₐᵥₑ−LCₐᵥₑ)]
2. Fit the normalized data in a dose-response manner using XLfit, and calculate the compound concentration.

Tables B1 and B2 show the biological activity of compounds in hTHP-1 assay containing 2% fetal bovine serum; Table B3 shows the biological activity of compounds in hTHP-1 assay containing 2% and 10% fetal bovine serum. Activity key: <0.008 μM=“++++++”; ≥0.008 and <0.04 μM=“+++++”; ≥0.04 and <0.2 μM=“++++”; ≥0.2 and <1 μM=“+++”; ≥1 and <5 μM=“++”; ≥5 and <30 μM=“+”. ND=not determined.

TABLE B1
Average IC₅₀ of compounds in hTHP-1 assay

| Example # | Compound # | hTHP-1 IC₅₀ |
|---|---|---|
| 1 | 131 | ++ |
| 2 | 131b or 131a | ++ |
| 3 | 131a or 131b | >30 μM |
| 4 | 129 | + |
| 5 | 129b or 129a | >30 μM |
| 6 | 129a or 129b | ++ |
| 7 | 132 | +++ |
| 8 | 132b | ++ |
| 9 | 132a | +++ |
| 10 | 134 | + |
| 11 | 137 | + |
| 12 | 136 | >30 μM |
| 13 | 133 | +++ |
| 14 | 141 | +++++ |
| 15 | 139 | ++ |
| 16 | 138 | +++ |
| 17 | 140 | +++++ |
| 18 | 142 | ++++ |
| 19 | 143 | +++++ |
| 20 | 197 | +++ |
| 21 | 101 | ++++ |
| 22 | 152 | +++ |
| 23 | 150 | ++ |
| 24 | 148 | +++ |
| 25 | 147 | ++ |
| 26 | 113 | ++ |
| 27 | 151 | ++ |
| 28 | 114 | ++ |
| 29 | 112 | + |
| 30 | 116 | + |
| 31 | 105 | ++ |
| 32 | 117 | + |
| 33 | 109 | +++ |
| 34 | 146 | ++ |
| 35 | 110 | +++ |
| 36 | 126 | +++ |
| 37 | 104 | ++++ |
| 38 | 130 | +++ |
| 39 | 107 | +++ |
| 40 | 106 | +++ |
| 41 | 135 | + |
| 42 | 133b or 133a | ++++ |
| 43 | 133a or 133b | ++ |
| 44 | 141b or 141a | ++++ |
| 45 | 141a or 141b | ++ |
| 46 | 139b or 139a | >30 μM |
| 47 | 139a or 139b | +++ |
| 48 | 138b or 138a | ++++ |
| 49 | 138a or 138b | + |
| 50 | 140b or 140a | +++++ |
| 51 | 140a or 140b | +++ |
| 52 | 144b or 144a | +++++ |
| 53 | 144a or 144b | ++ |
| 54 | 145b or 145a | +++++ |
| 55 | 145a or 145b | ++ |
| 56 | 197b or 197a | +++ |
| 57 | 197a or 197b | ++ |
| 58 | 116b or 116a | >30 μM |
| 59 | 116a or 116b | + |
| 60 | 106b or 106a | ++++ |
| 61 | 106a or 106b | + |
| 62 | 117b or 117a | ++ |
| 63 | 117a or 117b | >30 μM |
| 64 | 152b or 152a | +++ |
| 65 | 152a or 152b | + |
| 66 | 150b or 150a | +++ |
| 67 | 150a or 150b | >30 μM |
| 68 | 148b or 148a | +++ |
| 69 | 148a or 148b | + |
| 70 | 147b or 147a | +++ |
| 71 | 147a or 147b | + |
| 72 | 114b or 114a | ++ |
| 73 | 114a or 114b | >30 μM |
| 74 | 112b or 112a | >30 μM |
| 75 | 112a or 112b | ++ |
| 76 | 101b or 101a | ++++ |
| 77 | 101a or 101b | ++ |
| 78 | 126b or 126a | +++ |
| 79 | 126a or 126b | + |
| 80 | 104b or 104a | +++++ |
| 81 | 104a or 104b | +++ |
| 82 | 130b or 130a | + |
| 83 | 130a or 130b | ++++ |
| 84 | 107b or 107a | ++++ |
| 85 | 107a or 107b | + |
| 86 | 110b or 110a | ++ |
| 87 | 110a or 110b | ++++ |
| 88 | 241 | +++ |
| 89 | 219 | +++ |
| 90 | 218 | + |
| 91 | 217 | + |
| 92 | 214 | >30 μM |
| 93 | 235 | >30 μM |
| 94 | 230 | ++ |
| 95 | 245 | +++ |
| 96 | 244 | >30 μM |
| 97 | 221 | +++ |
| 98 | 220 | + |
| 99 | 304 | ++ |
| 100 | 301 | +++ |
| 101 | 212 | + |
| 102 | 240 | + |
| 103 | 258 | +++ |
| 104 | 257 | +++ |
| 105 | 256 | ++++ |
| 106 | 201 | ++++ |
| 107 | 255 | + |
| 108 | 254 | ++++ |
| 109 | 253 | ++++ |
| 110 | 252 | +++ |
| 111 | 251 | ++++ |
| 112 | 250 | +++ |
| 113 | 249 | +++++ |
| 114 | 248 | ++ |
| 115 | 247 | +++ |
| 116 | 246 | + |
| 117 | 309 | ++++ |
| 118 | 310 | +++ |
| 119 | 243 | +++ |
| 120 | 242 | +++ |
| 121 | 239 | +++ |
| 122 | 238 | +++ |
| 123 | 237 | +++ |
| 124 | 236 | +++ |
| 125 | 234 | +++ |
| 126 | 233 | +++ |
| 127 | 232 | + |
| 128 | 120 | +++ |
| 129 | 229 | ++++ |
| 130 | 228 | ++ |
| 131 | 308 | + |
| 132 | 227 | ++ |
| 133 | 226 | +++ |
| 134 | 225 | ++ |
| 135 | 224 | ++ |
| 136 | 191 | +++ |
| 137 | 222 | +++ |
| 138 | 307 | ++ |
| 139 | 231 | +++ |
| 140 | 306 | +++ |
| 141 | 305 | ++++ |
| 142 | 216 | ++ |
| 143 | 215 | ++ |
| 144 | 203 | + |
| 145 | 213 | +++ |
| 146 | 204 | + |
| 147 | 202 | ++ |
| 148 | 211 | + |
| 149 | 210 | +++ |
| 150 | 209 | + |
| 151 | 208 | +++ |
| 152 | 207 | +++ |
| 153 | 206 | ++ |
| 154 | 205 | + |
| 155 | 261b | +++ |
| 156 | 261a | +++ |
| 157 | 256a | +++++ |
| 158 | 256b | ++ |
| 159 | 201a | ++++ |
| 160 | 201b | +++ |
| 161 | 258a | ++++ |
| 162 | 258b | + |
| 163 | 249a | ++++ |
| 164 | 249b | ++ |
| 165 | 309b | ++ |
| 166 | 309a | ++++ |
| 167 | 239a | ++ |
| 168 | 239b | +++ |
| 169 | 238a | ++ |
| 170 | 238b | +++ |
| 171 | 221a | +++ |
| 172 | 221b | + |
| 173 | 229a | +++ |
| 174 | 229b | + |
| 175 | 234b | ++ |
| 176 | 234a | +++ |
| 177 | 236a | ++++ |
| 178 | 236b | ++ |
| 179 | 242a | +++ |
| 180 | 242b | + |
| 181 | 243a | ++++ |
| 182 | 243b | + |
| 183 | 227a | +++ |
| 184 | 227b | + |
| 185 | 251b | ++++ |
| 186 | 251a | + |
| 187 | 228a | + |
| 188 | 228b | +++ |
| 189 | 226a | ++ |
| 190 | 226b | ++++ |
| 191 | 225a | ++ |
| 192 | 225b | >30 μM |
| 193 | 244b | >30 μM |
| 194 | 244ab | + |
| 195 | 244aa | >30 μM |
| 196 | 220a | + |
| 197 | 220b | >30 μM |
| 198 | 218a | + |
| 199 | 218b | >30 μM |
| 200 | 217a | ++ |
| 201 | 217b | >30 μM |
| 202 | 303 | +++ |
| 203 | 302 | + |
| 204 | 203b | ++ |
| 205 | 203a | >30 μM |
| 206 | 202a | +++ |
| 207 | 202b | >30 μM |
| 208 | 213a | ++++ |
| 209 | 213b | ++ |
| 210 | 210a | + |
| 211 | 210b | +++ |
| 212 | 216a | >30 μM |
| 213 | 216b | ++ |
| 214 | 110a′ | ++++ |
| 215 | 110b′ | ++ |
| 216 | 263a | +++ |
| 217 | 263b | + |
| 218 | 264a | + |
| 219 | 264b | >30 μM |
| 220 | 263 | ++++ |
| 221 | 264 | + |
| 222 | 1304 | ++ |
| 223 | 1304a | ++ |
| 224 | 1304b | >30 |
| 225 | 1333 | + |
| 226 | 1308 | ++ |
| 227 | 1318 | ++ |
| 228 | 1307 | + |
| 229 | 1319 | ++ |
| 230 | 1332 | ++ |
| 231 | 1336a | +++ |
| 232 | 1302b | + |
| 233 | 1342a | + |
| 234 | 1337a | + |
| 235 | 1337b | >30 |
| 236 | 1330 | ++ |
| 237 | 1334 | + |
| 238 | 1315 | ++ |
| 239 | 1324 | ++ |
| 240 | 1314 | + |
| 241 | 1313 | + |
| 242 | 1335 | >30 |
| 243 | 1328 | + |
| 244 | 1321 | ++ |
| 245 | 1320 | ++ |
| 246 | 265 | ++++ |
| 247 | 1303 | ++ |
| 248 | 1327 | ++ |
| 249 | 1310 | ++ |
| 250 | 1301 | + |
| 251 | 1305 | +++ |
| 252 | 1312 | ++ |
| 253 | 1309 | ++ |
| 254 | 1326 | + |
| 255 | 1325 | + |
| 256 | 1331 | + |
| 257 | 1338 | ++ |
| 258 | 1329 | ++ |
| 259 | 1323 | + |
| 260 | 1322 | + |
| 262 | 1302 | + |
| 263 | 1317 | ++ |
| 264 | 1316 | +++ |
| 265 | 1311 | + |
| 266 | 1306 | ++ |
| 267 | 501 | + |
| 268 | 504 | ++ |
| 269 | 509b | >30 |
| 270 | 509a | + |
| 271 | 222a | +++ |
| 272 | 222b | + |
| 273 | 1330b | >30 |
| 274 | 1330a | ++ |
| 275 | 1324a | +++ |
| 276 | 1324b | + |
| 277 | 1319a | +++ |
| 278 | 1319b | >30 |
| 279 | 1308a | ++ |
| 280 | 1308b | >30 |
| 281 | 1307a | + |
| 282 | 1307b | >30 |
| 283 | 1313a | +++ |
| 284 | 1313b | >30 |
| 285 | 1318a | ++ |
| 286 | 1318b | >30 |
| 287 | 305a | ++++ |
| 288 | 305b | >30 |
| 289 | 1323a | + |
| 290 | 1323b | >30 |
| 291 | 1322a | + |
| 292 | 1322b | >30 |
| 293 | 1321a | +++ |
| 294 | 1321b | + |
| 295 | 1320a | +++ |
| 296 | 1320b | >30 |
| 297 | 1317a | ++ |
| 298 | 1317b | + |
| 299 | 1316b | + |
| 300 | 1316a | +++ |
| 301 | 1311a | ++ |
| 302 | 1311b | >30 |
| 303 | 1306a | ++ |
| 304 | 1306b | >30 |
| 305 | 1327b | >30 |
| 306 | 1327a | +++ |
| 307 | 1310a | +++ |
| 308 | 1310b | >30 |
| 309 | 501b | >30 |
| 310 | 501a | ++ |
| 311 | 504b | + |
| 312 | 504a | +++ |
| 313 | 1335a | + |
| 314 | 1335b | >30 |
| 315 | 205a | + |
| 316 | 205b | >30 |
| 317 | 1331a | ++ |
| 318 | 1331b | >30 |
| 319 | 1328b | + |
| 320 | 1328a | >30 |
| 321 | 265a | ++++ |
| 322 | 265b | + |
| 323 | 1326a | >30 |
| 324 | 1326b | + |
| 325 | 1338a | +++ |
| 326 | 1338b | + |
| 327 | 1332a | >30 |
| 328 | 1332b | ++ |
| 329 | 1325b | >30 |
| 330 | 1325a | ++ |
| 331 | 1329a | ++ |
| 332 | 1329b | >30 |
| 333 | 1315f | ++++ |
| 334 | 1315e | + |
| 335 | 1315d | >30 |
| 336 | 1315b | +++ |
| 337 | 1315a | +++++ |
| 338 | 1312b | +++ |
| 339 | 1312a | >30 |
| 340 | 1343a | >30 |
| 341 | 1344a | +++ |
| 342 | 1344b | ++ |
| 343 | 1345a | + |
| 344 | 1345b | +++ |
| 345 | 1346a | + |
| 346 | 1346b | +++ |
| | 260 | >30 |
| | 259 | >17 μM |
| | 259a | + |
| | 259b | >30 μM |
| | 262a | >30 μM |
| | 262b | + |
| | 1339 | >30 |
| | 1340 | >30 |
| | 1341 | >30 |

TABLE B2
Average IC₅₀ of compounds in hTHP-1 assay

| Example # | hTHP-1 IC₅₀ |
|---|---|
| 347 | ++ |
| 348 | +++ |
| 349 | + |
| 350 | +++ |
| 351 | +++ |
| 352 | +++ |
| 353 | + |
| 354 | >30 |
| 355 | >30 |
| 356 | +++ |
| 357 | + |
| 358 | ++++ |
| 359 | ++ |
| 360 | + |
| 361 | ++ |
| 362 | ++ |
| 363 | +++ |
| 364 | +++ |
| 365 | >30 |
| 366 | + |
| 367 | ++ |
| 368 | ++++ |
| 369 | + |
| 370 | >30 |
| 371 | + |
| 372 | + |
| 373 | + |
| 374 | ++++ |
| 375 | + |
| 376 | +++ |
| 377 | >30 |
| 378 | + |
| 379 | + |
| 380 | >30 |
| 381 | + |
| 382 | ++ |
| 383 | +++ |
| 384 | + |
| 385 | +++ |
| 386 | + |
| 387 | + |
| 388 | >30 |
| 389 | >30 |
| 390 | >30 |
| 391 | >30 |
| 392 | >30 |
| 393 | ++ |
| 394 | +++ |
| 395 | +++ |
| 396 | ++ |
| 397 | >30 |
| 398 | +++ |
| 399 | >30 |
| 400 | +++ |
| 401 | + |
| 402 | +++ |
| 403 | + |

TABLE B3
Average IC₅₀ of compounds in hTHP-1 assay
containing 2% or 10% fetal bovine serum

| | hTHP-1 IC₅₀ | hTHP-1 IC₅₀ |
|---|---|---|
| Example # | in 2% FBS | in 10% FBS |
| 404 | + | ND |
| 405 | +++ | ++++ |
| 406 | + | ++ |
| 407 | + | + |
| 408 | +++ | +++ |
| 409 | ++ | +++ |
| 410 | ND | |
| 411 | +++ | +++ |
| 412 | >30 | >30 |
| 413 | ND | + |
| 414 | ND | +++ |
| 415 | ND | ++++ |
| 416 | ND | + |

### Study Example 1

The CARD8 gene is located within the inflammatory bowel disease (IBD) 6 linkage region on chromosome 19. CARD8 interacts with NLRP3, and Apoptosis-associated Speck-like protein to form a caspase-1 activating complex termed the NLRP3 inflammasome. The NLRP3 inflammasome mediates the production and secretion of interleukin-1β, by processing pro-IL-1β into mature secreted IL-1β. In addition to its role in the inflammasome, CARD8 is also a potent inhibitor of nuclear factor NF-κB. NF-κB activation is essential for the production of pro-IL-1β. Since over-production of IL-1β and dysregulation of NF-κB are hallmarks of Crohn's disease, CARD8 is herein considered to be a risk gene for inflammatory bowel disease. A significant association of CARD8 with Crohn's disease was detected in two British studies with a risk effect for the minor allele of the non-synonymous single-nucleotide polymorphism (SNP) of a C allele at rs2043211. This SNP introduces a premature stop codon, resulting in the expression of a severely truncated protein. This variant CARD8 protein is unable to suppress NF-κB activity, leading to constitutive production of pro-IL-1β, which is a substrate for the NLRP3 inflammasome. It is believed that a gain-of-function mutation in an NLRP3 gene (e.g., any of the gain-of-function mutations described herein, e.g., any of the gain-of-function mutations in an NLRP3 gene described herein) combined with a loss-of-function mutation in a CARD8 gene (e.g., a C allele at rs2043211) results in the development of diseases related to increased NLRP3 inflammasome expression and/or activity. Patients having, e.g., a gain-of-function mutation in an NLRP3 gene and/or a loss-of-function mutation in a CARD8 gene are predicted to show improved therapeutic response to treatment with an NLRP3 antagonist.

A study is designed to determine: whether NLRP3 antagonists inhibit inflammasome function and inflammatory activity in cells and biopsy specimens from patients with Crohn's disease or ulcerative colitis; and whether the specific genetic variants identify patients with Crohn's disease or ulcerative colitis who are most likely to respond to treatment with an NLRP3 antagonist.

The secondary objectives of this study are to: determine if an NLRP3 antagonist reduces inflammasome activity in Crohn's disease and ulcerative biopsy samples (comparing Crohn's disease and ulcerative colitis results with control patient results); determine if an NLRP3 antagonist reduced inflammatory cytokine RNA and protein expression in Crohn's disease and ulcerative colitis samples; determine if baseline (no ex vivo treatment) RNA levels of NLRP3, ASC, and IL-1β are greater in biopsy samples from patients with anti-TNFα agent resistance status; and stratify the results according to presence of specific genetic mutations in genes encoding ATG16L1, NLRP3, and CARD8 (e.g., any of the mutations in the ATG16L1 gene, NLRP3 gene, and CARD8 gene described herein).

### Methods

Evaluation of baseline expression of NLRP3 RNA and quantify inhibition of inflammasome activity by an NLRP3 antagonist in biopsies of disease tissue from patients with Crohn's disease and ulcerative colitis.
Determine if NLRP3 antagonist treatment reduces the inflammatory response in biopsies of disease from patients with Crohn's disease based on decreased expression of inflammatory gene RNA measured with Nanostring.
Sequence patient DNA to detect specific genetic mutations in the ATG16L1 gene, NLRP3 gene, and CARD8 gene (e.g., any of the exemplary mutations in these genes described herein) and then stratify the results of functional assays according to the presence of these genetic mutations.

### Experimental Design

Human subjects and tissue:
   Endoscopic or surgical biopsies from areas of disease in patients with Crohn's disease and ulcerative colitis who are either anti-TNFα treatment naïve or resistant to anti-TNFα treatment; additionally biopsies from control patients (surveillance colonoscopy or inflammation-free areas from patients with colorectal cancer) are studied.
Ex vivo Treatment Model:
   Organ or LPMC culture as determined appropriate
Endpoints to be measured:
   Before ex vivo treatment—NLRP3 RNA level
   After ex vivo treatment—inflammasome activity (either processed IL-1β, processed caspase-1, or secreted IL-1β); RNA for inflammatory cytokines (Nanostring); viable T cell number and/or T cell apoptosis.
Data Analysis Plan:
   Determine if NLRP3 antagonist treatment decreases processed IL-1β, processed caspase-1 or secreted IL-1β, and inflammatory cytokine RNA levels.
   Stratify response data according to treatment status at biopsy and the presence of genetic mutations in the NLRP3 gene, CARD8 gene, and ATG16L1 gene (e.g., any of the exemplary genetic mutations of these genes described herein).

### Study Example 2. Treatment of Anti-TNFα Resistant Patients with NLRP3 Antagonists

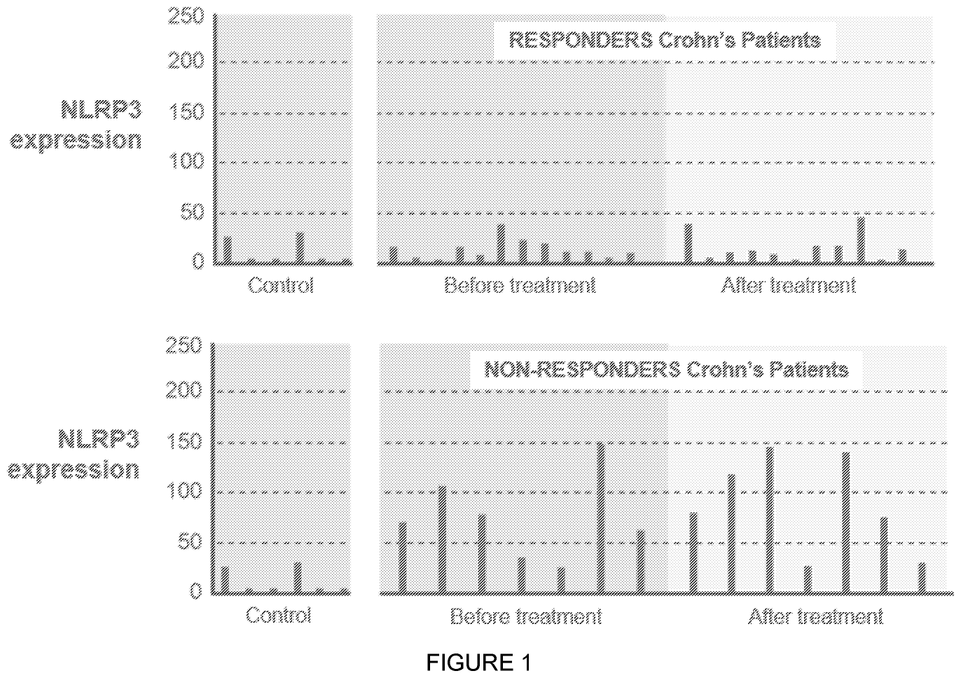

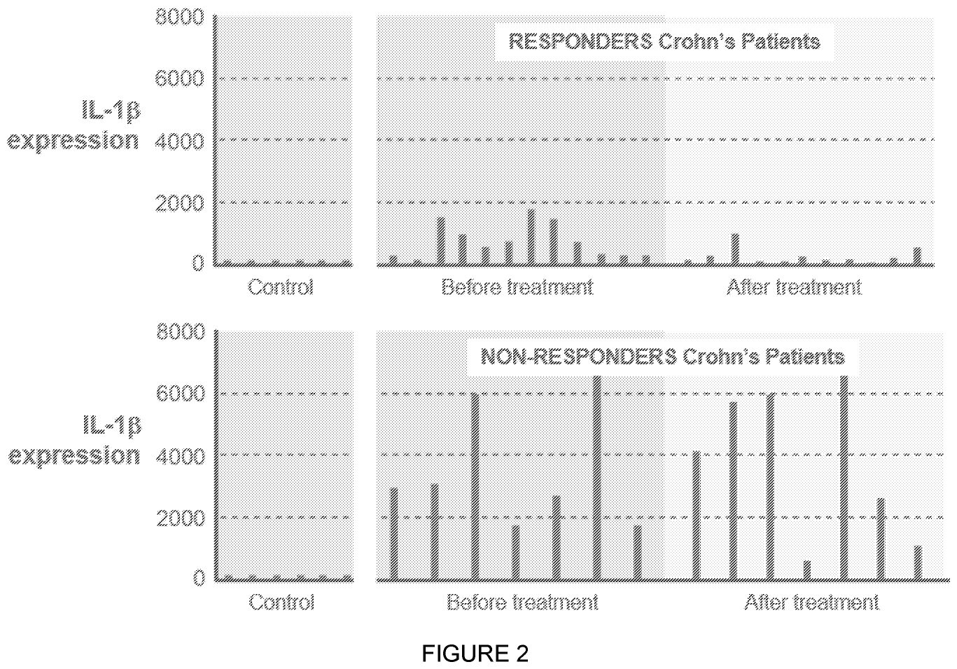

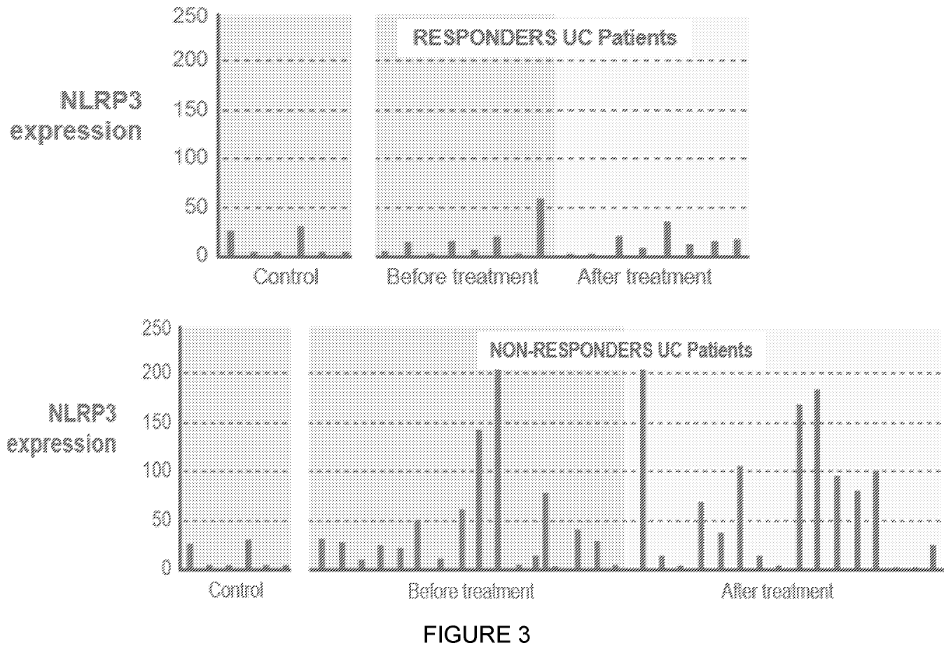

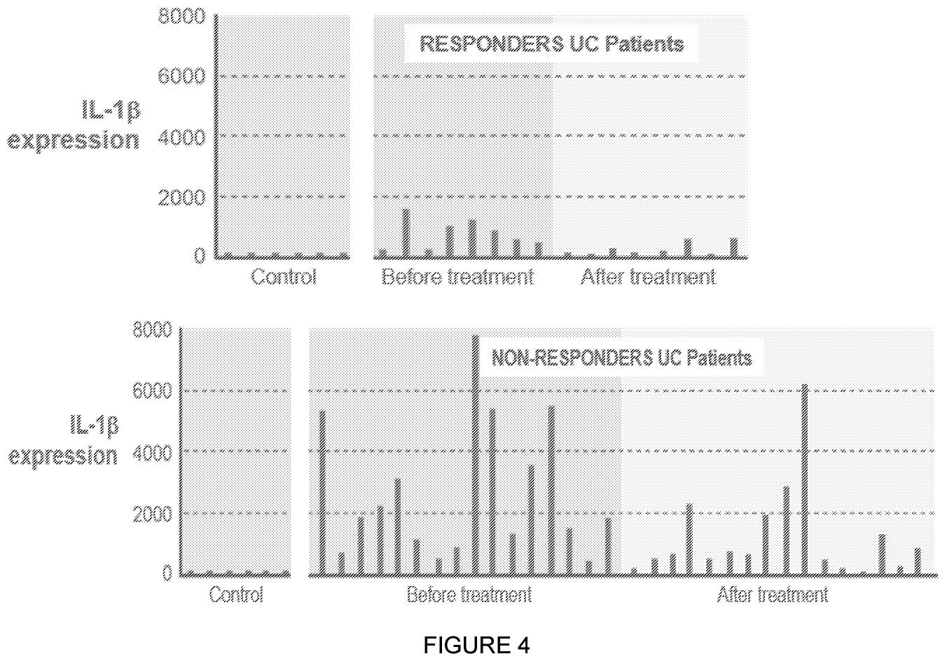

PLoS One 2009 Nov. 24; 4(11):e7984, describes that mucosal biopsies were obtained at endoscopy in actively inflamed mucosa from patients with Ulcerative Colitis, refractory to corticosteroids and/or immunosuppression, before and 4-6 weeks after their first infliximab (an anti-TNFα agent) infusion and in normal mucosa from control patients. The patients in this study were classified for response to infliximab based on endoscopic and histologic findings at 4-6 weeks after first infliximab treatment as responder or non-responder. Transcriptomic RNA expression levels of these biopsies were accessed by the inventors of the invention disclosed herein from GSE 16879, the publically available Gene Expression Omnibus (https://www.ncbi.nlm.nih.gov/geo/geo2r/?acc=GSE16879). Expression levels of RNA encoding NLRP3 and IL-1β were determined using GEO2R (a tool available on the same website), based on probe sets 207075_at and 205067_at, respectively. It was surprisingly found that in Crohn's disease patients that are non-responsive to the infliximab (an anti-TNFα agent) have higher expression of NLRP3 and IL-1β RNA than responsive patients (FIGS. 1 and 2). Similar surprising results of higher expression of NLRP3 and IL-1β RNA in UC patients that are non-responsive to infliximab (an anti-TNFα agent) compared to infliximab (an anti-TNFα agent) responsive patients (FIGS. 3 and 4) were found.

Said higher levels of NLRP3 and IL-1β RNA expression levels in anti-TNFα agent non-responders, is hypothesised herein to lead to NLRP3 activation which in turns leads to release of IL-1β that induces IL-23 production, leading to said resistance to anti-TNFα agents. Therefore, treatment of Crohn's and UC anti-TNFα non-responders with an NLRP3 antagonist would prevent this cascade, and thus prevent development of non-responsiveness to anti-TNFα agents. Indeed, resistance to anti-TNFα agents is common in other inflammatory or autoimmune diseases. Therefore, use of an NLRP3 antagonist for the treatment of inflammatory or autoimmune diseases will block the mechanism leading to non-responsiveness to anti-TNFα□agents. Consequently, use of NLRP3 antagonists will increase the sensitivity of patients with inflammatory or autoimmune diseases to anti-TNFα agents, resulting in a reduced dose of anti-TNFα agents for the treatment of these diseases. Therefore, a combination of an NLRP3 antagonist and an anti-TNFα agent can be used in the treatment of diseases wherein TNFα is overexpressed, such as inflammatory or autoimmune diseases, to avoid such non-responsive development of patients to anti-TNFα agents. Preferably, this combination treatment can be used in the treatment of IBD, for example Crohn's disease and UC.

Further, use of NLRP3 antagonists offers an alternative to anti-TNFα agents for the treatment of diseases wherein TNFα is overexpressed. Therefore, NLRP3 antagonists offers an alternative to anti-TNFα agents inflammatory or autoimmune diseases, such as IBD (e.g. Crohn's disease and UC).

Systemtic anti-TNFα agents are also known to increase the risk of infection. Gut restricted NLRP3 antagonists, however, offers a gut targeted treatment (i.e. non-systemic treatment), preventing such infections. Therefore, treatment of TNFα gut diseases, such as IBD (i.e. Crohn's disease and UC), with gut restricted NLRP3 antagonists has the additional advantage of reducing the risk of infection compared to anti-TNFα agents.

Proposed Experiment:

Determine the expression of NLRP3 and caspase-1 in LPMCs and epithelial cells in patients with non-active disease, in patients with active disease, in patients with active disease resistant to corticosteroids, patients with active disease resistant to TNF-blocking agents. The expression of NLRP3 and caspase-1 in LPMCs and epithelial cells will be analyzed by RNAScope technology. The expression of active NLRP3 signature genes will be analyzed by Nanostring technology. A pilot analysis to determine feasibility will be performed with 5 samples from control, 5 samples from active CD lesions, and 5 samples from active UC lesions.

### Study Example 3

It is presented that NLRP3 antagonists reverse resistance to anti-TNF induced T cell depletion/apoptosis in biopsy samples from IBD patients whose disease is clinically considered resistant or unresponsive to anti-TNF therapy.

A study is designed to determine: whether NLRP3 antagonists inhibit inflammasome function and inflammatory activity in cells and biopsy specimens from patients with Crohn's disease or ulcerative colitis; and whether an NLRP3 antagonist will synergize with anti-TNFα therapy in patients with Crohn's disease or ulcerative colitis.

The secondary objectives of this study are to: determine if an NLRP3 antagonist reduces inflammasome activity in Crohn's disease and ulcerative biopsy samples (comparing Crohn's disease and ulcerative colitis results with control patient results); determine if an NLRP3 antagonist reduced inflammatory cytokine RNA and protein expression in Crohn's disease and ulcerative colitis samples; determine if an NLRP3 antagonist in the absence of co-treatment with anti-TNFα antibody induces T cell depletion in Crohn's disease and ulcerative colitis biopsy samples; and determine if baseline (no ex vivo treatment) RNA levels of NLRP3, ASC, and IL-1β are greater in biopsy samples from patients with anti-TNFα agent resistance status.

### Methods

Evaluation of baseline expression of NLRP3 RNA and quantify inhibition of inflammasome activity by an NLRP3 antagonist in biopsies of disease tissue from patients with Crohn's disease and ulcerative colitis.
Determine if there is synergy between an NLRP3 antagonist and anti-TNF antibody with respect to effects on T cell depletion/apoptosis in biopsies of disease from patients with Crohn's disease and ulcerative colitis.
Determine if NLRP3 antagonist treatment reduces the inflammatory response in biopsies of disease from patients with Crohn's disease based on decreased expression of inflammatory gene RNA measured with Nanostring.

### Experimental Design

Human subjects and tissue:
   Endoscopic or surgical biopsies from areas of disease in patients with Crohn's disease and ulcerative colitis who are either anti-TNFα treatment naïve or resistant to anti-TNFα treatment; additionally biopsies from control patients (surveillance colonoscopy or inflammation-free areas from patients with colorectal cancer) are studied.
Ex vivo Treatment Model:
   Organ or LPMC culture as determined appropriate
Ex vivo Treatments:
   NLRP3 antagonist (2 concentrations), negative control (vehicle), positive control (caspase-1 inhibitor) each in the presence or absence of anti-TNF antibody at a concentration appropriate to distinguish differences in the T cell apoptotic between biopsies from anti-TNF resistant and anti-TNF-sensitive Crohn's disease patients. Each treatment condition is evaluated in a minimum in duplicate samples.
Endpoints to be measured:
   Before ex vivo treatment—NLRP3 RNA level
   After ex vivo treatment—inflammasome activity (either processed IL-1β, processed caspase-1, or secreted IL-1β); RNA for inflammatory cytokines (Nanostring); viable T cell number and/or T cell apoptosis.
Data Analysis Plan:
   Determine if NLRP3 antagonist co-treatment increases T cell apoptosis/deletion in response to anti-TNF.
   Determine if the level of NLRP3 RNA expression is greater in TNF-resistant Crohn's disease and ulcerative colitis samples compared to anti-TNF treatment-naïve samples.
   Determine if NLRP3 antagonist treatment decreases processed IL-1β, processed caspase-1 or secreted IL-1β, and inflammatory cytokine RNA levels.

### Biological Assay—Nigericin-Stimulated IL-1β Secretion Assay in THP-1 Cells

Monocytic THP-1 cells (ATCC: TIB-202) were maintained according to providers' instructions in RPMI media (RPMI/Hepes+10% fetal bovine serum+Sodium Pyruvate+0.05 mM Beta-mercaptoethanol (1000× stock)+Pen-Strep). Cells were differentiated in bulk with 0.5 μM phorbol 12-myristate 13-acetate (PMA; Sigma #P8139) for 3 hours, media was exchanged, and cells were plated at 50,000 cells per well in a 384-well flat-bottom cell culture plates (Greiner, #781986), and allowed to differentiate overnight. Compound in a 1:3.16 serial dilution series in DMSO was added 1:100 to the cells and incubated for 1 hour. The NLRP3 inflammasome was activated with the addition of 15 μM (final concentration) Nigericin (Enzo Life Sciences, #BML-CA421-0005), and cells were incubated for 3 hours. 10 μL supernatant was removed, and IL-1β levels were monitored using an HTRF assay (CisBio, #62IL1PEC) according to manufacturers' instructions. Viability and pyroptosis was monitored with the addition of PrestoBlue cell viability reagent (Life Technologies, #A13261) directly to the cell culture plate.

A number of embodiments of the invention have been described. Nevertheless, it will be understood that various modifications may be made without departing from the spirit and scope of the invention. Accordingly, other embodiments are within the scope of the following claims.

What is claimed is:

1. A compound of Formula AA 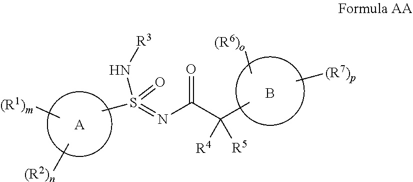
wherein
m=0, 1, or 2;
n=0, 1, or 2;
o=1 or 2;
p=0, 1, 2, or 3;
wherein
A is a 5-10-membered monocyclic or bicyclic heteroaryl or a C₆-C₁₀ monocyclic or bicyclic aryl;
B is a 5-10-membered monocyclic or bicyclic heteroaryl or a C₆-C₁₀ monocyclic or bicyclic aryl;
wherein
at least one R⁶ is ortho to the bond connecting the B ring to the C(R⁴R⁵) group of Formula AA;
R¹ and R² are each independently selected from C₁-C₆ alkyl, C₁-C₆ haloalkyl, C₁-C₆ alkoxy, C₁-C₆ haloalkoxy, halo, CN, NO₂, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NR⁸R⁹, C(O)R¹³, CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,
wherein the C₁-C₆ alkyl, C₁-C₆ alkoxy, C₁-C₆ haloalkyl, C₆-C₁₀ aryl, 5- to 10-membered heteroaryl, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, R¹⁵, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, —O(C₀-C₃ alkylene)C₆-C₁₀ aryl, halo, NR⁸R⁹, or oxo;
wherein the 3- to 7-membered heterocycloalkyl, C₆-C₁₀ aryl, and 5- to 10-membered heteroaryl are optionally substituted with one or more substituents independently selected from halo, C₁-C₆ alkyl, and OC₁-C₆ alkyl;
or one pair of R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring containing 1-3 heteroatoms independently selected from O, N, and S, wherein the carbocyclic ring or heterocyclic ring is optionally independently substituted with one or more substituents independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹,
wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, C₂-C₆ alkenyl, and C₂-C₆ alkynyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, C₃-C₁₀ cycloalkyl, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), C₆-C₁₀ aryloxy, C₃-C₁₀ cycloalkoxy, and S(O₂)C₁-C₆ alkyl; and
wherein the C₁-C₆ alkyl or C₁-C₆ alkoxy that R⁶ or R⁷ is substituted with is optionally substituted with one or more hydroxyl, C₆-C₁₀ aryl, or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, and 5- to 10-membered heteroaryl are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
each of R⁴ and R⁵ is independently selected from hydrogen and C₁-C₆ alkyl;
R¹⁰ is C₁-C₆ alkyl;
each of R⁸ and R⁹ at each occurrence is independently selected from hydrogen, C₁-C₆ alkyl, C₂-C₆ alkenyl, C₃-C₇ cycloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl, 3- to 7-membered heterocycloalkyl, or NR¹¹R¹²;
or R⁸ and R⁹ taken together with the nitrogen they are attached to form a 3- to 10-membered monocyclic or bicyclic ring optionally containing one or more heteroatoms in addition to the nitrogen they are attached to, wherein the ring is optionally substituted with one or more substituents independently selected from halo, C₁-C₆ alkyl, C₁-C₆ haloalkyl, C₁-C₆ alkoxy, oxo, N(C₁-C₆ alkyl)₂, NH₂, NH(C₁-C₆ alkyl), and hydroxy;
R¹³ is C₁-C₆ alkyl or —(Z¹-Z²)ₐ₁—Z³;
each of R¹¹ and R¹² at each occurrence is independently selected from hydrogen, C₁-C₆ alkyl, and —(Z¹-Z²)ₐ₁—Z³;
a1 is an integer selected from 0-10 (e.g., 0-5);
each Z¹ is independently C₁-C₆ alkylene optionally substituted with one or more substituents independently selected from oxo, halo, and hydroxy;
each Z² is independently a bond, NH, N(C₁-C₆ alkyl), —O—, —S—, or 5-10 membered heteroarylene;
Z³ is independently C₆-C₁₀ aryl, C₂-C₆ alkyenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆ alkyl)₂, NH₂, NH(C₁-C₆ alkyl), and hydroxy;
R³ is selected from hydrogen, cyano, hydroxy, C₁-C₆ alkoxy, C₁-C₆ alkyl, and 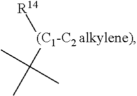
wherein the C₁-C₂ alkylene group is optionally substituted by oxo;
R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally substituted with from 1-3 independently selected R⁶;
R¹⁵ is —(Z⁴-Z⁵)ₐ₂—Z⁶;
a2 is an integer selected from 1-10 (e.g., 1-5 (e.g., 2-5));
each Z⁴ is independently selected from —O—, —S—, —NH—, and —N(C₁-C₃ alkyl)-;
provided that the Z⁴ group directly attached to R¹ or R² is —O— or —S—;
each Z⁵ is independently C₁-C₆ alkylene optionally substituted with one or more substituents independently selected from oxo, halo, and hydroxy; and
Z⁶ is OH, OC₁-C₆ alkyl, NH₂, NH(C₁-C₆ alkyl), N(C₁-C₆ alkyl)₂, NHC(O)(C₁-C₆ alkyl), NHC(O)(C₁-C₆ alkoxy), or an optionally substituted group selected from the group consisting of:
C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆ alkyl)₂, NH₂, NH(C₁-C₆ alkyl), and hydroxy;
or a pharmaceutically acceptable salt thereof.

2. A compound of Formula AA 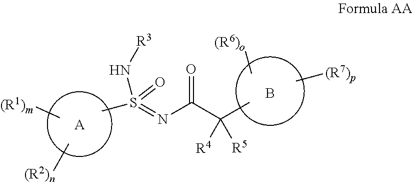
wherein
m=0, 1, or 2;
n=0, 1, or 2;
o=1 or 2;
p=0, 1, 2, or 3;
wherein
A is a 5-10-membered monocyclic or bicyclic heteroaryl or a C₆-C₁₀ monocyclic or bicyclic aryl;
B is a 5-10-membered monocyclic or bicyclic heteroaryl or a C₆-C₁₀ monocyclic or bicyclic aryl;
wherein
at least one R⁶ is ortho to the bond connecting the B ring to the C(R⁴R⁵) group of Formula AA;
R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO—C₆-C₁₀ aryl, CO-5- to 10-membered heteroaryl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,
wherein the C₁-C₆ alkyl, C₁-C₆ alkoxy, C₁-C₆ haloalkyl, C₆-C₁₀ aryl, 5- to 10-membered heteroaryl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, R¹⁵, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, —O(C₀-C₃ alkylene)C₆-C₁₀ aryl, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or one pair of R¹ and R² on adjacent atoms, taken together with the atoms connecting them, independently form one monocyclic or bicyclic C₄-C₁₂ carbocyclic ring or one monocyclic or bicyclic 5-to-12-membered heterocyclic ring containing 1-3 heteroatoms independently selected from O, N, and S, wherein the carbocyclic ring or heterocyclic ring is optionally independently substituted with one or more substituents independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, C₂-C₆ alkenyl, and C₂-C₆ alkynyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, C₃-C₁₀ cycloalkyl, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, C₃-C₁₀ cycloalkoxy, and S(O₂)C₁-C₆ alkyl; and
wherein the C₁-C₆ alkyl or C₁-C₆ alkoxy that R⁶ or R⁷ is substituted with is optionally substituted with one or more hydroxyl, C₆-C₁₀ aryl, or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
each of R⁴ and R⁵ is independently selected from hydrogen and C₁-C₆ alkyl;
R¹⁰ is C₁-C₆ alkyl;
each of R⁸ and R⁹ at each occurrence is independently selected from hydrogen, C₁-C₆ alkyl, C₂-C₆ alkenyl, C₃-C₇ cycloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;
R¹³ is C₁-C₆ alkyl, C₆-C₁₀ aryl, or 5- to 10-membered heteroaryl;
each of R¹¹ and R¹² at each occurrence is independently selected from hydrogen and C₁-C₆ alkyl;
R³ is selected from hydrogen, cyano, hydroxy, C₁-C₆ alkoxy, C₁-C₆ alkyl, and 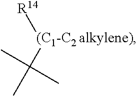
wherein the C₁-C₂ alkylene group is optionally substituted by oxo;
R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally independently substituted with from 1-3 R⁶,
R¹⁵ is —(Z⁴-Z⁵)ₐ₂—Z⁶;
a2 is an integer selected from 1-10 (e.g., 1-5 (e.g., 2-5));
each Z⁴ is independently selected from —O—, —S—, —NH—, and —N(C₁-C₃ alkyl)-;
provided that the Z⁴ group directly attached to R¹ or R² is —O— or —S—;
each Z⁵ is independently C₁-C₆ alkylene optionally substituted with one or more substituents independently selected from oxo, halo, and hydroxy; and
Z⁶ is OH, OC₁-C₆ alkyl, NH₂, NH(C₁-C₆ alkyl), N(C₁-C₆ alkyl)₂, NHC(O)(C₁-C₆ alkyl), NHC(O)(C₁-C₆ alkoxy), or an optionally substituted group selected from the group consisting of:
C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆ alkyl)₂, NH₂, NH(C₁-C₆ alkyl), and hydroxy;
or a pharmaceutically acceptable salt thereof.

3. A compound of Formula AA 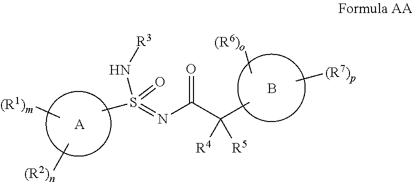
wherein
m=0, 1, or 2;
n=0, 1, or 2;
o=1 or 2;
p=0, 1, 2, or 3;
wherein
A is a 5-10-membered monocyclic or bicyclic heteroaryl or a C₆-C₁₀ monocyclic or bicyclic aryl;
B is a 5-10-membered monocyclic or bicyclic heteroaryl or a C₆-C₁₀ monocyclic or bicyclic aryl;
wherein
at least one R⁶ is ortho to the bond connecting the B ring to the C(R⁴R⁵) group of Formula AA;
R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO—C₆-C₁₀ aryl, CO-5- to 10-membered heteroaryl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl, wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹′ wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and a C₂-C₆ alkenyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl, or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
each of R⁴ and R⁵ is independently selected from hydrogen and C₁-C₆ alkyl;
R¹⁰ is C₁-C₆ alkyl;
each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;
R¹³ is C₁-C₆ alkyl, C₆-C₁₀ aryl, or 5- to 10-membered heteroaryl;
each of R¹¹ and R¹² at each occurrence is independently selected from hydrogen and C₁-C₆ alkyl;
R³ is selected from hydrogen, cyano, hydroxy, C₁-C₆ alkoxy, C₁-C₆ alkyl, and 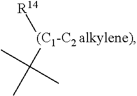
wherein the C₁-C₂ alkylene group is optionally substituted by oxo;
R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally independently substituted with 1 or 2 R⁶,
or a pharmaceutically acceptable salt thereof.

4. A compound A compound of Formula AA 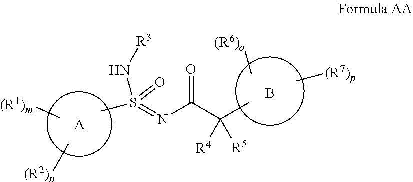
wherein
m=1 or 2;
n=1 or 2;
o=1 or 2;
p=0, 1, 2, or 3;
wherein
A is a 5-10-membered monocyclic or bicyclic heteroaryl or a C₆-C₁₀ monocyclic or bicyclic aryl;
B is a 5-10-membered monocyclic or bicyclic heteroaryl or a C₆-C₁₀ monocyclic or bicyclic aryl;
wherein
at least one R⁶ is ortho to the bond connecting the B ring to the C(R⁴R⁵) group of Formula AA;
one pair of R¹ and R² are on adjacent atoms, and taken together with the atoms connecting them, independently form one monocyclic or bicyclic C₄-C₁₂ carbocyclic ring or one monocyclic or bicyclic 5-to-12-membered heterocyclic ring containing 1-3 heteroatoms independently selected from O, N, and S, wherein the carbocyclic ring or heterocyclic ring is optionally independently substituted with one or more substituents independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹,
wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;
each of R¹ and R² that is not taken together with the atoms connecting them to form one ring is independently selected from:
C₁-C₆ alkyl, C₁-C₆ haloalkyl, C₁-C₆ alkoxy, C₁-C₆ haloalkoxy, halo, CN, NO₂, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NR⁸R⁹, C(O)R¹³, CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,
wherein the C₁-C₆ alkyl, C₁-C₆ alkoxy, C₁-C₆ haloalkyl, C₆-C₁₀ aryl, 5- to 10-membered heteroaryl, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, R¹⁵, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, —O(C₀-C₃ alkylene)C₆-C₁₀ aryl, halo, NR⁸R⁹, or oxo;
wherein the 3- to 7-membered heterocycloalkyl, C₆-C₁₀ aryl, and 5- to 10-membered heteroaryl are optionally substituted with one or more substituents independently selected from halo, C₁-C₆ alkyl, and OC₁-C₆ alkyl;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, C₂-C₆ alkenyl, and C₂-C₆ alkynyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, C₃-C₁₀ cycloalkyl, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), C₆-C₁₀ aryloxy, C₃-C₁₀ cycloalkoxy, and S(O₂)C₁-C₆ alkyl; and
wherein the C₁-C₆ alkyl or C₁-C₆ alkoxy that R⁶ or R⁷ is substituted with is optionally substituted with one or more hydroxyl, C₆-C₁₀ aryl, or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, and 5- to 10-membered heteroaryl are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
each of R⁴ and R⁵ is independently selected from hydrogen and C₁-C₆ alkyl;
R¹⁰ is C₁-C₆ alkyl;
each of R⁸ and R⁹ at each occurrence is independently selected from hydrogen, C₁-C₆ alkyl, C₂-C₆ alkenyl, C₃-C₇ cycloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl, 3- to 7-membered heterocycloalkyl, or NR¹¹R¹²;
or R⁸ and R⁹ taken together with the nitrogen they are attached to form a 3- to 10-membered monocyclic or bicyclic ring optionally containing one or more heteroatoms in addition to the nitrogen they are attached to, wherein the ring is optionally substituted with one or more substituents independently selected from halo, C₁-C₆ alkyl, C₁-C₆ haloalkyl, C₁-C₆ alkoxy, oxo, N(C₁-C₆ alkyl)₂, NH₂, NH(C₁-C₆ alkyl), and hydroxy;
R¹³ is C₁-C₆ alkyl or —(Z¹-Z²)ₐ₁—Z³;
each of R¹¹ and R¹² at each occurrence is independently selected from hydrogen, C₁-C₆ alkyl, and —(Z¹-Z²)ₐ₁—Z³;
a1 is 0-10 (e.g., 0-5);
each Z¹ is independently C₁-C₆ alkylene optionally substituted with one or more substituents independently selected from oxo, halo, and hydroxy;
each Z² is independently a bond, NH, N(C₁-C₆ alkyl), —O—, —S—, or 5-10 membered heteroarylene;
Z³ is independently C₆-C₁₀ aryl, C₂-C₆ alkyenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆ alkyl)₂, NH₂, NH(C₁-C₆ alkyl), and hydroxy;
R³ is selected from hydrogen, cyano, hydroxy, C₁-C₆ alkoxy, C₁-C₆ alkyl, and 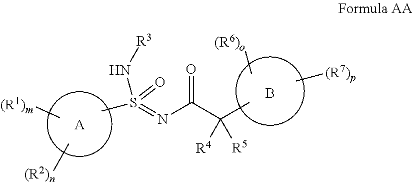
wherein the C₁-C₂ alkylene group is optionally substituted by oxo;
R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally substituted with from 1-3 independently selected R⁶,
R¹⁵ is —(Z⁴-Z⁵)ₐ₂—Z⁶;
a2 is an integer selected from 1-10 (e.g., 1-5 (e.g., 2-5));
each Z⁴ is independently selected from —O—, —S—, —NH—, and —N(C₁-C₃ alkyl)-;
provided that the Z⁴ group directly attached to R¹ or R² is —O— or —S—;
each Z⁵ is independently C₁-C₆ alkylene optionally substituted with one or more substituents independently selected from oxo, halo, and hydroxy; and
Z⁶ is OH, OC₁-C₆ alkyl, NH₂, NH(C₁-C₆ alkyl), N(C₁-C₆ alkyl)₂, NHC(O)(C₁-C₆ alkyl), NHC(O)(C₁-C₆ alkoxy), or an optionally substituted group selected from the group consisting of:
C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆ alkyl)₂, NH₂, NH(C₁-C₆ alkyl), and hydroxy;
or a pharmaceutically acceptable salt thereof.

5. A compound of Formula AA 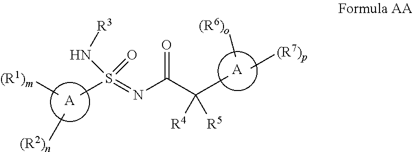
wherein
m=0, 1, or 2;
n=0, 1, or 2;
o=1 or 2;
p=0, 1, 2, or 3;
wherein
A is a 5-10-membered monocyclic or bicyclic heteroaryl or a C₆-C₁₀ monocyclic or bicyclic aryl;
B is a 5-10-membered monocyclic or bicyclic heteroaryl or a C₆-C₁₀ monocyclic or bicyclic aryl;
wherein
at least one R⁶ is ortho to the bond connecting the B ring to the C(R⁴R⁵) group of Formula AA;
one pair of R¹ and R² are on adjacent atoms, taken together with the atoms connecting them, independently form one monocyclic or bicyclic C₄-C₁₂ carbocyclic ring or one monocyclic or bicyclic 5-to-12-membered heterocyclic ring containing 1-3 heteroatoms independently selected from O, N, and S, wherein the carbocyclic ring or heterocyclic ring is optionally independently substituted with one or more substituents independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹,
wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;
each of R¹ and R² that is not taken together with the atoms connecting them to form one ring is independently selected from:
C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO—C₆-C₁₀ aryl, CO-5- to 10-membered heteroaryl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,
wherein the C₁-C₆ alkyl, C₁-C₆ alkoxy, C₁-C₆ haloalkyl, C₆-C₁₀ aryl, 5- to 10-membered heteroaryl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, R¹⁵, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, —O(C₀-C₃ alkylene)C₆-C₁₀ aryl, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, C₂-C₆ alkenyl, and C₂-C₆ alkynyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, C₃-C₁₀ cycloalkyl, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, C₃-C₁₀ cycloalkoxy, and S(O₂)C₁-C₆ alkyl; and
wherein the C₁-C₆ alkyl or C₁-C₆ alkoxy that R⁶ or R⁷ is substituted with is optionally substituted with one or more hydroxyl, C₆-C₁₀ aryl, or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
each of R⁴ and R⁵ is independently selected from hydrogen and C₁-C₆ alkyl;
R¹⁰ is C₁-C₆ alkyl;
each of R⁸ and R⁹ at each occurrence is independently selected from hydrogen, C₁-C₆ alkyl, C₂-C₆ alkenyl, C₃-C₇ cycloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;
R¹³ is C₁-C₆ alkyl, C₆-C₁₀ aryl, or 5- to 10-membered heteroaryl;
each of R¹¹ and R¹² at each occurrence is independently selected from hydrogen and C₁-C₆ alkyl;
R³ is selected from hydrogen, cyano, hydroxy, C₁-C₆ alkoxy, C₁-C₆ alkyl, and 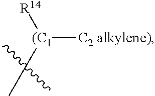
wherein the C₁-C₂ alkylene group is optionally substituted by oxo;
R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally independently substituted with from 1-3 R⁶,
R¹⁵ is —(Z⁴-Z⁵)ₐ₂—Z⁶;
a2 is an integer selected from 1-10 (e.g., 1-5 (e.g., 2-5));
each Z⁴ is independently selected from —O—, —S—, —NH—, and —N(C₁-C₃ alkyl)-;
provided that the Z⁴ group directly attached to R¹ or R² is —O— or —S—;
each Z⁵ is independently C₁-C₆ alkylene optionally substituted with one or more substituents independently selected from oxo, halo, and hydroxy; and
Z⁶ is OH, OC₁-C₆ alkyl, NH₂, NH(C₁-C₆ alkyl), N(C₁-C₆ alkyl)₂, NHC(O)(C₁-C₆ alkyl), NHC(O)(C₁-C₆ alkoxy), or an optionally substituted group selected from the group consisting of:
C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆ alkyl)₂, NH₂, NH(C₁-C₆ alkyl), and hydroxy;
or a pharmaceutically acceptable salt thereof.

6. The compound of any one of claims 4-5, wherein the compound is other than:

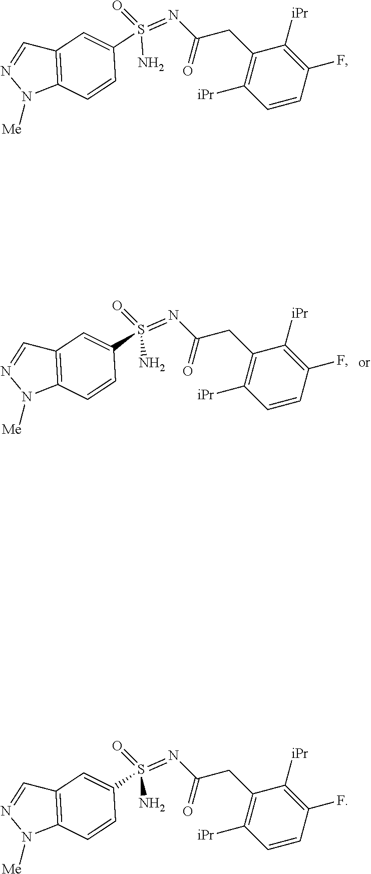 [SMILES: CC(C)c1ccc(F)c(C(C)C)c1CC(=O)N=S(N)(=O)c1ccc2c(cnn2C)c1 ; CC(C)c1ccc(F)c(C(C)C)c1CC(=O)N=[S@](N)(=O)c1ccc2c(cnn2C)c1 ; CC(C)c1ccc(F)c(C(C)C)c1CC(=O)N=[S@@](N)(=O)c1ccc2c(cnn2C)c1]

7. The compound of any one of claims 4-5, wherein one pair of R¹ and R² is on adjacent atoms, and taken together with the atoms connecting them, independently form one ring selected from:
(a) monocyclic or bicyclic C₄-C₁₂ carbocyclic ring optionally substituted with one or more substituents independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹,
wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;
(b) monocyclic or bicyclic 5-to-12-membered non-aromatic heterocyclic ring containing 1-3 heteroatoms independently selected from O, N, and S, wherein the heterocyclic ring is optionally substituted with one or more substituents independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹,
wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;
(c) monocyclic or bicyclic 6-to-12-membered aromatic heterocyclic ring containing 1-3 heteroatoms independently selected from O, N, and S, wherein the heterocyclic ring is optionally substituted with one or more substituents independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹,
wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;
(d) monocyclic 5-membered aromatic heterocyclic ring containing 2 heteroatoms independently selected from O, N, and S, wherein the heterocyclic ring is substituted with one substituent selected from hydroxy, halo, oxo, C₂-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹,
wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;
(e) monocyclic 5-membered aromatic heterocyclic ring containing 2 heteroatoms independently selected from O, N, and S, wherein the heterocyclic ring is optionally substituted with two or more substituents independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹,
wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹; and
(f) monocyclic 5-membered aromatic heterocyclic ring containing 1 or 3 heteroatoms independently selected from O, N, and S, wherein the heterocyclic ring is optionally substituted with two or more substituents independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹,
wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

8. The compound of any one of claims 1-6, wherein one pair of R¹ and R² is on adjacent atoms, and taken together with the atoms connecting them, independently form one monocyclic or bicyclic C₄-C₁₂ non-aromatic carbocyclic ring or one monocyclic or bicyclic 5-to-12-membered non-aromatic heterocyclic ring containing 1-3 heteroatoms independently selected from O, N, and S, wherein the carbocyclic ring or heterocyclic ring is optionally independently substituted with one or more substituents independently selected from hydroxy, halo, oxo, C₁-C₆ alkyl, C₂-C₆ alkenyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹′ wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, oxo, NR⁸R⁹, ═NR¹⁰, COOC₁-C₆ alkyl, C₆-C₁₀ aryl, and CONR⁸R⁹.

9. The compound of any one of claims 1-2 and 4-5, wherein when a pair of R¹ and R² on adjacent atoms, taken together with the atoms connecting them, independently form one C₄-C₈ carbocyclic ring or one 5-to-8-membered heterocyclic ring containing 1 or 2 heteroatoms independently selected from O, N, and S, then the carbocyclic ring or heterocyclic ring is independently substituted with one or more substituents each independently selected from from C₂-C₆ alkenyl, C₂-C₆ alkynyl, OC₃-C₁₀ cycloalkyl, CN, OS(O₂)C₆-C₁₀ aryl, S(O₂)C₆-C₁₀ aryl, 5- to 10-membered heteroaryl, C₃-C₁₀ cycloalkyl, and 3- to 10-membered heterocycloalkyl, wherein the S(O₂)C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

10. The compound of any one of claims 1-9, wherein A is a 5-6-membered monocyclic heteroaryl optionally substituted with 1 or 2 R¹ and optionally substituted with 1 or 2 R².

11. The compound of any one of claims 1-10, wherein A is furanyl optionally substituted with 1 or 2 R¹ and optionally substituted with 1 or 2 R².

12. The compound of any one of claims 1-10, wherein A is thiophenyl optionally substituted with 1 or 2 R¹ and optionally substituted with 1 or 2 R².

13. The compound of any one of claims 1-10, wherein A is oxazolyl optionally substituted with 1 or 2 R¹ and optionally substituted with 1 or 2 R².

14. The compound of any one of claims 1-10, wherein A is thiazolyl optionally substituted with 1 or 2 R¹ and optionally substituted with 1 or 2 R².

15. The compound of any one of claims 1-10, wherein A is pyrazolyl optionally substituted with 1 or 2 R¹ and optionally substituted with 1 or 2 R².

16. The compound of any one of claims 1-10, wherein A is imidazolyl optionally substituted with 1 or 2 R¹ and optionally substituted with 1 or 2 R².

17. The compound of any one of claims 1-9, wherein A is phenyl optionally substituted with 1 or 2 R¹ and optionally substituted with 1 or 2 R².

18. The compound of any one of claims 1-3 and 10-17, wherein m=1 and n=0.

19. The compound of any one of claims 1-3, 10, 12, and 18, wherein the substituted ring A is

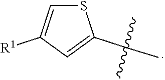

20. The compound of any one of claims 1-3, 10, 12, and 18, wherein the substituted ring A is

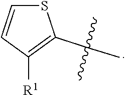

21. The compound of any one of claims 1-3, 10, 12, and 18, wherein the substituted ring A is

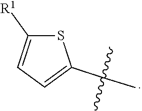

22. The compound of any one of claims 1-3, 10, 11, and 18, wherein the substituted ring A is

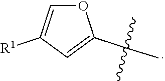

23. The compound of any one of claims 1-3, 10, 11, and 18, wherein the substituted ring A is

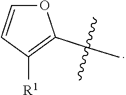

24. The compound of any one of claims 1-3, 10, 11, and 18, wherein the substituted ring A is

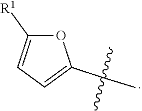

25. The compound of any one of claims 1-3, 10, 14, and 18, wherein the substituted ring A is

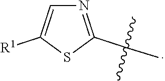

26. The compound of any one of claims 1-3, 10, 14, and 18, wherein the substituted ring A is

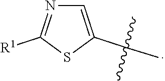

27. The compound of any one of claims 1-3, 10, 14, and 18, wherein the substituted ring A is

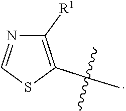

28. The compound of any one of claims 1-3, 10, 14, and 18, wherein the substituted ring A is

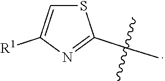

29. The compound of any one of claims 1-3, 10, 13, and 18, wherein the substituted ring A is

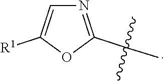

30. The compound of any one of claims 1-3, 10, 13, and 18, wherein the substituted ring A is

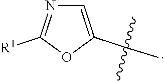

31. The compound of any one of claims 1-3, 10, 13, and 18, wherein the substituted ring A is

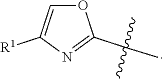

32. The compound of any one of claims 1-3 and 17-18, wherein the substituted ring A is

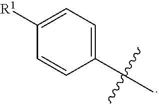

33. The compound of any one of claims 1-3 and 17-18, wherein the substituted ring A is

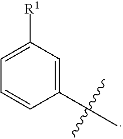

34. The compound of any one of claims 1-3 and 17-18, wherein the substituted ring A is

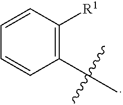

35. The compound of any one of claims 1-3, wherein the substituted ring A is

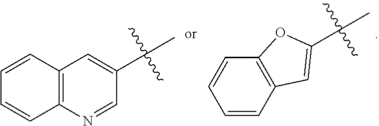 [SMILES: *c1cnc2ccccc2c1 ; *c1cc2ccccc2o1]

36. The compound of any one of claims 1-3, 10, and 15, wherein the substituted ring A is

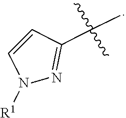

37. The compound of any one of claims 1-3, 10, and 18, wherein the substituted ring A is

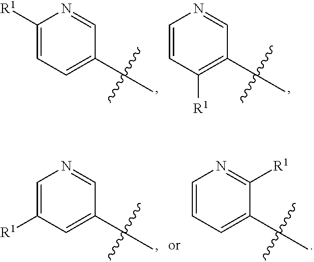

38. The compound of any one of claims 1-3, 10, and 18, wherein the substituted ring A is

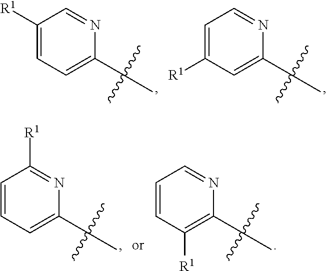

39. The compound of any one of claims 1-3, 10, and 18, wherein the substituted ring A is

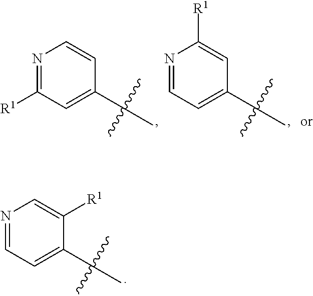

40. The compound of any one of claims 1-17, wherein m=1 and n=1.

41. The compound of any one of claims 1-10, 12, and 40, wherein the substituted ring A is

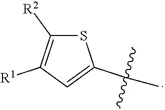

42. The compound of any one of claims 1-3, 10, 14, and 40, wherein the substituted ring A is

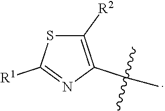

43. The compound of any one of claims 1-3, 10, 14, and 40, wherein the substituted ring A is

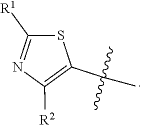

44. The compound of any one of claims 1-10, 11, and 40, wherein the substituted ring A is

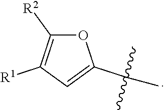

45. The compound of any one of claims 1-10, 12, and 40, wherein the substituted ring A is

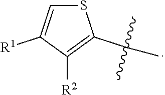

46. The compound of any one of claims 1-10, 11, and 40, wherein the substituted ring A is

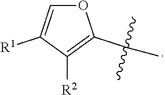

47. The compound of any one of claims 1-3, 10, 12, and 40, wherein the substituted ring A is

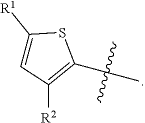

48. The compound of any one of claims 1-3, 10, 11, and 40, wherein the substituted ring A is

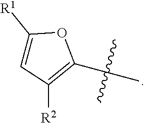

49. The compound of any one of claims 1-10, 14, and 40, wherein the substituted ring A is

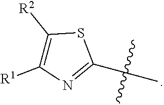

50. The compound of any one of claims 1-10, 13, and 40, wherein the substituted ring A is

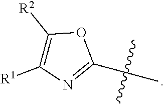

51. The compound of any one of claims 1-10, 15, and 40, wherein the optionally substituted ring A is

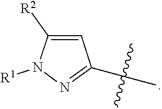

52. The compound of any one of claims 1-10, 15, and 40, wherein the optionally substituted ring A is

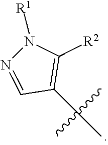

53. The compound of any one of claims 1-10, 15, and 40, wherein the optionally substituted ring A is

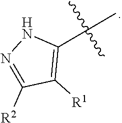

54. The compound of any one of claims 1-10, 16, and 40, wherein the optionally substituted ring A is

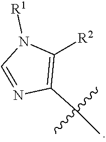

55. The compound of any one of claims 1-3, 17, and 40, wherein the substituted ring A is

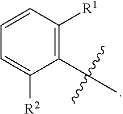

56. The compound of any one of claims 1-3, 17, and 40, wherein the substituted ring A is

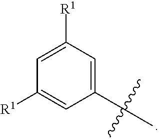

57. The compound of any one of claims 1-9, 17, and 40, wherein the substituted ring A is

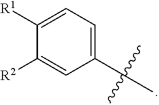

58. The compound of any one of claims 1-3, 17, and 40, wherein the substituted ring A is

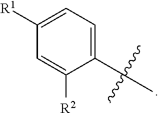

59. The compound of any one of claims 1-9, 17, and 40, wherein the substituted ring A is

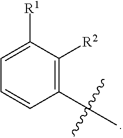

60. The compound of any one of claims 1-3, 17, and 40, wherein the substituted ring A is

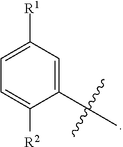

61. The compound of any one of claims 1-17, wherein m=2 and n=1.

62. The compound of any one of claims 1-3, 17, and 61, wherein the substituted ring A is

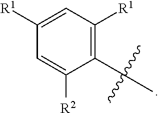

63. The compound of any one of claims 1-9, 17, and 61, wherein the substituted ring A is

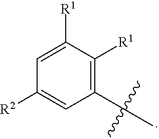

64. The compound of any one of claims 1-9, 17, and 61, wherein the substituted ring A is

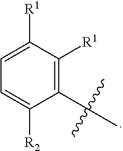

65. The compound of any one of claims 1-9, 17, and 61, wherein the substituted ring A is

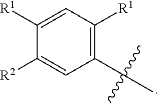

66. The compound of any one of claims 1-10, 15, and 61, wherein A is

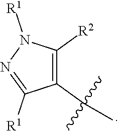

67. The compound of any one of claims 1-3 and 10-66, wherein each of R¹ and R², when present, is independently selected from the group consisting of C₁-C₆ alkyl optionally substituted with one or more hydroxy, halo, oxo, C₁-C₆ alkoxy, or NR⁸R⁹; C₃-C₇ cycloalkyl optionally substituted with one or more hydroxy, halo, oxo, C₁-C₆ alkoxy, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; 3- to 7-membered heterocycloalkyl optionally substituted with one or more hydroxy, halo, oxo, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; C₁-C₆ haloalkyl; C₁-C₆ alkoxy; C₁-C₆ haloalkoxy; halo; CN; CO—C₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl optionally substituted with one or more independently halo; 5- to 10-membered heteroaryl; NH₂; NHC₁-C₆ alkyl; N(C₁-C₆ alkyl)₂; CONR⁸R⁹; SF₅; S(O₂)NR¹¹R¹²; S(O)C₁-C₆ alkyl; and S(O₂)C₁-C₆ alkyl.

68. The compound of any one of claims 1-3 and 10-66, wherein R¹ is selected from the group consisting of 1-hydroxy-2-methylpropan-2-yl; methyl; isopropyl; 2-hydroxy-2-propyl; 1,2-dihydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; difluoromethyl; (dimethylamino)methyl; (methylamino)methyl; 1-(dimethylamino)ethyl; fluoro; chloro; phenyl; fluorophenyl; pyridyl; pyrazolyl; S(O₂)CH₃; and S(O₂)NR¹¹R¹².

69. The compound of claim 67 or 68, wherein R² is selected from the group consisting of fluoro; chloro; cyano; methyl; methoxy; ethoxy; isopropyl; 1-hydroxy-2-methylpropan-2-yl; 2-hydroxy-2-propyl; 1,2-dihydroxy-2-propyl; hydroxymethyl; 1-hydroxyethyl; 2-hydroxyethyl; 1-hydroxy-2-propyl; 1-hydroxy-1-cyclopropyl; COCH₃; COPh; 2-methoxy-2-propyl; difluoromethyl; (dimethylamino)methyl; (methylamino)methyl; S(O₂)CH₃; and S(O₂)NR¹¹R¹².

70. The compound of any one of claims 1-2, 4-5, 10-17, 40-41, 44-46, 49-54, 57, 59, 61, and 63-66, wherein one pair of R¹ and R² is on adjacent atoms, and taken together with the atoms connecting them, independently form one monocyclic or bicyclic C₄-C₁₂ carbocyclic ring (e.g., C₅ or C₆ carbocyclic ring) or one monocyclic or bicyclic 5-to-12-membered heterocyclic ring containing 1-3 (e.g., 1-2, e.g., 2) heteroatoms independently selected from O, N, and S (e.g., tetrahydropyridine, dihydrofuran, or dihydropyran), wherein the carbocyclic ring or heterocyclic ring is optionally independently substituted with one or more substituents each independently selected from hydroxy, halo, oxo, C₁-C₆ alkyl (e.g., methyl), C₂-C₆ alkenyl, C₂-C₆ alkynyl, C₁-C₆ alkoxy (e.g., methoxy, ethoxy, isopropoxyl), OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl (e.g., azetidinyl or oxetanyl), and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo (e.g., fluoro), C₁-C₆ alkyl, C₂-C₆ alkenyl, C₂-C₆ alkynyl, C₃-C₁₀ cycloalkyl, C₁-C₆ alkoxy, oxo, NR⁸R⁹ (e.g., amino, methylamino, or dimethylamino), ═NR¹⁰, COOC₁-C₆ alkyl, C₆-C₁₀ aryl, and CONR⁸R⁹.

71. The compound of any one of claims 1-2, 4-5, 10-17, 40-41, 44-46, 49-54, 57, 59, 61, and 63-66, wherein one pair of R¹ and R² is on adjacent atoms, and taken together with the atoms connecting them, independently form one monocyclic or bicyclic C₅-C₆ carbocyclic ring wherein the carbocyclic ring is optionally substituted with one or more substituents each independently selected from hydroxy, halo, oxo, methyl, isopropoxyl, azetidinyl, oxetanyl, wherein the methyl, isopropoxyl, azetidinyl, and oxetanyl are optionally substituted with one or more substituents each independently selected from hydroxy, fluoro, amino, methylamino, and dimethylamino; or
one pair of R¹ and R² on adjacent atoms taken together forms a moiety selected from: 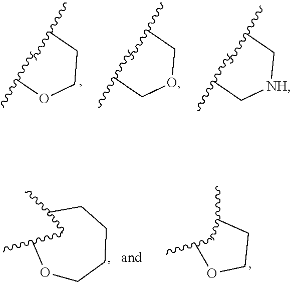
each of which is optionally independently substituted with one or more substituents each independently selected from hydroxy, halo, oxo, methyl, isopropoxyl, azetidinyl, oxetanyl, wherein the methyl, isopropoxyl, azetidinyl, and oxetanyl are optionally substituted with one or more substituents each independently selected from hydroxy, fluoro, amino, methylamino, and dimethylamino.

72. The compound of any one of claims 1-2, 4-5, 10-17, 40-41, 44-46, 49-54, 57, 59, 61, and 63-66, wherein one pair of R¹ and R² is on adjacent atoms, and taken together with the atoms connecting them, independently form at least one bicyclic spirocyclic C₄-C₁₂ carbocyclic ring, wherein the carbocyclic ring is optionally substituted with one or more substituents each independently selected from hydroxy, halo, oxo, methyl, isopropoxyl, azetidinyl, oxetanyl, wherein the methyl, isopropoxyl, azetidinyl, and oxetanyl are optionally substituted with one or more substituents each independently selected from hydroxy, fluoro, amino, methylamino, and dimethylamino.

73. The compound of any one of claims 1-2, 4-5, 10-17, 40-41, 44-46, 49-54, 57, 59, 61, and 63-66, wherein one pair of R¹ and R² is on adjacent atoms, and taken together with the atoms connecting them, independently form at least one bicyclic spirocyclic 5-to-12-membered heterocyclic ring containing 1-3 heteroatoms independently selected from O, N, and S, wherein the carbocyclic or heterocyclic ring is optionally substituted with one or more substituents each independently selected from hydroxy, halo, oxo, methyl, isopropoxyl, azetidinyl, oxetanyl, wherein the methyl, isopropoxyl, azetidinyl, and oxetanyl are optionally substituted with one or more substituents each independently selected from hydroxy, fluoro, amino, methylamino, and dimethylamino.

74. The compound of any one of claims 1-2, wherein the optionally substituted ring A is selected from the group consisting of a 5-membered heteroaryl comprising 1-3 heteroatoms independently selected from O, N, and S, wherein the heteroatom is not bonded to the position of the heteroaryl that is bonded to the S(O)(NHR³)═N moiety;
m is 1; n is 1; and
R¹ and R² are on adjacent atoms, and taken together with the atoms connecting them, independently form one monocyclic or bicyclic C₄-C₁₂ carbocyclic ring or one monocyclic or bicyclic 5-to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

75. The compound of any one of claims 1-2 and 74, wherein the optionally substituted ring A is a pyrazolyl;
m is 1; n is 1; and
R¹ and R² are on adjacent atoms, and taken together with the atoms connecting them, independently form one monocyclic or bicyclic C₄-C₁₂ carbocyclic ring or one monocyclic or bicyclic 5-to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

76. The compound of any one of claims 1-2 and 74, wherein the optionally substituted ring A is an imidazolyl;
m is 1; n is 1; and
R¹ and R² are on adjacent atoms, and taken together with the atoms connecting them, independently form one monocyclic or bicyclic C₄-C₁₂ carbocyclic ring or one monocyclic or bicyclic 5-to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

77. The compound of any one of claims 1-2 and 74, wherein the optionally substituted ring A is a thiophenyl;
m is 1; n is 1; and
R¹ and R² are on adjacent atoms, and taken together with the atoms connecting them, independently form one monocyclic or bicyclic C₄-C₁₂ carbocyclic ring or one monocyclic or bicyclic 5-to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

78. The compound of any one of claims 1-2 and 74, wherein the optionally substituted ring A is a thiazolyl;
m is 1; n is 1; and
R¹ and R² are on adjacent atoms, and taken together with the atoms connecting them, independently form one monocyclic or bicyclic C₄-C₁₂ carbocyclic ring or one monocyclic or bicyclic 5-to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

79. The compound of any one of claims 1-2, wherein the optionally substituted ring A is 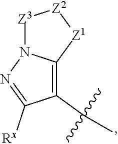
wherein R^{x} is selected from the group consisting of H and C₁-C₆ alkyl (e.g., methyl); Z¹ is selected from the group consisting of O, NH, and —CH₂— optionally substituted with 1-2 R²⁰; Z² is selected from the group consisting of NH and —CH₂— optionally substituted with 1-2 R²⁰; Z³ is selected from the group consisting of —CH₂— optionally substituted with 1-2 R²⁰, —CH₂CH₂— optionally substituted with 1-2 R²⁰, and —CH₂CH₂CH₂— optionally substituted with 1-2 R²⁰; R²⁰ is selected from the group consisting of hydroxy, halo (e.g., fluoro), oxo, C₁-C₆ alkyl (e.g., methyl or ethyl) optionally substituted with one R²¹, C₁-C₆ alkoxy (e.g., methoxy, ethoxy, or isopropoxy) optionally substituted with one R²¹, NR⁸R⁹, 3- to 10-membered heterocycloalkyl (e.g., azetidinyl or pyrrolidinyl) optionally substituted with one R²¹, or one pair of R²⁰ on the same atom, taken together with the atom connecting them, independently forms a monocyclic C₃-C₄ carbocyclic ring or a monocyclic 3- to 4-membered heterocyclic ring containing 1 O atom optionally substituted with OS(O)₂Ph; R²¹ is selected from the group consisting of halo (e.g., fluoro), NR⁸R⁹, C₂-C₆ alkynyl (e.g., ethynyl), and C₁-C₆ alkoxy (e.g., methoxy); R⁸ and R⁹ at each occurrence is independently selected from hydrogen, C₁-C₆ alkyl (e.g., methyl or ethyl), COR¹³, and CO₂R¹³; R¹³ is selected from the group consisting of: C₁-C₆ alkyl (e.g., methyl or t-butyl) and C₁-C₆ haloalkyl (e.g., trifluoromethyl).

80. The compound of any one of claims 1-2, wherein the optionally substituted ring A is 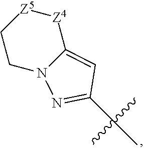
wherein Z⁴ is selected from the group consisting of —CH₂—, —C(O)—, and NH; Z⁵ is selected from the group consisting of O, NH, N—CH₃, and —CH₂—.

81. The compound of any one of the preceding claims, wherein B is phenyl substituted with 1 or 2 R⁶ and optionally substituted with 1, 2, or 3 R⁷.

82. The compound of claim 81, wherein o=2 and p=0.

83. The compound of any one of claims 81-82, wherein the substituted ring B is

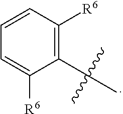

84. The compound of claim 83, wherein each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

85. The compound of any one of claims 83-84, wherein each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, wherein the C₁-C₆ alkyl, C₁-C₆ haloalkyl, and C₃-C₇ cycloalkyl is optionally substituted with one or more substituents each independently selected from hydroxy, halo, CN, and oxo.

86. The compound of claim 81, wherein o=1 and p=1.

87. The compound of claim 81, wherein o=2 and p=1.

88. The compound of claim 87, wherein the substituted ring B is

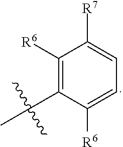

89. The compound of claim 88, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;
or R⁶ and R⁷ on adjacent atoms, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

90. The compound of claim 87, wherein the substituted ring B is

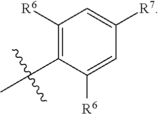

91. The compound of claim 90, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl, C₆-C₁₀ aryl, and 5- to 10-membered heteroaryl, and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy.

92. The compound of claim 81, wherein o=2 and p=2.

93. The compound of claim 92, wherein the substituted ring B is

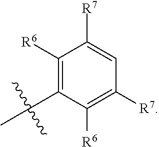

94. The compound of claim 93, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;
or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

95. The compound of claim 94, wherein each pair of R⁶ and R⁷ on adjacent atoms, taken together with the atoms connecting them, independently forms a C₄-C₇ (e.g., C₄-C₅ (e.g., C₅)) carbocyclic ring.

96. The compound of any one of claims 94-95, wherein one pair of R⁶ and R⁷ on adjacent atoms, taken together with the atoms connecting them, independently forms a C₅ carbocyclic ring.

97. The compound of claim 96, wherein the second pair of R⁶ and R⁷ on adjacent atoms, taken together with the atoms connecting them, independently forms a C₄-C₅ (e.g., C₄ or C₅) carbocyclic ring.

98. The compound of claim 94, wherein each pair of R⁶ and R⁷ on adjacent atoms, taken together with the atoms connecting them, independently forms a C₄ carbocyclic ring.

99. The compound of claim 92, wherein the substituted ring B is

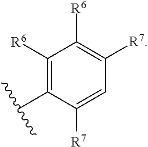

100. The compound of claim 99, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;
or R⁶ and R⁷ on adjacent atoms, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

101. The compound of claim 100, wherein R⁶ and R⁷ on adjacent atoms, taken together with the atoms connecting them, independently form C₄-C₇ (e.g., C₅) carbocyclic ring.

102. The compound of claim 100, wherein R⁶ and R⁷ on adjacent atoms, together with the atoms connecting them, independently form a 5-to-7-membered heterocyclic ring containing from 1-2 heteroatoms each independently selected from O and N (e.g., O), wherein the heterocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

103. The compound of any one of claims 101-102, wherein each of the remaining R⁶ and R⁷ is independently selected from C₁-C₆ alkyl (e.g., isopropyl).

104. The compound of claim 92, wherein the substituted ring B is

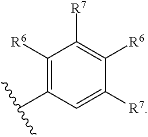

105. The compound of claim 104, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;
or R⁶ and R⁷ on adjacent atoms, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

106. The compound of any one of claims 104-105, wherein each pair of R⁶ and R⁷ on adjacent atoms, taken together with the atoms connecting them, independently forms a C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

107. The compound of claim 106, wherein each pair of R⁶ and R⁷ on adjacent atoms, taken together with the atoms connecting them, independently forms a C₄-C₇ (e.g., C₄-C₅ (e.g., C₅)) carbocyclic ring.

108. The compound of claim 81, wherein o=2 and p=3.

109. The compound of claim 108, wherein the substituted ring B is

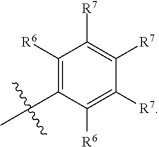

110. The compound of claim 109, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;
or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

111. The compound of any one of claims 1-80, wherein B is pyridyl; o=1 or 2; and p=0, 1, or 2.

112. The compound of claim 111, wherein o=2 and p=1.

113. The compound of claim 112, wherein the substituted ring B is

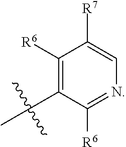

114. The compound of claim 112, wherein the substituted ring B is

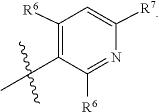

115. The compound of claim 112, wherein the substituted ring B is

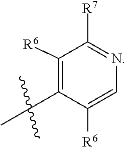

116. The compound of any one of claims 113-115, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein the C₆-C₁₀ aryl is optionally substituted with one to two C₁-C₆ alkyl optionally substituted with one to three halo; and wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;
or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

117. The compound of claim 111, wherein o=2 and p=2.

118. The compound of claim 117, wherein the substituted ring B is

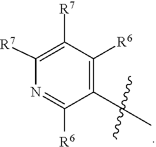

119. The compound of claim 118, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein the C₆-C₁₀ aryl is optionally substituted with one to two C₁-C₆ alkyl optionally substituted with one to three halo; and wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;
or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

120. The compound of any one of claims 1-2, wherein the optionally substituted ring A is 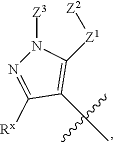
wherein R^{x} is selected from the group consisting of H and C₁-C₆ alkyl (e.g., methyl); Z¹ is selected from the group consisting of O, NH, and —CH₂— optionally substituted with 1-2 R²⁰; Z² is selected from the group consisting of NH and —CH₂— optionally substituted with 1-2 R²⁰; Z³ is selected from the group consisting of —CH₂— optionally substituted with 1-2 R²⁰, —CH₂CH₂— optionally substituted with 1-2 R²⁰, and —CH₂CH₂CH₂— optionally substituted with 1-2 R²⁰; R²⁰ is selected from the group consisting of hydroxy, halo (e.g., fluoro), oxo, C₁-C₆ alkyl (e.g., methyl or ethyl) optionally substituted with one R²¹, C₁-C₆ alkoxy (e.g., methoxy, ethoxy, or isopropoxy) optionally substituted with one R²¹, NR⁸R⁹, 3- to 10-membered heterocycloalkyl (e.g., azetidinyl or pyrrolidinyl) optionally substituted with one R²¹, or one pair of R²⁰ on the same atom, taken together with the atom connecting them, independently forms a monocyclic C₃-C₄ carbocyclic ring or a monocyclic 3- to 4-membered heterocyclic ring containing 1 O atom optionally substituted with OS(O)₂Ph; R²¹ is selected from the group consisting of halo (e.g., fluoro), NR⁸R⁹, C₂-C₆ alkynyl (e.g., ethynyl), and C₁-C₆ alkoxy (e.g., methoxy); R⁸ and R⁹ at each occurrence is independently selected from hydrogen, C₁-C₆ alkyl (e.g., methyl or ethyl), COR¹³, and CO₂R¹³; R¹³ is selected from the group consisting of: C₁-C₆ alkyl (e.g., methyl or t-butyl) and C₁-C₆ haloalkyl (e.g., trifluoromethyl); and
the substituted ring B is selected from the group consisting of: 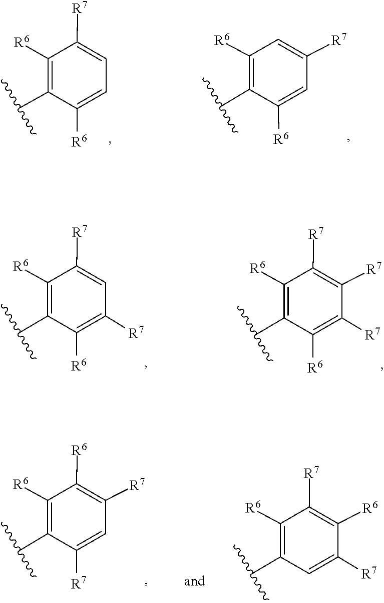
wherein
each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;
or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

121. The compound of any one of claims 1-2, wherein the optionally substituted ring A is 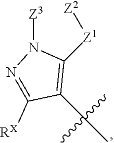
wherein R^{x} is selected from the group consisting of H and C₁-C₆ alkyl (e.g., methyl); Z is selected from the group consisting of O, NH, and —CH₂— optionally substituted with 1-2 R²⁰; Z² is selected from the group consisting of NH and —CH₂— optionally substituted with 1-2 R²⁰; Z³ is selected from the group consisting of —CH₂— optionally substituted with 1-2 R²⁰, —CH₂CH₂— optionally substituted with 1-2 R²⁰, and —CH₂CH₂CH₂— optionally substituted with 1-2 R²⁰; R²⁰ is selected from the group consisting of hydroxy, halo (e.g., fluoro), oxo, C₁-C₆ alkyl (e.g., methyl or ethyl) optionally substituted with one R²¹, C₁-C₆ alkoxy (e.g., methoxy, ethoxy, or isopropoxy) optionally substituted with one R²¹, NR⁸R⁹, 3- to 10-membered heterocycloalkyl (e.g., azetidinyl or pyrrolidinyl) optionally substituted with one R²¹, or one pair of R²⁰ on the same atom, taken together with the atom connecting them, independently forms a monocyclic C₃-C₄ carbocyclic ring or a monocyclic 3- to 4-membered heterocyclic ring containing 1 O atom optionally substituted with OS(O)₂Ph; R²¹ is selected from the group consisting of halo (e.g., fluoro), NR⁸R⁹, C₂-C₆ alkynyl (e.g., ethynyl), and C₁-C₆ alkoxy (e.g., methoxy); R⁸ and R⁹ at each occurrence is independently selected from hydrogen, C₁-C₆ alkyl (e.g., methyl or ethyl), COR¹³, and CO₂R¹³; R¹³ is selected from the group consisting of: C₁-C₆ alkyl (e.g., methyl or t-butyl) and C₁-C₆ haloalkyl (e.g., trifluoromethyl); and
the substituted ring B is selected from: 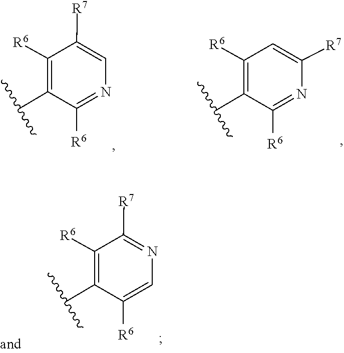
wherein
each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein the C₆-C₁₀ aryl is optionally substituted with one to two C₁-C₆ alkyl optionally substituted with one to three halo; and wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;
or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

122. The compound of any one of claims 1-2, wherein
the optionally substituted ring A is 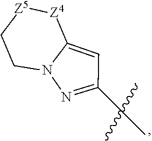
wherein Z⁴ is selected from the group consisting of —CH₂—, —C(O)—, and NH; Z⁵ is selected from the group consisting of O, NH, N—CH₃, and —CH₂—.
the substituted ring B is selected from: 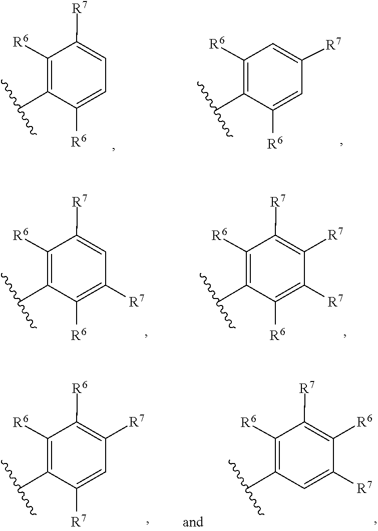
wherein
each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;
or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

123. The compound of any one of claims 1-2, wherein
the optionally substituted ring A is 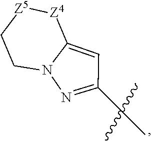
wherein Z⁴ is selected from the group consisting of —CH₂—, —C(O)—, and NH; Z⁵ is selected from the group consisting of O, NH, N—CH₃, and —CH₂—.
the substituted ring B is selected from: 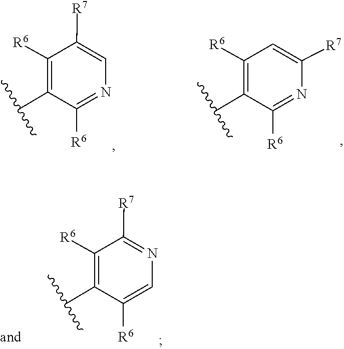
wherein
each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein the C₆-C₁₀ aryl is optionally substituted with one to two C₁-C₆ alkyl optionally substituted with one to three halo; and wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;
or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

124. The compound of any one of claims 120 and 122, wherein the substituted ring B is selected from: 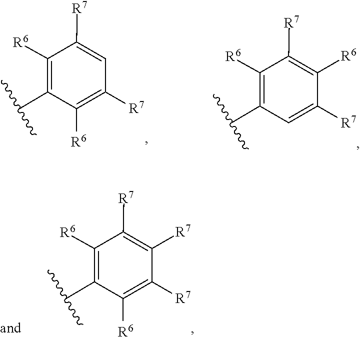
wherein each pair of R⁶ and R⁷ on adjacent atoms taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

125. The compound of any one of claims 120 and 122, wherein the substituted ring B is selected from: 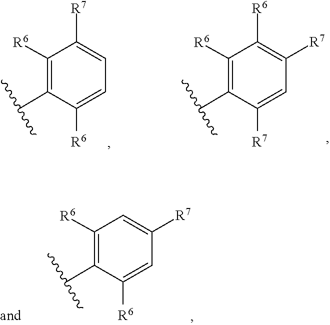
wherein each R⁶ and R⁷ is independently C₁-C₆ alkyl, C₁-C₆ haloalkyl, halo, —CN, C₃-C₇ cycloalkyl.

126. The compound of any one of claims 120 and 122, wherein the substituted ring B is: 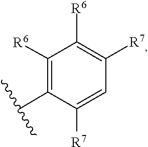
wherein
one pair of R⁶ and R⁷ on adjacent atoms taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹;
each of the remaining R⁶ and R⁷ is independently C₁-C₆ alkyl.

127. The compound of any one of the preceding claims, wherein each of R⁴ and R⁵ is hydrogen.

128. The compound of any one of the preceding claims, wherein R³ is hydrogen.

129. The compound of any one of claims 1-127, wherein R³ is cyano.

130. The compound of any one of claims 1-127, wherein R³ is 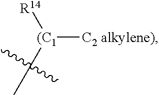
wherein the C₁-C₂ alkylene is optionally substituted by oxo.

131. The compound of claim 130, wherein R³ is CHO.

132. The compound of claim 130, wherein R³ C(O)C₁-C₆ alkyl.

133. A compound selected from the group consisting of the compounds below:

| Cmpd | Structure |
|---|---|
| 101 | 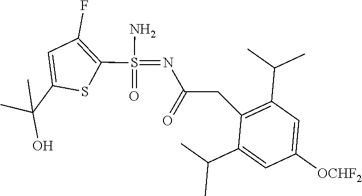 [SMILES: CC(C)c1cc(OC(F)F)cc(C(C)C)c1CC(=O)N=S(N)(=O)c1sc(C(C)(C)O)cc1F] |
| 101a | 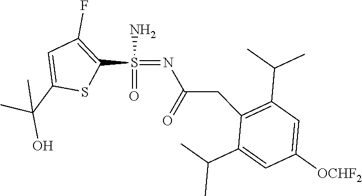 [SMILES: CC(C)c1cc(OC(F)F)cc(C(C)C)c1CC(=O)N=[S@@](N)(=O)c1sc(C(C)(C)O)cc1F] |
| 101b | 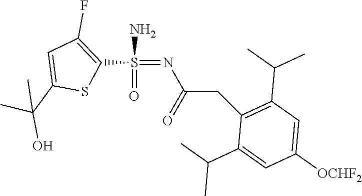 [SMILES: CC(C)c1cc(OC(F)F)cc(C(C)C)c1CC(=O)N=[S@](N)(=O)c1sc(C(C)(C)O)cc1F] |
| 102 | 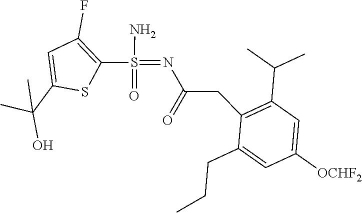 [SMILES: CCCc1cc(OC(F)F)cc(C(C)C)c1CC(=O)N=S(N)(=O)c1sc(C(C)(C)O)cc1F] |
| 103 | 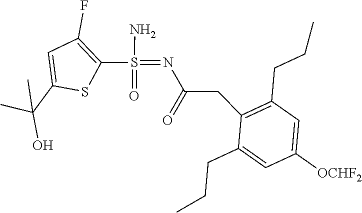 [SMILES: CCCc1cc(OC(F)F)cc(CCC)c1CC(=O)N=S(N)(=O)c1sc(C(C)(C)O)cc1F] |
| 104 | 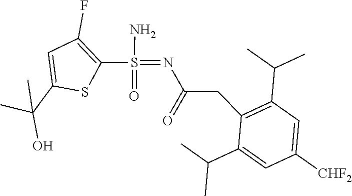 [SMILES: CC(C)c1cc(C(F)F)cc(C(C)C)c1CC(=O)N=S(N)(=O)c1sc(C(C)(C)O)cc1F] |
| 105 | 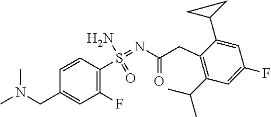 [SMILES: CC(C)c1cc(F)cc(C2CC2)c1CC(=O)N=S(N)(=O)c1ccc(CN(C)C)cc1F] |
| 106 | 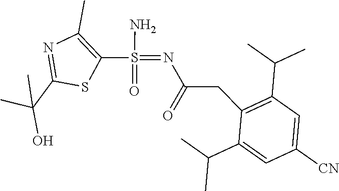 [SMILES: Cc1nc(C(C)(C)O)sc1S(N)(=O)=NC(=O)Cc1c(C(C)C)cc(C#N)cc1C(C)C] |
| 107 | 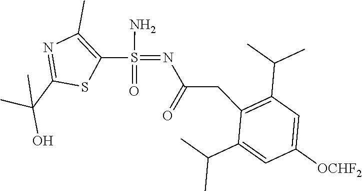 [SMILES: Cc1nc(C(C)(C)O)sc1S(N)(=O)=NC(=O)Cc1c(C(C)C)cc(OC(F)F)cc1C(C)C] |
| 108 | 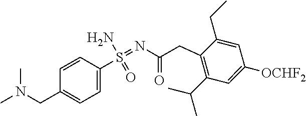 [SMILES: CCc1cc(OC(F)F)cc(C(C)C)c1CC(=O)N=S(N)(=O)c1ccc(CN(C)C)cc1] |
| 109 | 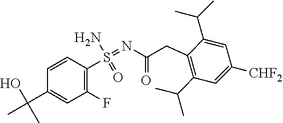 [SMILES: CC(C)c1cc(C(F)F)cc(C(C)C)c1CC(=O)N=S(N)(=O)c1ccc(C(C)(C)O)cc1F] |
| 110 | 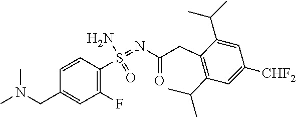 [SMILES: CC(C)c1cc(C(F)F)cc(C(C)C)c1CC(=O)N=S(N)(=O)c1ccc(CN(C)C)cc1F] |
| 111 | 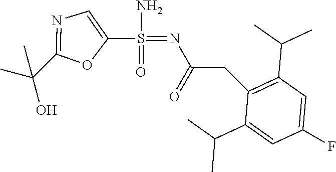 [SMILES: CC(C)c1cc(F)cc(C(C)C)c1CC(=O)N=S(N)(=O)c1cnc(C(C)(C)O)o1] |
| 112 | 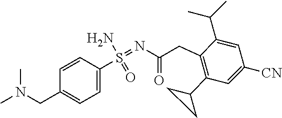 [SMILES: CC(C)c1cc(C#N)cc(C2CC2)c1CC(=O)N=S(N)(=O)c1ccc(CN(C)C)cc1] |
| 113 | 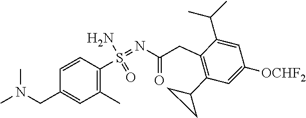 [SMILES: Cc1cc(CN(C)C)ccc1S(N)(=O)=NC(=O)Cc1c(C(C)C)cc(OC(F)F)cc1C1CC1] |
| 114 | 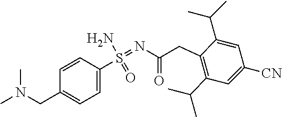 [SMILES: CC(C)c1cc(C#N)cc(C(C)C)c1CC(=O)N=S(N)(=O)c1ccc(CN(C)C)cc1] |
| 115 | 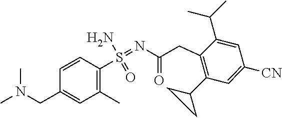 [SMILES: Cc1cc(CN(C)C)ccc1S(N)(=O)=NC(=O)Cc1c(C(C)C)cc(C#N)cc1C1CC1] |
| 116 | 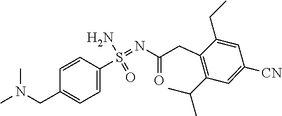 [SMILES: CCc1cc(C#N)cc(C(C)C)c1CC(=O)N=S(N)(=O)c1ccc(CN(C)C)cc1] |
| 117 | 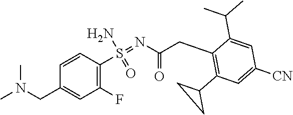 [SMILES: CC(C)c1cc(C#N)cc(C2CC2)c1CC(=O)N=S(N)(=O)c1ccc(CN(C)C)cc1F] |
| 118 | 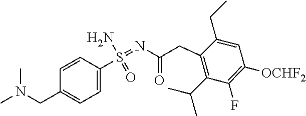 [SMILES: CCc1cc(OC(F)F)c(F)c(C(C)C)c1CC(=O)N=S(N)(=O)c1ccc(CN(C)C)cc1] |
| 119 | 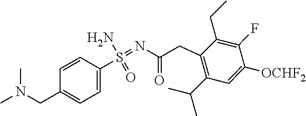 [SMILES: CCc1c(F)c(OC(F)F)cc(C(C)C)c1CC(=O)N=S(N)(=O)c1ccc(CN(C)C)cc1] |
| 120 | 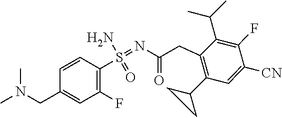 [SMILES: CC(C)c1c(F)c(C#N)cc(C2CC2)c1CC(=O)N=S(N)(=O)c1ccc(CN(C)C)cc1F] |
| 121 | 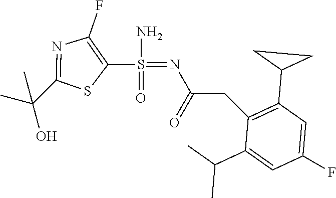 [SMILES: CC(C)c1cc(F)cc(C2CC2)c1CC(=O)N=S(N)(=O)c1sc(C(C)(C)O)nc1F] |
| 122 | 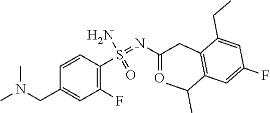 [SMILES: CCc1cc(F)cc(C(C)C)c1CC(=O)N=S(N)(=O)c1ccc(CN(C)C)cc1F] |
| 123 | 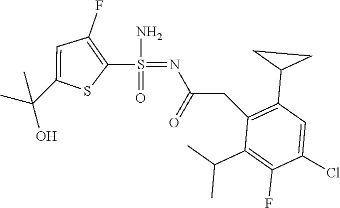 [SMILES: CC(C)c1c(F)c(Cl)cc(C2CC2)c1CC(=O)N=S(N)(=O)c1sc(C(C)(C)O)cc1F] |
| 124 | 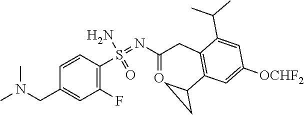 [SMILES: CC(C)c1cc(OC(F)F)cc(C2CC2)c1CC(=O)N=S(N)(=O)c1ccc(CN(C)C)cc1F] |
| 125 | 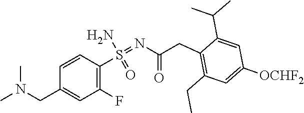 [SMILES: CCc1cc(OC(F)F)cc(C(C)C)c1CC(=O)N=S(N)(=O)c1ccc(CN(C)C)cc1F] |
| 126 | 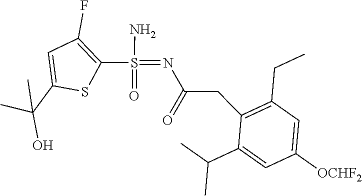 [SMILES: CCc1cc(OC(F)F)cc(C(C)C)c1CC(=O)N=S(N)(=O)c1sc(C(C)(C)O)cc1F] |
| 127 | 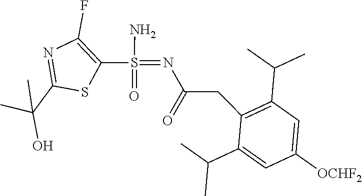 [SMILES: CC(C)c1cc(OC(F)F)cc(C(C)C)c1CC(=O)N=S(N)(=O)c1sc(C(C)(C)O)nc1F] |
| 128 | 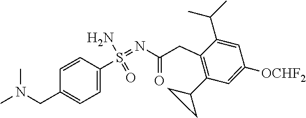 [SMILES: CC(C)c1cc(OC(F)F)cc(C2CC2)c1CC(=O)N=S(N)(=O)c1ccc(CN(C)C)cc1] |
| 129 | 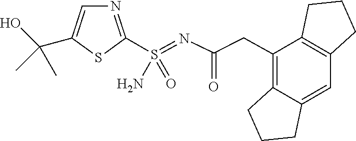 [SMILES: CC(C)(O)c1cnc(S(N)(=O)=NC(=O)Cc2c3c(cc4c2CCC4)CCC3)s1] |
| 129a | 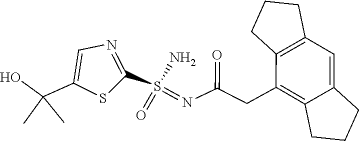 [SMILES: CC(C)(O)c1cnc([S@](N)(=O)=NC(=O)Cc2c3c(cc4c2CCC4)CCC3)s1] |
| 129b | 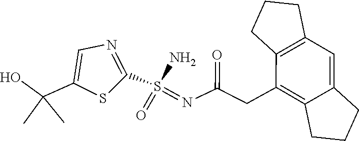 [SMILES: CC(C)(O)c1cnc([S@@](N)(=O)=NC(=O)Cc2c3c(cc4c2CCC4)CCC3)s1] |
| 130 | 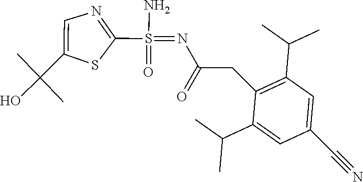 [SMILES: CC(C)c1cc(C#N)cc(C(C)C)c1CC(=O)N=S(N)(=O)c1ncc(C(C)(C)O)s1] |
| 131 | 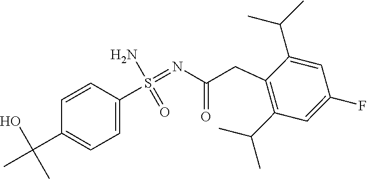 [SMILES: CC(C)c1cc(F)cc(C(C)C)c1CC(=O)N=S(N)(=O)c1ccc(C(C)(C)O)cc1] |
| 131a | 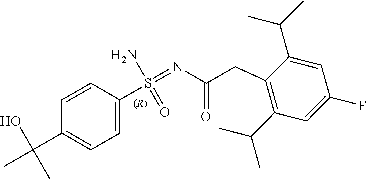 |
| 131b | 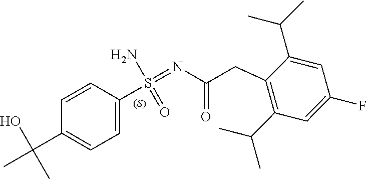 |
| 132 | 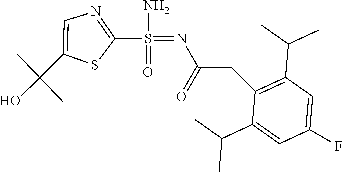 [SMILES: CC(C)c1cc(F)cc(C(C)C)c1CC(=O)N=S(N)(=O)c1ncc(C(C)(C)O)s1] |
| 132a | 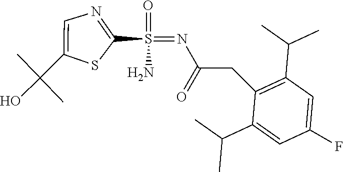 [SMILES: CC(C)c1cc(F)cc(C(C)C)c1CC(=O)N=[S@](N)(=O)c1ncc(C(C)(C)O)s1] |
| 132b | 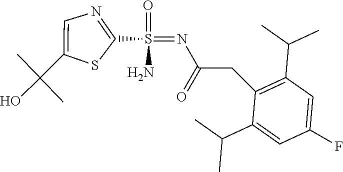 [SMILES: CC(C)c1cc(F)cc(C(C)C)c1CC(=O)N=[S@@](N)(=O)c1ncc(C(C)(C)O)s1] |
| 133 | 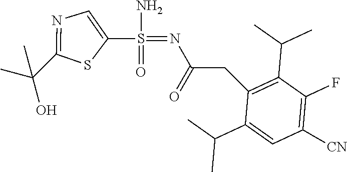 [SMILES: CC(C)c1cc(C#N)c(F)c(C(C)C)c1CC(=O)N=S(N)(=O)c1cnc(C(C)(C)O)s1] |
| 133a | 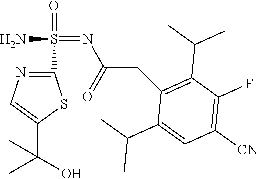 [SMILES: CC(C)c1cc(C#N)c(F)c(C(C)C)c1CC(=O)N=[S@@](N)(=O)c1ncc(C(C)(C)O)s1] |
| 133b | 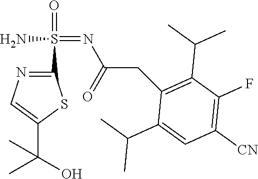 [SMILES: CC(C)c1cc(C#N)c(F)c(C(C)C)c1CC(=O)N=[S@](N)(=O)c1ncc(C(C)(C)O)s1] |
| 134 | 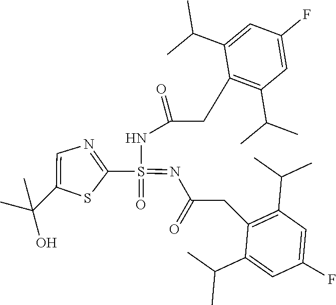 [SMILES: CC(C)c1cc(F)cc(C(C)C)c1CC(=O)N=S(=O)(NC(=O)Cc1c(C(C)C)cc(F)cc1C(C)C)c1ncc(C(C)(C)O)s1] |
| 135 | 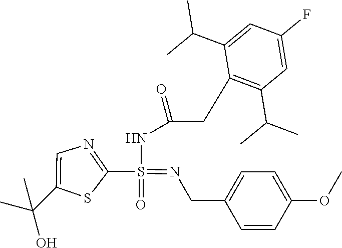 [SMILES: COc1ccc(CN=S(=O)(NC(=O)Cc2c(C(C)C)cc(F)cc2C(C)C)c2ncc(C(C)(C)O)s2)cc1] |
| 136 | 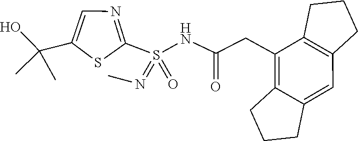 [SMILES: CN=S(=O)(NC(=O)Cc1c2c(cc3c1CCC3)CCC2)c1ncc(C(C)(C)O)s1] |
| 137 | 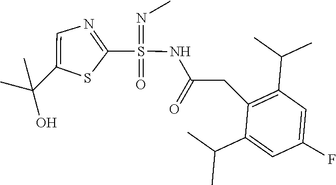 [SMILES: CN=S(=O)(NC(=O)Cc1c(C(C)C)cc(F)cc1C(C)C)c1ncc(C(C)(C)O)s1] |
| 138 | 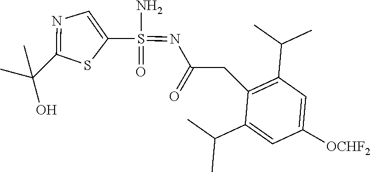 [SMILES: CC(C)c1cc(OC(F)F)cc(C(C)C)c1CC(=O)N=S(N)(=O)c1cnc(C(C)(C)O)s1] |
| 139 | 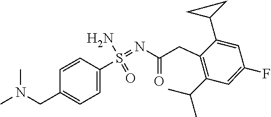 [SMILES: CC(C)c1cc(F)cc(C2CC2)c1CC(=O)N=S(N)(=O)c1ccc(CN(C)C)cc1] |
| 140 | 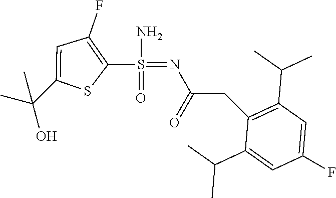 [SMILES: CC(C)c1cc(F)cc(C(C)C)c1CC(=O)N=S(N)(=O)c1sc(C(C)(C)O)cc1F] |
| 141 | 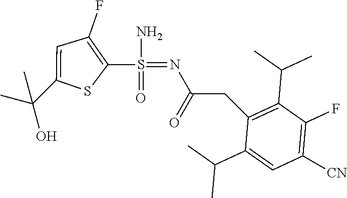 [SMILES: CC(C)c1cc(C#N)c(F)c(C(C)C)c1CC(=O)N=S(N)(=O)c1sc(C(C)(C)O)cc1F] |

and pharmaceutically acceptable salts thereof.

134. A compound selected from the group consisting of the compounds below:

| Cmpd | Structure |
|---|---|
| 101 | 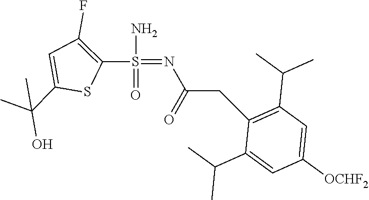 [SMILES: CC(C)c1cc(OC(F)F)cc(C(C)C)c1CC(=O)N=S(N)(=O)c1sc(C(C)(C)O)cc1F] |
| 101a | 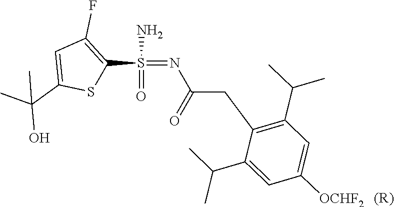 [SMILES: CC(C)c1cc(OC(F)F)cc(C(C)C)c1CC(=O)N=[S@@](N)(=O)c1sc(C(C)(C)O)cc1F ; "(R)"] |
| 101b | 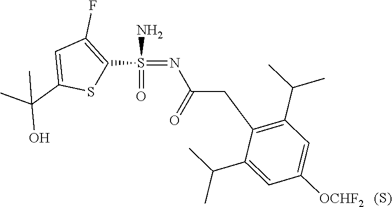 [SMILES: CC(C)c1cc(OC(F)F)cc(C(C)C)c1CC(=O)N=[S@](N)(=O)c1sc(C(C)(C)O)cc1F ; "(S)"] |
| 102 | 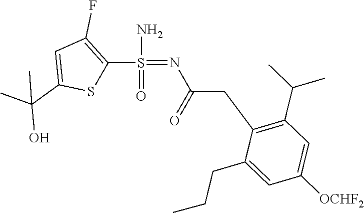 [SMILES: CCCc1cc(OC(F)F)cc(C(C)C)c1CC(=O)N=S(N)(=O)c1sc(C(C)(C)O)cc1F] |
| 103 | 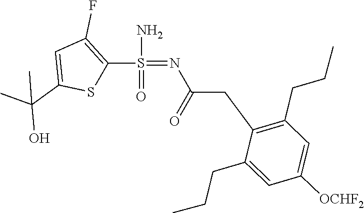 [SMILES: CCCc1cc(OC(F)F)cc(CCC)c1CC(=O)N=S(N)(=O)c1sc(C(C)(C)O)cc1F] |
| 104 | 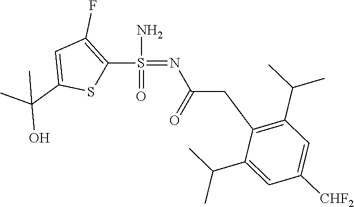 [SMILES: CC(C)c1cc(C(F)F)cc(C(C)C)c1CC(=O)N=S(N)(=O)c1sc(C(C)(C)O)cc1F] |
| 104a | 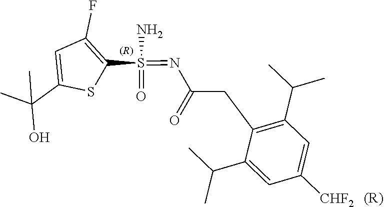 |
| 104b | 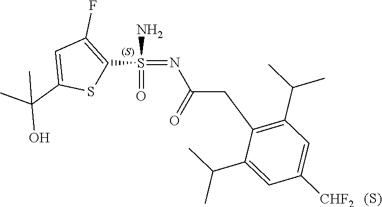 |
| 105 | 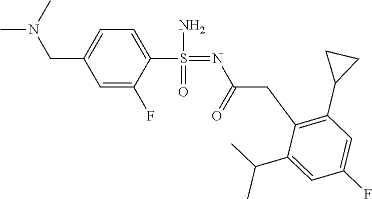 [SMILES: CC(C)c1cc(F)cc(C2CC2)c1CC(=O)N=S(N)(=O)c1ccc(CN(C)C)cc1F] |
| 106 | 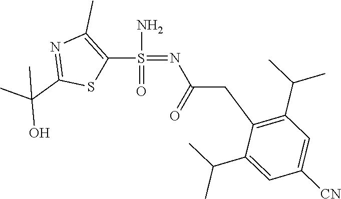 [SMILES: Cc1nc(C(C)(C)O)sc1S(N)(=O)=NC(=O)Cc1c(C(C)C)cc(C#N)cc1C(C)C] |
| 106a | 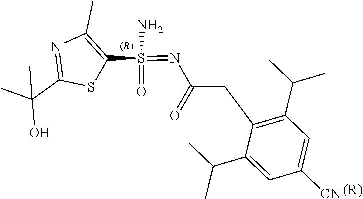 |
| 106b | 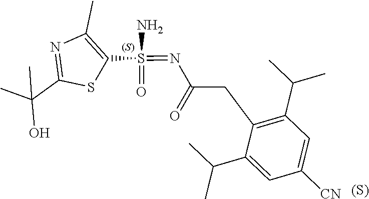 |
| 107 | 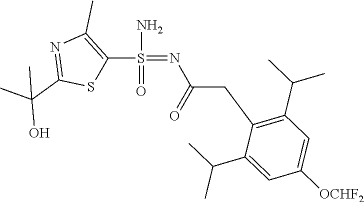 [SMILES: Cc1nc(C(C)(C)O)sc1S(N)(=O)=NC(=O)Cc1c(C(C)C)cc(OC(F)F)cc1C(C)C] |
| 107a | 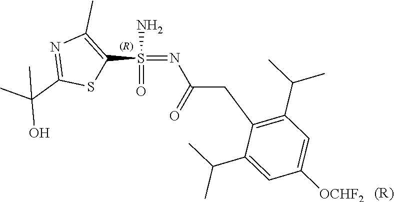 |
| 107b | 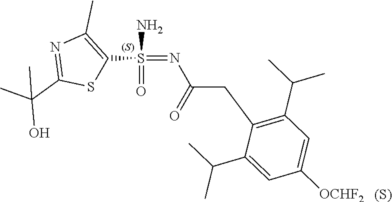 |
| 108 | 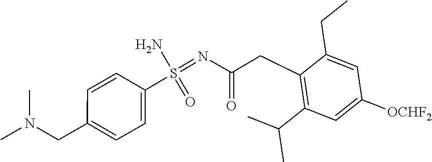 [SMILES: CCc1cc(OC(F)F)cc(C(C)C)c1CC(=O)N=S(N)(=O)c1ccc(CN(C)C)cc1] |
| 109 | 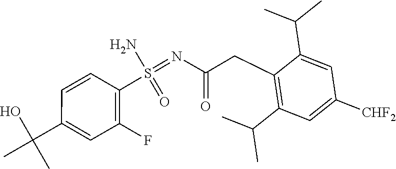 [SMILES: CC(C)c1cc(C(F)F)cc(C(C)C)c1CC(=O)N=S(N)(=O)c1ccc(C(C)(C)O)cc1F] |
| 110 | 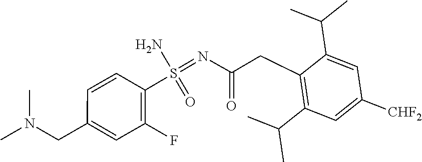 [SMILES: CC(C)c1cc(C(F)F)cc(C(C)C)c1CC(=O)N=S(N)(=O)c1ccc(CN(C)C)cc1F] |
| 110a | 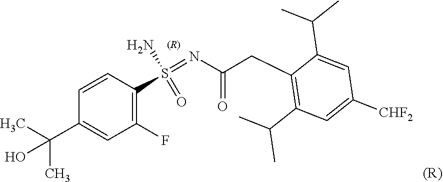 |
| 110b | 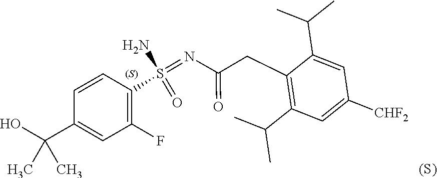 |
| 111 | 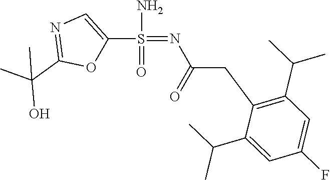 [SMILES: CC(C)c1cc(F)cc(C(C)C)c1CC(=O)N=S(N)(=O)c1cnc(C(C)(C)O)o1] |
| 112 | 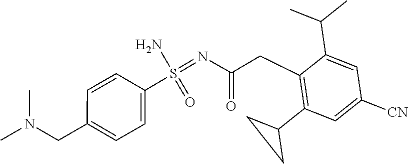 [SMILES: CC(C)c1cc(C#N)cc(C2CC2)c1CC(=O)N=S(N)(=O)c1ccc(CN(C)C)cc1] |
| 112a | 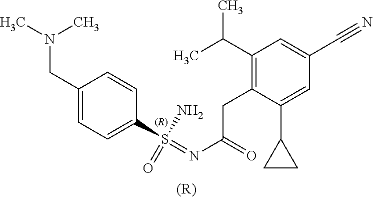 |
| 112b | 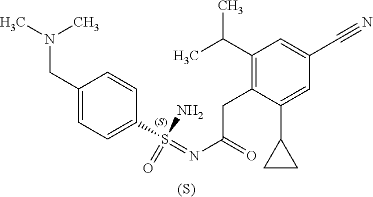 |
| 113 | 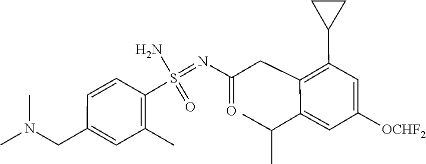 [SMILES: Cc1cc(CN(C)C)ccc1S(N)(=O)=NC(=O)Cc1c(C(C)C)cc(OC(F)F)cc1C1CC1] |
| 114 | 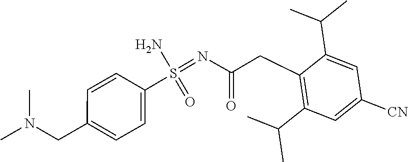 [SMILES: CC(C)c1cc(C#N)cc(C(C)C)c1CC(=O)N=S(N)(=O)c1ccc(CN(C)C)cc1] |
| 114a | 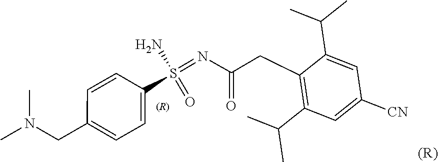 |
| 114b | 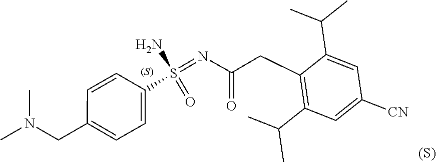 |
| 115 | 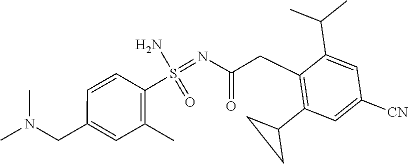 [SMILES: Cc1cc(CN(C)C)ccc1S(N)(=O)=NC(=O)Cc1c(C(C)C)cc(C#N)cc1C1CC1] |
| 116 | 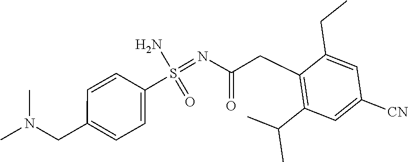 [SMILES: CCc1cc(C#N)cc(C(C)C)c1CC(=O)N=S(N)(=O)c1ccc(CN(C)C)cc1] |
| 116a | 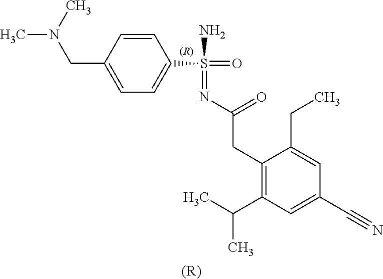 |
| 116b | 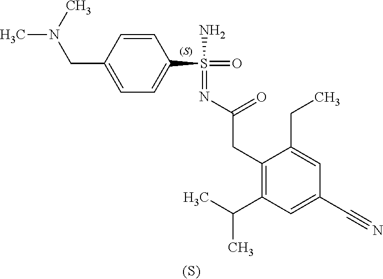 |
| 117 | 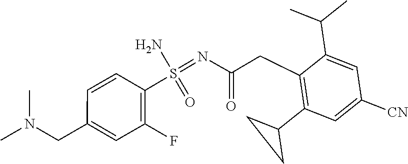 [SMILES: CC(C)c1cc(C#N)cc(C2CC2)c1CC(=O)N=S(N)(=O)c1ccc(CN(C)C)cc1F] |
| 117a | 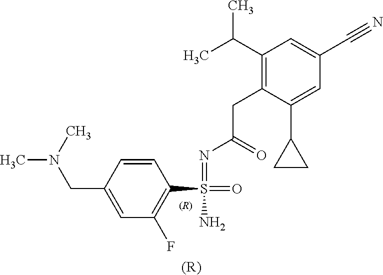 |
| 117b | 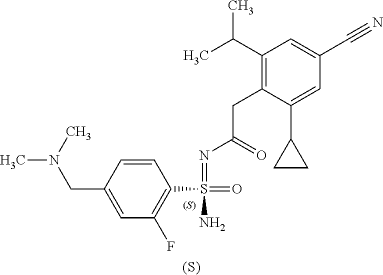 |
| 118 | 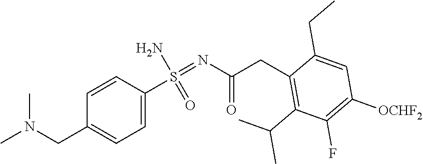 [SMILES: CCc1cc(OC(F)F)c(F)c(C(C)C)c1CC(=O)N=S(N)(=O)c1ccc(CN(C)C)cc1] |
| 119 | 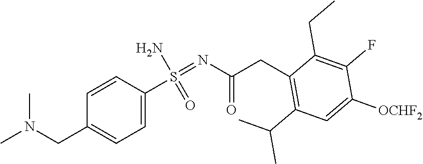 [SMILES: CCc1c(F)c(OC(F)F)cc(C(C)C)c1CC(=O)N=S(N)(=O)c1ccc(CN(C)C)cc1] |
| 120 | 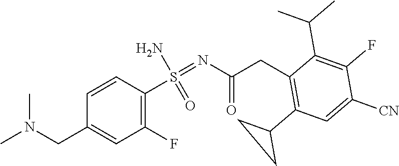 [SMILES: CC(C)c1c(F)c(C#N)cc(C2CC2)c1CC(=O)N=S(N)(=O)c1ccc(CN(C)C)cc1F] |
| 121 | 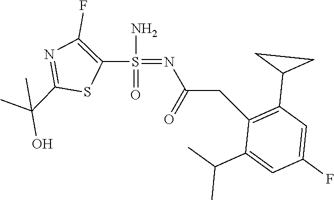 [SMILES: CC(C)c1cc(F)cc(C2CC2)c1CC(=O)N=S(N)(=O)c1sc(C(C)(C)O)nc1F] |
| 122 | 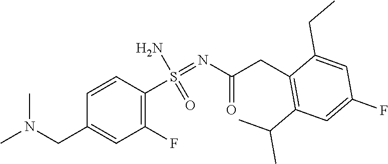 [SMILES: CCc1cc(F)cc(C(C)C)c1CC(=O)N=S(N)(=O)c1ccc(CN(C)C)cc1F] |
| 123 | 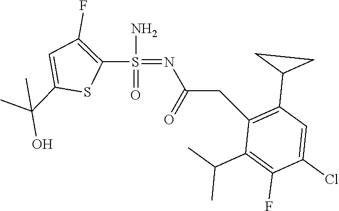 [SMILES: CC(C)c1c(F)c(Cl)cc(C2CC2)c1CC(=O)N=S(N)(=O)c1sc(C(C)(C)O)cc1F] |
| 123a | 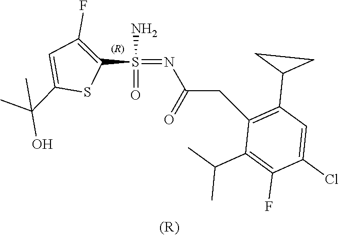 |
| 123b | 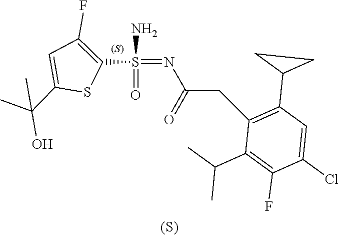 |
| 124 | 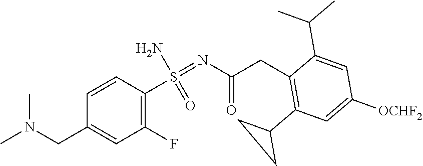 [SMILES: CC(C)c1cc(OC(F)F)cc(C2CC2)c1CC(=O)N=S(N)(=O)c1ccc(CN(C)C)cc1F] |
| 125 | 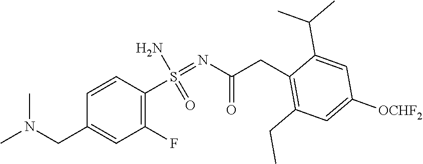 [SMILES: CCc1cc(OC(F)F)cc(C(C)C)c1CC(=O)N=S(N)(=O)c1ccc(CN(C)C)cc1F] |
| 126 | 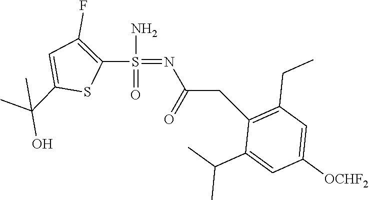 [SMILES: CCc1cc(OC(F)F)cc(C(C)C)c1CC(=O)N=S(N)(=O)c1sc(C(C)(C)O)cc1F] |
| 126a | 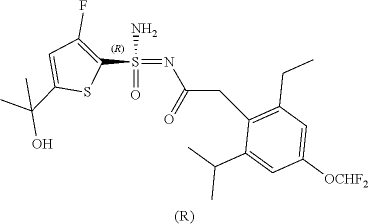 |
| 126b | 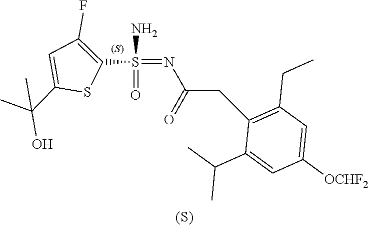 |
| 127 | 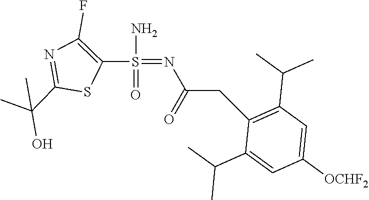 [SMILES: CC(C)c1cc(OC(F)F)cc(C(C)C)c1CC(=O)N=S(N)(=O)c1sc(C(C)(C)O)nc1F] |
| 128 | 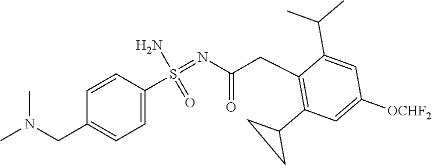 [SMILES: CC(C)c1cc(OC(F)F)cc(C2CC2)c1CC(=O)N=S(N)(=O)c1ccc(CN(C)C)cc1] |
| 129 | 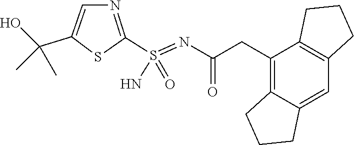 [SMILES: CC(C)(O)c1cnc(S(N)(=O)=NC(=O)Cc2c3c(cc4c2CCC4)CCC3)s1] |
| 129a | 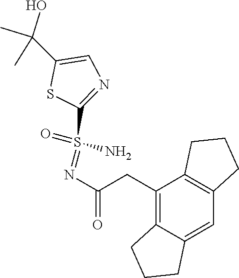 [SMILES: CC(C)(O)c1cnc([S@](N)(=O)=NC(=O)Cc2c3c(cc4c2CCC4)CCC3)s1] |
| 129b | 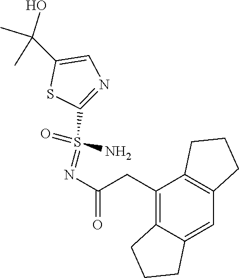 [SMILES: CC(C)(O)c1cnc([S@@](N)(=O)=NC(=O)Cc2c3c(cc4c2CCC4)CCC3)s1] |
| 130 | 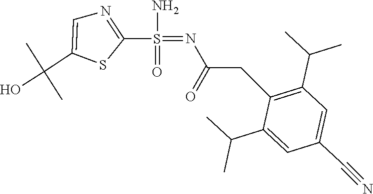 [SMILES: CC(C)c1cc(C#N)cc(C(C)C)c1CC(=O)N=S(N)(=O)c1ncc(C(C)(C)O)s1] |
| 130a | 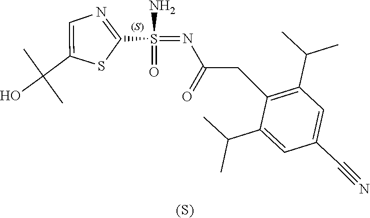 |
| 130b | 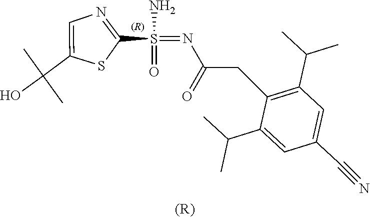 |
| 131 | 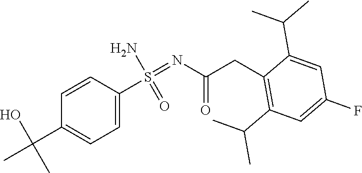 [SMILES: CC(C)c1cc(F)cc(C(C)C)c1CC(=O)N=S(N)(=O)c1ccc(C(C)(C)O)cc1] |
| 131a | 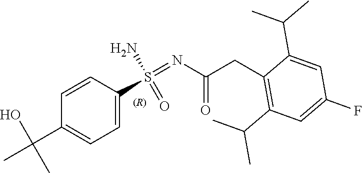 |
| 131b | 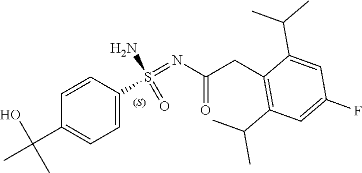 |
| 132 | 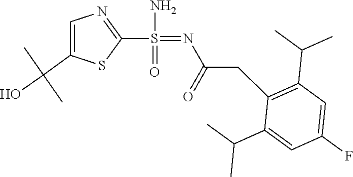 [SMILES: CC(C)c1cc(F)cc(C(C)C)c1CC(=O)N=S(N)(=O)c1ncc(C(C)(C)O)s1] |
| 132a | 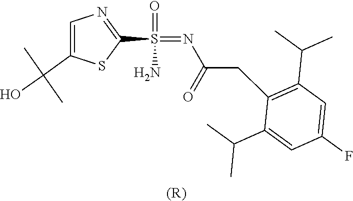 [SMILES: CC(C)c1cc(F)cc(C(C)C)c1CC(=O)N=[S@](N)(=O)c1ncc(C(C)(C)O)s1 ; "(R)"] |
| 132b | 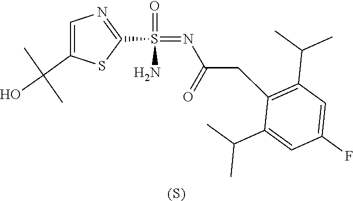 [SMILES: CC(C)c1cc(F)cc(C(C)C)c1CC(=O)N=[S@@](N)(=O)c1ncc(C(C)(C)O)s1 ; "(S)"] |
| 133 | 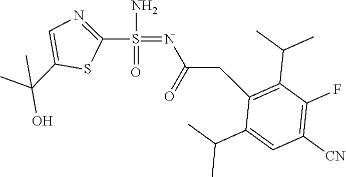 [SMILES: CC(C)c1cc(C#N)c(F)c(C(C)C)c1CC(=O)N=S(N)(=O)c1ncc(C(C)(C)O)s1] |
| 133a | 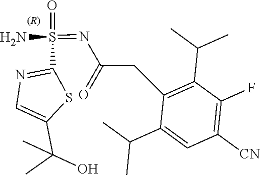 |
| 133b | 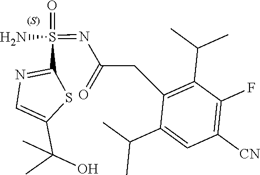 |
| 134 | 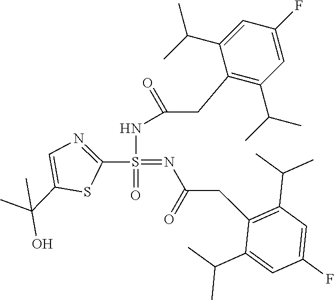 [SMILES: CC(C)c1cc(F)cc(C(C)C)c1CC(=O)N=S(=O)(NC(=O)Cc1c(C(C)C)cc(F)cc1C(C)C)c1ncc(C(C)(C)O)s1] |
| 135 | 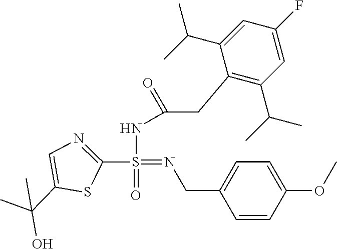 [SMILES: COc1ccc(CN=S(=O)(NC(=O)Cc2c(C(C)C)cc(F)cc2C(C)C)c2ncc(C(C)(C)O)s2)cc1] |
| 136 | 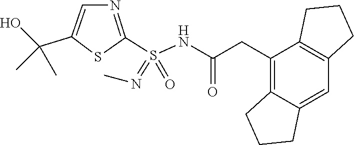 [SMILES: CN=S(=O)(NC(=O)Cc1c2c(cc3c1CCC3)CCC2)c1ncc(C(C)(C)O)s1] |
| 137 | 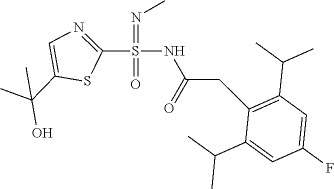 [SMILES: CN=S(=O)(NC(=O)Cc1c(C(C)C)cc(F)cc1C(C)C)c1ncc(C(C)(C)O)s1] |
| 138 | 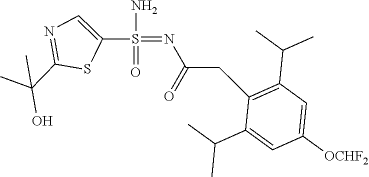 [SMILES: CC(C)c1cc(OC(F)F)cc(C(C)C)c1CC(=O)N=S(N)(=O)c1cnc(C(C)(C)O)s1] |
| 138a | 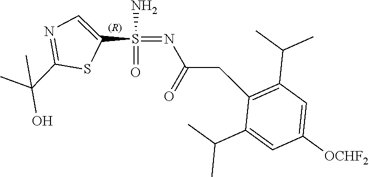 |
| 138b | 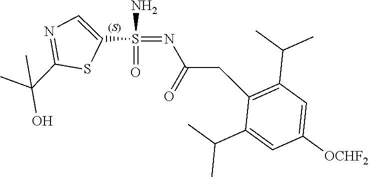 |
| 139 | 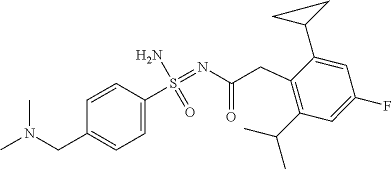 [SMILES: CC(C)c1cc(F)cc(C2CC2)c1CC(=O)N=S(N)(=O)c1ccc(CN(C)C)cc1] |
| 139a | 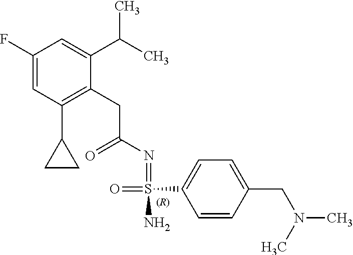 |
| 139b | 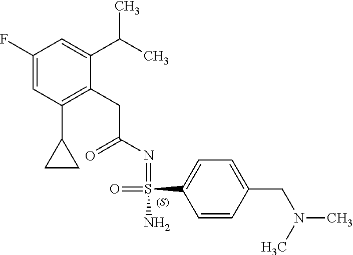 |
| 140 | 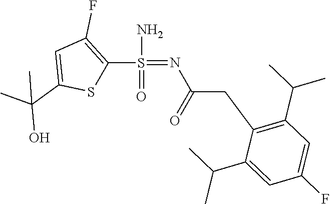 [SMILES: CC(C)c1cc(F)cc(C(C)C)c1CC(=O)N=S(N)(=O)c1sc(C(C)(C)O)cc1F] |
| 140a | 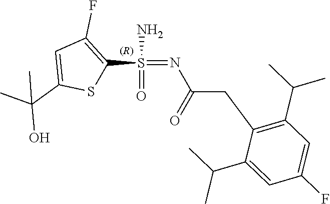 |
| 140b | 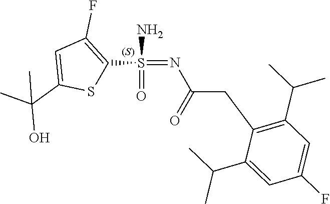 |
| 141 | 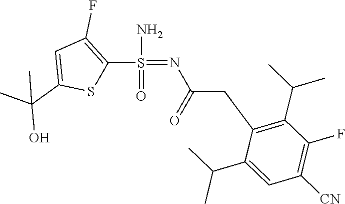 [SMILES: CC(C)c1cc(C#N)c(F)c(C(C)C)c1CC(=O)N=S(N)(=O)c1sc(C(C)(C)O)cc1F] |
| 141a | 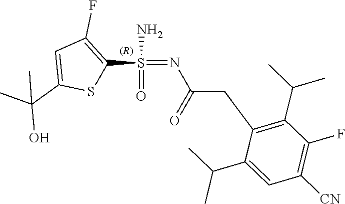 |
| 141b | 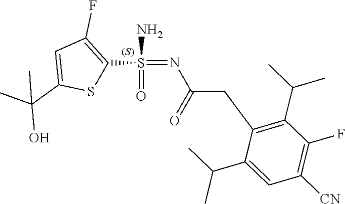 |
| 142 | 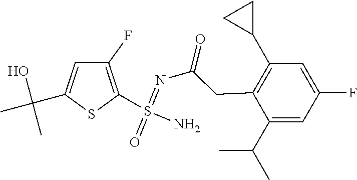 [SMILES: CC(C)c1cc(F)cc(C2CC2)c1CC(=O)N=S(N)(=O)c1sc(C(C)(C)O)cc1F] |
| 143 | 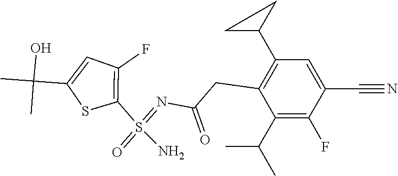 [SMILES: CC(C)c1c(F)c(C#N)cc(C2CC2)c1CC(=O)N=S(N)(=O)c1sc(C(C)(C)O)cc1F] |
| 144 | 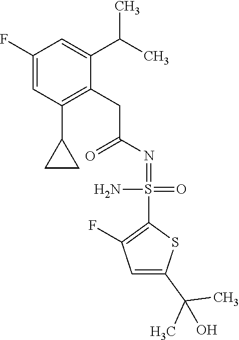 [SMILES: CC(C)c1cc(F)cc(C2CC2)c1CC(=O)N=S(N)(=O)c1sc(C(C)(C)O)cc1F] |
| 144a | 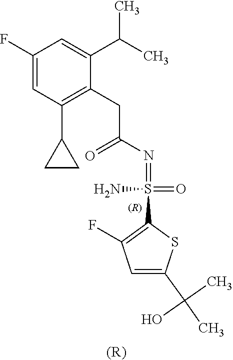 |
| 144b | 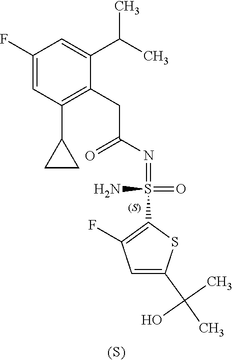 |
| 145 | 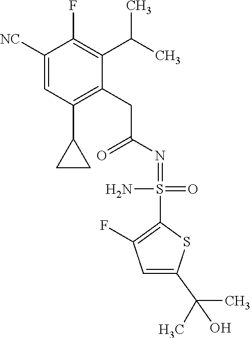 [SMILES: CC(C)c1c(F)c(C#N)cc(C2CC2)c1CC(=O)N=S(N)(=O)c1sc(C(C)(C)O)cc1F] |
| 145a | 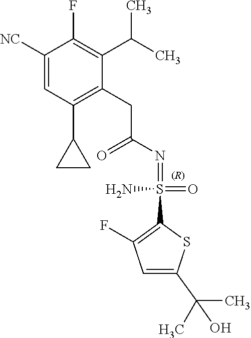 |
| 145b | (S) |
| 146 | |
| 147 | 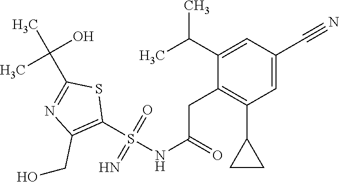 [SMILES: CC(C)c1cc(C#N)cc(C2CC2)c1CC(=O)NS(=N)(=O)c1sc(C(C)(C)O)nc1CO] |
| 147a | 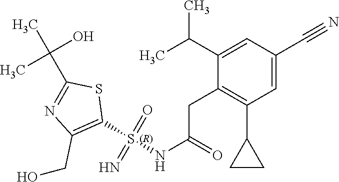 |
| 147b | 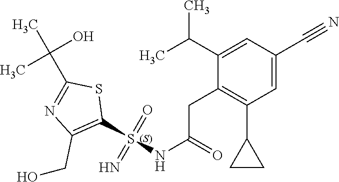 |
| 148 | 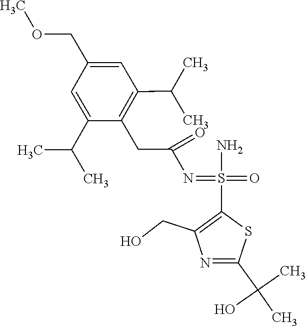 [SMILES: COCc1cc(C(C)C)c(CC(=O)N=S(N)(=O)c2sc(C(C)(C)O)nc2CO)c(C(C)C)c1] |
| 148a | 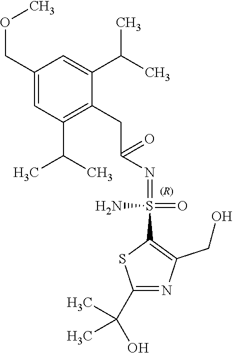 |
| 148b | 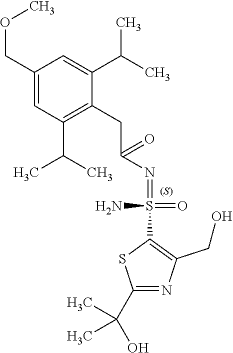 |
| 149a | 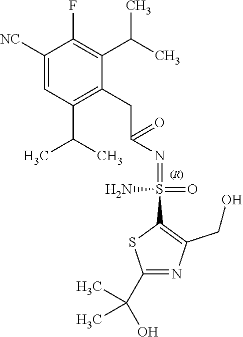 |
| 149b | |
| 150 | 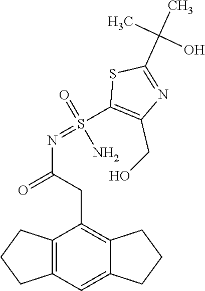 [SMILES: CC(C)(O)c1nc(CO)c(S(N)(=O)=NC(=O)Cc2c3c(cc4c2CCC4)CCC3)s1] |
| 150a | 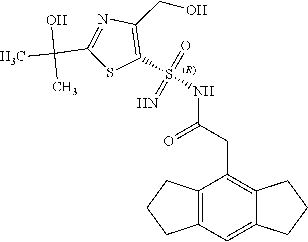 |
| 150b | 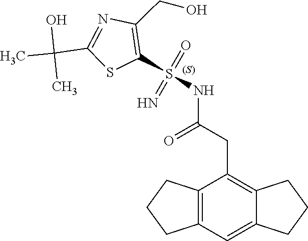 |
| 151 | |
| 152 | 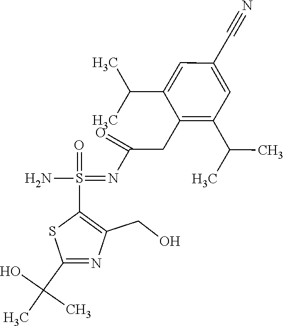 [SMILES: CC(C)c1cc(C#N)cc(C(C)C)c1CC(=O)N=S(N)(=O)c1sc(C(C)(C)O)nc1CO] |
| 152a | 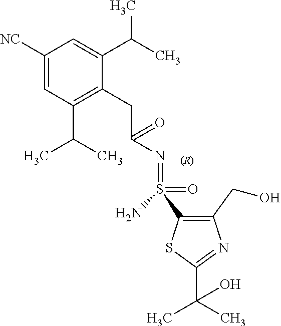 |
| 152b | 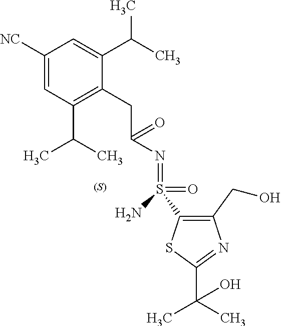 |
| 153 | 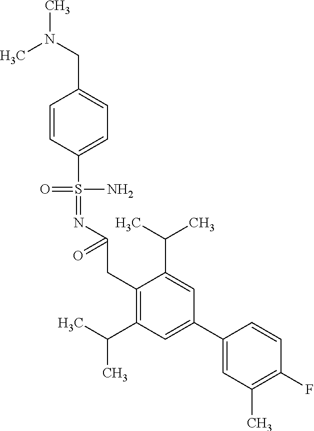 [SMILES: Cc1cc(-c2cc(C(C)C)c(CC(=O)N=S(N)(=O)c3ccc(CN(C)C)cc3)c(C(C)C)c2)ccc1F] |
| 154 | 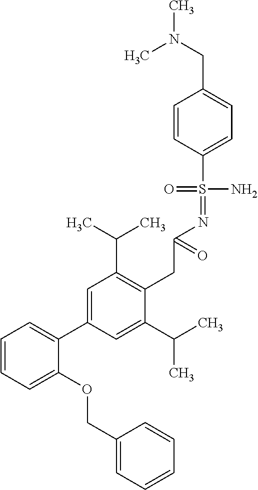 [SMILES: CC(C)c1cc(-c2ccccc2OCc2ccccc2)cc(C(C)C)c1CC(=O)N=S(N)(=O)c1ccc(CN(C)C)cc1] |
| 155 | 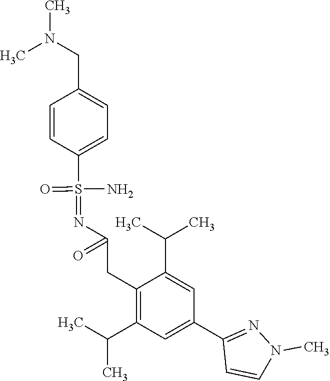 |
| 158 | 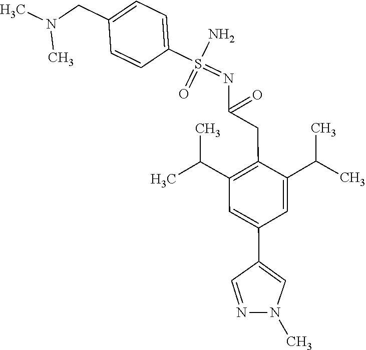 [SMILES: CC(C)c1cc(-c2cnn(C)c2)cc(C(C)C)c1CC(=O)N=S(N)(=O)c1ccc(CN(C)C)cc1] |
| 159 | 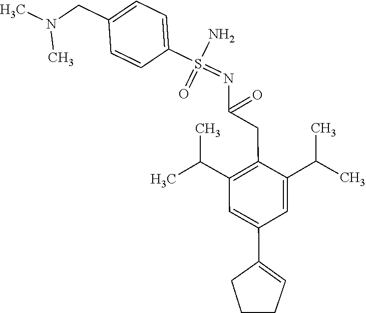 [SMILES: CC(C)c1cc(C2=CCCC2)cc(C(C)C)c1CC(=O)N=S(N)(=O)c1ccc(CN(C)C)cc1] |
| 160 | 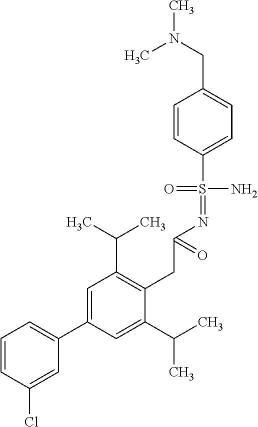 [SMILES: CC(C)c1cc(-c2cccc(Cl)c2)cc(C(C)C)c1CC(=O)N=S(N)(=O)c1ccc(CN(C)C)cc1] |
| 161 | 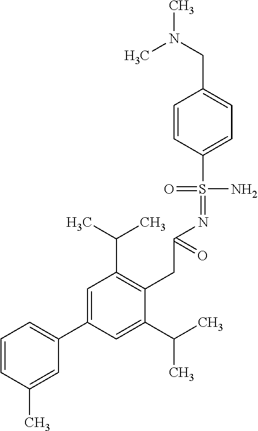 [SMILES: Cc1cccc(-c2cc(C(C)C)c(CC(=O)N=S(N)(=O)c3ccc(CN(C)C)cc3)c(C(C)C)c2)c1] |
| 162 | 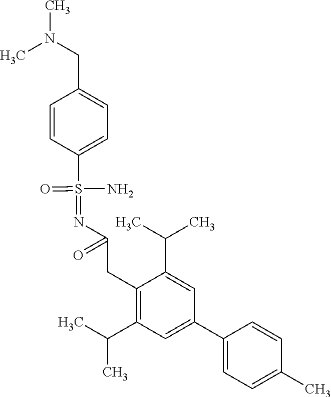 |
| 163 | 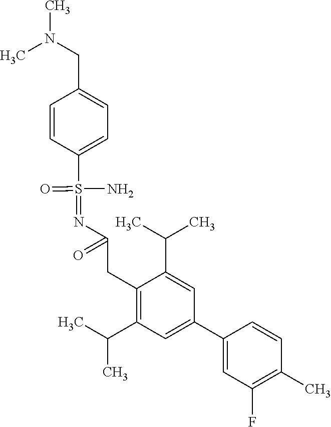 |
| 164 | 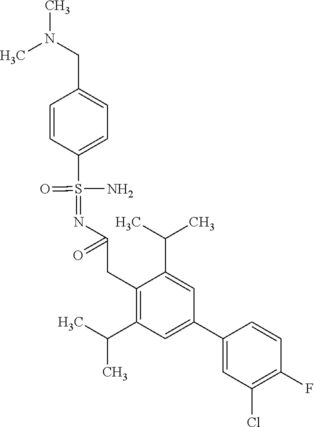 [SMILES: CC(C)c1cc(-c2ccc(F)c(Cl)c2)cc(C(C)C)c1CC(=O)N=S(N)(=O)c1ccc(CN(C)C)cc1] |
| 165 | 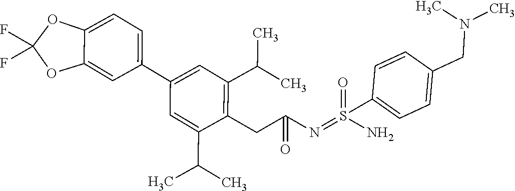 [SMILES: CC(C)c1cc(-c2ccc3c(c2)OC(F)(F)O3)cc(C(C)C)c1CC(=O)N=S(N)(=O)c1ccc(CN(C)C)cc1] |
| 168 | 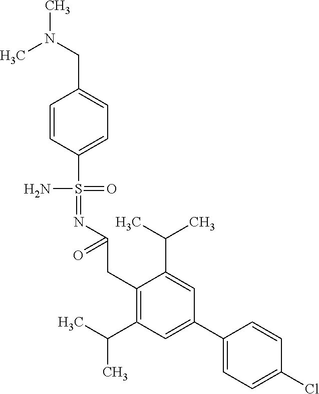 [SMILES: CC(C)c1cc(-c2ccc(Cl)cc2)cc(C(C)C)c1CC(=O)N=S(N)(=O)c1ccc(CN(C)C)cc1] |
| 169 | 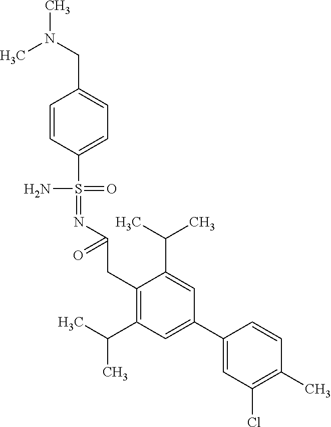 |
| 170 | 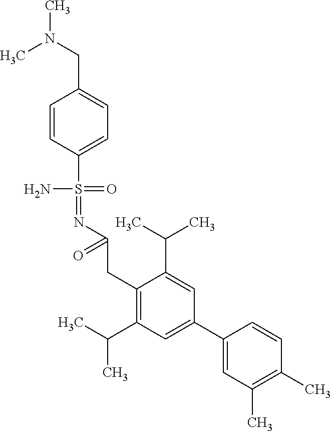 |
| 171 | 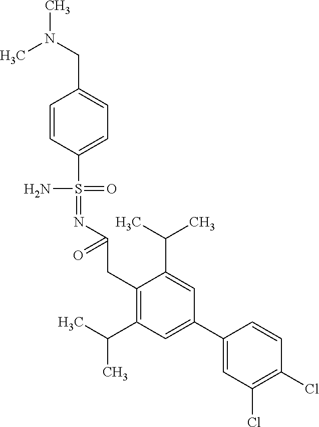 [SMILES: CC(C)c1cc(-c2ccc(Cl)c(Cl)c2)cc(C(C)C)c1CC(=O)N=S(N)(=O)c1ccc(CN(C)C)cc1] |
| 172 | 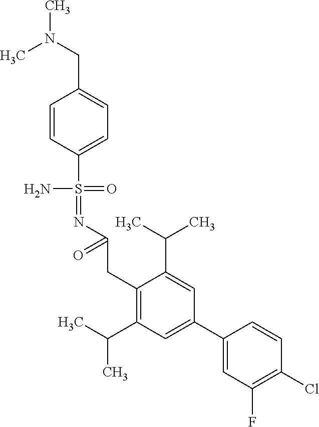 [SMILES: CC(C)c1cc(-c2ccc(Cl)c(F)c2)cc(C(C)C)c1CC(=O)N=S(N)(=O)c1ccc(CN(C)C)cc1] |
| 173 | 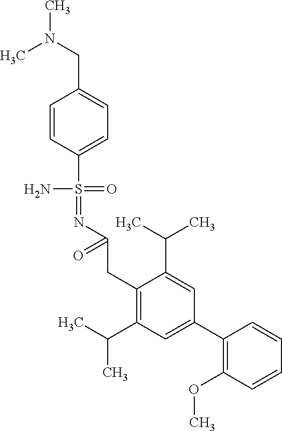 [SMILES: COc1ccccc1-c1cc(C(C)C)c(CC(=O)N=S(N)(=O)c2ccc(CN(C)C)cc2)c(C(C)C)c1] |
| 176 | 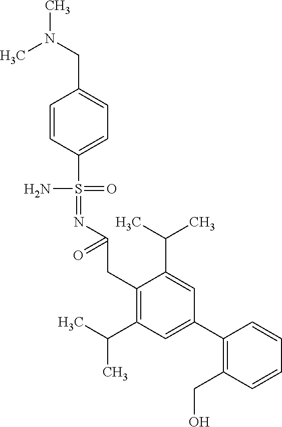 [SMILES: CC(C)c1cc(-c2ccccc2CO)cc(C(C)C)c1CC(=O)N=S(N)(=O)c1ccc(CN(C)C)cc1] |
| 177 | 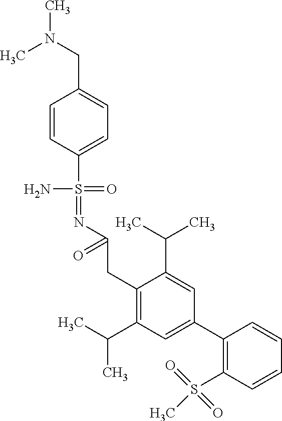 [SMILES: CC(C)c1cc(-c2ccccc2S(C)(=O)=O)cc(C(C)C)c1CC(=O)N=S(N)(=O)c1ccc(CN(C)C)cc1] |
| 178 | 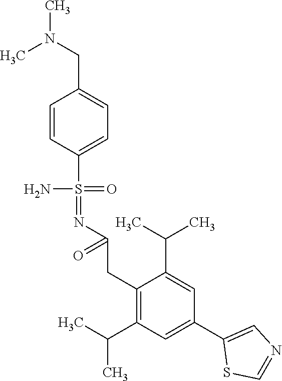 [SMILES: CC(C)c1cc(-c2cncs2)cc(C(C)C)c1CC(=O)N=S(N)(=O)c1ccc(CN(C)C)cc1] |
| 180 | 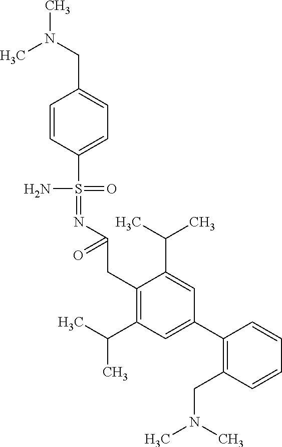 [SMILES: CC(C)c1cc(-c2ccccc2CN(C)C)cc(C(C)C)c1CC(=O)N=S(N)(=O)c1ccc(CN(C)C)cc1] |
| 181 | 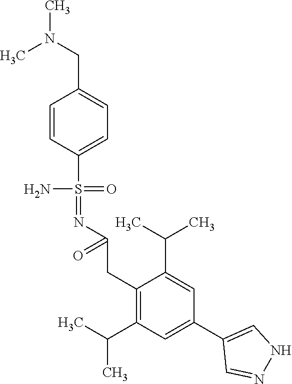 [SMILES: CC(C)c1cc(-c2cn[nH]c2)cc(C(C)C)c1CC(=O)N=S(N)(=O)c1ccc(CN(C)C)cc1] |
| 182 | 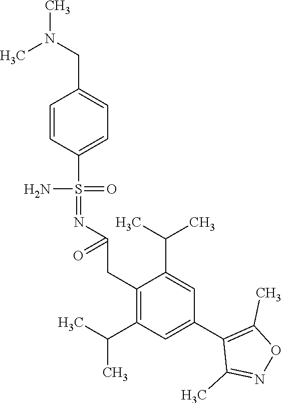 [SMILES: Cc1noc(C)c1-c1cc(C(C)C)c(CC(=O)N=S(N)(=O)c2ccc(CN(C)C)cc2)c(C(C)C)c1] |
| 183 | 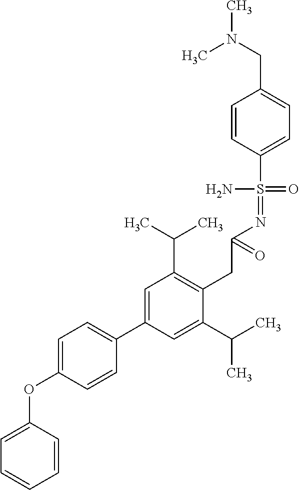 [SMILES: CC(C)c1cc(-c2ccc(Oc3ccccc3)cc2)cc(C(C)C)c1CC(=O)N=S(N)(=O)c1ccc(CN(C)C)cc1] |
| 184 | 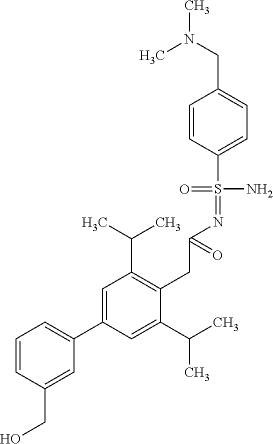 [SMILES: CC(C)c1cc(-c2cccc(CO)c2)cc(C(C)C)c1CC(=O)N=S(N)(=O)c1ccc(CN(C)C)cc1] |
| 185 | 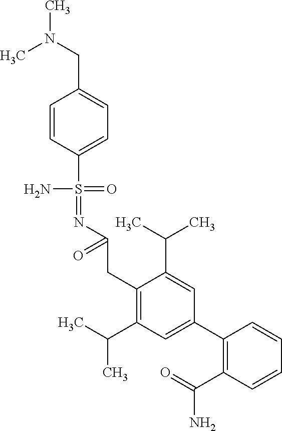 [SMILES: CC(C)c1cc(-c2ccccc2C(N)=O)cc(C(C)C)c1CC(=O)N=S(N)(=O)c1ccc(CN(C)C)cc1] |
| 186 | 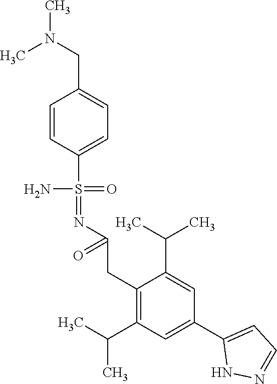 [SMILES: CC(C)c1cc(-c2ccn[nH]2)cc(C(C)C)c1CC(=O)N=S(N)(=O)c1ccc(CN(C)C)cc1] |
| 189 | 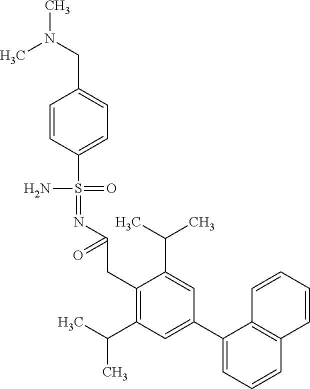 [SMILES: CC(C)c1cc(-c2cccc3ccccc23)cc(C(C)C)c1CC(=O)N=S(N)(=O)c1ccc(CN(C)C)cc1] |
| 190 | 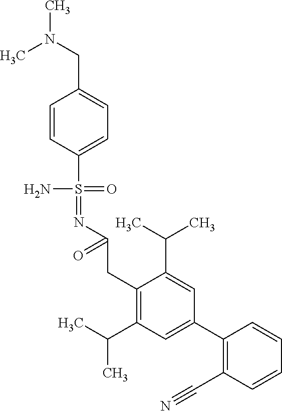 [SMILES: CC(C)c1cc(-c2ccccc2C#N)cc(C(C)C)c1CC(=O)N=S(N)(=O)c1ccc(CN(C)C)cc1] |
| 191 | 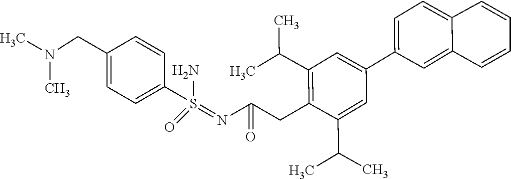 [SMILES: CC(C)c1cc(-c2ccc3ccccc3c2)cc(C(C)C)c1CC(=O)N=S(N)(=O)c1ccc(CN(C)C)cc1] |
| 192 | 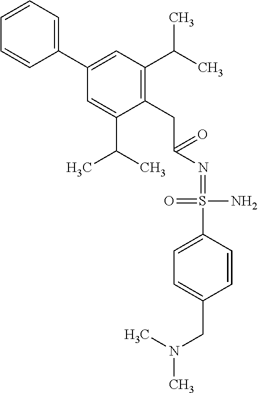 [SMILES: CC(C)c1cc(-c2ccccc2)cc(C(C)C)c1CC(=O)N=S(N)(=O)c1ccc(CN(C)C)cc1] |
| 194 | 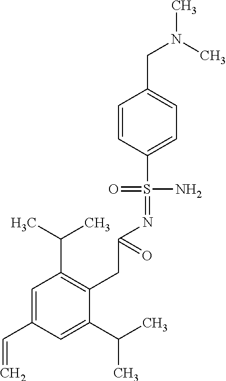 [SMILES: C=Cc1cc(C(C)C)c(CC(=O)N=S(N)(=O)c2ccc(CN(C)C)cc2)c(C(C)C)c1] |
| 195 | 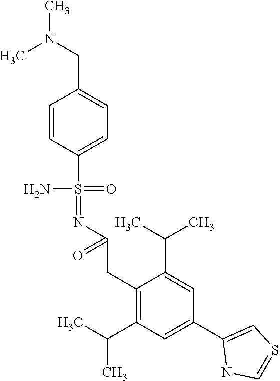 |
| 196 | 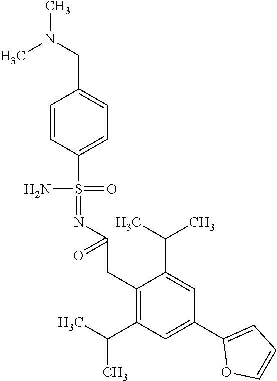 [SMILES: CC(C)c1cc(-c2ccco2)cc(C(C)C)c1CC(=O)N=S(N)(=O)c1ccc(CN(C)C)cc1] |
| 197 | 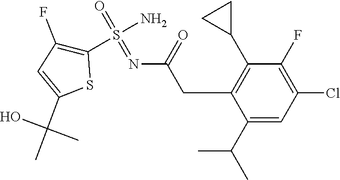 [SMILES: CC(C)c1cc(Cl)c(F)c(C2CC2)c1CC(=O)N=S(N)(=O)c1sc(C(C)(C)O)cc1F] |
| 197a | 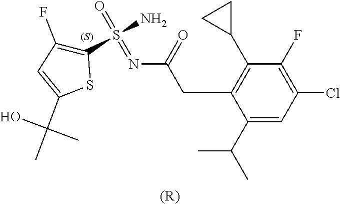 |
| 197b | 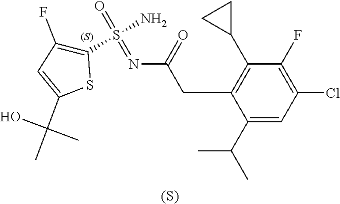 |
| 198 | 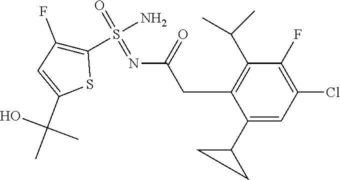 [SMILES: CC(C)c1c(F)c(Cl)cc(C2CC2)c1CC(=O)N=S(N)(=O)c1sc(C(C)(C)O)cc1F] |
| 198a | (R) |
| 198b | (S) |

and pharmaceutically acceptable salts thereof.
and pharmaceutically acceptable salts thereof.

135. A compound selected from the group consisting of the compounds below:

| Cmpd # | Structure |
|---|---|
| 104a | 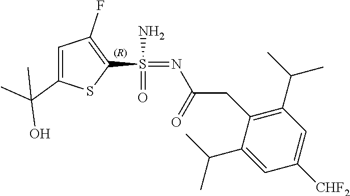 |
| 104b | 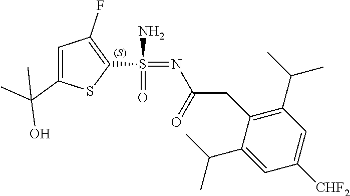 |
| 106a | 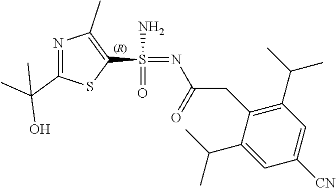 |
| 106b | 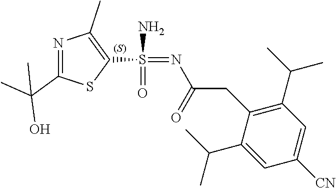 |
| 107a | 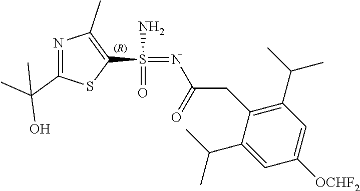 |
| 107b | 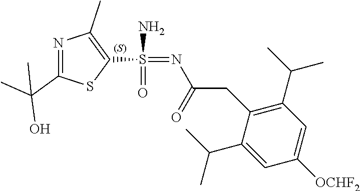 |
| 110a | 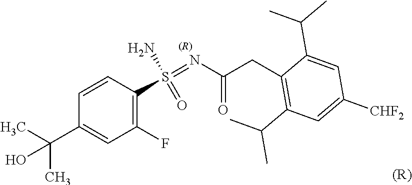 |
| 110b | 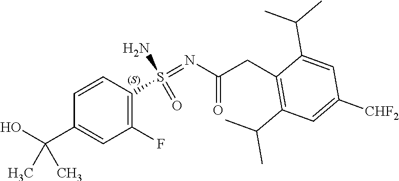 |
| 126a | 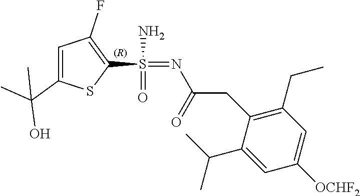 |
| 126b | 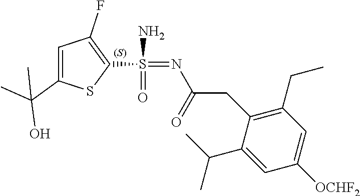 |
| 130a | 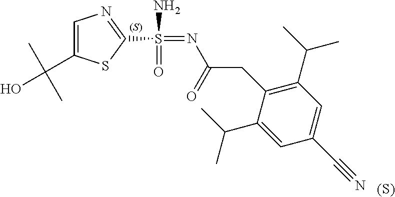 |
| 130b | 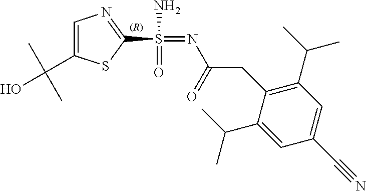 |
| 146 | 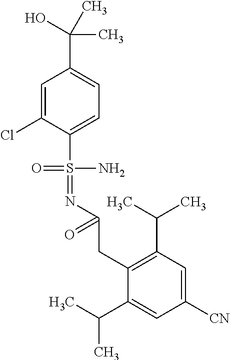 [SMILES: CC(C)c1cc(C#N)cc(C(C)C)c1CC(=O)N=S(N)(=O)c1ccc(C(C)(C)O)cc1Cl] |
| 147 | 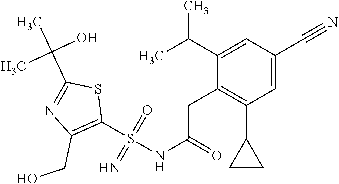 [SMILES: CC(C)c1cc(C#N)cc(C2CC2)c1CC(=O)NS(=N)(=O)c1sc(C(C)(C)O)nc1CO] |
| 148 | 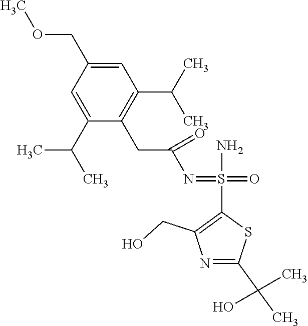 [SMILES: COCc1cc(C(C)C)c(CC(=O)N=S(N)(=O)c2sc(C(C)(C)O)nc2CO)c(C(C)C)c1] |
| 149a | 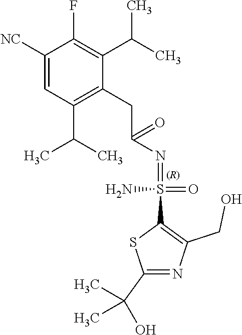 |
| 149b | 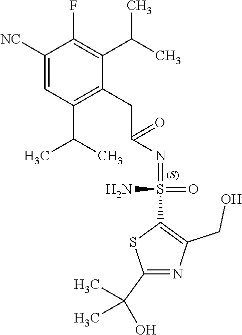 |
| 150 | 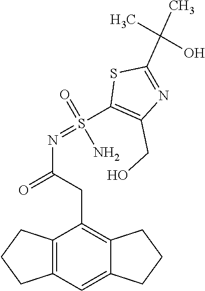 [SMILES: CC(C)(O)c1nc(CO)c(S(N)(=O)=NC(=O)Cc2c3c(cc4c2CCC4)CCC3)s1] |
| 151 | 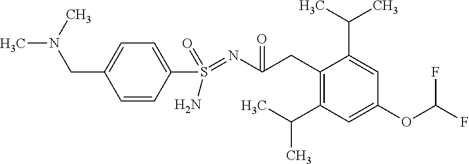 [SMILES: CC(C)c1cc(OC(F)F)cc(C(C)C)c1CC(=O)N=S(N)(=O)c1ccc(CN(C)C)cc1] |
| 152 | 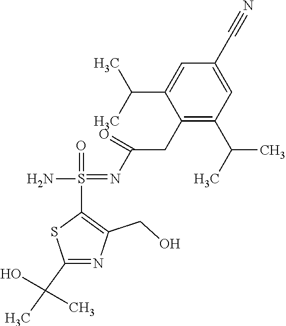 [SMILES: CC(C)c1cc(C#N)cc(C(C)C)c1CC(=O)N=S(N)(=O)c1sc(C(C)(C)O)nc1CO] |
| 152a | 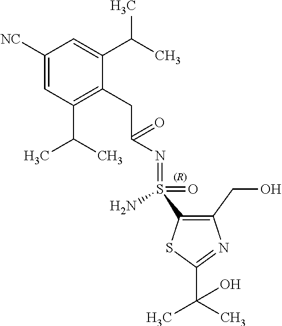 |
| 152b | 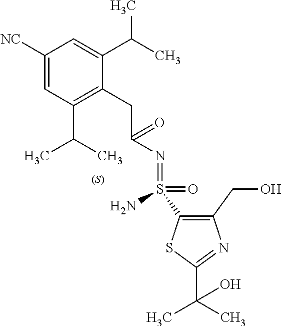 |

and pharmaceutically acceptable salts thereof.

136. A compound selected from the group consisting of the compounds below: 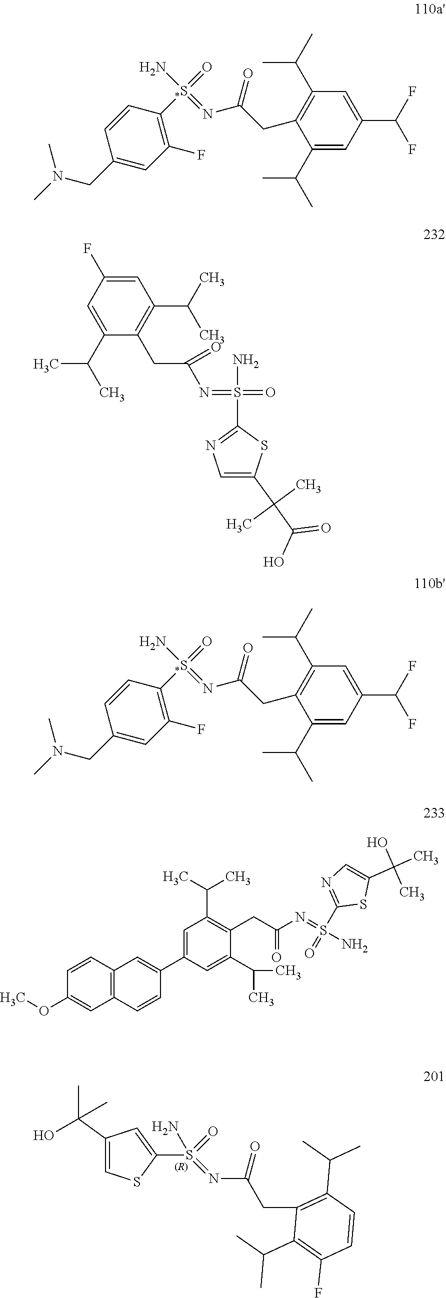 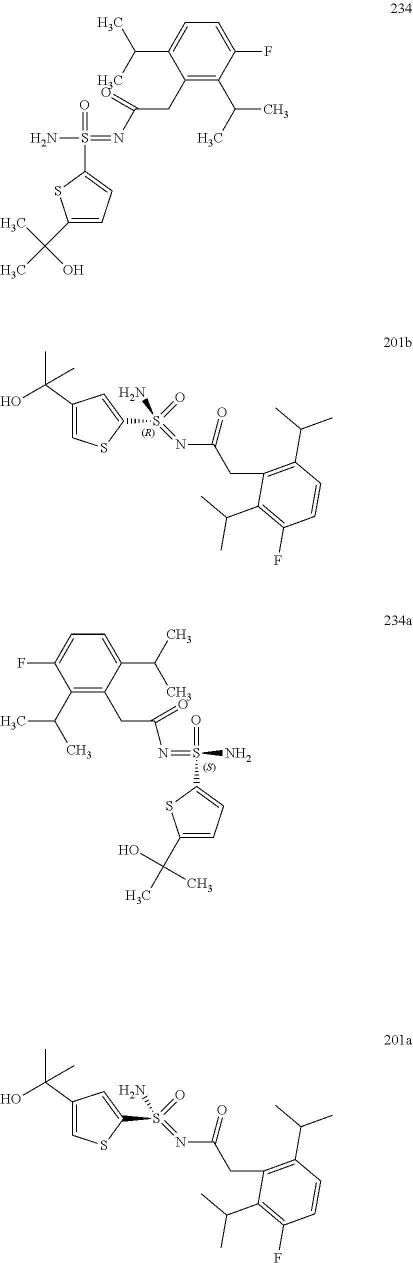 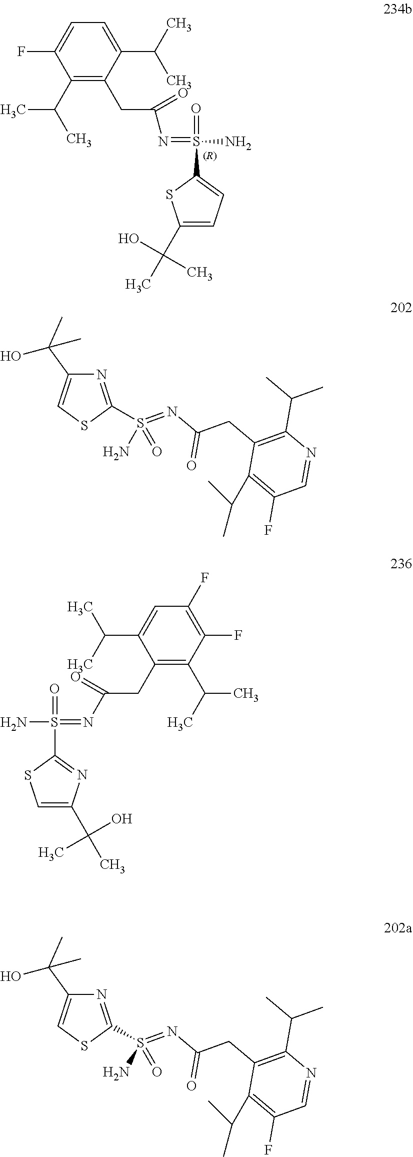 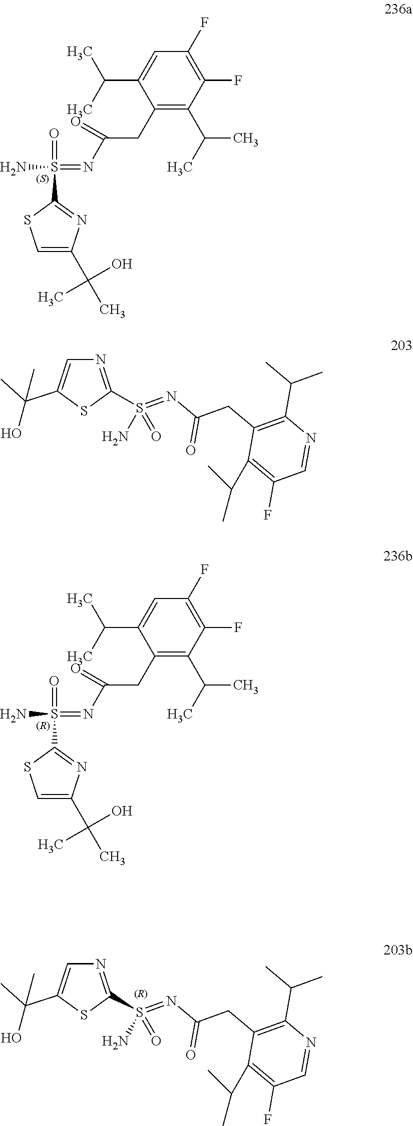 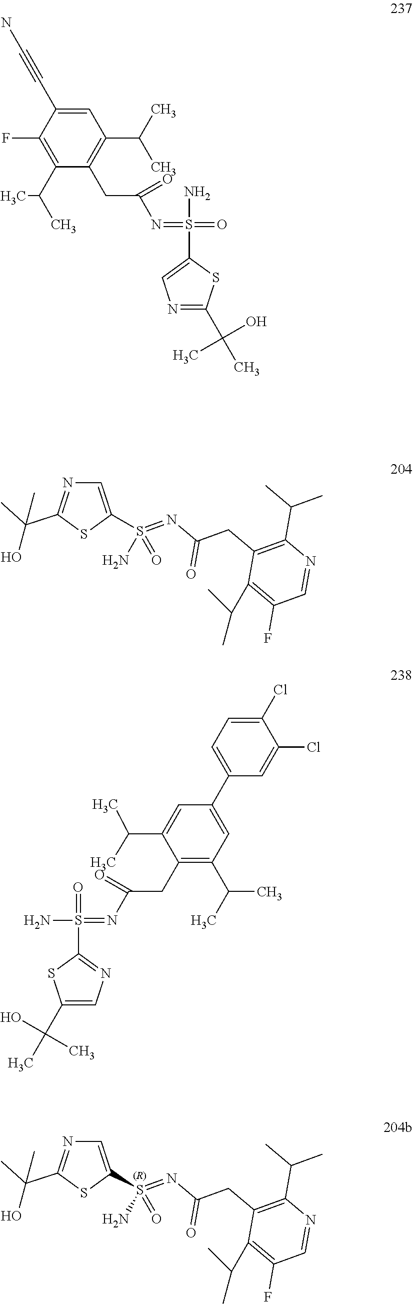 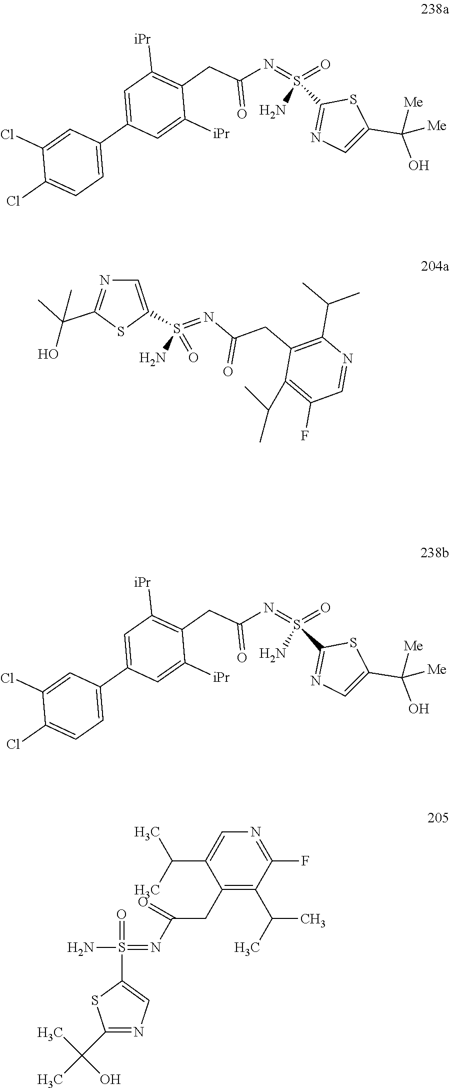 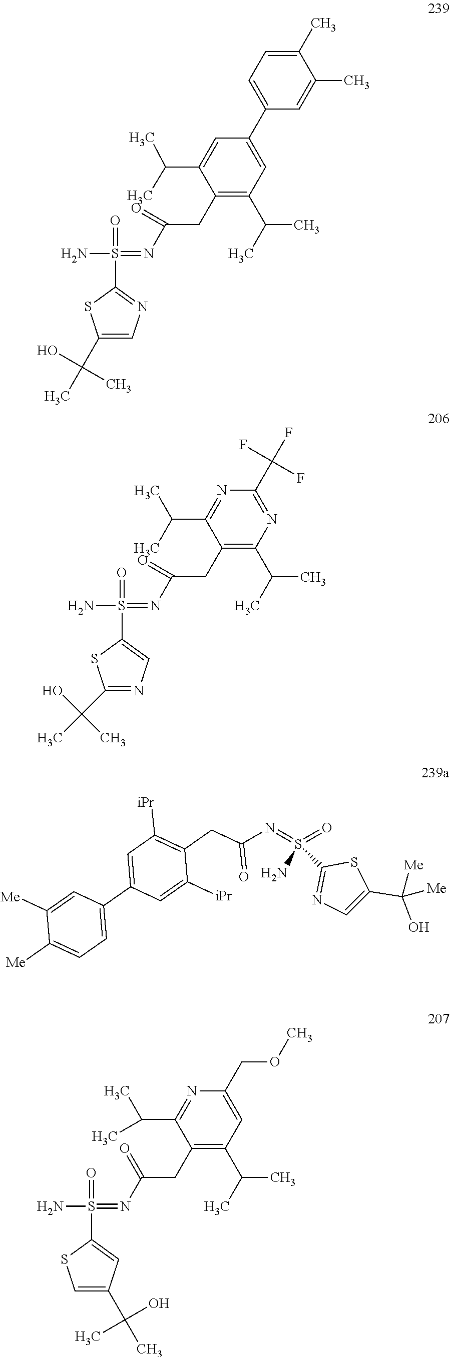 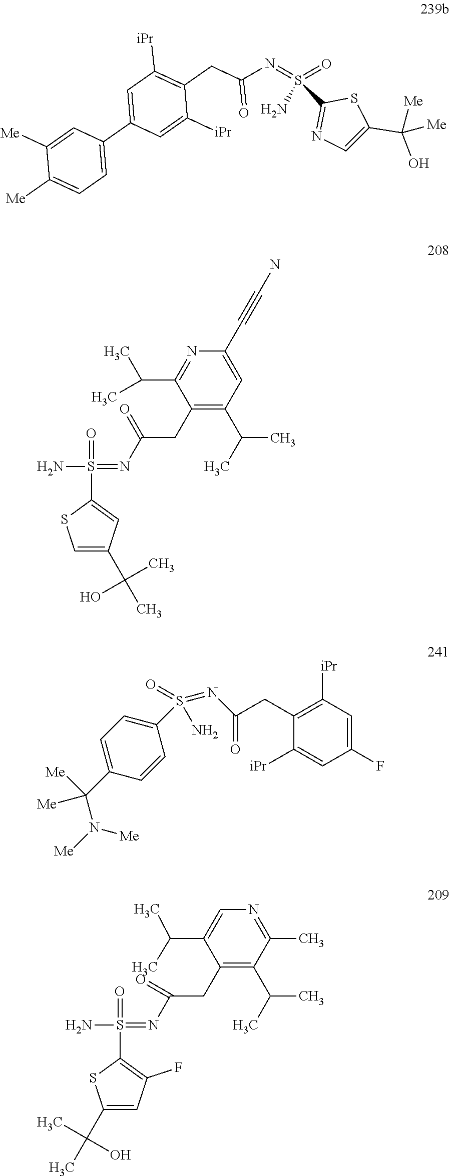 [SMILES: Cc1ccc(-c2cc(C(C)C)c(CC(=O)N=[S@@](N)(=O)c3ncc(C(C)(C)O)s3)c(C(C)C)c2)cc1C ; CC(C)c1cc(C#CN)nc(C(C)C)c1CC(=O)N=S(N)(=O)c1cc(C(C)(C)O)cs1 ; CC(C)c1cc(F)cc(C(C)C)c1CC(=O)N=S(N)(=O)c1ccc(C(C)(C)N(C)C)cc1 ; Cc1ncc(C(C)C)c(CC(=O)N=S(N)(=O)c2sc(C(C)(C)O)cc2F)c1C(C)C] 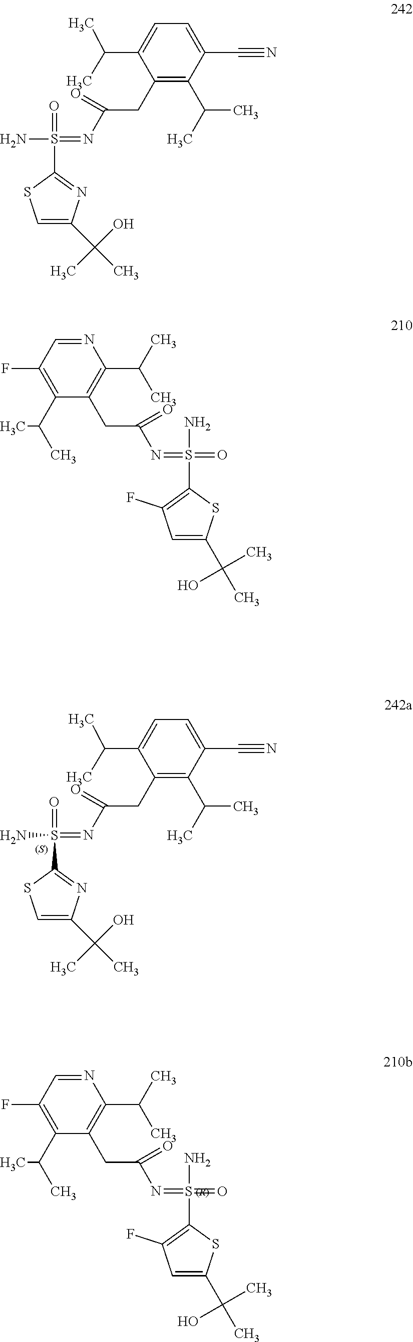 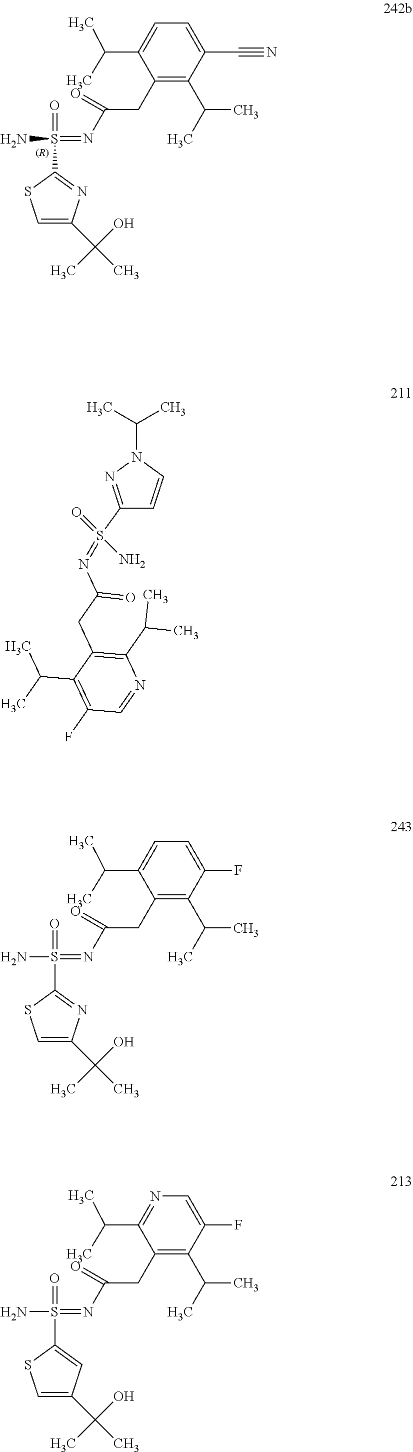 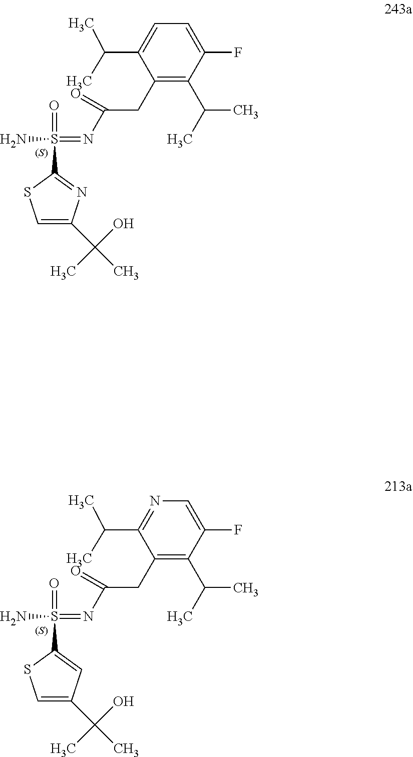 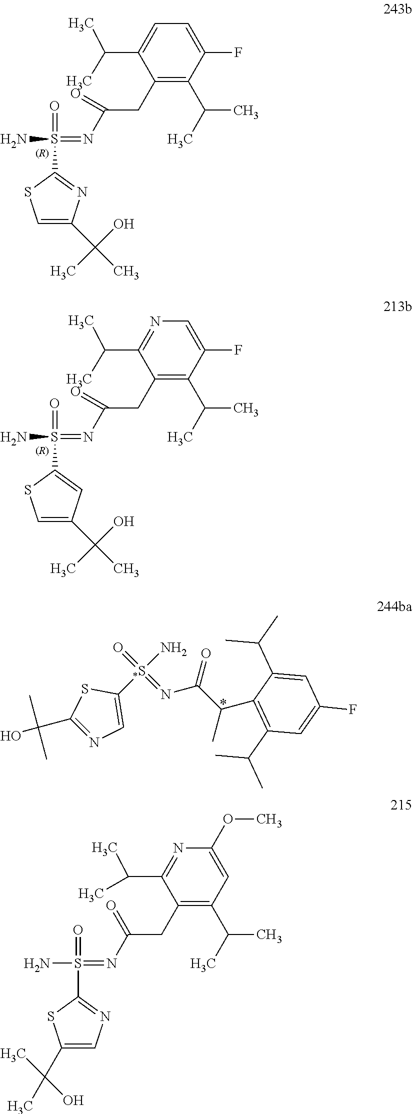 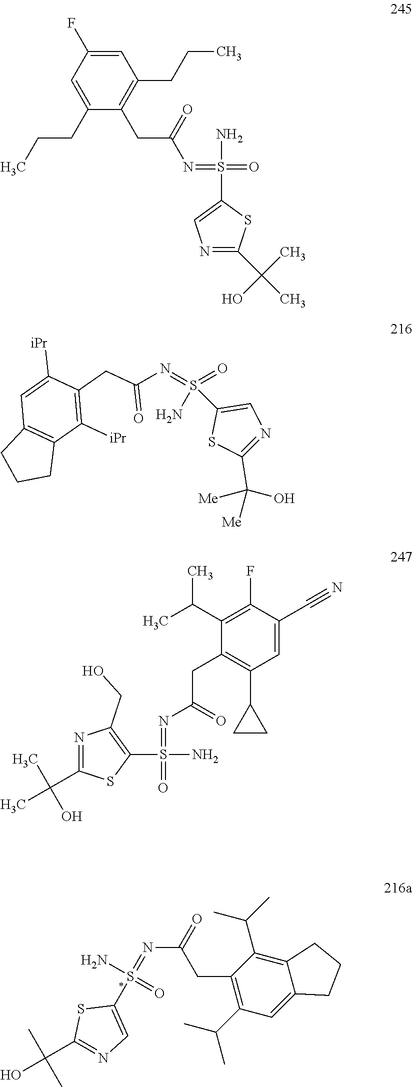 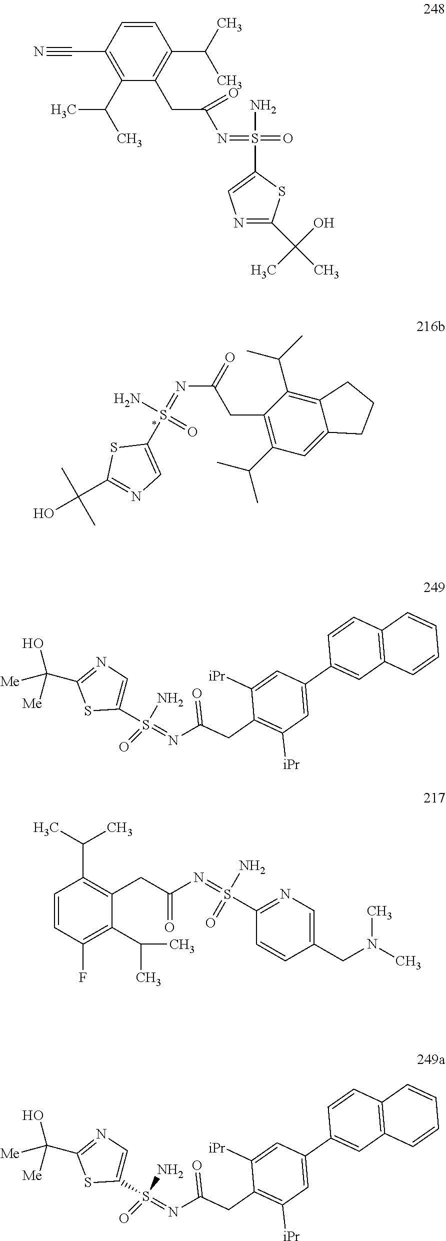 [SMILES: CC(C)c1ccc(C#N)c(C(C)C)c1CC(=O)N=S(N)(=O)c1cnc(C(C)(C)O)s1 ; CC(C)c1cc2c(c(C(C)C)c1CC(=O)N=S(N)(=O)c1cnc(C(C)(C)O)s1)CCC2 ; CC(C)c1cc(-c2ccc3ccccc3c2)cc(C(C)C)c1CC(=O)N=S(N)(=O)c1cnc(C(C)(C)O)s1 ; CC(C)c1ccc(F)c(C(C)C)c1CC(=O)N=S(N)(=O)c1ccc(CN(C)C)cn1 ; CC(C)c1cc(-c2ccc3ccccc3c2)cc(C(C)C)c1CC(=O)N=[S@](N)(=O)c1cnc(C(C)(C)O)s1] 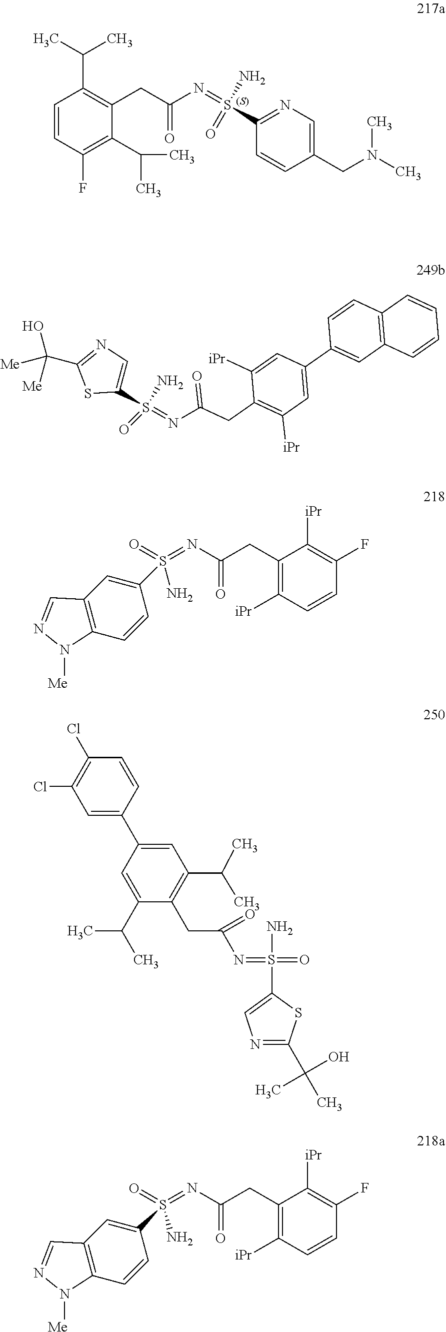 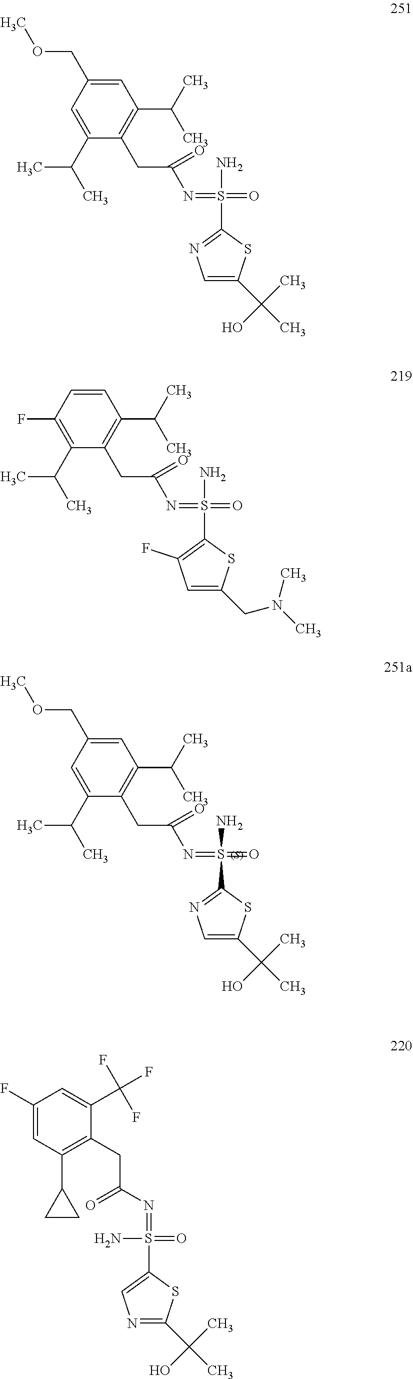 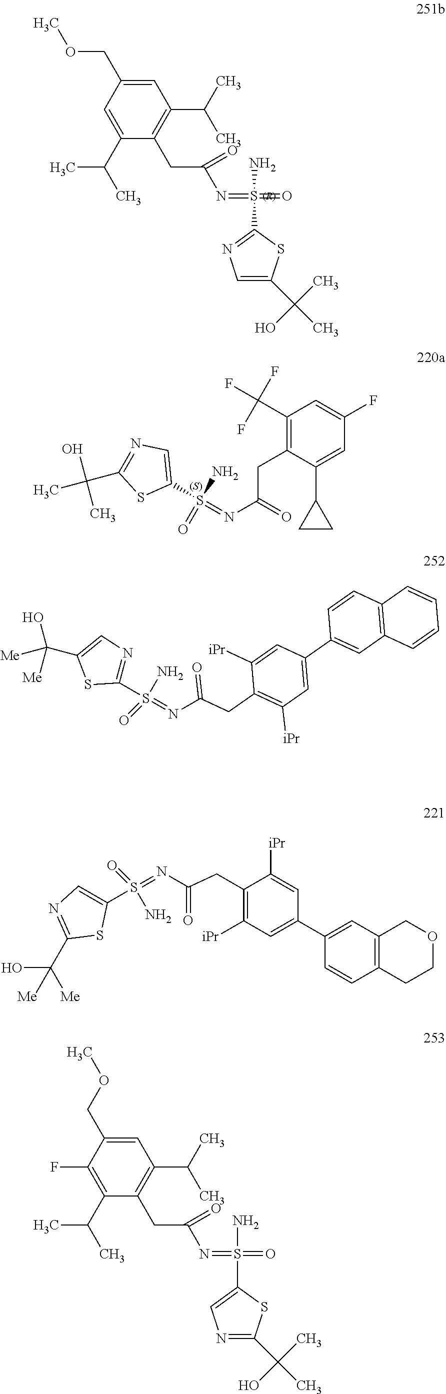 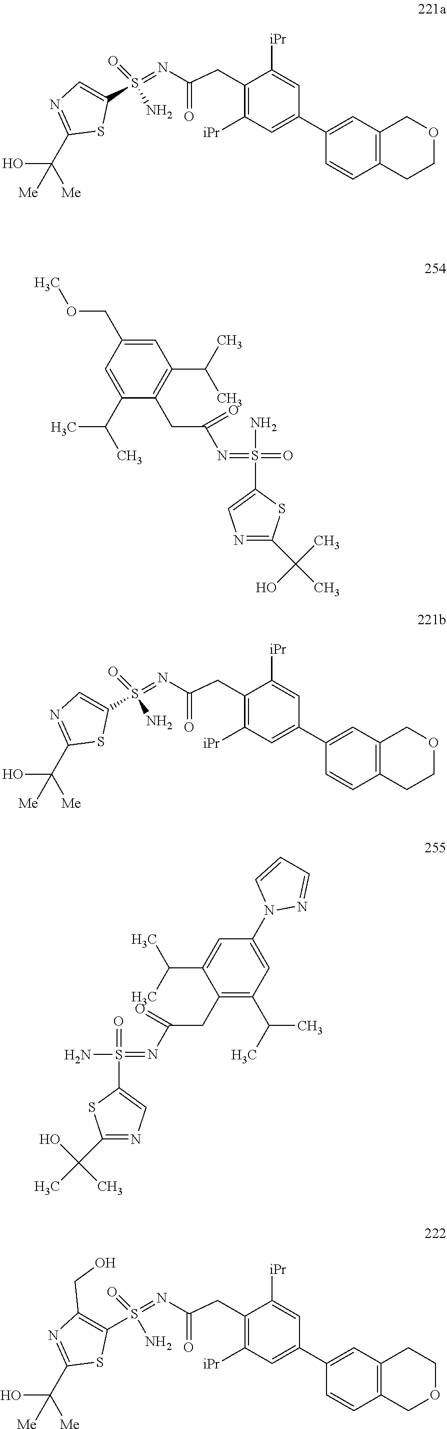 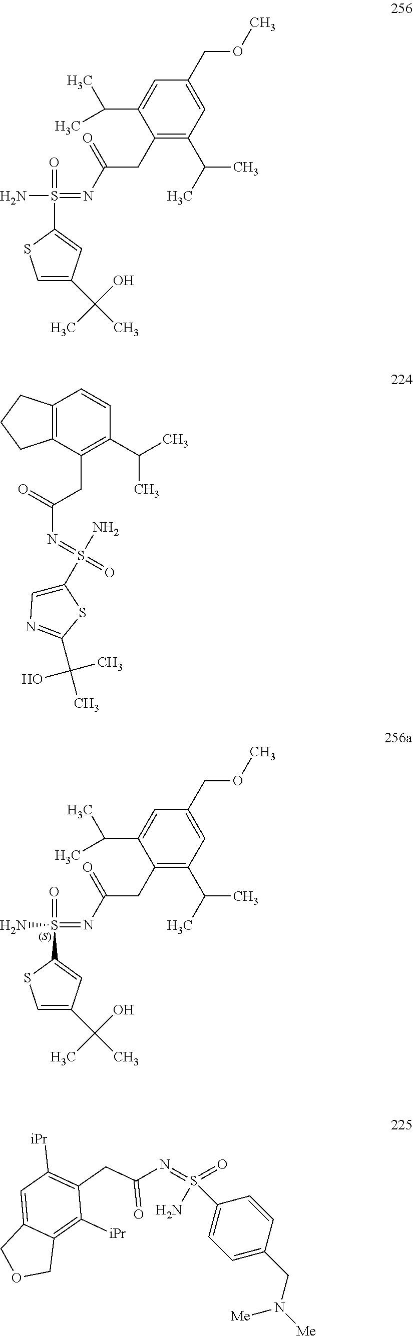 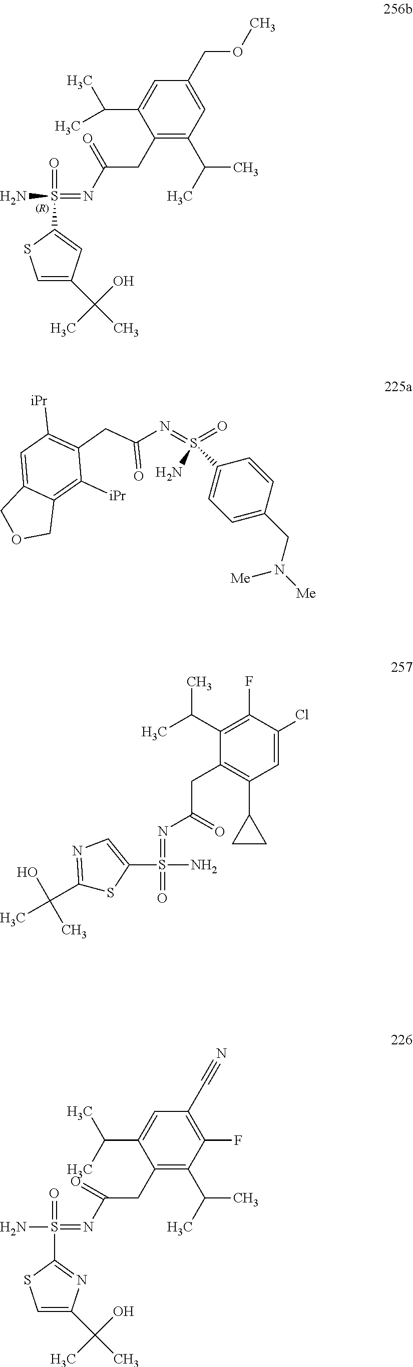 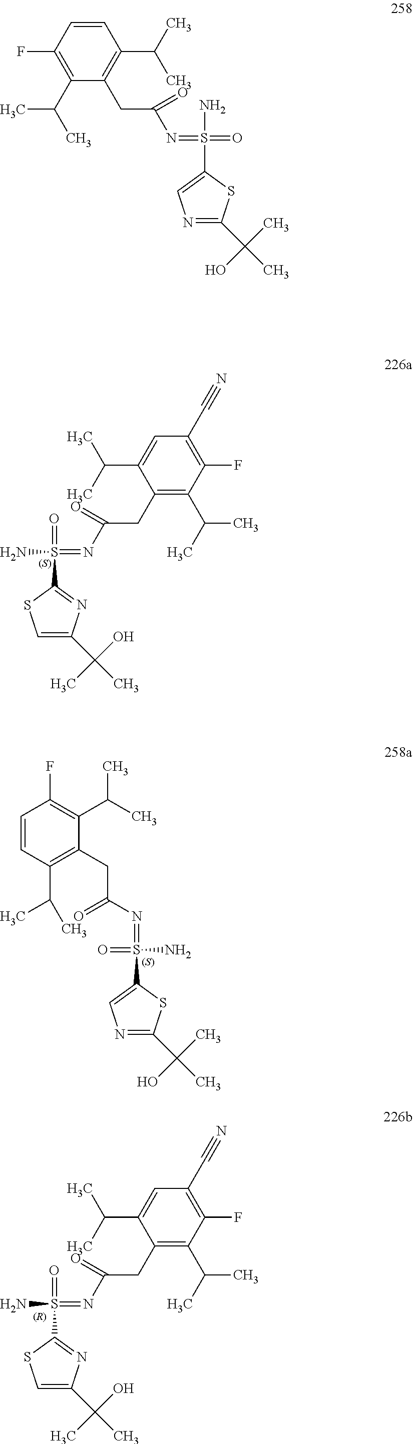 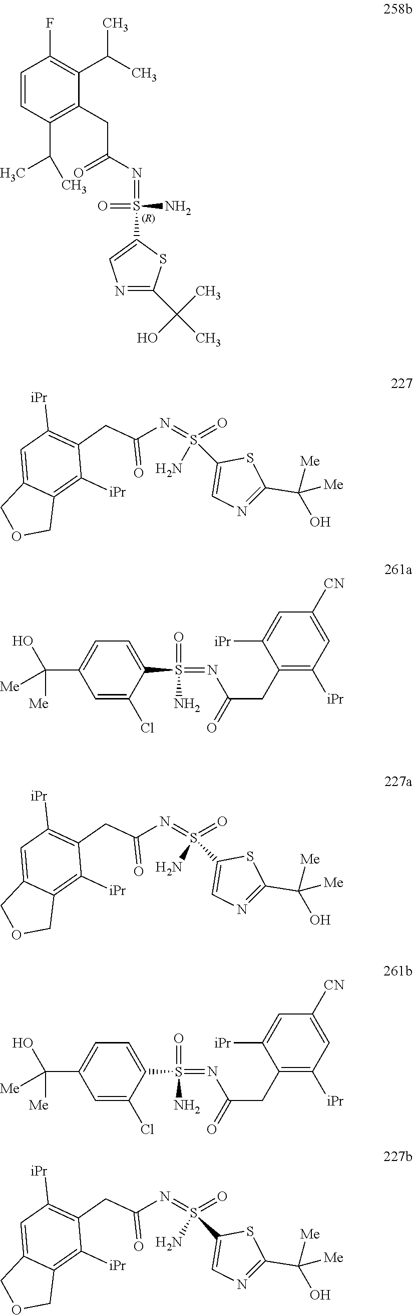 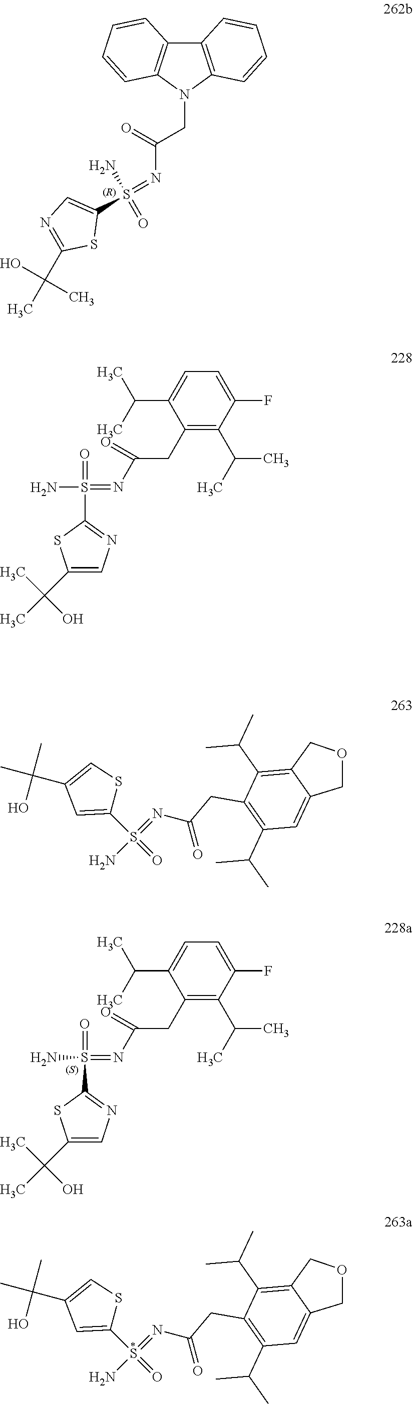 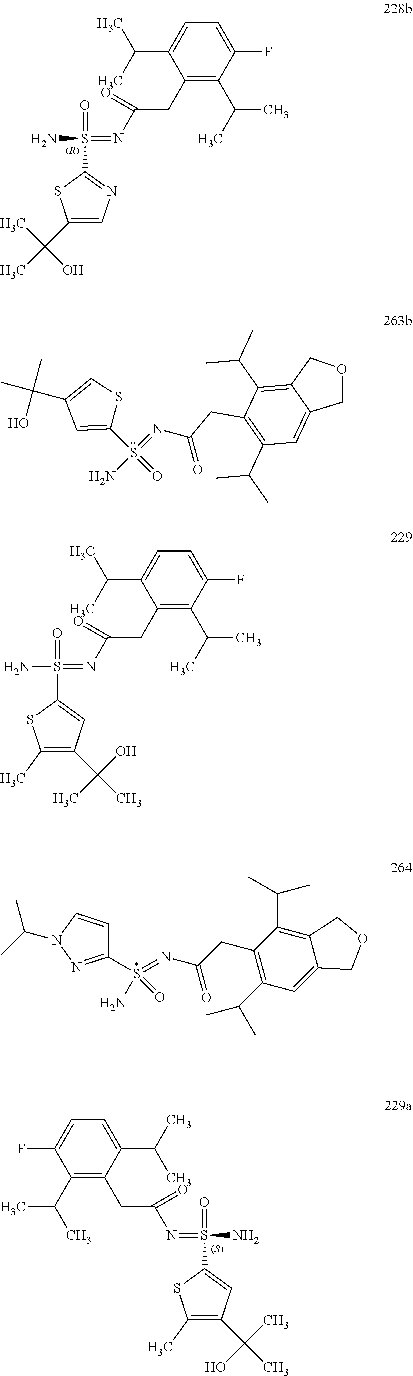 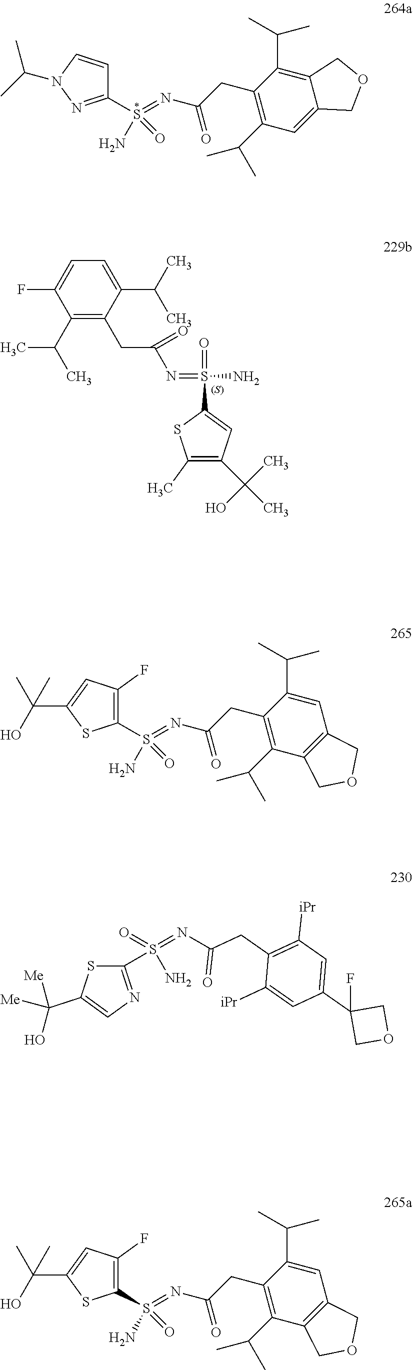 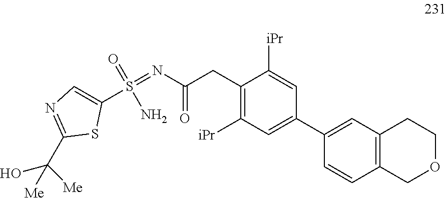 [SMILES: CC(C)c1cc(-c2ccc3c(c2)CCOC3)cc(C(C)C)c1CC(=O)N=S(N)(=O)c1cnc(C(C)(C)O)s1]
and a pharmaceutically acceptable salt thereof.

137. The compound of claim 1, wherein the compound is selected from Table 1-4.

138. The compound ofany one of claims 1-2, wherein the compound is selected from Table 1-5.

139. The compound of any one of claims 1-2, wherein the compound is selected from Table 1-6.

140. The compound of any one of claims 1-2, wherein the compound is selected from Table 1-7.

141. The compound of any one of claims 1-140, wherein the sulfur in the moiety S(═O)(NHR³)═N— has (S) stereochemistry.

142. The compound of any one of claims 1-140, wherein the sulfur in the moiety S(═O)(NHR³)═N— has (R) stereochemistry.

143. A pharmaceutical composition comprising a compound or salt as claimed in any one of claims 1-142 and one or more pharmaceutically acceptable excipients.

144. A method for modulating NLRP3 activity, the method comprising contacting NLRP3 with an effective amount of a compound as claimed in any one of claims 1-142 or a pharmaceutical composition as claimed in claim 143.

145. The method of claim 144, wherein the modulating comprises antagonizing NLRP3.

146. The method of any one of claim 144 or 145, which is carried out in vitro.

147. The method of any one of claims 144-146, wherein the method comprises contacting a sample comprising one or more cells comprising NLRP3 with the compound.

148. The method of any one of claims 144-146, which is carried out in vivo.

149. The method of claim 148, wherein the method comprises administering the compound to a subject having a disease in which NLRP3 signaling contributes to the pathology and/or symptoms and/or progression of the disease.

150. The method of claim 149, wherein the subject is a human.

151. A method of treating a disease, disorder or condition that is a metabolic disorder, comprising administering to a subject in need of such treatment an effective amount of a compound as claimed in any one of claims 1-142 or a pharmaceutical composition as claimed in claim 143.

152. The method of claim 151, wherein the metabolic disorder is Type 2 diabetes, atherosclerosis, obesity or gout.

153. A method of treating a disease, disorder or condition that is a disease of the central nervous system, comprising administering to a subject in need of such treatment an effective amount of a compound as claimed in any one of claims 1-142 or a pharmaceutical composition as claimed in claim 143.

154. The method of claim 153, wherein the disease of the central nervous system is Alzheimer's disease, multiple sclerosis, Amyotrophic Lateral Sclerosis or Parkinson's disease.

155. A method of treating a disease, disorder or condition that is lung disease, comprising administering to a subject in need of such treatment an effective amount of a compound as claimed in any one of claims 1-142 or a pharmaceutical composition as claimed in claim 143.

156. The method of claim 155, wherein the lung disease is asthma, COPD or pulmonary idiopathic fibrosis.

157. A method of treating a disease, disorder or condition that is liver disease, comprising administering to a subject in need of such treatment an effective amount of a compound as claimed in any one of claims 1-142 or a pharmaceutical composition as claimed in claim 143.

158. The method of claim 157, wherein the liver disease is NASH syndrome, viral hepatitis or cirrhosis.

159. A method of treating a disease, disorder or condition that is pancreatic disease, comprising administering to a subject in need of such treatment an effective amount of a compound as claimed in any one of claims 1-142 or a pharmaceutical composition as claimed in claim 143.

160. The method of claim 159, wherein the pancreatic disease is acute pancreatitis or chronic pancreatitis.

161. A method of treating a disease, disorder or condition that is kidney disease, comprising administering to a subject in need of such treatment an effective amount of a compound as claimed in any one of claims 1-142 or a pharmaceutical composition as claimed in claim 143.

162. The method of claim 161, wherein the kidney disease is acute kidney injury or chronic kidney injury.

163. A method of treating a disease, disorder or condition that is intestinal disease, comprising administering to a subject in need of such treatment an effective amount of a compound as claimed in any one of claims 1-142 or a pharmaceutical composition as claimed in claim 143.

164. The method of claim 163, wherein the intestinal disease is Crohn's disease or Ulcerative Colitis.

165. A method of treating a disease, disorder or condition that is skin disease, comprising administering to a subject in need of such treatment an effective amount of a compound as claimed in any one of claims 1-142 or a pharmaceutical composition as claimed in claim 143.

166. The method of claim 165, wherein the skin disease is psoriasis.

167. A method of treating a disease, disorder or condition that is musculoskeletal disease, comprising administering to a subject in need of such treatment an effective amount of a compound as claimed in any one of claims 1-142 or a pharmaceutical composition as claimed in claim 143.

168. The method of claim 167, wherein the musculoskeletal disease is scleroderma.

169. A method of treating a disease, disorder or condition that is a vessel disorder, comprising administering to a subject in need of such treatment an effective amount of a compound as claimed in any one of claims 1-142 or a pharmaceutical composition as claimed in claim 143.

170. The method of claim 169, wherein the vessel disorder is giant cell arteritis.

171. A method of treating a disease, disorder or condition that is a disorder of the bones, comprising administering to a subject in need of such treatment an effective amount of a compound as claimed in any one of claims 1-142 or a pharmaceutical composition as claimed in claim 143.

172. The method of claim 171, wherein the disorder of the bones is osteoarthritis, osteoporosis or osteopetrosis disorders.

173. A method of treating a disease, disorder or condition that is eye disease, comprising administering to a subject in need of such treatment an effective amount of a compound as claimed in any one of claims 1-142 or a pharmaceutical composition as claimed in claim 143.

174. The method of claim 173, wherein the eye disease is glaucoma or macular degeneration.

175. A method of treating a disease, disorder or condition that is a disease caused by viral infection, comprising administering to a subject in need of such treatment an effective amount of a compound as claimed in any one of claims 1-142 or a pharmaceutical composition as claimed in claim 143.

176. The method of claim 175, wherein the diseases caused by viral infection is HIV or AIDS.

177. A method of treating a disease, disorder or condition that is an autoimmune disease, comprising administering to a subject in need of such treatment an effective amount of a compound as claimed in any one of claims 1-142 or a pharmaceutical composition as claimed in claim 143.

178. The method of claim 177, wherein the autoimmune disease is Rheumatoid Arthritis, Systemic Lupus Erythematosus, Autoimmune Thyroiditis.

179. A method of treating a disease, disorder or condition that is cancer or aging, comprising administering to a subject in need of such treatment an effective amount of a compound as claimed in any one of claims 1-142 or a pharmaceutical composition as claimed in claim 143.

180. A method of treating a disease, disorder or condition that is a cancer selected from: myelodysplastic syndromes (MDS); non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's cell histiocytosis (LCH); multiple myeloma; promyelocytic leukemia; acute myeloid leukemia (AML) chronic myeloid leukemia (CML); gastric cancer; and lung cancer metastasis, comprising administering to a subject in need of such treatment an effective amount of a compound as claimed in any one of claims 1-142 or a pharmaceutical composition as claimed in claim 143.

181. The method of claim 180, wherein the cancer is MDS.

182. The method of claim 180, wherein the cancer is non-small lung cancer.

183. The method of claim 180, wherein the cancer is acute lymphoblastic leukemia.

184. The method of claim 180, wherein the cancer is LCH.

185. The method of claim 180, wherein the cancer is multiple myeloma.

186. The method of claim 180, wherein the cancer is promyelocytic leukemia.

187. The method of claim 180, wherein the cancer is acute myeloid leukemia (AML).

188. The method of claim 180, wherein the cancer is chronic myeloid leukemia (CML).

189. The method of claim 180, wherein the cancer is gastric cancer.

190. The method of claim 180, wherein the cancer is lung cancer metastasis.

191. The method of any one of claims 149-190, further comprising administering a therapeutically effective amount of an anti-TNFα agent to the subject.

192. The method of claim 191, wherein the NLRP3 antagonist is administered to the subject prior to administration of the anti-TNFα agent to the subject.

193. The method of claim 191, wherein the anti-TNFα agent is administered to the subject prior to the administration of the NLRP3 antagonist to the subject.

194. The method of claim 191, wherein the NLRP3 antagonist and the anti-TNFα agent are administered to the subject at substantially the same time.

195. The method of claim 191, wherein the NLRP3 antagonist and the anti-TNFα agent are formulated together in a single dosage form.

196. A compound of any one of claims 1-143, or a method of any one of claims 144-195, wherein the compound of formula AA or the NLRP3 antagonist is not: 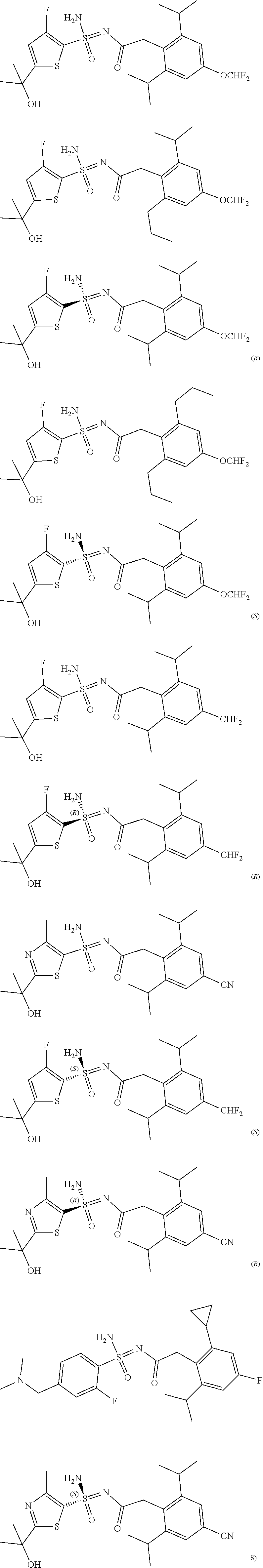 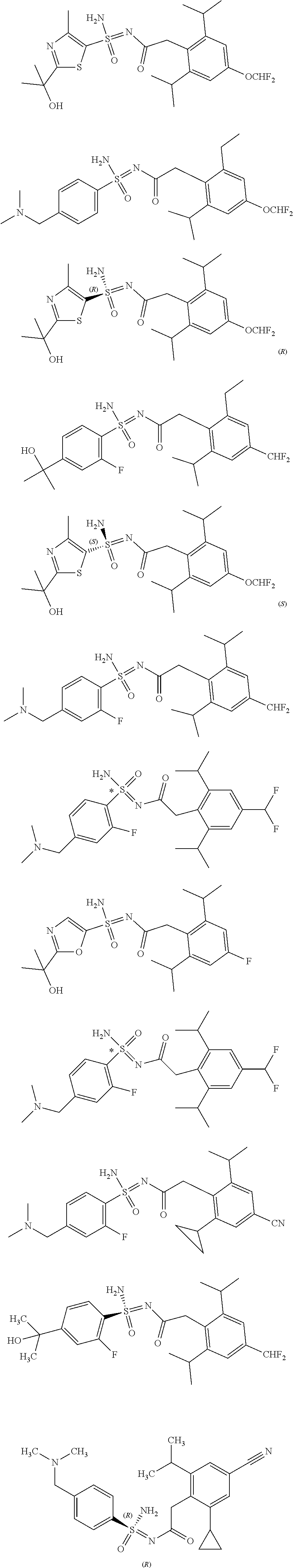 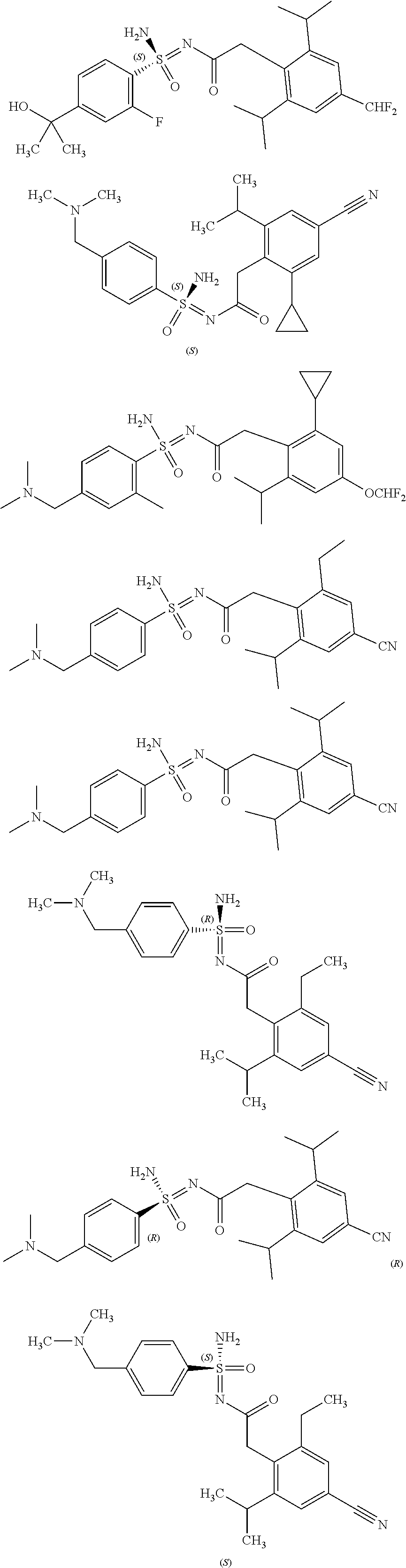 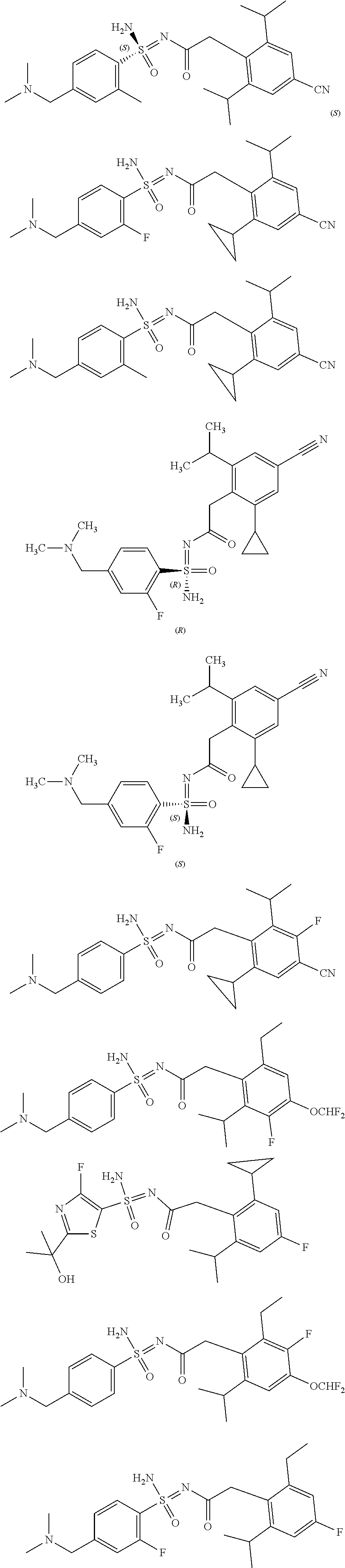 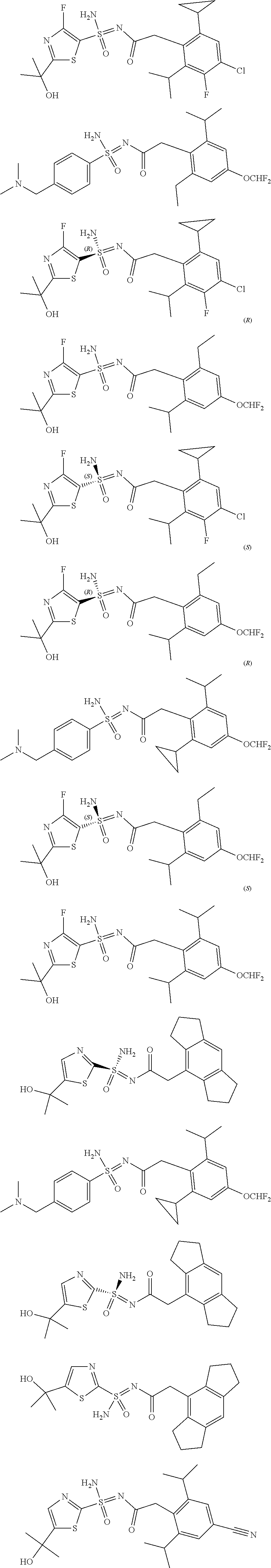 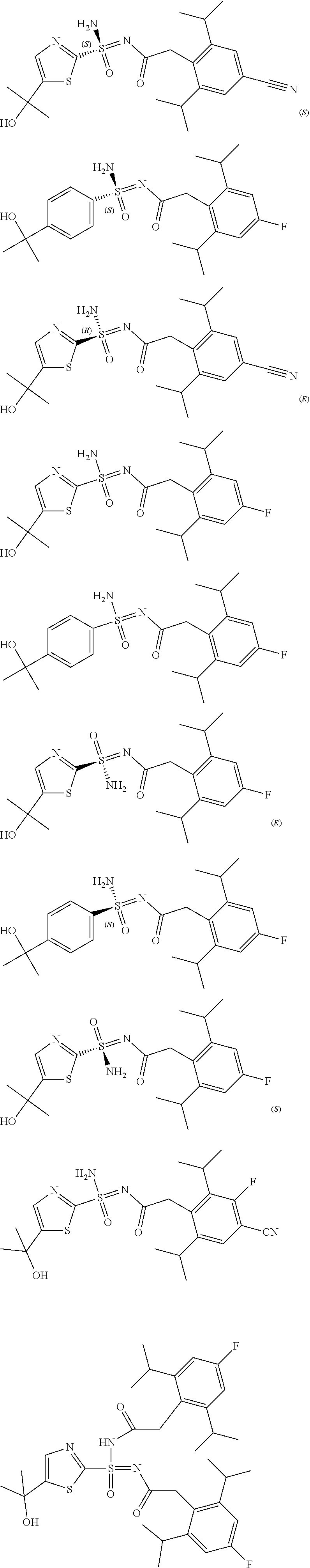 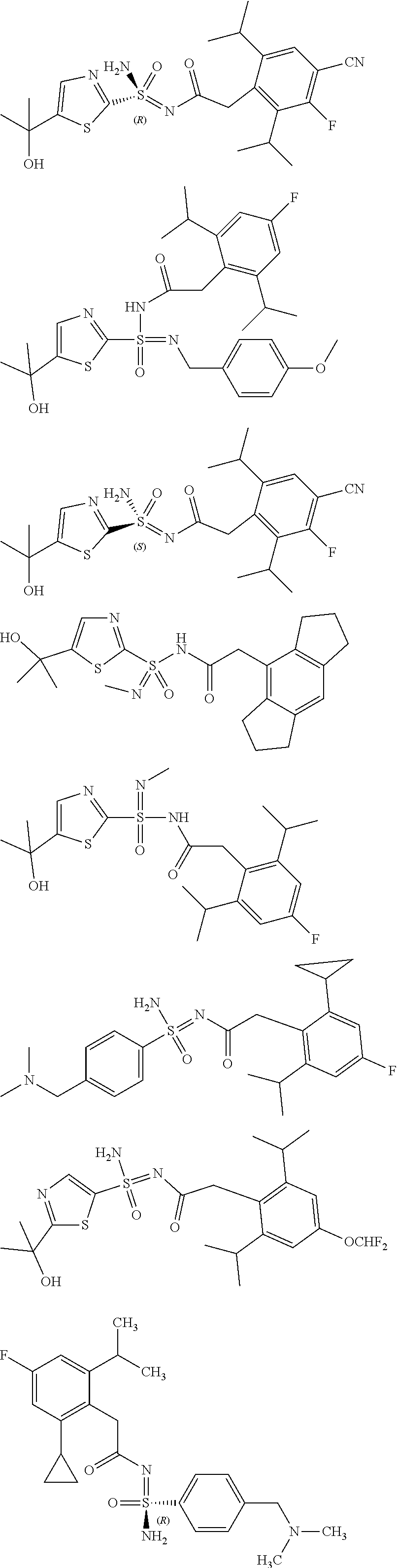 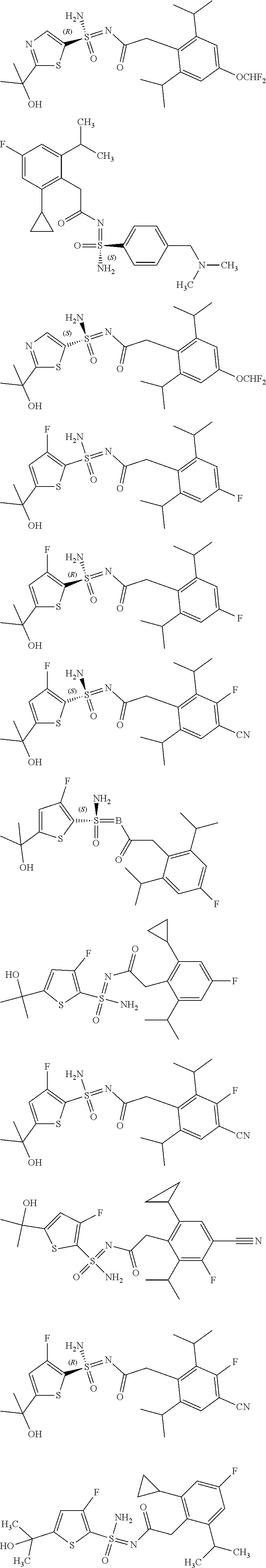 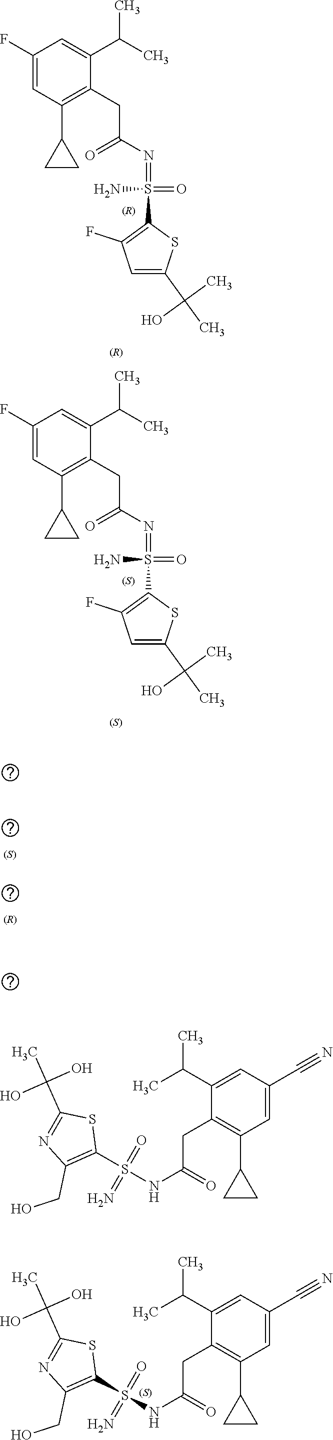 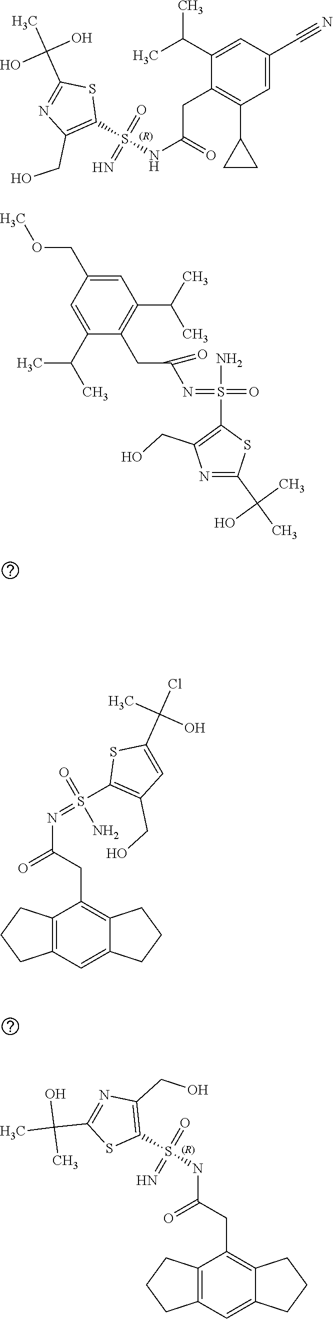 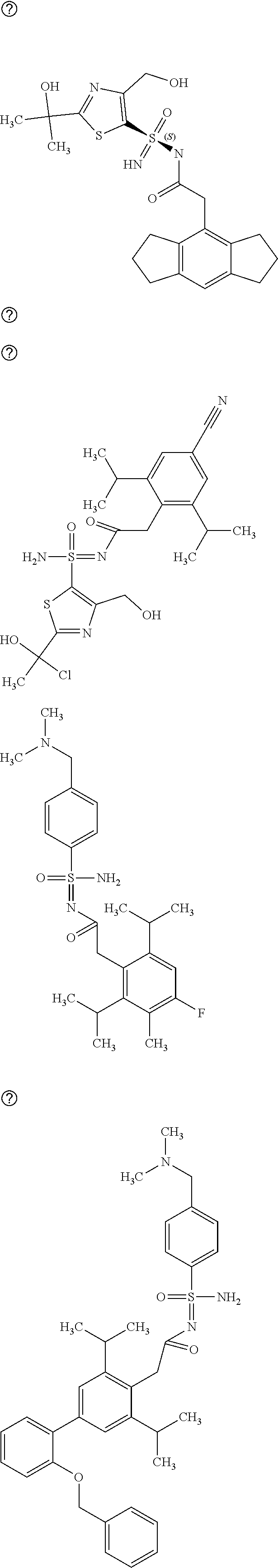 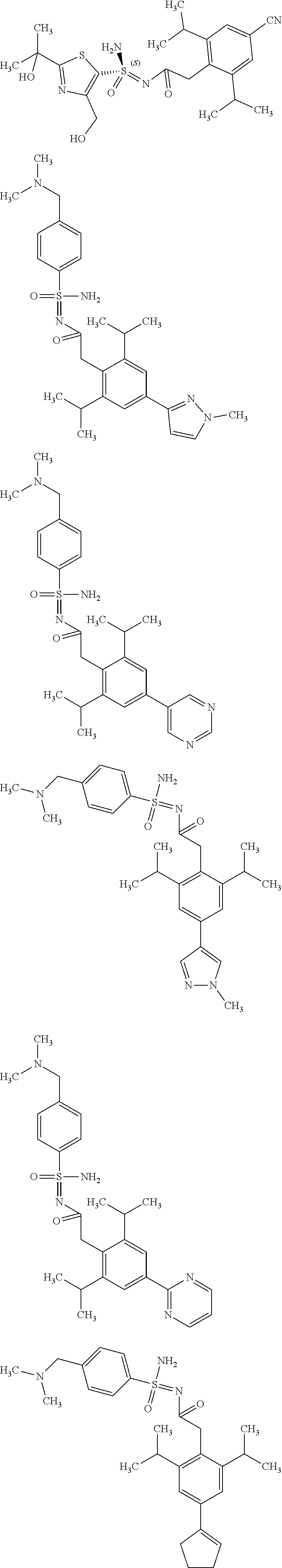 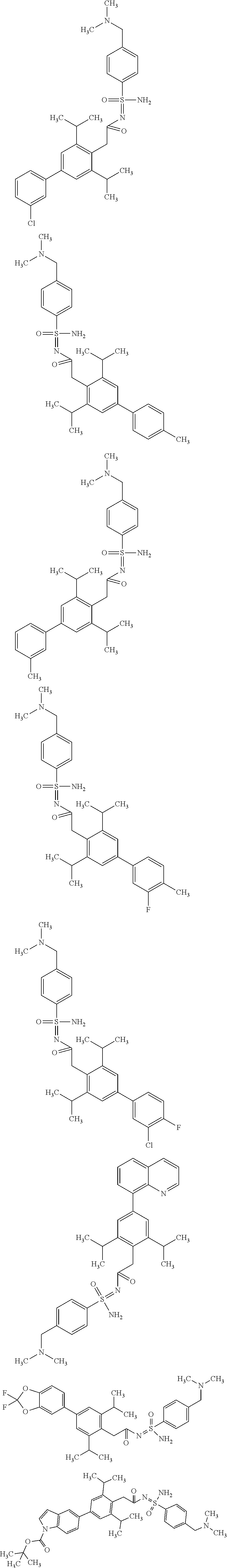 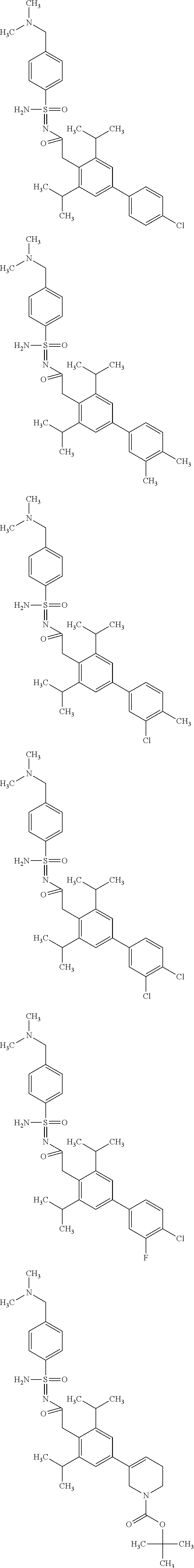 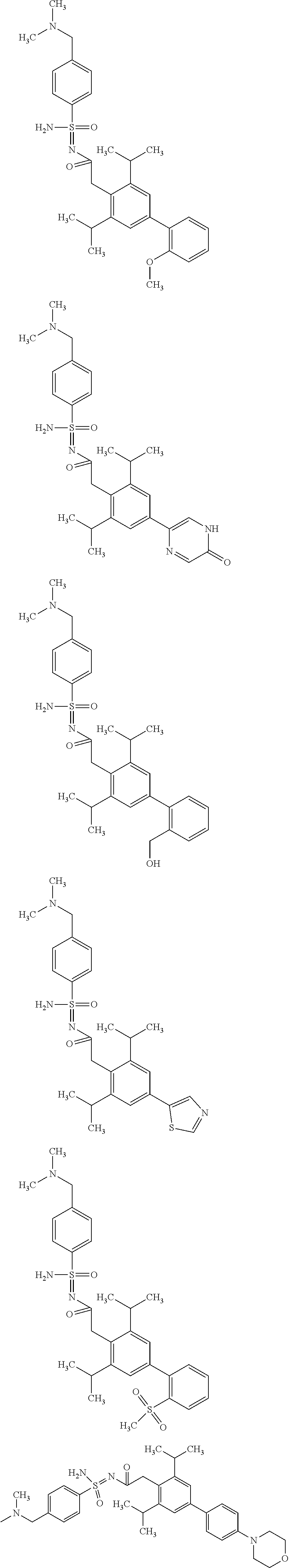 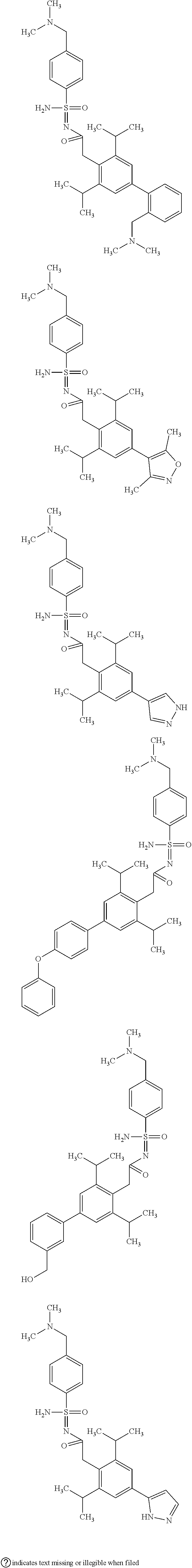 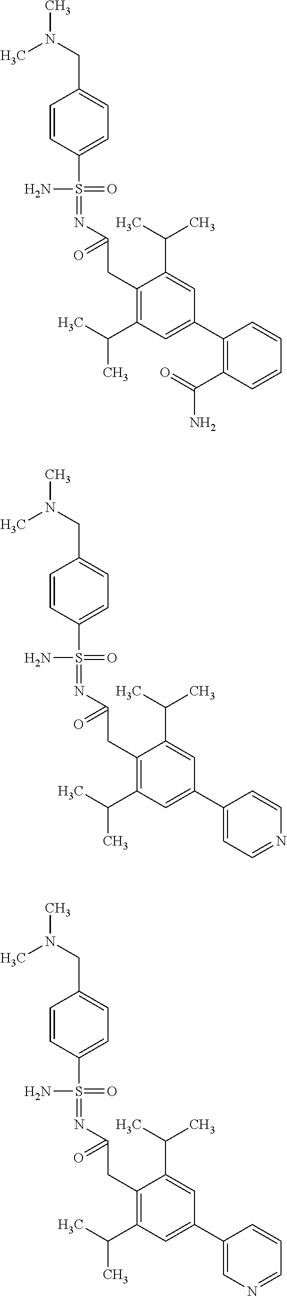 [SMILES: CC(C)c1cc(-c2ccccc2C(N)=O)cc(C(C)C)c1CC(=O)N=S(N)(=O)c1ccc(CN(C)C)cc1 ; CC(C)c1cc(-c2ccncc2)cc(C(C)C)c1CC(=O)N=S(N)(=O)c1ccc(CN(C)C)cc1 ; CC(C)c1cc(-c2cccnc2)cc(C(C)C)c1CC(=O)N=S(N)(=O)c1ccc(CN(C)C)cc1] 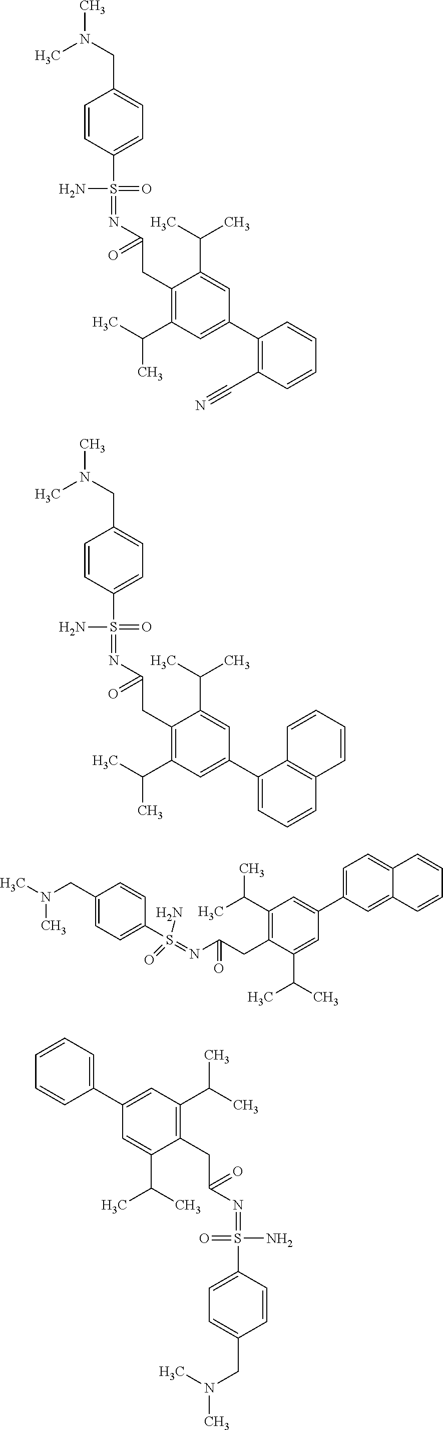 [SMILES: CC(C)c1cc(-c2ccccc2C#N)cc(C(C)C)c1CC(=O)N=S(N)(=O)c1ccc(CN(C)C)cc1 ; CC(C)c1cc(-c2cccc3ccccc23)cc(C(C)C)c1CC(=O)N=S(N)(=O)c1ccc(CN(C)C)cc1 ; CC(C)c1cc(-c2ccc3ccccc3c2)cc(C(C)C)c1CC(=O)N=S(N)(=O)c1ccc(CN(C)C)cc1 ; CC(C)c1cc(-c2ccccc2)cc(C(C)C)c1CC(=O)N=S(N)(=O)c1ccc(CN(C)C)cc1] 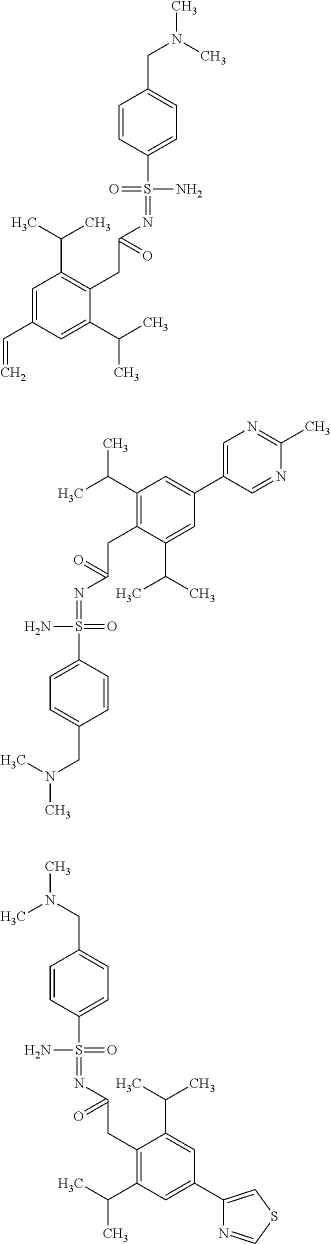 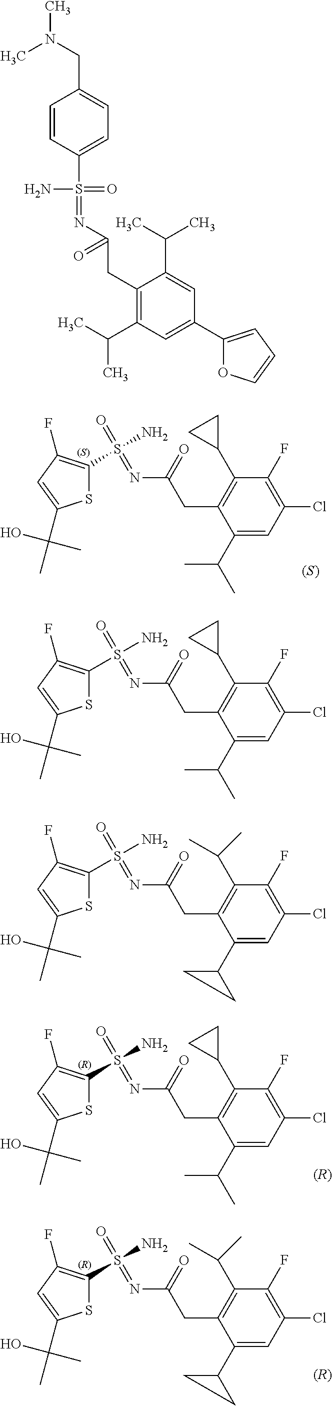 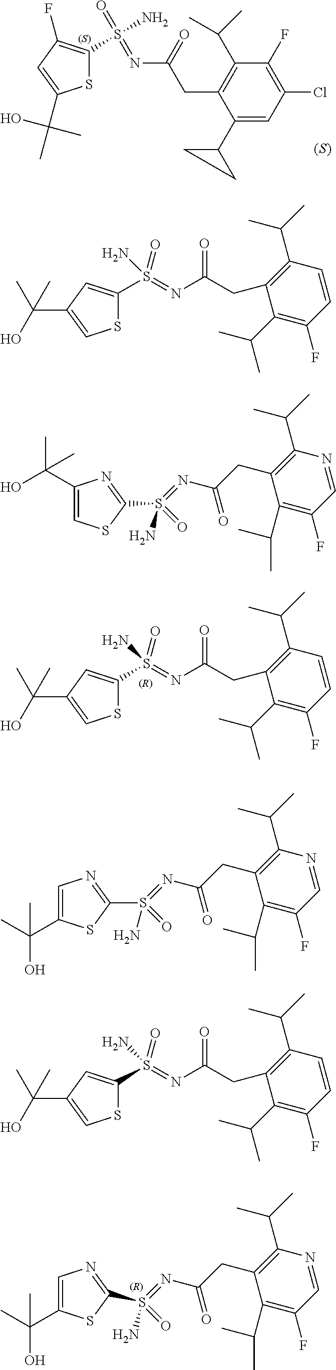 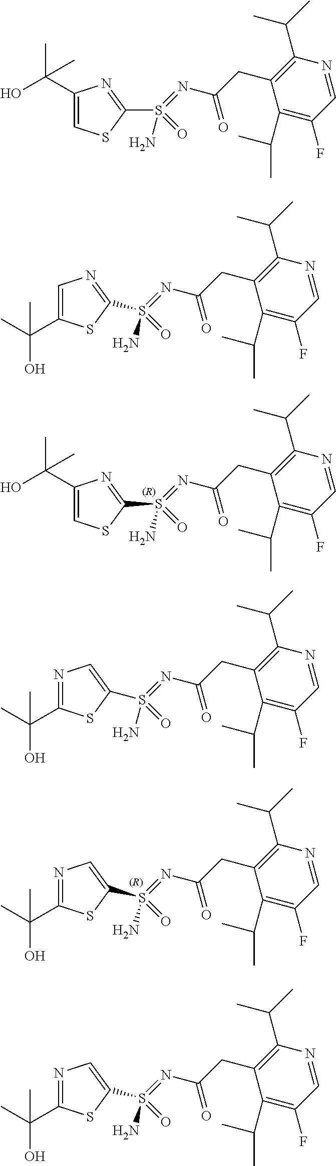 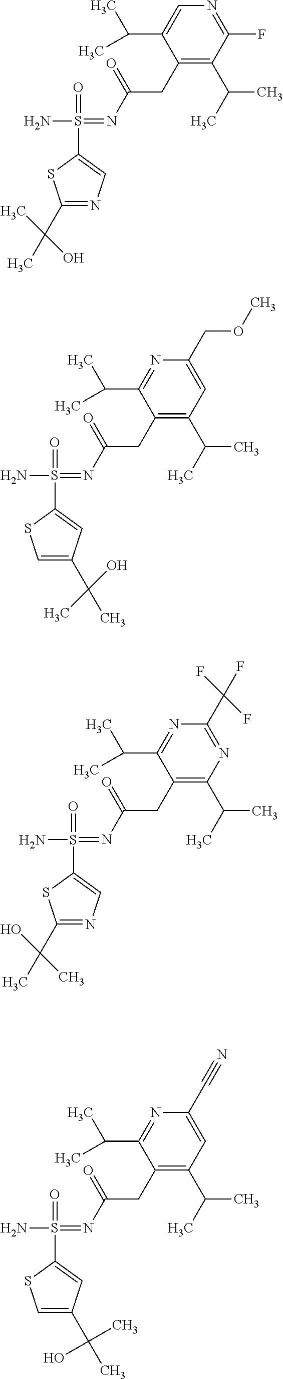 [SMILES: CC(C)c1cnc(F)c(C(C)C)c1CC(=O)N=S(N)(=O)c1cnc(C(C)(C)O)s1 ; COCc1cc(C(C)C)c(CC(=O)N=S(N)(=O)c2cc(C(C)(C)O)cs2)c(C(C)C)n1 ; CC(C)c1nc(C(F)(F)F)nc(C(C)C)c1CC(=O)N=S(N)(=O)c1cnc(C(C)(C)O)s1 ; CC(C)c1cc(C#N)nc(C(C)C)c1CC(=O)N=S(N)(=O)c1cc(C(C)(C)O)cs1] 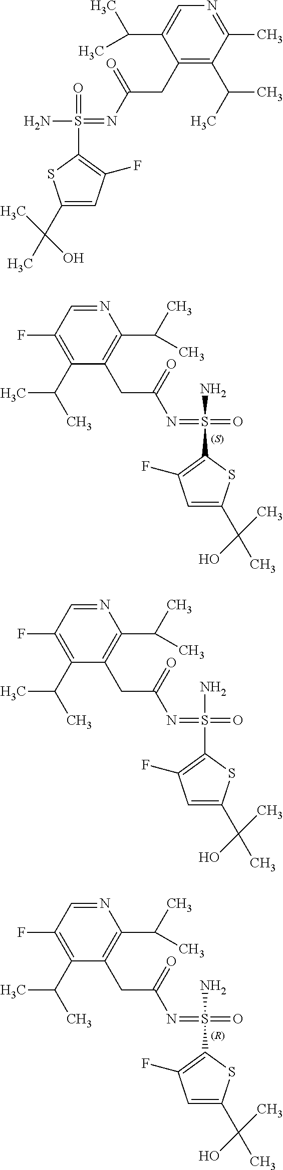 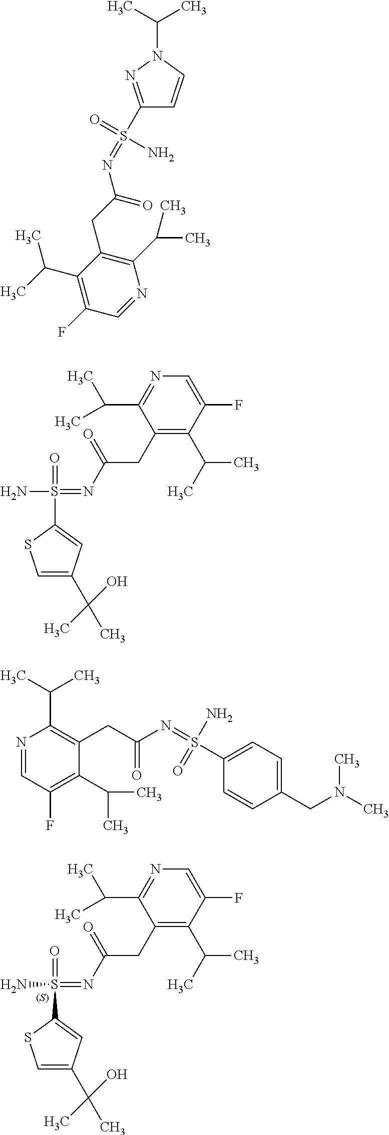 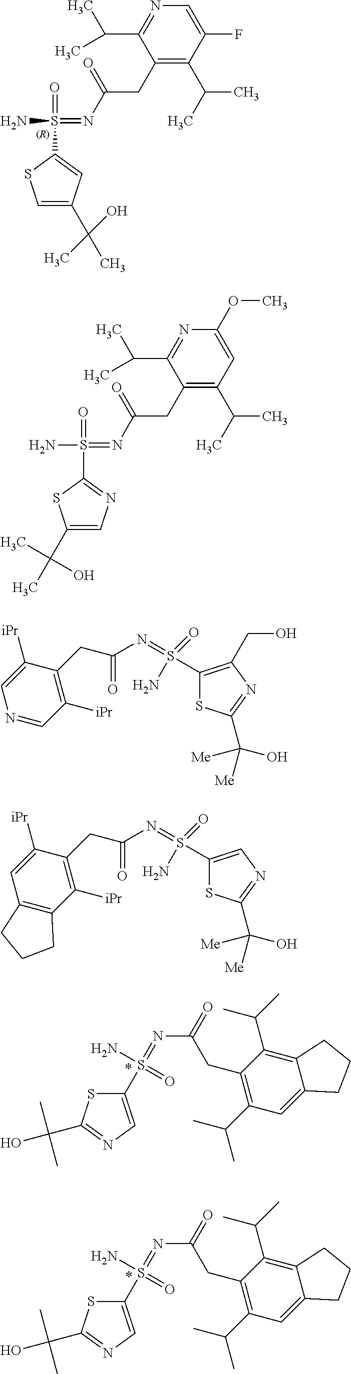 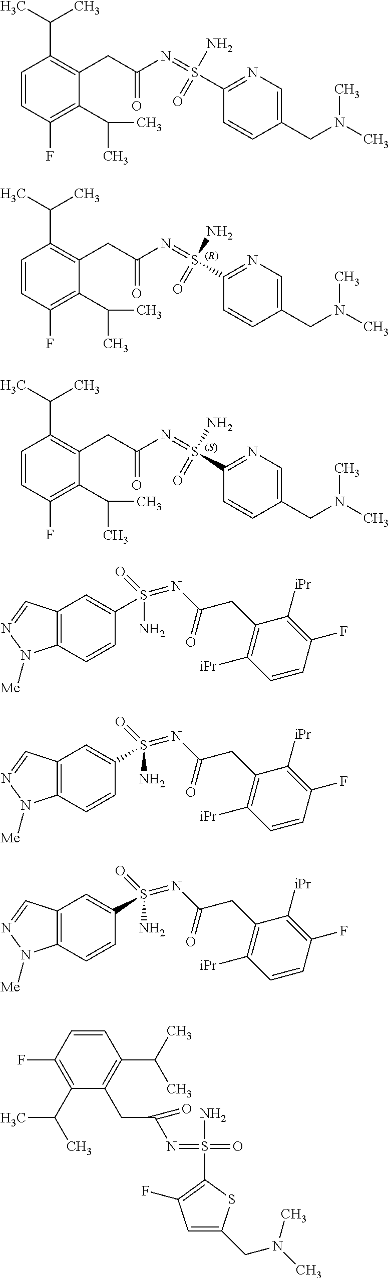 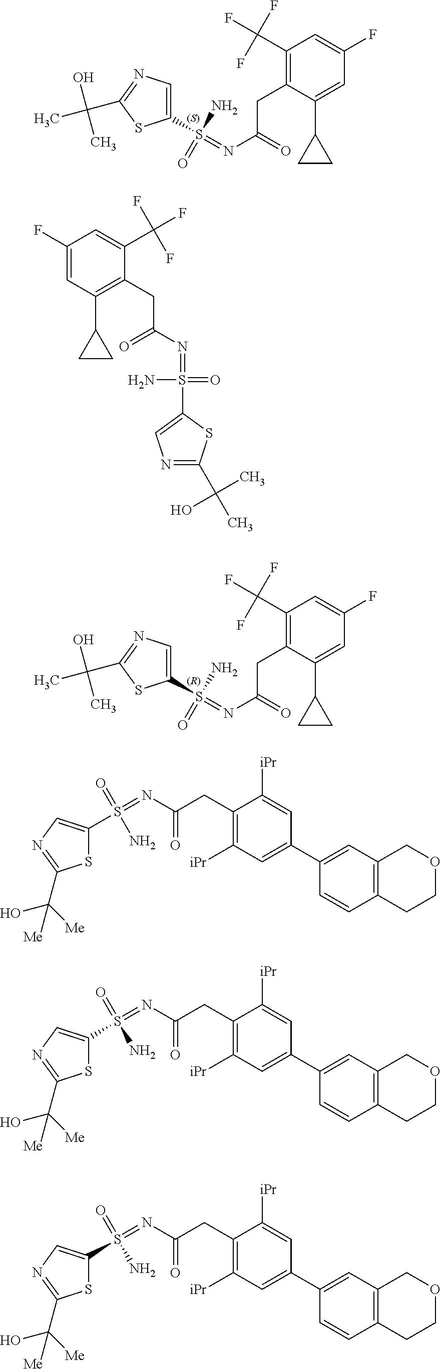 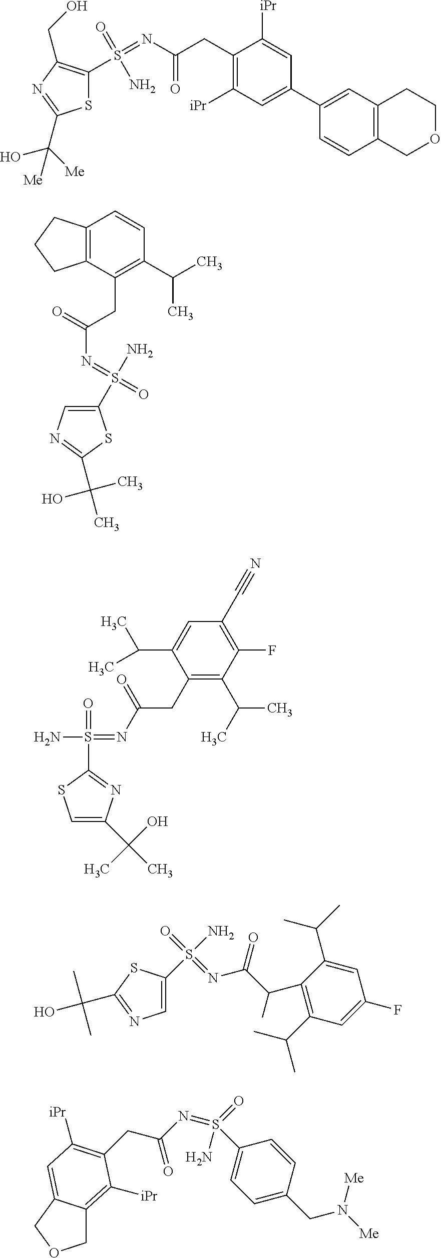 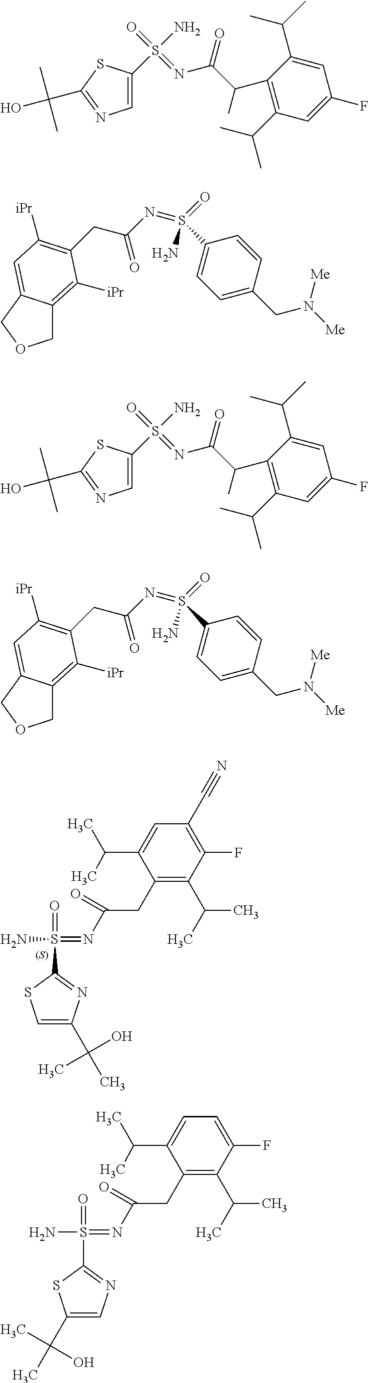 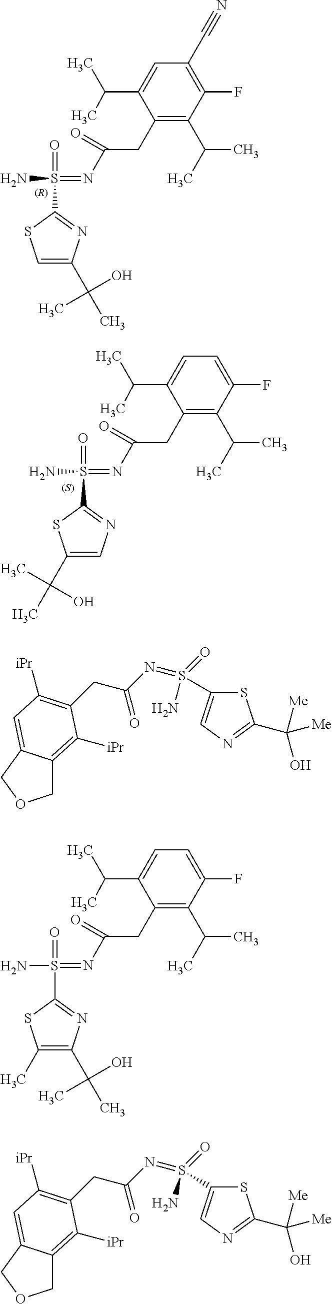 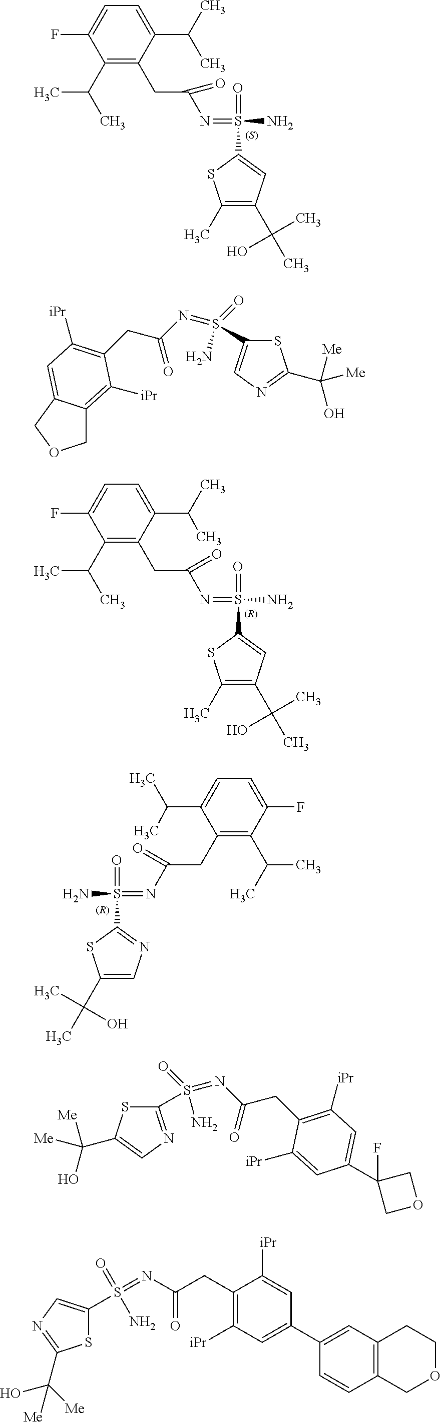 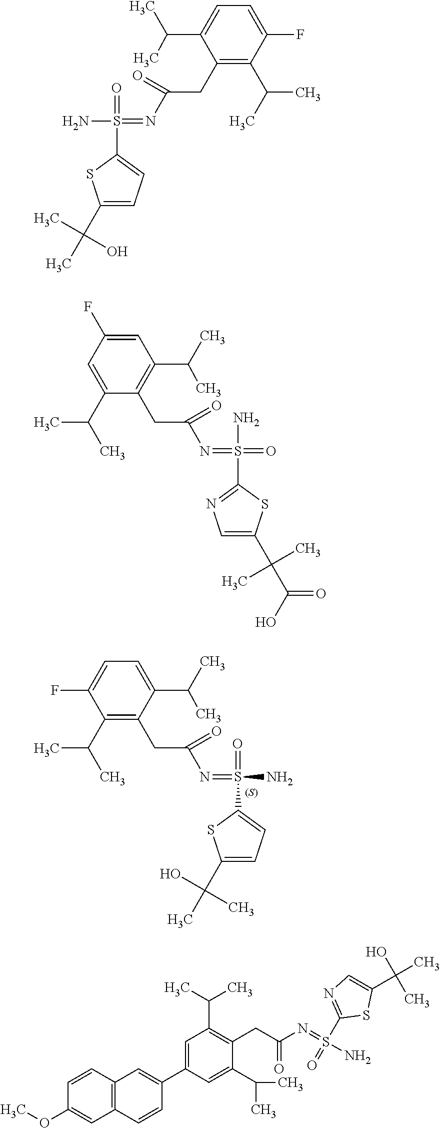 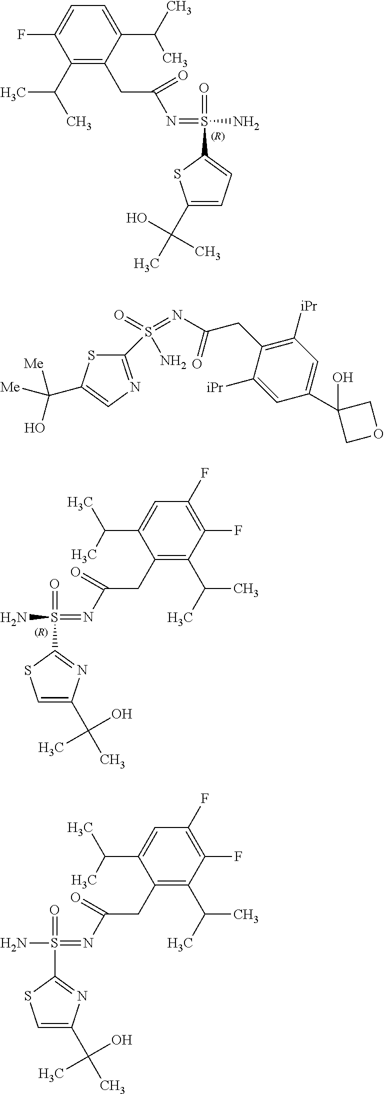 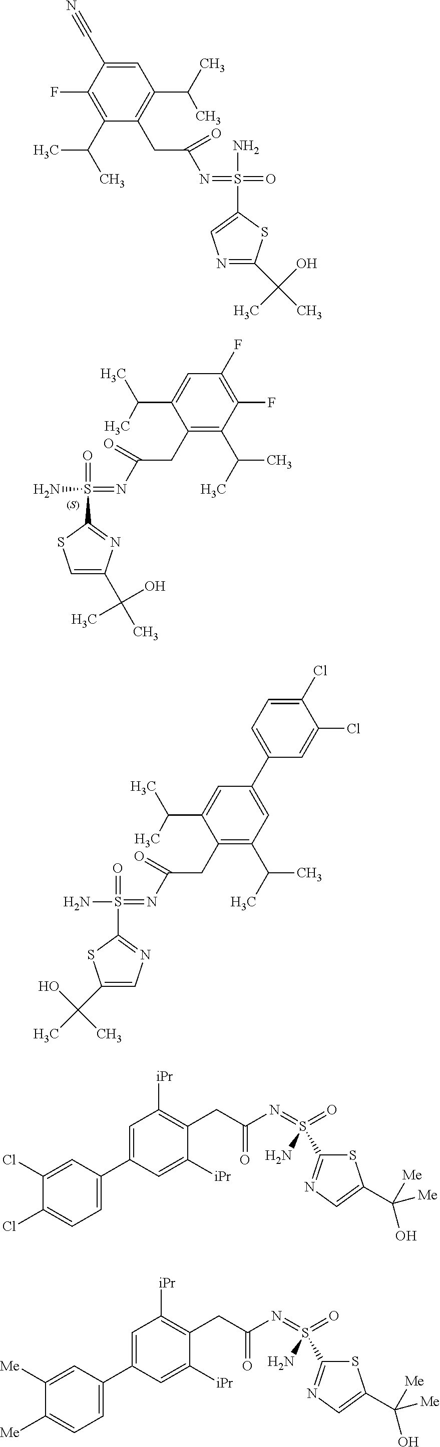 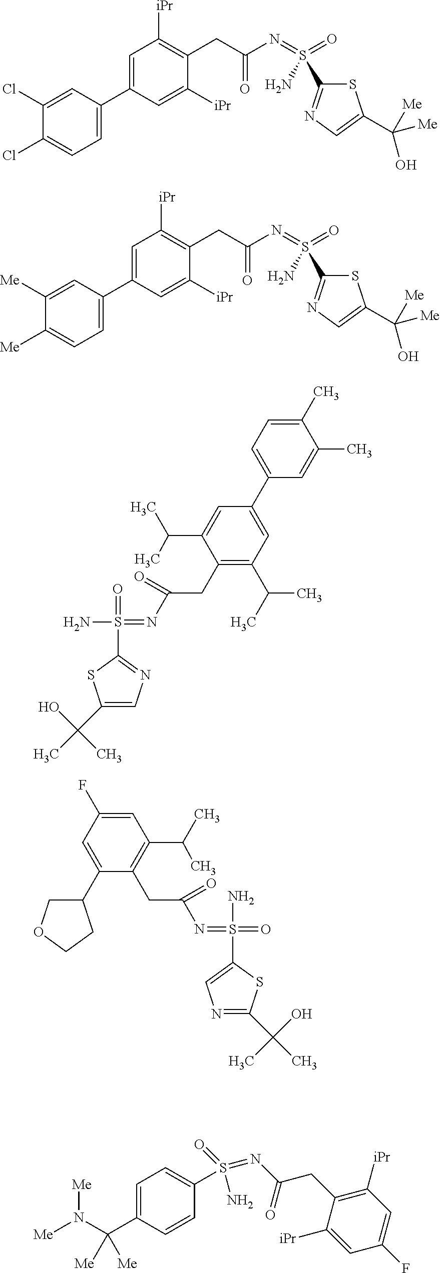 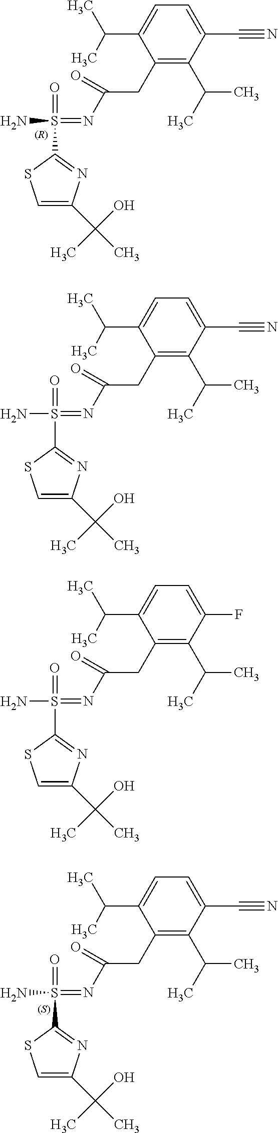 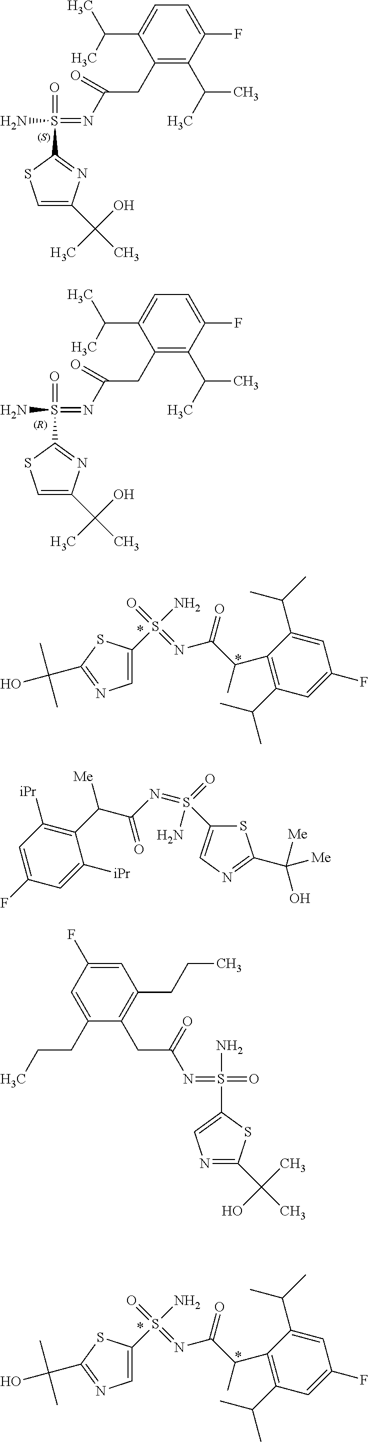 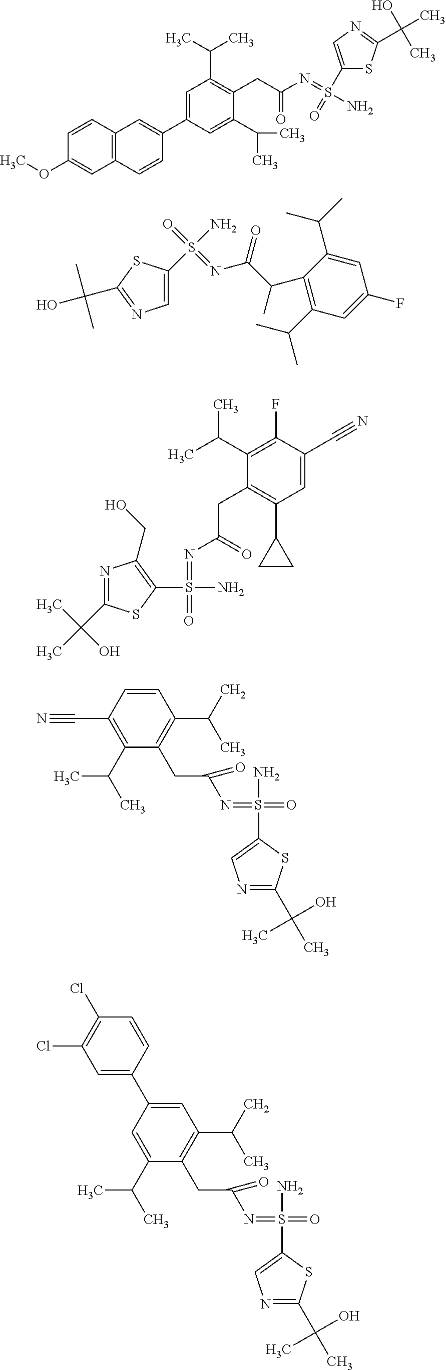 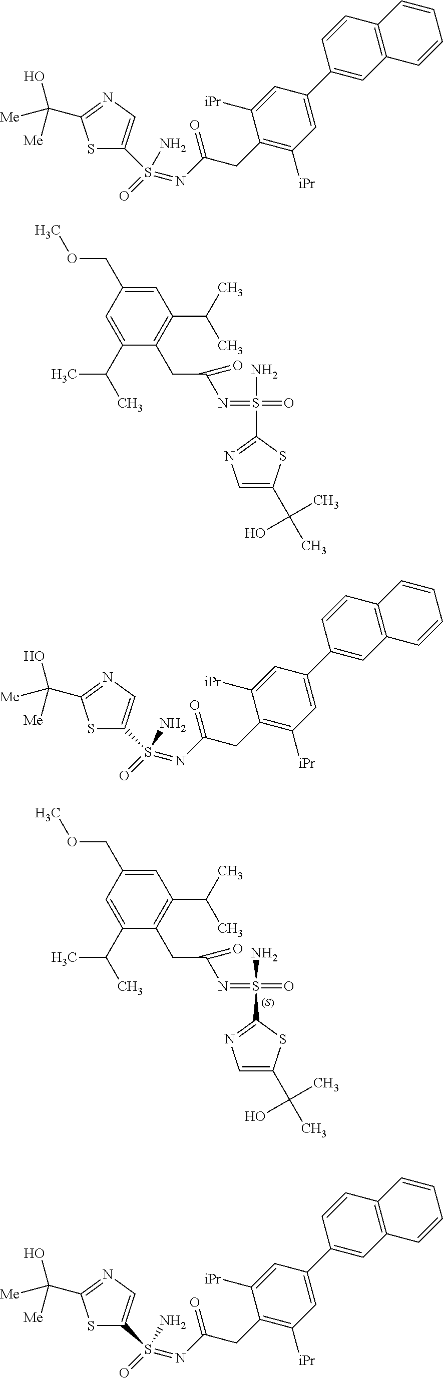 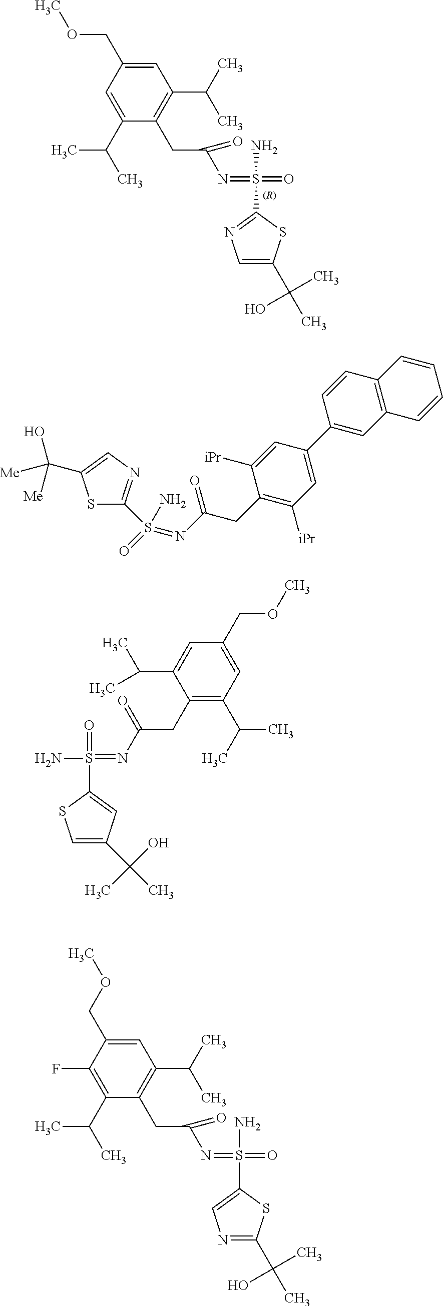 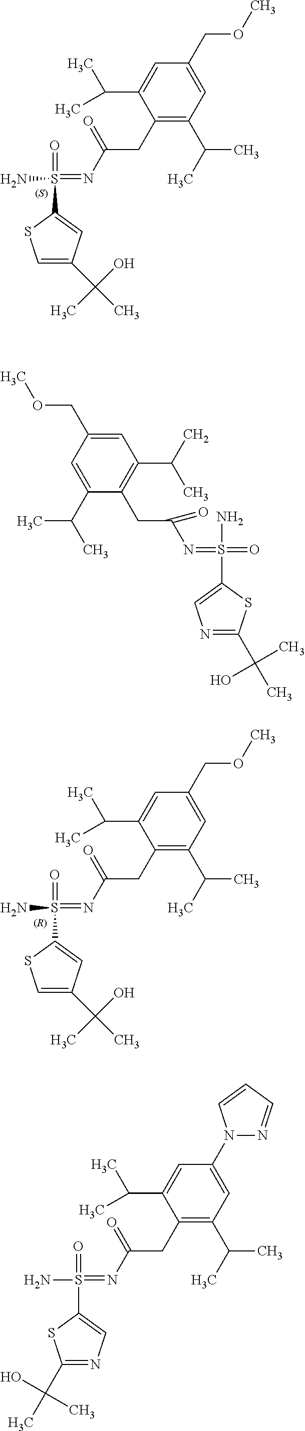 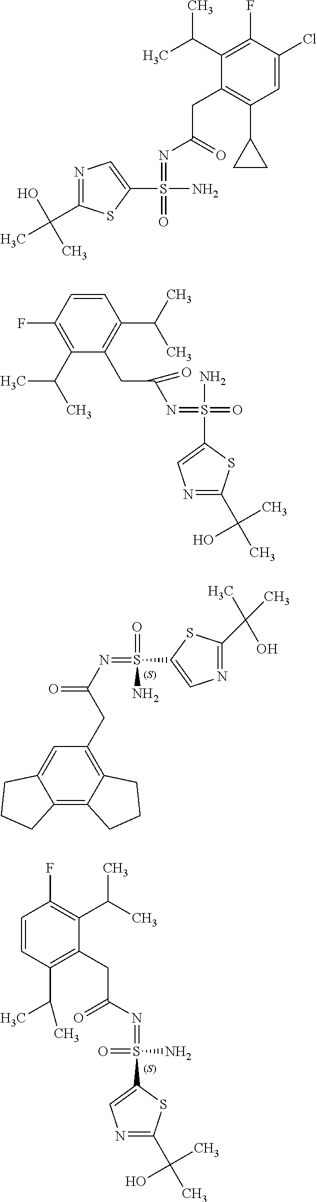 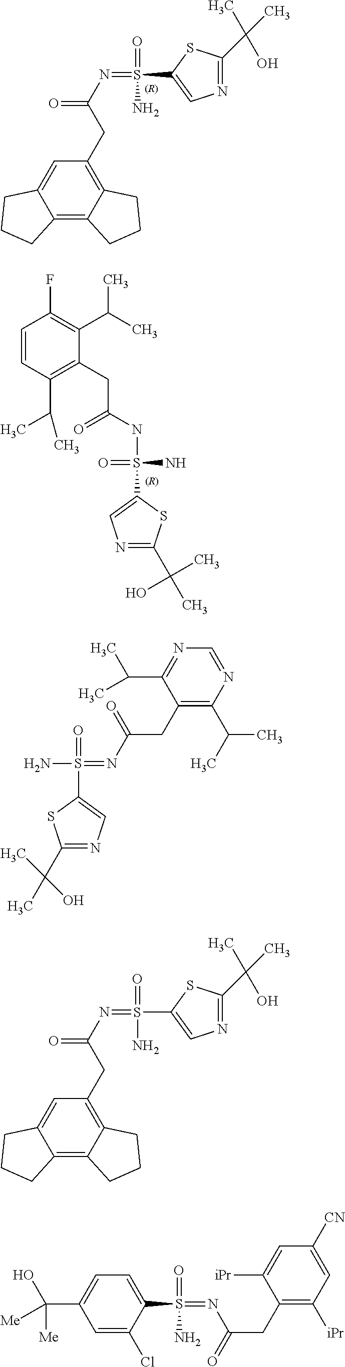 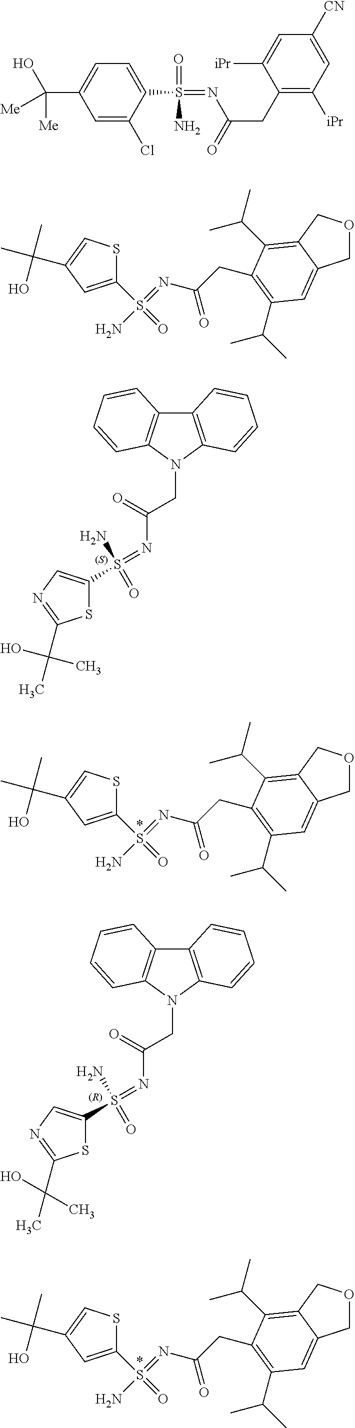 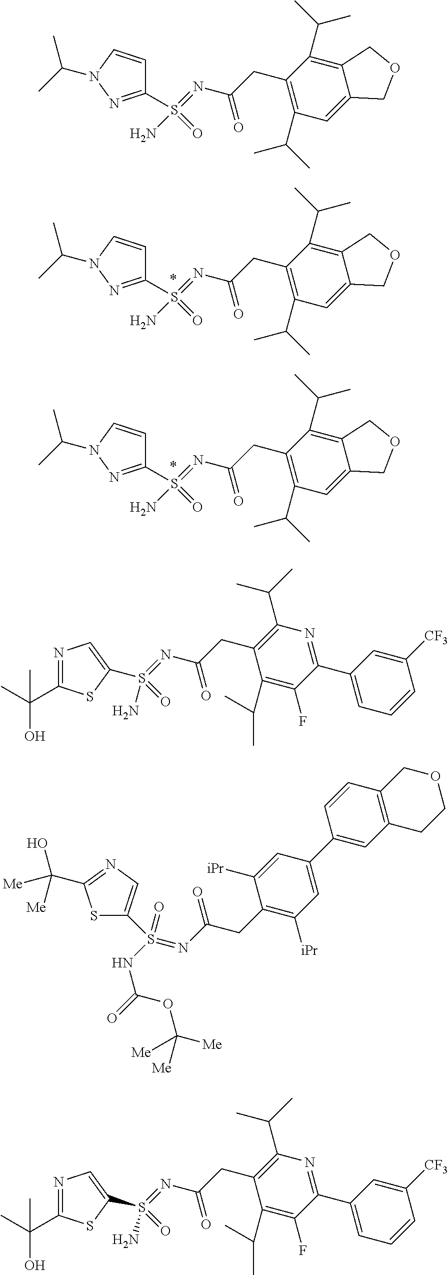 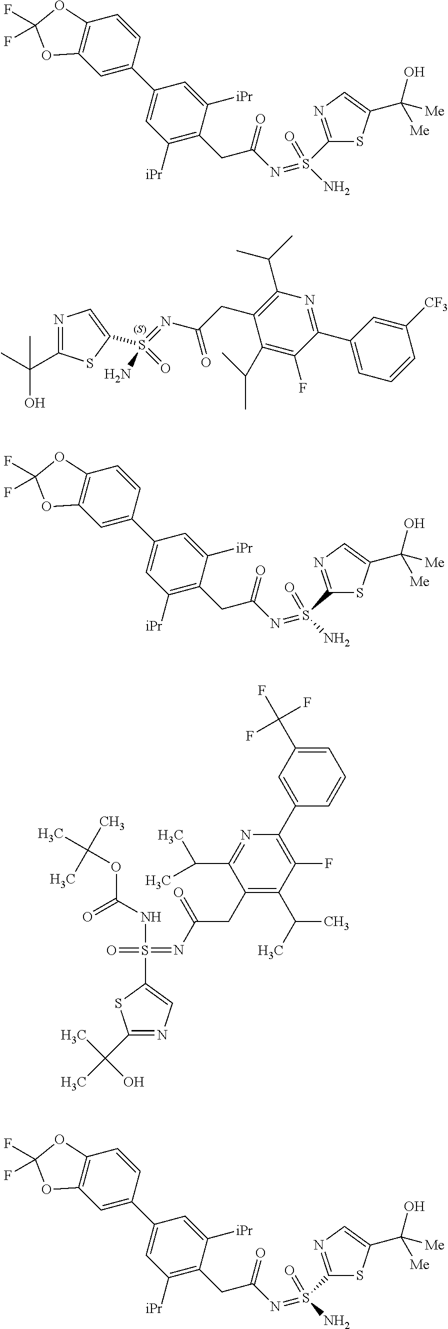 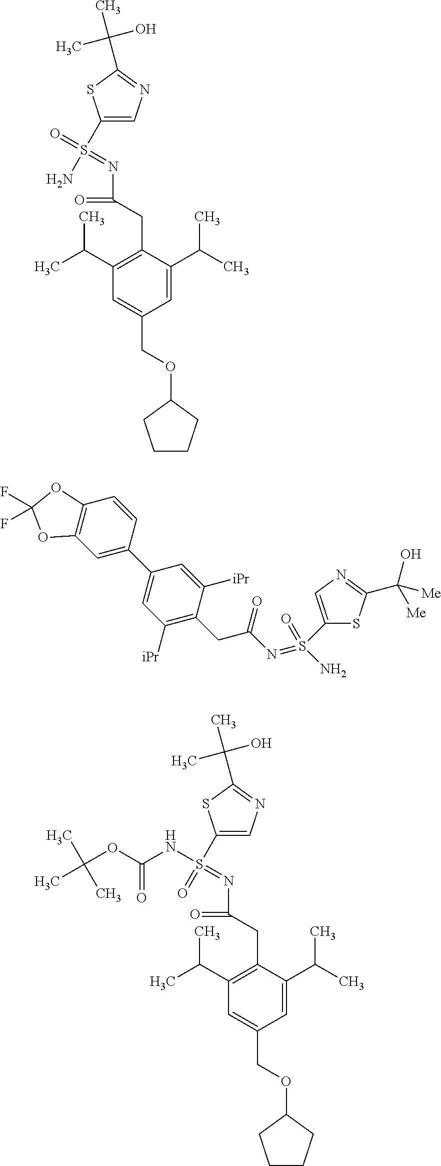 [SMILES: CC(C)c1cc(COC2CCCC2)cc(C(C)C)c1CC(=O)N=S(N)(=O)c1cnc(C(C)(C)O)s1 ; CC(C)c1cc(-c2ccc3c(c2)OC(F)(F)O3)cc(C(C)C)c1CC(=O)N=S(N)(=O)c1cnc(C(C)(C)O)s1 ; CC(C)c1cc(COC2CCCC2)cc(C(C)C)c1CC(=O)N=S(=O)(NC(=O)OC(C)(C)C)c1cnc(C(C)(C)O)s1]
or a pharmaceutically acceptable salt thereof.
